(19) United States
(12) Patent Application Publication
(10) Pub. No.: US 2010/0137313 A1
(43) Pub. Date: Jun. 3, 2010

(54) HETEROCYCLIC DERIVATIVES AND METHODS OF USE THEREOF

(75) Inventors: Ann Boriack-Sjodin, Waltham, MA (US); Daniel Robert Carcanague, Waltham, MA (US); Daemian David Dussault, Waltham, MA (US); Holia Hatoum-Mokdad, Waltham, MA (US); Kenneth Gregory Hull, Waltham, MA (US); Georgine Ioannidis, Waltham, MA (US); John Irvin Manchester, Waltham, MA (US); Helen Maureen McGuire, Waltham, MA (US); David Charles McKinney, Waltham, MA (US); Suzanne Stokes, Waltham, MA (US)

Correspondence Address: ASTRAZENECA R&D BOSTON, 35 GATEHOUSE DRIVE, WALTHAM, MA, 02451-1215, US

(73) Assignee: ASTRAZENECA AB

(21) Appl. No.: 12/572,436

(22) Filed: Oct. 2, 2009

Related U.S. Application Data
(60) Provisional application No. 61/102,583, filed on Oct. 3, 2008.

(51) Int. Cl. A61K 31/5377; C07D 401/14; A61K 31/506; C07D 413/14

(52) U.S. Cl. 514/236.5; 544/331; 514/275; 544/122

(57) ABSTRACT

Compounds of formula (I) and their pharmaceutically acceptable salts are described. Processes for their preparation, pharmaceutical compositions containing them, their use as medicaments and their use in the treatment of bacterial infections are also described.

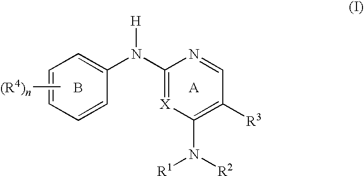

### CROSS-REFERENCE TO RELATED APPLICATION

This application claims the benefit of U.S. Provisional Patent Application No. 61/102583 filed on Oct. 3, 2008, the entire disclosure of which is herein incorporated by reference.

### FIELD OF THE INVENTION

The present invention relates to pyrimidine and pyridine derivatives which demonstrate antibacterial activity, processes for their preparation, pharmaceutical compositions containing them as the active ingredient, to their use as medicaments and to their use in the manufacture of medicaments for use in the treatment of bacterial infections in warm-blooded animals such as humans. In particular, this invention relates to compounds useful for the treatment of bacterial infections in warm-blooded animals such as humans, more particularly to the use of these compounds in the manufacture of medicaments for use in the treatment of bacterial infections in warm-blooded animals such as humans.

### BACKGROUND OF THE INVENTION

The international microbiological community continues to express serious concern that the evolution of antibiotic resistance could result in strains against which currently available antibacterial agents will be ineffective. In general, bacterial pathogens may be classified as either Gram-positive or Gram-negative pathogens. Antibiotic compounds with effective activity against both Gram-positive and Gram-negative pathogens are generally regarded as having a broad spectrum of activity. The compounds of the present invention are regarded as effective against both Gram-positive and certain Gram-negative pathogens.

Gram-positive pathogens, for example Staphylococci, Enterococci, Streptococci and mycobacteria, are particularly important because of the development of resistant strains which are both difficult to treat and difficult to eradicate from the hospital environment once established. Examples of such strains are methicillin resistant staphylococcus aureus (MRSA), methicillin resistant coagulase negative staphylococci (MRCNS), penicillin resistant Streptococcus pneumoniae and multiple resistant Enterococcus faecium.

The preferred clinically effective antibiotic for treatment of last resort of such resistant Gram-positive pathogens is vancomycin. Vancomycin is a glycopeptide and is associated with various toxicities, including nephrotoxicity. Furthermore, and most importantly, antibacterial resistance to vancomycin and other glycopeptides is also appearing. This resistance is increasing at a steady rate rendering these agents less and less effective in the treatment of Gram-positive pathogens. There is also now increasing resistance appearing towards agents such as β-lactams, quinolones and macrolides used for the treatment of upper respiratory tract infections, also caused by certain Gram negative strains including H.influenzae and M.catarrhalis.

Consequently, in order to overcome the threat of widespread multi-drug resistant organisms, there is an on-going need to develop new antibiotics, particularly those with either a novel mechanism of action and/or containing new pharmacophoric groups.

Deoxyribonucleic acid (DNA) gyrase is a member of the type II family of topoisomerases that control the topological state of DNA in cells (Champoux, J. J.; 2001. Ann Rev. Biochem. 70: 369-413). Type II topoisomerases use the free energy from adenosine triphosphate (ATP) hydrolysis to alter the topology of DNA by introducing transient double-stranded breaks in the DNA, catalyzing strand passage through the break and resealing the DNA. DNA gyrase is an essential and conserved enzyme in bacteria and is unique among topoisomerases in its ability to introduce negative supercoils into DNA. The enzyme consists of two subunits, encoded by gyrA and gyrB, forming an A₂B₂ tetrameric complex. The A subunit of gyrase (GyrA) is involved in DNA breakage and resealing and contains a conserved tyrosine residue that forms the transient covalent link to DNA during strand passage. The B subunit (GyrB) catalyzes the hydrolysis of ATP and interacts with the A subunit to translate the free energy from hydrolysis to the conformational change in the enzyme that enables strand-passage and DNA resealing.

Another conserved and essential type II topoisomerase in bacteria, called topoisomerase IV, is primarily responsible for separating the linked closed circular bacterial chromosomes produced in replication. This enzyme is closely related to DNA gyrase and has a similar tetrameric structure formed from subunits homologous to Gyr A and to Gyr B. The overall sequence identity between gyrase and topoisomerase IV in different bacterial species is high. Therefore, compounds that target bacterial type II topoisomerases have the potential to inhibit two targets in cells, DNA gyrase and topoisomerase IV; as is the case for existing quinolone antibacterials (Maxwell, A. 1997, Trends Microbiol. 5: 102-109).

DNA gyrase is a well-validated target of antibacterials, including the quinolones and the coumarins. The quinolones (e.g. ciprofloxacin) are broad-spectrum antibacterials that inhibit the DNA breakage and reunion activity of the enzyme and trap the GyrA subunit covalently complexed with DNA (Drlica, K., and X. Zhao, 1997, Microbiol. Molec. Biol. Rev. 61: 377-392). Members of this class of antibacterials also inhibit topoisomerase IV and as a result, the primary target of these compounds varies among species. Although the quinolones are successful antibacterials, resistance generated primarily by mutations in the target (DNA gyrase and topoisomerase IV) is becoming an increasing problem in several organisms, including S. aureus and Streptococcus pneumoniae (Hooper, D. C., 2002, The Lancet Infectious Diseases 2: 530-538). In addition, quinolones, as a chemical class, suffer from toxic side effects, including arthropathy that prevents their use in children (Lipsky, B. A. and Baker, C. A., 1999, Clin. Infect. Dis. 28: 352-364). Furthermore, the potential for cardiotoxicity, as predicted by prolongation of the QT_{c} interval, has been cited as a toxicity concern for quinolones.

There are several known natural product inhibitors of DNA gyrase that compete with ATP for binding the GyrB subunit (Maxwell, A. and Lawson, D. M. 2003, Curr. Topics in Med. Chem. 3: 283-303). The coumarins are natural products isolated from Streptomyces spp., examples of which are novobiocin, chlorobiocin and coumermycin A1. Although these compounds are potent inhibitors of DNA gyrase, their therapeutic utility is limited due to toxicity in eukaryotes and poor penetration in Gram-negative bacteria (Maxwell, A. 1997, Trends Microbiol. 5: 102-109). Another natural product class of compounds that targets the GyrB subunit is the cyclothialidines, which are isolated from Streptomyces filipensis (Watanabe, J. et al 1994, J. Antibiot. 47: 32-36). Despite potent activity against DNA gyrase, cyclothialidine is a poor antibacterial agent showing activity only against some eubacterial species (Nakada, N, 1993, Antimicrob. Agents Chemother. 37: 2656-2661).

Synthetic inhibitors that target the B subunit of DNA gyrase and topoisomeraseIV are known in the art. For example, coumarin-containing compounds are described in patent application number WO 99/35155, 5,6-bicyclic heteroaromatic compounds are described in patent application WO 02/060879, and pyrazole compounds are described in patent application WO 01/52845 (US patent U.S. Pat. No. 6,608,087). AstraZeneca has also published certain applications describing anti-bacterial compounds: WO2005/026149, WO2006/087544, WO2006/087548, WO2006/087543, WO2006/092599, WO2006/092608, and WO2007/071965.

### SUMMARY OF THE INVENTION

We have discovered a new class of compounds which are useful for inhibiting DNA gyrase and/or topoisomerase IV.

In one embodiment, according to the present invention there is provided a compound of formula (I):

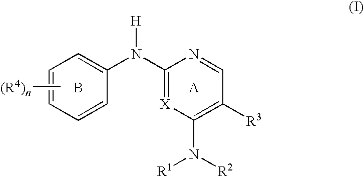

or a pharmaceutically acceptable salt thereof, wherein:

X is CH or N;

R¹ is hydrogen, a C₁₋₆alkyl, C₂₋₆alkenyl, C₂₋₆alkynyl, C₃₋₁₄carbocyclyl, or a heterocyclyl, wherein R¹ may be optionally substituted on carbon by one or more R⁶; and wherein if said hetercyclyl contains an ═N— or a —S— moiety that nitrogen may be optionally substituted by one oxo group and that sulfur may be optionally substituted by one or two oxo groups; and wherein if said heterocyclyl contains an —NH— moiety that nitrogen may be optionally substituted by a group selected from R⁷; provided that R¹ is not a substituted or unsubstituted phenyl;

R² is hydrogen or a C₁₋₆alkyl; or

R¹ and R², together with the nitrogen to which they are attached, form a heterocyclyl, wherein said heterocyclyl may be optionally substituted on carbon by one or more R⁸; wherein if said hetercyclyl contains an ═N— or a —S— moiety that nitrogen may be optionally substituted by one oxo group and that sulfur may be optionally substituted by one or two oxo groups; and wherein if said heterocyclyl contains an —NH— moiety that nitrogen may be optionally substituted by a group selected from R⁹;

R³ is a C₆₋₁₄aryl or a heteroaryl; wherein R³ may be optionally substituted on carbon by one or more R¹⁴; and wherein if said heteraryl contains an ═N— or a —S— moiety that nitrogen may be optionally substituted by one oxo group and that sulfur may be optionally substituted by one or two oxo groups; and wherein if said heteroaryl contains an —NH— moiety that nitrogen may be optionally substituted by a group selected from R¹⁵; provided that R³ is not an unsubstituted phenyl or an unsubstituted thiophenyl;

R⁴, for each occurrence, is independently selected from the group consisting of halo, cyano, nitro, hydroxy, C₁₋₆alkyl, C₁₋₆alkoxy, C₁₋₆alkanoyl, carbamoyl, N—C₁₋₆alkylcarbamoyl, N—C₁₋₆alkoxycarbamoyl, N,N-(C₁₋₆alkyl)₂carbamoyl, N—(SO₂R′)carbamoyl, N—C₁₋₆alkyl, C₁₋₆alkyl-S(O)ₐ—, R¹⁷R¹⁸N—S(O)₁—, C₃₋₁₄carbocyclyl, and heterocyclyl; or two R⁴ taken together with the carbon atoms to which they are attached form a C₃₋₁₄carbocyclyl or a heterocyclyl, wherein each R⁴ may be optionally substituted on carbon by one or more R¹⁶, wherein if either of said heterocyclyl contains an —NH— moiety that nitrogen may be optionally substituted by a group selected from R²⁶; provided that ring B together with —(R⁴)ₙ is not 3,4,5-trimethoxyphenyl;

n is an integer from 1 to 5;

a is 0, 1, or 2;

R⁶, R⁸, and R¹⁴, for each occurrence, are each independently selected from the group consisting of hydroxy, halo, cyano, nitro, C₁₋₆alkyl, C₂₋₆alkenyl, C₂₋₆alkynyl, mercapto, C₁₋₆alkoxy, C₁₋₆alkylS(O)ₐ wherein a is 0 to 2, —C(═N—OH)NH₂, —C(O)NHNH₂, phenoxy, carboxy, oxo, amino, N—C₁₋₆alkylamino, N,N-(C₁₋₆alkyl)₂amino, C₁₋₆alkoxycarbonyl, C₁₋₆alkanoyl, C₁₋₆alkanoyloxy, C₁₋₆alkanoylamino, C₁₋₆alkoxycarbonylamino, carbamoyl, N—C₁₋₆alkylcarbamoyl, N—C₁₋₆alkoxycarbamoyl, N,N-(C₁₋₆alkyl)₂carbamoyl, N—C₁₋₆alkyl-N-alkoxycarbamoyl, N—(SO₂R′)carbamoyl, N—C₁₋₆alkyl-N—(SO₂R′)carbamoyl, C₁₋₆alkylsulphonylamino, sulphamoyl, N—(C₁₋₆alkyl)sulphamoyl, N,N—(C₁₋₆alkyl)₂sulphamoyl, sulphamoylamino, N—(C₁₋₆alkyl)sulphamoylamino, N,N—(C₁₋₆alkyl)₂sulphamoylamino, C₃₋₁₄carbocyclyl-L- and heterocyclyl-L-; or two R¹⁴ taken together with the carbon atoms to which they are attached form a C₃₋₁₄carbocyclyl or a heterocyclyl; wherein R⁶, R⁸, and R¹⁴ may be each independently optionally substituted on carbon by one or more R¹⁰; and wherein if said hetercyclyl contains an ═N— or a —S— moiety that nitrogen may be optionally substituted by one oxo group and that sulfur may be optionally substituted by one or two oxo groups; and wherein if either of said heterocyclyl contains an —NH— moiety that nitrogen may be optionally substituted by a group selected from R¹¹;

R′ and R″, for each occurrence, are independently selected from the group consisting of C₁₋₆alkyl, C₆₋₁₄aryl and heterocyclyl, wherein R′ and R″ may be optionally substituted on carbon by one or more R²² and wherein if said heterocyclyl contains an —NH— moiety that nitrogen may be optionally substituted by a group selected from R²³;

R⁷, R⁹, R¹⁵ and R²³, for each occurrence, are each independently selected from the group consisting of C₁₋₆alkyl, C₁₋₆alkoxycarbonyl, C₁₋₆alkanoyl, carbamoyl, N—C₁₋₆alkylcarbamoyl, N,N—(C₁₋₆alkyl)₂carbamoyl, C₃₋₁₄carbocyclyl-C(O)—, heterocyclyl-C(O)—, (C₁₋₆alkyl)₃silyl, C₁₋₆alkylS(O)ₐ wherein a is 0 to 2, wherein R⁷, R⁹, and R¹⁵ may be each independently optionally substituted on carbon by one or more R¹²; and wherein if said hetercyclyl contains an ═N— or a —S— moiety that nitrogen may be optionally substituted by one oxo group and that sulfur may be optionally substituted by one or two oxo groups; and wherein if said heterocyclyl contains an —NH— moiety that nitrogen may be optionally substituted by a group selected from R¹³;

L, for each occurrence, is independent selected from a direct bond, —O—, —N(R²⁵)—, —C(O)—, —N(R²⁵)C(O)—, —C(O)N(R²⁵)—, —S(O)ₛ—, —SO₂N(R²⁵)— or —N(R²⁵)SO₂—; wherein R²⁵, for each occurrence, is independently selected from hydrogen or C₁₋₆alkyl and s is 0, 1 or 2;

R¹⁰ and R¹², for each occurrence, are independently selected from the group consisting of C₁₋₆alkyl, phenyl, halo, cyano, nitro, oxo, carboxy, hydroxy, C₁₋₆alkoxy, C₁₋₆alkoxycarbonyl, amino, N—C₁₋₆alkylamino, N,N—(C₁₋₆alkyl)₂amino, C₁₋₆alkanoylamino, C₁₋6alkylSO₂NH—, carbamoyl, N—C₁₋₆alkylcarbamoyl, N,N—(C₁₋₆alkyl)₂carbamoyl, N—C₁₋₆alkyloxycarbamoyl, C₁₋₆alkylS(O)ₐ wherein a is 0 to 2, and heterocyclyl, wherein said R¹⁰ and R¹² are independently optionally substituted on carbon by one or more C₁₋₆alkyl and wherein if said heterocyclyl contains an —NH— moiety that nitrogen may be optionally substituted by a group selected from R^{13′};

R¹¹, R¹³, R^{13′}, and R²⁶, for each occurrence, are each independently selected from the group consisting of C₁₋₆alkyl, C₁₋₆alkoxycarbonyl, C₁₋₆alkanoyl, C₃₋₆cycloalkanoyl, carbamoyl, C₁₋₆alkanoyloxy, C₁₋₆alkylS(O)ₐ, aryl S(O)ₐ wherein a is 0 to 2, carboxy, sulphamoyl and urea wherein said R¹¹, R¹³, R^{13′}, and R²⁶ are independently optionally substituted on carbon by one or more amino, C₁₋₆alkyl, C₁₋₆alkoxy or heterocyclyl;

R¹⁶, for each occurrence, is independently, a halo, hydroxy, a C₁₋₆alkyl, or a C₁₋₆alkoxy;

R¹⁷ and R¹⁸, for each occurrence, are independently hydrogen or a C₁₋₆alkyl; or R¹⁷ and R¹⁸, together with the nitrogen to which they are attached form a heterocyclyl;

R²², for each occurrence, is independently selected from the group consisting of halo, C₁₋₆alkyl, S(O)ₐR″ wherein a is 0 to 2, C₁₋₆alkanoyl, C₁₋₆alkanoylamino and heterocyclyl wherein R²² may be optionally substituted on carbon by one or more R²⁴;

R²⁴ is selected from halo, C₁₋₆alkanoylamino, and heterocyclyl;

provided that —NR¹R² is not —NHCH₃ or —N(CH₃)₂.

In another embodiment, according to the present invention there is provided a compound of formula (I):

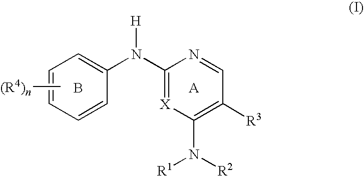

or a pharmaceutically acceptable salt thereof, wherein:

X is CH or N;

R¹ is hydrogen, a C₁₋₆alkyl, C₂₋₆alkenyl, C₂₋₆alkynyl, C₃₋₁₄carbocyclyl, or a heterocyclyl, wherein R¹ may be optionally substituted on carbon by one or more R⁶; and wherein if said hetercyclyl contains an ═N— or a —S— moiety that nitrogen may be optionally substituted by one oxo group and that sulfur may be optionally substituted by one or two oxo groups; and wherein if said heterocyclyl contains an —NH— moiety that nitrogen may be optionally substituted by a group selected from R⁷; provided that R¹ is not a substituted or unsubstituted phenyl;

R² is hydrogen or a C₁₋₆alkyl;

R¹ and R², together with the nitrogen to which they are attached, form a heterocyclyl, wherein said heterocyclyl may be optionally substituted on carbon by one or more R⁸; wherein if said hetercyclyl contains an ═N— or a —S— moiety that nitrogen may be optionally substituted by one oxo group and that sulfur may be optionally substituted by one or two oxo groups; and wherein if said heterocyclyl contains an —NH— moiety that nitrogen may be optionally substituted by a group selected from R⁹;

R³ is a C₆₋₁₄aryl or a heteroaryl; wherein R³ may be optionally substituted on carbon by one or more R¹⁴; and wherein if said heteraryl contains an ═N— or a —S— moiety that nitrogen may be optionally substituted by one oxo group and that sulfur may be optionally substituted by one or two oxo groups; and wherein if said heteroaryl contains an —NH— moiety that nitrogen may be optionally substituted by a group selected from R¹⁵; provided that R³ is not an unsubstituted phenyl or an unsubstituted thiophenyl;

R⁴, for each occurrence, is independently selected from the group consisting of halo, cyano, nitro, hydroxy, C₁₋₆alkyl, C₁₋₆alkoxy, C₁₋₆alkyl-S(O)ₐ, R¹⁷R¹⁸N—S(O)ₐ, C₃₋₁₄carbocyclyl, and heterocyclyl; or two R⁴ taken together with the carbon atoms to which they are attached form a C₃₋₁₄carbocyclyl or a heterocyclyl, wherein each R⁴ may be optionally substituted on carbon by one or more R¹⁶; provided that ring B together with —(R⁴)ₙ is not 3,4,5-trimethoxyphenyl;

n is an integer from 1 to 5;

a is 0, 1, or 2;

R⁶, R⁸, and R¹⁴, for each occurrence, are each independently selected from the group consisting of hydroxy, halo, cyano, nitro, C₁₋₆alkyl, C₂₋₆alkenyl, C₂₋₆alkynyl, mercapto, C₁₋₆alkoxy, C₁₋₆alkylS(O)ₐ wherein a is 0 to 2, —C(═N—OH)NH₂, phenoxy, carboxy, amino, N—C₁₋₆alkylamino, N,N—(C₁₋₆alkyl)₂amino, a heterocyclyl, C₁₋₆alkoxycarbonyl, C₁₋₆alkanoyl, C₁₋₆alkanoyloxy, C₁₋₆alkanoylamino, C₁₋₆alkoxycarbonylamino, carbamoyl, N—C₁₋₆alkylcarbamoyl, N,N—(C₁₋₆alkyl)₂carbamoyl, C₁₋₆alkylsulphonylamino, sulphamoyl, N—(C₁₋₆alkyl)sulphamoyl, N,N—(C₁₋₆alkyl)₂sulphamoyl, sulphamoylamino, N—(C₁₋₆alkyl)sulphamoylamino, N,N—(C₁₋₆alkyl)₂sulphamoylamino, C₃₋₁₄carbocyclyl-L- and heterocyclyl-L-; wherein R⁶, R⁸, and R¹⁴ may be each independently optionally substituted on carbon by one or more R¹⁰; and wherein if said hetercyclyl contains an ═N— or a —S— moiety that nitrogen may be optionally substituted by one oxo group and that sulfur may be optionally substituted by one or two oxo groups; and wherein if said heterocyclyl contains an —NH— moiety that nitrogen may be optionally substituted by a group selected from R¹¹;

R⁷, R⁹, and R¹⁵, for each occurrence, are each independently selected from the group consisting of C₁₋₆alkyl, C₁₋₆alkoxycarbonyl, C₁₋₆alkanoyl, carbamoyl, N—C₁₋₆alkylcarbamoyl, N,N—(C₁₋₆alkyl)₂carbamoyl, C₃₋₁₄carbocyclyl-C(O)—, heterocyclyl-C(O)—, (C₁₋₆alkyl)₃silyl, C₁₋₆alkylS(O)ₐ wherein a is 0 to 2, wherein R⁷, R⁹, and R¹⁵ may be each independently optionally substituted on carbon by one or more R¹²; and wherein if said hetercyclyl contains an ═N— or a —S— moiety that nitrogen may be optionally substituted by one oxo group and that sulfur may be optionally substituted by one or two oxo groups; and wherein if said heterocyclyl contains an —NH— moiety that nitrogen may be optionally substituted by a group selected from R¹³;

L, for each occurrence, is independent selected from a direct bond, —O—, —N(R²⁵)—, —C(O)—, —N(R²⁵)C(O)—, —C(O)N(R²⁵)—, —S(O)ₛ—, —SO₂N(R²⁵)— or —N(R²⁵)SO₂—; wherein R²⁵, for each occurrence, is independently selected from hydrogen or C₁₋₆alkyl and s is 0, 1 or 2;

R¹⁰ and R¹², for each occurrence, are independently selected from the group consisting of C₁₋₆alkyl, phenyl, halo, cyano, nitro, carboxy, hydroxy, C₁₋₆alkoxy, C₁₋₆alkoxycarbonyl, amino, N—C₁₋₆alkylamino, N,N—(C₁₋₆alkyl)₂amino, carbamoyl, N—C₁₋₆alkylcarbamoyl, N,N—(C₁₋₆alkyl)₂carbamoyl and N—C₁₋₆alkyloxycarbamoyl; and

R¹¹ and R¹³, for each occurrence, are each independently a C₁₋₆alkyl;

R¹⁶, for each occurrence, is independently, a halo, hydroxy, a C₁₋₆alkyl, or a C₁₋₆alkoxy;

R¹⁷ and R¹⁸, for each occurrence, are independently hydrogen or a C₁₋₆alkyl; or R¹⁷ and R¹⁸, together with the nitrogen to which they are attached form a heterocyclyl;

provided that —NR¹R² is not —NHCH₃, —N(CH₃)₂.

In another embodiment, the invention provides pharmaceutical compositions comprising a compound represented by formula (I), or a pharmaceutically acceptable salt thereof, and a pharmaceutically acceptable excipient or carrier.

In another embodiment, the invention provides a method of inhibiting bacterial DNA gyrase and/or bacterial topoisomerase IV in a warm-blooded animal in need of such treatment, comprising administering to the animal an effective amount of a compound represented by formula (I), or a pharmaceutically acceptable salt thereof. In a particular embodiment, the warm-blooded animal is a human.

In another embodiment, the invention provides a method of producing an antibacterial effect in a warm-blooded animal in need of such treatment, comprising administering to the animal an effective amount of a compound represented by formula (I), or a pharmaceutically acceptable salt thereof. In a particular embodiment, the warm-blooded animal is a human.

In another embodiment, the invention provides a method of treating a bacterial infection in a warm-blooded animal in need thereof, comprising administering to the animal an effective amount of a compound represented by formula (I), or a pharmaceutically acceptable salt thereof. In a particular embodiment, the warm-blooded animal is a human. In one embodiment, the bacterial infection is selected from the group consisting of community-acquired pneumoniae, hospital-acquired pneumoniae, skin and skin structure infections, acute exacerbation of chronic bronchitis, acute sinusitis, acute otitis media, catheter-related sepsis, febrile neutropenia, osteomyelitis, endocarditis, urinary tract infections and infections caused by drug resistant bacteria such as Penicillin-resistant Streptococcus pneumoniae, methicillin-resistant Staphylococcus aureus, methicillin-resistant Staphylococcus epidermidis and Vancomycin-Resistant Enterococci. In a particular embodiment, the warm-blooded animal is a human.

In another embodiment, the invention provides compounds of formula (I) or a pharmaceutically acceptable salt thereof, for use as a medicament.

In another embodiment, the invention provides the use of a compound represented by formula (I), or a pharmaceutically acceptable salt thereof, for the manufacture of a medicament for use in the production of an antibacterial effect in a warm-blooded animal. In a particular embodiment, the warm-blooded animal is a human.

In another embodiment, the invention provides the use of a compound represented by formula (I), or a pharmaceutically acceptable salt thereof, for the manufacture of a medicament for use in inhibition of bacterial DNA gyrase and/or topoisomerase IV in a warm-blooded animal. In a particular embodiment, the warm-blooded animal is a human.

In another embodiment, the invention provides the use of a compound represented by formula (I), or a pharmaceutically acceptable salt thereof, for the manufacture of a medicament for use the treatment of a bacterial infection in a warm-blooded animal. In one embodiment, the bacterial infection is selected from the group consisting of community-acquired pneumoniae, hospital-acquired pneumoniae, skin and skin structure infections, acute exacerbation of chronic bronchitis, acute sinusitis, acute otitis media, catheter-related sepsis, febrile neutropenia, osteomyelitis, endocarditis, urinary tract infections, Penicillin-resistant Streptococcus pneumoniae, methicillin-resistant Staphylococcus aureus, methicillin-resistant Staphylococcus epidermidis and Vancomycin-Resistant Enterococci. In a particular embodiment, the warm-blooded animal is a human.

In another embodiment, the invention provides a compound represented by formula (I), or a pharmaceutically acceptable salt thereof, for use in production of an anti-bacterial effect in a warm-blooded animal.

In another embodiment, the invention provides a compound represented by formula (I), or a pharmaceutically acceptable salt thereof, for use in inhibition of bacterial DNA gyrase and/or topoisomerase IV in a warm-blooded animal.

In another embodiment, the invention provides a compound represented by formula (I), or a pharmaceutically acceptable salt thereof, for use in the treatment of a bacterial infection in a warm-blooded animal.

In another embodiment, the invention provides a compound represented by formula (I), or a pharmaceutically acceptable salt thereof, for use in the treatment of community-acquired pneumoniae, hospital-acquired pneumoniae, skin and skin structure infections, acute exacerbation of chronic bronchitis, acute sinusitis, acute otitis media, catheter-related sepsis, febrile neutropenia, osteomyelitis, endocarditis, urinary tract infections, Penicillin-resistant Streptococcus pneumoniae, methicillin-resistant Staphylococcus aureus, methicillin-resistant Staphylococcus epidermidis or Vancomycin-Resistant Enterococci.

### DETAILED DESCRIPTION OF THE INVENTION

In this specification the term alkyl includes both straight chained and branched saturated hydrocarbon groups. For example, “C₁₋₆alkyl” refers to an alkyl that has from 1 to 6 carbon atom and includes, for example, methyl, ethyl, propyl, isopropyl and t-butyl. However references to individual alkyl groups such as propyl are specific for the straight chain version only unless otherwise indicated (e.g., isopropyl). An analogous convention applies to other generic terms. Unless otherwise specified, when two or more alkyl groups are indicated by, for example, the term (C₁₋₆alkyl)₂ (such as in the term N,N—(C₁₋₆alkyl)₂amino), the alkyl groups can be the same or different.

The term “C₂₋₆alkenyl,” as used herein refers to a straight chain or branched hydrocarbon having at least one double bond.

The term “C₂₋₆alkynyl,” as used herein refers to a straight chain or branched hydrocarbon having at least one triple bond.

As used herein, the term “halo” refers to fluoro, chloro, bromo, and iodo.

A “heterocyclyl” is a saturated, partially saturated or unsaturated, mono or bicyclic ring containing 4-14 atoms of which at least one atom is chosen from nitrogen, sulphur or oxygen, which may, unless otherwise specified, be carbon or nitrogen linked, wherein a —CH₂— group can optionally be replaced by a —C(O)— and a ring nitrogen may be optionally substituted with one oxo to form an N-oxide and a ring sulfur may be optionally substituted with one or two oxo groups to form S-oxide(s). In one embodiment of the invention a “heterocyclyl” is a saturated, partially saturated or unsaturated, monocyclic ring containing 5 or 6 atoms of which at least one atom is chosen from nitrogen, sulphur or oxygen, it may, unless otherwise specified, be carbon or nitrogen linked In a further aspect of the invention a “heterocyclyl” is an unsaturated, carbon-linked, monocyclic ring containing 5 or 6 atoms of which at least one atom is chosen from nitrogen, sulphur or oxygen. Examples and suitable values of the term “heterocyclyl” are azepanyl, azetidinyl, morpholinyl, piperidinyl, piperazinyl, pyridinyl, pyranyl, pyrrolyl, pyrazolyl, isothiazolyl, indolinyl, quinolinyl, thienyl, 1,3-benzodioxolyl, benzothiazolyl, thiadiazolyl, oxadiazolyl, piperazinyl, thiazolidinyl, pyrrolidinyl, thiomorpholino, pyrrolinyl, homopiperazinyl, 3,5-dioxapiperidinyl, tetrahydropyranyl, imidazolyl, 4,5-dihydro-oxazolyl, pyrimidinyl, pyrazinyl, pyridazinyl, isoxazolyl, thiazolyl, 1H-tetrazolyl, 1H-triazolyl, N-methylpyrrolyl, 4-pyridone, quinolin-4(1H)-one, pyridin-2(1H)-one, imidazo[1,2-a]pyridinyl, 1-isoquinolone, 2-pyrrolidone, 4-thiazolidone, quinoxalinyl, 5,6-dihydro[1,3]thiazolo[4,5-d]pyridazinyl, pyridine-N-oxide and quinoline-N-oxide. Suitable examples of “a nitrogen linked heterocyclyl” are morpholino, piperazin-1-yl, piperidin-1-yl and imidazol-1-yl. In a further aspect of the invention a “heterocyclyl” is a heteroaryl. The term “heteroaryl” refers to an unsaturated and aromatic heterocyclyl which has 5-14 ring atoms wherein at least one atom is chosen from nitrogen, sulphur or oxygen. Examples and suitable values for heteroaryl groups include pyridinyl, 1H-pyrrolyl, 1H-pyrazolyl, isothiazolyl, quinolinyl, thienyl, benzofuranyl, benzothiophenyl, benzothiazolyl, benzimidazolyl, thiadiazolyl, oxadiazolyl, 1H-imidazolyl, pyrimidinyl, pyrazinyl, pyridazinyl, isoxazolyl, thiazolyl, thiophenyl, 1H-pyrazolyl, 1H-tetrazolyl, 1H-triazolyl, N-methylpyrrolyl, 4-oxo-1,4-dihydroquinolinyl, pyridin-2(1H)-one, imidazo[1,2-a]pyridinyl, 1H-indazol-1-yl, 1-isoquinolone, quinoxalinyl, pyridine-N-oxide and quinoline-N-oxide. In a particular embodiment, the heteroaryl is a 5- or 6-membered heteroaryl, for example, pyridinyl, 1H-pyrrolyl, 1H-pyrazolyl, isothiazolyl, thienyl, thiadiazolyl, oxadiazolyl, 1H-imidazolyl, pyrimidinyl, pyrazinyl, pyridazinyl, isoxazolyl, thiazolyl, 1H-tetrazolyl, 1H-triazolyl, N-methylpyrrolyl, and pyridine-N-oxide. In another embodiment heteroaryl also includes pyridinyl-2(1H)-one and indolyl.

A “carbocyclyl” is a saturated, partially saturated or unsaturated, mono-, bi- or tricyclic carbon ring that contains 3-14 atoms; wherein a —CH₂— group can optionally be replaced by a —C(O)—. In one embodiment, “carbocyclyl” is a monocyclic ring containing 5 or 6 atoms or a bicyclic ring containing 9 or 10 atoms. Examples of carbocyclyls include cyclopropyl, cyclobutyl, 1-oxocyclopentyl, cyclopentyl, cyclopentenyl, cyclohexyl, cyclohexenyl, phenyl, naphthyl, tetralinyl, indanyl or 1-oxoindanyl. The term carbocyclyl encompasses both cycloalkyl and aryl groups. The term cycloalkyl refers to a C₃₋₁₄carbocyclyl which is completely saturated, for example cyclopropyl, cyclobutyl, cyclopentyl, and cyclohexyl. The term “aryl” refers to a carbocyclyl which is completely unsaturated and is aromatic. A C₆₋₁₄aryl is an aromatic, mono-, bi- or tricyclic carbon ring that contains 6-14 atoms, for example phenyl or naphthenyl.

An example of “C₁₋₆alkanoyloxy” is acetoxy. Examples of “C₁₋₆alkoxycarbonyl” are methoxycarbonyl, ethoxycarbonyl, n- and t-butoxycarbonyl. Examples of “C₁₋₆alkoxycarbonylamino” are methoxycarbonylamino, ethoxycarbonylamino, n- and t-butoxycarbonylamino. Examples of “C₁₋₆alkoxy” are methoxy, ethoxy, isopropoxy, and tert-butoxy. Examples of “C₁₋₆alkanoylamino” are formamido, acetamido and propionylamino. Examples of “C₁₋₆alkylS(O)ₐ wherein a is 0, 1, or 2” are methylthio, ethylthio, methylsulphinyl, ethylsulphinyl, methylsulfonyl and ethylsulphonyl. Examples of “C₁₋₆alkanoyl” are propionyl and acetyl. Examples of “N—(C₁₋₆alkyl)amino” are methylamino and ethylamino. Examples of “N,N—(C₁₋₆alkyl)₂amino” are N,N-dimethylamino, N,N-diethylamino and N-ethyl-N-methylamino. Examples of “C₂₋₆alkenyl” are vinyl, allyl and 1-propenyl. Examples of “C₂₋₆alkynyl” are ethynyl, 1-propynyl and 2-propynyl. Examples of “N—(C₁₋₆alkyl)sulphamoyl” are N-(methyl)sulphamoyl and N-(ethyl)sulphamoyl. Examples of “N,N—(C₁₋₆alkyl)₂sulphamoyl” are N,N-(dimethyl)sulphamoyl and N-(methyl)-N-(ethyl)sulphamoyl. An example of “N,N—(C₁₋₆alkyl)₂sulphamoylamino” are N,N-dimethylsulphamoylamino. Examples of “N—(C₁₋₆alkyl)carbamoyl” are methylaminocarbonyl and ethylaminocarbonyl. Examples of “N,N—(C₁₋₆alkyl)₂carbamoyl” are dimethylaminocarbonyl and methylethylaminocarbonyl. Examples of “N—(C₁₋₆alkoxy)carbamoyl” are methoxyaminocarbonyl and isopropoxyaminocarbonyl. Examples of “N—(C₁₋₆alkyl)-N—(C₁₋₆alkoxy)carbamoyl” are N-methyl-N-methoxyaminocarbonyl and N-methyl-N-ethoxyaminocarbonyl. Examples of “C₃₋₆cycloalkyl” are cyclopropyl, cyclobutyl, cyclopropyl and cyclohexyl. Examples of “C₁₋₆alkylsulphonylamino” are methylsulphonylamino, isopropylsulphonylamino and t-butylsulphonylamino. Examples of “C₁₋₆alkylsulphonylaminocarbonyl” are methylsulphonylaminocarbonyl, isopropylsulphonylaminocarbonyl and t-butylsulphonylaminocarbonyl. Examples of “C₁₋₆alkylsulphonyl” are methylsulphonyl, isopropylsulphonyl and t-butylsulphonyl.

Examples of “C₁₋₃alkylsulphonylcarbamoyl” are methylsulphonylcarbamoyl, i.e. CH₃SO₂NHC(O)—, and ethylsulphonylcarbamoyl, i.e. CH₃CH₂SO₂NHC(O)—.

When a carbon atom is substituted by “oxo” a —C(O)— is formed. Thus, for example, if a pyridyl group is substituted on carbon by oxo, a pyridinyl-one is formed, e.g. if the carbon in the two position of pyridine is substituted by oxo, pyridinyl-2(1H)-one is formed.

The term “formula (I)”, unless otherwise specified, refers to all embodiments of formula (I).

A compound of formula (I) may form stable acid or basic salts, and in such cases administration of a compound as a salt may be appropriate, and pharmaceutically acceptable salts may be made by conventional methods such as those described below.

Suitable pharmaceutically-acceptable salts include acid addition salts such as methanesulfonate, tosylate, α-glycerophosphate, fumarate, hydrochloride, citrate, maleate, tartrate and (less preferably) hydrobromide. Also suitable are salts formed with phosphoric and sulfuric acid. In another aspect suitable salts are base salts such as an alkali metal salt for example sodium, an alkaline earth metal salt for example calcium or magnesium, an organic amine salt for example triethylamine, morpholine, N-methylpiperidine, N-ethylpiperidine, procaine, dibenzylamine, N,N-dibenzylethylamine, tris-(2-hydroxyethyl)amine, N-methyl d-glucamine and amino acids such as lysine. There may be more than one cation or anion depending on the number of charged functions and the valency of the cations or anions.

However, to facilitate isolation of the salt during preparation, salts which are less soluble in the chosen solvent may be preferred whether pharmaceutically-acceptable or not.

Within the present invention it is to be understood that a compound of the formula (I), or a salt thereof may exhibit the phenomenon of tautomerism and that the formulae drawings within this specification can represent only one of the possible tautomeric forms. It is to be understood that the invention encompasses any tautomeric form which inhibits DNA gyrase and/or topoisomerase IV and is not to be limited merely to any one tautomeric form utilized within the formulae drawings. The formulae drawings within this specification can represent only one of the possible tautomeric forms and it is to be understood that the specification encompasses all possible tautomeric forms of the compounds drawn not just those forms which it has been possible to show graphically herein. The same applies to compound names.

It will be appreciated by those skilled in the art that certain compounds of formula (I) contain an asymmetrically substituted carbon and/or sulfur atom, and accordingly may exist in, and be isolated in, optically-active and racemic forms. Some compounds may exhibit polymorphism. It is to be understood that the present invention encompasses any racemic, optically-active, polymorphic or stereoisomeric form, or mixtures thereof, which form possesses properties useful in the inhibition of DNA gyrase and/or topoisomerase IV, it being well known in the art how to prepare optically-active forms (for example, by resolution of the racemic form by recrystallization techniques, by synthesis from optically-active starting materials, by chiral synthesis, by enzymatic resolution, by biotransformation, or by chromatographic separation using a chiral stationary phase) and how to determine efficacy for the inhibition of DNA gyrase and/or topoisomerase IV by the standard tests described hereinafter.

By way of clarity, compounds of the invention included all isotopes of the atoms present in formula (I) and any of the examples or embodiments disclosed herein. For example, H (or hydrogen) represents any isotopic form of hydrogen including ¹H, ²H (D), and ³H (T); C represents any isotopic form of carbon including ¹²C, ¹³C, and ¹⁴C; O represents any isotopic form of oxygen including ¹⁶O, ¹⁷O and ¹⁸O; N represents any isotopic form of nitrogen including ¹³N, ¹⁴N and ¹⁵N; P represents any isotopic form of phosphorous including ³¹P and ³²P; S represents any isotopic form of sulfur including ³²S and ³⁵S; F represents any isotopic form of fluorine including ¹⁹F and ¹⁸F; Cl represents any isotopic form of chlorine including ³⁵Cl, ³⁷Cl and ³⁶Cl; and the like. In a preferred embodiment, compounds represented by formula (I) comprises isomers of the atoms therein in about their naturally occurring abundance. However, in certain instances, it is desirable to enrich one or more atom in a particular isotope which would normally be present in less abundance. For example, ¹H would normally be present in greater than 99.98% abundance; however, a compound of the invention can be enriched in ²H or ³H at one or more positions where H is present. In particular embodiments of the compounds of formula (I), when, for example, hydrogen is enriched in the deuterium isotope, the symbol “D” is used to represent the enrichment in deuterium. In one embodiment, when a compound of the invention is enriched in a radioactive isotope, for example ³H and ¹⁴C, they may be useful in drug and/or substrate tissue distribution assays. It is to be understood that the invention encompasses all such isotopic forms which inhibit DNA gyrase and/or topoisomerase IV.

It is also to be understood that certain compounds of the formula (I), and salts thereof can exist in solvated as well as unsolvated forms such as, for example, hydrated forms. It is to be understood that the invention encompasses all such solvated forms which inhibit DNA gyrase and/or topoisomerase IV.

There follow particular and suitable values for certain substituents and groups referred to in this specification. These values may be used where appropriate with any of the definitions and embodiments disclosed hereinbefore, or hereinafter. For the avoidance of doubt each stated species represents a particular and independent aspect of this invention.

In one embodiment the invention provides compounds represented by formula (I):

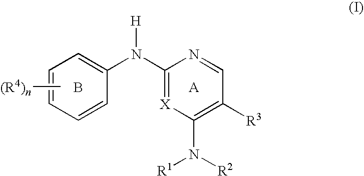

or a pharmaceutically acceptable salt thereof, wherein:

X is CH or N;

R¹ is hydrogen, a C₁₋₆alkyl, C₂₋₆alkenyl, C₂₋₆alkynyl, C₃₋₁₄carbocyclyl, or a heterocyclyl, wherein R¹ may be optionally substituted on carbon by one or more R⁶; and wherein if said hetercyclyl contains an ═N— or a —S— moiety that nitrogen may be optionally substituted by one oxo group and that sulfur may be optionally substituted by one or two oxo groups; and wherein if said heterocyclyl contains an —NH— moiety that nitrogen may be optionally substituted by a group selected from R⁷; provided that R¹ is not a substituted or unsubstituted phenyl;

R² is hydrogen or a C₁₋₆alkyl;

R¹ and R², together with the nitrogen to which they are attached, form a heterocyclyl, wherein said heterocyclyl may be optionally substituted on carbon by one or more R⁸; wherein if said hetercyclyl contains an ═N— or a —S— moiety that nitrogen may be optionally substituted by one oxo group and that sulfur may be optionally substituted by one or two oxo groups; and wherein if said heterocyclyl contains an —NH— moiety that nitrogen may be optionally substituted by a group selected from R⁹;

R³ is a C₆₋₁₄aryl or a heteroaryl; wherein R³ may be optionally substituted on carbon by one or more R¹⁴; and wherein if said heteraryl contains an ═N— or a —S— moiety that nitrogen may be optionally substituted by one oxo group and that sulfur may be optionally substituted by one or two oxo groups; and wherein if said heteroaryl contains an —NH— moiety that nitrogen may be optionally substituted by a group selected from R¹⁵; provided that R³ is not an unsubstituted phenyl or an unsubstituted thiophenyl;

R⁴, for each occurrence, is independently selected from the group consisting of halo, cyano, nitro, hydroxy, C₁₋₆alkyl, C₁₋₆alkoxy, C₁₋₆alkyl-S(O)ₐ—, R¹⁷R¹⁸N—S(O)ₐ—, C₃₋₁₄carbocyclyl, and heterocyclyl; or two R⁴ taken together with the carbon atoms to which they are attached form a C₃₋₁₄carbocyclyl or a heterocyclyl, wherein each R⁴ may be optionally substituted on carbon by one or more R¹⁶; provided that ring B together with —(R⁴)ₙ is not 3,4,5-trimethoxyphenyl;

n is an integer from 1 to 5;

a is 0, 1, or 2;

R⁶, R⁸, and R¹⁴, for each occurrence, are each independently selected from the group consisting of hydroxy, halo, cyano, nitro, C₁₋₆alkyl, C₂₋₆alkenyl, C₂₋₆alkynyl, mercapto, C₁₋₆alkoxy, C₁₋₆alkylS(O)ₐ wherein a is 0 to 2, —C(═N—OH)NH₂, phenoxy, carboxy, amino, N—C₁₋₆alkylamino, N,N—(C₁₋₆alkyl)₂amino, a heterocyclyl, C₁₋₆alkoxycarbonyl, C₁₋₆alkanoyl, C₁₋₆alkanoyloxy, C₁₋₆alkanoylamino, C₁₋₆alkoxycarbonylamino, carbamoyl, N—C₁₋₆alkylcarbamoyl, N,N—(C₁₋₆alkyl)₂carbamoyl, C₁₋₆alkylsulphonylamino, sulphamoyl, N—(C₁₋₆alkyl)sulphamoyl, N,N—(C₁₋₆alkyl)₂sulphamoyl, sulphamoylamino, N—(C₁₋₆alkyl)sulphamoylamino, N,N—(C₁₋₆alkyl)₂sulphamoylamino, C₃₋₁₄carbocyclyl-L- and heterocyclyl-L-; wherein R⁶, R⁸, and R¹⁴ may be each independently optionally substituted on carbon by one or more R¹⁰; and wherein if said hetercyclyl contains an ═N— or a —S— moiety that nitrogen may be optionally substituted by one oxo group and that sulfur may be optionally substituted by one or two oxo groups; and wherein if said heterocyclyl contains an —NH— moiety that nitrogen may be optionally substituted by a group selected from R¹¹;

R⁷, R⁹, and R¹⁵, for each occurrence, are each independently selected from the group consisting of C₁₋₆alkyl, C₁₋₆alkoxycarbonyl, C₁₋₆alkanoyl, carbamoyl, N—C₁₋₆alkylcarbamoyl, N,N—(C₁₋₆alkyl)₂carbamoyl, C₃₋₁₄carbocyclyl-C(O)-, heterocyclyl-C(O)—, (C₁₋₆alkyl)₃silyl, C₁₋₆alkylS(O)ₐ wherein a is 0 to 2, wherein R⁷, R⁹, and R¹⁵ may be each independently optionally substituted on carbon by one or more R¹²; and wherein if said hetercyclyl contains an ═N— or a —S— moiety that nitrogen may be optionally substituted by one oxo group and that sulfur may be optionally substituted by one or two oxo groups; and wherein if said heterocyclyl contains an —NH— moiety that nitrogen may be optionally substituted by a group selected from R¹³;

L, for each occurrence, is independent selected from a direct bond, —O—, —N(R²⁵)—, —C(O)—, —N(R²⁵)C(O)—, —C(O)N(R²⁵)—, —S(O)ₛ—, —SO₂N(R²⁵)— or —N(R²⁵)SO₂—; wherein R²⁵, for each occurrence, is independently selected from hydrogen or C₁₋₆alkyl and s is 0, 1 or 2;

R¹⁰ and R¹², for each occurrence, are independently selected from the group consisting of C₁₋₆alkyl, phenyl, halo, cyano, nitro, carboxy, hydroxy, C₁₋₆alkoxy, C₁₋₆alkoxycarbonyl, amino, N—C₁₋₆alkylamino, N,N—(C₁₋₆alkyl)₂amino, carbamoyl, N—C₁₋₆alkylcarbamoyl, N,N—(C₁₋₆alkyl)₂carbamoyl and N—C₁₋₆alkyloxycarbamoyl; and

R¹¹ and R¹³, for each occurrence, are each independently a C₁₋₆alkyl;

R¹⁶, for each occurrence, is independently, a halo, hydroxy, a C₁₋₆alkyl, or a C₁₋₆alkoxy;

R¹⁷ and R¹⁸, for each occurrence, are independently hydrogen or a C₁₋₆alkyl; or R¹⁷ and R¹⁸, together with the nitrogen to which they are attached form a heterocyclyl;

provided that —NR¹R² is not —NHCH₃, —N(CH₃)₂.

In one embodiment the invention provides compounds represented by formula (I), or a pharmaceutically acceptable salt thereof, wherein X is N.

In another embodiment the invention provides compounds represented by formula (I), or a pharmaceutically acceptable salt thereof, wherein X is CH.

In another embodiment the invention provides compounds represented by formula (I), or a pharmaceutically acceptable salt thereof, wherein R¹ is a C₁₋₆alkyl that is optionally substituted on carbon by one or more R⁶; and R² is hydrogen.

In another embodiment the invention provides compounds represented by formula (I), or a pharmaceutically acceptable salt thereof, wherein R¹ is n-propyl, 3-(N,N-dimethylamino)-propyl, 3-(2-oxo-pyrrolidino)-propyl, 1-acetyl-piperidine-4-yl, 2-morpholino-ethyl, 2-acetamido-ethyl, 3-acetamido-propyl, pyridin-2-ylmethyl, pyridin-3-ylmethyl, pyridin-4-ylmethyl, 2-[(tert-butoxycarbonyl)amino]-ethyl, 2-carbamoyl-ethyl, 2-(pyridin-2-yl)-ethyl, 2-(pyridin-3-yl)-ethyl, 2-(pyridin-4-yl)ethyl, 2-(1,1-dioxo-thiomorpholino)-ethyl, 3-(1,1-dioxo-thiomorpholino)-propyl, 3-morpholino-propyl, 2-methoxyethyl, tetrahydrofuran-2-ylmethyl, 2-(isopropoxy)ethyl, furan-2-ylmethyl, ethoxycarbonylmethyl, phenoxyethyl, 1-(methoxycarbonyl)ethyl, 6-methyl-pyrazin-3-ylmethyl, isopropyl, 3-[(tert-butoxycarbonyl)amino]-propyl, 3-methoxypropyl, 2-(N,N-dimethylamino)-ethyl, 3-(1H-benzimidazol-2-yl)-propyl, 3-[(6-methyl-pyrazin-3-ylcarbonyl)amino]-propyl, 1-methyl-1H-imidazol-5-ylmethyl, 1-methyl-1H-imidazol-4-ylmethyl, tetrahydrofuran-3-yl, 1-methyl-1H-pyrazol-4-ylmethyl, 2-methoxy-1-methoxymethyl-ethyl, 3-amino-propyl, carboxymethyl, 1-carboxy-ethyl, 1H-benzimidazol-2-ylmethyl, 2-(1H-imidazol-4-yl)-ethyl, 2-(1H-benzimidazol-2-yl)-ethyl, 2-(1H-imidazol-1-yl)-ethyl, 2-(1H-pyrazol-1-yl)-ethyl, 2-(1H-pyrazol-4-yl)-ethyl, 2-(4-methyl-thiazole-5-yl)-ethyl, 2-(4-methyl-piperazino)-ethyl, 3-(1H-benzimidazol-2-yl)-propyl, 2-(5-methyl-1H-pyrazol-4-yl)-ethyl, 3-[(methylsulfonyl)amino]-propyl, or [1-(tert-butoxycarbonyl)-1H-benzimidazol-2-yl]methyl.

In another embodiment the invention provides compounds represented by formula (I), or a pharmaceutically acceptable salt thereof, wherein R¹ is a C₁₋₆alkyl which is optionally substituted with amino, carboxy, N,N-dimethylamino, 2-oxo-pyrrolidino, acetamido, pyridin-2-yl, pyridin-3-yl, pyridin-4-yl, (tert-butoxycarbonyl)amino, carbamoyl, methylsulfonylamino, morpholino, 1,1-dioxo-thiomorpholino, methoxy, tetrahydrofuran-2-yl, isopropoxy, furan-2-yl, ethoxycarbonyl, phenoxy, methoxycarbonyl, 6-methyl-pyrazin-3-yl, benzoimidazol-2-yl, [(6-methyl-pyrazin-3-yl)carbonyl]amino, 1H-imidazol-2-yl, 1H-imidazol-1-yl, 1-methyl- 1H-imidazol-2-yl, 1-methyl- 1H-pyrazol-4-yl, 5-methyl-1H-pyrazol-4-yl, 1H-pyrazol-1-yl, 1H-pyrazol-4-yl, 4-methyl-thiazole-5-yl, or 4-methyl-piperazino.

In another embodiment the invention provides compounds represented by formula (I), or a pharmaceutically acceptable salt thereof, wherein R¹ and R², together with the nitrogen to which they are attached, form a heterocyclyl, wherein said heterocyclyl may be optionally substituted on carbon by one or more R⁸; wherein if said hetercyclyl contains an ═N— or a —S— moiety that nitrogen may be optionally substituted by one oxo group and that sulfur may be optionally substituted by one or two oxo groups; and wherein if said heterocyclyl contains an —NH— moiety that nitrogen may be optionally substituted by a group selected from R⁹.

In another embodiment the invention provides compounds represented by formula (I), or a pharmaceutically acceptable salt thereof, wherein R¹ and R², together with the nitrogen to which they are attached, form a 1H-pyrazol-1-yl, wherein said 1H-pyrazol-1-yl may be optionally substituted on carbon by one or more R⁸.

In another embodiment the invention provides compounds represented by formula (I), or a pharmaceutically acceptable salt thereof, wherein R⁸ is independently a C₁₋₆alkyl or a C₃₋₆cycloalkyl wherein said R⁸ is optionally substituted on carbon by one or more halo.

In another embodiment the invention provides compounds represented by formula (I), or a pharmaceutically acceptable salt thereof, wherein R⁸ is independently a C₁₋₃alkyl or a C₃₋₆cycloalkyl wherein said R⁸ is optionally substituted on carbon by one or more fluoro.

In another embodiment the invention provides compounds represented by formula (I), or a pharmaceutically acceptable salt thereof, wherein R¹ and R², together with the nitrogen to which they are attached, form a 1H-pyrazol-1-yl, wherein 1H-pyrazol-1-yl may be optionally substituted on carbon by one or more methyl, cyclopropyl or trifluoromethyl.

In another embodiment the invention provides compounds represented by formula (I), or a pharmaceutically acceptable salt thereof, wherein R¹ and R², together with the nitrogen to which they are attached, form piperidino, 4-hydroxy-piperidino, 3-hydroxymethyl-piperidino, 4-morpholino-piperidino, 4-(N-methyl-carbamoyl)-piperidino, 4-fluoro-piperidino, 4-methoxy-piperidino, 4-acetamido-piperidino, pyrrolidino, 3-hydroxy-pyrrolidino, 2-methyl-pyrrolidino, 2,5-dimethyl-pyrrolidino, azetidine-1-yl, 4-acetamidopiperidino, 3-trifluoromethyl-1H-pyrazol-1-yl, 3-trifluoromethyl-5-methyl-1H-pyrazol-1-yl, 1H-imidazol-1-yl, 4,5-dichloro-1H-imidazol-1-yl, 2-methyl-1H-imidazol-1-yl, 1H-pyrrol-1-yl, morpholino, 2,6-dimethylmorpholino, 3,5-dimethyl-pyrazol-1-yl, 4-(pyridin-4-yl)-1H-pyrazol-1-yl, 4-chloro-1H-pyrazol-1-yl, 4-trifluoromethyl-1H-imidazol-1-yl, 2-methyl-1H-imidazol-1-yl, 1,2,3-2H-triazol-2-yl, 1,2,3-1H-triazol-1-yl, 1,2,3-1H-benzotriazol-1-yl, 1,2,3-2H-benzotriazol-2-yl, 1H-[1,2,3]triazolo[4,5-b]pyridin-1-yl, 2H-[1,2,3]triazolo[4,5-b]pyridin-2-yl, azepan-1-yl, 4-aceto-piperazino, 4-(2-methoxy-ethyl)-piperazino, 4-methyl-piperazino, 4-[(N,N-dimethylamino)carbonyl]-piperazino, 4-(methylsulfonyl)-piperazino, or 4-cyclopropylcarbonyl-piperazino.

In another embodiment the invention provides compounds represented by formula (I), or a pharmaceutically acceptable salt thereof, wherein R¹ and R², together with the nitrogen to which they are attached, form a heterocyclyl selected from 1H-pyrazol-1-yl, 1H-benzotriazol-1-yl, 2H-benzotriazol-2-yl, and 1H-1,2,3-triazol-1-yl, wherein said heterocyclyl may be optionally substituted on carbon by one or more R⁸; wherein if said hetercyclyl contains an ═N— or a —S— moiety that nitrogen may be optionally substituted by one oxo group and that sulfur may be optionally substituted by one or two oxo groups; and wherein if said heterocyclyl contains an —NH— moiety that nitrogen may be optionally substituted by a group selected from R⁹. In one embodiment, R⁸, for each occurrence, is independently selected from hydroxy, hydroxymethyl, morpholino, N-methylcarbamoyl, fluoro, methoxy, methyl, acetamido, trifluoromethyl, chloro, and pyridin-4-yl. In another embodiment, R⁹, for each occurrence, is independently selected from a C₁₋₆alkyl, 2-methoxyethyl, acetyl, N,N-dimethylcarbamoyl, cyclopropylcarbonyl, methylsulfonyl and tert-butoxycarbonyl.

In another embodiment, R¹ is a C₃₋₁₄carbocyclyl; wherein R¹ may be optionally substituted on carbon by one or more R⁶; provided that R¹ is not a substituted or unsubstituted phenyl. In one embodiment, R¹ is cyclohexyl. In another embodiment, R⁶ is hydroxy.

In another embodiment, R¹ is a heterocycyl; wherein R¹ may be optionally substituted on carbon by one or more R⁶; and wherein if said hetercyclyl contains an ═N— or a —S— moiety that nitrogen may be optionally substituted by one oxo group and that sulfur may be optionally substituted by one or two oxo groups; and wherein if said heterocyclyl contains an —NH— moiety that nitrogen may be optionally substituted by a group selected from R⁷; provided that R¹ is not a substituted or unsubstituted phenyl. In one embodiment, R¹ is piperidinyl or tetrahydrofuranyl which may be optionally substituted on carbon by one or more R⁶; and wherein the —NH— moiety of piperidinyl may be optionally t substituted by a group selected from R⁷.

In another embodiment the invention provides compounds represented by formula (I), or a pharmaceutically acceptable salt thereof, wherein R³ is a C₆₋₁₄aryl; wherein R³ may be optionally substituted on carbon by one or more R¹⁴; provided that R³ is not an unsubstituted phenyl.

In another embodiment the invention provides compounds represented by formula (I), or a pharmaceutically acceptable salt thereof, wherein R³ is phenyl substituted on carbon by two R¹⁴ which taken together with the carbon atoms to which they are attached form a C₃₋₁₄carbocyclyl or a heterocyclyl; wherein R¹⁴ may be independently optionally substituted on carbon by one or more R¹⁰; and wherein if said hetercyclyl contains an ═N— or a —S— moiety that nitrogen may be optionally substituted by one oxo group and that sulfur may be optionally substituted by one or two oxo groups; and wherein if said heterocyclyl contains an —NH— moiety that nitrogen may be optionally substituted by a group selected from R¹¹.

In another embodiment the invention provides compounds represented by formula (I), or a pharmaceutically acceptable salt thereof, wherein R³ is a heteroaryl; wherein R³ may be optionally substituted on carbon by one or more R¹⁴; and wherein if said heteraryl contains an ═N— or a —S— moiety that nitrogen may be optionally substituted by one oxo group and that sulfur may be optionally substituted by one or two oxo groups; and wherein if said heteroaryl contains an —NH— moiety that nitrogen may be optionally substituted by a group selected from R¹⁵; provided that R³ is not an unsubstituted thiophenyl.

In another embodiment the invention provides compounds represented by formula (I), or a pharmaceutically acceptable salt thereof, wherein R¹⁴, for each occurrence, is independently selected from methoxycarbonyl, (methylsulfonyl)amino, ethoxycarbonyl, acetyl, amino, 5-oxo-4,5-dihydro-1,2,4-oxadiazolyl, trifluoromethyl, methoxy, (dimethylsulfamoyl)amino, cyano, fluoro, nitro, (E)-2-carboxyethenyl, 2-carboxy-ethyl, carboxy, (E)-2-ethoxycarbonylethenyl, (E)-2-carbamoyl-ethenyl, (E)-2-(N-methylcarbamoyl)-ethenyl, (E)-2-(N-methoxycarbamoyl)-ethenyl, N-methoxycarbamoyl, N-ethyl-carbamoyl, N-benzyl-carbamoyl, N,N-dimethylcarbamoyl, piperidinocarbonyl, 3,3-difluoro-piperidinocarbonyl, or N′-hydroxycarbamimidoyl.

In another embodiment the invention provides compounds represented by formula (I), or a pharmaceutically acceptable salt thereof, wherein R³ is phenyl; wherein R³ is substituted on carbon by one or more R¹⁴. For example, R¹⁴, for each occurrence, is independently selected from methoxycarbonyl, (methylsulfonyl)amino, ethoxycarbonyl, trifluoromethyl, methoxy, (dimethylsulfamoyl)amino, cyano, fluoro, nitro, (E)-2-carboxyethenyl, 2-carboxy-ethyl, carboxy, (E)-2-ethoxycarbonylethenyl, (E)-2-carbamoyl-ethenyl, (E)-2-(N-methylcarbamoyl)-ethenyl, (E)-2-(N-methoxycarbamoyl)-ethenyl, N-methoxycarbamoyl, N-ethyl-carbamoyl, N-benzyl-carbamoyl, or N,N-dimethylcarbamoyl.

In another embodiment the invention provides compounds represented by formula (I), or a pharmaceutically acceptable salt thereof, wherein R³ is pyrimidinyl, indolinyl, pyridinyl, benzofuranyl, benzothiophenyl, thiophenyl, 1H-pyrazolyl, 4-oxo-1,4-dihydroquinolinyl, thiazolyl, quinolinyl, and benzimidazolyl; wherein R³ may be optionally substituted on carbon by one or more R¹⁴; and wherein if R³ contains an ═N— or a —S— moiety that nitrogen may be optionally substituted by one oxo group and that sulfur may be optionally substituted by one or two oxo groups; and wherein if R³ contains an —NH— moiety that nitrogen may be optionally substituted by a group selected from R¹⁵; provided that R³ is not an unsubstituted thiophenyl. In one embodiment, R¹⁴, for each occurrence, is independently selected from methoxycarbonyl, ethoxycarbonyl, acetyl, amino, 5-oxo-4,5-dihydro-1,2,4-oxadiazolyl, methoxy, carboxy, N-ethyl-carbamoyl, N-benzyl-carbamoyl, N,N-dimethylcarbamoyl, piperidinocarbonyl, 3,3-difluoro-piperidinocarbonyl, or N′-hydroxycarbamimidoyl. In another embodiment, R¹⁵, for each occurrence, is independently selected from tert-butyl-dimethyl-silyl, 2-methoxyethyl, or tert-butoxycarbonyl.

In another embodiment the invention provides compounds represented by formula (I), or a pharmaceutically acceptable salt thereof, wherein R³ is 2-methoxyphenyl, 3-methoxyphenyl, 4-methoxyphenyl, 3-(methoxycarbonyl)-phenyl, 4-(ethoxycarbonyl)-phenyl, 3-[(methylsulfony)amino]-phenyl, 4-methoxy-3-trifluoromethyl, 3,4,5-trimethoxy-phenyl, 3-[(dimethylsulfamoyl)amino]-phenyl, 3-cyano-4-fluoro-phenyl, 3-nitrophenyl, 4-carboxyphenyl, 3-carboxyphenyl, 4-(2-carboxyethyl)-phenyl, 4-[(E)-2-carboxyethenyl]-phenyl, 3-[(E)-2-carboxyethenyl]-phenyl, 3-[(E)-2-ethoxycarbonylethenyl]-phenyl, 3-[(E)-2-(N-methylcarbamoyl)ethenyl]-phenyl, 3-[(E)-2-carbamoylethenyl]-phenyl, 3-[(E)-2-(N-methyloxycarbamoyl)ethenyl]-phenyl, 3-(N-ethylcarbamoyl)-phenyl, 3-(N-benzylcarbamoyl)-phenyl, 4-(N-ethylcarbamoyl)-phenyl, 4-(N,N-dimethylcarbamoyl)-phenyl, or 4-(N-benzylcarbamoyl)-phenyl.

In another embodiment the invention provides compounds represented by formula (I), or a pharmaceutically acceptable salt thereof, wherein R³ is 2-methoxypyrimidin-5-yl, indolin-6-yl, 5-ethoxycarbonyl-pyridin-3-yl, 2,6-dimethoxypyridin-4-yl, benzofuran-2-yl, 5-acetyl-thiophen-2-yl, 5-cyano-pyridin-3-yl, 1-(tert-butoxy-dimethyl-silyl)-1H-indolin-3-yl, 5-carboxy-thiophen-2-yl, 6-methoxy-pyridin-3-yl, 2-amino-pyrimidin-5-yl, 1H-pyrazol-4-yl, 6-amino-pyridin-3-yl, 2-methoxycarbonyl-benzothiophen-5-yl, 2-carboxy-benzothiophen-5-yl, pyridin-3-yl, pyrimidin-5-yl, 5-carboxy-pyridin-3-yl, 5-(5-oxo-4,5-dihydro-1,2,4-oxadiazol-3-yl)-pyridin-3-yl, 4-oxo-3-ethoxycarbonyl-1-(2-methoxyethyl)-1,4-dihydroquinolin-6-yl, 4-oxo-3-carboxy-1-(2-methoxyethyl)-1,4-dihydroquinolin-6-yl, 4-methoxy-thiazol-2-yl, 3-carboxy-quinolin-6-yl, 5-(N,N-dimethylcarbamoyl)-thiophen-2-yl, 5-(N-methylcarbamoyl)-thiophen-2-yl, 5-(N-benzylcarbamoyl)-thiophen-2-yl, 5-(piperdinocarbonyl)-thiophen-2-yl, 5-(3,3-difluoropiperdinocarbonyl)-thiophen-2-yl, 5-(N-benzylcarbamoyl)-pyridin-3-yl, 5-(N′-hydroxycarbamimidoyl)-pyridin-3-yl, or 5-(5-oxo-4,5-dihydro-1,2,4-oxadiazol-3-yl)-pyridin-3-yl.

In another embodiment the invention provides compounds represented by formula (I), or a pharmaceutically acceptable salt thereof, wherein R³ is 1-ethyl -(2-methoxyethyl)-4-oxo-1,4-dihydroquinolin-6-yl-carboxylate or 1H-indol-6-yl.

In another embodiment the invention provides compounds represented by formula (I), or a pharmaceutically acceptable salt thereof, wherein R³ is a pyridin-3-yl or 2-oxo-pyridin-5-yl group wherein said pyridin-3-yl or 2-oxo-pyridin-5-yl group may be optionally substituted on carbon by one or more R¹⁴ and wherein the N of said 2-oxo-pyridin-5-yl is substituted by a group selected from R¹⁵. In another embodiment the invention provides compounds represented by formula (I), or a pharmaceutically acceptable salt thereof, wherein R¹⁴, for each occurrence, is independently a carboxy, C₁₋₆alkoxy, C₁₋₆alkylsulphonylcarbamoyl, C₁₋₆alkoxycarbamoyl, or C₁₋₆alkylS(O)ₐ wherein a is 0, 1 or 2.

In another embodiment the invention provides compounds represented by formula (I), or a pharmaceutically acceptable salt thereof, wherein R¹⁵, for each occurrence, is independently a C₁₋₆alkyl wherein said C₁₋₆alkyl is optionally substituted by C₁₋₆alkoxy or saturated heterocyclyl.

In another embodiment the invention provides compounds represented by formula (I), or a pharmaceutically acceptable salt thereof, wherein R¹⁴, for each occurrence, is independently a carboxy, C₁₋₃alkoxy, C₁₋₃alkylsulphonylcarbamoyl, C₁₋₃alkoxycarbamoyl, or C₁₋₃alkylS(O)ₐ wherein a is 0, 1 or 2.

In another embodiment the invention provides compounds represented by formula (I), or a pharmaceutically acceptable salt thereof, wherein R¹⁵, for each occurrence, is independently a C₁₋₃alkyl wherein said C₁₋₃alkyl is optionally substituted by C₁₋₃alkoxy or saturated heterocyclyl.

In another embodiment the invention provides compounds represented by formula (I), or a pharmaceutically acceptable salt thereof, wherein R¹⁵, for each occurrence, is independently a C₁₋₃alkyl wherein said C₁₋₃alkyl is optionally substituted by C₁₋₃alkoxy or morpholino.

In another embodiment the invention provides compounds represented by formula (I), or a pharmaceutically acceptable salt thereof, wherein R³ is a 6-membered heteroaryl containing at least one nitrogen atom wherein one of the carbon atoms of said 6-membered heteroaryl ring may be optionally substituted with O to form a —(CO)—, and further wherein said 6-membered heteroaryl may be optionally substituted on carbon by one or more R¹⁴ and when one of the carbon atoms of said 6-membered heteroaryl ring is substituted with O to form a —(CO)—, the nitrogen of that 6-membered heteroaryl is substituted by a group selected from R¹⁵.

In another embodiment the invention provides compounds represented by formula (I), or a pharmaceutically acceptable salt thereof, wherein R³ is 2-oxo-3-carboxy-1-ethyl-pyridin-5-yl, 2-oxo-3-carboxy-1-(2-methoxyethyl)-pyridin-5-yl, 3-carboxy-6-(2-dimethylaminoethoxy)-pyridin-5-yl, 3-(N-2-hydroxyethylcarbamoyl)-pyridin-5-yl, 3-N-(2-methylsulfonylethyl)carbamoyl-pyridin-5-yl, 2-methoxy-3-carboxy-pyridin-5-yl, 3-N-methylcarbamoyl-pyridin-5-yl, 2-oxo-3-carboxy-1-methyl-pyridin-5-yl, 2-oxo-3-N-(methylsulfonyl)-carbamoyl-1-methyl-pyridin-5-yl, 3-N-methoxycarbamoyl-pyridin-5-yl, 3-carboxy-pyridin-5-yl, 2-oxo-3-carboxy-1-(2-morpholinoethyl)-pyridin-5-yl, 3-carboxy-2-methylsulphanyl-pyridin-5-yl, 3-carboxy-pyridin-5-yl, 2-methoxy-3-(N-methylsulfonylcarbamoyl)-pyridin-5-yl, and 2-methoxy-3-(N-ethylsulfonylcarbamoyl)-pyridin-5-yl.

In another embodiment the invention provides compounds represented by formula (I), or a pharmaceutically acceptable salt thereof, wherein n is 1.

In another embodiment the invention provides compounds represented by formula (I), or a pharmaceutically acceptable salt thereof, wherein n is 2.

In another embodiment the invention provides compounds represented by formula (I), or a pharmaceutically acceptable salt thereof, wherein n is 3.

In another embodiment the invention provides compounds represented by formula (I), or a pharmaceutically acceptable salt thereof, wherein n is 4.

In another embodiment the invention provides compounds represented by formula (I), or a pharmaceutically acceptable salt thereof, wherein n is 4.

In another embodiment the invention provides compounds represented by formula (I), or a pharmaceutically acceptable salt thereof, wherein R⁴, for each occurrence, is independently a halo, C₁₋₆alkyl or C₁₋₆alkoxy.

In another embodiment the invention provides compounds represented by formula (I), or a pharmaceutically acceptable salt thereof, wherein n is 2 and R⁴, for each occurrence, is independently a halo, C₁₋₆alkyl or C₁₋₆alkoxy. In another embodiment the invention provides compounds represented by formula (I), or a pharmaceutically acceptable salt thereof, wherein n is 2 and R⁴, for each occurrence, is independently a F, Cl, methyl or methoxy.

In another embodiment the invention provides compounds represented by formula (I), or a pharmaceutically acceptable salt thereof, wherein R⁴, for each occurrence, is independently selected from methyl, hydroxymethyl, fluoro, chloro, bromo, 1H-tetrazole-1-yl, methoxy, cyano, 5-methyl-1H-tetrazole-1-yl, 2-methoxyethoxy, nitro, morpholinosulfonyl, or trifluoromethyl.

In another embodiment the invention provides compounds represented by formula (I), or a pharmaceutically acceptable salt thereof, wherein n is 2 and one R⁴ is fluoro and the other is chloro.

In another embodiment the invention provides compounds represented by formula (I), or a pharmaceutically acceptable salt thereof, wherein two adjacent R⁴ together with ring B form 1H-indolinyl.

In another embodiment the invention provides compounds represented by formula (I), or a pharmaceutically acceptable salt thereof, wherein:

X is N;

R¹ is a C₁₋₆alkyl which is optionally substituted with on carbon with one or more R⁶;

R² is hydrogen;

R³ is 5-carboxy-pyridin-3-yl, 5-ethoxycarbonyl-pyridin-3-yl, 3-[(E)-2-carboxyethenyl]-phenyl, 3-[(E)-2-ethoxycarbonylethenyl]-phenyl, 3-[(E)-2-(N-methylcarbamoyl)ethenyl]-phenyl, or 3-[(E)-2-carbamoylethenyl]-phenyl;

n is 2; and

R⁴, for each occurrence is independently selected from a halo.

In another embodiment the invention provides compounds represented by formula (I), or a pharmaceutically acceptable salt thereof, wherein:

X is N;

R¹ is n-propyl, 3-(N,N-dimethylamino)-propyl, 2-(pyridin-2-yl)-ethyl, 2-(pyridin-3-yl)-ethyl, or 2-(pyridin-4-yl)ethyl;

R² is hydrogen;

R³ is 5-carboxy-pyridin-3-yl, 5-ethoxycarbonyl-pyridin-3-yl, 3-[(E)-2-carboxyethenyl]-phenyl, 3-[(E)-2-ethoxycarbonylethenyl]-phenyl, 3-[(E)-2-(N-methylcarbamoyl)ethenyl]-phenyl, or 3-[(E)-2-carbamoylethenyl]-phenyl;

n is 2; and

one of R⁴ is fluoro and the other is chloro.

In another embodiment the invention provides compounds represented by formula (I), or a pharmaceutically acceptable salt thereof, wherein:

X is N;

R¹ and R², together with the nitrogen to which they are attached, form pyrazol-1-yl wherein said pyrazol-1-yl may be optionally substituted on carbon by one or more R⁸;

R³ is a 6-membered heteroaryl containing at least one nitrogen atom wherein one of the carbon atoms of said 6-membered heteroaryl ring may be optionally substituted with O to form a —(CO)—, and further wherein said 6-membered heteroaryl may be optionally substituted on carbon by one or more R¹⁴ and when one of the carbon atoms of said 6-membered heteroaryl ring is substituted with O to form a —(CO)—, the nitrogen of that 6-membered heteroaryl is substituted by a group selected from R¹⁵;

n is 2;

R⁴, for each occurrence, is independently a halo, C₁₋₆alkyl or C₁₋₆alkoxy;

R⁸, for each occurrence, is independently a C₁₋₆alkyl or a C₃₋₆cycloalkyl wherein said R⁸ is optionally substituted on carbon by one or more fluoro;

R¹⁴, for each occurrence, is independently a carboxy, C₁₋₆alkoxy, C₁₋₃alkylsulphonylcarbamoyl, N—C₁₋₃alkylcarbamoyl, N—C₁₋₃alkoxycarbamoyl, or C₁₋₆alkylS(O)ₐ wherein a is 0, 1 or 2 wherein said R¹⁴ may be optionally substituted on carbon by one or more hydroxy, (C₁₋₃alkyl)₂N, or C₁₋₃alkylsulfonyl; and

R¹⁵, for each occurrence, is independently a C₁₋₆alkyl wherein said C₁₋₆alkyl is optionally substituted by C₁₋₆alkoxy or saturated heterocyclyl.

In another embodiment the invention provides compounds represented by formula (I), or a pharmaceutically acceptable salt thereof, wherein:

X is N;

R¹ and R², together with the nitrogen to which they are attached, form pyrazol-1-yl wherein said pyrazol-1-yl may be optionally substituted on carbon by one or more R⁸;

R³ is a 6-membered heteroaryl containing at least one nitrogen atom wherein one of the carbon atoms of said 6-membered heteroaryl ring may be optionally substituted with O to form a —(CO)—, and further wherein said 6-membered heteroaryl may be optionally substituted on carbon by one or more R¹⁴ and when one of the carbon atoms of said 6-membered heteroaryl ring is substituted with O to form a —(CO)—, the nitrogen of that 6-membered heteroaryl is substituted by a group selected from R¹⁵;

n is 2;

R⁴, for each occurrence, is independently a halo, C₁₋₆alkyl or C₁₋₆alkoxy;

R⁸, for each occurrence, is independently a methyl, trifluoromethyl or a cyclopropyl;

R¹⁴, for each occurrence, is independently a carboxy, C₁₋₆alkoxy, C₁₋₃alkylsulphonylcarbamoyl, N—C₁₋₃alkylcarbamoyl, N—C₁₋₃alkoxycarbamoyl, or C₁₋₆alkylS(O)ₐ wherein a is 0, 1 or 2 wherein said R¹⁴ may be optionally substituted on carbon by one or more hydroxy, (C₁₋₃alkyl)₂N, or C₁₋₃alkylsulfonyl; and

R¹⁵, for each occurrence, is independently a C₁₋₆alkyl wherein said C₁₋₆alkyl is optionally substituted by C₁₋₆alkoxy or saturated heterocyclyl.

In another embodiment the invention provides compounds represented by formula (I), or a pharmaceutically acceptable salt thereof, wherein:

X is N;

R¹ and R², together with the nitrogen to which they are attached, form pyrazol-1-yl wherein said pyrazol-1-yl may be optionally substituted on carbon by one or more R⁸;

R³ is a pyridin-3-yl or 2-oxo-pyridin-5-yl group wherein said pyridin-3-yl or 2-oxo-pyridin-5-yl group may be optionally substituted on carbon by one or more R¹⁴ and wherein the N of said 2-oxo-pyridin-5-yl is substituted by a group selected from R¹⁵;

n is 2;

R⁴, for each occurrence, is independently a halo, C₁₋₃alkyl or C₁₋₃alkoxy;

R⁸, for each occurrence, is independently a C₁₋₃alkyl optionally substituted on carbon by one or more fluoro;

R¹⁴, for each occurrence, is independently a carboxy, C₁₋₃alkoxy, C₁₋₃alkylsulphonylcarbamoyl, N—C₁₋₃alkylcarbamoyl, N—C₁₋₃alkoxycarbamoyl, or C₁₋₃alkylS(O)ₐ wherein a is 0, 1 or 2, wherein said R¹⁴ may be optionally substituted on carbon by one or more hydroxy, (C₁₋₃alkyl)₂N—, or ₁₋₃alkylsulfonyl;

R¹⁵, for each occurrence, is independently a C₁₋₃alkyl wherein said C₁₋₃alkyl is optionally substituted by C₁₋₃alkoxy or saturated heterocyclyl.

In another embodiment the invention provides compounds represented by formula (I), or a pharmaceutically acceptable salt thereof, wherein:

X is N;

R¹ and R², together with the nitrogen to which they are attached, form a heterocyclyl selected from 1H-pyrazol-1-yl, 1H-benzotriazol-1-yl, 2H-benzotriazol-2-yl, and 1H-1,2,3-triazol-1-yl, wherein said heterocyclyl may be optionally substituted on carbon by one or more R⁸; wherein if said hetercyclyl contains an ═N— or a —S— moiety that nitrogen may be optionally substituted by one oxo group and that sulfur may be optionally substituted by one or two oxo groups; and wherein if said heterocyclyl contains an —NH— moiety that nitrogen may be optionally substituted by a group selected from R⁹;

R³ is 5-carboxy-pyridin-3-yl, 5-ethoxycarbonyl-pyridin-3-yl, 3-[(E)-2-carboxyethenyl]-phenyl, 3-[(E)-2-ethoxycarbonylethenyl]-phenyl, 3-[(E)-2-(N-methylcarbamoyl)ethenyl]-phenyl, or 3-1(E)-2-carbamoylethenyl]-phenyl;

n is 2;

R⁴, for each occurrence is independently a halo;

R⁸, for each occurrence, is independently hydroxy, hydroxymethyl, morpholino, N-methylcarbamoyl, fluoro, methoxy, methyl, acetamido, trifluoromethyl, chloro, or pyridin-4-yl;

R⁹, for each occurrence, is independently a C₁₋₆alkyl, 2-methoxyethyl, acetyl, N,N-dimethylcarbamoyl, cyclopropylcarbonyl, methylsulfonyl or tert-butoxycarbonyl.

Particular compounds of the invention are the compounds of the Examples, and pharmaceutically acceptable salts thereof, each of which provides a further independent aspect of the invention. For those examples which are themselves in the form of a salt, a further independent aspect of the invention is those specific salts as well as other pharmaceutically acceptable salts thereof and the free bases thereof. In further aspects, the present invention also comprises any two or more compounds of the Examples.

In another embodiment the invention provides compounds of Examples 319, 675, 677, 679, 681, 683, 684, 761, 815, 854, 861, 863, 909, 918, 919, 1019, 1026, 1075, 1076, 1086, 1087, 1088, 1143, 1145, 1152, 1159 and 1160, and or a pharmaceutically acceptable salt thereof.

In another embodiment the invention provides compounds of Examples 319, 638, 675, 677, 679, 681, 683, 684, 761, 815, 854, 861, 863, 909, 918, 919, 1019, 1026, 1075, 1076, 1086, 1087, 1088, 1143, 1145, 1152, 1159 and 1160, and or a pharmaceutically acceptable salt thereof.

In another embodiment, the invention provides pharmaceutical compositions comprising a pharmaceutically acceptable excipient or carrier and a compound represented by formula (I), or a pharmaceutically acceptable salt thereof.

In a further aspect the present invention provides a process for preparing a compound of formula (I), or a pharmaceutically-acceptable salt thereof, wherein variable groups in the schemes below are as defined in formula (I) unless otherwise specified. In general, the compounds of the invention can be prepared by adding Ring B, —NR¹R² and R³ to a pyrimidine or pyridine core in any order. For example, formula (I) can be prepared by the following methods:

### Process a: Reacting a Compound of Formula (i):

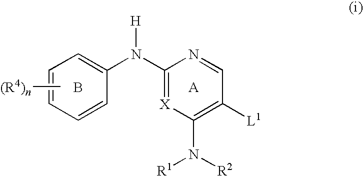

with a compound of formula (ii):

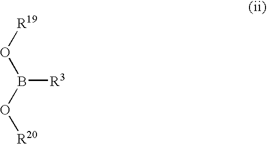

in the presence of a palladium(O) catalyst and a base, such as sodium carbonate, wherein L¹ is a displaceable group such as a halo; and R¹⁹ and R²⁰ are each independently hydrogen or a C₁₋₆alkyl; or R¹⁹ and R²⁰ together form a C₂₋₄alkylene bridge which may be optionally substituted with one or more independently selected C₁₋₄alkyl groups.
Process B: Reacting a Compound of Formula (iii):

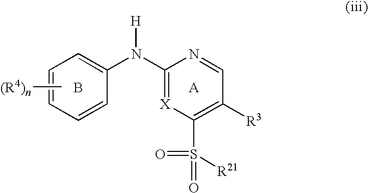

wherein R²¹ is a C₁₋₆alkyl or a C₆₋₁₄aryl;

with a compound represented by formula (iv):

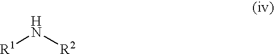

in the presence of a base, such as NaH, diisopropylethylamine, or NaOH. In some instances, it may be necessary to heat the reaction.

Compounds represented by formula (i) can be prepared by reacting a compound represented by formula (v):

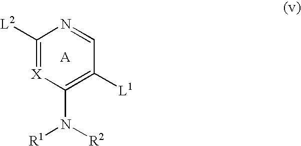

with a compound represented by formula (vi):

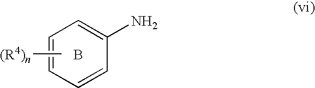

in the presence of an acid, such as HCl and heat, wherein L¹ and L² are each, independently, displaceable groups, such as a halo.

A compound represented by formula (v) can be prepared from a pyrimidine or pyridine derivative by reacting a compound represented by formula (vii):

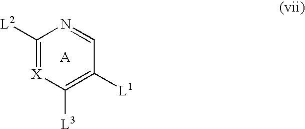

with a compound represented by formula (iv) in the presence of a base and optionally heat, wherein L¹, L² and L³ are each, independently, displaceable groups, such as a halo.

Compounds represented by formula (iii) can be prepared by treating a compound represented by formula (viii):

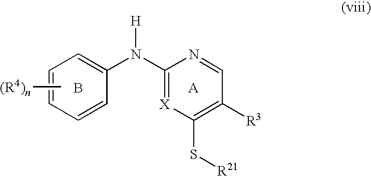

with a peroxide, such as 3-chloroperoxybenzoic acid.

Compounds represented by formula (viii) can be prepared by reacting a compound represented by formula (ix):

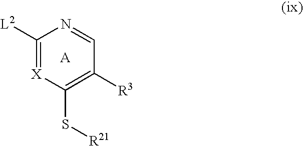

with a compound represented by formula (vi) in the presence of an acid, such as HCl and heat.

Compounds represented by formula (ix) can be prepared by reacting a compound represented by formula (x):

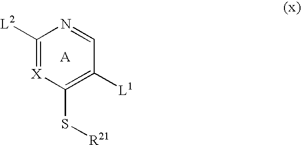

with a compound represented by formula (ii) in the presence of a palladium(O) catalyst and a base, such as sodium carbonate.

Alternatively, compounds represented by formula (iii) can be prepared by reacting a compound represented by formula (xi):

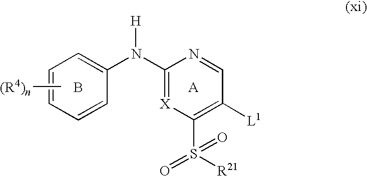

with a compound of formula (ii) in the presence of a palladium(O) catalyst and a base, such as sodium carbonate.

Compounds represented by formula (xi) can be prepared by treating a compound represented by formula (xii):

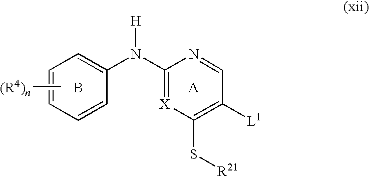

with a peroxide, such as 3-chloroperoxybenzoic acid.

Compounds represented by formula (xii) can be prepared by reacting a compound represented by formula (x) with an aniline derivative represented by formula (vi) in the presence of an acid, such as HCl and heat.

Compounds represented by formulas (ii), (iv), (vi), (vii) and (x) can be purchased or prepared by standard methods known in the art.

The formation of a pharmaceutically-acceptable salt is within the skill of an ordinary organic chemist using standard techniques.

It will be appreciated that certain of the various ring substituents in the compounds of the present invention may be introduced by standard aromatic substitution reactions or generated by conventional functional group modifications either prior to or immediately following the processes mentioned above, and as such are included in the process aspect of the invention. The reagents used to introduce such ring substituents are either commercially available or are made by processes known in the art.

Introduction of substituents into a ring may convert one compound of the formula (I) into another compound of the formula (I). Such reactions and modifications include, for example, introduction of a substituent by means of an aromatic substitution reaction, reduction of substituents, alkylation of substituents, oxidation of substituents, esterification of substituents, amidation of substituents, formation of heteroaryl rings. Particular examples of aromatic substitution reactions include the introduction of alkoxides, diazotization reactions followed by introduction of thiol group, alcohol group, halogen group. Examples of modifications include; oxidation of alkylthio to alkylsulphinyl or alkylsulphonyl. (See Advanced Organic Chemistry, 4^{th} Edition, by Jerry March, published by John Wiley & Sons 1992). In one embodiment, an ester substituent on a compound of formula (I) may be converted to a carboxylic acid by treating the ester with a base, such as sodium hydroxide, barium hydroxide, or trimethyltin hydroxide. In another embodiment, a carboxylic acid substituent on a compound of formula (I) may be converted to an amide by reacting the carboxylic acid group with a primary or secondary amine in the presence of a peptide coupling reagent, such as O-(7-azabenzotriazol-1-yl)-N,N,N′,N′-tetramethyluronium hexafluorophosphate (HATU), dicyclohexylcarbodiimide (DCC), or 1-ethyl-3-(3-dimethyllaminopropyl)carbodiimide (EDC). The reagents and reaction conditions for such procedures are well known in the chemical art.

The skilled organic chemist will be able to use and adapt the information contained and referenced within the above references, and accompanying Examples therein and also the Examples herein, to obtain necessary starting materials, and products. If not commercially available, the necessary starting materials for the procedures such as those described above may be made by procedures which are selected from standard organic chemical techniques, techniques which are analogous to the synthesis of known, structurally similar compounds, or techniques which are analogous to the above described procedure or the procedures described in the examples. It is noted that many of the starting materials for synthetic methods as described above are commercially available and/or widely reported in the scientific literature, or could be made from commercially available compounds using adaptations of processes reported in the scientific literature. The reader is further referred to Advanced Organic Chemistry, 4^{th} Edition, by Jerry March, published by John Wiley & Sons 1992, for general guidance on reaction conditions and reagents.

It will also be appreciated that in some of the reactions mentioned herein it may be necessary/desirable to protect any sensitive groups in compounds. The instances where protection is necessary or desirable are known to those skilled in the art, as are suitable methods for such protection. Conventional protecting groups may be used in accordance with standard practice (for illustration see T. W. Greene, Protective Groups in Organic Synthesis, John Wiley and Sons, 1991).

Examples of a suitable protecting group for a hydroxy group is, for example, an acyl group, for example an alkanoyl group such as acetyl, an aroyl group, for example benzoyl, a silyl group such as trimethylsilyl or an arylmethyl group, for example benzyl. The deprotection conditions for the above protecting groups will necessarily vary with the choice of protecting group. Thus, for example, an acyl group such as an alkanoyl or an aroyl group may be removed, for example, by hydrolysis with a suitable base such as an alkali metal hydroxide, for example lithium or sodium hydroxide. Alternatively a silyl group such as trimethylsilyl may be removed, for example, by fluoride or by aqueous acid; or an arylmethyl group such as a benzyl group may be removed, for example, by hydrogenation in the presence of a catalyst such as palladium-on-carbon.

A suitable protecting group for an amino group is, for example, an acyl group, for example an alkanoyl group such as acetyl, an alkoxycarbonyl group, for example a methoxycarbonyl, ethoxycarbonyl or t-butoxycarbonyl group, an arylmethoxycarbonyl group, for example benzyloxycarbonyl, or an aroyl group, for example benzoyl. The deprotection conditions for the above protecting groups necessarily vary with the choice of protecting group. Thus, for example, an acyl group such as an alkanoyl or alkoxycarbonyl group or an aroyl group may be removed for example, by hydrolysis with a suitable base such as an alkali metal hydroxide, for example lithium or sodium hydroxide. Alternatively an acyl group such as a t-butoxycarbonyl group may be removed, for example, by treatment with a suitable acid as hydrochloric, sulphuric or phosphoric acid or trifluoroacetic acid and an arylmethoxycarbonyl group such as a benzyloxycarbonyl group may be removed, for example, by hydrogenation over a catalyst such as palladium-on-carbon, or by treatment with a Lewis acid for example boron tris(trifluoroacetate). A suitable alternative protecting group for a primary amino group is, for example, a phthaloyl group which may be removed by treatment with an alkylamine, for example dimethylaminopropylamine or 2-hydroxyethylamine, or with hydrazine.

A suitable protecting group for a carboxy group is, for example, an esterifying group, for example a methyl or an ethyl group which may be removed, for example, by hydrolysis with a base such as sodium hydroxide, or for example a t-butyl group which may be removed, for example, by treatment with an acid, for example an organic acid such as trifluoroacetic acid, or for example a benzyl group which may be removed, for example, by hydrogenation over a catalyst such as palladium-on-carbon, or for example, an allyl group which may be removed, for example, by use of a palladium catalyst such as palladium acetate.

The protecting groups may be removed at any convenient stage in the synthesis using conventional techniques well known in the chemical art, or they may be removed during a later reaction step or work-up.

When an optically active form of a compound of the invention is required, it may be obtained by carrying out one of the above procedures using an optically active starting material (formed, for example, by asymmetric induction of a suitable reaction step), or by resolution of a racemic form of the compound or intermediate using a standard procedure, or by chromatographic separation of diastereoisomers (when produced). Enzymatic techniques may also be useful for the preparation of optically active compounds and/or intermediates.

Similarly, when a pure regioisomer of a compound of the invention is required, it may be obtained by carrying out one of the above procedures using a pure regioisomer as a starting material, or by resolution of a mixture of the regioisomers or intermediates using a standard procedure.

### Enzyme Potency Testing Methods

E.coli GyrB ATPase Inhibition Activity: Compounds may be tested for inhibition of E. coli GyrB ATPase activity using an ammonium molybdate/malachite green-based phosphate detection assay (Lanzetta, P. A., L. J. Alvarez, P. S. Reinach, and O. A. Candia, 1979, 100: 95-97). Assays are performed in multiwell plates in 30 μl reactions containing: 50 mM Hepes buffer pH 7.5, 75 mM ammonium acetate, 8.0 mM magnesium chloride, 0.5 mM ethylenediaminetetraacetic acid, 5% glycerol, 1 mM 1,4-Dithio-DL-threitol, 200 nM bovine serum albumin, 1.6 μg/ml sheared salmon sperm DNA, 400 pM E. coli GyrA, 400 pM E. coli GyrB, 250 μM ATP, and the test compound in dimethylsulfoxide. Reactions are quenched with 30 μl of ammonium molybdate/malachite green detection reagent containing 1.2 mM malachite green hydrochloride, 8.5 mM ammonium molybdate tetrahydrate, and 1 M hydrochloric acid. Plates can be read in an absorbance plate reader at 650 nm and percent inhibition values are calculated using dimethylsulfoxide (2%)-containing reactions as 0% inhibition and EDTA-containing (2.4 μM) reactions as 100% inhibition controls. An IC₅₀ measurement of compound potency for each compound can be determined from reactions performed in the presence of 10 different compound concentrations.

E. coli Topoisomerase IV ATPase Inhibition Activity: Compounds may be tested for inhibition of E. coli topoisomerase IV ATPase activity as described above for E. coli GyrB except the 30 μl reactions contained the following: 20 mM TRIS buffer pH 8, 50 mM ammonium acetate, 8 mM magnesium chloride, 5% glycerol, 5 mM 1,4-Dithio-DL-threitol, 0.005% Brij-35, 5 μg/ml sheared salmon sperm DNA, 500 pM E. coli ParC, 500 pM E. coli ParE, 160 μM ATP, and test compound in dimethylsulfoxide. An IC₅₀ measurement of compound potency for each compound can be determined from reactions performed in the presence of 10 different compound concentrations.

S. aureus GyrB ATPase Inhibition Activity: Compounds may be tested for inhibition of S. aureus GyrB ATPase activity using an ammonium molybdate/malachite green-based phosphate detection assay (Lanzetta, P. A., L. J. Alvarez, P. S. Reinach, and O. A. Candia, 1979, 100: 95-97). Assays are performed in multiwell plates in 30 μl reactions containing: 50 mM Hepes buffer pH 7.5, 75 mM ammonium acetate, 8.0 mM magnesium chloride, 0.5 mM ethylenediaminetetraacetic acid, 5% glycerol, 1.0 mM 1,4-Dithio-DL-threitol, 200 nM bovine serum albumin, 1.0 μg/ml sheared salmon sperm DNA, 250 pM E. coli GyrA, 250 pM S. aureus GyrB, 250 μM ATP, and test compound in dimethylsulfoxide. Reactions are quenched with 30 μl of ammonium molybdate/malachite green detection reagent containing 1.2 mM malachite green hydrochloride, 8.5 mM ammonium molybdate tetrahydrate, and 1 M hydrochloric acid. Plates are read in an absorbance plate reader at 650 nm and percent inhibition values can be calculated using dimethylsulfoxide (2%)-containing reactions as 0% inhibition and EDTA-containing (2.4 μM) reactions as 100% inhibition controls. An IC₅₀ measurement of compound potency for each compound can be determined from reactions performed in the presence of 10 different compound concentrations.

S. pneumoniae Topoisomerase IV ATPase Inhibition Activity: Compounds may be tested for inhibition of S. pneumoniae ParE ATPase activity using an ammonium molybdate/malachite green-based phosphate detection assay (Lanzetta, P. A., L. J. Alvarez, P. S. Reinach, and O. A. Candia, 1979, 100: 95-97). Assays are performed in multiwell plates in 30 μl reactions containing: 20 mM Tris buffer pH 8.0, 50 mM ammonium acetate, 8.0 mM magnesium chloride, 0.5 mM ethylenediaminetetraacetic acid, 5% glycerol, 5 mM 1,4-Dithio-DL-threitol, 0.005% Brij-35, 5 μg/ml sheared salmon sperm DNA, 1.25 nM S. pneumoniae ParE, 160 μM ATP, and test compound in dimethylsulfoxide. Reactions are quenched with 30 μl of ammonium molybdate/malachite green detection reagent containing 1.2 mM malachite green hydrochloride, 8.5 mM ammonium molybdate tetrahydrate, and 1 M hydrochloric acid. Plates are read in an absorbance plate reader at 650 nm and percent inhibition values are calculated using dimethylsulfoxide (2%)-containing reactions as 0% inhibition and EDTA-containing (20 μM) reactions as 100% inhibition controls. An IC₅₀ measurement of compound potency for each compound can be determined from reactions performed in the presence of 10 different compound concentrations.

Many of the compounds of the invention were tested in an assay substantially similar to the assays described above for measuring the inhibition of E.coli GyrB ATPase, E. coli Topoisomerase IV ATPase, S. aureus GyrB ATPase, and S. pneumoniae Topoisomerase IV ATPase, and had IC50 values of <200 μM in one or more assays.

The compounds of the examples (Ex) were tested in an assay substantially similar to the assay described above for measuring the inhibition of S. pneumoniae Topoisomerase IV ATPase and were found to have a percent inhibition (% Inh) of S. pneumoniae Topoisomerase IV ATPase as shown in the table below.

| | % Inh |
|---|---|
| Ex | (13 μM) |
| 1 | 5.14 |
| 2 | 77.79 |
| 3 | 56.76 |
| 4 | 93.06 |
| 5 | 103.92 |
| 6 | 101.54 |
| 7 | 98.67 |
| 8 | 87.61 |
| 9 | 59.99 |
| 10 | 99.35 |
| 11 | 18.19 |
| 12 | 107.54 |
| 13 | 101.67 |
| 14 | 100.45 |
| 15 | 100.40 |
| 16 | 55.30 |
| 17 | 54.79 |
| 18 | 96.62 |
| 19 | 95.81 |
| 20 | 74.62 |
| 21 | 71.67 |
| 22 | 85.33 |
| 23 | 95.77* |
| 24 | 98.10* |
| 25 | 90.49* |
| 26 | 91.67* |
| 27 | 89.22* |
| 28 | 62.56 |
| 29 | 99.74* |
| 30 | No data |
| 31 | 86.00* |
| 32 | 91.12* |
| 33 | 80.97 |
| 34 | 84.28 |
| 35 | 58.29 |
| 36 | No data |
| 37 | 76.29 |
| 38 | 62.33 |
| 39 | 63.01 |
| 40 | 72.38 |
| 41 | 72.67 |
| 42 | 77.52 |
| 43 | 74.34 |
| 44 | 78.37 |
| 45 | 81.93 |
| 46 | 93.48* |
| 47 | 74.75 |
| 48 | 79.84* |
| 49 | 85.56 |
| 50 | 77.81 |
| 51 | 88.84 |
| 52 | 69.48 |
| 53 | 70.48 |
| 54 | 77.50 |
| 55 | 44.37 |
| 56 | 46.29 |
| 57 | No data |
| 58 | 58.02 |
| 59 | 51.83 |
| 60 | 72.29 |
| 61 | 74.81 |
| 62 | 70.98 |
| 63 | 69.89 |
| 64 | 65.39 |
| 65 | 75.92 |
| 66 | 62.11 |
| 67 | 53.39 |
| 68 | 72.92 |
| 69 | 76.96 |
| 70 | 86.60 |
| 71 | 66.66 |
| 72 | 52.62 |
| 73 | 89.64 |
| 74 | 102.75 |
| 75 | 97.60 |
| 76 | 79.28 |
| 77 | 83.04 |
| 78 | 80.87 |
| 79 | No data |
| 80 | No data |
| 81 | 72.58 |
| 82 | 80.14 |
| 83 | No data |
| 84 | 79.57 |
| 85 | 66.79 |
| 86 | 91.06* |
| 87 | 96.55 |
| 88 | 101.86 |
| 89 | 85.32* |
| 90 | 24.59 |
| 91 | 83.09 |
| 92 | 3.56 |
| 93 | 79.62 |
| 94 | 77.52 |
| 95 | −0.48 |
| 96 | 62.18 |
| 97 | 75.87 |
| 98 | 59.40 |
| 99 | 81.99 |
| 100 | 74.23 |
| 101 | 8.69 |
| 102 | 73.52 |
| 103 | 79.47 |
| 104 | 76.25 |
| 105 | 79.20* |
| 106 | 40.76 |
| 107 | No data |
| 108 | −18.05 |
| 109 | 52.40 |
| 110 | 27.63 |
| 111 | −13.87 |
| 112 | 0.15 |
| 113 | 34.30 |
| 114 | No data |
| 115 | 28.78 |
| 116 | 82.02 |
| 117 | 73.36 |
| 118 | 80.57 |
| 119 | 32.04 |
| 120 | 73.88 |
| 121 | 49.63 |
| 122 | 57.89 |
| 123 | 66.40 |
| 124 | 31.26 |
| 125 | 17.53 |
| 126 | 35.73 |
| 127 | 26.47 |
| 128 | −16.23 |
| 129 | 74.86 |
| 130 | No data |
| 131 | No data |
| 132 | No data |
| 133 | No data |
| 134 | No data |
| 135 | No data |
| 136 | No data |
| 137 | No data |
| 138 | No data |
| 139 | 80.08 |
| 140 | 83.48 |
| 141 | 63.33 |
| 142 | 86.20 |
| 143 | 88.58 |
| 144 | 76.37 |
| 145 | 77.41 |
| 146 | 79.71 |
| 147 | 65.42 |
| 148 | 120.46 |
| 149 | 82.21 |
| 150 | 53.34 |
| 151 | 50.07 |
| 152 | 0.54 |
| 153 | No data |
| 154 | No data |
| 155 | No data |
| 156 | No data |
| 157 | No data |
| 158 | No data |
| 159 | No data |
| 160 | No data |
| 161 | 74.79 |
| 162 | No data |
| 163 | 54.95 |
| 164 | 46.95 |
| 165 | 56.92 |
| 166 | 37.43 |
| 167 | 31.15 |
| 168 | 66.36 |
| 169 | 34.23 |
| 170 | 45.98 |
| 171 | 3.62 |
| 172 | −32.84 |
| 173 | 34.72 |
| 174 | 52.08 |
| 175 | 80.01 |
| 176 | 88.95 |
| 177 | 68.87 |
| 178 | 83.02 |
| 179 | 93.94 |
| 180 | −56.89 |
| 181 | No data |
| 182 | No data |
| 183 | No data |
| 184 | No data |
| 185 | No data |
| 186 | No data |
| 187 | No data |
| 188 | No data |
| 189 | No data |
| 190 | No data |
| 191 | No data |
| 192 | No data |
| 193 | No data |
| 194 | No data |
| 195 | No data |
| 196 | No data |
| 197 | No data |
| 198 | No data |
| 199 | No data |
| 200 | No data |
| 201 | No data |
| 202 | No data |
| 203 | No data |
| 204 | No data |
| 205 | No data |
| 206 | No data |
| 207 | No data |
| 208 | No data |
| 209 | No data |
| 210 | No data |
| 211 | 76.62 |
| 212 | 14.52 |
| 213 | 55.80 |
| 214 | 101.05* |
| 215 | No data |
| 216 | 96.35* |
| 217 | 82.44 |
| 218 | 80.91 |
| 219 | 78.99 |
| 220 | 78.95 |
| 221 | 81.00 |
| 222 | 76.18 |
| 223 | 77.38 |
| 224 | 78.53 |
| 225 | 81.13 |
| 226 | 79.47 |
| 227 | 78.99 |
| 228 | 78.47 |
| 229 | 85.03* |
| 230 | 79.57 |
| 231 | 81.45 |
| 232 | 80.98* |
| 233 | 75.42 |
| 234 | 73.21 |
| 235 | 81.24 |
| 236 | 73.58 |
| 237 | 85.41 |
| 238 | 72.64 |
| 239 | 85.07 |
| 240 | 45.83 |
| 241 | 71.51 |
| 242 | 61.27 |
| 243 | 83.49 |
| 244 | 76.30 |
| 245 | 83.10 |
| 246 | 74.86 |
| 247 | 42.22 |
| 248 | 66.09 |
| 249 | 70.79 |
| 250 | 74.44 |
| 251 | 79.92 |
| 252 | 81.77 |
| 253 | 75.11 |
| 254 | 16.75 |
| 255 | 72.85 |
| 256 | 78.79 |
| 257 | 83.81 |
| 258 | 87.99 |
| 259 | 104.12 |
| 260 | 102.75* |
| 261 | 106.27 |
| 262 | 80.45 |
| 263 | 86.20 |
| 264 | 87.75 |
| 265 | 84.87 |
| 266 | 109.42 |
| 267 | 87.34 |
| 268 | 83.00 |
| 269 | 77.98 |
| 270 | 87.69 |
| 271 | 86.60 |
| 272 | 81.11 |
| 273 | 94.36* |
| 274 | 87.96 |
| 275 | 134.39 |
| 276 | 80.31* |
| 277 | 85.88 |
| 278 | 77.03 |
| 279 | 80.39 |
| 280 | 83.38 |
| 281 | 95.81 |
| 282 | 117.09 |
| 283 | 89.92 |
| 284 | 69.38 |
| 285 | 61.89 |
| 286 | 89.83 |
| 287 | 97.17* |
| 288 | 82.87* |
| 289 | 88.25 |
| 290 | 80.47* |
| 291 | 90.12 |
| 292 | 80.95 |
| 293 | 85.27 |
| 294 | 73.17 |
| 295 | 82.65 |
| 296 | 77.61 |
| 297 | 127.50 |
| 298 | 80.68 |
| 299 | 98.73 |
| 300 | 82.35 |
| 301 | 72.19 |
| 302 | 82.95 |
| 303 | 81.98 |
| 304 | 55.83 |
| 305 | 89.24 |
| 306 | 84.84* |
| 307 | 80.62 |
| 308 | 101.10* |
| 309 | 69.39 |
| 310 | 80.03 |
| 311 | 81.76 |
| 312 | 101.14* |
| 313 | 80.35 |
| 314 | 83.37 |
| 315 | 62.78 |
| 316 | 80.03 |
| 317 | 84.88* |
| 318 | 92.27 |
| 319 | 90.59* |
| 320 | 100.54* |
| 321 | 83.69 |
| 322 | 74.95 |
| 323 | 99.55* |
| 324 | 76.90 |
| 325 | 102.26 |
| 326 | 101.43 |
| 327 | 98.99 |
| 328 | 70.79 |
| 329 | 115.51 |
| 330 | 106.22 |
| 331 | 59.79 |
| 332 | 62.06 |
| 333 | 69.81 |
| 334 | 103.60 |
| 335 | 76.47 |
| 336 | 83.69 |
| 337 | 64.44 |
| 338 | 58.42 |
| 339 | 84.35 |
| 340 | 83.27 |
| 341 | 87.22 |
| 342 | 77.02 |
| 343 | −22.31 |
| 344 | 82.01 |
| 345 | 82.65 |
| 346 | 79.54 |
| 347 | 42.22 |
| 348 | 85.73* |
| 349 | 83.61 |
| 350 | 69.03 |
| 351 | 82.81 |
| 352 | 26.72 |
| 353 | 83.90 |
| 354 | 80.46 |
| 355 | 86.62* |
| 356 | 86.45 |
| 357 | 85.27 |
| 358 | 63.03 |
| 359 | 86.13* |
| 360 | 111.70 |
| 361 | 94.76 |
| 362 | 107.49 |
| 363 | 57.94 |
| 364 | 112.09 |
| 365 | 110.14 |
| 366 | 80.48 |
| 367 | 103.97 |
| 368 | 104.93 |
| 369 | 122.37 |
| 370 | 128.96 |
| 371 | 82.25 |
| 372 | 80.94 |
| 373 | 82.85 |
| 374 | 81.16 |
| 375 | 75.94 |
| 376 | 87.16 |
| 377 | 78.89 |
| 378 | 75.17 |
| 379 | No data |
| 380 | 37 |
| 381 | No data |
| 382 | No data |
| 383 | No data |
| 384 | No data |
| 385 | No data |
| 386 | No data |
| 387 | No data |
| 388 | 72 |
| 389 | 85 |
| 390 | No data |
| 391 | No data |
| 392 | 74 |
| 393 | 82 |
| 394 | 94 |
| 395 | No data |
| 396 | No data |
| 397 | No data |
| 398 | No data |
| 399 | No data |
| 400 | No data |
| 401 | No data |
| 402 | No data |
| 403 | 30 |
| 404 | 81 |
| 405 | 85 |
| 406 | 99 |
| 407 | 86 |
| 408 | 100 |
| 409 | 77 |
| 410 | 100 |
| 411 | No data |
| 412 | No data |
| 413 | No data |
| 414 | No data |
| 415 | No data |
| 416 | No data |
| 417 | No data |
| 418 | No data |
| 419 | No data |
| 420 | No data |
| 421 | No data |
| 422 | No data |
| 423 | No data |
| 424 | No data |
| 425 | No data |
| 426 | No data |
| 427 | No data |
| 428 | No data |
| 429 | No data |
| 430 | No data |
| 431 | No data |
| 432 | No data |
| 433 | No data |
| 434 | No data |
| 435 | 51 |
| 436 | 69 |
| 437 | 75 |
| 438 | 81 |
| 439 | 62 |
| 440 | 85 |
| 441 | 74 |
| 442 | No data |
| 443 | 85 |
| 444 | 7 |
| 445 | 34 |
| 446 | No data |
| 447 | 50 |
| 448 | 82 |
| 449 | No data |
| 450 | 86 |
| 451 | 70 |
| 452 | 76 |
| 453 | 92 |
| 454 | 92 |
| 455 | No data |
| 456 | 26 |
| 457 | 28 |
| 458 | No data |
| 459 | 100 |
| 460 | 100 |
| 461 | 100 |
| 462 | 85 |
| 463 | 67 |
| 464 | 36 |
| 465 | 59 |
| 466 | 78 |
| 467 | 76 |
| 468 | 80 |
| 469 | 64 |
| 470 | 49 |
| 471 | 84 |
| 472 | 100 |
| 473 | 62 |
| 474 | 100 |
| 475 | 93 |
| 476 | 100 |
| 477 | 100 |
| 478 | 100 |
| 470 | 3 |
| 480 | No data |
| 481 | No data |
| 482 | 99 |
| 483 | 66 |
| 484 | No data |
| 485 | 85 |
| 486 | No data |
| 487 | 84 |
| 488 | 92 |
| 489 | 97 |
| 490 | 100 |
| 491 | 87 |
| 492 | No data |
| 493 | 9 |
| 494 | No data |
| 495 | No data |
| 496 | 100 |
| 497 | 61 |
| 498 | 41 |
| 499 | 34 |
| 500 | 83 |
| 501 | No data |
| 502 | 10 |
| 503 | 147* |
| 504 | 83 |
| 505 | 19 |
| 506 | 33 |
| 507 | 74 |
| 508 | 0 |
| 509 | 100 |
| 510 | 97 |
| 511 | 100 |
| 512 | 100 |
| 513 | No data |
| 514 | No data |
| 515 | No data |
| 516 | 98* |
| 517 | 100 |
| 518 | 33 |
| 519 | No data |
| 520 | No data |
| 521 | No data |
| 522 | No data |
| 523 | No data |
| 524 | No data |
| 525 | No data |
| 526 | No data |
| 527 | No data |
| 528 | 100 |
| 529 | 100 |
| 530 | 100 |
| 531 | 100 |
| 532 | 100 |
| 533 | 100 |
| 534 | 49 |
| 535 | 100 |
| 536 | 100 |
| 537 | 100 |
| 538 | 100 |
| 539 | 100 |
| 540 | 100 |
| 541 | 97 |
| 542 | 85 |
| 543 | 68 |
| 544 | 100 |
| 545 | 100 |
| 546 | 100 |
| 547 | 71 |
| 548 | 95 |
| 549 | 94 |
| 550 | 100 |
| 551 | 100 |
| 552 | 100 |
| 553 | No data |
| 554 | No data |
| 555 | No data |
| 556 | No data |
| 557 | No data |
| 558 | No data |
| 559 | No data |
| 560 | No data |
| 561 | 99 |
| 562 | 100 |
| 563 | 100 |
| 564 | 92 |
| 565 | 100 |
| 566 | 52 |
| 567 | 100 |
| 568 | 100 |
| 569 | No data |
| 570 | 91 |
| 571 | 17 |
| 572 | 100 |
| 573 | 100 |
| 574 | 96 |
| 575 | 100 |
| 576 | 100 |
| 577 | No data |
| 578 | No data |
| 579 | No data |
| 580 | No data |
| 581 | No data |
| 582 | No data |
| 583 | No data |
| 584 | No data |
| 585 | 100 |
| 586 | 100 |
| 587 | 100 |
| 588 | 100 |
| 589 | 100 |
| 590 | 100 |
| 591 | 100 |
| 592 | 100 |
| 593 | No data |
| 594 | No data |
| 595 | No data |
| 596 | No data |
| 597 | No data |
| 598 | 82 |
| 599 | 94 |
| 600 | 100 |
| 601 | 100 |
| 602 | 100 |
| 603 | No data |
| 604 | 100 |
| 605 | No data |
| 606 | No data |
| 607 | No data |
| 608 | No data |
| 609 | No data |
| 610 | No data |
| 611 | No data |
| 612 | No data |
| 613 | No data |
| 614 | No data |
| 615 | 100 |
| 616 | 99 |
| 617 | 100 |
| 618 | 98 |
| 619 | 100 |
| 620 | 96 |
| 621 | 100 |
| 622 | 98 |
| 623 | 100 |
| 624 | 89 |
| 625 | No data |
| 626 | No data |
| 627 | No data |
| 628 | No data |
| 629 | No data |
| 630 | No data |
| 631 | No data |
| 632 | No data |
| 633 | No data |
| 634 | No data |
| 635 | 100 |
| 636 | 100 |
| 637 | 100 |
| 638 | 100 |
| 639 | 100 |
| 640 | 100 |
| 641 | 100 |
| 642 | 100 |
| 643 | 100 |
| 644 | 84 |
| 645 | No data |
| 646 | 100 |
| 647 | No data |
| 648 | 100 |
| 649 | No data |
| 650 | No data |
| 651 | No data |
| 652 | No data |
| 653 | 86 |
| 654 | No data |
| 655 | 89 |
| 656 | 78 |
| 657 | 100 |
| 658 | 96 |
| 659 | 100 |
| 660 | No data |
| 661 | No data |
| 662 | No data |
| 663 | No data |
| 664 | No data |
| 665 | No data |
| 666 | No data |
| 667 | 56 |
| 668 | 85 |
| 669 | 68 |
| 670 | 100 |
| 671 | 92 |
| 672 | 100 |
| 673 | 100 |
| 674 | No data |
| 675 | 100 |
| 676 | No data |
| 677 | 100 |
| 678 | 100 |
| 679 | No data |
| 680 | 100 |
| 681 | 100 |
| 682 | 100 |
| 683 | 100 |
| 684 | 100 |
| 685 | 100 |
| 686 | 100 |
| 687 | 100 |
| 688 | 99 |
| 689 | 99 |
| 690 | 100 |
| 691 | No data |
| 692 | 97 |
| 693 | 97 |
| 694 | 100 |
| 695 | 100 |
| 696 | No data |
| 697 | No data |
| 698 | No data |
| 699 | No data |
| 700 | No data |
| 701 | No data |
| 702 | No data |
| 703 | No data |
| 704 | No data |
| 705 | No data |
| 706 | No data |
| 707 | No data |
| 708 | No data |
| 709 | No data |
| 710 | No data |
| 711 | 100 |
| 712 | No data |
| 713 | No data |
| 714 | No data |
| 715 | No data |
| 716 | No data |
| 717 | No data |
| 718 | No data |
| 719 | No data |
| 720 | 100 |
| 721 | 100 |
| 722 | 93 |
| 723 | 94 |
| 724 | No data |
| 725 | No data |
| 726 | 100 |
| 727 | No data |
| 728 | 100 |
| 729 | 89 |
| 730 | No data |
| 731 | No data |
| 732 | 100 |
| 733 | 100 |
| 734 | No data |
| 735 | No data |
| 736 | 100 |
| 737 | 97 |
| 738 | No data |
| 739 | No data |
| 740 | No data |
| 741 | No data |
| 742 | 100 |
| 743 | 94 |
| 744 | 100 |
| 745 | 100 |
| 746 | No data |
| 747 | No data |
| 748 | No data |
| 749 | No data |
| 750 | No data |
| 751 | No data |
| 752 | No data |
| 753 | No data |
| 754 | 100 |
| 755 | 100 |
| 756 | 56 |
| 757 | 19 |
| 758 | 98 |
| 759 | 100 |
| 760 | 100 |
| 761 | 100 |
| 762 | No data |
| 763 | No data |
| 764 | No data |
| 765 | No data |
| 766 | No data |
| 767 | No data |
| 768 | No data |
| 769 | No data |
| 770 | 100 |
| 771 | 100 |
| 772 | 81 |
| 773 | 55 |
| 774 | 100 |
| 775 | 100 |
| 776 | 100 |
| 777 | 100 |
| 778 | No data |
| 779 | No data |
| 780 | 100 |
| 781 | 99 |
| 782 | No data |
| 783 | No data |
| 784 | 70 |
| 785 | 100 |
| 786 | No data |
| 787 | 97 |
| 788 | No data |
| 789 | 99 |
| 790 | No data |
| 791 | 97 |
| 792 | No data |
| 793 | 100 |
| 794 | No data |
| 795 | 100 |
| 796 | No data |
| 797 | 100 |
| 798 | No data |
| 799 | 100 |
| 800 | 99 |
| 801 | No data |
| 802 | 100 |
| 803 | 100 |
| 804 | No data |
| 805 | 100 |
| 806 | No data |
| 807 | 100 |
| 808 | 100 |
| 809 | 100 |
| 810 | 100 |
| 811 | 100 |
| 812 | 100 |
| 813 | 100 |
| 814 | 100 |
| 815 | 100 |
| 816 | 100 |
| 817 | 100 |
| 818 | 100 |
| 819 | 100 |
| 820 | 100 |
| 821 | 100 |
| 822 | 100 |
| 823 | 100 |
| 824 | 100 |
| 825 | 100 |
| 826 | 96 |
| 827 | 100 |
| 828 | 100 |
| 829 | 100 |
| 830 | 100 |
| 831 | 89 |
| 832 | 100 |
| 833 | 100 |
| 834 | 98 |
| 835 | 95 |
| 836 | 94 |
| 837 | 94 |
| 838 | 99 |
| 839 | 100 |
| 840 | 99 |
| 841 | No data |
| 842 | 97 |
| 843 | No data |
| 844 | 100 |
| 845 | 92 |
| 846 | 97 |
| 847 | 97 |
| 848 | 100 |
| 849 | No data |
| 850 | No data |
| 851 | 98 |
| 852 | 100 |
| 853 | No data |
| 854 | 100 |
| 855 | 94 |
| 856 | 100 |
| 857 | 100 |
| 858 | No data |
| 859 | 100 |
| 860 | No data |
| 861 | 100 |
| 862 | No data |
| 863 | 100 |
| 864 | No data |
| 865 | No data |
| 866 | No data |
| 867 | No data |
| 868 | 6 |
| 869 | 93 |
| 870 | 100 |
| 871 | 100 |
| 872 | 100 |
| 873 | 100 |
| 874 | 100 |
| 875 | 98 |
| 876 | 0 |
| 877 | 100 |
| 878 | 88 |
| 879 | 91 |
| 880 | 91 |
| 881 | 97 |
| 882 | 100 |
| 883 | 99 |
| 884 | 100 |
| 885 | 100 |
| 886 | 0.008* |
| 887 | 100 |
| 888 | No data |
| 889 | No data |
| 890 | 91 |
| 891 | 41 |
| 892 | 100 |
| 893 | 99 |
| 894 | No data |
| 895 | No data |
| 896 | No data |
| 897 | 100 |
| 898 | 100 |
| 899 | 97 |
| 900 | 100 |
| 901 | 97 |
| 902 | 97 |
| 903 | No data |
| 904 | No data |
| 905 | No data |
| 906 | No data |
| 907 | No data |
| 908 | No data |
| 909 | 100 |
| 910 | 3.4* |
| 911 | 58 |
| 912 | 99 |
| 913 | 100 |
| 914 | 100 |
| 915 | 100 |
| 916 | 99 |
| 917 | 100 |
| 918 | 100 |
| 919 | 99 |
| 920 | 100 |
| 921 | 99 |
| 922 | 100 |
| 923 | 100 |
| 924 | 97 |
| 925 | 88 |
| 926 | 100 |
| 927 | 98 |
| 928 | 85 |
| 929 | 82 |
| 930 | 72 |
| 931 | 100 |
| 932 | 100 |
| 933 | 100 |
| 934 | 100 |
| 935 | 100 |
| 936 | 100 |
| 937 | 17 |
| 938 | 91 |
| 939 | No data |
| 940 | 76 |
| 941 | 54 |
| 942 | 100 |
| 943 | No data |
| 944 | No data |
| 945 | No data |
| 946 | No data |
| 947 | No data |
| 948 | No data |
| 949 | No data |
| 950 | No data |
| 951 | No data |
| 952 | No data |
| 953 | No data |
| 954 | No data |
| 955 | No data |
| 956 | 100 |
| 957 | 100 |
| 958 | 93 |
| 959 | 93 |
| 960 | 100 |
| 961 | 100 |
| 962 | 98 |
| 963 | 100 |
| 964 | 40 |
| 965 | 100 |
| 966 | 100 |
| 967 | 100 |
| 968 | 94 |
| 969 | 100 |
| 970 | 100 |
| 971 | 100 |
| 972 | 100 |
| 973 | No data |
| 974 | 89 |
| 975 | 100 |
| 976 | No data |
| 977 | 74 |
| 978 | 82 |
| 979 | No data |
| 980 | No data |
| 981 | No data |
| 982 | 100 |
| 983 | No data |
| 984 | No data |
| 985 | No data |
| 986 | No data |
| 987 | 30 |
| 988 | 69 |
| 989 | 100 |
| 990 | 100 |
| 991 | No data |
| 992 | No data |
| 993 | 100 |
| 994 | No data |
| 995 | 89 |
| 996 | No data |
| 997 | 100 |
| 998 | 100 |
| 999 | No data |
| 1000 | 64 |
| 1001 | No data |
| 1002 | 100 |
| 1003 | No data |
| 1004 | No data |
| 1005 | No data |
| 1006 | No data |
| 1007 | No data |
| 1008 | 83 |
| 1009 | 92 |
| 1010 | 81 |
| 1011 | No data |
| 1012 | No data |
| 1013 | No data |
| 1014 | No data |
| 1015 | No data |
| 1016 | 94 |
| 1017 | 90 |
| 1018 | No data |
| 1019 | 100 |
| 1020 | No data |
| 1021 | No data |
| 1022 | No data |
| 1023 | No data |
| 1024 | 100 |
| 1025 | 98 |
| 1026 | 98 |
| 1027 | 94 |
| 1028 | No data |
| 1029 | No data |
| 1030 | 100 |
| 1031 | No data |
| 1032 | No data |
| 1033 | 20 |
| 1034 | 96 |
| 1035 | 51 |
| 1036 | 100 |
| 1037 | No data |
| 1038 | No data |
| 1039 | 90 |
| 1040 | 86 |
| 1041 | 100 |
| 1042 | 49 |
| 1043 | No data |
| 1044 | No data |
| 1045 | No data |
| 1046 | No data |
| 1047 | No data |
| 1048 | 100 |
| 1049 | 99 |
| 1050 | No data |
| 1051 | 0 |
| 1052 | 0 |
| 1053 | 0 |
| 1054 | 0 |
| 1055 | No data |
| 1056 | 85 |
| 1057 | 83 |
| 1058 | 87 |
| 1059 | 85 |
| 1060 | 87 |
| 1061 | 92 |
| 1062 | 91 |
| 1063 | 94 |
| 1064 | 100 |
| 1065 | 68 |
| 1066 | 100 |
| 1067 | 98 |
| 1068 | 100 |
| 1069 | 100 |
| 1070 | 93 |
| 1071 | 100 |
| 1072 | 95 |
| 1073 | 99 |
| 1074 | 100 |
| 1075 | 97 |
| 1076 | 99 |
| 1077 | 99 |
| 1078 | 100 |
| 1079 | 100 |
| 1080 | 100 |
| 1081 | 100 |
| 1082 | 100 |
| 1083 | 99 |
| 1084 | 100 |
| 1085 | 100 |
| 1086 | 92 |
| 1087 | 100 |
| 1088 | 100 |
| 1089 | 100 |
| 1090 | 100 |
| 1091 | 100 |
| 1092 | 100 |
| 1093 | 100 |
| 1094 | 99 |
| 1095 | 100 |
| 1096 | 100 |
| 1097 | 100 |
| 1098 | 100 |
| 1099 | 100 |
| 1100 | 75 |
| 1101 | 82 |
| 1102 | 96 |
| 1103 | 76 |
| 1104 | 0.014* |
| 1105 | 82 |
| 1106 | 21* |
| 1107 | <0.5* |
| 1108 | 18 |
| 1109 | 28 |
| 1110 | 99 |
| 1111 | 88 |
| 1112 | 71 |
| 1113 | 72 |
| 1114 | 100 |
| 1115 | 93 |
| 1116 | 97 |
| 1117 | No data |
| 1118 | No data |
| 1119 | No data |
| 1120 | 79 |
| 1121 | 99 |
| 1122 | 100 |
| 1123 | 100 |
| 1124 | 42 |
| 1125 | 51 |
| 1126 | 100 |
| 1127 | 100 |
| 1128 | 67 |
| 1129 | 83 |
| 1130 | 99 |
| 1131 | 100 |
| 1132 | 100 |
| 1133 | 100 |
| 1134 | 100 |
| 1135 | 100 |
| 1136 | 99 |
| 1137 | 100 |
| 1138 | 94 |
| 1139 | 96 |
| 1140 | 100 |
| 1141 | 100 |
| 1142 | 100 |
| 1143 | 100 |
| 1144 | 100 |
| 1145 | 100 |
| 1146 | 100 |
| 1147 | 100 |
| 1148 | 24 |
| 1149 | 100 |
| 1150 | 100 |
| 1151 | 100 |
| 1152 | 100 |
| 1153 | 100 |
| 1154 | No data |
| 1155 | No data |
| 1156 | 88 |
| 1157 | No data |
| 1158 | No data |
| 1159 | 100 |
| 1160 | 100 |
| 1161 | 81 |
| 1162 | 100 |
| 1163 | 100 |
| 1164 | 100 |
| 1165 | No data |
| 1166 | 100 |
| 1167 | 100 |
| 1168 | 100 |
| 1169 | 100 |
| 1170 | 100 |
| 1171 | No data |
| 1172 | 100 |
| 1173 | 100 |
| 1174 | 100 |
| 1175 | 99 |
| 1176 | 100 |

Note:
Examples which are marked with “*” are an average of two or more data points.

### Bacterial Susceptibility Testing Methods

Compounds may be tested for antimicrobial activity by susceptibility testing in liquid media. Compounds may be dissolved in dimethylsulfoxide and tested in 10 doubling dilutions in the susceptibility assays. The organisms used in the assay may be grown overnight on suitable agar media and then suspended in a liquid medium appropriate for the growth of the organism. The suspension can be a 0.5 McFarland and a further 1 in 10 dilution can be made into the same liquid medium to prepare the final organism suspension in 100 μL. Plates can be incubated under appropriate conditions at 37° C. for 24 hrs prior to reading. The Minimum Inhibitory Concentration (MIC) may be determined as the lowest drug concentration able to reduce growth by 80% or more.

In an assay comparable to the above, compounds of the invention had MICs as shown in the table below:

| | Spn 548(gram+) | Spy 838 (gram+) | Mca 445 (gram−) |
|---|---|---|---|
| Example | (μM) | (μM) | (μM) |
| 46 | 3.13 | 8.84 | 3.13 |
| 176 | 6.25 | 12.5 | 0.78 |
| 211 | 25.00 | 25.00 | 12.5 |

According to a further feature of the invention there is provided a compound of the formula (I), or a pharmaceutically-acceptable salt thereof, for use in a method of treatment of the human or animal body by therapy.

In one embodiment, the invention provides a method of treating a bacterial infection in an animal, such as a human, comprising administering to the animal or human an effective amount of a compound of any one of formulas (I), or a pharmaceutically acceptable salt thereof.

We have found that compounds of the present invention inhibit bacterial DNA gyrase and/or topoisomerase IV and are therefore of interest for their antibacterial effects. In one aspect of the invention the compounds of the invention inhibit bacterial DNA gyrase and are therefore of interest for their antibacterial effects. In one aspect of the invention, the compounds of the invention inhibit topoisomerase IV and are therefore of interest for their antibacterial effects. In one aspect of the invention, the compounds of the invention inhibit both DNA gyrase and topoisomerase IV and are therefore of interest for their antibacterial effects. Thus, the compounds of the invention are useful in treating or preventing bacterial infections.

In one aspect of the invention an “infection” or “bacterial infection” refers to an infection caused by Acinetobacter baumanii. In one aspect of the invention an “infection” or “bacterial infection” refers to an infection caused by Acinetobacter haemolyticus. In one aspect of the invention an “infection” or “bacterial infection” refers to an infection caused by Acinetobacter junii. In one aspect of the invention an “infection” or “bacterial infection” refers to an infection caused by Acinetobacter johnsonii. In one aspect of the invention an “infection” or “bacterial infection” refers to an infection caused by Acinetobacter lwoffi. In one aspect of the invention an “infection” or “bacterial infection” refers to an infection caused by Bacteroides bivius. In one aspect of the invention an “infection” or “bacterial infection” refers to an infection caused by Bacteroides fragilis. In one aspect of the invention an “infection” or “bacterial infection” refers to an infection caused by Burkholderia cepacia. In one aspect of the invention an “infection” or “bacterial infection” refers to an infection caused by Campylobacter jejuni. In one aspect of the invention an “infection” or “bacterial infection” refers to an infection caused by Chlamydia pneumoniae. In one aspect of the invention an “infection” or “bacterial infection” refers to an infection caused by Chlamydia urealyticus. In one aspect of the invention an “infection” or “bacterial infection” refers to an infection caused by Chlamydophila pneumoniae. In one aspect of the invention an “infection” or “bacterial infection” refers to an infection caused by Clostridium difficile. In one aspect of the invention an “infection” or “bacterial infection” refers to an infection caused by Enterobacter aerogenes. In one aspect of the invention an “infection” or “bacterial infection” refers to an infection caused by Enterobacter cloacae. In one aspect of the invention an “infection” or “bacterial infection” refers to an infection caused by Enterococcus faecalis. In one aspect of the invention an “infection” or “bacterial infection” refers to an infection caused by Enterococcus faecium. In one aspect of the invention an “infection” or “bacterial infection” refers to an infection caused by Escherichia coli. In one aspect of the invention an “infection” or “bacterial infection” refers to an infection caused by Gardnerella vaginalis. In one aspect of the invention an “infection” or “bacterial infection” refers to an infection caused by Haemophilus parainfluenzae. In one aspect of the invention an “infection” or “bacterial infection” refers to an infection caused by Haemophilus influenzae. In one aspect of the invention an “infection” or “bacterial infection” refers to an infection caused by Helicobacter pylori. In one aspect of the invention an “infection” or “bacterial infection” refers to an infection caused by Klebsiella pneumoniae. In one aspect of the invention an “infection” or “bacterial infection” refers to an infection caused by Legionella pneumophila. In one aspect of the invention an “infection” or “bacterial infection” refers to an infection caused by Methicillin-resistant Staphylococcus aureus. In one aspect of the invention an “infection” or “bacterial infection” refers to an infection caused by Methicillin-susceptible Staphylococcus aureus. In one aspect of the invention an “infection” or “bacterial infection” refers to an infection caused by Moraxella catarrhalis. In one aspect of the invention an “infection” or “bacterial infection” refers to an infection caused by Morganella morganii. In one aspect of the invention an “infection” or “bacterial infection” refers to an infection caused by Mycoplasma pneumoniae. In one aspect of the invention an “infection” or “bacterial infection” refers to an infection caused by Neisseria gonorrhoeae. In one aspect of the invention an “infection” or “bacterial infection” refers to an infection caused by Penicillin-resistant Streptococcus pneumoniae. In one aspect of the invention an “infection” or “bacterial infection” refers to an infection caused by Penicillin-susceptible Streptococcus pneumoniae.In one aspect of the invention an “infection” or “bacterial infection” refers to an infection caused by Peptostreptococcus magnus. In one aspect of the invention an “infection” or “bacterial infection” refers to an infection caused by Peptostreptococcus micros. In one aspect of the invention an “infection” or “bacterial infection” refers to an infection caused by Peptostreptococcus anaerobius. In one aspect of the invention an “infection” or “bacterial infection” refers to an infection caused by Peptostreptococcus asaccharolyticus. In one aspect of the invention an “infection” or “bacterial infection” refers to an infection caused by Peptostreptococcus prevotii. In one aspect of the invention an “infection” or “bacterial infection” refers to an infection caused by Peptostreptococcus tetradius. In one aspect of the invention an “infection” or “bacterial infection” refers to an infection caused by Peptostreptococcus vaginalis. In one aspect of the invention an “infection” or “bacterial infection” refers to an infection caused by Proteus mirabilis. In one aspect of the invention an “infection” or “bacterial infection” refers to an infection caused by Pseudomonas aeruginosa. In one aspect of the invention an “infection” or “bacterial infection” refers to an infection caused by Quinolone-Resistant Staphylococcus aureus. In one aspect of the invention an “infection” or “bacterial infection” refers to an infection caused by Quinolone-Resistant Staphylococcus epidermis. In one aspect of the invention an “infection” or “bacterial infection” refers to an infection caused by Salmonella typhi. In one aspect of the invention an “infection” or “bacterial infection” refers to an infection caused by Salmonella paratyphi. In one aspect of the invention an “infection” or “bacterial infection” refers to an infection caused by Salmonella enteritidis. In one aspect of the invention an “infection” or “bacterial infection” refers to an infection caused by Salmonella typhimurium. In one aspect of the invention an “infection” or “bacterial infection” refers to an infection caused by Serratia marcescens. In one aspect of the invention an “infection” or “bacterial infection” refers to an infection caused by Staphylococcus aureus. In one aspect of the invention an “infection” or “bacterial infection” refers to an infection caused by Staphylococcus epidermidis. In one aspect of the invention an “infection” or “bacterial infection” refers to an infection caused by Staphylococcus saprophyticus. In one aspect of the invention an “infection” or “bacterial infection” refers to an infection caused by Streptoccocus agalactiae. In one aspect of the invention an “infection” or “bacterial infection” refers to an infection caused by Streptococcus pneumoniae. In one aspect of the invention an “infection” or “bacterial infection” refers to an infection caused by Streptococcus pyogenes. In one aspect of the invention an “infection” or “bacterial infection” refers to an infection caused by Stenotrophomonas maltophilia. In one aspect of the invention an “infection” or “bacterial infection” refers to an infection caused by Ureaplasma urealyticum. In one aspect of the invention an “infection” or “bacterial infection” refers to an infection caused by Vancomycin-Resistant Enterococcus faecium. In one aspect of the invention an “infection” or “bacterial infection” refers to an infection caused by Vancomycin-Resistant Enterococcus faecalis. In one aspect of the invention an “infection” or “bacterial infection” refers to an infection caused by Vancomycin-Resistant Staphylococcus aureus. In one aspect of the invention an “infection” or “bacterial infection” refers to an infection caused by Vancomycin-Resistant Staphylococcus epidermis. In one aspect of the invention an “infection” or “bacterial infection” refers to an infection caused by Mycobacterium tuberculosis. In one aspect of the invention an “infection” or “bacterial infection” refers to an infection caused by Clostridium perfringens. In one aspect of the invention an “infection” or “bacterial infection” refers to an infection caused by Klebsiella oxytoca. In one aspect of the invention an “infection” or “bacterial infection” refers to an infection caused by Neisseria miningitidis. In one aspect of the invention an “infection” or “bacterial infection” refers to an infection caused by Fusobacterium spp. In one aspect of the invention an “infection” or “bacterial infection” refers to an infection caused by Peptococcus spp. In one aspect of the invention an “infection” or “bacterial infection” refers to an infection caused by Proteus vulgaris. In one aspect of the invention an “infection” or “bacterial infection” refers to an infection caused by Coagulase-negative Staphylococcus (including Staphylococcus lugdunensis, Staphylococcus capitis, Staphylococcus hominis, and Staphylococcus saprophyticus).

In one aspect of the invention an “infection” or “bacterial infection” refers to an infection caused by Acinetobacter spp. In one aspect of the invention an “infection” or “bacterial infection” refers to an infection caused by Bacteroides spp. In one aspect of the invention an “infection” or “bacterial infection” refers to an infection caused by Burkholderia spp. In one aspect of the invention an “infection” or “bacterial infection” refers to an infection caused by Campylobacter spp. In one aspect of the invention an “infection” or “bacterial infection” refers to an infection caused by Chlamydia spp. In one aspect of the invention an “infection” or “bacterial infection” refers to an infection caused by Chlamydophila spp. In one aspect of the invention an “infection” or “bacterial infection” refers to an infection caused by Clostridium spp. In one aspect of the invention an “infection” or “bacterial infection” refers to an infection caused by Enterobacter spp. In one aspect of the invention an “infection” or “bacterial infection” refers to an infection caused by Enterococcus spp. In one aspect of the invention an “infection” or “bacterial infection” refers to an infection caused by Escherichia spp. In one aspect of the invention an “infection” or “bacterial infection” refers to an infection caused by Gardnerella spp. In one aspect of the invention an “infection” or “bacterial infection” refers to an infection caused by Haemophilus spp. In one aspect of the invention an “infection” or “bacterial infection” refers to an infection caused by Helicobacter spp. In one aspect of the invention an “infection” or “bacterial infection” refers to an infection caused by Klebsiella spp. In one aspect of the invention an “infection” or “bacterial infection” refers to an infection caused by Legionella spp. In one aspect of the invention an “infection” or “bacterial infection” refers to an infection caused by Moraxella spp. In one aspect of the invention an “infection” or “bacterial infection” refers to an infection caused by Morganella spp. In one aspect of the invention an “infection” or “bacterial infection” refers to an infection caused by Mycoplasma spp. In one aspect of the invention an “infection” or “bacterial infection” refers to an infection caused by Neisseria spp. In one aspect of the invention an “infection” or “bacterial infection” refers to an infection caused by Peptostreptococcus spp. In one aspect of the invention an “infection” or “bacterial infection” refers to an infection caused by Proteus spp. In one aspect of the invention an “infection” or “bacterial infection” refers to an infection caused by Pseudomonas spp. In one aspect of the invention an “infection” or “bacterial infection” refers to an infection caused by Salmonella spp. In one aspect of the invention an “infection” or “bacterial infection” refers to an infection caused by Serratia spp. In one aspect of the invention an “infection” or “bacterial infection” refers to an infection caused by Staphylococcus spp. In one aspect of the invention an “infection” or “bacterial infection” refers to an infection caused by Streptoccocus spp. In one aspect of the invention an “infection” or “bacterial infection” refers to an infection caused by Stenotrophomonas spp. In one aspect of the invention an “infection” or “bacterial infection” refers to an infection caused by Ureaplasma spp. In one aspect of the invention an “infection” or “bacterial infection” refers to an infection caused by aerobes. In one aspect of the invention an “infection” or “bacterial infection” refers to an infection caused by obligate anaerobes. In one aspect of the invention an “infection” or “bacterial infection” refers to an infection caused by facultative anaerobes. In one aspect of the invention an “infection” or “bacterial infection” refers to an infection caused by gram-positive bacteria. In one aspect of the invention an “infection” or “bacterial infection” refers to an infection caused by gram-negative bacteria. In one aspect of the invention an “infection” or “bacterial infection” refers to an infection caused by gram-variable bacteria. In one aspect of the invention an “infection” or “bacterial infection” refers to an infection caused by atypical respiratory pathogens. In one aspect of the invention an “infection” or “bacterial infection” refers to an infection caused by Enterics. In one aspect of the invention an “infection” or “bacterial infection” refers to an infection caused by Shigella spp. In one aspect of the invention an “infection” or “bacterial infection” refers to an infection caused by Citrobacter.

In one aspect of the invention “infection” or “bacterial infection” refers to a gynecological infection. In one aspect of the invention “infection” or “bacterial infection” refers to a respiratory tract infection (RTI). In one aspect of the invention “infection” or “bacterial infection” refers to a sexually transmitted disease. In one aspect of the invention “infection” or “bacterial infection” refers to a urinary tract infection. In one aspect of the invention “infection” or “bacterial infection” refers to acute exacerbation of chronic bronchitis (ACEB). In one aspect of the invention “infection” or “bacterial infection” refers to acute otitis media. In one aspect of the invention “infection” or “bacterial infection” refers to acute sinusitis. In one aspect of the invention “infection” or “bacterial infection” refers to an infection caused by drug resistant bacteria. In one aspect of the invention “infection” or “bacterial infection” refers to catheter-related sepsis. In one aspect of the invention “infection” or “bacterial infection” refers to chancroid. In one aspect of the invention “infection” or “bacterial infection” refers to chlamydia. In one aspect of the invention “infection” or “bacterial infection” refers to community-acquired pneumonia (CAP). In one aspect of the invention “infection” or “bacterial infection” refers to complicated skin and skin structure infection. In one aspect of the invention “infection” or “bacterial infection” refers to uncomplicated skin and skin structure infection. In one aspect of the invention “infection” or “bacterial infection” refers to endocarditis. In one aspect of the invention “infection” or “bacterial infection” refers to febrile neutropenia. In one aspect of the invention “infection” or “bacterial infection” refers to gonococcal cervicitis. In one aspect of the invention “infection” or “bacterial infection” refers to gonococcal urethritis. In one aspect of the invention “infection” or “bacterial infection” refers to hospital-acquired pneumonia (HAP). In one aspect of the invention “infection” or “bacterial infection” refers to osteomyelitis. In one aspect of the invention “infection” or “bacterial infection” refers to sepsis. In one aspect of the invention “infection” or “bacterial infection” refers to syphilis. In one aspect of the invention “infection” or “bacterial infection” refers to ventilator-associated pneumonia. In one aspect of the invention “infection” or “bacterial infection” refers to intraabdominal infections. In one aspect of the invention “infection” or “bacterial infection” refers to gonorrhoeae. In one aspect of the invention “infection” or “bacterial infection” refers to meningitis. In one aspect of the invention “infection” or “bacterial infection” refers to tetanus. In one aspect of the invention “infection” or “bacterial infection” refers to tuberculosis.

In one embodiment, it is expected that the compounds of the present invention will be useful in treating bacterial infections including, but not limited to community-acquired pneumoniae, hospital-acquired pneumoniae, skin & skin structure infections, acute exacerbation of chronic bronchitis, acute sinusitis, acute otitis media, catheter-related sepsis, febrile neutropenia, osteomyelitis, endocarditis, urinary tract infections and infections caused by drug resistant bacteria such as Penicillin-resistant Streptococcus pneumoniae, methicillin-resistant Staphylococcus aureus, methicillin-resistant Staphylococcus epidermidis and Vancomycin-Resistant Enterococci.

According to a further feature of the present invention there is provided a method for producing an antibacterial effect in a warm blooded animal, such as man, in need of such treatment, which comprises administering to said animal an effective amount of a compound of the present invention, or a pharmaceutically-acceptable salt thereof.

According to a further feature of the invention there is provided a method for inhibition of bacterial DNA gyrase and/or topoisomerase IV in a warm-blooded animal, such as a human being, in need of such treatment which comprises administering to said animal an effective amount of a compound of formula (I), or a pharmaceutically acceptable salt thereof as defined hereinbefore.

According to a further feature of the invention there is provided a method of treating a bacterial infection in a warm-blooded animal, such as a human being, in need of such treatment which comprises administering to said animal an effective amount of a compound of formula (I), or a pharmaceutically acceptable salt thereof as defined hereinbefore.

According to a further feature of the invention there is provided a method of treating a bacterial infection selected from community-acquired pneumoniae, hospital-acquired pneumoniae, skin & skin structure infections, acute exacerbation of chronic bronchitis, acute sinusitis, acute otitis media, catheter-related sepsis, febrile neutropenia, osteomyelitis, endocarditis, urinary tract infections and infections caused by drug resistant bacteria such as Penicillin-resistant Streptococcus pneumoniae, methicillin-resistant Staphylococcus aureus, methicillin-resistant Staphylococcus epidermidis and Vancomycin-Resistant Enterococciin a warm-blooded animal, such as a human being, in need of such treatment which comprises administering to said animal an effective amount of a compound of formula (I), or a pharmaceutically acceptable salt thereof as defined hereinbefore.

A further feature of the present invention is a compound of formula (I), and pharmaceutically acceptable salts thereof for use as a medicament. Suitably the medicament is an antibacterial agent.

According to a further aspect of the invention there is provided the use of a compound of formula (I), or a pharmaceutically acceptable salt thereof in the manufacture of a medicament for use in the production of an anti-bacterial effect in a warm-blooded animal such as a human being.

According to a further aspect of the invention there is provided the use of a compound of formula (I), or a pharmaceutically acceptable salt thereof, in the manufacture of a medicament for use in inhibition of bacterial DNA gyrase and/or topoisomerase IV in a warm-blooded animal such as a human being.

Thus according to a further aspect of the invention there is provided the use of a compound of formula (I), or a pharmaceutically acceptable salt thereof, in the manufacture of a medicament for use in the treatment of a bacterial infection in a warm-blooded animal such as a human being.

Thus according to a further aspect of the invention there is provided the use of a compound of formula (I), or a pharmaceutically acceptable salt thereof, in the manufacture of a medicament for use in the treatment of a bacterial infection selected from community-acquired pneumoniae, hospital-acquired pneumoniae, skin & skin structure infections, acute exacerbation of chronic bronchitis, acute sinusitis, acute otitis media, catheter-related sepsis, febrile neutropenia, osteomyelitis, endocarditis, urinary tract infections and infections caused by drug resistant bacteria such as Penicillin-resistant Streptococcus pneumoniae, methicillin-resistant Staphylococcus aureus, methicillin-resistant Staphylococcus epidermidis and Vancomycin-Resistant Enterococci in a warm-blooded animal such as a human being.

According to a further aspect of the invention there is provided a compound of formula (I), or a pharmaceutically acceptable salt thereof, for use in the production of an anti-bacterial effect in a warm-blooded animal such as a human being.

According to a further aspect of the invention there is provided a compound of formula (I), or a pharmaceutically acceptable salt thereof, for use in inhibition of bacterial DNA gyrase and/or topoisomerase IV in a warm-blooded animal such as a human being.

Thus according to a further aspect of the invention there is provided a compound of formula (I), or a pharmaceutically acceptable salt thereof, for use in the treatment of a bacterial infection in a warm-blooded animal such as a human being.

Thus according to a further aspect of the invention there is provided a compound of formula (I), or a pharmaceutically acceptable salt thereof, for use in the treatment of a bacterial infection selected from community-acquired pneumoniae, hospital-acquired pneumoniae, skin & skin structure infections, acute exacerbation of chronic bronchitis, acute sinusitis, acute otitis media, catheter-related sepsis, febrile neutropenia, osteomyelitis, endocarditis, urinary tract infections and infections caused by drug resistant bacteria such as Penicillin-resistant Streptococcus pneumoniae, methicillin-resistant Staphylococcus aureus, methicillin-resistant Staphylococcus epidermidis and Vancomycin-Resistant Enterococci in a warm-blooded animal such as a human being.

In order to use a compound of the formula (I), or a pharmaceutically-acceptable salt thereof, (hereinafter in this section relating to pharmaceutical composition “a compound of this invention”) for the therapeutic (including prophylactic) treatment of mammals including humans, in particular in treating infection, it is normally formulated in accordance with standard pharmaceutical practice as a pharmaceutical composition.

Therefore in another aspect the present invention provides a pharmaceutical composition which comprises a compound of the formula (I), or a pharmaceutically-acceptable salt thereof, and a pharmaceutically-acceptable diluent or carrier.

According to a further aspect of the invention there is provided a pharmaceutical composition which comprises a compound of formula (I), as defined hereinbefore or a pharmaceutically acceptable salt thereof, in association with a pharmaceutically acceptable excipient or carrier for use in producing an anti-bacterial effect in an warm-blooded animal, such as a human being.

According to a further aspect of the invention there is provided a pharmaceutical composition which comprises a compound of formula (I), as defined hereinbefore or a pharmaceutically acceptable salt thereof, in association with a pharmaceutically acceptable excipient or carrier for use in inhibition of bacterial DNA gyrase and/or topoisomerase IV in an warm-blooded animal, such as a human being.

According to a further aspect of the invention there is provided a pharmaceutical composition which comprises a compound of formula (I), as defined hereinbefore or a pharmaceutically acceptable salt thereof, in association with a pharmaceutically acceptable excipient or carrier for use in the treatment of a bacterial infection in an warm-blooded animal, such as a human being.

According to a further aspect of the invention there is provided a pharmaceutical composition which comprises a compound of formula (I), as defined hereinbefore or a pharmaceutically acceptable salt thereof, in association with a pharmaceutically acceptable excipient or carrier for use in the treatment of a bacterial infection selected from community-acquired pneumoniae, hospital-acquired pneumoniae, skin & skin structure infections, acute exacerbation of chronic bronchitis, acute sinusitis, acute otitis media, catheter-related sepsis, febrile neutropenia, osteomyelitis, endocarditis, urinary tract infections and infections caused by drug resistant bacteria such as Penicillin-resistant Streptococcus pneumoniae, methicillin-resistant Staphylococcus aureus, methicillin-resistant Staphylococcus epidermidis and Vancomycin-Resistant Enterococci in an warm-blooded animal, such as a human being.

The compositions of the invention may be in a form suitable for oral use (for example as tablets, lozenges, hard or soft capsules, aqueous or oily suspensions, emulsions, dispersible powders or granules, syrups or elixirs), for topical use (for example as creams, ointments, gels, or aqueous or oily solutions or suspensions), for administration by inhalation (for example as a finely divided powder or a liquid aerosol), for administration by insufflation (for example as a finely divided powder) or for parenteral administration (for example as a sterile aqueous or oily solution for intravenous, subcutaneous, intramuscular or intramuscular dosing or as a suppository for rectal dosing).

The compositions of the invention may be obtained by conventional procedures using conventional pharmaceutical excipients, well known in the art. Thus, compositions intended for oral use may contain, for example, one or more coloring, sweetening, flavoring and/or preservative agents.

Suitable pharmaceutically acceptable excipients for a tablet formulation include, for example, inert diluents such as lactose, sodium carbonate, calcium phosphate or calcium carbonate, granulating and disintegrating agents such as corn starch or algenic acid; binding agents such as starch; lubricating agents such as magnesium stearate, stearic acid or talc; preservative agents such as ethyl or propyl p-hydroxybenzoate, and anti-oxidants, such as ascorbic acid. Tablet formulations may be uncoated or coated either to modify their disintegration and the subsequent absorption of the active ingredient within the gastrointestinal tract, or to improve their stability and/or appearance, in either case, using conventional coating agents and procedures well known in the art.

Compositions for oral use may be in the form of hard gelatin capsules in which the active ingredient is mixed with an inert solid diluent, for example, calcium carbonate, calcium phosphate or kaolin, or as soft gelatin capsules in which the active ingredient is mixed with water or an oil such as peanut oil, liquid paraffin, or olive oil.

Aqueous suspensions generally contain the active ingredient in finely powdered form together with one or more suspending agents, such as sodium carboxymethylcellulose, methylcellulose, hydroxypropylmethylcellulose, sodium alginate, polyvinyl-pyrrolidone, gum tragacanth and gum acacia; dispersing or wetting agents such as lecithin or condensation products of an alkylene oxide with fatty acids (for example polyoxethylene stearate), or condensation products of ethylene oxide with long chain aliphatic alcohols, for example heptadecaethyleneoxycetanol, or condensation products of ethylene oxide with partial esters derived from fatty acids and a hexitol such as polyoxyethylene sorbitol monooleate, or condensation products of ethylene oxide with long chain aliphatic alcohols, for example heptadecaethyleneoxycetanol, or condensation products of ethylene oxide with partial esters derived from fatty acids and a hexitol such as polyoxyethylene sorbitol monooleate, or condensation products of ethylene oxide with partial esters derived from fatty acids and hexitol anhydrides, for example polyethylene sorbitan monooleate. The aqueous suspensions may also contain one or more preservatives (such as ethyl or propyl p-hydroxybenzoate, anti-oxidants (such as ascorbic acid), colouring agents, flavouring agents, and/or sweetening agents (such as sucrose, saccharine or aspartame).

Oily suspensions may be formulated by suspending the active ingredient in a vegetable oil (such as arachis oil, olive oil, sesame oil or coconut oil) or in a mineral oil (such as liquid paraffin). The oily suspensions may also contain a thickening agent such as beeswax, hard paraffin or cetyl alcohol. Sweetening agents such as those set out above, and flavouring agents may be added to provide a palatable oral preparation. These compositions may be preserved by the addition of an anti-oxidant such as ascorbic acid.

Dispersible powders and granules suitable for preparation of an aqueous suspension by the addition of water generally contain the active ingredient together with a dispersing or wetting agent, suspending agent and one or more preservatives. Suitable dispersing or wetting agents and suspending agents are exemplified by those already mentioned above. Additional excipients such as sweetening, flavoring and coloring agents, may also be present.

The pharmaceutical compositions of the invention may also be in the form of oil-in-water emulsions. The oily phase may be a vegetable oil, such as olive oil or arachis oil, or a mineral oil, such as for example liquid paraffin or a mixture of any of these. Suitable emulsifying agents may be, for example, naturally-occurring gums such as gum acacia or gum tragacanth, naturally-occurring phosphatides such as soya bean, lecithin, an esters or partial esters derived from fatty acids and hexitol anhydrides (for example sorbitan monooleate) and condensation products of the said partial esters with ethylene oxide such as polyoxyethylene sorbitan monooleate. The emulsions may also contain sweetening, flavoring and preservative agents.

Syrups and elixirs may be formulated with sweetening agents such as glycerol, propylene glycol, sorbitol, aspartame or sucrose, and may also contain a demulcent, preservative, flavoring and/or coloring agent.

The pharmaceutical compositions may also be in the form of a sterile injectable aqueous or oily suspension, which may be formulated according to known procedures using one or more of the appropriate dispersing or wetting agents and suspending agents, which have been mentioned above. A sterile injectable preparation may also be a sterile injectable solution or suspension in a non-toxic parenterally-acceptable diluent or solvent, for example a solution in 1,3-butanediol.

Compositions for administration by inhalation may be in the form of a conventional pressurized aerosol arranged to dispense the active ingredient either as an aerosol containing finely divided solid or liquid droplets. Conventional aerosol propellants such as volatile fluorinated hydrocarbons or hydrocarbons may be used and the aerosol device is conveniently arranged to dispense a metered quantity of active ingredient.

For further information on formulation the reader is referred to Chapter 25.2 in Volume 5 of Comprehensive Medicinal Chemistry (Corwin Hansch; Chairman of Editorial Board), Pergamon Press 1990.

The amount of active ingredient that is combined with one or more excipients to produce a single dosage form will necessarily vary depending upon the host treated and the particular route of administration. For example, a formulation intended for oral administration to humans will generally contain, for example, from 0.5 mg to 2 g of active agent compounded with an appropriate and convenient amount of excipients which may vary from about 5 to about 98 percent by weight of the total composition. Dosage unit forms will generally contain about 1 mg to about 500 mg of an active ingredient. For further information on Routes of Administration and Dosage Regimes the reader is referred to Chapter 25.3 in Volume 5 of Comprehensive Medicinal Chemistry (Corwin Hansch; Chairman of Editorial Board), Pergamon Press 1990.

The compounds of the invention described herein may be applied as a sole therapy or may involve, in addition to a compound of the invention, one or more other substances and/or treatments. Such conjoint treatment may be achieved by way of the simultaneous, sequential or separate administration of the individual components of the treatment. Where the administration is sequential or separate, the delay in administering the second component should not be such as to lose the beneficial effect of the combination. Suitable classes and substances may be selected from one or more of the following:

i) other antibacterial agents for example macrolides e.g. erythromycin, azithromycin or clarithromycin; quinolones e.g. ciprofloxacin or levofloxacin; B-lactams e.g. penicillins e.g. amoxicillin or piperacillin; cephalosporins e.g. ceftriaxone or ceftazidime; carbapenems, e.g. meropenem or imipenem etc; aminoglycosides e.g. gentamicin or tobramycin; or oxazolidinones; and/or

ii) anti-infective agents for example, an antifungal triazole e.g. or amphotericin; and/or

iii) biological protein therapeutics for example antibodies, cytokines, bactericidal/permeability-increasing protein (BPI) products; and/or

iv) efflux pump inhibitors.

Therefore, in a further aspect of the invention there is provided a compound of the formula (I), or a pharmaceutically acceptable salt thereof, and a chemotherapeutic agent selected from:

i) one or more additional antibacterial agents; and/or

ii) one or more anti-infective agents; and/or

iii) biological protein therapeutics for example antibodies, cytokines, bactericidal/permeability-increasing protein (BPI) products; and/or

iv) one or more efflux pump inhibitors.

In another embodiment, the invention relates to a method of treating a bacterial infection in an animal, such as a human, comprising administering to the animal an effective amount of a compound of formula (I), or a pharmaceutically acceptable salt thereof, and a chemotherapeutic agent selected from:

i) one or more additional antibacterial agents; and/or

ii) one or more anti-infective agents; and/or

iii) biological protein therapeutics for example antibodies, cytokines, bactericidal/permeability-increasing protein (BPI) products; and/or

iv) one or more efflux pump inhibitors.

As stated above the size of the dose required for the therapeutic or prophylactic treatment of a particular disease state will necessarily be varied depending on the host treated, the route of administration, the severity of the illness being treated, and whether or not an additional chemotherapeutic agent is administered in combination with a compound of the invention. Preferably a daily dose in the range of 1-50 mg/kg is employed. However the daily dose will necessarily be varied depending upon the host treated, the particular route of administration, the severity of the illness being treated, and whether or not an additional chemotherapeutic agent is administered in combination with a compound of the invention. Accordingly the optimum dosage may be determined by the practitioner who is treating any particular patient.

As noted above, one embodiment of the present invention is directed to treating or preventing diseases caused by bacterial infections, wherein the bacteria comprise a GyrB ATPase or topoisomerase IV ATPase enzyme. “Treating a subject with a disease caused by a bacterial infection” includes achieving, partially or substantially, one or more of the following: the reducing or amelioration of the progression, severity and/or duration of the infection, arresting the spread of an infection, ameliorating or improving a clinical symptom or indicator associated with a the infection (such as tissue or serum components), and preventing the reoccurrence of the infection.

As used herein, the terms “preventing a bacterial infection” refer to the reduction in the risk of acquiring the infection, or the reduction or inhibition of the recurrence of the infection. In a preferred embodiment, a compound of the invention is administered as a preventative measure to a patient, preferably a human, before a surgical procedure is preformed on the patient to prevent infection.

As used herein, the term “effective amount” refers to an amount of a compound of this invention for treating or preventing a bacterial infection is an amount which is sufficient to prevent the onset of an infection, reduce or ameliorate the severity, duration, or progression, of an infection, prevent the advancement of an infection, cause the regression of an infection, prevent the recurrence, development, onset or progression of a symptom associated with an infection, or enhance or improve the prophylactic or therapeutic effect(s) of another therapy.

In addition to its use in therapeutic medicine, compounds of formula (I), and their pharmaceutically acceptable salts, are also useful as pharmacological tools in the development and standardization of in-vitro and in-vivo test systems for the evaluation of the effects of inhibitors of DNA gyrase and/or topoisomerase IV in laboratory animals such as cats, dogs, rabbits, monkeys, rats and mice, as part of the search for new therapeutic agents.

In the above pharmaceutical composition, process, method, use and medicament manufacture features, the alternative and particular embodiments of the compounds of the invention described herein also apply.

### Experimental

The invention is now illustrated but not limited by the following Examples in which unless otherwise stated:
(i) evaporations were carried out by rotary evaporation in-vacuo and work-up procedures were carried out after removal of residual solids by filtration;
(ii) operations were generally carried out at ambient temperature, that is typically in the range 18-26° C. and without exclusion of air unless otherwise stated, or unless the skilled person would otherwise work under an inert atmosphere;
(iii) column chromatography (by the flash procedure) was used to purify compounds and was performed on Merck Kieselgel silica (Art. 9385) unless otherwise stated;
(iv) yields are given for illustration only and are not necessarily the maximum attainable; the structure of the end-products of the invention were generally confirmed by NMR and mass spectral techniques; proton magnetic resonance spectra is quoted and was generally determined in DMSO-d₆ unless otherwise stated using a Bruker DRX-300 spectrometer operating at a field strength of 300 MHz or a Bruker Avance-II spectrometer operating at a field strength of 400 MHz. Chemical shifts are reported in parts per million downfield from tetramethysilane as an internal standard (8 scale) and peak multiplicities are shown thus: s, singlet; d, doublet; AB or dd, doublet of doublets; dt, doublet of triplets; dm, doublet of multiplets; t, triplet, m, multiplet; br, broad; fast-atom bombardment (FAB) mass spectral data were generally obtained using a Platform spectrometer (supplied by Micromass) run in electrospray and, where appropriate, either positive ion data or negative ion data were collected or using Agilent 1100series LC/MSD equipped with Sedex 75ELSD, run in atmospheric pressure chemical ionisation mode and, where appropriate, either positive ion data or negative ion data were collected; mass spectra were run with an electron energy of 70 electron volts in the chemical ionization (CI) mode using a direct exposure probe; where indicated ionization was effected by electron impact (EI), fast atom bombardment (FAB) or electrospray (ESP); values for m/z are given; generally, only ions which indicate the parent mass are reported;
(vi) each intermediate was purified to the standard required for the subsequent stage and was characterised in sufficient detail to confirm that the assigned structure was correct; purity was assessed by high pressure liquid chromatography, thin layer chromatography, or NMR and identity was determined by infra-red spectroscopy (IR), mass spectroscopy or NMR spectroscopy as appropriate;
(vii) Where unspecified, the total amount of solvent(s) used in a given transformation was such that the concentration of the limiting substrate in the reaction mixture was between 0.1 to 0.5 Molar.
(viii) the following abbreviations may be used:

ACN is acetonitrile;

CDCl₃ is deuterated chloroform;

DBU is 1,8-diazabicyclo[5.4.0]undec-7-ene;

DCM is dichloromethane;

DIEA is diisopropyl ethylamine;

DMF is N,N-dimethylformamide;

DMSO is dimethylsulfoxide;

EDC is 1-ethyl-3-(3-dimethylaminopropyl)carbodiimide;

EtOAc is ethyl acetate;

EtOH is ethanol;

HATU is N-[(dimethylamino)-1H,2,3-triazolo]4,5-b-]pyridin-1-ylmethylene]-N-methylmethanaminium hexafluorophosphate N-oxide;

HOBT is 1-hydroxybenzotriazole;

MeOH is methanol;

MS is mass spectroscopy;

NMP is N-Methylpyrrolidone;

RT or rt is room temperature;

SM is starting material;

T₃P is n-propyl phosphonic acid cyclic anhydride

TBTU is O-(Benzotriazol-1-yl)-N,N,N′,N′-tetramethyluronium tetrafluoroborate;

TFA is trifluoroacetic acid;

TFAA is trifluoroacetic anhydride;

THF is tetrahydrofuran;
(ix) temperatures are quoted as ° C.; and
(x) vol designates 1 mL of solvent or reagent per g of material used as the limiting agent.

### Intermediates

### Intermediate 1: N'-(5-Bromo-2-chloro-pyrimidin-4-yl)-N,N-dimethyl-propane-1,3-diamine

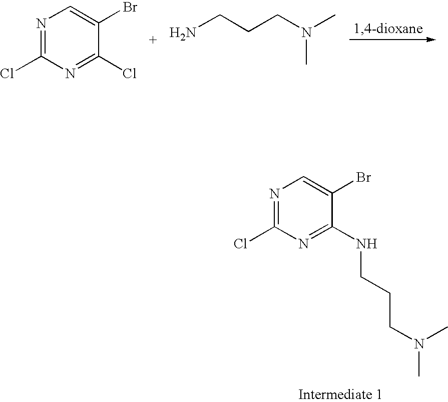

To a stirred solution of N,N-dimethyl-propane-1,3-diamine (40 mmol, 5 mL) in dioxane (40 mL) at room temperature under nitrogen atmosphere was added 5-bromo-2,4-dichloro-pyrimidine (6.6 g, 30 mmol) as a solid. Further dilution (ethyl acetate 30 mL) became necessary as the reaction progressed. The mixture was stirred overnight; the un-reacted hydrochloride salt of N,N-dimethyl-propane-1,3-diamine was removed by filtration. The filtrate was concentrated to give the title compound as a yellow solid in 86% yield (7.6 g).

MS(ES): 293.1(M) and 295.1(M+2) for C₉H₁₄BrClN₄.

¹H-NMR (400 MHz, CDCl₃): δ 1.77-1.80 (m, 2H), 2.32 (s, 6H), 2.56 (t, J=5.76 Hz, 2H), 3.60 (dt, J=9.36, 4.68 Hz, 2H), 8.05 (s, 1H), 8.7 (br s, 1H).

The following intermediates were prepared using the general method described above for Intermediate 1 using 5-bromo-2,4-dichloro-pyrimidine and the starting material (SM) indicated.

| Int | Compound | Data | SM |
|---|---|---|---|
| 2 | 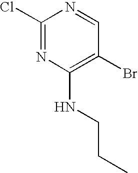 (5-Bromo-2- chloro- pyrimidin-4-yl)- propyl-amine | ¹H NMR 400 MHz, DMSO-d₆: δ 0.9 (m, 3H),1.5-1.6 (m, 2H), 3.28-3.33 (m, 2H), 7.74 (br s, 1H), 8.2 (s, 1H). | n- propylamine |
| 3 | 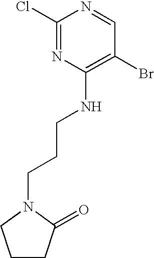 1-[3-(5-Bromo- 2-chloro- pyrimidin-4- ylamino)- propyl]- pyrrolidin-2- one | MS(ES): 333 (M) and 334 (M + 1) for C₁₁H₁₄BrClN₄O. 400 MHz, DMSO-d₆: δ 1.7 (m, 2H), 1.95 (m, 2H), 2.21 (t, J = 4 Hz, 2H), 3.2 (m, 2H), 3.39-3.27 (m, 4H), 7.73 (br s, 1H), 8.22 (s, 1H). | 1-(3-Amino- propyl)- pyrrolidin-2- one |
| 4 | 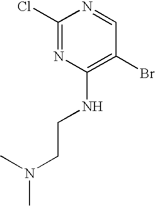 N′-(5-Bromo-2- chloro- pyrimidin-4-yl)- N,N-dimethyl- ethane-1,2- diamine | The compound wqas taken to the next step without any characterization. | N,N- dimethyl- ethane-1,2- diamine |
| 5 | 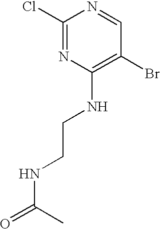 N-[2-(5-Bromo- 2-chloro- pyrimidin-4- ylamino)- ethyl]- acetamide | The compound was taken to the next step without any characterization. | N-(2-Amino- ethyl)- acetamide |
| 6 | 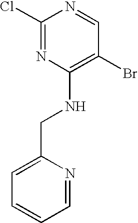 (5-Bromo-2- chloro- pyrimidin-4-yl)- (pyridin-2- ylmethyl)- amine | MS(ES): 299 (M) and 301 (M + 2) for C₁₀H₈BrClN₄. | C-Pyridin-3- yl- methylamine |
| 7 | 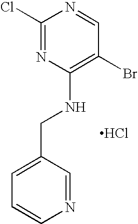 (5-Bromo-2- chloro- pyrimidin-4-yl)- (pyridin-3- ylmethyl)- amine HCl salt | The compound was taken to the next step without any characterization. | C-Pyridin-3- yl- methylamine |
| 8 | 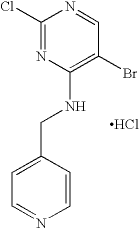 (5-Bromo-2- chloro- pyrimidin-4-yl)- (pyridin-4- ylmethyl)- amine HCl salt | The compound was taken to the next step without any characterization. | C-Pyridin-4- yl- methylamine |
| 9 | 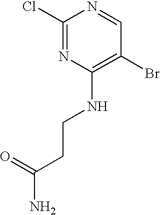 3-(5-Bromo-2- chloro- pyrimidin-4- ylamino)- propionamide | ¹H NMR 300 MHz, DMSO-d₆: δ 2.37 (t, J = 7.2 Hz, 2H), 3.50-3.56 (m, 2H), 6.91 (br s, 1H), 7.39 (br s, 1H), 7.70 (br s, 1H), 8.23 (s, 1H). | 3-Amino- propionamide |
| 10 | 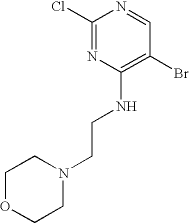 (5-Bromo-2- chloro- pyrimidin-4-yl)- (2-morpholin-4- yl-ethyl)-amine | The compound was taken to the next step without any characterization. | 2-Morpholin- 4-yl- ethylamine |
| 11 | 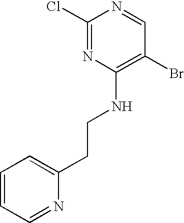 (5-Bromo-2- chloro- pyrimidin-4-yl)- (2-pyridin-2-yl- ethyl)-amine | The compound was taken to the next step without any characterization. | 2-Pyridin-2- yl-ethylamine |
| 12 | 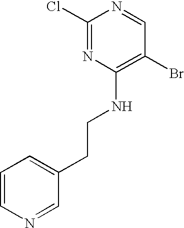 (5-Bromo-2- chloro- pyrimidin-4-yl)- (2-pyridin-3-yl- ethyl)-amine | The compound was taken to the next step without any characterization. | 2-Pyridin-3- yl-ethylamine |
| 13 | 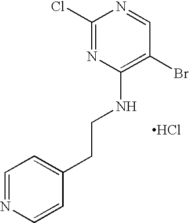 (5-Bromo-2- chloro- pyrimidin-4-yl)- (2-pyridin-4-yl- ethyl)-amine HCl salt | MS(ES): 313.59 (M) and 315 (M + 2) for C₁₁H₁₀BrClN₄. 400 MHz, DMSO-d₆: 3.18 (m, 2H), 3.74 (m, 2H), 7.8 (m, 1H), 7.95 (d, J = 6.64 Hz, 2H), 8.24 (s, 1H), 8.82 (d, J = 6.64 Hz, 2H). | 2-Pyridin-4- yl-ethylamine |
| 14 | 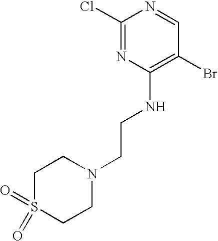 (5-Bromo-2- chloro- pyrimidin-4-yl)- [2-(1,1-dioxo- 116- thiomorpholin- 4-yl)-ethyl]- amine | MS(ES): 369.7 (M) and 371 (M + 2) for C₁₀H₁₄BrClN₄O₂S. 400 MHz, DMSO-d₆: δ 2.68 (t, J = 6.52 Hz, 2H), 2.96 (m, 4H), 3.05 (m, 4H), 3.46 (q, J = 6.36 Hz, 2H), 7.61 (t, J = 5.60 Hz, 1H), 8.24 (s, 1H). | 2-(1,1-Dioxo- 1λ⁶- thiomorpholin- 4-yl)- ethylamine |
| 15 | 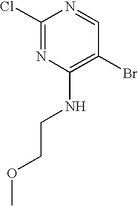 (5-Bromo-2- chloro-4- pyrimidin-4-yl)- (2-methoxy- ethyl)-amine | The compound was taken to the next step on the basis of ¹H NMR. 400 MHz DMSO-d₆: δ 1.33 (t, J = 7.1 Hz, 3H), 3.25 (s, 3H), 3.52 (q, J = 5.4 Hz, 4H), 4.36 (q, J = 7.1 Hz, 2H), 6.9 (brs, 1H), 7.29 (t, J = 9.12 Hz, 1H), 7.6 (m, 1H), 7.86 (s, 1H), 8.2 (m, J = 2.7 Hz, 2H), 8.77 (d, J = 2.2 Hz, 1H), 9.04 (d, J = 2 Hz, 1H), 9.48 (br s, 1H). | 2-Methoxy- ethylamine |
| 16 | 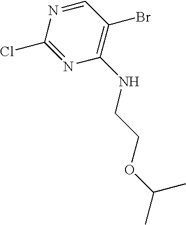 (5-Bromo-2- chloro- pyrimidin-4-yl)- (2-isopropoxy- ethyl)-amine | The compound was taken to the next step without any characterization | 2- Isopropoxy- ethylamine |
| 17 | 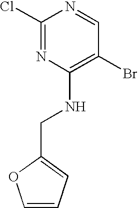 (5-Bromo-2- chloro- pyrimidin-4-yl)- (furan-2- ylmethyl)- amine | MS(ES): 288 (M) and 290 (M + 2) for C₉H₇BrClN₃O. 400 MHz, DMSO-d₆: δ 4.54 (d, J = 5.88 Hz, 1H), 6.25 (d, J = 3.16 Hz, 1H), 6.37-6.38 (m, 1H), 7.57 (t, J = 0.92 Hz, 1H), 8.23 (t, J = 5.88 Hz, 1H), 8.28 (s, 1H). | C-Furan-2- yl- methylamine |
| 18 | 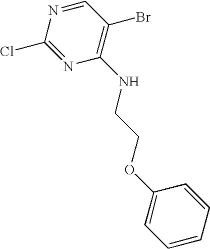 (5-Bromo-2- chloro- pyrimidin-4-yl)- (2-phenoxy- ethyl)-amine | The compound was taken to the next step without any characterization. | 2-Phenoxy- ethylamine |
| 19 | 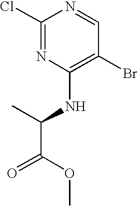 Methyl (2R)-2- [(5-bromo-2- chloropyrimidin- 4- yl)amino]pro- panoate | MS(ES): 294 (M) and 296 (M + 2) for C₈H₉BrClN₃O₂. 400 MHz, CDCl₃ δ: 1.55 (d, J = 7.08 Hz, 3H), 3.81 (s, 3H), 4.81 (m, 1H), 6.14 (d, J = 6.16 Hz, 1H), 8.18 (s, 1H). | 2R-Amino- propionic acid methyl ester |
| 20 | 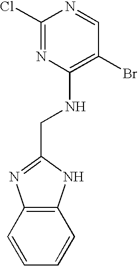 (1H-Benzoimidazol- 2-ylmethyl)-(5- bromo-2- chloro- pyrimidin-4-yl)- amine | MS(ES): 338 (M) and 340 (M + 2) for C₁₂H₉BrClN₅. 300 MHz, DMSO-d₆: δ 4.79 (d, J = 5.79 Hz, 2H),7.13 (m, 2H), 7.47 (d, J = 3.8 Hz, 2H), 8.3 (m, 1H), 12.24 (br s, 1H). | C-(1H- Benzoimidaz ol-2-yl)- methylamine |
| 21 | 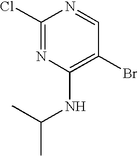 (5-Bromo-2- chloro- pyrimidin-4-yl)- isopropyl-amine | 300 MHz, DMSO-d₆: δ 1.18 (d, J = 6.6 Hz, 6H), 4.24 (m, 1H), 7.31 (d, J = 7.5 Hz, 1H), 8.21 (s, 1H). | Isopropyl- amine |
| 22 | 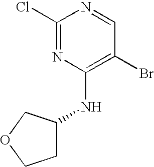 MS(ES): 279 (M + H) for C₈H₉BrClN₃O. 300 MHz, CDCl₃ δ: 1.11 (m, 1H), 2.33 (m, 1H), 3.69-4.00 (m, 4H), 4.68 (br. m, 1H), 5.56 (br. s, 1H), 8.08 (s, 1H). | (3R)-oxolan- 3-amine | |
| 23 | 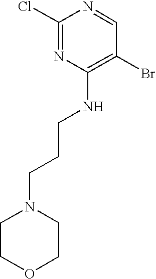 (5-Bromo-2- chloro- pyrimidin-4-yl)- (3-morpholin-4- yl-propyl)- amine | The compound was taken to the next step without any characterization. | 3-Morpholin- 4-yl- propylamine |
| 24 | 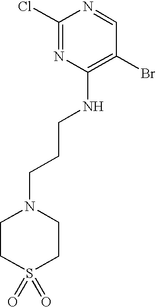 (5-Bromo-2- chloro- pyrimidin-4-yl)- [3-(1,1-dioxo- 116- thiomorpholin- 4-yl)-propyl]- amine | MS(ES): 383 (M) and 385 (M + 2) for C₁₁H₁₆BrClN₄O₂S. 400 MHz, CD₃OD: δ 1.79-1.86 (m, 2H), 2.62 (t, J = 6.56 Hz, 2H), 3.01- 3.03 (m, 4H), 3.12-3.14 (m, 4H), 3.58 (t, J = 6.96 Hz, 1H), 8.12 (s, 1H). | 3-(1,1-Dioxo- 1λ⁶- thiomorpholin- 4-yl)- propylamine |

The following intermediates were prepared using the general method described above for Intermediate 1 using the starting materials (SM) indicated.

| Int | Compound | Data | SM |
|---|---|---|---|
| 25 | 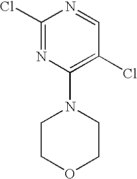 4-(2,5- dichloropyridin-4- yl)morpholine | MS: ES+ 234 for C₈H₉Cl₂N₃O 1H NMR (300 MHz, DMSO-d₆) δ ppm 3.60-3.81 (m, 8 H) 8.32 (s, 1 H) | 2,4,5- trichloro pyrimidine and morpholine |

### Intermediate 26: 5-Bromo-N²-(3-chloro-4-fluoro-phenyl)-N⁴-(3-dimethylamino-propyl)-pyrimidine-2,4-diamine

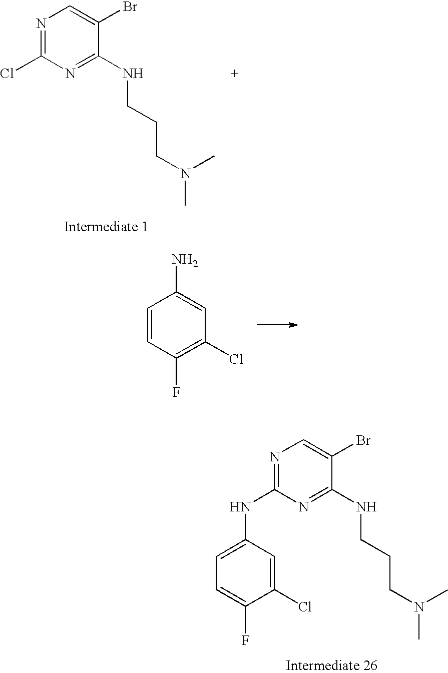

A solution of N′-(5-Bromo-2-chloro-pyrimidin-4-yl)-N,N-dimethyl-propane-1,3-diamine (Intermediate 1, 10 g, 34 mmol), 3-chloro-4-fluoroaniline (34 mmol, 4.95 g) and 2.6 M HCl in 1,4-dioxane (40 mL) was warmed to 100° C. with constant stirring. The reaction was monitored by TLC. Upon completion of reaction, the mixture was cooled to room temperature. The white solid was filtered off, washed with n-butanol and ether to afford the product as a fluffy white solid in 73% yield (24 mmol; 10 g).

MS(ES): 402 (M) and 404 (M+2) for C₁₅H₁₈BrClFN₅

¹H-NMR (400 MHz, DMSO-d6): 1.95-1.98 (m, 2H), 2.72 (s, 3H), 2.73 (s, 3.03-3.07 (m, 2H), 3.43-3.48 (m, 2H), 7.37 (t, J=9.20 Hz, 1H), 7.54 (m, 1H), 8.02 (dd, J=6.80, 2.40 Hz, 1H), 8.13 (s, 1H), 9.80 (s, 1H), 10 (s, 1H).

The following intermediates were prepared using the general method described above for Intermediate 26 using N′-(5-Bromo-2-chloro-pyrimidin-4-yl)-N,N-dimethyl-propane-1,3-diamine Intermediate 1 and the starting material (SM) indicated.

| Int | Compound | Data | SM |
|---|---|---|---|
| 27 | 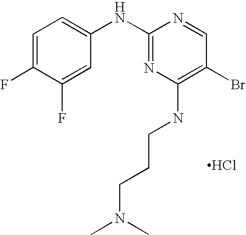 5-Bromo-N²-(3,4- difluoro-phenyl)-N⁴- (3-dimethylamino- propyl)-pyrimidine- 2,4-diamine hydrochloride salt | MS(ES): 388 (M + 2) for C₁₅H₁₈BrF₂N₅. 400 MHz, DMSO-d₆: δ 1.99 (t, J = 7.4 Hz, 2H), 2.69 (s, 3H), 2.70 (s, 3H), 3.02 (m, 2H), 3.47-3.48 (m, 2H), 7.36-7.38 (m, 1H), 7.42- 7.47 (m, 1H), 7.80 (ddd, J = 14.86, 6.58, 3.40 Hz, 1H), 8.24 (br s, 2H), 10.5 (br s, 1H), 10.7 (br s, 1H). | 3,4-Difluoro- phenylamine |
| 28 | 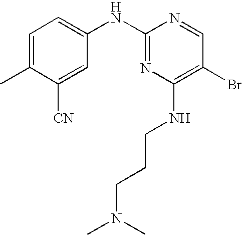 5-[5-Bromo-4-(3- dimethylamino- propylamino)- pyrimidin-2- ylamino]-2-methyl- benzonitrile | MS(ES): 389 (M) for C₁₇H₂₁BrN₆. 400 MHz, CD₃OD: δ 1.91-1.95 (m, 2H), 2.41 (s, 6H), 2.47 (s, 3H), 2.64-2.67 (m, 2H), 3.56 (t, J = 8 Hz, 2H), 7.29 (d, J = 8.0 Hz, 1H), 7.6 (dd, J = 4.0, 8.0 Hz, 1H), 7.95 (s, 1H), 8.29 (d, 1H). | 5-Amino-2- methyl- benzonitrile |
| 29 | 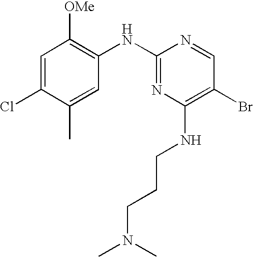 5-Bromo-N²-(4- chloro-2-methoxy-5- methyl-phenyl)-N⁴- (3-dimethylamino- propyl)-pyrimidine- 2,4-diamine | MS(ES): 428 (M) for C₁₇H₂₃BrClN₅O. 400 MHz, DMSO-d₆: δ 1.60-1.80 (m, 2H), 2.14 (s, 6H), 2.25 (s, 3H), 2.30-2.40 (m, 2H), 3.40-3.50 (m, 2H), 3.83 (s, 3H), 7.04 (s, 1H), 7.5 (br s, 1H), 7.60 (s, 1H), 7.99 (s, 1H), 8.19 (s, 1H). | 4-Chloro-2- methoxy-5- methyl- phenylamine |
| 30 | 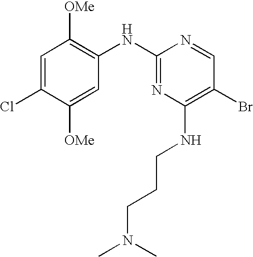 5-Bromo-N²-(4- chloro-2,5- dimethoxy-phenyl)- N⁴-(3- dimethylamino- propyl)-pyrimidine- 2,4-diamine | MS(ES): 444.7 (M) for C₁₇H₂₃BrClN₅O₂. 400 MHz, DMSO-d₆: δ 1.70-1.73 (m, 2H), 2.20 (s, 6H), 2.25 (s, 3H), 2.30-2.40 (m, 2H), 3.46-3.47 (m, 2H), 3.80 (s, 3H), 3.82 (s, 3H), 7.08 (s, 1H), 7.61 (br s, 1H), 8.02 (s, 1H), 8.19 (s, 1H). | 4-Chloro-2,5- dimethoxy- phenylamine |
| 31 | 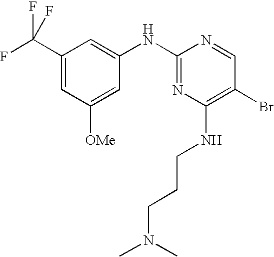 5-Bromo-N⁴-(3- dimethylamino- propyl)-N²-(3- methoxy-5- trifluoromethyl- phenyl)-pyrimidine- 2,4-diamine | MS(ES): 448 (M) and 450 (M + 2) for C₁₇H₂₁BrF₃N₅O 400 MHz, CD₃OD: δ 2.08-2.12 (m, 2H), 2.87 (s, 6H), 3.14-3.18 (m, 2H), 3.67 (t, J = 6.64 Hz, 2H), 3.92 (s, 3H), 7.13 (s, 1H), 7.38 (t, J = 1.84 Hz, 1H), 7.55 (s, 1H), 8.15 (s, 1H). | 3-Methoxy- 5- trifluoromethyl- phenylamine |
| 32 | 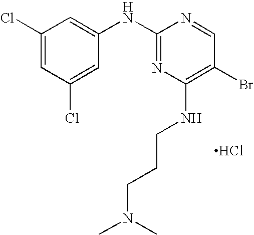 5-Bromo-N²-(3,5- dichloro-phenyl)-N⁴- (3-dimethylamino- propyl)-pyrimidine- 2,4-diamine hydrochloride salt | MS(ES): 418 (M) and 420 (M + 2) for C₁₅H₁₈BrCl₂N_{5.} 400 MHz, DMSO-d6: δ 1.97- 2.04 (m, 2H), 2.71 (s, 6H), 3.03- 3.08 (br s, 2H), 3.46-3.51 (m, 2H), 7.13 (d, J = 1.2 Hz, 1H), 7.65 (br s, 1H), 7.82 (d, J = 1.6 Hz, 1H), 8.16 (d, J = 1.2 Hz, 1H), 10.0 (br s, 1H), 10.4 (br s, 1H). | 3,5-Dichloro- phenylamine |
| 33 | 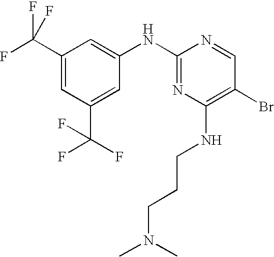 N²-(3,5-Bis- trifluoromethyl- phenyl)-5-bromo-N⁴- (3-dimethylamino- propyl)-pyrimidine- 2,4-diamine | MS(ES): 486.4 (M) for C₁₇H₁₈BrF₆N₅. 400 MHz, DMSO-d6: δ 1.69-1.76 (m, 2H), 2.15 (s, 6H), 2.34 (t, J = 6.4 Hz, 2H), 3.48 (t, J = 6.4 Hz, 2H), 7.52 (s, 1H), 7.73 (t, J = 4.8 Hz, 1H), 8.1 (s, 1H), 8.47 (s, 2H), 9.96 (s, 1H). | 3,5-Bis- trifluoromethyl- phenylamine |
| 34 | 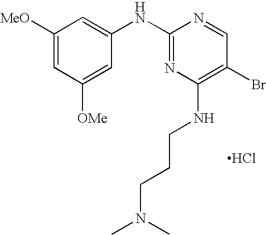 5-Bromo-N²-(3,5- dimethoxy-phenyl)- N⁴-(3- dimethylamino- propyl)-pyrimidine- 2,4-diamine hydrochloride salt | MS(ES): 410 (M) and 412 (M + 2) for C₁₇H₂₄BrN₅O₂. 400 MHz, CD₃OD: δ 2.09-2.13 (m, 2H), 2.88 (s, 6H), 3.16-3.20 (m, 2H), 3.65 (t, J = 6.8 Hz, 2H), 3.83 (s, 6H), 6.49 (t, J = 2.2 Hz, 1H), 6.69 (d, J = 2.2 Hz, 2H), 8.05 (s, 1H). | 3,5- Dimethoxy- phenylamine |

The following intermediates were prepared using the general method described above for Intermediate 26 using 3-chloro-4-fluoroaniline and the starting material (SM) indicated.

| Int | Compound | Data | SM |
|---|---|---|---|
| 35 | 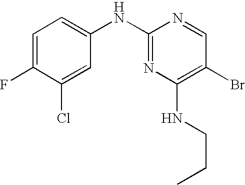 5-Bromo-N²-(3- chloro-4-fluoro- phenyl)-N⁴-propyl- pyrimidine-2,4- diamine | MS(ES): 359. (M) and 360 (M + 1) for. C₁₃H₁₃BrClFN₄ 400 MHz, DMSO-d₆: δ 0.91 (t, J = 7.48 Hz, 3H), 1.55-1.64 (m, 2H), 3.34-3.38 (m, 2H), 7.13 (t, J = 5.72 Hz, 1H), 7.28 (t, J = 9.08 Hz, 1H), 7.52 (ddd, J = 9.06, 4.26, 2.68 Hz, 1H), 8.02 (s, 1H), 8.116 (dd, J = 6.92, 2.64 Hz, 1H), 9.43 (s, 1H). | (5-Bromo-2- chloro- pyrimidin-4-yl)- propyl-amine (Intermediate 2) |
| 36 | 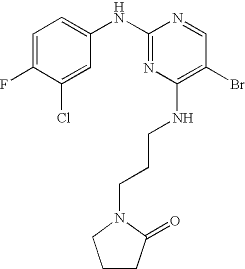 1-{3-[5-Bromo-2-(3- chloro-4-fluoro- phenylamino)- pyrimidin-4-ylamino]- propyl}-pyrrolidin-2- one | MS(ES) 442.8 (M) for C₁₇H₁₈BrClFN₅O. 400 MHz, DMSO-d₆: δ 1.74- 1.79 (m, 2H), 1.84-1.91 (m, 2H), 2.18 (t, J = 8.16 Hz, 2H), 3.21 (t, J = 6.80 Hz, 2H), 3.30 (t, J = 7.00 Hz, 2H), 3.36-3.57 (m, 2H), 7.43 (t, J = 9.00 Hz, 1H), 7.50 (ddd, J = 8.90, 4.22, 2.84 Hz, 1H), 7.95 (dd, J = 6.72, 2.48 Hz, 1H), 8.17- 8.21 (m, 1H), 8.23 (s, 1H), 10.44 (br s, 1H). | 1-[3-(5-Bromo- 2-chloro- pyrimidin-4- ylamino)- propyl]- pyrrolidin-2-one (Intermediate 3) |
| 37 | 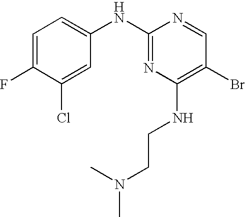 5-Bromo-N²-(3- chloro-4-fluoro- phenyl)-N⁴-(2- dimethylamino-ethyl)- pyrimidine-2,4- diamine | MS(ES): 388 (M) and 390 (M + 2) for C₁₄H₁₆BrClFN₅. 400 MHz, MeOD: δ 2.90 (s, 6H), 3.40 (t, J = 5.9 Hz, 2H), 3.89 (t, J = 5.96 Hz, 2H), 7.38 (t, J = 8.84 Hz, 1H), 7.42-7.45 (m, 1H), 7.72 (dd, J = 2.52, 6.52 Hz, 1H), 8.13 (s, 1H). | N′-(5-Bromo-2- chloro- pyrimidin-4-yl)- N,N-dimethyl- ethane-1,2- diamine (Intermediate 4) |
| 38 | 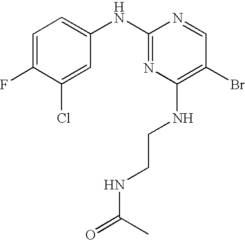 N-{2-[5-Bromo-2-(3- chloro-4-fluoro- phenylamino)- pyrimidin-4-ylamino]- ethyl}-acetamide | MS(ES): 402 (M) and 404 (M + 2) for C₁₄H₁₄BrClFN₅O 400 MHz, DMSO-d₆: δ 1.79 (s, 3H), 3.29 (m, 2H), 3.46 (q, J = 5.8 Hz, 2H), 7.1 (t, J = 5.28 Hz, 1H), 7.29 (t, J = 9.16 Hz, 1H), 7.61-7.67 (m, 1H), 7.96 (t, J = 5.52 Hz, 1H), 8.02 (dd, J = 6.88, 2.64 Hz, 1H), 8.06 (s, 1H), 9.44 (s, 1H). | N-[2-(5-Bromo- 2-chloro- pyrimidin-4- ylamino)-ethyl]- acetamide (Intermediate 5) |
| 39 | 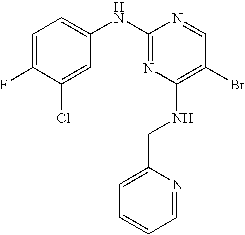 5-Bromo-N²-(3- chloro-4-fluoro- phenyl)-N⁴-pyridin-2- ylmethyl-pyrimidine- 2,4-diamine | MS(ES): 408.9 (M + 1) and 410 (M + 2) for C₁₆H₁₂BrClFN₅. 400 MHz, DMSO-d₆: δ 4.71 (d, J = 5.84 Hz, 2H), 7.15 (t, J = 9.12 Hz, 1H), 7.23-7.27 (m, 2H), 7.43 (m, 1H), 7.69-7.76 (m, 2H), 7.86 (dd, J = 6.76, 2.56 Hz, 1H), 8.11 (s, 1H), 8.54 (ddd, J = 4.74, 1.64, 0.84 Hz, 1H), 9.41 (s, 1H). | (5-Bromo-2- chloro- pyrimidin-4-yl)- (pyridin-2- ylmethyl)-amine (Intermediate 6) |
| 40 | 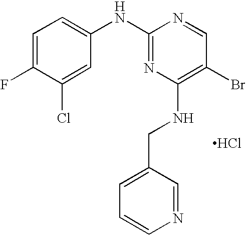 5-Bromo-N²-(3- chloro-4-fluoro- phenyl)-N⁴-(pyridin- 3-ylmethyl)- pyrimidin-2,4- diamine hydrochloride salt | MS(ES): 410 (M + 2) for C₁₆H₁₂BrClFN_{5.} 400 MHz, DMSO-d₆: 4.79 (d, J = 4 Hz, 2H), 7.3-7.34 (m, 1H), 7.38-7.4 (m, 1H), 7.70-7.72 (br s, 1H), 8.02 (dd, J = 8, 4 Hz, 1H), 8.28 (s, 1H), 8.5 (d, J = 4 Hz, 1H), 8.62 (br s, 1H), 8.82 (d, J = 4 Hz, 1H), 8.93 (s, 1H), 10.36 (br s, 1H). | (5-Bromo-2- chloro- pyrimidin-4-yl)- (pyridin-3- ylmethyl)-amine HCl salt (Intermediate 7) |
| 41 | 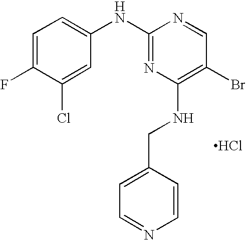 5-Bromo-N²-(3- chloro-4-fluoro- phenyl)-N⁴-pyridin-4- ylmethyl-pyrimidine- 2,4-diamine hydrochloride salt | MS(ES): 408.6 (M) and 409.8 (M + 2) for C₁₆H₁₂BrClFN_{5.} 400 MHz, DMSO-d₆: 4.84 (d, J = 5.84 Hz, 2H), 7.2 (t, J = 9.08 Hz, 1H), 7.3 (m, 1H), 7.70 (m, 1H), 7.91 (d, J = 6.56 Hz, 2H), 8.05 (s, 1H), 8.19 (s, 1H), 8.81 (d, J = 6.64 Hz, 2H), 9.58 (s, 1H). | (5-Bromo-2- chloro- pyrimidin-4-yl)- (pyridin-4- ylmethyl)-amine HCl salt (Intermediate 8) |
| 42 | 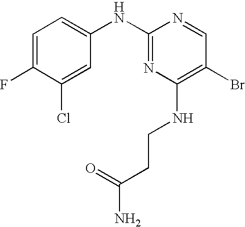 3-[5-Bromo-2-(3- chloro-4-fluoro- phenylamino)- pyrimidin-4-ylamino]- propionamide | MS(ES): 388.8 (M) 389.8 (M + 1) for C₁₃H₁₂BrClFN₅O. 400 MHz, DMSO-d₆: δ 2.42 (t, J = 7 Hz, 2H), 3.59 (q, J = 6.84 Hz, 2H), 6.89 (br s, 1H), 7.01 (t, J = 5.64 Hz, 1H), 7.26 (t, J = 9.12 Hz, 1H), 7.35 (s, 1H), 7.68 (ddd, J = 9.06, 4.18, 2.72 Hz, 1H), 8.03 (m, 2H), 9.46 (s, 1H). | 3-(5-Bromo-2- chloro- pyrimidin-4- ylamino)- propionamide (Intermediate 9) |
| 43 | 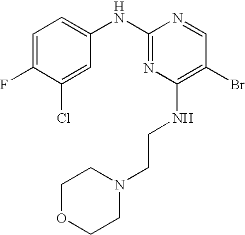 5-Bromo-N²-(3- chloro-4-fluoro- phenyl)-N⁴-(2- morpholin-4-yl- ethyl)-pyrimidin-2,4- diamine | MS(ES): 430 (M) and 432 (M + 2) for C₁₆H₁₈BrClFN₅O. 400 MHz, DMSO-d₆: δ 2.40 (br s, 4H), 2.48-2.53 (m, 2H), 3.48- 3.56 (m, 6H), 6.92 (t, J = 5.44 Hz, 1H), 7.27 (t, J = 9.08 Hz, 1H), 7.57 (ddd, J = 9.06, 4.26, 2.68 Hz, 1H), 8.05 (m, 2H), 9.42 (s, 1H). | (5-Bromo-2- chloro- pyrimidin-4-yl- (2-morpholin-4- yl-ethyl)-amine (Intermediate 10) |
| 44 | 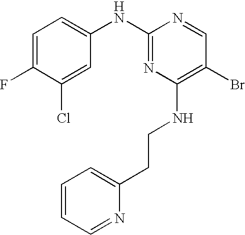 5-Bromo-N²-(3- chloro-4-fluoro- phenyl)-N⁴-(2- pyridin-2-yl-ethyl)- pyrimidine-2,4- diamine | MS(ES): 424 (M + 1) for C₁₇H₁₄BrClFN₅. 400 MHz, MeOD: δ 3.39 (t, J = 6.4 Hz, 2H), 3.95 (t, J = 6.44 Hz, 2H), 7.36 (t, J = 8.84 Hz, 1H), 7.38 (m, 1H), 7.6 (dd, J = 2.52, 6.56 Hz, 1H), 7.9 (d, J = 8 Hz, 1H), 7.95 (t, J = 7.24 Hz, 1H), 8.12 (s, 1H), 8.45 (dt, J = 10.97, 1.36 Hz,1H), 8.74 (d, J = 5.8 Hz, 1H). | (5-Bromo-2- choloro- pyrimidin-4-yl)- (2-pyridin-2-yl- ethyl)-amine (Intermediate 11) |
| 45 | 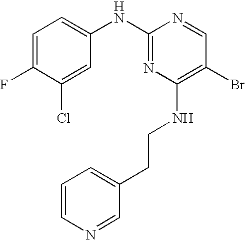 5-Bromo-N²-(3- chloro-4-fluoro- phenyl)-N⁴-(2- pyridin-3-yl-ethyl)- pyrimidine-2,4- diamine | MS(ES): 422 (M) and 424 (M + 2) for C₁₇H₁₄BrClFN_{5.} 400 MHz, DMSO-d₆: δ 3.13 (t,J = 4 Hz, 2H), 3.70-3.75 (m, 2H), 7.39 (m, 1H), 7.48-7.52 (m, 1H), 7.95-8.01 (m, 3H), 8.22 (s, 1H), 8.39 (d, J = 8 Hz, 2H), 8.79 (d, J = 8 Hz, 2H), 8.83 (d, 2H), 10.33 (br s, 1H). | (5-Bromo-2- chloro- pyrimidin-4-yl- (2-pyridin-3-yl- ethyl)-amine (Intermediate 12) |
| 46 | 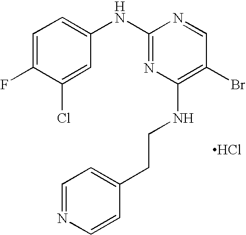 5-Bromo-N²-(3- chloro-4- phenyl)-N⁴-(2- pyridin-4-yl-ethyl)- pyrimidin-2,4- diamine hydrochloride salt | MS(ES): 422.7 (M) and 424 (M + 1) for. C₁₇H₁₄BrClFN₅. 400 MHz, DMSO-d₆: δ 3.21 (t, J = 6.80 Hz, 2H), 3.75-3.77 (m, 2H), 7.33 (m, 1H), 7.50 (m, 1H), 7.60 (br s, 1H), 7.89 (d, J = 6.12 Hz, 2H), 8.01 (dd, J = 6.82, 2.60 Hz, 1H), 8.13 (s, 1H), 8.81 (d, J = 6.44 Hz, 2H), 9.90 (br s,1H). | (5-Bromo-2- chloro- pyrimidin-4-yl)- (2-pyridin-4-yl- ethyl)-amine HCl salt (Intermediate 13) |
| 47 | 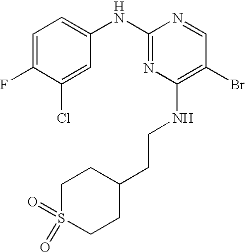 5-Bromo-N²-(3- chloro-4-fluoro- phenyl)-N⁴-[2-(1,1- dioxo-1λ⁶- thiomorpholin-4-yl)- ethyl]-pyrimidine-2,4- diamine | MS(ES): 478 (M) and 480 (M + 2) for C₁₆H₁₈BrClFN₅O₂S. 400 MHz, DMSO-d₆: δ 2.72 (t, J = 6.68 Hz, 2H), 2.96-2.97 (m, 4H), 3.03-3.04 (m, 4H), 3.48- 3.52 (m, 2H), 6.95 (t, J = 5.32 Hz, 1H), 7.29 (t, J = 9.08 Hz, 1H), 7.57 (ddd, J = 9.10, 4.24, 2.72Hz, 1H), 8.03-8.05 (m, 2H), 9.40 (s, 1H), | (5-Bromo-2- chloro- pyrimidin-4-yl)- [2-(1,1-dioxo- 116- thiomorpholin-4- yl)ethyl]-amine (Intermediate 14) |
| 48 | 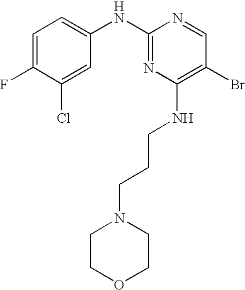 5-Bromo-N²-(3- chloro-4-fluoro- phenyl)-N⁴-(3- morpholin-4-yl- propyl)-pyrimidine- 2,4-diamine | MS(ES): 444 (M) and 446 (M + 2) for C₁₇H₂₀BrClFN₅O. 400 Mhz, DMSO-d₆: δ 1.72- 1.77 (m, 2H), 2.33-2.38 (m, 6H), 3.42-3.46 (m, 2H), 3.55-3.57 (t, J = 9.04 Hz, 4H), 7.22-7.30 (m, 2H), 7.53 (ddd, J = 8.98, 4.16, 2.76 Hz, 1H), 8.02 (s, 1H), 8.13 (dd, J = 6.88, 2.60 Hz, 1H), 9.43 (s, 1H). | (5-Bromo-2- chloro- pyrimidin-4-yl- (3-morpholin-4- yl-propyl)-amine (Intermediate 23) |
| 49 | 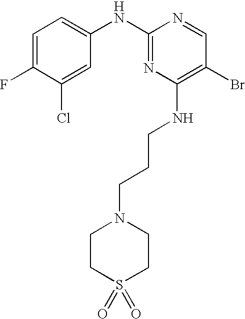 5-Bromo-N²-(3- chloro-4-fluoro- phenyl)-N⁴-[3-(1,1- dioxo-1λ⁶- thiomorpholin-4-yl)- propyl]-pyrimidine- 2,4-diamine | MS(ES): 492 (M) and 494 (M + 2) for C₁₇H₂₀BrClFN₅O₂S. 400 MHz, DMSO-d₆: δ 1.71- 1.76 (m, 2H), 2.82 (m, 4H), 2.87 (m, 4H), 3.45 (q, J = 6.28 Hz, 2H), 7.17 (t, J = 5.56 Hz, 1H), 7.3 (t, J = 9.08 Hz, 1H), 7.54 (ddd, J = 9.08, 4.22, 2.8 Hz, 1H), 8.04 (s, 1H), 8.13 (dd, J = 6.92, 2.56 Hz, 1H), 9.45 (s, 1H). | (5-Bromo-2- chloro- pyrimidin-4-yl- [3-(1,1-dioxo- 116- thiomorpholin-4- yl)-propyl]- amine (Intermediate 24) |
| 50 | 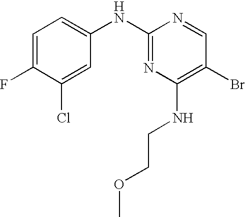 5-Bromo-N²-(3- chloro-4-fluoro- phenyl)-N⁴-(2- methoxy-ethyl)- pyrimidin-2,4- diamine | The compound was taken to the next step on the basis of ¹H NMR. 300 MHz DMSO-d₆: δ 3.3 (m, 3H), 3.5 (m, 4H), 7.34-7.36 (m, 2H), 7.9 (m, 2H), 8.2 (s, 1H), 10.3 (s, 1H). | (5-Bromo-2- chloro- pyrimidin-4-yl)- (2-methoxy- ethyl)-amine (Intermediate 15) |
| 51 | 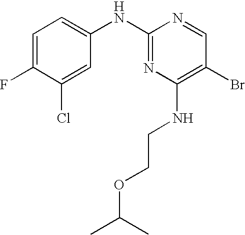 5-Bromo-N²-(3- chloro-4-fluoro- phenyl)-N⁴-(2- isopropoxy-ethyl)- pyrimidine-2,4- diamine | MS(ES): 403 (M) and 405 (M + 2) for C₁₅H₁₇BrClFN₅O. 300 MHz DMSO-d₆: δ 1.0 (dd, J = 6.18, 20.04 Hz, 6 H), 3.55 (br s, 5H), 6.93 (s, 1H), 7.27 (t, 9.12 Hz, 1H), 7.55 (ddd, J = 2.67, 4.17, 9.09 Hz, 1H), 8.04 (s, 1H), 8.07 (dd, J = 2.64, 6.87 Hz, 1H), 9.44 (s, 1H). | (5-Bromo-2- chloro- pyrimnidin-4-yl)- (2-isopropoxy- ethyl)-amine (Intermediate 16) |
| 52 | 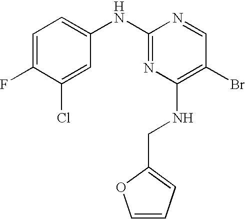 5-Bromo-N2-(3- chloro-4-fluoro- phenyl)-N4-(furan-2- ylmethyl)-pyrimidine- 2,4-diamine | MS(ES): 396.9 (M) and 398.9 (M + 2) for C₁₅H₁₁BrClFN₄O. 400 MHz DMSO-d₆: δ 4.59 (d, J = 5.88 Hz, 2H), 6.22 (d, J = 2.88 Hz, 1H), 6.37 (dd, J = 3.16, 1.84 Hz, 1H), 7.35 (t, J = 9.04 Hz, 1H), 7.48 (ddd, J = 8.96, 4.26, 2.72 Hz, 1H), 7.58 (dd, J = 1.78, 0.80 Hz, 1H), 7.95 (dd, J = 6.76, 2.56 Hz,1H), 8.43 (br s, 1H). | (5-Bromo-2- chloro- pyrimidin-4-yl)- (furan-2- ylmethyl)-amine (Intermediate 17) |
| 53 | 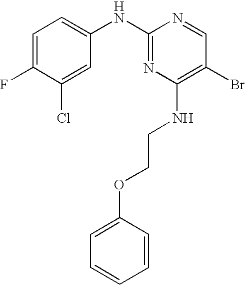 5-Bromo-N²-(3- chloro-4-fluoro- phenyl)-N⁴-(2- phenoxy-ethyl)- pyrimidine-2,4- diamine | MS(ES): 437 (M) and 439.1 (M + 2) for C₁₈H₁₅BrClFN₄O. 400 MHz) DMSO-d₆: δ 3.77 (q, J = 6.08 Hz, 2H), 4.14 (t, J = 6.24 Hz, 2H), 6.9 (m, 3H), 7.22 (m, 4H), 7.53 (ddd, J = 9.08, 4.2, 2.72 Hz, 1H), 8.07 (dd, J = 7.2, 3.2 Hz, 2H), 9.46 (s, 1H). | (5-Bromo-2- chloro- pyrimidin-4-yl)- (2-phenoxy- ethyl)-amine (Intermediate 18) |
| 54 | 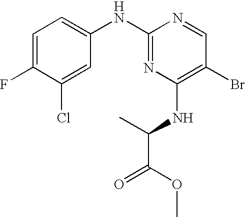 methyl (2R)-2-[[2-[(3- chloro-4- fluorophenyl)amino]- 5-bromo-pyrimidin-4- yl]amino]propanoate | MS(ES): 403 (M) and 404.8 (M + 2) for C₁₄H₁₃BrClFN₄O₂. 400 MHz, DMSO-d₆: δ 1.49 (d, J = 7.28 Hz, 3H), 3.60 (s, 3H), 4.69 (m, 1H), 7.11 (d, J = 7.36 Hz, 1H), 7.27 (t, J = 9.08 Hz, 1H), 7.52 (ddd, J = 9.06, 4.18, 2.72 Hz, 1H), 7.93 (dd, J = 6.80, 2.56 Hz, 1H), 8.12 (s, 1H), 9.47 (s, 1H) | methyl (2R)-2- [(5-bromo-2- chloropyrimidin- 4-yl)amino]- propanoate (Intermediate 19) |
| 55 | 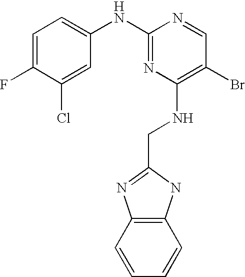 N⁴-(1H- Benzoimidazol-2- ylmethyl)-5-bromo- N²-(3-chloro-4-fluoro- phenyl)-pyrimidine- 2,4-diamine | MS(ES): 447 (M) and 449 (M + 2) for C₁₈H₁₃BrClFN₆. 300 MHz, DMSO-d₆: δ 5.06 (d, J = 5.19 Hz, 2H), 7.17 (t, J = 9.09 hz, 1H), 7.42 (m, 1H), 7.51 (dd, J = 3.16, 6.15 Hz, 2H), 7.63 (br s, 1H), 7.76 (dd, J = 3.12, 6.12 Hz, 2H), 8.11 (br s, 1H), 8.24 (s, 1H), 9.84 (s, 1H). | (1H- Benzoimidazol- 2-ylmethyl)-(5- bromo-2-chloro- pyrimidin-4-yl)- amine (Intermediate 20) |
| 56 | 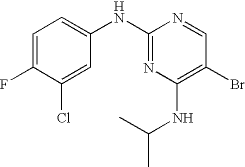 5-Bromo-N²-(3- chloro-4-fluoro- phenyl)-N⁴-isopropyl- pyrimidine-2,4- diamiune | MS(ES): 359 (M) and 360 (M + 1) for C₁₃H₁₃BrClFN₄. 300 MHz, DMSO-d₆: δ 1.23 (d, J = 6.54 Hz, 6H), 4.28 (m, 1H), 6.56 (d, J = 7.95 Hz, 1H), 7.28 (t, J = 9.12 Hz, 1H), 7.51 (td, J = 4.11, 7.65 Hz, 1H), 8.04 (s, 1H), 8.15 (dd, J = 2.61, 6.9 Hz, 1H), 9.44 (s, 1H). | (5-Bromo-2- chloro- pyrimidin-4-yl)- isopropyl-amine (Intermediate 21) |

The following intermediates were prepared using the general method described above for Intermediate 26 using the starting materials (SM) indicated

| Int | Compound | Data | SM |
|---|---|---|---|
| 57 | 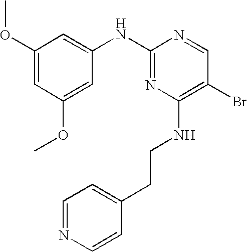 5-bromo-N²-(3,5- dimethoxyphenyl)-N⁴-(2- (pyridin-4-yl)ethyl) pyrimidin-2,4-diamine | MS: ES+ 431 for C₁₉H₂₀BrN₅O₂ 1H NMR (300 MHz, DMSO- d₆) δ ppm 3.20 (t, J = 6.78 Hz, 2 H) 3.70 (s, 6 H) 3.77 (q, J = 6.47 Hz, 2 H) 6.28 (t, J = 1.98 Hz, 1 H) 6.80 (d, J = 2.07 Hz, 2 H) 7.88 (d, J = 6.22 Hz, 2 H) 8.29 (s, 1 H) 8.36 (br. s., 1 H) 8.81 (d, J = 6.22 Hz, 2 H) 10.51 (br. s., 1 H) | Intermediate 12 and 3,5- dimethoxy aniline |
| 58 | 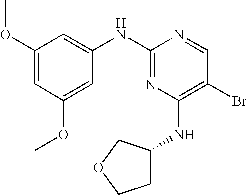 (R)-5-bromo-N²-(3,5- dimethoxyphenyl)-N⁴- (tetrahydrofuran-3-yl) pyrimidin-2,4-diamine | MS: ES+ 396 for C₁₆H₁₉BrN₄O₃ 1H NMR (300 MHz, DMSO- d₆) δ 2.02-2.28 (m, 2 H) 3.63-3.78 (m, 7 H) 3.83- 3.94 (m, 2 H) 4.55-4.69 (m, J = 12.67, 6.26, 6.08, 6.08 Hz, 2 H) 6.28 (t, 1 H) 6.83 (d, J = 2.26 Hz, 2 H) 8.02 (br. s., 1 H) 8.26 (s, 1 H) 10.34 (br. s., 1 H) | Intermediate 22 and 3,5- dimethoxy aniline |
| 59 | 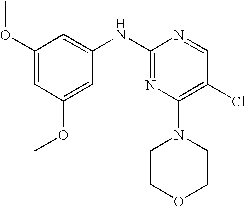 5-chloro-N-(3,5- dimethoxyphenyl)-4- morpholinopyrimidin-2- amine | MS: ES+ 351 for C₁₆H₁₉ClN₄O₃ 1H NMR (300 MHz, DMSO- d₆) δ ppm 3.60-3.77 (m, J = 3.96 Hz, 14 H) 6.16 (s, 1 H) 6.89 (d, J = 2.07 Hz, 2 H) 8.19 (s, 1 H) 9.85 (s, 1 H) | Intermediate 25 and 3,5- dimethoxy aniline |

### Intermediate 60: 5-Bromo-2-chloro-4-methoxyprimidine

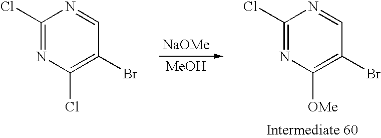

To a stirred solution of 5-bromo-2,4-dichloropyrimidine (65 mmol, 15 g) in methanol (150 mL) at room temperature under nitrogen atmosphere, sodium methoxide (72 mmol, 3.91 g) was added portionwise. The mixture was stirred at RT for 4h and the solvent was removed in vacuo. The resulting solid was dissolved in chloroform (2×250 mL), washed with water and brine. The organic layer was dried over sodium sulfate and concentrated under vacuum to yield 5-bromo-2-chloro-4-methoxypyrimidine as a white solid in 92% yield (60.4 mmol, 13.5 g). The compound was taken to the next step without further purification.

MS(ES): 223.0(M) and 224.8(M+2) for C₅H₄BrClN₂O.

¹H-NMR (300 MHz, CDCl₃): δ 4.11 (s, 3H), 8.44 (s, 1H)

### Intermediate 61: (5-bromo-4-methoxy-pyrimidin-2-yl)-(3-chloro-4-fluoro-phenyl)-amine

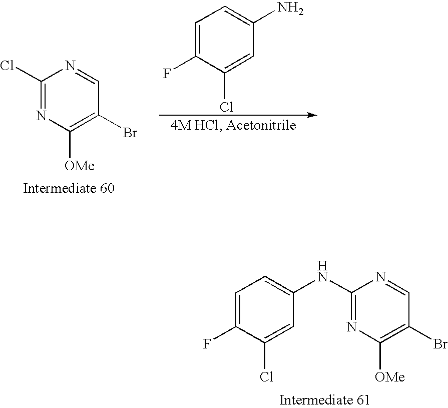

To a stirred solution of 5-bromo-2-chloro-4-methoxypyrimidine (Intermediate 60, 60 mmol, 13.5 g) and 3-chloro-4-fluoroaniline (60.3 mmol, 9.23 g) in acetonitrile (140 mL), 4 M HCl in 1,4-dioxane (14 mL) was added dropwise. The resulting solution was refluxed at 100° C. with constant stirring. The solvent was removed in vacuo and the residue was basified with 10% NaHCO₃ solution, then extracted with ethyl acetate (2×250 mL). The combined organic layers were washed with water and brine, dried over sodium sulfate and concentrated under vacuum. The residue was purified by column chromatography (silicagel, 60-120 mesh) using 5% EtOAc in hexane to yield (5-bromo-4-methoxy-pyrimidin-2-yl)-(3-chloro-4-fluoro-phenyl)-amine as a white solid in 70% yield (42 mmol, 14 g).

MS(ES): 332 (M) for C₁₁H₈BrClFN₃O.

¹H-NMR (400 MHz, DMSO-d6): δ 4.0 (s, 3H), 7.37 (t, J=9.20 Hz, 1H), 7.62 (ddd, J=9.02, 4.12, 2.84 Hz, 1H), 8.04 (dd, J=6.80, 2.60 Hz, 1H), 8.41 (s, 1H), 9.94 (s, 1H).

### Intermediate 62: 5-Bromo-2-(3-chloro-4-fluoroanilino)-pyrimidin-4-one

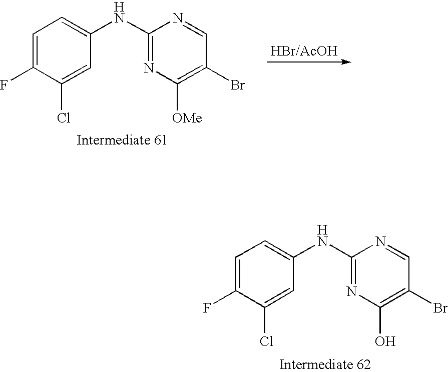

(5-Bromo-4-methoxy-pyrimidin-2-yl)-(3-chloro-4-fluoro-phenyl)-amine (Intermediate 61, 12 mmol, 4 g) was dissolved in 30-33% HBr in acetic acid (AcOH, 60 mmol, 13.5 g; 40 mL) and cooled to 0-5° C. 47% aqueous HBr (20 mL) was added and refluxed for 4 h. The reaction mass was cooled to RT and poured over crushed ice. After all of the ice melted, the product was collected by filtration and dried under vacuum to provide 5-bromo-2-(3-chloro-4-fluoroanilino)-pyrimidin-4-one (11.67 mmol, 3.7 g, 97% yield).

MS(ES): 318 (M) and 320 (M+2) for C₁₀H₆BrClFN₃O.

¹H-NMR (400 MHz, DMSO-d6): δ 7.34-7.43 (m, 1H), 7.44-7.45 (m, 1H), 7.91 (dd, J=6.74, 2.56 Hz, 1H), 8.08 (s, 1H), 9.11 (br s, 1H).

### Intermediate 63: 5-Bromo-2-(3-chloro-4-fluoroanilino)-4-chloro-pyrimidine

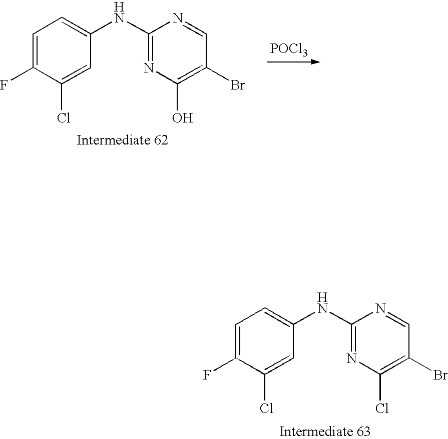

A solution of 5-bromo-2-(3-chloro-4-fluoroanilino)-pyrimidin-4-one (Intermediate 62, 18.3 mmol, 6.5 g) in phosphorus oxychloride (21 mL) was heated to reflux for 2 hours, cooled to RT, poured carefully onto a mixture of ice (200 mL) and saturated NaHCO₃ (20 mL) with stirring. The product was extracted with EtOAc (2×250 mL). The combined organic layers were washed with water, brine, dried over sodium sulfate, filtered and concentrated under vacuum. The residue was purified by column chromatography (silicagel, 60-120 mesh) using 3% EtOAc in hexane to yield 5-bromo-2-(3-chloro-4-fluoroanilino)-4-chloro-pyrimidine (15 mmol, 5.1 g).

MS(ES): 318 (M) and 320 (M+2) for C₁₀H₅BrCl₂FN₃

¹H NMR (400 MHz, DMSO-d6): δ 7.37 (t, J=9.12 Hz, 1H), 7.57 (ddd, J=8.90, 4.19, 2.54 Hz, 1H), 7.93 (dd, J=6.69, 2.54 Hz, 1H), 8.71 (s, 1H), 10.38 (s, 1H).

¹⁹F NMR (376 MHz, DMSO-d6): δ-124.12 (s, 1F).

### Intermediate 64: 5-bromo-N²-(3-chloro-4-fluorophenyl)-N⁴-[2-(1H-imidazol-1-yl)ethyl]pyrimidine-2,4-diamine

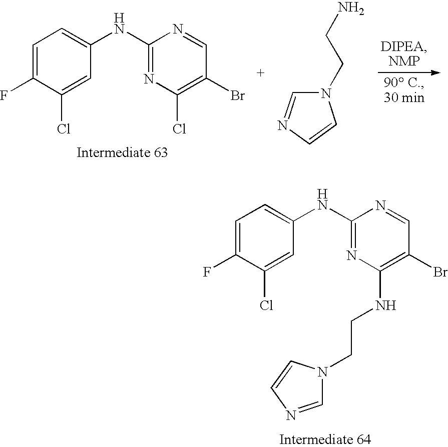

To 500 mg of 5-bromo-4-chloro-N-(3-chloro-4-fluorophenyl)pyrimidin-2-amine (Intermediate 63, 1.48 mmol) in NMP (5 mL), was added N,N-diisopropylethylamine (3.04 mL, 1.78 mmol) and 2-imidazol-1-yl-ethylamine (1.63 mmol, 181 mg) under inert atmosphere. The reaction mixture was heated to 90° C. for 30 min After completion of the reaction, the reaction mixture was quenched with water and extracted with ethyl acetate. The organic layer was washed with brine and dried over anhydrous Na₂SO₄. The crude mixture was purified by column chromatography using 2% MeOH in CHCl₃ to obtain 5-bromo-N²-(3-chloro-4-fluorophenyl)-N⁴-[2-(1H-imidazol-1-yl)ethyl]pyrimidine-2,4-diamine in 26% yield (0.39 mmol, 160 mg). MS(ES): 411 (M) and 413 (M+2) for C₁₅H₁₃BrClFN₆.

¹H-NMR (400 MHz, DMSO-d₆): δ 3.71 (q, J=6.00 Hz, 2H), 4.22 (t, J=6.20 Hz, 2H), 6.86 (s, 1H), 7.11 (s, 1H), 7.17 (t, J=5.64 Hz, 1H), 7.29 (t, J=9.12 Hz, 1H), 7.53-7.56 (m, 1H), 7.56 (s, 1H), 8.02 (dd, J=2.64, 6.84 Hz, 1H), 8.07 (s, 1H), 9.45 (s, 1H).

### Intermediate 65: (5-Bromo-4-methylsulfanyl-pyrimidin-2-yl)-(3-chloro-4-fluoro-phenyl)-amine

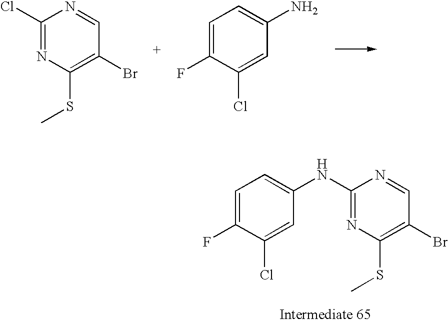

5-Bromo-2-chloro-4-(methylthio)pyrimidine (125 mmol, 30 g) was suspended in n-BuOH (300 mL) with 3-chloro-4-fluoroaniline (125 mmol, 18.22 g). The reaction was then treated with 4 N HCl (100 mmol, 25 mL) in dioxane and refluxed at 100° C. for 1.5 h under nitrogen. The reaction was cooled to room temperature, diluted with diethyl ether and the solid was filtered and dried to afford 29 g of (5-bromo-4-methylsulfanyl-pyrimidin-2-yl)-(3-chloro-4-fluoro-phenyl)-amine as a pale yellow solid (83 mmol, 67%).

MS(ES): 348(M) and 350(M+2) for C₁₁H₈BrClFN₃S.

¹H-NMR 300 MHz DMSO-d₆: δ 2.55 (s, 3H), 7.34 (t, J=9.0 Hz, 1H), 7.56-7.59 (m, 1H), 8.08 (t, J=4.5 Hz, 1H), 8.33 (s, 1H), 9.98 (s, 1H).

The following intermediates were prepared using the general method described above for Intermediate 65 using 5-bromo-2-chloro-4-(methylthio)pyrimidine and the starting material (SM) indicated.

| Int | Compound | Data | SM |
|---|---|---|---|
| 66 | 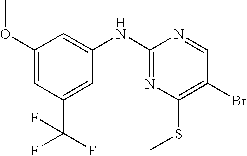 5-bromo-N-[3-methoxy-5- (trifluoromethyl)phenyl]- 4-(methylthio)pyrimidin- 2-amine | MS: ES+ 395 for C₁₃H₁₁BrF₃N₃OS 1H NMR (300 MHz, DMSO- d₆) δ ppm 2.56 (s, 3H) 3.79 (s, 3H) 6.82 (s, 1H) 7.53 (s, 1H) 7.87 (s, 1H) 8.36 (s, 1H) 10.10 (s, 1H) | 3-methoxy-5- trifluoro methylaniline |
| 67 | 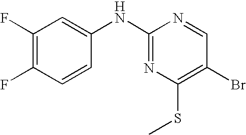 5-bromo-N-(3,4-difluorophenyl)-4- (methylthio)pyrimidin-2-amine | MS: ES+ 333 for C₁₁H₈BrF₂N₃S 1H NMR (300 MHz, DMSO- d₆) δ ppm 2.55 (s, 3H) 7.23- 7.49 (m, 2H) 7.90 (ddd, J = 13.85, 7.44, 2.45 Hz, 1H) 8.33 (s, 1H) 9.99 (s, 1H) | 3,4-difluoro aniline |
| 68 | 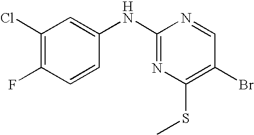 5-bromo-N-(3-chloro-4-fluorophenyl)- 4-(methylthio)pyrimidin-2- amine | MS: ES+ 349 for C₁₁H₈BrClFN₃S 1H NMR (300 MHz, DMSO- d₆) δ ppm 2.55 (s, 3H) 7.34 (t, J = 9.14 Hz, 1H) 7.57 (ddd, J = 9.09, 4.19, 2.73 Hz, 1H) 8.07 (dd, J = 6.78, 2.64 Hz, 1H) 8.33 (s, 1H) 9.96 (s, 1H) | 3-chloro-4- fluoro aniline |

### Intermediate 69: (5-Bromo-4-methanesulfonyl-pyrimidin-2-yl)-(3-chloro-4-fluoro-phenyl)-amine

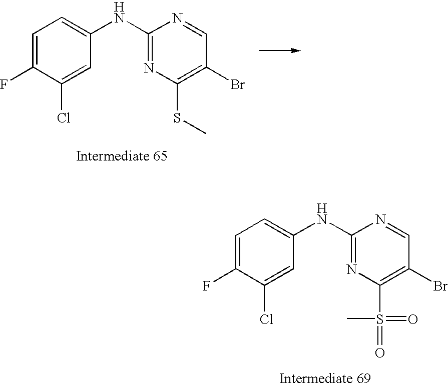

5-Bromo-2-[N-(3-chloro-4-fluorophenyl)]-4-(methylthio)pyrimidin-2-amine (Intermediate 65, 86 mmol, 30 g) was suspended in DCM (450 mL), cooled to 0° C. and 3-chloroperoxybenzoic acid (m-CPBA, 344 mmol, 59 g) was added. The suspension became a solution after stirring at 0° C. for 30 min. The reaction mixture was then allowed to warm up slowly to room temperature. 1N aqueous sodium hydroxide solution (344 mL) was added and the solution was stirred for 10 minutes. The solid which precipitated was filtered off and washed with water, dichloromethane, diethyl ether and dried. The first crop of the title compound, thus obtained, weighed 10 g. The mother liquor was dried over sodium sulfate and concentrated to yield a residue. Diethyl ether was added to the residue and the mixture was stirred for 15 minutes. The ether layer was filtered off. The residual solid was dissolved in CHCl₃-MeOH, then hexane was added to this mixture. The solid that precipitated was filtered off and dried. The second crop of the title compound, thus obtained, weighed 3.5 g. The total yield of (5-bromo-4-methanesulfonyl-pyrimidin-2-yl)-(3-chloro-4-fluoro-phenyl)-amine was 41% (35.3 mmol, 13.5 g). MS(ES): 380 (M) for C₁₁H₈BrClFN₃O₂S.

¹H-NMR 400 MHz DMSO-d₆: δ 3.45 (s, 3H), 7.40 (t, J=9.08 Hz, 1H), 7.55-7.58 (m, 1H), 7.96 (dd, J=6.74, 2.60 Hz, 1H), 8.93 (s, 1H), 10.51 (s, 1H).

The following intermediates were prepared using the general method described above for Intermediate 69 using m-CPBA and the starting material (SM) indicated.

| Int | Compound | Data | SM |
|---|---|---|---|
| 70 | 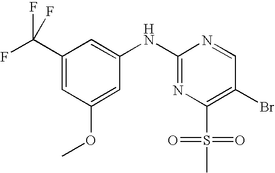 5-Bromo-N-(3-methoxy-5- (trifluoromethyl)phenyl)-4- (methylsulfonyl)pyrimidin- 2-amine | MS: ES+ 427 for C₁₃H₁₁BrF₃N₃O₃S 1H NMR (300 MHz, DMSO-d₆) δ ppm 3.46 (s, 3H) 3.82 (s, 3H) 6.91 (s, 1H) 7.55-7.60 (m, 1H) 7.70 (s, 1H) 8.97 (s, 1H) 10.63 (s, 1H) | Intermediate 66 |
| 71 | 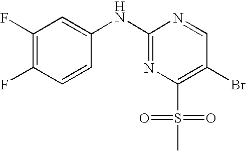 5-Bromo-N-(3,4-difluorophenyl)-4- (methylsulfonyl)pyrimidin-2-amine | MS: ES+ 365 for C₁₁H₈BrF₂N₃O₂S 1H NMR (300 MHz, DMSO-d₆) δ ppm 3.45 (s, 3H) 7.36-7.44 (m, 2H) 7.75- 7.89 (m, 1H) 8.93 (s, 1H) 10.53 (s, 1H) | Intermediate 67 |

### Intermediate 72: 1-{4-[5-Bromo-2-(3-chloro-4-fluoro-phenylamino)-pyrimidin-4-ylamino]-piperidin-1-yl}-ethanone

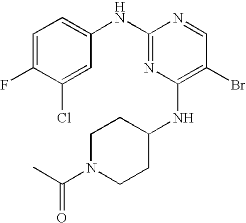

5- Bromo-2-[N-(3-chloro-4-fluorophenyl)]-4-(methyl sulfonyl)pyrimidin-2-amine (Intermediate 69, 1 g, 2.63 mmol) was suspended in NMP (5 mL), treated with N,N-diisopropylethylamine (0.5 mL, 3.02 mmol), and 1-(4-amino-piperidin-1-yl)-ethanone (1 eq 2.63 mmol) in a sealed tube. The reaction was heated in the microwave reactor at 100° C. for 30 min. The reaction mixture was added to water and stirred for 15 min. The precipitated solid was filtered and dried to provide the 1-{4-[5-bromo-2-(3-chloro-4-fluoro-phenylamino)-pyrimidin-4-ylamino]-piperidin-1-yl}-ethanone (800 mg).

MS(ES): 442.7 (M) and 444 (M+2) for C₁₇H₁₈BrClFN₅O.

400 MHz, DMSO-d₆: δ1.4-1.75 (m, 2H), 1.83-1.88 (m, 2H), 2.02 (s, 3H), 2.6-2.63 (m, 1H), 3.11 (t, J=12.00 Hz, 1H), 3.87-3.9 (m, 1H), 4.15-4.19 (m, 1H), 4.42-4.45 (m, 1H), 6.71 (d, J=8.00 Hz, 1H), 7.32 (t, J=9.04 Hz, 1H), 7.49-7.53 (m, 1H), 8.06 (s, 1H), 8.11 (dd, J=6.86, 2.60 Hz, 1H), 9.50 (s, 1H).

The following intermediates were prepared using the general method described above for Intermediate 72 using 5- bromo-2-[N-(3-chloro-4-fluorophenyl)]-4-(methyl sulfonyl)pyrimidin-2-amine (Intermediate 69) and the starting material (SM) indicated.

| Int | Compound | Data | SM |
|---|---|---|---|
| 73 | 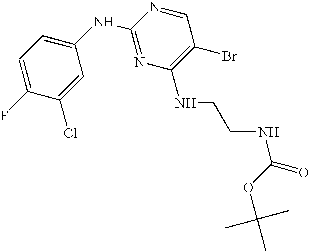 {2-[5-Bromo-2-(3-chloro-4-fluoro- phenylamino)-pyrimidin-4- ylamino]-ethyl}-carbamic acid tert- butyl ester | MS(ES): 461 (M + 1) and 462 (M + 2) for C₁₇H₂₀BrClFN₅O₂. 400 MHz, DMSO-d₆: δ 1.34 (s, 9H), 3.17-3.2 (m, 2H), 3.41-3.44 (m, 2H), 6.91 (t, J = 5.04 Hz, 1H), 7.01 (t, J = 5.00 Hz, 1H), 7.28 (t, J = 8.96 Hz, 1H), 7.60 (s, 1H), 8-8.03 (m, 2H), 9.43 (s, 1H). | (2-Amino- ethyl)- carbamic acid tert-butyl ester |
| 74 | 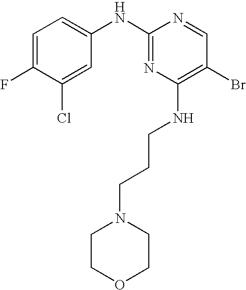 5-Bromo-N²-(3-chloro-4-fluoro- phenyl)-N⁴-(3-morpholin-4-yl- propyl)-pyrimidine-2,4-diamine | MS (ES): 444 (M) and 446 (M + 2) for C₁₇H₂₀BrClFN₅O. 400 MHz, DMSO-d₆: δ 1.72-1.77 (m, 2H) 2.33-2.38 (m, 6H), 3.42- 3.46 (m, 2H), 3.55-3.57 (t, J = 9.04 Hz, 4H), 7.22-7.30 (m, 2H), 7.53 (ddd, J = 8.98, 4.16, 2.76 Hz, 1H), 8.02 (s, 1H), 8.13 (dd, J = 6.88, 2.60 Hz, 1H), 9.43 (s, 1H). | 3-Morpholin- 4-yl- propylamine |
| 75 | 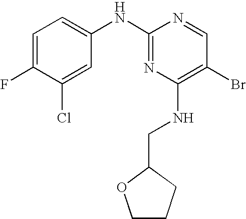 5-Bromo-N2-(3-chloro-4-fluoro- phenyl)-N4-(tetrahydrofuran-2- ylmethyl)-pyrimidine-2,4- diamine | MS(ES): 401 (M) for C₁₅H₁₅BrClFN₄O. 400 MHz DMSO-d₆: δ 1.56 (m, 1H), 1.78 (m, 3H), 2.49 (t, J = 1.56 Hz, 2H), 3.43-3.63 (m, 1H), 3.73 (q, J = 6.64 Hz, 1H), 4.1 (m, 1H), 7.38 (t, J = 9 Hz, 1H), 7.47 (m, 1H), 7.95 (q, J = 2.48, 6.76 Hz, 1H), 8.15 (br s, 1H), 8.23 (s, 1H), 10.5 (br s, 1H). | C- (Tetrahydro- furan-2-yl)- methylamine |
| 76 | 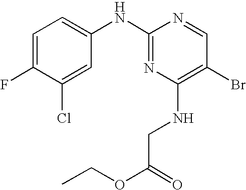 [5-Bromo-2-(3-chloro-4-fluoro- phenylamino)-pyrimidin-4- ylamino]-acetic acid ethyl ester | The compound was taken to the next step on the basis of ¹H NMR. 400 MHz, DMSO-d₆ δ: 1.13 (t, J = 7.08 Hz, 3H), 4.06 (q, J = 7.12 Hz, 2H), 4.11 (d, J = 6.00 Hz, 2H), 7.25 (t, J = 9.08 Hz, 1H), 7.44 (t, J = 6.00 Hz, 1H), 7.50 (dt, J = 8.57, 4.08 Hz, 1H), 7.94 (dd, J = 6.80, 2.56 Hz, 1H), 8.10 (s, 1H), 9.47 (s, 1H). | Amino-acetic acid ethyl ester |
| 77 | 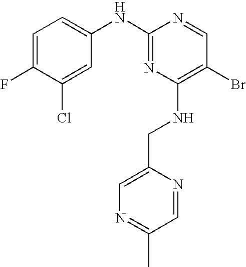 5-Bromo-N2-(3-chloro-4-fluoro- phenyl)-N4-(5-methyl-pyrazin-2- ylmethyl)-pyrimidine-2,4- diamine | MS(ES): 423 (M + 2) for C₁₆H₁₃BrClFN₆ The compound was taken to the next step on the basis of mass spectrum. | C-(5-Methyl- pyrazin-2- yl)- methylamine |
| 78 | 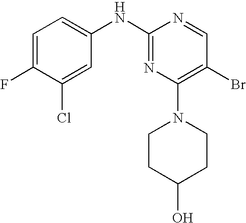 1-[5-Bromo-2-(3-chloro-4-fluoro- phenylamino)-pyrimidin-4-yl]- piperidin-4-ol | MS(ES): 401 (M) and 402 (M + 1) for C₁₅H₁₅BrClFN₄O. 400 MHz, DMSO-d₆: δ 1.6 (m, 2H), 1.8 (m, 2H), 3.2 (m, 2H), 3.8 (m, 1H), 3.9 (m, 2H), 7.31 (t, 1H), 7.5 (m, 1H), 8.1 (d, 1H), 8.2 (s, 1H). | Piperidin-4- ol |
| 79 | 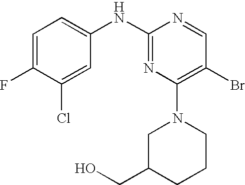 {1-[5-Bromo-2-(3-chloro-4-fluoro- phenylamino)-pyrimidin-4-yl]- piperidine-3-yl}-methanol | MS(ES): 415 (M) and 417 (M + 2) for C₁₆H₁₇BrClFN₄O. 300 MHz, DMSO-d₆ δ: 1.2 (m, 1H), 1.5-1.7 (m, 3H), 2.73 (t, J = 10.47 Hz, 1H), 2.92 (t, J = 10.53 Hz, 1H), 4.12 (d, J = 13.02 Hz, 1H), 4.24 (d, J = 13.11 Hz, 1H), 7.29 (t, J = 9.12 Hz, 1H), 7.55-7.59 (m, 1H), 8.04 (dd, J = 2.58, 6.84 Hz, 1H), 8.19 (s, 1H), 9.61 (s, 1H). | Piperidin-3- yl-methanol |
| 80 | 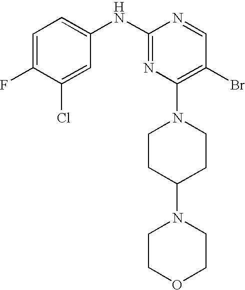 [5-Bromo-4-(4-morpholin-4-yl- piperidin-1-yl)-pyrimidin-2-yl]-(3- chloro-4-fluoro-phenyl)-amine | MS(ES): 471 (M + 1) and 472 (M + 2) for C₁₉H₂₂BrClFN₅O. 300 MHz CDCl₃: δ 2.0 (br s, 2H), 2.6 (br s, 4H), 2.94 (br s, 2H), 3.74 (br s, 4H), 4.41-4.45 (br s, 2H), 6.86 (s, 1H), 7.1 (t, J = 8.7 Hz, 1H), 7.17 (m, 1H), 7.95 (dd, J = 3, 6.6 Hz, 1H), 8.12 (s, 1H). | 4-Piperidin- 4-yl- morpholine |
| 81 | 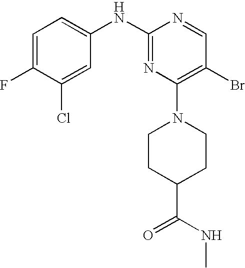 1-[5-Bromo-2-(3-chloro-4-fluoro- phenylamino)-pyrimidin-4-yl]- piperidine-4-carboxylic acid methylamide | MS(ES): 441.9 (M) and 444 (M + 2) for C₁₇H₁₈BrClFN₅O. 400 MHz, DMSO-d₆: δ 1.64 (m, 2H), 1.77 (m, 2H), 2.38 (m, 1H), 2.55 (d, J = 4.52 Hz, 3H), 2.95 (t, J = 11.6 Hz, 2H), 4.26 (d, J = 13.04 Hz, 2H), 7.30 (t, J = 9.12 Hz, 1H), 7.52 (m, 1H), 7.77 (d, J = 4.6 Hz, 1H), 8.08 (dd, J = 2.48, 6.84 Hz, 1H), 8.21 (s, 1H), 9.64 (s, 1H). | Piperidine-4- carboxylic acid methylamide |
| 82 | 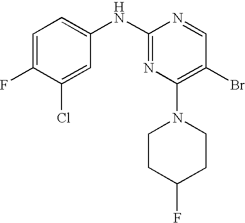 [5-Bromo-4-(4-fluoro-piperidin-1- yl)-pyrimidin-2-yl]-(3-chloro-4-fluoro- phenyl)-amine | MS(ES): 403 (M) for C₁₅H₁₄BrClF₂N₄. 300 MHz, DMSO-d₆: δ 1.82 (m, 2H), 1.94-2.04 (m, 2H), 3.56-3.68 (m, 4H), 4.92 (d, J = 48.6 Hz, 1H), 7.31 (t, J = 9.12 Hz, 1H), 7.51-7.56 (m, 1H), 8.06 (dd, J = 6.81, 2.49 Hz, 1H), 8.24 (s, 1H), 9.67 (br s, 1H). | 4-Fluoro- piperidine |
| 83 | 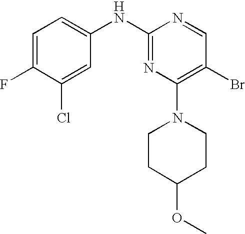 [5-Bromo-4-(4-methoxy-piperidin- 1-yl)-pyrimidin-2-yl]-(3-chloro-4- fluoro-phenyl)-amine | MS(ES): 415 (M) and 417 (M + 2) for C₁₆H₁₇BrClFN₄O. 400 MHz, DMSO-d₆: δ 1.48-1.57 (m, 2H), 1.88-1.96 (m, 2H), 3.24- 3.30 (m, 4H), 3.42-3.46 (m, 1H), 3.87-3.90 (m, 2H), 7.3 (t, J = 9.12 Hz, 1H), 7.50-7.54 (m, 1H), 8.08 (dd, J = 6.86, 2.56 Hz, 1H), 8.21 (s, 1H), 9.64 (s, 1H). | 4-methoxy- piperidine |
| 84 | 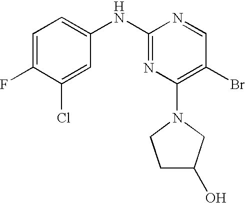 1-[5-Bromo-2-(3-chloro-4-fluoro- phenylamino)-pyrimidin-4-yl]- pyrrolidin-3-ol | MS(ES): 387 (M) and 389 (M + 2) for C₁₄H₁₃BrClFN₄O. 400 MHz, DMSO-d₆: δ 1.87-1.95 (m, 2H), 3.65 (d, J = 11.44 Hz, 1H), 3.78-3.85 (m, 3H), 4.35 (s, 1H), 5.03 (d, J = 3.40 Hz, 1H), 7.30 (t, J = 9.12 Hz, 1H), 7.56 (ddd, J = 9.06, 4.24, 2.72 Hz, 1H), 8.08 (s, 1H), 8.11 (dd, J = 6.90, 2.68 Hz, 1H), 9.48 (s, 1H). | Pyrrolidin-3- ol |
| 85 | 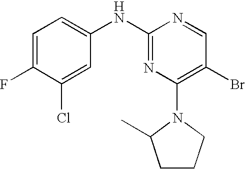 [5-Bromo-4-(2-methyl-pyrrolidin- 1-yl)-pyrimidin-2-yl]-(3-chloro-4- fluoro-phenyl)-amine | MS(ES): 385 (M) and 387 (M + 2) for C₁₅H₁₅BrClFN₄. 300 MHz DMSO-d₆: δ 1.2 (d, 3H), 1.6 (m, 1H), 1.8 (m, 1H), 1.9-2.1 (m, 2H), 3.68 (m, 1H), 3.87-3.96 (m, 1H), 4.55 (m, 1H), 7.29 (t, J = 9.09 Hz, 1H), 7.50 (dt, J = 2.76, 9.12 Hz, 1H), 8.09-8.12 (m, 2H), 9.5 (s, 1H). | 2-Methyl- pyrrolidine |
| 86 | 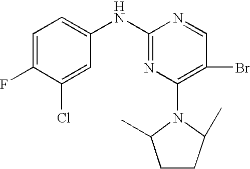 [5-Bromo-4-(2,5-dimethyl- pyrrolidin-1-yl)-pyrimidin-2-yl]-(3- chloro-4-fluoro-phenyl)-amine | MS(ES): 399 (M) and 401 (M + 2) for C₁₆H₁₇BrClFN₄. 400 MHz DMSO-d₆: δ 1.30 (d, 6H), 1.74 (m, 2H), 2.03 (m, 2H), 4.62 (br s, 2H), 7.29 (t, J = 9.08 Hz, 1H), 7.46 (ddd, J = 8.98, 4.12, 2.80 Hz, 1H), 8.08-8.11 (m, 2H), 9.43 (s, 1H). | 2,5- Dimethyl- pyrrolidine |
| 87 | 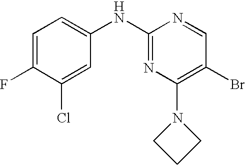 (4-Azetidin-1-yl-5-bromo-pyrimidin-2- yl)-(3-chloro-4-fluoro-phenyl)- amine | MS(ES): 356.9 (M) and 359 (M + 2) for C₁₃H₁₁BrClFN₄ 400 MHz, DMSO-d₆: δ: 2.32 (q, J = 7.64 Hz, 2H), 4.40 (s, 4H), 7.34 (t, J = 9.12 Hz, 1H), 7.52 (ddd, J = 9.04, 4.24, 2.68 Hz, 1H), 8.04 (dd, J = 6.84, 2.60 Hz, 1H), 8.10 (s, 1H), 9.96 (s, 1H). | Azetidine |
| 88 | 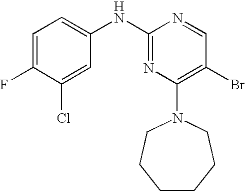 (4-Azepan-1-yl-5-bromo-pyrimidin-2- yl)-(3-chloro-4-fluoro-phenyl)- amine | MS(ES): 399 (M) and 401 (M + 2) for C₁₆H₁₇BrClFN₄. 400 MHz, DMSO-d₆: δ 1.51-1.52 (m, 4H), 1.79 (m, 4H), 3.83 (t, J = 6.04 Hz, 4H), 7.30 (t, J = 9.08 Hz, 1H), 7.47 (ddd, J = 9.09, 4.24, 2.72 Hz, 1H), 8.08 (dd, J = 6.90, 2.68 Hz, 1H), 8.10 (s, 1H), 9.48 (s, 1H). | Azepane |
| 89 | 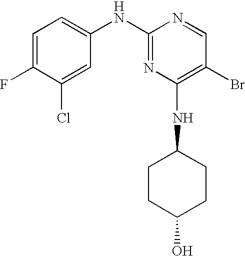 Trans-4-[5-Bromo-2-(3-chloro-4- fluoro-phenylamino)- pyrimidin-4-ylamino]- cyclohexanol | MS(ES): 415 (M) and 417 (M + 2) for C₁₆H₁₇BrClFN₄O. 300 MHz, DMSO-d₆: δ 1.27-1.34 (m, 2H), 1.42-1.49 (m, 2H), 1.83- 1.86 (br s, 4H), 3.89 (br s, 1H), 4.57 (d, J = 4.35 Hz, 1H), 6.48 (d, J = 8.07 Hz, 1H), 7.28 (t, J = 8.94 Hz, 1H), 7.48 (m, 1H), 8.03 (s, 1H), 8.14 (dd, J = 2.43, 6.9 Hz, 1H), 9.45 (s, 1H). | 4-Amino- cyclohexanol |
| 90 | 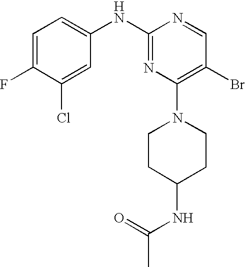 N-{1-[5-Bromo-2-(3-chloro-4-fluoro- phenylamino)-pyrimidin-4-yl]- piperidin-4-yl}-acetamide | MS(ES): 442 (M) and 444 (M + 2) for C₁₇H₁₈BrClFN₅O. 400 MHz, DMSO-d₆ δ: 1.49 (d, J = 7.28 Hz, 3H), 3.60 (s, 3H), 4.69 (m, 1H), 7.11 (d, J = 7.36 Hz, 1H), 7.27 (t, J = 9.08 Hz, 1H), 7.52 (ddd, J = 9.06, 4.18, 2.72 Hz, 1H), 7.93 (dd, J = 6.80, 2.56 Hz, 1H), 8.12 (s, 1H), 9.47 (s, 1H). | N-Piperidin- 4-yl- acetamide |
| 91 | 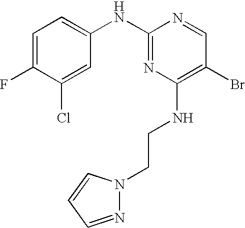 5-bromo-N²-(3-chloro-4- fluorophenyl)-N⁴-[2-(1H-pyrazol-1- yl)ethyl]pyrimidine-2,4-diamine | MS(ES): 411 (M) and 413 (M + 2) for C₁₅H₁₃BrClFN₆. 400 MHz, DMSO-d₆: δ 3.78 (q, J = 6.12 Hz, 2H), 4.36 (t, J = 6.40 Hz, 2H), 6.23 (t, J = 1.92 Hz, 1H), 7.13 (t, J = 5.52 Hz, 1H), 7.29 (t, J = 9.12 Hz, 1H), 7.46 (d, J = 1.68 Hz, 1H), 7.63-7.67 (m, 2H), 8.00 (dd, J = 2.28, 6.90 Hz, 1H), 8.06 (d, J = 2.12 Hz, 1H), 9.47 (s, 1H). | 2-(1H- pyrazol-1- yl)ethanamine |
| 92 | 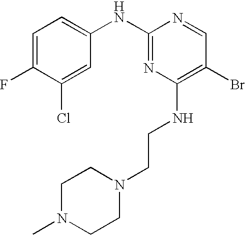 5-bromo-N²-(3-chloro-4- fluorophenyl)-N⁴-[2-(4-methylpiperazin-1- yl)ethyl]pyrimidine-2,4-diamine | MS(ES): 443 (M) and 445 (M + 2) for C₁₇H₂₁BrClFN₆. 400 MHz, DMSO-d₆: δ 2.30 (br s, 4H), 2.44 (br s, 4H), 2.69 (s, 3H), 3.30 (t, J = 6.92 Hz, 2H), 3.50 (q, J = 6.16 Hz, 2H), 6.89 (t, J = 5.20 Hz, 1H), 7.27 (t, J = 9.08 Hz, 1H), 7.58 (ddd, J = 2.52, 4.26, 9.02 Hz, 1H), 8.04-8.07 (m, 2H), 9.44 (s, 1H). | 2-(4- methylpiper- azin-1- yl)ethanamine |
| 93 | 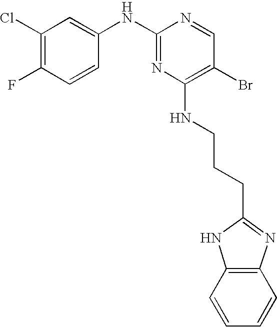 N⁴-(3-(1H-benzo[d]imidazol- 2-yl)propyl)-5-bromo-N²-(3-chloro-4- fluorophenyl)pyrimidine-2,4-diamine | MS(ES): 475 (M) 477 (M + 2) for C₂₀H₁₇BrClFN₆ 1H NMR (300 MHz, DMSO-d6) δ ppm 2.02-2.22 (m, 2H) 2.89 (t, J = 7.54 Hz, 2H) 3.52 (q, J = 6.22 Hz, 2H) 6.98-7.15 (m, 2H) 7.24 (t, J = 9.14 Hz, 1H) 7.29-7.46 (m, 2H) 7.44-7.72 (m, 2H) 8.04 (s, 1H) 8.11 (dd, J = 6.78, 2.64 Hz, 1H) 9.46 (s, 1H) 12.19 (s, 1H) | 3-(1H- benzo[d]imidazol- 2-yl)propan-1- amine |
| 94 | 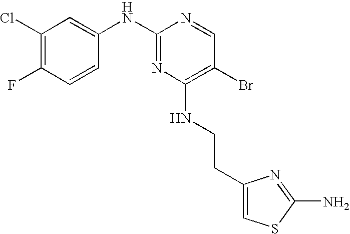 N⁴-(2-(2-aminothiazol-4-yl)ethyl)-5-bromo- N²-(3-chloro-4-fluorophenyl)pyrimidin-2,4-diamine | MS(ES): 445 (M + 2) for C₁₅H₁₃BrClFN₆S 1H NMR (300 MHz, DMSO-d6) d ppm 2.21 (s, 3H) 2.31-2.44 (m, 4H) 3.15-3.35 (m, 4H) 7.33 (t, J = 9.04 Hz, 1H) 7.61 (dd, J = 4.90, 3.20 Hz, 1H) 8.00-8.25 (m, 2H) 8.30 (s, 1H) 8.86 (d, J = 1.32 Hz, 1H) 8.98 (s, 1H) 9.72 (s, 1H) | 4-(2- aminoethyl)- thiazol-2- amine dihydrochloride |
| 95 | 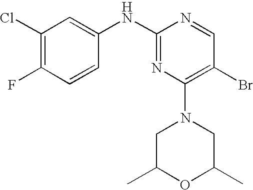 5-bromo-N-(3-chloro-4- fluorophenyl)-4-(2,6- dimethylmorpholino)-pyrimidin-2- amine | MS(ES): 415 (M) 417 (M + 2) for C₁₆H₁₇BrClFN₄O 1H NMR (300 MHz, DMSO-d₆) δ ppm 1.14 (d, 6H) 2.55-2.77 (m, 2H) 3.59-3.78 (m, 2H) 4.15 (d, J = 12.62 Hz, 2H) 7.32 (t, J = 9.04 Hz, 1H) 7.48 (ddd, J = 8.95, 4.14, 2.73 Hz, 1H) 8.10 (dd, J = 6.78, 2.64 Hz, 1H) 8.26 (s, 1H) 9.70 (s, 1H) | 2.6-dimethyl- morpholine |
| 96 | 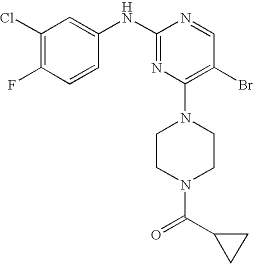 (4-(5-bromo-2-(3-chloro-4- fluorophenylamino)pyrimidin-4- yl)piperazin-1-yl)(cyclopropyl)- methanone | MS(ES): 454 (M) 456 (M + 2) for C₁₈H₁₈BrClFN₅O 1H NMR (300 MHz, DMSO-d₆) δ ppm 0.60-0.90 (m, 4H) 1.91- 2.11 (m, 1H) 3.45-3.98 (m, 8H) 7.33 (t, J = 9.14 Hz, 1H) 7.58 (ddd, J = 9.04, 4.14, 2.64 Hz, 1H) 8.03 (dd, J = 6.78, 2.64 Hz, 1H) 8.28 (s, 1H) 9.71 (s, 1H) | 1- (cyclopropyl- carbonyl)- piperazine |
| 97 | 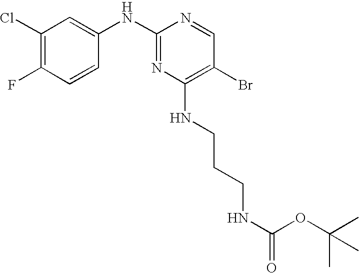 tert-Butyl 3-(5-bromo-2-(3-chloro-4- fluorophenylamino)pyrimidin-4- ylamino)propylcarbamate | MS(ES): 474 (M) 476 (M + 2) for C₁₈H₂₂BrClFN₅O₂ 1H NMR (300 MHz, DMSO-d₆) δ ppm 1.36 (s, 9H) 1.57-1.80 (m, 2H) 2.99 (q, J = 6.47 Hz, 2H) 3.35- 3.51 (m, 2H) 6.87 (t, J = 5.46 Hz, 1H) 7.11 (t, J = 5.93 Hz, 1H) 7.31 (t, J = 9.14 Hz, 1H) 7.59 (dd, J = 9.42, 3.58 Hz, 1H) 7.94-8.21 (m, 2H) 9.46 (s, 1H) | N-(3-Amino- propyl)car- bamic acid tert- butyl ester |
| 98 | 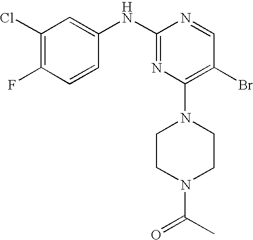 1-{4-[5-bromo-2-(3-chloro-4- fluorophenylamino)pyrimidin-4- yl]piperazin-1-yl}ethanone | MS(ES): 428 (M) 430 (M + 2) for C₁₆H₁₆BrClFN₅O 1H NMR (300 MHz, DMSO-d6) δ ppm 2.03 (s, 3H) 3.43-3.78 (m, 8H) 7.32 (t, J = 9.14 Hz, 1H) 7.46- 7.66 (m, 1H) 8.01 (dd, J = 6.88, 2.54 Hz, 1H) 8.27 (s, 1H) 9.70 (s, 1H) | 1-Acetyl- piperazine |
| 99 | 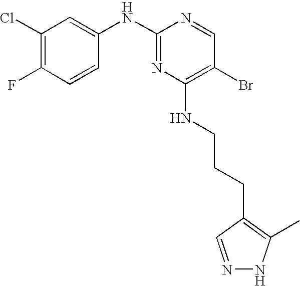 5-bromo-N²-(3-chloro-4- fluorophenyl)-N⁴-(3-(5-methyl-1H- pyrazol-4-yl)propyl)pyrimidine- 2,4-diamine | MS(ES): 441 (M + 2) for C₁₇H₁₇BrClFN₆ 1H NMR (300 MHz, DMSO-d₆) δ ppm 1.57-1.95 (m, 2H) 2.08 (br. s., 3H) 2.40 (t, J = 7.54 Hz, 2H) 3.36-3.53 (m, 2H) 6.96-7.73 (m, 4H) 7.87-8.30 (m, 2H) 9.44 (s, 1H) 11.94-12.38 (m, 1H) | 3-(5-Methyl- 1H-pyrazol- 4- yl)propyl- amine |
| 100 | 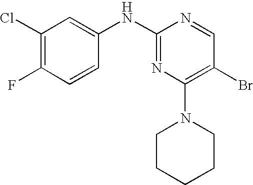 5-bromo-N-(3-chloro-4- fluorophenyl)-4-(piperidin-1- yl)pyrimidin-2-amine | MS(ES): 385 (M) 387 (M + 2) for C₁₅H₁₅BrClFN₄ 1H NMR (300 MHz, DMSO-d₆) δ ppm 1.63 (br. s., 6H) 3.57 (br. s., 4H) 7.32 (t, J = 9.04 Hz, 1H) 7.54 (ddd, J = 9.09, 4.19, 2.73 Hz, 1H) 8.11 (dd, J = 6.88, 2.54 Hz, 1H) 8.21 (s, 1H) 9.64 (s, 1H) | Piperidine |
| 101 | 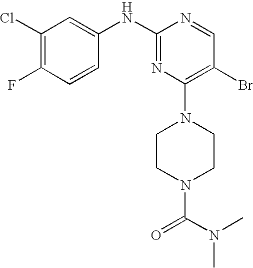 4-(5-bromo-2-(3-chloro-4- fluorophenylamino)pyrimidin-4-yl)- N,N-dimethylpiperazine- 1-carboxamide | MS(ES): 457 (M) 459 (M + 2) for C₁₇H₁₉BrClFN₆O 1H NMR (300 MHz, DMSO-d₆) δ ppm 2.77 (s, 6H) 3.12-3.30 (m, 4H) 3.51-3.73 (m, 4H) 7.33 (t, J = 9.14 Hz, 1H) 7.57 (ddd, J = 9.04, 4.14, 2.83 Hz, 1H) 8.04 (dd, J = 6.88, 2.54 Hz, 1H) 8.27 (s, 1H) 9.70 (s, 1H) | piperazine-1- carboxylic acid dimethylamide |
| 102 | 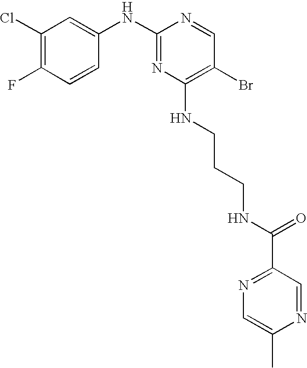 N-(3-(5-bromo-2-(3-chloro-4- fluorophenylamino)pyrimidin-4- ylamino)propyl)-5-methylpyrazine-2- carboxamide | MS(ES): 494 (M) 496 (M + 2) for C₁₉H₁₈BrClFN₇O 1H NMR (300 MHz, DMSO-d₆) δ ppm 1.77-1.95 (m, 2H) 2.58 (s, 3H) 3.37 (q, J = 6.03 Hz, 2H) 3.47 (q, J = 5.97 Hz, 2H) 7.30 (t, J = 9.14 Hz, 1H) 7.43-7.66 (m, J = 4.52, 4.52, 4.24, 2.73 Hz, 2H) 7.96-8.20 (m, 2H) 8.59 (d, J = 0.94 Hz, 1H) 8.89- 9.13 (m, 2H) 9.65 (s, 1H) | 3-[(5-Methyl- pyrazine-2- carbonyl)amino] propylamine hydrochloride |
| 103 | 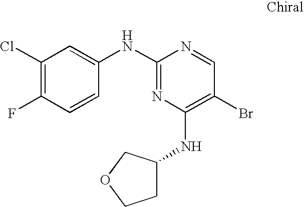 (R)-5-bromo-N²-(3-chloro-4- fluorophenyl)-N⁴-(tetrahydrofuran-3- yl)pyrimidine-2,4-diamine | MS(ES): 387 (M) 389 (M + 3) for C₁₄H₁₃BrClFN₄O 1H NMR (300 MHz, DMSO-d₆) δ ppm 1.90-2.13 (m, 1H) 2.13- 2.33 (m, 1H) 3.57-3.81 (m, 2H) 3.81-4.05 (m, 2H) 4.42-4.72 (m, 1H) 6.86 (d, J = 6.22 Hz, 1H) 7.31 (t, J = 9.14 Hz, 1H) 7.52(ddd, J = 9.14, 4.24, 2.64 Hz, 1H) 8.03- 8.21 (m, 2H) 9.51 (s, 1H) | R(+)-3- Aminotetra- hydrofuran |
| 104 | 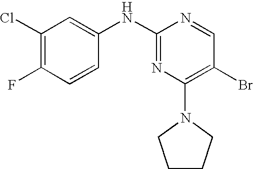 5-bromo-N-(3-chloro-4- fluorophenyl)-4-(pyrrolidin-1- yl)pyrimidin-2-amine | MS(ES): 371 (M) 373 (M + 2) for C₁₄H₁₃BrClFN₄ 1H NMR (300 MHz, DMSO-d₆) δ ppm 1.75-2.04 (m, 4H) 3.74 (t, J = 6.40 Hz, 4H) 7.30 (t, J = 9.14 Hz, 1H) 7.57 (ddd, J = 9.04, 4.24, 2.73 Hz, 1H) 7.99-8.21 (m, 2H) 9.48 (s, 1H) | Pyrrolidine |
| 105 | 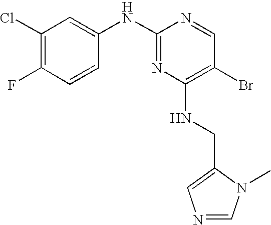 5-bromo-N²-(3-chloro-4- fluorophenyl)-N⁴-((1-methyl-1H- imidazol-5-yl)methyl)pyrimi- dine-2,4-diamine | MS(ES): 411 (M) 413 (M + 2) for C₁₅H₁₃BrClFN₆ 1H NMR (300 MHz, DMSO-d₆) δ ppm 3.62 (s, 3H) 4.57 (d, J = 5.65 Hz, 2H) 6.79 (s, 1H) 7.29 (t, J = 9.14 Hz, 1H) 7.40-7.67 (m, 3H) 8.02 (dd, J = 6.88, 2.54 Hz, 1H) 8.09 (s, 1H) 9.45 (s, 1H) | (1-Methyl- 1H-imidazol- 5-yl)methan- amine |
| 106 | 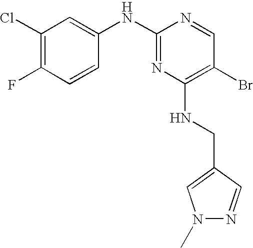 5-bromo-N²-(3-chloro-4- fluorophenyl)-N⁴-((1-methyl-1H- pyrazol-4-yl)methyl)pyrimi- dine-2,4-diamine | MS(ES): 411 (M) 413 (M + 2) for C₁₅H₁₃BrClFN₆ 1H NMR (300 MHz, DMSO-d₆) δ ppm 3.75 (s, 3H) 4.42 (d, J = 5.84 Hz, 2H) 7.30 (t, J = 9.14 Hz, 1H) 7.36 (s, 1H) 7.44 (t, J = 5.75 Hz, 1H) 7.50-7.66 (m, 2H) 7.97-8.19 (m, 2H) 9.45 (s, 1H) | (1-Methyl- 1H-pyrazol-4- yl)methan- amine |
| 107 | 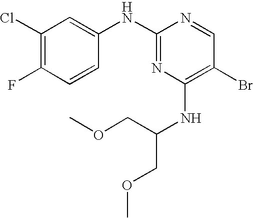 5-bromo-N²-(3-chloro-4- fluorophenyl)-N⁴-(1,3-dimethoxypropan- 2-yl)pyrimidine-2,4-diamine | MS(ES): 419 (M) 421 (M + 2) for C₁₅H₁₇BrClFN₄O₂ 1H NMR (300 MHz, DMSO-d₆) δ ppm 3.27 (s, 6H) 3.38-3.64 (m, 4H) 4.33-4.70 (m, 1H) 6.46 (d, J = 8.48 Hz, 1H) 7.28 (t, J = 9.14 Hz, 1H) 7.55 (ddd, J = 9.04, 4.24, 2.73 Hz, 1H) 7.96-8.17 (m, 2H) 9.48 (s, 1H) | 2-Amino-1,3- dimethoxy- propane |
| 108 | 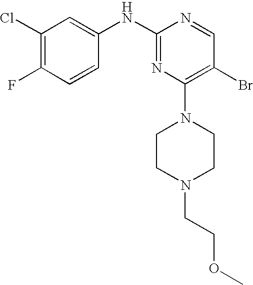 5-bromo-N-(3-chloro-4- fluorophenyl)-4-[4-(2-methoxyethyl)piperazine-1- yl]pyrimisin-2-amine | MS(ES): 444 (M) 446 (M + 2) for C₁₇H₂₀BrClFN₅O 1H NMR (300 MHz, DMSO-d₆) δ ppm 2.52-2.64 (m, 6H) 3.24 (s, 3H) 3.46 (t, J = 5.65 Hz, 2H) 3.58- 3.56 (m, 4H) 7.32 (t, J = 9.14 Hz, 1H) 7.55 (dt, J = 9.04, 3.39 Hz, 1H) 8.07 (dd, J = 6.88, 2.54 Hz, 1H) 8.24 (s, 1H) 9.68 (s, 1H) | 1-(2- Methoxyethyl) piperazine |
| 109 | 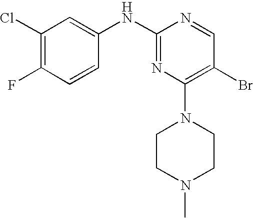 5-bromo-N-(3-chloro-4- fluorophenyl)-4-(4-methylpiperazin-1- yl)pyrimidin-2-amine | MS(ES): 400 (M) 402 (M + 2) for C₁₅H₁₆BrClFN₅ 1H NMR (300 MHz, DMSO-d₆) δ ppm 2.22 (s, 3H) 2.31-2.47 (m, 4H) 3.47-3.70 (m, 4H) 7.32 (t, J = 9.04 Hz, 1H) 7.54 (ddd, J = 9.04, 4.14, 2.64 Hz, 1H) 8.08 (dd, J = 6.78, 2.64 Hz, 1H) 8.24 (s, 1H) 9.69 (s, 1H) | 1-Methyl- piperazine |
| 110 | 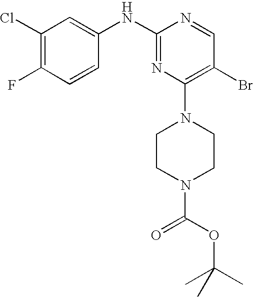 tert-butyl 4-[5-bromo-2-(3-chloro-4- fluorophenylamino)pyrimidin-4- yl]piperazine-1-carboxylate | MS(ES): 486 (M) 488 (M + 2) for C₁₉H₂₂BrClFN₅O₂ 1H NMR (300 MHz, DMSO-d6) δ ppm 1.41 (s, 9H) 3.36-3.72 (m, 8H) 7.33 (t, J = 9.14 Hz, 1H) 7.45- 7.70 (m, 1H) 8.02 (dd, J = 6.88, 2.54 Hz, 1H) 8.27 (s, 1H) 9.70 (s, 1H) | tert-butyl piperazine-1- carboxylate |

### Intermediate 111: 5-Bromo-N-(3-chloro-4-fluorophenyl)-4-morpholin-4-ylpyrimidin-2-amine

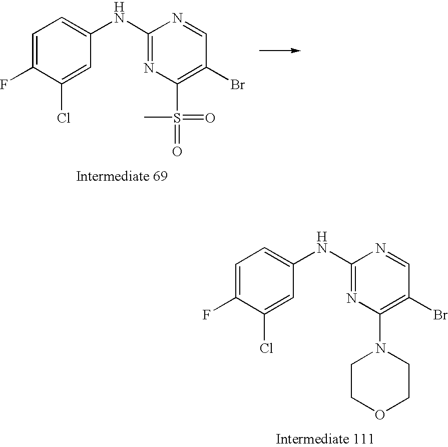

5-Bromo-2-[N-(3-chloro-4-fluorophenyl)]-4-(methylsulfonyl)pyrimidin-2-amine (Intermediate 69, 15.7 mmol, 6 g) was suspended in NMP (30 mL) and treated with N,N-diisopropylethylamine (23.6 mmol, 4 mL) and morpholine (18.9 mmol, 1.64 g) in a 100 mL round-bottomed flask. The reaction was heated to 90° C. for 45 min. The reaction mixture was added to water and stirred for 15 min The precipitated solid was filtered and washed successively with water, diethyl ether and hexanes and dried to afford 4.96 g of the title compound (12.56 mmol, 80%).

MS(ES): 387 (M) and 389 (M+2) for C₁₄H₁₃BrClFN₄O.

¹H NMR 400 MHz DMSO-d₆: δ 3.57-3.59 (m, 4H), 3.71-3.73 (m, 4H), 7.32 (t, J=9.08 Hz, 1H), 7.55 (ddd, J=9.00, 4.18, 2.68 Hz, 1H), 8.04 (dd, J=6.86, 2.64 Hz, 1H), 8.26 (d, J=1.20 Hz, 1H), 9.71 (s, 1H).

### Intermediate 112: 5-Bromo-N-(3-chloro-4-fluorophenyl)-4-(3,5-dimethyl-1H-pyrazol-1-yl)pyrimidin-2-amine

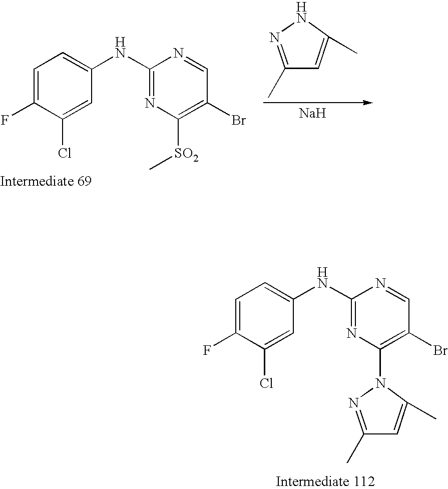

A solution of 3,5-dimethyl-1H-pyrazole (554 mg, 5.78 mmol) in DMF (1 ml) was added slowly to a suspension of sodium hydride (60%, 208 mg, 5.52 mmol) in DMF (1 ml) at 0° C. and the resultant mixture was stirred for 25 min. A solution of 5-bromo-N-(3-chloro-4-fluorophenyl)-4-(methylsulfonyl)pyrimidin-2-amine (Intermediate 69, 1.0 g, 2.63 mmol) in DMF (2 mL) was added slowly to the reaction mixture, and the mixture was stirred for 1 h. Water was added to the reaction mixture (˜6 mL) and the solid that formed was filtered and dried to yield 5-bromo-N-(3-chloro-4-fluorophenyl)-4-(3,5-dimethyl-1H-pyrazol-1-yl)pyrimidin-2-amine (800 mg),

MS(ES): 396 (M) and 398 (M+2) for C₁₅H₁₂BrClFN₅

400 MHz, DMSO-d₆: δ 2.19 (s, 3H), 2.34 (s, 3H), 6.14 (s, 1H), 7.38 (t, J=9.08 Hz, 1H), 7.54-7.58 (m, 1H), 7.96 (dd, J=2.48, 6.68 Hz, 1H), 8.83 (s, 1H), 10.24 (s, 1H).

The following intermediates were prepared using the general method described above for Intermediate 112 using 5- bromo-2-[N-(3-chloro-4-fluorophenyl)]-4-(methylsulfonyl)pyrimidin-2-amine (Intermediate 69), sodium hydride and the starting material (SM) indicated.

| Int | Compound | Data | SM |
|---|---|---|---|
| 113 | 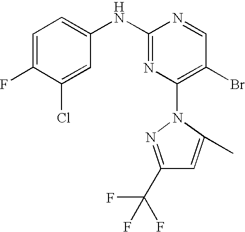 5-bromo-N-(3-chloro-4- fluorophenyl)-4-[5-methyl-3- (trifluoromethyl)-1H-pyrazol-1- yl]pyrimidin-2-amine | MS(ES): 450 (M) for C₁₅H₉BrClF₄N₅. 300 MHz, CDCl₃: δ 2.45 (s, 3H), 6.51 (s, 1H), 7.14 (t, J = 8.70 Hz, 1H), 7.27- 7.30 (m, 1H), 7.75 (dd, J = 2.61, 6.39 Hz, 1H), 8.70 (s, 1H). | (5-Methyl-3- trifluoro- methyl-1H- pyrazole) |
| 114 | 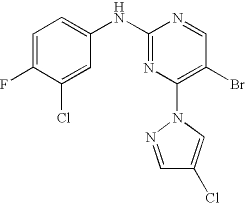 5-bromo-N-(3-chloro-4- fluorophenyl)-4-(4-chloro-1H- pyrazol-1-yl)pyrimidin-2- amine | MS(ES): 404 (M + 2) for C₁₃H₇BrCl₂FN₅. 400 MHz, CDCl₃: δ 7.16 (d, J = 8.56 Hz 1H), 7.32-7.36 (m, 1H), 7.78 (m, 2H), 8.38 (s, 1H), 8.65 (s, 1H). | 4-Chloro-1H- pyrazole |
| 115 | 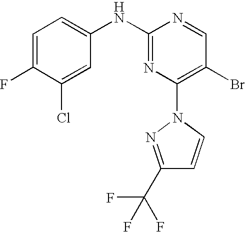 5-bromo-N-(3-chloro-4- fluorophenyl)-4-[3-(trifluoro- methyl)-1H-pyrazol-1-yl]pyrimidin-2- amine | MS (ES): 436 (M) and 438 (M + 2) for C₁₄H₇BrClF₄N₅. 300 MHz, DMSO-d₆: δ 7.14 (s, 1H), 7.28 (t, J = 9.06 Hz 1H), 7.61 (d, J = 8.64 Hz 1H), 8.00 (br s, 1H), 8.62 (br s, 1H), 8.91 (br s, 1H), 10.83 (br s, 1H). | 3-Trifluoro- methyl-1H- pyrazole |
| 116 | 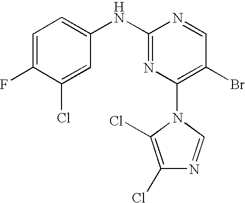 5-bromo-N-(3-chloro-4- fluorophenyl)-4-(4,5-dichloro- 1H-imidazol-1-yl)pyrimidin-2- amine | MS(ES): 436 (M) and 438 (M + 2) for C₁₃H₆BrCl₃FN₅. 300 MHz, DMSO-d₆: δ 7.39 (t, J = 9.06 Hz, 1H), 7.57-7.60 (m, 1H), 7.92 (d, J = 5.85 Hz 1H), 8.28 (s, 1H), 9.0 (s, 1H), 10.55 (br s, 1H). | 4,5-Dichloro- 1H-imidazole |
| 117 | 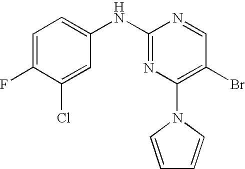 5-bromo-N-(3-chloro-4- fluorophenyl)-4-(1H-pyrrol-1- yl)pyrimidin-2-amine | MS(ES): 367 (M) for C₁₄H₉BrClFN₄. 300 MHz DMSO-d₆: δ 6.36 (s, 2H), 7.37 (t, J = 9.12 Hz, 1H), 7.60 (br s, 3H), 8.01 (dd, J = 6.69, 2.37 Hz, 1H), 8.75 (s, 1H), 10.18 (s, 1H). | 1H-Pyrrole |
| 118 | 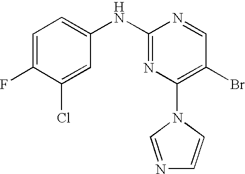 5-bromo-N-(3-chloro-4- fluorophenyl)-4-(1H-imidazol- 1-yl)pyrimidin-2-amine | MS(ES): 368 (M) for C₁₃H₈BrClFN₅. 1H NMR (300 MHz, DMSO-D6) δ ppm 7.18 (s, 1H) 7.40 (t, J = 9.14 Hz, 1H) 7.55-7.69 (m, 1H) 7.82 (s, 1H) 7.98 (dd, J = 6.78, 2.64 Hz, 1H) 8.39 (s, 1H) 8.86 (s, 1H) 10.33 (s, 1H). | 1H-Imidazole |

The following intermediates were prepared using the general method described above for Intermediate 112 using sodium hydride and the starting materials (SM) indicated.

| Int | Compound | Data | SM |
|---|---|---|---|
| 119 | 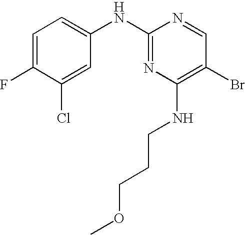 5-bromo-N²-(3-chloro-4- fluorophenyl)-N⁴-(3- methoxypropyl)pyrimidine- 2,4-diamine | MS: ES+ 390 for C₁₄H₁₅BrClFN₄O 1H NMR (300 MHz, DMSO-d₆) δ ppm 1.75- 1.90 (m, 2H) 3.24 (s, 3H) 3.37-3.53 (m, 4H) 7.11 (t, J = 5.56 Hz, 1H) 7.28 (t, J = 9.14 Hz, 1H) 7.57 (ddd, J = 9.09, 4.29, 2.64 Hz, 1H) 8.04 (s, 1H) 8.11 (dd, J = 6.97, 2.64 Hz, 1H) 9.45 (s, 1H) | 3-methoxy propylamine and Intermediate 69 |
| 120 | 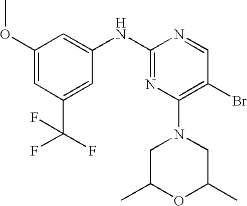 5-bromo-4-(2,6- dimethylmorpholino)-N- (3-methoxy-5-(trifluoro methyl)phenyl) pyrimidin-2-amine | MS: ES+ 462 for C₁₈H₂₀BrF₃N₄O₂ 1H NMR (300 MHz, DMSO-d₆) δ ppm 1.12 (d, J = 6.22 Hz, 6H) 2.54-2.70 (m, 2H), 3.67 (dd, J = 8.29, 6.41 Hz, 2H) 3.79 (s, 3H) 4.13 (d, J = 12.62 Hz, 2H) 6.79 (s, 1H) 7.51 (s, 1H) 7.84 (s, 1H) 8.24-8.28 (m, 1H) 9.82 (s, 1H) | 2,6-dimethyl morpholine and Intermediate 70 |
| 121 | 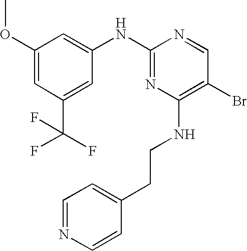 5-bromo-N²-(3-methoxy-5- (trifluoromethyl)phenyl)- N⁴-(2-(pyridine-4-yl) ethyl)pyrimidine-2,4- diamine | MS: ES+ 469 for C₁₉H₁₇BrF₃N₅O 1H NMR (300 MHz, DMSO-d₆) δ ppm 2.91 (t, J = 7.35 Hz, 2H) 3.60-3.72 (m, 2H) 3.75 (s, 3H) 6.76 (s, 1H) 7.13-7.27 (m, 3H) 7.63 (s, 1H) 7.76 (s, 1H) 8.07 (s, 1H) 8.39-8.47 (m, 2H) 9.56 (s, 1H) | 2-(pyridine- 4-yl)ethan amine and Intermediate 70 |
| 122 | 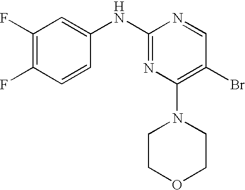 5-bromo-N-(3,4- difluorophenyl)-4- morpholinopyrimidin-2- amine | MS: ES+ 372 for C₁₄H₁₃BrF₂N₄O 1H NMR (300 MHz, DMSO-d₆) δ ppm 3.53- 3.61 (m, 4H) 3.67-3.76 (m, 4H) 7.21-7.45 (m, 2H) 7.86 (ddd, J = 14.03, 7.44, 2.45 Hz, 1H) 8.26 (s, 1H) 9.71 (s, 1H) | Morpholine and Intermediate 71 |

### Intermediate 123: N-(3-chloro-4-fluorophenyl)-4-methylsulfonyl-5-pyrimidin-5-ylpyrimidin-2-amine

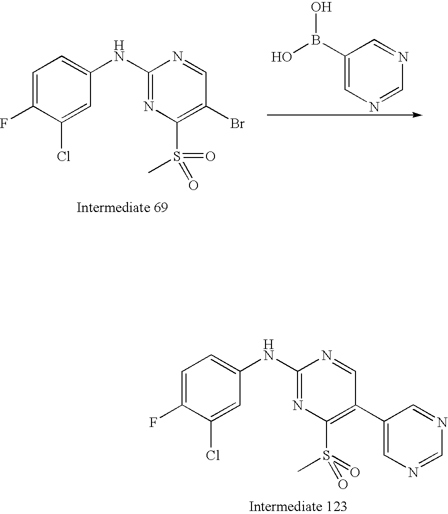

A suspension of Intermediate 69 (1.05 mmol, 400 mg), pyrimidine-5-boronic acid (1.68 mmol, 208 mg), tris(dibenzyledeneacetone)dipalladium(0) (10 mol %, 0.1 mmol, 96 mg), 2-dicyclohexyl phosphino-2′,4′,6′-triiso-propyl-1,1′-biphenyl (30 mol %, 0.3 mmol, 148 mg) and sodium carbonate (1.05 mmol, 112 mg) in acetonitrile/water (4:1) was heated to 90° C. for 30 min in an oil bath. The reaction mixture was diluted with ethyl acetate (10 mL) and the organic layer was separated, dried over sodium sulfate, filtered and concentrated. The crude compound was purified by silica gel column chromatography using chloroform:methanol (9:1) as an eluent in 20% yield.

MS(ES): 380 (M+1) for C₁₅H₁₁ClFN₅O₂S.

300 MHz DMSO-d₆: δ 3.4 (s, 3H), 7.44 (t, J=9.2 Hz, 1H), 7.63 (brs, 1H), 8.0 (br s, 1H), 8.86 (s, 1H), 8.89 (s, 2H), 9.21(s, 1H), 10.63 (s, 1H).

### Intermediate 124: Ethyl 5-{2-[(3-chloro-4-fluorophenyl)amino]-4-(methylsulfonyl)pyrimidin-5-yl}pyridine-3-carboxylate

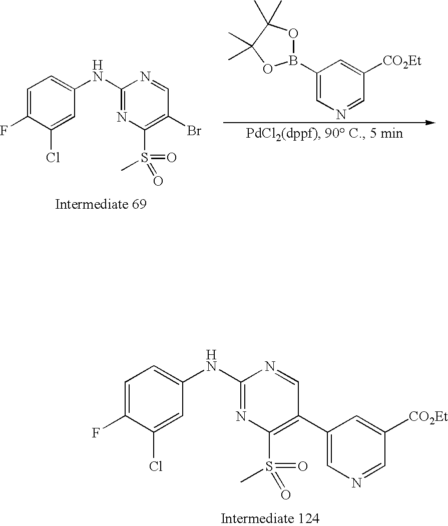

A suspension of Intermediate 69 (1.31 mmol, 0.5 g), ethyl 5-(4,4,5,5-tetramethyl-1,3,2-dioxaborolan-2-yl)pyridine-3-carboxylate (1.38 mmol, 382 mg), [1,1′-bis(diphenylphosphino)ferrocene]dichloropalladium (II) complex with CH₂Cl₂ (10 mol %, 0.13 mmol, 107 mg), 2-dicyclohexyl phosphino-2′,4′,6′-triisopropyl-1,1′-biphenyl (30 mol %, 0.394 mmol, 188 mg) and sodium carbonate (2.62 mmol, 279 mg) in acetonitrile/water (20 mL: 5 mL) was heated to 90° C. for 5 min in an oil bath under inert atmosphere. After completion of the reaction, as monitored by TLC, the reaction mixture was diluted with EtOAc (30 mL). The organic layer was separated, dried over sodium sulfate, filtered and concentrated. The residue was purified by silica gel column chromatography using hexane:ethyl acetate (3:2) as an eluent. The title compound was obtained in 51% combined yield (0.30 g, 1.33 mmol).

MS (ES): 451 (M+1) for C₁₉H₁₆ClFN₄O₄S

¹H NMR (300 MHz, DMSO-d₆): δ 1.34 (t, J =7.08 Hz, 3H), 3.38 (s, 3H), 4.37 (q, J=7.0 Hz, 2H), 7.44 (t, J=8.97 Hz, 1H), 7.64 (m, 1H), 8.0-8.01 (m, 1H), 8.39 (s, 1H), 8.83 (s, 1H), 8.87 (d, J=2.01 Hz, 1H), 9.11 (d, J=1.83 Hz, 1H), 10.59 (s, 1H).

### Intermediate 125: Ethyl (2E)-3-(3-{2-[(3-chloro-4-fluorophenyl)amino]-4-(methylsulfonyl)pyrimidin-5-yl}phenyl)prop-2-enoate

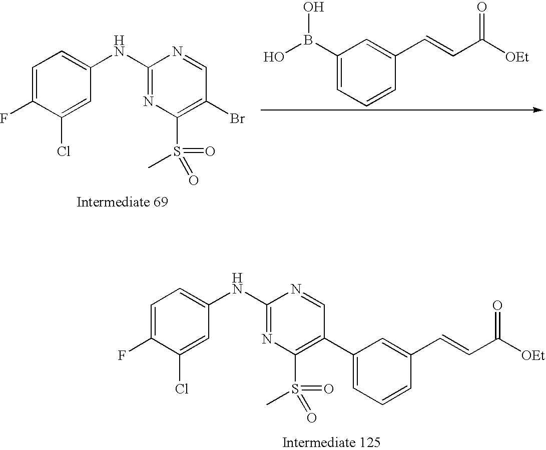

A suspension of Intermediate 69 (2.11 mmol, 800 mg), {3-[(1E)-3-ethoxy-3-oxoprop-1-en-1-yl]phenyl}boronic acid (2.32 mmol, 510 mg), tris(dibenzylideneacetone)dipalladium(0) (10 mol %, 0.21 mmol, 192 mg), 2-dicyclohexylphosphino-2′,4′,6′-triisopropyl-1,1′-biphenyl 30 mol %, 0.63 mmol, 300 mg) and sodium carbonate (3.15 mmol, 330 mg) in acetonitrile/water (40 mL; 4:1 mixture) was heated to 90° C. for 15-20 min in an oil bath. Acetonitrile was evaporated from the reaction mixture and water was added. The mixture was extracted with ethyl acetate (10 mL), and the organic layer was separated, dried over sodium sulfate, filtered and concentrated. The crude compound was purified by silica gel column chromatography using hexane-ethyl acetate (13:7) as an eluent to yield the title compound as a pale yellow solid in 52% yield (0.52 g, 1.08 mmol).

MS(ES): 476 (M+1) for C₂₂H₁₉ClFN₃O₄S

¹H-NMR (400 MHz, DMSO-d₆): δ 1.26 (t, J=7.04 Hz, 3H), 3.36 (s, 3H), 4.20 (q, J=7.08 Hz, 2H), 6.68 (d, J=16.04 Hz, 1H), 7.43 (t, J=9.08 Hz, 1H), 7.50 (t, J=7.64 Hz, 1H), 7.56 (d, J=7.76 Hz, 1H), 7.64 (ddd, J=2.72, 4.12, 9.05 Hz, 1H), 7.69 (d, J=16.04 Hz, 1H), 7.77 (d, J=7.68 Hz, 1H), 7.84 (s, 1H), 8.02 (dd, J=2.48, 6.72 Hz, 1H), 8.80 (s, 1H), 10.51 (s, 1H).

### Intermediate 126: Tert-butyl 2-[[[5-bromo-2-[(3-chloro-4-fluorophenyl)amino]pyrimidin-4-yl]amino]methyl]benzimidazole-1-carboxylate

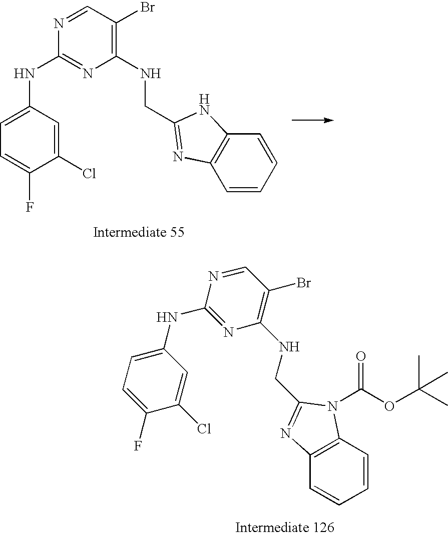

To a stirred solution of Intermediate 55 (1 g, 2.23 mmol) and triethylamine (1.24 mL, 8.93 mmol) in dichloromethane (20 mL) under nitrogen atmosphere was added di-t-butyldicarbonate(1.46 g, 6.7 mmol) at 5-10° C. The reaction mixture was stirred overnight at room temperature, then diluted with water (20 mL) and extracted with dichloromethane (2×20 mL). The combined extracts were washed with water (2×20 mL), brine solution, dried over sodium sulfate, filtered and concentrated to give the title compound as a white solid in 83% yield (1.2 g, 1.85 mmol).

MS(ES): 547 (M) and 549 (M+2) for C₂₃H₂₁BrClFN₆O₂.

¹H NMR (300 MHz) DMSO-d₆: δ 1.67 (s, 9H), 5.01 (d, J=5.46 Hz, 2H), 7.05 (t, J=9.12 Hz, 1H), 7.27-7.36 (m, 3H), 7.57 (t, J=4.5 Hz, 1H), 7.66 (d, J=7.44 Hz, 1H), 7.78 (t, J=2.4 Hz, 1H), 7.92 (d, J=7.59 Hz, 1H), 8.14 (s, 1H), 9.44 (s, 1H).

### Intermediate 127: 2-(3-Chloro-4-fluorophenylamino)-4-(3-methoxypropylamino)pyrimidine-5-carbonitrile

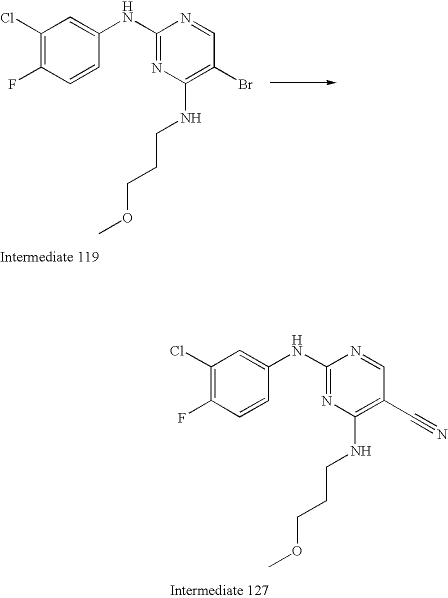

A stirred suspension of Intermediate 119 (4.01 g, 10.3 mmol), zinc powder (168 mg, 2.57 mmol), zinc cyanide (784 mg, 6.67 mmol), tris(dibenzylideneacetone)dipalladium (0) (188 mg, 0.210 mmol), 1,1′-bis(diphenylphosphino)ferrocene (230 mg, 0.410 mmol), and zinc acetate (75 mg, 0.41 mmol) in degassed N,N-dimethylformamide (25 mL) was prepared. The vessel was purged with nitrogen gas for one minute. The mixture was placed under an atmosphere of nitrogen atmosphere and heated to 100 degrees C. A check of progress by LCMS after about an hour indicated complete conversion to desired product. Stirring continued while the mixture was allowed to cool to room temperature; the mixture was then diluted with small volumes of water. The dark solution became cloudy with addition of water. Larger volumes were added until a maximum of precipitation was reached. Stirring continued for ten minutes. The solid was collected and washed with water. Normal-phase chromatography (0-3% methanol in methylene chloride) was used to isolate the pure product (3.3 g, 95%).

MS: ES+ 336 for C₁₅H₁₅ClFN₅O.

1H NMR (300 MHz, DMSO-d₆) δ ppm 1.74-1.90 (m, J=6.76, 6.76, 6.64, 6.40 Hz, 2H) 3.22 (s, 3H) 3.39 (t, J=6.22 Hz, 2H) 3.46 (q, J=6.53 Hz, 2H) 7.33 (t, J=9.04 Hz, 1H) 7.60 (ddd, J=9.04, 4.24, 2.73 Hz, 1H) 7.86 (br. s., 1H) 8.11 (dd, J=6.78, 2.45 Hz, 1H) 8.36 (s, 1H) 9.99 (br. s., 1H)

### Intermediate 128: 2-(3-Chloro-4-fluorophenylamino)-4-(3-methoxypropylamino)pyrimidine-5-carbothioamide

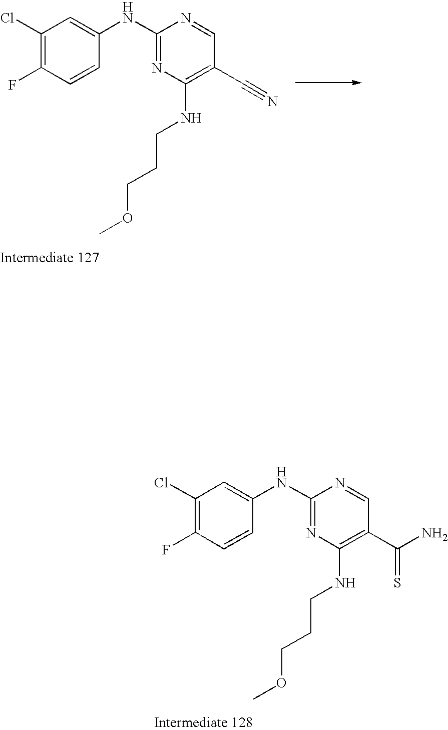

A stirred solution of Intermediate 127 (750 mg, 2.2 mmol) in methanol (4 mL) and N,N-dimethylformamide (4 mL) was prepared under ambient conditions. The reaction vessel was purged with nitrogen, and the contents were placed under an atmosphere of nitrogen. The mixture was heated to 90 degrees C. At that time 1 mL of a 22 wt % aqueous solution of ammonium sulfide (about 3 mmol ammonium sulfide) was added slowly by syringe. Volumes of a similar size were added every thirty minutes until conversion to desired product was complete as monitored by LCMS. The mixture was allowed to cool to room temperature with stirring. Water (5 mL) was then added very slowly with stirring, precipitating a grey solid. This was collected, washed with water and dried to yield the title compound (768 mg, 93%).

MS: ES+ 369 for C₁₅H₁₇ClFN₅OS.

1H NMR (300 MHz, DMSO-d₆) δ ppm 1.84 (qd, J=6.22, 6.03 Hz, 2H) 3.24 (s, 3H) 3.44 (t, J=6.03 Hz, 2H) 3.52 (q, J=5.90 Hz, 2H) 7.32 (t, J=9.04 Hz, 1H) 7.52-7.70 (m, 1H) 8.22 (d, J=4.90 Hz, 1H) 8.36 (s, 1H) 9.27 (br. s., 1H) 9.39 (br. s., 1H) 9.64 (br. s., 1H) 9.80 (br. s., 1H).

### Intermediate 129: 5-bromo-N-(3-chloro-4-fluorophenyl)-4-(piperazin-1-yl)pyrimidin-2-amine hydrochloride

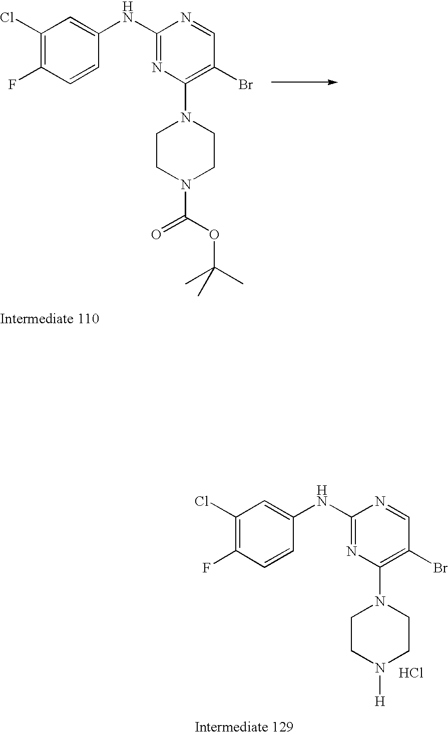

To a stirred solution of Intermediate 110 (500 mg, 1.30 mmol) in 1,4- dioxane (10 mL) under nitrogen atmosphere was added 4N hydrochloric acid in 1,4-dioxane (3 mL) dropwise. The reaction mixture was stirred at room temperature for 24 h. The reaction mixture was concentrated to give the hydrochloride salt title compound as a white solid (320 mg).

MS(ES): 386 (M) 388 (M+2) for C₁₄H₁₄BrClFN₅

1H NMR (300 MHz, DMSO- d6) δ ppm 3.22 (s, 4H) 3.67-3.91 (m, 4H) 7.33 (t, J=9.04 Hz, 1H) 7.41-7.63 (m, 1H) 8.00 (d, J=6.03 Hz, 1H) 8.33 (s, 1H) 9.34 (d, J=20.91 Hz, 1H) 9.83 (s, 1H).

### Intermediate 130: 5-Bromo-N-(3-chloro-4-fluorophenyl)-4-(4-(methylsulfonyl)piperazin-1-yl)pyrimidin-2-amine

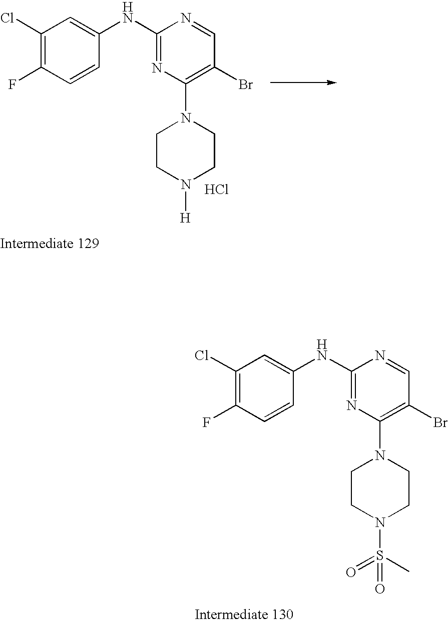

A solution of methanesulfonyl chloride (30 mg, 0.26 mmol) was added to Intermediate 129 (100 mg, 0.26 mmol), triethylamine (0.054 ml, 0.39 mmol) and methylene chloride (1.5 ml) under nitrogen. The resultant mixture was stirred for 1 h and concentrated under vacuum. The residue was chromatographed using 40-70% ethyl acetate/hexane to yield the title compound (88 mg).

MS(ES): 386 (M) 388 (M+2) for C₁₅H₁₆BrClFN₅O₂S

1H NMR (300 MHz, DMSO- d6) δ ppm 2.92 (s, 3H) 3.12-3.30 (m, 4H) 3.55-3.84 (m, 4H) 7.33 (t, J=9.14 Hz, 1H) 7.47-7.71 (m, 1H) 7.93-8.11 (m, 1H) 8.30 (s, 1H) 9.74 (s, 1H).

### Intermediate 131: Methyl 6-bromoquinoline-3-carboxylate

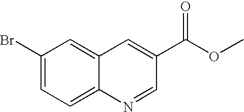

6-Bromoquinoline-3-carboxylic acid (2.5 g, 9.92 mmol) was suspended in methyl alcohol (40.2 ml, 991.81 mmol) and treated with sulfuric acid (2.64 ml, 49.59 mmol). The reaction was refluxed overnight. TLC analysis of a small aliquot showed complete reaction. The reaction was cooled to room temperature and concentrated at reduced pressure. The residue was diluted with EtOAc and carefully neutralized with a solution of sodium carbonate. The biphasic suspension was diluted with EtOAc/DCM until all solid dissolved. The layers were then separated, and the organic was washed with brine and dried over sodium sulfate. The solvent was removed at reduced to afford the desired product with good purity. It was used without further purification.

MS(ES): 266 (M), 268 (M+2) for C₁₁H₈BrNO₂

1H NMR (300 MHz, DMSO-D6) δ ppm 3.95 (s, 3H) 8.03 (d, J=1.13 Hz, 2H) 8.52 (s, 1H) 8.99 (d, J=2.07 Hz, 1H) 9.31 (d, J=2.07 Hz, 1H).

### Intermediate 132: (Z)-ethyl 3-(dimethylamino)-2-(2-fluoro-5-iodobenzoyl)acrylate

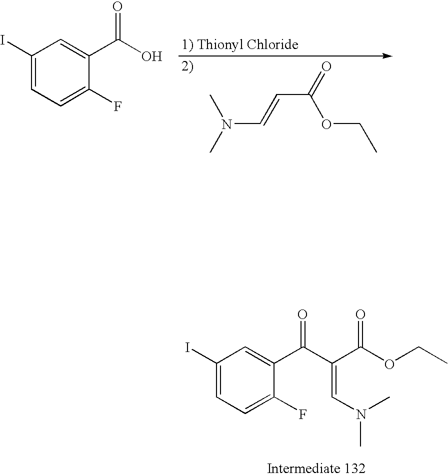

2-Fluoro-5-iodobenzoic acid (3.76 mmol, 1 g) was treated with thionyl chloride (3 mL) and DMF (2 drops). The reaction mixture was heated at 70° C. for 1.5 h to form the acid chloride, then cooled and concentrated under reduced pressure. Toluene was added, and the mixture was concentrated again. The crude acid chloride was dissolved in toluene and treated with triethylamine (3.76 mmol, 0.524 mL) and ethyl 3-(dimethylamino)acrylate (4.89 mmol, 0.700 g). The reaction mixture was heated to 90° C. for 1.5 h. The reaction mixture was filtered and the resulting solution was purified using flash column chromatography (silica, 2.5:1 hexanes/ethyl acetate) to give the desired product in 47% yield. (1.79 mmol, 0.7 g)

MS(ES): 391.9 (M+1) for C₁₄H₁₅FINO₃.

¹H NMR (300 MHz) DMSO-d₆ δ: 0.95 (t, J=7.14 Hz, 3H), 2.89 (br s, 3H), 3.33 (br s, 3H), 4.0(q, J=7.08 Hz, 2H), 6.80 (t, J=1.17 Hz, 1H), 7.64-7.69(m, 1H), 7.79 (m, 1H), 7.87 (br s, 1H).

### Intermediate 133: Ethyl 6-iodo-1-(2-methoxyethyl)-4-oxo-1,4-dihydroquinoline-3-carboxylate

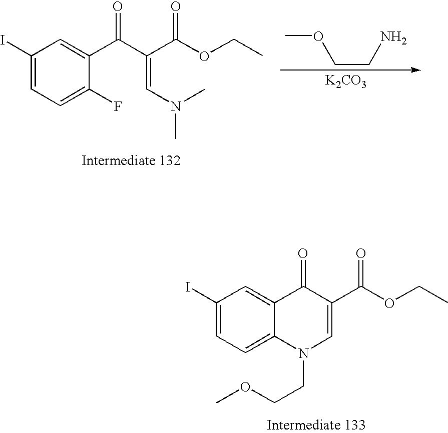

A suspension of (Z)-ethyl 3-(dimethylamino)-2-(2-fluoro-5-iodobenzoyl)acrylate Intermediate 132 (1.79 mmol, 0.7 g) in ethanol (3 mL) at room temperature was treated with 2-methoxyethylamine (2.26 mmol, 0.17 mL). The reaction mixture was stirred until a yellow solution resulted and concentrated under reduced pressure after 1 h. Potassium carbonate (2.69 mmol, 0.372 g) and DMF (2 mL) were added. The reaction mixture was heated to 70° C. for 3 h, then cooled to room temperature and allowed to stand overnight. The reaction mixture was poured into water. The solid that formed was collected by filtration, washed with water and dried under vacuum for 4 h to give desired product in 80% yield (1.44 mmol, 0.580 g).

MS(ES): 402 (M+1) for C₁₅H₁₆INO₄.

¹H NMR (400 MHz) DMSO-d₆ δ: 1.27 (t, J=7.08 Hz, 3H), 3.32 (s, 3H), 3.64 (t, J=4.84 Hz, 2H), 4.22 (q, J=7.08 Hz, 2H), 4.54 (t, J=4.92 Hz, 2H), 7.68 (d, J=9.00 Hz, 1H), 8.05 (dd, J=8.94, 2.16 Hz, 1H), 8.49 (d, J=2.16 Hz, 1H), 8.59 (s, 1H).

### Intermediate 134: Ethyl 1-(2-methoxyethyl)-4-oxo-6-(4,4,5,5-tetramethyl-1,3,2-dioxaborolan-2-yl)-1,4-dihydroquinoline-3-carboxylate

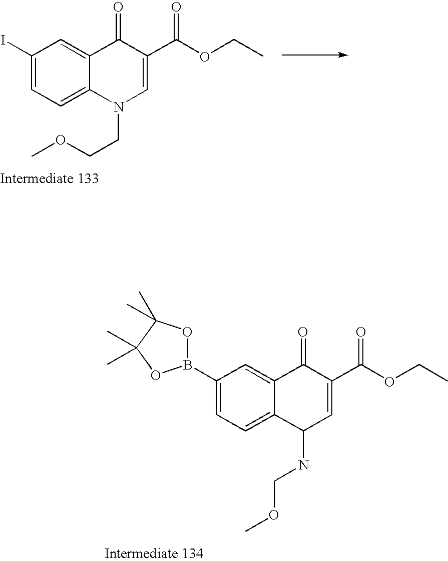

Ethyl 6-iodo-1-(2-methoxyethyl)-4-oxo-1,4-dihydroquinoline-3-carboxylate (Intermediate 133, 400 mg, 1.00 mmol) was suspended in dioxane (4 mL) and degassed with nitrogen for 10 minutes at which time bis(pinacolato)diboron (506 mg, 1.99 mmol), 1,1′-bis(diphenylphosphino)ferrocene-palladium dichloride (41.0 mg, 0.05 mmol), and potassium acetate (294 mg, 2.99 mmol) were added. After the reaction was heated at 90° C. overnight, LC/MS showed completion of the reaction. The reaction was then cooled to room temperature, diluted with DCM and filtered through a pad of diatomaceous earth. The filtrate was concentrated at reduced pressure and quickly purified by silica gel chromatography with 8% MeOH in DCM to yield the title compound.

MS(ES): 402 (M+1) for C₂₁H₂₈BNO₆

1H NMR (300 MHz, DMSO-D6) δ ppm 1.20-1.45 (m, 15H) 3.17-3.27 (s, 3H) 3.67 (t, J=4.71 Hz, 2H) 4.24 (q, J=6.97 Hz, 2H) 4.57 (t, J=4.71 Hz, 2H) 7.75-7.91 (m, 1H) 7.90-8.04 (m, 1H) 8.49-8.74 (m, 2H).

The following intermediate was prepared using the general method described above for Intermediate 134 using bis(pinacolato)diboron, 1,1′-bis(diphenylphosphino)ferrocene-palladium dichloride, potassium acetate and the starting material (SM) indicated.

| Int | Compound | Data | SM |
|---|---|---|---|
| 135 | 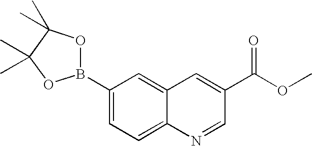 Methyl 6-(4,4,5,5-tetramethyl-1,3,2- dioxaborolan-2-yl)quinoline-3-carboxylate | ¹H NMR (300 MHz, DMSO-D6) δ ppm 1.34 (s, 12H) 3.95 (s, 3H) 8.08 (s, 2H) 8.58 (s, 1H) 9.10 (d, J = 1.88 Hz, 1H) 9.35 (d, J = 2.07 Hz, 1H) | Intermediate 131 |

### Intermediate 136: 4-(5-Bromo-2-chloro-pyrimidin-4-yl)-morpholine

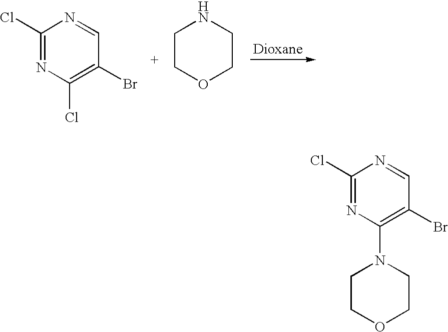

To a stirred solution of 2,4-dichloro-5-bromopyrimidine (22 mmol, 5 g) in dioxane (100 mL), at room temperature under nitrogen atmosphere, was added morpholine (29.2 mmol, 2.54 g). The reaction mixture was stirred overnight at room temperature, diluted with ethyl acetate (50 mL) and the resulting white solid formed was removed by filtration. The filtrate was concentrated and the resulting residue was purified by column chromatography (using 60-120 mesh silica gel and 4% of ethyl acetate in petroleum ether) to yield the title compound. (4.7 g).

MS(ES): 278 (M) and 280 (M+2) for C₈H₉BrClN₃O.

¹H-NMR (300 MHz, CDCl₃): δ 3.79 (s, 8H), 8.27 (s, 1H).

### Intermediate 137: 5-(2-Chloro-4-morpholin-4-yl-pyrimidin-5-yl)-nicotinic acid ethyl ester

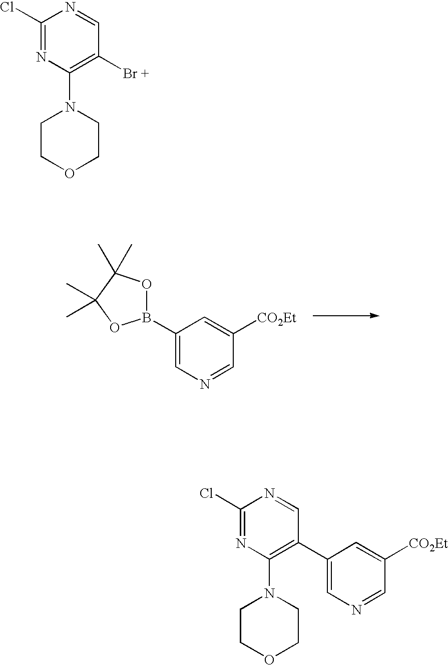

A suspension of Intermediate 136 (3.5 mmol, 1 g), 5-(4,4,5,5-Tetramethyl-[1,3,2]dioxaborolan-2-yl)-nicotinic acid ethyl ester (3.6 mmol, 1.01 g), [1,1′-bis(diphenylphosphino)ferrocene]dichloropalladium(II) complex with CH₂Cl₂ (10 mol %, 0.35 mmol, 285 mg) and sodium carbonate (3.5 mmol, 370 mg) in acetonitrile/water (20 mL:5 mL) was degassed and heated to 90° C. for 20 min under an inert atmosphere. The solvent was removed in vacuo and the crude mixture was taken in CHCl₃ (30 mL). It was then washed with water and brine. The organic layer was separated, dried over sodium sulfate, filtered and concentrated. The crude material was purified by silica gel column chromatography, using Hexane:Ethyl Acetate (3:1) as eluent to get Intermediate 137 (0.69 g).

MS (ES) : 349 (M+1) for C₁₆H₁₇ClN₄O₃.

¹H-NMR (300 MHz, DMSO-d₆): δ 0.81 (t, J=6.54 Hz, 3H), 3.24-3.25 (m, 4H), 3.49-3.50 (m, 4H), 4.37 (q, J=7.05 Hz, 2H), 8.20 (s, 1H), 8.37 (s, 1H), 8.91 (d, J=1.98 Hz, 1H), 9.07 (d, J=1.80 Hz, 1H).

### Intermediate 138: (5-Bromo-4-methylsulfanyl-pyrimidin-2-yl)-(3,5-difluoro-phenyl)-amine

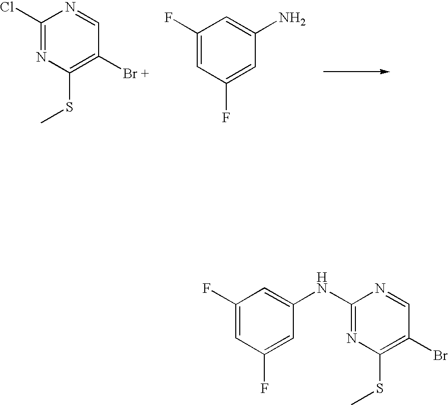

5-Bromo-2-chloro-4-(methylthio)pyrimidine (8.3 mmol, 2 g) was suspended in n-BuOH (20 mL) and treated with 3,5-difluoroaniline (9.1 mmol, 1.18 g), HCl in dioxane (4 mL) was added under nitrogen atmosphere and the mixture was refluxed at 100° C. for 3 h. The reaction was cooled to room temperature, diluted with diethyl ether and the solid was filtered and dried to afford Intermediate 138.

MS(ES): 333.8 (M+2) for C₁₁H₈BrF₂N₃S.

¹H-NMR (400 MHz, DMSO-d₆): δ 2.58 (s, 3H), 6.78 (tt, J=2.32, 9.27 Hz, 1H), 7.50 (dd, J=2.20, 10.36 Hz, 2H), 8.40 (s, 1H), 10.19 (s, 1H).

### Intermediate 139: (5-Bromo-4-methanesulfonyl-pyrimidin-2-yl)-(3,5-difluoro-phenyl)-amine

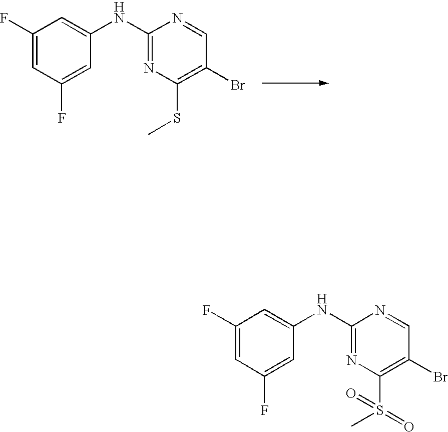

Intermediate 138 (3 mmol, 1 g) was suspended in acetone (10 mL), cooled to 0° C. and 3-chloroperoxybenzoic acid (15 mmol, 2.59 g) was added portion wise. The suspension became a clear solution after stirring at 0° C. for 30 min The reaction mixture was then allowed to warm up slowly to room temperature and stirred for 5 h. The reaction mixture pH was raised to 8 with the addition of aq. NaHCO₃ solution (50 mL), extracted with ethyl acetate (3×10 mL), combined extracts were washed with brine, dried over Na₂SO₄, filtered and concentrated to get crude Intermediate 139 (1 g).

MS(ES): 363.9 (M) and 365 (M+1) for C₁₁H₈BrF₂N₃O₂S.

¹H-NMR (300 MHz, DMSO-d₆): δ 3.54 (s, 3H), 6.86 (t, J=9.24 Hz, 1H), 7.44 (d, J=8.82 Hz, 2H), 8.99 (s, 1H), 10.72 (s, 1H).

### Intermediate 140: (5-Bromo-4-morpholin-4-yl-pyrimidin-2-yl)-(3,5-difluoro-phenyl)-amine

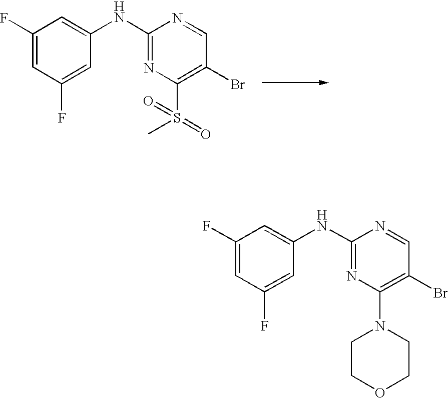

Intermediate 139 (2.7 mmol, 1 g) was suspended in NMP (10 mL), treated with N,N-diisopropylethylamine (3.4 mmol, 0.56 mL), morpholine (30 mmol, 263 mg) in a sealed tube. The reaction was heated at 90° C. for 30 min then cooled to room temperature, added to water and stirred for 15 min. The precipitate was filtered and dried to afford Intermediate 140.

MS(ES): 371 (M) and 373 (M+2) for C₁₄H₁₃BrF₂N₄O.

¹H-NMR (300 MHz, DMSO-d₆): δ 3.59 (d, J=3.93 Hz, 4H), 3.72 (s, 4H), 6.72 (t, J=9.36 Hz, 1H), 7.45 (d, J=10.32 Hz, 2H), 8.30 (s, 1H), 9.91 (s, 1H).

### Intermediate 141: (5-Bromo-4-methoxy-pyrimidin-2-yl)-(3,5-dimethyl-phenyl)-amine

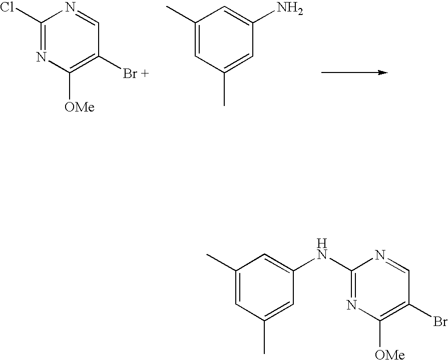

To a stirred solution of 5-bromo-2-chloro-4-methoxypyrimidine (17.9 mmol, 4 g) and 3,5-dimethylaniline (18.8 mmol, 2.2 g) in acetonitrile (50 mL), 4 M HCl in 1,4-dioxane (5 mL) was added drop wise. The resulting solution was refluxed at 100° C. with constant stirring. The solvent was removed in vacuo and basified with 10% NaHCO₃ solution, extracted with EtOAc (2×150 mL). The combined organic layer was washed with water and brine, dried over sodium sulfate and concentrated under vacuo to yield Intermediate 141 as a white solid (9.7 mmol, 3 g, 54%).

MS(ES): 310 (M+2) for C₁₃H₁₄BrN₃O.

¹H-NMR (400 MHz, DMSO-d₆): δ 2.23 (s, 6H), 3.99 (d, J=1.84 Hz, 3H), 6.62 (s, 1H), 7.34 (s, 2H), 8.35 (d, J=2.0 Hz, 1H), 9.63 (s, 1H).

### Intermediate 142: 5-Bromo-2-(3,5-dimethyl-phenylamino)-pyrimidin-4-ol

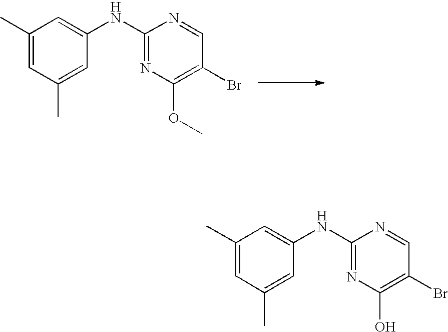

A mixture of Intermediate 141 (9.7 mmol, 3 g) and aq. sodium thiomethoxide (38.9 mmol, 18 mL, 21% w/v,) and DMF (75 mL) was heated at 60° C. for 2 h; cooled to room temperature, poured into water (150 mL) and acidified with 1.5 N HCl solution. The precipitated solid was filtered to yield Intermediate 142 as off-white solid (2.5 g).

MS(ES): 294 (M) and 296 (M+2) for C₁₂H₁₂BrN₃O.

¹H-NMR (400 MHz, DMSO-d₆): δ 2.23 (s, 6H), 6.69 (s, 1H), 7.14 (s, 2H), 8.04 (s, 1H), 8.79 (br s, 1H), 11.33 (br s, 1H).

### Intermediate 143: (5-Bromo-4-chloro-pyrimidin-2-yl)-(3,5-dimethyl-phenyl)-amine

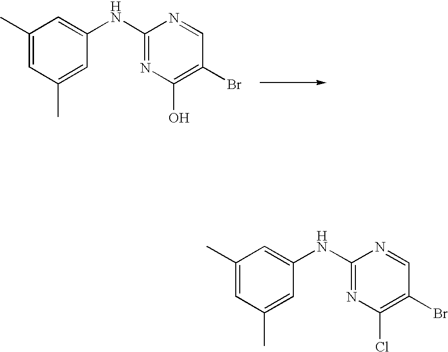

A solution of Intermediate 142 (8.4 mmol, 2.5 g) in phosphorus oxychloride (13 mL) was heated to reflux for 45 min, cooled to RT, poured carefully onto a mixture of ice (100 mL) and saturated aq. NaHCO₃ solution (20 mL) with constant stirring. It was further extracted with ethyl acetate (2×150 mL). The combined organic layer was washed with water and brine, dried over sodium sulfate and concentrated under vacuo to yield Intermediate 143 (1.7 g).

MS(ES): 312 (M) and 314 (M+2) for C₁₂H₁₁BrClN₃.

¹H-NMR (400 MHz, DMSO-d₆): δ 2.23 (s, 6H), 6.67 (s, 1H), 7.27 (s, 2H), 8.66 (s, 1H), 10.05 (s, 1H).

### Intermediate 144: (5-Bromo-4-morpholin-4-yl-pyrimidin-2-yl)-(3,5-dimethyl-phenyl)-amine

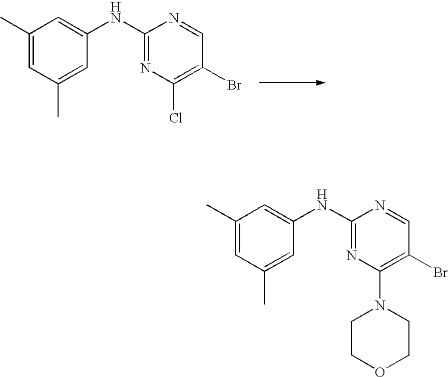

A mixture of Intermediate 143 (5.4 mmol, 1.7 g) and morpholine (10.8 mmol, 0.94 g) in dioxane (40 mL) was stirred at room temperature for 18 h. Then the reaction mixture was concentrated under vacuo to yield white solid. The solid was dissolved in ethyl acetate (150 mL) and washed with water, brine and dried over sodium sulfate. The solvent was concentrated under vacuo to yield Intermediate 144 as a white solid (1.5 g).

MS(ES): 365 (M+2) for C₁₆H₁₉BrN₄O.

¹H-NMR (400 MHz, DMSO-d₆): δ 2.21 (s, 6H), 3.57-3.59 (m, 4H), 3.72-3.73 (m, 4H), 6.57 (s, 1H), 7.32 (s, 2H), 8.21 (d, J=1.2 Hz, 1H), 9.35 (s, 1H).

### Intermediate 145: 3-[3-(2-Chloro-4-morpholin-4-yl-pyrimidin-5-yl)-phenyl]-acrylic acid ethyl ester

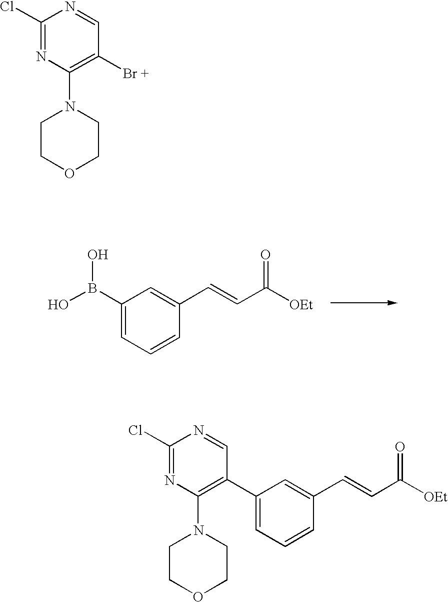

A suspension of 4-(5-Bromo-2-chloro-pyrimidin-4-yl)-morpholine Intermediate 136 (3.5 mmol, 1 g), ethyl boronocinnamate (3.95 mmol, 0.87 g), [1,1′-bis(diphenylphosphino)ferrocene]dichloropalladium(II) complex with CH₂Cl₂ (0.35 mmol, 285 mg) and sodium carbonate (3.5 mmol, 370 mg) in acetonitrile/water (20 mL:5 mL) was degassed and heated to 90° C. for 15-20 min under inert atmosphere. The solvent was removed in vacuo and the crude mixture was taken in CHCl₃ (30 mL). It was then washed with water and brine. The organic layer was separated, dried over sodium sulfate, filtered and concentrated. The crude material was purified by 60-120 mesh silica gel column chromatography using Hexane:Ethylacetate (91:9) to get 3-[3-(2-Chloro-4-morpholin-4-yl-pyrimidin-5-yl)-phenyl]-acrylic acid ethyl ester Intermediate 145 (0.63 g). MS (ES): 374 (M+1) for C₁₉H₂₀ClN₃O₃.

¹H-NMR (300 MHz, DMSO-d₆): δ 1.25 (t, J=7.05 Hz, 3H), 3.25-3.32 (m, 4H), 3.49-3.50 (m, 4H), 4.19 (q, J=7.05 Hz, 2H), 6.74 (d, J=16.05 Hz, 1H), 7.50-7.59 (m, 2H), 7.69 (d, J=16.14 Hz, 1H), 7.73 (br s, 1H), 7.83 (s, 1H), 8.11 (s, 1H).

### Intermediate 146: 5-bromo-N-(3-chloro-4-fluorophenyl)-4-morpholinopyrimidin-2-amine

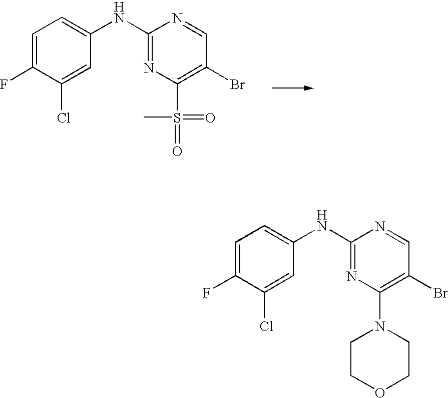

5-Bromo-2[N-(3-chloro-4-fluorophenyl)]-4-(methylsulfonyl)pyrimidin-2-amine Intermediate 69 (15.7 mmol, 6 g) was suspended in NMP (30 mL) and treated with N,N-diisopropylethylamine (23.6 mmol, 4 mL) and morpholine (18.9 mmol, 1.64 g) in a 100 mL round-bottomed flask. The reaction was refluxed at 90° C. for 45 min. The reaction mixture was added to water and stirred for 15 min The precipitated solid was filtered and washed successively with water, diethyl ether and hexanes and dried to afford 4.96 g of Intermediate 146 (12.56 mmol, 80%). MS(ES): 387 (M) and 389 (M+2) for C₁₄H₁₃BrClFN₄O.

¹H NMR 400 MHz DMSO-d₆: δ 3.57-3.59 (m, 4H), 3.71-3.73 (m, 4H), 7.32 (t, J=9.08 Hz, 1H), 7.55 (ddd, J=9.00, 4.18, 2.68 Hz, 1H), 8.04 (dd, J=6.86, 2.64 Hz, 1H), 8.26 (d, J=1.20 Hz, 1H), 9.71 (s, 1H).

### Intermediate 147: 5-bromo-N-(4-fluoro-3-nitrophenyl)-4-methoxypyrimidin-2-amine

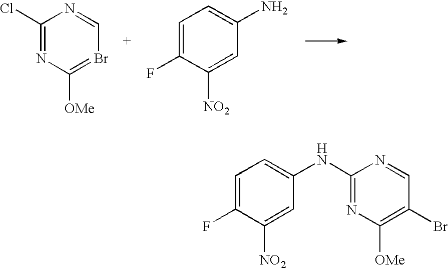

To a stirred solution of 5-bromo-2-chloro-4-methoxypyrimidine (8.96 mmol, 2 g) and (4-fluoro-3-nitrophenyl)amine (9.41 mmol, 1.47 g) in butanol (40 mL), 4 M HCl in 1,4-dioxane (2.5 mL) was added dropwise. The resulting solution was refluxed at 100° C. with constant stirring. The solvent was removed in vacuo and basified with 10% NaHCO₃ solution. It was further extracted with EtOAc (2×100 mL). The combined organic layer was washed with water and brine, dried over sodium sulfate and concentrated under vacuum to yield 5-bromo-N-(4-fluoro-3-nitrophenyl)-4-methoxypyrimidin-2-amine Intermediate 147 as a white solid (2 g).

MS(ES): 343 (M) and 345 (M+2) for C₁₁H₈BrFN₄O₃.

¹H-NMR (400 MHz, DMSO-d₆: δ 4.04 (s, 3H), 7.53 (dd, J=9.28, 11.02 Hz, 1H), 7.92-7.96 (m, 1H), 8.45 (s, 1H), 8.79 (dd, J=2.56, 6.76 Hz, 1H), 10.24 (br s, 1H).

### Intermediate 148: 5-bromo-2-[(4-fluoro-3-nitrophenyl)amino]pyrimidin-4-ol

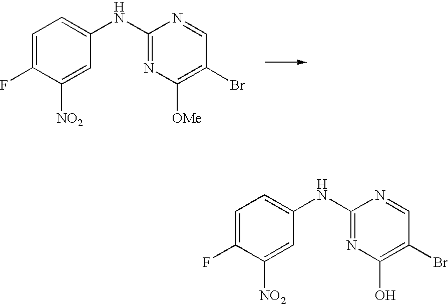

A mixture of 5-bromo-N-(4-fluoro-3-nitrophenyl)-4-methoxypyrimidin-2-amine Intermediate 147 (5.82 mmol, 2 g) and HBr in AcOH (12 mL) and aq. HBr (6 mL) was heated at 100° C. for 3 h; The reaction mixture was cooled to RT, extracted with ethyl acetate (2×100mL), washed with brine, dried over Na₂SO₄ and concentrated to yield 1.3 g of 5-bromo-2-[(4-fluoro-3-nitrophenyl)amino]pyrimidin-4-ol Intermediate 148 as an off-white solid.

MS(ES): 329 (M) and 331 (M+2) for C₁₀H₆BrFN₄O₃.

¹H-NMR (400 MHz, DMSO-d6): δ 7.55 (dd, J=9.16, 11.04 Hz, 1H), 7.87-7.89 (m, 1H), 8.12 (s, 1H), 8.47 (dd, J=2.80, 6.74 Hz, 1H), 9.39 (br s, 1H), 11.8 (br s, 1H).

### Intermediate 149: 5-bromo-4-chloro-N-(4-fluoro-3-nitrophenyl)pyrimidin-2-amine

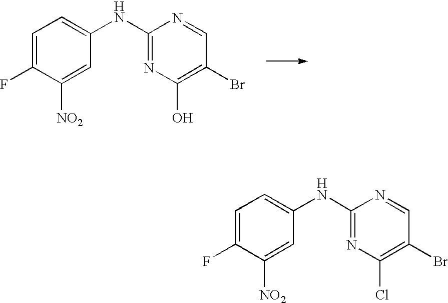

A solution of 5-bromo-2-[(4-fluoro-3-nitrophenyl)amino]pyrimidin-4-ol Intermediate 148 (3.95 mmol, 1.3 g) in phosphorus oxychloride (6 mL) was heated to reflux for 45 minutes, cooled to RT, poured carefully onto a mixture of ice (100 mL) and sat. NaHCO₃ (20 mL) with constant stirring. It was further extracted with EtOAc (2×150 mL). The combined organic layers were washed with water and brine, dried over sodium sulfate and concentrated under vacuum to yield 5-bromo-4-chloro-N-(4-fluoro-3-nitrophenyl)pyrimidin-2-amine Intermediate 149 (1 g).

MS(ES): 347 (M) and 349 (M+2) for C₁₀H₅BrClFN₄O₂.

¹H-NMR (300 MHz, DMSO-d₆): δ 7.57 (dd, J=9.24, 11.05 Hz, 1H), 7.93-7.98 (m, 1H), 8.57 (dd, J=2.73, 6.73 Hz, 1H), 8.77 (s, 1H), 10.63 (br s, 1H).

### Intermediate 150: 5-bromo-N-(4-fluoro-3-nitrophenyl)-4-morpholin-4-ylpyrimidin-2-amine

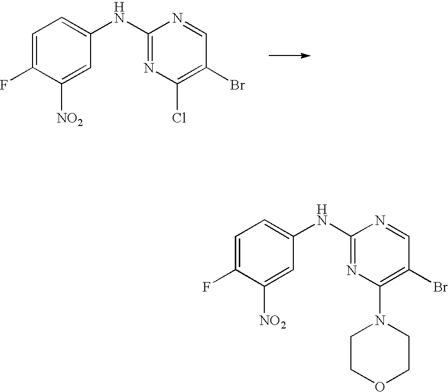

A mixture of 5-bromo-4-chloro-N-(4-fluoro-3-nitrophenyl)pyrimidin-2-amine Intermediate 149 (2.88 mmol, 1 g) and morpholine (3.45 mmol, 0.3 g) was stirred at room temperature for 18 h. Then the reaction mixture was concentrated under vacuum to yield white solid. The solid was dissolved in EtOAc (100 mL) and washed with water, brine and dried over sodium sulfate. The solvent was concentrated under vacuum to yield 5-bromo-N-(4-fluoro-3-nitrophenyl)-4-morpholin-4-ylpyrimidin-2-amine Intermediate 150 as a white solid (1 g).

MS(ES): 398 (M) and 400 (M+2) for C₁₄H₁₃BrFN₅O₃.

400 MHz, DMSO-d₆: δ 3.62-3.63 (m, 4H), 3.71-3.72 (m, 4H), 7.50 (t, J=10.24 Hz, 1H), 7.83-7.86 (m, 1H), 8.30 (s, 1H), 8.81 (d, J=6.24 Hz, 1H), 10.04 (s, 1H).

### Intermediate 151: 4-(5-Bromo-2-chloro-pyrimidin-4-yl)-morpholine

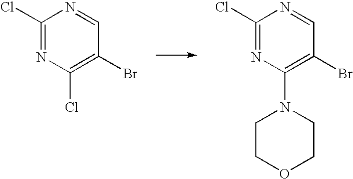

To a stirred solution of 2,4-dichloro-5-bromopyrimidine (22 mmol, 5 g) in dioxane (50 mL), at room temperature under nitrogen atmosphere was added morpholine (29.2 mmol, 2.54 g). Further dilution with dioxane (50 mL) became necessary as the reaction progressed. The reaction mixture was stirred overnight at room temperature, then diluted with ethyl acetate (50 mL) and white solid formed was removed by filtration. The filtrate was concentrated and the resulting residue was purified by column chromatography (using 60-120 mesh silica gel and 4% of ethylacetate in petroleum ether) to give 4-(5-Bromo-2-chloro-pyrimidin-4-yl)-morpholine Intermediate 151 (4.7 g).

MS (ES): 278 (M) and 280 (M+2) for C₈H₉BrClN₃O.

¹H-NMR (300 MHz, CDCl₃): δ 3.79 (s, 8H), 8.27 (s, 1H).

### Intermediate 152: 5-(2-Chloro-4-morpholin-4-yl-pyrimidin-5-yl)-nicotinic acid ethyl ester

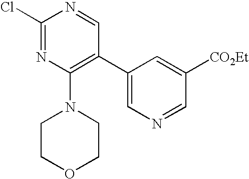

A suspension of 4-(5-Bromo-2-chloro-pyrimidin-4-yl)-morpholine Intermediate 151 (3.5 mmol, 1 g), 5-(4,4,5,5-Tetramethyl-[1,3,2]dioxaborolan-2-yl)-nicotinic acid ethyl ester (3.6 mmol, 1.01 g), [1,1′-bis(diphenylphosphino)ferrocene]dichloropalladium(II)complex with CH₂Cl₂ (10 mol %, 0.35 mmol, 0.285 g) and sodium carbonate (3.5 mmol, 0.370 g) were taken in acetonitrile/water (20 mL:5 mL), degassed and heated to 90° C. for 15-20 min under inert atmosphere. The solvent was removed in vacuo and the crude mixture was taken in CHCl₃ (30 mL). It was then washed with water and brine. The organic layer was separated, dried over sodium sulfate, filtered and concentrated. The crude material was purified by silica gel column chromatography. The product eluted with Hexane: Ethyl Acetate (3:1) eluent mixture. The title compound Intermediate 152 was obtained (0.69 g). MS (ES) : 349 (M+1) for C₁₆H₁₇ClN₄O₃.

¹H-NMR (300 MHz, DMSO-d₆): δ 0.81 (t, J=6.54 Hz, 3H), 3.24-3.25 (m, 4H), 3.49-3.50 (m, 4H), 4.37 (q, J=7.05 Hz, 2H), 8.20 (s, 1H), 8.37 (s, 1H), 8.91 (d, J=1.98 Hz, 1H), 9.07 (d, J=1.80 Hz, 1H).

### Intermediate 153: 3-[3-(2-Chloro-4-morpholin-4-yl-pyrimidin-5-yl)-phenyl]-acrylic acid ethyl ester

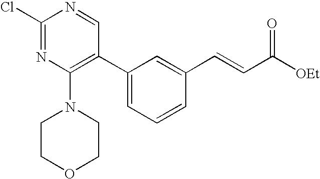

A suspension of 4-(5-Bromo-2-chloro-pyrimidin-4-yl)-morpholine Intermediate 151_(3.5 mmol, 1 g), ethyl boronocinnamate (3.95 mmol, 0.87 g), [1,1′-bis(diphenyl-phosphino)ferrocene]dichloropalladium(II)complex with CH₂Cl₂ (10 mol %, 0.35 mmol, 0.285 g) and sodium carbonate (3.5 mmol, 0.370 g) in acetonitrile/water (20 mL:5 mL) was degassed and heated to 90° C. for 15-20 min under inert atmosphere. Completion of the reaction was monitored by TLC. The solvent was removed in vacuo and the crude mixture was taken in CHCl₃ (30 mL). It was then washed with water and brine. The organic layer was separated, dried over sodium sulfate, filtered and concentrated. The crude material was purified by silica gel column chromatography. The product eluted with Hexane:Ethyl Acetate (91:9) eluent mixture. The title compound Intermediate 153 was obtained (0.63 g).

MS (ES) : 374 (M+1) for C₁₉H₂₀ClN₃O₃.

¹H-NMR (300 MHz, DMSO-d₆): δ 1.25 (t, J=7.05 Hz, 3H), 3.25-3.32 (m, 4H), 3.49-3.50 (m, 4H), 4.19 (q, J=7.05 Hz, 2H), 6.74 (d, J=16.05 Hz, 1H), 7.50-7.59 (m, 2H), 7.69 (d, J=16.14 Hz, 1H), 7.73 (br s, 1H), 7.83 (s, 1H), 8.11 (s, 1H).

### Intermediate 154: methyl 6-bromo-4-oxo-4H-chromene-3-carboxylate

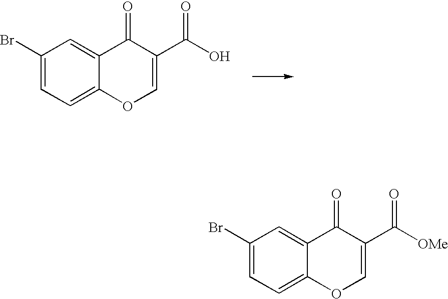

To a solution of 6-bromo-4-oxo-4H-chromene-3-carboxylic acid (3.7 mmol, 1 g) in methanol (15 mL) was added thionyl chloride (5 mL) slowly at 0° C. The reaction mixture was then refluxed at 70° C. for 2 h and then cooled to room temperature. Methanol was removed in vacuo and the residue was poured on to ice water (25 mL), extracted with ethyl acetate (2×10 mL). The combined organic extracts were washed with 10% sodium bicarbonate solution (2×10 mL), brine, dried over anhydrous sodium sulphate, filtered and concentrated to get methyl 6-bromo-4-oxo-4H-chromene-3-carboxylate Intermediate 154 as yellow solid (900 mg).

MS(ES): 283 (M) and 285.2 (M+2) for C₁₁H₇BrO₄.

¹H-NMR(400 MHz, DMSO-d₆): δ 3.80 (s, 3H), 7.74 (d, J=8.80 Hz, 1H), 8.03 (dd, J=2.80, 9.00 Hz, 1H), 8.16 (d, J=2.40 Hz, 1H), 9.01 (s, 1H).

### Intermediate 155: methyl 4-oxo-6-(4,4,5,5-tetramethyl-1,3,2-dioxaborolan-2-yl)-4H-chromene-3-carboxylate and [3-(methoxycarbonyl)-4-oxo-4H-chromen-6-yl]boronic acid

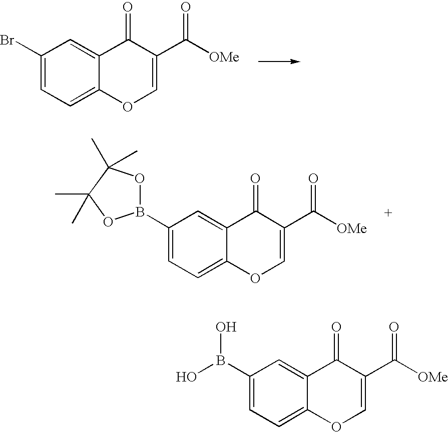

A mixture of methyl 6-bromo-4-oxo-4H-chromene-3-carboxylate Intermediate 154 (1.41 mmol, 400 mg), bis(pinacolato)diboron (3.94 mmol, 1 g), 2-Dicyclohexylphosphino-2′,4′,6′-triisopropylbiphenyl (20 mol %, 0.28 mmol, 135 mg), palladium acetate (4 mol %, 0.06 mmol, 16 mg) and triethylamine (5.6 mmol, 570 mg) in dioxane (20 mL) was degassed and then refluxed at 100° C. for 20 min under nitrogen atmosphere. The solvent was removed in vacuo, residue diluted with dichloromethane (20 mL), washed with water (2×20 mL), brine. The organic layer was dried over anhydrous sodium sulphate, filtered and concentrated and the resulting residue was then recrystallized with dichloromethane-hexane to get 300 mg of a mixture of methyl 4-oxo-6-(4,4,5,5-tetramethyl-1,3,2-dioxaborolan-2-yl)-4H-chromene-3-carboxylate and [3-(methoxycarbonyl)-4-oxo-4H-chromen-6-yl]boronic acid, Intermediate 155.

MS(ES): 249.2 (M+1) for C₁₁H₉BO₆. LC=58.64% of boronic acid and 331.2 (M+1) for C₁₇H₁₉BO₆ LC=36.05% for pinacol boronate.

### Intermediate 156: 3-Chloro-4-fluoro-phenyl)-(4-methanesulfonyl-2′-methoxy-[5,5′]bipyrimidinyl-2-yl)-amine

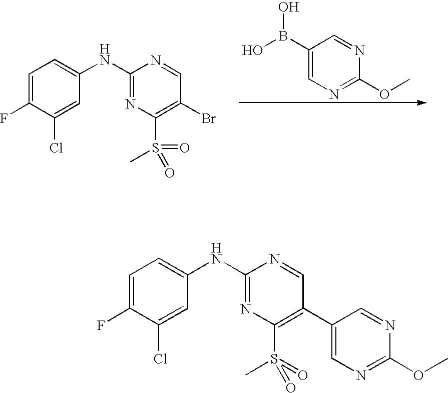

A suspension of sultone Intermediate 69 (mmol, 3 g), methoxypyrimidineboronic acid (8.6 mmol, 1.38 g), dichlorobis(triphenyl-phosphine)palladium(II) (0.6 mmol, 437 mg) and sodium carbonate (7.8 mmol, 826 mg) in dioxane (20 mL)-water (5 mL) was degassed and refluxed at 100° C. for 15-20 minutes. Dioxane was removed under vacuum and the residue taken in chloroform (50 mL), washed with water (50 mL) and brine. The layers were separated and the organic layer was dried over sodium sulphate, filtered and concentrated.

The resulting residue was then purified by column chromatography using 230-400 mesh silica gel and 2% of methanol in chloroform as eluent. The solid thus obtained was stirred with acetonitrile for 15 minutes. The solid was filtered and dried under vacuum to yield the title compound as solid (1.4 g). MS(ES): 410.2 (M+1) for C₁₆H₁₃ClFN₅O₃S.

¹H-NMR(300 MHz, DMSO-d₆): δ 3.39 (s, 3H), 3.96 (s, 3H), 7.43 (t, J=9.09 Hz, 1H), 7.60-7.65 (m, 1H), 8.00 (dd, J=2.40, 6.61 Hz, 1H), 8.67 (s, 2H), 8.81 (s, 1H), 10.57 (s, 1H).

### Intermediate 157: methyl 4-{2-[(3-chloro-4-fluorophenyl)amino]-4-(methylsulfonyl)pyrimidin-5-yl}pyridine-2-carboxylate

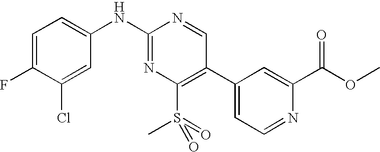

A mixture of methyl 4-(4,4,5,5-tetramethyl-1,3,2-dioxaborolan-2-yl)pyridine-2-carboxylate (1.1 eq, 0.289 mmol, 0.076 g) and 2,8,9-Triisobutyl-2,5,8,9-tetraaza-1-phosphabicyclo[3.3.3]undecane (0.1 eq, 0.024 mmol, 8.1 mg) was stirred for 20 min under N₂ in acetonitrile (3 mL). [Flask I]

In a separate flask, 5-bromo-N-(3-chloro-4-fluorophenyl)-4-(methylsulfonyl)pyrimidin-2-amine, (Intermediate 69) (1 eq, 0.263 mmol, 100 mg) was mixed with [1,1′-bis(diphenylphosphino)ferrocene]dichloropalladium(II)complex with CH₂Cl₂ (10 mol %, 21.5 mg) and stirred for 10 min under N₂ in acetonitrile (3 mL). [Flask II]

The contents of [Flask I] were quickly transferred to [Flask II]. Water (1.5 mL) was added and the mixture was heated at 90° C. for 20-25 minutes then concentrated in vacuo. The resulting slurry was taken in EtOAc and washed with water and brine. The organic layer was separated, dried over sodium sulfate, filtered and concentrated.

The reaction was repeated on the same scale using the above procedure. The crude materials from both batches were combined and purified by silica gel column chromatography (60-120 mesh) using ethyl acetate/hexanes (2:3 to 3:2) as an eluent. The title compound was thus obtained (0.09 g). MS(ES): 437 (M+1) for C₁₈H₁₄ClFN₄O₄S.

400 MHz, DMSO-d₆: δ 3.38 (s, 3H), 3.90 (s, 3H), 7.44 (t, J=9.08 Hz, 1H), 7.64 (ddd, J=2.72, 4.10, 9.03 Hz, 1H), 7.75 (dd, J=1.76, 4.94 Hz, 1H), 8.00 (dd, J=2.40, 6.66 Hz, 1H), 8.19 (d, J=1.12 Hz, 1H), 8.78 (d, J=4.96 Hz, 1H), 8.82 (s, 1H), 10.63 (s, 1H).

### Intermediate 158: 2-(3-chloro-4-fluorophenylamino)-4-(3-methoxypropylamino)pyrimidine-5-carbonitrile

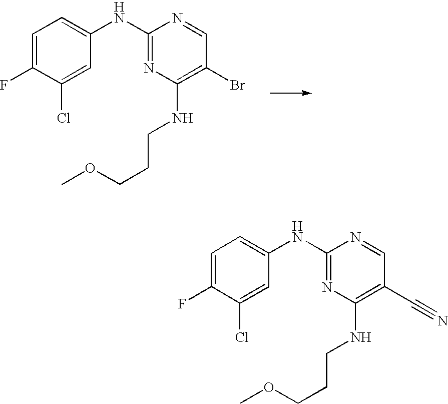

To a stirred suspension of 5-bromo-N²-(3-chloro-4-fluorophenyl)-N⁴-(3-methoxypropyl)pyrimidine-2,4-diamine, Intermediate 119 (7.7 mmol, 3 g, 1 eq), zinc acetate (0.306 mmol, 0.056 g, 0.04 eq), zinc (1.9 mmol, 0.124 g, 2.5 eq), tris(dibenzylideneacetone)-dipalladium (0) (0.15 mmol, 0.135 g, 0.02 eq) and 1,1′-bis(diphenylphosphino)ferrocene (0.3 mmol, 0.168 g, 0.04 eq) in degassed DMF (30 mL) was added zinc cyanide (0.580 g, 4.9 mmol). The vessel was evacuated and backfilled with argon; this process was repeated once. The mixture was placed under an atmosphere of nitrogen, stirred, and heated to 100° C. for 3 hours. The stirring was continued while the mixture was allowed to cool to room temperature; the mixture was then diluted with small volumes of water. The dark solution became cloudy with addition of water. Larger volumes were added until a maximum of precipitation was reached. The stirring was continued for ten minutes. The solid was collected and washed with water to give the title compound (2.1 g). MS(ES): 336 (M+1) for C₁₅H₁₅ClFN₅O.

300 MHz, DMSO-d₆: δ 1.76-1.85 (m, 2H), 3.21 (s, 3H), 3.38 (t, J=6.15 Hz, 2H), 3.41-3.47 (m, 2H), 7.33 (t, J=9.09 Hz, 1H), 7.57-7.62 (m, 1H), 7.87 (br s, 1H), 8.09-8.12 (m, 1H), 8.36 (s, 1H), 9.99 (br s, 1H).

Intermediate 159: 2-[(3-chloro-4-fluorophenyl)amino]-4-[(3-methoxypropyl)amino]pyrimidine-5-carbothioamide

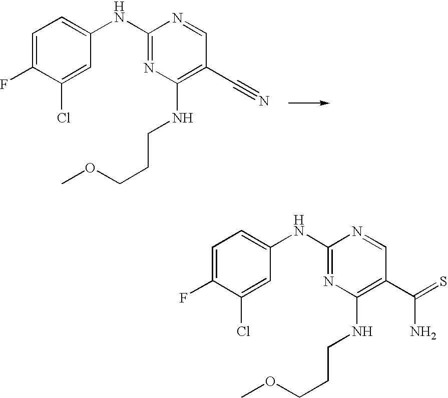

To 2-(3-chloro-4-fluorophenylamino)-4-(3-methoxypropylamino)pyrimidine-5-carbonitrile, Intermediate 158 (6.8 mmol, 2.3 g) in 5 mL DMF, ammonium sulfide, (20% aq solution, 14 mL, 41 mmol) was added and heated at 70° C. in a sealed tube for 4 h. The reaction mixture was quenched with water and the solid was filtered to give the title compound (2 g). MS(ES): 368 (M−1) for C₁₅H₁₇ClFN₅OS.

400 MHz, DMSO-d₆: δ 1.80-1.87 (m, 2H), 3.32 (s, 3H), 3.43 (t, J=6.12 Hz, 2H), 3.52 (q, J=6.56 Hz, 2H), 7.32 (t, J=9.08 Hz, 1H), 7.59-7.63 (m, 1H), 8.21 (dd, J=2.48, 6.84 Hz, 1H), 8.35 (s, 1H), 9.26 (br s, 1H), 9.37 (br s, 1H), 9.63 (br s, 1H), 9.79 (br s, 1H).

### Intermediate 160: 2-[(3-chloro-4-fluorophenyl)amino]-4-(morpholin-4-yl)pyrimidine-5-carboxylic acid

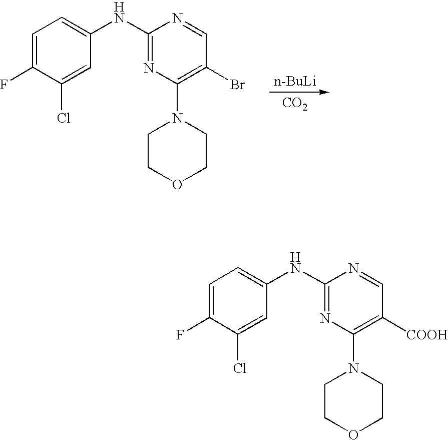

To 5-bromo-N-(3-chloro-4-fluorophenyl)-4-(morpholin-4-yl)pyrimidin-2-amine Intermediate 146, 0.51 mmol, 0.2 g) in dry THF at −78° C. under nitrogen atmosphere, n-BuLi (1.6 M in Hexanes, 1.02 mmol, 0.64 mL) was added dropwise and stirred for 20 min Carbon dioxide gas was passed into the reaction mixture for 10 min with constant stirring. The solvent was evaporated and the residue was dissolved in water followed by washing with ethyl acetate. The aqueous layer was acidified with 1 N HCl and the precipitate formed was filtered and dried to yield 45 mg of 2-[(3-chloro-4-fluorophenyl)amino]-4-(morpholin-4-yl)pyrimidine-5-carboxylic acid.

| | | Mass spectrum and ¹H | |
|---|---|---|---|
| Compound | Structure | NMR | SM |
| Intermediate 160 | 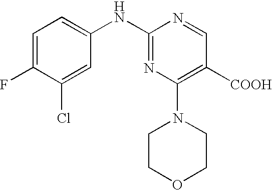 2-[(3-chloro-4-fluorophenyl)amino]-4- (morpholin-4-yl)pyrimidine-5- carboxylic acid | MS(ES): 353 (M + 1) for C₁₅H₁₄ClFN₄O₃. 400 MHz, DMSO-d₆: δ 3.51-3.52 (m, 4H), 3.66- 3.68 (m, 4H), 7.33 (t, J = 9.12 Hz, 1H), 7.56-7.60 (m, 1H), 8.03 (dd, J = 2.60, 6.84 Hz, 1H), 8.54 (s, 1H), 9.86 (br s, 1H), 12.58 (br s, 1H). | Intermediate 146 |

### Intermediate 161: N-(2-aminophenyl)-2-[(3-chloro-4-fluorophenyl)amino]-4-(morpholin-4-yl)pyrimidine-5-carboxamide

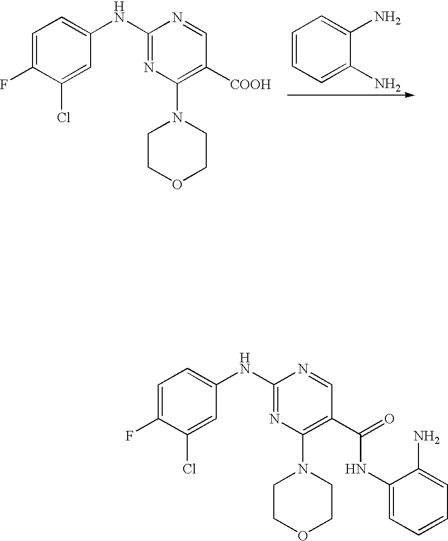

To a mixture of 2-[(3-chloro-4-fluorophenyl)amino]-4-(morpholin-4-yl)pyrimidine-5-carboxylic acid (Intermediate 160, 0.85 mmol, 0.3 g) and (Benzotriazol-1-yloxy)tris(dimethylamino)phosphonium hexafluorophosphate (1 mmol, 442 mg) in DMF at 0° C., Et₃N (2.14 mmol, 216 mg, 0.3 mL) was added and stirred for 5 min 1,2-phenylenediamine (1 mmol, 110 mg) in DMF was added drop wise to the reaction mixture and stirred at RT for 4-5 h. Water was added to the mixture and stirred overnight. The solid thus obtained was filtered and purified by column chromatography using methanol: chloroform (3:97) to yield the title compound (70 mg).

| | | Mass spectrum and ¹H | |
|---|---|---|---|
| Compound | Structure | NMR | SM |
| Intermediate 161 | 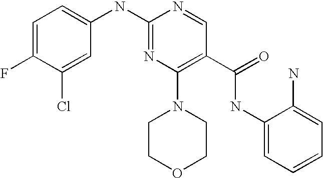 N-(2-aminophenyl)-2-[(3-chloro-4- fluorophenyl)amino]-4-(morpholin-4- yl)pyrimidine-5-carboxamide | MS(ES): 443 (M + 1) for C₂₁H₂₀ClFN₆O₂. 400 MHz, DMSO-d₆: δ 3.55- 3.56 (m, 4H), 3.65-3.67 (m, 4H), 4.95 (br s, 2H), 6.57 (t, J = 7.60 Hz, 1H), 6.74 (d, J = 7.96 Hz, 1H), 6.92 (t, J = 7.36 Hz, 1H), 7.29 (d, J = 7.68 Hz, 1H), 7.33 (t, J = 9.12 Hz, 1H), 7.60 (dd, J = 2.56, 8.38 Hz, 1H), 8.08 (d, J = 4.52 Hz, 1H), 8.35 (s, 1H), 9.52 (s, 1H), 9.71 (s, 1H). | Intermediate 160 2-[(3-chloro- 4- fluorophenyl) amino]-4- (morpholin-4- yl)pyrimidine- 5-carboxylic acid |

### Intermediate 162: N-methoxy-N,5-dimethylisoxazole-4-carboxamide

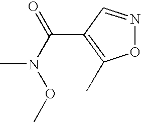

To a mixture of 5-methylisoxazole-4-carboxylic acid (3.5 mmol, 500 mg), triethylamine (11.8 mmol, 1.19 g, 1.6 mL) and N,O-Dimethylhydroxylamine hydrochloride (5.1 mmol, 498 mg) in DCM, was added T3P (50% in EtOAc, 3.3 mL, 6 mmol) slowly at 0° C. The reaction mixture was slowly raised to room temperature and stirred for 12 h. Reaction mixture was then diluted with dichloromethane (12 mL) and the dichloromethane layer was successively with water (2×50 mL), 10% aq sodium bicarbonate solution (50 mL) and brine. The organic layer was dried over sodium sulphate, filtered and concentrated. The residue was further purified using column chromatography (60-120 mesh; product eluted at 15% ethyl acetate/hexanes) to yield 480 mg of the title compound.

| | | Mass spectrum and ¹H | |
|---|---|---|---|
| Compound | Structure | NMR | SM |
| Intermediate 162 | 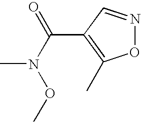 N-methoxy-N,5- dimethylisoxazole- 4-carboxamide | MS(ES): 171 (M + 1) for C₇H₁₀N₂O₃. 300 MHz, CDCl₃: δ 2.71 (s, 3H), 3.34 (s, 3H), 3.69 (s, 3H), 8.54 (s, 1H). | 5- methylisoxazole- 4-carboxylic acid |

### Intermediate 163: 1-(5-methylisoxazol-4yl)-ethanone

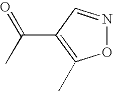

To 480 mg of N-methoxy-N,5-dimethylisoxazole-4-carboxamide (Intermediate 162, 2.8 mmol) taken in dry ether at 0° C., was added drop wise a solution of methyl magnesium bromide (3 M in ether) (2.8 mL, 8.4 mmol, 3 eq). The contents of the flask were then slowly brought to room temperature and stirred for 3 h. The reaction was quenched with saturated ammonium chloride (2×20 mL) and extracted with Et₂O. The organic layer was dried over sodium sulphate, filtered and concentrated to yield 200 mg of the title compound.

| | | Mass spectrum and ¹H | |
|---|---|---|---|
| Compound | Structure | NMR | SM |
| Intermediate 163 | 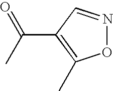 1-(5- methylisoxazol-4- yl)ethanone | MS(ES): 126 (M + 1) (84% purity) for C₆H₇NO₂ 300 MHz, CDCl₃: δ 2.47 (s, 3H), 2.73 (d, J = 0.45 Hz, 3H), 8.48 (d, J = 0.48 Hz, 1H). | Intermediate 162 N-methoxy-N,5- dimethyliso- xazole-4- carboxamide |

### Intermediate 164: 2-bromo-1-(5-methylisoxazol-4-yl)ethanone

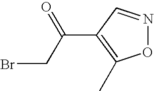

To 340 mg of 1-(5-methylisoxazol-4-yl)ethanone (Intermediate 163, 2 7 mmol) taken in dry THF at room temperature, was added phenyltrimethylammonium tribromide (3 mmol, 1.12 g) and refluxed for 2 h. The reaction was quenched with water (2×20 mL) and extracted with ethyl acetate. The organic layer was dried over sodium sulphate, filtered and concentrated. The resulting residue was purified by silica gel column chromatography (60-120 mesh; product eluted at 15% ethyl acetate/hexanes) to afford 100 mg of the title compound.

| | | Mass spectrum and ¹H | |
|---|---|---|---|
| Compound | Structure | NMR | SM |
| Intermediate 164 | 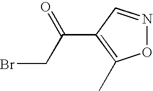 2-bromo-1-(5- methylisoxazol-4- yl)ethanone | MS(GC): 110 (100%), 123, 203 (M) and 205 (M + 2) for C₆H₆BrNO₂. 300 MHz, CDCl₃: δ 2.76 (s, 3H), 4.14 (s, 2H), 8.58 (s, 1H). | Intermediate 163 1-(5- methylisoxazol- 4-yl)ethanone |

### Intermediate 165: N-methoxy-N,1-dimethyl-1H-imidazole-5-carboxamide

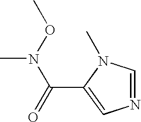

To a mixture of 1-methyl-1H-imidazole-5-carboxylic acid (7.93 mmol, 1 g), triethylamine (19.83 mmol, 2.77 mL) and N,O-Dimethylhydroxylamine hydrochloride (9.52 mmol, 928 mg) in DCM, was added T3P (50% in EtOAc, 11.89 mol, 7.57 mL) slowly at 0° C. The reaction mixture was slowly raised to room temperature and stirred overnight. Reaction mixture was then diluted with dichloromethane (12 mL), washed the dichloromethane solution successively with water (2×50 mL), 10% aq sodium bicarbonate solution (50 mL) and brine. The organic layer was dried over sodium sulphate, filtered and concentrated. The residue was further purified using column chromatography (hexane-ethyl acetate as the eluent) to yield the title compound (1.13 g).

| | | Mass spectrum and ¹H | |
|---|---|---|---|
| Compound | Structure | NMR | SM |
| Intermediate 165 | 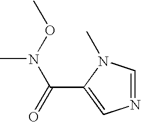 N-methoxy-N,1- dimethyl-1H- imidazole-5- carboxamide | MS(ES): 170 (M + 1) for C₇H₁₁N₃O₂. 400 MHz, MeOD: δ 3.34 (s, 3H), 3.74 (s, 3H), 3.93 (br s, 3H), 7.74 (br s, 1H), 7.91 (s, 1H). | 1-methyl-1H- imidazole-5- carboxylic acid |

### Intermediate 166: 1-(1-methyl-1H-imidazol-5-yl)ethanone

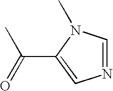

To 1.3 g of N-methoxy-N,1-dimethyl-1H-imidazole-5-carboxamide (Intermediate 165, 7.68 mmol) taken in dry ether at 0° C., was added drop wise a solution of methyl magnesium bromide (3 M in ether) (7.69 mL, 23 mmol). The contents of the flask were then slowly brought to room temperature and stirred for 3 h. The reaction was quenched with 1.5 N HCl (2×20 mL) and extracted with Et₂O. The organic layer was dried over sodium sulphate, filtered and concentrated to yield 800 mg of the title compound.

| | | Mass spectrum and ¹H | |
|---|---|---|---|
| Compound | Structure | NMR | SM |
| Intermediate 166 | 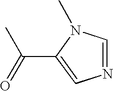 1-(1-methyl-1H- imidazol-5- yl)ethanone | MS(GC): 125 (M + 1), 124 (M), 109 (M − 15), 81 (M − 43) for C₆H₈N₂O. 400 MHz, CDCl₃: δ 2.46 (s, 3H), 3.91 (s, 3H), 7.55 (br s, 1H), 7.76 (br s, 1H). | Intermediate 165 N-methoxy-N,1- dimethyl-1H- imidazole-5- carboxamide |

### Intermediate 167: 2-bromo-1-(1-methyl-1H-imidazol-5-yl)ethanone and 2,2-dibromo-1-(1-methyl-1H-imidazol-5-yl)ethanone

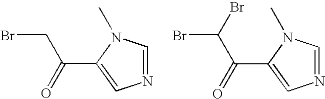

1-(1-methyl-1H-imidazol-5-yl)ethanone (2.89 mmol, 0.36 g) was dissolved in 5 mL of 48% hydrogen bromide. To the stirred solution at 25° C. was added over a 5 min period bromine (3.19 mmol, 0.163 mL, 0.51 g) dissolved in 5 mL of 48% hydrogen bromide. The reaction mixture was heated at 70° C. for 2.5 h and then concentrated in vacuo to a dark oil. A mixture of isopropyl alcohol/diethyl ether was added, and trituration of the oil gave a solid. This was collected by filtration and washed with diethyl ether to give 0.28 g of a mixture of 2-bromo-1-(1-methyl-1H-imidazol-5-yl)ethanone and 2,2-dibromo-1-(1 -methyl-1H-imidazol-5-yl)ethanone.

| | | Mass spectrum and ¹H | |
|---|---|---|---|
| Compound | Structure | NMR | SM |
| Intermediate 167 | 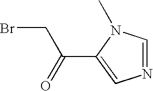 + 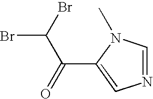 2-bromo-1-(1- methyl-1H- imidazol-5- yl)ethanone and 2,2-dibromo-1-(1- methyl-1H- imidazol-5- yl)ethanone | Obtained as a mixture of monobromo and dibromo ketones. MS(GC) for the monobromoketone: 202 and 204 for C₆H₇BrN₂O. 1H NMR for the dibromoketone 400 MHz, DMSO-d₆: δ 3.98 (s, 3H), 7.56 (s, 1H), 8.77 (s, 1H), 9.17 (s, 1H). | Intermediate 166 (1-(1-methyl-1- 1H-imidazol-5- yl)ethanone |

### Intermediate 168: [1-ethoxy-1,3-dioxo-3-(pyridin-2-yl)propan-2-ylidene]diazenium

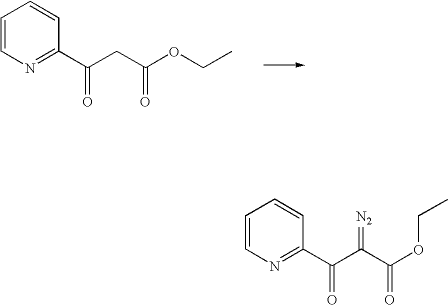

To a mixture of ethyl 3-oxo-3-(pyridin-2-yl)propanoate (0.52 mmol, 100 mg), p-Acetamidobenzene sulfonyl azide (0.52 mmol, 114 mg) in dry acetonitrile (5 mL) was added triethyl amine (1.56 mmol, 157 mg) slowly at 0° C. The reaction mixture was slowly raised to room temperature and stirred for 2-3 h. The solvent was removed in vacuo and the resulting residue was stirred with petroleum ether: diethyl ether (1:1) mixture for 0.5 h and filtered. The filtrate was concentrated to yield 100 mg of the title compound as brownish liquid, which was used in the next step without further purification.

MS(ES): 220 (M+1) for C₁₀H₉N₃O₃.

### Intermediate 169: 1-methyl-1H-pyrazole-3-carbonyl chloride

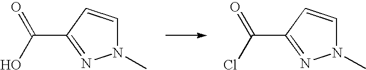

To a suspension of 1-methyl-1H-pyrazole-3-carboxylic acid (0.3 g, 2.38 mmol) in dichloromethane (10 mL) was added oxalyl chloride (0.3 mL, 3.6 mmol) dropwise at 0° C. The resulting mixture was stirred at room temperature for 1 h and then heated to 70° C. for 2 h. After cooling to room temperature, the excess oxalyl chloride and solvent was removed by rotary evaporator (with water bath temperature below 40° C.) to afford the title compound as a gum, which was used in the next step without further purification.

### Intermediate 170: ethyl 3-(1-methyl-1H-pyrazol-3-yl)-3-oxopropanoate

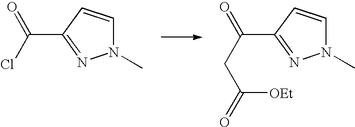

0.53 g of ethyl hydrogen malonate (4.05 mmol) was taken in anhydrous tetrahydrofuran (10 mL) in a 2-necked 100 mL round bottom flask equipped with a nitrogen inlet. After cooling to −78° C., n-butyl lithium (2.7 mL, 8.1 mmol, 3 M solution in hexanes) was added through a syringe while the temperature was allowed to rise to approximately −10° C. The heterogeneous solution was recooled to −75° C. and acid chloride, Intermediate 169, prepared above in anhydrous THF (5 mL) was added over 10-15 minutes. The resulting mixture was stirred for 3 hours and then poured into a 100 mL separatory funnel containing ethyl acetate (50 mL) and ice-cold 1N HCl (5 mL). The layers were separated and the aqueous layer was extracted with ethyl acetate (50 mL×2). The organic layers were combined and washed with saturated aqueous sodium bicarbonate solution (2×50 mL) and brine (50 mL). The solvent was removed under vacuum to afford a purple solid, which was purified by RP-HPLC (Symmetry C18 column(19×300 mm, 7 μm); using a binary solvent mixture of 10 mM NH₄OAc (A)/CH₃CN (B) (0-20 min 0-40% B, 20-30 min^{.} 40% B and 30-40 min: 40-100% B; flow rate of 15 mL/min; Separation was monitored at 254 nm) to get the title compound as a semi-solid (100 mg).

| | | Mass spectrum and ¹H | |
|---|---|---|---|
| Compound | Structure | NMR | SM |
| Intermediate 170 | 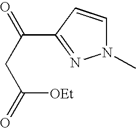 ethyl 3-(1-methyl- 1H-pyrazol-3-yl)-3- oxopropanoate | MS(ES): 197 (M + 1) for C₉H₁₂N₂O₃. 300 MHz, CDCl₃: δ 1.27 (t, J = 7.14 Hz, 3H), 3.97 (s, 3H), 4.02 (s, 2H), 4.21 (q, J = 7.11 Hz, 2H), 6.83 (d, J = 2.34 Hz, 1H), 7.39 (d, J = 2.34 Hz, 1H). | Intermediate 169 1-methyl-1H- pyrazole-3- carbonyl chloride |

### Intermediate 171: ethyl 2-chloro-3-(1-methyl-1H-pyrazol-3-yl)-3-oxopropanoate

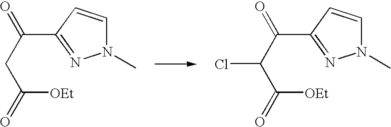

To a solution of ethyl 3-(1-methyl-1H-pyrazol-3-yl)-3-oxopropanoate (Intermediate 170, 100 mg, 0.510 mmol) in ethyl acetate (5 mL) was added N-chlorosuccinimide (75 mg, 0.56 mmol, 1.1 equiv) and Amberlyst-15 resin (100 mg). The resulting mixture was stirred for 4 hours at room temperature. The solid was removed by filtration and the solvent was removed in vacuo to afford the title compound (100 mg) which was used for the next step without further purification.

| | | Mass spectrum and ¹H | |
|---|---|---|---|
| Compound | Structure | NMR | SM |
| Intermediate 171 | 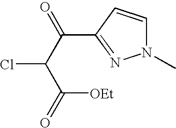 ethyl 2-chloro-3-(1- methyl-1H-pyrazol- 3-yl)-3- oxopropanoate | MS(ES): 231 (M + 1) for C₉H₁₁ClN₂O₃. 300 MHz, CDCl₃: δ 1.28 (t, J = 7.11 Hz, 3H), 4.00 (s, 3H), 4.27 (q, J = 7.26 Hz, 2H), 5.91 (s, 1H), 6.91 (d, J = 2.40 Hz, 1H), 7.43 (d, J = 2.37 Hz, 1H). | Intermediate 170 ethyl 3-(1- methyl-1H- pyrazol-3-yl)-3- oxopropanoate |

### Intermediate 173: (1,3-trans)-tert-butyl 3-(3-bromophenyl)-2,2-dimethylcyclopropanecarboxylate

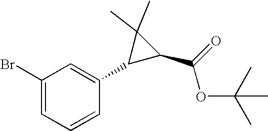

Isopropyltriphenylphosphonium iodide (1.621 g, 3.75 mmol) was suspended in THF (8 ml) and cooled to −78° C. 2.4 M butyllithium in hexanes (1.563 mL, 3.75 mmol) was added dropwise over 2 minutes. The mixture was then stirred over an ice-water bath 30 min to give a clear dark red solution. The mixture was cooled to −78° C. and a solution of (E)-tert-butyl 3-(3-bromophenyl)acrylate (590 mg, 2.08 mmol) in THF (3 ml) was added. The mixture was cooled over an ice-water bath and allowed to slowly warm to room temperature overnight. Methyl iodide (0.6 mL, 9.60 mmol) was added to the mixture followed by stirring at room temperature 1 hour. The mixture was poured into 0.5M HCl, extracted with EtOAc, dried over MgSO₄ and evaporated. The residue was dissolved in dichloromethane and filtered over silica gel, rinsed with 200 ml 5% ethyl acetate in hexane. The filtrate was evaporated to give the crude product as a yellow oil: (550 mg).

¹H NMR (400 MHz, CHLOROFORM-D) δ ppm 0.90 (s, 3H), 1.34 (s, 3H), 1.48 (s, 9H), 1.86 (d, 1H), 2.56 (d, 1H), 7.06-7.17 (m, 2H), 7.29-7.35 (m, 2H).

### Intermediate 174: (1,3-trans)-tert-butyl 2,2-dimethyl-3-(3-(4,4,5,5-tetramethyl-1,3,2-dioxaborolan-2-yl)phenyl)cyclopropanecarboxylate

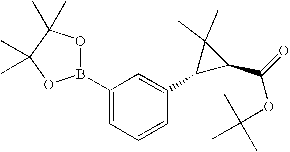

(1,3-trans)-tert-butyl 3-(3-bromophenyl)-2,2-dimethylcyclopropanecarboxylate) Intermediate 173 (1.3 g, 4.00 mmol), 4,4,4′,4′,5,5,5′,5′-octamethyl-2,2′-bi(1,3,2-dioxaborolane (2.030 g, 7.99 mmol), potassium acetate (1.177 g, 11.99 mmol), PdCl2(dppf)-CH₂Cl₂ Adduct (0.326 g, 0.40 mmol) and dioxane (10 mL) were combined and degassed by bubbling an argon stream through the mixture for 10 minutes. The mixture was warmed over a 90° C. heating block 1 h 15 m. The mixture was allowed to cool, diluted to ˜40 ml with CH₂Cl₂, filtered and evaporated then applied to a 40 g silica cartridge and eluted with 0 to 10% ethyl acetate/hexanes. The title compound was obtained as a thick clear oil (1.124 g) 1H NMR (400 MHz, CHLOROFORM-D) δ ppm 0.89 (s, 3H), 1.25 (s, 3H), 1.34 (s, 12H), 1.48 (s, 9H), 1.93 (d, 1H), 2.61 (d, 1H), 7.25 (d, 1H), 7.28 (t, 1H), 7.59 (s, 1H), 7.64 (d, 1H)

The intermediates in the table below were prepared using the procedure described above and the specified starting material.

| | | Mass spectrum and | |
|---|---|---|---|
| Compound | Structure | ¹H NMR | SM |
| Intermediate 175 | 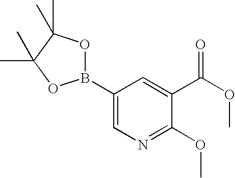 methyl 2-methoxy-5- (4,4,5,5-tetramethyl- 1,3,2-dioxaborolan-2-yl) nicotinate | MS: ES+ 294 for C₁₄H₂₀BNO₅ 1H NMR (300 MHz, DMSO-d₆) δ ppm 1.30 (s, 12 H) 3.81 (s, 3 H) 3.96 (s, 3 H) 8.30 (d, J = 1.88 Hz, 1 H) 8.55 (d, J = 2.07 Hz, 1 H) | Methyl 5-bromo- 2-methoxy nicotinate |

Intermediate 176: (1,3-trans)-tert-butyl 3-(3-(2-(3-chloro-4-fluorophenylamino)-4-(3-(trifluoromethyl)-1H-pyrazol-1-yl)pyrimidin-5-yl)phenyl)-2,2-dimethylcyclopropanecarboxylate

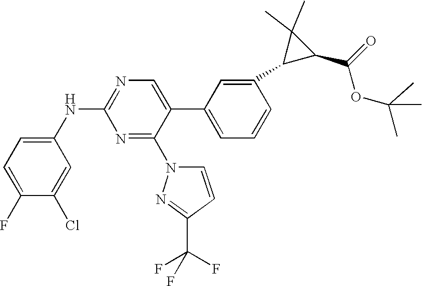

(1S,3S)-tert-butyl 2,2-dimethyl-3-(3-(4,4,5,5-tetramethyl-1,3,2-dioxaborolan-2-yl)phenyl)cyclopropanecarboxylate Intermediate 174 (300 mg, 0.81 mmol), 5-bromo-N-(3-chloro-4-fluorophenyl)-4-(3-(trifluoromethyl)-1H-pyrazol-1-yl)pyrimidin-2-amine [Intermediate 115] (200 mg, 0.46 mmol), Tris(dibenzylideneacetone)dipalladium(0) (41.9 mg, 0.05 mmol), 2-Dicyclohexylphosphino-2′,4′,6′-triisopropylbiphenyl (65.5 mg, 0.14 mmol), Na₂CO₃ (72.8 mg, 0.69 mmol), acetonitrile (3 mL) and water (0.750 mL) were combined and degassed with an argon stream for 10 min. The mixture was warmed at 80° C. 1 h 15 m, allowed to cool, diluted with acetonitrile, filtered and adsorbed on 15 ml silica gel. Flash chromatography (40 g cartridge) 20 to 100% dichloromethane/hexane gave the pure title compound (35 mg). MS: ES+602 for C₃₀H₂₈ClF₄N₅O₂.

1H NMR (400 MHz, CHLOROFORM-D) δ ppm 0.86 (s, 3H), 1.32 (s, 3H), 1.46 (s, 9H), 1.79 (d, 1H), 2.57 (d, 1H), 6.56 (s, 1H), 6.92 (s, 1H), 7.00 (d, 1H), 7.09-7.21 (m, 2H), 7.25-7.32 (m, 2H), 7.35-7.41 (m, 1H), 7.85-7.95 (m, 2H), 8.52 (s, 1H).

### Intermediate 177: (E)-tert-butyl 3-(5-bromopyridin-3-yl)acrylate

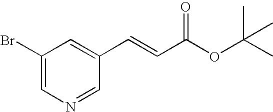

tert-Butyl 2-(diethoxyphosphoryl)acetate (2.291 mL, 9.75 mmol) and tetrahydrofuran (5 mL) were combined and cooled over a dry-ice ethanol bath at −70 C then Sodium bis(trimethylsilyl)amide, 2M solution in THF (4.82 mL, 9.63 mmol) was added dropwise over 5 minutes to give a clear yellow solution. The mixture was stirred over the cold bath 30 minutes, then a solution of 5-bromonicotinaldehyde (1.629 g, 8.76 mmol) in 5 ml THF was added. The cold bath was removed and the mixture was stirred 20 minutes. The mixture was poured into 0.5M HCl and extracted ethyl acetate, washed with saturated sodium chloride, dried over MgSO4 and evaporated to give the title compound as a light yellow solid (2.78 g). 1H NMR (400 MHz, CHLOROFORM-D) δ ppm 1.52 (s, 9H), 6.44 (d, 1H), 7.48 (d, 1H), 7.98 (d, 1H), 8.55-8.73 (m, 2H)

### Intermediate 178: (1,2-trans)-tert-butyl 2-(5-bromopyridin-3-yl)cyclourouanecarboxylate

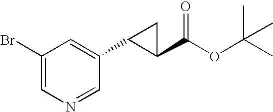

Trimethylsulfoxonium iodide (1.084 g, 4.93 mmol) was combined with dimethylsulfoxide (5 ml) and 60% sodium hydride dispersion in oil (0.183 g, 4.58 mmol) and stirred at room temperature for 50 minutes to give a clear solution, then (E)-tert-butyl 3-(5-bromopyridin-3-yl)acrylate Intermediate 177 (1 g, 3.52 mmol) was added together with dimethylsulfoxide (1 ml) to give a yellow-orange suspension which was stirred at room temperature for 45 minutes. The mixture was poured into 50 ml water, extracted twice with ethyl acetate and the organic layer was dried over magnesium sulfate and evaporated to give a clear oil. Purification by flash chromatography, 0 to 15% ethyl acetate in hexane gave the title compound as a white solid (455 mg). 1H NMR (400 MHz, CHLOROFORM-D) δ ppm 1.20-1.28 (m, 1H), 1.47 (s, 9H), 1.56-1.63 (m, 1H), 1.82-1.90 (m, 1H), 2.38-2.45 (m, 1H),7.51 (s, 1H), 8.35 (d, 1H), 8.50 (d, 1H).

### Intermediate 179: (1,2-trans)-tert-butyl 2-(5-(4,4,5,5-tetramethyl-1,3,2-dioxaborolan-2-yl)pyridin-3-yl)cyclopropanecarboxylate

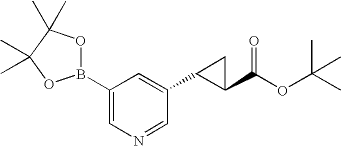

(1,2-trans)-tert-butyl 2-(5-bromopyridin-3-yl)cyclopropanecarboxylate Intermediate 178 (0.435 g, 1.46 mmol), 4,4,4′,4′,5,5,5′,5′-octamethyl-2,2′-bi(1,3,2-dioxaborolane) (0.741 g, 2.92 mmol), potassium acetate (0.430 g, 4.38 mmol), PdCl2(dppf)-dichloromethane adduct (0.119 g, 0.15 mmol) and dioxane (5 mL) were combined and degassed by bubbling and argon stream through the mixture for 10 minutes. The mixture was warmed at 90° for 1.5 hours, allowed to cool and filtered. The solids were rinsed with dichloromethane and the filtrate was evaporated. The residue was filtered over 25 ml silica gel, eluting with dichloromethane (200 ml), then 25% ethyl acetate in dichloromethane (200 ml). The filtrate was discarded before the silica gel was further eluted with 200 ml 5% methanol in dichloromethane. Evaporation gave the title compound as a crude brown solid (270 mg). ¹H NMR (400 MHz, CHLOROFORM-D) δ ppm 0.79-0.99 (m, 1H), 1.34 (s, 12H), 1.46 (s, 9H), 1.51-1.59 (m, 1H), 1.83-1.90 (m, 1H), 2.38-2.47 (m, 1H), 7.68 (s, 1H), 8.49 (m, 1H), 8.76 (m, 1H).

### Intermediate 180: (1,2-trans)-tert-butyl 2-(5-(2-(3-chloro-4-fluorophenylamino)-4-(3-(trifluoromethyl)-1H-pyrazol-1-yl)pyrimidin-5-yl)pyridin-3-yl)cyclopropanecarboxylate

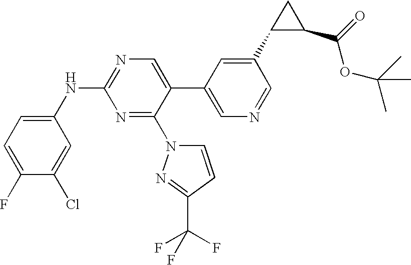

(1R,2R)-tert-butyl 2-(5-(4,4,5,5-tetramethyl-1,3,2-dioxaborolan-2-yl)pyridin-3-yl)cyclopropanecarboxylate, Intermediate 179 (270 mg, 0.55 mmol), 5-bromo-N-(3-chloro-4-fluorophenyl)-4-(3-(trifluoromethyl)-1H-pyrazol-1-yl)pyrimidin-2-amine [Intermediate 115] (159 mg, 0.36 mmol), Tris(dibenzylideneacetone) dipalladium(0) (33.4 mg, 0.04 mmol), 2-Dicyclohexylphosphino-2′,4′,6′-triisopropylbiphenyl (52.2 mg, 0.11 mmol), sodium carbonate (58.0 mg, 0.55 mmol), acetonitrile (2 mL) and water (0.500 mL) were combined and degassed with an argon stream for 10 min. The mixture was warmed at 80° C. for 1 hour 15 minutes, allowed to cool, diluted with acetonitrile, filtered and adsorbed on silica gel. Purification by flash chromatography (25 g cartridge) 0.5 to 5% methanol in dichloromethane gave the solid title compound (110 mg). MS: ES+ 575 for C₂₇H₂₃ClF₄N₆O₂.

1H NMR (400 MHz, DMSO-D6) δ ppm 1.21-1.30 (m, 2H), 1.41 (s, 9H), 1.78-1.86 m, 1H), 2.33-2.42 (m, 1H), 7.02 (d, 1H), 7.23 (s,1H), 7.41 (t, 1H), 7.65-7.75 (m, 1H), 8.08 (dd, 1H), 8.21 (d, 1H), 8.45 (d, 2H), 8.81 (s, 1H), 10.42 (s, 1H).

### Intermediate 181: (1,2-trans)-tert-butyl 2-(3-bromophenyl)cyclopropanecarboxylate

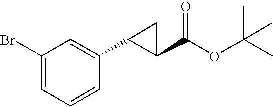

The title compound was prepared using the general method described above for Intermediate 178 using (E)-tert-butyl 3-(3-bromophenyl)acrylate as a starting material. ¹H NMR (300 MHz, CHLOROFORM-D) δ ppm 0.76-0.94 (m, 1H), 1.16-1.23 (m, 1H), 1.46 (s, 9H), 1.77-1.86 (m, 1H), 2.33-2.43 (m, 1H), 7.01 (d, 4H), 7.12 (t, 1H), 7.21 (s, 1H), 7.31 (d, 1H).

### Intermediate 182: (1,2-trans)-tert-butyl 2-(3-(4,4,5,5-tetramethyl-1,3,2-dioxaborolan-2-yl)phenyl)cyclopropanecarboxylate

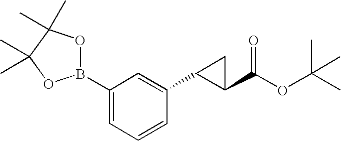

The title compound was prepared using the general method described above for Intermediate 178 using Intermediate 181 as a starting material. ¹H NMR (400 MHz, CHLOROFORM-D) δ ppm 0.80-0.93 (m, 1H), 1.45 (s, 12H), 1.45 (s, 9H), 1.47-1.53 (m, 1H), 1.79-1.89 (m, 1H), 2.40-2.50 (m, 1H), 7.17 (d, 1H), 7.28 (d, 1H), 7.52 (s, 1H), 7.63 (d, 1H).

### Intermediate 183: (1,2-trans)-tert-butyl 2-(3-(2-(3-chloro-4-fluorophenylamino)-4-(3-(trifluoromethyl)-1H-pyrazol-1-yl)pyrimidin-5-yl)phenyl)cyclopropanecarboxylate

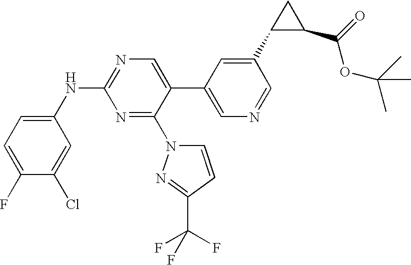

The title compound was prepared using the general method described above for Intermediate 178 using Intermediate 182 and Intermediate 115 as starting materials. MS: ES+ 574 for C₂₇H₂₃ClF₄N₆O₂.

¹H NMR (400 MHz, DMSO-D6) δ ppm 1.17-1.22 (m, 1H), 1.30-1.37 (m, 1H), 1.40 (s, 9H), 1.68-1.76 (m, 1H), 2.23-2.33 (m, 1H), 6.79 (s,1H), 6.96 (d, 1H), 7.00 (d, 1H), 7.13-7.18 (m, 1H), 7.24 (t, 1H), 7.39 (t, 1H), 7.65-7.73 (m, 1H), 8.12 (dd, 1H), 8.25 (s, 1H), 8.79 (s, 1H), 10.38 (s, 1H).

### Intermediate 184: methyl 5-bromo-2-(methylamino)nicotinate

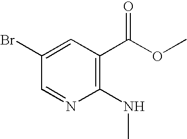

Methyl 5-bromo-2-chloronicotinate (1 g, 3.99 mmol) and methylamine, 2M solution in THF (4 ml, 8.00 mmol) were combined and warmed in a microwave reactor at 125° C. for 1 hour. The mixture was combined with 50 ml ethyl acetate, washed with 0.2M HCl, dried over magnesium sulfate and evaporated to give the title compound as an off-white solid (0.956 g).

¹H NMR (300 MHz, DMSO-d6) δ ppm 2.93 (d, 3H), 3.82 (s, 3H), 7.87 (br. s., 1H), 8.12 (d, 1H), 8.39 (d, 1H)

### Intermediate 185: methyl 5-bromo-2-(dimethylamino)nicotinate

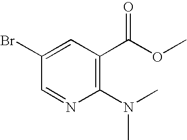

The title compound was prepared using the general method described above for Intermediate 184 using dimethylamine, 2M solution in THF as a reagent. ¹H NMR (300 MHz, DMSO-d6) d ppm 2.92 (s, 6H), 3.82 (s, 3H), 7.97 (d, 1H), 8.30 (d, 1H)

### Intermediate 186: methyl 5-bromo-2-(1H-1,2,4-triazol-1-yl)nicotinate

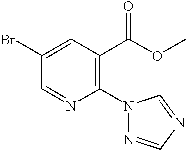

4H-1,2,4-triazole (0.4 g, 5.79 mmol) was dissolved in NMP (5 mL) then sodium hydride (60% dispesion in oil) (200 mg, 5.00 mmol) was added. The mixture was stirred at room temperature for 30 minutes, then methyl 5-bromo-2-chloronicotinate (1 g, 3.99 mmol) was added. The mixture was stirred for 2 hours, diluted with water, extracted with ethyl acetate, washed with saturated sodium chloride, dried over magnesium sulfate and evaporated. Purification of the residue by flash chromatography (25 g cartridge, 20 to 60% ethyl acetae/hexanes) gave the title compound as an off-white solid (0.42 g).

MS: ES+ 284 for C₉H₇BrN₄O₂.

¹H NMR (400 MHz, DMSO-d6) δ ppm 3.76 (s, 3H), 8.29 (s, 1H), 8.56 (d, 1H), 8.89 (d, 1H), 9.29 (s, 1H).

### Intermediate 187 methyl 5-bromo-2-(1H-pyrazol-1-yl)nicotinate

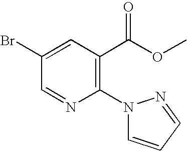

The title compound was prepared using the general method described above for Intermediate 186 using 1H-pyrazole as a reagent.

MS: ES+ 283 for C₁₀H₈BrN₃O₂.

¹H NMR (400 MHz, DMSO-d6) δ ppm 3.75 (s, 3H), 6.53-6.64 (m, 1H), 7.81 (d, 1H), 8.41 (d, 1H), 8.51 (d, 1H), 8.76 (d, 1H).

### Intermediate 188 methyl 5-bromo-2-(2-(methylsulfonyl)ethylamino)nicotinate

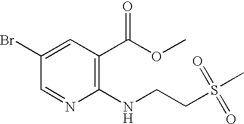

Methyl 5-bromo-2-chloronicotinate (800 mg, 3.19 mmol), 2-(methylsulfonyl)ethanamine hydrochloride (586 mg, 3.67 mmol), NMP (4 mL) and N,N-diisopropylethylamine (0.893 mL, 5.11 mmol) were combined and warmed over at 80° for 18 h, diluted with water (50 ml) to give a solid precipitate which was collected and rinsed with water, then 1:1 ether/hexanes to give the title compound as a beige solid (0.687 g).

MS: ES+ 338 for C₁₀H₁₃BrN₂O₄S.

¹H NMR (400 MHz, DMSO-d6) δ ppm 3.02 (s, 3H), 3.40 (t, 2H), 3.83 (s, 3H), 3.90 (q, 2H), 8.18 (d, 1H), 8.16 (t, 1H), 8.42 (d, 1H)

### Intermediate 189 methyl 5-bromo-2-(1H-imidazol-1-yl)nicotinate

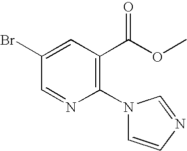

Methyl 5-bromo-2-chloronicotinate (1 g, 4 mmol) and imidazole (820 mg, 12 mmol) combined with NMP (5 mL) and warmed at 70° C. for 17 hours, then at 100° C. for 5 hours. The mixture was diluted with water, extracted with ethyl acetate, washed with saturated sodium chloride, dried over magnesium sulfate and evaporated. The residue was purified by flash chromatography (25 g cartridge, 10 to 40% acetonitrile in dichloromethane) to give the title compounds as an off-white solid: 236 mg.

MS: ES+ 283 for C₁₀H₈BrN₃O₂.

¹H NMR (400 MHz, DMSO-d6) δ ppm 3.78 (s, 3H), 7.07 (s, 1H), 7.50 (s, 1H), 8.03 (s, 1H), 8.58 (d, 1H), 8.90 (d, 1H)

### Intermediate 190: methyl 5-bromo-1-methyl-6-oxo-1,6-dihydropyridine-3-carboxylate

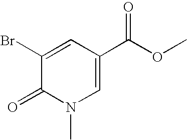

Methyl 5-bromo-6-oxo-1,6-dihydropyridine-3-carboxylate (2 g, 8.62 mmol) was combined with DMF (20 mL) and triethylamine (3.60 mL, 25.86 mmol) to give a clear solution which was cooled over an ice-water bath. Dimethylsulfate (2.452 mL, 25.86 mmol) was added to the cold solution dropwise over several minutes. The clear solution was removed from the cold bath and stirred at room temperature for 1.5 hours to give a suspension. The mixture was diluted with 0.2M NaOH to 200 ml and the solids were filtered and rinsed with water to give the title compound as a white solid (0.84 g).

¹H NMR (400 MHz, CHLOROFORM-d) δ ppm 3.68 (s, 3H), 3.88 (s, 3H), 8.19 (d, 1H), 8.27 (d, 1H).

### General Procedure for Reaction of Amino Acids with 1,3-dibromobenzene

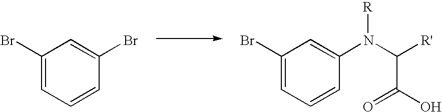

A suspension of 1,3-dibromobenzene (1 eq), amino acid (2-2.5 eq), CuI (20 mol %) and potassium carbonate (3 eq) in DMF (5 mL) was taken in sealed tube, degassed and heated to 90° C. overnight under inert atmosphere. The reaction mixture was then cooled to RT and filtered through a celite bed. The filtrate was acidified with 1.5 N HCl. The solvent was removed under vacuum and the crude mixture taken in CHCl₃ (50 mL), washed with water and brine, dried over Na₂SO₄, filtered and concentrated. The crude material was purified by silica gel column chromatography using chloroform: methanol (9:1) as an eluent to give the product.

### The Compounds in the Table Below were Prepared Using this General Procedure and the Starting Material Specified

| Compound | Structure | Mass spectrum and ¹H NMR | SM |
|---|---|---|---|
| Intermediate 191 | 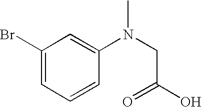 N-(3-bromophenyl)-N- methylglycine | MS(ES): 244 (M) and 246 (M + 2) for C₉H₁₀BrNO₂. 400 MHz, DMSO-d₆: δ 2.94 (s, 3H), 4.08 (s, 2H), 6.65 (t, J = 7.20 Hz, 1H), 6.78 (t, J = 3.60 Hz, 1H), 7.09 (t, J = 8.40 Hz, 1H), 7.95-8.07 (m, 1H), 12.71 (s, 1H). | N-methyl glycine |
| Intermediate 192 | 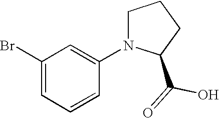 1-(3-bromophenyl)-L- proline | Taken to the next step based on LCMS without further purification. MS(ES): 270 (M) and 272 (M + 2) for C₁₁H₁₂BrNO₂. | L-proline |
| Intermediate 193 | 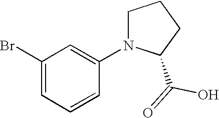 1-(3-bromophenyl)-D- proline | Taken to the next step based on LCMS withour further purification. MS(ES): 270 (M) and 272 (M + 2) for C₁₁H₁₂BrNO₂. (74% pure by LCMS) | D-proline |
| Intermediate 194 | 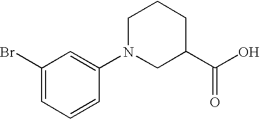 1-(3- bromophenyl(piperidine- 3-carboxylic acid | MS(ES): 284 (M) and 286 (M + 2) for C₁₂H₁₄BrNO₂. 400 MHz, DMSO-d₆: δ 3.15 (d, J = 14.12 Hz, 1H), 3.28- 3.29 (m, 2H), 3.55-3.71 (m, 5H), 7.37 (t, J = 12.16 Hz, 1H), 7.54-7.60 (m, 2H), 7.95 (q, J = 3.44 Hz, 1H), 8.74 (s, 1H), 10.29 (s, 1H). | piperidine-3- carboxylic acid |
| Intermediate 195 | 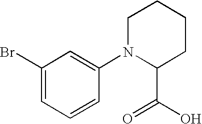 1-(3- bromophenyl)piperidine- 2-carboxylic acid | Taken to the next step based on LCMS without further purification. MS(ES): 284 (M) and 286 (M + 2) for C₁₂H₁₄BrNO₂. (65% pure by LCMS) | piperidine-2- carboxylic acid |

### General Procedure for Esterification of N-aryl Amino Acids

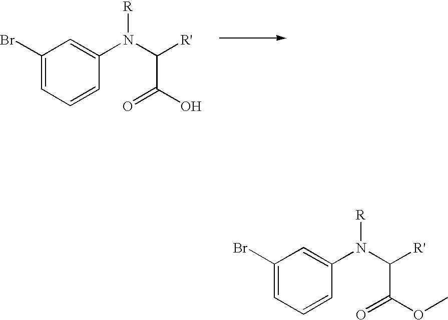

To a suspension of amino acid derivative (1.5 eq) taken in excess of MeOH at 0° C., was added thionyl chloride (1 vol) slowly and the reaction mixture refluxed for 2 h. The solvent was removed under vacuum and the crude mixture was taken in EtOAc (30 mL), washed with aq. NaHCO₃ solution, water and brine, dried over Na₂SO₄, filtered and concentrated. The crude material was taken to the next step as such without further purification.

### The compounds in the Table Below were Prepared Using this General Procedure and the Starting Material Specified

| Compound | Structure | Mass spectrum and ¹H NMR | SM |
|---|---|---|---|
| Intermediate 196 | 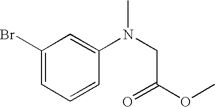 methyl N-(3- bromophenyl)-N- methylglycinate | Taken to the next step based on LCMS without further purification. MS(ES): 258 (M) and 260 (M + 2) for C₁₀H₁₂BrNO₂. | N-(3-bromo- phenyl)-N- methylglycine Intermediate 191 |
| Intermediate 197 | 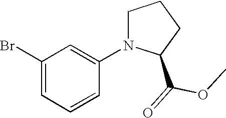 methyl 1-(3- bromophenyl)-L- prolinate | MS(ES): 284 (M) and 286 (M + 2) for C₁₂H₁₄BrNO₂. 300 MHz, CDCl₃: δ 2.06-2.18 (m, 4H), 3.33-3.36 (m, 1H), 3.50-3.62 (m, 1H), 3.73 (s, 3H), 4.24 (dd, J = 2.37, 8.28 Hz, 1H), 6.43 (dd, J = 1.89, 8.32 Hz, 1H), 6.68 (t, J = 2.04 Hz, 1H), 6.81-6.84 (m, 1H), 7.06 (t, J = 8.13 Hz, 1H). | 1-(3- bromophenyl)- L-proline Intermediate 192 |
| Intermediate 198 | 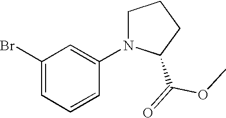 methyl 1-(3- bromophenyl)-D- prolinate | Taken to the next step based on LCMS without furhter purification. MS(ES): 284 (M) and 286 (M + 2) for C₁₂H₁₄BrNO₂. (75% pure by LCMS) | 1-(3- bromophenyl)- D-proline Intermediate 193 |
| Intermediate 199 | 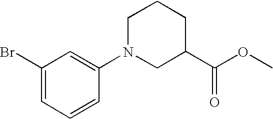 methyl 1-(3- bromophenyl) piperidine-3- carboxylate | MS(ES): 298 (M) and 300 (M + 2) for C₁₃H₁₆BrNO₂. 300 MHz, DMSO-d₆: δ 1.42-1.73 (m, 3H), 1.85-1.90 (m, 1H), 2.57- 2.60 (m, 1H), 2.75-2.92 (m, 1H), 2.96-3.03 (m, 1H), 3.40-3.58 (m, 1H), 3.61 (s, 3H), 3.37-3.42 (m, 1H), 6.89 (t, J = 7.74 Hz, 2H), 6.94 (d, J = 8.57 Hz, 1H), 7.03-7.12 (m, 1H). | 1-(3-bromo- phenyl)piper- idine-3- carboxylic acid Intermediate 194 |
| Intermediate 200 | 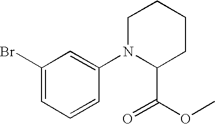 methyl 1-(3- bromophenyl) piperidine-2- carboxylate | Taken to the next step based on LCMS without further purification. MS(ES): 298 (M) and 300 (M + 2) for C₁₃H₁₆BrNO₂. | 1-(3- bromophenyl) piperidine- 2-carboxylic acid Intermediate 195 |

### General Procedures for the Preparation of Boronate Esters from Aryl Bromides

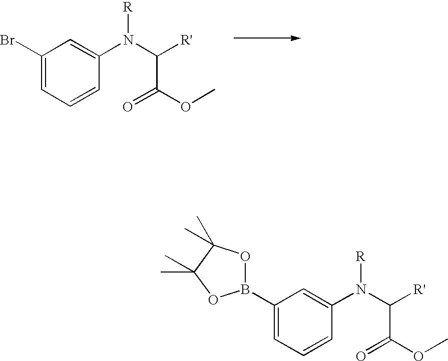

### Method I:

A suspension of aryl bromide (1 eq), bis(pinacolato)diboron (2 eq), [1,1′-bis(diphenylphosphino)ferrocene]dichloropalladium(II) complex with CH₂Cl₂ (20 mol %) and potassium acetate (3 eq) was taken in dioxane and it was degassed for 10 min. Then the reaction mixture was heated overnight at 90° C. The reaction mixture was cooled to room temperature, filtered through a celite bed, washed with ethyl acetate twice and concentrated in vacuo. The residue was diluted with ethyl acetate (2×), washed with water (1×) and brine (1×), dried over Na₂SO₄ and concentrated in vacuo. The crude mass was purified by 60-120 silica gel column chromatography using ethyl acetate/hexanes and as eluent to give the product.

### Method II:

A suspension of aryl bromide (1 eq), bis(pinacolato)diboron (3 eq), palladium(II) acetate (20-40 mol %), 2-dicyclohexylphosphino-2′,4′,6′-triisopropylbiphenyl (20 mol %) and triethylamine (3 eq) was taken in dioxane and it was degassed for 10 min. Then the reaction mixture was heated overnight at 90-100° C. The reaction mixture was cooled to room temperature, diluted with water and extracted with ethyl acetate (2×). The organic layer was washed with water (1×) and brine (1×), dried over Na₂SO₄ and concentrated in vacuo. Then the crude mass was purified by 60-120 silica gel column chromatography using ethyl acetate/hexanes and as eluent to give the product.

### The Compounds in the Below Table were Prepared Using this General Procedure and the Starting Material Specified

| Compound | Structure | Mass spectrum and ¹H NMR | SM |
|---|---|---|---|
| Intermediate 201^{b)} | 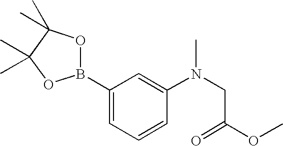 methyl N-methyl-N-[3- (4,4,5,5-tetramethyl- 1,3,2-dioxaborolan-2- yl)phenyl]glycinate | The compound was taken to the next step on the basis of LCMS MS(ES): 306 (M + 1) for C₁₆H₂₄BNO₄. (35% purity by LCMS). | methyl N-(3- bromophenyl)- N- methylglycinate Intermediate 196 |
| Intermediate 202^{a)} | 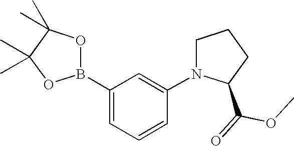 methyl 1-[3-(4,4,5,5- tetramethyl-1,3,2- dioxaborolan-2- yl)phenyl]-L-prolinate | MS(ES): 332 (M + 1) for C₁₈H₂₆BNO₄. 300 MHz, CDCl₃: δ 1.34 (s, 12H), 2.13-2.19 (m, 4H), 3.41- 3.44 (m, 1H), 3.50-3.64 (m, 1H), 3.71 (s, 3H), 4.29-4.30 (m, 1H), 6.64 (d, J = 6.30 Hz, 1H), 7.04 (d, J = 5.64 Hz, 1H), 7.16-7.27 (m, 2H). | methyl 1-(3- bromophenyl)- L-prolinate Intermediate 197 |
| Intermediate 203^{a)} | 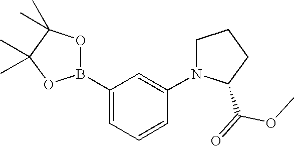 methyl 1-[3-(4,4,5,5- tetramethyl-1,3,2- dioxaborolan-2- yl)phenyl]-D-prolinate | MS(ES): 332 (M + 1) for C₁₈H₂₆BNO₄. 300 MHz, DMSO-d₆: δ 1.15 (s, 12H), 2.26-2.31 (m, 2H), 2.35-2.42 (m, 2H), 3.62 (s, 3H), 4.00-4.20 (m, 2H), 4.31 (d, J = 7.95 Hz, 1H), 6.78 (d, J = 7.53 Hz, 1H), 6.95 (d, J = 7.32 Hz, 1H), 7.16-7.21 (m, 2H). | methyl 1-(3- bromophenyl)- D-prolinate Intermediate 198 |
| Intermediate 204^{b)} | 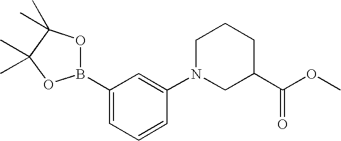 methyl 1-[3-(4,4,5,5- tetramethyl-1,3,2- dioxaborolan-2- yl)phenyl]piperidine-3- carboxylate | Taken to the next step based on LCMS without further purification. MS(ES): 346 (M + 1) for C₁₉H₂₈BNO₄. (40% pure by LCMS) | methyl 1-(3- bromophenyl) piperidine-3- carboxylate Intermediate 199 |
| Intermediate 205^{b)} | 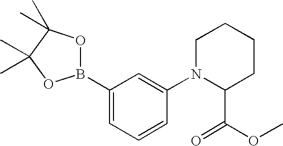 methyl 1-[3-(4,4,5,5- tetramethyl-1,3,2- dioxaborolan-2- yl)phenyl]piperidine-2- carboxylate | The compound was taken to the next step on the basis of LCMS. MS(ES): 346 (M + 1) for C₁₉H₂₈BNO₄. (80% pure by LCMS) | methyl 1-(3- bromophenyl) piperidine-2- carboxylate Intermediate 200 |

^{a)}Method I;
^{b)}Method II

### Intermediate 206: N-(3-bromophenyl)glycine

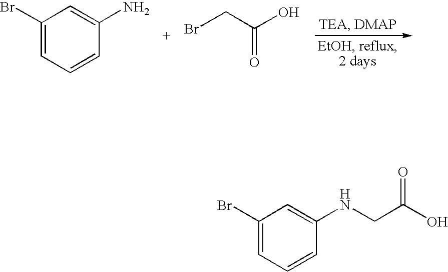

To a suspension of 3-bromoaniline (5.8 mmol, 1 g), and bromoacetic acid (8.7 mmol, 1.2 g) in ethanol (50 mL), was added triethylamine (17.3 mmol, 1.75 g) and 4-(Dimethylamino)pyridine (1.7 mmol, 0.2 g). The mixture was refluxed for 2 days. The reaction mixture was concentrated in vacuo, acidified with 1.5 N HCl and extracted with ethyl acetate (50 mL).The organic layer was washed with brine solution (25 mL), dried over Na₂SO₄ and concentrated in vacuo. The crude mass was purified by 60-120 silica gel column chromatography using chloroform and methanol (2%) as eluent to give the title compound (0.5 g). Taken for next step on basis of LCMS.

| | | Mass spectrum and ¹H | |
|---|---|---|---|
| Compound | Structure | NMR | SM |
| Intermediate 206 | 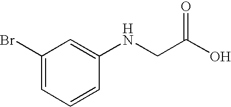 N-(3-bromophenyl) glycine | Taken to the next step based on LCMS without further purification. MS(ES): 230 (M) and 232 (M + 2) for C₈H₈BrNO₂ (57% pure by LCMS). | 3-bromoaniline and bromoacetic acid |

### Intermediate 207: methyl 1-(3-bromophenyl)pyrrolidine-3-carboxylate

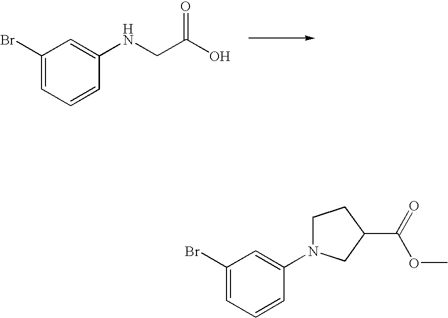

A suspension of N-(3-bromophenyl)glycine Intermediate 206 (3.04 mmol, 0.7 g), 30% aq. formaldehyde solution (4.56 mmol, 0.14 g) and methyl acrylate (4.65 mmol, 0.4 g) in toluene (5 mL) was refluxed for 2 days. The reaction mixture was concentrated in vacuo and diluted with ethyl acetate (10 mL). The ethyl acetate layer was washed with water (5 mL), brine solution (5 mL), dried over Na₂SO₄ and concentrated in vacuo. The crude mass was purified by 60-120 silica gel column chromatography using 8% ethyl acetate/hexanes as eluent to give the title compound (0.15 g). Taken for next step on basis of LCMS.

| | | Mass spectrum and ¹H | |
|---|---|---|---|
| Compound | Structure | NMR | SM |
| Intermediate 207 | 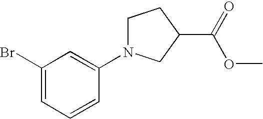 methyl 1-(3- bromophenyl) pyrrolidine-3- carboxylate | Taken to the next step based on LCMS without further purification. MS(ES): 284 (M) and 286 (M + 2) for C₁₂H₁₄BrNO₂ (65% pure by LCMS). | N-(3- bromophenyl) glycine Intermediate 206 |

### Intermediate 208: methyl 1-[3-(4,4,5,5-tetramethyl-1,3,2-dioxaborolan-2-yl)phenyl]pyrrolidine-3-carboxylate and {3-[3-(methoxycarbonyl)pyrrolidin-1-yl]phenyl}boronic acid

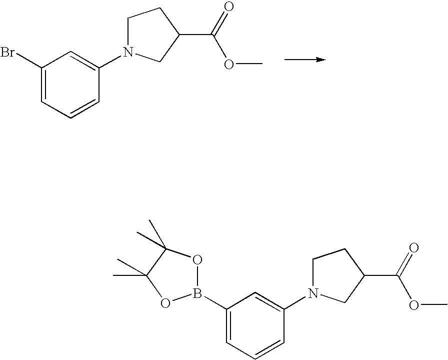

A suspension of methyl 1-(3-bromophenyl)pyrrolidine-3-carboxylate (Intermediate 207, 0.42 mmol, 120 mg), bis(pinacolato)diboron (0.84 mmol, 215 mg), [1,1′-bis(diphenylphosphino)ferrocene]dichloropalladium(II) (0.084 mmol, 61 mg) and potassium acetate (1.26 mmol, 125 mg) was taken in dioxane (10 mL) and it was degassed for 10 min Then the reaction mixture was heated overnight at 90° C. The reaction mixture was cooled to room temperature, filtered through celite bed, washed with ethyl acetate twice and concentrated in vacuo. Then the residue was diluted with ethyl acetate (10 mL), washed with water (5 mL), brine solution (5 mL), dried over Na₂SO₄ and concentrated in vacuo. The crude mass was purified by 60-120 silica gel column chromatography using 10% ethyl acetate/hexanes and as eluent to give 0.1 g of a 72:19 mixture of methyl 1-[3-(4,4,5,5-tetramethyl-1,3,2-dioxaborolan-2-yl)phenyl]pyrrolidine-3-carboxylate and {3-[3-(methoxycarbonyl)pyrrolidin-1-yl]phenyl}boronic acid which was taken for next step on basis of LCMS.

| Compound | Structure | Mass spectrum and ¹H NMR | SM |
|---|---|---|---|
| Intermediate 208 | 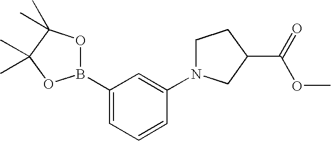 + 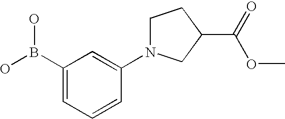 methyl 1-[3-(4,4,5,5- tetramethyl-1,3,2- dioxaborolan-2- yl)phenyl]pyrrolidine- 3-carboxylate and {3-[3- (methoxycarbonyl) pyrrolidin-1- yl]phenyl}boronic acid | Taken to the next step based on LCMS without further purification. MS(ES): 332 (M + 1) for C₁₈H₂₆BNO₄ (72%) and 250 (M + 1) for C₁₂H₁₆BNO₄ (19%) | Intermediate 207 methyl 1-(3- bromophenyl) pyrrolidine-3- carboxylate |

### Intermediate 209: 5-bromo-N-(3,5-dimethoxyphenyl)-4-methoxypyrimidin-2-amine

To a stirred solution of 5-bromo-2-chloro-4-methoxypyrimidine (53.81 mmol, 12 g,) and dimethoxyaniline (54.9 mmol, 8.4 g) in n-BuOH (150 mL) was added dioxane-HCl (12 mL) slowly. The reaction mixture was heated to 110° C. for 3 h. It was then cooled to room temperature, diethyl ether (150 mL) was added and the resulting solid was filtered to yield the title compound (15 g).

| Compound | Structure | Mass spectrum and ¹H NMR | SM |
|---|---|---|---|
| Intermediate 209 | 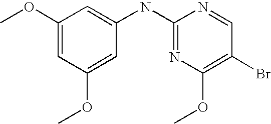 5-bromo-N-(3,5- dimethoxyphenyl)- 4- methoxypyrimidin- 2-amine | MS (ES): 340 (M), 342 (M + 2) for C₁₃H₁₄BrN₃O₃ ¹H NMR (300 MHz, DMSO-d₆): δ 3.70 (s, 6H), 4.00 (s, 3H), 6.12 (s, 1H), 6.14 (s, 1H), 7.01 (s, 1H), 7.02 (s, 1H), 8.36 (s, 1H). | 5-bromo-2-chloro- 4- methoxypyrimidine and 3,5- dimethoxyaniline |

### Intermediate 210: 5-bromo-N-(3-fluorophenyl)-4-methoxypyrimidin-2-amine

To a stirred solution of 5-bromo-2-chloro-4-methoxypyrimidine (11.21 mmol, 2.5 g) and 3-fluoroaniline (12.28 mmol, 1.37 g, 1.18 mL) in 2,2,2-trifluoroethanol (15 mL), trifluoroacetic acid (22.34 mmol, 2.55 g, 1.66 mL) was added and the solution was refluxed at 75° C. with constant stirring. After completion of the reaction, as monitored by TLC, water was added to the reaction mixture and extracted with EtOAc (2×50 mL). The organic layer was washed with water, 10% NaHCO₃ solution and brine, dried over sodium sulfate and concentrated in vacuo to yield the title compound (2.8 g).

| Compound | Structure | Mass spectrum and ¹H NMR | SM |
|---|---|---|---|
| Intermediate 210 | 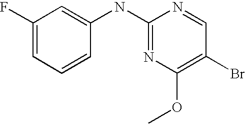 5-bromo-N-(3- fluorophenyl)-4- methoxypyrimidin- 2-amine | MS(ES): 298 (M) and 300 (M + 2) for C₁₁H₉BrFN₃O. 400 MHz, DMSO-d₆: δ 4.01 (s, 3H), 6.77 (td, J = 2.48, 8.44 Hz, 1H), 7.30 (dd, J = 8.12, 15.34 Hz, 1H), 7.47 (d, J = 9.20 Hz, 1H), 7.73 (td, J = 2.16, 12.39 Hz, 1H), 8.41 (s, 1H), 9.96 (s, 1H). | 5-bromo-2-chloro- 4- methoxypyrimidine and 3-fluoroaniline |

Intermediate 211: 5-bromo-2-[(3,5-dimethoxyphenyl)amino]pyrimidin-4-ol

A solution of 5-bromo-N-(3,5-dimethoxyphenyl)-4-methoxypyrimidin-2-amine (Intermediate 209, 14.7 mmol, 5 g) in n-BuOH (50 mL) and dioxane-HCl (15 mL) were heated to 110° C. in a sealed tube and stirred for 9 h. The reaction mixture was cooled to room temperature and BuOH was concentrated under vacuum. To the residue obtained, was added water (50 mL) and neutralized using 10% NaHCO₃ solution. The resulting solid was filtered to yield 3.5 g of 5-bromo-2-[(3,5-dimethoxyphenyl)amino]pyrimidin-4-ol as a white solid.

| Compound | Structure | Mass spectrum and ¹H NMR | SM |
|---|---|---|---|
| Intermediate 211 | 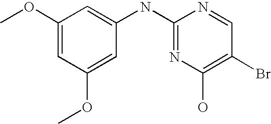 5-bromo-2- [(3,5- dimethoxyphenyl) amino]pyrimidin- 4-ol | MS (ES): 326 (M), 328 (M + 2) for C₁₂H₁₂BrN₃O₃. ¹H NMR (300 MHz, DMSO-d₆): δ 3.74 (s, 6H), 6.21 (s, 1H), 6.75 (s, 2H), 8.05 (s, 1H), 8.88 (s, 1H), 11.35 (s, 1H). | Intermediate 209 5-bromo-N-(3,5- dimethoxyphenyl)- 4- methoxypyrimidin- 2-amine |

### Intermediate 212: 5-bromo-2-[(3-fluorophenyl)amino]pyrimidin-4-ol

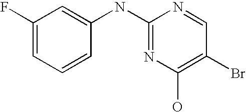

A solution of 5-bromo-N-(3-fluorophenyl)-4-methoxypyrimidin-2-amine (Intermediate 210, 5.03 mmol, 1.5 g) in n-BuOH (15 mL) and dioxane-HCl (4.5 mL) was heated to 110° C. in a sealed tube and stirred for 36 h. The reaction mixture was cooled to room temperature and BuOH was concentrated under vacuum. To the residue obtained, was added water (50 mL) and neutralized using 10% NaHCO₃ solution. The resulting solid was filtered to yield 0.6 g of 5-bromo-2-[(3-fluorophenyl]amino]pyrimidin-4-ol.

| Compound | Structure | Mass spectrum and ¹H NMR | SM |
|---|---|---|---|
| Intermediate 212 | 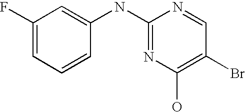 5-bromo-2-[(3- fluorophenyl) amino]pyrimidin-4- ol | MS(ES): 284 (M) and 286 (M + 2) for C₁₀H₇BrFN₃O. 400 MHz, DMSO-d₆: δ 6.79 (td, J = 1.60, 8.40 Hz, 1H), 7.29-7.35 (m, 1H), 7.37 (d, J = 8.40 Hz, 1H), 7.79 (dt, J = 2.00, 12.40 Hz, 1H), 8.08 (s, 1H), 10.44 (br s, 1H), 13.20 (br s, 1H). | Intermediate 210 5-bromo-N-(3- fluorophenyl)-4- methoxypyrimidin- 2-amine |

### General Procedure for the Synthesis of 5-bromo-4-chloro-N-(aryl)pyrimidin-2-amine

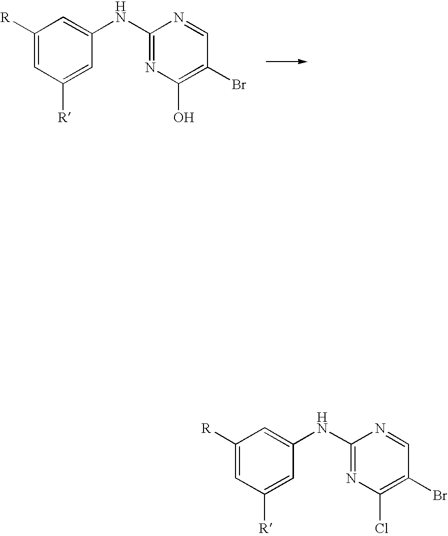

A solution of 5-bromo-2-[arylamino]pyrimidin-4-ol (Intermediate 211 or Intermediate 212, 1 eq) in POCl₃ (15 eq) was heated to reflux for 1 h. The mixture was cooled to RT and POCl₃ was removed in vacuo. To the residue obtained, was added crushed ice with stirring, and the pH was adjusted to 7-8 using 10% sodium bicarbonate solution. The solid obtained was filtered and washed with chilled water to yield the title compound. The compounds in the below table were prepared using this method and the specified starting material.

| Compound | Structure | Mass spectrum and ¹H NMR | SM |
|---|---|---|---|
| Intermediate 213 | 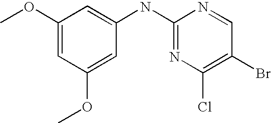 5-bromo-4-chloro-N-(3,5- dimethoxyphenyl)pyrimidin-2- amine | MS (ES): 344 (M), 346 (M + 2) for C₁₂H₁₁BrClN₃O_{2.} 300 MHz, DMSO-d₆: δ 3.77 (s, 6H), 6.19 (t, J = 2.13 Hz, 1H), 6.94 (d, J = 2.19 Hz, 2H), 8.68 (s, 1H), 10.13 (s, 1H). | Intermediate 211 5-bromo-2-[(3,5- dimethoxyphenyl)- amino]- pyrimidin-4- ol |
| Intermediate 214 | 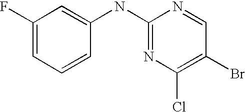 5-bromo-4-chloro-N-(3- fluorophenyl)pyrimidin-2- amine | MS(ES): 302 (M) and 304 (M + 2) for C₁₀H₆BrClFN₃. 400 MHz, DMSO-d₆: δ 6.84 (t, J = 8.40 Hz, 1H), 7.33-7.37 (m, 1H), 7.42 (d, J = 8.48 Hz, 1H), 7.66 (d, J = 12.08 Hz, 1H), 8.74 (s, 1H), 10.43 (s, 1H). | Intermediate 212 5-bromo-2-[(3- fluorophenyl)- amino]- pyrimidin-4-ol |

### General procedure for 5-bromo-N-(aryl)-4-[3-(trifluoromethyl)-1H-pyrazol-1-yl]pyrimidin-2-amine and 5-bromo-N-(aryl)-4-[5-methyl-3-(trifluoromethyl)-1H-pyrazol-1-yl]pyrimidin-2-amine

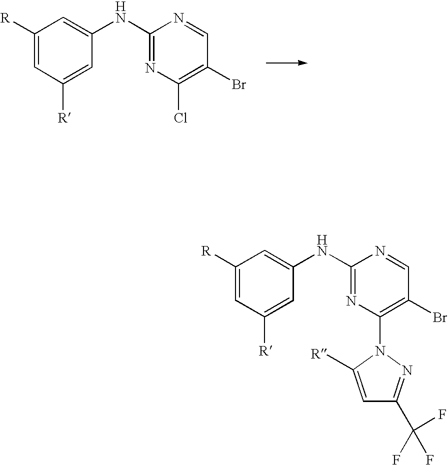

NaH (1.2 eq) was dissolved in 1 mL of NMP and stirred for about 5 min at 0° C. Then either 3-(trifluoromethyl)-1H-pyrazole or 5-methyl-3-(trifluoromethyl)-1H-pyrazole (1.1 eq) in NMP was added dropwise at 0° C. for about 10 min and stirring continued for about 20 min under N₂. Then the 5-bromo-4-chloro-N-(aryl)pyrimidin-2-amine starting material in NMP was added dropwise and the reaction was stirred at room temperature overnight. After completion of the reaction, water was added and the solid obtained was filtered, dried and purified by silica gel column chromatography using ethyl acetate/hexanes as eluent to yield the desired product. The compounds in the below table were prepared using this method and the specified starting material.

| Compound | Structure | Mass spectrum and ¹H NMR | SM |
|---|---|---|---|
| Intermediate 215 | 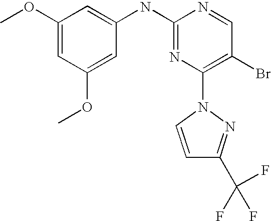 5-bromo-N-(3,5-dimethoxyphenyl)- 4-[3-(trifluoromethyl)- 1H-pyrazol-1-yl]pyrimidin-2- amine | MS(ES): 444 (M) and 446 (M + 2) for C₁₆H₁₃BrF₃N₅O₂. 300 MHz, DMSO-d₆: δ 3.71 (s, 6H), 6.18-6.19 (m, 1H), 7.01 (d, J = 2.13 Hz, 2H), 7.13-7.14 (m, 1H), 8.62 (br s, 1H), 8.88 (br s, 1H), 10.19 (br s, 1H). | Intermediate 213 5-bromo-4-chloro- N-(3,5-dimethoxy- phenyl)pyrimidin- 2-amine |
| Intermediate 216 | 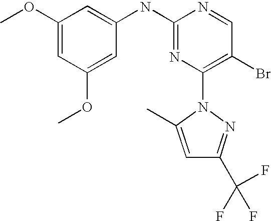 5-bromo-N-(3,5-dimethoxyphenyl)- 4-[5-methyl-3-(trifluoromethyl)- 1H-pyrazol-1-yl]pyrimidin-2- amine | MS(ES): 458 (M) and 460 (M + 2) for C₁₇H₁₅BrF₃N₅O₂. 400 MHz, DMSO-d₆: δ 2.38 (s, 3H), 3.71 (s, 6H), 6.20 (t, J = 2.80 Hz, 1H), 6.84 (s, 1H),6.93 (d, J = 2.84 Hz, 2H), 8.91 (s, 1H), 10.21 (br s, 1H). | Intermediate 213 |
| Intermediate 217 | 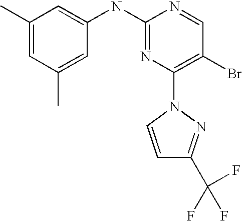 5-bromo-N-(3,5-dimethylphenyl)- 4-[3-(trifluoromethyl)- 1H-pyrazol-1-yl]pyrimidin-2- amine | Taken to the next step based on LCMS without further purification. MS(ES): 412 (M) and 414 (M + 2) for C₁₆H₁₃BrF₃N₅. (80% pure by LCMS). | Intermediate 143 5-bromo-4-chloro- N-(3,5- dimethylphenyl)- pyrimidin- 2-amine |
| Intermediate 218 | 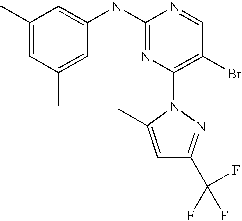 5-bromo-N-(3,5-dimethylphenyl)- 4-[5-methyl-3-(trifluoromethyl)- 1H-pyrazol-1-yl]pyrimidin-2- amine | Taken to the next step based on LCMS without further purification MS(ES): 426 (M) and 428 (M + 2) for C₁₇H₁₅BrF₃N₅. (61% pure by LCMS). | Intermediate 143 5-bromo-4-chloro- N-(3,5- dimethylphenyl)- pyrimidin- 2-amine |
| Intermediate 219 | 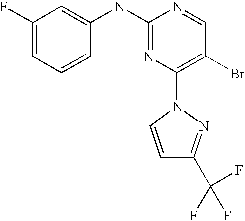 5-bromo-N-(3-fluorophenyl)-4- [3-(trifluoromethyl)-1H-pyrazol-1- yl]pyrimidin-2-amine | MS(ES): 402 (M) and 404 (M + 2) for C₁₄H₈BrF₄N₅. 400 MHz, DMSO-d₆: δ 6.84 (td, J = 2.24, 8.34 Hz, 1H), 7.15 (d, J = 2.64 Hz, 1H), 7.35 (dd, J = 8.12, 15.20 Hz, 1H), 7.47 (d, J = 8.24 Hz, 1H), 7.70 (d, J = 12.08 Hz, 1H), 8.64 (br s, 1H), 8.93 (s, 1H), 10.46 (s, 1H). | Intermediate 214 5-bromo-4-chloro- N-(3-fluorophenyl) pyrimidin-2- amine |
| Intermediate 220 | 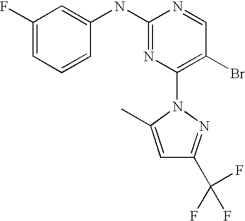 5-bromo-N-(3-fluorophenyl)-4- [5-methyl-3-(trifluoromethyl)- 1H-pyrazol-1-yl]pyrimidin-2- amine | MS(ES): 416 (M) and 418 (M + 2) for C₁₅H₁₀BrF₄N₅. 400 MHz, DMSO-d₆: δ 2.40 (s, 3H), 6.83-6.87 (m, 2H), 7.35 (dd, J = 8.40, 15.20 Hz, 1H), 7.44 (d, J = 8.40 Hz, 1H), 7.65 (d, J = 12.00 Hz, 1H), 8.98 (s, 1H), 10.49 (s, 1H). | Intermediate 214 5-bromo-4-chloro- N-(3-fluorophenyl) pyrimidin-2- amine |

### Intermediate 221: 5-bromo-4-(methylsulfanyl)-N-[3-(methylsulfonyl)phenyl]pyrimidin-2-amine

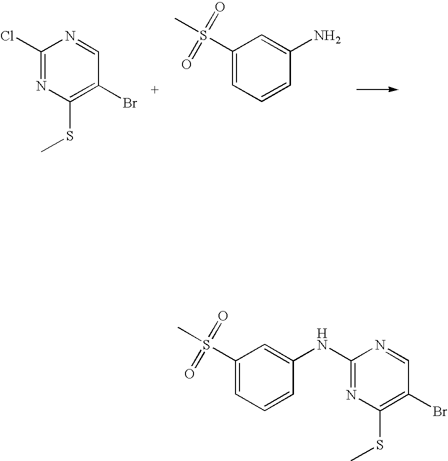

To 6 g of 5-bromo-2-chloro-4-(methylsulfanyl)pyrimidine (25.1 mmol) suspended in n-BuOH (20 mL), was added 3-(methylsulfonyl)aniline hydrochloride (25.1 mmol, 5.2 g) followed by HCl in dioxane (25 mL) and refluxed at 100° C. for 3 h. The reaction was cooled to room temperature, diluted with diethyl ether and the solid was filtered and dried to give the title compound (3.1 g).

| Compound | Structure | Mass spectrum and ¹H NMR | SM |
|---|---|---|---|
| Intermediate 221 | 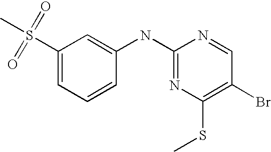 5-bromo-4-(methylsulfanyl)- N-[3-(methylsulfonyl) phenyl]pyrimidin-2-amine | MS(ES): 374 (M) and 376 (M + 2) for C₁₂H₁₂BrN₃O₂S₂. 400 MHz, DMSO-d₆: δ 2.61 (s, 3H), 3.33 (s, 3H), 7.51-7.53 (m, 1H), 7.57 (t, J = 7.60 Hz, 1H), 7.85 (d, J = 8.00 Hz, 1H), 8.38 (s, 1H), 8.57 (s, 1H), 10.21 (br s, 1H). | 3-(methylsulfonyl)- aniline hydrochloride and 5-bromo-2- chloro-4- (methylsulfanyl)- pyrimidine |

### Intermediate 222: 5-bromo-4-(methylsulfonyl)-N-[3-(methylsulfonyl)phenyl]pyrimidin-2-amine

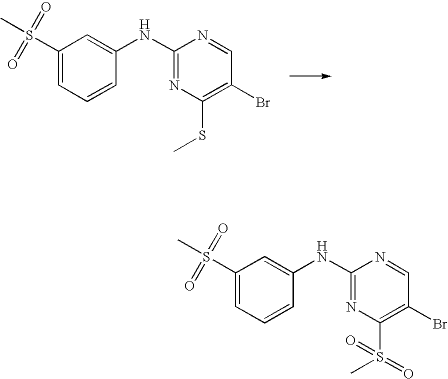

A suspension of 5-bromo-4-(methylsulfanyl)-N-[3-(methylsulfonyl)phenyl]pyrimidin-2-amine (Intermediate 221, 8.02 mmol, 3 g) in dichloromethane (125 mL) was cooled to 0° C. and 3-chloroperoxybenzoic acid (77%, 27.72 mmol, 6.22 g) was added portion wise. The suspension became a clear solution after stirring at 0° C. for 30 min The reaction mixture was then allowed to warm up slowly to room temperature and stirred for 5 h. The pH of the reaction mixture was raised to 8 with the addition of 10% aq. NaHCO₃ solution (50 mL), extracted with dichloromethane (3×10 mL) and the combined organic extracts were washed with brine, dried over Na₂SO₄, filtered and concentrated to give the title compound (1.78 g).

| Compound | Structure | Mass spectrum and ¹H NMR | SM |
|---|---|---|---|
| Intermediate 222 | 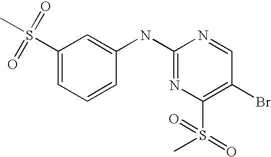 5-bromo-4-(methylsulfonyl)- N-[3-(methylsulfonyl) phenyl]pyrimidin-2-amine | MS(ES): 406 (M) and 408 (M + 2) for C₁₂H₁₂BrN₃O₄S₂. 400 MHz, DMSO-d₆: δ 3.21 (s, 3H), 3.50 (s, 3H), 7.58-7.63 (m, 2H), 7.83 (d, J = 6.08 Hz, 1H), 8.41 (br s, 1H), 8.97 (s, 1H), 10.75 (br s, 1H). | Intermediate 221 5-bromo-4- (methylsulfanyl)- N-[3- (methylsulfonyl)- phenyl]pyrimidin-2- amine |

### Intermediate 223: ethyl(2E)-3-(3-{2-[(3,5-difluorophenyl)amino]-4-(methylsulfonyl)pyrimidin-5-yl}phenyl)prop-2-enoate

A suspension of 5-bromo-N-(3,5-difluorophenyl)-4-(methylsulfonyl)pyrimidin-2-amine Intermediate 139 (0.85 mmol, 0.3 g), 134(1E)-3-ethoxy-3-oxoprop-1-en-1-yl]phenyl}boronic acid (0.93 mmol, 0.206 g), bis(diphenylphosphino)ferrocene]dichloropalladium(II) complex with CH₂Cl₂ (0.14 mmol, 0.112 g) and sodium carbonate (1.27 mmol, 0.14 g) in acetonitrile/water (5 mL:2 mL) was heated to 90° C. for 30 minutes. The reaction mixture was concentrated in vacuo and the residue taken in ethyl acetate (50 mL) was washed with water and brine. The organic layer was dried over sodium sulfate, filtered and concentrated. The crude compound was purified by silica gel column chromatography using 50% ethyl acetate/hexanes as an eluent to yield the title compound (300 mg).

| | | Mass spectrum and | |
|---|---|---|---|
| Compound | Structure | ¹H NMR | SM |
| Intermediate 223 | 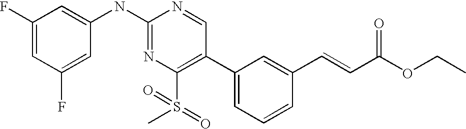 ethyl (2E)-3-(3-{2-[(3,5-difluorophenyl)amino]-4- (methylsulfonyl)pyrimidin-5-yl}phenyl)prop-2-enoate | Taken to the next step based on LCMS without further purification. MS(ES): 460 (M + 1) for C₂₂H₁₉F₂N₃O₄S. (94% pure by LCMS) | Intermediate 139 5-bromo-N-(3,5- difluorophenyl)-4- (methylsulfonyl) pyrimidin- 2-amine |

### Intermediate 224: ethyl(2E)-3-{3-[4-(methylsulfonyl)-2-{[3-(methylsulfonyl)phenyl]amino}pyrimidin-5-yl]phenyl}prop-2-enoate

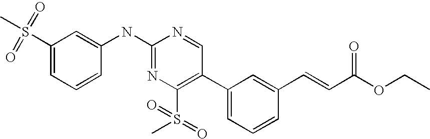

A suspension of 5-bromo-4-(methylsulfonyl)-N-[3-(methylsulfonyl)phenyl]pyrimidin-2-amine Intermediate 222 (3.69 mmol, 1.5 g), {3-[(1E)-3-ethoxy-3-oxoprop-1-en-1-yl]phenyl}boronic acid (4.06 mmol, 0.89 g), bis(diphenylphosphino)ferrocene]dichloropalladium(II) complex with CH₂Cl₂ (0.41 mmol, 0.34 g), 2-dicyclohexylphosphino-2′,4′,6′-triisopropyl-1,1′-biphenyl (1.11 mmol, 0.53 g) and sodium carbonate (4.43 mmol, 0.47 g) in acetonitrile/water (5:1) was heated to 90° C. for 30 minutes. The reaction mixture was diluted with ethyl acetate (50 mL) and the organic layer was separated, dried over sodium sulfate, filtered and concentrated. The crude compound was purified by silica gel column chromatography using EtOAc/hexanes as an eluent to yield the title compound (1.02 g).

| | | Mass spectrum and | |
|---|---|---|---|
| Compound | Structure | ¹H NMR | SM |
| Intermediate 224 | 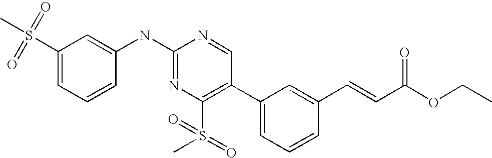 ethyl (2E)-3-{3-[4-(methylsulfonyl)-2-{[3- (methylsulfonyl)phenyl]amino}pyrimidin-5- yl]phenyl}prop-2-enoate | MS(ES): 502 (M + 1) for C₂₃H₂₃N₃O₆S₂. 300 MHz, DMSO-d₆: δ 1.25 (t, J = 87.08 Hz, 3H), 3.21 (s, 3H), 3.41 (s, 3H), 4.19 (q, J = 7.05 Hz, 2H), 6.68 (d, J = 16.02 Hz, 1H), 7.50-7.71 (m, 6H), 7.85-7.92 (m, 2H), 8.50 (br s, 1H), 8.83 (s, 1H), 10.74 (br s, 1H). | Intermediate 222 5-bromo-4- (methylsulfonyl)- N-[3- (methylsulfonyl)- phenyl]pyrimidine- 2-amine |

### Intermediate 225: ethyl 5-{2-[(3,5-difluorophenyl)amino]-4-(methylsulfonyl)pyrimidin-5-yl}pyridine-3-carboxylate

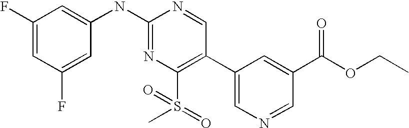

A suspension of 5-bromo-N-(3,5-difluorophenyl)-4-(methylsulfonyl)pyrimidin-2-amine Intermediate 139 (1.38 mmol, 0.5 g), ethyl 5-(4,4,5,5-tetramethyl-1,3,2-dioxaborolan-2-yl)pyridine-3-carboxylate (1.65 mmol, 0.46 g), bis(diphenylphosphino)ferrocene]dichloropalladium(II) complex with CH₂Cl₂ (0.14 mmol, 0.112 g) and sodium carbonate (1.37 mmol, 0.146 g) in acetonitrile/water (10 mL:3 mL) was heated to 90° C. for 30 minutes. The reaction mixture was concentrated in vacuo and the residue taken in ethyl acetate (50 mL) was washed with water and brine. The organic layer was dried over sodium sulfate, filtered and concentrated. The crude compound was purified by silica gel column chromatography using 50% ethyl acetate/hexanes as an eluent to yield the title compound (400 mg).

| | | Mass spectrum and ¹H | |
|---|---|---|---|
| Compound | Structure | NMR | SM |
| Intermediate 225 | 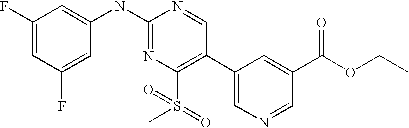 ethyl 5-{2-[(3,5-difluorophenyl)amino]-4- (methylsulfonyl)pyrimidin-5-yl}pyridine-3- carboxylate | MS(ES): 435 (M + 1) for C₁₉H₁₆F₂N₄O₄S. 400 MHz, DMSO-d₆: δ 1.35 (t, J = 7.20 Hz, 3H), 3.40 (s, 3H), 4.39 (q, J = 7.20 Hz, 2H), 6.89-6.93 (m, 1H), 7.52 (dd, J = 2.40, 10.00 Hz, 2H), 8.42 (t, J = 2.00 Hz, 1H), 8.67 (d, J = 2.40 Hz, 1H), 8.67 (s, 1H), 9.13 (s, 1H), 10.81 (s, 1H). | Intermediate 139 5-bromo-N-(3,5- difluorophenyl)-4- (methylsulfonyl)- pyrimidin- 2-amine |

### Intermediate 226: ethyl 5-[4-(methylsulfonyl)-2-{[3-(methylsulfonyl)phenyl]amino}pyrimidin-5-yl]pyridine-3-carboxylate

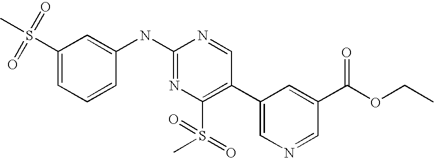

A suspension of 5-bromo-4-(methylsulfonyl)-N-[3-(methylsulfonyl)phenyl]pyrimidin-2-amine Intermediate 224 (3.94 mmol, 1.6 g), ethyl 5-(4,4,5,5-tetramethyl-1,3,2-dioxaborolan-2-yl)pyridine-3-carboxylate (4.33 mmol, 1.2 g), bis(diphenylphosphino)ferrocene]dichloropalladium(H) complex with CH₂Cl₂ (0.44 mmol, 0.36 g), 2-dicyclohexylphosphino-2′,4′,6′-triisopropyl-1,1′-biphenyl (1.12 mmol, 0.56 g,) and sodium carbonate (4.72 mmol, 0.5 g) in acetonitrile/water (5:1) was heated to 90° C. for 30 minutes. The reaction mixture was diluted with ethyl acetate (50 mL) and the organic layer was separated, dried over sodium sulfate, filtered and concentrated. The crude compound was purified by silica gel column chromatography using EtOAc/hexanes as an eluent to yield the title compound (1.05 g).

| | | Mass spectrum and ¹H | |
|---|---|---|---|
| Compound | Structure | NMR | SM |
| Intermediate 226 | 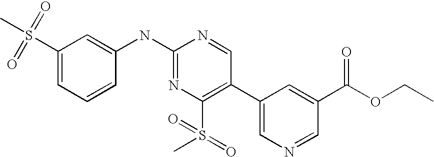 ethyl 5-[4-(methylsulfonyl)-2-{[3- (methylsulfonyl)phenyl]amino}pyrimidin-5- yl]pyridine-3-carboxylate | MS(ES): 477 (M + 1) for C₂₀H₂₀N₄O₆S₂. 400 MHz, DMSO-d₆: δ 1.34 (t, J = 7.08 Hz, 3H), 3.22 (s, 3H), 3.44 (s, 3H), 4.38 (q, J = 7.12 Hz, 2H), 7.60-7.62 (m, 1H), 7.65 (t, J = 7.84 Hz, 1H), 7.90 (d, J = 7.92 Hz, 1H), 8.42 (t, J = 2.12 Hz, 1H), 8.52 (br s, 1H), 8.87 (s, 1H), 8.89 (d, J = 2.16 Hz, 1H), 9.12 (d, J = 2.00 Hz, 1H), 10.83 (br s, 1H). | Intermediate 224 5-bromo-4- (methylsulfonyl)-N- [3-(methylsulfonyl) phenyl]pyrimidin-2- amine |

### Intermediate 227: 5-bromo-2-chloro-4-[3-(trifluoromethyl)-1H-pyrazol-1-yl]pyrimidine

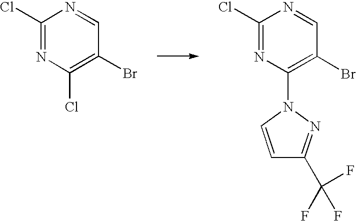

To a suspension of K₂CO₃ (8.8 mmol, 1.2 g) in acetonitrile (25 mL), was added 5-bromo-2,4-dichloropyrimidine (8.8 mmol, 2 g) and the reaction mixture was cooled to −5 to −10° C. 3-(trifluoromethyl)-1H-pyrazole (8.8 mmol, 1.2 g) was dissolved in 25 mL of acetonitrile and added dropwise. After the addition, the reaction mixture was slowly warmed to RT and stirred for 5 h. The reaction mixture was filtered through a celite bed and acetonitrile was removed in vacuo. The crude mass was purified by silica gel column chromatography (60-120 mesh; product eluted with 2% EtOAc/hexanes) to yield 1.4 g of the product.

| Compound | Structure | Mass spectrum and ¹H NMR | SM |
|---|---|---|---|
| Intermediate 227 | 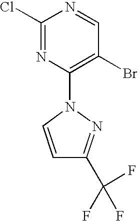 | MS(ES): 327 (M) and 329 (M + 2) for C₈H₃BrClF₃N₄. 300 MHz, CDCl₃: δ 6.81 (d, J = 2.73 Hz, 1H), 8.57 (d, J = 1.98 Hz, 1H), 8.91 (s, 1H). | 5-bromo-2,4- dichloro- pyrimidine and 3- (trifluoro- methyl)-1H- pyrazole |

### Intermediate 228: 5-bromo-N-(3,5-dimethylphenyl)-4-[3-(trifluoromethyl)-1H-pyrazol-1-yl]pyrimidin-2-amine

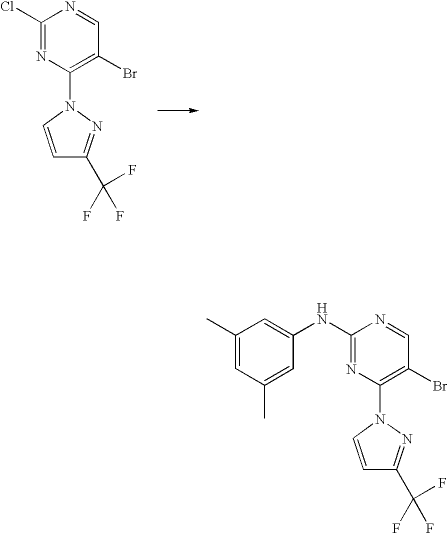

To a suspension of Cs₂CO₃ (2.4 mmol, 780 mg) in DMF (4 mL), were added 5-bromo-2-chloro-4-[3-(trifluoromethyl)-1H-pyrazol-1-yl]pyrimidine (Intermediate 227, 1.2 mmol, 400 mg) and 3,5-dimethylaniline (1.4 mmol, 177 mg) and heated to 100° C. in a sealed tube for 3 h. The reaction mixture was then diluted with EtOAc and passed through a celite bed. The organic layer was washed with brine and dried over sodium sulphate. It was further concentrated and the crude mass was purified by silica gel column chromatography (60-120 mesh; product eluted with 5% EtOAc/hexanes) to yield 330 mg of 5-bromo-N-(3,5-dimethylphenyl)-4-[3-(trifluoromethyl)-1H-pyrazol-1-yl]pyrimidin-2-amine.

| Compound | Structure | Mass spectrum and ¹H NMR | SM |
|---|---|---|---|
| Intermediate 228 | 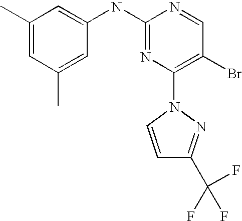 5-bromo-N-(3,5-dimethylphenyl)- 4-[3-(trifluoromethyl)- 1H-pyrazol-1-yl]pyrimidin-2- amine | Taken to the next step based on LCMS without further purification. MS(ES): 412 (M) and 414 (M + 2) for C₁₆H₁₃BrF₃N₅. (80% pure by LCMS). | Intermediate 227 5-bromo-2- chloro-4-[3- (trifluoromethyl)- 1H-pyrazol-1- yl]pyrimidine |

### General procedures for the synthesis of 5-bromo-N-(aryl)-4-methoxypyrimidin-2-amine

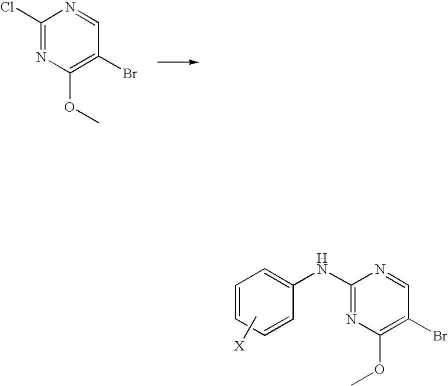

### Method A

To a stirred solution of 5-bromo-2-chloro-4-methoxypyrimidine (1 eq) and arylamine (1.02 eq) in n-BuOH (12 v/w) was added dioxane-HCl (1 v/w) slowly. The reaction mixture was heated to 110° C. for 3 h. It was then cooled to room temperature, diethyl ether (150 mL) was added and the resulting solid was filtered to yield the product.

### Method B

To a stirred solution of 5-bromo-2-chloro-4-methoxypyrimidine (1.3-1.5 eq) and arylamine (1 eq) in 2,2,2-trifluoroethanol (10 v/w), trifluoroacetic acid (2-4 eq) was added and the solution was refluxed at 75° C. for 2-12 h with constant stirring. Water was added to the reaction mixture and extracted with EtOAc (2×50 mL). The organic layer was washed with water, 10% NaHCO₃ solution and brine, dried over sodium sulfate and concentrated in vacuo to yield the product. The compounds in the below table were prepared using the above methods as indicated and the starting material specified.

| Compound | Structure | Mass spectrum and ¹H NMR | SM |
|---|---|---|---|
| Intermediate 229^{b)} | 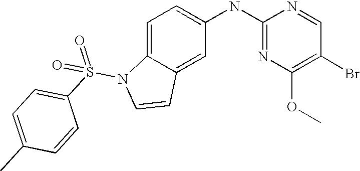 N-(5-bromo-4-methoxypyrimidin-2-yl)-1-[(4- methylphenyl)sulfonyl]-1H-indol-5-amine | MS(ES): 473 (M) and and 475 (M + 2) for C₂₀H₁₇BrN₄O₃S. 300 MHz, DMSO-d₆: δ 2.29 (s, 3H), 3.98 (s, 3H), 6.78 (d, J = 3.60 Hz, 1H), 7.36 (d, J = 8.04 Hz, 2H), 7.58 (d, 1H), 7.71 (d, J = 3.54 Hz, 1H), 7.81-7.84 (m, 3H), 7.99 (s, 1H), 8.33 (d, J = 1.11 Hz, 1H), 9.77 (s, 1H). | 5-Amino-4- toluenesulphonyl)- 1H-indole hydrochloride |
| Intermediate 230^{a)} | 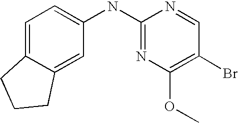 5-bromo-N-(2,3-dihydro-1H- inden-5-yl)-4-methoxypyrimidin-2-amine | MS(ES): 320 (M) and 322 (M + 2) for C₁₄H₁₄BrN₃O 400 MHz, DMSO-d₆: δ 1.98-2.02 (m, 2H), 2.78-2.85 (m, 4H), 3.98 (s, 3H), 7.13 (d, J = 8.16 Hz, 1H), 7.41 (dd, J = 1.72, 8.12 Hz, 1H), 7.58 (s, 1H), 8.35 (s, 1H), 9.74 (s, 1H). | 5-Aminoindane |
| Intermediate 231^{b)} | 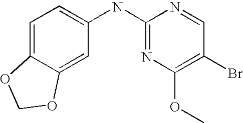 N-(1,3-benzodioxol-5- yl)-5-bromo-4-methoxypyrimidin-2-amine | MS(ES): 324 (M) and 326 (M + 2) for C₁₂H₁₀BrN₃O₃ 300 MHz, DMSO-d₆: δ 3.95 (s, 3H), 5.95 (s, 2H), 6.85 (d, J = 2.73 Hz, 1H), 7.10 (d, J = 2.85 Hz, 1H), 7.39 (d, J = 1.92 Hz, 1H), 8.31 (s, 1H), 9.60 (s, 1H). | 3,4-Methylenedioxy- aniline |
| Intermediate 232^{b)} | 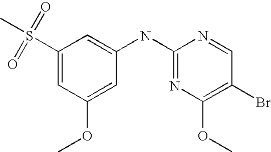 5-bromo-4-methoxy-N-[3- methoxy-5-(methylsulfonyl) phenyl]pyrimidin-2-amine | Taken to the next step based on LCMS without further purification. MS(ES): 388 (M) and 390 (M + 2) for C₁₃H₁₄BrN₃O₄S. | 3-methoxy-5- (methylsulfonyl)- aniline |

^{a)}Method A
^{b)}Method B

### General procedures for the Synthesis of 5-bromo-2-[arylamino]pyrimidin-4-ol

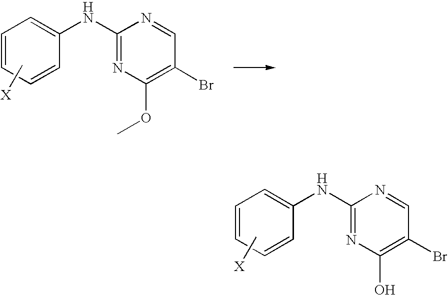

### Method C

A mixture of the 4-methoxypyrimidine derivative (1 eq) and aq. sodium thiomethoxide (21% w/v, 4 eq) and DMF (8 ml/g SM) was heated to 60° C. for 2 h; cooled to room temperature, poured into water (150 mL) and acidified with 1.5 N HCl solution. The precipitated solid was filtered to yield the product.

### Method D

A solution of the 4-methoxypyrimidine derivative (1 g) in n-BuOH (7 ml/g SM) and 4M HCl in dioxane (4 ml/g SM) were heated to 110° C. in a sealed tube and stirred for 18-36 h. The reaction mixture was cooled to room temperature and BuOH was concentrated under vacuum. To the residue obtained, was added water (50 mL) and neutralized using 10% NaHCO₃ solution. The resulting solid was filtered and further purified by column chromatography using ethyl acetate/hexanes as eluent to yield the product.

The compounds in the below table were prepared using the above methods as indicated and the starting material specified.

| Compound | Structure | Mass spectrum and ¹H NMR | SM |
|---|---|---|---|
| Intermediate 233^{d)} | 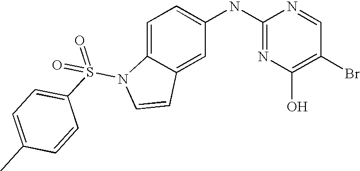 5-bromo-2-({1-[(4-methylphenyl)- sulfonyl]-1H-indol-5-yl}amino)- pyrimidin-4-ol | Taken to the next step based on LCMS without further purification. MS(ES): 459 (M) and and 461 (M + 2) for C₁₉H₁₅BrN₄O₃S. (65% pure by LCMS). | Intermediate 229 N-(5-bromo-4- methoxypyrimidin- 2-yl)-1-[(4- methylphenyl)sul- fonyl]-1H-indol-5- amine |
| Intermediate 234^{c)} | 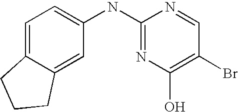 5-bromo-2-(2,3-dihydro-1H-inden-5- ylamino)pyrimidin-4-ol | MS(ES): 306 (M) and 308 (M + 2) for C₁₃H₁₂BrN₃O. 400 MHz, DMSO-d₆: δ 1.97-2.04 (m, 2H), 2.78-2.85 (m, 4H), 7.15 (d, J = 8.08 Hz, 1H), 7.22 (dd, J = 1.40, 8.08 Hz, 1H), 7.44 (s, 1H), 8.01 (s, 1H), 8.87 (s, 1H), 11.21 (br s, 1H). | Intermediate 230 5-bromo-N-(2,3- dihydro-1H-inden- 5-yl)-4- methoxypyrimidin- 2-amine |
| Intermediate 235^{c)} | 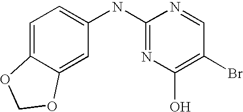 2-(1,3-benzodioxol-5- ylamino)-5-bromopyrimidin-4-ol | Taken to the next step based on LCMS without further purification. MS(ES): 311 (M + 1) for C1₁H₈BrN₃O₃ | Intermediate 231 N-(1,3- benzodioxol-5-yl)- 5-bromo-4- methoxypyrimidin- 2-amine |
| Intermediate 236^{d)} | 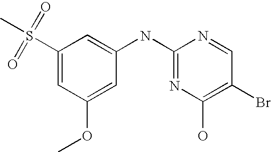 5-bromo-2-{[3-methoxy-5- (methylsulfonyl)phenyl]amino}- pyrimidin-4-ol | Taken to the next step based on LCMS without further purification. MS(ES): 374 (M) and 376 (M + 2) for C₁₂H₁₂BrN₃O₄S. | Intermediate 232 (5-bromo-4- methoxy-N-[3- methoxy-5- (methylsulfonyl)- phenyl]pyrimidin-2- amine |

^{c)}Method C
^{d)}Method D

### General procedure for the synthesis of 5-bromo-4-chloro-N-(aryl)pyrimidin-2-amine

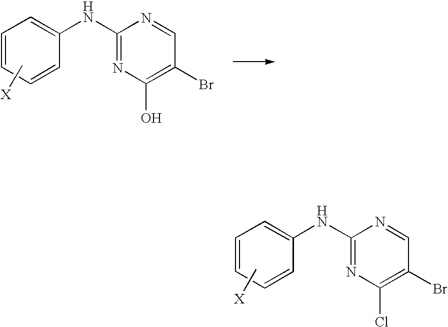

A solution of 5-bromo-2-[arylamino]pyrimidin-4-ol derivative in POCl₃ (3-10 ml/g SM) was heated to reflux for 1 h. It was cooled to RT and POCl₃ was removed in vacuo. To the residue obtained, was added crushed ice with stirring, and the pH was adjusted 7-8 using 10% sodium bicarbonate solution. The solid obtained was filtered and washed with chilled water to yield the title compound. The compounds in the below table were prepared using the above method and the starting material specified.

| Compound | Structure | Mass spectrum and ¹H NMR | SM |
|---|---|---|---|
| Intermediate 237 | 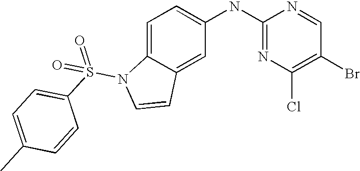 N-(5-bromo-4-chloropyrimidin- 2-yl)-1-[(4-methylphenyl)- sulfonyl]-1H-indol-5-amine | MS(ES): 477 (M) and 479 (M + 2) for C₁₉H₁₄BrClN₄O₂S. 400 MHz, DMSO-d₆: δ 2.31 (s, 3H), 6.82 (d, J = 3.60 Hz, 1H), 7.38 (d, J = 8.28 Hz, 2H), 7.52 (dd, J = 2.00, 9.00 Hz, 1H), 7.76 (d, J = 3.64 Hz, 1H), 7.86 (m, 3H), 7.92 (d, J = 1.88 Hz, 1H), 8.65 (s, 1H), 10.24 (s, 1H). | Intermediate 233 5-bromo-2-({1-[(4- methylphenyl) sulfonyl]-1H-indol-5- yl}amino)pyrimidin- 4-ol |
| Intermediate 238 | 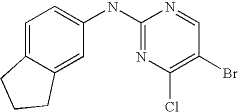 5-bromo-4-chloro-N-(2,3- dihydro-1H-inden-5- yl)pyrimidin-2-amine | MS(ES): 324 (M) and 326 (M + 2) for C₁₃H₁₁BrClN₃. 300 MHz, DMSO-d₆: δ 1.96- 2.04 (m, 2H), 2.76-2.84 (m, 4H), 7.13 (d, J = 8.13 Hz, 1H), 7.35 (dd, J = 1.44, 6.54 Hz, 1H), 7.51 (s, 1H), 8.61 (s, 1H), 10.06 (s, 1H). | Intermediate 234 5-bromo-2-(2,3- dihydro-1H-inden- 5- ylamino)pyrimidin- 4-ol |
| Intermediate 239 | 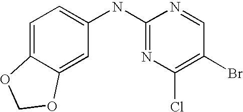 N-(1,3-benzodioxol-5-yl)-5-bromo-4- chloropyrimidin-2-amine | MS(ES): 329 (M + 1) and 330 (M + 2) for C₁₁H₇BrClN₃O₂ 400 MHz, DMSO-d₆: δ 5.98 (s, 2H), 6.87 (d, J = 8.36 Hz, 1H), 7.02 (dd, J = 2.12, 8.44 Hz, 1H), 7.30 (d, J = 2.04 Hz, 1H), 8.72 (s, 1H), 10.06 (s, 1H). | Intermediate 235 2-(1,3- benzodioxol-5- ylamino)-5- bromopyrimidin-4- ol |
| Intermediate 240^{e)} | 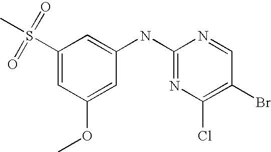 5-bromo-4-chloro-N-[3- methoxy-5-(methylsulfonyl)- phenyl]pyrimidin-2-amine | Taken to the next step based on LCMS without further purification. MS(ES): 394 (M + 2) for C₁₂H₁₁BrClN₃O₃S. | Intermediate 236 5-bromo-2-{[3- methoxy-5- (methylsulfonyl)- phenyl]amino} pyrimidin-4-ol |

^{e)}N,Ndiethylaniline (0.5 eq) was also added

### General procedure for the synthesis of 5-bromo-N-(aryl)-4-[3-(trifluoromethyl)-1H-pyrazol-1-yl]pyrimidin-2-amine and 5-bromo-N-(aryl)-4-[5-methyl-3-(trifluoromethyl)-1H-pyrazol-1-yl]pyrimidin-2-amine

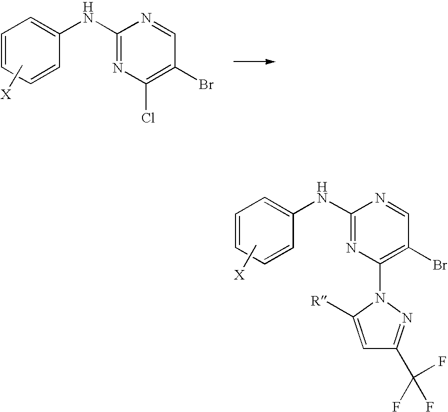

NaH (1.2 eq) was dissolved in 1 mL of NMP and stirred for about 5 min at 0° C. Then either 3-(trifluoromethyl)-1H-pyrazole or 5-methyl-3-(trifluoromethyl)-1H-pyrazole (1.1 eq) in NMP was added dropwise at 0° C. for 10 min and stirring continued for 20 min under N₂. Then the 5-bromo-4-chloro-N-(aryl)pyrimidin-2-amine derivative (1 eq) in NMP was added dropwise and the reaction was stirred at room temperature overnight. After completion of the reaction, water was added and the solid obtained was filtered, dried and purified by silica gel column chromatography using ethyl acetate/hexanes as eluent to yield the desired product. The compounds in the below table were prepared using the above method and the starting material specified.

| Compound | Structure | Mass spectrum and ¹H NMR | SM |
|---|---|---|---|
| Intermediate 241 | 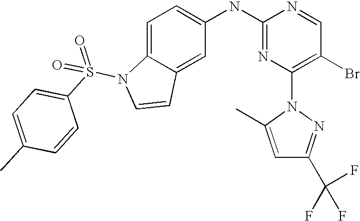 N-{5-bromo-4-[5-methyl-3- (trifluoromethyl)-1H-pyrazol-1- yl]pyrimidin-2-yl}-1-[(4- methylphenyl)sulfonyl]-1H-indol-5- amine | MS(ES): 591 (M) and 593 (M + 2) for C₂₄H₁₈BrF₃N₆O₂S. 400 MHz, DMSO-d₆: δ 2.31 (s, 3H), 2.38 (s, 3H), 6.81 (d, J = 3.64 Hz, 1H), 6.84 (s, 1H), 7.38 (d, J = 8.20 Hz, 2H), 7.54 (dd, J = 2.12, 8.96 Hz, 1H), 7.76 (d, J = 3.64 Hz, 1H), 7.84-7.89 (m, 4H), 8.89 (s, 1H), 10.30 (s, 1H). | Intermediate 237 N-(5-bromo-4- chloropyrimidin- 2-yl)-1-[(4- methylphenyl)- sulfonyl]-1H- indol-5-amine |
| Intermediate 242^{f)} | 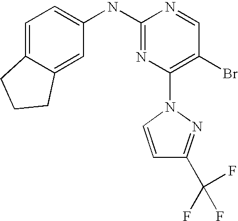 5-bromo-N-(2,3-dihydro-1H-inden- 5-yl)-4-[3-(trifluoromethyl)- 1H-pyrazol-1-yl]pyrimidin-2- amine | MS(ES): 424 (M) and 426 (M + 2) for C₁₇H₁₃BrF₃N₅ 300 MHz, DMSO-d₆: δ 1.97-2.02 (m, 2H), 2.77-2.85 (m, 4H), 7.11 (d, J = 2.55 Hz, 1H), 7.15 (d, J = 8.07 Hz, 1H), 7.39 (d, J = 7.77 Hz, 1H), 7.56 (s, 1H), 8.59 (s, 1H), 8.83 (s, 1H), 10.11 (s, 1H). | Intermediate 238 5-bromo-4- chloro-N-(2,3- dihydro-1H- inden-5- yl)pyrimidin- 2-amine |
| Intermediate 243^{f)} | 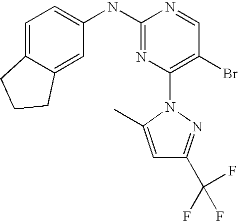 5-bromo-N-(2,3-dihydro-1H-inden- 5-yl)-4-[5-methyl-3- (trifluoromethyl)-1H-pyrazol-1- yl]pyrimidin-2-amine | MS(ES): 438 (M) and 440 (M + 2) for C₁₈H₁₅BrF₃N₅. 400 MHz, DMSO-d₆: δ 1.97-2.04 (m, 2H), 2.28 (s, 3H), 2.79-2.85 (m, 4H), 6.84 (s, 1H), 7.16 (d, J = 8.16 Hz, 1H), 7.37 (d, J = 8.08 Hz, 1H), 7.52 (s, 1H), 8.88 (s, 1H), 10.15 (s, 1H). | Intermediate 238 5-bromo-4- chloro-N-(2,3- dihydro-1H- inden-5- yl)pyrimidin- 2-amine |
| Intermediate 244 | 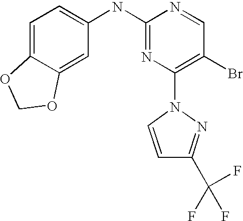 N-(1,3-benzodioxol-5-yl)- 5-bromo-4-[3-(trifluoromethyl)- 1H-pyrazol-1-yl]pyrimidin-2- amine | MS(ES): 429 (M + 1) for C₁₅H₉BrF₃N₅O₂ 400 MHz, DMSO-d₆: δ 5.99 (s, 2H), 6.88 (d, J = 8.36 Hz, 1H), 7.07 (dd, J = 2.04, 8.42 Hz, 1H), 7.13 (d, J = 2.64 Hz, 1H), 7.37 (d, J = 1.96 Hz, 1H), 8.60 (d, J = 1.60 Hz, 1H), 8.83 (s, 1H), 10.12 (s, 1H). | Intermediate 239 N-(1,3- benzodioxol- 5-yl)-5- bromo-4- chloropyrimidin- 2-amine |
| Intermediate 245 | 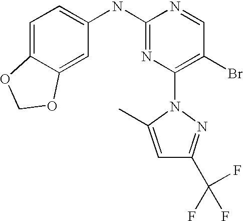 N-(1,3-benzodioxol-5-yl)- 5-bromo-4-[5-methyl-3- (trifluoromethyl)-1H-pyrazol-1- yl]pyrimidin-2-amine | MS(ES): 443 (M + 1) for C₁₆H₁₁BrF₃N₅O₂ 300 MHz, DMSO-d₆: δ 2.36 (s, 3H), 5.97 (s, 2H), 6.81 (s, 1H), 6.86 (d, J = 8.34 Hz, 1H), 7.01 (t, J = 1.98 Hz, 1H), 7.27 (s, 1H), 8.85 (s, 1H), 10.10 (s, 1H). | Intermediate 239 N-(1,3- benzodioxol- 5-yl)-5- bromo-4- chloropyrimidin- 2-amine |
| Intermediate 246^{g)} | 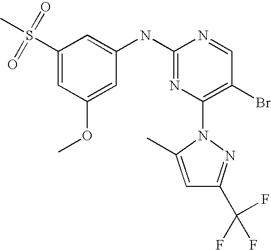 5-bromo-N-[3-methoxy-5- (methylsulfonyl)phenyl]-4-[5- methyl-3-(trifluoromethyl)- 1H-pyrazol-1-yl]pyrimidin-2- amine | MS(ES): 507 (M + 1) and 508 (M + 2) for C₁₇H₁₅BrF₃N₅O₃S. 400 MHz, DMSO-d₆: δ 2.42 (s, 3H), 3.19 (s, 3H), 3.82 (s, 3H), 6.86 (s, 1H), 7.10 (t, J = 1.72 Hz, 1H), 7.62 (s, 1H), 7.88 (s, 1H), 9.01 (s, 1H), 10.60 (s, 1H). | Intermediate 240 5-bromo-4- chloro-N-[3- methoxy-5- (methylsulfonyl) phenyl]- pyrimidin-2- amine |

^{f)}DMSO was used as solvent, 12 h
^{g)}THF was used as solvent, reflux, 7 h

### Intermediate 247: 5-bromo-N-[3-methoxy-5-(methylsulfonyl)phenyl]-4-(methylsulfanyl)pyrimidin-2-amine

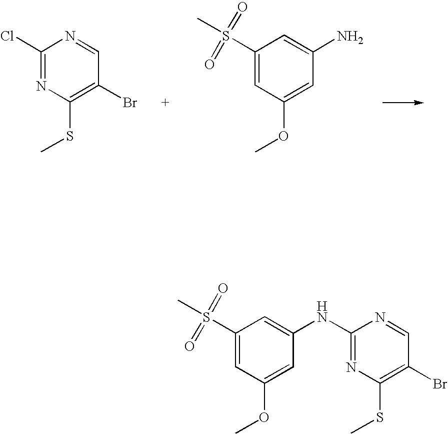

To 6 g of 5-bromo-2-chloro-4-(methylsulfanyl)pyrimidine (1.5 eq) suspended in n-BuOH (30 v/w), was added 3-methoxy-5-(methylsulfonyl)aniline (1 eq followed by. HCl in dioxane (25 mL) and refluxed at 100° C. for 3 h. The reaction was cooled to room temperature, diluted with diethyl ether and the solid was filtered and dried to get.

| Compound | Structure | Mass spectrum and ¹H NMR | SM |
|---|---|---|---|
| Intermediate 247 | 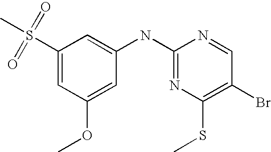 5-bromo-N-[3-methoxy-5- (methylsulfonyl)phenyl]-4- (methylsulfanyl)pyrimidin-2- amine | MS(ES): 404 (M) and 406 (M + 2) for C₁₃H₁₄BrN₃O₃S₂. 300 MHz, DMSO-d₆: δ 2.59 (s, 3H), 3.18 (s, 3H), 3.82 (s, 3H), 7.04 (t, J = 2.07 Hz, 1H), 7.52 (s, 1H), 8.12 (s, 1H), 8.37 (s, 1H), 10.13 (s, 1H). | 3-methoxy-5- (methylsulfonyl)- aniline and 5- bromo-2-chloro- 4-(methylsulfan- yl)pyrimidine |

### Intermediate 248: 5-bromo-N-[3-methoxy-5-(methylsulfonyl)phenyl]-4-(methylsulfonyl)pyrimidin-2-amine (PE-048-017-II)

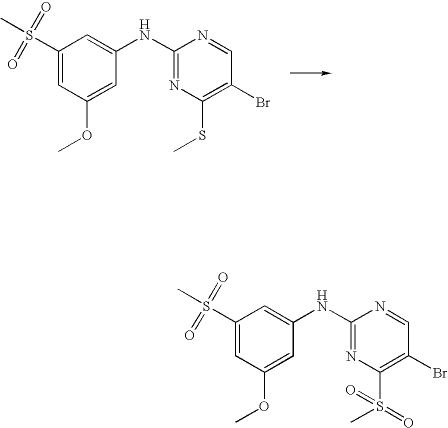

A suspension of 5-bromo-N-[3-methoxy-5-(methylsulfonyl)phenyl]-4-(methylsulfanyl)pyrimidin-2-amine (Intermediate 247, 1 eq) in acetone was cooled to 0° C. and 3-chloroperoxybenzoic acid (77%, 2 eq) was added portion wise. After the completion of the reaction, acetone was removed in vacuo and the residue taken in EtOAc was washed with 10% aq. NaHCO₃ solution (50 mL) and water, dried over Na₂SO₄, filtered and concentrated to give the title compound.

| Compound | Structure | Mass spectrum and ¹H NMR | SM |
|---|---|---|---|
| Intermediate 248 | 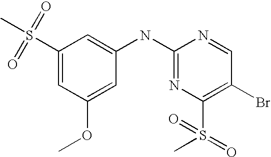 5-bromo-N-[3- methoxy-5- (methylsulfonyl) phenyl]-4- (methylsulfonyl) pyrimidin-2- amine | Taken to the next step based on LCMS without further purification. MS(ES): 436 (M) and 438 (M + 2) for C₁₃H₁₄BrN₃O₅S₂. (72% pure by LCMS) | Intermediate 247 5-bromo-N-[3- methoxy-5- (methylsulfonyl) phenyl]-4- (methylsulfanyl) pyrimidin-2-amine |

### Intermediate 249: ethyl(2E)-3-{3-[2-{[3-methoxy-5-(methylsulfonyl)phenyl]amino}-4-(methylsulfonyl)pyrimidin-5-yl]phenyl}prop-2-enoate

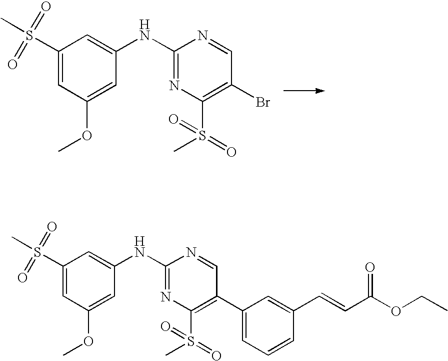

A suspension of 5-bromo-N-[3-methoxy-5-(methylsulfonyl)phenyl]-4-(methylsulfonyl)pyrimidin-2-amine Intermediate 248 (1 eq), {3-[(1E)-3-ethoxy-3-oxoprop-1-en-1-yl]phenyl}boronic acid (1.5 eq), bis(diphenylphosphino)ferrocene]dichloropalladium(II) complex with CH₂Cl₂ (10 mol %) and sodium carbonate (1.5 eq) in acetonitrile/water (10 mL:5 mL) was heated to 90° C. for 30 minutes. The reaction mixture was concentrated in vacuo and the residue taken in ethyl acetate (50 mL) was washed with water and brine. The organic layer was dried over sodium sulfate, filtered and concentrated. The crude compound was purified by silica gel column chromatography using 50% ethyl acetate/hexanes as an eluent to yield Intermediate 249. MS(ES): 532 (M+1) for C₂₄H₂₅N₃O₇S₂.

### Intermediate 250: 5-bromo-N-[3-methoxy-5-(methylsulfonyl)phenyl]-4-[3-(trifluoromethyl)-1H-pyrazol-1-yl]pyrimidin-2-amine

### Intermediate 251: 5-bromo-N-[3-methoxy-5-(methylsulfonyl)phenyl]-4-[5-methyl-3-(trifluoromethyl)-1H-pyrazol-1-yl]pyrimidin-2-amine

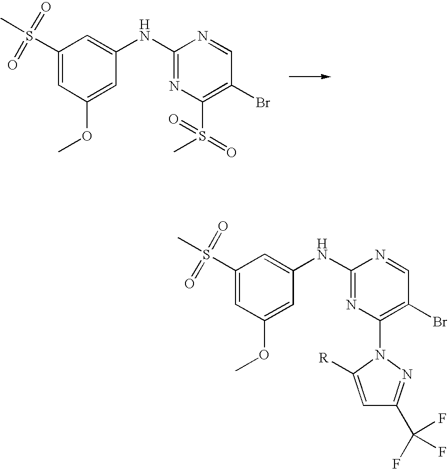

NaH (60% dispersion in mineral oil, 2 eq) was dissolved in 1 mL of DMF and stirred for about 5 min at 0° C. Then 3-(trifluoromethyl)-1H-pyrazole or 5-methyl-3-(trifluoromethyl)-1H-pyrazole (2 eq) in NMP (2 mL) was added dropwise at 0° C. for about 10 min and stirring continued for about 20 min under N₂. Then 5-bromo-N-[3-methoxy-5-(methylsulfonyl)phenyl]-4-(methylsulfonyl)pyrimidin-2-amine (Intermediate 248, 1 eq) in DMF was added dropwise and the reaction was stirred overnight at room temperature. Water was added and the solid obtained was filtered off, dried and purified by silica gel column chromatography using ethyl acetate/hexanes as eluent to yield the product. The compounds in the below table were prepared following this procedure and using the specified starting material.

| Compound | Structure | Mass spectrum and ¹H NMR | SM |
|---|---|---|---|
| Intermediate 250 | 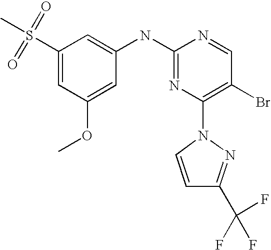 5-bromo-N-[3- methoxy-5- (methylsulfonyl) phenyl]-4-[3- (trifluoromethyl)- 1H-pyrazol-1- yl]pyrimidin-2- amine | Taken to the next step based on LCMS without further purification. MS(ES): 492 (M) and 494 (M + 2) for C₁₆H₁₃BrF₃N₅O₃S. (65% pure by LCMS) | Intermediate 248 5-bromo-N-[3- methoxy-5- (methylsulfonyl) phenyl]-4- (methylsulfonyl) pyrimidin-2-amine |
| Intermediate 251 | 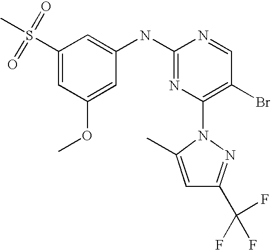 5-bromo-N-[3- methoxy-5- (methylsulfonyl) phenyl]-4-[5- methyl-3- (trifluoromethyl)- 1H-pyrazol-1- yl]pyrimidin-2- amine | Taken to the next step based on LCMS without further purification. MS(ES): 506 (M) and 508 (M + 2) for C₁₇H₁₅BrF₃N₅O₃S. | Intermediate 248 5-bromo-N-[3- methoxy-5- (methylsulfonyl) phenyl]-4- (methylsulfonyl) pyrimidin-2-amine |

### Intermediate 252: 5-bromo-N-(3,5-dimethoxyphenyl)-4-[5-methyl-3-(trifluoromethyl)-1H-pyrazol-1-yl]pyrimidin-2-amine

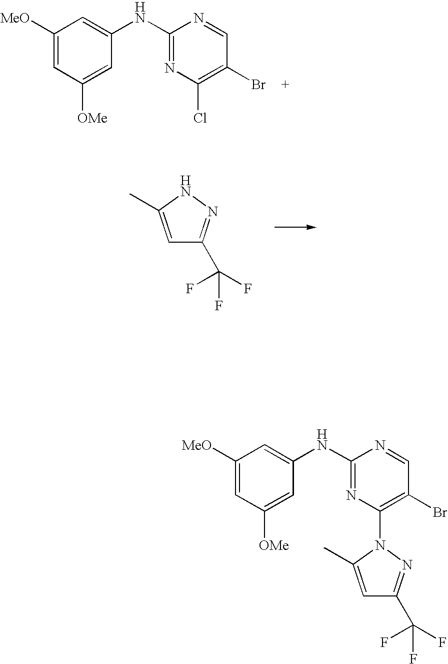

A solution of 3-methyl-5-trifluoromethyl pyrazole (2.87 mmol, 431 mg) in NMP (4 mL) was added slowly to a suspension of sodium hydride (3.13 mmol, 75 mg) in NMP (1 mL) at 0° C. The reaction mixture was stirred for 10 min at 0° C. A solution of 5-bromo-4-chloro-N-(3,5-dimethoxyphenyl)pyridine-2-amine (Intermediate 213, 2.61 mmol, 900 mg) in NMP (4 mL) was added slowly to the reaction mixture at 0° C. and stirred overnight at RT. The reaction mixture was then quenched with ice cold water at 0-5° C., and the pH adjusted to 2 with 1.5 N HCl solution. It was then was extracted with ethyl acetate, and the organic layer was washed with water, dried over Na₂SO₄ and concentrated in vacuo. The crude mass was purified by column chromatography using 10-15% ethyl acetate/hexanes to yield 900 mg of the title compound.

| | | Mass spectrum and ¹H | |
|---|---|---|---|
| Compound | Structure | NMR | SM |
| Intermediate 252 | 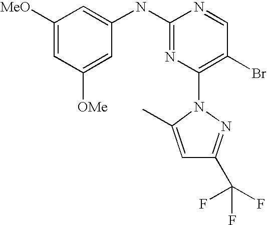 5-bromo-N-(3,5- dimethoxyphenyl)-4- [5-methyl-3- (trifluoromethyl)-1H- pyrazol-1- yl]pyrimidin-2-amine | MS(ES): 458 (M) and 460 (M + 2) for C₁₇H₁₅BrF₃N₅O₂. 400 MHz, DMSO-d₆: δ 2.39 (s, 3H), 3.70 (s, 6H), 6.21 (t, J = 2.20 Hz, 1H), 6.85 (s, 1H), 6.94 (d, J = 2.16 Hz, 2H), 8.93 (s, 1H). | Intermediate 213 5-bromo-4- chloro-N- (3,5- dimethoxy- phenyl) pyrimidin-2- amine |

### Intermediate 253: 1-{3-[(5-bromo-4-hydroxypyrimidin-2-yl)amino]phenyl}ethanone

### Intermediate 254: 5-bromo-2-[(3-methylphenyl)amino]pyrimidin-4-ol

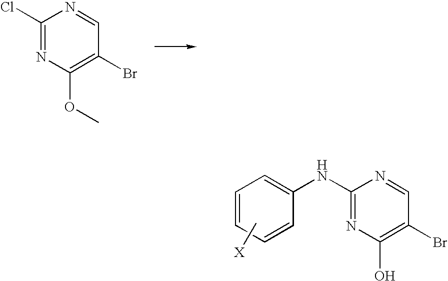

To a stirred solution of 5-bromo-2-chloro-4-methoxypyrimidine (1 eq) and arylamine (1.02 eq) in n-BuOH (12 v/w based on the former), was added dioxane-4M HCl (4 v/w based on the former) and heated to 110° C. in a sealed tube for 18-36 h. The reaction mixture was cooled to room temperature and BuOH was concentrated under vacuum. To the residue obtained, was added water (50 mL) and 10% NaHCO₃ solution. The resulting solid was filtered and further purified by column chromatography to yield the product. The compounds in the below table were prepared using this method and the specified starting material.

| Compound | Structure | Mass spectrum and ¹H NMR | SM |
|---|---|---|---|
| Intermediate 253 | 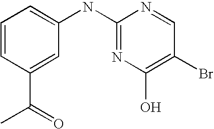 1-{3-[(5- bromo-4- hydroxypyri- midin-2- yl)amino] phenyl} ethanone | MS(ES): 308 (M) and 310 (M + 2) for C₁₂H₁₀BrN₃O₂. Purity by LCMS 97% | 1-(3- aminophenyl) ethanone |
| Intermediate 254 | 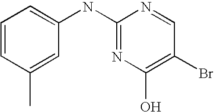 5-bromo-2- [(3- methylphenyl) amino] pyrimidin- 4-ol | MS(ES): 280 (M) and 282 (M + 2) for C₁₁H₁₀BrN₃O. 400 MHz, DMSO-d₆: δ 2.29 (s, 3H), 6.89 (d, J = 7.52 Hz, 1H), 7.19-7.20 (m, 1H), 7.35 (s, 2H), 8.05 (s, 1H), 8.82 (s, 1H). | 3-methylaniline |

### Intermediate 255: 1-{3-[(5-bromo-4-chloropyrimidin-2-yl)amino]phenyl}ethanone

### Intermediate 256: 5-bromo-4-chloro-N-(3-methylphenyl)pyrimidin-2-amine

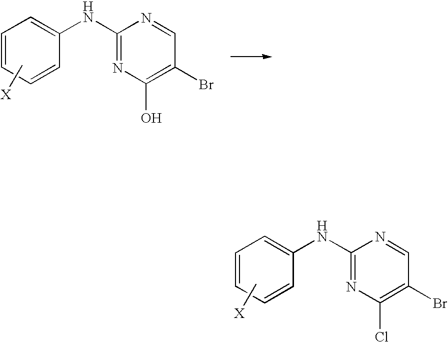

A solution of 5-bromo-2-[arylamino]pyrimidin-4-ol (1 eq) in POCl₃ (3-10 v/w) was heated to reflux for 1 h. It was cooled to RT and POCl₃ was removed in vacuo. To the residue obtained, was added crushed ice with stirring, and the pH was adjusted to 7-8 using 10% sodium bicarbonate solution. The solid obtained was filtered and washed with chilled water to yield the title compound. The compounds in the below table were prepared using this method and the specified starting material.

| Compound | Structure | Mass sperctrum and ¹H NMR | SM |
|---|---|---|---|
| Intermediate 255^{a)} | 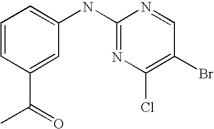 1-{3-[(5-bromo- 4- chloropyrimidin- 2- yl)amino]phenyl} ethanone | MS(ES): 326 (M) and 328 (M + 2) for C₁₂H₉BrClN₃O. 400 MHz, DMSO-d₆: δ 2.56 (s, 3H), 7.47 (t, J = 7.88 Hz, 1H), 7.62-7.62 (m, 1H), 7.89-7.90 (m, 1H), 8.26 (t, J = 1.88 Hz, 1H), 8.72 (s, 1H), 10.39 (s, 1H). | Intermediate 253 1-{3-[(5-bromo-4- hydroxypyrimidin- 2- yl)amino]phenyl} ethanone |
| Intermediate 256 | 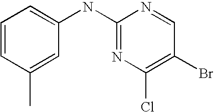 5-bromo-4- chloro-N-(3- methylphenyl) pyrimidin-2- amine | MS(ES): 298 (M) and 300 (M + 2) for C₁₁H₉BrClN₃. 300 MHz, DMSO-d₆: δ 2.27 (s, 3H), 6.83 (d, J = 7.47 Hz, 1H), 7.18 (t, J = 7.83 Hz, 1H), 7.44 (d, J = 4.71 Hz, 1H), 7.48 (s, 1H), 8.65 (s, 1H), 10.11 (s, 1H). | Intermediate 254 5-bromo-2-[(3- methylphenyl) amino] pyrimidin-4-ol |

^{a)}N,N-diethylaniline (0.2 eq) and MeCN (10 v/w) were also added

### General method for the synthesis of 5-bromo-N-(aryl)-4-[3-(trifluoromethyl)-1H-pyrazol-1-yl]pyrimidin-2-amine and 5-bromo-N-(aryl)-4-[5-methyl-3-(trifluoromethyl)-1H-pyrazol-1-yl]pyrimidin-2-amine

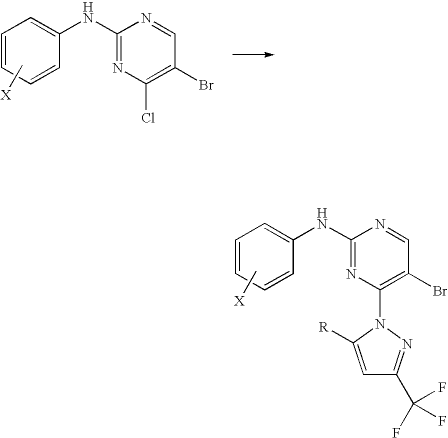

NaH (1.2 eq) was dissolved in 1 mL of NMP and stirred for about 5 min at 0° C. Then 3-(trifluoromethyl)-1H-pyrazole/5-methyl-3-(trifluoromethyl)-1H-pyrazole (1.1 eq) in NMP was added dropwise at 0° C. for about 10 min and stirring continued for about 20 min under N₂. Then 5-bromo-4-chloro-N-(aryl)pyrimidin-2-amine (1 eq) in NMP was added dropwise and the reaction was stirred overnight at room temperature. Water was added and the solid obtained was filtered, dried and purified by silica gel column chromatography using ethyl acetate/hexanes as eluent to yield the desired product. The compounds in the below table were prepared using this method and the specified starting material.

| Compound | Structure | Mass spectrum and ¹H NMR | SM |
|---|---|---|---|
| Intermediate 257 | 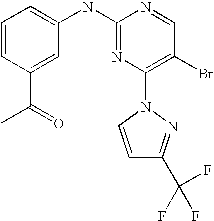 1-[3-({5-bromo-4- [3- (trifluoromethyl)- 1H-pyrazol-1- yl]pyrimidin-2- yl}amino)phenyl] ethanone | MS(ES): 426 (M) and 428 (M + 2) for C₁₆H₁₁BrF₃N₅O. 400 MHz, DMSO-d₆: δ 2.56 (s, 3H), 7.47 (t, J = 7.88 Hz, 1H), 7.62-7.65 (m, 1H), 7.89-7.92 (m, 1H), 8.26 (t, J = 1.88 Hz, 1H), 8.72 (s, 1H), 10.39 (s, 1H) | Intermediate 255 (1-{3-[(5- bromo-4- chloropyri- din-2- yl)amino]- phenyl}- ethanone) |
| Intermediate 258 | 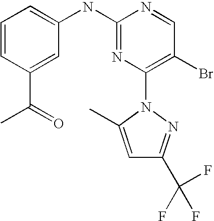 1-[3-({5-bromo-4- [5-methyl-3- (trifluoromethyl)- 1H-pyrazol-1- yl]pyrimidin-2- yl}amino)phenyl] ethanone | MS(ES): 440 (M) and 442 (M + 2) for C₁₇H₁₃BrF₃N₅O. 400 MHz, DMSO-d₆: δ 2.41 (s, 3H), 2.54 (s, 3H), 6.85 (s, 1H), 7.48 (t, J = 9.20 Hz, 1H), 7.65 (d, J = 7.00 Hz, 1H), 7.87 (d, J = 8.20 Hz, 1H), 8.29 (s, 1H), 8.96 (s, 1H), 10.45 (s, 1H). | Intermediate 255 (1-{3-[(5- bromo-4- chloro- pyrimidin-2- yl)amino] phenyl} ethanone) |
| Intermediate 259^{b)} | 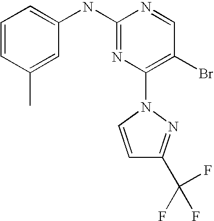 5-bromo-N-(3- methylphenyl)-4- [3- (trifluoromethyl)- 1H-pyrazol-1- yl]pyrimidin-2- amine | MS(ES): 398 (M) and 400 (M + 2) for C₁₅H₁₁BrF₃N₅. 300 MHz, DMSO-d₆: δ 2.28 (s, 3H), 6.84 (d, J = 7.59 Hz, 1H), 7.12 (d, J = 2.43 Hz, 1H), 7.19 (t, J = 7.89 Hz, 1H), 7.51 (d, J = 5.34 Hz, 1H), 7.58 (s, 1H), 8.61 (d, J = 0.96 Hz, 1H), 8.85 (d, J = 1.41 Hz, 1H), 10.15 (s, 1H). | Intermediate 256 5-bromo-4- chloro-N-(3- methylphenyl) pyrimidin-2- amine |
| Intermediate 260 | 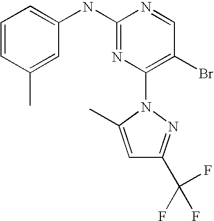 5-bromo-N-(3- methylphenyl)-4- [5-methyl-3- (trifluoromethyl)- 1H-pyrazol-1- yl]pyrimidin-2- amine | MS(ES): 412 (M) and 414 (M + 2) for C₁₆H₁₃BrF₃N₅ 400 MHz, DMSO-d₆: δ 2.27 (s, 3H), 2.39 (s, 3H), 6.85 (d, J = 9.96 Hz, 2H), 7.19 (t, J = 7.64 Hz, 1H), 7.46 (d, J = 7.80 Hz, 2H), 8.90 (d, J = 1.08 Hz, 1H), 10.19 (s, 1H). | Intermediate 256 5-bromo-4- chloro-N-(3- methylphenyl) pyrimidin-2- amine |

^{b)}DMF was used as solvent, 12 h

### Intermediate 261: 5-bromo-2-chloro-4-[5-methyl-3-(trifluoromethyl)-1H-pyrazol-1-yl]pyrimidine

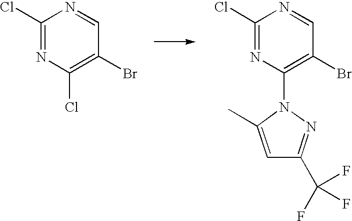

To a suspension of 5-bromo-2,4-dichloropyrimidine (44 mmol, 10 g) in acetonitrile (100 mL), was added K₂CO₃ (44 mmol, 6.1 g) and the reaction mixture was cooled to −5 to −10° C. 5-methyl-3-(trifluoromethyl)-1H-pyrazole (44 mmol, 6.6 g) was dissolved in 100 mL of acetonitrile and added dropwise. After the addition, the reaction mixture was slowly warmed to RT and stirred overnight. The reaction mixture was filtered through a celite bed and acetonitrile was removed in vacuo. The crude mass was purified by silica gel column chromatography (60-120 mesh; product eluted with 1% EtOAc/hexanes) to yield 5 g of the product.

| Compound | Structure | Mass spectrum and ¹H NMR | SM |
|---|---|---|---|
| Intermediate 261 | 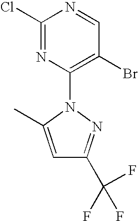 5-bromo-2-chloro- 4-[5-methyl-3- (trifluoromethyl)- 1H-pyrazol-1- yl]pyrimidine | MS(ES): 341 (M) and 343 (M + 2) for C₉H₅BrClF₃N₄. 300 MHz, CDCl₃: δ 2.52 (s, 3H), 6.53 (s, 1H), 8.94 (s, 1H). | 5-bromo-2,4- dichloro- pyrimidine and 5-methyl-3- (trifluoro- methyl)-1H- pyrazole |

### General method for the synthesis of 5-bromo-N-(aryl)-4-[3-(trifluoromethyl)-1H-pyrazol-1-yl]pyrimidin-2-amine and 5-bromo-N-(aryl)-4-[5-methyl-3-(trifluoromethyl)-1H-pyrazol-1-yl]pyrimidin-2-amine

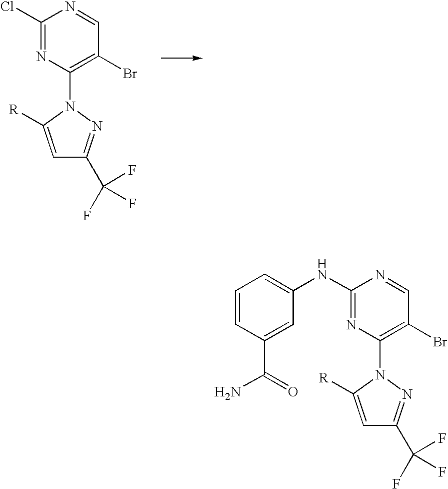

To a suspension of cesium carbonate (2 eq) in dry DMF (4 mL), were added 3-aminobenzamide (1.15 eq) and 5-bromo-2-chloro-4-[3-(trifluoromethyl)-1H-pyrazol-1-yl]pyrimidine (Intermediate 227, 1 eq) or 5-bromo-2-chloro-4-[5-methyl-3-(trifluoromethyl)-1H-pyrazol-1-yl]pyrimidine (Intermediate 261, 1 eq). The reaction vessel was sealed and heated to 100° C. for 5 h. The reaction mixture was diluted with EtOAc and filtered through a celite bed. The filtrate was washed with water and brine, dried over sodium sulfate and concentrated. The crude mass was purified by silica gel column chromatography (60-120 mesh). The column was eluted with 3% Methanol/Chloroform to yield the desired product. The compounds in the below table were prepared using this method and the specified starting material.

| Compound | Structure | Mass spectrum and ¹H NMR | SM |
|---|---|---|---|
| Intermediate 262 | 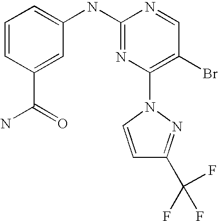 3-({5-bromo-4-[3- (trifluoromethyl)- 1H-pyrazol-1- yl]pyrimidin-2- yl}amino) benzamide | MS(ES): 427 (M) and 429 (M + 2) for C₁₅H₁₀BrF₃N₆O. 300 MHz, DMSO-d₆: δ 7.13 (d, J = 2.73 Hz, 1H), 7.38 (t, J = 7.83 Hz, 2H), 7.52 (d, J = 7.80 Hz, 1H), 7.76 (d, J = 9.90 Hz, 1H), 7.93 (s, 1), 8.27 (s, 1H), 8.68 (s, 1H), 8.88 (s, 1H), 10.35 (s, 1H). | Intermediate 227 5-bromo-2- chloro-4-[3- (trifluoro- methyl)-1H- pyrazol-1- yl]pyrimidine |
| Intermediate 263 | 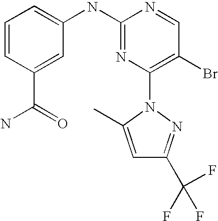 3-({5-bromo-4-[5- methyl-3- (trifluoromethyl)- 1H-pyrazol-1- yl]pyrimidin-2- yl}amino)benz- amide | MS(ES): 441 (M) and 443 (M + 2) for C₁₆H₁₂BrF₃N₆O. 300 MHz, DMSO-d₆: δ 2.48 (s, 3H), 6.63 (s, 1H), 7.34 (d, J = 7.68 Hz, 2H), 7.39 (d, J = 7.86 Hz, 1H), 7.51 (d, J = 7.80 Hz, 1H), 7.75 (d, J = 8.13 Hz, 1H), 7.89 (s, 1H), 8.14 (s, 1H), 8.92 (s, 1H), 10.34 (s, 1H). | Intermediate 261 5-bromo-2- chloro-4-[5- methyl-3- (trifluoro- methyl)-1H- pyrazol-1- yl]pyrimidine |

### Intermediate 264: 3-{[5-bromo-4-(methylsulfanyl)pyrimidin-2-yl]amino}benzonitrile

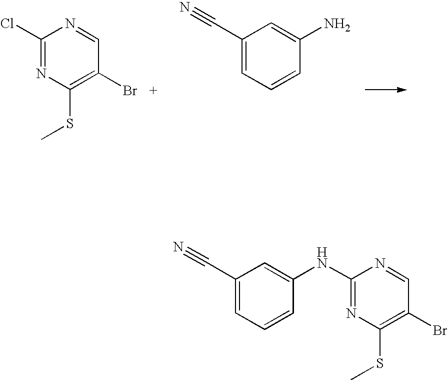

To 6 g of 5-bromo-2-chloro-4-(methylsulfanyl)pyrimidine (1.7 eq) suspended in n-BuOH (20 v/w), was added 3-aminobenzonitrile (1 eq) followed by 4M HCl in dioxane (0.25 eq) and refluxed overnight at 100° C. The reaction was cooled to room temperature, diluted with diethyl ether and the solid was filtered and dried to give Intermediate 264.

| Compound | Structure | Mass sperctrum and ¹H NMR | SM |
|---|---|---|---|
| Intermediate 264 | 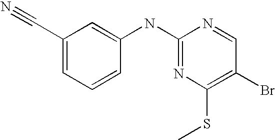 3-{[5-bromo-4- (methylsulfanyl) pyrimidin-2- yl]amino} benzonitrile | Taken to the next step to the basis of LCMS. MS(ES): 321 (M) and 323 (M + 2) for C₁₂H₉BrN₄S. (94% pure by LCMS) | 3- aminobenzo- nitrile and (5- bromo-2- chloro-4- (methylsul- fanyl)pyrimidine |

### Intermediate 265: 3-{[5-bromo-4-(methylsulfonyl)pyrimidin-2-yl]amino}benzonitrile

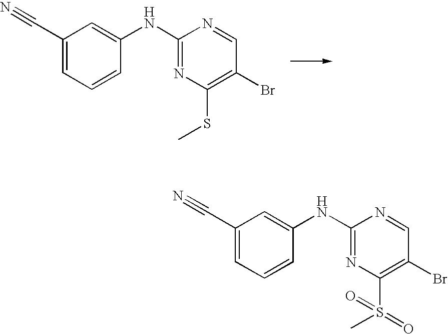

A suspension of 3-{[5-bromo-4-(methylsulfanyl)pyrimidin-2-yl]amino}benzonitrile (Intermediate 264, 1 eq) in dichloromethane (50 v/w) was cooled to 0° C. and 3-chloroperoxybenzoic acid (77%, 4.5 eq) was added portion wise and stirred for 4 h. It was further diluted with dichloromethane and washed with 10% aq. NaHCO₃ solution and water, dried over Na₂SO₄, filtered and concentrated to give Intermediate 265.

| Compound | Structure | Mass spectrum and ¹H NMR | SM |
|---|---|---|---|
| Intermediate 265 | 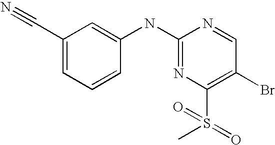 3-{[5-bromo-4- (methylsulfonyl) pyrimidin-2- yl]amino} benzonitrile | MS(ES): 353 (M) and 355 (M + 2) for C₁₂H₉BrN₄O₂S. 300 MHz, DMSO-d6: δ 3.47 (s, 3H), 7.47-7.57 (m, 2H), 7.90 (d, J = 8.13 Hz, 1H), 8.17 (s, 1H), 8.97 (s, 1H), 10.69 (s, 1H). | Intermediate 264 3-{[5-bromo-4- (methylsulfanyl) pyrimidin-2- yl]amino}benzo- nitrile |

### Intermediate 266: 3-({5-bromo-4-[3-(trifluoromethyl)-1H-pyrazol-1-yl]pyrimidin-2-yl}amino)benzonitrile

### Intermediate 267: 3-({5-bromo-4-[5-methyl-3-(trifluoromethyl)-1H-pyrazol-1-yl]pyrimidin-2-yl}amino)benzonitrile

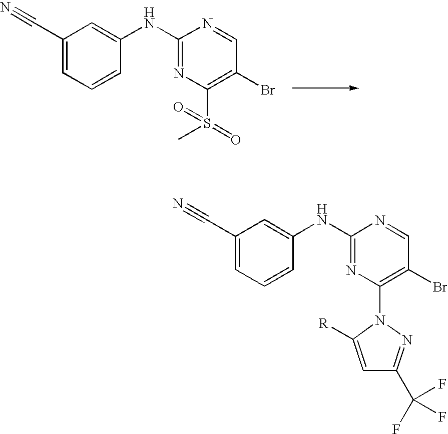

NaH (60% dispersion in mineral oil, 2 eq) was dissolved in 1 mL of DMF and stirred for about 5 min at 0° C. Then 3-(trifluoromethyl)-1H-pyrazole/5-methyl-3-(trifluoromethyl)-1H-pyrazole (2 eq) in DMF (2 mL) was added dropwise at 0° C. for about 10 min and stirring continued for about 20 min under N₂. Then 3-{[5-bromo-4-(methylsulfonyl)pyrimidin-2-yl]amino}benzonitrile (Intermediate 265, 1 eq) in DMF was added dropwise and the reaction was stirred for 2.5 h at room temperature. After completion of the reaction, water was added and the solid obtained was filtered off, dried and purified by silica gel column chromatography using ethyl acetate/hexanes as eluent to yield the title compound. The compounds in the below table were prepared using this method and the specified starting material.

| Compound | Structure | Mass spectrum and ¹H NMR | SM |
|---|---|---|---|
| Intermediate 266 | 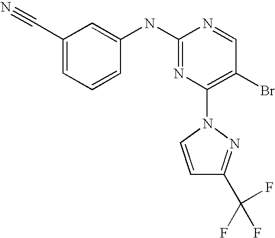 3-({5-bromo-4- [3- (trifluoromethyl)- 1H-pyrazol-1- yl]pyrimidin-2- yl}amino)ben- zonitrile | MS(ES): 409 (M) and 411 (M + 2) for C₁₅H₈BrF₃N₆. (83% pure by LCMS). 300 MHz, DMSO-d₆: δ 7.15 (d, J = 2.64 Hz,1H), 7.47 (d, J = 7.65 Hz, 1H), 7.53 (d, J = 7.95 Hz, 1H), 7.93 (br s, 1H), 8.20 (br s, 1H), 8.65 (br s, 1H), 8.96 (br s, 1H), 10.58 (br s, 1H). | Intermediate 265 3-{[5-bromo-4- (methylsulfonyl) pyrimidin-2- yl]amino}benzo- nitrile |
| Intermediate 267 | 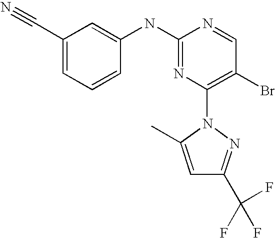 3-({5-bromo-4- [5-methyl-3- (trifluoromethyl)- 1H-pyrazol-1- yl]pyrimidin-2- yl}amino)ben- zonitrile | MS(ES): 423 (M) and 425 (M + 2) for C₁₆H₁₀BrF₃N₆. 300 MHz, DMSO-d₆: δ 2.39 (s, 3H), 6.86 (s, 1H), 7.47 (d, J = 7.68 Hz, 1H), 7.54 (t, J = 8.01 Hz, 1H), 7.89-7.92 (m, 1H), 8.14 (s, 1H), 9.00 (s, 1H), 10.60 (s, 1H). | Intermediate 265 3-{[5-bromo-4- (methylsulfonyl) pyrimidin-2- yl]amino}benzo- nitrile |

### Intermediate 268: (2E)-3-(3-{2-chloro-4-[3-(trifluoromethyl)-1H-pyrazol-1-yl]pyrimidin-5-yl}phenyl)prop-2-enoic acid

### Intermediate 269: (2E)-3-(3-{2-chloro-4-[5-methyl-3-(trifluoromethyl)-1H-pyrazol-1-yl]pyrimidin-5-yl}phenyl)prop-2-enoic acid

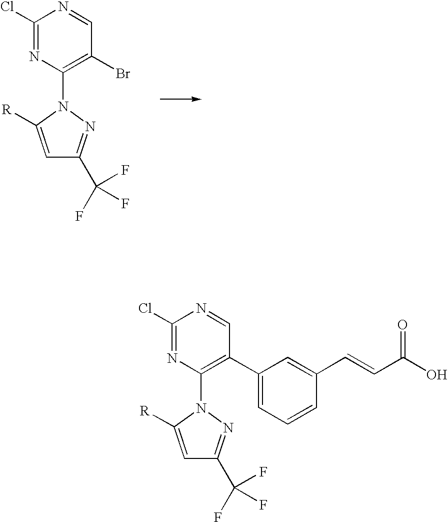

A suspension of 5-bromo-2-chloro-4-[3-(trifluoromethyl)-1H-pyrazol-1-yl]pyrimidine (Intermediate 227, 1 eq) or 5-bromo-2-chloro-4-[5-methyl-3-(trifluoromethyl)-1H-pyrazol-1-yl]pyrimidine (Intermediate 261, 1 eq), 3-(trans-2-carboxyvinyl)phenylboronic acid (1.1-1.2 eq), [1,1′-bis(diphenylphosphino)ferrocene]dichloropalladium(II) (10 mol %) and sodium carbonate (2 eq) in acetonitrile/water (4:1) was degassed and heated to 90° C. for 15-30 min under inert atmosphere. The solvent was removed in vacuo and the crude mixture taken in CHCl₃ was washed with water, brine, dried over Na₂SO₄, filtered and concentrated. The crude material was purified by silica gel column chromatography using 3-5% MeOH/CHCl₃ as an eluent to give the product. The compounds in the below table were prepared using this method and the specified starting material.

| Compound | Structure | Mass spectrum and ¹H NMR | SM |
|---|---|---|---|
| Intermediate 268 | 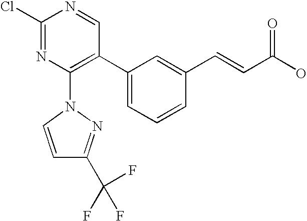 (2E)-3-(3-{2- chloro-4-[3- (trifluoromethyl)- 1H-pyrazol-1- yl]pyrimidin-5- yl}phenyl)prop-2- enoic acid | Taken to the next step on the basis of LCMS. MS(ES): 395 (M + 1) for C₁₇H₁₀ClF₃N₄O_{2.} | Intermediate 227 5-bromo-2- chloro-4-[3- (trifluoro- methyl)-1H- pyrazol-1- yl]pyrimidine |
| Intermediate 269 | 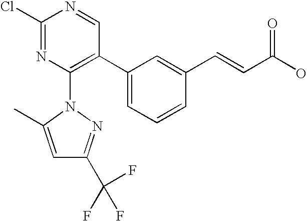 (2E)-3-(3-{2- chloro-4-[5- methyl-3- (trifluoromehtyl)- 1H-pyrazol-1- yl]pyrimidin-5- yl}phenyl)prop-2- enoic acid | Taken to the next step on the basis of LCMS. MS(ES): 409 (M + 1) for C₁₈H₁₂ClF₃N₄O_{2.} (75% pure by LCMS) | Intermediate 261 5-bromo-2- chloro-4-[5- methyl-3- (trifluoro- methyl)-1H- pyrazol-1- yl]pyrimidine |

### Intermediate 270: ethyl 5-{2-chloro-4-[3-(trifluoromethyl)-1H-pyrazol-1-yl]pyrimidin-5-yl]pyridine-3-carboxylate

### Intermediate 271: ethyl 5-{2-chloro-4-[5-methyl-3-(trifluoromethyl)-1H-pyrazol-1-yl]pyrimidin-5-yl]pyridine-3-carboxylate

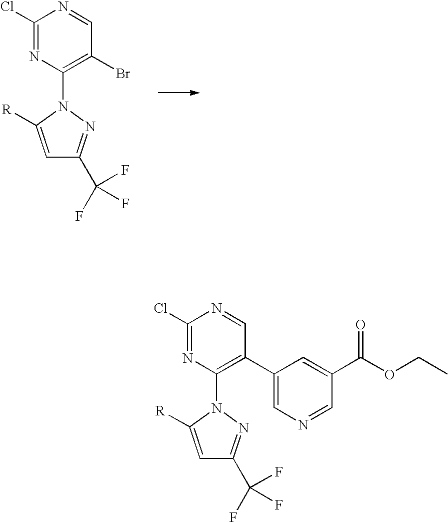

A solution of 5-bromo-2-chloro-4-[3-(trifluoromethyl)-1H-pyrazol-1-yl]pyrimidine (Intermediate 227, 1 eq) or 5-bromo-2-chloro-4-[5-methyl-3-(trifluoromethyl)-1H-pyrazol-1-yl]pyrimidine (Intermediate 261, 1 eq), ethyl 5-(4,4,5,5-tetramethyl-1,3,2-dioxaborolan-2-yl]pyridine-3-carboxylate (1 eq), [1,1′-bis(diphenylphosphino)ferrocene]dichloropalladium(II) (20 mol %) and sodium carbonate (1 eq) in acetonitrile (10 mL)/water (10 mL) was degassed and heated to 90° C. for 20′ under nitrogen. The reaction mixture was concentrated in vacuo. The residue obtained was taken in ethyl acetate, washed with water and brine, dried over sodium sulfate and concentrated. The crude mass was purified by silica gel column chromatography (60-120 mesh) using 35% ethyl acetate/hexanes as an eluent. The compounds in the below table were prepared using this method and the specified starting material.

| Compound | Structure | Mass spectrum and ¹H NMR | SM |
|---|---|---|---|
| Intermediate 270 | 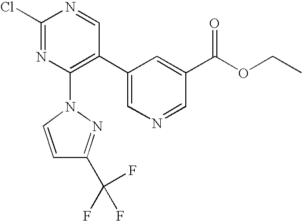 ethyl 5-{2-chloro- 4-[3- (trifluoromethyl)- 1H-pyrazol-1- yl]pyrimidin-5- yl}pyridine-3- carboxylate | MS(ES): 398 (M + 1) for C₁₆H₁₁ClF₃N₅O₂. 400 MHz, DMSO-d₆: δ 1.31 (t, J = 7.04 Hz, 3H), 4.35 (q, J = 7.08 Hz, 2H), 7.08 (d, J = 2.68 Hz, 1H), 8.26 (t, J = 2.08 Hz, 1H), 8.78 (m, 2H), 9.06 (s, 1H), 9.12 (d, J = 1.96 Hz, 1H). | Intermediate 227 5-bromo-2- chloro-4-[3- (trifluoro- methyl)-1H- pyrazol-1- yl]pyrimidine |
| Intermediate 271 | 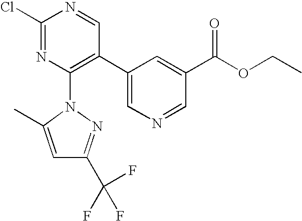 ethyl 5-{2-chloro- 4-[5-methyl-3- (trifluoromethyl)- 1H-pyrazol-1- yl]pyrazol-1- yl]pyrimidin-5- yl}pyridine-3- carboxylate | MS(ES): 412 (M + 1) for C₁₇H₁₃ClF₃N₅O₂. 400 MHz, DMSO-d₆: δ 1.31 (t, J = 7.08 Hz, 3H), 2.51 (s, 3H), 4.34 (q, J = 7.04 Hz, 2H), 6.84 (s, 1H), 8.04 (s, 1H), 8.69 (d, J = 1.88 Hz, 1H), 9.08 (d, J = 1.60 Hz, 1H), 9.26 (s, 1H). | Intermediate 261 5-bromo-2- chloro-4-[5- methyl-3- (trifluoro- methyl)-1H- pyrazol-1- yl]pyrimidine |

### Intermediate 272: methyl 5-{2-chloro-4-[3-(trifluoromethyl)-1H-pyrazol-1-yl]pyrimidin-5-yl}-2-methoxypyridine-3-carboxylate

### Intermediate 273: methyl 5-{2-chloro-4-[5-methyl-3-(trifluoromethyl)-1H-pyrazol-1-yl]pyrimidin-5-yl}-2-methoxypyridine-3-carboxylate

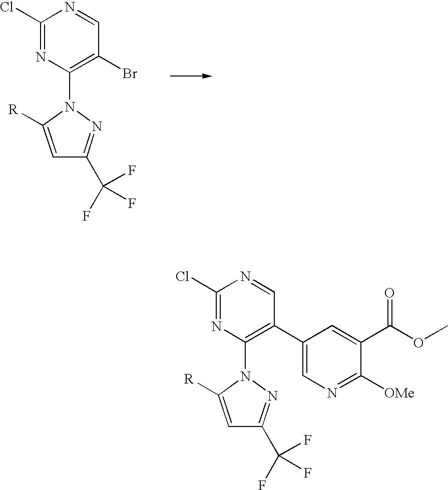

A solution of 5-bromo-2-chloro-4-[3-(trifluoromethyl)-1H-pyrazol-1-yl]pyrimidine (Intermediate 227, 1 eq) or 5-bromo-2-chloro-4-[5-methyl-3-(trifluoromethyl)-1H-pyrazol-1-yl]pyrimidine (Intermediate 261, 1 eq), methyl 2-methoxy-5-(4,4,5,5-tetramethyl-1,3,2-dioxaborolan-2-yl)pyridine-3-carboxylate (1 eq), [1,1′-bis(diphenylphosphino)ferrocene]dichloropalladium(II) (20 mol %) and sodium carbonate (1 eq) in acetonitrile (10 mL)/water (10 mL) was degassed and heated to 90° C. for 20 hours under nitrogen. The reaction mixture was concentrated in vacuo. The residue obtained was taken in ethyl acetate, washed with water and brine, dried over sodium sulfate and concentrated. The crude mass was purified by silica gel column chromatography (60-120 mesh) using 30% ethyl acetate/hexanes as an eluent to give the product. The compounds in the below table were prepared using this method and the specified starting material.

| Compound | Structure | Mass spectrum and ¹H NMR | SM |
|---|---|---|---|
| Intermediate 272 | 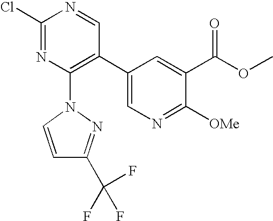 methyl 5-{2-chloro- 4-[3- (trifluoromethyl)- 1H-pyrazol-1- yl]pyrimidin-5-yl}- 2-methoxypyridine- 3-carboxylate | MS(ES): 414 (M + 1) for C₁₆H₁₁ClF₃N₅O₃. 400 MHz, DMSO-d₆: δ 3.78 (s, 3H), 3.96 (s, 3H), 7.08 (d, J = 2.72 Hz, 1H), 8.05 (d, J = 2.44 Hz, 1H), 8.36 (d, J = 2.48 Hz, 1H), 8.71 (t, J = 0.88 Hz, 1H), 9.04 (s, 1H). | Intermediate 227 |
| 5-bromo-2- chloro-4-[3- (trifluoro- methyl)-1H- pyrazol-1- yl]pyrimidine | | | |
| Intermediate 273 | 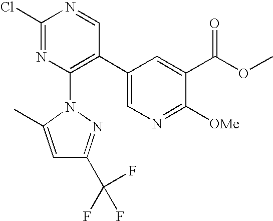 methyl 5-{2-chloro- 4-[5-methyl-3- (trifluoromethyl)- 1H-pyrazol-1- yl]pyrimidin-5-yl}- 2-methoxypyridine- 3-carboxylate | MS(ES): 428 (M + 1) for C₁₇H₁₃ClF₃N₅O₃. 300 MHz, DMSO-d₆: δ 2.40 (s, 3H), 3.80 (s, 3H), 3.93 (s, 3H), 6.82 (s, 1H), 7.75 (s, 1H), 8.29 (d, J = 2.46 Hz, 1H), 9.21 (s,1H). | Intermediate 261 5-bromo-2- chloro-4-[5- methyl-3- (trifluoro- methyl)-1H- pyrazol-1- yl]pyrimidine |

### Intermediate 274: 5-bromo-2-[(3-chlorophenyl)amino]pyrimidin-4-ol

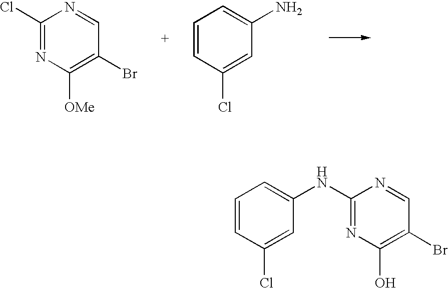

To a stirred solution of 5-bromo-2-chloro-4-methoxypyrimidine (53.8 mmol, 12 g) in n-BuOH (60 mL) was added 3-chloroaniline (59.1 mmol, 7.47 g, 6.2 mL) and 4 N HCl in dioxane (36 mL). The reaction mixture was heated at 80° C. for 20 h. It was then cooled to room temperature, acetonitrile (120 mL) was added and the resulting solid was filtered to yield 6 g of 5-bromo-2-[(3-chlorophenyl)amino]pyrimidin-4-ol as white solid.

| Compound | Structure | Mass spectrum and ¹H NMR | SM |
|---|---|---|---|
| Intermediate 274 | 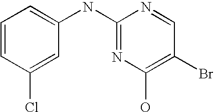 5-bromo-2-[(3- chlorophenyl) amino] pyrimidin-4- ol | MS (ES): 300 (M) and 302 (M + 2) for C₁₀H₇BrClN₃O. 400 MHz, DMSO-d₆: δ 7.09 (d, J = 7.12 Hz, 1H), 7.31-7.34 (m, 1H), 7.38 (s, 1H), 7.83 (s, 1H), 8.11 (s, 1H), 9.10 (sbr s, 1H), 11.52 (br s, 1H). | 5-bromo-2-chloro- 4- methoxypyrimidine and 3-chloroaniline |

### Intermediate 275: 5-bromo-4-chloro-N-(3-chlorophenyl)pyrimidin-2-amine

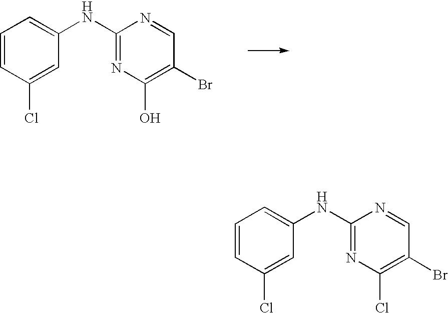

To 6 g of 5-bromo-2-[(3-chlorophenyl)amino]pyrimidin-4-ol (Intermediate 274, 20 mmol), was added 30 mL of POCl₃ (323 mmol, 49.5 g) and the mixture heated to reflux for 1 h. It was cooled to room temperature and POCl₃ was removed in vacuo. It was then diluted with water then extracted with ethyl acetate, organic layer washed successively with 10% sodium bicarbonate solution and water, dried over Na₂SO₄, filtered and concentrated to yield 3.0 g of 5-bromo-4-chloro-N-(3-chlorophenyl)pyrimidin-2-amine.

| Compound | Structure | Mass spectrum and ¹H NMR | SM |
|---|---|---|---|
| Intermediate 275 | 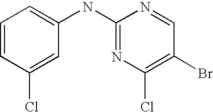 5-bromo-4-chloro-N-(3- chlorophenyl)pyrimidin-2-amine | MS (ES): 318 (M) and 320 (M + 2) for C₁₀H₆BrCl₂N₃. 400 MHz, DMSO-d₆: δ 7.05-7.07 (m, 1H), 7.33 (t, J = 8.16 Hz, 1H), 7.56-7.59 (m, 1H), 7.85 (t, J = 2.04 Hz, 1H), 8.74 (s,1H), 10.40 (s, 1H). | Intermediate 274 5-bromo-2-[(3- chlorophenyl)- amino]- pyrimidin-4-ol |

### Intermediate 276: 5-bromo-N-(3-chlorophenyl)-4-[3-(trifluoromethyl)-1H-pyrazol-1-yl]pyrimidin-2-amine

### Intermediate 277: 5-bromo-N-(3-chlorophenyl)-4-[5-methyl-3-(trifluoromethyl)-1H-pyrazol-1-yl]pyrimidin-2-amine

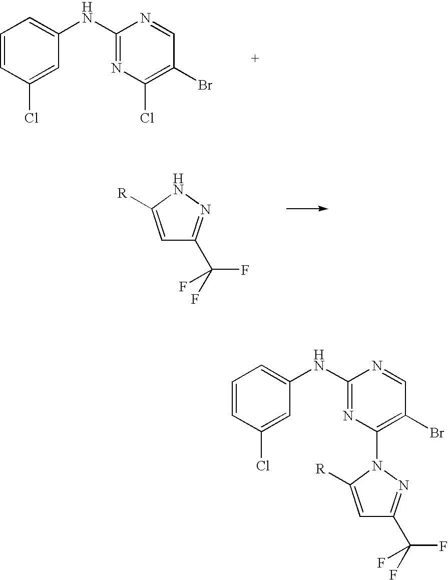

A solution of 3-(trifluoromethyl)-1H-pyrazole or 5-methyl-3-(trifluoromethyl)-1H-pyrazole (1.2 eq) in DMF (4.0 mL) was added slowly to a suspension of sodium hydride (1.2 eq) in DMF (4.0 mL) at 0° C. The reaction mixture was stirred for 1 h at room temperature. A solution of 5-bromo-4-chloro-N-(3-chlorophenyl)pyrimidin-2-amine (Intermediate 275, 1 eq) in DMF (4.0 mL) was added slowly to the reaction mixture at 0° C. and allowed to warm to ambient temperature over 2-3 h. The mixture was quenched with ice cold water at 0-5° C., pH adjusted to 2 with 1.5 N HCl and then extracted with ethyl acetate. The organic layer was washed with water, dried over Na₂SO₄, filtered and concentrated. The crude mass was purified by silica gel column chromatography (product eluted with 10-15% ethyl acetate/hexanes) to yield the desired product. The compounds in the below table were prepared using this method and the specified starting materials.

| | | Mass spectrum and ¹H | |
|---|---|---|---|
| Compound | Structure | NMR | SM |
| Intermediate 276 | 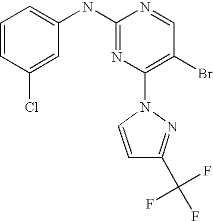 5-bromo-N-(3-chlorophenyl)-4-[3- (trifluoromethyl)-1H-pyrazol-1- yl]pyrimidin-2-amine | MS (ES): 418 (M) and 420 (M + 2) for C₁₄H₈BrF₃N₅. 400 MHz, DMSO-d₆: δ 7.06- 7.09 (m, 1H), 7.15 (d, J = 2.72 Hz, 1H), 7.35 (t, J = 8.12 Hz, 1H), 7.61-7.64 (m, 1H), 7.91 (t, J = 2.00 Hz, 1H), 8.64 (t, J = 1.68 Hz, 1H), 8.94 (s, 1H), 10.44 (s, 1H). | Intermediate 275 5-bromo-4- chloro-N-(3- chlorophenyl)- pyrimidin- 2-amine and 3- (trifluoro- methyl)-1H- pyrazole |
| Intermediate 277 | 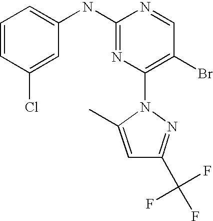 5-bromo-N-(3-chlorophenyl)-4-[5- methyl-3-(trifluoromethyl)-1H- pyrazol-1-yl]pyrimidin-2-amine | MS (ES): 432 (M) and 434 (M + 2) for C₁₅H₁₀BrClF₃N₅. 300 MHz, DMSO-d₆: δ 2.40 (s, 3H), 6.85 (s, 1H), 7.07 (d, J = 7.98 Hz, 1H), 7.34 (t, J = 8.07 Hz, 1H), 7.56 (d, J = 9.33 Hz, 1H), 7.84 (s, 1H), 8.98 (s, 1H), 10.46 (s, 1H). | 5-bromo-4- chloro-N-(3- chlorophenyl)- pyrimidin- 2-amine and 5-methyl-3- (trifluoro- methyl)-1H- pyrazole |

### Intermediate 278: 5-bromo-N-(3-fluoro-5-methylphenyl)-4-methoxypyrimidin-2-amine

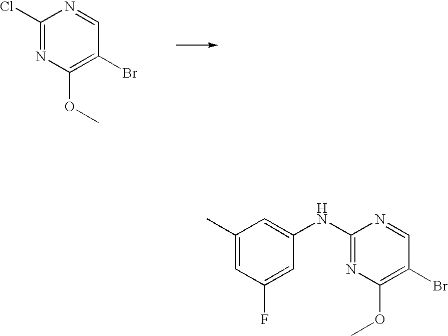

To a stirred solution of 5-bromo-2-chloro-4-methoxypyrimidine (1 eq) and 3-fluoro-5-methylaniline (1.01 eq) in n-BuOH (5 v/w based on the former) was added dioxane-HCl (3 v/w based on the former) and heated to 80° C. in a sealed tube for 3 h. The reaction mixture was cooled to room temperature and BuOH was concentrated under vacuum. To the residue obtained, was added MeCN and stirred at 10-15° C. for 30 minutes. The resulting solid was filtered and washed with MeCN to yield Intermediate 278.

| Compound | Structure | Mass spectrum and ¹H NMR | SM |
|---|---|---|---|
| Intermediate 278 | 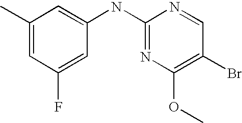 5-bromo-N-(3-fluoro-5- methylphenyl)-4-methoxypyrimidin- 2-amine | MS(ES): 312 (M) and 314 (M + 2) for C₁₂H₁₁BrFN₃O. (86% pure by LCMS) | 3-fluoro-5- methylaniline |

### Intermediate 279: 5-bromo-2-[(3-fluoro-5-methylphenyl)amino]pyrimidin-4-ol

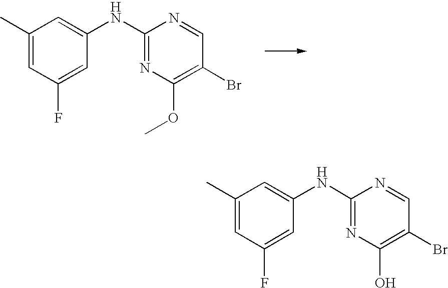

A mixture of 5-bromo-N-(3-fluoro-5-methylphenyl)-4-methoxypyrimidin-2-amine Intermediate 278 (1 eq) and aq. sodium thiomethoxide (21% aq. soln (w/v), 5 v/w based on Intermediate 278) and DMF (10 v/w) was heated to 80° C. for 2 h. The reaction mass was cooled to room temperature and quenched with ice-cold water and the pH adjusted to 2 with 1.5 N HCl solution. The precipitated solid was filtered and washed with water to yield Intermediate 279.

| Compound | Structure | Mass spectrum and ¹H NMR | SM |
|---|---|---|---|
| Intermediate 279 | 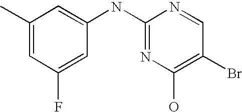 5-bromo-2-[(3-fluoro-5- methylphenyl)amino]- pyrimidin-4-ol | MS(ES): 298 (M) and 300 (M + 2) for C₁₁H₉BrFN₃O. 300 MHz, DMSO-d₆: δ 2.27 (s, 3H), 6.70 (d, J = 9.75 Hz, 1H), 7.03 (s, 1H), 7.45 (d, J = 11.58 Hz, 1H), 8.14 (s, 1H), 9.05 (br s, 1H), 11.45 (br s, 1H). | Intermediate 278 5-bromo-N-(3- fluoro-5- methylphenyl)- 4-methoxypyri- midin-2-amine |

### General procedure for the synthesis of 5-bromo-2-[aryl)amino]pyrimidin-4-ol

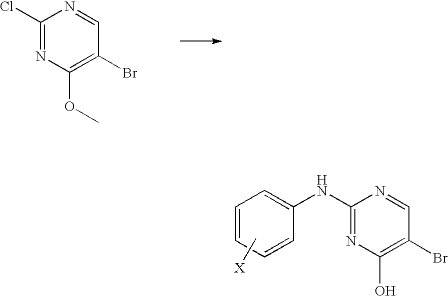

To a stirred solution of 5-bromo-2-chloro-4-methoxypyrimidine (1 eq) and arylamine (1.02 eq) in n-BuOH (12 v/w based on the former) was added 4M HCl in dioxane (4 v/w based on the former) and heated to 110° C. in a sealed tube for 18-36 h. The reaction mixture was cooled to room temperature and BuOH was concentrated under vacuum. To the residue obtained, was added water (50 mL) and 10% NaHCO₃ solution. The resulting solid was filtered and further purified by column chromatography to yield the product. The compounds in the below table were prepared using this procedure and the specified starting material.

| Compound | Structure | Mass spectrum and ¹H NMR | SM |
|---|---|---|---|
| Intermediate 280 | 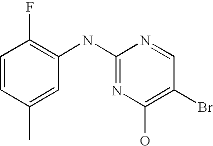 5-bromo-2-[(2-fluoro-5- methylphenyl)amino]- pyrimidin-4-ol | Taken to the next step based on LCMS without further purification. MS(ES): 298 (M) and 300 (M + 2) for C₁₁H₉BrFN₃O. | 2-fluoro-5- methylaniline |
| Intermediate 281 | 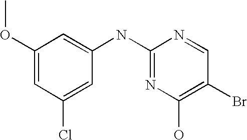 5-bromo-2-[(3-chloro-5- methoxyphenyl)amino]- pyrimidin-4-ol | MS(ES): 330 (M) and 332 (M + 2) for C₁₁H₉BrClN₃O₂. 400 MHz, DMSO-d₆: δ 3.76 (s, 3H), 6.72 (t, J = 2.04 Hz, 1H), 7.11 (t, J = 1.88 Hz, 1H), 7.35 (t, J = 1.76 Hz, 1H), 8.12 (s, 1H), 9.45 (s, 1H), 11.53 (s, 1H). | 3-chloro-5- methoxyaniline |
| Intermediate 282 | 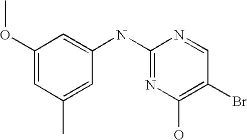 5-bromo-2-[(3-methoxy-5- methylphenyl)amino]- pyrimidin-4-ol | MS(ES): 310 (M) and 312 (M + 2) for C₁₂H₁₂BrN₃O₂. 400 MHz, DMSO-d₆: δ 2.26 (s, 3H), 3.72 (s, 3H), 6.47 (s, 1H), 6.89 (s, 1H), 7.12 (s, 1H), 7.91 (s, 1H), 9.56 (s, 1H), 11.53 (s, 1H). | 3-methoxy-5- methylaniline |

### General procedure for the synthesis of 5-bromo-4-chloro-N-(aryl)pyrimidin-2-amine

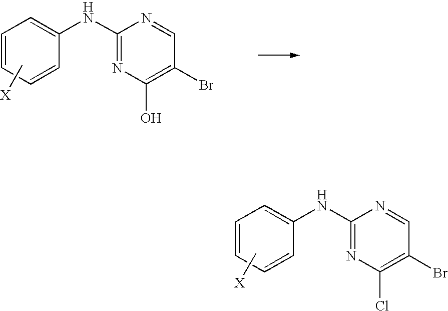

A solution of 5-bromo-2-[arylamino]pyrimidin-4-ol (1 eq) in POCl₃ (5 eq) was heated to reflux for 1 h. It was cooled to RT and POCl₃ was removed in vacuo. To the residue obtained, was added crushed ice with stirring, and the pH was adjusted to 7-8 using 10% NaHCO₃ solution. The solid obtained was filtered and washed with chilled water to yield the title compound. The compounds in the below table were prepared using this procedure and the specified starting material.

| Compound | Structure | Mass spectrum and ¹H NMR | SM |
|---|---|---|---|
| Intermediate 283 | 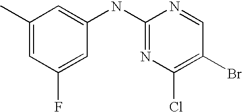 5-bromo-4-chloro-N-(3- fluoro-5-methylphenyl)- pyrimidin-2-amine | MS(ES): 316 (M) and 318 (M + 2) for C₁₁H₈BrClFN₃. 300 MHz, DMSO-d₆: δ 2.27 (s, 3H), 6.67 (d, J = 9.39 Hz, 1H), 7.21 (s, 1H), 7.48 (d, J = 11.70 Hz, 1H), 8.72 (s, 1H), 10.33 (s, 1H). | Intermediate 279 5-bromo- 2-[(3-fluoro- 5-methyl- phenyl)amino]- pyrimidin-4- ol |
| Intermediate 284^{a)} | 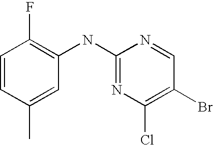 5-bromo-4-chloro-N-(2- fluoro-5-methylphenyl)- pyrimidin-2-amine | MS(ES): 316 (M) and 318 (M + 2) for C₁₁H₈BrClFN₃. 400 MHz, DMSO-d₆: δ 2.27 (s, 3H), 7.02 (d, J = 5.08 Hz, 1H), 7.12 (t, J = 8.48 Hz, 1H),7.30 (d, J = 7.76 Hz, 1H), 8.59 (s, 1H), 9.75 (s, 1H). | Intermediate 280 5-bromo- 2-[(2-fluoro- 5-mehtyl- phenyl)amino]- pyrimidin-4- ol |
| Intermediate 285^{a)} | 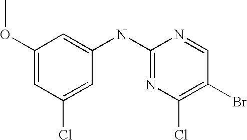 5-bromo-4-chloro-N-(3- chloro-5-methoxyphenyl)- pyrimidin-2-amine | MS(ES): 348 (M) and 350 (M + 2) for C₁₁H₈BrCl₂N₃O. 400 MHz, DMSO-d₆: δ 3.76 (s, 3H), 6.69 (t, J = 2.04 Hz, 1H), 7.31 (t, J = 2.08 Hz, 1H), 7.42 (t, J = 1.80 Hz, 1H), 8.75 (s, 1H), 10.37 (s, 1H). | Intermediate 281 5-bromo- 2-[(3-chloro- 5-methoxy- phenyl)amino]- pyrimidin-4- ol |
| Intermediate 286 | 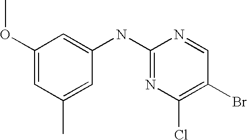 5-bromo-4-chloro-N-(3- methoxy-5-methylphenyl)- pyrimidin-2-amine | MS(ES): 328 (M) and 330 (M + 2) for C₁₂H₁₁BrClN₃O. 400 MHz, DMSO-d₆: δ 2.26 (s, 3H), 3.72 (s, 3H), 6.46 (s, 1H), 7.03 (s, 1H), 7.21 (s, 1H), 8.69 (s, 1H), 10.11 (s, 1H). | Intermediate 282 5-bromo-2- [(3-methoxy- 5- methylphenyl)- amino]pyri- midin-4-ol |

^{a)}N,N-diethylaniline (0.5 eq), Et₄N⁺Cl⁻ (0.5 eq) and MeCN (10 v/w) were also added

### General procedure for the synthesis of 5-bromo-N-(aryl)-4-[3-(trifluoromethyl)-1H-pyrazol-1-yl]pyrimidin-2-amine and 5-bromo-N-(aryl)-4-[5-methyl-3-(trifluoromethyl)-1H-pyrazol-1-yl]pyrimidin-2-amine

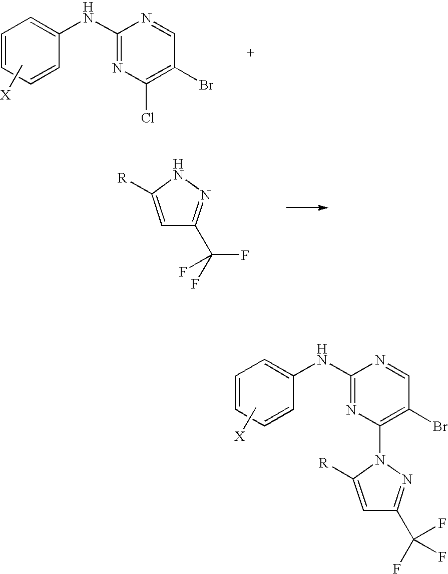

NaH (1.2 eq) was dissolved in 1 mL of DMF and stirred for about 5 min at 0° C. Then 3-(trifluoromethyl)-1H-pyrazole or 5-methyl-3-(trifluoromethyl)-1H-pyrazole (1.1 eq) in NMP was added drop-wise at 0° C. over about 10 min and stirring continued for about 20 min under N₂. Then 5-bromo-4-chloro-N-(aryl)pyrimidin-2-amine (1 eq) in DMF was added dropwise and the reaction was stirred overnight at RT. Water was added and the solid obtained was filtered, dried and purified by silica gel column chromatography using ethyl acetate/hexanes (2:8) as eluent to yield the product. The compounds in the below table were prepared using this procedure and the specified starting material.

| Compound | Structure | Mass spectrum and ¹H NMR | SM |
|---|---|---|---|
| Intermediate 287 | 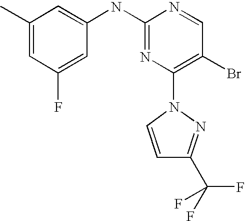 5-bromo-N-(3-fluoro-5- methylphenyl)-4-[3- (trifluoromethyl)-1H-pyrazol-1- yl]pyrimidin-2-amine | MS(ES): 416 (M) and 418 (M + 2) for C₁₅H₁₀BrF₄N₅. 300 MHz, DMSO-d₆: δ 2.28 (s, 3H), 6.68 (d, J = 9.54 Hz, 1H), 7.14 (d, J = 2.58 Hz, 1H), 7.30 (s, 1H), 7.49 (d, J = 11.73 Hz, 1H), 8.63 (d, J = 1.50 Hz, 1H), 8.91 (s, 1H), 10.37 (s, 1H). | Intermediate 283 5-bromo-4- chloro-N-(3- fluoro-5- methylphenyl)- pyrimidin-2- amine |
| Intermediate 288 | 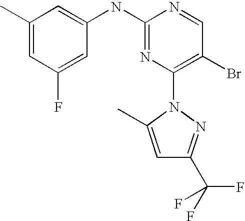 5-bromo-N-(3-fluoro-5- methylphenyl)-4-[5-methyl-3- (trifluoromethyl)-1H-pyrazol-1- yl]pyrimidin-2-amine | MS(ES): 430 (M) and 432 (M + 2) for C₁₆H₁₂BrF₄N₅. 400 MHz, DMSO-d₆: δ 2.27 (s, 3H), 2.39 (s, 3H), 6.69 (d, J = 9.08 Hz, 1H), 6.85 (s, 1H), 7.22 (s, 1H), 7.46 (d, J = 11.48 Hz, 1H), 8.96 (s, 1H). | Intermediate 283 5-bromo-4- chloro-N-(3- fluoro-5- methylphenyl)- pyrimidin-2- amine |
| Intermediate 289 | 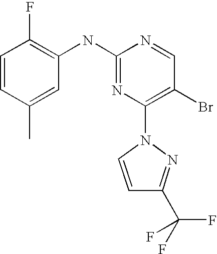 5-bromo-N-(2-fluoro-5- methylphenyl)-4-[3- (trifluoromethyl)-1H-pyrazol-1- yl]pyrimidin-2-amine | MS(ES): 416 (M) and 418 (M + 2) for C₁₅H₁₀BrF₄N₅. 400 MHz, DMSO-d₆: δ 2.28 (s, 3H), 7.01 (s, 1H), 7.09-7.10 (m, 2H), 7.42 (d, J = 6.84 Hz, 1H), 8.50 (s, 1H), 8.80 (s, 1H), 9.81 (s, 1H). | Intermediate 284 5-bromo-4- chloro-N-(2- fluoro-5- methylphenyl)- pyrimidin-2- amine |
| Intermediate 290 | 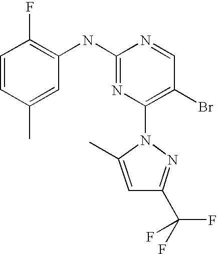 5-bromo-N-(2-fluoro-5- methylphenyl)-4-[5-methyl-3- (trifluoromethyl)-1H-pyrazol-1- yl]pyrimidin-2-amine | MS(ES): 430 (M) and 432 (M + 2) for C₁₆H₁₂BrF₄N₅. 400 MHz, DMSO-d₆: δ 2.27 (s, 3H), 2.33 (s, 3H), 6.81 (s, 1H), 7.02-7.03 (m, 1H), 7.15 (dd, J = 8.44, 10.58 Hz, 1H), 7.35 (t, J = 5.88 Hz, 1H), 8.85 (s, 1H), 9.85 (s, 1H). | Intermediate 284 5-bromo-4- chloro-N-(2- fluoro-5- methylphenyl)- pyrimidin-2- amine |
| Intermediate 291 | 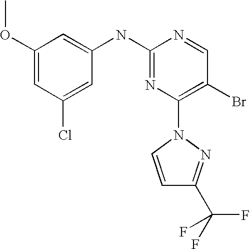 5-bromo-N-(3-chloro-5- methoxyphenyl)-4-[3- (trifluoromethyl)-1H-pyrazol-1- yl]pyrimidin-2-amine | MS(ES): 448 (M) and 450 (M + 2) for C₁₅H₁₀BrClF₃N₅O. 400 MHz, DMSO-d₆: δ 3.75 (s, 3H), 6.69 (s, 1H), 7.17 (d, J = 2.56 Hz, 1H), 7.42 (m, 2H), 8.65 (s, 1H), 8.95 (s, 1H), 10.42 (s, 1H). | Intermediate 295 5-bromo-4- chloro-N-(3- chloro-5- methoxy- phenyl)- pyrimidin- 2-amine |
| Intermediate 292 | 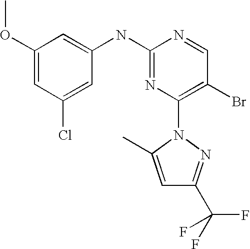 5-bromo-N-(3-chloro-5- methoxyphenyl)-4-[5-methyl-3- (trifluoromethyl)-1H-pyrazol-1- yl]pyrimidin-2-amine | MS(ES): 462 (M) and 464 (M + 2) for C₁₆H₁₂BrClF₃N₅O. 400 MHz, DMSO-d₆: δ 2.41 (s, 3H), 3.75 (s, 3H), 6.70 (s, 1H), 6.87 (s, 1H), 7.29 (d, J = 0.76 Hz, 1H), 7.42 (s, 1H), 9.00 (d, J = 1.04 Hz, 1H), 10.43 (s, 1H). | Intermediate 285 5-bromo-4- chloro-N-(3- chloro-5- methylphenyl)- pyrimidin-2- amine |
| Intermediate 293 | 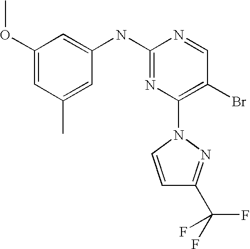 5-bromo-N-(3-methoxy-5- methylphenyl)-4-[3- (trifluoromethyl)-1H-pyrazol-1- yl]pyrimidin-2-amine | MS(ES): 428 (M) and 430 (M + 2) for C₁₆H₁₃BrF₃N₅O. 300 MHz, DMSO-d₆: δ 2.24 (s, 3H), 3.70 (s, 3H), 6.43 (s, 1H), 7.06 (s, 1H), 7.13 (d, J = 2.37 Hz, 1H), 7.26 (s, 1H), 8.62 (s, 1H), 8.86 (s, 1H), 10.15 (s, 1H). | Intermediate 286 5-bromo-4- chloro-N-(3- methoxy-5- methylphenyl)- pyrimidin-2- amine |
| Intermediate 294 | 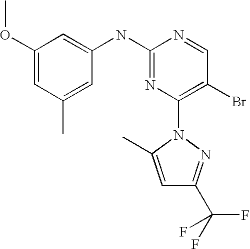 5-bromo-N-(3-methoxy-5- methylphenyl)-4-[5-methyl-3- (trifluoromethyl)-1H-pyrazol-1- yl]pyrimidin-2-amine | MS(ES): 442 (M) and 444 (M + 2) for C₁₇H₁₅BrF₃N₅O. 400 MHz, DMSO-d₆: δ 2.25 (s, 3H), 2.40 (s, 3H), 3.70 (s, 3H), 6.46 (s, 1H), 6.85 (s, 1H), 7.04 (s, 1H), 7.19 (s, 1H), 8.93 (s, 1H), 10.19 (s, 1H). | Intermediate 286 5-bromo-4- chloro-N-(3- methoxy-5- methylphenyl)- pyrimidin-2- amine |

### Intermediate 295: 5-bromo-2-[(1-methylpyrrolidin-3-yl)oxy]pyridine-3-carboxylic acid

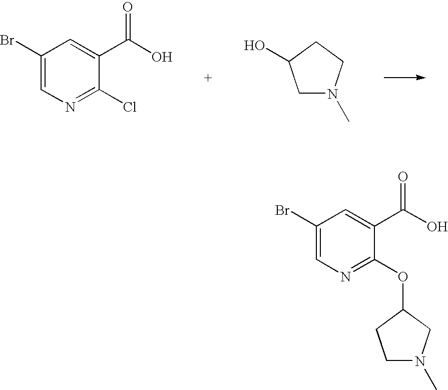

To 5-bromo-2-chloropyridine-3-carboxylic acid (4.21 mmol, 1 g) and 1-methylpyrrolidin-3-ol (17.5 mmol, 1.88 mL, 1.77 g) taken in tert-butanol (25 mL), was added sodium tert-butoxide (16.9 mmol, 1.64 g) and heated to 85° C. for 2 h. The solvent was removed in vacuo and the reaction mixture was diluted with water. The aqueous layer was washed with EtOAc, then carefully acidified with 1 N HCl (pH=5). Water was distilled off and the residue taken in a mixture of MeOH and EtOAc (1:1) was passed through was a celite bed and the resultant crude product (2 g) was used without further purification in the next step.

| | | Mass spectrum and ¹H | |
|---|---|---|---|
| Compound | Structure | NMR | SM |
| Intermediate 295 | 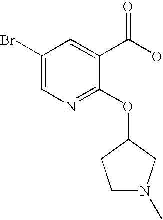 5-bromo-2-[(1- methylpyrrolidin-3- yl)oxy]pyridine-3- carboxylic acid | Taken to the next step based on LCMS without further purification. MS(ES): 301 (M) and 303 (M + 2) for C₁₁H₁₃BrN₂O₃. (92% pure by LCMS) | 1- methylpyrrolidin- 3-ol |

### Intermediate 296: methyl 5-bromo-2-[(1-methylpyrrolidin-3-yl)oxy]pyridine-3-carboxylate

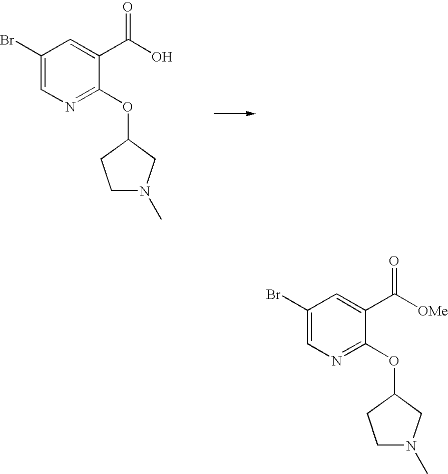

To a suspension of 5-bromo-2-[(1-methylpyrrolidin-3-yl)oxy]pyridine-3-carboxylic acid (Intermediate 295, 4.52 mmol, 1.36 g) in MeOH (13 ml) at 0° C., was slowly added thionyl chloride (8.4 mmol, 0.999 g). After the addition was complete, the reaction mixture was refluxed for 2 h. The solvent was concentrated in vacuo and the crude mixture was taken in EtOAc (30 mL), washed with aq. NaHCO₃ solution, water and brine, dried over Na₂SO₄, filtered and concentrated to obtain 1.2 g of the title compound.

| | | Mass spectrum and ¹H | |
|---|---|---|---|
| Compound | Structure | NMR | SM |
| Intermediate 296 | 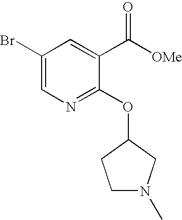 methyl 5-bromo-2-[(1- methylpyrrolidin-3- yl)oxy]pyridine-3- carboxylate | MS(ES): 315 (M) and 317 (M + 2) for C₁₂H₁₅BrN₂O₃. 400 MHz, DMSO-d₆: δ 1.18 (t, J = 7.12 Hz, 1H), 1.99 (s, 3H), 2.21-2.37 (m, 2H), 2.57-2.68 (m, 2H), 2.79 (dd, J = 6.24, 10.66 Hz, 1H), 3.82 (s, 3H), 5.33-5.38 (m, 1H), 8.49 (s, 1H), 8.49 (s, 1H). | Intermediate 295 5-bromo-2- [(1-methyl- pyrrolidin-3- yl)oxy]pyridine-3- carboxylic acid |

### Intermediate 297: {5-(methoxycarbonyl)-6-|(1-methylpyrrolidin-3-yl)oxy|pyridin-3-yl}boronic acid

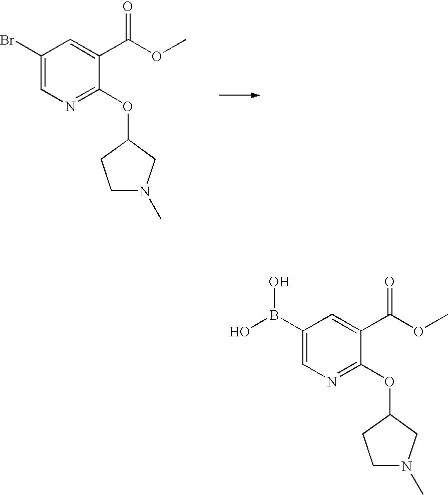

A suspension of methyl 5-bromo-2-[1-methylpyrrolidin-3-yl)oxy]pyridine-3-carboxylate (Intermediate 296, 1.5 mmol, 0.5 g), bis(pinacolato)diboron (3.1 mmol, 0.804 g), └1,1′-bis(diphenylphosphino)ferrocene┘dichloropalladium(II) (0.3 mmol, 0.219 g) and potassium acetate (4.6 mmol, 0.444 g) was taken in dioxane (5 mL) and it was degassed for 10 min. Then the reaction mixture was heated to 100° C. for 1 h. The reaction mixture was diluted with EtOAc, filtered through a celite bed, washed with water (5 mL) and brine (5 mL), dried over Na₂SO₄ and concentrated in vacuo to obtain the title compound as a crude mass (0.7 g) which was taken to the next step without further purification. HPLC-MS analysis indicates the presence of both the boronic acid and boronate pinacol ester.

| | | Mass spectrum and ¹H | |
|---|---|---|---|
| Compound | Structure | NMR | SM |
| Intermediate 297 | 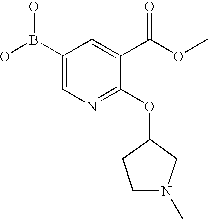 {5-(methoxycarbonyl)-6-[(1- methylpyrrolidin-3- yl)oxy]pyridin-3- yl}boronic acid | Taken to the next step as a mixture based on LCMS without further purification MS(ES): 281 (M + 1) for C₁₂H₁₇BN₂O₅ (40% as Boronic acid) and 363 (M + 1) for C₁₈H₂₇BN₂O₅ (20% as Boronic ester). | Intermediate 296 methyl 5- bromo-2-[(1- methyl pyrrolidin-3- yl)oxy] pyridine-3- carboxylate |

### Intermediate 298: 5-bromo-2-[1-(pyridin-4-yl)ethoxy]pyridine-3-carboxylic acid

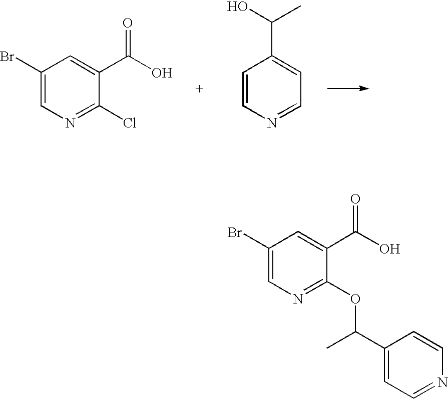

To 5-bromo-2-chloropyridine-3-carboxylic acid (4.2 mmol, 1 g) and 1-(pyridin-4-yl)ethanol (16.9 mmol, 2.08 g) taken in tert-butanol (20 mL), was added sodium tert-butoxide (16.9 mmol, 1.63 g) and the mixture heated to 85° C. for 1 h. The solvent was removed in vacuo and the reaction mixture was diluted with water. The aqueous layer was washed with EtOAc, then carefully acidified with 1 N HCl (pH=5). Water was distilled off and the residue taken in a mixture of MeOH and EtOAc (1:1) was passed through was a celite bed. The filtrate was concentrated in vacuo and the resultant crude product (2.2 g) was used in the next step without further purification.

| | | Mass spectrum and ¹H | |
|---|---|---|---|
| Compound | Structure | NMR | SM |
| Intermediate 298 | 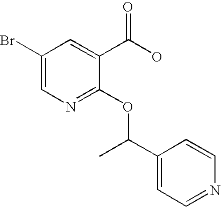 5-bromo-2-[1-(pyridin-4- yl)ethoxy]pyridine-3- carboxylic acid | MS(ES): 323 (M) and 325 (M + 2) for C₁₃H₁₁BrN₂O₃. The compound was taken to the next step on the basis of LCMS. | 1-(pyridin- 4-yl)ethanol |

### Intermediate 299: methyl5-bromo-2-[1-(pyridin-4-yl)ethoxy]pyridine-3-carboxylate

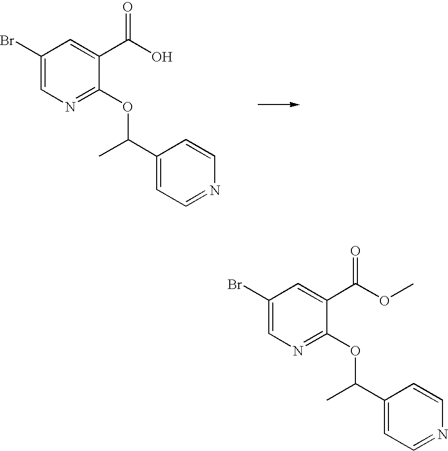

A solution of 5-bromo-2-[1-(pyridin-4-yl)ethoxy]pyridine-3-carboxylic acid (Intermediate 298, 6.81 mmol, 2.2 g) taken in MeOH (20 mL) was cooled to 0° C. Thionyl chloride (0.99 mL, 13.62 mmol, 2 eq) was added slowly to the reaction mixture. After the addition was complete, the reaction mixture was refluxed for 2 h. The solvent was removed in vacuo and the crude mixture was taken in EtOAc (30 mL), washed with aq. NaHCO₃ solution, water, brine, dried over Na₂SO₄, filtered and concentrated. The crude material was purified by silica gel column chromatography using 20% ethyl acetate/hexane as the eluent to yield 1 g of the title compound.

| | | Mass spectrum and ¹H | |
|---|---|---|---|
| Compound | Structure | NMR | SM |
| Intermediate 299 | 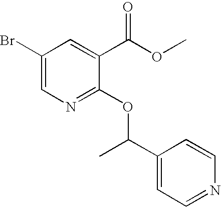 methyl 5-bromo-2-[1-(pyridin-4- yl)ethoxy]pyridine-3-carboxylate | MS(ES): 337 (M) and 339 (M + 2) for C₁₄H₁₃BrN₂O₃ 400 MHz, DMSO-d₆: δ 1.56 (d, J = 6.52 Hz, 3H),3.88 (s, 3H), 6.23 (q, J = 6.52 Hz, 1H), 7.45 (d, J = 6.00 Hz, 2H), 8.30 (d, J = 2.56 Hz, 1H), 8.44 (d, J = 2.56 Hz, 1H), 8.54 (dd, J = 1.52, 4.52 Hz, 2H). | Intermediate 298 5-bromo-2- [1-(pyridin- 4-yl)ethoxy] pyridine-3- carboxylic acid |

### Intermediate 300: methyl2-[1-(pyridin-4-yl)ethoxy]-5-(4,4,5,5-tetramethyl-1,3,2-dioxaborolan-2-yl)pyridine-3-carboxylate

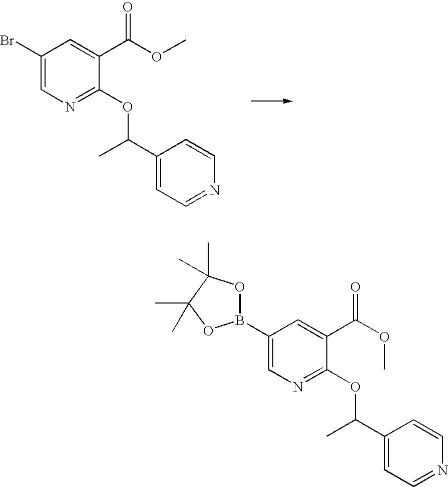

A suspension of methyl 5 -bromo-2-[1-(pyridin-4-yl)ethoxy]pyridine-3 -carboxylate (Intermediate 299, 1.3 mmol, 0.440 g), bis(pinacolato)diboron (2.61 mmol, 0.663 g), [1,1′-bis(diphenylphosphino)ferrocene]dichloropalladium(II) complex with CH₂Cl₂ (0.261 mmol, 0.213 g) and potassium acetate (3.91 mmol, 0.384 g) was taken in dioxane (10 mL) and it was degassed for 10 min. Then the reaction mixture was heated to 100° C. for 90 min. The reaction mixture was cooled to room temperature, diluted with ethyl acetate, filtered through a celite bed and concentrated in vacuo to yield the product as a crude mass (0.5 g) which was taken to the next step without further purification LCMS analysis indicated the presence of a mixture of Boronic acid (50%) and boronate (30%).

| | | Mass spectrum and ¹H | |
|---|---|---|---|
| Compound | Structure | NMR | SM |
| Intermediate 300 | 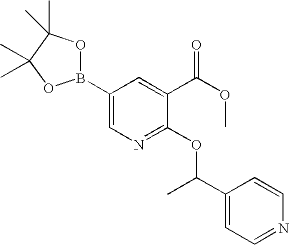 methyl 2-[1-(pyridin-4-yl)ethoxy]-5-(4,4,5,5- tetramethyl-1,3,2-dioxaborolan-2- yl)pyridine-3-carboxylate | Taken to the next step as a mixture based on LCMS without further purification MS(ES): 303 (M + 1) for C₁₄H₁₅BN₂O₅ (50% as Boronic acid) and 385 (M + 1) for C₂₀H₂₅BN₂O₅ (30% as Boronic ester). | Intermediate 299 methyl 5- bromo-2-[1- (pyridin-4- yl)ethoxy]- pyridine-3- carboxylate |

### Intermediate 301: 5-bromo-2-[1-(1H-imidazol-1-yl)ethoxy]pyridine-3-carboxylic acid

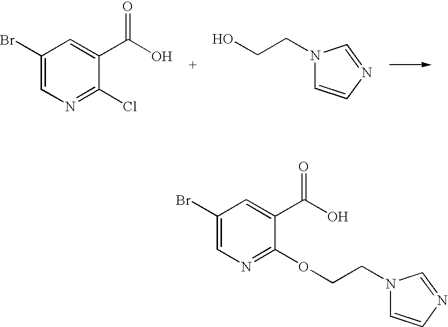

To 5-bromo-2-chloropyridine-3-carboxylic acid (4 2 mmol, 1 g) and 2-(1H-imidazol-1-yl)ethanol (12.6 mmol, 1.42 g) taken in tert-butanol (25 mL) was added sodium tert-butoxide (12.7 mmol, 1.231 g) and the reaction mixture heated to 90° C. for 2 h. The solvent was removed in vacuo and the reaction mixture was diluted with water. The aqueous layer was washed with EtOAc, then carefully acidified with 1 N HCl (pH=5). Water was distilled off and the residue taken in a mixture of MeOH and EtOAc (1:1) was passed through was a celite bed. The filtrate was concentrated in vacuo and the resultant crude product was taken to the next step without further purification (1.3 g).

| | | Mass spectrum and ¹H | |
|---|---|---|---|
| Compound | Structure | NMR | SM |
| Intermediate 301 | 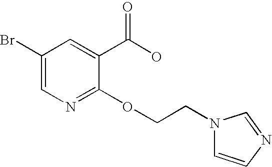 5-bromo-2-[2-(1H-imidazol-1- yl)ethoxy]pyridine-3-carboxylic acid | MS(ES): 312 (M) and 314 (M + 2) for C₁₁H₁₀BrN₃O₃. 400 MHz, DMSO-d₆: δ 4.32 (t, J = 4.52 Hz, 2H), 4.43 (t, J = 4.96 Hz, 2H), 6.88 (s, 1H), 7.15 (s, 1H), 7.60 (s, 1H), 7.89 (d, J = 2.56 Hz, 1H), 8.12 (d, J = 2.52 Hz, 1H), 10.42 (s, 1H). | 2-(1H- imidazol-1- yl)ethanol |

### Intermediate 302: methyl 5-bromo-2-[2-(1H-imidazol-1-yl)ethoxy]pyridine-3-carboxylate

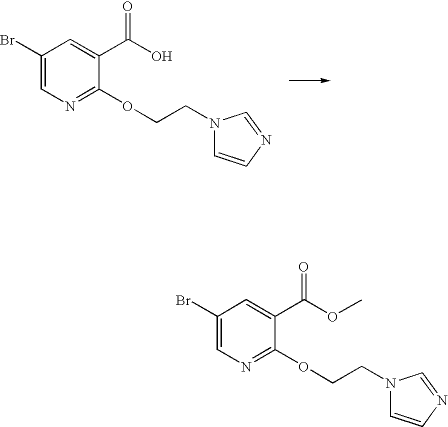

To a suspension of 5-bromo-2-[2-(1H-imidazol-1-yl)ethoxy]pyridine-3-carboxylic acid (Intermediate 301, 4.16 mmol, 1.3 g) in MeOH (50 mL) at 0° C., was slowly added thionylchloride (6.4 mmol, 0.74 g). After the addition was complete, the reaction mixture was refluxed at 85° C. for 2 h. The solvent was removed in vacuo and the crude mixture taken in EtOAc (30 mL), was washed with aq. NaHCO₃ solution, water and brine, dried over Na₂SO₄, filtered and concentrated to obtain 1.3 g of the title compound which was taken as such to the next step.

| | | Mass spectrum and ¹H | |
|---|---|---|---|
| Compound | Structure | NMR | SM |
| Intermediate 302 | 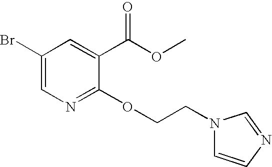 methyl 5-bromo-2-[2-(1H-imidazol-1- yl)ethoxy]pyridine-3-carboxylate | MS(ES): 326 (M) and 328 (M + 2) for C₁₂H₁₂BrN₃O₃. 400 MHz, DMSO-d₆: δ 3.84 (s, 3H), 4.38 (t, J = 4.60 Hz, 2H), 4.54 (t, J = 5.16 Hz, 2H), 6.88 (s, 1H), 7.28 (s, 1H), 7.99 (s, 1H), 8.29 (t, J = 2.56 Hz, 1H), 8.50 (d, J = 2.56 Hz, 1H). | Intermediate 301 5-bromo-2- [2-(1H- imidazol-1- yl)ethoxy]- pyridine-3- carboxylic acid |

### Intermediate 303: {6-[2-(1H-imidazol-1-yl)ethoxy]-5-(methoxycarbonyl)pyridin-3-yl}boronic acid

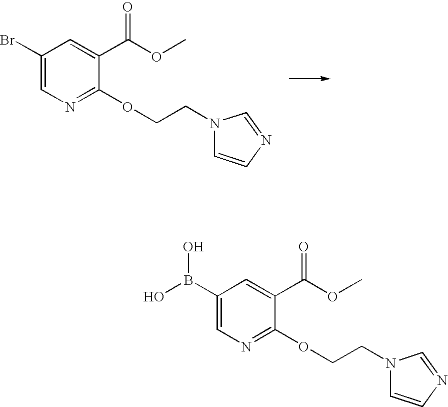

A suspension of Intermediate 302 (1.53 mmol, 0.5 g), bis(pinacolato)diboron (3.06 mmol, 0.778 g), [1,1′-bis(diphenylphosphino)ferrocene]dichloropalladium(II) (0.31 mmol, 0.224 g) and potassium acetate (4.6 mmol, 0.452 g) was taken in dioxane (50 mL) and it was degassed for 10 min. Then the reaction mixture was heated to 100° C. for 1 h. The reaction mixture was cooled to room temperature, diluted with ethyl acetate, filtered through a celite bed and concentrated in vacuo to obtain 0.7 g of the crude mass which was taken to the next step without further purification. LCMS analysis indicated the presence of a mixture of boronic acid (42%) and boronate (17%).

| | | Mass spectrum and ¹H | |
|---|---|---|---|
| Compound | Structure | NMR | SM |
| Intermediate 303 | 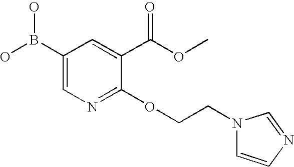 {6-[2-(1H-imidazol-1- yl)ethoxy]-5- (methoxycarbonyl)- pyrimidin-3-yl}boronic acid | Taken to the next step as a mixture based on LCMS without further purification MS(ES): 292 (M + 1) for C₁₂H₁₄BN₃O₃ (42% as boronic acid) and 374 (M + 1) for C₁₈H₂₄BN₃O₅ (17% as boronic ester). | Intermediate 302 methyl 5- bromo-2-[2- (1H- imidazol-1- yl)ethoxy]- pyridine-3- carboxylate |

### Intermediate 304: ethyl5-bromo-2-(1,3-dimethoxypropan-2-yloxy)nicotinate

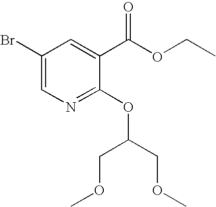

To a suspension of t-BuONa (4.36 g, 45 mmol) in THF (100 mL) was added a solution of 1,3-dimethoxypropan-2-ol (4.55 g, 37.8 mmol in 50 mL THF) at 0° C. over a period of 30 min. The reaction mixture was stirred at 10° C. for 1 h and then cooled to 0° C. To this reaction mixture was added a solution of ethyl 5-bromo-2-chloronicotinate (10.0 g, 37 8 mmol in 100 mL THF) over a period of 45 min. The reaction mixture was allowed to come to room temperature and stirred for 2 h. The reaction mixture was quenched with cold water (200 mL) and extracted with ethyl acetate (200 mL×3). The combined organic layer was washed with water (200 mL), brine solution (200 mL), dried over anhydrous Na₂SO₄ and evaporated under reduced pressure to get crude compound. The crude compound was purified by column chromatography (Silica gel, 100-200 mesh) using 6% ethyl acetate in pet ether as mobile phase to get 6.5 g of the title compound. The compounds in the below table were prepared using this method and the indicated starting material.

| Compound | Structure | Mass spectrum | SM |
|---|---|---|---|
| Intermediate 305 | 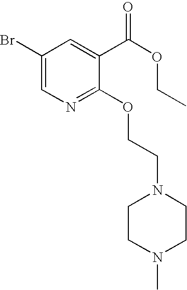 ethyl 5-bromo-2-(2-(4- methylpiperazin-1- yl)ethoxy)nicotinate | MS(ES): 372 (M + 1) for C₁₅H₂₂BrN₃O₃ | 2-(4- methylpiper azin-1- yl)ethanol |
| Intermediate 306 | 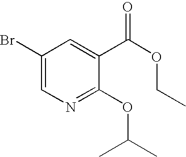 ethyl 5-bromo-2- isopropoxynicotinate | MS(ES): 288 (M + 1) for C₁₁H₁₄BrNO₃ | iospropanol |
| Intermediate 307 | 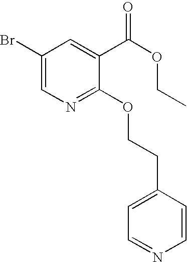 ethyl 5-bromo-2-(2- (pyridin-4- yl)ethoxy)nicotinate | MS(ES): 351 (M + 1) for C₁₅H₁₅BrN₂O₃ | 2-(pyridin- 4-yl)ethanol |
| Intermediate 308 | 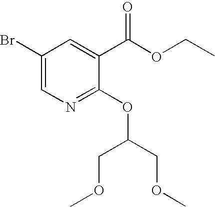 ethyl 5-bromo-2-(1,3- dimethoxypropan-2- yloxy)nicotinate | MS(ES): 349 (M + 1) for C₁₃H₁₈BrNO₅ | 1,3- dimethoxy- propan-2-ol |
| Intermediate 309 | 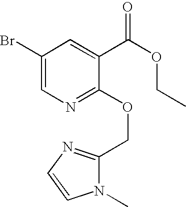 ethyl 5-bromo-2-((1- methyl-1H-imidazol-2- yl)methoxy)nicotinate | MS(ES): 340 (M + 1) for C₁₃H₁₄BrN₃O₃ | (1-methyl- 1H- imidazol-2- yl)methanol |

### Intermediate 310: 2-(2-acetamidoethoxy)-5-bromonicotinic acid

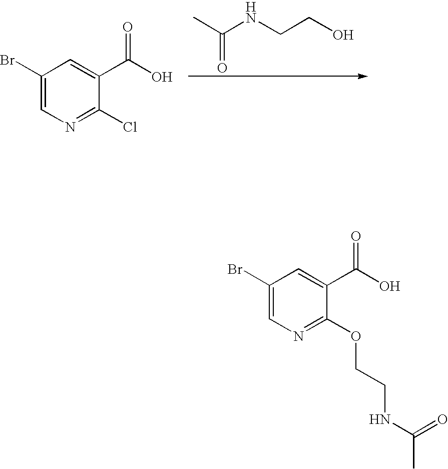

5-Bromo-2-chloronicotinic acid (1.25 g, 5.27 mmol) was suspended in tert-butanol (35.4 ml) and N-(2-hydroxyethyl)acetamide (1.95 ml, 21.08 mmol) was added. Potassium tert-butoxide (2.37 g, 21.08 mmol) was added and the reaction mixture was heated at 90° C. for 1 hour. Tert-butanol was removed in vacuo and the resulting material was diluted in ethyl acetate and neutralized with 1N HCl (2 mL). The organic layers were washed with 5 mL of 1N HCl, water, then brine. Combined organic layers were dried over magnesium sulfate, filtered, and concentrated to dryness, then dried under high vacuum to obtain the title compound as an off-white solid (1.44 g).

MS(ES): 304.9 (M+H) for C₁₀H₁₁BrN₂O₄

1H NMR (300 MHz, DMSO-d6) δ ppm 1.80 (s, 3 H) 3.40 (q, J=5.97 Hz, 2 H) 4.34 (t, J=5.93 Hz, 2 H) 8.22 (d, J=2.64 Hz, 1H) 8.46 (d, J=2.64 Hz, 1H) 13.24 (br. s., 1H)

The compound in the below table was prepared using the general method described above for Intermediate 310 and the starting material (SM) indicated.

| Ex | Compound | Data | SM |
|---|---|---|---|
| Intermediate 311 | 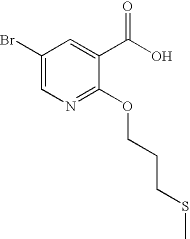 5-bromo-2-(3- (methylthio)- propoxy)nicotinic acid | MS(ES): 307.9 (M + H) for C₁₁H₁₄BrNO₃S | 3-(methylthio) propan-1-ol |

### Intermediate 312: ethyl2-(2-acetamidoethoxy)-5-bromonicotinate

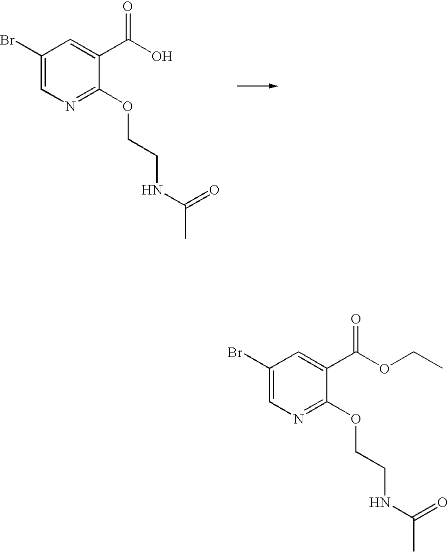

2-(2-acetamidoethoxy)-5-bromonicotinic acid, Intermediate 310 (1.44 g, 4.74 mmol) was suspended in ethanol (15.5 ml), and concentrated sulfuric acid (0.38 ml, 7.12 mmol) was added. The reaction mixture was heated at 60° C. for 3 h then stirred at rt overnight. The reaction mixture was concentrated, diluted with ethyl acetate, washed with water, brine, and dried over MgSO₄. The crude material was purified by flash chromatography (4 g, silica column, 0-8% methanol in dichlormethane over 25 min). Fractions were combined to give the title compound as a white solid (1.47 g).

MS(ES): 333.0 (M+H) for C₁₂H₁₅BrN₂O₄

1H NMR (300 MHz, DMSO-d6) δ ppm 1.25 -1.33 (m, 3H) 1.80 (s, 3H) 3.39 (q, 2H) 4.24-4.31 (m, 2H) 4.33 (t, J=5.37 Hz, 2H) 7.96 (br. s., 1H) 8.25 (d, J=2.64 Hz, 1H) 8.49 (d, J=2.45 Hz, 1H)

The compound in the below table was prepared using the general method described above for Intermediate 312 and the starting material (SM) indicated.

| Ex | Compound | Data | SM |
|---|---|---|---|
| Intermediate 313 | 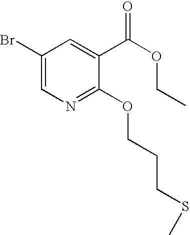 ethyl 5-bromo- 2-(3- (methylthio)- propoxy)- nicotinate | MS(ES): 335.9 (M + H) for C₁₂H₁₆BrNO₃S | 5-bromo-2-(3- (methylthio)- propoxy)- nicotinic acid Intermediate 311 |

### Intermediate 314 ethyl5-bromo-2-(3-(methylsulfonyl)propoxy)nicotinate

Ethyl 5-bromo-2-(3-(methylthio)propoxy)nicotinate (Intermediate 313, 0.4504 g, 1.35

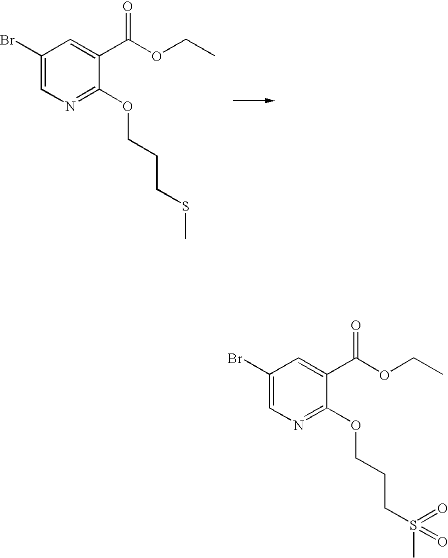

mmol) and mCPBA (0.997 g, 4.04 mmol) were suspended in dichloromethane (5.37 ml) and stirred at rt for 3 h. Then added 3 mL of dichloromethane and 0.17 g of mCPBA were added and the reaction mixture was stirred overnight. Additional 0.46 g of mCPBA were added and the mixture was stirred at rt 5 hours. The reaction mixture was concentrated in vacuo, dissolved in DCM, and filtered. The filtrate was purified by flash chromatography (12 g silica column, 0-10% methanol in dichloromethane over 30 min). Fractions were combined and dried in vacuo to obtain the title compound as a white solid (0.45 g). MS(ES): 368.0 (M+H) for C₁₂H₁₆BrNO₅S

¹H-NMR (300 MHz, DMSO-d6): δ ppm 1.30 (t, J=7.16 Hz, 3H) 2.08-2.22 (m, 2H) 3.00 (s, 3H) 3.23-3.30 (m, 2H) 4.29 (q, J=7.16 Hz, 2H) 4.42 (t, J=6.22 Hz, 2H) 8.27 (d, J=2.45 Hz, 1H) 8.50 (d, J=2.45 Hz, 1H).

The compound in the table below was prepared using the general sequence described above for the preparation of Intermediates 313 and 314 and the starting material (SM) indicated.

| Ex | Compound | Data | SM |
|---|---|---|---|
| Intermediate 314-B | 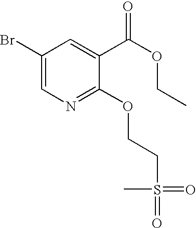 ethyl 5-bromo- 2-(2- (methylsulfonyl)- ethoxy)nicotinate | MS(ES): 352 (M + H) for C₁₁H₁₄BrNO₅S | 5-Bromo-2- chloronicotinic acid and 2-(methylthio)ethanol (followed by thiol oxidation as for Intermediate 314) |

### Intermediate 315: ethyl2-(1,3-dimethoxypropan-2-yloxy)-5-(4,4,5,5-tetramethyl-1,3,2-dioxaborolan-2-yl)nicotinate

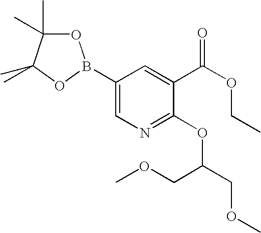

To an argon purged solution of ethyl5-bromo-2-(1,3-dimethoxypropan-2-yloxy)nicotinate (Intermediate 308, 4.5 g, 12.9 mmol) in dioxane (135 mL, 30 vol.) was added bis(pinacolato)diboron (3.93 g, 15.5 mmol) at room temperature. The reaction mixture was degassed for 15 min (argon) and was added Pd(dppf)Cl₂ (1.89 g, 2.5 mmol) followed by potassium acetate (3.8 g, 38 mmol). The reaction mixture was heated at 90° C. for 2 h. The solvent was evaporated under reduced pressure and the residue was diluted with 30% ethyl acetate in pet-ether (200 mL) and passed through neutral alumina bed. The filtrate was evaporated under reduced pressure to get the crude title compound.

The compounds in the table below were prepared using this method and the indicated starting material.

| Compound | Structure | Mass spectrum | SM |
|---|---|---|---|
| Intermediate 316 | 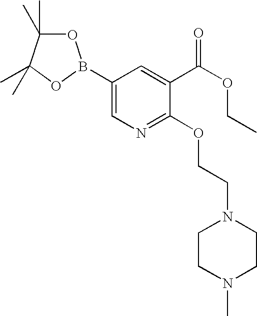 ethyl 2-(2-(4- methylpiperazin-1- yl)ethoxy)-5-(4,4,5,5- tetramethyl-1,3,2- dioxaborolan-2- yl)nicotinate | MS(ES): 420 (M + 1) for C₂₁H₃₄BN₃O₅ | Intermediate 305 ethyl 5- bromo-2-(2- (4- methylpiper- azin-1- yl)ethoxy- nicotinate |
| Intermediate 317 | 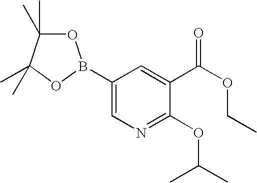 ethyl 2-isopropoxy-5- (4,4,5,5-tetramethyl- 1,3,2-dioxaborolan-2- yl)nicotinate | MS(ES): 336 (M + 1) for C₁₇H₂₆BNO₅ | Intermediate 306 ethyl 5- bromo-2- isopropoxy- nicotinate |
| Intermediate 318 | 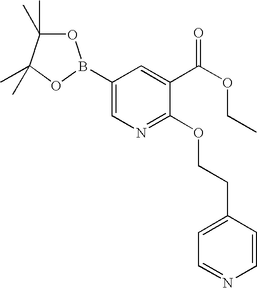 ethyl 2-(2-(pyridin-4- yl)ethoxy)-5-(4,4,5,5- tetramethyl-1,3,2- dioxaborolan-2- yl)nicotinate | MS(ES): 399 (M + 1) for C₂₁H₂₇BN₂O₅ | Intermediate 307 ethyl 5- bromo-2-(2- (pyridin-4- yl)ethoxy)- nicotinate |
| Intermediate 319 | 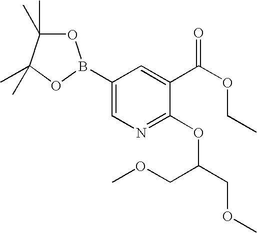 ethyl 2-(1,3- dimethoxypropan-2- yloxy)-5-(4,4,5,5- tetramethyl-1,3,2- dioxaborolan-2- yl)nicotinate | MS(ES): 396 (M + 1) for C₁₉H₃₀BNO₇ | Intermediate 308 ethyl 5- bromo-2-(1,3- dimethoxy- propan-2- yloxy)nico- tinate |
| Intermediate 320 | 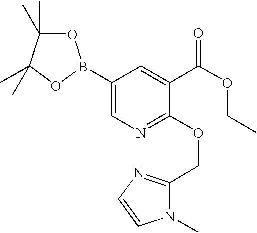 ethyl 2-((1-methyl-1H- imidazol-2- yl)methoxy)-5- (4,4,5,5-tetramethyl- 1,3,2-dioxaborolan-2- yl)nicotinate | MS(ES): 388 (M + 1) for C₁₉H₂₆BN₃O₅ | Intermediate 309 ethyl 5- bromo-2-((1- methyl-1H- imidazol-2- yl)methoxy- nicotinate |
| Intermediate 321 | 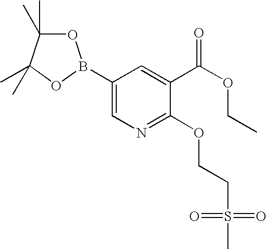 ethyl 2-(2- (methylsulfonyl)ethoxy)- 5-(4,4,5,5- tetramethyl-1,3,2- dioxaborolan-2- yl)nicotinate | MS(ES): 400 (M + H) for C₁₇H₂₆BNO₇S | Intermediate 314 ethyl 5- bromo-2-(2- (methylsul- fonyl)ethoxy)- nicotinate |
| Intermediate 321-B | 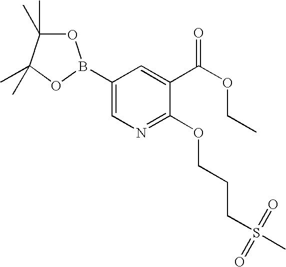 ethyl 2-(3- (methylsulfonyl)prop- poxy)-5-(4,4,5,5- tetramethyl-1,3,2- dioxaborolan-2- yl)nicotinate | MS(ES): 414.0 (M + H) for C₁₈H₂₈BNO₇S 1H NMR (300 MHz, DMSO-d6) δ ppm 1.27- 1.35 (m, 15 H) 2.10-2.23 (m, 2 H) 3.00 (s, 3 H) 3.34 (br. s., 2 H) 4.29 (q, J = 7.03 Hz, 2 H) 4.48 (t, J = 6.12 Hz, 2 H) 8.30 (d, J = 1.88 Hz, 1 H) 8.54 (d, J = 1.88 Hz, 1 H) | Intermediate 314 ethyl 5- bromo-2-(2- (methylsul- fonyl)ethoxy)- nicotinate |

### Intermediate 322: Methyl5-bromo-1-methyl-2-oxo-1,2-dihydropyridine-3-carboxylate

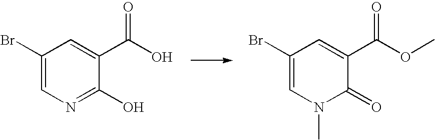

To a suspension of cesium carbonate (18.2 mmol, 5.9 g) in dry methanol (20 mL), 5-bromo-2-hydroxypyridine-3-carboxylic acid (9.2 mmol, 2 g) and iodomethane (27.3 mmol, 3.87 g) were added and heated to 80° C. for 3 h in a sealed tube. The mixture was diluted with methanol and filtered through a celite bed. The filtrate was concentrated and purified by silica gel (60-120 mesh) (product eluted with 2% Methanol in Chloroform) to yield 1.6 g of the title compound.

| | | Mass spectrum and ¹H | |
|---|---|---|---|
| Compound | Structure | NMR | SM |
| Intermediate 322 | 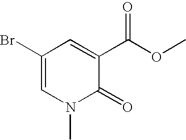 methyl 5-bromo-1- methyl-2-oxo-1,2- dihydropyridine-3- carboxylate | MS(ES): 246 (M) and 248 (M + 2) for C₈H₈BrNO₃. 300 MHz, CDCl₃: δ 3.59 (s, 3H), 3.92 (s, 3H), 7.67 (d, J = 2.85 Hz,1H), 8.19 (d, J = 2.82 Hz, 1H). | 5-bromo-2- hydroxy- pyridine-3- carboxylic acid |

### Intermediate 323: methyl1-methyl-2-oxo-5-(4,4,5,5-tetramethyl-1,3,2-dioxaborolan-2-yl)-1,2-dihydropyridine-3-carboxylate

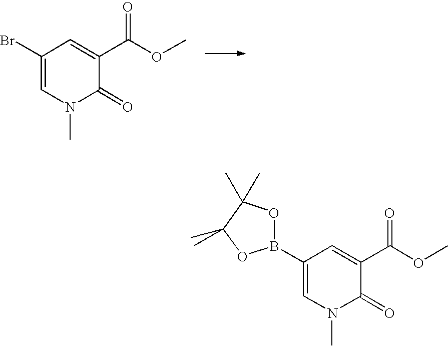

Methyl 5-bromo-1-methyl-2-oxo-1,2-dihydropyridine-3-carboxylate (Intermediate 322, 2 mmol, 0.5 g), bis(pinacolato)diboron (2.42 mmol, 0.619 g), [1,1′-bis(diphenylphosphino)ferrocene]dichloropalladium(II) (0.3 mmol, 0.244 g) and potassium acetate (6 mmol, 0.59 g) were suspended in dry dioxane (10 mL) and degassed with nitrogen for 10 min. The reaction was then heated to 100° C. for 1 h. The reaction mixture was diluted with DCM and filtered through a diatomaceous earth bed and concentrated. Then the crude mass was taken to the next step without purification. HPLC-MS analysis indicated the presence of a mixture of Boronic acid (23%) and boronate (42%).

| | | Mass spectrum and ¹H | |
|---|---|---|---|
| Compound | Structure | NMR | SM |
| Intermediate 323 | 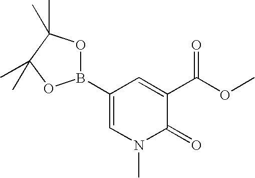 methyl 1-methyl-2- oxo-5-(4,4,5,5- tetramethyl-1,3,2- dioxaborolan-2-yl)- 1,2-dihydropyridine- 3-carboxylate | Taken to the next step as a mixture based on LCMS without further purification MS(ES): 212 (M + 1) for C₈H₁₀BNO₅ (23% as Boronic acid) and 294 (M + 1) for C₁₄H₂₀BNO₅ (42% as Boronic ester). | Intermediate 322 methyl 5- bromo-1- methyl-2- oxo-1,2- dihydro- pyridine-3- carboxylate |

The compound in the below table was prepared using the general method described above for Intermediate 323 and the starting material (SM) indicated.

| Ex | Compound | Data | SM |
|---|---|---|---|
| Intermediate 324 | 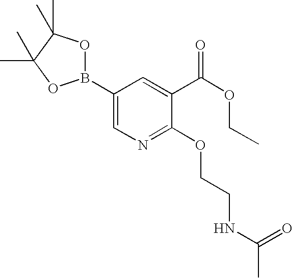 ethyl 2-(2- acetamidoethoxy)- 5-(4,4,5,5- tetramethyl- 1,3,2- dioxaborolan-2- yl)nicotinate | MS(ES): 379.2 (M + H) for C₁₈H₂₇BN₂O₆ 1H NMR (300 MHz, DMSO-d6) δ ppm 1.26-1.34 (m, 15 H) 1.80 (s, 3 H) 3.37- 3.47 (m, 2 H) 4.28 (q, J = 7.16 Hz, 2 H) 4.38 (t, J = 6.03 Hz, 2 H) 7.93 (s,1 H) 8.27 (d, J = 1.88 Hz, 1 H) 8.52 (d, J = 1.88 Hz, 1 H) | ethyl 2-(2- acetamido- ethoxy)-5- bromonicotinate Intermediate 312 |

### Intermediate 325: methyl5-{2-chloro-4-[3-(trifluoromethyl)-1H-pyrazol-1-yl]pyrimidin-5-yl}-2-methoxypyridine-3-carboxylate

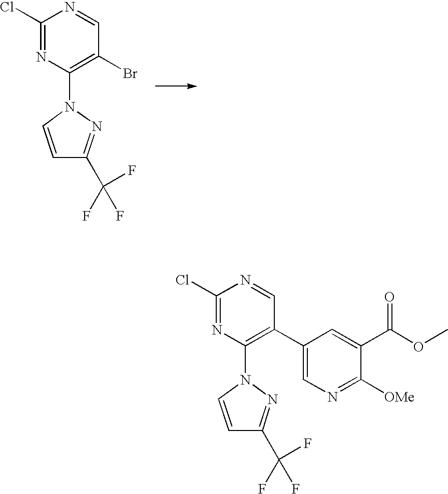

A solution of 5-bromo-2-chloro-4-[3-(trifluoromethyl)-1H-pyrazol-1-yl]pyrimidine (Intermediate 227, 2.3 mmol, 750 mg), methyl2-methoxy-5-(4,4,5,5-tetramethyl-1,3,2-dioxaborolan-2-yl)pyridine-3-carboxylate (2 3 mmol, 680 mg), [1,1′-bis(diphenylphosphino)ferrocene]dichloropalladium(II) (0.46 mmol, 340 mg) and sodium carbonate (2.3 mmol, 250 mg) in acetonitrile (10 mL)/water (10 mL) was degassed and heated to 90° C. for 20 min under nitrogen. The reaction mixture was concentrated in vacuo. The residue obtained was taken in ethyl acetate, washed with water and brine, dried over sodium sulfate and concentrated. The crude mass was purified by silica gel column chromatography (60-120 mesh) using 15% ethyl acetate/hexanes to yield 300 mg of the title compound.

| | | Mass spectrum and | |
|---|---|---|---|
| Compound | Structure | ¹H NMR | SM |
| Intermediate 325 | 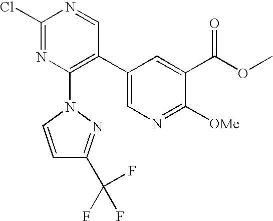 methyl 5-{2-chloro-4-[3- (trifluoromethyl)-1H- pyrazol-1-yl]pyrimidin-5- yl}-2-methoxypyridine-3- carboxylate | Taken to the next step based on UPLC without furhter purification. MS(ES): 414 (M + 1) for C₁₆H₁₁ClF₃N₅O₃. (88% pure by UPLC). | Intermediate 227 5-bromo-2- chloro-4-[3- (trifluoro- methyl)-1H- pyrazol-1- yl]pyrimidine |

### Intermediate 326: 5-bromo-2-chloro-4-[5-methyl-3-(trifluoromethyl)-1H-pyrazol-1-yl]pyrimidine

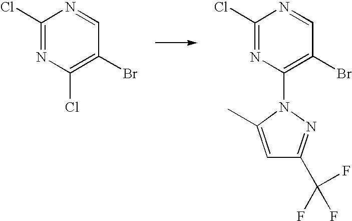

To a suspension of 5-bromo-2,4-dichloropyrimidine (44 mmol, 10 g) in acetonitrile (100 mL), was added K₂CO₃ (44 mmol, 6.1 g) and the reaction mixture was cooled to −5 to −10° C. 5-methyl-3-(trifluoromethyl)-1H-pyrazole (44 mmol, 6.6 g) was dissolved in 100 mL of acetonitrile and added dropwise. After the addition, the reaction mixture was slowly warmed to RT and stirred overnight. The reaction mixture was filtered through a celite bed and acetonitrile was removed in vacuo. The crude mass was purified by silica gel column chromatography (60-120 mesh; product eluted with 1% EtOAc/hexanes) to yield 5 g of the product.

| Compound | Structure | Mass spectrum and ¹H NMR | SM |
|---|---|---|---|
| Intermediate 326 | 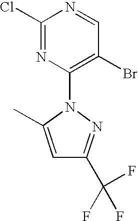 5-bromo-2-chloro- 4-[5-methyl-3- (trifluoromethyl)- 1H-pyrazol-1- yl]pyrimidine | MS(ES): 341 (M) and 343 (M + 2) for C₉H₅BrClF₃N₄. 300 MHz, CDCl₃: δ 2.52 (s, 3H), 6.53 (s, 1H), 8.94 (s, 1H). | 5-bromo-2,4- dichloro- pyrimidine and 5-methyl-3- (trifluoro- methyl)-1H- pyrazole |

### Intermediate 327: methyl5-{2-chloro-4-[5-methyl-3-(trifluoromethyl)-1H-pyrazol-1-yl]pyrimidin-5-yl}-2-methoxypyridine-3-carboxylate

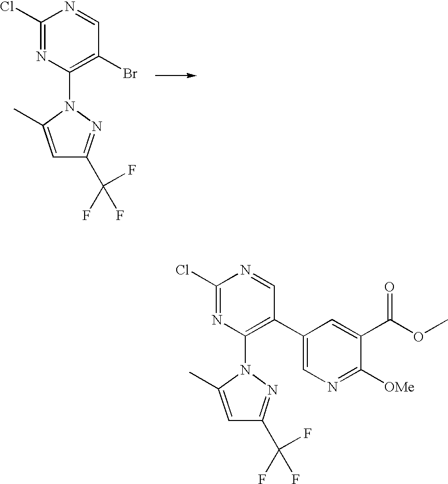

A solution of 5-bromo-2-chloro-4-[5-methyl-3-(trifluoromethyl)-1H-pyrazol-1-yl]pyrimidine (Intermediate 326, 2.05 mmol, 700 mg), methyl2-methoxy-5-(4,4,5,5-tetramethyl-1,3,2-dioxaborolan-2-yl)pyridine-3-carboxylate (2.46 mmol, 725 mg), [1,1′-bis(diphenylphosphino)ferrocene]dichloropalladium(II) (0.4 mmol, 300 mg) and sodium carbonate (2.05 mmol, 210 mg) in acetonitrile (25 mL)/water (5 mL) was degassed and heated to 90° C. for 20 min under nitrogen. The reaction mixture was concentrated in vacuo. The residue obtained was taken in ethyl acetate, washed with water and brine, dried over sodium sulfate and concentrated. The crude mass was purified by silica gel column chromatography (60-120 mesh) using 15-20% ethyl acetate/hexanes to yield 280 mg of the title compound.

| | | Mass spectrum and | |
|---|---|---|---|
| Compound | Structure | ¹H NMR | SM |
| Intermediate 327 | 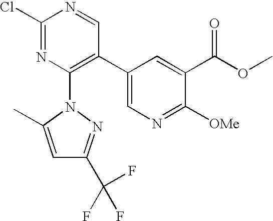 methyl 5-{2-chloro-4-[5- methyl-3- (trifluoromethyl)-1H- pyrazol-1-yl]pyrimidin-5- yl}-2-methoxypyridine-3- carboxylate | Taken to the next step based on UPLC without further purification. MS(ES): 428 (M + 1) for C₁₇H₁₃ClF₃N₅O₃. (60% pure by UPLC) | Intermediate 326 5-bromo-2- chloro-4-[5- methyl-3- (trifluoro- methyl)-1H- pyrazol-1- yl]pyrimidine |

### Intermediate 328: Ethanesulfonamide

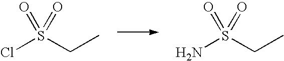

Ammonia gas was passed into a cooled THF solution (25 mL) of ethanesulfonyl chloride (19.4 mmol, 2.5 g) for 1 h. The reaction mixture was sealed and stirred at RT for 2-3 h. After completion of reaction, the reaction mixture was diluted with chloroform (20 mL) and filtered through a bed of diatomaceous earth. The filtrate was concentrated under reduced pressure and dried to get quantitative yield of the title compound as a white solid.

| | | Mass spectrum and ¹H | |
|---|---|---|---|
| Compound | Structure | NMR | SM |
| Intermediate 328 | 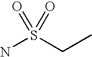 ethanesulfonamide | 400 MHz, DMSO-d₆: δ 1.21 (t, J = 7.40 Hz, 3H), 2.92 (q, J = 7.40 Hz, 2H), 6.70 (s, 2H). | ethanesulfonyl chloride |

### Intermediate 329: propane-1-sulfonamide

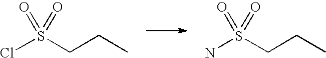

Ammonia gas was passed into a cooled THF solution (25 mL) of propane-1-sulfonyl chloride (17.5 mmol, 2.5 g) for 1 h. The reaction mixture was sealed and stirred at RT for 2-3 h. After completion of reaction, the reaction mixture was diluted with chloroform (20 mL) and filtered through a bed of diatomaceous earth. The filtrate was concentrated under reduced pressure and dried to yield the sulfonamide as a white solid.

| | | Mass spectrum and ¹H | |
|---|---|---|---|
| Compound | Structure | NMR | SM |
| Intermediate 329 | 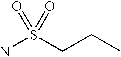 propane-1-sulfonamide | MS(ES): 123 (M) for C₃H₉NO₂S. 400 MHz, DMSO-d₆: δ 0.97 (t, J = 7.52 Hz, 3H), 1.67- 1.69 (m, 2H), 2.91-2.92 (m, 2H), 6.72 (s, 2H). | propane-1- sulfonyl chloride |

### Intermediate 330: propane-2-sulfonamide

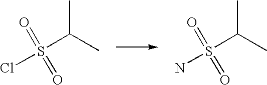

Ammonia gas was passed into a cooled THF solution (25 mL) of propane-2-sulfonyl chloride (17.5 mmol, 2.5 g) for 1 h. Then the reaction mixture was sealed and stirred at RT for 2-3 h. After completion of reaction was monitored by TLC, the reaction mixture was diluted with chloroform (20 mL), filtered through a celite bed, concentrated under reduced pressure & dried to get quantitative yield of the sulfonamide as a pale yellow thick mass.

| | | Mass spectrum and ¹H | |
|---|---|---|---|
| Compound | Structure | NMR | SM |
| Intermediate 330 | 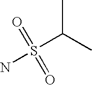 propane-2- sulfonamide | MS(ES): 123 (M) for C₃H₉NO₂S. 400 MHz, DMSO-d₆: δ 1.23 (d, J = 6.80 Hz, 6H), 2.98- 3.00 (m, 1H), 6.66 (s, 2H). | propane-2- sulfonyl chloride |

### Intermediate 331: 3-chloropropane-1-sulfonamide

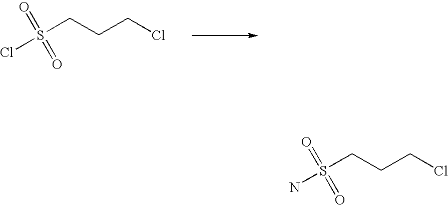

Ammonia gas was passed into a cooled THF solution (25 mL) of 3-chloropropane-1-sulfonyl chloride (14.2 mmol, 2.5 g) for 1 h. Then the reaction mixture was sealed and stirred at RT for 2-3 h. After completion of reaction, the reaction mixture was diluted with chloroform (20 mL) and filtered through celite bed, concentrated under reduced pressure and dried to get quantitative yield of the desired sulfonamide as a white solid.

| | | Mass spectrum and ¹H | |
|---|---|---|---|
| Compound | Structure | NMR | SM |
| Intermediate 331 | 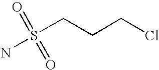 3-chloropropane-1- sulfonamide | 400 MHz, DMSO-d₆: δ 2.09- 2.11 (m, 2H), 3.09 (q, J = 5.68 Hz, 2H), 3.76 (t, J = 6.48 Hz, 2H), 6.89 (s, 2H). | 33-chloro- propane-1- sulfonyl chloride |

### Intermediate 332: 3-(morpholin-4-yl)propane-1-sulfonamide

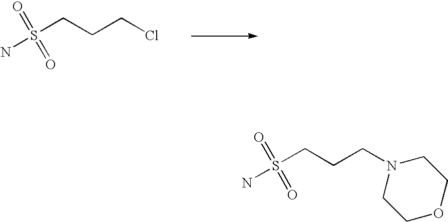

The solution of 3-chloropropane-1-sulfonamide Intermediate 331 (1.58 mmol, 0.250 g), Morpholine (1.58 mmol, 0.138 g), Na₂CO₃ (3.16 mmol, 0.335 g) and NaI (0.158 mmol, 24 mg) in dry Dioxane (5 mL) was heated to 75° C., overnight, in a sealed tube. After completion of reaction, the reaction mixture was diluted with chloroform (20 mL) and filtered through celite bed and the filtrate was concentrated under reduced pressure and dried to get the desired compound as a colorless mass (0.21 g).

| | | Mass spectrum and ¹H | |
|---|---|---|---|
| Compound | Structure | NMR | SM |
| Intermediate 332 | 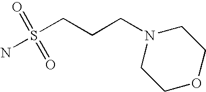 3-(morpholin-4- yl)propane-1- sulfonamide | 300 MHz, DMSO-d₆: δ 1.77- 1.79 (m, 2H), 2.35 (t, J = 6.99 Hz, 5H), 2.95-2.97 (m, 2H), 3.55 (t, J = 5.64 Hz, 5H), 6.75 (s, 2H). | Intermediate 331 3-chloro propane-1- sulfonamide |

### Intermediate 333: 4-bromo-2-methylbenzenesulfonamide

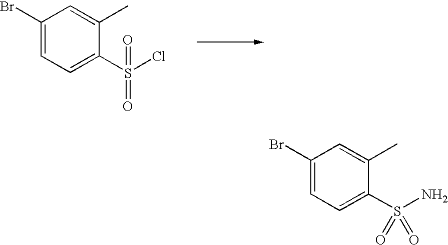

Ammonia gas was passed into a cooled THF solution (25 mL) of 4-bromo-2-methylbenzenesulfonyl chloride (9.27 mmol, 2.5 g) for 1 h. Then the reaction mixture was sealed and stirred overnight at rt. After completion of reaction, as monitored by TLC, the reaction mixture was diluted with chloroform (20 mL) and filtered through celite bed and the filtrate was concentrated under reduced pressure and dried to get quantitative yield of the desired sulfonamide as a white solid.

| | | Mass spectrum and ¹H | |
|---|---|---|---|
| Compound | Structure | NMR | SM |
| Intermediate 333 | 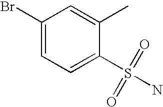 4-bromo-2- methylbenzenesulfon amide | MS(ES): 249 (M − 1) for C₇H₈BrNO₂S 400 MHz, DMSO-d₆: δ 2.58 (s, 3H), 7.50 (s, 2H), 7.60 (dd, J = 1.64, 8.40 Hz, 1H), 7.65 (s, 1H), 7.76 (d, J = 8.40 Hz, 1H). | 4-bromo-2- methylben- zenesul- fonyl chloride |

### Intermediate 334: 5-bromo-1,2-benzothiazol-3(2H)-one1,1-dioxide

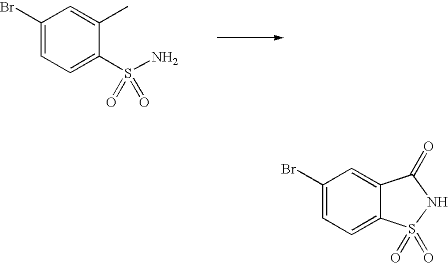

A suspension of 4-bromo-2-methylbenzenesulfonamide (Intermediate 333, 9.6 mmol, 2.4 g), periodic acid (76.8 mmol, 17.5 g), Chromium oxide (4.8 mmol, 0.047 g) in dry acetonitrile (25 mL) was heated to reflux for 3 h. Isopropyl alcohol (5 mL) was added slowly and the reaction mixture was heated to reflux for another 10 min. Then the reaction mixture was cooled to rt and it was filtered and washed with acetone (10 mL×3). The filtrate was concentrated and triturated with 10 mL of 2 N H₂SO₄ and filtered to get 1.5 g of the title compound as an off-white solid.

| | | Mass spectrum and ¹H | |
|---|---|---|---|
| Compound | Structure | NMR | SM |
| Intermediate 334 | 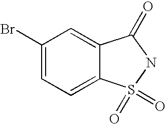 5-bromo-1,2- benzothiazol-3(2H)- one 1,1-dioxide | MS(ES): 262 (M) and 264 (M + 2) for C₇H₄BrNO₃S 400 MHz, DMSO-d₆: δ 8.06 (d, J = 1.52 Hz, 1H), 8.08 (d, J = 1.44 Hz, 1H), 8.11-8.15 (m, 1H). | Intermediate 333 4-bromo-2- methylben- zenesul- fonamide |

### Intermediate 335: (1,1-dioxido-3-oxo-2,3-dihydro-1,2-benzothiazol-5-yl)boronic acid

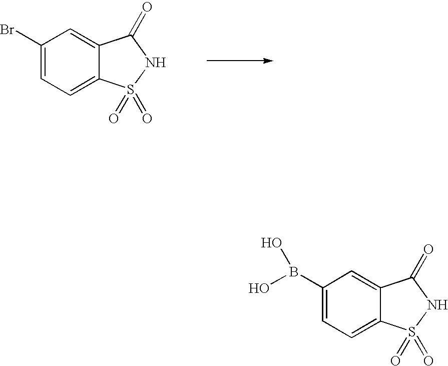

5-bromo-1,2-benzothiazol-3(2H)-one 1,1-dioxide (Intermediate 334, 0.954 mmol, 0.25 g), bis(pinacolato)diboron (2.862 mmol, 0.726 g), [1,1′-bis(diphenylphosphino)ferrocene]dichloropalladium(II) (0.0954 mmol, 0.077 g) and potassium acetate (2.862 mmol, 0.28 g) were suspended in dry DMSO (5 mL) and degassed with nitrogen for 10 min. The reaction was then subjected to microwave condition at 100° C. for 30 min. The reaction mixture was concentrated under vacuum. The residue obtained was washed with hexane (5 mL), decanted and dried to give the product. Taken to the next step without purification.

| | | Mass spectrum and ¹H | |
|---|---|---|---|
| Compound | Structure | NMR | SM |
| Intermediate 335 | 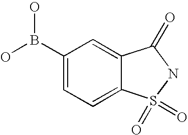 (1,1-dioxo-3-oxo- 2,3-dihydro-1,2- benzothiazol-5- yl)boronic acid | Taken to the next step based on LCMS without further purification MS(ES): 226 (M − 1) for C₇H₆BNO₅S. (78% pure by LCMS) | Intermediate 334 5-bromo- 1,2-benzo- thiazol- 3(2H)-one 1,1-dioxide |

### Intermediate 336: N-(4-methyl-5-sulfamoyl-1,3-thiazol-2-yl)acetamide

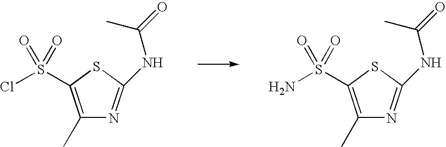

Ammonia gas was passed into a cooled THF solution (2 mL) of 2-(acetylamino)-4-methyl-1,3-thiazole-5-sulfonyl chloride (0.39 mmol, 0.1 g) for 1 h. Then the reaction mixture was sealed and stirred at RT for 2-3 h. After completion of reaction, the reaction mixture was diluted with chloroform (20 mL) and filtered through a celite bed. The filtrate was concentrated under reduced pressure and dried to get quantitative yield of an off-white solid.

| | | Mass spectrum and ¹H | |
|---|---|---|---|
| Compound | Structure | NMR | SM |
| Intermediate 336 | 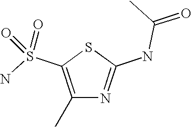 N-(4-methyl-5- sulfamoyl-1,3- thiazol-2- yl)acetamide | MS(ES): 236 (M + 1) for C₆H₉N₃O₃S₂. 300 MHz, DMSO-d₆: δ 2.15 (d, J = 5.07 Hz, 3H), 2.36 (s, 3H), 7.61 (s, 2H), 12.40 (s, 1H). | 2- (acetylamino)- 4- methyl-1,3- thiazole-5- sulfonyl chloride |

### Intermediate 337: 2,2,2-trifluoroethanesulfonamide

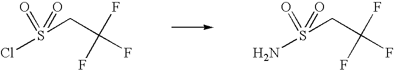

Ammonia gas was passed into a cooled THF solution (2 mL) of 2,2,2-trifluoroethanesulfonyl chloride (5.48 mmol, 1 g) for 1 h. Then the reaction mixture was sealed and stirred at RT for 2 h. Completion of reaction was monitored by TLC. The reaction mixture was diluted with dichloromethane (50 mL) and filtered through celite bed and the filtrate was concentrated under reduced pressure and dried to get 800 mg of the title compound as a white solid.

| | | Mass spectrum and ¹H | |
|---|---|---|---|
| Compound | Structure | NMR | SM |
| Intermediate 337 | 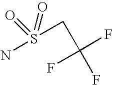 2,2,2- trifluoroethanesulfonamide | 400 MHz, DMSO-d₆: δ 4.21- 4.29 (m, 2H), 7.49 (s, 2H). | 2,2,2- trifluoro- ethane- sulfonyl chloride |

### Intermediate 338: 3,5-dimethyl-1,2-oxazole-4-sulfonamide

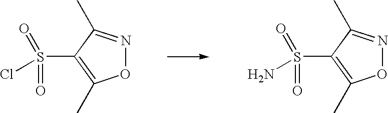

Ammonia gas was passed into a cooled THF solution (2 mL) of 3,5-dimethyl-1,2-oxazole-4-sulfonyl chloride (0.76 mmol, 150 mg) for 1 h. Then the reaction mixture was sealed and stirred at RT for 2-3 h. The reaction mixture was diluted with chloroform (20 mL) and filtered through celite bed and the filtrate was concentrated under reduced pressure and dried to get 120 mg of the title compound as a white solid.

| | | Mass spectrum and ¹H | |
|---|---|---|---|
| Compound | Structure | NMR | SM |
| Intermediate 338 | 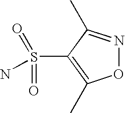 3,5-dimethyl-1,2- oxazole-4- sulfonamide | MS(ES): 177 (M + 1) for C₅H₈N₂O₃S. | 3,5- dimethyl- 1,2-oxazole- 4-sulfonyl chloride |

### Intermediate 339: 2,4-dimethyl-1,3-thiazole-5-sulfonamide

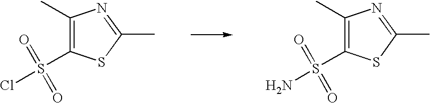

Ammonia gas was passed into a cooled THF solution (25 mL) of 2,4-dimethyl-1,3-thiazole-5-sulfonyl chloride (0.47 mmol, 100 mg) for 1 h. Then the reaction mixture was sealed and stirred at RT for 2 h. The reaction mixture was diluted with chloroform (20 mL) and filtered through celite bed and the filtrate was concentrated under reduced pressure and dried to get 90 mg of the desired sulfonamide as a white solid.

| | | Mass spectrum and ¹H | |
|---|---|---|---|
| Compound | Structure | NMR | SM |
| Intermediate 339 | 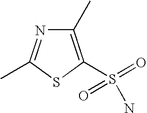 2,4-dimethyl-1,3- thiazol-5- sulfonamide | MS(ES): 193 (M + 2) for C₅H₈N₂O₂S₂. (91% pure by UPLC) | 2,4- dimethyl- 1,3-thiazole- 5-sulfonyl chloride |

### Intermediate 340: 1-(methylsulfonyl)methanesulfonamide

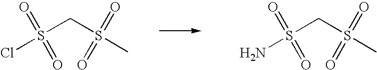

Ammonia gas was passed into a cooled THF solution (2 mL) of (methylsulfonyl)methanesulfonyl chloride (1.1 mmol, 200 mg) for 1 h. Then the reaction mixture was sealed and stirred at RT for 2 h. After completion of reaction, as monitored by TLC, the reaction mixture was diluted with chloroform (20 mL) and filtered through celite bed and the filtrate was concentrated under reduced pressure and dried to get 65 mg of white solid.

| | | Mass spectrum and ¹H | |
|---|---|---|---|
| Compound | Structure | NMR | SM |
| Intermediate 340 | 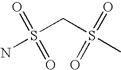 1- (methylsulfonyl)- methanesulfonamide | MS(ES): 174 (M + 1) for C₂H₇NO₄S₂. 300 MHz, DMSO-d₆: δ 3.16 (s, 3H), 4.96 (s, 1H), 7.38 (s, 1H). | (methylsul- fonyl)- methane- sulfonyl chloride |

### Intermediate 341: N-(4-methoxybenzyl)-1-methyl-1H-imidazole-4-sulfonamide

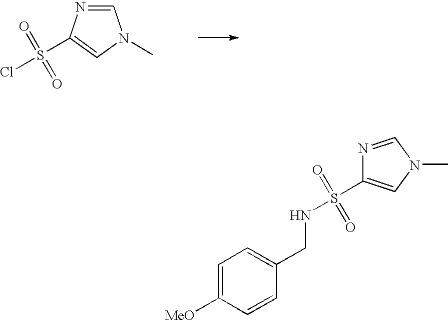

To a solution of 1-methyl-1H-imidazole-4-sulfonyl chloride (4.4 mmol, 0.8 g) in dry CH₂Cl₂ (10 mL), was added 4-methoxybenzylamine (3.5 mmol, 0.49 g) and Et₃N (1.33 mmol, 1.34 g) and left to stir overnight at RT. The reaction mixture was diluted with dichloromethane (20 mL) and water. The organic layer was separated, dried over Na₂SO₄ and concentrated. The solid that was obtained was further recrystallised from CHCl₃ and petroleum ether to get 500 mg of the desired protected sulfonamide.

| | | | |
|---|---|---|---|
| | | Mass spectrum and ¹H/ | |
| Compound | Structure | NMR | SM |

| | | | |
|---|---|---|---|
| Intermediate 341 | 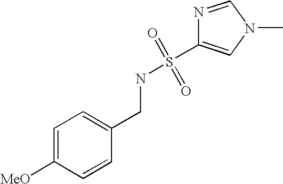 N-(4- methoxybenzyl)-1- methyl-1H- imidazole-4- sulfonamide | MS(ES): 282 (M + 1) for C₁₂H₁₅N₃O₃S. 400 MHz, DMSO-d₆: δ 3.67 (s, 3H), 3.71 (s, 3H), 3.94 (d, J = 6.28 Hz, 2H), 6.82-6.85 (m, 2H), 7.16 (d, J = 8.60 Hz, 2H), 7.67 (d, J = 1.20 Hz, 1H), 7.76 (s, 1H), 7.87 (t, J = 6.28 Hz, 1H). | 1-methyl- 1H- imidazole- 4-sulfonyl chloride |

### Intermediate 342: 1-methyl-1H-imidazole-4-sulfonamide

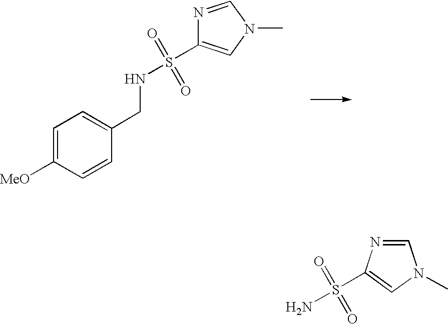

To a cooled solution of N-(4-methoxybenzyl)-1-methyl-1H-imidazole-4-sulfonamide (Intermediate 341, 1.06 mmol, 0.3 g), was added TFA (15 mL) and the reaction mixture was stirred at 0° C. for 1 hour. The mixture was concentrated and methanol was added to the residue and further concentrated to get 150 mg of the desired sulfonamide.

| | | Mass spectrum and ¹H | |
|---|---|---|---|
| Compound | Structure | NMR | SM |
| Intermediate 342 | 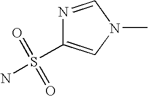 1-methyl-1H- imidazole-4- sulfonamide | MS(ES): 162 (M + 1) for C₄H₇N₃O₂S. 400 MHz, DMSO-d₆: δ 3.69 (s, 3H), 7.13 (s, 2H), 7.61 (d, J = 0.88 Hz, 1H), 7.74 (s, 1H). | Intermediate 341 N-(4- methoxy- benzyl)-1- methyl-1H- imidazole- 4- sulfonamide |

### Intermediate 343: 2,5-dihydrothiophene-3-sulfonamide1,1-dioxide

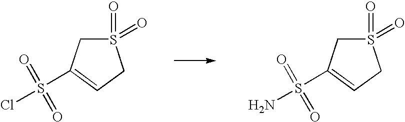

Ammonia gas was passed into a cooled THF solution (25 mL) of 2,5-dihydrothiophene-3-sulfonyl chloride 1,1-dioxide (0.93 mmol, 200 mg) for around 20 min. Then the reaction mixture was sealed and stirred at RT for 2-3 h. The reaction mixture was diluted with ethyl acetate (20 mL) and filtered. The filtrate was concentrated under reduced pressure and dried to get quantitative yield of the desired sulfonamide as a white solid.

| | | Mass spectrum and ¹H | |
|---|---|---|---|
| Compound | Structure | NMR | SM |
| Intermediate 343 | 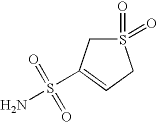 2,5- dihydrothiophene-3- sulfonamide 1,1- dioxide | 400 MHz, DMSO-d₆: δ 4.11 (s, 2H), 4.21 (d, J = 1.60 Hz, 2H), 6.74 (s, 1H), 7.44-7.48 (m, 2H). | 2,5- dihydrothio phene-3- sulfonyl chloride 1,1-dioxide |

### Intermediate 344: N-(4-methoxybenzyl)-6-methyl-2,4-dioxo-1,2,3,4-tetrahydropyrimidine-5-sulfonamide

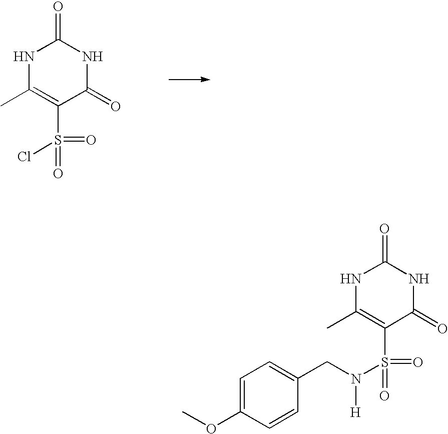

To a solution of 6-methyl-2,4-dioxo-1,2,3,4-tetrahydropyrimidine-5-sulfonyl chloride (2.23 mmol, 500 mg) in dry CH₂Cl₂ (10 mL), was added 4-methoxybenzylamine (1.78 mmol, 244 mg) and Et₃N (11.6 mmol, 1.13 g) and left to stir overnight at RT. The reaction mixture was diluted with dichloromethane (20 mL) and water. The organic layer was separated and the precipitate formed in aqueous layer was filtered and dried to get 350 mg of the product.

| | | Mass spectrum and ¹H | |
|---|---|---|---|
| Compound | Structure | NMR | SM |
| Intermediate 344 | 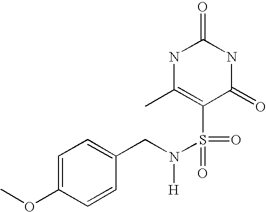 N-(4-methoxybenzyl)- 6-methyl-2,4-dioxo- 1,2,3,4- tetrahydropyrimidine- 5-sulfonamide | MS(ES): 326 (M + 1) for C₁₃H₁₅N₃O₅S. 400 MHz, DMSO-d₆: δ 2.31 (s, 3H), 3.70 (s, 3H), 3.99 (d, J = 6.48 Hz, 2H), 6.79 (d, J = 8.64 Hz, 2H), 7.17 (d, J = 8.60 Hz, 2H), 7.23 (t, J = 6.56 Hz, 1H), 11.32 (br s, 2H). | 6-methyl- 2,4-dioxo- 1,2,3,4- tetrahydro- pyrimidin-5- sulfonyl chloride |

### Intermediate 345: 6-methyl-2,4-dioxo-1,2,3,4-tetrahydropyrimidine-5-sulfonamide

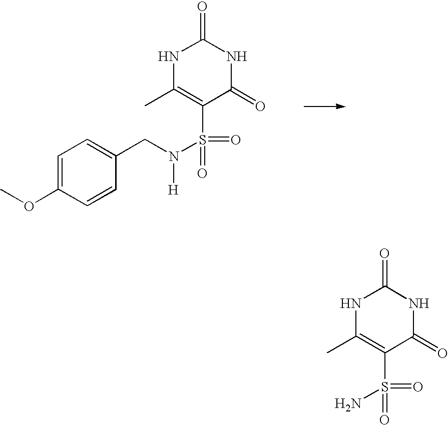

To a cooled solution of N-(4-methoxybenzyl)-6-methyl-2,4-dioxo-1,2,3,4-tetrahydropyrimidine-5-sulfonamide (Intermediate 344, 1.07 mmol, 350 mg), was added TFA (10 mL) and the reaction mixture was stirred at rt for 6 h. The mixture was concentrated and methanol was added to the residue and further concentrated to get 200 mg of the desired sulfonamide.

| | | Mass spectrum and ¹H | |
|---|---|---|---|
| Compound | Structure | NMR | SM |
| Intermediate 345 | 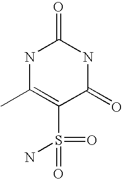 6-methyl-2,4-dioxo- 1,2,3,4-tetrahydro- pyrimidin-5- sulfonamide | MS(ES): 206 (M + 1) for C₅H₇N₃O₄S. 300 MHz, DMSO-d₆: δ 2.40 (s, 3H), 6.81 (d, J = 12.72 Hz, 2H), 11.42 (br s, 1H), 11.60 (br s, 1H). | Intermediate 344 N-(4- methoxy- benzyl)-6- methyl-2,4- dioxo-1,2,3,4- tetrahydro- pyrimidine-5- sulfonamide |

### Intermediate 346: 1,3,5-trimethyl-1H-pyrazole-4-sulfonamide

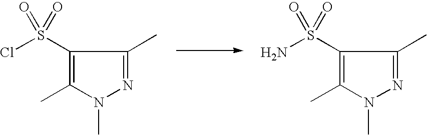

Ammonia gas was passed into a cooled THF solution (25 mL) of 1,3,5-trimethyl-1H pyrazole-4-sulfonyl chloride (0.96 mmol, 0.2 g) for around 1 h. Then the reaction mixture was sealed and stirred at RT for 2-3 h. After completion of reaction, the reaction mixture was diluted with chloroform (15 mL) and filtered through celite bed. The filtrate was concentrated under reduced pressure and dried to get the title compound as a white solid (149 mg).

| | | Mass spectrum and ¹H | |
|---|---|---|---|
| Compound | Structure | NMR | SM |
| Intermediate 346 | 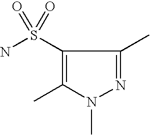 1,3,5-trimethyl-1H- pyrazol-4- sulfonamide | MS (ES): 190 (M + 1) for C₆H₁₁N₃O₂S. 400 MHz, DMSO-d₆: δ 2.23 (s, 3H), 2.36 (s, 3H), 3.65 (s, 3H), 7.03 (s, 2H). | 1,3,5- trimethyl- 1H- pyrazole-4- sulfonyl chloride |

### Intermediate 347: 3-methyl-2-oxo-2,3-dihydro-1,3-benzoxazole-6-sulfonamide

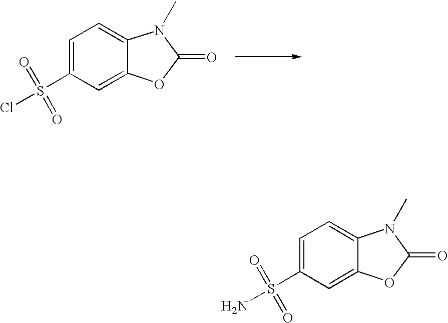

Ammonia gas was passed into a cooled THF solution (25 mL) of 3-methyl-2-oxo-2,3-dihydro-1,3-benzoxazole-6-sulfonyl chloride (0.5 mmol, 0.125 g) for 1 h and sealed. Then the reaction mixture was stirred at RT for 2-3 h. The reaction mixture was diluted with chloroform (20 mL) and filtered through celite bed. The filtrate was concentrated under reduced pressure and dried to get quantitative yield of the title compound as an off-white solid.

| | | Mass spectrum and ¹H | |
|---|---|---|---|
| Compound | Structure | NMR | SM |
| Intermediate 347 | 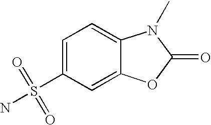 3-methyl-2-oxo-2,3- dihydro-1,3- benzoxazole-6- sulfonamide | MS(ES): 227 (M − 1) for C₈H₈N₂O₄S. 400 MHz, DMSO-d₆: δ 3.36 (s, 3H), 7.37 (s, 2H), 7.39- 7.42 (m, 1H), 7.70-7.73 (m, 2H). | 3-methyl-2- oxo-2,3- dihydro-1,3- benzoxazole- 6-sulfonyl chloride |

### Intermediate 348: 3-acetylbenzenesulfonamide

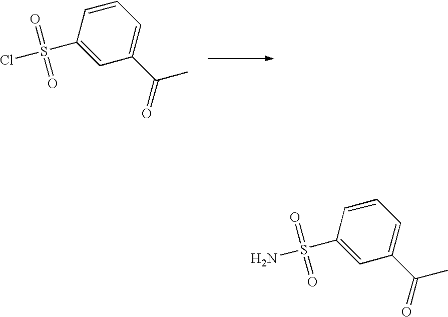

Ammonia gas was passed into a cooled solution (25 mL) of 3-acetylbenzenesulfonyl chloride (2.2 mmol, 500 mg) in dry 1,4-dioxane(15 mL) for 20 min and sealed. Then the reaction mixture was stirred at RT for 2 h. It was then diluted with ethyl acetate (20 mL) and filtered. The filtrate was concentrated under reduced pressure and dried to get the desired sulfonamide as a white solid in quantitative yield.

| | | Mass spectrum and ¹H | |
|---|---|---|---|
| Compound | Structure | NMR | SM |
| Intermediate 348 | 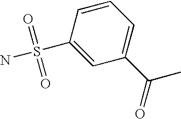 3-acetylbenzene- sulfonamide | MS(ES): 200 (M + 1) for C₈H₉NO₃S. | 3- acetylben- zenesul- fonyl chloride |

### Intermediate 349: 1-methyl-3-(trifluoromethyl)-1H-pyrazole-4-sulfonamide

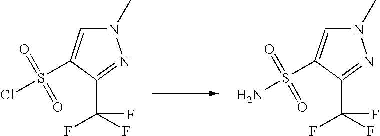

Ammonia gas was passed into a cooled THF solution (25 mL) of 1-methyl-3-(trifluoromethyl)-1H-pyrazole-4-sulfonyl chloride (0.5 mmol, 0.125 g) for 1 h. Then the reaction mixture was sealed and stirred at RT for 2-3 h. After completion of reaction, the reaction mixture was diluted with chloroform (20 mL) and filtered through celite bed. The filtrate was concentrated under reduced pressure and dried to get quantitative yield of the title compound as a white solid.

| | | Mass spectrum and ¹H | |
|---|---|---|---|
| Compound | Structure | NMR | SM |
| Intermediate 349 | 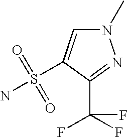 1-methyl-3- (trifluoromethyl)-1H- pyrazole-4- sulfonamide | MS(ES): 228 (M − 1) for C₅H₆F₃N₃O₂S. 400 MHz, DMSO-d₆: δ 3.95 (s, 3H), 7.62 (s, 2H), 8.36 (s, 1H). | 1-methyl-3- (trifluoro- methyl)-1H- pyrazol-4- sulfonyl chloride |

### Intermediate 350: N-{4-[(4-methoxybenzyl)sulfamoyl]benzyl}acetamide

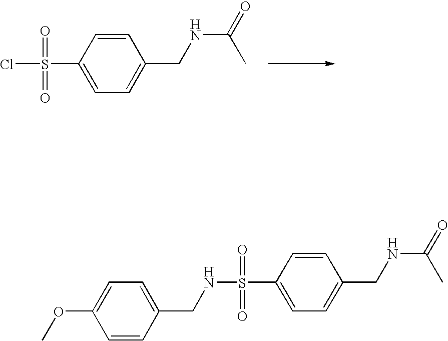

To a cooled solution of 4-methoxybenzylamine (4.24 mmol, 0.581 g) and Et₃N (0.99 mL, 7.07 mmol) in dry dichloromethane, was added 4-┌(acetylamino)methyl┐benzenesulfonyl chloride (2.83 mmol, 0.7 g) and the reaction mixture was stirred overnight at RT. The reaction mixture was diluted with chloroform (20 mL) and the organic layer was washed with 10% citric acid solution (50 mL), 10% sodium bicarbonate solution (50 mL) and brine (25 mL). The organic layer was dried over sodium sulfate and concentrated under reduced pressure and dried to get 0.2 g of the protected sulfonamide as an off-white solid.

| | | Mass spectrum and ¹H | |
|---|---|---|---|
| Compound | Structure | NMR | SM |
| Intermediate 350 | 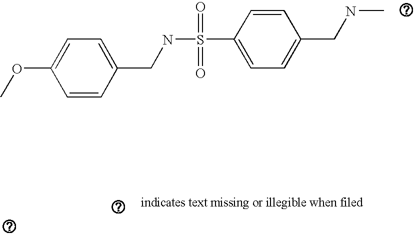 N-{4-[(4- methoxybenzyl)sul- famoyl]benzyl}- acetamide | MS(ES): 349 (M + 1) for C₁₇H₂₀N₂O₄S. 400 MHz, DMSO-d₆: δ 1.91 (s, 3H), 3.72 (s, 3H), 3.88 (d, J = 6.08 Hz, 2H), 4.33 (d, J = 5.84 Hz, 2H), 6.85 (d, J = 8.56 Hz, 2), 7.14 (d, J = 8.52 Hz, 2H), 7.44 (d, J = 8.12 Hz, 2H), 7.75 (d, J = 8.20 Hz, 2H), 8.02 (t, J = 6.12 Hz, 1H), 8.46 (t, J = 9.32 Hz, 1H). | 4- [(acetylamino)- methyl]- benzenesul- fonyl chloride |

### Intermediate 351: N-(4-sulfamoylbenzyl)acetamide

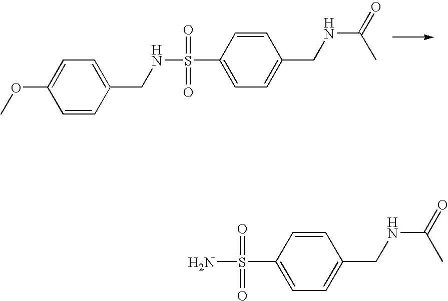

To a cooled solution of N-{4-[(4-methoxybenzyl)sulfamoyl]benzyl}acetamide (PE-66-14-I, 0.56 mmol, 195 mg), was added TFA (10 mL) and the reaction mixture was stirred overnight at rt. The mixture was concentrated and methanol was added to the residue and further concentrated to get 200 mg of the desired sulfonamide as a pale brown solid which was taken to the next step without further purification.

| | | Mass spectrum and ¹H | |
|---|---|---|---|
| Compound | Structure | NMR | SM |
| Intermediate 351 | 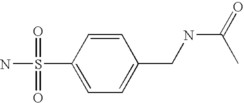 N-(4- Sulfamoylbenzyl) acetamide | MS(ES): 229 (M + 1) for C₉H₁₂N₂O₃S. 300 MHz, DMSO-d₆: δ 1.87 (s, 3H), 4.29 (d, J = 5.79 Hz, 2H), 7.30 (s, 2H), 7.40 (d, J = 7.92 Hz, 2H), 7.75 (d, J = 7.53 Hz, 2H). | Intermediate 350 N-{4-[(4- methoxy- benzyl)- sulfam- oyl]- benzyl}- acetamide |

### Intermediate 352: 1,3-dimethyl-2,4-dioxo-1,2,3,4-tetrahydropyrimidine-5-sulfonamide

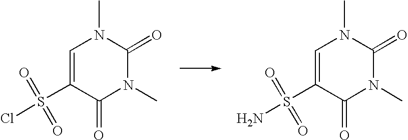

Ammonia gas was passed into a cooled THF solution (25 mL) of 1,3-dimethyl-2,4-dioxo-1,2,3,4-tetrahydropyrimidine-5-sulfonyl chloride (0.53 mmol, 0.125 g) for 1 h and sealed. Then the reaction mixture was stirred at RT for 2-3 h. After completion of reaction, the reaction mixture was diluted with chloroform (20 mL) and filtered through celite bed. The filtrate was concentrated under reduced pressure and dried to get quantitative yield of the title compound as a white solid.

| | | Mass spectrum and ¹H | |
|---|---|---|---|
| Compound | Structure | NMR | SM |
| Intermediate 352 | 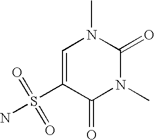 1,3-Dimethyl-2,4- dioxo-1,2,3,4- tetrahydropyrimidine- 5-sulfonamide | MS(ES): 220 (M + 1) for C₆H₉N₃O₄S. 300 MHz, DMSO-d₆: δ 3.19 (s, 3H), 3.39 (s, 3H), 7.07 (s, 2H), 8.40 (s, 1H). | 1,3- dimethyl- 2,4-dioxo- 1,2,3,4- tetrahydro- pyrimidin-5- sulfonyl chloride |

### Intermediate 353: 2-(2,5-dioxopyrrolidin-1-yl)ethanesulfonamide

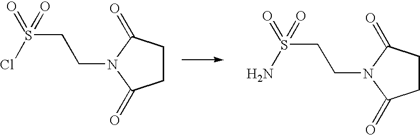

Ammonia gas was passed into a cooled solution (25 mL) of 2-(2,5-dioxopyrrolidin-1-yl)ethanesulfonyl chloride (1.77 mmol, 400 mg) in dry 1,4-dioxane (10 mL) for 20 min and sealed. Then the reaction mixture was stirred at RT for 2 h. It was then diluted with ethyl acetate (20 mL) and filtered. The filtrate was concentrated under reduced pressure and dried to get 150 mg of the desired sulfonamide as a white solid.

| | | Mass spectrum and ¹H | |
|---|---|---|---|
| Compound | Structure | NMR | SM |
| Intermediate 353 | 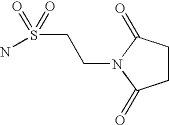 2-(2,5-dioxopyrrolidin- 1- yl)ethanesulfonamide | 300 MHz, DMSO-d₆: δ 2.57 (d, J = 11.43 Hz, 4H), 3.13- 3.18 (m, 2H), 3.69-3.74 (m, 2H), 7.00 (s, 2H). | 2-(2,5- dioxopyrrolidin- 1- yl)ethane- sulfonyl chloride |

### Intermediate 354: 1H-pyrazole-4-sulfonamide

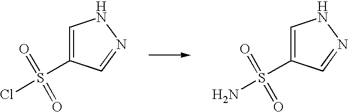

Ammonia gas was passed into a cooled THF solution (25 mL) of 1H-pyrazole-4-sulfonyl chloride (0.75 mmol, 0.125 g) for 1 h and sealed. Then the reaction mixture was stirred at RT for 2-3 h. After completion of reaction, the reaction mixture was diluted with chloroform (20 mL) and filtered through celite bed. The filtrate was concentrated under reduced pressure and dried to get quantitative yield of the title compound as a white solid.

| | | Mass spectrum and ¹H | |
|---|---|---|---|
| Compound | Structure | NMR | SM |
| Intermediate 354 | 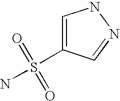 1H-pyrazole-4- sulfonamide | MS(ES): 148 (M + 1) for C₃H₅N₃O₂S. | 1H- pyrazole-4- sulfonyl chloride |

### Intermediate 355: 5-Bromo-2-hydroxypyridine-3-carboxylic acid

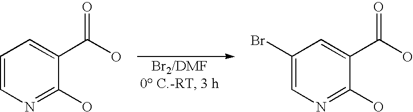

To a solution of 2-hydroxypyridine-3-carboxylic acid (35.94 mmol, 5 g) in dry DMF cooled with an ice-water bath, a solution of bromine (57.51 mmol, 3 mL, 9.19 g in cooled DMF) was added dropwise at 0° C. over 1 h. The reaction mixture was stirred at rt for 3 h and then quenched with ice-water. The resulting yellow solid was filtered, washed with water and dried under vacuum to get 6 g of the title compound.

| Compound | Structure | Mass spectrum and ¹H NMR | SM |
|---|---|---|---|
| Intermediate 355 | 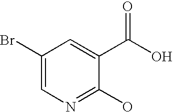 5-Bromo-2- hydroxypyridine- 3-carboxylic acid | MS (ES): 218 (M) and 220 (M + 2) for C₆H₄BrNO₃. 400 MHz, DMSO-d₆: δ 8.26 (d, J = 3.56 Hz, 1H), 8.34 (d, J = 3.68 Hz, 1H), 13.70 (s, 2H). | 2-Hydroxy pyridine-3- carboxylic acid |

### Intermediate 356: 5-bromo-2-chloropyridine-3-carboxylic acid

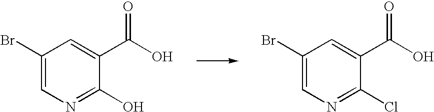

A solution of 5-bromo-2-hydroxypyridine-3-carboxylic acid (Intermediate 355, 26.83 mmol, 5.85 g) in POCl₃ (14.63 mL, 2.5 v/w) was heated to reflux for 12 h. It was cooled to RT and POCl₃ was removed in vacuo. Then ice water was added to the reaction mixture and extracted with EtOAc. The EtOAc layer was concentrated to get a yellow solid. The solid obtained was washed with chilled CHCl₃ to yield the title compound (2.86 g).

| | | Mass spectrum and ¹H | |
|---|---|---|---|
| Compound | Structure | NMR | SM |
| Intermediate 356 | 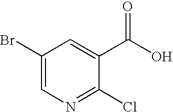 5-Bromo-2- chloropyridine-3- carboxylic acid | MS(ES): 237 (M + 1) for C₆H₃BrClNO₂. 400 MHz, DMSO-d₆: δ 8.44 (d, J = 2.52 Hz, 1H), 8.72 (d, J = 2.48 Hz, 1H), 13.91 (s, 1H). | Intermediate 355 5-Bromo-2- hydroxy- pyridine-3- carboxylic acid |

### Intermediate 357: 5-bromo-2-(methylsulfanyl)pyridine-3-carboxylic acid

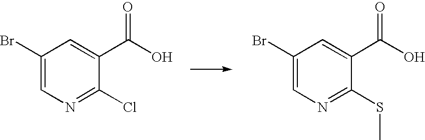

To a solution of 5-bromo-2-chloropyridine-3-carboxylic acid (Intermediate 356, 10.57 mmol, 2.5 g) in dioxane was added 21% aqueous solution of NaSMe (8.8 mL, 26.43 mmol, 1.85 g) and the mixture was heated in a sealed tube at 110° C. for 2 h. After completion of the reaction, the crude mass was dissolved in water and acidified with 10% citric acid solution and the solid obtained was filtered and dried to yield the product (2 g).

| | | Mass spectrum and ¹H | |
|---|---|---|---|
| Compound | Structure | NMR | SM |
| Intermediate 357 | 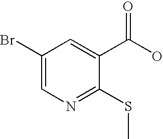 5-Bromo-2- (methylsulfanyl)- pyridine-3- carboxylic acid | MS(ES): 248 (M) and 250 (M + 2) for C₇H₆BrNO₂S. 400 MHz, DMS)-d₆: δ 2.41 (s, 3H), 8.29 (d, J = 3.16 Hz, 1H), 8.77 (d, J = 3.16 Hz, 1H), 13.70 (s, 1H). | Intermediate 356 5-Bromo-2- chloro- pyridine-3- carboxylic acid |

### Intermediate 358: Methyl 5-bromo-2-(methylsulfanyl)pyridine-3-carboxylate

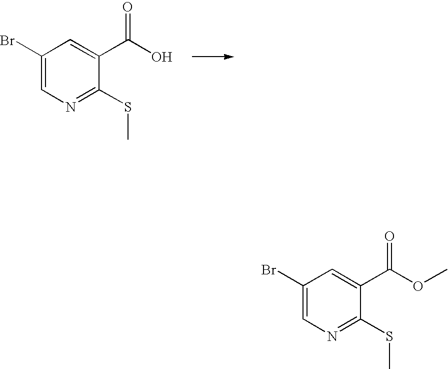

To a suspension of 5-bromo-2-(methylsulfanyl)pyridine-3-carboxylic acid (Intermediate 357, 7.66 mmol, 1.9 g) in MeOH (20 ml) at 0° C., was slowly added thionyl chloride (15.32 mmol, 1.82 g). After the addition was complete, the reaction mixture was refluxed for 3 h. The solvent was concentrated in vacuo and the crude mixture was taken in EtOAc (30 mL), washed with aq. NaHCO₃ solution, water and brine, dried over Na₂SO₄, filtered and concentrated to obtain 1.6 g of the title compound.

| | | Mass spectrum and ¹H | |
|---|---|---|---|
| Compound | Structure | NMR | SM |
| Intermediate 358 | 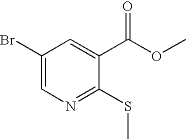 Methyl 5-bromo-2- (methylsulfanyl)- pyridine-3- carboxylate | MS(ES): 262 (M) and 262 (M + 2) for C₈H₈BrNO₂S. 400 Mhz, DMSO-d₆: δ 2.44 (s, 3H), 3.85 (s, 3H), 8.33 (d, J = 3.20 Hz, 1H), 8.81 (d, J = 3.20 Hz, 1H). | Intermediate 357 5-Bromo-2- (methylsul- fanyl)- pyridine- 3- carboxylic acid |

### Intermediate 359: methyl 2-(methylsulfanyl)-5-(4,4,5,5-tetramethyl-1,3,2-dioxaborolan-2-yl)pyridine-3-carboxylate

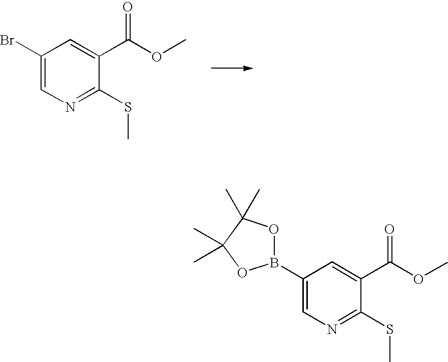

A suspension of methyl 5-bromo-2-(methylsulfanyl)pyridine-3-carboxylate (Intermediate 358, 5.7 mmol, 1.5 g), bis(pinacolato)diboron (6.2 mmol, 1.59 g), [1,1′-bis(diphenylphosphino)ferrocene]dichloropalladium(II) complex with dichloromethane (0.57 mmol, 0.467 g) and potassium acetate (17.17 mmol, 1.685 g) was taken in dioxane (5 mL) and degassed for 10 min. Then the reaction mixture was heated to 100° C. for 1 h. The reaction mixture was passed through a column and the product was eluted at 5% EtOAc in hexanes. The residue obtained upon evaporation of the product containing fractions was then triturated with petroleum ether to get 1.3 g of the title compound. LCMS analysis indicated the presence of a mixture of boronic acid (86%) and boronate (12%).

| | | Mass spectrum and ¹H | |
|---|---|---|---|
| Compound | Structure | NMR | SM |
| Intermediate 359 | 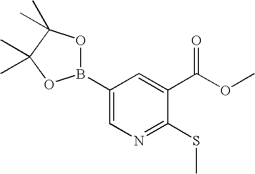 Methyl 2- (methylsulfanyl)-5- (4,4,5,5-tetramethyl- 1,3,2-dioxaborolan-2- yl)pyridine-3- carboxylate | MS(ES): 310 (M + 1) for C₁₄H₂₀BNO₄S (12% as Boronic ester) and 228 (M + 1) for C₈H₁₀BNO₄S (86% as Boronic acid). 400 MHz, DMSO-d₆: δ 1.31 (s, 12H), 2.47 (s, 3H), 3.86 (s,3H), 8.37 (s, 1H), 8.77 (s, 1H). | Intermediate 358 Methyl 5- bromo-2- (methylthio) nicotinate |

### Intermediate 360: ethyl 5-bromo-1-ethyl-2-oxo-1,2-dihydropyridine-3-carboxylate

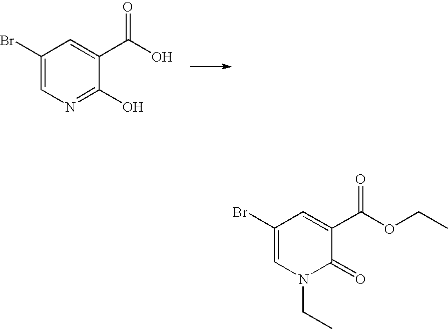

To a suspension of cesium carbonate (12.3 mmol, 4 g) in dry ethanol (20 mL), 5-bromo-2-hydroxypyridine-3-carboxylic acid (9.2 mmol, 2 g) and iodoethane (24.3 mmol, 3.8 g) were added and heated to 80° C. for 3 h in a sealed tube. The mixture was diluted with methanol and filtered through a celite bed. The filtrate was concentrated to yield 2.2 g of the title compound.

| | | Mass spectrum and ¹H | |
|---|---|---|---|
| Compound | Structure | NMR | SM |
| Intermediate 360 | 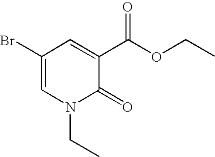 ethyl 5-bromo-1- ethyl-2-oxo-1,2- dihydropyridine-3- carboxylate | MS(ES): 274 (M) and 276 (M + 2) for C₁₀H₁₂BrNO₃. 300 MHz, CDCl₃: δ 1.32- 1.40 (m, 6H), 4.02 (q, J = 7.17 Hz, 2H), 4.36 (q, J = 7.14 Hz, 2H), 7.64 (q, J = 2.88 Hz, 1H), 8.12 (d, J = 2.91 Hz, 1H). | 5-bromo-2- hydroxy- pyridine-3- carboxylic acid |

### Intermediate 361: ethyl1-ethyl-2-oxo-5-(4,4,5,5-tetramethyl-1,3,2-dioxaborolan-2-yl)-1,2-dihydropyridine-3-carboxylate

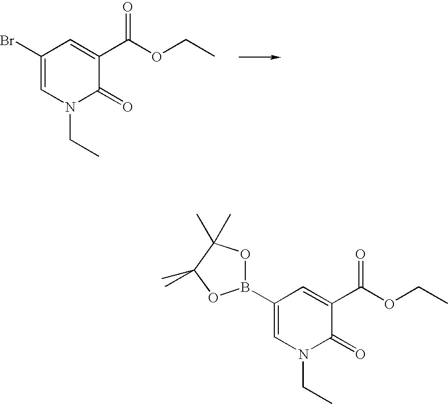

Ethyl 5-bromo-1-ethyl-2-oxo-1,2-dihydropyridine-3-carboxylate (Intermediate 360, 1.82 mmol, 0.5 g), bis(pinacolato)diboron (2.1 mmol, 0.56 g), [1,1′-bis(diphenylphosphino)ferrocene]dichloropalladium(II)complex with CH₂Cl₂ (0.27 mmol, 0.22 g) and potassium acetate (5.4 mmol, 0.53 g) were suspended in dry dioxane (10 mL) and degassed with nitrogen for 10 min. The reaction was then heated to 100° C. for 1 h. The reaction mixture was diluted with DCM and filtered through a celite bed and concentrated to give the crude title compound which was used in the next step without further purification. HPLC-MS analysis indicated the presence of a mixture of boronic acid (39%) and boronate (35%).

| | | Mass spectrum and ¹H | |
|---|---|---|---|
| Compound | Structure | NMR | SM |
| Intermediate 361 | 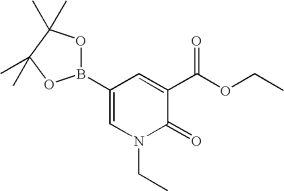 Ethyl 1-ethyl-2-oxo- 5-(4,4,5,5- tetramethyl-1,3,2- dioxaborolan-2-yl)- 1,2-dihydropyridine- 3-carboxylate | Taken to the next step as a mixture based on LCMS without further purification MS(ES): 240 (M + 1) for C₁₀H₁₄BNO₅ (39% as Boronic acid) and MS(ES): 322 (M + 1) for C₁₆H₂₄BNO₅ (35% as Boronic ester). | Intermediate 360 ethyl 5- bromo-1- ethyl-2-oxo- 1,2-dihydro- pyridine-3- carboxylate |

### Intermediate

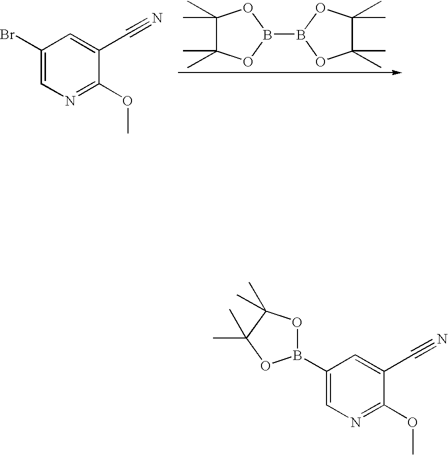

### 362: 2-methoxy-5-(4,4,5,5-tetramethyl-1,3,2-dioxaborolan-2-yl)nicotinonitrile

5-Bromo-2-methoxynicotinonitrile (0.5 g, 2.35 mmol), 4,4,4′,4′,5,5,5′,5′-octamethyl-2,2′-bi(1,3,2-dioxaborolane) (0.834 g, 3.29 mmol), 1,1′-bis(diphenylphosphino)ferrocene-palladium dichloride-dichloromethane adduct (0.575 g, 0.70 mmol), and potassium acetate (0.691 g, 7.04 mmol) were suspended in 1,4-dioxane (20 ml) and degassed with nitrogen for 10 min. The reaction was then heated at 90° C. for 2 h, diluted with dichloromethane and purified by flash chromatography (25 g silica column, 0-8% methanol in dichloromethane). Fractions were combined to obtain reddish-brown solid corresponding to title compound.

MS(ES): 260.99 (M+H) for C₁₃H₁₇BN₂O₃

¹H-NMR (400 MHz, DMSO-d6): δ ppm 1.30 (s, 12H) 4.03 (s, 3H) 8.30 (d, J=1.70 Hz, 1H) 8.62 (d, J=1.70 Hz, 1H).

### Intermediate 363: 5-bromo-N-(3,5-dimethoxy)phenyl]-4-(methylthio)pyrimidin-2-amine

The title compound was prepared using the general method described above for Intermediate 65 using 5-bromo-2-chloro-4-(methylthio)pyrimidine and 3,5-dimethoxy-aniline.

| Int | Compound | Data | SM |
|---|---|---|---|
| Intermediate 363 | 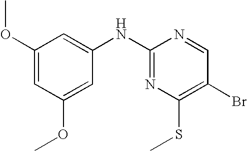 5-bromo-N-(3,5- dimethoxy)phenyl]-4- (methylthio)pyrimidin-2- amine | MS: ES+ 356 for C₁₃H₁₄BrN₃O₂S 1H NMR (300 MHz, DMSO- D6) δ ppm 2.58 (s, 3 H) 3.72 (s, 6 H) 6.16 (t, J = 2.26 Hz, 1 H) 7.00 (d, J = 2.26 Hz, 2 H) 8.31 (s, 1 H) 9.69 (s, 1 H) | 3,5- dimethoxy- aniline |

### Intermediate 364: 2-(3-chloro-4-fluorophenylamino)-4-(3-(trifluoromethyl)-1H-pyrazol-1-yl)pyrimidin-5-ylboronic acid

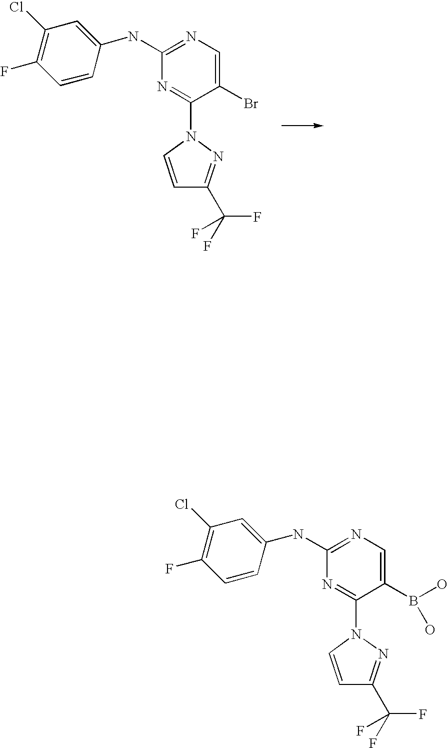

5-bromo-N-(3-chloro-4-fluorophenyl)-4-(3-(trifluoromethyl)-1H-pyrazol-1-yl)pyrimidin-2-amine Intermediate 115 (300 mg, 0.69 mmol) and triisopropyl borate (0.319 mL, 1.37 mmol) were combined in anhydrous THF (3.00 mL) and anhydrous toluene (12 mL) to give a colorless solution under argon. The mixture was cooled to −78° C., then 2.5M BuLi in Hexanes (0.550 mL, 1.37 mmol) was added dropwise over 1 hour. After 30 minutes, 1 M HCl (20 ml) was added at −78° C., then the the ice bath was removed and the mixture was allowed to warm to RT. The mixture was concentrated followed by addition of water and ethyl acetate. The water layer was extracted with ethyl acetate. The combined organic layers were washed with brine (1×50 mL) then dried over MgSO₄. The residue after filtration and evaporation was purified by silica gel chromatography using 0-10% MeOH in methylene chloride. The title compound was isolated as a white solid. (80 mg).

MS (Electrospray): 402 (MH⁺) C₁₄H₉BClF₄N₅O₂

### Intermediate 365: 5-Bromo-2-methoxy-N-(methylsulfonyl)nicotinamide

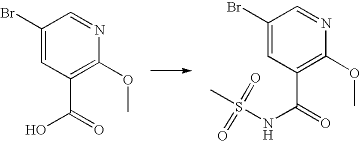

To a stirred suspension of 5-bromo-2-methoxynicotinic acid (1.15 g, 4.96 mmol) and oxalyl chloride (0.649 ml, 7.43 mmol) in methylene chloride (10 mL), under an atmosphere of nitrogen at ambient temperature, were added two drops of DMF. This mixture was allowed to stir for 2 hours. The solution was concentrated under vacuum; the residue was redissolved in methylene chloride (5 mL) and added dropwise to a stirred suspension of methanesulfonamide (0.471 g, 4.96 mmol) and pyridine (0.802 ml, 9.91 mmol) in methylene chloride (5 mL), under an atmosphere of nitrogen at ambient temperature. This mixture was stirred overnight, concentrated and purified by flash chromatography (silica gel, 0-6% methanol in methylene chloride) to yield the title compound (1.1 g). MS: ES+ 310 for C₈H₉BrN₂O₄S.

1H NMR (300 MHz, CHLOROFORM-d) δ ppm 3.41 (s, 3H) 4.16 (s, 3H) 8.43 (d, J=2.64 Hz, 1H) 8.58 (d, J=2.64 Hz, 1H) 10.05 (br. s., 1H)

### Intermediate 366: Methyl5-bromo-1-methyl-2-oxo-1,2-dihydropyridine-3-carboxylate

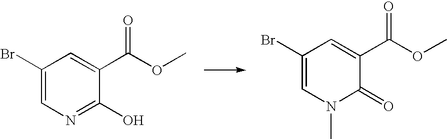

Methyl5-bromo-2-hydroxynicotinate (0.9 g, 3.88 mmol), dimethyl sulfate (1.186 mL, 12.41 mmol), and triethylamine (1.730 mL, 12.41 mmol) were dissolved in MeOH (10.5 mL) and heated in a microwave reactor at 100° C. for 30 min. The reaction was diluted with DCM, washed with water, and the organic layer was evaporated and purified by flash chromatography (silica gel, 0-12% MeOH in DCM) to afford the desired product (847 mg). MS: ES+ 247 for C₈H₈BrNO₃

1H NMR (300 MHz, DMSO-d6) d ppm 3.45 (s, 3H) 3.75 (s, 3H) 8.04 (d, J=3.01 Hz, 1H) 8.36 (d, J=2.83 Hz, 1H)

The compound in the table below was prepared using the procedure described above for Intermediate 366 using iodoethane as the alkylating agent, potassium carbonate as the base and ethanol as solvent.

| Intermediate | Compound | Mass and NMR | SM |
|---|---|---|---|
| Intermediate 367 | 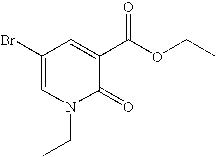 ethyl 5-bromo-1-ethyl-2-oxo- 1,2-dihydropyridine-3- carboxylate | MS: ES+ 275 for C₁₀H₁₂BrNO₃ 1H NMR (300 MHz, DMSO-d₆) d ppm 1.24 (dt, J = 10.78, 7.13 Hz,6 H) 3.94 (q, J = 7.16 Hz, 2 H) 4.21 (q, J = 7.10 Hz, 2 H) 8.01 (d, J = 2.83 Hz, 1 H) 8.35 (d, J = 2.83 Hz, 1 H) | methyl 5- bromo-2- hydroxy nicotinate |

The intermediates in the table below were prepared using the procedure for Intermediate 134 and the specified starting material.

| Intermediate | Compound | Mass and NMR | SM |
|---|---|---|---|
| Intermediate 368 | 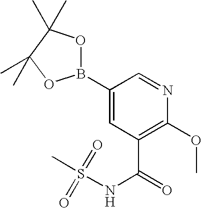 2-Methoxy-N- (methylsulfonyl)-5-(4,4,5,5- tetramethyl-1,3,2- dioxaborolan-2- yl)nicotinamide | MS: ES+ 357 for C₁₄H₂₁BN₂O₆S 1H NMR (300 MHz, CHLOROFORM-d) d ppm 1.35 (s, 12 H) 3.41 (s, 3 H) 4.19 (s, 3 H) 8.71 (d, J = 2.07 Hz, 1 H) 8.85 (d, J = 1.88 Hz, 1 H) 10.06 (s, 1 H) | Intermediate 365 |
| Intermediate 369 | 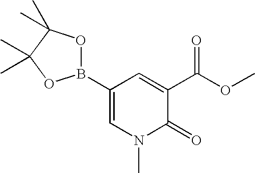 methyl 1-methyl-2-oxo-5- (4,4,5,5-tetramethyl-1,3,2- dioxaborolan-2-yl)-1,2- dihydropyridine-3- carboxylate | MS: ES+ 294 for C₁₄H₂₀BNO₅ 1H NMR (300 MHz, DMSO-d₆) d ppm 1.28 (s, 12 H) 3.50 (s, 3 H) 3.74 (s, 3 H) 8.12 (d, J = 2.26 Hz, 1 H) 8.29 (d, J = 2.26 Hz, 1 H) | Intermediate 366 |
| Intermediate 370 | 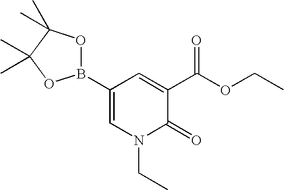 ethyl 1-ethyl-2-oxo-5- (4,4,5,5-tetramethyl-1,3,2- dioxaborolan-2-yl)-1,2- dihydropyridine-3- carboxylate | MS: ES+ 322 for C₁₆H₂₄BNO₅ 1H NMR (300 MHz, DMSO-d₆) d ppm 1.16-1.34 (m, 18 H) 4.01 (q, J = 6.91 Hz, 2 H) 4.21 (q, J = 7.03 Hz, 2 H) 8.07 (d, J = 2.07 Hz, 1 H) 8.26 (d, J = 2.26 Hz, 1 H) | Intermediate 367 |

The compounds in the table below were prepared using the procedure for Intermediate 65 and the specified starting materials.

| Intermediate | Compound | Mass and NMR | S.M. |
|---|---|---|---|
| Intermediate 371 | 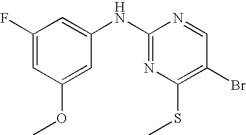 5-Bromo-N-(3-fluoro-5- methoxyphenyl)-4- (methylthio)pyrimidin-2-amine | MS: ES+ 345 for C₁₂H₁₁BrFN₃OS 1H NMR (300 MHz, DMSO-D6) δ ppm 2.59 (s, 3 H) 3.75 (s, 3 H) 6.31-6.58 (m, 1 H) 7.18 (s, 1 H) 7.23-7.38 (m, 1 H) 8.36 (s, 1 H) 9.92 (s, 1 H). | 3-Fluoro-5- methoxyaniline and 5- bromo-2-chloro-4- (methylthio)pyrimidine |
| Intermediate 372 | 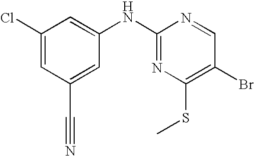 3-(5-bromo-4- (methylthio)pyrimidin-2- ylamino)-5-chlorobenzonitrile | MS: ES+ 356 for C₁₂H₈BrClN₄S 1H NMR (300 MHz, DMSO-D6) δ ppm 2.60 (s, 3 H) 7.48- 7.68 (m, 1 H) 8.08- 8.14 (m, 1 H) 8.19 (t, J = 1.98 Hz, 1 H) 8.43 (s, 1 H) 10.29 (s, 1 H). | 5-bromo-2-chloro-4- (methylthio)pyrimidine and 3-amino-5- chlorobenzonitrile |

The compounds in the table below were prepared using the procedure for Intermediate 69 and the specified starting material.

| Intermediate | Compound | Mass and NMR | S.M |
|---|---|---|---|
| Intermediate 373 | 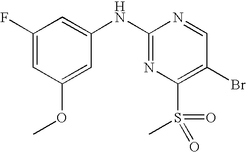 5-bromo-N-(3-fluoro-5- methoxyphenyl)-4- (methylsulfonyl)pyrimidin-2- amine | MS: ES+ 377 for C₁₂H₁₁BrFN₃O₃S 1H NMR (300 MHz, DMSO-D6) δ ppm 3.47 (s, 3 H) 3.76 (s, 3 H) 6.39-6.70 (m, 1 H) 7.08-7.36 (m, 2 H) 8.95 (s, 1 H) 10.48 (s,1 H) | Intermediate 371 |
| Intermediate 374 | 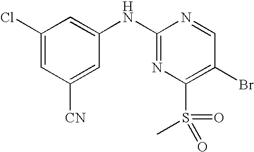 3-(5-bromo-4- (methylsulfonyl)pyrimidin-2- ylamino)-5-chlorobenzonitrile | MS: ES+ 388 for C₁₂H₈BrClN₄O₂S 1H NMR (300 MHz, DMSO-d₆) d ppm 3.36 (s, 3 H) 7.52- 7.55 (m, 1 H) 7.97 (ddd, J = 7.77, 2.07 1.84 Hz, 2 H) 8.90 (s, 1 H) 10.68 (s, 1 H) | Intermediate 372 |

The compounds in the table below were prepared using the procedure for Intermediate 112 and the specified starting materials.

| Intermediate | Compound | Mass and NMR | S.M. |
|---|---|---|---|
| Intermediate 375 | 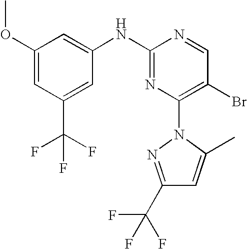 5-bromo-N-(3-methoxy-5- (trifluoromethyl)phenyl)-4-(5- methyl-3-(trifluoromethyl)-1H- pyrazol-1-yl)pyrimidin-2-amine | MS: ES+ 497 for C₁₇H₁₂BrF₆N₅O 1H NMR (300 MHz, DMSO-D6) d ppm 2.41 (s, 3 H) 3.81 (s, 3 H) 6.76-7.05 (m, 2 H) 7.60 (s, 1 H) 7.69 (s, 1 H) 9.01 (s, 1 H) 10.54 (s, 1 H) | Intermediate 70 and 3-trifluoromethyl-5- methylpyrazole |
| Intermediate 376 | 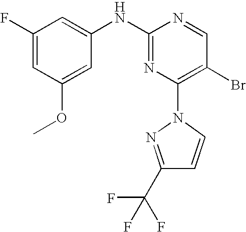 5-bromo-N-(3-fluoro-5- methoxyphenyl)-4-(3- (trifluoromethyl)-1H-pyrazol-1- yl)pyrimidin-2-amine | MS: ES+ 433 for C₁₅H₁₀BrF₄N₅O 1H NMR (300 MHz, DMSO-D6) δ ppm 3.76 (s, 3 H) 6.41- 6.69 (m, 1 H) 7.15 (d, J = 2.83 Hz, 1 H) 7.18- 7.46 (m, 2 H) 8.64 (d, J = 1.51 Hz, 1 H) 8.94 (s, 1 H) 10.40 (s, 1 H) | Intermediate 373 and 3-trifluoromethyl pyrazole |
| Intermediate 377 | 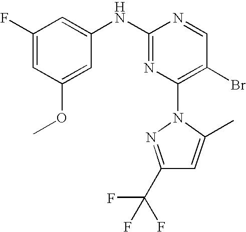 5-bromo-N-(3-fluoro-5- methoxyphenyl)-4-(5-methyl-3- (trifluoromethyl)-1H-pyrazol-1- yl)pyrimidin-2-amine | MS: ES+ 447 for C₁₆H₁₂BrF₄N₅O 1H NMR (300 MHz, DMSO-d₆): d ppm 2.40 (s, 3 H) 3.74 (s, 3 H) 6.50 (dt, J = 10.97, 2.33 Hz, 1 H) 6.85 (s, 1 H) 7.12- 7.16 (m, 1 H) 7.20 (dt, J = 11.49, 1.98 Hz, 1 H) 8.98 (s, 1 H) 10.42 (s, 1 H) | Intermediate 373 and 3- trifluoromethyl-5- methylpyrazole |
| Intermediate 378 | 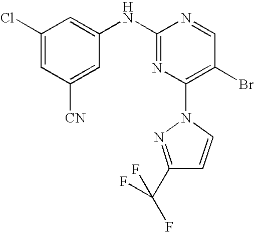 3-(5-bromo-4-(3- (trifluoromethyl)-1H-pyrazol-1- yl)pyrimidin-2-ylamino)-5- chlorobenzonitrile | MS: ES+ 444 for C₁₅H₇BrClF₃N₆ 1H NMR (300 MHz, DMSO-d₆) d ppm 7.17 (d, 1 H) 7.62- 7.65 (m, 1 H) 8.13 (ddd, J = 8.90, 2.07, 1.84 Hz, 2 H) 8.66 (dd, J = 2.73, 1.04 Hz, 1 H) 9.01 (s, 1 H) 10.73 (s, 1 H) | Intermediate 374 and 3-trifluoromethyl pyrazole |

### Intermediate 379: 5-(2-(3-chloro-4-fluorophenylamino)-4-(3-(dimethylamino)propylamino)pyrimidin-5-yl)nicotinohydrazide

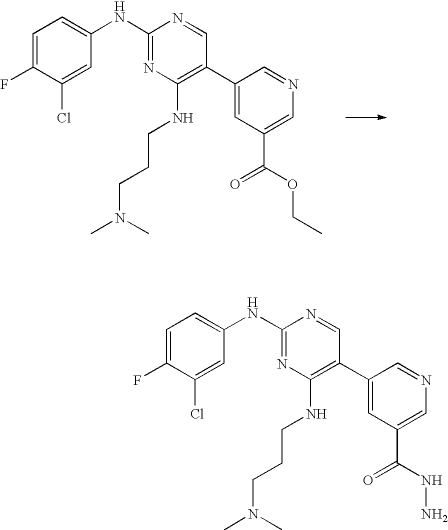

A solution of Example 5 (100 mg, 0.21 mmol) and hydrazine hydrate (0.015 mL, 0.32 mmol) in 1,4-dioxane (1 mL) was stirred and heated to 115 degrees for 60 minutes. Solvent was removed under reduced pressure, affording the title compound (83 mg).

MS: ES+ 459 for C₂₁H₂₄ClFN₈O.

1H NMR (300 MHz, DMSO-d6) d ppm 1.70 (quin, J=6.59 Hz, 2H) 2.02 (s, 6H) 2.33 (t, J=6.50 Hz, 2H) 3.38-3.50 (m, 2H) 7.30 (t, J=9.14 Hz, 1H) 7.41 (t, J=4.99 Hz, 1H) 7.63 (ddd, J=9.14, 4.24, 2.64 Hz, 1H) 7.86 (s, 1H) 8.13 (t, J=2.07 Hz, 1H) 8.26 (dd, J=6.97, 2.64 Hz, 1H) 8.68 (d, J=2.07 Hz, 1H) 8.93 (d, J=1.88 Hz, 1H) 9.48 (s, 1H) 9.99 (br. s., 1H)

### Intermediate 380: 2-(3-chloro-4-fluorophenylamino)-4-(3-methoxypropylamino)pyrimidine-5-carboxamide

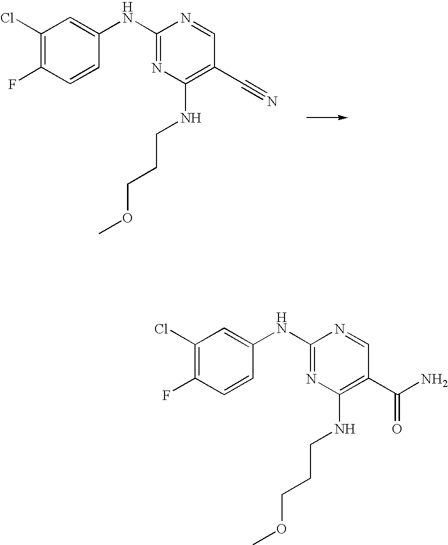

Intermediate 127 (120 mg, 0.35 mmol) was suspended in methanol (0.5 mL) and water (0.5 mL) and stirred under ambient conditions. To this mixture was added aqueous sodium hydroxide (50 wt %, 84 mg, 1.05 mmol). Upon warming to 50 degrees a small amount of dioxane was added to aid in solubility. After 90 minutes the mixture was removed from heating then water was added to precipitate a solid; this was collected and washed with water to give the title compound (87 mg). MS: ES+ 354 for C₁₅H₁₇ClFN₅O₂.

1H NMR (300 MHz, DMSO-d₆) δ ppm 1.81 (quin, J=6.50 Hz, 2H) 3.22 (s, 3H) 7.18 (br. s., 1H) 7.30 (t, J=9.14 Hz, 1H) 7.54-7.66 (m, 1H) 7.80 (br. s., 1H) 8.20 (dd, J=6.78, 2.45 Hz, 1H) 8.52 (s, 1H) 9.20 (t, J=5.37 Hz, 1H) 9.69 (s, 1H).

The following intermediates were prepared using the general method described above for Intermediate 72 using 5- bromo-2-[N-(3-chloro-4-fluorophenyl)]-4-(methyl sulfonyl)pyrimidin-2-amine (Intermediate 69) and the starting material (SM) indicated.

| Int | Compound | Data | SM |
|---|---|---|---|
| Intermediate 381 | 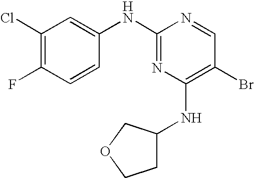 5-bromo-N²-(3- chloro-4- fluorophenyl)-N⁴- (tetrahydrofuran-3- yl)pyrimidin-2,4- diamine | MS(ES): 387 (M) 389 (M + 3) for C₁₄H₁₃BrClFN₄O 1H NMR (300 MHz, DMSO-d₆) δ ppm 1.90-2.13 (m, 1 H) 2.13- 2.33 (m, 1 H) 3.57-3.81 (m, 2 H) 3.81-4.05 (m, 2 H) 4.42-4.72 (m, 1 H) 6.86 (d, J = 6.22 Hz, 1 H) 7.31 (t, J = 9.14 Hz, 1 H) 7.52 (ddd, J = 9.14, 4.24, 2.64 Hz, 1 H) 8.03- 8.21 (m, 2 H) 9.51 (s, 1 H) | 3- Aminotetra- hydrofuran |
| Intermediate 382 | 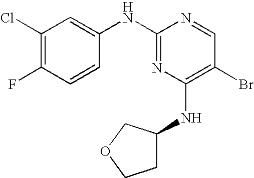 (S)-5-bromo-N²-(3- chloro-4- fluorophenyl)-N⁴- (tetrahydrofuran-3- yl)pyrimidin-2,4- diamine | MS(ES): 387 (M) 389 (M + 3) for C₁₄H₁₃BrClFN₄O 1H NMR (300 MHz, DMSO-d₆) δ ppm 1.90-2.13 (m, 1 H) 2.13- 2.33 (m, 1 H) 3.57-3.81 (m, 2 H) 3.81-4.05 (m, 2 H) 4.42-4.72 (m, 1 H) 6.86 (d, J = 6.22 Hz, 1 H) 7.31 (t, J = 9.14 Hz, 1 H) 7.52 (ddd, J = 9.14, 4.24, 2.64 Hz, 1 H) 8.03- 8.21 (m, 2 H) 9.51 (s, 1 H) | S(−)-3- Aminotetra- hydrofuran |

### Intermediate 383: (4-(5-bromo-2-(3-chloro-4-fluorophenylamino)pyrimidin-4-yl)morpholin-2-yl)methanol

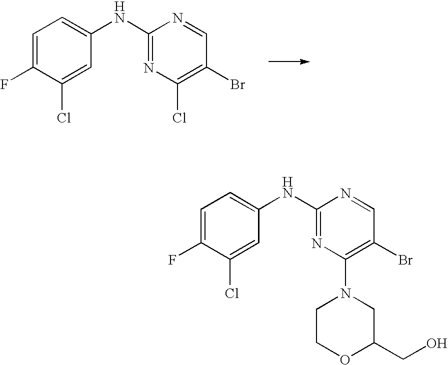

To 600 mg of 5-bromo-4-chloro-N-(3-chloro-4-fluorophenyl)pyrimidin-2-amine (Intermediate 63, 1.78 mmol) in 1,4-Dioxane (8 mL), was added triethylamine (0.27 mL, 1.96 mmol) and 2-hydroxymethylmorpholine (1.78 mmol, 209 mg) under inert atmosphere. The reaction mixture was stirred at room temperature for 2 days. The reaction mixture was diluted with ethyl acetate and MeOH and adsorbed on silica gel. The mixture was purified by column chromatography using 0-10% MeOH in DCM to obtain (4-(5-bromo-2-(3-chloro-4-fluorophenylamino)pyrimidin-4-yl)morpholin-2-yl)methanol (473 mg). MS(ES): 417 (M) and 419 (M+2) for C₁₅H₁₅BrClFN₄O₂.

¹H NMR (300 MHz, DMSO-D6) δ ppm 2.71-2.94 (m, 1H) 2.94-3.14 (m, 1H) 3.33-3.76 (m, 4 H) 3.92 (d, J=11.11 Hz, 1H) 4.09 (d, J=12.81 Hz, 1H) 4.22 (d, J=13.00 Hz, 1H) 4.82 (t, J=5.27 Hz, 1H) 7.30 (t, J=9.14 Hz, 1H) 7.43-7.70 (m, 1H) 7.89-8.14 (m, 1H) 8.26 (s, 1H) 9.70 (s, 1H).

The following compound was prepared using the general method described for Intermediate 383 and the starting materials (SM) indicated.

| Int | Compound | Data | SM |
|---|---|---|---|
| Intermediate 384 | 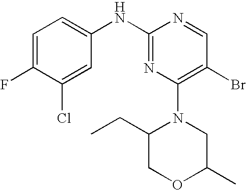 5-bromo-N-(3-chloro- 4-fluorophenyl)-4-(5- ethyl-2- methylmorpholin)- pyrimidin-2-amine | MS(ES): 429 (M) and 431 (M + 2) for C₁₇H₁₉BrClFN₄O | 5-bromo-4- chloro-N-(3- chloro-4- fluorophenyl) pyrimidin-2- amine Intermediate 63 and 5-ethyl-2- methyl- morpholine |

The following compound was prepared using the general method described for Intermediate 131 and the starting material (SM) indicated.

| Int | Compound | Data | SM |
|---|---|---|---|
| Intermediate 385 | 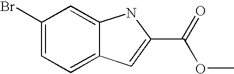 Methyl 6-bromo- 1H-indole-2- carboxylate | ¹H NMR (300 MHz, DMSO-D6) δ ppm 3.87 (s, 3 H) 7.09-7.28 (m, 2 H) 7.49-7.80 (m, 2 H) 12.07 (s, 1 H) | 6- Bromoindole- 2-carboxylic acid |

### Intermediate 386: 1-tert-butyl 2-methyl 6-bromo-1H-indole-1,2-dicarboxylate

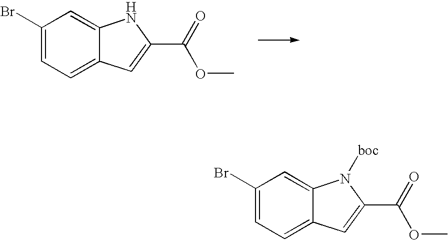

To 1 g of methyl 6-bromo-1H-indole-2-carboxylate (Intermediate 385, 3.94 mmol) in THF (20 mL) was added di-t-Butyl dicarbonate (1.074 g, 4.92 mmol) and treated with 4-dimethylaminopyridine (48 mg, 0.39 mmol). The mixture was stirred at room temperature under nitrogen for 4 days. The mixture was concentrated at reduced pressure and the residue was adsorbed on silica gel and purified by column chromatography with 0-25% EtOAc in hexanes to afford 1-tert-butyl 2-methyl6-bromo-1H-indole-1,2-dicarboxylate (1.33 g).

¹H NMR (300 MHz, DMSO-D6) δ ppm 1.55 (s, 9 H) 3.86 (s, 3H) 7.29 (s, 1H) 7.49 (dd, J=8.48, 1.70 Hz, 1H) 7.69 (d, J=8.48 Hz, 1H) 8.13 (s, 1H).

The following compound was prepared using the general method described above for Intermediate 386 using the starting material (SM) indicated.

| Int | Compound | Data | SM |
|---|---|---|---|
| Intermediate 387 | 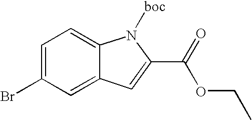 1-tert-butyl 2-ethyl-5- bromo-1H-indole- 1,2-dicarboxylate | ¹H NMR (300 MHz, DMSO-D6) δ ppm 1.31 (t, J = 7.06 Hz, 3 H) 1.56 (s, 9 H) 4.32 (q, J = 7.16 Hz, 2 H) 7.23 (s, 1 H) 7.60 (dd, J = 8.85, 2.07 Hz, 1 H) 7.82-8.06 (m, 2 H) | Ethyl 5- bromo-1H- indole-2- carboxylate |

The following compounds were prepared using the general method described above for Intermediate 133 using Intermediate 132 and the starting material (SM) indicated.

| Int | Compound | Data | SM |
|---|---|---|---|
| Intermediate 388 | 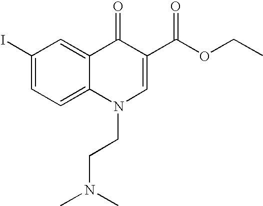 ethyl 1-(2- (dimethylamino)ethyl)- 6-iodo-4-oxo-1,4- dihydroquinoline-3- carboxylate | MS(ES): 415 (M + 1) for C₁₆H₁₉IN₂O₃ ¹H NMR (300 MHz, DMSO-d₆) δ ppm 1.28 (t, 3 H) 2.17 (s, 6 H) 2.57 (t, J = 5.84 Hz, 2 H) 4.22 (q, J = 7.16 Hz, 2 H) 4.44 (t, J = 5.93 Hz, 2 H) 7.65 (d, J = 9.04 Hz,1 H) 8.06 (dd, J = 8.85, 2.26 Hz, 1 H) 8.50 (d, J = 2.07 Hz, 1 H) 8.63 (s, 1 H) | N1,N1- dimethylethane- 1,2-diamine |
| Intermediate 389 | 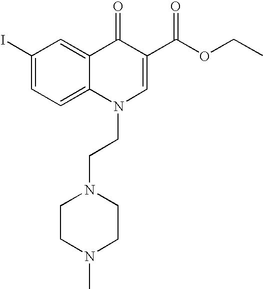 Ethyl 6-iodo-1-(2-(4- methylpiperazin-1- yl)ethyl)-4-oxo-1,4- dihydroquinoline-3- carboxylate | MS(ES): 470 (M + 1) for C₁₉H₂₄IN₃O₃ ¹H NMR (300 MHz, DMSO-d₆) δ ppm 1.29 (t, 3 H) 2.11 (s, 3 H) 2.14-2.49 (m, 8 H) 2.59 (t, J = 5.56 Hz, 2 H) 4.23 (q, J = 7.03 Hz, 2 H) 4.43 (t, J = 5.65 Hz, 2 H) 7.67 (d, J = 8.85 Hz, 1 H) 8.05 (dd, J = 8.95, 2.17 Hz, 1 H) 8.50 (d, J = 2.26 Hz, 1 H) 8.59 (s, 1 H) | 2-(4- Methylpiperazin- 1- yl)ethanamine |

### Intermediate 390: 3-bromo-5-(methylsulfonyl)pyridine

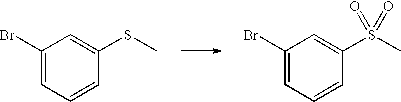

A solution of 3-bromo-5-(methylthio)pyridine (2.17 g, 10.63 mmol) in DCM (40 mL) was cooled to 0° C. The reaction was then treated with m-Chloroperbenzoic acid (4.89 g, 21.27 mmol) and allowed to stir at 0° C. for 30 min (reaction became a suspension after mcpba addition) before it was allowed to warm up to room temperature for 1 hr. The reaction mixture was diluted with EtOAc, basified with sodium carbonate, and the layers were separated. The organic layer was washed with brine and dried over MgSO₄. The solvent was removed at reduced pressure and the residue was adsorbed on silica and purified by column chromatography with 50-100% EtOAc in hexanes to afford 3-bromo-5-(methylsulfonyl)pyridine (1.97 g). MS(ES): 236 (M) and 238 (M+2) for C₆H₆BrNO₂S.

¹H NMR (300 MHz, DMSO-D6) δ ppm 3.39 (s, 3H) 8.57 (t, J=2.07 Hz, 1H) 9.04 (d, J=1.88

Hz, 1H) 9.07 (d, J=2.07 Hz, 1H).

### Intermediate 391: tert-butyl2-(3-bromophenylthio)acetate

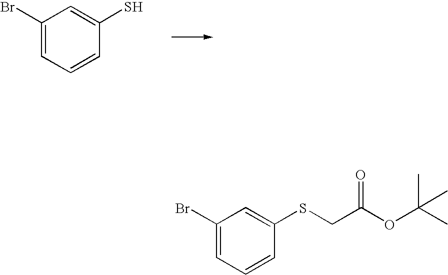

A solution of 3-bromobenzenethiol (0.788 mL, 6.66 mmol) in DMF (12 mL) was treated with tert-butyl2-bromoacetate (1.034 mL, 7.00 mmol) and potassium carbonate (1.842 g, 13.33 mmol). The reaction was stirred at room temperature under nitrogen overnight. The reaction was diluted with EtOAc/H₂O and the layers were separated. The organic phase was washed with brine and dried over sodium sulfate. The solvent was removed at reduced pressure and the residue was adsorbed on silica and purified by column chromatography with 0-20% EtOAc in hexanes to afford tert-butyl2-(3-bromophenylthio)acetate (1.6 g).

¹H NMR (300 MHz, DMSO-d₆) δ ppm 1.35 (s, 9 H) 3.82 (s, 2H) 7.15-7.47 (m, 3H) 7.54 (t, J=1.79 Hz, 1H).

The following compound was prepared using the general method described above for Intermediate 390 using mcpba and the starting material (SM) indicated.

| Int | Compound | Data | SM |
|---|---|---|---|
| Intermediate 392 | 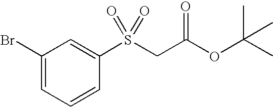 tert-butyl 2-(3- bromophenylsulfonyl)- acetate | ¹H NMR (300 MHz, CHLOROFORM-d) δ ppm 1.40 (s, 9 H) 4.05 (s, 2 H) 7.47 (t, J = 8..01 Hz, 1 H) 7.74-7.97 (m, 2 H) 8.10 (t, J = 1.79 Hz, 1 H) | tert-butyl 2-(3- bromophenyl- thio)acetate Intermediate 391 |

### Intermediate 393: Ethyl2-(3-bromophenylamino)-2-oxoacetate

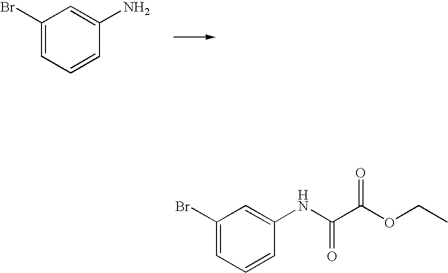

3-Bromoaniline (0.759 mL, 6.98 mmol) was dissolved in THF (20 mL), treated with triethylamine (0.972 mL, 6.98 mmol), and cooled to 0° C. The solution was then treated with ethyl 2-chloro-2-oxoacetate (0.779 mL, 6.98 mmol) and allowed to slowly warm up to room temperature and stir overnight under nitrogen. The reaction was diluted with EtOAc/H₂O and the layers were separated. The organic phase was washed with brine and dried over sodium sulfate. The solvent was removed at reduced pressure, the residue was adsorbed on silica, and purified by column chromatography with 0-60% EtOAc in hexanes to afford Ethyl2-(3-bromophenylamino)-2-oxoacetate (1.78 g).

MS(ES): 272 (M) and 274 (M+2) for C₁₀H₁₀BrNO₃.

¹H NMR (300 MHz, DMSO-d₆) δ ppm 1.32 (t, J=7.06 Hz, 3H) 4.31 (q, J=7.16 Hz, 2H) 7.19-7.44 (m, 2H) 7.74 (dt, J=6.45, 2.33 Hz, 1H) 7.90-8.17 (m, 1H) 10.92 (s, 1H)

### Intermediate 394: 3-Bromo-N-(ethylcarbamoyl)benzenesulfonamide

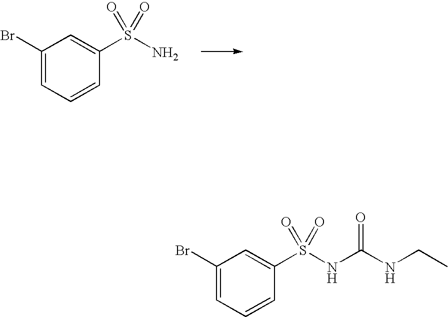

A solution of 3-bromobenzenesulfonamide (400 mg, 1.69 mmol) in acetone (4.20 mL) was treated with a solution of potassium hydroxide (95 mg, 1.69 mmol) in water (0.6 mL). The reaction was stirred at room temperature for 15 min at which time the solvent was removed at reduced pressure. The residue was re-dissolved in DMF (4.20 mL), treated with ethyl isocyanate (0.266 mL, 3.39 mmol), and stirred overnight. The solvent was removed at reduced pressure and the residue was basified with 2 mL of 1 N NaOH, diluted with water, and then acidified with concentrated HCl. The solid formed was then filtered and dried to afford the desired product (350 mg).

¹H NMR (300 MHz, DMSO-d₆) δ ppm 0.95 (t, 3H) 2.81-3.13 (m, 2H) 6.61 (t, J=5.37 Hz, 1H) 7.58 (t, J=7.91 Hz, 1H) 7.90 (dt, J=8.15, 1.77 Hz, 2H) 8.03 (t, J=1.79 Hz, 1H) 10.73 (s, 1H)

### Intermediate 395: 5-bromo-N-ethylpyridine-3-sulfonamide

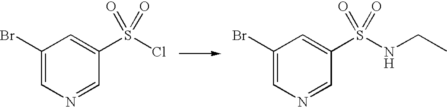

5-bromopyridine-3-sulfonyl chloride hydrochloride (1 g, 3.41 mmol) and ethanamine hydrochloride (0.306 g, 3.75 mmol) were treated with pyridine (2.76 ml, 34.13 mmol) and stirred at room temperature under nitrogen for 2 hrs. The reaction mixture was then diluted with EtOAc, washed with water, and dried over sodium sulfate. The solvent was removed at reduced pressure, the residue was adsorbed on silica, and purified on column chromatography with 10-80% EtOAc in hexanes to afford 5-bromo-N-ethylpyridine-3-sulfonamide (466 mg).

MS(ES): 265 (M) and 267 (M+2) for C₇H₉BrN₂O₂S.

¹H NMR (300 MHz, DMSO-d₆) δ ppm 0.99 (t, J=7.16 Hz, 3H) 2.78-2.96 (m, 2H) 7.93 (br. s., 1H) 8.37 (t, J=2.07 Hz, 1H) 8.92 (d, J=1.88 Hz, 1H) 9.00 (d, J=2.07 Hz, 1H).

### Intermediate 396: 3-Bromo-N-(methylsulfonyl)benzamide

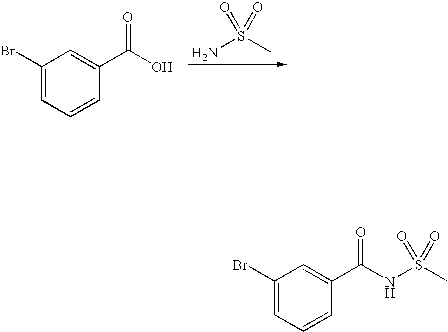

3-Bromobenzoic acid (1 g, 4.97 mmol), methanesulfonamide (0.521 g, 5.47 mmol), 1-(3-dimethylaminopropyl)-3-ethylcarbodiimide hydrochloride (1.144 g, 5.97 mmol), and 4-dimethylaminopyridine (0.304 g, 2.49 mmol) were dissolved in THF (10 mL) and stirred at room temperature overnight. The residue was diluted with EtOAc, washed with water and brine, and dried over sodium sulfate. The solvent was removed at reduced pressure, the residue was adsorbed on silica, and purified by column chromatography with 20-100% EtOAc in hexanes to afford 3-bromo-N-(methylsulfonyl)benzamide (350 mg).

¹H NMR (300 MHz, CHLOROFORM-d) δ ppm 3.46 (s, 3H) 7.40 (t, J=7.91 Hz, 1H) 7.68-7.86 (m, 2H) 8.03 (t, J=1.70 Hz, 1H) 8.67 (br. s., 1H).

### Intermediate 397: 5-bromo-1-(2-morpholinoethyl)-2-oxo-1,2-dihydropyridine-3-carboxylic acid

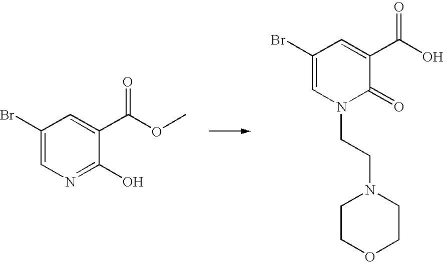

A suspension of methyl 5-bromo-2-hydroxynicotinate (1 g, 4.31 mmol), 4-(2-chloroethyl)morpholine hydrochloride (0.802 g, 4.31 mmol), and potassium carbonate (1.787 g, 12.93 mmol) in MeOH (20 mL) was refluxed overnight. The solvent was removed at reduced pressure and the residue was dissolved in H₂O and neutralized with 1N HCl. The solid formed was filtered (unreacted starting hydroxynicotinate) and the desired product remained in the aqueous layer, which was then concentrated. The residue was re-dissolved in DCM/MeOH, adsorbed on silica, and purified by column chromatography with 0-20% MeOH in DCM to afford bromo-1-(2-morpholinoethyl)-2-oxo-1,2-dihydropyridine-3-carboxylic acid (750 mg)

¹H NMR (300 MHz, DMSO-d₆) δ ppm 2.31-2.47 (m, 4 H) 2.64 (t, J=6.12 Hz, 2H) 3.43-3.69 (m, 4 H) 4.18 (t, J=6.03 Hz, 2H) 8.36 (d, J=2.83 Hz, 1H) 8.50 (d, J=2.83 Hz, 1H) 14.30 (br. s., 1H)

### Intermediate 398: methyl5-bromo-1-(2-morpholinoethyl)-2-oxo-1,2-dihydropyridine-3-carboxylate

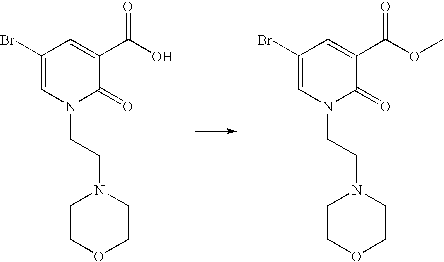

A suspension of 5-bromo-1-(2-morpholinoethyl)-2-oxo-1,2-dihydropyridine-3-carboxylic acid (Intermediate 397, 750 mg, 2.26 mmol) in methanol (9162 μl, 226.47 mmol) was treated with sulfuric acid (483 μl, 9.06 mmol). The reaction was refluxed for 4 hrs at which time the solvent was removed at reduced pressure. The residue was diluted with EtOAc and carefully neutralized with a solution of sodium carbonate. The layers were then separated and the organic was washed with brine and dried over sodium sulfate. The solvent was removed at reduced pressure and the residue was adsorbed on silica and purified by column chromatography with 0-15% MeOH in DCM to yield methyl5-bromo-1-(2-morpholinoethyl)-2-oxo-1,2-dihydropyridine-3-carboxylate (664 mg).

¹H NMR (300 MHz, DMSO-d₆) δ ppm 2.33-2.46 (m, 4 H) 2.54 (t, J=6.22 Hz, 2H) 3.39-3.60 (m, 4 H) 3.74 (s, 3H) 4.02 (t, J=6.22 Hz, 2H) 8.05 (d, J=3.01 Hz, 1H) 8.26 (d, J=3.01 Hz, 1H)

### Intermediate 399: Methyl5-bromo-2-(2-morpholinoethylamino)nicotinate

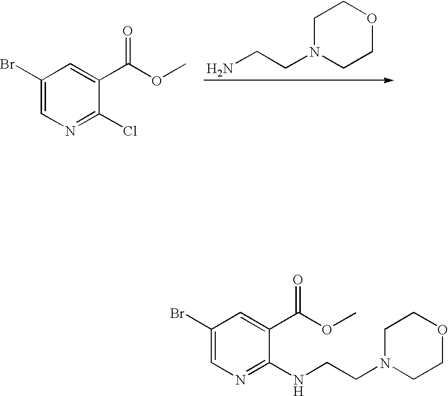

A suspension of methyl5-bromo-2-chloronicotinate (0.5 g, 2.00 mmol) and 2-morpholinoethanamine (0.390 mL, 2.99 mmol) in EtOH (3 mL) was heated in a microwave reactor at 140° C. for 45 min. The mixture was concentrated at reduced pressure. The residue was then diluted with water, treated with sodium bicarbonate, and extracted with EtOAc. The organic layer was washed with brine and dried over sodium sulfate. The solvent was removed at reduced pressure and the residue was adsorbed on silica and purified by column chromatography with 0-16% MeOH in DCM to afford methyl 5-bromo-2-(2-morpholinoethylamino)nicotinate (582 mg).

¹H NMR (300 MHz, DMSO-d₆) δ ppm 2.29-2.46 (m, 4 H) 2.51-2.60 (m, 2H) 3.43-3.67 (m, 6 H) 3.84 (s, 3H) 8.05-8.26 (m, 2H) 8.37 (d, J=2.45 Hz, 1H)

The following compound was prepared using the general method described above for Intermediate 399 using methyl5-bromo-2-chloronicotinate and the starting material (SM) indicated.

| Int | Compound | Data | SM |
|---|---|---|---|
| Intermediate 400 | 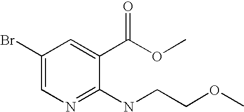 Methyl 5-bromo-2-(2- methoxyethylamino)- nicotinate | MS(ES): 289 (M) and 291 (M + 2) for C₁₀H₁₃BrN₂O₃ ¹H NMR (300 MHz, DMSO- d₆) δ ppm 3.29 (s, 3 H) 3.43- 3.55 (m, 2 H) 3.60 (q, J = 5.15 Hz, 2 H) 3.83 (s, 3 H) 8.07 (t, J = 4.90 Hz, 1 H) 8.15 (d, J = 2.64 Hz, 1 H) 8.38 (d, J = 2.45 Hz, 1 H) | 2- methoxyethan amine |

The following compounds were prepared using the general method described above for Intermediate 367 using methyl5-bromo-2-hydroxynicotinate and the starting material (SM) indicated.

| Int | Compound | Data | SM |
|---|---|---|---|
| Intermediate 401 | 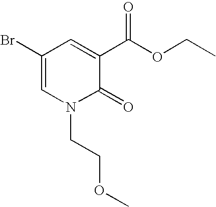 Ethyl 5-bromo-1-(2- methoxyethyl)-2-oxo- 1,2-dihydropyridine- 3-carboxylate | MS(ES): 304 (M) and 306 (M + 2) for C₁₁H₁₄BrNO₄ ¹H NMR (300 MHz, DMSO- d₆) δ ppm 1.26 (t, 3 H) 3.25 (s, 3 H) 3.57 (t, J = 5.27 Hz, 2 H) 4.10 (t, J = 5.37 Hz, 2 H) 4.21 (q, J = 7.03 Hz, 2 H) 8.03 (d, J = 3.01 Hz, 1 H) 8.21 (d, J = 3.01 Hz, 1 H) | 2-Bromoethyl methylether |
| Intermediate 402 | 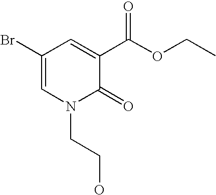 Ethyl 5-bromo-1-(2- hydroxyethyl)-2-oxo- 1,2-dihydropyridine- 3-carboxylate | MS(ES): 290 (M) and 292 (M + 2) for C₁₀H₁₂BrNO₄ ¹H NMR (300 MHz, DMSO- d₆) δ ppm 1.26 (t, 3 H) 3.62 (q, J = 5.46 Hz, 2 H) 3.98 (t, J = 5.27 Hz, 2 H) 4.21 (q, J = 7.10 Hz, 2 H) 4.91 (t, J = 5.46 Hz, 1 H) 8.03 (d, J = 3.01 Hz, 1 H) 8.16 (d, J = 2.83 Hz, 1 H) | 2-Bromoethanol |
| Intermediate 403 | 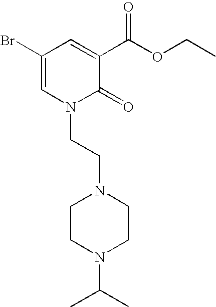 Methyl 5-bromo-1- (2-(4- isopropylpiperazin-1- yl)ethyl)-2-oxo-1,2- dihydropyridine-3- carboxylate | MS(ES): 386 (M) and 388 (M + 2) for C₁₆H₂₄BrN₃O₃ ¹H NMR (300 MHz, CHLOROFORM-d) δ ppm 1.07 (br. s., 6 H) 2.35-2.89 (m, 11 H) 3.92 (s, 3 H) 4.05 (t, J = 5.93 Hz, 2 H) 7.69 (br. s., 1 H) 8.19 (d, J = 2.83 Hz, 1 H) | 1-(2- Chloroethyl)-4- isopropylpiper- azine dihydrochloride |

The following compound was prepared using the general method described above for Intermediate 366 using methyl5-bromo-2-hydroxynicotinate and the starting material (SM) indicated.

| Int | Compound | Data | SM |
|---|---|---|---|
| Intermediate 404 | 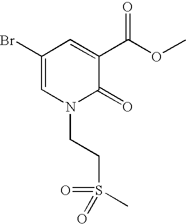 Methyl 5-bromo-1-(2- (methylsulfonyl)ethyl)-2-oxo-1,2- dihydropyridine-3-carboxylate | MS(ES): 338 (M) and 340 (M + 2) for C₁₀H₁₂BrNO₅S ¹H NMR (300 MHz, DMSO- d₆) δ ppm 3.07 (s, 3 H) 3.57 (t, J = 6.88 Hz, 2 H) 3.76 (s, 3 H) 4.33 (t, J = 6.88 Hz, 2 H) 8.08 (d, J = 3.01 Hz, 1 H) 8.36 (d, J = 3.01 Hz, 1 H) | 1-Bromo-2- (methylsulfonyl) ethane |

### Intermediate 405: 5-bromo-1-methyl-2-oxo-1,2-dihydropyridine-3-carboxylic acid

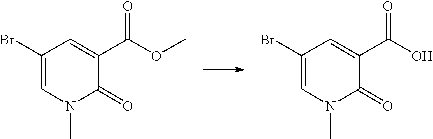

Methyl5-bromo-1-methyl-2-oxo-1,2-dihydropyridine-3-carboxylate (Intermediate 366, 2.00 g, 8.13 mmol) was dissolved in MeOH (40 mL) and treated with sodium hydroxide (16.26 mL, 16.26 mmol). The reaction was stirred at room temperature for 2 hrs. The reaction was neutralized with 1 N HCl and the solvent was removed at reduced pressure. The residue was suspended in water and filtered to afford 5-bromo-1-methyl-2-oxo-1,2-dihydropyridine-3-carboxylic acid (1.77 g).

MS (ES): (M) 232 and 234 (M+2) for C₇H₆BrNO₃.

¹H NMR (300 MHz, DMSO-d₆) δ ppm 3.62 (s, 3H) 8.35 (d, J=2.83 Hz, 1H) 8.61 (d, J=2.83 Hz, 1H) 14.38 (s, 1H)

The following compounds were prepared using the general method described for Intermediate 365 using the starting materials (SM) indicated.

| Int | Compound | Data | SM |
|---|---|---|---|
| Intermediate 406 | 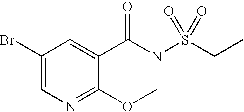 5-Bromo-N-(ethylsulfonyl)-2- methoxynicotinamide | MS(ES): 323 (M) and 325 (M + 2) for C₉H₁₁BrN₂O₄S ¹H NMR (300 MHz, DMSO- d₆) δ ppm 1.28 (t, 3 H) 3.47 (q, J = 7.35 Hz, 2 H) 3.93 (s, 3 H) 8.15 (d, J = 2.45 Hz, 1 H) 8.47 (d, J = 2.45 Hz, 1 H) 11.88 (br. s., 1 H) | Ethanesulfonamide and 5-bromo-2- methoxynicotinic acid |
| Intermediate 407 | 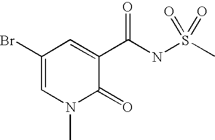 5-Bromo-1-methyl-N-(methylsulfonyl)-2- oxo-1,2-dihydropyridine-3- carboxamide | MS(ES) 309 (M) and 311 (M + 2) for C₈H₉BrN₂O₄S ¹H NMR (300 MHz, DMSO- d₆) δ ppm 3.38 (s, 3 H) 3.60 (s, 3 H) 8.39 (d, J = 2.83 Hz, 1 H) 8.63 (d, J = 2.83 Hz, 1 H) 12.65 (s, 1 H) | Methanesulfon- amide and 5-bromo-1- methyl-2-oxo- 1,2- dihydropyridine- 3-carboxylic acid Intermediate 405 |

The following compounds were prepared using the general method described above for Intermediate 134 using bis(pinacolato)diboron, 1,1′-bis(diphenylphosphino)ferrocene-palladium dichloride, potassium acetate and the starting material (SM) indicated.

| Int | Compound | Data | SM |
|---|---|---|---|
| Intermediate 408 | 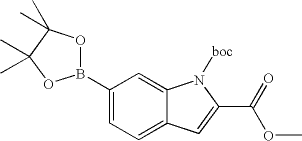 1-tert-butyl 2-methyl 6-(4,4,5,5-tetramethyl- 1,3,2-dioxaborolan-2-yl)-1H-indole-1,2- dicarboxylate | ¹H NMR (300 MHz, DMSO- D6) δ ppm 1.31 (s, 12 H) 1.55 (s, 9 H) 3.87 (s, 3 H) 7.29 (s, 1 H) 7.58 (d, J = 7.91 Hz, 1 H) 7.71 (d, J = 7.91 Hz, 1 H) 8.38 (s, 1 H) | 1-tert-butyl 2- methyl 6-bromo- 1H-indole-1,2- dicarboxylate Intermediate 386 |
| Intermediate 409 | 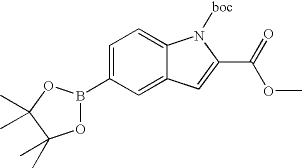 1-tert-butyl 2-ethyl 5-(4,4,5,5-tetramethyl- 1,3,2-dioxaborolan-2-yl)-1H-indole-1,2- dicarboxylate | ¹H NMR (300 MHz, DMSO- D6) δ ppm 1.23-1.42 (m, 15 H) 1.56 (s, 9 H) 4.32 (q, J = 7.16 Hz, 2 H) 7.32 (s, 1 H) 7.74 (dd, J = 8.38, 1.04 Hz, 1 H) 7.96 (d, J = 8.48 Hz, 1 H) 8.07 (s, 1 H) | 1-tert-butyl 2- ethyl 5-bromo- 1H-indole-1,2- dicarboxylate Intermediate 387 |
| Intermediate 410 | 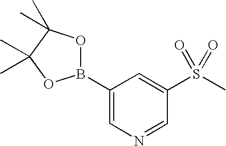 3-(Methylsulfonyl)-5-(4,4,5,5-tetramethyl- 1,3,2-dioxaborolan-2-yl)pyridine | ¹H NMR (300 MHz, DMSO- D6) δ 1.32 (s, 12 H) 3.34 (s, 3 H) 8.30-8.50 (m, 1 H) 9.01 (d, J = 1.51 Hz, 1 H) 9.15 (d, J = 2.45 Hz, 1 H) | 3-Bromo-5- (methylsulfonyl) pyridine Intermediate 390 |
| Intermediate 411 | 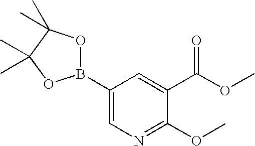 Methyl 2-methoxy-5-(4,4,5,5-tetramethyl- 1,3,2-dioxaborolan-2-yl)nicotinate | ¹H NMR (300 MHz, DMSO- D6) δ 1.29 (s, 12 H) 3.80 (s, 3 H) 3.95 (s, 3 H) 8.29 (d, J = 1.88 Hz, 1 H) 8.54 (d, J = 2.07 Hz, 1 H) | Methyl 5- bromo-2- methoxynicotinate |
| Intermediate 412 | 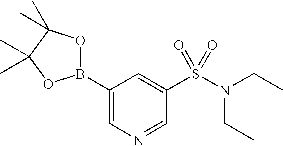 N,N-diethyl-5-(4,4,5,5-tetramethyl-1,3,2- dioxaborolan-2-yl)pyridine-3- sulfonamide | ¹H NMR (300 MHz, DMSO- d₆) δ ppm 1.06 (t, J = 7.16 Hz, 6 H) 1.33 (s, 12 H) 3.20 (q, J = 7.03 Hz, 4 H) 7.94 (s, 1 H) 8.22 (s, 1 H) 8.96 (s, 1 H) | 5-Bromo-N,N- diethylpyridine- 3-sulfonamide |
| Intermediate 413 | 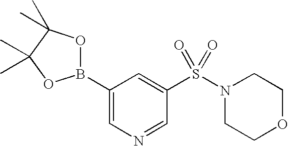 4-(5-(4,4,5,5-Tetramethyl-1,3,2- dioxaborolan-2-yl)pyridin-3- ylsulfonyl)morpholine | ¹H NMR (300 MHz, DMSO- d₆) δ ppm 1.33 (s, 12 H) 2.81- 3.08 (m, 4 H) 3.60-3.77 (m, 4 H) 7.94 (s, 1 H) 8.16 (s, 1 H) 9.03 (s, 1 H) | 4-(5- Bromopyridin-3- ylsulfonyl)- morpholine |
| Intermediate 414 | 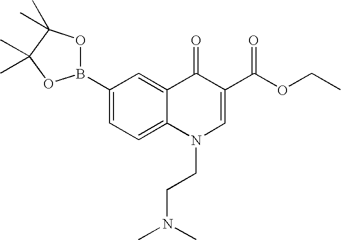 Ethyl 1-(2-(dimethylamino)ethyl)- 4-oxo-6-(4,4,5,5-tetramethyl-1,3,2- dioxaborolan-2-yl)-1,4-dihydroquinoline-3- carboxylate | ¹H NMR (300 MHz, DMSO- d₆) δ 1.21-1.35 (m, 15 H) 2.20 (s, 6 H) 2.54-2.68 (m, 2 H) 4.23 (q, J = 6.97 Hz, 2 H) 4.38-4.59 (m, 2 H) 7.79 (d, J = 8.85 Hz, 1 H) 7.90- 8.00 (m, 1 H) 8.53-8.65 (m, 2 H) | Ethyl 1-(2- (dimethylamino) ethyl)-6-iodo-4- oxo-1,4- dihydroquinoline- 3-carboxylate Intermediate 388 |
| Intermediate 415 | 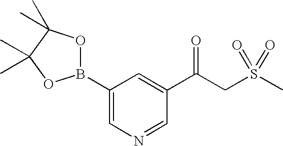 2-(Methylsulfonyl)-1-(5-(4,4,5,5-tetramehtyl- 1,3,2-dioxaborolan-2-yl)pyridin-3- yl)ethanone | ¹H NMR (300 MHz, DMSO- d₆) δ ppm 1.34 (s, 12 H) 3.14 (s, 3 H) 5.22 (br. s., 2 H) 8.49 (br. s., 1 H) 8.98 (br. s., 1 H) 9.28 (br. s., 1 H) | 1-(5- Bromopyridin-3- yl)-2- (methylsulfonyl) ethanone |
| Intermediate 416 | 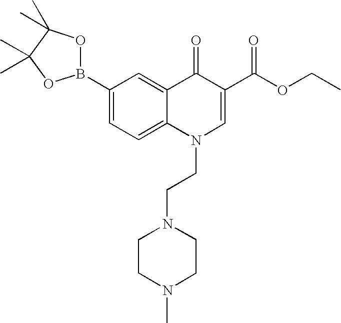 Ethyl 1-(2-(4-methylpiperazin-1- yl)ethyl)-4-oxo-6-(4,4,5,5-tetramethyl- 1,3,2-dioxaborolan-2-yl)-1,4- dihydroquinoline-3-carboxylate | ¹H NMR (300 MHz, DMSO- d₆) δ ppm 1.22-1.40 (m, 15 H) 2.02-2.47 (m, 11 H) 2.56- 2.76 (m, 2 H) 4.12-4.33 (m, 2 H) 4.32-4.61 (m, 2 H) 7.71-8.71 (m, 4 H) | Ethyl 6-iodo-1- (2-(4- methylpiperazin- 1-yl)ethyl)-4- oxo-1,4- dihydroquinoline- 3-carboxylate Intermediate 389 |
| Intermediate 417 | 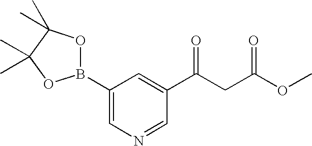 Methyl 3-oxo-3-(5-(4,4,5,5-tetramethyl- 1,3,2-dioxaborolan-2-yl)pyridin-3- yl)propanoate | ¹H NMR (300 MHz, DMSO- d₆) δ ppm 1.33 (s, 12 H) 3.65 (s, 3 H) 4.33 (s, 2 H) 8.42 (s, 1 H) 8.98 (br. s., 1 H) 9.23 (br. s., 1 H) | Methyl 5- bromonicotinoyl acetate |
| Intermediate 418 | 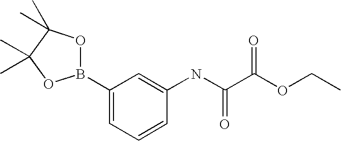 Ethyl 2-oxo-2-(3-(4,4,5,5-tetramethyl- 1,3,2-dioxaborolan-2-yl)phenylamino)acetate | ¹H NMR (300 MHz, DMSO- d₆) δ ppm 1.23-1.42 (m, 15 H) 4.31 (q, J = 7.10 Hz, 2 H) 7.25-7.48 (m, 2 H) 7.76- 7.87 (m, 1 H) 7.91 (s, 1 H) 10.73 (s, 1 H) | Ethyl 2-(3- bromophenylamino)- 2-oxoacetate Intermediate 393 |
| Intermediate 419 | 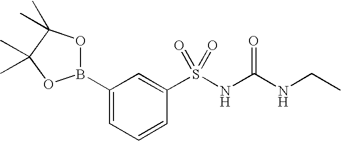 N-(ethylcarbamoyl)-3-(4,4,5,5-tetramethyl- 1,3,2-dioxaborolan-2-yl)benzenesulfonamide | MS(ES): 355 (M + 1) for C₁₅H₂₃BN₂O₅S | 3-Bromo-N- (ethylcarbamoyl)- benzenesulfonamide Intermediate 394 |
| Intermediate 420 | 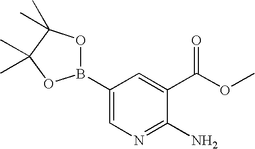 Methyl 2-amino-5-(4,4,5,5-tetramethyl- 1,3,2-dioxaborolan-2-yl)nicotinate | ¹H NMR (300 MHz, DMSO- d₆) δ ppm 1.28 (s, 12 H) 3.83 (s, 3 H) 7.50 (s, 2 H) 8.28 (d, J = 1.88 Hz, 1 H) 8.38 (d, J = 2.07 Hz, 1 H) | Methyl 2-amino- 5-bromo nicotinate |
| Intermediate 421 | 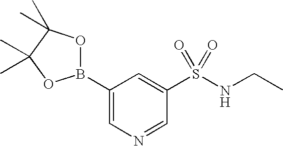 N-ethyl-5-(4,4,5,5-tetramethyl-1,3,2- dioxaborolan-2-yl)pyridine-3- sulfonamide | ¹H NMR (300 MHz, DMSO- d₆) δ ppm 0.98 (t, 3 H) 1.21- 1.45 (m, 12 H) 2.66-2.90 (m, 2 H) 7.84 (t, J = 5.46 Hz, 1 H) 8.23-8.40 (m, 1 H) 8.94 (d, J = 1.13 Hz, 1 H) 9.01 (d, J = 2.26 Hz, 1 H) | 5-Bromo-N- ethylpyridine-3- sulfonamide Intermediate 395 |
| Intermediate 422 | 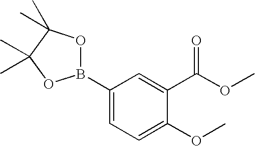 Methyl 2-methoxy-5-(4,4,5,5-tetramethyl- 1,3,2-dioxaborolan-2-yl)benzoate | ¹H NMR (300 MHz, DMSO- d₆) δ ppm 1.29 (s, 12 H) 3.78 (s, 3 H) 3.85 (s, 3 H) 7.16 (d J = 8.48 Hz, 1 H) 7.81 (dd, J = 8.38, 1.98 Hz, 1 H) 7.96 (d, J = 1.70 Hz, 1 H) | Methyl 5-iodo- 2- methoxybenzoate |
| Intermediate 423 | 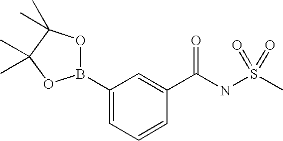 N-(methylsulfonyl)-3-(4,4,5,5-tetramethyl- 1,3,2-dioxaborolan-2-yl)benzamide | ¹H NMR (300 MHz, CHLOROFORM-d) δ ppm 1.38 (s, 12 H) 3.71 (s, 3 H) 7.53 (t, J = 7.63 Hz, 1 H) 7.98- 8.09 (m, 2 H) 8.16 (s, 1 H) | 3-Bromo-N- (methylsulfonyl) benzamide Intermediate 396 |
| Intermediate 424 | 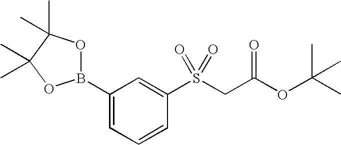 tert-butyl 2-(3-(4,4,5,5-tetramethyl-1,3,2- dioxaborolan-2-yl)phenylsulfonyl)- acetate | ¹H NMR (300 MHz, CHLOROFORM-d) δ ppm 1.36 (d, J = 5.27 Hz, 21 H) 4.05 (s, 2 H) 7.58 (t, J = 7.63 Hz, 1 H) 7.97-8.16 (m, 2 H) 8.38 (s, 1 H) | tert-Butyl 2-(3- bromophenylsul- fonyl)acetate Intermediate 392 |
| Intermediate 425 | 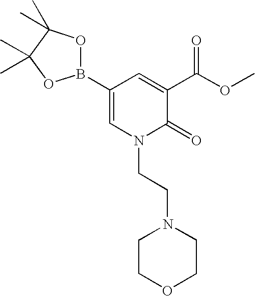 Methyl 1-(2-morpholinoethyl)-2- oxo-5-(4,4,5,5-tetramethyl-1,3,2- dioxaborolan-2-yl)-1,2-dihydropyridine-3- carboxylate | ¹H NMR (300 MHz, DMSO- d₆) δ ppm 1.28 (s, 12 H) 2.33- 2.46 (m, 4 H) 2.46-2.60 (m, 2 H) 3.42-3.55 (m, 4 H) 3.74 (s, 3 H) 4.09 (t, J = 6.03 Hz, 2 H) 8.12 (d, J = 2.26 Hz, 1 H) 8.19 (d, J = 2.07 Hz, 1 H) | Methyl 5- bromo-1-(2- morpholinoethyl)- 2-oxo-1,2- dihydropyridine- 3-carboxylate Intermediate 398 |
| Intermediate 426 | 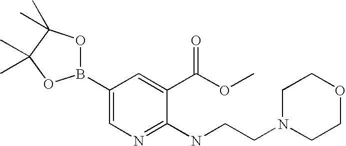 Methyl 2-(2-morpholinoethylamino)- 5-(4,4,5,5-tetramethyl-1,3,2-dioxaborolan-2- yl)nicotinate | ¹H NMR (300 MHz, DMSO- d₆) δ ppm 1.21-1.36 (m, 12 H) 2.29-2.66 (m, 6 H) 3.42- 3.72 (m, 6 H) 3.84 (s, 3 H) 8.29 (d, J = 2.07 Hz, 1 H) 8.33- 8.54 (m, 2 H) | methyl 5-bromo- 2-(2- morpholinoethyl amino)nicotinate Intermediate 399 |
| Intermediate 427 | 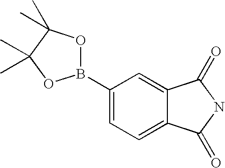 5-(4,4,5,5-Tetramethyl-1,3,2-dioxaborolan-2- yl)isoindoline-1,3-dione | ¹H NMR (300 MHz, DMSO- d₆) δ ppm 1.33 (s, 12 H) 7.83 (d, J = 7.35 Hz, 1 H) 7.96 (s, 1 H) 8.08 (dd, J = 7.35, 0.75 Hz, 1 H) 11.46 (br. s., 1 H) | 4- bromophthalimide |
| Intermediate 428 | 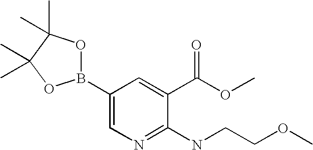 Methyl 2-(2- methoxyethylamino)-5- (4,4,5,5-tetramethyl- 1,3,2-dioxaborolan-2- yl)nicotinate | ¹H NMR (300 MHz, DMSO- d₆) δ ppm 1.28 (s, 12 H) 3.29 (s, 3 H) 3.51 (t, J = 5.46 Hz, 2 H) 3.66 (q, J = 5.46 Hz, 2 H) 3.83 (s, 3 H) 8.23-8.37 (m, 2 H) 8.45 (d, J = 1.88 Hz, 1 H) | Methyl 5- bromo-2-(2- methoxyethyl- amino)nicotinate Intermediate 400 |
| Intermediate 429 | 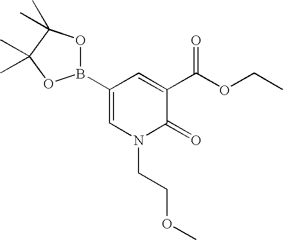 Ethyl 1-(2-methoxyethyl)-2-oxo- 5-(4,4,5,5-tetramethyl-1,3,2-dioxaborolan-2- yl)-1,2-dihydropyridine-3- carboxylate | ¹H NMR (300 MHz, DMSO- d₆) δ 1.21-1.38 (m, 15 H) 3.23 (s, 3 H) 3.50-3.66 (m, 2 H) 4.09-4.28 (m, 4 H) 8.08 (d, J = 2.07 Hz, 1 H) 8.12 (d, J = 2.26 Hz, 1 H) | Ethyl 5-bromo- 1-(2- methoxyethyl)- 2-oxo-1,2- dihydropyridine- 3-carboxylate Intermediate 401 |
| Intermediate 430 | 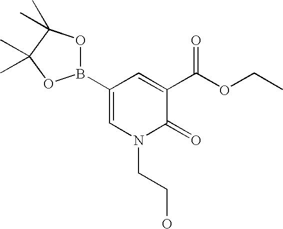 Ethyl 1-(2-hydroxyethyl)-2-oxo-5- (4,4,5,5-tetramethyl-1,3,2-dioxaborolan-2- yl)-1,2-dihydropyridine-3- carboxylate | ¹H NMR (300 MHz, DMSO- d₆) δ ppm 1.21-1.35 (m, 15 H) 3.62 (q, J = 5.15 Hz, 2 H) 4.03 (t, J = 4.99 Hz, 2 H) 4.21 (q, J = 7.10 Hz, 2 H) 4.86 (t, J = 5.46 Hz, 1 H) 8.03-8.18 (m, 2 H) | Ethyl 5-bromo- 1-(2- hydroxyethyl)-2- oxo-1,2- dihydropyridine- 3-carboxylate Intermediate 402 |
| Intermediate 431 | 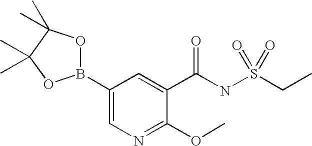 N-(ethylsulfonyl)-2-methoxy-5-(4,4,5,5- tetramethyl-1,3,2-dioxaborolan-2- yl)nicotinamide | ¹H NMR (300 MHz, DMSO- d₆) δ ppm 1.20-1.40 (m, 15 H) 3.45 (q, J = 7.16 Hz, 2H) 3.96 (s, 3 H) 8.05 (d, J = 1.88 Hz, 1 H) 8.51 (d, J = 1.88 Hz, 1 H) 11.75 (s, 1 H) | 5-Bromo-N- (ethylsulfonyl)- 2- methoxynicotin- amide Intermediate 406 |
| Intermediate 432 | 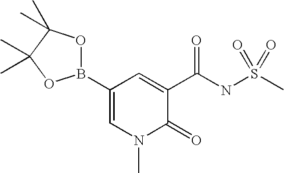 1-Methyl-N-(methylsulfonyl)-2- oxo-5-(4,4,5,5-tetramethyl-1,3,2- dioxaborolan-2-yl)-1,2-dihydropyridine-3- carboxamide | ¹H NMR (300 MHz, DMSO- d₆) δ ppm 1.30 (s, 12 H) 3.36 (br. s., 3 H) 3.63 (s, 3 H) 8.31-8.61 (m, 2 H) 12.55 (br. s., 1 H) | 5-Bromo-1- methyl-N- (methylsulfonyl)- 2-oxo-1,2- dihydropyridine- 3-carboxamide Intermediate 407 |
| Intermediate 433 | 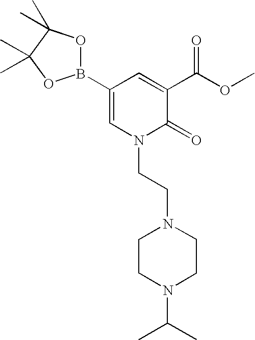 Methyl 1-(2-(4-isopropylpiperazin-1- yl)ethyl)-2-oxo-5-(4,4,5,5-tetramethyl- 1,3,2-dioxaborolan-2-yl)-1,2- dihydropyridine-3-carboxylate | ¹H NMR (300 MHz, DMSO- d₆) δ ppm 0.93 (d, J = 6.59 Hz, 6 H) 1.21-1.29 (m, 12 H) 1.79 (br. s., 2 H) 2.29- 2.44 (m, 8 H) 2.52-2.66 (m, 1 H) 3.73 (s, 3 H) 4.07 (t, J = 5.84 Hz, 2 H) 8.05-8.19 (m, 2 H) | Methyl 5- bromo-1-(2-(4- isopropylpiper- azin-1-yl)ethyl)-2- oxo-1,2- dihydropyridine- 3-carboxylate Intermediate 403 |
| Intermediate 434 | 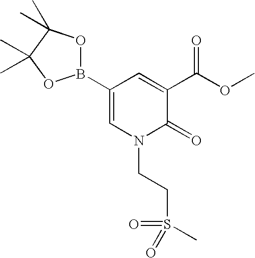 Methyl 1-(2-(methylsulfonyl)ethyl)- 2-oxo-5-(4,4,5,5-tetramethyl-1,3,2- dioxaborolan-2-yl)-1,2-dihydropyridine-3- carboxylate | ¹H NMR (300 MHz, DMSO- d₆) δ ppm 1.29 (s, 12 H) 3.07 (s, 3 H) 3.56 (t, J = 6.88 Hz, 2 H) 3.75 (s, 3 H) 4.38 (t, J = 6.97 Hz, 2 H) 8.14 (d, J = 2.07 Hz, 1 H) 8.32 (d, J = 2.07 Hz, 1 H) | Methyl 5- bromo-1-(2- (methylsulfonyl) ethyl)-2-oxo- 1,2- dihydropyridine- 3-carboxamide Intermediate 404 |

### Intermediate 435: 5-bromo-N-butan-2-yl-2-chloropyrimidin-4-amine

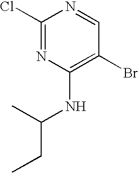

Prepared using the general method described above for Intermediate 1 using 5-bromo-2,4-dichloro-pyrimidine and butan-2-amine. MS(ES): 265.8 (M+2) for C₈H₁₁BrClN₃.

1H NMR (300 MHz, CHLOROFORM-D) δ ppm 0.93-1.03 (m, 3H), 1.26 (d, J=6.59 Hz, 3H), 1.55-1.70 (m, 2H), 4.06-4.36 (m, 1H), 5.30 (s, 1H), 8.12 (s, 1H).

### Intermediate 436: 5-bromo-N′-butan-2-yl-N-(3-chloro-4-fluorophenyl)pyrimidine-2,4-diamine

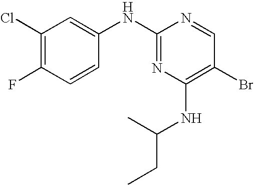

Prepared using the general method described above for Intermediate 26 using Intermediate 435 and 3-chloro-4-fluoroaniline.

MS(ES): 375 (M+2) for C₁₄H₁₅BrClFN₄.

1H NMR (300 MHz, CHLOROFORM-D) δ ppm 0.98-1.03 (m, 3H), 1.29 (d, J=6.59 Hz, 3H), 1.55-1.70 (m, 2H), 4.06-4.30 (m, 1H), 5.17 (m, 1H), 7.07 (m, 1H), 7.23 (m, 2H), 7.97-8.00 (m, 2H).

The following intermediates were prepared using the general method described above for Intermediate 112 using Intermediate 69 and the starting material (SM) indicated.

| Int | Compoun d | Data | SM |
|---|---|---|---|
| Intermediate 437 | 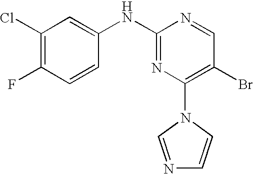 5-bromo-N-(3-chloro-4- fluorophenyl)-4-imidazol-1- ylpyrimidin-2-amine | MS(ES): 370 (M + 2) for C₁₃H₈BrClFN₅. 1H NMR (300 MHz, DMSO-D6) δ ppm 7.18 (s, 1 H) 7.40 (t, J = 9.14 Hz, 1 H), 7.58-7.67 (m, 1 H), 7.82 (s, 1 H), 7.98 (dd, J = 6.78, 2.64 Hz, 1 H) 8.39 (s, 1 H), 8.86 (s, 1 H) 10.33 (s, 1 H). | imidazole |
| Intermediate 438 | 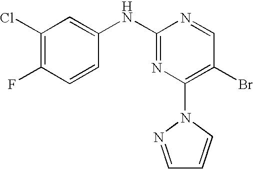 5-bromo-N-(3-chloro-4- fluorophenyl)-4-pyrazol-1- ylpyrimidin-2-amine | MS(ES): 369.9 (M + 2) for C₁₃H₈BrClFN₅. 1H NMR (300 MHz, DMSO-D6) δ ppm 6.67 (dd, J = 2.64, 1.70 Hz, 1 H), 7.40 (t, J = 9.14 Hz, 1 H) 7.57-7.73 (m, 1 H) 7.94 (d, J = 0.94 Hz, 1 H), 8.00 (dd, J = 6.78, 2.64 Hz, 1 H), 8.47 (d, J = 2.07 Hz, 1 H), 8.83 (s, 1 H), 10.28 (s, 1 H). | pyrazole |

The following intermediates were prepared using the general method described above for Intermediate 65 using 5-bromo-2-chloro-4-(methylthio)pyrimidine and the starting material (SM) indicated.

| Int | Compound | Data | SM |
|---|---|---|---|
| Intermediate 439 | 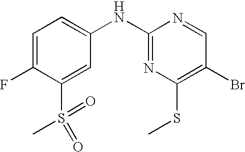 5-bromo-N-(4-fluoro-3- methylsulfonyl-phenyl)-4- methylsulfanyl-pyrimidin-2-amine | MS(ES): 393.8 (M + 2) for C₁₂H₁₁BrFN₃O₂S₂. 1H NMR (300 MHz, DMSO-D6) δ ppm 2.59 (s, 3 H) 3.32 (s, 3 H), 7.35- 7.61 (m, 1 H), 7.83-8.02 (m, 1 H), 8.36 (s, 1 H), 8.46 (dd, J = 6.22, 2.83 Hz, 1 H), 10.16 (s, 1 H). | 4-fluoro-3- (methyl- sulfonyl)aniline |
| Intermediate 440 | 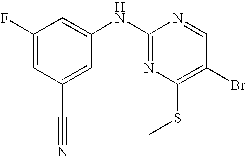 3-(5-bromo-4-(methylthio)- pyrimidin-2-ylamino)-5- fluorobenzonitrile | MS(ES): 340.8 (M + 2) for C₁₂H₈BrFN₄S. 1H NMR (300 MHz, DMSO-D6) δ ppm 2.60 (s, 3 H), 7.32-7.49 (m,1 H), 7.93-8.00 (m, 1 H), 8.02 (t, J = 1.60 Hz, 1 H), 8.42 (s, 1 H), 10.31 (s, 1 H). | 3-amino-5- fluorobenzonitrile |
| Intermediate 441 | 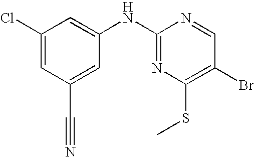 3-(5-bromo-4-(methylthio)- pyrimidin-2-ylamino)-5- chlorobenzonitrile | MS(ES): 357 (M + 2) for C₁₂H₈BrClN₄S. 1H NMR (300 MHz, DMSO-D6) δ ppm 2.60 (s, 3 H) 7.48-7.68 (m, 1 H), 8.08-8.14 (m, 1 H), 8.19 (t, J = 1.98 Hz, 1 H), 8.43 (s, 1 H), 10.29 (s, 1 H). | 3-amino-5- chlorobenzonitrile |

The following intermediates were prepared using the general method described above for Intermediate 69 using m-CPBA and the starting material (SM) indicated.

| Int | Compound | Data | SM |
|---|---|---|---|
| Intermediate 442 | 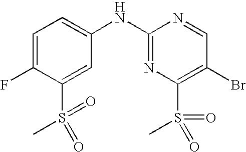 5-bromo-N-(4-fluoro-3- (methylsulfonyl)-phenyl)-4- (methylsulfonyl)-pyrimidin-2-amine | MS(ES): 426 (M + 2) for C₁₂H₁₁BrFN₃O₄S₂. 1H NMR (300 MHz, DMSO-D6) δ ppm 3.34 (s, 3 H), 3.45-3.52 (m, 3 H), 7.53 (t, J = 9.42 Hz, 1 H), 7.85- 8.02 (m, 1 H), 8.30 (dd, J = 6.12, 2.73 Hz, 1 H), 8.96 (s, 1 H), 10.70 (s, 1 H). | Intermediate 439 |
| Intermediate 443 | 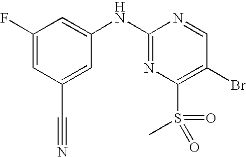 3-(5-bromo-4-(methylsulfonyl)- pyrimidin-2-ylamino)-5- fluorobenzonitrile | MS(ES): 372.7 (M + 2) for C₁₂H₈BrFN₄O₂S. 1H NMR (300 MHz, DMSO-D6) δ ppm 3.49 (s, 3 H), 7.41-7.58 (m, 1 H), 7.89-8.07 (m, 2 H), 9.03 (s, 1 H), 10.88 (s, 1 H). | Intermediate 440 |
| Intermediate 444 | 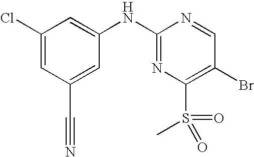 3-(5-bromo-4-(methylsulfonyl)- pyrimidin-2-ylamino)-5- chlorobenzonitrile | MS(ES): 389 (M + 2) for C₁₂H₈BrClN₄O₂S. | Intermediate 441 |

The following intermediates were prepared using the general method described above for Intermediate 112 using the starting materials (SM) indicated.

| Int | Compound | Data | SM |
|---|---|---|---|
| Intermediate 445 | 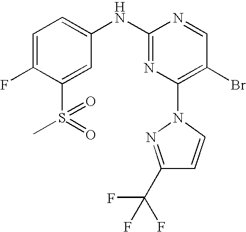 5-bromo-N-(4-fluoro-3- (methylsufonyl)-phenyl)-4-(3- (trifluoromethyl)-1H-pyrazol-1- yl)pyrimidin-2-amine | MS(ES): 481.7 (M + 2) for C₁₅H₁₀BrF₄N₅O₂S. 1H NMR (300 MHz, DMSO-D6) δ ppm 3.35 (s, 3 H), 7.15 (d, J = 2.64 Hz, 1 H), 7.52 (t, J = 9.42 Hz, 1 H), 7.84- 8.12 (m, 1 H), 8.43 (dd, J = 6.12, 2.73 Hz, 1 H), 8.68 (d, J = 1.51 Hz, 1 H), 8.93 (s, 1 H), 10.61 (s, 1H). | Intermediate 443 and 3- (trifluoro- methyl)-1H- pyrazole |
| Intermediate 446 | 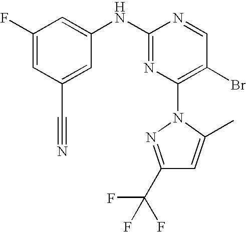 3-(5-bromo-4-(5-methyl-3- (trifluoromthyl)-1H-pyrazol-1- yl)pyrimidin-2-ylamino)-5- fluorobenzonitrile | MS(ES): 443 (M + 2) for C₁₆H₉BrF₄N₆. 1H NMR (300 MHz, DMSO-D6) δ ppm 2.42 (s, 3 H), 6.88 (s, 1 H), 7.37- 7.60 (m, 1 H), 7.80-8.15 (m, 2 H), 9.07 (s, 1 H), 10.79 (s, 1 H). | Intermediate 443 and 5- methyl-3- (trifluoro- methyl)-1H- pyrazole |
| Intermediate 447 | 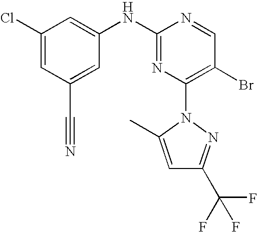 3-(5-bromo-4-(5-methyl-3- (trifluoromethyl)-1H-pyrazol-1- yl)pyrimidin-2-ylamino)-5- chlorobenzonitrile | MS(ES): 459 (M + 2) for C₁₆H₉BrClF₃N₆. 1H NMR (300 MHz, DMSO-D6) δ ppm 2.42 (s, 3 H), 6.88 (s, 1 H), 7.66 (s, 1 H), 8.00-8.23 (m, 2 H), 9.07 (s, 1 H), 10.75 (s, 1 H). | Intermediate 444 and 5- methyl-3- (trifluoro- methyl)-1H- pyrazole |

### Intermediate 448: 5-bromo-N-(3,5-dimethoxyphenyl)-4-hydrazinylpyrimidin-2-amine

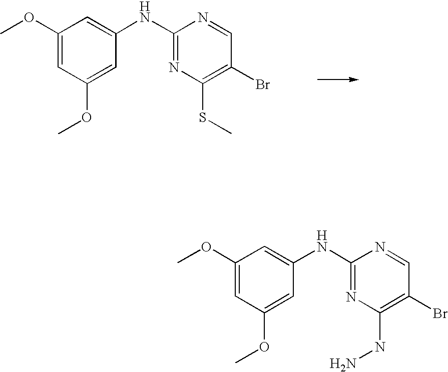

5-bromo-N-(3,5-dimethoxyphenyl)-4-(methylthio)pyrimidin-2-amine Intermediate 363 (1 g, 2.81 mmol), hydrazine anhydrous (12 ml, 382.34 mmol) and dioxane (4 mL) were combined to give a white suspension. The reaction mixture was heat at 100° C. for 2 hours. The reaction mixture was cooled over an ice bath and 60 ml of water was slowly added. The precipitated solid was filtered and washed with water to give the crude title compound (462 mg), used without further purification in the next step.

MS (Electrospray): 341.18 (MH⁺) for C₁₂H₁₄BrN₅O₂

### Intermediate 449: 5-bromo-4-(3-(difluoromethyl)-5-methyl-1H-pyrazol-1-yl)-N-(3,5-dimethoxyphenyl)pyrimidin-2-amine

### Intermediated 450: 5-bromo-4-(5-(difluoromethyl)-3-methyl-1H-pyrazol-1-yl)-N-(3,5dimethoxyphenyl)pyrimidin-2-amine

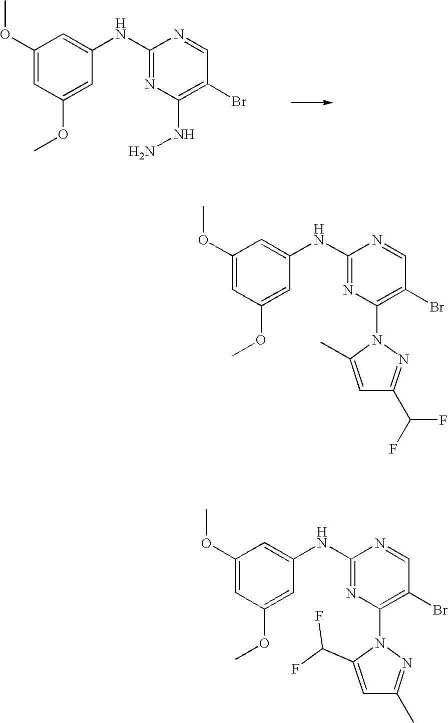

5-bromo-N-(3-chloro-4-fluorophenyl)-4-hydrazinylpyrimidin-2-amine (487 mg, 1.43 mmol), 1,1-difluoropentane-2,4-dione (214 mg, 1.57 mmol), acetic acid (0.082 mL, 1.43 mmol) and butan-1-ol (4 mL) were combined to give a yellow suspension. The mixture was heated at 150° C. for one hour. The resulting mixture of two product isomers was separated using flash chromatography, silica gel, 5-45% ethyl acetate in hexanes.

Intermediate 449 was isolated as a solid (210 mg).

Intermediate 450 was isolated as a solid (102 mg).

MS:ES+ 440 for C₁₇H₁₆BrF₂N₅O₂

These materials gave the same mass spectral results and were taken onto the next step without further characterization. The assigned regioisomeric identities of these intermediates were based on NMR analysis of the products obtained in the next reactions.

### Intermediate 451: tert-butyl N-[(2-methylpropan-2-yl)oxycarbonyl]-N-(4-morpholin-4-ylpyridin-2-yl)carbamate

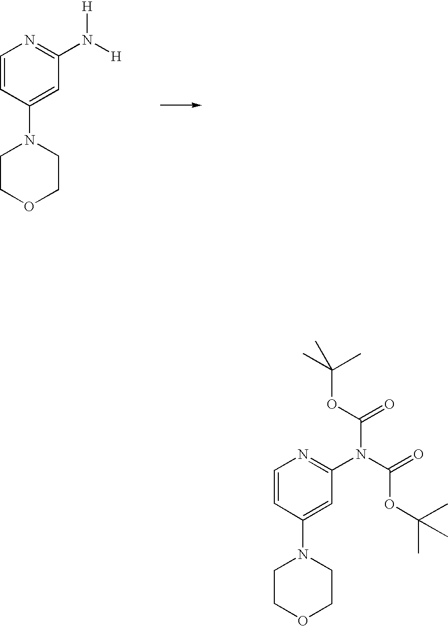

4-morpholinopyridin-2-amine (prepared according to literature procedure: Bioorganic & Medicinal Chemistry Letters, 16(4), 839-844, 2006) (1.082 g, 6.04 mmol), di-t-butyl-dicarbonate (1.542 mL, 6.64 mmol), and TEA (1.010 mL, 7.24 mmol) were combined in Dioxane (20 mL) to give a colorless solution. DMAP (0.738 g, 6.04 mmol) was added and the mixture allowed to stir at RT for 2 hours, then heated to 70° C. for 1 hour. An additional amount of di-t-butyl-dicarbonate (2 g, 9.17 mmol) was added and the mixture was heated at 70° C. overnight. The reaction mixture was concentrated and diluted with methylene chloride and water. The layers were separated and the organic layer was washed with water, dried over Na₂SO₄ then concentrated. The resulting residue was triturated with diethyl ether, filtered and further washed with diethyl ether to give the pure title compound. (1.654 g).

MS (Electrospray): 380.45 (MH⁺) for C₁₉H₂₉N₃O₅

### Intermediate 452: tert-butyl N-(5-bromo-4-morpholin-4-ylpyridin-2-yl)-N-[2-methylpropan-2-yl)oxycarbonyl]carbamate

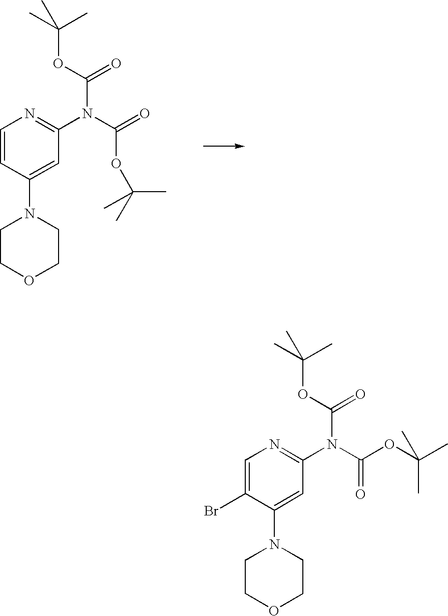

tert-butyl N-R2-methylpropan-2-yl)oxycarbonyl]-N-(4-morpholin-4-ylpyridin-2-yl)carbamate Intermediate 451 (51 mg, 0.29 mmol) was dissolved in DMF (20 mL). N-bromosuccinimide (51.6 mg, 0.29 mmol) was added and the mixture was heated to 85° C. for 1 hour. The mixture was concentrated and purified by flash chromatography (silica gel column, 40 g, eluted with 0-40% ethyl acetate in hexanes) to give the title compound. (95 mg). MS (Electrospray): 459.35 (MH⁺) for C₁₉H₂₈BrN₃O₅

### Intermediate 453: 5-bromo-4-morpholinopyridin-2-amine

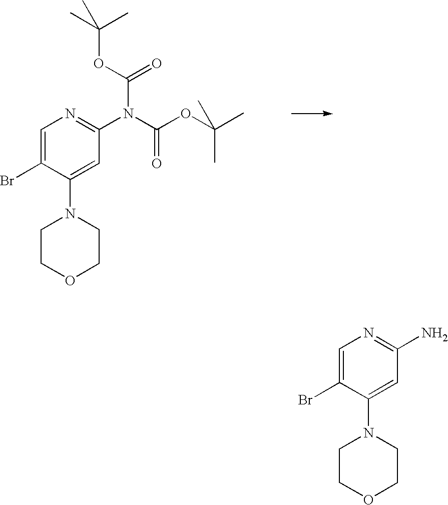

tert-butyl N-(5-bromo-4-morpholin-4-ylpyridin-2-yl)-N-[2-methylpropan-2-yl)oxycarbonyl]carbamate Intermediate 452 (1.632 g, 3.56 mmol) was dissolved in anhydrous MeOH (10 ml) and HCl 4M in Dioxane (2.67 ml, 10.68 mmol) was added. The mixture was allowed to stir at rt overnight, then heat at 50° C. for 7 hours. The reaction mixture was concentrated to give the crude title compound (926 mg) which was used in the next step without further purification. MS (Electrospray): 259.12 (MH⁺) for C₉H₁₂BrN₃O

### Intermediate 454: 5-bromo-N-(3-chloro-4-fluorophenyl)-4-morpholinopyridin-2-amine

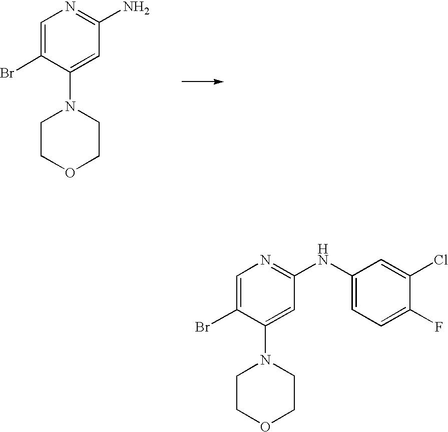

5-bromo-4-morpholinopyridin-2-amine Intermediate 453 (320 mg, 0.97 mmol), 3-chloro-4-fluorophenylboronic acid (337 mg, 1.93 mmol), and 5-bromo-4-morpholinopyridin-2-amine (320 mg, 0.97 mmol) were combined with methylene chloride (5 ml) to give a yellow solution. Copper(II)acetate (176 mg, 0.97 mmol) was added followed by anhydrous powdered 3 angstrom molecular sieves (200 mg). The mixture was allowed to stir at rt overnight. Filtration, evaporation and purification by flash chromatography (0-100% ethyl acetate in hexanes) afforded the title compound (82 mg). MS (Electrospray): 387.65 (MH⁺) for C₁₅H₁₄BrClFN₃O

### Intermediate 455: 5-bromo-2-(2-methoxyethoxy)nicotinic acid

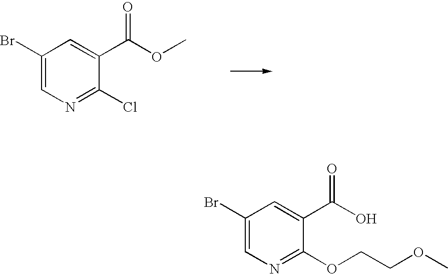

methyl 5-bromo-2-chloronicotinate (500 mg, 2.00 mmol) and 2-methoxyethanol (456 mg, 5.99 mmol) were combined in tert-butanol (20 mL) to give a yellow solution. Sodium 2-methylpropan-2-olate (576 mg, 5.99 mmol) was added. The mixture was heated at 90° C. for 1 hour then concentrated. 1 N HCl was added to the residue followed by extraction with ethyl acetate. The organic layer was washed with water then brine, dried with MgSO₄ and concentrated to give the title compound (454 mg). MS (Electrospray): 277.08 (MH⁺) for C₉H₁₀BrNO₄

The compounds in the table below were prepared using this procedure and the specified starting materials.

| | | Mass spectrum and ¹H | |
|---|---|---|---|
| Compound | Structure | NMR | SM |
| Intermediate 456 | 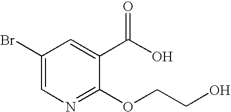 5-bromo-2-(2-hydroxyethoxy)nico- tinic acid | MS(ES): 263.06 (M + 1) for C₈H₈BrNO₄ | 5-bromo-2- chloronico- tinic acid and ethylene glycol |
| Intermediate 457 | 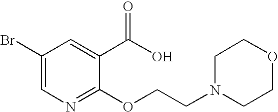 5-bromo-2-(2-morpholinoethoxy)nico- tinic acid | MS(ES): 331.16 (M + 1) for C₁₂H₁₅BrN₂O₄ | 5-bromo-2- chloronico- tinic acid and 2- morpholino ethanol |
| Intermediate 458 | 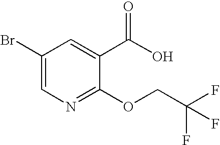 5-bromo-2-(2,2,2-trifluoroethoxy)nico- tinic acid | MS(ES): 301.03 (M + 1) for C₈H₅BrF₃NO₃ | 5-bromo-2- chloronico- tinic acid and 2,2,2-trifluoro- ethanol |
| Intermediate 459 | 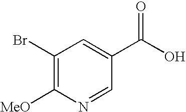 5-bromo-6-methoxynicotinic acid | MS(ES): 232.03 (M + 1) for C₇H₆BrNO₃ | 5-bromo-2- chloronico- tinic acid and methanol |

### Intermediate 460: methyl5-bromo-2-(2-methoxyethoxy)nicotinate

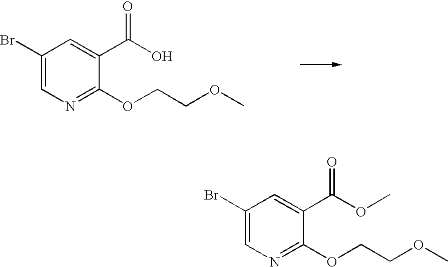

5-bromo-2-(2-methoxyethoxy)nicotinic acid Intermediate 455 (454 mg, 1.64 mmol) was dissolved in anhydrous methanol (20 mL) and H₂SO₄ (0.088 mL, 1.64 mmol) was added to give a brown solution. The mixture was stirred overnight at room temperature. An additional amount of H₂SO₄ (0.088 mL, 1.64 mmol) was added and the mixture was heated at 60° C. for 5 hours. The reaction mixture was concentrated down to dryness, ethyl acetate followed by water was added and the layers were separated. The organic layer was washed with water, then brine and dried over MgSO₄. Evaporation gave the title compound (440 mg). MS (Electrospray): 291.11 (MH⁺) for C₁₀H₁₂BrNO₄

The compounds in the table below were prepared using this procedure and the specified starting materials.

| | | Mass spectrum and ¹H | |
|---|---|---|---|
| Compound | Structure | NMR | SM |
| Intermediate 461 | 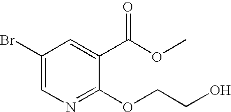 methyl 5-bromo-2-(2-hydroxyethoxy)nicotinate | MS(ES): 277.08 (M + 1) for C₉H₁₀BrNO₄ | 5-bromo-2-(2- hydroxyethoxy)nicotinic acid Intermediate 456 |
| Intermediate 462 | 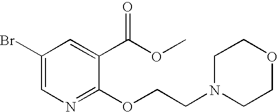 methyl 5-bromo-2-(2- morpholinoethoxy)nicotinate | MS(ES): 346.19 (M + 1) for C₁₃H₁₇BrN₂O₄ | 5-bromo-2-(2- morpholinoethoxy)nicotinic acid Intermediate 457 |
| Intermediate 463 | 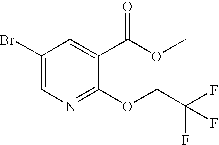 methyl 5-bromo-2-(2,2,2- trifluoroethoxy)nicotinate | MS(ES): 315.06 (M + 1) for C₉H₇BrF₃NO₃ | 5-bromo-2-(2,2,2- trifluoroethoxy)nicotinic acid Intermediate 458 |
| Intermediate 464 | 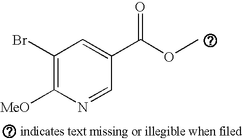 methyl 5-bromo-6-methoxynicotinate | MS(ES): 246.06 (M + 1) for C₈H₈BrNO₃ | 5-bromo-6- methoxynicotinic acid Intermediate 459 |

### Intermediate 465: methyl2-(2-methoxyethoxy)-5-(4,4,5,5-tetramethyl-1,3,2-dioxaborolan-2-yl)nicotinate and 5-(methoxycarbonyl)-6-(2-methoxyethoxy)pyridin-3-ylboronic acid

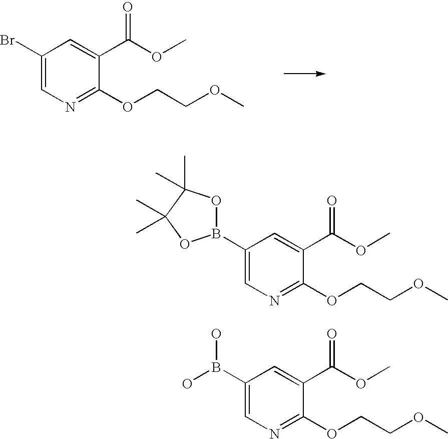

methyl5-bromo-2-(2-methoxyethoxy)nicotinate Intermediate 460 (440 mg, 1.52 mmol), bis(pinacolato)diboron (539 mg, 2.12 mmol), and potassium acetate (447 mg, 4.55 mmol) were combined in 1,4-dioxane (20 mL). PdCl2(dppf)-CH₂Cl₂ Adduct (1239 mg, 1.52 mmol) was added and the reaction was degassed with argon then heated to 90° C. for 3 hours. Purification by flash chromatography (10-100% ethyl acetate in hexanes) afforded the title compound as a mixture of boronic acid and pinacol ester (365 mg, ester: acid 1:1 mixture).

MS (Electrospray): 338.18 (MH⁺) for C₁₆H₂₄BNO₆

MS (Electrospray): 256.03 (MH⁺) for C₁₀H₁₄BNO₆

The compounds in the below table were prepared using this procedure and the specified starting materials.

| | | Mass spectrum and | |
|---|---|---|---|
| Compound | Structure | ¹H NMR | SM |
| Intermediate 466 | 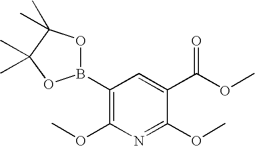 methyl 2,6-dimethoxy-5-(4,4,5,5-tetramethyl-1,3,2- dioxaborolan-2-yl)nicotinate | MS(ES): 323.15 (M) for C₁₅H₂₂BNO₆ | methyl 5- bromo-2,6- dimethoxynico- tinate |
| Intermediate 467 | 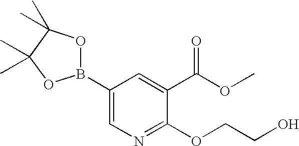 methyl 2-(2-hydroxyethoxy)-5-(4,4,5,5-tetramethyl-1,3,2- dioxaborolan-2-yl)nicotinate | MS(ES): 323.15 (M) for C₁₅H₂₂BNO₆ | methyl 5- bromo-2-(2- hydroxyethoxy)- nicotinate Intermediate 461 |
| Intermediate 468 | 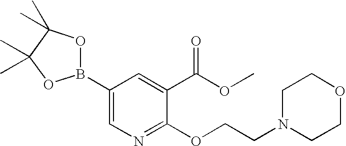 methyl 2-(2-morpholinoethoxy)-5- (4,4,5,5-tetramethyl-1,3,2-dioxaborolan-2-yl)nicotinate | MS(ES): 392.25 (M) for C₁₉H₂₉BN₂O₆ | methyl 5- bromo-2-(2- morpholino- ethoxy)nicotinate Intermediate 462 |
| Intermediate 469 | 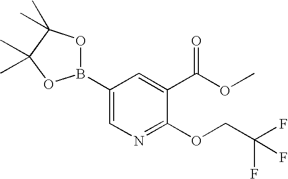 methyl 5-(4,4,5,5-tetramethyl-1,3,2- dioxaborolan-2-yl)-2-(2,2,2-trifluoroethoxy)nicotinate | MS(ES): 361.12 (M) for C₁₅H₁₉BF₃NO₅ | methyl 5- bromo-2- (2,2,2- trifluoroethoxy)- nicotinate Intermediate 463 |
| Intermediate 470 | 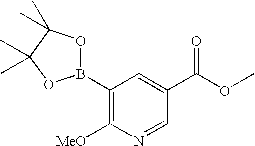 methyl 6-methoxy-5-(4,4,5,5-tetramethyl-1,3,2- dioxaborolan-2-yl)nicotinate | MS(ES): 293.12 (M) for C₁₄H₂₀BNO₅ | methyl 5- bromo-6- methoxynico- tinate Intermediate 464 |

### Intermediate 471: 5-bromo-4-(3-cyclopropyl-1H-pyrazol-1-yl)-N-(3,5-dimethoxyphenyl)pyrimidin-2-amine

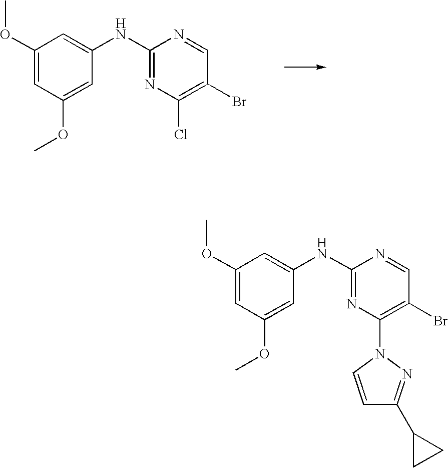

NaH (0.07 g, 1.75 mmol) was suspended in anhydrous NMP (5 mL) and cooled to 0° C., 3-cyclopropyl-1H-pyrazole (0.235 g, 2.18 mmol) was then added slowly and the mixture was stirred for 15 minutes. 5-Bromo-4-chloro-N-(3,5-dimethoxyphenyl)pyrimidin-2-amine Intermediate 213 (0.5 g, 1.45 mmol) in a solution of 10 ml of NMP under argon was added and the reaction mixture was allowed to warm to room temperature overnight. Water (60 ml) was added to give a precipitate, which was filtered, washed with water and dried under vacuum to give the title compound as an off- white solid (503 mg). MS (Electrospray): 417.27 (MH⁺) for C₁₈H₁₈BrN₅O₂

The compound below was prepared according the above procedure for Intermediate 471 using the specified starting materials.

| Compound | Structure | Mass spectrum | SM |
|---|---|---|---|
| Intermediate 472 | 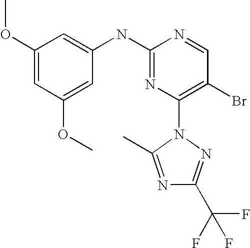 5-bromo-N-(3,5-dimethoxyphenyl)-4-(5- methyl-3-(trifluoromethyl)-1H-1,2,4-triazol-1- yl)pyrimidin-2-amine | MS: ES+ 460.22 for (M + 1) C₁₆H₁₄BrF₃N₆O₂ | 5-bromo-4- chloro-N-(3,5- dimethoxyphenyl) pyrimidin-2- amine Intermediate 213 and 5-methyl-3- (trifluoromethyl)- 1H-1,2,4-triazole |

### Intermediate 473: methyl5-bromo-6-fluoronicotinate

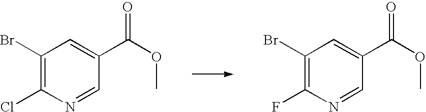

Methyl 5-bromo-6-chloronicotinate (2.885 g, 11.52 mmol), potassium fluoride (2.68 g, 46.07 mmol), and tetraphenylphosphonium bromide (2.90 g, 6.91 mmol) combined in acetonitrile (75 mL) to give a yellow suspension. The reaction mixture was warmed at reflux or overnight. Additional potassium fluoride (1 g, 17 mmol) was added and the mixture was refluxed for 5 days more. Evaporation and purification by flash chromatography (0-25% ethyl acetate in hexanes) afforded the title compound (1.53 g).

MS (Electrospray): 235.02 (MH⁺) for C₇H₅BrFNO₂

### Intermediate 474: Methyl5-bromo-6-(2-(dimethylamino)ethoxy)nicotinate

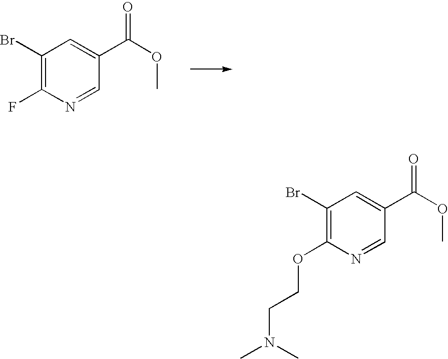

2-(dimethylamino)ethanol (503 mg, 5.64 mmol) was dissolved in THF (6 mL) and cooled to 0° C. 1M Lithium bis(trimethylsilyl)amide in THF (6.41 mL, 6.41 mmol) was added slowly and the mixture was allowed stir for 15 minutes. A solution of methyl 5-bromo-6-fluoronicotinate Intermediate 473 (600 mg, 2.56 mmol) in THF (4 ml) was then added to the reaction mixture. The reaction was allowed to reach room temperature over 4 hours, 1M NH₄Cl and dichloromethane were added and the layers were separated. The water layer was extracted with 5% methanol in methylene chloride. The pooled organic layers were dried over MgSO₄ and purified by flash chromatography (3-10% methanol in methylene chloride) to afford the title compound (312 mg).

MS (Electrospray): 304.15 (MH⁺) for C₁₁H₁₅BrN₂O₃

### Intermediate 475: methyl5-bromo-1-(2-methoxyethyl)-2-oxo-1,2-dihydropyridine-3-carboxylate

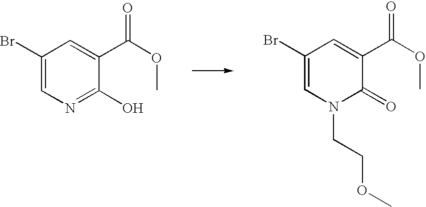

Sodium hydride (0.207 g, 5.17 mmol) was suspended in DMF (12 mL) in an oven dried flask under nitrogen and treated with methyl5-bromo-2-hydroxynicotinate (1.2 g, 5.17 mmol). The mixture was stirred at room temperature for 30 minutes then 2-Bromoethyl methylether (1.458 mL, 15.52 mmol) was added dropwise. The mixture was then heated at 60° C. overnight. The mixture was diluted with water and extracted with EtOAc. The extracts were washed with brine, dried over sodium sulfate, and the solvent removed at reduced pressure. The residue was purified by flash chromatography, silica gel, 20-100% EtOAc in DCM to afford the desired product.

| Compound | Structure | ¹H NMR | SM |
|---|---|---|---|
| Intermediate 475 | 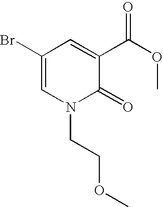 methyl 5-bromo-1-(2- methoxyethyl)-2-oxo- 1,2-dihydropyridine-3- carboxylate | 1H NMR (300 MHz, DMSO-d6) δ ppm 3.24 (s, 3 H) 3.57 (t, J = 5.27 Hz, 2 H) 3.75 (s, 3 H) 4.10 (t, J = 5.27 Hz, 2 H) 8.05 (d, J = 2.83 Hz, 1 H) 8.22 (d, J = 2.83 Hz, 1 H) | methyl 5- bromo-2- hydroxynico- tinate |

The following compounds were prepared using the general method described above for Intermediate 134 using bis(pinacolato)diboron, 1,1′-bis(diphenylphosphino)ferrocene-palladium dichloride, potassium acetate and the starting material (SM) indicated.

| | | Mass spectrum and ¹H | |
|---|---|---|---|
| Compound | Structure | NMR | SM |
| Intermediate 476 | 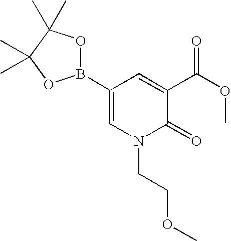 methyl 1-(2-methoxyethyl)-2-oxo- 5-(4,4,5,5-tetramethyl-1,3,2- dioxaborolan-2-yl)-1,2-dihydropyridine- 3-carboxylate | MS(ES): 338 (M + 1) for C₁₆H₂₄BNO₆. 1H NMR (300 MHz, FMSO-d6) δ ppm 1.28 (s, 12 H) 3.23 (s, 3 H) 3.56 (t, J = 5.18 Hz, 2 H) 3.74 (s, 3 H) 4.17 (t, J = 5.18 Hz, 2 H) 8.13 (q, J = 2.07 Hz, 2 H) | methyl 5- bromo-1-(2- methoxy- ethyl)-2-oxo- 1,2- dihydro- pyridine-3- carboxylate |
| Intermediate 476-B | 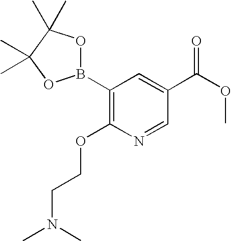 methyl 6-(2-(dimethylamino)- ethoxy)-5-(4,4,5,5-tetramethyl-1,3,2- dioxaborolan-2-yl)nicotinate | MS(ES): 351 (M + 1) for C₁₇H₂₇BN₂O₅. | Intermediate 474 methyl 5- bromo-6-(2- (dimethyl- amino)- ethoxy)- nicotinate |

### Intermediate 477: 2-methoxy-5-{2-[(3-methoxy-5-methylphenyl)amino]-4-[3-(trifluoromethyl)-1H-pyrazol-1-yl]pyrimidin-5-yl}pyridine-3-carbohydrazide

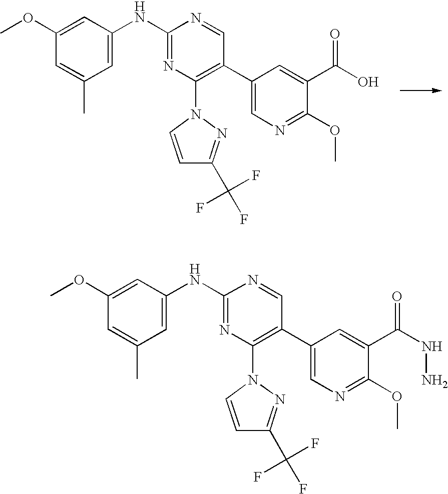

To a solution of 2-methoxy-5-{2-[3-methoxy-5-methylphenyl)amino]-4-[3-(trifluoromethyl)-1H-pyrazol-1-yl]pyrimidin-5-yl}pyridine-3-carboxylic acid (Example 760, 0.15 mmol, 75 mg) in CH₂Cl₂ (10 mL), were added Hydrazine monohydrate (0.38 mmol, 19 mg), triethylamine (0.6 mmol, 0.08 mL), 2-chloro-1-methylpyridinium iodide (0.19 mmol, 48 mg) and 4-(Dimethylamino)pyridine (0.03 mmol, 4 mg) and stirred overnight at RT. Six batches of the same size were run and all the reaction mixtures were combined, diluted with dichloromethane (10 mL) and further washed with 25% citric acid solution (2×10 mL), water (15 mL) and brine (15 mL). The organic layer was dried over Na₂SO₄ and concentrated. The crude mass was purified by flash chromatography (product eluted with 2-3% MeOH in CHCl₃) to afford 300 mg of the title compound.

| Compound | Structure | Mass spectrum | SM |
|---|---|---|---|
| Intermediate 477 | 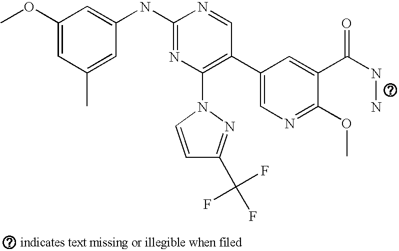 2-methoxy-5-{2-[(3-methoxy-5- methylphenyl)amino]-4-[3- (trifluoromethyl)-1H-pyrazol-1- yl]pyrimidin-5-yl}pyridine-3- carbohydrazide | MS (ES): 515 (M + 1) for C₂₃H₂₁F₃N₈O₃. | Example 760 2-methoxy-5- {2-[(3- methoxy-5- methylphenyl)- amino]-4-[3- (trifluoro methyl)-1H- pyrazol-1- yl]pyrimidin-5- yl}pyridine-3- carboxylic acid |

### Intermediate 478: 2-methoxy-5-{2-[(3-methoxy-5-methylphenyl)amino]-4-[5-methyl-3-(trifluoromethyl)-1H-pyrazol-1-yl]pyrimidin-5-yl]pyridine-3-carbohydrazide

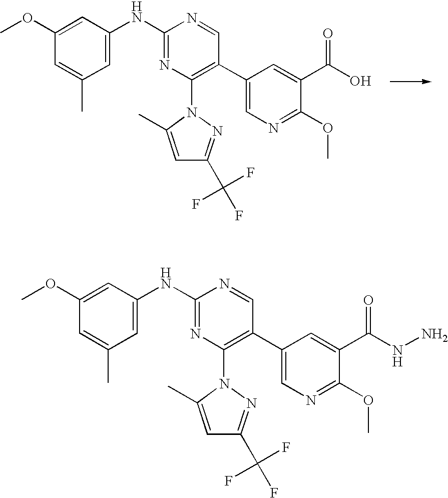

To a solution of 2-methoxy-5-{2-┌(3-methoxy-5-methylphenyl)amino┐-4-┌5-methyl-3-(trifluoromethyl)-1H-pyrazol-1-yl]pyrimidin-5-yl}pyridine-3-carboxylic acid (Example 761, 0.19 mmol, 100 mg) in DMSO (2 mL) was added triethylamine (0.97 mmol, 98 mg) and stirred for 10 min. To this, 2-chloro-1-methyl-pyridinium iodide (0.23 mmol, 60 mg), 4-(dimethylamino)pyridine (0.038 mmol, 5 mg) and hydrazine hydrate (0.48 mmol, 24 mg, 0.023 mL) were added and stirred for 4 h at RT. The reaction mixture was diluted with DCM (25 mL) and further washed with 25% citric acid solution (2×15 mL), water (25 mL) and brine (25 mL). The organic layer was dried over Na₂SO₄ and concentrated. The crude mass was purified by flash chromatography (product eluted with 1-2% MeOH in CHCl₃) to afford the title compound as 85 mg of white solid.

| Compound | Structure | Mass spectrum | SM |
|---|---|---|---|
| Intermediate 478 | 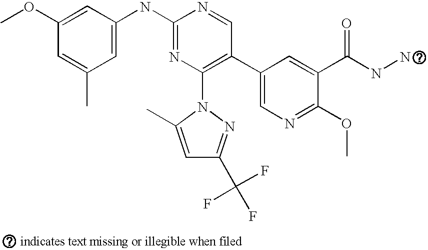 2-methoxy-5-{2-[(3-methoxy-5- methylphenyl)amino]-4-[5-methyl-3- (trifluoromethyl)-1H-pyrazol-1- yl]pyrimidin-5-yl}pyridine-3- carbohydrazide | MS(ES): 529 (M + 1) for C₂₄H₂₃F₃N₈O₃. (66% pure by LCMS). | Example 761 2-methoxy-5-{2- [(3-methoxy-5- methylphenyl)- amino]-4-[5- methyl-3- (trifluoromethyl)- 1H-pyrazol-1- yl]pyrimidin-5- yl}pyridine-3- carboxylic acid |

### Examples

### Example 1

### N²-(3-chloro-4-fluorophenyl)-N⁴-(3-(dimethylamino)propyl)-2′-methoxy-5,5′-bipyrimidine-2,4-diamine

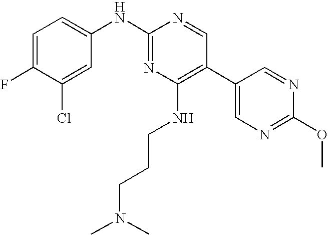

A suspension of 5-bromo-N²-(3-chloro-4-fluoro-phenyl)-N⁴-(3-dimethylamino-propyl)-pyrimidine-2,4-diamine (Intermediate 26, 160 mg, 0.40 mmol), 2-methoxy-5-pyrimidineboronic acid (92 mg, 0.60 mmol), tris(dibenzylideneacetone)dipalladium(0) (36.3 mg, 0.04 mmol), 2-dicyclohexylphosphino-2′,4′,6′-triiso-propyl-1,1′-biphenyl (56.7 mg, 0.12 mmol) and sodium carbonate (42.0 mg, 0.40 mmol) in acetonitrile/water (4 ml:1 ml) was degassed with bubbling nitrogen for 10 min. and then heated to 90° C. After 1 h LC-MS indicated complete reaction, and the reaction mixture was diluted with ethyl acetate (50 mL). The organic layer was separated, dried over sodium sulfate, filtered and concentrated. Flash chromatography (12g column, 0-6% MeOH/CH₂Cl₂ with 0.75% triethylamine) provided 125 mg of the desired product.

MS: ES+ 432 for C₂₀H₂₃ClFN₇O.

1H NMR (300 MHz, DMSO-D6) δ ppm 1.58-1.75 (m, 2H) 2.02 (s, 6H) 2.27 (t, J=6.59 Hz, 2H) 3.34-3.44 (m, 2H) 3.95 (s, 3H) 7.23-7.37 (m, 2H) 7.55-7.67 (m, 1H) 7.74-7.80 (m, 1H) 8.25 (dd, J=6.78, 2.64 Hz, 1H) 8.56 (s, 2H) 9.42 (s, 1H).

The following examples were prepared using the general method described above for Example 1 using 5-bromo-N²-(3-chloro-4-fluoro-phenyl)-N⁴-(3-dimethylamino-propyl)-pyrimidine-2,4-diamine (Intermediate 26) and the starting material (SM) indicated.

| Ex | Compound | Data | SM |
|---|---|---|---|
| 2 | 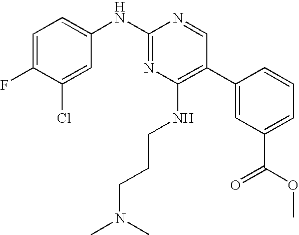 methyl 3-(2-(3-chloro-4-fluorophenylamino)-4- (3-(dimethylamino)propylamino) pyrimidin-5-yl)benzoate | MS: ES+ 458 for C₂₃H₂₅ClFN₅O₂ 1H NMR (300 MHz, DMSO- D6) δ ppm 1.59-1.74 (m, 2 H) 1.93 (s, 6 H) 2.27 (t, J = 6.31 Hz, 2 H) 3.37-3.49 (m, 2 H) 3.86 (s, 3 H) 7.19-7.34 (m, 2 H) 7.56-7.70 (m, 3 H) 7.78 (s, 1 H) 7.85-8.00 (m, 2 H) 8.25 (dd, J = 6.97, 2.64 Hz, 1 H) 9.40 (s, 1 H) | 3-(methoxy carbonyl) phenyl- boronic acid |
| 3 | 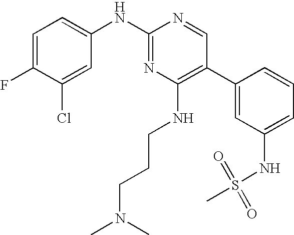 N-(3-(2-(3-chloro-4-fluorophenylamino)-4- (3-(dimethylamino)propylamino) pyrimidin-5-yl)phenyl)methanesulfonamide | MS: ES+ 494 for C₂₂H₂₆ClFN₆O₂S 1H NMR (300 MHz, DMSO- D6) δ ppm 1.84-2.03 (m, 2 H) 2.75 (d, J = 4.71 Hz, 6 H) 2.95- 3.14 (m, 5 H) 3.33-3.49 (m, 2 H) 7.13 (d, J = 7.72 Hz, 1 H) 7.18-7.30 (m, 2 H) 7.37-7.50 (m, 2 H) 7.51-7.75 (m, 2 H) 7.86 (s, 1 H) 8.04 (dd, J = 6.78, 2.45 Hz, 1 H) 9.50 (s, 1 H) 9.95 (s, 1 H) 10.30 (s, 1 H) | 3-(methyl- sulfonamido) phenyl- boronic acid |
| 4 | 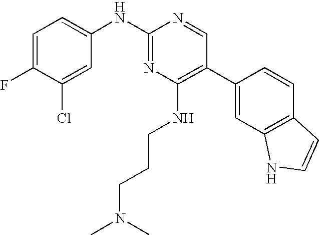 N²-(3-chloro-4-fluorophenyl)-N⁴-(3- (dimethylamino)propyl)-5-(1H-indol-6- yl)pyrimidine-2,4-diamine | MS: ES+ 439 for C₂₃H₂₄ClFN₆ 1H NMR (300 MHz, DMSO- D6) δ ppm 1.60-1.73 (m, 2 H), 1.92 (s, 6 H) 2.29 (t, J = 6.31 Hz, 2 H) 3.38-3.50 (m, 2 H) 6.41- 6.49 (m, 1 H) 6.95 (dd, J = 8.10, 1.51 Hz, 1 H) 7.07 (t, J = 5.09 Hz, 1 H) 7.28 (t, J = 9.14 Hz, 1 H) 7.33-7.39 (m, 2 H) 7.56- 7.68 (m, 2 H) 7.76 (s, 1 H) 8.27 (dd, J = 6.97, 2.64 Hz, 1 H) 9.30 (s, 1 H) 11.17 (s, 1 H) | 1H-indol-6- ylboronic acid |
| 5 | 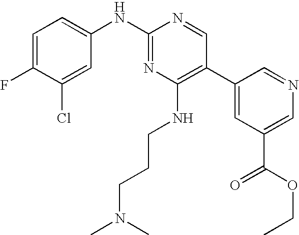 Ethyl 5-(2-(3-chloro-4-fluorophenylamino)-4- (3-(dimethylamino)propylamino) pyrimidin-5-yl)nicotinate | MS: ES+ 473 for C₂₃H₂₆ClFN₆O₂ 1H NMR (300 MHz, DMSO- D6) δ 1.34 (t, J = 7.06 Hz, 3 H) 1.85-2.01 (m, 2 H) 2.75 (d, J = 4.71 Hz, 6 H) 2.96-3.10 (m, 2 H) 3.33-3.49 (m, 2 H) 4.38 (q, J = 7.16 Hz, 2 H) 7.38-7.63 (m, 2 H) 7.91-8.16 (m, 3 H) 8.32 (t, J = 1.98 Hz, 1 H) 8.85 (s, 1 H) 9.14 (s, 1 H) 9.73 (s, 1 H) 10.74 (s, 1 H) | ethyl 5- (4,4,5,5- tetramethyl- 1,3,2- dioxaborolan- 2- yl)nicotinate |
| 6 | 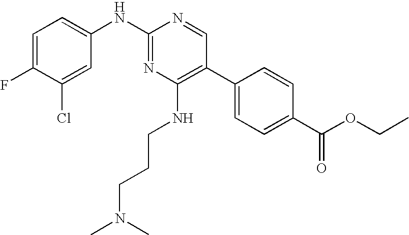 Ethyl 4-(2-(3-chloro-4-fluorophenylamino)-4- (3-(dimethylamino)propylamino) pyrimidin-5-yl)benzoate | MS: ES+ 472 for C₂₄H₂₇ClFN₅O₂ 1H NMR (300 MHz, DMSO- D6) δ ppm 1.32 (t, J = 7.06 Hz, 3 H) 1.60-1.75 (m, 2 H) 1.96 (s, 6 H) 2.28 (t, J = 6.41 Hz, 2 H) 3.37-3.52 (m, 2 H) 4.33 (q, J = 7.10 Hz, 2 H) 7.22-7.36 (m, 2 H) 7.52 (d, J = 8.48 Hz, 2 H) 7.57-7.68 (m, 1 H) 7.83 (s, 1 H) 8.01 (d, J = 8.48 Hz, 2 H) 8.25 (dd, J = 6.97, 2.64 Hz, 1 H) 9.44 (s, 1 H) | 4-(ethoxy carbonyl) phenyl boronic acid |
| 7 | 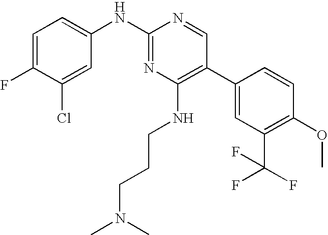 N²-(3-chloro-4-fluorophenyl)-N⁴-(3- (dimethylamino)propyl)-5-(4-methoxy- 3(trifluoromethyl)phenyl)pyrimidine-2,4- diamine | MS: ES+ 498 for C₂₃H₂₄ClF₄N₅O 1H NMR (300 MHz, DMSO- D6) δ ppm 1.91 (m, 2 H) 2.74 (d, J = 4.71 Hz, 6 H) 2.95-3.11 (m, 2 H) 3.33-3.47 (m, 2 H) 3.94 (s, 3 H) 7.43 (dd, J = 20.25, 8.76 Hz, 2 H) 7.50-7.59 (m, 1 H) 7.59-7.81 (m, 3 H) 7.87 (s, 1 H) 8.02 (dd, J = 6.78, 2.26 Hz, 1 H) 9.37-9.59 (m, 1 H) 10.35 (s, 1 H) | 4-methoxy-3- (trifluoro methyl)- phenyl- boronic acid |
| 8 | 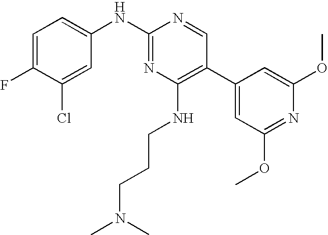 N²-(3-chloro-4-fluorophenyl)-5-(2,6- dimethoxypyridin-4-yl)-N⁴-(3- (dimethylamino)propyl)-pyrimidin-2,4- diamine | MS: ES+ 461 for C₂₂H₂₆ClFN₆O₂ 1H NMR (300 MHz, DMSO- D6) δ ppm 1.58-1.70 (m, 2 H) 1.98 (s, 6 H) 2.25 (t, J = 6.50 Hz, 2 H) 3.34-3.44 (m, 2 H) 3.76- 3.94 (m, 6 H) 6.36-6.49 (m, 1 H) 6.82 (t, J = 5.18 Hz, 1 H) 7.26 (t, J = 9.14 Hz, 1 H) 7.43-7.55 (m, 1 H) 7.56-7.66 (m, 2 H) 8.26 (dd, J = 6.88, 2.54 Hz, 1 H) 9.27 (s, 1 H) | 2,6- dimethoxy pyridin-4-yl boronic acid |
| 9 | 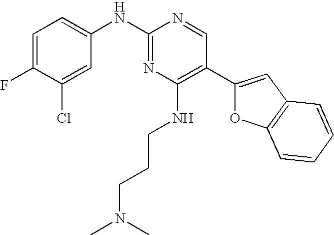 5-(benzofuran-2-yl)-N²-(3-chloro-4- fluorophenyl)-N⁴-(3-(dimethylamino) propyl)pyrimidine-2,4-diamine | MS: ES+ 440 for C₂₃H₂₃ClFN₅O 1H NMR (300 MHz, DMSO- D6) δ 1.69-1.82 (m, 2 H) 2.08 (s, 6 H) 2.35 (t, J = 6.59 Hz, 2 H) 3.49-3.62 (m, 2 H) 7.08 (d, J = 0.75 Hz, 1 H) 7.21-7.45 (m, 3 H) 7.49-7.66 (m, 4 H) 8.24 (dd, J = 6.97, 2.64 Hz, 1 H) 8.35 (s, 1 H) 9.64 (s, 1 H) | benzofuran- 2-ylboronic acid |
| 10 | 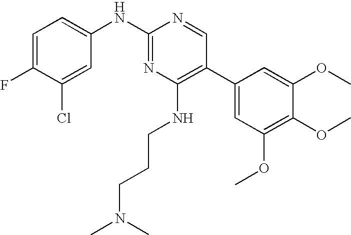 N²-(3-chloro-4-fluorophenyl)-N⁴-(3- (dimethylamino)propyl)-5-(3,4,5- trimethoxyphenyl)pyrimidin-2,4-diamine | MSP: ES+ 490 for C₂₄H₂₉ClFN₅O₃ 1H NMR (300 MHz, DMSO- D6) δ ppm 1.61-1.75 (m, 2 H) 1.99 (s, 6 H) 2.28 (t, J = 6.69 Hz, 2 H) 3.42 (q, J = 6.15 Hz, 2 H) 3.68 (s, 3 H) 3.80 (s, 6 H) 6.62 (s, 2 H) 7.12 (s, 1 H) 7.27 (t, J = 9.14 Hz, 1 H) 7.47-7.65 (m, 1 H) 7.79 (s, 1 H) 8.27 (dd, J = 6.97, 2.64 Hz, 1 H) 9.33 (s, 1 H) | 3,4,5- trimethoxy phenyl- boronic acid |
| 11 | 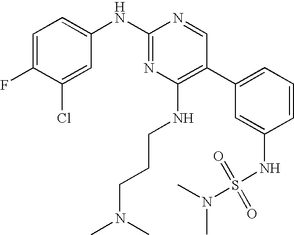 N′-[3-(2-[(3-chloro-4-fluorophenyl)amino]-4- {[3-(dimethylamino)propyl]amino} pyrimidin-5-yl)phenyl]-N,N- dimethylsulfonamide | MS: ES+ 522 for C₂₃H₂₉ClFN₇O₂S 1H NMR (300 MHz, DMSO- D6) δ 1.88-2.03 (m, 2 H) 2.72 (s, 12 H) 2.98-3.12 (m, 2 H) 3.39-3.52 (m, 2 H) 6.64- 6.79 (m, 1 H) 7.07 (d, J = 7.54 Hz, 1 H) 7.12-7.23 (m, 1 H) 7.25-7.44 (m, 2 H) 7.56-7.68 (m, 1 H) 7.75 (s, 1 H) 8.18 (dd, J = 6.78, 2.45 Hz, 1 H) 9.44 (s, 1 H) 9.79 (s, 1 H) 10.03 (s, 1 H) | 3-(N,N- dimethyl sulfamoyl amino)phenyl boronic acid |
| 12 | 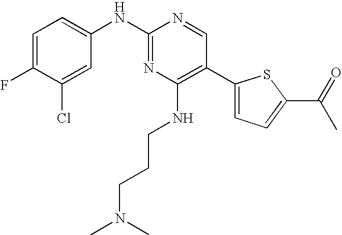 1-{5-[2-(3-chloro-4-fluorophenylamino)-4- [3-(dimethylamino)propylamino) pyrimidin-5-yl]thiophen-2-yl}ethanone | MS: ES+ 449 for C₂₁H₂₃ClFN₅OS 1H NMR (300 MHz, DMSO- D6) δ ppm 1.64-1.78 (m, Hz, 2 H) 2.04 (s, 6 H) 2.32 (t, J = 6.31 Hz, 2 H) 2.54 (s, 3 H) 3.40- 3.53 (m, 2 H) 7.20-7.36 (m, 2 H) 7.53-7.67 (m, 2 H) 7.95- 8.04 (m, 2 H) 8.21 (dd, J = 6.97, 2.64 Hz, 1 H) 9.58 (s, 1 H) | 5-acetyl thiophen-2-yl boronic acid |
| 13 | 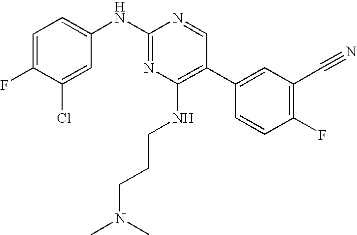 5-{2-(3-chloro-4-fluorophenylamino)-4- [3-(dimethylamino)propyl amino]pyrimidin-5-yl}-2-fluorobenzonitrile | MS: ES+ 443 for C₂₂H₂₁ClF₂N₆ | 3-cyano-4- fluorophenyl boronic acid |
| 14 | 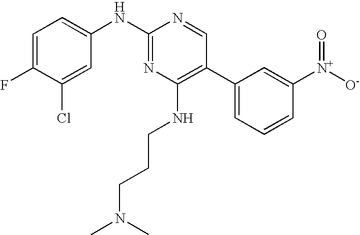 N²-(3-chloro-4-fluorophenyl)-N⁴-[3- (dimethylamino)propyl]-5-(3-nitrophenyl) pyrimidin-2,4-diamine | MS: ES+ 445 for C₂₁H₂₂ClFN₆O₂ 1H NMR (300 MHz, DMSO- D6) δ 1.59-1.75 (m, Hz, 2 H) 1.98 (s, 6 H) 2.28 (t, J = 6.50 Hz, 2 H) 3.37-3.51 (m, 2 H) 7.24-7.36 (m, 2 H) 7.57-7.67 (m, 1 H) 7.74 (t, J = 7.82 Hz, 1 H) 7.80-7.91 (m, 2 H) 8.13- 8.33 (m, 3 H) 9.47 (s, 1 H) | 3-nitrophenyl boronic acid |
| 15 | 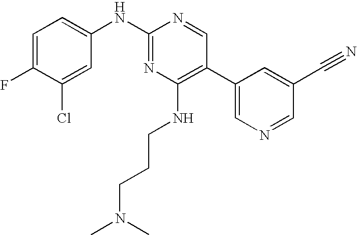 5-{2-(3-chloro-4-fluorophenylamino)-4- [3-(dimethylamino)propylamino] pyrimidin-5-yl}nicotinitrile | MS: ES+ 426 for C₂₁H₂₁ClFN₇ 1H NMR (300 MHz, DMSO- D6) δ ppm 1.85-2.02 (m, 2 H) 2.75 (d, J = 4.33 Hz, 6 H) 3.05 (dd, J = 9.70, 4.80 Hz, 2 H) 3.41 (t, J = 5.97 Hz, 2 H) 7.44 (t, J = 9.04 Hz, 1 H) 7.52-7.63 (m, 1 H) 7.87-8.08 (m, 3 H) 8.39 (t, J = 1.98 Hz, 1 H) 8.89 (d, J = 2.07 Hz, 1 H) 9.07 (d, J = 1.88 Hz, 1 H) 9.88 (s, 1 H) 10.61 (s, 1 H) | 5-cyano pyridin-3-yl boronic acid |
| 16 | 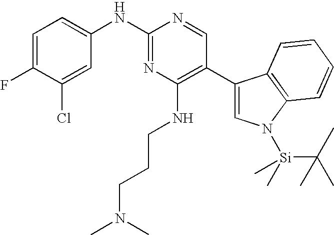 5-[1-(tert-butyldimethylsilyl)-1H- indol-3-yl]-N²-(3-chloro-4-fluorophenyl)- N⁴-[3-(dimethylamino)propyl] pyrimidin-2,4-diamine | MS: ES+ 553 for C₂₉H₃₈ClFN₆Si 1H NMR (300 MHz, DMSO- D6) δ ppm 0.64 (s, 6 H) 0.93 (s, 9 H) 1.86-2.02 (m, 2 H) 2.74 (d, J = 4.71 Hz, 6 H) 2.93-3.12 (m, 2 H) 3.32-3.50 (m, 2 H) 7.08-7.28 (m, 2 H) 7.43-7.51 (m, 1 H) 7.51-7.60 (m, 2 H) 7.63 (d, J = 8.29 Hz, 1 H) 7.87- 7.97 (m, 2 H) 8.01 (dd, J = 6.69, 2.35 Hz, 1 H) 9.68 (s, 1 H) 10.79 (s, 1 H) | 1-(tert- butyldimethyl- silyl)-1H- indol-3-yl boronic acid |
| 17 | 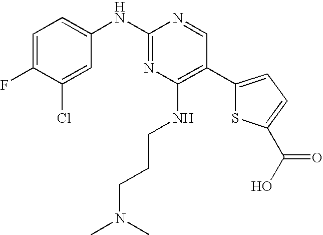 5-{2-(3-chloro-4-fluorophenylamino)-4- [3-(dimethylamino)propylamino] pyrimidin-5-yl}thiophene-2-carboxylic acid | MS: ES+ 450 for C₂₀H₂₁ClFN5O₂S 1H NMR (300 MHz, DMSO- D6) δ ppm 1.89-2.04 (m, 2 H) 2.75 (d, J = 4.71 Hz, 6 H) 3.00- 3.14 (m, 2 H) 3.38-3.51 (m, 2 H) 7.26 (d, J = 3.77 Hz, 1 H) 7.33-7.48 (m, 2 H) 7.53-7.64 (m, 1 H) 7.76 (d, J = 3.77 Hz, 1 H) 8.03 (s, 1 H) 8.10 (dd, J = 6.88, 2.54 Hz, 1 H) 9.50 (s, 1 H) 9.96 (s, 1 H) | 5-borono- thiophene-2- carboxylic acid |
| 18 | 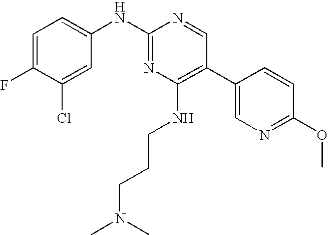 N²-(3-chloro-4-fluorophenyl)-N⁴-[3- (dimethylamino)propyl]-5-(6-methoxy- pyridin-3-yl)pyrimidin-2,4-diamine | MS: ES+ 431 for C₂₁H₂₄ClFN₆O 1H NMR (300 MHz, DMSO- D6) δ ppm 1.84-1.99 (m, 2 H) 2.74 (d, J = 4.71 Hz, 6 H) 2.94- 3.09 (m, 2 H) 3.32-3.46 (m, 2 H) 3.90 (s, 3 H) 6.95 (d, J = 8.67 Hz, 1 H) 7.40-7.59 (m, 2 H) 7.73 (dd, J = 8.67, 2.45 Hz, 1 H) 7.81-8.05 (m, 3 H) 8.20 (d, J = 2.26 Hz, 1 H) 9.66 (s, 1 H) 10.63 (s, 1 H) | 2-methoxy-5- (4,4,5,5- tetramethyl- 1,3,2- dioxaborolan- 2-yl) pyridine |
| 19 | 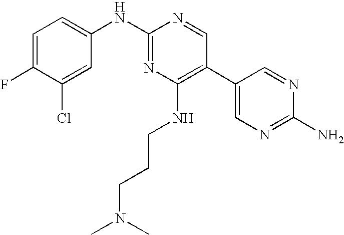 N²-(3-chloro-4-fluorophenyl)-N⁴-[3- (dimethylamino)propyl]-5,5′- bipyrimidin-2,2′,4-triamine | MS: ES+ 417 for C₁₉H₂₂ClFN₈ 1H NMR (300 MHz, DMSO- D6) δ ppm 1.82-1.99 (m, 2 H) 2.74 (d, J = 2.83 Hz, 6 H) 2.93- 3.15 (m, 2 H) 3.30-3.46 (m, 2 H) 6.87 (s, 1 H) 7.25-7.49 (m, 2 H) 7.52-7.62 (m, 1 H) 7.78 (s, 1 H) 8.06-8.16 (m, 2 H) 8.21 (s, 2 H) 9.64 (s, 1 H) 9.87 (s, 1 H) | 2-amino pyrimidin-5- yl boronic acid |
| 20 | 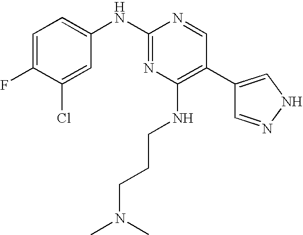 N²-(3-chloro-4-fluorophenyl)-N⁴- [3-(dimethylamino)propyl]-5-(1H- pyrazol-4-yl)pyrimidine-2,4-diamine | MS: ES+ 390 for C₁₈H₂₁ClFN₇ 1H NMR (300 MHz, DMSO-d₆) δ ppm 1.92 (m, 2 H) 2.75 (d, J = 4.52 Hz, 6 H) 2.90-3.10 (m, 2 H) 3.36-3.50 (m, 2 H) 7.40- 7.59 (m, 2 H) 7.76-7.94 (m, 3 H) 7.99 (dd, J = 6.78, 2.45 Hz, 1 H) 9.57 (br. s., 1 H) 10.18 (br. s., 1 H) | 4-(4,4,5,5- tetramethyl- 1,3,2- dioxaborolan- 2-yl)-1H- pyrazole |
| 21 | 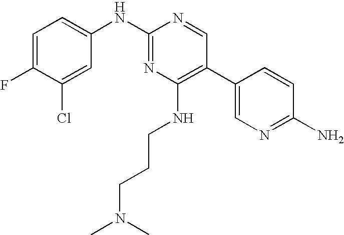 5-(6-aminopyridin-3-yl)-N²-(3-chloro-4- fluorophenyl)-N⁴-[3-(dimethylamino) propyl]pyrimidine-2,4-diamine | MS: ES+ 416 for C₂₀H₂₃ClFN₇ 1H NMR (300 MHz, DMSO-d₆) δ ppm 1.86-2.01 (m, 2 H) 2.75 (d, J = 3.96 Hz, 6 H) 2.99-3.12 (m, 2 H) 3.32-3.49 (m, 2 H) 7.09 (d, J = 9.04 Hz, 1 H) 7.40- 7.59 (m, 2 H) 7.80-8.00 (m, 3 H) 8.05 (d, J = 1.51 Hz, 1 H) 8.36 (br. s., 1 H) 8.54 (br. s., 2 H) 10.12 (br. s., 1 H) 11.15 (br. s., 1 H) | 5-(4,4,5,5- tetramethyl- 1,3,2- dioxaborolan- 2-yl)pyridin- 2-amine |
| 22 | 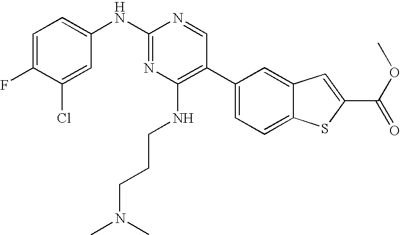 Methyl 5-{2-(3-chloro-4-fluorophenylamino)- 4-[3-(dimethylamino)propylamino] pyrimidin-5-yl}benzo[b]thiophene-2- carboxylate | MS: ES+ 514 for C₂₅H₂₅ClFN₅O₂S 1H NMR (300 MHz, DMSO-d₆) δ ppm 1.85-1.99 (m, 2 H) 2.75 (d, J = 4.71 Hz, 6 H) 2.97-3.08 (m, 2 H) 3.36-3.46 (m, 2 H) 3.90 (s, 3 H) 7.44 (t, J = 9.04 Hz, 1 H) 7.52-7.72 (m, 3 H) 7.92 (s, 1 H) 8.02-8.10 (m, 2 H) 8.21 (d, J = 8.48 Hz, 1 H) 8.27 (s, 1 H) 9.51 (br. s., 1 H) 10.26 (br. s., 1 H) | Methyl 5- (4,4,5,5- tetramethyl- 1,3,2- dioxaborolan- 2-yl)-1- benzo thiophene-2- carboxylate |
| 23 | 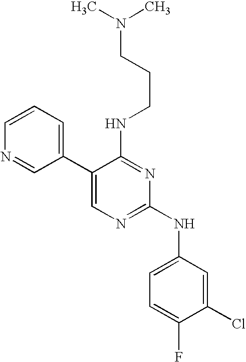 N²-(3-chloro-4-fluorophenyl)-N⁴-[3- (dimethylamino)propyl]-5-pyridin-3- ylpyrimidin-2,4-diamine | MS (ES) (M + H)⁺ 401 for C₂₀H₂₂ClFN₆ ¹H-NMR (DMSO-d₆) δ: 11.04 (s, 1 H), 10.66 (s, 1 H), 8.95 (s, 1 H), 8.89 (d, J = 4.71 Hz, 1 H), 8.55 (s, 1 H), 8.39 (d, J = 6.97 Hz, 1 H), 8.02-8.07 (m, 1 H), 7.90-8.02 (m, J = 11.87 Hz, 2 H), 7.44-7.64 (m, 2 H), 3.26- 3.53 (m, 2 H), 2.92-3.13 (m, 2 H), 2.69 (s, J = 3.00 Hz, 6 H), 1.83-2.06 (m, J = 5.65 Hz, 2 H). ¹⁹F NMR (DMSO-d6) δ: 121.61 (s, 1 F). | 3-pyridyl boronic acid |
| 24 | 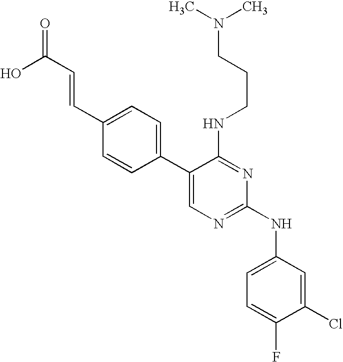 (2E)-3-[4-(2-[(3-chloro-4- fluorophenyl)amino]-4-{[3- (dimethylamino)propyl]-amino}pyrimidin-5- yl)phenyl]prop-2-enoic acid | MS (ES) (M + H)⁺ 470 for C₂₄H₂₆ClFN₅O₂ MS (ES) (M − H)⁻ 468 for C₂₄H₂₄ClFN₅O₂ ¹H-NMR (DMSO-d₆) δ: 9.36 (s, 1 H), 8.25 (dd, J = 6.88, 2.54 Hz, 1 H), 7.77 (s, 1 H), 7.60-7.68 (m, J = 9.23 Hz, 1 H), 7.57 (d, J = 8.29 Hz, 2 H), 7.32 (d, J = 8.29 Hz, 2 H), 7.22-7.30 (m, 3 H), 7.16 (d, J = 15.82 Hz, 1 H), 6.42 (d, J = 15.82 Hz, 1 H), 3.43 (td, J = 6.78, 5.09 Hz, 2H), 2.28 (t, J = 5.65 Hz, 2 H), 1.96 (s, 6 H), 1.66 (tt, J = 7.35, 6.78, 6.22, 5.09 Hz, 2 H). ¹⁹F NMR (DMSO-d6) δ: 127.21 (s, 1 F). | (E)-3-(4- boronophenyl)- prop-2-enoic acid |
| 25 | 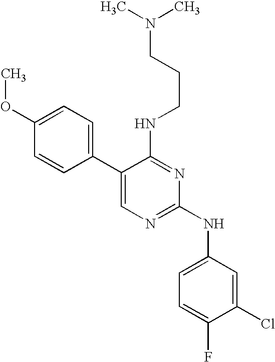 N²-(3-chloro-4-fluorophenyl)-N⁴-[3- (dimethylamino)propyl]-5-(4- methoxyphenyl)-pyrimidine-2,4-diamine | MS (ES) (M + H)⁺ 430 for C₂₂H₂₅ClFN₅O ¹H-NMR (DMSO-d₆) δ: 9.30 (s, 1 H), 8.25 (dd, J = 6.78, 2.64 Hz, 1 H), 7.70 (s, 1 H), 7.61 (td, J = 6.45, 2.73 Hz, 1 H), 7.20- 7.34 (m, 3 H), 7.09 (t, J = 4.52 Hz, 1 H), 7.02 (d, J = 8.67 Hz, 2 H), 3.78 (s, 3 H), 3.42 (q, J = 5.78 Hz, 2 H), 2.28 (t, J = 5.93 Hz, 2 H), 1.98 (s, 6 H), 1.66 (ddd, J = 12.39, 6.08, 5.84 Hz, 2 H). ¹⁹F NMR (DMSO-d6) δ: 127.37 (s, 1 F). | (4- methoxy- phenyl)boronic acid |
| 26 | 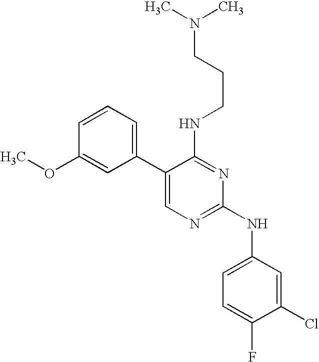 N²-(3-chloro-4-fluorophenyl)-N⁴-[3- (dimethylamino)propyl]-5-(3- methoxyphenyl)-pyrimidine-2,4-diamine | MS (ES) (M + H)⁺ 430 for C₂₂H₂₅ClFN₅O ¹H-NMR (DMSO-d6) δ: 9.34 (s, 1 H), 8.25 (dd, J = 6.88, 2.35 Hz, 1 H), 7.77 (s, 1 H), 7.56-7.66 (m, 1 H), 7.37 (t, J = 7.72 Hz, 1 H), 7.28 (t, J = 9.14 Hz, 1 H), 7.20 (t, J = 5.27 Hz, 1 H), 6.87- 6.96 (m, 3 H), 3.78 (s, 3 H), 3.43 (q, J = 6.41 Hz, 2 H), 2.28 (t, J = 6.97, 6.41 Hz, 2 H), 1.97 (s, 6 H), 1.67 (tt, J = 6.59, 6.03 Hz, 2 H). ¹⁹F MR (DMSO-d6) δ: 127.22 (s, 1 F). | (3- methoxy- phenyl)boronic acid |
| 27 | 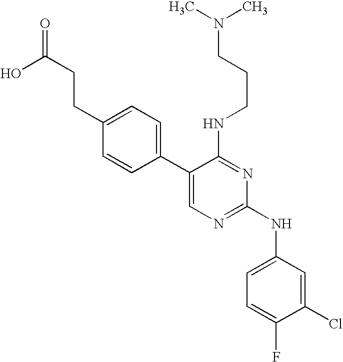 3-[4-(2-[(3-chloro-4-fluorophenyl)amino]-4- {[3-(dimethylamino)propyl]- amino}pyrimidin-5-yl)phenyl]propanoic acid | MS (ES) (M + H)⁺ 472 for C₂₄H₂₈ClFN₅O₂ MS (ES) (M − H)⁻ 470 for C₂₄H₂₆ClFN₅O₂ ¹H-NMR (DMSO-d₆) δ: 9.31 (s, 1 H), 8.24 (dd, J = 6.78, 1.88 Hz, 1 H), 7.71 (s, 1 H), 7.53-7.66 (m, 1 H), 7.25-7.33 (m, 2 H), 7.15-7.25 (m, 4 H), 3.42 (q, J = 5.71 Hz, 2 H), 2.79 (t, J = 7.72 Hz, 2 H), 2.27 (t, J = 6.78, 5.65 Hz, 2 H), 2.20 (t, J = 8.67, 7.54 Hz, 2 H), 1.95 (s, 6 H), 1.65 (tt, J = 6.03 Hz, 2 H). ¹⁹F NMR (DMSO-d6) δ: 127.35 (s, 1 F). | 3-(4- boronophenyl)- propanoic acid |
| 28 | 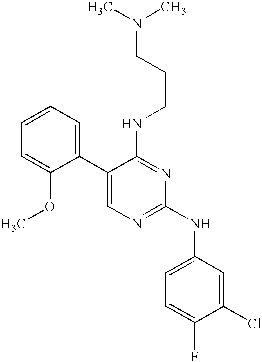 N²-(3-chloro-4-fluorophenyl)-N⁴-[3- (dimethylamino)propyl]-5-(2- methoxyphenyl)-pyrimidin-2,4-diamine | MS (ES) (M + H)⁺ 430 for C₂₂H₂₅ClFN₅O MS (ES) (M − H)⁻ 428 for C₂₂H₂₃ClFN₅O ¹H-NMR (DMSO-d₆) δ: 9.75 (s, 1 H), 8.01 (br. s., 1 H), 7.73 (s, 1 H), 7.51-7.60 (m, 1 H), 7.40- 7.52 (m, 2 H), 7.20-7.26 (m, 1H), 7.15 (d, J = 8.67 Hz, 1 H), 7.06 (t, J = 7.35 Hz, 1 H), 3.78 (s, 3 H), 3.40 (d, J = 6.22 Hz, 2 H), 2.90-3.05 (m, 2 H), 2.72 (d, J = 4.71 Hz, 6 H), 1.81-2.02 (m, 2 H). | (2- methoxy- phenyl)boronic acid |
| 29 | 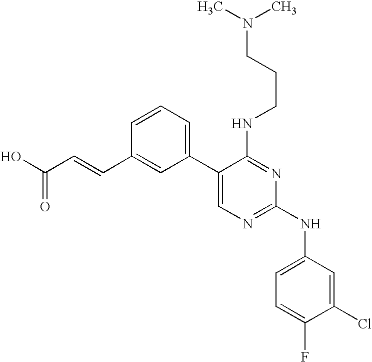 (2E)-3-[3-(2-[(3-chloro-4- fluorophenyl)amino]-4-{[3- (dimethylamino)propyl]-amino}pyrimidin-5- yl)phenyl]prop-2-enoic acid | MS (ES) (M + H)⁺ 470 for C₂₄H₂₆ClFN₅O₂ MS (ES) (M − H)⁻ 468 for C₂₄H₂₄ClFN₅O₂ ¹H-NMR (DMSO-d₆) δ: 9.37 (s, 1 H), 8.25 (dd, J = 6.88, 2.54 Hz, 1 H), 7.78 (s, 1 H), 7.54-7.68 (m, 3 H), 7.41-7.52 (m, 2 H), 7.19-7.40 (m, 4 H), 6.55 (d, J = 16.20 Hz, 1 H), 3.43 (q, J = 5.65 Hz, 2 H), 2.28 (t, J = 6.22, 5.09 Hz, 2 H), 1.95 (s, 6 H), 1.59-1.73 (m, 2 H). ¹⁹F NMR (DMSO-d6) δ: 127.19 (s, 1 F). | (E)-3-(3- boronophenyl)- prop-2-enoic acid |
| 30 | 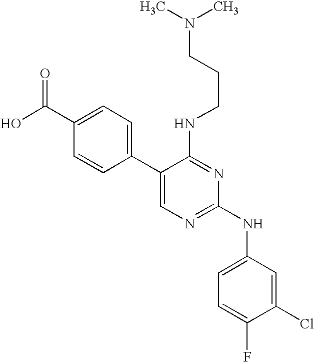 4-(2-[(3-chloro-4-fluorophenyl)amino]-4- {[3-(dimethylamino)propyl]- amino}pyrimidin-5-yl)benzoic acid | MS (ES) (M + H)⁺ 444 for C₂₂H₂₄ClFN₅O₂ MS (ES) (M − H)⁻ 442 for C₂₂H₂₂ClFN₅O₂ ¹H-NMR (DMSO-d₆) δ: 9.38 (s, 1 H), 8.24 (dd, J = 6.97, 2.45 Hz, 1 H), 7.95 (d, J = 8.10 Hz, 2 H), 7.78 (s, 1 H), 7.54-7.68 (m, 1 H), 7.35 (d, J = 7.91 Hz, 2 H), 7.20-7.32 (m, J = 9.04, 9.04 Hz, 2 H), 3.43 (td, J = 6.41, 5.27 Hz, 2 H), 2.29 (t, J = 6.41 Hz, 2 H), 1.96 (s, 6 H), 1.67 (tt, J = 6.22 Hz, 2 H). ¹⁹F NMR (DMSO-d6) δ: 127.16 (s, 1 F). | 4-borono- benzoic acid |
| 31 | 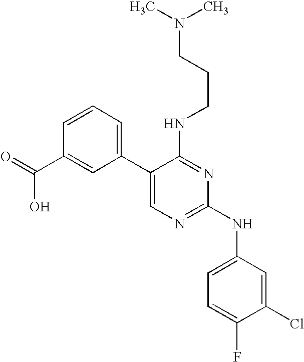 3-(2-[(3-chloro-4-fluorophenyl)amino]-4- {[3-(dimethylamino)propyl]- amino}pyrimidin-5-yl)benzoic acid | MS (ES) (M + H)⁺ 444 for C₂₂H₂₄ClFN₅O₂ MS (ES) (M − H)⁻ 442 for C₂₂H₂₂ClFN₅O₂ ¹H-NMR (DMSO-d₆) δ: 9.33 (s, 1 H), 8.24 (dd, J = 7.06, 2.35 Hz, 1 H), 7.78-7.92 (m, 2 H), 7.71 (s, 1 H), 7.56-7.68 (m, 1 H), 7.36 (t, J = 7.54 Hz, 1 H), 7.22- 7.32 (m, 2 H), 7.18 (t, J = 4.90 Hz, 1 H), 3.43 (td, J = 6.59, 5.09 Hz, 2 H), 2.26 (t, J = 5.75 Hz, 2 H), 1.91 (s, 6 H), 1.64 (dt, J = 12.57, 6.43 Hz, 2 H). ¹⁹F NMR (DMSO-d6) δ: 127.36 (s, 1 F). | 3-borono- benzoic acid |
| 32 | 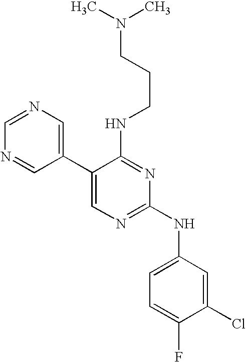 N²-(3-chloro-4-fluorophenyl)-N⁴-[3- (dimethylamino)propyl]-5,5′-bipyrimidine-2,4- diamine | MS (ES) (M + H)⁺ 402 for C₁₉H₂₂ClFN₇ ¹H-NMR (DMSO-d₆) δ: 9.50 (s, 1 H), 9.17 (s, 1 H), 8.81 (s, 2 H), 8.24 (dd, J = 6.88, 2.54 Hz, 1 H), 7.86 (s, 1 H), 7.62 (ddd, J = 9.00, 4.10, 2.73 Hz, 1 H), 7.45 (t, J = 4.33 Hz, 1 H), 7.30 (t, J = 9.14 Hz, 1 H), 3.41 (dt, J = 6.41, 5.27 Hz, 2 H), 2.42 (t, J = 5.56 Hz, 2 H), 2.14 (s, 6 H), 1.73 (tt, J = 6.59 Hz, 2 H). ¹⁹F NMR (DMSO-d6) δ: 126.69 (s, 1 F). | pyrimidin-5- ylboronic acid |

The following examples were prepared using the general method described above for Example 1 using pyrimidin-5-ylboronic acid and the starting material (SM) indicated.

| Ex | Compound | Data | SM |
|---|---|---|---|
| 33 | 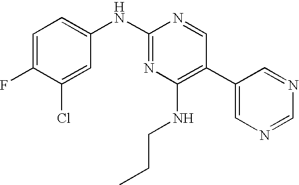 N²-(3-chloro-4- fluorophenyl)-N⁴- propyl-5-pyrimidin- 5-ylpyrimidine-2,4- diamine | MS(ES): 359 (M + 1) for C₁₇H₁₆ClFN₆ ¹H NMR (400 MHz, DMSO-d₆): δ 0.86-0.91 (m, 3H), 1.23 (d, J = 6.92 Hz, 2H), 1.58 (q, J = 7.44 Hz, 2H), 7.12 (t, J = 5.68 Hz, 1H), 7.29 (t, J = 9.12 Hz, 1H), 7.55- 7.59 (m, 1H), 7.82 (s, 1H), 8.28 (dd, J = 6.9, 2.56 Hz, 1H), 8.78 (s, 2H), 9.15 (s, 1H), 9.48 (s, 1H). | 5-Bromo-N²- (3-chloro-4- fluoro- phenyl)-N⁴- propyl- pyrimidine- 2,4-diamine (Intermediate 35) |
| 34 | 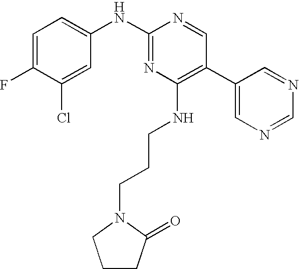 1-[3-[[2-[(3-chloro-4- fluorophenyl)amino]- 5-pyrimidin-5- ylpyrimidin-4- yl]amino]propyl] pyrrolidin-2-one | MS(ES): 442 (M + 1) for C₂₁H₂₁ClFN₇O. ¹H NMR (400 MHz, DMSO-d₆): δ 1.72-1.77 (m, 2H), 1.85-1.89 (m, 2H), 2.16 (t, J = 8.2 Hz, 2H), 3.15 (s, 1H), 3.22 (t, J = 7.04 Hz, 2H), 3.28 (m, 3H), 7.07 (br s, 1H), 7.32 (t, J = 9.16 Hz, 1H), 7.6-7.64 (m, 1H), 7.85 (s, 1H), 8.19 (dd, J = 6.86, 2.48 Hz, H), 8.82 (s, 2H), 9.16 (s, 1H), 9.53 (s, 1H). | 1-{3-[5- Bromo-2-(3- chloro-4- fluoro- phenylamino)- pyrimidin-4- ylamino]- propyl}- pyrrolidin-2- one (Intermediate 36) |
| 35 | 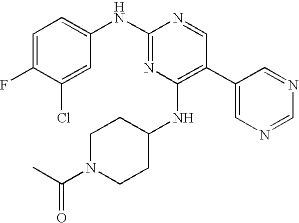 1-[4-[[2-[(3-chloro-4- fluorophenyl)amino]- 5-pyrimidin-5- ylpyrimidin-4- yl]amino]piperidin-1- yl]ethanone | MS(ES): 442 (M + 1) for C₂₁H₂₁ClFN₇O. ¹H NMR (400 MHz, DMSO-d₆): δ 1.36-1.46 (m, 2H), 1.84-1.93 (m, 2H), 1.98 (s, 3H), 2.64 (t, J = 10.56 Hz, 1H), 3.12 (t, J = 11.40 Hz, 1H), 3.85 (d, J = 13.48 Hz, 1H), 4.20 (m, 1H), 4.36 (d, J = 13.68 Hz, 1H), 6.84 (d, J = 7.72 Hz, 1H), 7.33 (t, J = 9.08 Hz, 1H), 7.56 (m, 1H), 7.86 (s, 1H), 8.23 (dd, J = 6.90, 2.04 Hz, 1H), 8.79 (s, 2H), 9.15 (s, 1H), 9.54 (s, 1H). | 1-{4-[5- Bromo-2-(3- chloro-4- fluoro- phenylamino)- pyrimidin-4- ylamino]- piperidin-1- yl}-ethanone (Intermediate 72) |
| 36 | 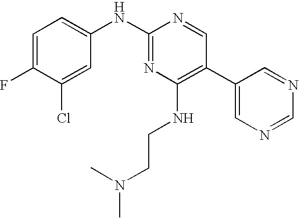 N²-(3-chloro-4- fluorophenyl)-N⁴-(2- dimethylaminoethyl)- 5-pyrimidin-5- ylpyrimidine-2,4- diamine | MS(ES): 388 (M + 1) for C₁₈H₁₉ClFN₇. ¹H NMR (400 MHz, DMSO-d₆): δ 2.31 (br s, 6H), 2.65 (br s, 2H), 3.49-3.51 (br s, 2H), 6.98 (br s, 1H), 7.3 (t, J = 9.1 Hz, 1H), 7.64 (ddd, J = 9.02, 4.12, 2.72 Hz, 1H), 7.87 (s, 1H), 8.15 (dd, J = 6.88, 2.5 Hz, 1H), 8.81 (s, 2H), 9.16 (s, 1H), 9.49 (s, 1H). | 5-Bromo-N²- (3-chloro-4- fluoro- phenyl)-N⁴- (2- dimethylamino- ethyl)- pyrimidine- 2,4-diamine (Intermediate 37) |
| 37 | 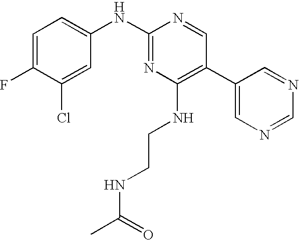 N-[2-[[2-[(3-chloro- 4- fluorophenyl)amino]- 5-pyrimidin-5- ylpyrimidin-4- yl]amino]ethyl] acetamide | MS(ES): 402 (M + 1) for C₁₈H₁₇ClFN₇O ¹H NMR (400 MHz, MeOD): δ 1.9 (s, 3H), 3.43 (t, J = 5.52 Hz, 2H), 3.57 (q, J = 5.33 Hz, 2H), 7.16 (t, J = 9 Hz, 1H), 7.5 (ddd, J = 9, 4.1, 2.68 Hz, 1H), 7.82 (s, 1H), 8.05 (dd, J = 6.74, 2.68 Hz, 1H), 8.85 (s, 2H), 9.14 (s, 1H). | N-{2-[5- Bromo-2-(3- chloro-4- fluoro- phenylamino)- pyrimidin-4- ylamino]- ethyl}- acetamide (Intermediate 38) |
| 38 | 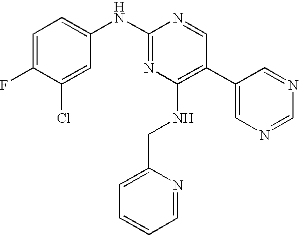 N²-(3-chloro-4- fluorophenyl)-N⁴- (pyridin-2-ylmethyl)- 5-pyrimidin-5- ylpyrimidine-2,4- diamine | MS(ES): 408 (M + 1) for C₂₀H₁₅ClFN₇ ¹H NMR (400 MHz, DMSO-d₆): δ 4.66 (d, J = 5.96 Hz, 2H), 7.15 (t, J = 9.24 Hz, 1H), 7.21-7.24 (m, 1H), 7.35 (d, J = 7.84 Hz, 1H), 7.48-7.52 (m, 1H), 7.66-7.74 (m, 2H), 7.92 (m, 2H), 8.52 (d, J = 4.8 Hz, 1H), 8.91 (s, 2H), 9.19 (s, 1H), 9.45 (s, 1H). | 5-Bromo-N²- (3-chloro-4- fluoro- phenyl)-N⁴- pyridin-2- ylmethyl- pyrimidine- 2,4-diamine (Intermediate 39) |
| 39 | 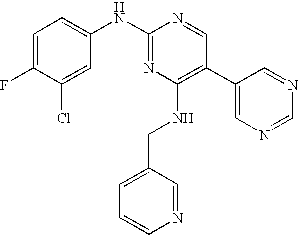 N²-(3-chloro-4- fluorophenyl)-N⁴- (pyridin-3-ylmethyl)- 5-pyrimidin-5- ylpyrimidine-2,4- diamine | MS(ES): 408 (M + 1) for C₂₀H₁₅ClFN₇ ¹H NMR (400 MHz, DMSO-d₆): δ 4.59 (d, J = 5.64 Hz, 2H), 7.22 (t, J = 9.00 Hz, 1H), 7.32-7.35 (m, 1H), 7.52 (dd, J = 6.6, 3.96 Hz, 1H), 7.67 (d, J = 5.2 Hz, 1H), 7.75 (d, J = 7.52 Hz, 1H), 7.91 (s, 1H), 8.01 (d, J = 6.56 Hz, 1H), 8.42 (d, J = 3.36 Hz, 1H), 8.58 (s, 1H), 8.86 (s, 2H), 9.18 (s, 1H), 9.49 (s, 1H). | 5-Bromo-N²- (3-chloro-4- fluoro- phenyl)-N⁴- (pyridin-3- ylmethyl)- pyrimidine- 2,4-diamine hydrochloride salt (Intermediate 40) |
| 40 | 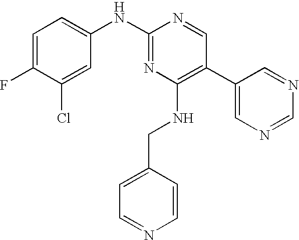 N²-(3-chloro-4- fluorophenyl)-N⁴- (pyridin-4-ylmethyl)- 5-pyrimidin-5- ylpyrimidine-2,4- diamine | MS(ES): 408 (M + 1) for C₂₀H₁₅ClFN₇ ¹H NMR (400 MHz, DMSO-d₆): δ 4.78 (d, J = 5.64 Hz, 2H), 7.28- 7.32 (m, 2H), 7.60 (br s, 1H), 7.97 (d, J = 6.44 Hz, 2H), 8.08 (s, 1H), 8.6 (br s, 1H), 8.83 (d, J = 6.64 Hz, 2H), 8.97 (s, 2H), 9.28 (s, 1H), 10.4 (br s, 1H). | 5-Bromo-N²- (3-chloro-4- fluoro- phenyl)-N⁴- pyridin-4- ylmethyl- pyrimidine- 2,4-diamine hydrochloride salt (Intermediate 41) |
| 41 | 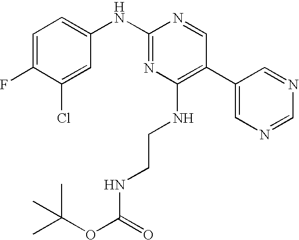 tert-butyl N-[2-[[2- [(3-chloro-4- fluorophenyl)amino]- 5-pyrimidin-5- ylpyrimidin-4- yl]amino]ethyl] carbamate | MS(ES): 460 (M + 1) for C₂₁H₂₃ClFN₇O₂ ¹H NMR (400 MHz, MeOD): δ 1.38 (s, 9H), 3.53 (t, J = 6.04 Hz, 2H), 7.16 (t, J = 9 Hz, 1H), 7.5 (m, 1H), 7.8 (s, 1H), 8.05 (dd, J = 6.74, 2.48 Hz, 1H), 8.85 (s, 2H), 9.14 (s, 1H). | {2-[5-Bromo- 2-(3-chloro- 4-fluoro- phenylamino)- pyrimidin-4- ylamino]- ethyl}- carbamic acid tert-butyl ester (Intermediate 73) |
| 42 | 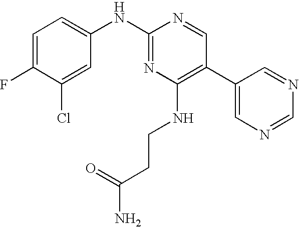 3-[[2-[(3-chloro-4- fluorophenyl)amino]- 5-pyrimidin-5- ylpyrimidin-4- yl]amino]propanamide | MS(ES): 388 (M + 1) for C₁₇H₁₅ClFN₇O. ¹H NMR (400 MHz, DMSO-d₆): δ 2.41 (t, J = 6.88 Hz, 2H), 3.56 (br s, 2H), 6.87 (br s, 1H), 7.17 (br s, 1H), 7.3 (t, J = 9.12 Hz, 1H), 7.34 (s, 1H), 7.7 (ddd, J = 9.06, 4.22, 2.72 Hz, 1H), 7.87 (s, 1H), 8.15 (dd, J = 6.86, 2.6 Hz, 1H), 8.8 (s, 2H), 9.16 (s, 1H), 9.54 (s, 1H). | 3-[5-Bromo- 2-(3-chloro- 4-fluoro- phenylamino)- pyrimidin-4- ylamino]- propionamide (Intermediate 42) |
| 43 | 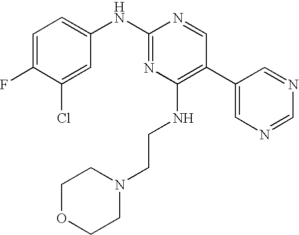 N²-(3-chloro-4- fluorophenyl)-N⁴-(2- morpholin-4-ylethyl)- 5-pyrimidin-5- ylpyrimidine-2,4- diamine | MS(ES): 430 (M + 1) for C₂₀H₂₁ClFN₇O. ¹H NMR (400 MHz, DMSO-d₆): δ 2.3-2.4 (m, 4H), 2.51-2.53 (m, 2H), 3.48-3.49 (m, 2H), 3.53-3.55 (m, 4H), 6.92 (t, J = 5.88 Hz, 1H), 7.29 (t, J = 9.04 Hz, 1H), 7.61- 7.65 (m, 2H), 7.88 (s, 1H), 8.18 (dd, J = 6.84, 2.44 Hz, 1H), 8.83 (s, 2H), 9.17 (s, 1H), 9.48 (s, 1H). | 5-Bromo-N²- (3-chloro-4- fluoro- phenyl)-N⁴- (2- morpholin-4- yl-ethyl)- pyrimidine- 2,4-diamine (Intermediate 43) |
| 44 | 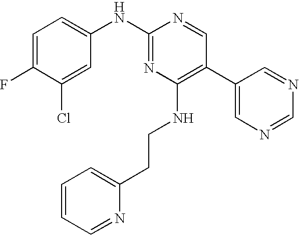 N²-(3-chloro-4- fluorophenyl)-N⁴-(2- pyridin-2-ylethyl)-5- pyrimidin-5- ylpyrimidine-2,4- diamine | MS(ES): 422.2 (M) for C₂₁H₁₇ClFN₇. ¹H NMR (400 MHz, DMSO-d₆): δ 3.04 (t, J = 7.12 Hz, 2H), 3.72 (q, J = 6.72 Hz, 2H), 7.20-7.27 (m, 4H), 7.66-7.71 (m, 2H), 7.86 (s, 1H), 8.17 (dd, J = 6.97, 2.68 Hz, 1H), 8.46 (dd, J = 4.82, 0.84 Hz, 1H), 8.75 (s, 2H), 9.15 (s, 1H), 9.49 (s, 1H). | 5-Bromo-N²- (3-chloro-4- fluoro- phenyl)-N⁴- (2-pyridin-2- yl-ethyl)- pyrimidine- 2,4-diamine (Intermediate 44) |
| 45 | 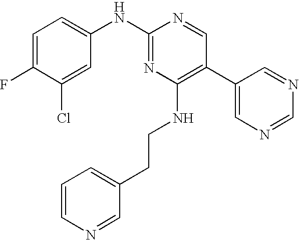 N²-(3-chloro-4- fluorophenyl)-N⁴-(2- pyridin-3-ylethyl)-5- pyrimidin-5- ylpyrimidine-2,4- diamine | MS(ES): 422.2 (M) for C₂₁H₁₇ClFN₇. ¹H NMR (400 MHz, DMSO-d₆): δ 2.91 (t, J = 7.12 Hz, 2H), 3.58- 3.63 (m, 2H), 7.18 (t, J = 5.12 Hz, 1H), 7.27 (d, J = 9.08 Hz, 1H), 7.30-7.35 (m, 1H), 7.59-7.66 (m, 2H), 7.87 (s, 1H), 8.20 (dd, J = 6.86, 2.48 Hz, 1H), 8.42-8.45 (m, 2H), 8.73 (s, 2H), 9.15 (s, 1H), 9.52 (s, 1H). | 5-Bromo-N²- (2-chloro-4- fluoro- phenyl)-N⁴- (2-pyridin-3- yl-ethyl)- pyrimidine- 2,4-diamine (Intermediate 45) |
| 46 | 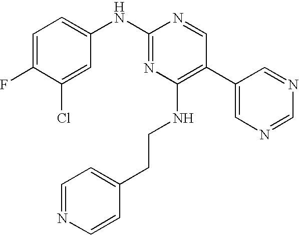 N²-(3-chloro-4- fluorophenyl)-N⁴-(2- pyridin-4-ylethyl)-5- pyrimidin-5- ylpyrimidine-2,4- diamine | MS(ES): 422.2 (M) for C₂₁H₁₇ClFN₇. ¹H NMR (400 MHz, DMSO-d₆): δ 2.92 (t, J = 7.16 Hz, 2H), 3.59- 3.64 (m, 2H), 7.14-7.17 (m, 1H), 7.26-7.30 (m, 3H), 7.58-7.62 (m, 1H), 7.87 (s, 1H), 8.18 (dd, J = 6.88, 2.68 Hz, 1H), 8.47 (dd, J = 4.52, 1.48 Hz, 2H), 8.73 (s, 2H), 9.15 (s, 1H), 9.51 (s, 1H). | 5-Bromo-N²- (3-chloro-4- fluoro- phenyl)-N⁴- (2-pyridin-4- yl-ethyl)- pyrimidine- 2,4-diamine hydrochloride salt (Intermediate 46) |
| 47 | 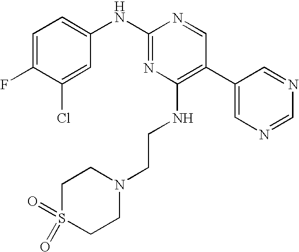 N²-(3-chloro-4- fluorophenyl)-N⁴-[2- (1,1-dioxo-1,4- thiazinan-4-yl)ethyl]- 5-pyrimidin-5- ylpyrimidine-2,4- diamine | MS(ES): 478 (M + 1) for C₂₀H₂₁ClFN₇O₂S. ¹H NMR (400 MHz, DMSO-d₆): δ 2.71 (br s, 2H), 2.95 (br s, 4H), 3.06 (br s, 4H), 3.47 (br s, 2H), 7.02 (br s, 1H), 7.32 (t, J = 9.04 Hz, 1H), 7.62 (ddd, J = 9.06, 4.22, 2.72 Hz, 1H), 7.89 (s, 1H), 8.15 (dd, J = 6.84, 2.6 Hz, 1H), 8.83 (s, 2H), 9.18 (s, 1H), 9.53 (s, 1H). | 5-Bromo-N²- (3-chloro-4- fluoro- phenyl)-N⁴- [2-(1,1- dioxo-1λ⁶- thiomorpholin- 4-yl)- ethyl]- pyrimidine- 2,4-diamine (Intermediate 47) |
| 48 | 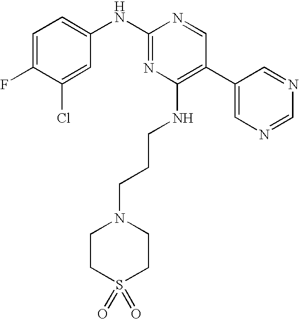 N²-(3-chloro-4- fluorophenyl)-N⁴-[3- (1,1-dioxo-1,4- thiazinan-4- yl)propyl]-5- pyrimidin-5- ylpyrimidine-2,4- diamine | MS(ES): 492 (M + 1) for. C₂₁H₂₃ClFN₇O₂S. ¹H 400 MHz, DMSO-d₆): δ 1.69- 1.73 (m, 2H), 2.50-2.52 (m, 2H), 2.82 (br s, 4H), 3.0 (br s, 4H), 3.36-3.38 (br s, 2H), 7.0 (br s, 1H), 7.30 (t, J = 9.12 Hz, 1H), 7.55-7.6 (m, 1H), 7.83 (s, 1H), 8.24 (dd, J = 2.44, 6.88 Hz, 1H), 8.79 (s, 2H), 9.1 (s, 1H), 9.47 (s, 1H). | 5-Bromo-N²- (3-chloro-4- fluoro- phenyl)-N⁴- [3-(1,1- dioxo-1λ⁶- thiomorpholin- 4-yl)- propyl]- pyrimidine- 2,4-diamine (Intermediate 49) |
| 49 | 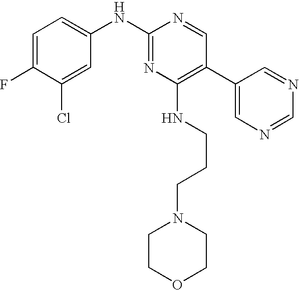 N²-(3-chloro-4- fluorophenyl)-N⁴-(3- morpholin-4- ylpropyl)-5- pyrimidin-5- ylpyrimidine-2,4- diamine | MS(ES): 444 (M + 1) for C₂₁H₂₃ClFN₇O. ¹H NMR (400 MHz, DMSO-d₆): δ 1.73 (br s, 2H), 2.32 (br s, 5H), 3.40 (q, J = 5.88 Hz, 2H), 3.48 (s, 4H), 7.09 (br s, 1H), 7.31 (t, J = 9.12 Hz, 1H), 7.59 (ddd, J = 2.72, 4.12, 9.07 Hz, 1H), 7.85 (s, 1H), 8.26 (dd, J = 2.40, 6.84 Hz, 1H), 8.81 (s, 2H), 9.17 (s, 1H), 9.50 (s, 1H). | 5-Bromo-N²- (3-chloro-4- fluoro- phenyl)-N⁴- (3- morpholin-4- yl-propyl)- pyrimidine- 2,4-diamine (Intermediate 48) |
| 50 | 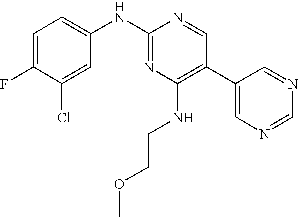 N²-(3-chloro-4- fluorophenyl)-N⁴-(2- methoxyethyl)-5- pyrimidin-5- ylpyrimidine-2,4- diamine | MS(ES): 375 (M + 1) for C₁₇H₁₆ClFN₆O ¹H NMR (400 MHz DMSO-d₆): δ 3.25 (s, 3H), 3.48-3.54 (m, 4H), 7.13 (br s, 1H), 7.27-7.32 (t, 1H, J = 9.1 Hz), 7.59-7.62 (m, 1H), 7.86 (s, 1H), 8.18-8.20 (dd, J = 3.96, 6.92 Hz, 1H), 8.78 (s, 2H), 9.16 (s, 1H), 9.52 (br s, 1H). | 5-Bromo-N²- (3-chloro-4- fluoro- phenyl)-N⁴- (2-methoxy- ethyl)- pyrimidine- 2,4-diamine (Intermediate 50) |
| 51 | 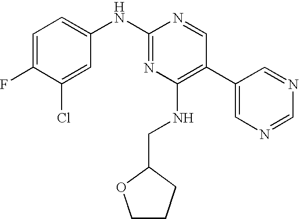 N-(3-chloro-4- fluorophenyl)-N′- (oxolan-2-ylmethyl)- 5-pyrimidin-5- ylpyrimidine-2,4- diamine | MS(ES): 401 (M + 1) for C₁₉H₁₈ClFN₆O. ¹H NMR (400 MHz DMSO-d₆): δ 1.57-1.60 (t, J = 11.92 Hz, 1H), 1.77-1.91 (m, 3H), 3.38-3.42 (m, 2H), 3.60-3.63 (m, 1H), 3.72- 3.75 (m, 1H), 4.07-4.10 (m, 1H), 7.0-7.12 (t, J = 11.04 Hz, 1H), 7.27-7.31 (t, J = 18.2 Hz, 1H), 7.58-7.62 (m, 1H), 7.85 (s, 1H), 8.19-8.21 (q, J = 9.48 Hz, 1H), 8.78 (s, 2H), 9.16 (s, 1H), 9.49 (s, 1H). | 5-Bromo-N²- (3-chloro-4- fluoro- phenyl)-N⁴- (tetrahydro- furan-2- ylmethyl)- pyrimidine- 2,4-diamine (Intermediate 75) |
| 52 | 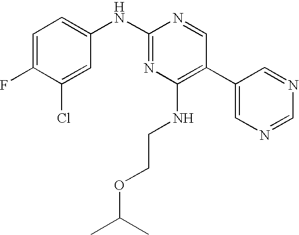 N²-(3-chloro-4- fluorophenyl)-N4-(2- propan-2- yloxyethyl)-5- pyrimidin-5- ylpyrimidine-2,4- diamine | MS(ES): 403 (M + 1) for C₁₉H₂₀ClFN₆O. ¹H NMR (400 MHz, DMSO-d₆): δ 1.06 (d, J = 6.08 Hz, 6H), 3.48- 3.57 (m, 5H), 7.06 (m, 1H), 7.29 (t, J = 9.12 Hz, 1H), 7.62 (ddd, J = 9.06, 4.24, 2.72 Hz, 1H), 7.87 (s, 1H), 8.19 (dd, J = 6.92, 2.64 Hz, 1H), 8.80 (s, 2H), 9.17 (s, 1H), 9.52 (s, 1H). | 5-Bromo-N²- (3-chloro-4- fluoro- phenyl)-N⁴- (2- isopropoxy- ethyl)- pyrimidine- 2,4-diamine (Intermediate 51) |
| 53 | 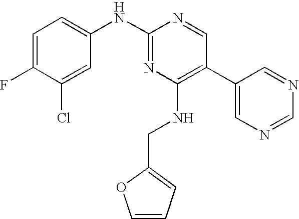 N²-(3-chloro-4- fluorophenyl)-N⁴- (furan-2-ylmethyl)-5- pyrimidin-5- ylpyrimidine-2,4- diamine | MS(ES): 397 (M + 1) for C₁₉H₁₄ClFN₆O ¹H NMR (400 MHz DMSO-d₆): δ 4.55 (d, J = 5.68 Hz, 2H), 6.28 (d, J = 2.88 Hz, 1H), 6.37 (dd, J = 3.10, 1.84 Hz, 1H), 7.29 (t, J = 9.08 Hz, 1H), 7.56-7.66 (m, 3H), 7.90 (s, 1H), 8.11 (dd, J = 6.86, 2.52 Hz, 1H), 8.81 (s, 2H), 9.18 (s, 1H), 9.57 (s, 1H). | 5-Bromo-N²- (3-chloro-4- fluoro- phenyl)-N⁴- (furan-2- ylmethyl)- pyrimidine- 2,4-diamine (Intermediate 52) |
| 54 | 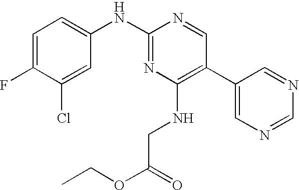 ethyl 2-[[2-[(3- chloro-4- fluorophenyl)amino]- 5-pyrimidin-5- ylpyrimidin-4- yl]amino]acetate | MS(ES): 403 (M + 1) for C₁₈H₁₆ClFN₆O₂. ¹H NMR (400 MHz MeOD): δ 1.22 (t, J = 7.08 Hz, 3H), 4.15 (m, 4H), 7.15 (t, J = 8.96 Hz, 1H), 7.45-7.48 (m, 1H), 7.89-7.93 (m, 2H), 8.90 (s, 2H), 9.17 (s, 1H). | [5-Bromo-2- (3-chloro-4- fluoro- phenylamino)- pyrimidin-4- ylamino]- acetic acid ethyl ester (Intermediate 76) |
| 55 | 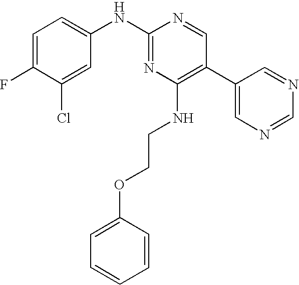 N²-(3-chloro-4- fluorophenyl)-N⁴-(2- phenoxyethyl)-5- pyrimidin-5- ylpyrimidine-2,4- diamine | MS(ES): 437 (M + 1) for C₂₂H₁₈ClFN₆O. ¹H NMR (400 MHz DMSO-d₆): δ 3.75 (m, 2H), 4.14 (t, J = 6.2 Hz, 2H), 6.9 (m, 3H), 7.26 (m, 5H), 7.61 (m, 1H), 7.89 (s, 1H), 8.2 (dd, J = 2.6 Hz, 1H), 8.72 (s, 2H), 9.17 (s, 1H), 9.53 (s, 1H). | 5-Bromo-N²- (3-chloro-4- fluoro- phenyl)-N⁴- (2-phenoxy- ethyl)- pyrimidine- 2,4-diamine (Intermediate 53) |
| 56 | 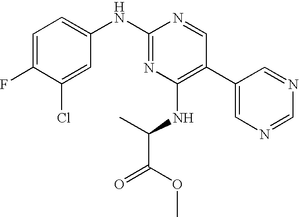 methyl (2R)-2-[[2- [(3-chloro-4- fluorophenyl)amino]- 5-pyrimidin-5- ylpyrimidin-4- yl]amino]propanoate | MS(ES): 403 (M + 1) for C₁₈H₁₆ClFN₆O₂. ¹H NMR (400 MHz, DMSO-d₆): δ 1.40 (d, J = 7.32 Hz, 3H), 3.58 (s, 3H), 4.73 (t, J = 7.28 Hz, 1H), 7.28 (t, J = 9.04 Hz, 1H), 7.34 (d, J = 7.40 Hz, 1H), 7.59 (m, 1H), 7.93 (s, 1H), 8.02 (dd, J = 6.76, 4.44 Hz, 1H), 8.83 (s, 1H), 9.17 (s, 1H), 9.51 (s, 1H). | Methyl (2R)- 2-[[2-[(3- chloro-4- fluorophenyl) amino]-5- bromo- pyrimidin-4- yl]amino] propanoate (Intermediate 54) |
| 57 | 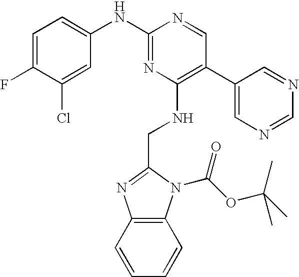 tert-butyl 2-[[[2-[(3- chloro-4- fluorophenyl)amino]- 5-pyrimidin-5- ylpyrimidin-4- yl]amino]methyl] benzimidazole-1- carboxylate | MS(ES): 547 for C₂₇H₂₄ClFN₈O₂ ¹H NMR (400 MHz, DMSO-d₆): δ 1.66 (s, 9H), 5.00 (d, J = 5.56 Hz, 2H), 7.09 (t, J = 9.08 Hz, 1H), 7.28-7.38 (m, 3H), 7.65 (d, J = 7.84 Hz, 2H), 7.86 (br s, 1H), 7.93 (d, J = 7.88 Hz, 1H), 7.99 (s, 1H), 8.31 (s, 1H), 8.98 (s, 2H), 9.20 (s, 1H), 9.50 (s, 1H). | N⁴-(1H- Benzoimidazol- 2- ylmethyl)-5- bromo-N²-(3- chloro-4- fluoro- phenyl)- pyrimidine- 2,4-diamine (Intermediate 126) |
| 58 | 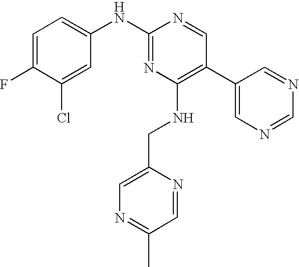 N²-(3-chloro-4- fluorophenyl)-N⁴-[(5- methylpyrazin-2- yl)methyl]-5- pyrimidin-5- ylpyrimidine-2,4- diamine | MS(ES): 423 (M + 1) for C₂₀H₁₆ClFN₈. ¹H NMR (400 MHz, DMSO-d₆) δ 2.44 (s, 3H), 4.66 (d, J = 5.72 Hz, 2H), 7.21 (t, J = 9.12 Hz, 1H), 7.50 (dt, J = 8.57, 4.00 Hz, 1H), 7.73 (s, 1H), 7.94 (m, 2H), 8.49 (d, J = 10.72 Hz, 2H), 8.89 (s, 2H), 9.19 (s, 1H), 9.49 (s, 1H). | 5-Bromo-N²- (3-chloro-4- fluoro- phenyl)-N⁴- (5-methyl- pyrazin-2- ylmethyl)- pyrimidine- 2,4-diamine (Intermediate 77) |
| 59 | 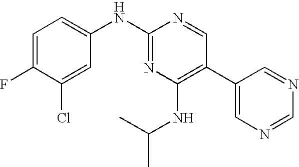 N²-(3-chloro-4- fluorophenyl)-N⁴- propan-2-yl-5- pyrimidin-5- ylpyrimidine-2,4- diamine | MS(ES): 359.1 (M + 1) for C₁₇H₂₆ClFN₆ ¹H NMR (400 MHz DMSO-d₆): δ 1.18 (d, J = 6.52 Hz, 6H), 4.30- 4.36 (m, 1H), 6.77 (d, J = 7.72 Hz, 1H), 7.3 (t, J = 9.08 Hz, 1H), 7.54-7.58 (m, 1H), 7.82 (s, 1H), 8.28 (dd, J = 6.92, 2.6 Hz, 1H), 8.78 (s, 2H), 9.15 (s, 1H), 9.48 (s, 1H). | (5-Bromo-2- chloro- pyrimidin-4- yl)-isopropyl- amine (Intermediate 56) |
| 60 | 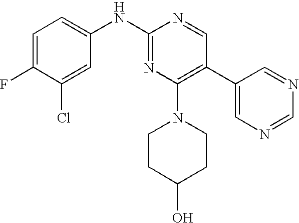 1-[2-[(3-chloro-4- fluorophenyl)amino]- 5-pyrimidin-5- ylpyrimidin-4- yl]piperidin-4-ol | MS(ES): 401 (M + 1) for C₁₉H₁₈ClFN₆O. ¹H NMR (400 MHz DMSO-d₆): δ 1.33 (m, 2H), 1.67 (m, 2H), 3.0 (m, 2H), 3.3 (m, 2H), 3.52 (m, 1H), 4.7 (br s, 1H), 7.33 (t, J = 9.12 Hz, 1H), 7.58 (ddd, 1H), 8.11 (s, 1H), 8.2 (dd, J = 2.64, 6.92 Hz, 1H), 8.9 (s, 2H), 9.11 (s, 1H), 9.7 (s, 1H). | 1-[5-Bromo- 2-(3-chloro- 4-fluoro- phenylamino)- pyrimidin-4- yl]-piperidin- 4-ol (Intermediate 78) |
| 61 | 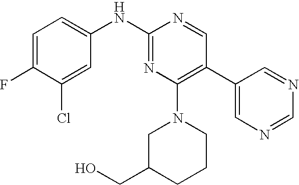 [1-[2-[(3-chloro-4- fluorophenyl)amino]- 5-pyrimidin-5- ylpyrimidin-4- yl]piperidin-3- yl]methanol | MS(ES): 413 (M − 1) for C₂₀H₂₀ClFN₆O. ¹H NMR (400 MHz DMSO-d₆): δ 1.13 (m, 1H), 1.42 (m, 1H), 1.56 (m, 2H), 1.67 (m, 1H), 2.58 (m, 1H), 2.82 (m, 1H), 3.10 (m, 1H), 3.20 (m, 1H), 3.65 (d, J = 3.52 Hz, 1H), 3.74 (d, J = 11.64 Hz, 1H), 4.41 (t, J = 4.96 Hz, 1H), 7.32 (t, J = 9.12 Hz, 1H), 7.66 (ddd, J = 9.08, 4.26, 2.72 Hz, 1H), 8.10 (s, 1H), 8.16 (dd, J = 6.86, 2.60 Hz, 1H), 8.88 (s, 2H), 9.10 (s, 1H), 9.68 (s, 1H). | {1-[5-Bromo- 2-(3-chloro- 4-fluoro- phenylamino)- pyrimidin-4- yl]-piperidin- 3-yl}- methanol (Intermediate 79) |
| 62 | 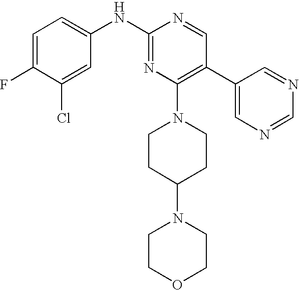 N-(3-chloro-4- fluorophenyl)-4-(4- morpholin-4- ylpiperidin-1-yl)-5- pyrimidin-5- ylpyrimidin-2-amine | MS(ES): 470.2 (M + 1) for C₂₃H₂₅ClFN₇O. ¹H NMR (400 MHz CDCl₃): δ 1.49 (m, 2H), 1.84 (m, 2H), 2.36 (m, 1H), 2.54 (br s, 4H), 2.84 (t, J = 12.12 Hz, 2H), 3.72 (br s, 4H), 3.86 (d, J = 13.72 Hz, 2H), 7.00 (d, J = 6.04 Hz, 1H), 7.10 (t, J = 8.76 Hz, 1H), 7.27 (m, 1H), 7.97 (s, 1H), 8.05 (dd, J = 6.60, 2.60 Hz, 1H), 8.83 (s, 2H), 9.17 (s, 1H). | [5-Bromo-4- (4- morpholin-4- yl-piperidin- 1-yl)- pyrimidin-2- yl]-(3-chloro- 4-fluoro- phenyl)- amine (Intermediate 80) |
| 63 | 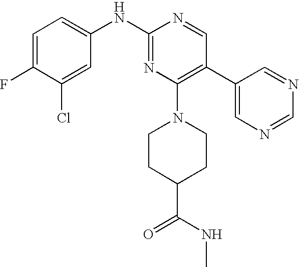 1-[2-[(3-chloro-4- fluorophenyl)amino]- 5-pyrimidin-5- ylpyrimidin-4-yl]-N- methylpiperidine-4- carboxamide | MS(ES): 442.3 (M + 1) for C₂₁H₂₁ClFN₇O ¹H NMR (400 MHz DMSO-d₆): δ 1.55 (m, 4H), 2.3 (m, 1H), 2.53 (d, J = 4.52 Hz, 3H), 2.82 (t, J = 10.6 Hz, 2H), 3.71 (d, J = 13.24 Hz, 2H), 7.3 (t, J = 9.08 Hz, 1H), 7.6 (ddd, J = 9.06, 4.2, 2.8 Hz, 1H), 7.72 (m, 1H), 8.13 (s, 1H), 8.21 (dd, J = 6.88, 2.6 Hz, 1H), 8.91 (s, 2H), 9.12 (s, 1H), 9.72 (s, 1H). | 1-[5-Bromo- 2-(3-chloro- 4-fluoro- phenylamino)- pyrimidin-4- yl]- piperidine-4- carboxylic acid methylamide (Intermediate 81) |
| 64 | 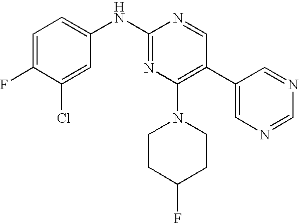 N-(3-chloro-4- fluorophenyl)-4-(4- fluoropiperidin-1-yl)- 5-pyrimidin-5- ylpyrimidin-2-amine | MS(ES): 403 (M + 1) for C₁₉H₁₇ClF₂N₆. ¹H NMR (400 MHz DMSO-d₆): δ 1.66 (m, 2H), 1.81-1.92 (m, 2H), 3.20 (m, 2H), 3.30 (m, 2H), 4.85 (d, J = 48.08 Hz, 1H), 7.34 (t, J = 9.08 Hz, 1H), 7.61 (m, 1H), 8.15- 8.19 (m, 2H), 8.93 (s, 2H), 9.13 (s, 1H), 9.73 (s, 1H). | [5-Bromo-4- (4-fluoro- piperidin-1- yl)- pyrimidin-2- yl]-(3-chloro- 4-fluoro- phenyl)- amine (Intermediate 82) |
| 65 | 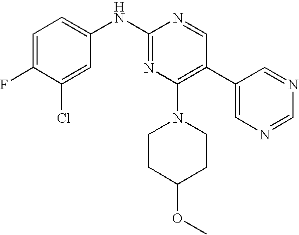 N-(3-chloro-4- fluorophenyl)-4-(4- methoxypiperidin-1- yl)-5-pyrimidin-5- ylpyrimidin-2-amine | MS(ES): 415.2 (M + 1) for C₂₀H₂₀ClFN₆O ¹H NMR (400 MHz DMSO-d₆): δ 1.35-1.43 (m, 2H), 1.78-1.81 (m, 2H), 2.99-3.05 (m, 2H), 3.21 (s, 3H), 3.47-3.5 (m, 2H), 7.33 (t, J = 9.12 Hz, 1H), 7.6 (ddd, J = 9.04, 4.22, 2.68 Hz, 1H), 8.12 (s, 1H), 8.2 (dd, J = 6.9, 2.6 Hz, 1H), 8.91 (s, 2H), 9.12 (s, 1H), 9.71 (s, 1H). | [5-Bromo-4- (4-methoxy- piperidin-1- yl)- pyrimidin-2- yl]-(3-chloro- 4-fluoro- phenyl)- amine (Intermediate 83) |
| 66 | 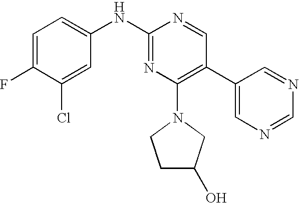 1-[2-[(3-chloro-4- fluorophenyl)amino]- 5-pyrimidin-5- ylpyrimidin-4- yl]pyrrolidin-3-ol | MS(ES): 387 (M + 1) for C₁₈H₁₆ClFN₆O. ¹H NMR (400 MHz DMSO-d₆): δ 1.65-1.77 (br s, 1H), 1.85-1.89 (m, 1H), 2.91 (br s, 1H), 3.25 (br s, 2H), 3.41 (br s, 1H), 4.22 (br s, 1H), 4.92 (br s, 1H), 7.32 (t, J = 9.12 Hz, 1H), 7.65 (ddd, J = 8.98, 4.14, 2.68 Hz, 1H), 7.97 (d, J = 1.44 Hz, 1H), 8.24 (dd, J = 6.92, 2.56 Hz, 1H), 8.79 (s, 2H), 9.13 (d, J = 1.32 Hz, 1H), 9.60 (s, 1H). | 1-[5-Bromo- 2-(3-chloro- 4-fluoro- phenylamino)- pyrimidin-4- yl]- pyrrolidin-3- ol (Intermediate 84) |
| 67 | 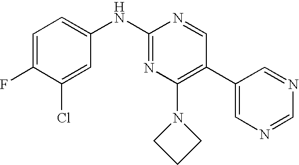 4-(azetidin-1-yl)-N- (3-chloro-4- fluorophenyl)-5- pyrimidin-5- ylpyrimidin-2-amine | MS(ES): 357 (M + 1) for C₁₇H₁₄ClFN₆. ¹H NMR (400 MHz DMSO-d₆): δ 2.19 (m, 2H), 3.79 (t, J = 7.36 Hz, 4H), 7.31 (t, J = 10.28 Hz, 1H), 7.64 (ddd, J = 9.06, 4.16, 2.76 Hz, 1H), 7.98 (s, 1H), 8.24 (dd, J = 6.92, 2.60 Hz, 1H), 8.80 (s, 2H), 9.13 (s, 1H), 9.64 (s, 1H). | (4-Azetidin- 1-yl-5- bromo- pyrimidin-2- yl)-(3-chloro- 4-fluoro- phenyl)- amine (Intermediate 87) |
| 68 | 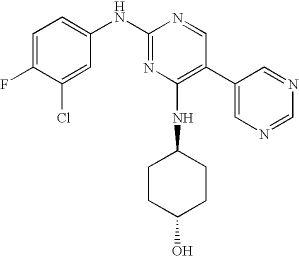 trans-4-[[2-[(3- chloro-4- fluorophenyl)amino]- 5-pyrimidin-5- ylpyrimidin-4- yl]amino]cyclohexan- 1-ol | MS(ES): 415 (M + 1) for C₂₀H₂₀ClFN₆O ¹H NMR (400 MHz, DMSO-d₆): δ 1.3 (m, 4H), 1.8 (m, 4H), 3.9 (m, 1H), 4.5 (d, J = 4.36 Hz, 1H), 6.7 (d, J = 7.6 Hz, 1H), 7.29 (t, J = 9.12 Hz, 1H), 7.54 (m, 1H), 7.82 (s, 1H), 8.26 (dd, J = 2.36, 6.84 Hz, 1H), 8.76 (s, 2H), 9.14 (s, 1H), 9.49 (s, 1H). | Trans-4-[5- Bromo-2-(3- chloro-4- fluoro- phenylamino)- pyrimidin-4- ylamino]- cyclohexanol (Intermediate 89) |
| 69 | 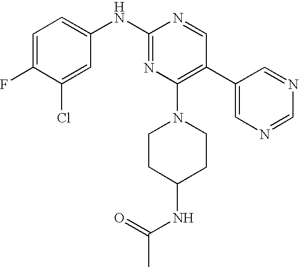 N-[1-[2-[(3-chloro-4- fluorophenyl)amino]- 5-pyrimidin-5- ylpyrimidin-4- yl]piperidin-4- yl]acetamide | MS(ES): 442.2 (M + 1) for C₂₁H₂₁ClFN₇O. ¹H NMR (400 MHz, DMSO-d₆): δ 1.30 (m, 2H), 1.66 (m, 2H), 1.75 (s, 3H), 2.93 (t, J = 11.60 Hz, 2H), 3.67 (d, J = 38.64 Hz, 2H), 3.73 (br s, 1H), 7.34 (t, J = 9.12 Hz, 1H), 7.61 (dt, J = 8.60, 4.16 Hz, 1H), 7.82 (d, J = 7.76 Hz, 1H), 8.14 (s, 1H), 8.19 (dd, J = 6.86, 2.56 Hz, 1H), 8.91 (s, 2H), 9.12 (s, 1H), 9.74 (s, 1H). | N-{1-[5- Bromo-2-(3- chloro-4- fluoro- phenylamino)- pyrimidin-4- yl]-piperidin- 4-yl}- acetamide (Intermediate 90) |
| 70 | 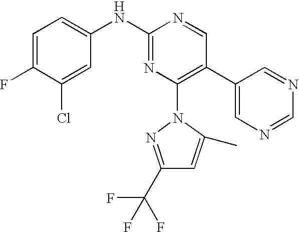 N-(3-chloro-4- fluorophenyl)-4-[5- methyl-3- (trifluoromethyl)-1H- pyrazol-1-yl]-5,5′- bipyrimidin-2-amine | MS(ES): 450 (M + 1) for C₁₉H₁₂ClF₄N₇. ¹H NMR (400 MHz, CDCl₃): δ 2.52 (s, 3H), 6.45 (s, 1H), 7.17 (t, J = 8.72 Hz, 1H), 7.36-7.40 (m, 1H), 7.44 (br s, 1H), 7.86 (dd, J = 2.60, 6.40 Hz, 1H), 8.47 (br s, 2H), 8.62 (br s, 1H), 9.17 (s, 1H). | 5-bromo-N- (3-chloro-4- fluorophenyl)- 4-[5-methyl- 3- (trifluoro- methyl)-1H- pyrazol-1- yl]pyrimidin- 2-amine (Intermediate 113) |
| 71 | 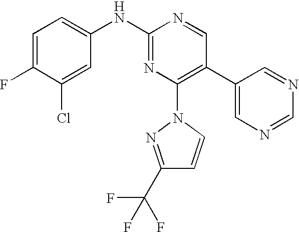 N-(3-chloro-4- fluorophenyl)-4-[3- (trifluoromethyl)-1H- pyrazol-1-yl]-5,5′- bipyrimidin-2-amine | MS(ES): 436 (M + 1) for C₁₈H₁₀ClF₄N₇. ¹H NMR (400 MHz DMSO-d₆): δ 7.07 (d, J = 2.68 Hz 1H), 7.43 (t, J = 9.12 Hz, 1H), 7.71-7.75 (m, 1H), 8.05 (dd, J = 6.64, 2.36 Hz, 1H), 8.60 (d, J = 1.76, 1H), 8.68 (s, 2H), 8.81 (s, 1H), 9.14 (s, 1H), 10.48 (s, 1H). | 5-bromo-N- (3-chloro-4- fluorophenyl)- 4-[3- (trifluoro- methyl)-1H- pyrazol-1- yl]pyrimidin- 2-amine (Intermediate 115) |
| 72 | 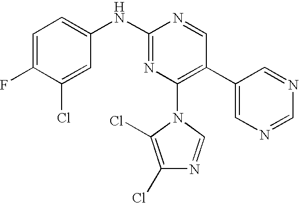 N-(3-chloro-4- fluorophenyl)-4-(4,5- dichloro-1H- imidazol-1-yl)-5,5′- bipyrimidin-2-amine | MS(ES): 436 (M + 1) for C₁₇H₉Cl₃FN₇. ¹H NMR (400 MHz DMSO-d₆): δ 7.43 (t, J = 9.08 Hz 1H), 7.68 (m, 1H), 8.05 (br s, 1H), 8.15 (s, 1H), 8.63 (s, 2H), 9.04 (s, 1H), 9.17 (s, 1H), 10.67 (s, 1H). | 5-bromo-N- (3-chloro-4- fluorophenyl)- 4-(4,5- dichloro-1H- imidazol-1- yl)pyrimidin- 2-amine (Intermediate 116) |
| 73 | 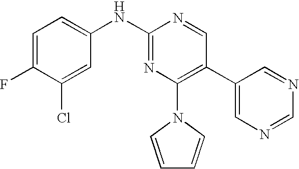 N-(3-chloro-4- fluorophenyl)-4-(1H- pyrrol-1-yl)-5,5′- bipyrimidin-2-amine | MS(ES): 367 (M + 1) for C₁₈H₁₂ClFN₆. ¹H NMR (400 MHz DMSO-d₆): δ 6.23 (t, J = 1.88 Hz, 2H), 6.94 (t, J = 1.88 Hz, 2H), 7.41 (t, J = 9.04 Hz, 1H), 7.68 (m, 1H), 8.11 (dd, J = 8.52, 2.44 Hz, 1H), 8.66 (s, 2H), 8.70 (s, 1H), 9.16 (s, 1H), 10.30 (s, 1H). | 5-bromo-N- (3-chloro-4- fluorophenyl)- 4-(1H- pyrrol-1- yl)pyrimidin- 2-amine (Intermediate 117) |
| 74 | 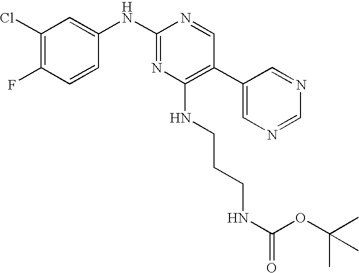 tert-butyl 3-(2-(3- chloro-4- fluorophenylamino)- 5,5′-bipyrimidin-4- ylamino)propylcar- bamate | MS(ES): 474 (M + 1) for C₂₂H₂₅ClFN₇O₂ ¹H NMR (300 MHz, DMSO-d6) δ ppm 1.33 (s, 9 H) 1.49-1.83 (m, 2 H) 2.79-3.12 (m, 2 H) 3.31- 3.49 (m, 2 H) 6.80 (t, J = 5.65 Hz, 1 H) 7.02 (t, J = 5.65 Hz, 1 H) 7.31 (t, J = 9.04 Hz, 1 H) 7.49-7.72 (m, 1 H) 7.84 (s, 1 H) 8.20 (dd, J = 6.69, 2.35 Hz, 1H) 8.80 (s, 2 H) 9.16 (s, 1 H) 9.49 (s, 1 H) | tert-Butyl 3- (5-bromo-2- (3-chloro-4- fluorophenyl amino) pyrimidin-4- ylamino) propyl- carbamate (Intermediate 97) |
| 75 | 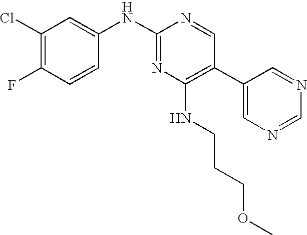 N²-(3-chloro-4- fluorophenyl)-N⁴-(3- methoxypropyl)-5,5′- bipyrimidin-2,4- diamine | MS(ES): 389 (M + 1) for C₁₈H₁₈ClFN₆O ¹H NMR (300 MHz, DMSO-d6) δ ppm 1.33 (t, J = 7.06 Hz, 3 H) 1.95 (s, 3 H) 3.24 (d, J = 10.93 Hz, 4 H) 3.42 (d, J = 11.49 Hz, 4 H) 4.37 (q, J = 6.97 Hz, 2 H) 7.34 (t, J = 9.14 Hz, 1 H) 7.49-7.79 (m, 1 H) 7.99-8.28 (m, 2 H) 8.28- 8.46 (m, 1 H) 8.92 (d, J = 2.07 Hz, 1 H) 9.01 (d, J = 1.88 Hz, 1 H) 9.75 (s, 1 H) | 5-bromo-N²- (3-chloro-4- fluorophenyl)- N⁴-(3- methoxy- propyl) pyrimidine- 2,4-diamine (Intermediate 119) |
| 76 | 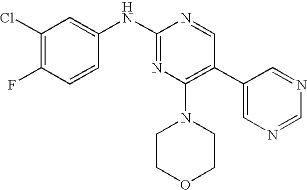 N-(3-chloro-4- fluorophenyl)-4- morpholin-5,5′- bipyrimidin-2-amine | MS(ES): 387 (M + 1) for C₁₈H₁₆ClFN₆O ¹H NMR (300 MHz, DMSO-d6) δ ppm 3.07-3.29 (m, 4 H) 3.41- 3.75 (m, 4 H) 7.33 (t, J = 9.14 Hz, 1 H) 7.51-7.75 (m, 1 H) 7.98- 8.29 (m, 2 H) 8.93 (s, 2 H) 9.12 (s, 1 H) 9.75 (s, 1 H) | 5-Bromo-N- (3-chloro-4- fluorophenyl)- 4- morpholin-4- ylpyrimidin- 2-amine (Intermediate 111) |

The following examples were prepared using the general method described above for Example 1 using (2-methoxypyrimidin-5-yl)boronic acid, tris(dibenzyledeneacetone)-dipalladium(0), 2-dicyclohexyl phosphino-2′,4′,6′-triiso-propyl-1,1′-biphenyl, sodium carbonate and the starting material (SM) indicated.

| Ex. | Compound | Data | SM |
|---|---|---|---|
| 77 | 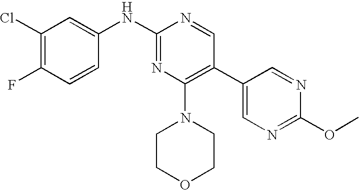 N-(3-chloro-4- fluorophenyl)-2′- methoxy-4- morpholin-5,5′- bipyrimidin-2-amine | MS(ES): 417 (M + 1) for C₁₉H₁₈ClFN₆O₂ 1H NMR (300 MHz, DMSO-d6) δ ppm 3.15-3.40 (m, 4 H) 3.46- 3.70 (m, 4 H) 3.94 (s, 3 H) 7.34 (t, J = 9.14 Hz, 1 H) 7.50-7.53 (m, 1 H) 7.97-8.20 (m, 2 H) 8.71 (s, 2 H) 9.77 (s, 1 H) | 5-Bromo-N- (3-chloro-4- fluorophenyl)- 4- morpholin-4- ylpyrimidin- 2-amine (Intermediate 111) |
| 78 | 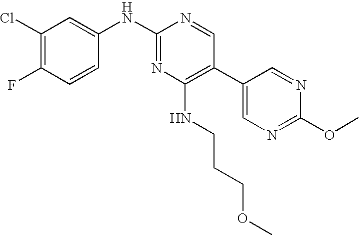 N²-(3-chloro-4- fluorophenyl)-2′- methoxy-N⁴-(3- methoxypropyl)-5,5′- bipyrimidine-2,4- diamine | MS(ES): 419 (M + 1) for C₁₉H₂₀ClFN₆O₂ 1H NMR (300 MHz, DMSO-d6) δ ppm 1.62-1.92 (m, 2 H) 3.08- 3.23 (m, 3 H) 3.26-3.54 (m, 4 H) 3.97 (s, 3 H) 7.41 (t, J = 9.04 Hz, 1 H) 7.47-7.61 (m, 1 H) 7.89 (s, 1 H) 7.96-8.21 (m, 2 H) 8.58 (s, 2 H) 10.59 (s, 1 H) | 5-bromo-N²- (3-chloro-4- fluorophenyl)- N⁴-(3- methoxy- propyl) pyrimidine- 2,4-diamine (Intermediate 119) |

### Example 79

### (Z)-5-(2-(3-chloro-4-fluorophenylamino)-4-(3-(dimethylamino)propylamino)pyrimidin-5-yl)-N′-hydroxynicotinimidamide

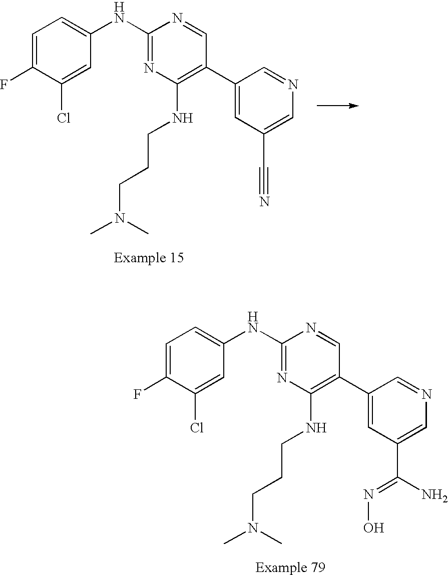

A stirred mixture of 5-{2-(3-chloro-4-fluorophenylamino)-4-[3-(dimethylamino)propylamino]pyrimidin-5-yl}nicotinonitrile (Example 15, 350 mg, 0.82 mmol) and aqueous hydroxylamine 50 weight % (0.097 mL, 1.64 mmol) in dioxane (3 mL) was prepared under nitrogen and heated to 80° C. After three hours, dioxane was removed under vacuum, and the residue was suspended in methanol. The mixture was cooled to near-zero while being stirred. The title compound was collected as a white solid (200 mg, 53%).

MS: ES+ 459 for C₂₁H₂₄ClFN₈O.

### Example 80

### 3-(5-(2-(3-chloro-4-fluorophenylamino)-4-(3-(dimethylamino)propylamino)pyrimidin-5-yl)pyridin-3-yl)-1,2,4-oxadiazol-5(4H)-one

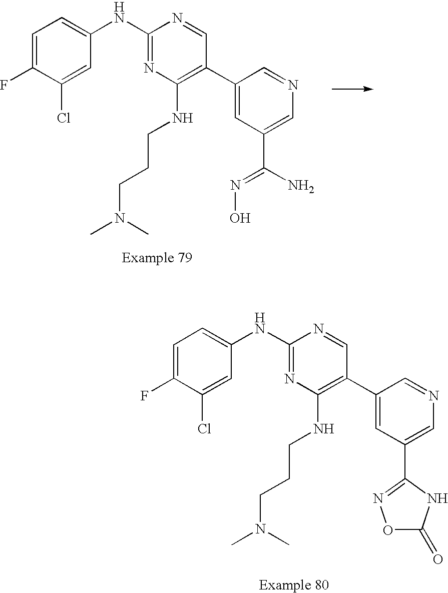

A stirred suspension of (Z)-5-(2-(3-chloro-4-fluorophenylamino)-4-(3-(dimethylamino)propylamino)pyrimidin-5-yl)-N′-hydroxynicotinimidamide (Example 79, 100 mg, 0.22 mmol), triethylamine (0.045 mL, 0.33 mmol), and 1,1′-carbonyldiimidazole (35 mg, 0.22 mmol) in dioxane (2 mL) were combined under nitrogen and heated to 80 degrees C. The suspension became a solution, and after 2 hours, the solvent was removed under vacuum. Reverse-phase chromatography (acetonitrile and water with ammonium acetate additive) was used to isolate the title compound (25 mg, 98%).

MS: ES+ 485 for C₂₂H₂₂ClFN₈O₂.

¹H NMR (300 MHz, DMSO-d₆) δ ppm 1.86-1.99 (m, 2H) 2.72 (s, 6 H) 2.98-3.39-3.51 (m, 2H) 7.06 (t, J=5.46 Hz, 1H) 7.33 (t, J=9.14 Hz, 1H) 7.56-7.70 (m, 1H) 7.90 (s, 1H) 8.13 (s, 1H) 8.21 (dd, J=6.88, 2.54 Hz, 1H) 8.60 (s, 1H) 8.92 (s, 1H) 9.50 (s, 1H).

### Example 81

### Sodium 3-(5-(2-(3-chloro-4-fluorophenylamino)-4-(3-(dimethylamino)propylamino)pyrimidin-5-yl)pyridin-3-yl)-5-oxo-1,2,4-oxadiazol-4-ide

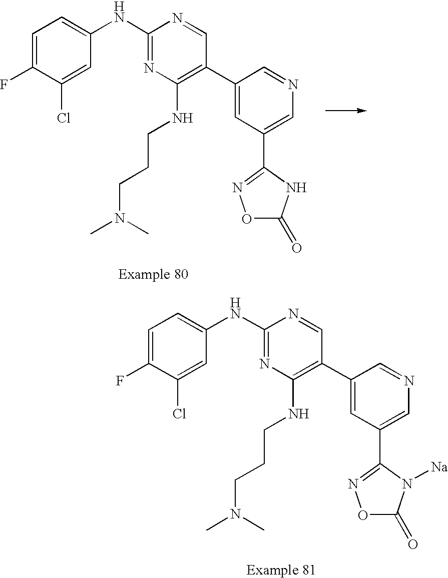

3-(5-(2-(3-Chloro-4-fluorophenylamino)-4-(3-(dimethylamino)propylamino)pyrimidin-5-yl)pyridin-3-yl)-1,2,4-oxadiazol-5(4H)-one (Example 80, 21 mg, 0.04 mmol) was dissolved in dioxane with stirring. 0.1 N NaOH (0.4 mL, 0.04 mmol) was added to the stirred solution. After another 20 minutes of stirring the solvent was removed under vacuum. The residue was placed under high vacuum overnight and characterized (21 mg, 91%).

MS: ES+485 for C₂₂H₂₂ClFN₈O₂ (free acid detected by LCMS).

¹H NMR (300 MHz, DMSO-d₆) δ ppm 0.70-0.86 (m, 2H) 1.12-1.26 (m, 2H) 1.59-1.74 (m, 2H) 2.06 (br. s., 6H) 7.17-7.34 (m, 2H) 7.53-7.64 (m, 1H) 7.77 (s, 1H) 7.98 (t, J=1.88 Hz, 1H) 8.18 (dd, 1H) 8.47 (d, J=2.07 Hz, 1H) 8.84 (d, J=1.70 Hz, 1 H) 9.39 (s, 1H).

### Example 82

### N-(3-chloro-4-fluorophenyl)-4-(3,5-dimethyl-1H-pyrazol-1-yl)-5,5′-bipyrimidin-2-amine

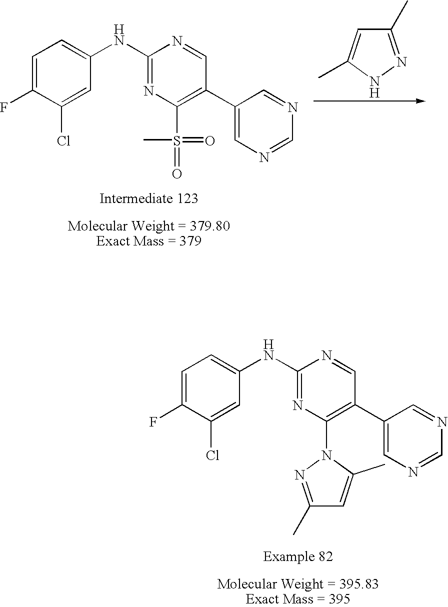

A solution of 3,5-dimethyl-1H-pyrazole (555 mg, 5.78 mmol, 2.2 eq) in DMF (2 mL) was added slowly to a suspension of sodium hydride (60%, 220 mg, 5.52 mmol, 2.1 eq) in DMF (1 mL) at 0° C. The reaction mixture was stirred for 25 min at room temperature. A solution of N-(3-chloro-4-fluorophenyl)-4-methylsulfonyl-5-pyrimidin-5-ylpyrimidin-2-amine (Intermediate 123, 1.0 g, 2.63 mmol, 1 eq) in DMF (1 mL) was added slowly to the reaction mixture, and the mixture was stirred for 1 h. Water was added to the reaction mixture (˜6 mL) and the solid formed was filtered and dried to provide the title compound (750 mg).

MS(ES): 396 (M+1) for C₁₉H₁₅ClFN₇.

¹H NMR 400 MHz, CDCl₃: δ 2.04 (s, 3H), 2.43 (s, 3H), 5.98 (s, 1H), 7.13 (t, J=8.72 Hz, 1H), 7.34-7.37 (m, 1H), 7.53 (br s, 1H), 7.86 (dd, J=2.52, 6.40 Hz, 1H), 8.45 (br s, 2H), 8.53 (s, 1H), 9.14 (br s, 1H).

The following examples were prepared using the general method described above for Example 82 using Intermediate 123, sodium hydride and the starting material (SM) indicated.

| Ex | Compound | Data | SM |
|---|---|---|---|
| 83 | 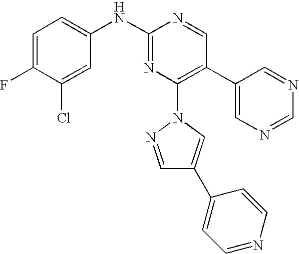 N-(3-chloro-4- fluorophenyl)-4- [4-(pyridin-4-yl)- 1H-pyrazol-1-yl]- 5,5′-bipyrimidin- 2-amine | MS(ES): 443 (M − 1) for C₂₂H₁₄ClFN₈. ¹H NMR (400 MHz, CH₃COOH): δ 7.32 (t, J = 8.80 Hz, 1H), 7.65 (m, 1H), 8.12-8.16 (m, 3H), 8.30 (s, 1H), 8.64 (s, 1H), 8.92-8.97 (m, 4H), 9.32 (s, 2H). | 4-(1H- Pyrazol-4- yl)-pyridine |
| 84 | 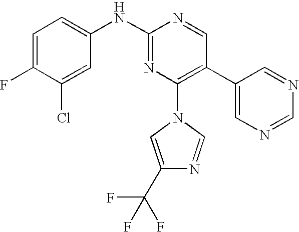 N-(3-chloro-4- fluorophenyl)-4- [4- (trifluoromethyl)- 1H-imidazol-1- yl]-5,5′- bipyrimidin-2- amine | MS(ES): 436 (M + 1) for C₁₈H₁₀ClF₄N₇. ¹H NMR (400 MHz, DMSO-d₆): δ 7.43 (t, J = 9.08 Hz, 1H), 7.70-7.72 (m, 1H), 7.95 (s, 1H), 8.08 (d, J = 4.8 Hz, 1H), 8.11 (s, 1H), 8.71 (s, 2H), 8.91 (s, 1H), 9.19 (s, 1H), 10.54 (br s, 1H). | 4- Trifluoromethyl- 1H-imidazole |
| 85 | 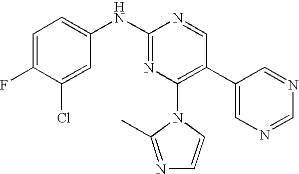 N-(3-chloro-4- fluorophenyl)-4- (2-methyl-1H- imidazol-1-yl)- 5,5′-bipyrimidin- 2-amine | MS(ES): 382 (M + 1) for C₁₈H₁₃ClFN₇. ¹H NMR (400 MHz, DMSO-d₆): δ 2.28 (s, 3H), 6.82 (s, 1H), 7.04 (s, 1H), 7.42 (t, J = 9.12 Hz, 1H), 7.65- 7.69 (m, 1H), 8.06 (d, J = 4.64 Hz, 1H), 8.55 (s, 2H), 8.93 (s, 1H), 9.12 (s, 1H), 10.46 (s, 1H). | 2-Methyl- 1H-imidazole |
| 86 | 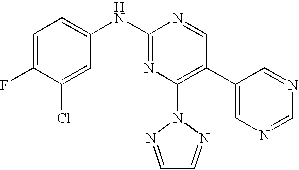 N-(3-chloro-4- fluorophenyl)-4- (2H-1,2,3-triazol- 2-yl)-5,5′- bipyrimidin-2- amine | MS(ES): 369 (M + 1) for C₁₆H₁₀ClFN₈. ¹H NMR (400 MHz, DMSO-d₆): δ 7.44 (t, J = 9.16 Hz, 1H), 7.79 (m, 1H), 8.14 (s, 2H), 8.25 (m, 1H), 8.65 (s, 2H), 8.85 (s, 1H), 9.13 (s, 1H), 10.59 (s, 1H). | 1H- [1,2,3]Triazole |
| 87 | 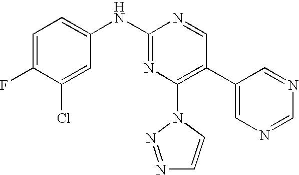 N-(3-chloro-4- fluorophenyl)-4- (2H-1,2,3-triazol- 1-yl)-5,5′- bipyrimidin-2- amine 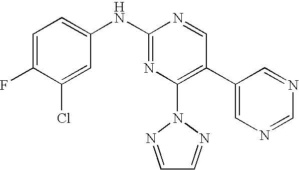 N-(3-chloro-4- fluorophenyl)-4- (2H-1,2,3-triazol- 2-yl)-5,5′- bipyrimidin-2- amine | MS(ES): 369 (M + 1) for C₁₆H₁₀ClFN₈. mixture of isomers ¹H NMR (400 MHz, DMSO-d₆): δ 7.39-7.45 (m, 1H), 7.71-7.75 (m, 1H), 7.97-8.14 (m, 2H), 8.65-8.68 (m, 3H), 8.90 (s, 1H), 9.14 (s, 1H), 10.59 (s, 1H). | 1H- [1,2,3]Triazole |
| 88 | 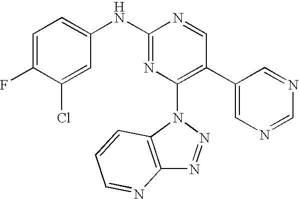 N-(3-chloro-4- fluorophenyl)-4- (1H- [1,2,3]triazolo[4,5- b]pyridin-1-yl)- 5,5′-bipyrimidin- 2-amine | MS(ES): 418 (M − 1) for C₁₉H₁₁ClFN₉. ¹H NMR (400 MHz, DMSO-d₆): δ 7.44 (t, J = 9.04 Hz, 1H), 7.61-7.65 (m, 1H), 7.83 (dd, J = 4.44, 8.40 Hz, 1H), 8.10-8.15 (m, 1H), 8.83-8.86 (m, 4H), 8.92 (s, 1H), 9.18 (s, 1H), 10.53 (br s, 1H). | 1H-[1,2,3]Triazolo [4,5-b]pyridine |
| 89 | 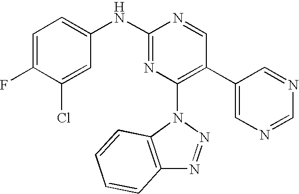 4-(1H- benzotriazol-1- yl)-N-(3-chloro-4- fluorophenyl)- 5,5′-bipyrimidin- 2-amine | MS(ES): 419 (M + 1) for C₂₀H₁₂ClFN₈. ¹H NMR (400 MHz, DMSO-d₆): δ 7.44 (t, J = 9.20 Hz, 1H), 7.59 (t, J = 8.00 Hz, 1H), 7.61-7.70 (m, 1H), 7.76 (t, J = 8.00 Hz, 1H), 8.16-8.19 (m, 2H), 8.40 (br s, 1H), 8.78 (s, 2H), 8.93 (s, 1H), 9.16 (s, 1H), 10.54 (s, 1H). | 1H- Benzotriazole |

The following examples were prepared using the general method described above for Example 1 using 3-(ethoxycarbonyl)pyridine-5-boronic acid pinacol ester, tris(dibenzyledeneacetone)-dipalladium(0), 2-dicyclohexyl phosphino-2′,4′,6′-triiso-propyl-1,1′-biphenyl, sodium carbonate and the starting material (SM) indicated.

| Ex | Compound | Data | SM |
|---|---|---|---|
| 90 | 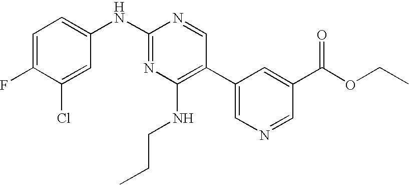 5-[2-(3-Chloro-4- fluoro-phenylamino)-4- propylamino- pyrimidin-5-yl]- nicotinic acid ethyl ester | MS(ES): 430.2 (M + 1) for C₂₁H₂₁ClFN₅O₂. ¹H NMR (400 MHz, DMSO- d₆): δ 0.9 (t, J = 7.44 Hz, 3H), 1.31-1.35 (m, 3H), 1.58 (q, J = 7.3 Hz, 2H), 4.35 (q, J = 7.12 Hz, 2H), 7.0 (t, J = 5.76 Hz, 1H), 7.29 (t, J = 9.08 Hz, 1H), 7.57 (ddd, J = 9.06, 4.2, 2.64 Hz, 1H), 7.83 (s, 1H), 8.21 (t, J = 2.12 Hz, 1H), 8.29 (dd, J = 6.92, 2.68 Hz, 1H), 8.77 (d, J = 2.2 Hz, 1H), 9.03 (d, J = 2 Hz, 1H), 9.48 (s, 1H). | 5-Bromo-N²- (3-chloro-4- fluoro- phenyl)-N⁴- propyl- pyrimidine- 2,4-diamine (Intermediate 35) |
| 91 | 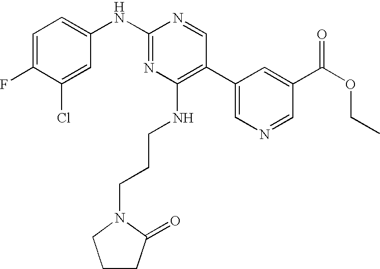 5-{2-(3-Chloro-4- fluoro-phenylamino)-4- [3-(2-oxo-pyrrolidin-1- yl)-propylamino]- pyrimidin-5-yl}- nicotinic acid ethyl ester | MS(ES): 513 (M + 1) for C₂₅H₂₆ClFN₆O₃. ¹H NMR (400 MHz, DMSO- d₆): δ 1.33 (t, J = 6.96 Hz, 3H), 1.74 (t, J = 6.40 Hz, 2H), 1.87 (t, J = 7.48 Hz, 2H), 2.16 (t, J = 7.88 Hz, 2H), 3.30 (m, 6H), 4.33 (m, 2H), 6.90 (br s, 1H), 7.33 (m, 1H), 7.65 (m, 1H), 7.85 (br s, 1H), 8.20 (m, 2H), 8.80 (br s, 1H), 9.05 (s, 1H), 9.50 (s, 1H). | 1-{3-[5- Bromo-2-(3- chloro-4- fluoro- phenylamino)- pyrimidin-4- ylamino]- propyl}- pyrrolidin-2- one (Intermediate 36) |
| 92 | 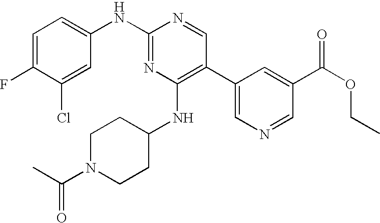 5-[4-(1-Acetyl- piperidin-4-ylamino)-2- (3-chloro-4-fluoro- phenylamino)- pyrimidin-5-yl]- nicotinic acid ethyl ester | MS(ES): 513 (M + 1) for C₂₅H₂₆ClFN₆O₃. ¹H NMR (400 MHz, DMSO- d₆): δ 1.33 (t, J = 7.08 Hz, 3H), 1.34-1.47 (m, 1H), 1.83-1.91 (m, 2H), 1.98 (s, 3H), 2.66 (m, 1H), 3.13 (m, 1H), 3.82 (d, J = 2.60 Hz, 1H), 4.22 (m, 1H), 4.33-4.38 (m, 3H), 6.69 (d, J = 7.76 Hz, 1H), 7.33 (t, J = 9.12 Hz, 1H), 7.56 (m, 1H), 7.87 (s, 1H), 8.21 (t, J = 2.08 Hz, 1H), 8.23 (dd, J = 7.00, 2.72 Hz, 1H), 8.78 (d, J = 2.12 Hz, 1H), 9.03 (d, J = 1.92 Hz, 1H), 9.53 (s, 1H). | 1-{4-[5- Bromo-2-(3- chloro-4- fluoro- phenylamino)- pyrimidin-4- ylamino]- piperidin-1- yl}-ethanone (Intermediate 72) |
| 93 | 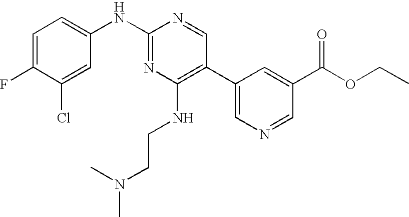 5-[2-(3-Chloro-4- fluoro-phenylamino)-4- (2-dimethylamino)- ethylamino)-pyrimidin- 5-yl]-nicotinic acid ethyl ester | MS(ES): 459.5 (M + 1) for C₂₂H₂₄ClFN₆O₂. ¹H NMR (400 MHz, MeOD): δ 1.43 (t, J = 7.12 Hz, 3H), 2.38 (s, 6H), 2.72 (t, J = 6.32 Hz, 2H), 3.64 (t, J = 6.48 Hz, 2H), 4.45 (q, J = 7.12 Hz, 2H), 7.16 (t, J = 9 Hz, 1H), 7.50 (ddd, J = 8.98, 4.1, 2.68 Hz, 1H), 7.82 (s, 1H), 8.01 (dd, J = 6.7, 2.64 Hz, 1H), 8.4 (t, J = 2.04 Hz, 1H), 8.78 (d, J = 2.08 Hz, 1H), 9.1 (d, J = 1.96 Hz, 1H). | 5-Bromo-N²- (3-chloro-4- fluoro- phenyl)-N⁴- (2- dimethylamino- ethyl)- pyrimidine- 2,4-diamine (Intermediate 37) |
| 94 | 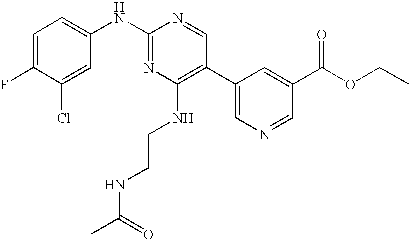 5-[4-(2-Acetylamino- ethylamino)-2-(3- chloro-4-fluoro- phenylamino)- pyrimidin-5-yl]- nicotinic acid ethyl ester | MS(ES): 473 (M + 1) for C₂₂H₂₂ClFN₆O₃. ¹H NMR (400 MHz, MeOD): δ 1.43 (t, J = 7.12 Hz, 3H), 1.91 (s, 3H), 3.44 (t, J = 5.52 Hz, 2H), 3.57 (t, J = 6.24 Hz, 2H), 4.46 (q, J = 7.12 Hz, 2H), 7.17 (t, J = 9 Hz, 1H), 7.51 (ddd, J = 8.98, 4.1, 2.68 Hz, 1H), 7.8 (s, 1H), 8.06 (dd, J = 6.76, 2.68 Hz, 1H), 8.40 (t, J = 2.08 Hz, 1H), 8.78 (d, J = 2.2 Hz, 1H), 9.11 (d, J = 1.96 Hz, 1H). | N-{2-[5- Bromo-2-(3- chloro-4- fluoro- phenylamino)- pyrimidin-4- ylamino]- ethyl}- acetamide (Intermediate 38) |
| 95 | 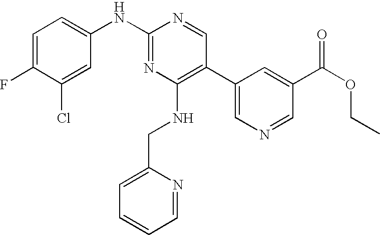 5-{2-(3-Chloro-4- fluoro-phenylamino)-4- [(pyridin-2-ylmethyl)- amino]-pyrimidin-5- yl}-nicotinic acid ethyl ester | MS(ES): 479. (M + 1) for C₂₄H₂₀ClFN₆O₂. ¹H NMR (400 MHz, DMSO- d₆): δ 1.34 (t, J = 7.12 Hz, 3H), 4.38 (q, J = 7.08 Hz, 2H), 4.68 (d, J = 5.8 Hz, 2H), 7.16 (t, J = 9.12 Hz, 1H), 7.24 (dd, J = 7.02, 5.48 Hz, 1H), 7.33 (d, J = 7.68 Hz, 1H), 7.49-7.55 (m, 2H), 7.71-7.75 (m, 1H), 7.93-7.95 (m, 2H), 8.35 (t, J = 2.05 Hz, 1H), 8.52 (d, J = 4.92 Hz, 1H), 8.9 (d, J = 2.8 Hz, 1H), 9.07 (d, J = 1.88 Hz, 1H), 9.43 (s, 1H). | 5-Bromo-N²- (3-chloro-4- fluoro- phenyl)-N⁴- pyridin-2- ylmethyl- pyrimidine- 2,4-diamine (Intermediate 39) |
| 96 | 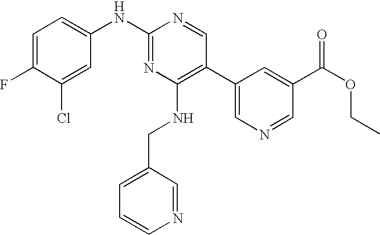 5-{2-(3-Chloro-4- fluoro-phenylamino)-4- [(pyridin-3-ylmethyl)- amino]-pyrimidin-5- yl}-nicotinic acid ethyl ester | MS(ES): 479.5 (M + 1) for C₂₄H₂₀ClFN₆O₂. ¹H NMR (400 MHz, MeOD): δ 1.42 (t, J = 7.12 Hz, 3H), 4.45 (q, J = 7.12 Hz, 2H), 4.70 (s, 2H), 7.10 (t, J = 8.96 Hz, 1H), 7.33-7.41 (m, 2H), 7.8-7.9 (m, 3H), 8.41-8.44 (m, 2H), 8.5 (s, 1H), 8.81 (d, J = 2.08 Hz, 1H), 9.13 (d, J = 1.92 Hz, 1H). | 5-Bromo-N²- (3-chloro-4- fluoro- phenyl)-N⁴- (pyridin-3- ylmethyl)- pyrimidine- 2,4-diamine hydrochloride salt (Intermediate 40) |
| 97 | 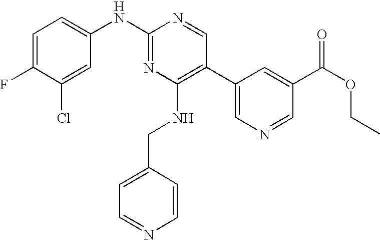 5-{2-(3-Chloro-4- fluoro-phenylamino)-4- [(pyridin-4-ylmethyl)- amino]-pyrimidin-5- yl}-nicotinic acid ethyl ester | MS(ES): 479.5 (M + 1) for C₂₄H₂₀ClFN₆O₂. ¹H NMR (400 MHz, DMSO- d₆): δ 1.34 (t, J = 7.08 Hz, 3H), 4.38 (q, J = 7.04 Hz, 2H), 4.59 (d, J = 5.92 Hz, 2H), 7.18 (t, J = 9.12 Hz, 1H), 7.34 (d, J = 5.96 Hz, 2H), 7.44-7.48 (m, 1H), 7.57 (t, J = 5.96 Hz, 1H), 7.92 (m, 2H), 8.31 (t, J = 2.12 Hz, 1H), 8.49 (dd, J = 4.48, 1.56 Hz, 2H), 8.89 (d, J = 2.20 Hz, 1H), 9.07 (d, J = 2.00 Hz, 1H), 9.45 (s, 1H). | PE-004-025 5-Bromo-N²- (3-chloro-4- fluoro- phenyl)-N⁴- pyridin-4- ylmethyl- pyrimidine- 2,4-diamine hydrochloride salt (Intermediate 41) |
| 98 | 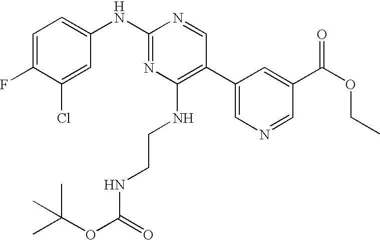 5-[4-(2-tert- Butoxycarbonylamino- ethylamino)-2-(3- chloro-4-fluoro- phenylamino)- pyrimidin-5-yl]- nicotinic acid ethyl ester | MS (ES): 531 (M + 1) for C₂₅H₂₈ClFN₆O₄. ¹H NMR (400 MHz, MeOD): δ 1.33 (s, 9H), 1.43 (t, J = 7.12 Hz, 3H), 3.54 (t, J = 6.08 Hz, 2H), 4.45 (q, J = 7.12 Hz, 2H), 7.17 (t, J = 8.96 Hz, 1H), 7.52 (dt, J = 8.84, 3.88 Hz, 1H), 7.80 (s, 1H), 8.05 (dd, J = 6.70, 2.64 Hz, 1H), 8.39 (t, J = 2.04 Hz, 1H), 8.77 (d, J = 2.12 Hz, 1H), 9.10 (d, J = 1.96 Hz, 1H). | {2-[5-Bromo- 2-(3-chloro- 4-fluoro- phenylamino)- pyrimidin-4- ylamino]- ethyl}- carbamic acid tert-butyl ester (Intermediate 73) |
| 99 | 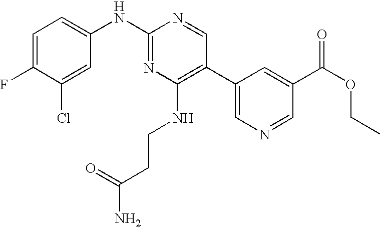 5-[4-(2-Carbamoyl- ethylamino)-2-(3- chloro-4-fluoro- phenylamino)- pyrimidin-5-yl]- nicotinic acid ethyl ester | MS(ES): 459 (M + 1) for C₂₁H₂₀ClFN₆O₃. ¹H NMR (400 MHz, MeOD): δ 1.43 (t, J = 7.16 Hz, 3H), 2.59 (t, J = 6.76 Hz, 2H), 3.73 (t, J = 6.72 Hz, 2H), 4.44 (q, J = 7.12 Hz, 2H), 7.16 (t, J = 9 Hz, 1H), 7.55 (ddd, J = 9, 4.06, 2.76 Hz, 1H), 7.81 (s, 1H), 8.04 (dd, J = 6.74, 2.68 Hz, 1H), 8.38 (t, J = 2.04 Hz, 1H), 8.75 (d, J = 2.16 Hz, 1H), 9.1 (d, J = 1.92 Hz, 1H). | 3-[5-Bromo- 2-(3-chloro- 4-fluoro- phenylamino)- pyrimidin-4- ylamino]- propionamide (Intermediate 42) |
| 100 | 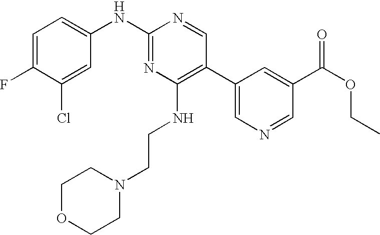 5-[2-(3-Chloro-4- fluoro-phenylamino)-4- (2-morpholin-4-yl- ethylamino)-pyrimidin- 5-yl]-nicotinic acid ethyl ester | MS(ES): 501 (M + 1) for C₂₄H₂₆ClFN₆O₃. ¹H NMR (400 MHz, DMSO- d₆): δ 1.34 (t, J = 7.08 Hz, 3H), 2.31 (s, 4H), 3.45-3.53 (m, 6H), 4.37 (q, J = 7.08 Hz, 2H), 6.70 (m, 1H), 7.29 (t, J = 9.16 Hz, 1H), 7.61-7.64 (m, 1H), 7.87 (s, 1H), 8.18 (dd, J = 6.92, 2.64 Hz, 1H), 8.24 (t, J = 2.08 Hz, 1H), 8.81 (d, J = 2.20 Hz, 1H), 9.05 (d, J = 2.00 Hz, 1H), 9.48 (s, 1H). | 5-Bromo-N²- (3-chloro-4- fluoro- phenyl)-N⁴- (2- morpholin-4- yl-ethyl)- pyrimidine- 2,4-diamine (Intermediate 43) |
| 101 | 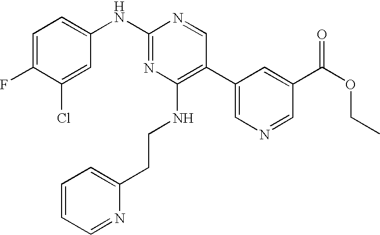 5-[2-(3-Chloro-4- fluoro-phenylamino)-4- (2-pyridin-2-yl- ethylamino)-pyrimidin- 5-yl]-nicotinic acid ethyl ester | MS (ES): 493 (M) for C₂₅H₂₂ClFN₆O₂. ¹H NMR (400 MHz, DMSO- d₆): δ 1.33 (t, J = 7.12 Hz, 3H), 3.04 (t, J = 7.04 Hz, 2H), 3.72 (q, J = 5.84 Hz, 2H), 4.37 (q, J = 7.08 Hz, 2H), 7.08 (t, 1H), 7.23-7.27 (m, 3H), 7.69 (m, 2H), 7.85 (s, 1H), 8.17 (m, 2H), 8.44 (m, 1H), 8.73 (d, J = 2.24 Hz, 1H), 9.04 (d, J = 2.04 Hz, 1H), 9.48 (s, 1H). | 5-Bromo-N²- (3-chloro-4- fluoro- phenyl)-N⁴- (2-pyridin-2- yl-ethyl)- pyrimidine- 2,4-diamine (Intermediate 44) |
| 102 | 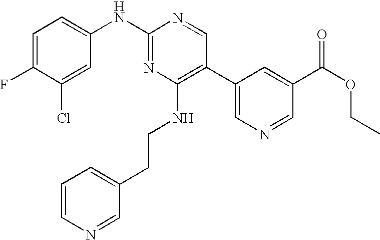 5-[2-(3-Chloro-4- fluoro-phenylamino)-4- (2-pyridin-3-yl- ethylamino)-pyrimidin- 5-yl]-nicotinic acid ethyl ester | MS(ES): 493 (M) for C₂₅H₂₂ClFN₆O₂. ¹H NMR (400 MHz, DMSO- d₆): δ 1.34 (t, J = 7.04 Hz, 3H), 2.89 (t, J = 7.16 Hz, 2H), 3.6 (q, J = 6.72 Hz, 2 H), 4.37 (q, J = 7.12 Hz, 2H), 7.01 (t, J = 5.52 Hz, 1H), 7.28 (m, 2H), 7.60 (m, 2H), 7.85 (s, 1H), 8.15 (t, J = 2.16 Hz, 1H), 8.20 (dd, J = 6.90, 2.56 Hz, 1H), 8.40 (m, 2H), 8.71 (d, J = 2.16 Hz, 1H), 9.03 (d, J = 1.96 Hz, 1H), 9.48 (s, 1H). | 5-Bromo-N²- (3-chloro-4- fluoro- phenyl)-N⁴- (2-pyridin-3- yl-ethyl)- pyrimidine- 2,4-diamine (Intermediate 45) |
| 103 | 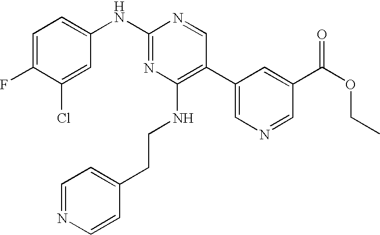 5-[2-(3-Chloro-4- fluoro-phenylamino)-4- (2-pyridin-4-yl- ethylamino)-pyrimidin- 5-yl]-nicotinic acid ethyl ester | MS(ES): 493 (M) for C₂₅H₂₂ClFN₆O₂. ¹H NMR (400 MHz, CD₃OD): δ 1.43 (t, J = 7.12 Hz, 3H), 3.00 (t, J = 7.32 Hz, 2H), 3.75 (t, J = 6.96 Hz, 2H), 4.45 (q, J = 7.08 Hz, 2H), 7.17 (t, J = 8.92 Hz, 1H), 7.30 (d, J = 6.0 Hz, 2H), 7.45-7.49 (m, 1H), 7.79 (s, 1H), 8.07 (dd, J = 2.68, 6.72 Hz, 1H), 8.32 (t, J = 2.00 Hz, 1H), 8.42 (d, J = 6.12 Hz, 2H), 8.69 (d, J = 2.08 Hz, 1H), 9.09 (d, J = 2.00 Hz, 1H). | 5-Bromo-N-²- (3-chloro-4- fluoro- phenyl)-N⁴- (2-pyridin-4- yl-ethyl)- pyrimidine- 2,4-diamine hydrochloride salt (Intermediate 46) |
| 104 | 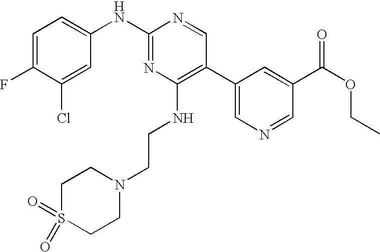 5-{2-(3-Chloro-4- fluoro-phenylamino)-4- [2-(1,1-dioxo-1λ⁶- thiomorpholin-4-yl)- ethylamino]-pyrimidin- 5-yl}-nicotinic acid ethyl ester | MS(ES): 549.2 (M + 1) for C₂₄H₂₆ClFN₆O₄S. ¹H NMR (400 MHz, DMSO- d₆): δ1.34 (t, J = 7.08 Hz, 3H), 2.70 (t, J = 6.40 Hz, 2H), 2.93 (br s, 4H), 3.03 (br s, 4H), 3.38- 3.48 (m, 2H), 3.47 (m, 2H), 4.38 (q, J = 7.04 Hz, 2H), 6.81 (t, J = 5.44 Hz, 1H), 7.31 (t, J = 9.12 Hz, 1H), 7.64 (ddd, J = 9.10, 4.24, 2.72 Hz, 1H), 7.88 (s, 1H), 8.18 (dd, J = 6.92, 2.60 Hz, 1H), 8.25 (t, J = 2.12 Hz, 1H), 8.82 (d, J = 2.20 Hz, 1H), 9.05 (d, J = 2.0 Hz, 1H), 9.48 (s, 1H). | 5-Bromo-N²- (3-chloro-4- fluoro- phenyl)-N⁴- [2-(1,1- dioxo-1λ⁶- thiomorpholin- 4-yl)- ethyl]- pyrimidine- 2,4-diamine (Intermediate 47) |
| 105 | 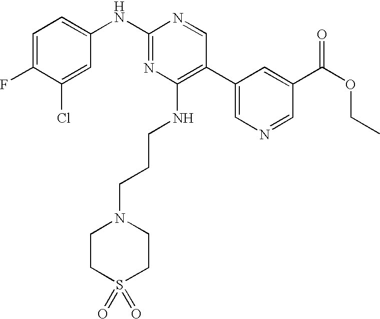 5-{2-(3-Chloro-4- fluoro-phenylamino)-4- [3-(1,1-dioxo-1λ⁶- thiomorpholin-4-yl)- propylamino]- pyrimidin-5-yl}- nicotinic acid ethyl ester | MS(ES): 563 (M + 1) for C₂₅H₂₈ClFN₆O₄S. ¹H NMR (400 MHz, DMSO- d₆): δ 1.34 (t, J = 7.04 Hz, 3H), 1.71-1.74 (t, 2H, J = 7 Hz), 2.50-2.54 (br s, 2H), 2.83 (br s, 4H), 2.99-3.0 (br s, 4H), 3.38- 3.42 (m, 2H), 4.37 (q, J = 7.04 Hz 2H), 6.95-6.97 (t, J = 5.44 Hz, 1H), 7.31 (t, J = 9.16 Hz, 1H), 7.60 (ddd, J = 9.02, 4.18, 2.76 Hz, 1H), 7.85 (s, 1H), 8.22 (t, J = 2.12 Hz, 1H), 8.27 (dd, J = 6.86, 2.60 Hz, 1H), 8.80 (d, J = 2.16 Hz, 1H), 9.0 (d, 1H, J = 1.96 Hz), 9.48 (s, 1H). | 5-Bromo-N²- (3-chloro-4- fluoro- phenyl)-N⁴- [3-(1,1- dioxo-1λ⁶- thiomorpholin- 4-yl)- propyl]- pyrimidine- 2,4-diamine (Intermediate 49) |
| 106 | 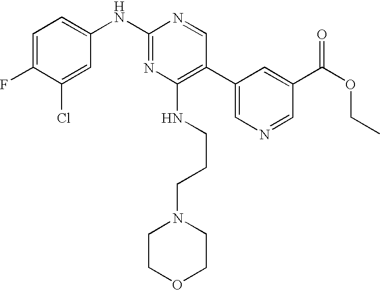 5-[2-(3-Chloro-4- fluoro-phenylamino)-4- (3-morpholin-4-yl- propylamino)- pyrimidin-5-yl]- nicotinic acid ethyl ester | MS(ES): 515 (M + 1) for C₂₅H₂₈ClFN₆O₃. ¹H NMR (400 MHz, DMSO- d₆): δ 1.34 (t, J = 7.08 Hz, 3H), 1.73 (t, J = 6.56 Hz, 2H), 2.29- 2.35 (m, 6H), 3.35-3.46 (m, 6H), 4.37 (q, J = 7.08 Hz, 2H),m 6.96 (t, J = 5.04 Hz, 1H), 7.31 (t, J = 9.12 Hz, 1H), 7.60 (dt, J = 2.96, 6.44 Hz, 1H), 7.85 (s, 1H), 8.22 (t, J = 1.96 Hz, 1H), 8.27 (dd, J = 2.52, 6.88 Hz, 1H), 8.80 (d, J = 2.04 Hz, 1H), 9.05 (d, J = 1.88 Hz, 1H), 9.49 (s, 1H). | 5-Bromo-N²- (3-chloro-4- fluoro- phenyl)-N⁴- (3- morpholin-4- yl-propyl)- pyrimidine- 2,4-diamine (Intermediate 48) |
| 107 | 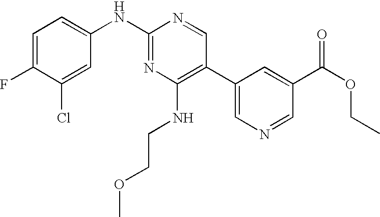 5-[2-(3-Chloro-4- fluoro-phenylamino)-4- (2-methoxy- ethylamino)-pyrimidin- 5-yl]-nicotinic acid ethyl ester | MS(ES): 446 (M + 1) for C₂₁H₂₁ClFN₅O₃. ¹H NMR (400 MHz, DMSO-d₆): δ 1.34 (t, J = 7.12 Hz, 3H), 3.25 (s, 3H), 3.50-3.54 (m, 4H), 4.37 (q, J = 7.08 Hz, 2H), 6.95 (br s, 1H), 7.29 (t, J = 9.12 Hz, 1H), 7.6 (m, 1H), 7.86 (s, 1H), 8.19 (m, 2H), 8.77 (d, J = 2.2 Hz, 1H), 9.04 (d, J = 2.04 Hz, 1H), 9.48 (s, 1H). | 5-Bromo-N²- (3-chloro-4- fluoro- phenyl)-N⁴- (2-methoxy- ethyl)- pyrimidine- 2,4-diamine (Intermediate 50) |
| 108 | 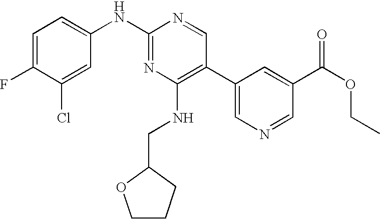 ethyl 5-[2-[(3-chloro-4- fluorophenyl)amino]-4- (oxolan-2- ylmethylamino)pyrimidin- 5-yl]pyridine-3- carboxylate | MS(ES): 472 (M + 1) for C₂₃H₂₃ClFN₅O₃ ¹H NMR (400 MHz, DMSO-d₆): δ 1.34 (t, J = 7.08 Hz, 3H), 1.57-1.63 (m, 1H), 1.76-1.94 (m, 3H), 3.4-3.45 (m, 2H), 3.60- 3.62 (m, 1H), 3.74-3.76 (m, 1H), 4.07-4.1 (m, 1H), 4.37 (q, J = 7.08 Hz, 2H), 6.95 (br s, 1H), 7.3 (t, J = 9.08 Hz, 1H), 7.6 (dd, J = 7.64, 4.88 Hz, 1H), 7.87 (s, 1H), 8.20-8.22 (m, 2H), 8.78 (d, J = 2.2 Hz, 1H), 9.0 (d, J = 2 Hz, 1H), 9.48 (s, 1H). | 5-Bromo-N²- (3-chloro-4- fluoro- phenyl)-N⁴- (tetrahydro- furan-2- ylmethyl)- pyrimidine- 2,4-diamine (Intermediate 75) |
| 109 | 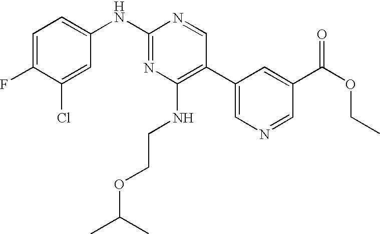 ethyl 5-[2-[(3-chloro-4- fluorophenyl)amino]-4- (2-propan-2- yloxyethylamino)pyrimidin- 5-yl]pyridine-3- carboxylate | MS(ES): 474 (M + 1) for C₂₃H₂₅ClFN₅O₃. ¹H NMR (400 MHz, DMSO- d₆): δ 1.05 (d, J = 6.04 Hz, 6H), 1.34 (t, J = 7.08 Hz, 3H), 3.48- 3.55 (m, 5H), 4.37 (q, J = 5.12 Hz, 2H), 6.87 (d, J = 5.12 Hz, 1H), 7.29 (t, J = 9.08 Hz, 1H), 7.62 (ddd, J = 9.02, 4.12, 2.72 Hz, 1H), 7.86 (s, 1H), 8.18-8.21 (m, 2H), 8.78 (d, J = 2.16 Hz, 1H), 9.05 (d, J = 1.92 Hz, 1H), 9.48 (s, 1H). | 5-Bromo-N²- (3-chloro-4- fluoro- phenyl)-N⁴- (2- isopropoxy- ethyl)- pyrimidine- 2,4-diamine (Intermediate 51) |
| 110 | 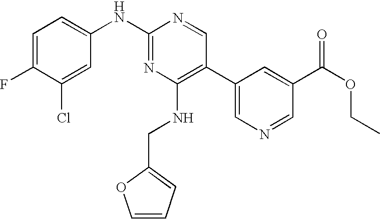 ethyl 5-[2-[(3-chloro-4- fluorophenyl)amino]-4- (furan-2- ylmethylamino)pyrimidin- 5-yl]pyridine-3- carboxylate | MS(ES): 468 (M + 1) for C₂₃H₁₉ClFN₅O₃. ¹H NMR (400 MHz, DMSO- d₆): δ 1.34 (t, J = 7.08 Hz, 3H), 4.37 (q, J = 7.08 Hz, 2H), 4.56 (d, J = 5.72 Hz, 2H), 6.26 (d, J = 3.08 Hz, 1H), 6.37 (dd, J = 3.10, 1.84 Hz, 1H), 7.28 (t, J = 9.08 Hz, 1H), 7.44 (t, J = 5.76 Hz, 1H), 7.55-7.61 (m, 2H), 7.90 (s, 1H), 8.14 (dd, J = 6.84, 2.60 Hz, 1H), 8.23 (t, J = 2.12 Hz, 1H), 8.79 (s, 1H), 9.05 (s, 1H), 9.50 (s, 1H). | 5-Bromo-N²- (3-chloro-4- fluoro- phenyl)-N⁴- (furan-2- ylmethyl)- pyrimidine- 2,4-diamine (Intermediate 52) |
| 111 | 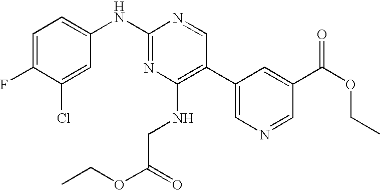 ethyl 5-[2-[(3-chloro-4- fluorophenyl)amino]-4- [(2-ethoxy-2- oxoethyl)amino]pyrimidin- 5-yl]pyridine-3- carboxylate | MS(ES): 474 (M + 1) for C₂₂H₂₁ClFN₅O₄. ¹H NMR (400 MHz MeOD): δ 1.17-1.21 (m, 3H), 1.43 (t, J = 7.12 Hz, 3H), 4.15 (q, J = 7.00 Hz, 4H), 4.45 (q, J = 7.12 Hz, 2H), 7.14 (t, J = 8.96 Hz, 1H), 7.48 (ddd, J = 9.06, 4.12, 2.72 Hz, 1H), 7.88 (s, 1H), 7.94 (dd, J = 6.68, 2.60 Hz, 1H), 8.46 (t, J = 2.08 Hz, 1H), 8.83 (d, J = 2.16 Hz, 1H), 9.12 (d, J = 1.88 Hz, 1H). | [5-Bromo-2- (3-chloro-4- fluoro- phenylamino)- pyrimidin-4- ylamino]- acetic acid ethyl ester (Intermediate 76) |
| 112 | 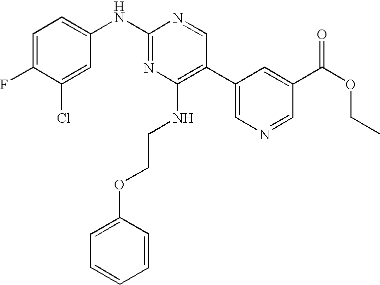 ethyl 5-[2-[(3-chloro-4- fluorophenyl)amino]-4- [(2-ethoxy-2- oxoethyl)amino]pyrimidin- 5-yl]pyridine-3- carboxylate | MS(ES): 508 (M + 1) for C₂₆H₂₃ClFN₅O₃. ¹H NMR (400 MHz DMSO-d₆): δ 1.32 (t, J = 7.12 Hz, 3H), 3.74 (q, J = 5.88 Hz, 2H), 4.16 (t, J = 6.24 Hz, 2H), 4.37 (q, J = 7.12 Hz, 2H), 6.93 (m, 3H), 7.1 (t, J = 5.6 Hz, 1H), 7.25 (m, 3H), 7.62 (ddd, J = 9.08, 4.28, 2.68 Hz, 1H), 7.89 (s, 1H), 8.2 (dd, J = 6.9, 2.64 Hz, 1H), 8.22 (t, J = 2.16 Hz, 1H), 8.77 (d, J = 2.24 Hz, 1H), 9.05 (d, J = 2 Hz, 1H), 9.51 (s, 1H). | 5-Bromo-N²- (3-chloro-4- fluoro- phenyl)-N⁴- (2-phenoxy- ethyl)- pyrimidine- 2,4-diamine (Intermediate 53) |
| 113 | 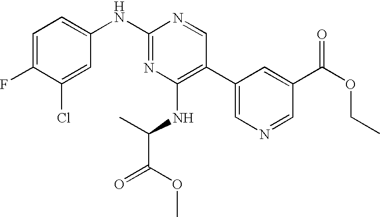 ethyl 5-[2-[(3-chloro-4- fluorophenyl)amino]-4- [[(2R)-1-methoxy-1- oxopropan-2- yl]amino]pyrimidin-5- yl]pyridine-3- carboxylate | MS(ES): 474.1 (M) for C₂₂H₂₁ClFN₅O₄. ¹H NMR (400 MHz DMSO-d₆): δ 1.36 (t, J = 7.08 Hz, 3H), 1.39 (d, J = 7.24 Hz, 3H), 4.37 (q, J = 7.08 Hz, 2H), 4.74-4.78 (m, 1H), 7.17 (d, J = 7.34 Hz, 1H), 7.29 (t, J = 9.12 Hz, 1H), 7.59 (ddd, J = 9.10, 4.22, 2.72 Hz, 1H), 7.94 (s, 1H), 8.02 (dd, J = 6.82, 2.52 Hz, 1H), 8.27 (t, J = 2.16 Hz, 1H), 8.82 (d, J = 2.16 Hz, 1H), 9.05 (d, J = 2 Hz, 1H). | 2-(5-Bromo- 2-chloro- pyrimidin-4- ylamino)- propionic acid methyl ester (Intermediate 54) |
| 114 | 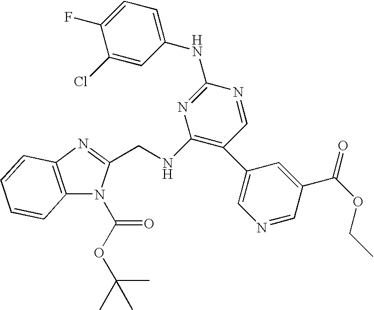 tert-butyl 2-[[[2-[(3- chloro-4- fluorophenyl)amino]-5- (5- ethoxycarbonylpyridin- 3-yl)pyrimidin-4- yl]amino]methyl] benzimidazole- 1-carboxylate | MS(ES): 618.3 (M + 1) for C₃₁H₂₉ClFN₇O₄. ¹H NMR (400 MHz DMSO-d₆): δ 1.32 (t, J = 6.96 Hz, 3H), 1.66 (s, 9H), 4.36 (q, J = 6.92 Hz, 2H), 5.01 (d, J = 4.84 Hz, 2H), 7.09 (t, J = 9.20 Hz, 1H), 7.29- 7.38 (m, 3H), 7.51 (br s, 1H), 7.65 (d, J = 7.56 Hz, 1H), 7.88 (br s, 1H), 7.93 (d, J = 7.76 Hz, 1H), 7.99 (s, 1H), 8.47 (s, 1H), 8.96 (s, 1H), 9.08 (s, 1H), 9.50 (s, 1H). | Tert-butyl 2- [[[5-bromo- 2-[(3-chloro- 4- fluorophenyl) amino] pyrimidin-4- yl]amino] methyl]benz- imidazole-1- carboxylate (Intermediate 126) |
| 115 | 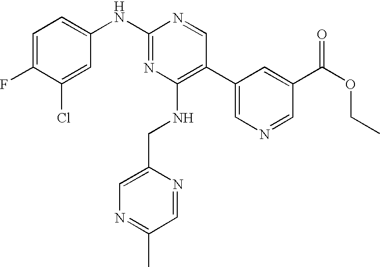 ethyl 5-[2-[(3-chloro-4- fluorophenyl)amino]-4- [(5-methylpyrazin-2- yl)methylamino]pyrimidin- 5-yl]pyridine-3- carboxylate | MS(ES): 494 (M + 1) for C₂₄H₂₁ClFN₇O₂. ¹H NMR (400 MHz DMSO-d₆): δ 1.34 (t, J = 7.08 Hz, 3H), 2.44 (s, 3H), 4.37 (q, J = 7.12 Hz, 2H), 4.67 (d, J = 5.80 Hz, 2H), 7.21 (t, J = 9.08 Hz, 1H), 7.51 (dt, J = 8.59, 4.16 Hz, 1H), 7.59 (t, J = 5.32 Hz, 1H), 7.96 (m, 2H), 8.32 (t, J = 2.12 Hz, 1H), 8.48 (d, J = 8.32 Hz, 2H), 8.87 (d, J = 2.16 Hz, 1H), 9.07 (d, J = 1.96 Hz, 1H), 9.47 (s, 1H). | 5-Bromo-N²- (3-chloro-4- fluoro- phenyl)-N⁴- (5-methyl- pyrazin-2- ylmethyl)- pyrimidine- 2,4-diamine (Intermediate 77) |
| 116 | 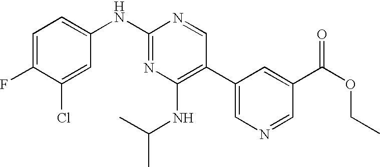 ethyl 5-[2-[(3-chloro-4- fluorophenyl)amino]-4- (propan-2- ylamino)pyrimidin-5- yl]pyridine-3- carboxylate | MS(ES): 430 (M + 1) for C₂₁H₂₁ClFN₅O₂ ¹H NMR (400 MHz DMSO-d₆): δ 1.17 (d, J = 6.56 Hz, 6H), 1.34 (t, J = 7.12 Hz, 3H), 4.37 (m, 3H), 6.62 (d, J = 7.8 Hz, 1H), 7.3 (t, J = 11.28 Hz, 1H), 7.55- 7.59 (m, 1H), 7.84 (s, 1H), 8.2 (t, J = 1.96 Hz, 1H), 8.28 (dd, J = 6.9, 2.48 Hz, 1H), 8.78 (d, J = 1.92 Hz, 1H), 9.04 (d, J = 1.76 Hz, 1H), 9.47 (s, 1H). | (5-Bromo-2- chloro- pyrimidin-4- yl)-isopropyl- amine (Intermediate 56) |
| 117 | 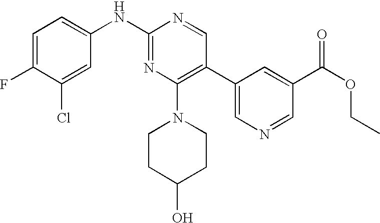 ethyl 5-[2-[(3-chloro-4- fluorophenyl)amino]-4- (4-hydroxypiperidin-1- yl)pyrimidin-5- yl]pyridine-3- carboxylate | MS(ES): 472 (M + 1) for C₂₃H₂₃ClFN₅O₃. ¹H NMR (400 MHz DMSO-d₆): δ 1.32 (m, 2H), 1.35 (t, J = 7.08 Hz, 3H), 1.66-1.69 (m, 2H), 2.95 (t, J = 10.12 Hz, 2H), 3.5- 3.53 (m, 2H), 3.63-3.64 (br s, 1H), 4.37 (q, J = 7.04 Hz, 2H), 4.71 (d, J = 3.60 Hz, 1H), 7.33 (t, J = 9.12 Hz, 1H), 7.60 (ddd, J = 9.08, 4.16, 2.76 Hz, 1H), 8.13 (s, 1H), 8.22 (dd, J = 6.88, 2.60 Hz, 1H), 8.34 (t, J = 2.12 Hz, 1H), 8.91 (d, J = 2.16 Hz, 1H), 9.00 (d, J = 1.96 Hz, 1H), 9.69 (s, 1H). | 1-[5-Bromo- 2-(3-chloro- 4-fluoro- phenylamino)- pyrimidin-4- yl]-piperidin- 4-ol (Intermediate 78) |
| 118 | 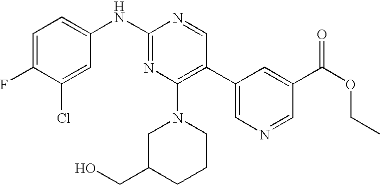 ethyl 5-[2-[(3-chloro-4- fluorophenyl)amino]-4- [3- (hydroxymethyl)piper- idin-1-yl]pyrimidin-5- yl]pyridine-3- carboxylate | MS(ES): 484.2 (M − 1) for C₂₄H₂₅ClFN₅O₃. ¹H NMR (400 MHz DMSO-d₆): δ 1.10-1.13 (m, 1H), 1.36 (t, J = 7.12 Hz, 3H), 1.40 (m, 1H), 1.52-1.69 (m, 3H), 2.60 (t, 1H), 2.76 (t, J = 10.76 Hz, 1H), 3.13 (dd, J = 10.38, 7.44 Hz, 1H), 3.21 (dd, J = 10.56, 5.32 Hz, 1H), 3.58 (d, J = 12.56 Hz, 1H), 3.74 (d, J = 11.48 Hz, 1H), 4.37 (q, J = 7.04 Hz, 2H), 7.32 (t, J = 9.12 Hz, 1H), 7.66 (ddd, J = 9.10, 4.22, 2.76 Hz, 1H), 8.12 (s, 1H), 8.17 (dd, J = 6.86, 2.64 Hz, 1H), 8.31-8.33 (m, 1H), 8.89 (d, J = 2.12 Hz, 1H), 8.99 (d, J = 1.96 Hz, 1H), 9.69 (s, 1H). | {1-[5-Bromo- 2-(3-chloro- 4-fluoro- phenylamino)- pyrimidin-4- yl]-piperidin- 3-yl}- methanol (Intermediate 79) |
| 119 | 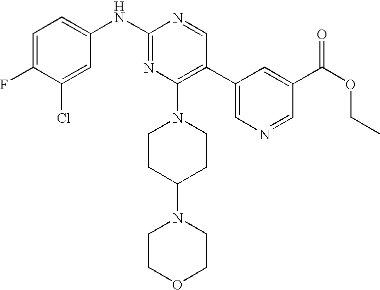 ethyl 5-[2-[(3-chloro-4- fluorophenyl)amino]-4- (4-morpholin-4- ylpiperidin-1- yl)pyrimidin-5- yl]pyridine-3- carboxylate | MS(ES): 541 (M + 1) for C₂₇H₃₀ClFN₆O₃. ¹H NMR (400 MHz, CDCl₃): δ 1.45 (t, J = 7.12 Hz, 3H), 1.9 (m, 2H), 2.40 (br s, 1H), 2.53 (br s, 4H), 2.80 (m, 2H), 3.71 (br s, 4H), 3.85 (d, J = 13.04 Hz, 2H), 4.46 (q, J = 7.12 Hz, 2H), 6.90 (s, 1H), 7.10 (t, J = 8.80 Hz, 1H), 7.27 (m, 1H), 7.98 (s, 1H), 8.06 (d, J = 3.96 Hz, 1H), 8.35 (t, J = 2.12 Hz, 1H), 8.84 (d, J = 2.24 Hz, 1H), 9.16 (d, J = 1.96 Hz, 1H). | [5-Bromo-4- (4- morpholin-4- yl-piperidin- 1-yl)- pyrimidin-2- yl]-(3-chloro- 4-fluoro- phenyl)- amine (Intermediate 80) |
| 120 | 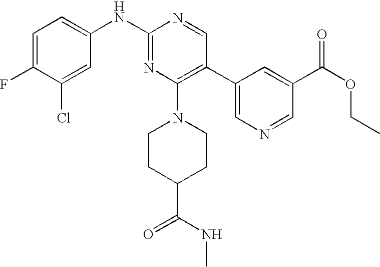 ethyl 5-[2-[(3-chloro-4- fluorophenyl)amino]-4- [4- (methylcarbamoyl)piper- idin-1-yl]pyrimidin-5- yl]pyridine-3- carboxylate | MS(ES): 513 (M + 1) for C₂₅H₂₆ClFN₆O₃ ¹H NMR (400 MHz DMSO- d₆): δ 1.35 (t, J = 7.12 Hz, 3H), 1.5 (m, 4H), 2.26 (m, 1H), 2.52 (d, J = 4.56 Hz, 3H), 2.79 (t, J = 11.32 Hz, 2H), 3.84 (d, J = 13.04 Hz, 2H), 4.36 (q, J = 7.08 Hz, 2H), 7.33 (t, J = 9.08 Hz, 1H), 7.6 (ddd, J = 9.06, 4.22, 2.68 Hz, 1H), 7.70 (q, J = 4.24 Hz, 1H), 8.1 (s, 1H), 8.21 (dd, J = 6.92, 2.64 Hz, 1H), 8.34 (t, J = 2.16 Hz, 1H), 8.91 (d, J = 2.2 Hz, 1H), 9.0 (d, J = 2 Hz, 1H), 9.71 (s, 1H). | 1-[5-Bromo- 2-(3-chloro- 4-fluoro- phenylamino)- pyrimidin-4- yl]- piperidine-4- carboxylic acid methylamide (Intermediate 81) |
| 121 | 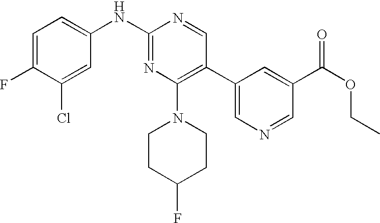 ethyl 5-[2-[(3-chloro-4- fluorophenyl)amino]-4- (4-fluoropiperidin-1- yl)pyrimidin-5- yl]pyridine-3- carboxylate | MS(ES): 474 (M + 1) for C₂₃H₂₂ClF₂N₅O₂. ¹H NMR (400 MHz, MeOD): δ 1.43 (t, J = 7.0 Hz, 3H), 1.77 (m, 2H), 1.9 (m, 2H), 3.3 (m, 2H), 3.45 (m, 2H), 4.45 (q, J = 7.16 Hz, 2H), 7.17 (t, J = 8.96 Hz, 1H), 7.49 (ddd, J = 8.96, 4.08, 2.68 Hz, 1H), 8.07 (s, 1H), 8.11 (dd, J = 6.76, 2.64 Hz, 1H), 8.49 (t, J = 2.08 Hz, 1H) 8.87 (d, J = 2.16 Hz, 1H), 9.06 (d, J = 1.92 Hz, 1H). | [5-Bromo-4- (4-fluoro- piperidin-1- yl)- pyrimidin-2- yl]-(3-chloro- 4-fluoro- phenyl)- amine (Intermediate 82) |
| 122 | 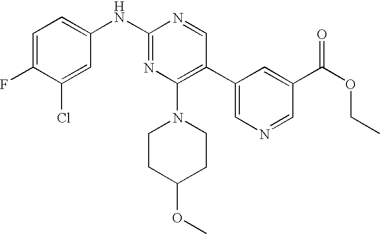 ethyl 5-[2-[3-chloro-4- fluorophenyl)amino]-4- (4-methoxypiperidin-1- yl)pyrimidin-5- yl]pyridine-3- carboxylate | MS(ES): 486 (M + 1) for C₂₄H₂₅ClFN₅O₃. ¹H NMR (400 MHz DMSO-d₆): δ 1.32-1.42 (m, 5H), 1.77-1.79 (m, 2H), 2.99 (t, J = 9.88 Hz, 2H), 3.2 (s, 3H), 3.37 (m, 1H), 3.47 (m, 2H), 4.36 (q, J = 6.96 Hz, 2H), 7.33 (t, J = 9.12 Hz, 1H), 7.58-7.62 (m, 1H), 8.14 (s, 1H), 8.2 (dd, J = 6.9, 2.52 Hz, 1H), 8.35 (t, J = 2 Hz, 1H), 8.91 (d, J = 2.12 Hz, 1H), 9 (d, J = 1.84 Hz, 1H), 9.7 (s, 1H). | [5-Bromo-4- (4-methoxy- piperidin-1- yl)- pyrimidin-2- yl]-(3-chloro- 4-fluoro- phenyl)- amine (Intermediate 83) |
| 123 | 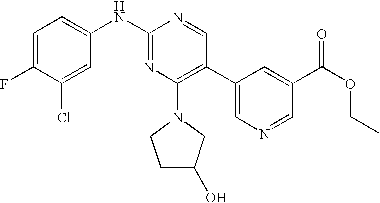 ethyl 5-[2-[(3-chloro-4- fluorophenyl)amino]-4- (3-hydroxypyrrolidin- 1-yl)pyrimidin-5- yl]pyridine-3- carboxylate | MS(ES): 458.2 (M + 1) for C₂₂H₂₁ClFN₅O₃. ¹H NMR (400 MHz DMSO-d₆): δ 1.34 (t, J = 4.60 Hz, 3H), 1.74 (m, 1H), 1.81 (m, 1H), 2.89 (br s, 1H), 3.20 (br s, 2H), 3.41 (br s, 1H), 4.19 (br s, 1H), 4.35 (q, J = 7.04 Hz, 2H), 4.89 (d, J = 3.36 Hz, 1H), 7.32 (t, J = 9.12 Hz, 1H), 7.65 (ddd, J = 9.08, 4.22, 2.68 Hz, 1H), 7.95 (s, 1H), 8.13 (br s, 1H), 8.25 (dd, J = 6.94, 2.60 Hz, 1H), 8.76 (br s, 1H), 9.01 (d, J = 2.00 Hz, 1H), 9.56 (s, 1H). | 1-[5-Bromo- 2-(3-chloro- 4-fluoro- phenylamino)- pyrimidin-4- yl]- pyrrolidin-3- ol (Intermediate 84) |
| 124 | 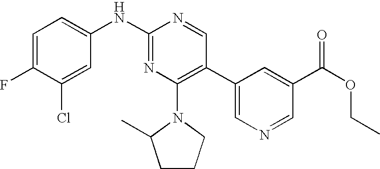 ethyl 5-[2-[(3-chloro-4- fluorophenyl)amino]-4- (2-methylpyrrolidin-1- yl)pyrimidin-5- yl]pyridine-3- carboxylate | MS(ES): 456.2 (M) for C₂₃H₂₃ClFN₅O₂. ¹H NMR (400 MHz MeOD): δ 1.34 (d, J = 6.20 Hz, 3H), 1.43 (t, J = 7.16 Hz, 3H), 1.62-1.66 (m, 2H), 1.9-1.94 (m, 1H), 2.1- 2.14 (m, 1H), 2.8-2.97 (m, 1H), 2.98-3.1 (m, 1H), 4.41-4.48 (s, 1H), 4.45 (q, J = 7.12 Hz, 2H), 7.24 (t, J = 8.96 Hz, 1H), 7.46 (ddd, J = 8.96, 4.06, 2.68 Hz, 1H), 7.88 (s, 1H), 8.09 (dd, J = 6.70, 2.68 Hz, 1H), 8.35 (t, J = 2.00 Hz, 1H), 8.77 (d, J = 1.68 Hz, 1H), 9.12 (d, J = 1.56 Hz, 1H). | [5-Bromo-4- (2-methyl- pyrrolidin-1- yl)- pyrimidin-2- yl]-(3-chloro- 4-fluoro- phenyl)- amine (Intermediate 85) |
| 125 | 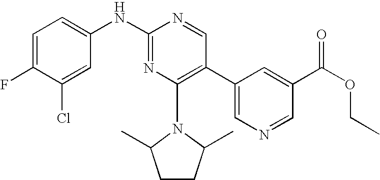 ethyl 5-[2-[(3-chloro-4- fluorophenyl)amino]-4- (2,5- dimethylpyrrolidin-1- yl)pyrimidin-5- yl]pyridine-3- carboxylate | MS(ES): 470 (M + 1) for C₂₄H₂₅ClFN₅O₂. ¹H NMR (400 MHz DMSO-d₆): δ 0.99 (d, J = 6.20 Hz, 6H), 1.34 (t, J = 7.08 Hz, 3H), 1.62 (br s, 2H), 1.95 (br s, 2H), 3.96 (br s, 2H), 4.36 (q, J = 7.08 Hz, 2H), 7.31 (t, J = 9.12 Hz, 1H), 7.56 (dt, J = 8.57, 4.00 Hz, 1H), 7.82 (s, 1H), 8.21 (t, J = 2.04 Hz, 1H), 8.28 (dd, J = 6.88, 2.52 Hz, 1H), 8.80 (d, J = 2.12 Hz, 1H), 9.03 (d, J = 1.92 Hz, 1H), 9.51 (s, 1H). | [5-Bromo-4- (2,5- dimethyl- pyrrolidin-1- yl)- pyrimidin-2- yl]-(3-chloro- 4-fluoro- phenyl)- amine (Intermediate 86) |
| 126 | 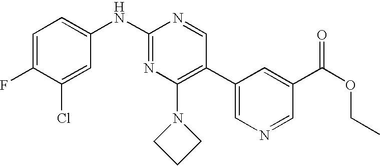 ethyl 5-[4-(azetidin-1- yl)-2-[(3-chloro-4- fluorophenyl)amino] pyrimidin-5-yl]pyridine- 3-carboxylate | MS(ES): 428 (M + 1) for C₂₁H₁₉ClFN₅O₂. ¹H NMR (400 MHz DMSO-d₆): δ 1.35 (t, J = 7.04 Hz, 3H), 2.13-2.21 (m, 2H), 3.76 (br s, 4H), 4.37 (q, J = 7.04 Hz, 2H), 7.31 (t, J = 9.12 Hz, 1H), 7.65 (ddd, J = 9.02, 4.26, 2.64 Hz, 1H), 7.98 (s, 1H), 8.16 (t, J = 2.08 Hz, 1H), 8.25 (dd, J = 6.94, 2.60 Hz, 1H), 8.79 (d, J = 2.16 Hz, 1H), 9.02 (d, J = 1.96 Hz, 1H), 9.61 (s, 1H). | (4-Azetidin- 1-yl-5- bromo- pyrimidin-2- yl)-(3-chloro- 4-fluoro- phenyl)- amine (Intermediate 87) |
| 127 | 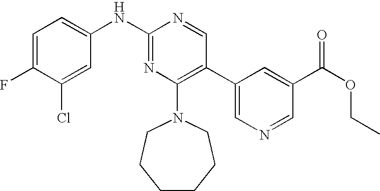 ethyl 5-[4-(azepan-1- yl)-2-[(3-chloro-4- fluorophenyl)amino] pyrimidin-5-yl]pyridine- 3-carboxylate | MS(ES): 470 (M + 1) for C₂₄H₂₅ClFN₅O₂ ¹H NMR (400 MHz, DMSO- d₆): δ 1.34 (t, J = 7.04 Hz, 3H), 1.41 (m, 4H), 1.60 (m, 4H), 3.30 (m, 4H), 4.36 (q, J = 7.08 Hz, 2H), 7.32 (t, J = 9.12 Hz, 1H), 7.54-7.57 (m, 1H), 7.91 (s, 1H), 8.15 (t, J = 2.12 Hz, 1H), 8.26 (dd, J = 6.88, 2.44 Hz, 1H), 8.78 (d, J = 2.16 Hz, 1H), 9.00 (d, J = 1.96 Hz, 1H), 9.57 (s, 1H). | (4-Azepan-1- yl-5-bromo- pyrimidin-2- yl)-(3-chloro- 4-fluoro- phenyl)- amine (Intermediate 88) |
| 128 | 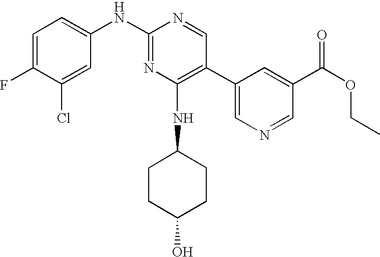 ethyl 5-[2-[(3-chloro-4- fluorophenyl)amino]-4- [(trans-4- hydroxycyclohexyl) amino]pyrimidin-5- yl]pyridine-3- carboxylate | MS(ES): 486 (M + 1) for C₂₄H₂₅ClFN₅O₃. ¹H NMR (400 MHz, DMSO- d₆): δ 1.30 (m, 4H), 1.33 (t, J = 7.00 Hz, 3H), 1.84 (m, 4H), 3.98 (s, 1H), 4.36 (q, J = 7.04 Hz, 2H), 4.55 (d, J = 3.28 Hz, 1H), 6.58 (d, J = 7.84 Hz, 1H), 7.29 (t, J = 9.16 Hz, 1H), 7.54 (dd, J = 5.60, 2.52 Hz, 1H), 7.83 (s, 1H), 8.19 (d, J = 1.92 Hz, 1H), 8.28 (dd, J = 9.30, 4.52 Hz, 1H), 8.76 (d, J = 1.88 Hz, 1H), 9.02 (d, J = 1.76 Hz, 1H), 9.49 (s, 1H). | Trans-4-[5- Bromo-2-(3- chloro-4- fluoro- phenylamino)- pyrimidin-4- ylamino]- cyclohexanol (Intermediate 89) |
| 129 | 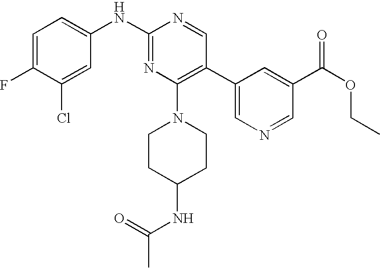 ethyl 5-[4-(4- acetamidopiperidin-1- yl)-2-[(3-chloro-4- fluorophenyl)amino] pyrimidin-5-yl]pyridine- 3-carboxylate | MS(ES): 513 (M + 1) for C₂₅H₂₆ClFN₆O₃. ¹H NMR (400 MHz, DMSO- d₆): δ 1.29 (m, 2H), 1.35 (t, J = 7.08 Hz, 3H), 1.66 (m, 2H), 1.74 (s, 3H), 2.90 (m, 2H), 3.60 (m, 2H), 3.70 (br s, 1H), 4.37 (q, J = 7.08 Hz, 2H), 7.34 (t, J = 9.12 Hz, 1H), 7.59-7.62 (m, 1H), 7.81 (d, J = 7.60 Hz, 1H), 8.13 (s, 1H), 8.21 (dd, J = 6.90, 2.64 Hz, 1H), 8.32 (t, J = 2.12 Hz, 1H), 8.91 (d, J = 2.20 Hz, 1H), 9.00 (d, J = 2.00 Hz, 1H), 9.72 (s, 1H). | N-{1-[5- Bromo-2-(3- chloro-4- fluoro- phenylamino)- pyrimidin-4- yl]-piperidin- 4-yl}- acetamide (Intermediate 90) |
| 130 | 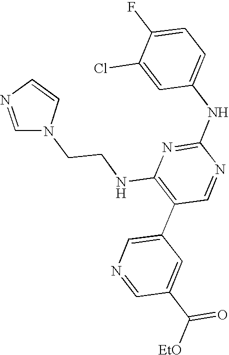 ethyl 5-(2-[(3-chloro-4- fluorophenyl)amino]-4- {[2-(1H-imidazol-1- yl)ethyl]amino}pyrimidin- 5-yl)pyridine-3- carboxylate | MS(ES): 482 (M + 1) for C₂₃H₂₁ClFN₇O₂ ¹H NMR (400 MHz, DMSO- d₆): δ 1.36 (t, J = 7.08 Hz, 3H), 3.65 (q, J = 5.72 Hz, 2H), 4.22 (t, J = 5.88 Hz, 2H), 4.39 (q, J = 6.96 Hz, 2H), 6.87 (s, 1H), 7.00 (t, J = 5.08 Hz, 1H), 7.14 (s, 1H), 7.32 (t, J = 9.08 Hz, 1H), 7.59 (s, 1H), 7.61-7.64 (m, 1H), 7.89 (s, 1H), 8.14-8.17 (m, 2H), 8.31 (s, 1H), 8.72 (d, J = 1.76 Hz, 1H), 9.05 (s, 1H), 9.50 (s, 1H). | 5-bromo-N²- (3-chloro-4- fluorophenyl)- N⁴-[2-(1H- imidazol-1- yl)ethyl] pyrimidine- 2,4- diamine (Intermediate 64) |
| 131 | 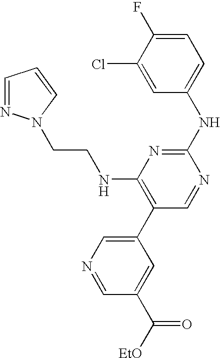 ethyl 5-(2-[(3-chloro-4- fluorophenyl)amino]-4- {[2-(1H-pyrazol-1- yl)ethyl]amino}pyrimidin- 5-yl)pyridine-3- carboxylate | MS(ES): 482 (M + 1) for C₂₃H₂₁ClFN₇O₂. ¹H NMR (400 MHz, DMSO- d₆): δ 1.34 (t, J = 7.04 Hz, 3H), 3.72 (q, J = 5.80 Hz, 2H), 4.35- 4.40 (m, 4H), 6.22 (t, J = 2.08 Hz, 1H), 6.96 (t, J = 5.36 Hz, 1H), 7.31 (t, J = 9.12 Hz, 1H), 7.43 (t, J = 1.28 Hz, 1H), 7.68- 7.72 (m, 2H), 7.88 (s, 1H), 8.13 (dd, J = 2.56, 6.86 Hz, 1H), 8.17 (t, J = 2.08 Hz, 1H), 8.72 (d, J = 2.16 Hz, 1H), 9.03 (d, J = 1.92 Hz, 1H), 9.51 (s, 1H). | 5-bromo-N²- (3-chloro-4- fluorophenyl)- N⁴-[2-(1H- pyrazol-1- yl)ethyl] pyrimidine- 2,4- diamine (Intermediate 91) |
| 132 | 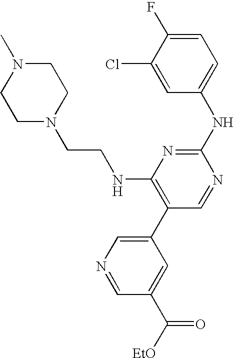 ethyl 5-(2-[(3-chloro-4- fluorophenyl)amino]-4- {[2-(4- methylpiperazin-1- yl)ethyl]amino}pyrimidin- 5-yl)pyridine-3- carboxylate | MS(ES): 514 (M + 1) for C₂₅H₂₉ClFN₇O₂ ¹H NMR (400 MHz, DMSO- d₆): δ 1.34 (t, J = 7.08 Hz, 3H), 2.14 (s, 3H), 2.30-2.50 (m, 8H), 3.40 (m, 2H), 3.50 (m, 2H), 4.38 (q, J = 7.12 Hz, 2H), 6.77 (m, 1H), 7.29 (t, J = 9.08 Hz, 1H), 7.65 (ddd, J = 2.64, 4.24, 9.09 Hz, 1H), 7.88 (s, 1H), 8.19 (dd, J = 2.60, 6.90 Hz, 1H), 8.23- 8.24 (m, 1H), 8.81 (d, J = 2.20 Hz, 1H), 9.06 (d, J = 2.00 Hz, 1H), 9.48 (s, 1H). | 5-bromo-N²- (3-chloro-4- fluorophenyl)- N⁴-[2-(4- methylpiper- azin-1- yl)ethyl] pyrimidine- 2,4- diamine (Intermediate 92) |
| 133 | 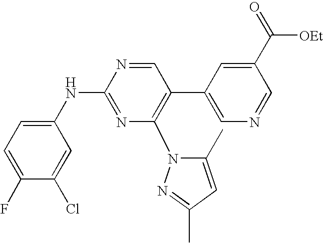 ethyl 5-{2-[(3-chloro- 4-fluorophenyl)amino]- 4-(3,5-dimethyl-1H- pyrazol-1-yl)pyrimidin- 5-yl}pyridine-3- carboxylate | MS(ES): 467 (M + 1) for C₂₃H₂₀ClFN₆O₂. ¹H NMR (400 MHz, DMSO- d₆): δ 1.31 (t, J = 7.08 Hz, 3H), 1.91 (s, 3H), 2.37 (s, 3H), 4.33 (q, J = 7.12 Hz, 2H), 6.09 (s, 1H), 7.41 (t, J = 9.08 Hz, 1H), 7.62-7.66 (m, 1H), 7.86 (t, J = 2.12 Hz, 1H), 8.09 (dd, J = 2.56, 6.68 Hz, 1H), 8.49 (d, J = 2.24 Hz, 1H), 8.86 (s, 1H), 8.95 (d, J = 1.96 Hz, 1H), 10.30 (s, 1H). | 5-bromo-N- (3-chloro-4- fluorophenyl)- 4-(3,5- dimethyl-1H- pyrazol-1- yl)pyrimidin- 2-amine (Intermediate 112) |
| 134 | 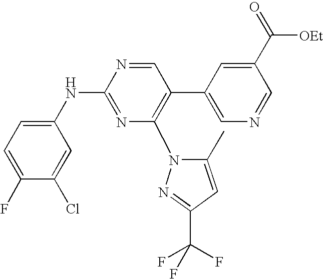 ethyl 5-{2-[(3-chloro- 4-fluorophenyl)amino]- 4-[5-methyl-3- (trifluoromethyl)-1H- pyrazol-1-yl]pyrimidin- 5-yl}pyridine-3- carboxylate | MS(ES): 521 (M + 1) for C₂₃H₁₇ClF₄N₆O₂. ¹H NMR (400 MHz, DMSO- d₆): δ 1.30 (t, J = 7.20 Hz, 3H), 2.43 (s, 3H), 4.31 (q, J = 8.40 Hz, 2H), 6.79 (s, 1H), 7.43 (t, J = 8.80 Hz, 1H), 7.65-7.69 (m, 1H), 7.79-7.80 (m, 1H), 8.07- 8.08 (m, 1H), 8.61 (d, J = 2.40 Hz, 1H), 8.98-9.02 (m, 2H), 10.51 (s, 1H). | 5-bromo-N- (3-chloro-4- fluorophenyl)- 4-[5-methyl- 3- (trifluoro- methyl)-1H- pyrazol-1- yl]pyrimidin- 2-amine (Intermediate 113) |
| 135 | 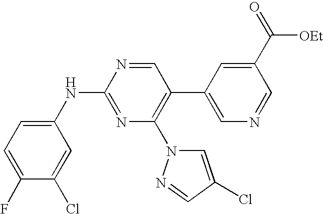 ethyl 5-{2-[(3-chloro- 4-fluorophenyl)amino]- 4-(4-chloro-1H- pyrazol-1-yl)pyrimidin- 5-yl}pyridine-3- carboxylate | MS(ES): 473 (M) and 475 (M + 2) for C₂₁H₁₅Cl₂FN₆O₂. ¹H NMR (300 MHz, DMSO- d₆): δ 1.32 (t, J = 7.02 Hz, 3H), 4.34 (q, J = 7.02 Hz, 2H), 7.43 (t, J = 8.88 Hz, 1H), 7.71-7.73 (m, 1H), 7.74 (s, 1H), 8.03 (d, J = 4.53 Hz, 1H), 8.11 (s, 1H), 8.59 (s, 1H), 8.66 (d, J = 1.98 Hz, 1H), 8.72 (s, 1H), 9.01 (d, J = 1.62 Hz, 1H). | 5-bromo-N- (3-chloro-4- fluorophenyl)- 4-(4-chloro- 1H-pyrazol- 1- yl)pyrimidin- 2-amine (Intermediate 114) |
| 136 | 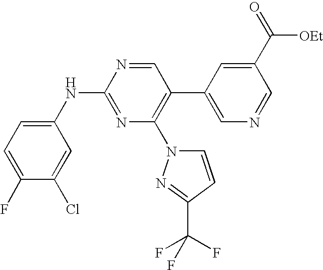 ethyl 5-{2-[(3-chloro- 4-fluorophenyl)amino]- 4-[3-(trifluoromethyl)- 1H-pyrazol-1- yl]pyrimidin-5- yl}pyridine-3- carboxylate | MS(ES): 507 (M + 1) for C₂₂H₁₅ClF₄N₆O₂. ¹H NMR (400 MHz DMSO-d₆): δ 1.30 (t, J = 7.0 Hz, 3H), 4.33 (q, J = 6.92 Hz, 2H), 7.05 (s, 1H), 7.43 (t, J = 6.92 Hz 1H), 7.71-7.74 (m, 1H), 8.03 (s, 1H), 8.03-8.07 (m, 1H), 8.55 (s, 1H), 8.68 (m, 1H), 8.85 (s, 1H), 9.02 (s, 1H), 10.48 (s, 1H). | 5-bromo-N- (3-chloro-4- fluorophenyl)- 4-[3- (trifluoro- methyl)-1H- pyrazol-1- yl]pyrimidin- 2-amine (Intermediate 115) |
| 137 | 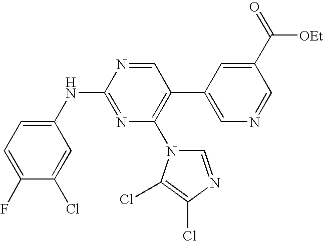 ethyl 5-{2-[(3-chloro- 4-fluorophenyl)amino]- 4-(4,5-dichloro-1H- imidazol-1- yl)pyrimidin-5- yl}pyridine-3- carboxylate | MS(ES): 507 (M) and 509 (M + 2) for C₂₁H₁₄Cl₃FN₆O. ¹H NMR (400 MHz DMSO-d₆): δ 1.32 (t, J = 7.08 Hz, 3H), 4.33 (q, J = 7.12 Hz, 2H), 7.43 (t, J = 9.08 Hz, 1H), 7.69 (ddd, J = 9.08, 4.24, 2.76 Hz, 1H), 7.95 (m, 1H), 8.05 (d, J = 4.28 Hz, 1H), 8.12 (s, 1H), 8.67 (d, J = 2.16 Hz, 1H), 9.04 (d, J = 1.92 Hz, 1H), 9.07 (s, 1H). | 5-bromo-N- (3-chloro-4- fluorophenyl)- 4-(4,5- dichloro-1H- imidazol-1- yl)pyrimidin- 2-amine (Intermediate 116) |
| 138 | 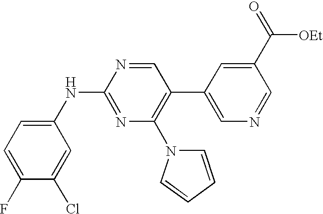 ethyl 5-{2-[(3-chloro- 4-fluorophenyl)amino]- 4-(1H-pyrrol-1- yl)pyrimidin-5- yl}pyridine-3- carboxylate | MS(ES): 438 (M + 1) for C₂₂H₁₇ClFN₅O₂. ¹H NMR (300 MHz, DMSO- d₆): δ 1.31 (t, J = 6.96 Hz, 3H), 4.33 (q, J = 7.08 Hz, 2H), 6.19 (s, 2H), 6.90 (s, 2H), 7.40 (t, 1H), 7.69-7.73 (m, 1H), 8.10- 8.14 (m, 2H), 8.64-8.66 (m, 2H), 9.04 (s, 1H), 10.25 (s, 1H). | 5-bromo-N- (3-chloro-4- fluorophenyl)- 4-(1H- pyrrol-1- yl)pyrimidin- 2-amine (Intermediate 117) |
| 139 | 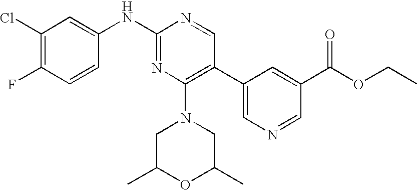 ethyl 5-(2-(3-chloro-4- fluorophenylamino)-4- (2,6- dimethylmorpholino) pyrimidin-5-yl)nicotinate | MS(ES): 486 (M + 1) for C₂₄H₂₅ClFN₅O₃ ¹H NMR (300 MHz, DMSO- d6) δ ppm 0.96 (d, J = 6.03 Hz, 6 H) 1.34 (t, J = 7.06 Hz, 3 H) 3.25- 3.41 (m, 2 H) 3.40-3.70 (m, 4 H) 4.37 (q, J = 7.10 Hz, 2 H) 7.33 (t, J = 9.14 Hz, 1 H) 7.54 (d, J = 13.00 Hz, 1 H) 8.05-8.28 (m, 2 H) 8.34 (t, J = 2.17 Hz, 1 H) 8.91 (d, J = 2.26 Hz, 1 H) 9.01 (d, J = 1.88 Hz, 1 H) 9.75 (s, 1 H) | 5-bromo-N- (3-chloro-4- fluorophenyl)- 4-(2,6- dimethyl- morpholino) pyrimidin-2- amine (Intermediate 95) |
| 140 | 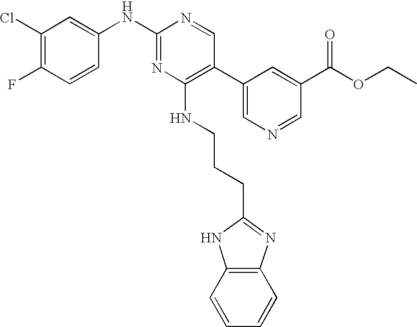 ethyl 5-(4-(3-(1H- benzo[d]imidazol-2- yl)propylamino)-2-(3- chloro-4- fluorophenylamino) pyrimidin-5-yl)nicotinate | MS(ES): (M + 1) for C₂₈H₂₅ClFN₇O₂ ¹H NMR (300 MHz, DMSO- d6) δ ppm 1.30 (t, J = 7.16 Hz, 3 H) 1.98-2.19 (m, J = 1.51 Hz, 2 H) 2.76-2.97 (m, 2 H) 3.37- 3.57 (m, 2 H) 4.34 (q, J = 7.03 Hz, 2 H) 6.95-7.18 (m, 3 H) 7.26 (t, J = 9.14 Hz, 1 H) 7.30- 7.49 (m, 2 H) 7.51-7.71 (m, 1 H) 7.86 (s, 1H) 8.10-8.36 (m, 2 H) 8.80 (d, J = 2.07 Hz, 1H) 9.03 (d, J = 2.07 Hz, 1 H) 9.49 (s, 1 H) 12.15 (s, 1 H) | N⁴-(3-(1H- benzo[d]imi- dazol-2- yl)propyl)-5- bromo-N²-(3- chloro-4- fluorophenyl) pyrimidine- 2,4-diamine (Intermediate 93) |
| 141 | 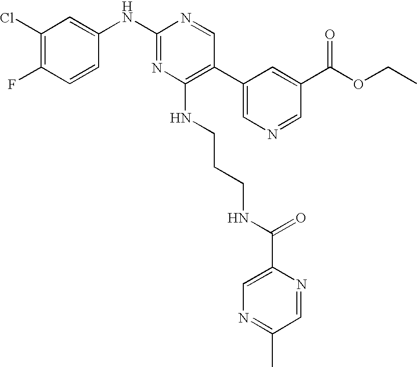 ethyl 5-(2-(3-chloro-4- fluorophenylamino)-4- (3-(5-methylpyrazine- 2- carboxamido)propyl- amino)pyrimidin-5- yl)nicotinate | MS(ES): 565 (M + 1) for C₂₇H₂₆ClFN₈O₃ ¹H NMR (300 MHz, DMSO- d6) δ ppm 1.32 (t, J = 7.06 Hz, 3 H) 1.74-1.93 (m, 2 H) 2.56 (s, 3 H) 3.32-3.61 (m, 4 H) 4.35 (q, J = 7.10 Hz, 2 H) 7.01 (t, J = 5.18 Hz, 1 H) 7.28 (t, J = 9.14 Hz, 1 H) 7.50-7.72 (m, 1 H) 7.86 (s, 1 H) 8.10-8.38 (m, 2 H) 8.54 (s, 1 H) 8.81 (d, J = 2.07 Hz, 1 H) 8.91 (t, J = 6.03 Hz, 1 H) 8.98 (d, J = 1.13 Hz, 1 H) 9.05 (d, J = 2.07 Hz, 1 H) 9.48 (s, 1 H) | N-(3-(5- bromo-2-(3- chloro-4- fluorophenyl amino) pyrimidin-4- ylamino) propyl)-5- methyl- pyrazine-2- carboxamide (Intermediate 102) |
| 142 | 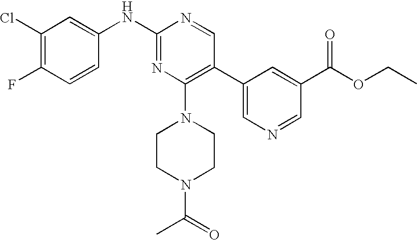 ethyl 5-(4-(4- acetylpiperazin-1-yl)-2- (3-chloro-4- fluorophenylamino) pyrimidin-5-yl)nicotinate | MS(ES): 499 (M + 1) for C₂₄H₂₄ClFN₆O₃ ¹H NMR (300 MHz, DMSO- d6) δ ppm 1.33 (t, J = 7.06 Hz, 3 H) 1.95 (s, 3 H) 3.24 (d, J = 10.93 Hz, 4 H) 3.42 (d, J = 11.49 Hz, 4 H) 4.37 (q, J = 6.97 Hz, 2 H) 7.34 (t, J = 9.14 Hz, 1 H) 7.49-7.79 (m, 1 H) 7.99-8.28 (m, 2 H) 8.28-8.46 (m, 1 H) 8.92 (d, J = 2.07 Hz, 1 H) 9.01 (d, J = 1.88 Hz, 1 H) 9.75 (s, 1 H) | 1-{4-[5- bromo-2-(3- chloro-4- fluorophenyl amino) pyrimidin-4- yl]piperazin- 1- yl}ethanone (Intermediate 98) |
| 143 | 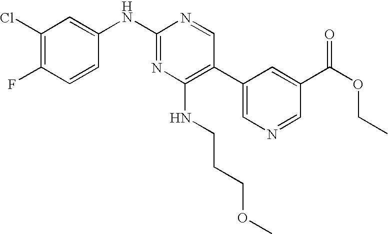 ethyl 5-(2-(3-chloro-4- fluorophenylamino)-4- (3- methoxypropylamino) pyrimidin-5- yl)nicotinate | MS(ES): 460 (M + 1) for C₂₂H₂₃ClFN₅O₃ ¹H NMR (300 MHz, DMSO- d6) δ ppm 1.34 (t, J = 7.06 Hz, 3 H) 1.67-1.88 (m, 2 H) 3.18 (s, 3 H) 3.25-3.50 (m, 4 H) 4.38 (q, J = 7.16 Hz, 2 H) 7.39 (t, J = 9.04 Hz, 1 H) 7.47-7.63 (m, 1 H) 7.73 (s, 1 H) 7.89 (s, 1 H) 8.07 (d, J = 4.90 Hz, 1 H) 8.25 (t, J = 2.07 Hz, 1 H) 8.80 (d, J = 2.26 Hz, 1 H) 9.11 (d, J = 1.88 Hz, 1 H) 9.98 (s, 1 H) | 5-bromo-N²- (3-chloro-4- fluorophenyl)- N⁴-(3- methoxy- propyl) pyrimidine- 2,4-diamine (Intermediate 119) |
| 144 | 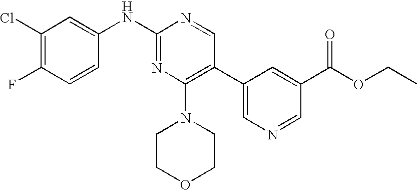 ethyl 5-(2-(3-chloro-4- fluorophenylamino)-4- morpholinopyrimidin- 5-yl)nicotinate | MS(ES): 458 (M + 1) for C₂₂H₂₁ClFN₅O₃ ¹H NMR (300 MHz, DMSO- d6) δ ppm 1.35 (t, J = 7.16 Hz, 3 H) 3.06-3.41 (m, 4 H) 3.45- 3.69 (m, 4 H) 4.31-4.42 (q, 2 H) 7.36 (t, J = 9.14 Hz, 1 H) 7.48- 7.70 (m, 1 H) 8.08 (dd, J = 6.88, 2.54 Hz, 1 H) 8.17 (s, 1H) 8.38 (t, J = 1.98 Hz, 1 H) 8.92 (d, J = 2.07 Hz, 1 H) 9.02 (d, J = 1.88 Hz, 1 H) 9.88 (s, 1 H) | 5-Bromo-N- (3-chloro-4- fluorophenyl)- 4- morpholin-4- ylpyrimidin- 2-amine (Intermediate 111) |
| 145 | 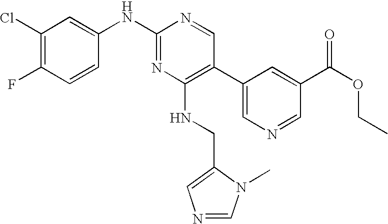 ethyl 5-(2-(3-chloro-4- fluorophenylamino)-4- ((1-methyl-1H- imidazol-5- yl)methylamino) pyrimidin-5-yl) nicotinate | MS(ES): 482 (M + 1) for C₂₃H₂₁ClFN₇O₂ ¹H NMR (300 MHz, DMSO- d6) δ ppm 1.33 (t, J = 7.06 Hz, 3 H) 3.59 (s, 3 H) 4.36 (q, J = 7.16 Hz, 2 H) 4.52 (d, J = 5.09 Hz, 2 H) 6.81 (s, 1 H) 7.20-7.40 (m, 2 H) 7.51 (s, 1 H) 7.56-7.73 (m, 1 H) 7.90 (s, 1 H) 8.13 (dd, J = 6.88, 2.73 Hz, 1 H) 8.22 (t, J = 2.07 Hz, 1 H) 8.78 (d, J = 2.07 Hz, 1 H) 9.04 (d, J = 2.07 Hz, 1 H) 9.49 (s, 1 H) | 5-bromo-N²- (3-chloro-4- fluorophenyl)- N⁴-((1- methyl-1H- imidazol-5- yl)methyl) pyrimidine-2,4- diamine (Intermediate 105) |
| 146 | 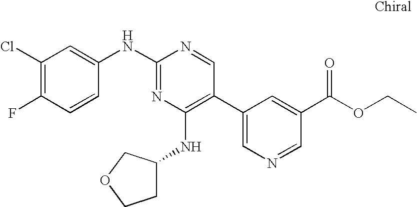 (R)-ethyl 5-(2-(3- chloro-4- fluorophenylamino)-4- (tetrahydrofuran-3- ylamino)pyrimidin-5- yl)nicotinate | MS(ES): 487 (M + 1) for C₂₄H₂₄ClFN₄O₄ ¹H NMR (300 MHz, DMSO- d6) δ ppm 1.33 (t, J = 7.06 Hz, 3 H) 1.78-2.05 (m, 1 H) 2.03- 2.34 (m, 1 H) 3.59 (dd, J = 8.76, 4.43 Hz, 1 H) 3.64-3.87 (m, 2 H) 3.93 (dd, J = 8.85, 6.22 Hz, 1 H) 4.37 (q, J = 7.10 Hz, 2 H) 4.51- 4.77 (m, 1 H) 6.93 (d, J = 6.03 Hz, 1 H) 7.32 (t, J = 9.14 Hz, 1 H) 7.47-7.70 (m, 1 H) 7.88 (s, 1 H) 8.11-8.37 (m, 2 H) 8.78 (d, J = 2.26 Hz, 1 H) 9.03 (d, J = 2.07 Hz, 1 H) 9.53 (s, 1H) | (R)-5-bromo- N²-(3-chloro- 4- fluorophenyl)- N⁴- (tetrahydro- furan-3- yl)pyrimidine- 2,4-diamine (Intermediate 103) |
| 147 | 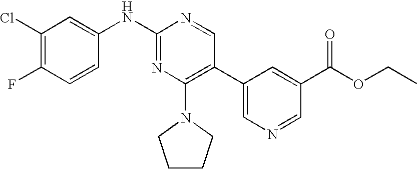 ethyl 5-(2-(3-chloro-4- fluorophenylamino)-4- (pyrrolidin-1- yl)pyrimidin-5- yl)nicotinate | MS(ES): 442 (M + 1) for C₂₂H₂₁ClFN₅O₂ ¹H NMR (300 MHz, DMSO- d6) δ ppm 1.34 (t, J = 7.06 Hz, 3 H) 1.65-1.89 (m, 4 H) 2.99- 3.26 (m, 4 H) 4.36 (q, J = 7.10 Hz, 2 H) 7.31 (t, J = 9.14 Hz, 1 H) 7.54-7.76 (m, 1 H) 7.94 (s, 1 H) 8.14 (t, J = 2.07 Hz, 1 H) 8.24 (dd, J = 6.88, 2.54 Hz, 1 H) 8.76 (d, J = 2.07 Hz, 1 H) 9.00 (d, J = 1.88 Hz, 1 H) 9.55 (s, 1 H) | 5-bromo-N- (3-chloro-4- fluorophenyl)- 4- (pyrrolidin-1- yl)pyrimidin- 2-amine (Intermediate 104) |
| 148 | 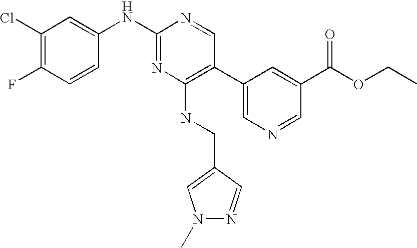 ethyl 5-(2-(3-chloro-4- fluorophenylamino)-4- ((1-methyl-1H-pyrazol- 4- yl)methylamino) pyrimidin-5-yl)- nicotinate | MS(ES): 482 (M + 1) for C₂₃H₂₁ClFN₇O₂ ¹H NMR (300 MHz, DMSO- d6) δ ppm 1.34 (t, J = 7.06 Hz, 3 H) 3.74 (s, 3 H) 4.19-4.52 (m, 4 H) 7.14-7.42 (m, 3 H) 7.54 (s, 1 H) 7.57-7.73 (m, 1 H) 7.87 (s, 1 H) 8.07-8.31 (m, 2 H) 8.78 (d, J = 2.26 Hz, 1H) 9.04 (d, J = 1.88 Hz, 1 H) 9.47 (s, 1 H) | 5-bromo-N²- (3-chloro-4- fluorophenyl)- N⁴-((1- methyl-1H- pyrazol-4- yl)methyl) pyrimidine- 2,4- diamine (Intermediate 106) |
| 149 | 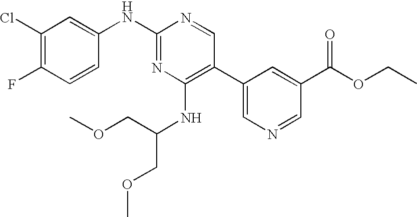 ethyl 5-(2-(3-chloro-4- fluorphenylamino)-4- (1,3-dimethoxypropan- 2-ylamino)pyrimidin-5- yl)nicotinate | MS(ES): 490 (M + 1) for C₂₃H₂₅ClFN₅O₄ ¹H NMR (300 MHz, DMSO- d6) δ ppm 1.23-1.46 (m, 3 H) 3.25 (s, 6 H) 3.35-3.55 (m, 4 H) 4.36 (q, J = 7.10 Hz, 2 H) 4.49- 4.78 (m, 1 H) 6.55 (d, J = 8.48 Hz, 1 H) 7.15-7.41 (m, J = 9.14, 9.14 Hz, 1 H) 7.50-7.71 (m, 1 H) 7.82-7.96 (m, 1 H) 8.16 (dd, J = 6.88, 2.54 Hz, 1 H) 8.23 (t, J = 2.07 Hz, 1 H) 8.78 (d, J = 2.07 Hz, 1 H) 9.04 (d, J = 2.07 Hz, 1 H) 9.49 (s, 1 H) | 5-bromo-N²- (3-chloro-4- fluorophenyl)- N⁴-(1,3- dimethoxy- propan-2- yl)pyrimidine- 2,4-diamine (Intermediate 107) |
| 150 | 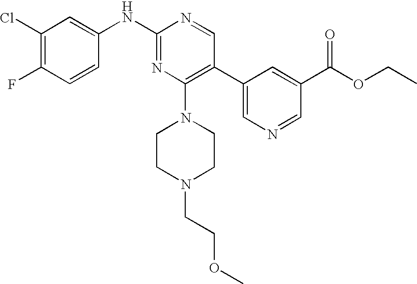 ethyl 5-(2-(3-chloro-4- fluorophenylamino)-4- (4-(2- methoxyethyl)piperazin- 1-yl)pyrimidin-5- yl)nicotinate | MS(ES): 515 (M + 1) for C₂₅H₂₈ClFN₆O₃ ¹H NMR (300 MHz, DMSO- d6) δ ppm 1.35 (t, J = 7.06 Hz, 3 H) 2.29-2.45 (m, 6 H) 3.08- 3.26 (m, 7 H) 3.39 (t, J = 5.75 Hz, 2 H) 4.37 (q, J = 7.03 Hz, 2 H) 7.33 (t, J = 9.14 Hz, 1 H) 7.49- 7.78 (m, 1 H) 8.06-8.28 (m, 2 H) 8.36 (t, J = 2.07 Hz, 1 H) 8.91 (d, J = 2.26 Hz, 1 H) 9.00 (d, J = 1.88 Hz, 1 H) 9.72 (s, 1 H) | 5-bromo-N- (3-chloro-4- fluorophenyl)- 4-[4-(2- methoxyethyl) piperazin-1- yl]pyrimidin- 2-amine (Intermediate 108) |
| 151 | 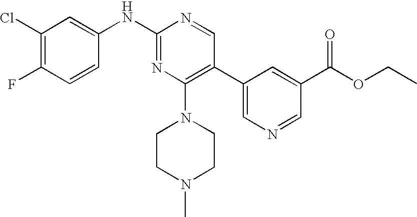 ethyl 5-(2-(3-chloro-4- fluorophenylamino)-4- (4-methylpiperazin-1- yl)pyrimidin-5- yl)nicotinate | MS(ES): 471 (M + 1) for C₂₃H₂₄ClFN₆O₂ ¹H NMR (300 MHz, DMSO- d6) δ ppm 1.35 (t, J = 7.06 Hz, 3 H) 2.14 (s, 3 H) 2.19-2.37 (m, 4 H) 3.12-3.26 (m, 4 H) 4.37 (q, J = 6.97 Hz, 2 H) 7.33 (t, J = 9.14 Hz, 1 H) 7.52-7.73 (m, 1 H) 8.05-8.27 (m, 2 H) 8.29- 8.49 (m, 1 H) 8.92 (d, J = 1.88 Hz, 1 H) 9.00 (d, J = 1.51 Hz, 1 H) 9.73 (s, 1 H) | 5-bromo-N- (3-chloro-4- fluorophenyl)- 4-(4- methylpiperazin- 1- yl)pyrimidin- 2-amine (Intermediate 109) |
| 152 | 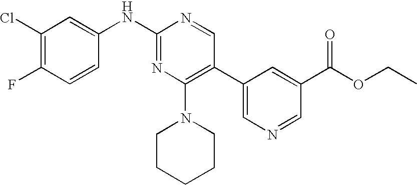 ethyl 5-(2-(3-chloro-4- fluorophenylamino)-4- (piperidin-1- yl)pyrimidin-5- yl)nicotinate | MS(ES): 456 (M + 1) for C₂₃H₂₃ClFN₅O₂ ¹H NMR (300 MHz, DMSO- d6) δ ppm 1.34 (t, J = 6.97 Hz, 3 H) 1.40-1.65 (m, 6 H) 3.10- 3.27 (m, 4 H) 4.36 (q, J = 7.16 Hz, 2 H) 7.33 (t, J = 9.14 Hz, 1 H) 7.50-7.78 (m, 1 H) 8.13 (s, 1 H) 8.22 (dd, J = 6.88, 2.54 Hz, 1 H) 8.35 (t, J = 1.98 Hz, 1 H) 8.91 (d, J = 2.07 Hz, 1 H) 8.99 (d, J = 1.88 Hz, 1 H) 9.68 (s, 1 H) | 5-bromo-N- (3-chloro-4- fluorophenyl)- 4-(piperidin- 1- yl)pyrimidin- 2-amine (Intermediate 100) |
| 153 | 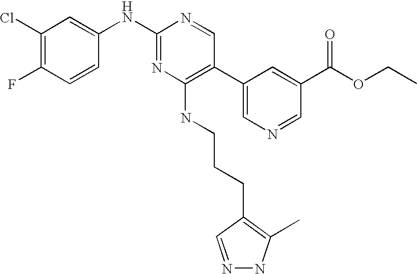 ethyl 5-(2-(3-chloro-4- fluorophenylamino)-4- (3-(5-methyl-1H- pyrazol-4- yl)propylamino) pyrimidin-5-yl) nicotinate | MS(ES): 510 (M + 1) for C₂₅H₂₅ClFN₇O₂ ¹H NMR (300 MHz, DMSO-d₆) δ ppm 1.26-1.42 (m, 3 H) 1.67- 1.89 (m, 2 H) 2.08 (br. s., 3 H) 2.40 (t, J = 7.54 Hz, 2 H) 3.37- 3.48 (m, 2 H) 4.36 (q, J = 7.10 Hz, 2 H) 7.00 (br. s., 1 H) 7.30 (t, J = 9.14 Hz, 1 H) 7.60 (dd, J = 11.77, 4.05 Hz, 1 H) 7.85 (s, 1 H) 8.13-8.38 (m, 2 H) 8.79 (t, J = 1.98 Hz, 1 H) 9.05 (d, J = 1.88 Hz, 1 H) 9.49 (s, 1 H) | 5-bromo-N²- (3-chloro-4- fluorophenyl)- N⁴-(3-(5- methyl-1H- pyrazol-4- yl)propyl) pyrimidine- 2,4- diamine (Intermediate 99) |

### Example 154

### Ethyl 5-(2-[(3-chloro-4-fluorophenyl)amino]-4-{[2-(1H-imidazol-4-yl)ethyl]amino}pyrimidin-5-yl)pyridine-3-carboxylate

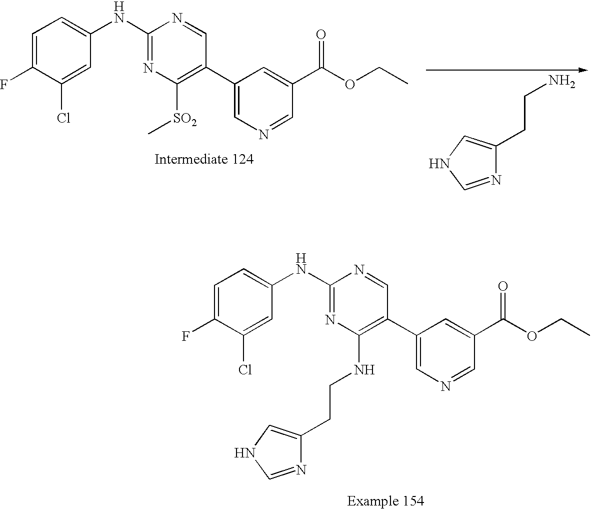

Ethyl 5-{2-[(3-chloro-4-fluorophenyl)amino]-4-(methylsulfonyl)pyrimidin-5-yl}pyridine-3-carboxylate (Intermediate 124, 200 mg, 0.44 mmol) was suspended in NMP (1 mL), treated with N,N-diisopropylethylamine (1 eq) and the 2-(1H-imidazol-4-yl)ethanamine (49 mg, 0.44 mmol). The mixture was heated at 90° C. for 30 min in a sealed tube. The reaction mixture was added to water and stirred for 15 min. The precipitated solid was filtered, washed with water and dried to afford the crude product. It was further purified by flash chromatography (chloroform:methanol (9:1)) to provide the title compound (100 mg).

MS (ES) 482 (M+1) for C₂₃H₂₁ClFN₇O₂.

¹H NMR 300 MHz, DMSO-d₆: δ 1.34 (t, J=6.96 Hz, 3H), 3.16 (br s, 2H), 3.58 (br s, 2H), 4.36 (q, J=7.68 Hz, 2H), 6.82 (s, 1H), 7.09 (br s, 1H), 7.29 (t, J=7.95 Hz, 1H), 7.51 (s, 1H), 7.7 (br s, 1H), 7.86 (s, 1H), 8.16 (br s, 1H), 8.21 (s, 1H), 8.76 (s, 1H), 9.04 (s, 1H), 9.47 (s, 1H), 11.8 (br s, 1H).

The following examples were prepared using the general method described above for Example 154 using ethyl 5-{2-[(3-chloro-4-fluorophenyl)amino]-4-(methylsulfonyl)pyrimidin-5-yl}pyridine-3-carboxylate Intermediate 124, N,N-diisopropylethylamine and the starting material (SM) indicated.

| Ex | Compound | Data | SM |
|---|---|---|---|
| 155 | 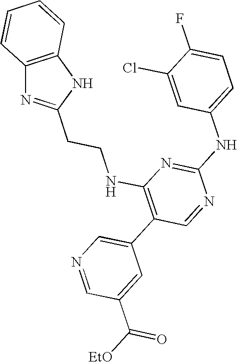 ethyl 5-(4-{[2- (1H-benzimidazol-2- yl)ethyl]amino}- 2-[(3-chloro-4- fluorophenyl)amino]- pyrimidin-5-yl)pyridine-3- carboxylate | MS(ES): 532 (M + 1) for C₂₇H₂₃ClFN₇O₂. ¹H NMR (300 MHz, DMSO-d₆): δ 1.28 (t, J = 7.11 Hz, 3H), 3.12-3.16 (m, 2H), 3.82-3.84 (m, 2H), 4.30 (q, J = 7.20 Hz, 2H), 7.09-7.12 (m, 2H), 7.26 (t, J = 9.00 Hz, 2H), 7.44 (br s, 2H), 7.71 (br s, 1H), 7.89 (s, 1H), 8.16-8.18 (m, 1H), 8.21 (s, 1H), 8.80 (br s, 1H), 9.10 (br s, 1H), 9.52 (br s, 1H), 12.26 (br s, 1H). | 2-(1H- benzimidazol- 2- yl)ethanamine |
| 156 | 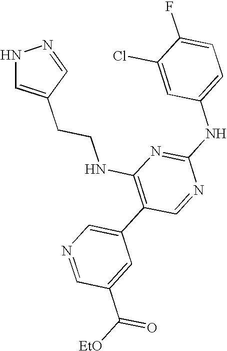 ethyl 5-(2-[(3- chloro-4-fluorophenyl) amino]-4-{[2-(1H- pyrazol-4-yl)ethyl]amino} pyrimidin-5-yl)pyridine-3- carboxylate | MS(ES): 482 (M + 1) for C₂₃H₂₁ClFN₇O₂. ¹H NMR (400 MHz, DMSO-d₆) δ 1.35 (t, J = 7.08 Hz, 3H), 2.73 (t, J = 7.12 Hz, 2H), 4.38 (q, J = 7.12 Hz, 2H), 7.00 (br s, 1H), 7.25 (t, J = 9.08 Hz, 1H), 7.30 (br s, 1H), 7.50 (br s, 1H), 7.64 (m, 1H), 7.85 (s, 1H), 8.18 (dd, J = 2.72, 6.78 Hz, 2H), 8.75 (d, J = 2.04 Hz, 1H), 9.02 (d, J = −13.96 Hz, 1H), 9.47 (s, 1H), 12.56 (br s, 1H). | 2-(1H- pyrazol-4- yl)ethanamine |
| 157 | 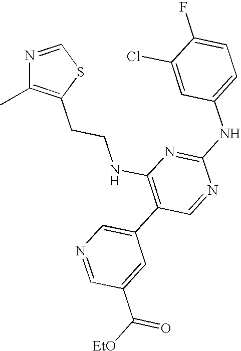 ethyl 5-(2-[(3- chloro-4- fluorophenyl)amino]- 4-{[2-(4-methyl-1,3- thiazol-5- yl)ethyl]amino} pyrimidin-5-yl)pyridine-3- carboxylate | MS(ES): 513 (M + 1) for C₂₄H₂₂ClFN₆O₂S. ¹H NMR (400 MHz, DMSO-d₆): δ 1.34 (t, J = 7.08 Hz, 3H), 2.25 (s, 3H), 3.06 (t, J = 6.92 Hz, 2H), 3.56 (q, J = 6.72 Hz, 2H), 4.38 (q, J = 7.12 Hz, 2H), 7.04 (t, J = 5.80 Hz, 1H), 7.29 (t, J = 9.12 Hz, 1H), 7.65 (ddd, J = 2.72, 4.20, 9.09 Hz, 1H), 7.88 (s, 1H), 8.15 (dd, J = 2.64, 6.90 Hz, 1H), 8.19 (t, J = 2.12 Hz, 1H), 8.75 (d, J = 2.24 Hz, 1H), 8.81 (s, 1H), 9.05 (d, J = 2.00 Hz, 1H), 9.49 (s, 1H). | 2-(4-methyl- 1,3-thiazol-5- yl)ethanamine |

### Example 158

### ethyl (2E)-3-(3-{2-[(3-chloro-4-fluorophenyl)amino]-4-(3,5-dimethyl-1H-pyrazol-1-yl)pyrimidin-5-yl}phenyl)prop-2-enoate

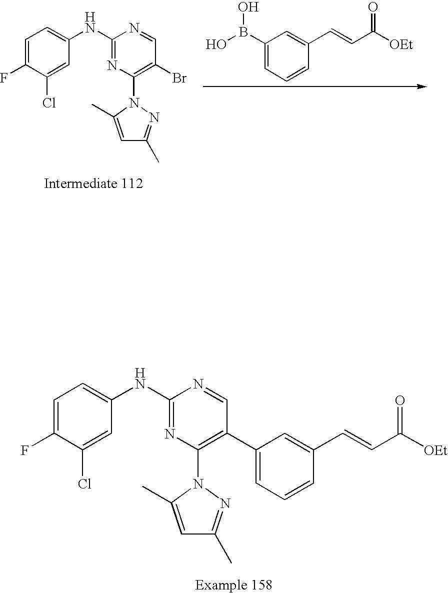

A suspension of 5-bromo-N-(3-chloro-4-fluorophenyl)-4-(3,5-dimethyl-1H-pyrazol-1-yl)pyrimidin-2-amine (Intermediate 112, 1 eq, 3.5 mmol), {3-[(1E)-3-ethoxy-3-oxoprop-1-en-1-yl]phenyl}boronic acid (1.1 eq, 3.95 mmol), [1,1′-bis(diphenylphosphino)ferrocene]dichloropalladium(II) complex with CH₂Cl₂ (10 mol %) and sodium carbonate (1 eq, 3.5 mmol) in acetonitrile/water (20 mL: 5 mL) was degassed and heated to 90° C. for 15-20 min in an oil bath under inert atmosphere. Completion of the reaction was monitored by TLC. The solvent was removed under vacuum and the crude mixture was taken up in CHCl₃ (30 mL). It was then washed with water, brine, dried over sodium sulfate, filtered and concentrated. The crude material was purified by silica gel column chromatography using chloroform-methanol as an eluent to provide the title compound (620 mg, 63%).

MS(ES): 492 (M+1) for C₂₆H₂₃ClFN₅O₂.

¹H NMR 400 MHz, DMSO-d₆: δ 1.25 (dt, J=1.28, 7.06 Hz, 3H), 2.02 (s, 3H), 2.13 (s, 3H), 4.18 (dq, J=1.12, 7.14 Hz, 2H), 6.03 (s, 1H), 6.54 (dd, J=1.24, 16.02 Hz, 1H), 7.01 (d, J=7.24 Hz, 1H), 7.34 (t, J=7.76 Hz, 1H), 7.37-7.42 (m, 2H), 7.56-7.60 (m, 2H), 7.63-7.67 (m, 1H), 8.10 (d, J=6.76 Hz, 1H), 8.87 (d, J=1.36 Hz, 1H), 10.30 (s, 1H).

The following examples were prepared following the general procedure described above for Example 158 using {3-[(1E)-3-ethoxy-3-oxoprop-1-en-1-yl]phenyl}boronic acid, [1,1′-bis(diphenylphosphino)ferrocene]dichloropalladium(II), sodium carbonate and the starting material (SM) listed.

| Ex | Compound | Data | SM |
|---|---|---|---|
| 159 | 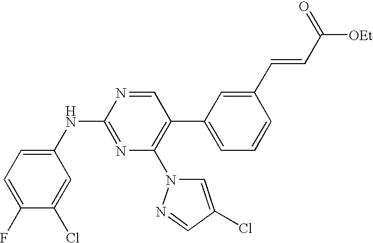 ethyl (2E)-3-(3-{2-[(3- chloro-4- fluorophenyl)amino]-4- (4-chloro-1H-pyrazol-1- yl)pyrimidin-5- yl}phenyl)prop-2-enoate | MS(ES): 498 (M + 1) and 500 (M + 2) for C₂₄H₁₈Cl₂FN₅O₂. ¹H NMR (300 MHz, DMSO- d₆): δ 1.25 (t, J = 7.02 Hz, 3H), 4.18 (q, J = 7.02 Hz, 2H), 6.62 (d, J = 16.05 Hz, 1H), 7.15 (d, J = 7.68 Hz, 1H), 7.34-7.44 (m, 2H), 7.59- 7.64 (m, 3H), 7.71-7.74 (m, 2H), 8.05 (dd, J = 2.19, 6.63 Hz, 1H), 8.47 (s, 1H), 8.71 (s, 1H), 10.30 (s, 1H). | 5-bromo-N- (3-chloro-4- fluorophenyl)- 4-(4-chloro- 1H-pyrazol- 1- yl)pyrimidin- 2-amine (Intermediate 114) |
| 160 | 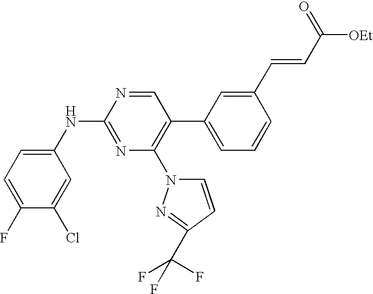 ethyl (2E)-3-(3-{2-[(3-chloro-4- fluorophenyl)amino]-4- [3-(trifluoromethyl)-1H- pyrazol-1-yl]pyrimidin- 5-yl}phenyl)prop-2- enoate | MS(ES): 532 (M + 1) for C₂₅H₁₈ClF₄N₅O₂. ¹H NMR (400 MHz, DMSO- d₆): δ 1.25 (t, J = 7.12 Hz, 3H), 4.18 (q, J = 7.04 Hz, 2H), 6.59 (d, J = 16.0 Hz, 1H), 7.00 (d, J = 2.44 Hz, 1H), 7.13 (d, J = 7.64 Hz, 1H), 7.35-7.43 (m, 2H), 7.62 (s, 2H), 7.68-7.72 (m, 2H), 8.12 (dd, J = 6.68, 2.44 Hz, 1H), 8.35 (s, 1H), 8.83 (s, 1H), 10.44 (s, 1H). | 5-bromo-N- (3-chloro-4- fluorophenyl)- 4-[3- (trifluoro- methyl)-1H- pyrazol-1- yl]pyrimidin- 2-amine (Intermediate 115) |
| 161 | 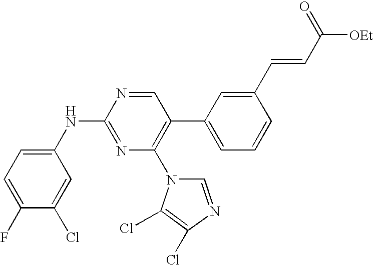 ethyl (2E)-3-(3-{2-[(3-chloro-4- fluorophenyl)amino]-4- (4,5-dichloro-1H- imidazol-1-yl)pyrimidin- 5-yl}phenyl)prop-2- enoate | MS(ES): 532 (M + 1) and 534 (M + 3) for C₂₄H₁₇Cl₃FN₅O₂. ¹H NMR (400 MHz, DMSO- d₆): δ 1.24 (m, 3H), 4.19 (q, J = 7.12 Hz, 2H), 6.63 (d, J = 16.04 Hz, 1H), 7.10 (d, J = 7.76 Hz, 1H), 7.41 (t, J = 7.80 Hz, 2H), 7.61 (d, J = 15.96 Hz, 1H), 7.61 (s, 1H), 7.66 (m, 2H), 8.05 (m, 2H), 8.98 (d, J = 0.88 Hz, 1H), 10.55 (s, 1H). | 5-bromo-N- (3-chloro-4- fluorophenyl)- 4-(4,5- dichloro-1H- imidazol-1- yl)pyrimidin- 2-amine (Intermediate 116) |
| 162 | 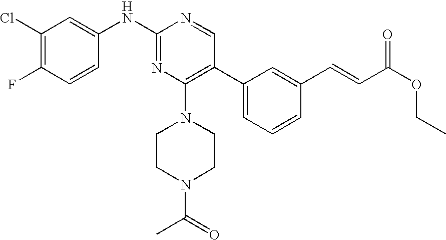 (E)-ethyl 3-(3-(4-(4- acetylpiperazin-1-yl)-2- (3-chloro-4- fluorophenylamino) pyrimidin-5- yl)phenyl)acrylate | MS(ES): 524 (M + 1) for C₂₇H₂₇ClFN₅O₃ ¹H NMR (300 MHz, DMSO- d6) δ ppm 1.25 (t, J = 7.06 Hz, 3H) 1.95 (s, 3H) 3.10-3.57 (m, 8H) 4.19 (q, J = 6.97 Hz, 2H) 6.72 (d, J = 16.01 Hz, 1H) 7.33 (t, J = 9.04 Hz, 1H) 7.42- 7.58 (m, 2H), 7.57-7.77 (m, 3H) 7.81 (s, 1H), 7.99-8.28 (m, 2H), 9.64 (s, 1H) | 1-{4-[5- bromo-2-(3- chloro-4- fluorophenyl amino) pyrimidin-4- yl]piperazin- 1- yl}ethanone (Intermediate 98) |
| 163 | 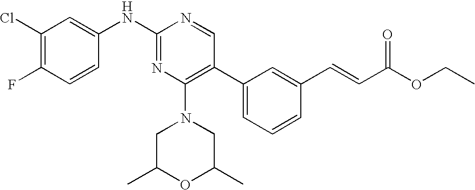 (E)-ethyl 3-(3-(2-(3-chloro-4- fluorophenylamino)-4- (2,6- dimethylmorpholino) pyrimidin-5- yl)phenyl)acrylate | MS(ES): 511 (M + 1) for C₂₇H₂₈ClFN₄O₃ ¹H NMR (300 MHz, DMSO- d6) δ ppm 0.93 (d, J = 6.03 Hz, 6H) 1.25 (t, J = 7.16 Hz, 3H) 2.31-2.45 (m, 2H), 3.42- 3.71 (m, 4H) 4.19 (q, J = 7.16 Hz, 2H) 6.72 (d, J = 16.01 Hz, 1H), 7.32 (t, J = 9.14 Hz, 1H) 7.40-7.62 (m, 3H) 7.61- 7.88 (m, 3H) 8.06 (s, 1H) 8.23 (dd, J = 6.97, 2.45 Hz, 1H) 9.63 (s, 1H) | 5-bromo-N- (3-chloro-4- fluorophenyl)- 4-(2,6- dimethyl- morpholino) pyrimidin-2- amine (Intermediate 95) |
| 164 | 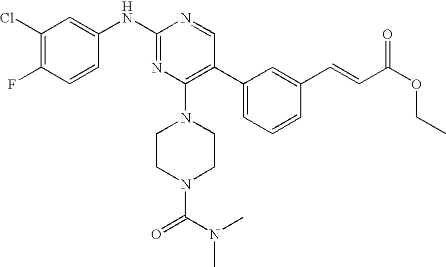 (E)-ethyl 3-(3-(2-(3-chloro-4- fluorophenylamino)-4-(4- (dimethylcarbamoyl) piperazin-1-yl)pyrimidin-5- yl)phenyl)acrylate | MS(ES): 553 (M + 1) for C₂₈H₃₀ClFN₆O₃ ¹H NMR (300 MHz, DMSO- d6) δ ppm 1.25 (t, J = 7.06 Hz, 3H) 2.70 (s, 6H) 2.96-3.15 (m, 4H), 3.17-3.29 (m, 4H) 4.19 (q, J = 6.97 Hz, 2H) 6.72 (d, J = 16.20 Hz, 1H) 7.33 (t, J = 9.04 Hz, 1H) 7.42-7.57 (m, 2H) 7.57-7.75 (m, 3H) 7.80 (s, 1H) 8.08 (s, 1H) 8.14 (dd, J = 6.88, 2.54 Hz, 1H) 9.62 (s, 1H) | 4-(5-bromo- 2-(3-chloro- 4- fluorophenyl amino) pyrimidin-4-yl)- N,N- dimethyl- piperazin-1- carboxamide (Intermediate 101) |
| 165 | 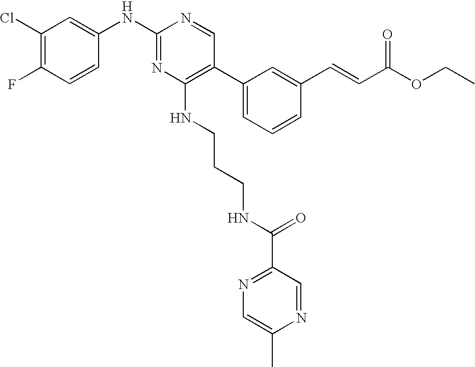 (E)-ethyl 3-(3-(2-(3-chloro-4- fluorophenylamino)-4-(3- (5-methylpiperazine-2- carboxamido)propylamino) pyrimidin-5- yl)phenyl)acrylate | MS(ES): 590 (M + 1) for C₃₀H₂₉ClFN₇O₃ ¹H NMR (300 MHz, DMSO- d6) δ ppm 1.22 (t, J = 7.16 Hz, 3H) 1.69-1.97 (m, 2H) 2.56 (s, 3H) 3.33-3.61 (m, 4H) 4.17 (q, J = 7.10 Hz, 2H) 6.71 (d, J = 16.20 Hz, 1H) 6.83 (t, J = 5.37 Hz, 1H) 7.27 (t, J = 9.14 Hz, 1H) 7.40-7.55 (m, 2H) 7.56-7.63 (m, 1H) 7.66-7.78 (m, 3H) 7.81 (s, 1H) 8.23 (dd, J = 6.78, 2.26 Hz, 1H) 8.55 (d, J = 0.94 Hz, 1H) 8.77-9.11 (m, 2H) 9.40 (s, 1H) | N-(3-(5- bromo-2-(3- chloro-4- fluorophenyl amino) pyrimidin-4- ylamino) propyl)-5- methylpyrazine- 2- carboxamide (Intermediate 102) |
| 166 | 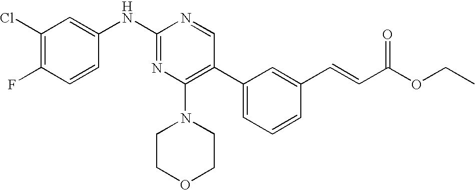 (E)-ethyl 3-(3-(2-(3-chloro-4- fluorophenylamino)-4- morpholinopyrimidin-5- yl)phenyl)acrylate | MS(ES): 483 (M + 1) for C₂₅H₂₄ClFN₄O₃ ¹H NMR (300 MHz, DMSO- D6) δ ppm 1.20 (t, J = 7.16 Hz, 3H) 3.10-3.30 (m, 4H) 3.41- 3.58 (m, 4H) 4.14 (q, J = 7.10 Hz, 2H) 6.66 (d, J = 16.20 Hz, 1H) 7.28 (t, J = 9.04 Hz, 1H) 7.36-7.71 (m, 5H) 7.76 (s, 1H) 7.94-8.17 (m, 2H) 9.64 (s, 1H) | 5-Bromo-N- (3-chloro-4- fluorophenyl)- 4- morpholin-4- ylpyrimidin- 2-amine (Intermediate 111) |
| 167 | 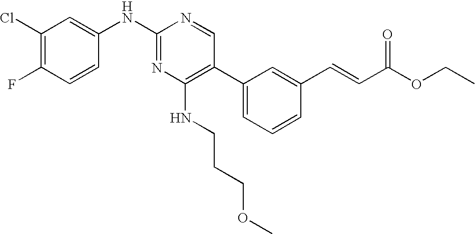 (E)-ethyl 3-(3-(2-(3-chloro-4- fluorophenylamino)-4-(3- methoxypropylamino) pyrimidin-5- yl)phenyl)acrylate | MS(ES): 485 (M + 1) for C₂₅H₂₆ClFN₄O₃ ¹H NMR (300 MHz, DMSO- d6) δ ppm 1.25 (t, J = 7.06 Hz, 3H) 1.60-1.96 (m, 2H) 3.16 (s, 3H) 3.25-3.52 (m, 4H) 4.19 (q, J = 7.16 Hz, 2H) 6.71 (d, J = 16.01 Hz, 1H) 7.31- 7.47 (m, 2H) 7.48-7.61 (m, 2H) 7.63-7.90 (m, 5H) 8.05 (d, J = 4.33 Hz, 1H) 9.97 (s, 1H) | 5-bromo-N²- (3-chloro-4- fluorophenyl)- N⁴-(3- methoxy- propyl) pyrimidine- 2,4-diamine (Intermediate 119) |
| 168 | 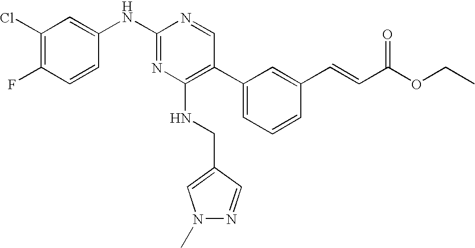 (E)-ethyl 3-(3-(2-(3-chloro-4- fluorophenylamino)-4- ((1-methyl-1H-pyrazol-4- yl)methylamino)pyrimidin- 5-yl)phenyl)acrylate | MS(ES): 507 (M + 1) for C₂₆H₂₄ClFN₆O₂ ¹H NMR (300 MHz, DMSO- d6) δ ppm 1.25 (t, J = 7.06 Hz, 3H) 3.73 (s, 3H) 4.19 (q, J = 7.16 Hz, 2H) 4.40 (d, J = 5.65 Hz, 2H) 6.69 (d, J = 16.01 Hz, 1H) 7.01 (t, J = 5.65 Hz, 1H) 7.17-7.38 (m, 2H) 7.39-7.58 (m, 3H) 7.59-7.78 (m, 4H) 7.82 (s, 1H) 8.18 (dd, J = 6.88, 2.73 Hz, 1H) 9.39 (s, 1H) | 5-bromo-N²- (3-chloro-4- fluorophenyl)- N⁴-((1- methyl-1H- pyrazol-4- yl)methyl) pyrimidine-2,4- diamine (Intermediate 106) |
| 169 | 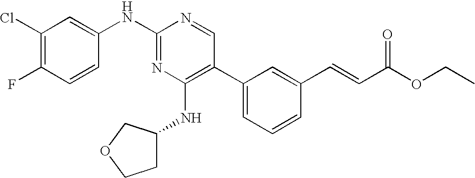 (R,E)-ethyl 3-(3-(2-(3-chloro-4- fluorophenylamino)-4- (tetrahydrofuran-3- ylamino)pyrimidin-5- yl)phenyl)acrylate | MS(ES): 483 (M + 1) for C₂₅H₂₄ClFN₄O₃ ¹H NMR (300 MHz, DMSO- d6) δ ppm 1.25 (t, J = 7.06 Hz, 3H) 1.83-2.04 (m, 1H) 2.08- 2.32 (m, 1H) 3.50-4.02 (m, 4H) 4.19 (q, J = 7.10 Hz, 2H) 4.48-4.78 (m, 1H) 6.55 (d, J = 6.22 Hz, 1H) 6.69 (d, J = 16.01 Hz, 1H) 7.31 (t, J = 9.14 Hz, 1H) 7.37-7.53 (m, 2H) 7.53-7.64 (m, 1H) 7.63-7.79 (m, 3H) 7.85 (s, 1H) 8.25 (dd, J = 6.88, 2.54 Hz, 1H) 9.45 (s, 1H) | (R)-5-bromo- N²-(3-chloro- 4- fluorophenyl)- N⁴- (tetrahydro- furan-3- yl)pyrimidine- 2,4-diamine (Intermediate 103) |
| 170 | 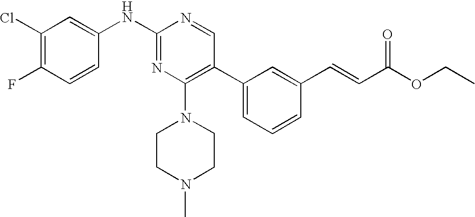 (E)-ethyl 3-(3-(2-(3-chloro-4- fluorophenylamino)-4-(4- methylpiperazin-1- yl)pyrimidin-5- yl)phenyl)acrylate | MS(ES): 496 (M + 1) for C₂₆H₂₇ClFN₅O₂ ¹H NMR (300 MHz, DMSO- d6) δ ppm 1.25 (t, J = 7.06 Hz, 3H) 2.13 (s, 3H) 2.19-2.41 (m, 4H) 3.11-3.29 (m, 4H) 4.19 (q, J = 6.97 Hz, 2H) 6.71 (d, J = 16.01 Hz, 1H) 7.32 (t, J = 9.04 Hz, 1H) 7.41-7.55 (m, 2H) 7.54-7.75 (m, 3H) 7.80 (s, 1H) 8.06 (s, 1H) 8.13- 8.29 (m, 1H) 9.61 (s, 1H) | 5-bromo-N- (3-chloro-4- fluorophenyl)- 4-(4- methyl- piperazin-1- yl)pyrimidin- 2-amine (Intermediate 109) |
| 171 | 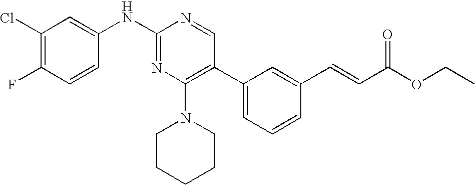 (E)-ethyl 3-(3-(2-(3-chloro-4- fluorophenylamino)-4- (piperidin-1- yl)pyrimidin-5- yl)phenyl)acrylate | MS(ES): 481 (M + 1) for C₂₆H₂₆ClFN₄O₂ ¹H NMR (300 MHz, DMSO- d6) δ ppm 1.25 (t, J = 7.06 Hz, 3H) 1.34-1.63 (m, 6H) 3.15- 3.24 (m, 4H) 4.19 (q, J = 6.97 Hz, 2H) 6.71 (d, J = 16.20 Hz, 1H) 7.31 (t, J = 9.14 Hz, 1H) 7.40-7.52 (m, 2H) 7.54- 7.87 (m, 4H) 8.02 (s, 1H) 8.23 (dd, J = 6.88, 2.54 Hz, 1H) 9.56 (s, 1H) | 5-bromo-N- (3-chloro-4- fluorophenyl)- 4-(piperidin- 1- yl)pyrimidin- 2-amine (Intermediate 100) |
| 172 | 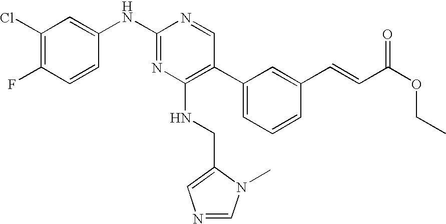 (E)-ethyl 3-(3-(2-(3-chloro-4- fluorophenylamino)-4- ((1-methyl-1H-imidazol-5- yl)methylamino)pyrimidin- 5-yl)phenyl)acrylate | MS(ES): 507 (M + 1) for C₂₆H₂₄ClFN₆O₂ ¹H NMR (300 MHz, DMSO- d6) d ppm 1.25 (t, J = 7.06 Hz, 3H) 3.58 (s, 3H) 4.19 (q, J = 7.10 Hz, 2H) 4.52 (s, 2H) 6.69 (d, J = 16.01 Hz, 1H) 6.79 (s, 1H) 7.03 (t, J = 5.65 Hz, 1H) 7.28 (t, J = 9.14 Hz, 1H) 7.36-7.55 (m, 3H) 7.54- 7.78 (m, 4H) 7.85 (s, 1H) 8.11 (dd, J = 6.78, 2.64 Hz, 1H) 9.39 (s, 1H) | 5-bromo-N²- (3-chloro-4- fluorophenyl)- N⁴-((1- methyl-1H- imidazol-5- yl)methyl) pyrimidine-2,4- diamine (Intermediate 105) |
| 173 | 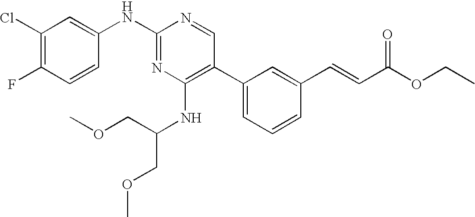 (E)-ethyl 3-(3-(2-(3-chloro-4- fluorophenylamino)-4- (1,3-dimethoxypropan-2- ylamino)pyrimidin-5- yl)phenyl)acrylate | MS(ES): 515 (M + 1) for C₂₆H₂₈ClFN₄O₄ ¹H NMR (300 MHz, DMSO- d6) δ ppm 1.25 (t, J = 7.06 Hz, 3H) 3.25 (s, 6H) 3.37-3.62 (m, 4H) 4.19 (q, J = 6.97 Hz, 2H) 4.38-4.76 (m, 1H) 6.10 (d, J = 8.29 Hz, 1H) 6.68 (d, J = 16.01 Hz, 1H) 7.28 (t, J = 9.14 Hz, 1H) 7.37-7.56 (m, 2H) 7.57-7.81 (m, 4H) 7.88 (s, 1H) 8.15 (dd, J = 6.78, 2.64 Hz, 1H) 9.43 (s, 1H) | 5-bromo-N²- (3-chloro-4- fluorophenyl)- N⁴-(1,3- dimethoxy- propan-2- yl)pyrimidine- 2,4-diamine (Intermediate 107) |
| 174 | 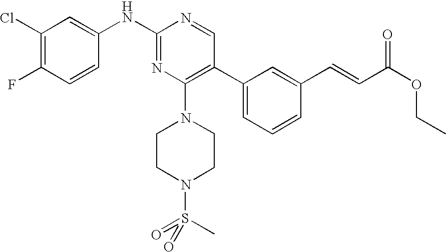 (E)-ethyl 3-(3-(2-(3-chloro-4- fluorophenylamino)-4-(4- (methylsulfonyl)piper- azin-1-yl)pyrimidin-5- yl)phenyl)acrylate | MS(ES): 560 (M + 1) for C₂₆H₂₇ClFN₅O₄S ¹H NMR (300 MHz, DMSO- d6) δ ppm 1.25 (t, J = 7.06 Hz, 3H) 2.86 (s, 3H) 2.99-3.18 (m, 4H) 3.24-3.50 (m, 4H) 4.19 (q, J = 6.97 Hz, 2H) 6.74 (d, J = 16.01 Hz, 1H) 7.36 (t, J = 9.14 Hz, 1H) 7.44-7.58 (m, 2H) 7.57-7.79 (m, 3H) 7.82 (s, 1H) 7.99-8.20 (m, 2H) 9.80 (s, 1H) | 5-Bromo-N- (3-chloro-4- fluorophenyl)- 4-(4- (methyl- sulfonyl) piperazin-1- yl)pyrimidin- 2-amine (Intermediate 130) |

The following examples were prepared using the general method described above for Example 1 using tris(dibenzyledeneacetone)-dipalladium(0), 2-dicyclohexyl phosphino-2′,4′,6′-triiso-propyl-1,1′-biphenyl, sodium carbonate and the starting materials (SM) indicated.

| Ex | Compound | Data | SM |
|---|---|---|---|
| 175 | 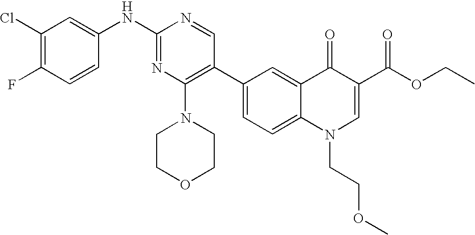 ethyl 6-(2-(3-chloro- 4- fluorophenylamino)- 4- morpholinopyrimidin- 5-yl)-1-(2- methoxyethyl)-4-oxo- 1,4-dihydroquinoline- 3-carboxylate | MS(ES): 582 (M + 1) for C₂₉H₂₉ClFN₅O₅ ¹H NMR (300 MHz, DMSO-d6) δ ppm 1.28 (t, J = 7.06 Hz, 3 H) 3.08-3.28 (m, 7 H) 3.47-3.64 (m, 4 H) 3.69 (t, J = 4.33 Hz, 2 H) 4.23 (q, J = 7.10 Hz, 2 H) 4.59 (t, J = 4.05 Hz, 2 H) 7.33 (t, J = 9.14 Hz, 1 H) 7.55-7.74 (m, 1 H) 7.81- 8.02 (m, 2 H) 8.05-8.26 (m, 2 H) 8.34 (d, J = 1.32 Hz, 1 H) 8.58 (s, 1 H) 9.68 (s, 1 H) | Ethyl 1-(2- methoxyethyl)- 4-oxo-6- (4,4,5,5- tetramethyl- 1,3,2- dioxaborolan- 2-yl)-1,4- dihydroquinoline- 3- carboxylate (Intermediate 134) and 5-Bromo-N- (3-chloro-4- fluorophenyl- 4- morpholin-4- ylpyrimidin- 2-amine (Intermediate 111) |
| 176 | 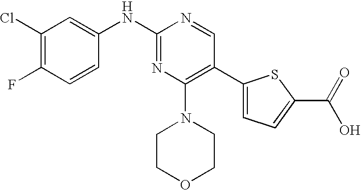 5-(2-(3-chloro-4- fluorophenylamino)- 4- morpholinopyrimidin- 5-yl)thiophene-2- carboxylic acid | MS(ES): 435 (M + 1) for C₁₉H₁₆ClFN₄O₃S ¹H NMR (300 MHz, DMSO-d6) δ ppm 3.14-3.37 (m, 4 H) 3.75- 3.98 (m, 4 H) 7.27 (d, J = 3.77 Hz, 1 H) 7.34 (t, J = 9.14 Hz, 1 H) 7.48- 7.65 (m, 1 H) 7.69 (d, J = 3.77 Hz, 1 H) 8.10 (dd, J = 6.88, 2.54 Hz, 1 H) 8.21 (s, 1 H) 9.82 (s, 1 H) 13.10 (s, 1 H) | 2- carboxythio- phene-5- boronic acid and 5-Bromo-N- (3-chloro-4- fluorophenyl)- 4- morpholin-4- ylpyrimidin- 2-amine (Intermediate 111) |
| 177 | 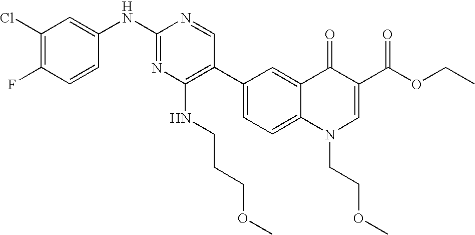 ethyl 6-(2-(3-chloro- 4- fluorophenylamino)- 4-(3- methoxypropylamino)- pyrimidin-5-yl)-1- (2-methoxyethyl)-4- oxo-1,4- dihydroquinoline-3- carboxylate | MS(ES): 584 (M + 1) for C₂₉H₃₁ClFN₅O₅ ¹H NMR (300 MHz, DMSO-d6) δ ppm 1.28 (t, J = 7.06 Hz, 3 H) 1.65-1.94 (m, 2 H) 3.17 (s, 3 H) 3.24 (s, 3 H) 3.34-3.53 (m, 4 H) 3.57-3.83 (m, 2 H) 4.23 (q, J = 7.16 Hz, 2 H) 4.60 (t, J = 4.71 Hz, 2 H) 6.77 (t, J = 5.18 Hz, 1 H) 7.29 (t, J = 9.14 Hz, 1 H) 7.55- 7.71 (m, 1 H) 7.70-7.80 (m, 1 H) 7.82 (s, 1 H) 7.93 (d, J = 8.85 Hz, 1 H) 8.14-8.33 (m, 2 H) 8.59 (s, 1 H) 9.42 (s, 1 H) | Ethyl 1-(2- methoxyethyl)- 4-oxo-6- (4,4,5,5- tetramethyl- 1,3,2- dioxaborolan- 2-yl)-1,4- dihydroquinoline- 3- carboxylate (Intermediate 134) and 5-bromo-N²- (3-chloro-4- fluorophenyl)- N⁴-(3- methoxy- propyl)- pyrimidine- 2,4-diamine (Intermediate 119) |
| 178 | 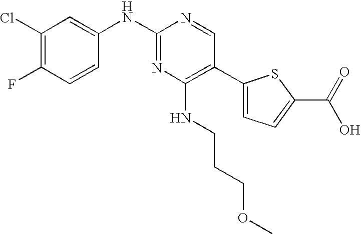 5-(2-(3-chloro-4- fluorophenylamino)- 4-(3- methoxypropylamino)- pyrimidin-5- yl)thiophene-2- carboxylic acid | MS(ES): 437 (M + 1) for C₁₉H₁₈ClFN₄O₃S ¹H NMR (300 MHz, DMSO-d6) δ ppm 1.71-1.94 (m, 2 H) 3.20 (s, 3 H) 3.32-3.58 (m, 4 H) 6.97 (t, J = 5.09 Hz, 1 H) 7.20 (d, J = 3.77 Hz, 1 H) 7.29 (t, J = 9.04 Hz, 1 H) 7.50-7.69 (m, 1 H) 7.73 (d, J = 3.96 Hz, 1 H) 7.97 (s, 1 H) 8.19 (dd, J = 6.97, 2.64 Hz, 1 H) 9.56 (s, 1 H) 13.09 (s, 1 H) | 2- carboxythio- phene-5- boronic acid and 5-bromo-N²- (3-chloro-4- fluorophenyl)- N⁴-(3- methoxy- propyl)- pyrimidine- 2,4-diamine (Intermediate 119) |
| 179 | 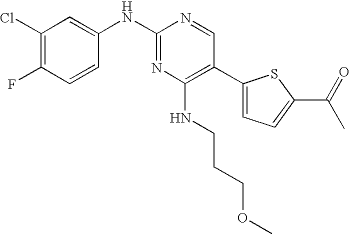 1-(5-(2-(3-chloro-4- fluorophenylamino)- 4-(3- methoxypropylamino)- pyrimidin-5- yl)thiophen-2- yl)ethanone | MS(ES): 435 (M + 1) for C₂₀H₂₀ClFN₄O₂S ¹H NMR (300 MHz, DMSO-d6) δ ppm 1.69-1.95 (m, 2 H) 2.55 (s, 3 H) 3.20 (s, 3 H) 3.29-3.58 (m, 4 H) 7.17-7.44 (m, 2 H) 7.45- 7.70 (m, 2 H) 7.91-8.04 (m, 2 H) 8.09 (dd, J = 6.88, 2.35 Hz, 1H) 9.95 (s, 1 H) | 5-acetyl-2- thiophene- boronic acid and 5-bromo-N²- (3-chloro-4- fluorophenyl)- N⁴-(3- methoxy- propyl)- pyrimidine- 2,4-diamine (Intermediate 119) |
| 180 | 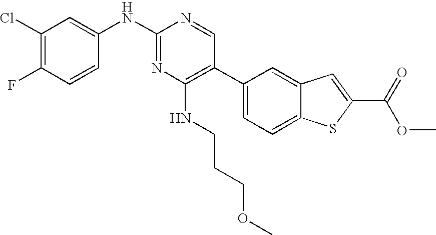 methyl 5-(2-(3- chloro-4- fluorophenylamino)- 4-(3- methoxypropylamino)- pyrimidin-5- yl)benzo[b]thiopene- 2-carboxylate | MS(ES): 501 (M + 1) for C₂₄H₂₂ClFN₄O₃S ¹H NMR (300 MHz, DMSO-d6) δ ppm 1.63-1.94 (m, 2 H) 3.15 (s, 3 H) 3.34-3.52 (m, 4 H) 3.90 (s, 3 H) 6.73 (t, J = 5.27 Hz, 1 H) 7.28 (t, J = 9.14 Hz, 1 H) 7.51 (dd, J = 8.48, 1.70 Hz, 1 H) 7.57-7.72 (m, 1 H) 7.82 (s, 1 H) 8.01 (s, 1 H) 8.15 (d, J = 8.48 Hz, 1 H) 8.20- 8.35 (m, 2 H) 9.40 (s, 1 H) | methyl 5- (4,4,5,5- tetramethyl- 1,3,2- dioxaborolan- 2-yl)-1- benzothio- phene-2- carboxylate and 5-bromo-N²- (3-chloro-4- fluorophenyl)- N⁴-(3- methoxy- propyl)- pyrimidine- 2,4-diamine (Intermediate 119) |
| 181 | 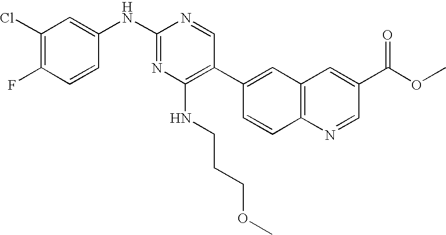 methyl 6-(2-(3- chloro-4- fluorophenylamino)- 4-(3- methoxypropylamino)- pyrimidin-5- yl)quinoline-3- carboxylate | MS(ES): 496 (M + 1) for C₂₅H₂₃ClFN₅O₃ ¹H NMR (300 MHz, DMSO-d₆) δ ppm 1.80 (quin, J = 6.45 Hz, 2 H) 3.16 (s, 3 H) 3.24-3.53 (m, 4 H) 3.98 (s, 3 H) 7.29-7.66 (m, 2 H) 7.83-8.09 (m, 3 H) 8.09- 8.36 (m, 3 H) 9.09 (d, J = 1.51 Hz, 1 H) 9.38 (d, J = 2.07 Hz, 1 H) 10.38 (br. s., 1 H) | Methyl 6- (4,4,5,5- tetramethyl- 1,3,2- dioxaborolan- 2- yl)quinoline- 3-carboxylate (Intermediate 135) and 5-bromo-N²- (3-chloro-4- fluorophenyl)- N⁴-(3- methoxy- propyl)- pyrimidine- 2,4-diamine (Intermediate 119) |
| 182 | 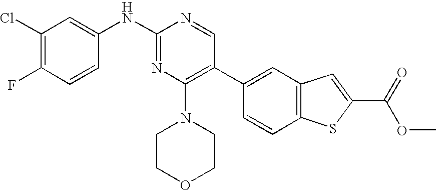 methyl 5-(2-(3- chloro-4- fluorophenylamino)- 4- morpholinopyrimidin- 5- yl)benzo[b]thiophene- 2-carboxylate | MS(ES): 499 (M + 1) for C₂₄H₂₀ClFN₄O₃S ¹H NMR (300 MHz, DMSO-d₆) δ ppm 3.14-3.29 (m, 4 H) 3.43- 3.67 (m, 4 H) 3.91 (s, 3 H) 7.33 (t, J = 9.14 Hz, 1 H) 7.55-7.77 (m, 2 H) 8.03-8.21 (m, 4 H) 8.26 (s, 1 H) 9.65 (s, 1 H) | methyl 5- (4,4,5,5- tetramethyl- 1,3,2- diaoxaborolan- 2-yl)-1- benzothio- phene-2- carboxylate and 5-Bromo-N- (3-chloro-4- fluorophenyl)- 4- morpholin-4- ylpyrimidin- 2-amine (Intermediate 111) |

### Example 183

### Ethyl (2E)-3-[3-(2-[(3-chloro-4-fluorophenyl)amino]-4-{[2-(1H-imidazol-4-yl)ethyl]amino}pyrimidin-5-yl)phenyl]prop-2-enoate

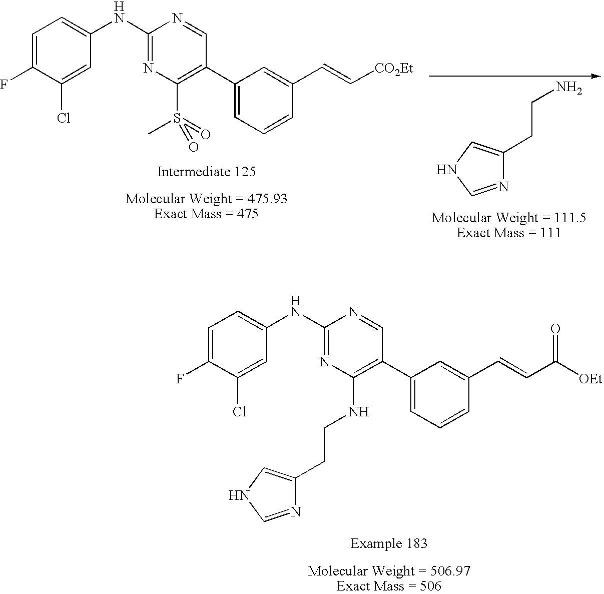

A solution of 2-(1H-imidazol-4-yl)ethanamine (46 mg, 0.4 mmol) in THF (1 mL) was added slowly by syringe to a stirred suspension of sodium hydride (60%, 16 mg, 0.4 mmol) in THF (1 ml) at 0° C. After 30 min, ethyl (2E)-3-(3-{2-[(3-chloro-4-fluorophenyl)amino]-4-(methylsulfonyl)pyrimidin-5-yl}phenyl)prop-2-enoate (Intermediate 125, 200 mg, 0.4 mmol) in THF (2 mL) was added slowly by syringe to the stirred mixture while maintaining the temperature at 0° C. The mixture was stirred under nitrogen for 2 h and poured into ice-water, extracted with ethyl acetate (3×50 mL). The ethyl acetate layer was then washed with brine, dried over Na₂SO₄, filtered and concentrated. The crude product was purified by column chromatography using 1% MeOH in CHCl₃ to yield the title compound (150 mg).

MS(ES): 506 (M+1) for C₂₆H₂₄ClFN₆O₂.

¹H NMR 400 MHz, DMSO-d₆: δ 1.26 (t, J=7.08 Hz, 3H), 2.81-2.85 (m, 2H), 3.62-3.94 (m, 2H), 4.20 (q, J=7.12 Hz, 2H), 6.68 (d, J=16.08 Hz, 1H), 6.84 (br s, 1H), 6.93 (s, 1H), 7.26 (t, J=9.12 Hz, 1H), 7.38 (d, J=7.92 Hz, 1H), 7.48 (t, J=7.64 Hz, 1H), 7.66-7.76 (m, 4H), 7.84 (s, 1H), 8.17 (dd, J=2.64, 6.82 Hz, 1H), 9.41 (s, 1H), 12.50 (br s, 1H).

The following examples were prepared using the general method described above for Example 183 using ethyl (2E)-3-(3-{2-[(3-chloro-4-fluorophenyl)amino]-4-(methylsulfonyl)pyrimidin-5-yl}phenyl)prop-2-enoate Intermediate 125, sodium hydride and the starting material (SM) indicated.

| Ex | Compound | Data | SM |
|---|---|---|---|
| 184 | 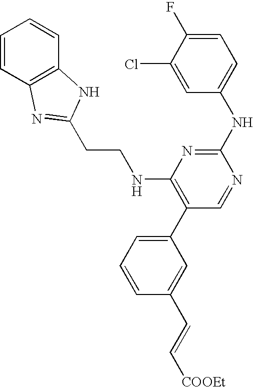 ethyl (2E)-3-[3-(4-{[2- (1H-benzimidazol-2- yl)ethyl]amino}-2-[(3- chloro-4- fluorophenyl)amino]- pyrimidin-5-yl)phenyl]- prop-2-enoate | MS(ES): 557 (M + 1) for C₃₀H₂₆ClFN₆O₂ ¹H NMR (300 MHz, DMSO- d₆): δ 1.69 (t, J = Hz, 3H), 3.15-3.17 (m, 2H), 3.86-3.88 (m, 2H), 4.18 (q, J = 6.99 Hz, 2H), 6.67 (d, J = 15.81 Hz, 1H), 6.99 (br s, 1H), 7.11 (dd, J = 3.06, 5.81 Hz, 1H), 7.25 (t, J = 9.00 Hz, 1H), 7.38-7.43 (m, 4H) 7.60-7.70 (m, 4H), 7.85 (s, 1H), 8.18 (d, J = 4.83 Hz, 1H), 9.44 (s, 1H), 12.27 (br s, 1H). | 2-(1H- benzimidazol- 2- yl)ethanamine |
| 185 | 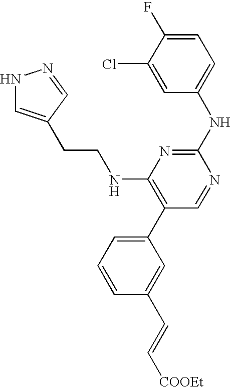 ethyl (2E)-3-[3-(2-[(3- chloro-4- fluorophenyl)amino]-4- {[2-(1H-pyrazol-4- yl)ethyl]amino}-pyrimidin- 5-yl)phenyl]prop-2- enoate | MS(ES): 507 (M + 1) for C₂₆H₂₄ClFN₆O₂. ¹H NMR (400 MHz, DMSO- d₆): δ 1.30-1.34 (m, 3H), 2.82 (m, 2H), 3.66 (m, 2H), 4.25 (m, 2H), 6.55 (d, J = 16.04 Hz, 1H), 7.10 (m, 1H), 7.30 (m, 1H), 7.4-7.6 (m, 4H), 7.60 (m, 1H), 7.62-7.80 (m, 2H), 8.0-8.1 (m, 1H). | 2-(1H- pyrazol-4- yl)ethanamine |
| 186 | 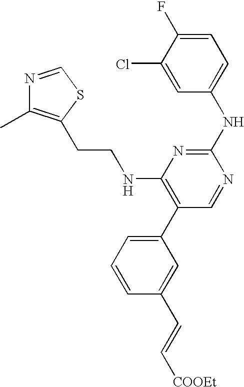 ethyl (2E)-3-[3-(2-[(3- chloro-4- fluorophenyl)amino]-4- {[2-(4-methyl-1,3- thiazol-5- yl)ethyl]amino}pyrimidin- 5-yl)phenyl]prop-2- enoate | MS(ES): 538 (M + 1) for C₂₇H₂₅ClFN₅O₂S. ¹H NMR (400 MHz, DMSO- d₆): δ 1.26 (t, J = 7.08 Hz, 3H), 2.23 (s, 3H), 3.07 (t, J = 6.64 Hz, 2H), 3.58-3.59 (m, 2H), 4.19 (q, J = 7.16 Hz, 2H), 6.68 (d, J = 16.04 Hz, 1H), 6.80 (br s, 1H), 7.28 (t, J = 9.08 Hz, 1H), 7.39 (d, J = 7.48 Hz, 1H), 7.49 (t, J = 7.76 Hz, 1H), 7.64 (br s, 2H), 7.69- 7.72 (m, 2H), 7.84 (d, J = 1.52 Hz, 1H), 8.15 (dd, J = 2.48, 6.72 Hz, 1H), 8.80 (s, 1H), 9.41 (s, 1H). | 2-(4-methyl- 1,3-thiazol-5- yl)ethanamine |
| 187 | 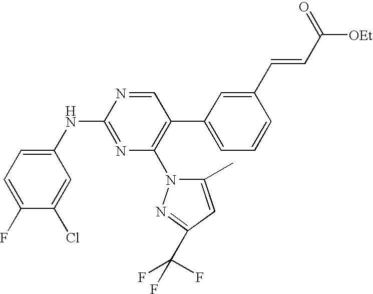 ethyl (2E)-3-(3-{2-[3- chloro-4- fluorophenyl)amino]-4- [5-methyl-3- (trifluoromethyl)-1H- pyrazol-1-yl]pyrimidin- 5-yl}phenyl)prop-2- enoate | MS(ES 546 (M + 1) for C₂₆H₂₀ClF₄N₅O₂. ¹H NMR (400 MHz, DMSO- d₆): δ 1.25 (t, J = 7.08 Hz, 3H), 2.20 (s, 3H), 4.18 (q, J = 7.12 Hz, 2H), 6.55 (d, J = 16.04 Hz, 1H), 6.72 (s, 1H), 7.03 (d, J = 7.76 Hz, 1H), 7.35 (t, J = 7.76 Hz, 1H), 7.40 (br s, 1H), 7.41 (t, J = 9.12 Hz, 1H), 7.55 (d, J = 16.04 Hz, 1H), 7.63-7.67 (m, 2H), 8.08 (dd, J = 2.08, 6.64 Hz, 1H), 8.98 (s, 1H), 10.47 (br s, 1H). | (5-Methyl-3- trifluoro- methyl-1H- pyrazole) |
| 188 | 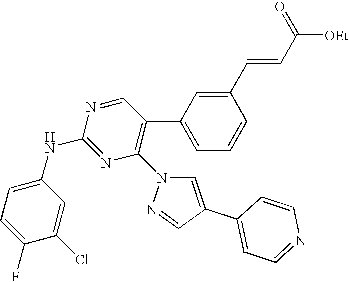 ethyl (2E)-3-(3-{2-[(3- chloro-4- fluorophenyl)amino]-4- [4-(pyridin-4-yl)-1H- pyrazol-1-yl]pyrimidin- 5-yl}phenyl)prop-2- enoate | MS(ES): 541.2 (M + 1) for C₂₉H₂₂ClFN₆O₂. ¹H NMR (400 MHz, DMSO- d₆): δ 1.24 (t, J = 7.20 Hz, 3H), 4.17 (q, J = 7.20 Hz, 2H), 6.63 (d, J = 16.00 Hz, 1H), 7.19 (d, J = 6.00 Hz, 1H), 7.37 (t, J = 8.40 Hz, 1H), 7.44 (t, J = 9.20 Hz, 1H), 7.64 (d, J = 16.00 Hz, 1H), 7.61- 7.85 (m, 4H), 8.16 (dd, J = 2.80, 6.80 Hz, 1H), 8.27 (s, 1H), 8.58 (d, J = 6.00 Hz, 1H), 8.76 (s, 1H), 9.00 (s, 1H), 10.36 (s, 1H). | 4-(1H- Pyrazol-4- yl)-pyridine |
| 189 | 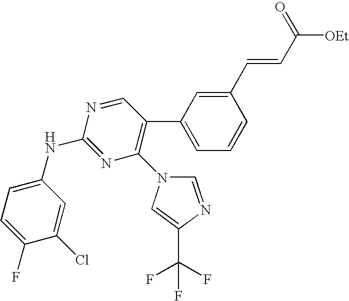 ethyl (2E)-3-(3-{2-[(3- chloro-4- fluorophenyl)amino]-4- [4-(trifluoromethyl)-1H- imidazol-1-yl]pyrimidin- 5-yl}phenyl)prop-2-enoate | MS(ES): 532.2 (M + 1) for C₂₅H₁₈ClF₄N₅O₂. ¹H NMR (400 MHz, DMSO- d₆): δ 1.26 (t, J = 7.20 Hz, 3H), 4.20 (q, J = 7.20 Hz, 2H), 6.64 (d, J = 16.00 Hz, 1H), 7.27 (d, J = 7.60 Hz, 1H), 7.41-7.49 (m, 2H), 7.64 (d, J = 16.00 Hz, 1H), 7.69- 7.73 (m, 2H), 7.76 (d, J = 7.60 Hz, 1H), 7.82 (s, 1H), 7.95 (s, 2H), 8.08 (dd, J = 2.40, 6.80 Hz, 1H), 8.83 (s, 1H). | 4- Trifluoro- methyl-1H- imidazole |
| 190 | 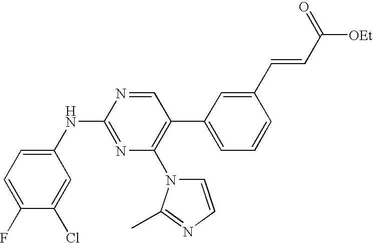 ethyl (2E)-3-(3-{2-[(3- chloro-4- fluorophenyl)amino]-4- (2-methyl-1H-imidazol- 1-yl)pyrimidin-5- yl}phenyl)prop-2-enoate | MS(ES): 478 (M + 1) for C₂₅H₂₁ClFN₅O₂. ¹H NMR (300 MHz, DMSO- d₆): δ 1.25 (t, J = 7.11 Hz, 3H), 2.1 (s, 3H), 4.18 (q, J = 7.11 Hz, 2H), 6.59 (d, J = 16.02 Hz, 1H), 6.82 (s, 1H), 7.08 (br s, 2H), 7.35-7.43 (m, 2H), 7.52-7.55 (m, 2H), 7.65- 7.68 (m, 2H), 8.17 (d, J = 4.89 Hz, 1H), 8.87 (s, 1H), 10.36 (s, 1H). | 2-Methyl- 1H-imidazole |
| 191 | 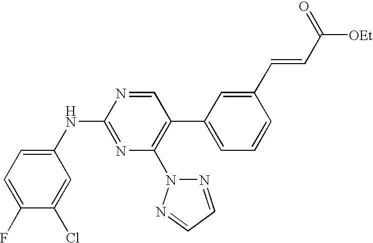 ethyl (2E)-3-(3-{2-[(3- chloro-4- fluorophenyl)amino]-4- (2H-1,2,3-triazol-2- yl)pyrimidin-5- yl}phenyl)prop-2-enoate | MS(ES): 465 (M + 1) for C₂₃H₁₈ClFN₆O₂. ¹H NMR (400 MHz, DMSO- d₆): δ 1.25 (t, J = 7.12 Hz, 3H), 4.18 (q, J = 7.12 Hz, 2H), 6.62 (d, J = 16.04 Hz, 1H), 6.99 (d, J = 7.72 Hz, 1H), 7.33 (t, J = 7.68 Hz, 1H), 7.40 (t, J = 9.08 Hz, 1H), 7.56 (s, 1H), 7.59 (d, J = 16.40 Hz, 1H), 7.65 (d, J = 7.76 Hz, 1H), 7.72-7.75 (m, 1H), 8.09 (s, 2H), 8.19 (dd, J = 2.36, 6.70 Hz, 1H), 8.87 (s, 1H), 10.50 (s, 1H). | 1H-[1,2,3]Triazole |
| 192 | 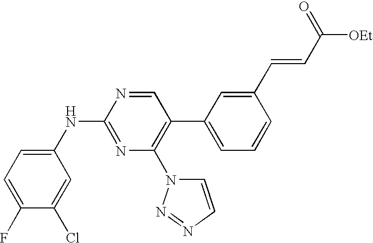 ethyl (2E)-3-(3-{2-[(3- chloro-4- fluorophenyl)amino]-4- (1H-1,2,3-triazol-1- yl)pyrimidin-5- yl}phenyl)prop-2-enoate | MS(ES): 465 (M + 1) for C₂₃H₁₈ClFN₆O₂. ¹H NMR (400 MHz, DMSO- d₆): δ 1.26 (t, J = 6.40 Hz, 3H), 4.19 (q, J = 7.20 Hz, 2H), 6.62 (d, J = 16.00 Hz, 1H), 7.10 (d, J = 7.20 Hz, 1H), 7.37 (t, J = 7.60 Hz, 1H), 7.42 (t, J = 9.60 Hz, 1H), 7.60 (d, J = 16.00 Hz, 1H), 7.61 (s, 1H), 7.68 (d, J = 7.60 Hz, 1H), 7.72-7.74 (m, 1H), 7.93 (s, 1H), 8.08 (d, J = 5.60 Hz, 1H), 8.52 (s, 1H), 8.91 (s, 1H), 10.50 (s, 1H). | 1H- [1,2,3]Triazole |
| 193 | 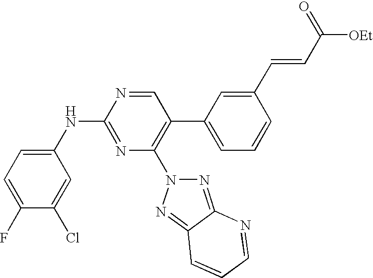 ethyl (2E)-3-(3-{2-[(3- chloro-4- fluorophenyl)amino]-4- (2H-[1,2,3]triazolo[4,5- b]pyridin-2-yl)pyrimidin- 5-yl}phenyl)prop-2- enoate | MS(ES): 518 (M + 1) for C₂₆H₂₁ClFN₇O₂. ¹H NMR (300 MHz, DMSO- d₆): δ 1.24 (t, J = 7.20 Hz, 3H), 4.16 (q, J = 7.20 Hz, 2H), 6.50 (d, J = 16.02 Hz, 1H), 6.90 (d, 1H), 7.25 (t, 1H), 7.41 (t, 1H), 7.56-7.63 (m, 3H), 7.70-7.80 (m, 1H), 8.15 (dd, 1H), 8.51 (dd, J = 1.47, 8.71 Hz, 1H), 8.90 (dd, J = 1.41, 4.03 Hz, 1H), 9.04 (s, 1H), 10.66 (s, 1H). | 1H- [1,2,3]Triazolo- [4,5-b]pyridine |
| 194 | 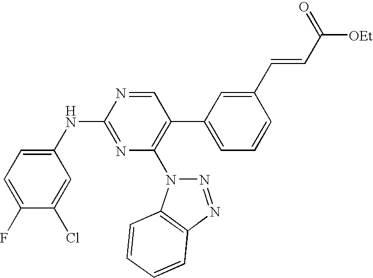 ethyl (2E)-3-(3-{4-(1H- benzotriazol-1-yl)-2-[(3- chloro-4- fluorophenyl)amino]- pyrimidin-5-yl}phenyl)prop- 2-enoate | MS(ES): 515.2 (M + 1) for C₂₇H₂₀ClFN₆O₂. ¹H NMR (400 MHz, DMSO- d₆): δ 1.25 (t, J = 7.28 Hz, 3H), 4.17 (q, J = 7.08 Hz, 2H), 6.58 (d, J = 16.04 Hz, 1H), 7.10 (d, J = 7.68 Hz, 1H), 7.30 (t, J = 7.44 Hz, 1H), 7.38-7.42 (m, 2H), 7.49-7.69 (m, 5H), 7.89-7.95 (m, 1H), 8.13-8.17 (m, 2H), 8.92 (s, 1H), 10.44 (s, 1H). | 1H- Benzotriazole |
| 195 | 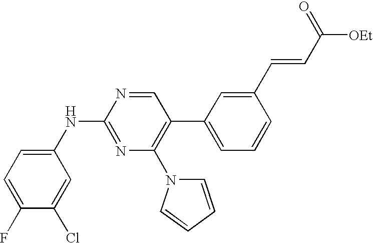 ethyl (2E)-3-(3-{2-[(3- chloro-4- fluorophenyl)amino]-4- (1H-pyrrol-1- yl)pyrimidin-5- yl}phenyl)prop-2-enoate | MS(ES): 463 (M + 1) for C₂₅H₂₀ClFN₄O₂. ¹H NMR (300 MHz, DMSO- d₆): δ 1.24 (t, J = 7.05 Hz, 3H), 4.18 (q, J = 7.02 Hz, 2H), 6.16 (br s, 2H), 6.65 (d, J = 16.05 Hz, 1H),6.91 (br s, 2H), 7.08 (br s, 2H), 7.27 (d, J = 7.62 Hz, 1H), 7.47-7.37 (m, 2H), 7.74-7.80 (m, 2H), 8.11 (d, J = 4.29 Hz, 1H), 8.58 (s, 1H), 10.16 (s, 1H). | 1H-Pyrrole |

The following examples were prepared using the general method described above for Example 1 using {3-[(1E)-3-ethoxy-3-oxoprop-1-en-1-yl]phenyl}boronic acid, tris(dibenzylideneacetone)dipalladium(0), 2-dicyclohexylphosphino-2′,4′,6′-triisopropyl-1,1′-biphenyl, sodium carbonate and the starting material (SM) indicated.

| Ex | Compound | Data | SM |
|---|---|---|---|
| 196 | 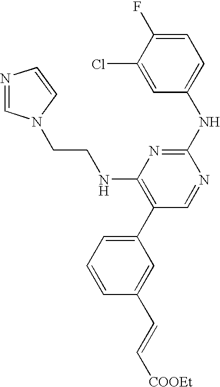 ethyl (2E)-3-[3-(2- [(3-chloro-4- fluorophenyl)amino]- 4-{[2-(1H- imidazol-1- yl)ethyl]amino}- pyrimidin-5- yl)phenyl]prop-2- enoate | MS(ES): 507 (M + 1) for C₂₆H₂₄ClFN₆O₂. ¹H NMR (400 MHz, DMSO-d₆): δ 1.27 (t, J = 7.08 Hz, 3H), 3.68 (q, J = 5.96 Hz, 2H), 4.18-4.24 (m, 4H), 6.68 (d, J = 16.04 Hz, 1H), 6.72 (t, J = 5.76 Hz, 1H), 6.89 (s, 1H), 7.15 (s, 1H), 7.28-7.35 (m, 2H), 7.47 (t, J = 7.68 Hz, 1H), 7.58-7.64 (m, 3H), 7.67 (d, J = 16.16 Hz, 1H), 7.71 (d, J = 7.68 Hz, 1H), 7.84 (s, 1H), 8.16 (dd, J = 2.48, 6.82 Hz, 1H), 9.40 (s, 1H). | 5-bromo-N²- (3-chloro-4- fluorophenyl)- N⁴-[2-(1H- imidazol-1- yl)ethyl]- pyrimidin-2,4- diamine (Intermediate 64) |
| 197 | 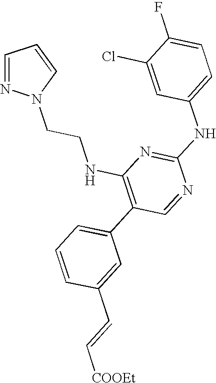 ethyl (2E)-3-[3-(2- [(3-chloro-4- fluorophenyl)amino]- 4-{[2-(1H- pyrazol-1- yl)ethyl]amino}- pyrimidin-5- yl)phenyl]prop-2- enoate | MS(ES): 507 (M + 1) for C₂₆H₂₄ClFN₆O₂. ¹H NMR (400 MHz, DMSO-d₆): δ 1.27 (t, J = 7.08 Hz,3H), 3.76 (q, J = 5.72 Hz, 2H), 4.21 (q, J = 7.16 Hz, 2H), 4.38 (t, J = 6.00 Hz, 2H), 6.26 (t, J = 1.88 Hz, 1H), 6.66 (d, J = 16.04 Hz, 1H), 6.72 (t, J = 5.36 Hz, 1H), 7.30 (t, J = 9.12 Hz, 1H), 7.36 (d, J = 7.68 Hz, 1H), 7.45 (s, 1H), 7.49 (d, J = 7.68 Hz, 1H), 7.60 (s, 1H), 7.67 (d, J = 16.12 Hz, 1H), 7.69-7.72 (m, 3H), 7.85 (s, 1H), 8.15 (dd, J = 2.56, 6.88 Hz, 1H), 9.44 (s, 1H). | 5-bromo-N²- (3-chloro-4- fluorophenyl)- N⁴-[2-(1H- pyrazol-1- yl)ethyl]- pyrimidine-2,4- diamine (Intermediate 91) |
| 198 | 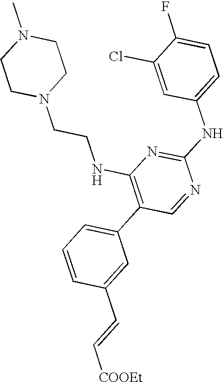 ethyl (2E)-3-[3-(2- [(3-chloro-4- fluorophenyl)amino]- 4-{[2-(4- methylpiperazin-1- yl)ethyl]amino}- pyrimidin-5- yl)phenyl]prop-2- enoate | MS(ES): 539 (M + 1) for C₂₈H₃₂ClFN₆O₂. ¹H NMR (400 MHz, DMSO-d₆): δ 1.25 (t, J = 7.08 Hz, 3H), 2.11 (s, 3H), 2.20-2.40 (m, 8H), 3.30 (m, 2H), 3.45-3.49 (m, 2H), 4.19 (q, J = 7.12 Hz, 2H), 6.46 (t, J = 4.88 Hz, 1H), 6.70 (d, J = 16.00 Hz, 1H), 7.28 (t, J = 9.12 Hz, 1H), 7.45 (d, J = 7.64 Hz, 1H), 7.51 (t, J = 7.48 Hz, 1H), 7.64-7.66 (m, 1H), 7.67 (s, 1H), 7.71-7.74 (m, 2H), 7.85 (s, 1H), 8.19 (dd, J = 2.52, 6.90 Hz, 1H), 8.31 (s, 1H), 9.41 (s, 1H). | 5-bromo-N²- (3-chloro-4- fluorophenyl)- N⁴-[2-(4- methylpiperazin- 1- yl)ethyl]- pyrimidine-2,4- diamine (Intermediate 92) |
| 199 | 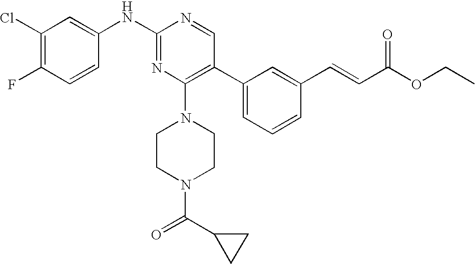 (E)-ethyl 3-(3-(2- (3-chloro-4- fluorophenylamino)- 4-(4- (cyclopropanecar- bonyl)piperazin-1- yl)pyrimidin-5- yl)phenyl)acrylate | MS(ES): 550 (M + 1) for C₂₉H₂₉ClFN₅O₃ ¹H NMR (300 MHz, DMSO-d₆) δ ppm 0.56-0.78 (m, 4 H) 1.26 (t, J = 7.06 Hz, 3 H) 1.76-2.06 (m,1 H) 3.12-3.33 (m, 4 H) 3.38-3.81 (m, 4 H) 4.20 (q, J = 7.10 Hz, 2 H) 6.74 (d, J = 16.01 Hz, 1 H) 7.34 (t, J = 9.14 Hz, 1 H) 7.42-7.60 (m, 2 H) 7.59-7.78 (m, 3 H) 7.82 (s, 1 H) 8.04-8.23 (m, 2 H) 9.65 (s, 1 H) | (4-(5-bromo- 2-(3-chloro-4- fluorophenyl- amino)- pyrimidin-4- yl)piperazin-1- yl)(cyclopropyl)- methanone (Intermediate 96) |
| 200 | 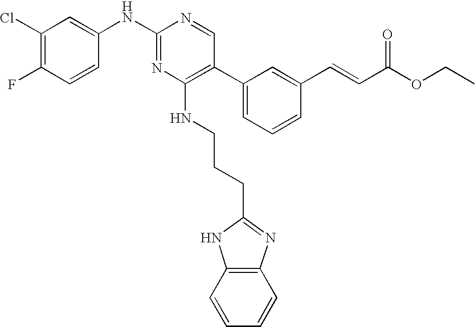 (E)-ethyl 3-(3-(4- (3-(1H- benzo[d]imidazol- 2-yl)propylamino)- 2-(3-chloro-4- fluorophenylamino) pyrimidin-5- yl)phenyl)acrylate | MS(ES): 571 (M + 1) for C₃₁H₂₈ClFN₆O₂ | N⁴-(3-(1H- benzo[d]- imidazol-2- yl)propyl)-5- bromo-N²-(3- chloro-4- fluorophenyl)- pyrimidine-2,4- diamine (Intermediate 93) |
| 201 | 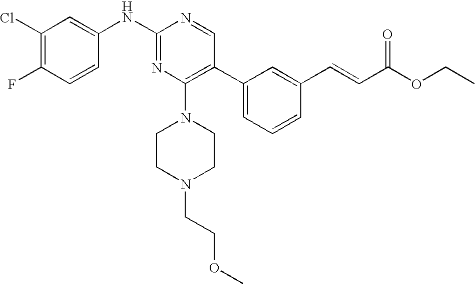 (E)-ethyl 3-(3-(2- (3-chloro-4- fluorophenylamino)- 4-(4-(2- methoxyethyl)- piperazin-1- yl)pyrimidin-5- yl)phenyl)acrylate | MS(ES): 540 (M + 1) for C₂₈H₃₁ClFN₅O₃ ¹H NMR (300 MHz, DMSO-d₆) δ ppm 1.26 (t, 3 H) 2.30-2.47 (m, 6 H) 3.10-3.28 (m, 7 H) 3.39 (t, J = 5.75 Hz, 2 H) 4.20 (q, J = 7.10 Hz, 2 H) 6.72 (d, J = 16.01 Hz, 1 H) 7.33 (t, J = 9.14 Hz, 1 H) 7.42- 7.55 (m, 2 H) 7.55-7.75 (m, 3 H) 7.81 (s, 1 H) 8.06 (s, 1 H) 8.19 (dd, J = 6.88, 2.54 Hz, 1 H) 9.61 (s, 1 H) | 5-bromo-N-(3- chloro-4- fluorophenyl)- 4-[4-(2- methoxyethyl) piperazin-1- yl]pyrimidin- 2-amine (Intermediate 108) |
| 202 | 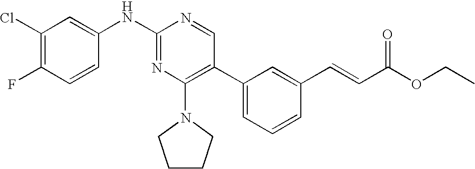 (E)-ethyl 3-(3-(2- (3-chloro-4- fluorophenylamino)- 4-(pyrrolidin-1- yl)pyrimidin-5- yl)phenyl)acrylate | MS(ES): 467 (M + 1) for C₂₅H₂₄ClFN₄O₂ ¹H NMR (300 MHz, DMSO-d₆) δ ppm 1.26 (t, J = 7.06 Hz, 3 H) 1.76 (br. s., 4 H) 3.17 (br. s., 4 H) 4.19 (q, J = 7.16 Hz, 2 H) 6.71 (d, J = 16.01 Hz, 1 H) 7.20-7.39 (m, 2 H) 7.44 (t, J = 7.91 Hz, 1 H) 7.55- 7.81 (m, 4 H) 7.88 (s, 1 H) 8.26 (dd, J = 6.97, 2.64 Hz, 1 H) 9.46 (s, 1 H) | 5-bromo-N-(3- chloro-4- fluorophenyl)- 4-(pyrrolidin- 1- yl)pyrimidin- 2-amine (Intermediate 104) |

### Example 203

### Ethyl 5-55 2-[(3-chloro-4-fluorophenyl)amino]-4-[4-(pyridin-4-yl)-1H-pyrazol-1-yl]pyrimidin-5-yl}pyridine-3-carboxylate

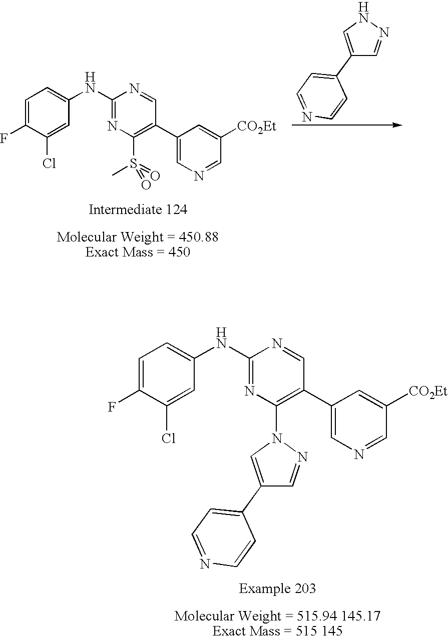

A solution of 4-(1H-pyrazol-4-yl)-pyridine (838 mg, 5.78 mmol) in DMF (2 mL) was added slowly to a suspension of sodium hydride (60%, 220 mg, 5.52 mmol) in DMF (2 mL). The reaction mixture was stirred for 25 min at room temperature. A solution of ethyl 5-{2-[(3-chloro-4-fluorophenyl)amino]-4-(methylsulfonyl)pyrimidin-5-yl}pyridine-3-carboxylate (Intermediate 124, 1.18 g, 2.63 mmol) in DMF (1 mL) was added slowly to the reaction mixture and the mixture was stirred for 1 h. Water was added (˜6 mL), and the solid formed was filtered, dried to yield the title compound (900 mg).

MS(ES): 516 (M+1) for C₂₆H₁₉ClFN₇O₂.

¹H NMR (400 MHz, DMSO-d₆): δ 1.30 (t, J=7.20 Hz, 3H), 4.34 (q, J=7.20 Hz, 2H), 7.46 (t, J=8.80 Hz, 1H), 7.70-7.76 (m, 3H), 8.14-8.16 (m, 2H), 8.27 (s, 1H), 8.59 (s, 2H), 8.71 (s, 1H), 8.76 (s, 1H), 9.03 (s, 1H), 9.09 (s, 1H), 10.41 (s, 1H).

The following examples were prepared using the general method described above for Example 203 using ethyl 5-{2-[(3-chloro-4-fluorophenyl)amino]-4-(methylsulfonyl)pyrimidin-5-yl}pyridine-3-carboxylate (Intermediate 124), sodium hydride and the starting material (SM) indicated.

| Ex | Compound | Data | SM |
|---|---|---|---|
| 204 | 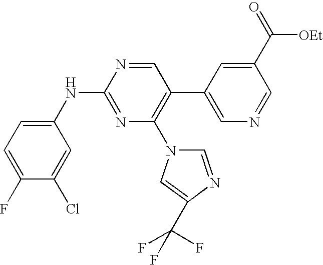 ethyl 5-{2-[(3-chloro- 4- fluorophenyl)amino]- 4-[4- (trifluoromethyl)-1H- imidazol-1- yl]pyrimidin-5- yl}pyridine-3- carboxylate | MS(ES): 507 (M + 1) for C₂₂H₁₅ClF₄N₆O₂. ¹H NMR (400 MHz, DMSO-d₆): δ 1.31 (t, J = 7.12 Hz, 3H), 4.33 (q, J = 7.08 Hz, 2H), 7.42 (t, J = 9.12 Hz, 1H), 7.70 (ddd, J = 2.72, 4.14, 9.07 Hz, 1H), 7.92 (d, J = 1.24 Hz, 1H), 8.02 (s, 1 H), 8.06 (dd, J = 2.52, 6.68 Hz, 1H), 8.11 (t, J = 2.12 Hz, 1H), 8.69 (d, J = 2.12 Hz, 1H), 8.91 (s, 1H), 9.07 (d, J = 1.88 Hz, 1H), 10.50 (s, 1H). | 4- Trifluoro- methyl-1H- imidazole |
| 205 | 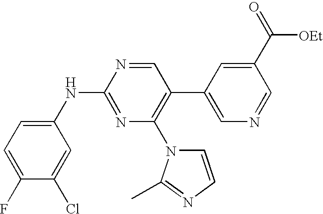 ethyl 5-{2-[(3-chloro- 4- fluorophenyl)amino]- 4-(2-methyl-1H- imidazol-1- yl)pyrimidin-5- yl}pyridine-3- carboxylate | MS(ES): 453 (M + 1) for C₂₂H₁₈ClFN₆O₂. ¹H NMR (300 MHz, DMSO-d₆): δ 1.33 (t, J = 6.93 Hz, 3H), 2.20 (s, 3H), 4.3 (q, J = 6.66 Hz, 2H), 6.80 (s, 1H), 7.04 (s, 1H), 7.41 (t, J = 8.85 Hz, 1H), 7.66 (m, 1H), 7.98 (s, 1H), 8.06 (d, J = 4.36 Hz, 1H)), 8.52 (s, 1H), 8.90 (s, 1H), 8.97 (s, 1H), 10.42 (s, 1H). | 2-Methyl- 1H-imidazole |
| 206 | 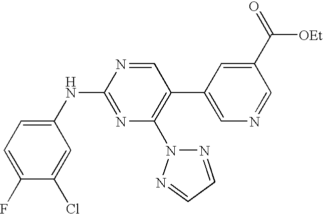 ethyl 5-{2-[(3-chloro- 4- fluorophenyl)amino]- 4-(2H-1,2,3-triazol-2- yl)pyrimidin-5- yl}pyridine-3- carboxylate | MS(ES): 440 (M + 1) for C₂₀H₁₅ClFN₇O₂. ¹H NMR (400 MHz, DMSO-d₆): δ 1.32 (t, J = 7.20 Hz, 3H), 4.34 (q, J = 7.20 Hz, 2H), 7.43 (t, J = 9.20 Hz, 1H), 7.76-7.80 (m, 1H), 8.02 (t, J = 2.00 Hz, 1H), 8.12 (br s, 2H), 8.24 (d, J = 4.40 Hz, 1H), 8.62 (d, J = 2.00 Hz, 1H), 8.88 (s, 1H), 9.03 (d, J = 1.60 Hz, 1H), 10.59 (br s, 1H). | 1H-[1,2,3]Triazole |
| 207 | 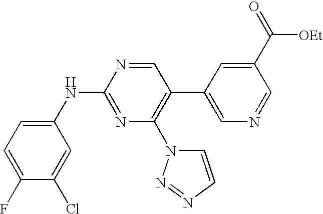 ethyl 5-{2-[(3-chloro- 4- fluorophenyl)amino]- 4-(1H-1,2,3-triazol-1- yl)pyrimidin-5- yl}pyridine-3- carboxylate | MS(ES): 440 (M + 1) for C₂₀H₁₅ClFN₇O₂. ¹H NMR (400 MHz, DMSO-d₆): δ 1.32 (t, J = 7.20 Hz, 3H), 4.35 (q, J = 7.20 Hz, 2H), 7.44 (t, J = 8.80 Hz,1H), 7.72-7.76 (m, 1H), 7.96 (d, J = 1.16 Hz, 1H), 8.06 (m, 1H), 8.08 (t, J = 2.12 Hz, 1H), 8.64-8.65 (m, 2H), 8.90 (s, 1H), 9.04 (d, J = 1.60 Hz, 1H), 10.54 (s, 1H). | 1H- [1,2,3]Triazole |
| 208 | 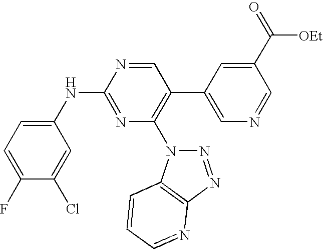 ethyl 5-{2-[(3-chloro- 4- fluorophenyl)amino]- 4-(1H- [1,2,3]triazolo[4,5- b]pyridin-1- yl)pyrimiddin-5- yl}pyridine-3- carboxylate + 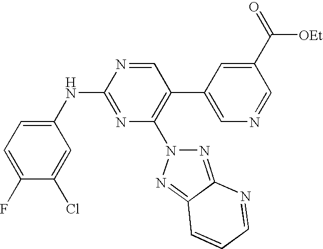 ethyl 5-{2-[(3-chloro- 4- fluorophenyl)amino]- 4-(2H- [1,2,3]triazolo[4,5- b]pyridin-2- yl)pyrimidin-5- yl}pyridine-3- carboxylate | MS(ES): 491 (M + 1) for both isomers Mixture of regioisomers (3:2) ¹H NMR (400 MHz, DMSO-d₆): δ 1.22 (t, J = 7.08 Hz, 3H), 1.29 (t, J = 7.04 Hz, 3H), 4.24-4.26 (m, 2H), 4.32 (q, J = 7.04 Hz, 2H), 7.44 (t, J = 9.04 Hz, 1H), 7.80- 7.83 (m, 1H), 7.60-7.70 (m, 1H), 8.13 (dd, J = 2.28, 6.76 Hz, 1H), 8.23-8.25 (m, 1H), 8.50 (dd, J = 1.44, 8.76 Hz, 1H), 8.62 (d, J = 2.20 Hz, 1H), 8.77-8.86 (m, 2H), 9.00 (d, J = 1.92 Hz, 1H), 9.07 (s, 1H),10.51 (br s, 1H), 10.76 (br s, 1H). | 1H- [1,2,3]Triazolo- [4,5- b]pyridine |
| 209 | 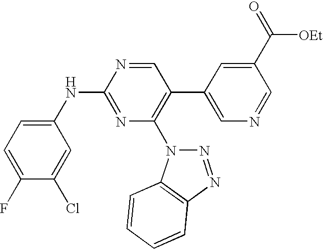 ethyl 5-{4-(1H- benzotriazol-1-yl)-2- [(3-chloro-4- fluorophenyl)amino]- pyrimidin-5- yl}pyridine-3- carboxylate | MS(ES): 490 (M + 1) for C₂₄H₁₇ClFN₇O₂. ¹H NMR (400 MHz, DMSO-d₆): δ 1.19-1.30 (m, 3H), 4.23-4.33 (m, 2H), 7.43 (t, J = 9.08 Hz, 1H), 7.51-7.58 (m, 1H), 7.64-7.66 (m, 1H), 7.72-7.76 (m, 1H), 7.91-7.94 (m, 1H), 8.14-8.23 (m, 3H), 8.71 (s, 1H), 8.98 (s, 1H), 9.03-9.04 (m, 1H), 10.48 (s, 1H). | 1H- Benzotriazole |

### Example 210

### ethyl 5-[4-(1H-benzimidazol-2-ylmethylamino)-2-[(3-chloro-4-fluorophenyl)amino]pyrimidin-5-yl]pyridine-3-carboxylate hydrochloride

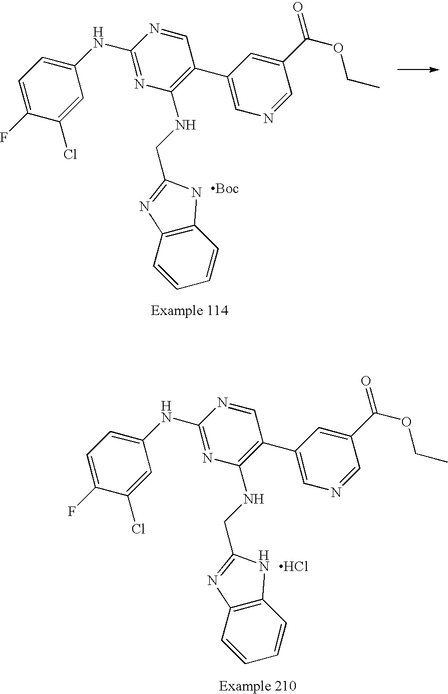

To a stirred solution of tert-butyl 2-[[[2-[(3-chloro-4-fluorophenyl)amino]-5-(5-ethoxycarbonylpyridin-3-yl)pyrimidin-4-yl]amino]methyl]benzimidazole-1-carboxylate (Example 114, 500 mg, 0.81 mmol) in 1,4-dioxane (10 mL) under nitrogen atmosphere was added 4N hydrochloric acid in 1,4-dioxane (10 mL) dropwise. The reaction mixture was stirred at room temperature for 24 h, then concentrated to give ethyl 5-[4-(1H-benzimidazol-2-ylmethylamino)-2-[(3-chloro-4-fluorophenyl)amino]pyrimidin-5-yl]pyridine-3-carboxylate hydrochloride as a white solid in 37% yield (150 mg, 0.29 mmol).

MS(ES):518.2 (M+1) for C₂₆H₂₁ClFN₇O₂.

¹H NMR (400 MHz) DMSO-d₆: δ 1.35 (t, J=7.08 Hz, 3H), 4.40 (q, J=2.88 Hz, 2H), 4.98 (d, J=5.16 Hz, 2H), 7.12 (t, J=0.00 Hz, 1H), 7.45 (br 1H), 7.52 (dd, J=6.12, 3.20 Hz, 1H), 7.60 (s 7.77 (dd, J=6.16, 3.12 Hz, 2H), 8.09 (s, 1H), 8.49 (t, J=2.08 Hz, 1H), 9.03 (d, J=2.20 Hz, 1H), 9.13 (d, J=2.00 Hz, 1H), 10.00 (br s, 1H).

The following examples were prepared by the general method described above for Example 210 using 4N hydrochloric acid in dioxane and the starting material (SM) indicated.

| Ex | Compound | Data | SM |
|---|---|---|---|
| 211 | 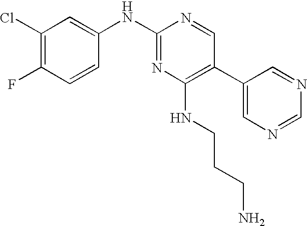 N⁴-(3-aminopropyl)- N²-(3-chloro-4- fluorophenyl)-5,5′- bipyrimidine-2,4- diamine | MS(ES): 374 (M + 1) for C₁₇H₁₇ClFN₇ ¹H NMR (300 MHz, DMSO-d6) δ ppm 3.14-3.37 (m, 4 H) 3.75- 3.98 (m, 4 H) 7.27 (d, J = 3.77 Hz, 1 H) 7.34 (t, J = 9.14 Hz, 1H) 7.48- 7.65 (m, 1 H) 7.69 (d, J = 3.77 Hz, 1 H) 8.10 (dd, J = 6.88, 2.54 Hz, 1 H) 8.21 (s, 1 H) 9.82 (s, 1 H) 13.10 (s, 1 H) | tert-butyl 3- (2-(3-chloro- 4- fluorophenyl amino)-5,5′- bipyrimidin- 4- ylamino)- propyl- carbamate (Example 74) |

### Example 212

### Methyl 2-(2-(3-chloro-4-fluorophenylamino)-4-(3-methoxypropylamino)pyrimidin-5-yl)thiazole-4-carboxylate

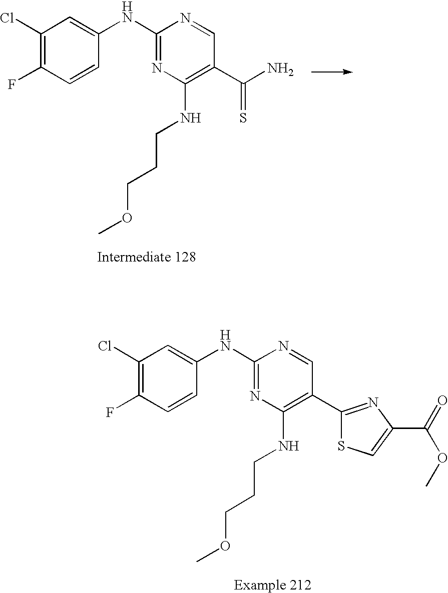

A stirred suspension of 2-(3-chloro-4-fluorophenylamino)-4-(3-methoxypropylamino)pyrimidine-5-carbothioamide (Intermediate 128, 171 mg, 0.46 mmol) and methyl 3-bromo-2-oxopropanoate (84 mg, 0.46 mmol) in ethanol (2 mL) was purged with a stream of nitrogen and then placed under an atmosphere of nitrogen. This was heated to 80 degrees C. for several days, with ethanol replaced as necessary. The reaction mixture was allowed to cool to room temperature. The mixture was diluted with dimethylsulfoxide (5 mL). The title compound was isolated (80 mg, 38%) via reverse-phase chromatography (acetonitrile/water/ammonium acetate).

MS: ES+452 for C₁₉H₁₉ClFN₅O₃S.

¹H NMR (300 MHz, DMSO-d₆) δ ppm 1.89 (quin, J=6.45 Hz, 2 H) 3.27 (s, 3 H) 3.51 (t, J=6.12 Hz, 2H) 3.63 (q, J=6.40 Hz, 2H) 3.87 (s, 3H) 7.33 (t, J=9.04 Hz, 1H) 7.57-7.71 (m, 1H) 8.24 (dd, J=6.88, 2.35 Hz, 1H) 8.45 (s, 1H) 8.60 (s, 1H) 9.37 (t, J=5.09 Hz, 1H) 9.88 (s, 1H).

### Example 213

### 4-(azepan-1-yl)-N-(3-chloro-4-fluorophenyl)-5-pyrimidin-5-ylpyrimidin-2-amine

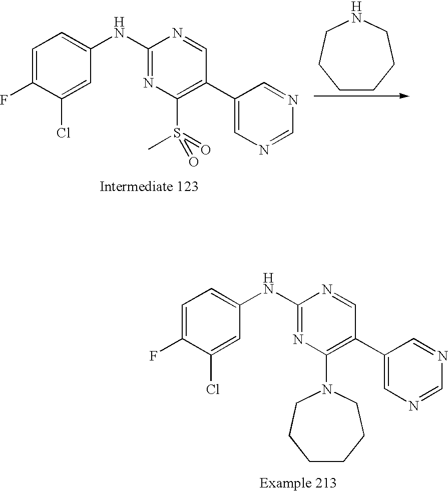

N-(3-Chloro-4-fluorophenyl)-4-methylsulfonyl-5-pyrimidin-5-ylpyrimidin-2-amine (Intermediate 123, 0.21 mmol, 80 mg) was suspended in NMP (1 mL), then treated with N,N-diisopropylethylamine (0.25 mmol, 32 mg) and hexamethyleneimine (2.63 mmol). The mixture was heated at 90° C. for 30 min in a sealed tube. The reaction mixture was added to water and stirred for 15 min. The precipitated solid was filtered, washed with water and dried to afford the crude product which was further purified by flash chromatography to yield 18 mg of the title compound (0.045 mmol, 21%).

MS(ES):399 (M+1) for C₂₀H₂₀ClFN₆.

¹H NMR 400 MHz DMSO-d₆: δ 1.41 (br s, 4H), 1.62 (br s, 4H), 3.31 (br s, 4H), 7.32 (t, J=9.12 Hz, 1H), 7.56 (ddd, J=9.00, 4.06, 2.76 Hz, 1H), 7.93 (s, 1H), 8.26 (dd, J=6.90, 2.52 Hz, 1H), 8.80 (s, 2H), 9.10 (s, 1H), 9.57 (s, 1H).

### Example 214

### 5-(2-(3-chloro-4-fluorophenylamino)-4-(3-(dimethylamino)propylamino)pyrimidin-5-yl)nicotinic acid

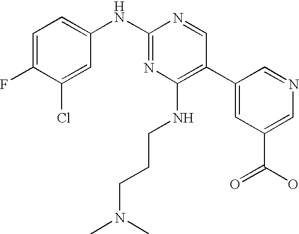

A solution of ethyl 5-(2-(3-chloro-4-fluorophenylamino)-4-(3-(dimethylamino)propylamino)pyrimidin-5-yl) nicotinate (Example 5, 50 mg, 0.09 mmol) in methanol (0.3 ml) was stirred under ambient conditions; if solubility was less than complete to the naked eye then small volumes of THF were added as necessary. Sodium hydroxide (aqueous, 1 N, 0.341 ml) was added to the solution, which was stirred under ambient conditions until high or complete conversion was indicated by LCMS or TLC. Careful acidification with 1 N HCl (aq) was followed by an aqueous workup, using methylene chloride and methanol (9:1) as the organic phase to extract the water layer (4×25 ml). The organic extracts were combined, dried over sodium sulfate, and concentrated, affording product of high purity (15 mg) which was characterized by LCMS and ¹H NMR.

MS: ES+445 for C₂₁H₂₂ClFN₆O₂

¹H NMR (300 MHz, DMSO-D6) δ ppm 1.85-2.01 (m, 2H) 2.60 (s, 6H) 2.93 (t, J=7.06 Hz, 2H) 3.36-3.52 (m, 2H) 7.14 (t, J=5.18 Hz, 1H) 7.32 (t, J=9.14 Hz, 1H) 7.58-7.67 (m, 1H) 7.85 (s, 1H) 8.17 (t, J=2.07 Hz, 1H) 8.21 (dd, J=6.97, 2.64 Hz, 1H) 8.69 (d, J=2.07 Hz, 1H) 8.88 (d, J=1.51 Hz, 1H) 9.50 (s, 1H)

The following examples were prepared using the general method described above for Example 214 using 1N sodium hydroxide and the starting material (SM) indicated.

| Ex | Compound | Data | SM |
|---|---|---|---|
| 215 | 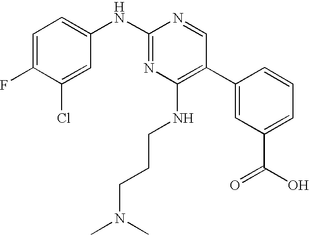 3-(2-[(3-chloro-4- fluorophenyl)amino]-4- {[3-(dimethylamino) propyl]amino} pyrimidin-5-yl)benzoic acid | MS: ES+ 444 for C₂₂H₂₃ClFN₅O₂ ¹H NMR (300 MHz, DMSO- D6) δ ppm1.89-2.05 (m, 2 H) 2.69 (d, J = 4.71 Hz, 6 H) 2.93- 3.06 (m, 2 H) 3.36-3.48 (m, 2 H) 7.40-7.75 (m, 4 H) 7.83- 8.26 (m, 5 H) 10.69 (d, J = 83.08 Hz, 2 H) 13.17 (s, 1 H) | methyl 3-(2-(3- chloro-4- fluorophenyl- amino)-4-(3- (dimethylamino) propylamino) pyrimidin-5- yl)benzoate (Example 2_ |
| 216 | 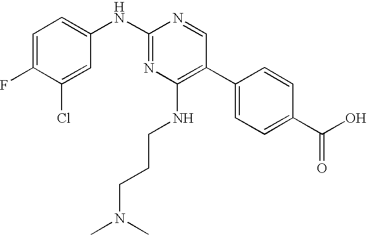 4-(2-[(3-chloro-4- fluorophenyl)amino]-4- {[3-(dimethylamino) propyl]amino} pyrimidin-5-yl)benzoic acid | MS: ES+ 444 for C₂₂H₂₃ClFN₅O₂ ¹H NMR (300 MHz, DMSO- D6) δ ppm 1.89-2.03 (m, 2 H) 2.69 (s, 6 H) 2.97-3.08 (m, 2 H) 3.40-3.51 (m, 2 H) 6.92 (t, J = 5.56 Hz, 1 H) 7.33 (t, J = 9.14 Hz, 1 H) 7.53 (d, J = 8.48 Hz, 2 H) 7.59-7.69 (m, 1 H) 7.86 (s, 1 H) 8.00 (d, J = 8.48 Hz, 2 H) 8.19 (dd, J = 6.97, 2.64 Hz, 1 H) 9.51 (s, 1 H) | Ethyl 4-(2-(3- chloro-4- fluorophenyl- amino)-4-(3- (dimethylamino) propylamino) pyrimidin-5- yl) benzoate (Example 6) |
| 217 | 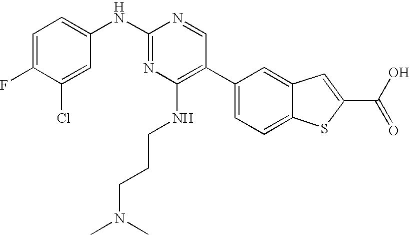 5-(2-(3-chloro-4- fluorophenylamino)-4-(3- (dimethylamino) propylamino) pyrimidin-5-yl)benzo[b] thiophene-2-carboxylic acid | MS: ES+ 500 for C₂₄H₂₃ClFN₅O₂S ¹H NMR (300 MHz, DMSO-d₆) d ppm 1.66 (dq, J = 6.59, 6.41 Hz, 2 H) 1.90 (s, 6 H) 2.27 (t, J = 6.22 Hz, 2 H) 3.40-3.48 (m, 2 H) 7.16-7.34 (m, 3 H) 7.54 (s, 1 H) 7.59-7.69 (m, 1 H) 7.72-7.81 (m, 2 H) 7.91 (d, J = 8.29 Hz, 1 H) 8.26 (dd, J = 6.88, 2.54 Hz, 1 H) 9.35 (s, 1 H) | Methyl 5-{2- (3-chloro-4- fluorophenyl amino)-4-[3- (dimethylamino) propylamino] pyrimidin-5- yl} benzo[b]thio- phene-2- carboxylate (Example 22) |
| 218 | 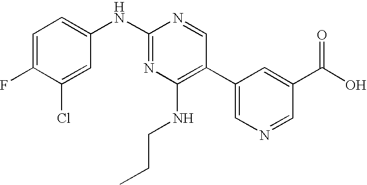 5-[2-(3-Chloro-4-fluoro- phenylamino)-4- propylamino-pyrimidin- 5-yl]-nicotinic acid | MS(ES): 402 (M + 1) for C₁₉H₁₇ClFN₅O₂. ¹H NMR (400 MHz, DMSO- d₆): δ 0.87 (t, J = 7.44 Hz, 3H), 1.56 (q, J = 7.36 Hz, 2H), 3.31 (q, J = 6.12 Hz, 3H), 7.43-7.50 (m, 2H), 7.92 (s, 1H), 8.05 (dd, J = 6.74, 2.36 Hz, 1H), 8.25 (t, J = 2.12 Hz, 2H), 8.78 (d, J = 2.2 Hz, 1H), 9.12 (d, J= 1.96 Hz, 1H), 10.56 (br s, 1H). | 5-[2-(3- Chloro-4- fluoro- phenylamino)- 4- propylamino- pyrimidin-5- yl]-nicotinic acid ethyl ester (Example 90) |
| 219 | 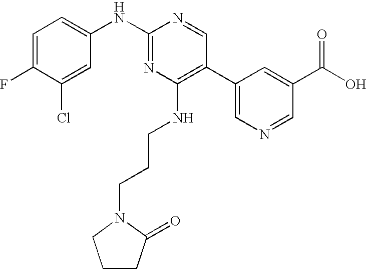 5-{2-(3-Chloro-4-fluoro- phenylamino)-4-[3-(2- oxo-pyrrolidin-1-yl)- propylamino]-pyrimidin- 5-yl}-nicotinic acid | MS(ES): 485 (M + 1) for C₂₃H₂₂ClFN₆O₃. ¹H NMR (400 MHz, DMSO- d₆): δ 1.74 (t, J = 6.88 Hz, 2H), 1.86 (m, 2H), 2.15 (t, J = 8.04 Hz, 2H), 3.19-3.22 (m, 6H), 6.91 (br s, 1H), 7.32 (t, J = 9.16 Hz, 1H), 7.63 (m, 1H), 7.84 (s, 1H), 8.19 (s, 1H), 8.76 (s, 1H), 9.02 (s, 1H), 9.48 (s, 1H). | 5-{2-(3- Chloro-4- fluoro- phenylamino)- 4-[3-(2-oxo- pyrrolidin-1- yl)- propylamino]- pyrimidin-5- yl}-nicotinic acid ethyl ester (Example 91) |
| 220 | 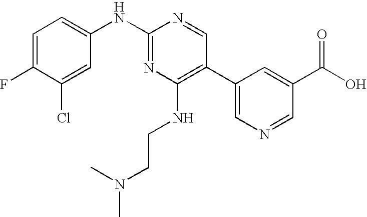 5-[2-(3-Chloro-4-fluoro- phenylamino)-4-(2- dimethylamino- ethylamino)-pyrimidin-5- yl]-nicotinic acid | MS(ES): 431 (M + 1) for C₂₀H₂₀ClFN₆O₂. ¹H NMR (400 MHz, DMSO- d₆): δ 2.77 (s, 6H), 3.28-3.32 (m, 2H), 3.67 (br s, 2H), 7.07 (t, J = 4.8 Hz, 1H), 7.34 (t, J = 9.04 Hz, 1H), 7.65 (m, 1H), 7.92 (s, 1H), 8.09 (dd, J = 6.7, 2.48 Hz, 1H), 8.23 (br s, 1H), 8.82 (d, J = 1.96 Hz, 1H), 9.03 (d, J = 1.72 Hz, 1H), 9.55 (s, 1H). | 5-[2-(3- Chloro-4- fluoro- phenylamino)- 4-(2- dimethylamino- ethylamino)- pyrimidin-5- yl]-nicotinic acid ethyl ester (Example 93) |
| 221 | 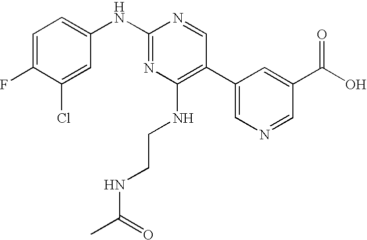 5-[4-(2-Acetylamino- ethylamino)-2-(3-chloro- 4-fluoro-phenylamino)- pyrimidin-5-yl]-nicotinic acid | MS(ES): 445 (M + 1) for C₂₀H₁₈ClFN₆O₃. ¹H NMR (400 MHz, DMSO- d₆): δ 1.79 (s, 3H),6.98 (br s, 1H), 7.3 (t, J = 9.16 Hz, 1H), 7.67 (m, 1H), 7.85 (s, 1H), 7.91 (s, 1H), 8.15 (dd, J = 6.86, 2.52 Hz, 1H), 8.22 (s, 1H), 8.77 (s, 1H), 9.03 (s, 1H), 9.49 (s, 1H). | 5-[4-(2- Acetylamino- ethylamino)- 2-(3-chloro- 4-fluoro- phenylamino)- pyrimidin-5- yl]-nicotinic acid ethyl ester (Example 94) |
| 222 | 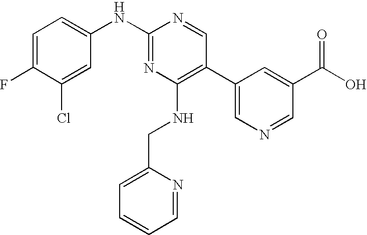 5-{2-(3-Chloro-4-fluoro- phenylamino)-4- [(pyridin-2-ylmethyl)- amino]-pyrimidin-5-yl]- nicotinic acid | MS(ES): 449 (M − 1) and 451 (M + 1) for C₂₂H₁₆ClFN₆O₂. ¹H NMR (400 MHz, DMSO- d₆): δ 4.67 (d, J = 5.8 Hz, 2H), 7.16 (t, J = 9.12 Hz, 1H), 7.23 (t, J = 1.64 Hz, 1H), 7.32 (d, J = 7.88 Hz, 1H), 7.48-7.55 (m, 2H), 7.70-7.74 (m, 1H), 7.91- 7.93 (m, 2H), 8.31 (t, J = 2.08 Hz, 1H), 8.51 (dd, J = 4.8, 0.76 Hz, 1H), 8.86 (d, J = 2.24 Hz, 1H), 9.05 (d, J = 1.96 Hz, 1H), 9.42 (s, 1H). | 5-{2-(3- Chloro-4- fluoro- phenylamino)- 4-[(pyridin- 2-ylmethyl)- amino]- pyrimidin-5- yl}-nicotinic acid ethyl ester (Example 95) |
| 223 | 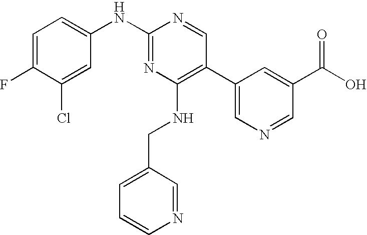 5-{2-(3-Chloro-4-fluoro- phenylamino)-4- [(pyridin-3-ylmethyl)- amino]-pyrimidin-5-yl}- nicotinic acid | MS(ES): 449.1 (M − 1) for C₂₂H₁₆ClFN₆O₂. ¹H NMR (400 MHz, DMSO- d₆): δ 4.60 (d, J = 5.76 Hz, 2H), 7.22 (t, J = 9.04 Hz, 1H), 7.31-7.34 (m, 1H), 7.5-7.56 (m, 2H), 7.73 (d, J = 8.08 Hz, 1H), 7.90 (s, 1H), 8.02 (dd, J = 6.72, 2.24 Hz, 1H), 8.24 (br s, 1H), 8.41 (br s,1H), 8.56 (br s, 1H), 8.80 (br s, 1H), 9.04 (br s, 1H), 9.46 (br s, 1H). | 5-{2-(3- Chloro-4- fluoro- phenylamino)- 4-[(pyridin- 3-ylmethyl)- amino]- pyrimidin-5- yl}-nicotinic acid ethyl ester (Example 96) |
| 224 | 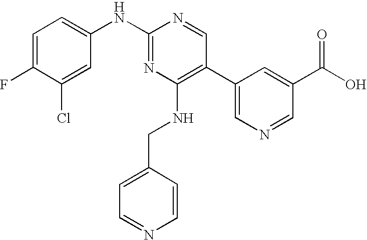 5-{2-(3-Chloro-4-fluoro- phenylamino)-4- [(pyridin-4-ylmethyl)- amino]-pyrimidin-5-yl}- nicotinic acid | MS(ES): 451 (M + 1) for C₂₂H₁₆ClFN₆O₂. ¹H NMR (400 MHz, DMSO- d₆): δ 4.65 (d, J = 6.04 Hz, 2H), 7.20 (t, J = 9.08 Hz, 1H), 7.42 (dd, J = 7.66, 3.44 Hz, 1H), 7.53 (d, J = 5.04 Hz, 1H),7.71 (br s, 1H), 7.86 (d, J = 6.00 Hz,1H), 7.95 (s, 1H), 8.30 (t, J = 2.16 Hz, 1H), 8.59 (d, J = 6.12 Hz, 2H), 8.87 (d, J = 2.24 Hz, 1H),9.07 (d, J = 2.00 Hz, 1H), 9.56 (br s, 1H). | 5-{2-(3- Chloro-4- fluoro- phenylamino)- 4-[(pyridin- 4-ylmethyl)- amino]- pyrimidin-5- yl}-nicotinic acid ethyl ester (Example 97) |
| 225 | 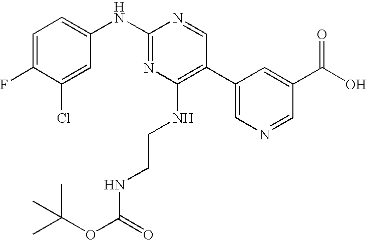 5-[4-(2-tert- Butoxycarbonylamino- ethylamino)-2-(3-chloro- 4-fluoro-phenylamino)- pyrimidin-5-yl]-nicotinic acid | MS(ES): 503.1 (M + 1) for C₂₃H₂₄ClFN₆O₄ ¹H NMR (400 MHz, DMSO- d₆): δ 1.31 (s, 9H), 3.19 (br s, 2H), 3.39 (br s, 2H), 6.85 (m, 1H), 6.91 (m, 1H), 7.31 (t, J = 9.08 Hz, 1H), 7.68 (br s, 1H), 7.84 (s, 1H), 8.14 (d, J = 5.16 Hz, 2H) 8.18 (s, 1H), 8.76 (s, 1H), 9.02 (d, J = 1.56 Hz, 1H), 9.47 (s, 1H). | 5-[4-(2-tert- Butoxycar- bonylamino- ethylamino)- 2-(3-chloro- 4-fluoro- phenylamino)- pyrimidin-5- yl]-nicotinic acid ethyl ester (Example 98) |
| 226 | 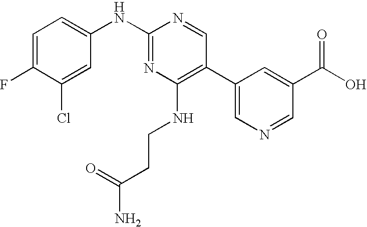 5-[4-(2-Carbamoyl- ethylamino)-2-(3-chloro- 4-fluoro-phenylamino)- pyrimidin-5-yl]-nicotinic acid | MS(ES): 431 (M + 1) for C₁₉H₁₆ClFN₆O₃. ¹H NMR (400 MHz, DMSO- d₆): δ 2.4 (t, J = 7.08 Hz, 2H), 3.55 (m, 2H), 6.8 (br s, 1H), 6.93 (m, 1H), 7.24-7.29 (m, 2H), 7.70-7.72 (m, 1H), 7.85 (s, 1H), 8.15 (m, 1H), 8.74 (s, 1H), 9.01 (s, 1H), 9.46 (s, 1H). | 5-[4-(2- Carbamoyl- ethylamino)- 2-(3-chloro- 4-fluoro- phenylamino)- pyrimidin-5- yl]-nicotinic acid ethyl ester (Example 99) |
| 227 | 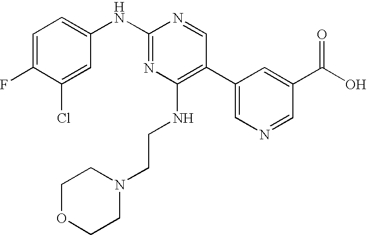 5-[2-(3-Chloro-4-fluoro- phenylamino)-4-(2- morpholin-4-yl- ethylamino)-pyrimidin-5- yl]-nicotinic acid | MS(ES): 473 (M + 1) for C₂₂H₂₂ClFN₆O₃. ¹H NMR (400 MHz, DMSO- d₆): δ 2.36 (s, 4H), 3.49 (m, 6H), 6.56 (m, 1H), 7.28 (t, J = 8.96 Hz, 1H), 7.65-7.67 (m, 1H), 7.78 (s, 1H), 8.06 (s, 1H), 8.17 (dd, J = 6.86, 2.44 Hz, 1H), 8.45 (s, 1H), 8.91 (s, 1H), 9.40 (s, 1H). | 5-[2-(3- Chloro-4- fluoro- phenylamino)- 4-(2- morpholin-4- yl- ethylamino)- pyrimidin-5- yl]-nicotinic acid ethyl ester (Example 100) |
| 228 | 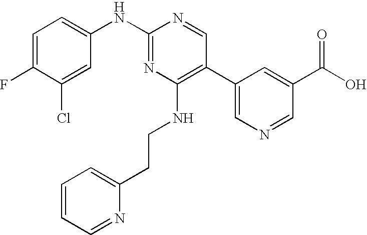 5-[2-(3-Chloro-4-fluoro- phenylamino)-4-(2- pyridin-2-yl-ethylamino)- pyrimidin-5-yl]-nicotinic acid | MS(ES): 465.2 (M) for C₂₃H₁₈ClFN₆O₂. ¹H NMR (400 MHz, DMSO- d₆): δ 3.03 (t, J = 7 Hz, 2H), 3.72 (q, J = 6.64 Hz, 2H), 7.08 (t, J = 5.68 Hz, 1H), 7.18-7.27 (m, 3H), 7.66-7.70 (m, 2H), 7.85 (s, 1H), 8.14-8.18 (m, 2H), 8.44 (d, J = 4.08 Hz, 1H), 8.71 (d, J = 1.88 Hz, 1H), 9.01 (d, J = 1.64 Hz, 1H), 9.47 (s, 1H), 13.4 (br s, 1H). | 5-[2-(3- Chloro-4- fluoro- phenylamino)- 4-(2-pyridin- 2-yl- ethylamino)- pyrimidin-5- yl]-nicotinic acid ethyl ester (Example 101) |
| 229 | 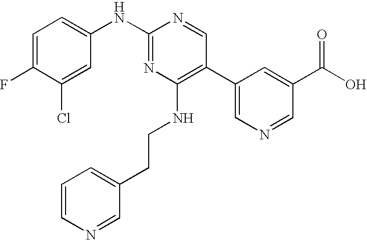 5-[2-(3-Chloro-4-fluoro- phenylamino)-4-(2- pyridin-3-yl-ethylamino)- pyrimidin-5-yl]-nicotinic acid | MS(ES): 465 (M + 1) for C₂₃H₁₈ClFN₆O₂. ¹H NMR (400 MHz, DMSO- d₆): δ 2.9 (t, J = 7 Hz,2H), 3.6 (t, J = 6.7 Hz, 2 H), 7.01 (t, J = 5.48 Hz, 1H), 7.26-7.32 (m, 2H), 7.62-7.64 (m, 2H), 7.85 (s, 1H), 8.15 (t, J = 1.96 Hz, 1H), 8.2 (dd, J = 8, 2.56 Hz, 1H), 8.40 (m, 2H), 8.68 (s, 1H), 9.01 (s, 1H), 9.47 (s 1H). | 5-[2-(3- Chloro-4- fluoro- phenylamino)- 4-(2-pyridin- 3-yl- ethylamino)- pyrimidin-5- yl]-nicotinic acid ethyl ester (Example 102) |
| 230 | 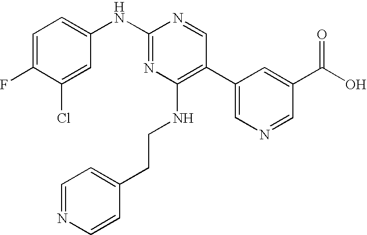 5-[2-(3-Chloro-4-fluoro- phenylamino)-4-(2- pyridin-4-yl-ethylamino)- pyrimidin-5-yl]-nicotinic acid | ¹H NMR (400 MHz, DMSO- d₆): δ 2.90 (t, J = 7.08 Hz, 2H), 3.58-3.64 (m, 2H), 7.01 (t, J = 5.40 Hz, 1H), 7.2-7.3 (m, 3H), 7.60 (m, 1H), 7.85 (s, 1H), 8.15-8.19 (m, 2H), 8.45 (dd, J = 4.48, 1.44 Hz, 2H), 8.69 (d, J = 2.20 Hz, 1H), 9.02 (d, J = 2.00 Hz, 1H), 9.48 (s, 1H). | 5-[2-(3- Chloro-4- fluoro-phenyl amino)-4-(2- pyridin-4-yl- ethylamino)- pyrimidin-5- yl]-nicotinic acid ethyl ester (Example 103) |
| 231 | 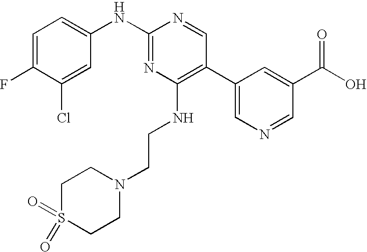 5-{2-(3-Chloro-4-fluoro- phenylamino)-4-[2-(1,1- dioxo-1λ⁶- thiomorpholin-4-yl)- ethylamino]-pyrimidin-5- yl}-nicotinic acid | MS(ES): 519 (M − 1) for C₂₂H₂₂ClFN₆O₄S ¹H NMR (400 MHz, DMSO- d₆): δ 2.68 (br s, 2H), 2.91 (br s, 4H), 3.02 (br s, 4H), 3.46 (br s, 2H), 6.78 (br s, 1H), 7.28 (m, 1H), 7.85 (m, 1H), 8.2 (m, 2H), 8.74 (s, 1H), 9 (s, 1H), 9.43 (s, 1H), 13.4 (br s, 1H). | 5-[2-(3- Chloro-4- fluoro- phenylamino)- 4-[2-(1,1- dioxo-1λ⁶- thiomorpholin- 4-yl)- ethylamino]- pyrimidin-5- yl}-nicotinic acid ethyl ester (Example 104) |
| 232 | 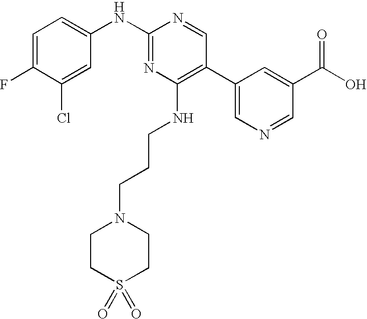 5-{2-(3-Chloro-4-fluoro- phenylamino)-4-[3-(1,1- dioxo-1λ⁶- thiomorpholin-4-yl)- propylamino]-pyrimidin- 5-yl}-nicotinic acid | MS(ES): 535 (M + 1) for C₂₃H₂₄ClFN₆O₄S. ¹H NMR (400 MHz, DMSO- d₆): δ 1.71-1.74 (t, J = 13.48 Hz,2H), 2.49-2.54 (m, 2H), 2.83 (s, 4H), 3.0 (s, 4H), 3.37- 3.42 (m, 4H), 6.95-6.97 (t, J = 4.8 Hz, 1H), 7.31 (t, J = 9.16 Hz, 1H), 7.58-7.62 (m, 1H), 7.84 (s, 1H), 8.19 (s, 1H), 8.25-8.26 (dd, J = 6.92, 2.44 Hz, 1H), 8.76 (s, 1H), 9.0 (s, 1H), 9.48 (s, 1H). | 5-{2-(3- Chloro-4- fluoro- phenylamino)- 4-[3-(1,1- dioxo-1λ⁶- thiomorpholin- 4-yl)- propylamino]- pyrimidin-5- yl}-nicotinic acid ethyl ester (Example 105) |
| 233 | 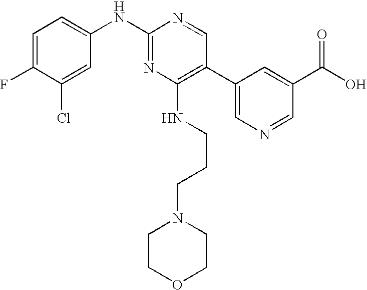 5-[2-[(3-chloro-4- fluorophenyl)amino]-4- (3-morpholin-4- ylpropylamino)pyrimidin- 5-yl]pyridine-3- carboxylic acid | MS(ES): 487 (M + 1) for C₂₃H₂₄ClFN₆O₃. ¹H NMR (400 MHz, DMSO- d₆): δ 2.00 (s, 2H), 3.05 (br s, 4H), 3.44 (d, J = 5.44 Hz, 4H), 3.79 (br s, 4H), 7.07 (s, 1H), 7.34 (t, J = 9.04 Hz, 1H), 7.64 (d, J = 8.44 Hz, 1H), 7.89 (s, 1H), 8.21 (t, J = 7.00 Hz, 2H), 8.80 (s, 1H), 9.04 (s, 1H), 9.54 (s, 1H). | 5-[2-(3- Choloro-4- fluoro-phenyl amino)-4-(3- morpholin-4- yl- propylamino)- pyrimidin-5- yl]-nicotinic acid ethyl ester (Example 106) |
| 234 | 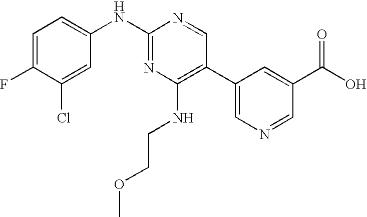 5-[2-[(3-chloro-4- fluorophenyl)amino]-4- (2- methoxyethylamino)- pyrimidin-5-yl]pyridine-3- carboxylic acid | MS(ES): 418 (M + 1) for C₁₉H₁₇ClFN₅O_{3.} ¹H NMR (400 MHz, DMSO- d₆): δ 3.25 (s, 3H), 3.49-3.54 (m, 4H), 6.92 (d, J = 5.16 Hz, 1H), 7.29 (t, J = 9.12 Hz, 1H), 7.60-7.64 (m, 1H), 7.86 (s, 1H), 8.18-8.21 (m, 2H), 8.74 (s,1H), 9.02 (s, 1H), 9.48 (s, 1H). | 5-[2-(3- Chloro-4- fluoro- phenylamino)- 4-(2- methoxy- ethylamino)- pyrimidin-5- yl]-nicotinic acid ethyl ester (Example 144) |
| 235 | 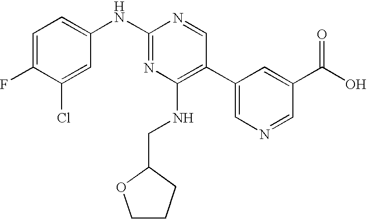 5-[2-[(3-chloro-4- fluorophenyl)amino]-4- (oxolan-2- ylmethylamino)pyrimidin- 5-yl]pyridine-3- carboxylic acid | MS(ES): 444 (M + 1) for C₂₁H₁₉ClFN₅O_{3.} ¹H NMR (400 MHz, DMSO- d₆): δ 1.59-1.62 (m, 1H), 1.78- 1.83 (m, 2H), 1.89-1.90 (m, 1H), 3.37-3.45 (m, 2H), 3.60- 3.63 (m, 1H), 3.72-4.07 (m, 1H), 4.08-4.10 (m, 1H), 6.92- 6.95 (m, 1H), 7.3 (t, J = 9.12 Hz, 1H), 7.59-7.63 (m, 1H), 7.86 (s, 1H), 8.18-8.22 (m, 2H), 8.75 (d, J = 2.12 Hz, 1H), 9.05 (d, J = 2 Hz, 1H), 9.48 (s, 1H). | ethyl 5-[2- [(3-chloro-4- fluorophenyl) amino]-4- (oxolan-2- ylmethylamino)- pyrimidin- 5- yl]pyridine- 3-carboxylate (Example 108) |
| 236 | 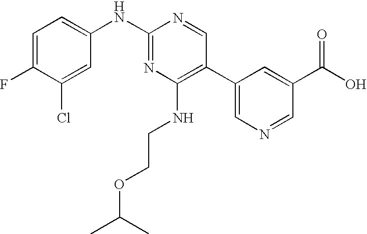 5-[2-[(3-chloro-4- fluorophenyl)amino]-4- (2-propan-2- yloxyethyolamino)pyrimidin- 5-yl]pyridine-3- carboxylic acid | MS(ES): 446.1 (M) for C₂₁H₂₁ClFN₅O₃. ¹H NMR (400 MHz, DMSO- d₆): δ 1.04 (d, J = 6.08 Hz, 6H), 3.48-3.56 (m, 5H), 6.80 (s, 1H), 7.28 (t, J = 9.12 Hz, 1H), 7.63 (ddd, J = 9.04, 4.20, 2.68 Hz, 1H), 7.85 (s, 1H), 8.16-8.20 (m, 2H), 8.68 (d, J = 1.80 Hz, 1H), 9.01 (d, J = 1.52 Hz, 1H), 9.46 (s, 1H). | ethyl 5-[2- [(3-chloro-4- fluorophenyl) amino]-4-(2- propan-2- yloxyethyl- amino)- pyrimidin- 5- yl]pyridine- 3-carboxylate (Example 109) |
| 237 | 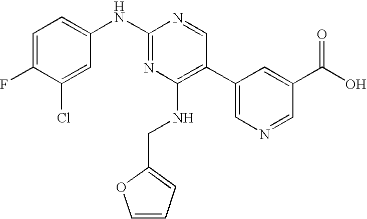 5-[2-[(3-chloro-4- fluorophenyl)amino]-4- (furan-2- ylmethylamino)pyrimidin- 5-yl]pyridine-3- carboxylic acid | MS(ES): 440 (M + 1) for C₂₁H₁₅ClFN₅O₃. ¹H NMR (400 MHz, DMSO- d₆): δ 4.6 (d, J = 5.64 Hz, 2H), 6.25 (d, J = 2.96 Hz, 1H), 6.36 (s, 1H), 7.27 (t, J = 9.16 Hz, 1H), 7.44 (t, J = 5.24 Hz, 1H), 7.55 (s, 1H), 7.57-7.61 (m, 1H), 7.90 (s, 1H), 8.13 (dd, J = 6.66, 2.28 Hz, 1H), 8.20 (s, 1H), 8.70 (d, J = 1.4 Hz, 1H), 9.03 (s, 1H), 9.49 (s, 1H). | ethyl 5-[2- [(3-chloro-4- fluorophenyl) amino]-4- (furan-2- ylmethylamino)- pyrimidin- 5- yl]pyridine- 3-carboxylate (Example 110) |
| 238 | 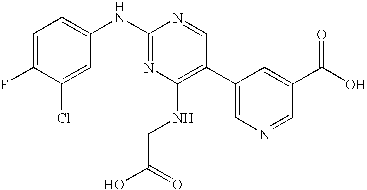 5-[4- (carboxymethylamino)-2- [(3-chloro-4- fluorophenyl)amino]- pyrimidin-5-yl]pyridine-3- carboxylic acid | MS(ES): 418 (M + 1) for C₁₈H₁₃ClFN₅O₄ ¹H NMR (400 MHz DMSO- d₆): δ 4.0 (d, J = 5.92 Hz, 2H), 7.2 (t, J = 6 Hz, 1H), 7.26 (t, J = 9.12 Hz, 1H), 7.65 (ddd, J = 9.1, 4.24, 2.6 Hz, 1H), 7.93 (s, 1H), 9.04 (d, J = 1.96 Hz), 8.03 (dd, J = 6.8, 2.43 Hz, 1H), 8.24 (t, J = 2.12 Hz, 1H), 8.8 (d, J = 2.2 Hz, 1H), 9.48 (s, 1H), 12.6 (br s, 1H), 13.6 (br s, 1H). | ethyl 5-[2- [(3-chloro-4- fluorophenyl) amino]-4-[(2- ethoxy-2- oxoethyl)- amino]- pyrimidin- 5- yl]pyridine- 3-carboxylate (Example 111) |
| 239 | 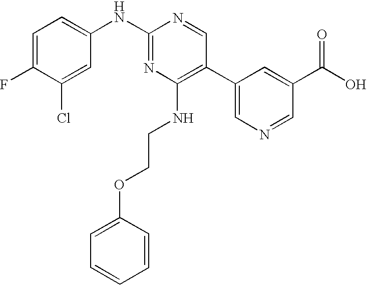 5-[2-[(3-chloro-4- fluorophenyl)amino]-4- (2- phenoxyethylamino)- pyrimidin-5-yl]pyridine-3- carboxylic acid | MS(ES): 480.1 (M + 1) for C₂₄H₁₉ClFN₅O_{3.} ¹H NMR (400 MHz DMSO- d₆): δ 3.73-3.75 (m, 2H), 4.12- 4.14 (m, 2H), 6.90-6.94 (m, 3H), 7.18 (br s, 1H), 7.22-7.27 (m, 3H), 7.6-7.7 (m, 1H), 7.89 (s, 1H), 8.18-8.20 (m, 1H), 8.22 (s, 1H), 8.76 (d, J = 2.16 Hz, 1H), 9.04 (d, J = 2 Hz, 1H), 9.54 (s, 1H), 13.55 (br s, 1H). | ethyl 5-[2- [(3-chloro-4- fluorophenyl) amino]-4-[(2- ethoxy-2- oxoethyl)- amino]- pyrimidin- 5- yl]pyridine- 3-carboxylate (Example 112) |
| 240 | 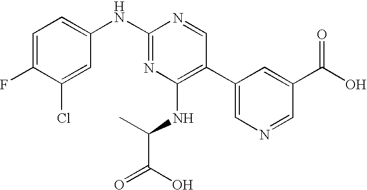 5-[2-[(3-chloro-4- fluorophenyl)amino]-4- [[(2R)-1-hydroxy-1- oxopropan-2- yl]amino]pyrimidin-5- yl]pyridine-3-carboxylic acid | MS (ESI): 432.1 (M + 1) for C₁₉H₁₅ClFN₅O₄. ¹H NMR (400 MHz, DMSO- d₆): δ 1.39 (d, J = 7.24 Hz, 3H), 4.64-4.68 (m, 1H), 6.98 (d, J = 7.34 Hz, 1H), 7.27 (d, J = 9.04 Hz, 1H), 7.64-7.68 (m, 1H), 7.91 (s, 1H), 8.02 (d, J = 4.4 Hz, 1H), 8.24 (s, 1H), 8.80 (s, 1H), 9.03 (s, 1H), 9.48 (s, 1H), 13.1 (br s, 1H). | ethyl 5-[2- [(3-chloro-4- fluorophenyl) amino]-4-[(2- ethoxy-2- oxoethyl)- amino]- pyrimidin- 5- yl]pyridine- 3-carboxylate (Example 113) |
| 241 | 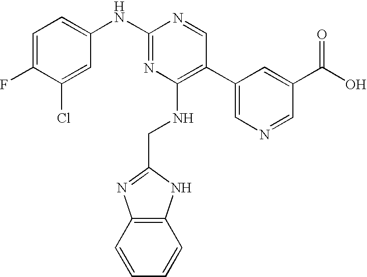 5-[4-(1H-benzimidazol- 2-ylmethylamino)-2-[(3- chloro-4- fluorophenyl)amino]- pyrimidin-5-yl]pyridine-3- carboxylic acid | MS(ES): 490.2 (M + 1) for C₂₄H₁₇ClFN₇O₂. ¹H NMR (400 MHz, DMSO- d₆): δ 4.78 (d, J = 5.56 Hz, 2H), 7.04 (t, J = 9.36 Hz, 1H), 7.10-7.12 (m, 2H), 7.49-7.54 (m, 4H), 7.85 (d, J = 3.96 Hz, 1H), 7.96 (s, 1H), 8.36 (s, 1H), 8.90 (s, 1H), 9.04 (d, J = 1.56 Hz, 1H), 9.44 (s, 1H), 12.29 (br s, 1H). | ethyl 5-[4- (1H- benzimidazol- 2- ylmethylamino)- 2-[(3- chloro-4- fluorophenyl) amino]- pyrimidin-5- yl]pyridine- 3-carboxylate hydrochloride (Example 208) |
| 242 | 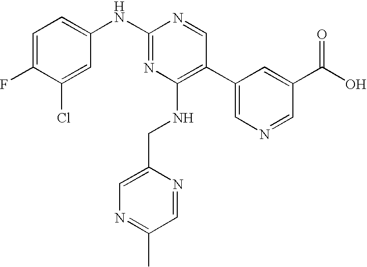 5-[2-[(3-chloro-4- fluorophenyl)amino]-4- [(5-methylpyrazin-2- yl)methylamino]pyrimidin- 5-yl]pyridine-3- carboxylic acid | MS(ES): 466 (M + 1) for C₂₂H₁₇ClFN₇O₂. 400 MHz DMSO-d₆): δ 2.43 (s, 3H), 4.67 (d, J = 5.76 Hz, 2H), 7.20 (t, J = 9.12 Hz, 1H), 7.51 (dt, J = 8.47, 4.04 Hz, 1H), 7.56 (t, J = 5.72 Hz, 1H), 7.92 (s, 1H), 7.96 (dd, J = 6.54, 2.44 Hz, 1H), 8.29 (t, J= 2.00 Hz, 1H), 8.48 (d, J = 5.80 Hz, 1H), 8.84 (d, J = 2.04 Hz, 1H), 9.05 (d, J = 1.88 Hz, 1H), 9.44 (s, 1H), 13.5 (br s, 1H). | ethyl 5-[2- [(3-chloro-4- fluorophenyl) amino]-4-[(5- methylpiperazin- 2- yl)methyl- amino]- pyrimidin- 5- yl]pyridine- 3-carboxylate (Example 115) |
| 243 | 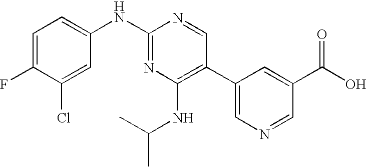 5-[2-[(3-chloro-4- fluorophenyl)amino]-4- (propan-2- ylamino)pyrimidin-5- yl]pyridine-3-carboxylic acid | MS(ES): 402 (M + 1) for C₁₉H₁₇ClFN₅O₂ ¹H NMR (400 MHz DMSO- d₆): δ 1.17 (d, J = 6.56 Hz, 6H), 4.32-4.38 (m, 1H), 6.61 (d, J = 7.8 Hz, 1H), 7.3 (t, J = 9.16 Hz, 1H), 7.55-7.59 (m, 1H), 7.82 (s, 1H), 8.17 (t, J = 2.0 Hz, 1H), 8.28 (dd, J = 6.9, 2.48 Hz, 1H), 8.75 (d, J = 1.96 Hz, 1H), 9.02 (d, J = 1.72 Hz, 1H), 9.46 (s, 1H), 13.5 (br s, 1H). | ethyl 5-[2- [(3-chloro-4- fluorophenyl) amino]-4- (propan-2- ylamino)- pyrimidin- 5- yl]pyridine- 3-carboxylate (Example 116) |
| 244 | 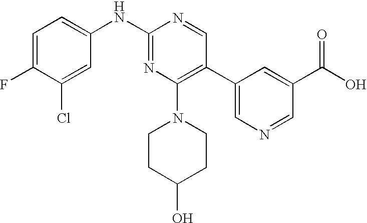 5-[2-[(3-chloro-4- fluorophenyl)amino]-4- (4-hydroxypiperidin-1- yl)pyrimidin-5- yl]pyridine-3-carboxylic acid | MS(ES): 444 (M + 1) for C₂₁H₁₉ClFN₅O₃ ¹H NMR (400 MHz DMSO- d₆): δ 1.31-1.33 (m, 2H), 1.66- 1.69 (m, 2H), 2.94 (t, J = 10.12 Hz, 2H), 3.51-3.53 (m, 2H), 3.63-3.64 (br s, 1H), 4.71 (br s, 1H), 7.33 (t, J = 9.12 Hz, 1H), 7.59-7.61 (m, 1H), 8.10 (s, 1H), 8.21-8.23 (m, 1H), 8.27 (s, 1H), 8.85 (s, 1H), 8.98 (s, 1H), 9.67 (s, 1H), 13.5 (br s, 1H). | ethyl 5-[2- [(3-chloro-4- fluorophenyl) amino]-4-(4- hydroxy- piperidin- 1- yl)pyrimidin- 5- yl]pyridine- 3-carboxylate (Example 117) |
| 245 | 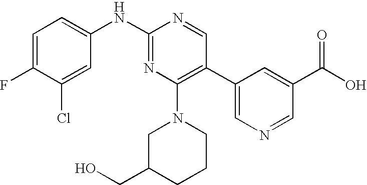 5-[2-[(3-chloro-4- fluorophenyl)amino]-4- [3- (hydroxymethyl)piper- idin-1-yl]pyrimidin-5- yl]pyridine-3-carboxylic acid | MS(ES): 458 (M + 1) for C₂₂H₂₁ClFN₅O₃. ¹H NMR (400 MHz DMSO- d₆): δ 1.12 (m, 1H), 1.37 (m, 1H), 1.53 (m, 2H), 1.66 (m, 1H), 2.56 (t, J = 12.20 Hz, 1H), 2.75 (t, J = 10.64 Hz, 1H), 3.10-3.19 (m, 2H), 3.58 (d, J = 12.44 Hz, 1H), 4.39 (br s, 1H), 7.31 (t, J = 9.04 Hz, 1H), 7.64-7.68 (m, 1H), 8.09 (s, 1H), 8.17 (dd, J = 6.78, 2.44 Hz, 1H), 8.27 (s, 1H), 8.84 (d, J = 1.68 Hz, 1H), 8.97 (s, 1H), 9.65 (s, 1H), 13.5 (br s, 1H). | ethyl 5-[2- [(3-chloro-4- fluorophenyl) amino]-4-[3- (hydroxy- methyl)- piperidin- 1- yl]pyrimidin- 5- yl]pyridine- 3-carboxylate (Example 118) |
| 246 | 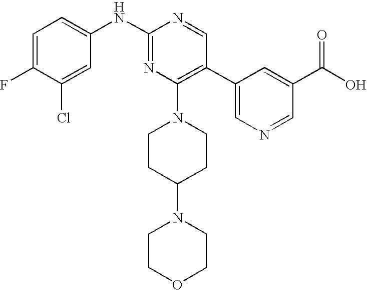 5-[2-[(3-chloro-4- fluorophenyl)amino]-4- (4-morpholin-4- ylpiperidin-1- yl)pyrimidin-5- yl]pyridine-3-carboxylic acid | MS(ES): 513 (M + 1) for C₂₅H₂₆ClFN₆O_{3.} ¹H NMR (400 MHz DMSO- d₆): δ 1.35-1.40 (m, 2H), 1.68- 1.71 (m, 2H), 2.30-2.40 (m, 1H), 2.40-2.50 (m, 4H), 2.77 (t, J = 11.76 Hz, 2H), 3.54 (br s, 4H), 3.72 (d, J = 12.88 Hz, 2H), 7.33 (t, J = 9.16 Hz, 1H), 7.59-7.63 (m, 1H), 8.13 (s, 1H), 8.21 (dd, J = 6.84, 2.48 Hz, 1H), 8.32 (s, 1H), 8.87 (s, 1H), 8.98 (s, 1H), 9.68 (s, 1H). | ethyl 5-[2- [(3-chloro-4- fluorophenyl) amino]-4-(4- morpholin-4- ylpiperidin-1- yl)pyrimidin- 5- yl]pyridine- 3-carboxylate (Example 119) |
| 247 | 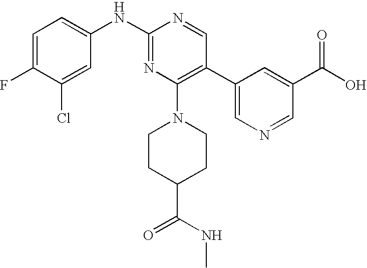 5-[2-[(3-chloro-4- fluoprophenyl)amino]-4- [4- (methylcarbamoyl)- piperidin-1-yl]pyrimidin-5- yl]pyridine-3-carboxylic acid | MS(ES): 485 (M + 1) for C₂₃H₂₂ClFN₆O₃ ¹H NMR (400 MHz, DMSO- d₆): δ 1.46-1.60 (m, 4H), 2.25- 2.30 (m, 1H), 2.52 (d, J = 4.56 Hz, 3H), 2.79 (t, J = 11.32 Hz, 2H), 3.72 (d, J = 13.04 Hz, 2H), 7.33 (t, J = 9.08 Hz, 1H), 7.6 (ddd, J = 9.06, 4.22, 2.68 Hz, 1H), 7.71 (d, J = 4.4 Hz, 1H), 8.1 (s, 1H), 8.21 (dd, J = 6.92, 2.64 Hz, 1H), 8.26 (s, 1H), 8.85 (s, 1H), 8.98 (s, 1H), 9.68 (s, 1H). | ethyl 5-[2- [(3-chloro-4- fluorophenyl) amino]-4-[4- (methylcar- bamoyl)piper- idin-1- yl]pyrimidin- 5- yl]pyridine- 3-carboxylate (Example 120) |
| 248 | 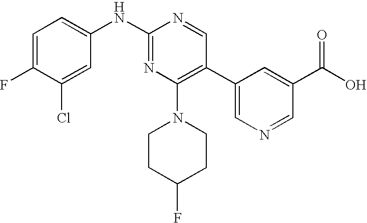 5-[2-[(3-chloro-4- fluorophenyl)amino]-4- (4-fluoropiperidin-1- yl)pyrimidin-5- yl]pyridine-3-carboxylic acid | MS(ES): 446 (M + 1) for C₂₁H₁₈ClF₂N₅O_{2.} ¹H NMR (400 MHz DMSO- d₆): δ 1.62-1.67 (m, 2H), 1.78- 1.89 (m, 2H), 3.16-3.33 (m, 4H), 4.83 (d, J = 48.68 Hz, 1H), 7.33 (t, J = 9.08 Hz, 1H), 7.61 (ddd, J = 9.06, 4.16, 2.76 Hz, 1H), 8.14 (s, 1H), 8.19 (dd, J = 6.88, 2.60 Hz, 1H), 8.31 (t, J = 1.92 Hz, 1H), 8.89 (d, J = 1.96 Hz, 1H), 8.99 (d, J = 1.72 Hz, 1H), 9.72 (s, 1H), 13.7 (br s, 1H). | ethyl 5-[2- [(3-chloro-4- fluorophenyl) amino]-4-(4- fluoropiperidin- 1- yl)pyrimidin- 5- yl]pyridine- 3-carboxylate (Example 121) |
| 249 | 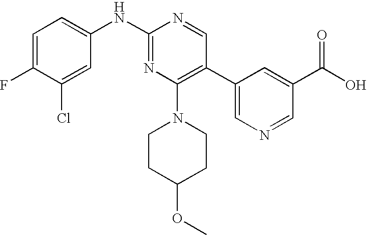 5-[2-[(3-chloro-4- fluorophenyl)amino]-4- (4-methoxypiperidin-1- yl)pyrimidin-5- yl]pyridine-3-carboxylic acid | MS(ES): 458 (M + 1) for C₂₂H₂₁ClFN₅O_{3.} ¹H NMR (400 MHz DMSO- d₆): δ 1.34-1.39 (m, 2H), 1.75- 1.80 (m, 2H), 2.97-3.02 (m, 2H), 3.21 (s, 3H), 3.50-3.60 (m, 2H), 7.33 (t, J = 9.12 Hz, 1H), 7.59-7.63 (m, 1H), 8.11 (s, 1H), 8.2 (dd, J = 6.9, 2.52 Hz, 1H), 8.35 (t, J = 2 Hz, 1H), 8.83 (d, J = 2.12 Hz, 1H), 8.97 (d, J = 1.88 Hz, 1H), 9.67 (s, 1H). | ethyl 5-[2- [(3-chloro-4- fluorophenyl) amino]-4-(4- methoxypip- eridin-1- yl)pyrimidin- 5- yl]pyridine- 3-carboxylate (Example 122) |
| 250 | 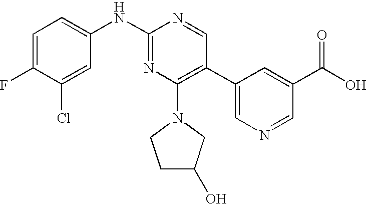 5[2-[(3-chloro-4- fluorophenyl)amino]-4- (3-hydroxypyrrolidin-1- yl)pyrimidin-5- yl]pyridine-3-carboxylic acid | MS(ES): 428 (M) for C₂₀H₁₇ClFN₅O₃. ¹H NMR (400 MHz, DMSO- d₆): δ 1.75 (m, 1H), 1.83-1.85 (m, 1H), 2.8 (br s, 1H), 3.20 (m, 3H), 4.2 (br s, 1H), 4.9 (br s, 1H), 7.32 (t, J = 9.32 Hz, 1H), 7.64-7.66 (m, 1H), 7.95 (s, 1H), 8.10 (br s, 1H), 8.25 d, J = 4.64 Hz, 1H), 8.74 (br s, 1H), 9.0 (s, 1H), 9.56 (s, 1H). | ethyl 5-[2- [(3-chloro-4- fluorophenyl) amino]-4-(3- hydroxy- pyrrolidin-1- yl)pyrimidin- 5- yl]pyridine- 3-carboxylate (Example 123) |
| 251 | 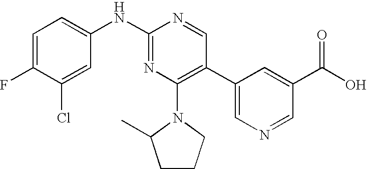 5-[2-[(3-chloro-4- fluorophenyl)amino]-4- (2-methylpyrrolidin-1- yl)pyrimidin-5- yl]pyridine-3-carboxylic acid | MS(ES): 428 (M + 1) for C₂₁H₁₉ClFN₅O₂. ¹H NMR (400 MHz, DMSO- d₆): δ 1.27 (d, J = 6.0 Hz, 3H), 1.48-1.63 (m, 2H), 1.77-1.78 (m, 1H), 2.03-2.08 (m, 1H), 2.66-2.75 (m, 1H), 2.86 (m, 1H), 4.26-4.29 (m, 1H), 7.34 (t, J = 9.16 Hz, 1H), 7.56 (dd, J = 4.64, 2.36 Hz, 1H), 7.99 (s, 1H), 8.14 (s, 1H), 8.24 (d, J = 6.8 Hz, 1H), 8.75 (s, 1H), 8.99 (s, 1H), 9.7 (br s, 1H), 13.7 (br s, 1H). | ethyl 5-[2- [(3-chloro-4- fluorophenyl) amino]-4-(2- methyl- pyrrolidin-1- yl)pyrimidin- 5- yl]pyridine- 3-carboxylate (Example 124) |
| 252 | 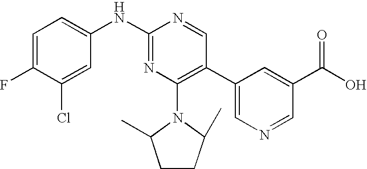 5-[2-[(3-chloro-4- fluorophenyl)amino]-4- (2,5-dimethylpyrrolidin- 1-yl)pyrimidin-5- yl]pyridine-3-carboxylic acid | MS(ES): 440 (M − 1) for C₂₂H₂₁ClFN₅O₂. ¹H NMR (400 MHz DMSO- d₆): δ 0.98 (d, J = 6.28 Hz, 6H), 1.58-1.64 (m, 2H), 1.90- 1.98 (m, 2H), 3.96 (br s, 2H), 7.31 (t, J = 9.12 Hz, 1H), 7.53-7.57 (m, 1H), 7.80 (s, 1H), 8.14 (s, 1H), 8.27 (dd, J = 6.92, 2.48 Hz, 1H),8.69 (br s, 1H), 8.98 (d, J = 1.76 Hz, 1H), 9.49 (s, 1H). | ethyl 5-[2- [(3-chloro-4- fluorophenyl) amino]-4- (2,5- dimethyl- pyrrolidin-1- yl)pyrimidin- 5- yl]pyridine- 3-carboxylate (Example 125) |
| 253 | 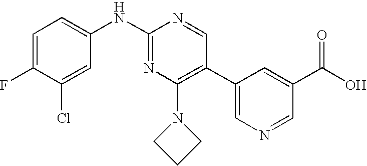 5-[4-(azetidin-1-yl)-2- [(3-chloro-4- fluorophenyl)amino]- pyrimidin-5-yl]pyridine-3- carboxylic acid | MS(ES): 400 (M + 1) for C₁₉H₁₅ClFN₅O_{2.} ¹H NMR (400 MHz DMSO- d₆): δ 2.19 (m, 2H), 3.84 (br s, 4H), 7.42 (t, J = 9.12 Hz, 1H), 7.57 (m, 1H), 7.98 (s, 1H), 8.05 (dd, J = 2.48, 6.72 Hz, 1H), 8.24 (t, J = 1.96 Hz, 1H), 8.81 (d, J = 2.04 Hz, 1H), 9.07 (d, J = 1.85 Hz, 1H), 10.47 (br s, 1H), 13.7 (br s, 1H). | ethyl 5-[4- (azetidin-1- yl)-2-[(3- chloro-4- fluorophenyl) amino]- pyrimidin-5- yl]pyridine- 3-carboxylate (Example 126) |
| 254 | 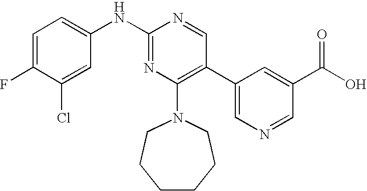 5-[4-(azepan-1-yl)-2-[(3- chloro-4- fluorophenyl)amino]- pyrimidin-5-yl]pyridine-3- carboxylic acid | MS(ES): 442 (M + 1) for C₂₂H₂₁ClFN₅O₂. ¹H NMR (400 MHz, DMSO- d₆): δ 1.41 (br s, 4H), 1.61 (br s, 4H), 3.32 (br s, 4H), 7.32 (t, J = 9.04 Hz, 1H), 7.56 (dt, J = 8.47, 4.24 Hz, 1H), 7.91 (s, 1H), 8.11 (t, J = 2.04 Hz, 1H), 8.26 (dd, J = 6.92, 2.60 Hz, 1H), 8.75 (d, J = 2.16 Hz, 1H), 8.98 (d, J = 1.84 Hz, 1H), 9.56 (s, 1H) | ethyl 5-[4- (azepan-1- yl)-2-[(3- chloro-4- fluorophenyl) amino]- pyrimidin-5- yl]pyridine- 3-carboxylate (Example 127) |
| 255 | 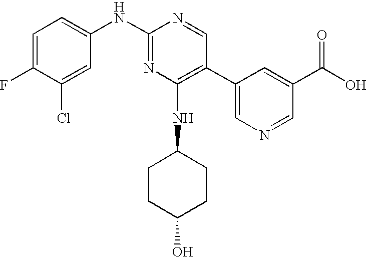 5-[2-[(3-chloro-4- fluorophenyl)amino]-4- [(4- hydroxycyclohexyl)- amino]pyrimidin-5- yl]pyridine-3-carboxylic acid | MS(ES): 458 (M + 1) for C₂₂H₂₁ClFN₅O_{3.} ¹H NMR (400 MHz, DMSO- d₆): δ 1.30 (m, 4H), 1.84 (m, 4H), 3.97 (m, 1H), 4.54 (m, 1H), 6.57 (d, J = 7.96 Hz, 1H), 7.29 (t, J = 8.92 Hz, 1H), 7.55 (d, J = 7.84 Hz, 1H), 7.82 (s, 1H), 8.16 (s, 1H), 8.27 (d, J = 5.76 Hz, 1H), 8.73 (s, 1H), 9.00 (s, 1H), 9.47 (s, 1H), 13.50 (s, 1H). | ethyl 5-[2- [(3-chloro-4- fluorophenyl) amino]-4- [(trans-4- hydroxy- cyclohexyl)- amino] pyrimidin-5- yl]pyridine- 3-carboxylate (Example 128) |
| 256 | 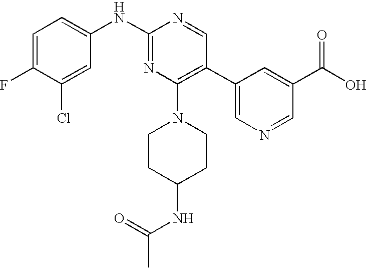 5-[4-(4- acetamidopiperidin-1-yl)- 2-[(3-chloro-4- fluorophenyl)amino]- pyrimidin-5-yl]pyridine-3- carboxylic acid | MS(ES): 485 (M + 1) for C₂₃H₂₂ClFN₆O₃. ¹H NMR (400 MHz, DMSO- d₆): δ 1.22-1.29 (m, 2H), 1.64- 1.67 (m, 2H), 1.78 (s, 3H), 2.90 (t, J = 11.72 Hz, 2H), 3.65 (d, J = 13.28 Hz, 1H), 3.71 (br s, 2H), 7.33 (t, J = 9.04 Hz, 1H), 7.61-7.62 (m, 1H), 7.82 (d, J = 7.56 Hz, 1H), 8.10 (s, 1H), 8.20-8.21 (m, 1H), 8.26 (s, 1H), 8.83 (s, 1H),8.97 (s, 1H), 9.70 (s, 1H). | ethyl 5-[4-(4- acetamido- piperidin-1-yl)- 2-[(3-chloro- 4- fluorophenyl) amino]- pyrimidin-5- yl]pyridine- 3-carboxylate (Example 129) |
| 257 | 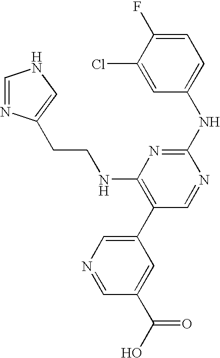 5-(2-[(3-chloro-4- fluorophenyl)amino]-4- {[2-(1H-imidazol-4- yl)ethyl]amino}pyrimidin- 5-yl)pyridine-3- carboxylic acid | MS(ES): 454 (M + 1) for C₂₁H₁₇ClFN₇O₂. ¹H NMR (400 MHz, DMSO- d₆): δ 2.80 (t, J = 7.00 Hz, 2H), 3.59-3.60 (m, 2H), 6.83 (s, 1H), 7.01 (t, J = 5.04 Hz, 1H), 7.25 (t, J = 9.08 Hz, 1H), 7.51 (s, 1H), 7.73 (dt, J = 3.76, 5.96 Hz, 1H), 7.81 (s, 1H), 8.13-8.16 (m, 2H), 8.57 (d, J = 1.88 Hz, 1H), 8.97 (d, J = 1.32 Hz, 1H), 9.43 (s, 1H). | ethyl 5-(2- [(3-chloro-4- fluorophenyl) amino]-4- {[2-(1H- imidazol-4- yl)ethyl]amino}- pyrimidin- 5- yl)pyridine- 3-carboxylate (Example 154) |
| 258 | 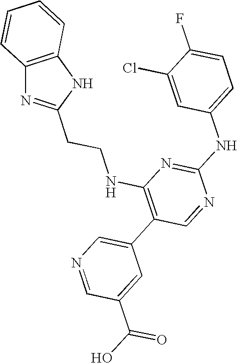 5-(4-{[2-(1H- benzimidazol-2- yl)ethyl]amino}-2-[(3- chloro-4- fluorophenyl)amino]- pyrimidin-5-yl)pyridine-3- carboxylic acid | MS(ES): 504 (M + 1) for C₂₅H₁₉ClFN₇O₂. ¹H NMR (400 MHz, DMSO- d₆): δ 3.14 (t, J = 6.72 Hz, 2H), 3.82-3.84 (m, 2H), 7.11 (dd, J = 2.88, 5.74 Hz, 2H), 7.25 (t, J = 9.24 Hz, 2H), 7.45 (dd, J = 3.20, 5.06 Hz, 2H), 7.73 (dd, J = 8.20,Hz, 1H), 7.89 (s, 1H), 8.15-8.17 (m, 1H), 8.22 (s, 1H), 8.77 (s, 1H), 9.02 (s, 1H), 9.51 (s, 1H), 12.5 (br s, 1H). | ethyl 5-(4- {[2-(1H- benzimidazol- 2- yl)ethyl]amino}- 2-[(3- chloro-4- fluorophenyl) amino]- pyrimidin-5- yl)pyridine- 3-carboxylate (Example 155) |
| 259 | 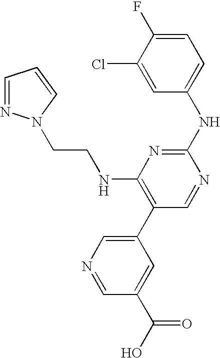 5-(2-[(3-chloro-4- fluorophenyl)amino]-4- {[2-(1H-imidazol-1- yl)ethyl]amino}pyrimidin- 5-yl)pyridine-3- carboxylic acid | MS(ES): 454 (M + 1) for C₂₁H₁₇ClFN₇O₂. ¹H NMR (400 MHz, DMSO- d₆): δ 3.71 (br s, 2H), 4.37 (br s, 2H), 7.02 (br s, 1H), 7.31- 7.37 (m, 2H), 7.55 (br s, 1H), 7.62 (m, 1H), 7.90 (br s, 1H), 8.14 (br s, 1H), 8.65 (br s, 1H), 8.71 (br s, 1H), 9.02 (br s, 1H), 9.52 (br s, 1H). | ethyl 5-(2- [(3-chloro-4- fluorophenyl) amino]-4- {[2-(1H- imidazol-1- yl)ethyl]amino}- pyrimidin- 5- yl)pyridine- 3-carboxylate (Example 130) |
| 260 | 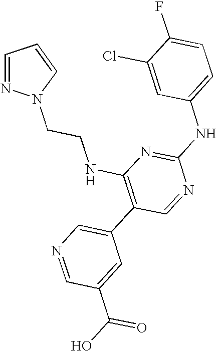 5-(2-[(3-chloro-4- fluorophenyl)amino]-4- {[2-(1H-pyrazol-1- yl)ethyl]amino}pyrimidin- 5-yl)pyridine-3- carboxylic acid | MS(ES): 452 (M − 1) for C₂₁H₁₇ClFN₇O₂. ¹H NMR (400 MHz, DMSO- d₆): δ 3.71 (q, J = 5.80 Hz, 2H), 4.36 (t, J = 6.12 Hz, 2H), 6.22 (t, J = 2.00 Hz,1H), 6.97 (t, J = 5.32 Hz, 1H), 7.31 (t, J = 9.12 Hz, 1H), 7.43 (d, J = 1.64 Hz, 1H), 7.68-7.72(m, 2H), 7.88 (s, 1H), 8.12 (dd, J = 6.86, 2.60 Hz, 1H), 8.16 (t, J = 2.08 Hz, 1H), 8.70 (d, J = 2.16 Hz, 1H), 9.02 (d, J = 1.92 Hz, 1H), 9.50 (s, 1H). | ethyl 5-(2- [(3-chloro-4- fluorophenyl) amino]-4- {[2-(1H- pyrazol-1- yl)ethyl]amino}- pyrimidin- 5- yl)pyridine- 3-carboxylate (Example 131) |
| 261 | 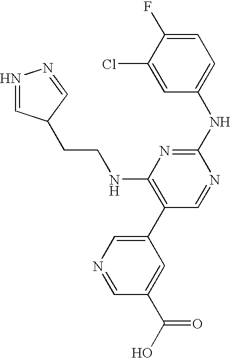 5-(2-[(3-chloro-4- fluorophenyl)amino]-4- {[2-(1H-pyrazol-4- yl)ethyl]amino}pyrimidin- 5-yl)pyridine-3- carboxylic acid | MS(ES): 454 (M + 1) for C₂₁H₁₇ClFN₇O₂. ¹H NMR (400 MHz, DMSO- d₆): δ 2.73 (br s, 2H), 3.59 (br s, 2H), 7.00 (br s, 1H), 7.24 (t, J = 9.16 Hz, 1H), 7.45 (br s, 2H), 7.64 (br s, 1H), 7.85 (s, 1H), 8.18 (s, 2H), 8.72 (br s, 1H), 9.03 (br s, 1H), 9.45 (s, 1H), 13.00 (br s, 1H). | ethyl 5-(2- [(3-chloro-4- fluorophenyl) amino]-4- {[2-(1H- pyrazol-4- yl)ethyl]amino}- pyrimidin- 5- yl)pyridine- 3-carboxylate (Example 156) |
| 262 | 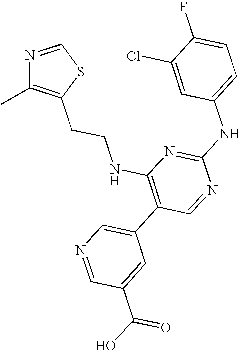 5-(2-[(3-chloro-4- fluorophenyl)amino]-4- {[2-(4-methyl-1,3- thiazol-5- yl)ethyl]amino}pyrimidin- 5-yl)pyridine-3- carboxylic acid | MS(ES): 485 (M + 1) for C₂₂H₁₈ClFN₆O₂S. ¹H NMR (400 MHz, DMSO- d₆): δ 2.25 (s, 3H), 3.06 (t, J = 6.92 Hz, 2H), 3.56-3.57 (m, 2H), 7.05 (t, J = 5.52 Hz, 1H), 7.29 (t, J = 9.12 Hz, 1H), 7.65 (td, J = 3.88, 8.67 Hz, 1H), 7.88 (s, 1H), 8.14 (dd, J = 2.48, 6.84 Hz, 1H), 8.18 (t, J = 1.92 Hz, 1H), 8.73 (d, J = 2.00 Hz, 1H), 8.79 (s, 1H), 9.03 (d, J = 1.64 Hz, 1H), 9.49 (s, 1H), 13.50 (br s, 1H). | ethyl 5-(2- [(3-chloro-4- fluorophenyl) amino]-4- {[2-(4- methyl-1,3- thiazol-5- yl)ethyl]amino}- pyrimidin- 5- yl)pyridine- 3-carboxylate (Example 157) |
| 263 | 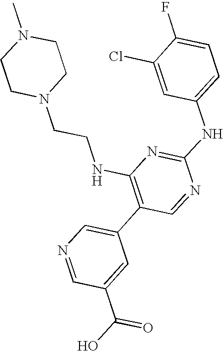 5-(2-[(3-chloro-4- fluorophenyl)amino]-4- {[2-(4-methylpiperazin- 1-yl)ethyl]amino} pyrimidin-5-yl)pyridine- 3-carboxylate acid | MS(ES): 484 (M − 1) and 969 (2M − 1) for C₂₃H₂₅ClFN₇O₂. ¹H NMR (400 MHz, DMSO- d₆): δ 2.39 (s, 3H), 2.50-2.70 (m, 10H), 3.47-3.49 (m, 2H), 6.75 (t, J = 4.56 Hz, 1H), 7.30 (t, J = 9.12 Hz, 1H), 7.65 (ddd, J = 2.76, 4.00, 8.97 Hz, 1H),7.87 (s, 1H), 8.18 (dd, J = 2.52, 6.84 Hz, 1H), 8.22 (d, J = 1.96 Hz, 1H), 8.72 (d, J = 2.12 Hz, 1H), 9.01 (d, J = 1.84 Hz, 1H), 9.47 (s, 1H). | ethyl 5-(2- [(3-chloro-4- fluorophenyl) amino]-4- {[2-(4- methyl- piperazin- 1- yl)ethyl]amino}- pyrimidin- 5- yl)pyridine- 3-carboxylate (Example 132) |
| 264 | 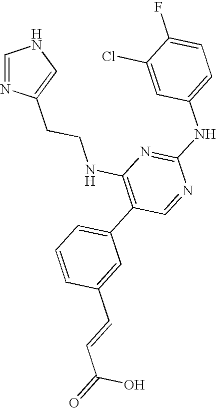 (2E)-3-[3-(2-[(3-chloro- 4-fluorophenyl)amino]-4- {[2-(1H-imidazol-4- yl)ethyl]amino}pyrimidin- 5-yl)phenyl]prop-2- enoic acid | MS(ES): 479 (M + 1) for C₂₄H₂₀ClFn₆O₂. ¹H NMR (400 MHz, DMSO- d₆): δ 2.82 (br s, 2H), 3.63 (br s, 2H), 6.57 (d, J = 15.40 Hz, 1H), 6.81-6.85 (m, 2H), 7.26 (t, J = 9.04 Hz,1H), 7.35 (d, J = 7.04 Hz, 1H), 7.46 (t, J = 7.44 Hz, 1H), 7.53-7.56 (m, 2H), 7.61-7.63 (m, 2H), 7.71 (br s, 1H), 7.82 (s, 1H), 8.18 (d, J = 5.12 Hz, 1H), 9.40 (s, 1H). | ethyl (2E)-3- [3-(2-[(3- chloro-4- fluorophenyl) amino]-4- {[2-(1H- imidazol-4- yl)ethyl]amino}- pyrimidin- 5- yl)phenyl]- prop-2-enoate) (Example 183) |
| 265 | 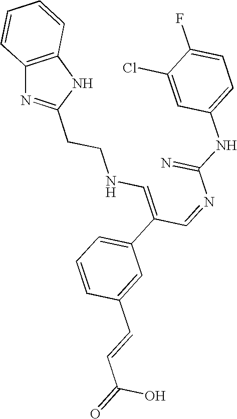 (2E)-3-[3-(4-{[2-(1H- benzimidazol-2- yl)ethyl]amino}-2-[(3- chloro-4-fluorophenyl) amino]pyrimidin-5- yl)phenyl]prop-2-enoic acid | MS(ES): 529 (M + 1) for C₂₈H₂₂ClFN₆O₂. ¹H NMR (400 MHz, DMSO- d₆): δ 3.16-3.19 (m, 2H), 3.85- 3.87 (m, 2H),6.57 (d, J = 16.04 Hz, 1H), 6.99 (t, J = Hz, 1H), 7.13-7.15 (m, 2H), 7.25 (t, J = 9.04 Hz, 1H), 7.35-7.42 (m, 2H), 7.46-7.49 (m, 2H), 7.59 (d, J = 15.96 Hz, 1H), 7.66-7.72 (m, 3H), 7.85 (s, 1H), 8.17 (dd, J = 2.24, 6.68 Hz, 1H), 9.44 (s, 1H), 12.44 (br s, 1H). | ethyl (2E)-3- [3-(4-{[2- (1H- benzimidazol- 2- yl)ethyl]amino}- 2-[(3- chloro-4- fluorophenyl) amino]- pyrimidin-5- yl)phenyl]prop- 2-enoate (Example 184) |
| 266 | 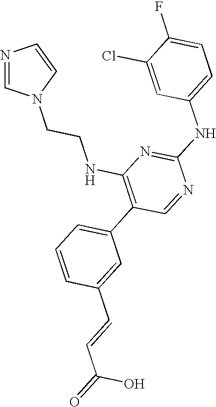 (2E)-3-[3-(2-[(3-chloro- 4-fluorophenyl)amino]-4- {[2-(1H-imidazol-1- yl)ethyl]amino}pyrimidin- 5-yl)phenyl]prop-2- enoic acid | MS(ES): 479 (M + 1) for C₂₄H₂₀ClFN₆O₂. ¹H NMR (400 MHz, DMSO- d₆): δ 3.67-3.69 (m, 2H), 4.23- 4.26 (m, 2H), 6.58 (d, J = 16.00 Hz, 1H), 6.75 (t, J = 5.44 Hz, 1H), 6.94 (br s, 1H), 7.19 (br s, 1H), 7.28-7.34 (m, 2H), 7.47 (t, J = 7.68 Hz,1H), 7.57-7.70 (m, 5H), 7.84 (s, 1H), 8.17 (dd, J = 2.48, 6.88 Hz, 1H), 9.42 (s, 1H). | ethyl (2E)-3- [3-(2-[(3- chloro-4- fluorophenyl) amino]-4- {[2-(1H- imidazol-1- yl)ethyl]amino}- pyrimidin- 5- yl)phenyl]prop- 2-enoate (Example 196) |
| 267 | 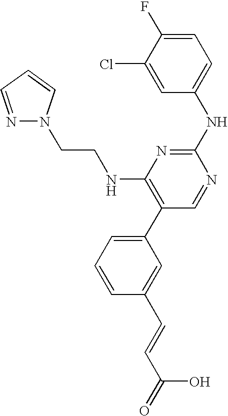 (2E)-3-[3-(2-[(3-chloro- 4-fluorophenyl)amino]-4- {[2-(1H-pyrazol-1- yl)ethyl]amino}pyrimidin- 5-yl)phenyl]prop-2- enoic acid | MS(ES): 477 (M − 1) for C₂₄H₂₀ClFN₆O₂. ¹H NMR (400 MHz, DMSO- d₆): δ 3.75 (q, J = 5.72 Hz, 2H), 4.38 (t, J = 6.12 Hz, 2H), 6.26 (t, J = 1.84 Hz, 1H), 6.56 (d, J = 16.00 Hz, 1H), 6.67 (t, J = 5.24 Hz, 1H), 7.28 (m, 1H), 7.33 (m, 1H), 7.45 (m, 2H), 7.57 (s, 1H), 7.61 (d, J = 16.04 Hz, 1H),7.68 (d, J = 13.60 Hz, 1H), 7.72 (d, J = 2.00 Hz, 1H), 7.85 (s, 1H), 8.14 (dd, J = 6.80, 2.60 Hz, 1H), 9.44 (s, 1H). | ethyl (2E)-3- [3-(2-[(3- chloro-4- fluorophenyl) amino]-4- {[2-(1H- pyrazol-1- yl)ethyl]amino}- pyrimidin- 5- yl)phenyl]prop- 2-enoate (Example 197) |
| 268 | 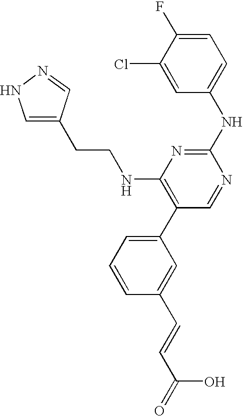 (2E)-3-[3-(2-[(3-chloro- 4-fluorophenyl)amino]-4- {[2-(1H-pyrazol-4- yl)ethyl]amino}pyrimidin- 5-yl)phenyl]prop-2- enoic acid | MS(ES): 479 (M + 1) for C₂₄H₂₀ClFN₆O₂ ¹H NMR (400 MHz, DMSO- d₆): δ 2.74 (t, J = 7.36 Hz, 2H), 3.54-3.59 (m, 2H), 6.57 (d, J = 16.04 Hz, 1H), 6.66- 6.68 (m, 1H), 7.23 (t, J = 9.12 Hz, 1H), 7.35 (d, J = 7.44 Hz, 1H), 7.42 (s, 1H), 7.46 (t, J = 7.56 Hz, 1H), 7.58-7.68 (m, 4H), 7.81 (s, 1H), 8.18 (dd, J = 3.72, 7.58 Hz, 1H), 9.38 (br s, 1H), 12.50 (br s, 1H). | ethyl (2E)-3- [3-(2-[(3- chloro-4- fluorophenyl) amino]-4- {[2-(1H- pyrazol-4- yl)ethyl]amino}- pyrimidin- 5- yl)phenyl]prop- 2-enoate (Example 185) |
| 269 | 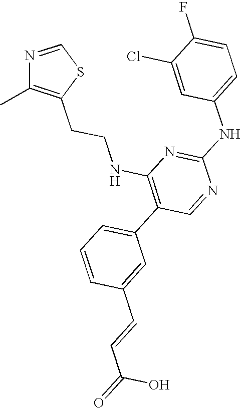 (2E)-3-[3-(2-[(3-chloro- 4-fluorophenyl)amino]-4- {[2-(4-methyl-1,3- thiazol-5- yl)ethyl]amino}pyrimidin- 5-yl)phenyl]prop-2- enoic acid | MS(ES): 510 (M + 1) for C₂₅H₂₁ClFN₅O₂S. ¹H NMR (400 MHz, DMSO- d₆): δ 2.22 (s, 3H), 3.06 (t, J = 6.92 Hz, 2H), 3.57-3.59 (m, 2H), 6.57 (d, J = 16.04 Hz, 1H), 6.84 (br s, 1H), 7.28 (t, J = 9.12 Hz, 1H), 7.37 (d, J = 7.60 Hz, 1H), 7.48 (t, J = 7.32 Hz, 1H), 7.59-7.69 (m, 4H), 7.83 (d, J = 1.08 Hz, 1H), 8.13 (d, J = 5.88 Hz, 1H), 8.79 (d, J = 0.92 Hz, 1H), 9.43 (s, 1H), 12.44 (br s, 1H). | ethyl (2E)-3- [3-(2-[(3- chloro-4- fluorophenyl) amino]-4- {[2-(4- methyl-1,3- thiazol-5- yl)ethyl]amino}- pyrimidin- 5- yl)phenyl]prop- 2-enoate (Example 186) |
| 270 | 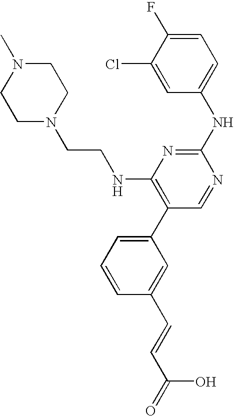 (2E)-3-[3-(2-[(3-chloro- 4-fluorophenyl)amino]-4- {[2-(4-methylpiperazin- 1- yl)ethyl]amino}pyrimidin- 5-yl)phenyl]prop-2- enoic acid | MS(ES): 509 (M − 1) C₂₆H₂₈ClFN₆O₂ ¹H NMR (400 MHz, DMSO- d₆): δ 2.10 (s, 3H), 2.20-2.3 (m, 6H), 3.45-3.47 (m, 2H), 6.46 (t, J = 4.68 Hz, 1H), 6.58 (d, J = 16.00 Hz, 1H), 7.28 (t, J = 9.12 Hz, 1H), 7.41 (d, J = 7.72 Hz, 1H), 7.50 (t, J = 7.68 Hz, 1H), 7.57 (d, J = 16.04 Hz, 1H), 7.65-7.69 (m, 3H), 7.84 (s, 1H), 8.19 (dd, J = 2.52, 6.86 Hz, 1H), 9.41 (s, 1H). | ethyl (2E)-3- [3-(2-[(3- chloro-4- fluorophenyl) amino]-4- {[2-(4- methylpiper- azin-1- yl)ethyl]amino}- pyrimidin- 5- yl)phenyl]prop- 2-enoate (Example 198) |
| 271 | 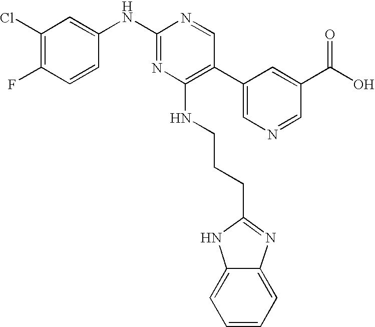 5-(4-(3-(1H- benzo[d]imidazol-2- yl)propylamino)-2-(3- chloro-4- fluorophenylamino)- pyrimidin-5-yl)nicotinic acid | MS(ES): 518 (M + 1) for C₂₆H₂₁ClF₇O₂ ¹H NMR (300 MHz, DMSO- d6) δ ppm 2.09-2.32 (m, 2 H) 3.13 (t, J = 7.44 Hz, 2 H) 3.50 (q, J = 5.59 Hz, 2 H) 7.16- 7.42 (m, 1 H) 7.48 (dd, J = 6.12, 3.11 Hz, 2 H) 7.59- 8.15 (m, 5 H) 8.11-8.36 (m, 1 H) 8.63-8.85 (m, 1 H) 9.02- 9.32 (m, 1 H) 10.24 (s, 1 H) 10.43 (s, 1 H) 14.70 (s, 1 H) | ethyl 5-(4-(3- (1H- benzo[d]- imidazol-2- yl)propyl- amino)-2-(3- chloro-4- fluorophenyl amino)- pyrimidin-5- yl)nicotinate (Example 140) |
| 272 | 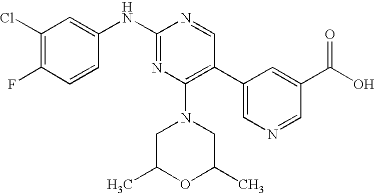 5-(2-(3-chloro-4- fluorophenylamino)-4- (2,6- dimethylmorpholino)- pyrimidin-5-yl)nicotinic acid | MS(ES): 458 (M + 1) for C₂₂H₂₁ClFN₅O₃ ¹H NMR (300 MHz, DMSO- d6) δ ppm 0.96 (d, J = 6.03 Hz, 6 H) 2.37-2.60 (m, 2 H) 3.40- 3.75 (m, 4 H) 7.33 (t, J = 9.14 Hz, 1 H) 7.54 (d, J = 11.68 Hz, 1 H) 8.03-8.39 (m, 3 H) 8.88 (d, J = 2.07 Hz, 1 H) 8.99 (d, J = 1.88 Hz, 1 H) 9.73 (s, 1 H) 13.55 (s, 1 H) | ethyl 5-(2-(3- chloro-4- fluorophenyl amino)-4- (2,6- dimethyl- morpholino)- pyrimidin-5- yl)nicotinate (Example 193) |
| 273 | 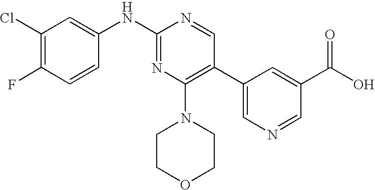 5-(2-(3-chloro-4- fluorophenylamino)-4- morpholinopyrimidin-5- yl)nicotinic acid | MS(ES): 430 (M + 1) for C₂₀H₁₇ClFN₅O₃ ¹H NMR (300 MHz, DMSO- d6) δ ppm 3.11-3.27 (m, 4 H) 3.42-3.71 (m, 4 H) 7.33 (t, J = 9.23 Hz, 1 H) 7.49-7.78 (m, 1 H) 8.02-8.23 (m, 2 H) 8.34 (t, J = 2.07 Hz, 1 H) 8.90 (d, J = 2.07 Hz, 1 H) 8.99 (d, J = 1.88 Hz, 1 H) 9.72 (s, 1 H) | 5-[2-(3- Chloro-4- fluoro- phenylamino)- 4-(2- methoxy- ethylamino)- pyrimidin-5- yl]-nicotinic acid ethyl ester (Example 107) |
| 274 | 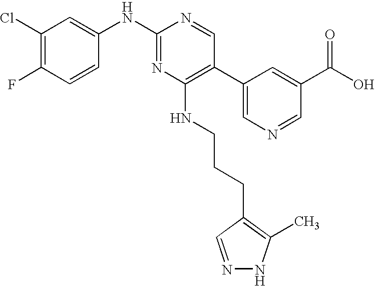 5-(2-(3-chloro-4- fluorophenylamino)-4-(3- (5-methyl-1H-pyrazol-4- yl)propylamino)pyrimidin- 5-yl)nicotinic acid | MS(ES): 482 (M + 1) for C₂₃H₂₁ClFN₇O₂ ¹H NMR (300 MHz, DMSO- d6) δ ppm 1.63-1.94 (m, 2 H) 2.07 (s, 3 H) 2.30-2.46 (m, 2 H) 3.30-3.56 (m, 2 H) 6.87 (t, J = 4.14 Hz, 1 H) 7.16- 7.43 (m, 2 H) 7.48-7.71 (m, 1 H) 7.79 (s, 1 H) 8.10 (s, 1 H) 8.27 (dd, J = 6.88, 2.54 Hz, 1 H) 8.56 (s, 1H) 8.95 (d, J = 1.88 Hz, 1 H) 9.43 (s, 1 H) | ethyl 5-(2-(3- choloro-4- fluorophenyl amino)-4-(3- (5-methyl- 1H-pyrazol- 4- yl)propylamino)- pyrimidin- 5- yl)nicotinate (Example 153) |
| 275 | 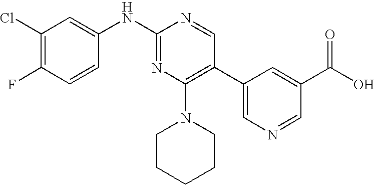 5-(2-(3-chloro-4- fluorophenylamino)-4- (piperidin-1- yl)pyrimidin-5- yl)nicotinic acid | MS(ES): 428 (M + 1) for C₂₁H₁₉ClFN₅O₂ ¹H NMR (300 MHz, DMSO- d6) δ ppm 1.32-1.68 (m, 6 H) 2.99-3.28 (m, 4 H) 7.32 (t, J = 9.04 Hz, 1 H) 7.49-7.81 (m, 1 H) 8.10 (s, 1 H) 8.22 (dd, J = 6.88, 2.54 Hz, 1 H) 8.30 (t, J = 2.07 Hz, 1 H) 8.87 (d, J = 2.26 Hz, 1 H) 8.98 (d, J = 1.88 Hz, 1 H) 9.67 (s, 1 H) 13.52 (s, 1 H) | ethyl 5-(2-(3- chloro-4- fluorophenyl amino)-4- (piperidin-1- yl)pyrimidin- 5- yl)nicotinate (Example 152) |
| 276 | 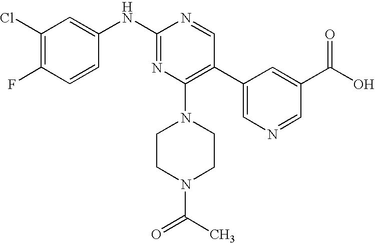 5-(4-(4-acetylpiperazin- 1-yl)-2-(3-chloro-4- fluorophenylamino)- pyrimidin-5-yl)nicotinic acid | MS(ES): 471 (M + 1) for C₂₂H2₀ClFN₆O₃ ¹H NMR (300 MHz, DMSO- d6) δ ppm 1.95 (s, 3 H) 3.05- 3.81 (m, 8 H) 7.34 (t, J = 9.14 Hz, 1 H) 7.64 (d, J = 9.23 Hz, 1 H) 7.95-8.24 (m, 2 H) 8.32 (s, 1 H) 8.89 (d, J = 1.88 Hz, 1 H) 8.92-9.14 (m, 1 H) 9.73 (s, 1 H) 13.51 (s, 1 H) | ethyl 5-(4-(4- acetylpiper- azin-1-yl)-2-(3- chloro-4- fluorophenyl amino)- pyrimidin-5- yl)nicotinate (Example 142) |
| 277 | 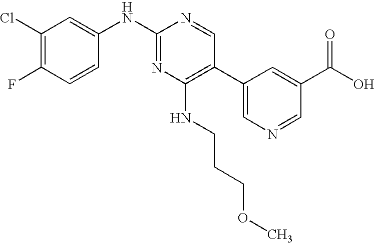 5-(2-(3-chloro-4- fluorophenylamino)-4-(3- methoxypropylamino)- pyrimidin-5-yl)nicotinic acid | MS(ES): 432 (M + 1) for C₂₀H₁₉ClFN₅O₃ ¹H NMR (300 MHz, DMSO- d6) δ 1.56-1.97 (m, 2 H) 3.18 (s, 3 H) 3.28-3.47 (m, 4 H) 7.40 (t, J = 9.04 Hz, 1 H) 7.47-7.67 (m, 1 H) 7.68- 7.97 (m, 2 H) 8.08 (dd, J = 6.78, 2.45 Hz, 1 H) 8.23 (t, J = 1.98 Hz, 1 H) 8.77 (d, J = 2.07 Hz, 1 H) 9.09 (d, J= 1.88 Hz, 1 H) 10.24 (s, 1 H) 13.63 (s, 1 H) | ethyl 5-(2-(3- chloro-4- fluorophenyl amino)-4-(3- methoxy- propylamino)- pyrimidin-5- yl)nicotinate (Example 143) |
| 278 | 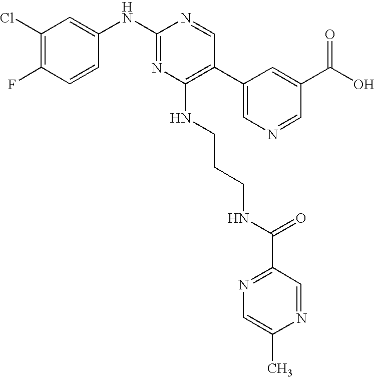 5-(2-(3-chloro-4- fluorophenylamino)-4-(3- (5-methylpieprazine-2- carboxamido)propyl- amino)pyrimidin-5- yl)nicotinic acid | MS(ES): 537 (M + 1) for C₂₅H₂₂ClFN₈O₃ ¹H NMR (300 MHz, DMSO- d6) δ ppm 1.70-2.06 (m, 2 H) 2.56 (s, 3 H) 3.34-3.56 (m, 4 H) 7.03 (s, 1 H) 7.28 (t, J = 9.04 Hz, 1 H) 7.48-7.75 (m, 1 H) 7.85 (s, 1 H) 8.01- 8.38 (m, 2 H) 8.54 (s, 1 H) 8.78 (d, J = 2.07 Hz, 1 H) 8.91 (t, J = 5.84 Hz, 1 H) 8.98 (d, J = 1.32 Hz, 1 H) 9.04 (d, J = 1.88 Hz, 1 H) 9.48 (s, 1 H) 13.43 (s, 1 H) | ethyl 5-(2-(3- chloro-4- fluorophenyl amino)-4-(3- (5- methyl- pyrazine-2- carboxamido) propylamino) pyrimidin-5- yl)nicotinate (Example 141) |
| 279 | 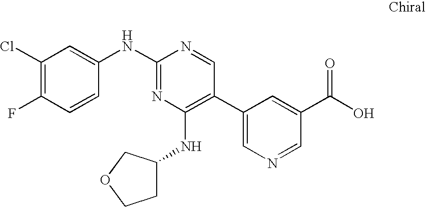 (R)-5-(2-(3-chloro-4- fluorophenylamino)-4- (tetrahydrofuran-3- ylamino)pyrimidin-5- yl)nicotinic acid | MS(ES): 430 (M + 1) for C₂₀H₁₇ClFN₅O₃ ¹H NMR (300 MHz, DMSO- d6) δ ppm 1.71-2.05 (m, 1 H) 2.05-2.34 (m, 1 H) 3.59 (dd, J = 8.76, 4.62 Hz, 1 H) 3.63-3.86 (m, 2 H) 3.94 (dd, J = 8.85, 6.41 Hz, 1 H) 4.44- 4.76 (m, 1 H) 6.94 (d, J = 6.03 Hz, 1 H) 7.32 (t, J = 9.14 Hz, 1 H) 7.49-7.71(m, 1 H) 7.86 (s, 1 H) 8.06-8.39 (m, 2 H) 8.75 (d, J = 2.26 Hz, 1 H) 9.02 (d, J = 1.88 Hz, 1 H) 9.52 (s, 1 H) 13.45 (s, 1 H) | (R)-ethyl-5- (2-(3-chloro- 4- fluorophenyl amino)-4- (tetrahydro- furan-3- ylqamino)- pyrimidin-5- yl)nicotinate (Example 146) |
| 280 | 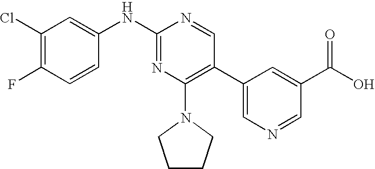 5-(2-(3-chloro-4- fluorophenylamino)-4- (pyrrolidin-1- yl)pyrimidin-5- yl)nicotinic acid | MS(ES): 414 (M + 1) for C₂₀H₁₇ClFN₅O₂ ¹H NMR (300 MHz, DMSO- d6) δ ppm 1.57-1.93 (m, 4 H) 3.00-3.24 (m, 4 H) 7.31 (t, J = 9.14 Hz, 1 H) 7.54-7.77 (m, 1 H) 7.94 (s, 1 H) 8.10 (t, J = 2.07 Hz, 1 H) 8.24 (dd, J = 6.97, 2.45 Hz, 1 H) 8.74 (d, J = 2.07 Hz, 1 H) 8.98 (d, J = 2.07 Hz, 1 H) 9.54 (s, 1 H) 13.50 (s, 1 H) | ethyl 5-(2-(3- chloro-4- fluorophenyl amino)-4- (pyrrolidin-1- yl)pyrimidin- 5- yl)nicotinate (Example 147) |
| 281 | 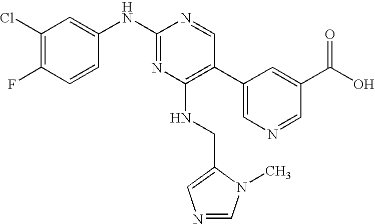 5-(2-(3-chloro-4- fluorophenylamino)-4- ((1-methyl-1H-imidazol- 5- yl)methylamino)pyrimidin- 5-yl)nicotinic acid | MS(ES): 454 (M + 1) for C₂₁H₁₇ClFN₇O₂ ¹H NMR (300 MHz, DMSO- d6) δ ppm 3.14-3.37 (m, 4 H) 3.75-3.98 (m, 4 H) 7.27 (d, J = 3.77 Hz, 1 H) 7.34 (t, J = 9.14 Hz, 1 H) 7.48-7.65 (m, 1 H) 7.69 (d, J = 3.77 Hz, 1 H) 8.10 (dd, J = 6.88, 2.54 Hz, 1 H) 8.21 (s, 1 H) 9.82 (s,1 H) 13.10 (s, 1 H) | ethyl 5-(2-(3- chloro-4- fluorophenyl amino)-4-((1- methyl-1H- imidazol-5- yl)methylamino)- pyrimidin- 5- yl)nicotinate (Example 145) |
| 282 | 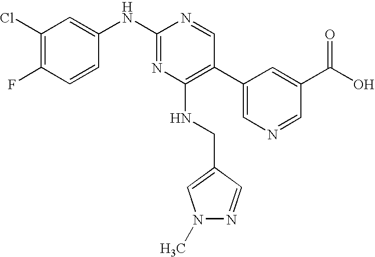 5-(2-(3-chloro-4- fluorophenylamino)-4- ((1-methyl-1H-pyrazol-4- yl)methylamino)pyrimidin- 5-yl)nicotinic acid | MS(ES): 454 (M + 1) for C₂₁H₁₇ClFN₇O₂ ¹H NMR (300 MHz, DMSO- d6) δ ppm 3.73 (s, 3 H) 4.38 (d, J = 5.65 Hz, 2 H) 7.11-7.42 (m, 3 H) 7.54 (s, 1 H) 7.57- 7.75 (m, 1 H) 7.86 (s, 1 H) 7.98-8.35 (m, 2 H) 8.75 (d, J = 2.26 Hz, 1 H) 9.02 (d, J = 1.88 Hz, 1 H) 9.46 (s, 1 H) 13.48 (s,1 H) | ethyl 5-(2-(3- chloro-4- fluorophenyl amino)-4-((1- methyl-1H- pyrazol-4- yl)methyl- amino)- pyrimidin-5- yl)nicotinate (Example 148) |
| 283 | 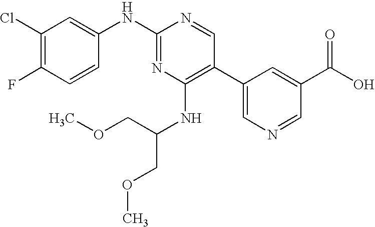 5-(2-(3-chloro-4- fluorophenylamino)-4- (1,3-dimethoxypropan-2- ylamino)pyrimidin-5- yl)nicotinic acid | MS(ES): 462 (M + 1) for C₂₁H₂₁ClFN₅O₄ ¹H NMR (300 MHz, DMSO- d6) δ ppm 3.25 (s, 6 H) 3.36- 3.63 (m, 4 H) 4.42-4.82 (m, 1 H) 6.52 (d, J = 8.67 Hz, 1 H) 7.29 (t, J = 9.14 Hz, 1 H) 7.45- 7.72 (m, 1 H) 7.88 (s, 1 H) 8.03-8.40 (m, 2 H) 8.75 (d, J = 2.26 Hz, 1 H) 9.02 (d, J = 2.07 Hz,1 H) 9.48 (s, 1 H) 13.45 (s, 1 H) | ethyl 5-(2-(3- chloro-4- fluorophenyl amino)-4- (1,3- dimethoxy- propan-2- ylamino)- pyrimidin-5- yl)nicotinate (Example 149) |
| 284 | 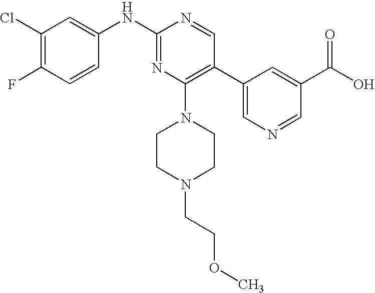 5-(2-(3-chloro-4- fluorophenylamino)-4-(4- (2- methoxyethyl)piperazin- 1-yl)pyrimidin-5- yl)nicotinic acid | MS(ES): 487 (M + 1) for C₂₃H₂₄ClFN₆O₃ ¹H NMR (300 MHz, DMSO- d6) δ ppm 2.30-2.45 (m, 6 H) 3.11-3.27 (m, 7 H) 3.39 (t, J = 5.84 Hz, 2 H) 7.33 (t, J = 9.14 Hz, 1 H) 7.44-7.78 (m, 1 H) 7.96-8.24 (m, 2 H) 8.31 (s, 1 H) 8.87 (d, J = 1.88 Hz, 1 H) 8.98 (d, J = 1.88 Hz, 1 H) 9.71 (s, 1 H) 13.50 (s, 1 H) | ethyl 5-(2-(3- chloro-4- fluorophenyl amino)-4-(4- (2- methoxyethyl)- piperazin-1- yl)pyrimidin- 5- yl)nicotinate (Example 150) |
| 285 | 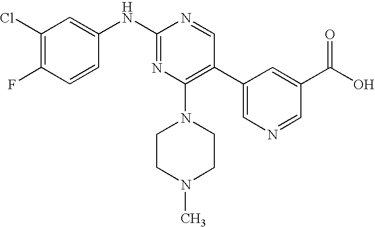 5-(2-(3-chloro-4- fluorophenylamino)-4-(4- methylpiperazin-1- yl)pyrimidin-5- yl)nicotinic acid | MS(ES): 443 (M + 1) for C₂₁H₂₀ClFN₆O₂ ¹H NMR (300 MHz, DMSO- d) δ ppm 2.21 (s, 3 H) 2.31- 2.44 (m, 4 H) 3.15-3.35 (m, 4 H) 7.33 (t, J = 9.04 Hz, 1 H) 7.61 (dd, J = 4.90, 3.20 Hz, 1 H) 8.00-8.25 (m, 2 H) 8.30 (s, 1 H) 8.86 (d, J = 1.32 Hz, 1 H) 8.98 (s, 1 H) 9.72 (s, 1 H) | ethyl 5-(2-(3- chloro-4- fluorophenyl amino)-4-(4- methylpiper- azin-1- yl)pyrimidin- 5- yl)nicotinate (Example 151) |
| 286 | 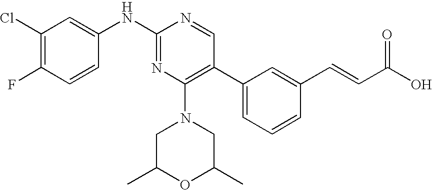 (E)-3-(3-(2-(3-chloro-4- fluorophenylamino)-4- (2,6- dimethylmorpholin-)- pyrimidin-5- yl)phenyl)acrylic acid | MS(ES): 483 (M + 1) for C₂₅H₂₄ClFN₄O₃ ¹H NMR (300 MHz, DMSO- d6) δ ppm 0.94 (d, J = 6.03 Hz, 6 H) 2.18-2.57 (m, 2 H) 3.41- 3.72 (m, 4 H) 6.60 (d, J = 16.01 Hz, 1 H) 7.32 (t, J = 9.04 Hz, 1 H) 7.39-7.72 (m, 5 H) 7.75 (s, 1 H) 8.06 (s, 1 H) 8.23 (dd, J = 6.78, 2.45 Hz, 1H) 9.62 (s, 1 H) 12.44 (s, 1 H) | (E)-ethyl 3- (3-(2-(3- chloro-4- fluorophenyl amino)-4- (2,6- dimethyl- morpholin)- pyrimidin-5- yl)phenyl) acrylate (Example 163) |
| 287 | 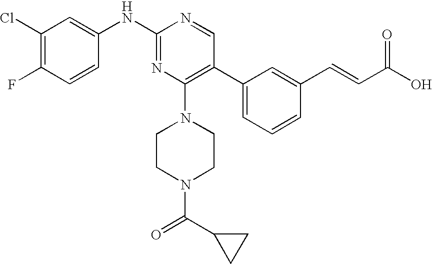 (E)-3-(3-(2-(3-chloro-4- fluorophenylamino)-4-(4- (cyclopropanecarbonyl)- piperazin-1-yl)pyrimidin-5- yl)phenyl)acrylic acid | MS(ES): 522 (M + 1) for C₂₇H₂₅ClFN₅O₃ ¹H NMR (300 MHz, DMSO- d6) δ ppm 0.53-0.83 (m, 4 H) 1.77-2.06 (m, 1 H) 3.16- 3.40 (m, 4 H) 3.41-3.81 (m, 4 H) 6.59 (d, J = 16.01 Hz, 1 H) 7.33 (t, J = 9.14 Hz, 1 H) 7.40- 7.70 (m, 5 H) 7.73 (s, 1 H) 7.99-8.22 (m, 2 H) 9.63 (s, 1 H) | (E)-ethyl 3- (3-(2-(3- chloro-4- fluorophenyl amino)-4-(4- (cyclopropane- carbonyl)- piperazin-1- yl)pyrimidin- 5- yl)phenyl)- acrylate (Example 199) |
| 288 | 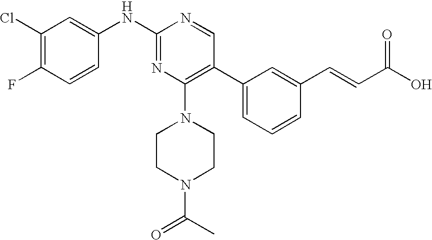 (E)-3-(3-(4-(4- acetylpiperazin-1-yl)-2- (3-chloro-4- fluorophenylamino)- pyrimidin-5- yl)phenyl)acrylic acid | MS(ES): 496 (M + 1) for C₂₅H₂₃ClFN₅O₃ ¹H NMR (300 MHz, DMSO- d6) δ ppm 1.94 (s, 3 H) 3.03- 3.67 (m, 8 H) 6.56 (d, J = 16.01 Hz, 1 H) 7.10-7.79 (m, 7 H) 7.91-8.24 (m, 2 H) 9.62 (s, 1 H) | (E)-ethyl 3- (3-(4-(4- acetylpiper- azin-1-yl)-2-(3- chloro-4- fluorophenyl amino)- pyrimidin-5- yl)phenyl) acrylate Example 162) |
| 289 | 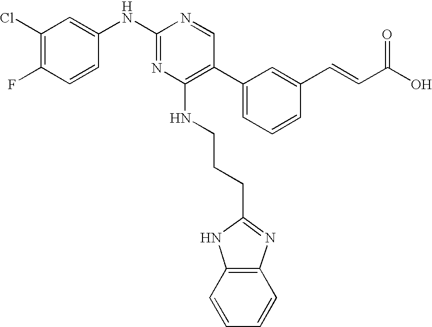 (E)-3-(3-(4-(3-(1H- benzo[d]imidazol-2- yl)propylamino)-2-(3- chloro-4- fluorophenylamino)- pyrimidin-5- yl)phenyl)acrylic acid | MS(ES): 543 (M + 1) for C₂₉H₂₄ClFN₆O₂ ¹H NMR (300 MHz, DMSO- d6) δ ppm 2.06-2.24 (m, 2 H) 3.00-3.14 (m, 2 H) 3.46- 3.60 (m, 2 H) 6.59 (d, J = 16.01 Hz, 1 H) 6.99 (s, 1 H) 7.26 (t, J = 9.14 Hz, 1 H) 7.30-7.45 (m, J = 6.97 Hz, 3 H) 7.48 (t, J = 7.63 Hz, 1 H) 7.52-7.78 (m, 6 H) 7.83 (s, 1 H) 8.20 (dd, J = 6.78, 2.64 Hz, 1 H) 9.51 (s, 1 H) 12.46 (s, 1 H) 14.13 (s, 1 H) | (E)-ethyl 3- (3-(4-(3-(1H- benzo[d]- imidazol-2- yl)propyl- amino)-2-(3- chloro-4- fluorophenyl amino)- pyrimidin-5- yl)phenyl)- acrylate (Example 200) |
| 290 | 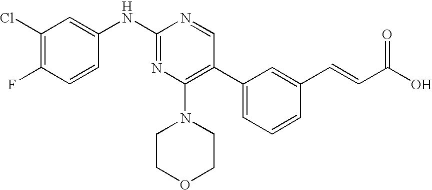 (E)-3-(3-(2-(3-chloro-4- fluorophenylamino)-4- morpholinopyrimidin-5- yl)phenyl)acrylic acid | MS(ES): 455 (M + 1) for C₂₃H₂₀ClFN₄O₃ ¹H NMR (300 MHz, DMSO- d6) δ ppm 3.10-3.24 (m, 4 H) 3.44-3.75 (m, 4 H) 6.60 (d, J = 16.20 Hz, 1 H) 7.32 (t, J = 9.14 Hz, 1 H) 7.41-7.56 (m, 2 H) 7.54-7.71 (m, 3 H) 7.78 (s, 1 H) 7.95-8.31 (m, 2 H) 9.62 (s, 1 H) 12.38 (s, 1 H) | (E)-ethyl 3- (3-(2-(3- chloro-4- fluorophenyl amino)-4- morpholino- pyrimidin-5- yl)phenyl)- acrylate (Example 166) |
| 291 | 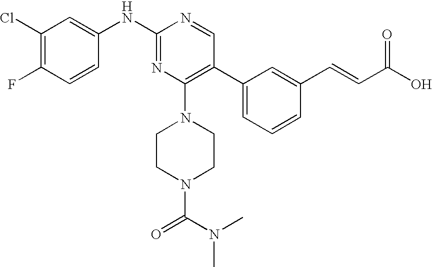 (E)-3-(3-(2-(3-chloro-4- fluorophenylamino)-4-(4- (dimethylcarbamoyl)- piperazin-1-yl)pyrimidin-5- yl)phenyl)acrylic acid | MS(ES): 525 (M + 1) for C₂₆H₂₆ClFN₆O₃ ¹H NMR (300 MHz, DMSO- d6) δ ppm 2.70 (s, 6 H) 2.94- 3.17 (m, 4 H) 3.15-3.30 (m, 4 H) 6.60 (d, J = 16.20 Hz, 1 H) 7.33 (t, J = 9.14 Hz, 1 H) 7.39- 7.57 (m, 2 H) 7.54-7.72 (m, 3 H) 7.76 (s, 1 H) 7.93-8.22 (m, 2 H) 9.62 (s, 1 H) 12.43 (s, 1 H) | (E)-ethyl 3- (3-(2-(3- chloro-4- fluorophenyl amino)-4-(4- (dimethyl- carbamoyl)- piperazin-1- yl)pyrimidin- 5- yl)phenyl)- acrylate (Example 164) |
| 292 | 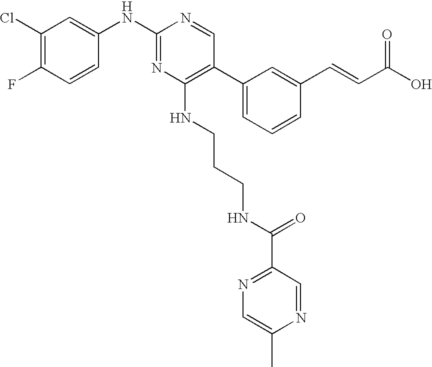 (E)-3-(3-(2-(3-chloro-4- fluorophenylamino)-4-(3- (5-methylpyrazine-2- carboxamido)propylamino)- pyrimidin-5- yl)phenyl)acrylic acid | MS(ES): 562 (M + 1) for C₂₈H₂₅ClFN₇O₃ ¹H NMR (300 MHz, DMSO- d6) δ ppm 1.70-2.04 (m, 2 H) 2.56 (s, 3 H) 3.34-3.62 (m, 4 H) 6.59 (d, J = 16.01 Hz, 1 H) 6.83 (t, J = 4.90 Hz, 1 H) 7.27 (t, J = 9.14 Hz, 1 H) 7.39- 7.56 (m, 2 H) 7.55-7.75 (m, 4 H) 7.81 (s, 1 H) 8.23 (dd, J = 6.97, 2.45 Hz, 1 H) 8.54 (d, J = 0.94 Hz, 1 H) 8.78-9.15 (m, 2 H) 9.40 (s, 1 H) 12.37 (s, 1 H) | (E)-ethyl 3- (3-(2-(3- chloro-4- fluorophenyl amino)-4-(3- (5- methyl- pyrazine-2- carboxamido) propylamino) pyrimidin-5- yl)phenyl)- acrylate (Example 165) |
| 293 | 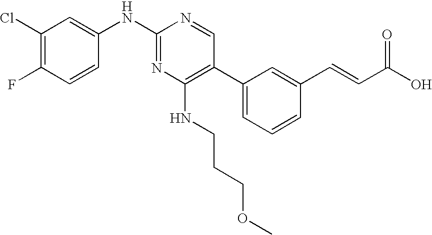 (E)-3-(3-(2-(3-chloro-4- fluorophenylamino)-4-(3- methoxypropylamino)- pyrimidin-5- yl)phenyl)acrylic acid | MS(ES): 457 (M + 1) for C₂₃H₂₂ClFN₄O₃ ¹H NMR (300 MHz, DMSO- d6) δ ppm 1.67-1.96 (m, 2 H) 3.16 (s, 3 H) 3.35-3.58 (m, 4 H) 6.58 (d, J = 16.20 Hz, 1 H) 6.64-6.89 (m, 1 H) 7.29 (t, J = 9.04 Hz, 1 H) 7.36-7.58 (m, 2 H) 7.57-7.76 (m, 4 H) 7.80 (s, 1 H) 8.06-8.40 (m, 1 H) 9.42 (s, 1 H)12.42 (s, 1 H) | (E)-ethyl 3- (3-(2-(3- chloro-4- fluorophenyl amino)-4-(3- methoxy- propyl- amino)- pyrimidin-5- yl)phenyl)- acrylate (Example 167) |
| 294 | 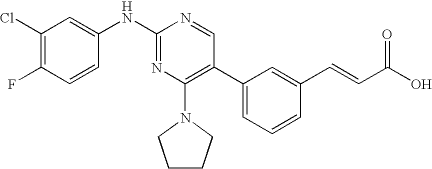 (E)-3-(3-(2-(3-chloro-4- fluorophenylamino)-4- (pyrrolidin-1- yl)pyrimidin-5- yl)phenyl)acrylic acid | MS(ES): 439 (M + 1) for C₂₃H₂₀ClFN₄O₂ ¹H NMR (300 MHz, DMSO- d6) δ ppm 1.63-1.94 (m, 4 H) 3.05-3.23 (m, 4 H) 6.54 (d, J = 16.01 Hz, 1 H) 7.14- 7.76 (m, 8 H) 7.86 (s, 1 H) 8.25 (dd, J = 6.97, 2.64 Hz, 1 H) 9.44 (s, 1 H) | (E)-ethyl 3- (3-(2-(3- chloro-4- fluorophenyl amino)-4- (pyrrolidin-1- yl)pyrimidin- 5- yl)phenyl) acrylate (Example 202) |
| 295 | 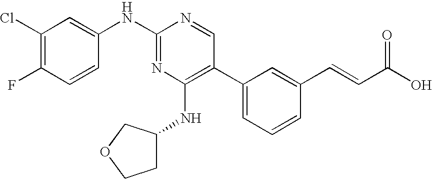 (R,E)-3-(3-(2-(3-chloro- 4-fluorophenylamino)-4- (tetrahydrofuran-3- ylamino)pyrimidin-5- yl)phenyl)acrylic acid | MS(ES): 455 (M + 1) for C₂₃H₂₀ClFN₄O₃ ¹H NMR (300 MHz, DMSO- d6) δ ppm 3.14-3.37 (m, 4 H) 3.75-3.98 (m, 4 H) 7.27 (d, J = 3.77 Hz, 1 H) 7.34 (t, J = 9.14 Hz, 1 H) 7.48-7.65 (m, 1 H) 7.69 (d, J = 3.77 Hz, 1 H) 8.10 (dd, J = 6.88, 2.54 Hz, 1 H) 8.21 (s, 1 H) 9.82 (s,1 H) 13.10 (s, 1 H) | (R,E)-ethyl 3-(3-(2-(3- chloro-4- fluorophenyl amino)-4- (tetrahydro- furan-3- ylamino)- pyrimidin-5- yl)phenyl)- acrylate (Example 169) |
| 296 | 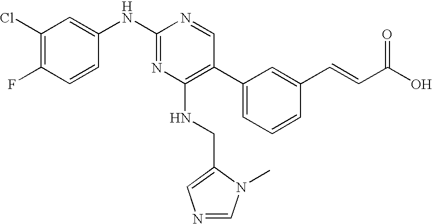 (E)-3-(3-(2-(3-chloro-4- fluorophenylamino)-4- ((1-methyl-1H-imidazol- 5- yl)methylamino)pyrimidin- 5-yl)phenyl)acrylic acid | MS(ES): 479 (M + 1) for C₂₄H₂₀ClFN₆O₂ ¹H NMR (300 MHz, DMSO- d6) δ ppm 3.44-3.67 (s, 3 H) 4.53 (d, J = 5.46 Hz, 2 H) 6.57 (d, J = 16.01 Hz, 1 H) 6.80 (s,1 H) 7.02 (t, J = 5.46 Hz, 1 H) 7.28 (t, J = 9.14 Hz, 1 H) 7.36- 7.45 (m, 1 H) 7.43-7.55 (m, 2 H) 7.53-7.75 (m, 4 H) 7.84 (s, 1 H) 8.12 (dd, J = 6.78, 2.64 Hz, 1 H) 9.38 (s, 1 H) 12.42 (s, 1 H) | (E)-ethyl 3- (3-(2-(3- chloro-4- fluorophenyl amino)-4-((1- methyl-1H- imidazol-5- yl)methyl- amino)- pyrimidin-5- yl)phenyl)- acrylate (Example 172) |
| 297 | 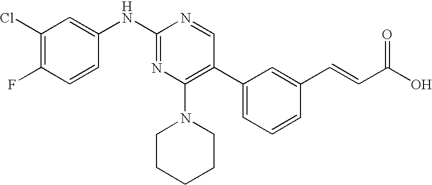 (E)-3-(3-(2-(3-chloro-4- fluorophenylamino)-4- (piperidin-1- yl)pyrimidin-5- yl)phenyl)acrylic acid | MS(ES): 453 (M + 1) for C₂₄H₂₂ClFN₄O₂ ¹H NMR (300 MHz, DMSO- d6) δ ppm 3.14-3.37 (m, 4 H) 3.75-3.98 (m, 4 H) 7.27 (d, J = 3.77 Hz, 1 H) 7.34 (t, J = 9.14 Hz, 1 H) 7.48-7.65 (m, 1 H) 7.69 (d, J = 3.77 Hz, 1 H) 8.10 (dd, J = 6.88, 2.54 Hz, 1 H) 8.21 (s, 1 H) 9.82 (s, 1 H) 13.10 (s, 1H) | (E)-ethyl 3- (3-(2-(3- chloro-4- fluorophenyl amino)-4- (piperidin-1- yl)pyrimidin- 5- yl)phenyl)- acrylate (Example 171) |
| 298 | 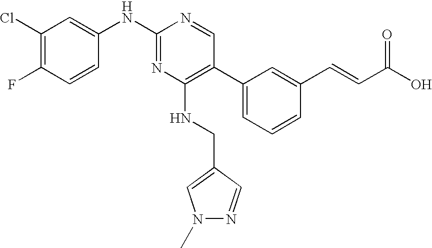 (E)-3-(3-(2-(3-chloro-4- fluorophenylamino)-4- ((1-methyl-1H-pyrazol-4- yl)methylamino)pyrimidin- 5-yl)phenyl)acrylic acid | MS(ES): 479 (M + 1) for C₂₄H₂₀ClFN₆O₂ ¹H NMR (300 MHz, DMSO- d6) δ ppm 3.74 (s, 3 H) 4.39 (d, J = 5.65 Hz, 2 H) 6.43 (d, J = 16.01 Hz, 1 H) 6.85-7.04 (m, 1 H) 7.05-7.58 (m, 8 H) 7.58-7.73 (m, 1 H) 7.80 (s, 1 H) 8.18 (dd, J = 6.88, 2.54 Hz, 1 H) 9.37 (s, 1 H) | (E)-ethyl (3-(2-(3- chloro-4- fluorophenyl amino)-4-((1- methyl-1H- pyrazol-4- yl)methyl- amino)- pyrimidin- 5- yl)phenyl)- acrylate (Example 168) |
| 299 | 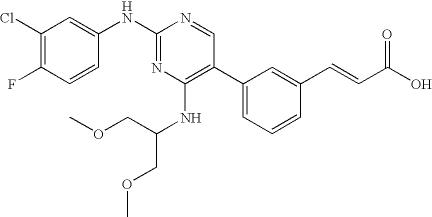 (E)-3-(3-(2-(3-chloro-4- fluorophenylamino)-4- (1,3-dimethoxypropan-2- yl)amino)pyrimidin-5- yl)phenyl)acrylic acid | MS(ES): 487 (M + 1) for C₂₄H₂₄ClFN₄O₄ ¹H NMR (300 MHz, DMSO- d6) δ ppm 3.25 (s, 6 H) 3.36- 3.61 (m, 4 H) 4.32-4.81 (m, 1 H) 6.09 (d, J = 8.48 Hz, 1 H) 6.57 (d, J = 16.01 Hz, 1 H) 7.28 (t, J = 9.14 Hz, 1 H) 7.37-7.48 (m, 1 H) 7.51 (t, J = 7.54 Hz, 1 H) 7.59-7.75 (m, 4H) 7.88 (s, 1 H) 8.15 (dd, J = 6.69, 2.54 Hz, 1 H) 9.43 (s, 1 H) 12.44 (s, 1 H) | (E)-ethyl 3- (3-(2-(3- chloro-4- fluorophenyl amino)-4- (1,3- dimethoxy- propan-2- yl)amino)- pyrimidin-5- yl)phenyl)- acrylate (Example 173) |
| 300 | 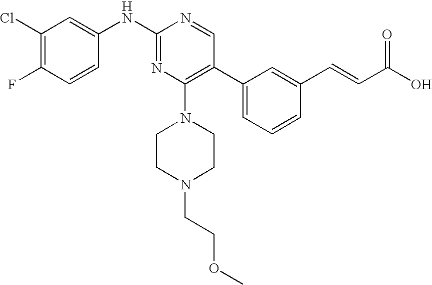 (E)-3-(3-(2-(3-chloro-4- fluorophenylamino)-4-(4- (2- methoxyethyl)piperazin- 1-yl)pyrimidin-5- yl)phenyl)acrylic acid | MS(ES): 512 (M + 1) for C₂₆H₂₇ClFN₅O₃ ¹H NMR (300 MHz, DMSO- d6) δ ppm 2.20-2.48 (m, 6 H) 3.09-3.27 (m, 7 H) 3.38 (t, J = 5.65 Hz, 2 H) 6.60 (d, J = 16.01 Hz, 1 H) 7.32 (t, J = 9.14 Hz, 1 H) 7.43-7.54 (m, 2 H) 7.53-7.70 (m, 3 H) 7.76 (s, 1 H) 8.05 (s, 1 H), 8.17 (dd, J = 6.50, 1.98 Hz, 1 H) 9.61 (s, 1 H) 12.43 (s, 1 H) | (E)-ethyl 3- (3-(2-(3- chloro-4- fluorophenyl amino)-4-(4- (2- methoxyethyl)- piperazin-1- yl)pyrimidin- 5- yl)phenyl)- acrylate (Example 201) |
| 301 | 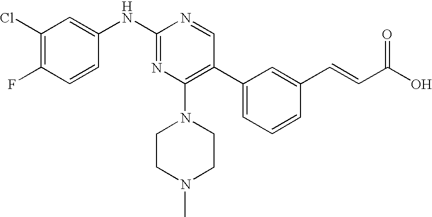 (E)-3-(3-(2-(3-chloro-4- fluorophenylamino)-4-(4- methylpierazin-1- yl)pyrimidin-5- yl)phenyl)acrylic acid | MS(ES): 468 (M + 1) for C₂₄H₂₃ClFN₅O₂ ¹H NMR (300 MHz, DMSO- d6) δ ppm 1.95-2.40 (m, 7 H) 2.98-3.28 (m, 4 H) 6.60 (d, J = 16.01 Hz, 1 H) 7.32 (t, J = 9.14 Hz, 1 H) 7.40-7.55 (m, 2 H) 7.54-7.73 (m, 3 H) 7.76 (s, 1 H) 8.08 (s, 1 H) 8.18 (d, J = 6.22 Hz, 1 H) 9.63 (s, 1 H) 12.44 (s,1 H) | (E)-ethyl 3- (3-(2-(3- chloro-4- fluorophenyl amino)-4-(4- methylpiper- azin-1- yl)pyrimidin- 5- yl)phenyl)- acrylate (Example 170) |
| 302 | 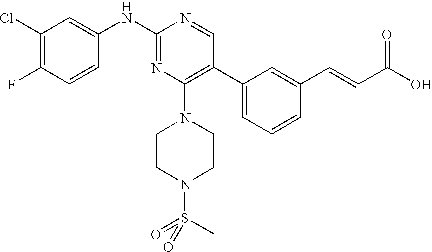 (E)-3-(3-(2-(3-chloro-4- fluorophenylamino)-4-(4- (methylsulfonyl)piperazin- 1-yl)pyrimidin-5- yl)phenyl)acrylic acid | MS(ES): 532 (M + 1) for C₂₄H₂₃ClFN₅O₄S ¹H NMR (300 MHz, DMSO- d6) δ ppm 2.86 (s, 3 H) 2.98- 3.20 (m, 4 H) 3.32-3.49 (m, 4 H) 6.62 (d, J = 16.01 Hz, 1 H) 7.34 (t, J = 9.14 Hz, 1 H) 7.43- 7.58 (m, 2 H) 7.57-7.73 (m, 3 H) 7.79 (s, 1 H) 8.06-8.23 (m, 2 H) 9.67 (s, 1 H) 12.40 (s, 1 H) | (E)-ethyl 3- (3-(2-(3- chloro-4- fluorophenyl amino)-4-(4- (methyl- sulfonyl)- piperazin- 1- yl)pyrimidin- 5- yl)phenyl)- acrylate (Example 174) |
| 303 | 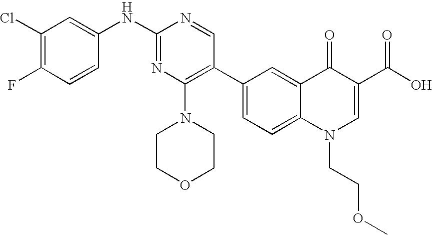 6-(2-(3-chloro-4- fluorophenylamino)-4- morpholinopyrimidin-5- yl)-1-(2-methoxyethyl)- 4-oxo-1,4- dihydroquinone-3- carboxylic acid | MS(ES): 554 (M + 1) for C₂₇H₂₅ClFN₅O₅ ¹H NMR (300 MHz, DMSO- d6) δ 3.11-3.28 (m, 7 H) 3.44-3.65 (m, 4 H) 3.73 (t, J = 4.24 Hz, 2 H) 4.56-5.04 (m, 2 H) 7.34 (t, J = 9.14 Hz, 1 H) 7.52-7.77 (m, 1 H) 7.97- 8.30 (m, 4 H) 8.46 (d, J = 1.70 Hz, 1 H) 8.92 (s, 1H) 9.72 (s, 1 H) 15.18 (s, 1 H) | ethyl 6-(2-(3- chloro-4- fluorophenyl amino)-4- morpholino- pyrimidin-5- yl)-1-(2- methoxyethyl)- 4-oxo-1,4- dihydro- quinoline-3- carboxylate (Example 175) |
| 304 | 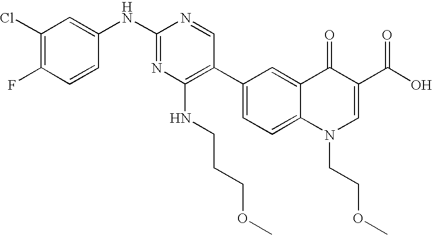 6-(2-(3-chloro-4- fluorophenylamino)-4-(3- methoxypropylamino)- pyrimidin-5-yl)-1-(2- methoxyethyl)-4-oxo- 1,4-dihydroquinoline-3- carboxylic acid | MS(ES): 556 (M + 1) for C₂₇H₂₇ClFN₅O₅ ¹H NMR (300 MHz, DMSO- d6) δ ppm 1.63-1.95 (m, 2 H) 3.18 (s, 3 H) 3.23 (s, 3 H) 3.36-3.52 (m,. 4 H) 3.73 (t, J = 4.43 Hz, 2 H) 4.82 (t, J = 4.05 Hz, 2 H) 7.01 (s, 1 H) 7.31 (t, J = 9.14 Hz, 1 H)7.50- 7.72 (m, 1 H) 7.81-7.90 (m, 1 H) 7.88-8.03 (m, 1 H) 8.06- 8.28 (m, 2 H) 8.35 (d, J = 2.07 Hz, 1 H) 8.94 (s, 1 H) 9.54 (s, 1 H) 15.21 (s, 1 H) | ethyl 6-(2-(3- chloro-4- fluorophenyl amino)-4-(3- methoxypropyl- amino)- pyrimidin-5- yl)-1-(2- methoxyethyl)- 4-oxo-1,4- dihydro- quinoline-3- carboxylate (Example 177) |
| 305 | 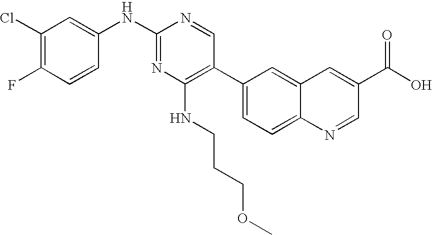 6-(2-(3-chloro-4- fluorophenylamino)-4-(3- methoxypropylamino)- pyrimidin-5-yl)quinoline-3- carboxylic acid | MS(ES): 482 (M + 1) for C₂₄H₂₁ClFN₅O₃ ¹H NMR (300 MHz, DMSO- d6) δ ppm 1.65-1.95 (m, 2 H) 3.15 (s, 3 H) 3.24-3.49 (m, 4 H) 7.40 (t, J = 9.04 Hz, 1 H) 7.51-7.68 (m, 1 H) 7.80- 8.00 (m, 2 H) 8.01-8.34 (m, 3 H) 9.01 (s, 1 H) 9.34 (d, J = 2.07 Hz, 1 H) | methyl 6-(2- (3-chloro-4- fluorophenyl amino)-4-(3- methoxy- propylamino)- pyrimidin-5- yl)quinoline- 3-carboxylate (Example 181) |
| 306 | 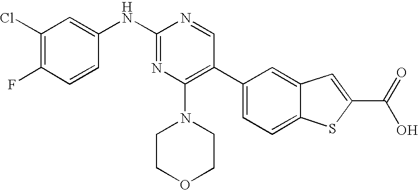 5-(2-(3-chloro-4- fluorophenylamino)-4- morpholinopyrimidin-5- yl)benzo[b]thiophene-2- carboxylic acid | MS(ES): 485 (M + 1) for C₂₃H₁₈ClFN₄O₃S ¹H NMR (300 MHz, DMSO- d6) δ ppm 3.14-3.37 (m, 4 H) 3.75-3.98 (m, 4 H) 7.27 (d, J = 3.77 Hz, 1 H) 7.34 (t, J = 9.14 Hz, 1 H) 7.48-7.65 (m, 1 H) 7.69 (d, J = 3.77 Hz, 1 H) 8.10 (dd, J = 6.88, 2.54 Hz, 1 H) 8.21 (s, 1 H) 9.82 (s, 1 H) 13.10 (s, 1 H) | methyl 5-(2- (3-chloro-4- fluorophenyl amino)-4- morpholino- pyrimidin-5- yl)benzo[b]- thiophene-2- carboxylate (Example 182) |
| 307 | 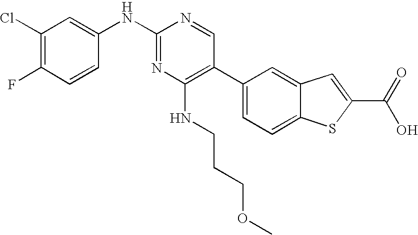 5-(2-(3-choloro-4- fluorophenylamino)-4-(3- methoxypropylamino)- pyrimidin-5- yl)benzo[b]thiophene-2- carboxylic acid | MS(ES): 487 (M + 1) for C₂₃H₂₀ClFN₄O₃S ¹H NMR (300 MHz, DMSO- d6) δ ppm 1.62-1.99 (m, 2 H) 3.14 (s, 3 H) 3.34-3.58 (m, 4 H) 6.65 (t, J = 5.37 Hz, 1 H) 7.10-7.38 (m, 2 H) 7.47 (s, 1 H) 7.57-7.71 (m, 1 H) 7.79 (s, 2 H) 7.88 (d, J = 8.10 Hz, 1 H) 8.24 (dd, J = 6.88, 2.54 Hz, 1 H) 9.35 (s, 1 H) | methyl 5-(2- (3-chloro-4- fluorophenyl amino)-4-(3- methoxypropyl- amino)- pyrimidin-5- yl)benzo[b]- thiophene-2- carboxylate (Example 180) |

The following examples were prepared using the general method described above for Example 214 using 1N sodium hydroxide (1-2 equivalents), dioxane or THF as solvent and the starting material (SM) indicated.

| Ex | Compound | Data | SM |
|---|---|---|---|
| 308 | 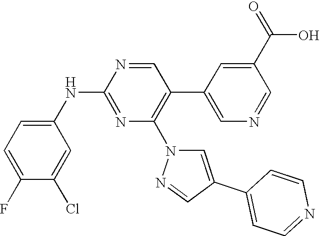 5-{2-[(3-chloro-4- fluorophenyl)amino]- 4-[4-(pyridin-4-yl)- 1H-pyrazol-1- yl]pyrimidin-5- yl}pyridine-3- carboxylic acid | MS(ES): 488 (M + 1) for C₂₄H₁₅ClFN₇O₂. ¹H NMR (400 MHz, DMSO-d₆): δ 7.43 (t, J = 8.92 Hz, 1H), 7.68- 7.72 (m, 3H), 8.09 (s, 1H), 8.13 (d, J = 4.76 Hz, 1H), 8.25 (s, 1H), 8.58-8.72 (m, 4H), 8.98 (s, 1H), 9.06 (s, 1H), 10.38 (s, 1H). | Ethyl 5-{2- [(3-chloro-4- fluorophenyl) amino]-4-[4- (pyridin-4- yl)-1H- pyrazol-1- yl]pyrimidin- 5- yl}pyridine- 3-carboxylate (Example 203) |
| 309 | 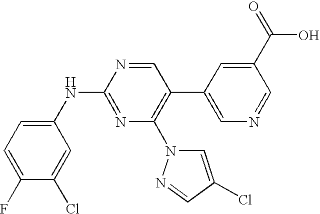 5-{2-[(3-chloro-4- fluorophenyl)amino]- 4-(4-chloro-1H- pyrazol-1- yl)pyrimidin-5- yl}pyridine-3- carboxylic acid | MS(ES): 445 (M + 1) for C₁₉H₁₁C₁₂FN₆O₂. ¹H NMR (400 MHz, DMSO-d₆): δ 7.42 (t, J = 9.08 Hz, 1H), 7.72 (d, J = 2.52 Hz, 1H), 7.74 (s, 1H), 8.02 (d, J = 4.44 Hz, 1H), 8.09 (s, 1H), 8.58 (s, 1H), 8.64 (s, 1H), 8.71 (s, 1H), 8.99 (s, 1H), 10.34 (br s, 1H), 13.50 (br s, 1H). | ethyl 5-{2- [(3-chloro-4- fluorophenyl) amino]-4-(4- chloro-1H- pyrazol-1- yl)pyrimidin- 5- yl}pyridine- 3-carboxylate (Example 135) |
| 310 | 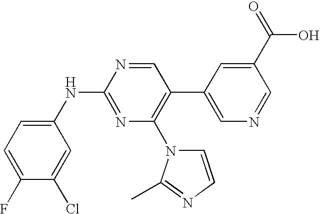 5-{2-[(3-chloro-4- fluorophenyl)amino]- 4-(2-methyl-1H- imidazol-1- yl)pyrimidin-5- yl}pyridine-3- carboxylic acid | MS(ES): 425 (M + 1) for C₂₀H₁₄ClFN₆O₂. ¹H NMR (400 MHz, DMSO-d₆): δ 2.20 (s, 3H), 6.80 (s, 1H), 7.04 (s, 1H), 7.41 (t, J = 8.85 Hz, 1H), 7.66 (m, 1H), 7.98 (s, 1H), 8.06 (d, J = 4.36 Hz, 1H)), 8.52 (s, 1H), 8.90 (s, 1H), 8.97 (s, 1H), 10.42 (s, 1H), 13.6 (br s, 1H). | ethyl 5-{2- [(3-chloro-4- fluorophenyl) amino]-4-(2- methyl-1H- imidazol-1- yl)pyrimidin- 5- yl}pyridine- 3-carboxylate (Example 205) |
| 311 | 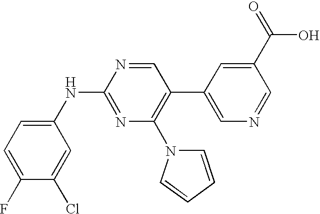 5-{2-[(3-chloro-4- fluorophenyl)amino]- 4-(1H-pyrrol-1- yl)pyrimidin-5- yl}pyridine-3- carboxylic acid | MS(ES): 410 (M + 1) for C₂₀H₁₃ClFN₅O₂ ¹H NMR (400 MHz, DMSO-d₆): δ 6.16 (d, J = 1.92 Hz, 2H), 6.90 (d, J = 1.96 Hz, 2H), 7.40 (t, J = 5.44 Hz, 1H), 7.69-7.73 (m, 1H), 8.00 (s, 1H), 8.11 (dd, J = 2.56, 6.76 Hz, 1H), 8.34 (d, J = 1.64 Hz, 1H), 8.53 (s, 1H), 8.94 (s 1H), 10.19 (s, 1H). | ethyl 5-{2- [(3-chloro-4- fluorophenyl) amino]-4- (1H-pyrrol-1- yl)pyrimidin- 5- yl}pyridine- 3-carboxylate (Example 138) |
| 312 | 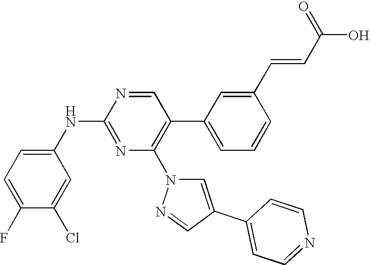 (2E)-3-(3-{2-[(3- chloro-4- fluorophenyl)amino]- 4-[4-(pyridin-4-yl)- 1H-pyrazol-1- yl]pyrimidin-5- yl}phenyl)prop-2- enoic acid | MS(ES): 513 (M + 1) for C₂₇H₁₈ClFN₆O₂. ¹H NMR (400 MHz, DMSO-d₆): δ 6.52 (d, J = 16.60 Hz, 1H), 7.16 (d, J = 1.44 Hz, 1H), 7.18-7.53 (m, 2H), 7.57-7.67 (m, 5H), 7.73- 7.76 (m, 1H), 8.16 (d, J = 4.96 Hz, 1H), 8.26 (s, 1H), 8.57 (d, J = 1.44 Hz, 1H), 8.74 (s, 1H), 8.98 (s, 1H), 10.34 (s, 1H), 12.20 (br s, 1H). | ethyl (2E)-3- (3-{2-[(3- chloro-4- fluorophenyl) amino]-4-[4- (pyridin-4- yl)-1H- pyrazol-1- yl]pyrimidin- 5- yl}phenyl) prop-2-enoate (Example 188) |
| 313 | 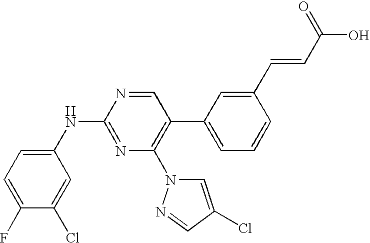 (2E)-3-(3-{2-[(3- chloro-4- fluorophenyl)amino]- 4-(4-chloro-1H- pyrazol-1- yl)pyrimidin-5- yl}phenyl)prop-2- enoic acid | MS(ES): 470 (M + 1) for C₂₂H₁₄Cl₂FN₅O₂. ¹H NMR (400 MHz, DMSO-d₆): δ 6.51 (d, J = 15.96 Hz, 1H), 7.11 (d, J = 7.72 Hz, 1H), 7.33-7.45 (m, 3H), 7.51-7.57 (m, 2H), 7.71- 7.74 (m, 1H), 7.75 (s, 1H), 8.06 (dd, J = 2.40, 6.54 Hz, 1H), 8.46 (s, 1H), 8.71 (s, 1H), 10.31 (br s, 1H). | ethyl (2E)-3- (3-{2-[(3- chloro-4- fluorophenyl) amino]-4-(4- chloro-1H- pyrazol-1- yl)pyrimidin- 5- yl}phenyl) prop-2-enoate (Example 159) |
| 314 | 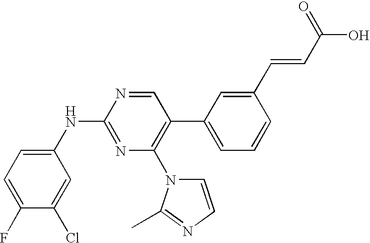 (2E)-3-(3-{2-[(3- chloro-4- fluorophenyl)amino]- 4-(2-methyl-1H- imidazol-1- yl)pyrimidin-5- yl}phenyl)prop-2- enoic acid | MS(ES): 450 (M + 1) for C₂₃H₁₇ClFN₅O₂. ¹H NMR (400 MHz, DMSO-d₆): δ 2.10 (s, 3H), 6.47 (d, J = 16.00 Hz, 1H), 6.82 (br s, 1H), 7.09 (s, 1H), 7.10 (s, 1H), 7.35-7.42 (m, 2H), 7.45 (s, 1H), 7.50 (d, J = 16.00 Hz, 1H), 7.62-7.69 (m, 2H), 8.07-8.08 (m, 1H), 8.86 (s, 1H), 10.35 (s, 1H), 12.47 (br s, 1H). | ethyl (2E)-3- (3-{2-[(3- chloro-4- fluorophenyl) amino]-4-(2- methyl-1H- imidazol-1- yl)pyrimidin- 5- yl}phenyl)- prop-2-enoate (Example 190) |
| 315 | 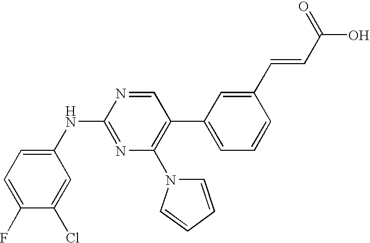 (2E)-3-(3-{2-[(3- chloro-4- fluorophenyl)amino]- 4-(1H-pyrrol-1- yl)pyrimidin-5- yl}phenyl)prop-2- enoic acid | MS(ES): 433 (M − 1) for C₂₃H₁₆ClFN₄O₂. ¹H NMR (400 MHz, DMSO-d₆): δ 6.16 (t, J = 1.92 Hz, 2H), 6.51 (d, J = 16.00 Hz, 1H), 6.92 (t, J = 2.04 Hz, 2H), 7.21 (d, J = 7.56 Hz, 1H), 7.35-7.43 (m, 3H), 7.54 (s, 1H), 7.62 (d, J = 7.92 Hz, 1H), 7.68-7.71 (m, 1H), 8.11 (dd, J = 2.48, 6.74 Hz, 1H), 8.55 (s, 1H), 10.16 (s, 1H). | ethyl (2E)-3- (3-{2-[(3- chloro-4- fluorophenyl) amino]-4- (1H-pyrrol-1- yl)pyrimidin- 5- yl}phenyl)- prop-2-enoate (Example 195) |

### Example 316

### 5-{2-[(3-chloro-4-fluorophenyl)amino]-4-(3,5-dimethyl-1H-pyrazol-1-yl)pyrimidin-5-yl}pyridine-3-carboxylic acid

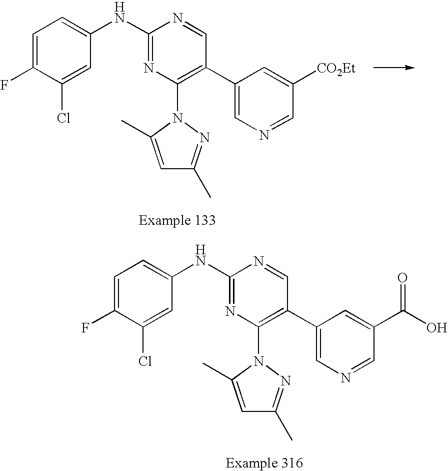

Ethyl 5-{2-[(3-chloro-4-fluorophenyl)amino]-4-(3,5-dimethyl-1H-pyrazol-1-yl)pyrimidin-5-yl}pyridine-3-carboxylate (Example 133, 100 mg, 0.22 mmol) was dissolved in tetrahydrofuran (1 ml) and treated with a suspension of aqueous barium hydroxide (35 mg, 0.88 mmol) in water (1 ml). The mixture was allowed to stir at room temperature for 4 h. After completion of the reaction, the mixture was then carefully acidified with 1 N HCl and the precipitate that formed was filtered, washed with water, and dried to yield the title compound (65 mg).

MS(ES): 439 (M+1) for C₂₁H₁₆ClFN₆O₂.

¹H NMR (400 MHz, DMSO-d₆) δ 1.92 (s, 3H), 2.36 (s, 3H), 6.07 (s, 1H), 7.41 (t, J=9.08 Hz, 1H), 7.62-7.67 (m, 1H), 7.88 (t, J=2.12 Hz, 1H), 8.09 (dd, J=2.48, 6.74 Hz, 1H), 8.37 (d, J=1.80 Hz, 1H), 8.83 (s, 1H), 8.91 (d, J=1.88 Hz, 1H), 10.28 (s, 1H).

The following examples were prepared using the general method described above for Example 316 using barium hydroxide (2-4 equivalents), dioxane or THF and the starting material (SM) indicated.

| Ex | Compound | Data | SM |
|---|---|---|---|
| 317 | 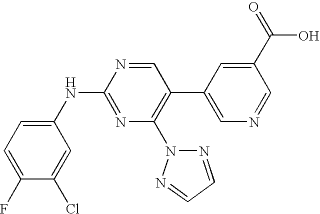 5-{4-(2H-1,2,3-triazol-2- yl)-2-[(3-chloro-4- fluorophenyl)amino] pyrimidin-5-yl}pyridine-3- carboxylic acid | MS(ES): 412 (M + 1) for C₁₈H₁₁ClFN₇O₂. ¹H NMR (400 MHz, DMSO- d₆): δ 7.43 (t, J = 9.20 Hz, 1H), 7.76-7.80 (m, 1H), 7.96 (s, 1H), 8.12 (s, 2H), 8.23 (d, J = 4.40 Hz, 1H), 8.55 (d, J = 1.60 Hz, 1H), 8.87 (s, 1H), 9.00 (br s, 1H), 10.571 (s, 1H). | ethyl 5-{2- [(3-chloro-4- fluorophenyl) amino]-4- (2H-1,2,3- triazol-2- yl)pyrimidin- 5- yl}pyridine- 3-carboxylate (Example 206) |
| 318 | 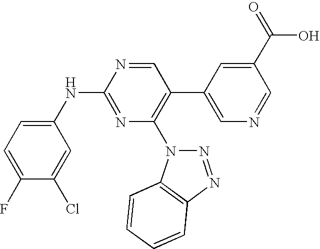 5-{4-(1H-benzotriazol-1- yl)-2-[(3-chloro-4- fluorophenyl)amino] pyrimidin-5-yl}pyridine-3- carboxylic acid | MS(ES): 462 (M + 1) for C₂₂H₁₃ClFN₇O₂. ¹H NMR (400 MHz, DMSO- d₆): δ 7.43 (t, J = 9.08 Hz, 1H), 7.53-7.58 (m, 1H), 7.63- 7.66 (m, 1H), 7.71-7.75 (m, 1H), 8.12-8.16 (m, 3H), 8.32 (d, J = 8.16 Hz, 1H), 8.68 (d, J = 2.16 Hz, 1H), 8.91 (s, 1H), 9.00 (d, J = 1.92 Hz, 1H), 10.47 (s, 1H), 13.50 (br s, 1H). | ethyl 5-{4- (1H- benzotriazol- 1-yl)-2-[(3- chloro-4- fluorophenyl) amino] pyrimidin-5- yl}pyridine- 3-carboxylate (Example 209) |
| 319 | 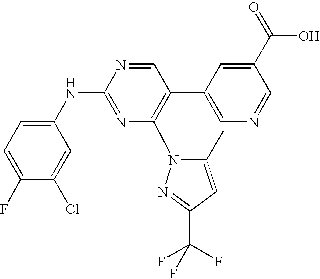 5-{2-[(3-chloro-4- fluorophenyl)amino]-4- [5-methyl-3- (trifluoromethyl)-1H- pyrazol-1-yl]pyrimidin- 5-yl}pyridine-3- carboxylic acid | MS(ES): 493 (M + 1) for C₂₁H₁₃ClF₄N₆O₂. ¹H NMR (400 MHz, DMSO- d₆): δ 2.42 (s, 3H), 6.77 (s, 1H), 7.43 (t, J = 9.08 Hz, 1H), 7.64-7.68 (m, 1H), 7.82 (t, J = 2.04 Hz, 1H),8.06 (d, J = 4.24 Hz, 1H), 8.53 (d, J = 1.92 Hz, 1H), 8.95 (d, J = 1.76 Hz, 1H), 8.99 (s, 1H), 10.48 (s, 1H), 13.40 (br s, 1H). | ethyl 5-{2- [(3-chloro-4- fluorophenyl) amino]-4-[5- methyl-3- (trifluoro- methyl)-1H- pyrazol-1- yl]pyrimidin- 5- yl}pyridine- 3-carboxylate (Example 134) |
| 320 | 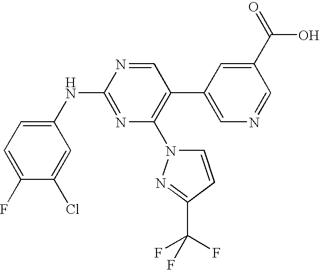 5-{2-[(3-chloro-4- fluorophenyl)amino]-4- [3-(trifluoromethyl)-1H- pyrazol-1-yl]pyrimidin- 5-yl}pyridine-3- carboxylic acid | MS(ES): 479 (M + 1) for C₂₀H₁₁ClF₄N₆O₂. ¹H NMR (400 MHz, DMSO- d₆): δ 7.04 (d, J = 2.60 Hz, 1H), 7.43 (t, J = 9.12 Hz, 1H), 7.73 (ddd, J = 2.64, 4.12, 9.02 Hz, 1H),8.00 (s, 1H), 8.07 (dd, J = 2.96, 6.94 Hz, 1H), 8.53 (s, 1H), 8.59 (s, 1H), 8.82 (s, 1H), 8.99 (s, 1H), 10.46 (s, 1H). | ethyl 5-{2- [(3-chloro-4- fluorophenyl) amino]-4-[3- (trifluoro- methyl)-1H- pyrazol-1- yl]pyrimidin- 5- yl}pyridine- 3-carboxylate (Example 136) |
| 321 | 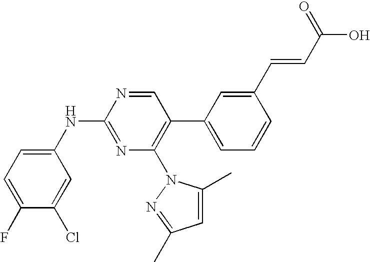 (2E)-3-(3-{2-[(3-chloro- 4-fluorophenyl)amino]-4- (3,5-dimethyl-1H- pyrazol-1-yl)pyrimidin- 5-yl}phenyl)prop-2-enoic acid | MS(ES): 464 (M + 1) for C₂₄H₁₉ClFN₅O₂. ¹H NMR (400 MHz, DMSO- d₆): δ 2.03 (s, 3H), 2.13 (s, 3H), 6.03 (s, 1H), 6.42 (d, J = 16.00 Hz, 1H), 7.04 (d, J = 7.80 Hz, 1H), 7.31-7.35 (m, 2H), 7.40 (t, J = 9.00 Hz, 1H), 7.51 (d, J = 16.12 Hz, 1H), 7.56 (d, J = 7.52 Hz, 1H), 7.66 (m, 1H), 8.11 (dd, J = 2.68, 6.86 Hz, 1H), 8.87 (s, 1H), 10.29 (s, 1H), 12.50 (br s, 1H). | ethyl (2E)-3- (3-{2-[(3- chloro-4- fluorophenyl) amino]-4- (3,5- dimethyl-1H- pyrazol-1- yl)pyrimidin- 5- yl}phenyl) prop-2-enoate (Example 158) |
| 322 | 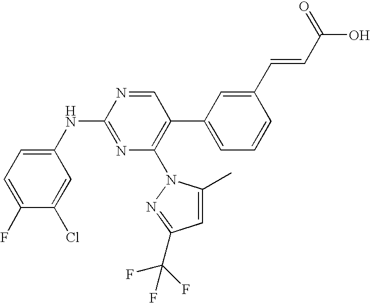 (2E)-3-(3-{2-[(3-chloro- 4-fluorophenyl)amino]-4- [5-methyl-3- (trifluoromethyl)-1H- pyrazol-1-yl]pyrimidin- 5-yl}phenyl)prop-2-enoic acid | MS(ES): 518 (M + 1) for C₂₄H₁₆ClF₄N₅O₂. ¹H NMR (400 MHz, DMSO- d₆): δ 2.22 (s, 3H), 6.44 (d, J = 15.92 Hz, 1H), 6.72 (s, 1H), 7.04 (d, J = 7.60 Hz, 1H), 7.33-7.37 (m, 2H), 7.42 (t, J = 9.12 Hz, 1H), 7.49 (d, J = 15.96 Hz, 1H), 7.61 (d, J = 7.92 Hz, 1H), 7.65-7.67 (m, 1H), 8.08 (d, J = 4.52 Hz, 1H), 8.98 (s, 1H), 12.4 (br s, 1H). | ethyl (2E)-3- (3-{2-[(3- chloro-4- fluorophenyl) amino]-4-[5- methyl-3- (trifluoro- methyl)-1H- pyrazol-1- yl]pyrimidin- 5- yl}phenyl) prop-2-enoate (Example 187) |
| 323 | 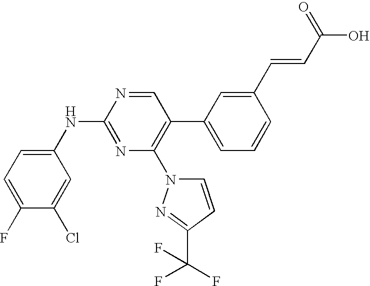 (2E)-3-(3-{2-[(3-chloro- 4-fluorophenyl)amino]-4- [3-(trifluoromethyl)-1H- pyrazol-1-yl]pyrimidin- 5-yl}phenyl)prop-2-enoic acid | MS(ES): 504 (M + 1) for C₂₃H₁₄ClF₄N₅O₂. ¹H NMR (400 MHz, DMSO- d₆): δ 6.48 (d, J = 16.00 Hz, 1H), 7.00 (d, J = 2.36 Hz, 1H), 7.14 (d, J = 7.56 Hz, 1H), 7.35-7.43 (m, 2H), 7.52 (s, 1H), 7.54 (d, J = 15.84 Hz, 1H), 7.64 (d, J = 7.68 Hz, 1H), 7.71-7.73 (m, 1H), 8.12 (dd, J = 2.48, 6.66 Hz, 1H), 8.35 (s, 1H), 8.83 (s, 1H), 10.43 (s, 1H), 12.42 (br s, 1H). | ethyl (2E)-3- (3-{2-[(3- chloro-4- fluorophenyl) amino]-4-[3- (trifluoro- methyl)-1H- pyrazol-1- yl]pyrimidin- 5- yl}phenyl)- prop-2-enoate (Example 160) |
| 324 | 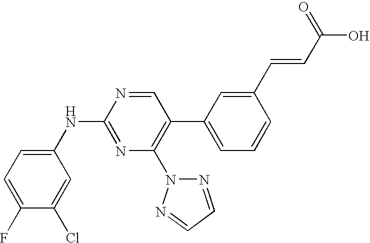 (2E)-3-(3-{2-[(3-chloro- 4-fluorophenyl)amino]-4- (2H-1,2,3-triazol-2- yl)pyrimidin-5- yl}phenyl)prop-2-enoic acid | MS(ES): 437 (M + 1) for C₂₁H₁₄ClFN₆O₂. ¹H NMR (400 MHz, DMSO- d₆): δ 6.50 (d, J = 16.40 Hz, 1H), 7.00 (d, J = 8.00 Hz, 1H), 7.34 (t, J = 7.60 Hz, 1H), 7.42 (t, J = 9.20 Hz, 1H), 7.46 (br s, 1H), 7.48 (d, J = 15.60 Hz, 1H), 7.61 (d, J = 8.00 Hz, 1H), 7.74-7.77 (m, 1H), 8.10 (s, 2H), 8.20 (dd, J = 2.40, 6.60 Hz, 1H), 8.88 (br s, 1H), 10.52 (br s, 1H). | ethyl (2E)-3- (3-{2-[(3- chloro-4- fluorophenyl) amino]-4- (2H-1,2,3- triazol-2- yl)pyrimidin- 5- yl}phenyl) prop-2-enoate (Example 191) |
| 325 | 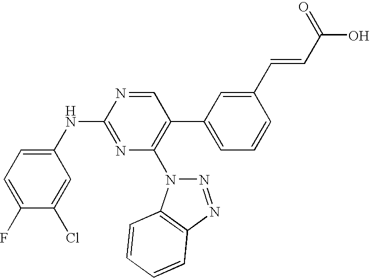 (2E)-3-(3-{4-(1H- benzotriazol-1-yl)-2-[(3- chloro-4- fluorophenyl)amino] pyrimidin-5-yl}phenyl) prop-2-enoic acid | MS(ES): 487 (M + 1) for C₂₅H₁₆ClFN₆O₂. ¹H NMR (400 MHz, DMSO- d₆): δ 6.40 (d, J = 16.00 Hz, 1H), 6.89 (d, J = 7.88 Hz, 1H), 7.23 (t, J = 7.80 Hz, 1H), 7.39-7.47 (m, 2H), 7.51-7.52 (m, 2H), 7.57 (d, J = 7.80 Hz, 1H), 7.72-7.75 (m, 1H), 7.93- 7.96 (m, 2H), 8.18 (dd, J = 2.68, 6.70 Hz, 1H), 9.03 (s, 1H), 10.64 (s, 1H). | ethyl (2E)-3- (3-{4-(1H- benzotriazol- 1-yl)-3-[(3- chloro-4- fluorophenyl) amino] pyrimidin-5- yl}phenyl) prop-2-enoate (Example 194) |
| 326 | 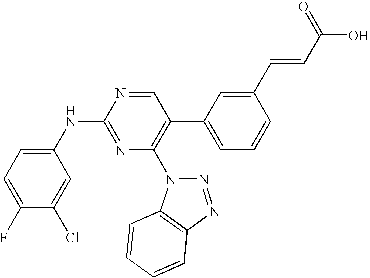 (2E)-3-(3-{4-(1H- benzotriazol-1-yl)-2-[(3- chloro-4- fluorophenyl)amino] pyrimidin-5-yl}phenyl) prop-2-enoic acid and 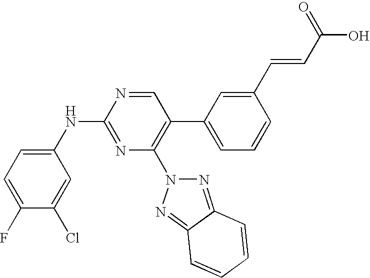 (2E)-3-(3-{4-(2H- benzotriazol-2-yl)-2-[(3- chloro-4- fluorophenyl)amino] pyrimidin-5-yl}phenyl) prop-2-enoic acid | MS(ES): 487 (M + 1) for C₂₅H₁₆ClFN₆O₂. Mixture of regioisomers: ¹H NMR (400 MHz, DMSO- d₆): δ 6.40 (d, J = 16.04 Hz, 1H), 6.46 (d, J = 16.00 Hz, 1H), 6.89 (d, J = 7.72 Hz, 1H), 7.10 (d, J = 7.96 Hz, 1H), 7.24 (t, J = 7.96 Hz, 1H), 7.30 (t, J = 7.64 Hz, 1H), 7.38-7.45 (m, 2H), 7.49-7.72 (m, 8H), 7.95 (dd, J = 3.04, 6.68 Hz, 1H), 8.13-8.19 (m, 3H), 8.93 (s, 1H), 9.03 (s, 1H), 12.5 (br s, 1H). | ethyl (2E)-3- (3-{4-(1H- benzotriazol- 1-yl)-2-[(3- chloro-4- fluorophenyl) amino] pyrimidin-5- yl}phenyl)- prop-2-enoate and ethyl (2E)-3- (3-{4-(2H- benzotriazol- 2-yl)-2-[(3- chloro-4- fluorophenyl) amino]- pyrimidin-5- yl}phenyl) prop-2-enoate |

### Example 327

### 5-{2-[(3-chloro-4-fluorophenyl)amino]-4-(4,5-dichloro-1H-imidazol-1-yl)pyrimidin-5-yl}pyridine-3-carboxylic acid

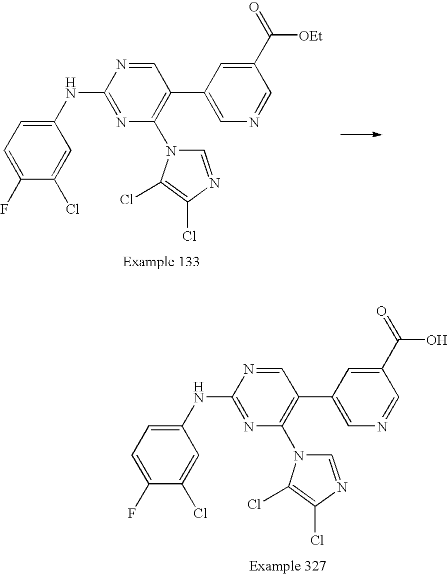

To a solution of ethyl 5-{2-[(3-chloro-4-fluorophenyl)amino]-4-(4,5-dichloro-1H-imidazol-1-yl)pyrimidin-5-yl}pyridine-3-carboxylate Example 133 (289 mg, 0.57 mmol) in 1,2-dichloroethane, was added trimethyltin hydroxide (10 eq, 5.7 mmol) and the mixture was heated to 80-85° C. until TLC analysis indicated a complete reaction. After completion of the reaction, the mixture was concentrated in vacuum and the residue was taken up in ethyl acetate (˜15 mL). The organic layer was washed with 5% HCl, brine and dried over anhydrous Na₂SO₄. Removal of the solvent afforded the crude carboxylic acid. The crude compound was purified by RP-HPLC (Kromosil C18 column) to provide (150 mg) of pure 5-{2-[(3-chloro-4-fluorophenyl)amino]-4-(4,5-dichloro-1H-imidazol-1-yl)pyrimidin-5-yl}pyridine-3-carboxylic acid.

MS(ES): 479 (M+1) for C₁₉H₁₀Cl₃FN₆O₂.

¹H NMR (400 MHz, DMSO-d₆): δ 7.42 (t, J=9.12 Hz, 1H), 7.69 (ddd, J=2.76, 4.04, 9.05 Hz, 1H), 7.97 (br s, 1H), 8.05 (ddd, J=1.92, 6.48 Hz, 1H), 8.12 (br s, 1H), 8.60 (br s, 1H), 9.01 (br s, 1H), 9.04 (s, 1H), 10.62 (s, 1H), 13.59 (br s, 1H).

The following examples were prepared using the general method described above for (Example 327) using trimethyltin hydroxide and the starting material (SM) indicated.

| Ex | Compound | Data | SM |
|---|---|---|---|
| 328 | 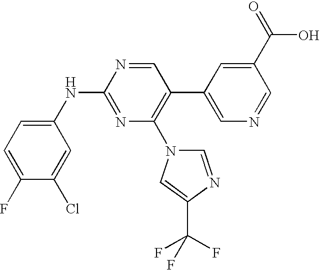 5-{2-[(3-chloro-4- fluorophenyl)amino]-4- [4-(trifluoromethyl)- 1H-imidazol-1- yl]pyrimidin-5- yl}pyridine-3- carboxylic acid | MS(ES): 479 (M + 1) for C₂₀H₁₁ClF₄N₆O₂. ¹H NMR (400 MHz, DMSO- d₆): δ 7.42 (t, J = 9.12 Hz, 1H), 7.70 (ddd, J = 2.76, 4.18, 9.08 Hz, 1H), 7.89 (t, J = 1.08 Hz, 1H), 8.01 (s, 1H), 8.06 (dd, J = 2.52, 6.76 Hz, 1H), 8.10 (t, J = 2.08 Hz, 1H), 8.65 (d, J = 2.16 Hz, 1H), 8.89 (s, 1H), 9.04 (d, J = 1.88 Hz, 1H), 10.49 (s, 1H). | ethyl 5-{2- [(3-chloro-4- fluorophenyl) amino]-4-[4- (trifluoro- methyl)-1H- imidazol-1- yl]pyrimidin- 5- yl}pyridine- 3-carboxylate (Example 204) |
| 329 | 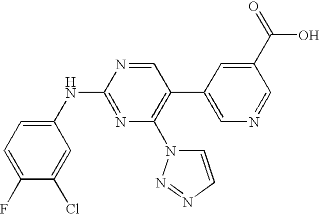 5-{2-[(3-chloro-4- fluorophenyl)amino]-4- (1H-1,2,3-triazol-1- yl)pyrimidin-5- yl}pyridine-3- carboxylic acid | MS(ES): 412 (M + 1) for C₁₈H₁₁ClFN₇O₂. ¹H NMR (400 MHz, DMSO- d₆): δ 7.42 (t, J = 8.80 Hz, 1H), 7.73-7.75 (m, 1H), 7.94 (s, 1H), 8.03 (m, 2H), 8.56 (s, 1H), 8.61 (s, 1H), 8.89 (s, 1H), 8.99 (s, 1H), 10.51 (br s, 1H). | ethyl 5-{2- [(3-chloro-4- fluorophenyl) amino]-4- (1H-1,2,3- triazol-1- yl)pyrimidin- 5- yl}pyridine- 3-carboxylate (Example 207) |
| 330 | 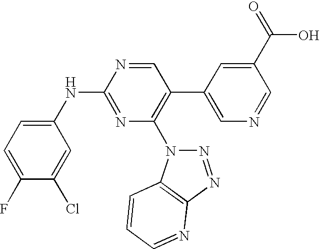 5-{2-[(3-chloro-4- fluorophenyl)amino]-4- (1H-[1,2,3]triazolo[4,5- b]pyridin-1- yl)pyrimidin-5- yl}pyridine-3- carboxylic acid and 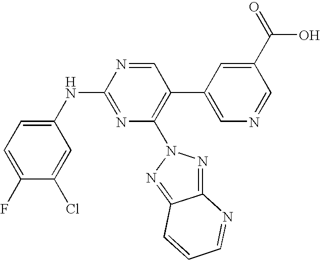 5-{2-[(3-chloro-4- fluorophenyl)amino]-4- (2H-[1,2,3]triazolo[4,5- b]pyridin-2- yl)pyrimidin-5- yl}pyridine-3- carboxylic acid | MS(ES): 461 (M − 1) for C₂₁H₁₂ClFN₈O₂. Mixture of regioisomers: ¹H NMR (400 MHz, DMSO- d₆): δ 7.42 (t, J = 9.08 Hz, 1H), 7.56-7.59 (m, 2H), 7.78-7.81 (m, 2H), 7.95 (s, 1H), 8.11-8.13 (m, 2H), 8.18-8.20 (m, 1H), 8.43 (br s, 1H), 8.48-8.50 (m, 1H), 8.64 (s, 1H), 8.65-8.82 (m, 2H), 8.90-8.94 (m, 3H), 8.99- 9.02 (m, 2H), 10.48 (br s, 1H), 10.71 (br s, 1H). | ethyl 5-{2- [(3-chloro-4- fluorophenyl) amino]-4- (1H- [1,2,3]triazolo- [4,5- b]pyridin-1- yl)pyrimidin- 5- yl}pyridine- 3-carboxylate and ethyl 5-{2- [(3-chloro-4- fluorophenyl) amino]-4- (2H- [1,2,3]triazolo- [4,5- b]pyridin-2- yl)pyrimidin- 5- yl}pyridine- 3-carboxylate (Example 208) |
| 331 | 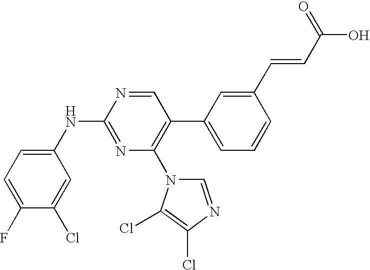 (2E)-3-(3-{2-[(3- chloro-4- fluorophenyl)amino]-4- (4,5-dichloro-1H- imidazol-1- yl)pyrimidin-5- yl}phenyl)prop-2-enoic acid | MS(ES): 504 (M + 1) and 506 (M + 3) for C₂₂H₁₃Cl₃FN₅O₂. ¹H NMR (400 MHz, DMSO- d₆): δ 6.51 (d, J = 16.04 Hz, 1H), 7.10 (d, J = 7.80 Hz, 1H), 7.39 (d, J = 1.76 Hz, 1H), 7.41 (t, J = 8.76 Hz, 1H), 7.54 (d, J = 16.00 Hz, 1H), 7.55 (s, 1H), 7.66-7.70 (m, 2H), 8.04-8.07 (m, 2H), 8.97 (s, 1H), 10.54 (s, 1H). | ethyl (2E)-3- (3-{2-[(3- chloro-4- fluorophenyl) amino]-4- (4,5-dichloro- 1H-imidazol- 1- yl)pyrimidin- 5- yl}phenyl) prop-2-enoate (Example 161) |
| 332 | 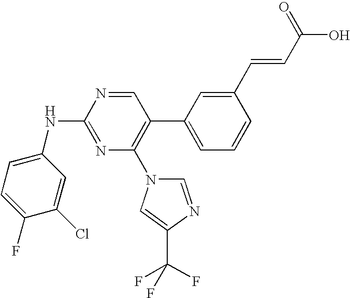 (2E)-3-(3-{2-[(3- chloro-4- fluorophenyl)amino]-4- [4-(trifluoromethyl)- 1H-imidazol-1- yl]pyrimidin-5- yl}phenyl)prop-2-enoic acid | MS(ES): 504 (M + 1) for C₂₃H₁₄ClF₄N₅O_{2.} ¹H NMR (400 MHz, DMSO- d₆): δ 6.52 (d, J = 16.00 Hz, 1H), 7.24 (d, J = 7.84 Hz, 1H), 7.39-7.47 (m, 2H), 7.56 (d, J = 16.00 Hz, 1H), 7.64 (s, 1H), 7.67-7.72 (m, 2H), 7.80 (d, J = 1.08 Hz, 1H), 7.94 (s, 1H), 8.07 (dd, J = 2.52, 6.62 Hz, 1H), 8.82 (s, 1H), 10.42 (s, 1H), 12.47 (br s, 1H). | ethyl (2E)-3- (3-{2-[(3- chloro-4- fluorophenyl) amino]-4-[4- (trifluoro- methyl)-1H- imidazol-1- yl]pyrimidin- 5- yl}phenyl)- prop-2-enoate (Example 189) |
| 333 | 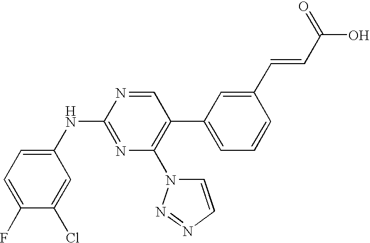 (2E)-3-(3-{2-[(3- chloro-4- fluorophenyl)amino]-4- (1H-1,2,3-triazol-1- yl)pyrimidin-5- yl}phenyl)prop-2-enoic acid | MS(ES): 437 (M + 1) for C₂₁H₁₄ClFN₆O₂. ¹H NMR (400 MHz, DMSO- d₆): δ 6.49 (d, J = 16.04 Hz, 1H), 7.09 (d, J = 7.76 Hz, 1H), 7.36 (t, J = 7.76 Hz, 1H), 7.41 (t, J = 9.08 Hz, 1H), 7.52 (d, J = 15.56 Hz, 1H), 7.54 (s, 1H), 7.63 (d, J = 7.84 Hz, 1H), 7.70- 7.74 (m, 1H), 7.92 (d, J = 0.88 Hz, 1H), 8.05-8.09 (m, 1H), 8.51 (s, 1H), 8.89 (s, 1H), 10.48 (s, 1H), 12.45 (br s, 1H). | ethyl (2E)-3- (3-{2-[(3- chloro-4- fluorophenyl) amino]-4- (1H-1,2,3- triazol-1- yl)pyrimidin- 5- yl}phenyl) prop-2-enoate (Example 192) |
| 334 | 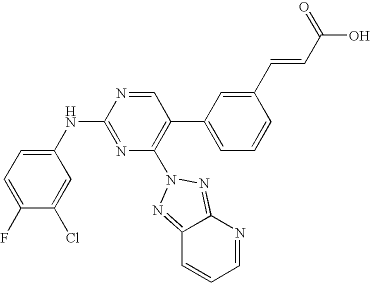 (2E)-3-(3-{2-[(3- chloro-4- fluorophenyl)amino]-4- (2H-[1,2,3]triazolo[4,5- b]pyridin-2- yl)pyrimidin-5- yl}phenyl)prop-2-enoic acid | MS(ES): 486 (M − 1) for C₂₄H₁₅ClFN₇O₂. ¹H NMR (400 MHz, DMSO- d₆): δ 6.42 (d, J = 16.40 Hz, 1H), 6.94 (d, J = 8.00 Hz, 1H), 7.26 (t, J = 8.00 Hz, 1H), 7.43 (t, J = 9.20 Hz, 1H), 7.49 (d, J = 16.00 Hz, 1H), 7.58-7.61 (m, 3H), 7.75-7.77 (m, 1H), 8.18 (dd, J = 2.40, 6.40 Hz, 1H), 8.52 (d, J = 8.40 Hz, 1H), 8.92 (d, J = 2.80 Hz, 1H), 9.05 (br s, 1H), 10.68 (s, 1H), 12.42 (br s, 1H). | ethyl (2E)-3- (3-{2-[(3- chloro-4- fluorophenyl) amino]-4- (2H- [1,2,3]triazolo [4,5- b]pyridin-2- yl)pyrimidin- 5- yl}phenyl) prop-2-enoate (Example 193) |

The following examples were prepared using the general method described for Example 1 using the starting materials (SM) indicated.

| Ex | Compound | Data | SM |
|---|---|---|---|
| 335 | 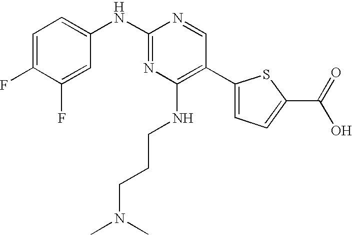 5-(2-(3,4- difluorophenyl amino)-4-(3- (dimethyl amino)propylamino) pyrimidin-5- yl)thiophene-2- carboxylic acid | MS: ES+ 434 for C₂₀H₂₁F₂N₅O₂S ¹H NMR (300 MHz, DMSO-d₆) δ ppm 1.86-2.02 (m, 2 H) 2.75 (d, J = 4.52 Hz, 6 H) 2.98-3.11 (m, 2 H) 3.44 (q, J = 6.15 Hz, 2 H) 7.27 (d, J = 3.96 Hz, 1 H) 7.35-7.46 (m, 2 H) 7.57 (br. s., 1 H) 7.76 (d, J = 3.96 Hz, 1 H) 7.83-7.97 (m, 1 H) 8.04 (s, 1 H) 9.58 (br. s., 1 H) 10.19 (br. s., 1 H) | 5-borono thiophene-2- carboxylic acid and 5-Bromo-N²- (3,4-difluoro- phenyl)-N⁴- (3- dimethyl- amino-propyl)- pyrimidine- 2,4-diamine hydrochloride salt (Intermediate 27) |
| 336 | 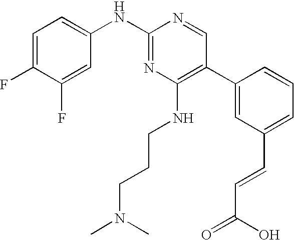 (E)-3-(3-(2-(3,4- difluoro phenylamino)-4-(3- (dimethylamino) propylamino) pyrimidin- 5-yl)phenyl)acrylic acid | MS: ES 454 for C₂₄H₂₅F₂N₅O₂ ¹H NMR (300 MHz, DMSO-d₆) δ ppm 1.85-2.00 (m, 2 H) 2.75 (d, J = 4.71 Hz, 6 H) 3.04 (ddd, J = 10.13, 5.27, 5.13 Hz, 2 H) 3.42 (q, J = 6.09 Hz, 2 H) 6.61 (d, 1 H) 7.28-8.01 (m, 10 H) 9.57 (br. s., 1 H) 10.50 (br. s., 1 H) | (E)-3-(3- boronophenyl) acrylic acid and 5-Bromo-N²- (3,4-difluoro- phenyl)-N⁴- (3- dimethyl- amino-propyl)- pyrimidine- 2,4-diamine hydrochloride salt (Intermediate 27) |
| 337 | 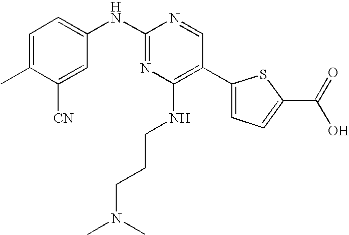 5-(2-(3-cyano-4- methyl phenylamino)-4-(3- (dimethylamino) propyl amino)pyrimidin-5- yl) thiophene-2- carboxylic acid | MS: ES+ 437 for C₂₂H₂₄N₆O₂S ¹H NMR (300 MHz, DMSO-d₆) δ ppm 1.87-2.05 (m, 2 H) 2.43 (s, 3 H) 2.76 (d, J = 4.71 Hz, 6 H) 3.08 (dt, J = 10.31, 5.11 Hz, 2 H) 3.45 (q, J = 6.22 Hz, 2 H) 7.26 (d, J = 3.96 Hz, 1 H) 7.34-7.50 (m, 2 H) 7.69-7.81 (m, 2 H) 8.04 (s, 1 H) 8.28 (d, J = 2.26 Hz, 1 H) 9.47 (br. s., 1 H) 10.03 (br. s., 1 H) | 5-borono thiophene-2- carboxylic acid and 5-[5-Bromo- 4-(3- dimethylamino- propylamino)- pyrimidin-2- ylamino]-2- methyl- benzonitrile (Intermediate 28) |
| 338 | 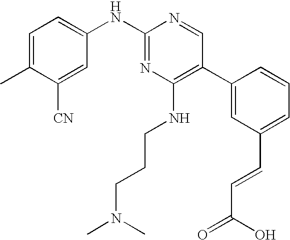 (E)-3-(3-(2-(3-cyano- 4- methylphenylamino)- 4-(3-(dimethylamino) propylamino) pyrimidin-5-yl) phenyl)acrylic acid | MS: ES+ 457 for C₂₆H₂₈N₆O₂ ¹H NMR (300 MHz, DMSO-d₆) δ ppm 1.88-2.02 (m, 2 H) 2.45 (s, 3 H) 2.76 (d, J = 4.71 Hz, 6 H) 3.07 (dt, J = 10.27, 5.23 Hz, 2 H) 3.43 (q, J = 6.03 Hz, 2 H) 6.61 (d, 1 H) 7.35-7.83 (m, 8 H) 7.91 (s, 1 H) 8.25 (d, J = 2.07 Hz, 1 H) 9.53 (br. s., 1 H) 10.31 (br. s., 1 H) | (E)-3-(3- boronophenyl) acrylic acid and 5-[5-Bromo- 4-(3- dimethylamino- propylamino)- pyrimidin-2- ylamino]-2- methyl- benzonitrile (Intermediate 28) |
| 339 | 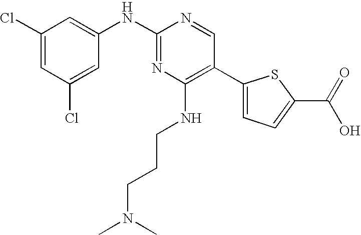 5-(2-(3,5-dichloro- phenylamino)-4-(3- (dimethylamino) propyl amino)pyrimidin-5- yl) thiophene-2- carboxylic acid | MS: ES+ 466 for C₂₀H₂₁Cl₂N₅O₂S ¹H NMR (300 MHz, DMSO-d₆) δ ppm 1.90-2.05 (m, 2 H) 2.76 (d, J = 4.71 Hz, 6 H) 3.09 (ddd, J = 10.31, 5.37, 5.23 Hz, 2 H) 3.40- 3.52 (m, 2 H) 7.12 (t, 1 H) 7.20- 7.31 (m, 2 H) 7.76 (d, J = 3.77 Hz, 1 H) 7.90 (d, J = 1.88 Hz, 2 H) 8.04 (s, 1 H) 9.37 (br. s., 1 H) 9.89 (s, 1 H) | 5-borono- thiophene-2- carboxylic acid and 5-Bromo-N²- (3,5-dichloro- phenyl)-N⁴- (3- dimethylamino- propyl)- pyrimidine- 2,4-diamine- hydrochloride salt (Intermediate 32) |
| 340 | 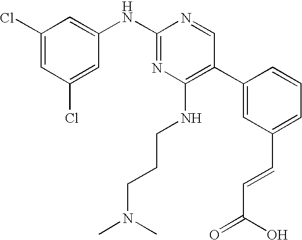 (E)-3-(3-(2-(3,5- dichloro phenylamino)-4-(3- (dimethylamino) propyl amino)pyrimidin-5- yl) phenyl)acrylic acid | MS: ES+ 486 for C₂₄H₂₅Cl₂N₅O₂ ¹H NMR (300 MHz, DMSO-d₆) δ ppm 1.90-2.04 (m, 2 H) 2.76 (d, J = 4.71 Hz, 6 H) 3.08 (dt, J = 10.22, 5.16 Hz, 2 H) 3.44 (q, J = 5.53 Hz, 2 H) 6.61 (d, 1 H) 7.18 (s, 1 H) 7.32-7.43 (m, 1 H) 7.43-7.49 (m, 1 H) 7.53 (t, J = 7.82 Hz, 1 H) 7.64 (d, J = 16.01 Hz, 1 H) 7.71- 7.77 (m, 2 H) 7.87 (d, J = 1.70 Hz, 2 H) 7.92 (s, 1 H) 9.45 (br. s., 1 H) 10.16 (br. s., 1 H) | (E)-3-(3- boronophenyl) acrylic acid and 5-Bromo-N²- (3,5-dichloro- phenyl)-N⁴- (3- dimethylamino- propyl)- pyrimidin- 2,4-diamine hydrochloride salt (Intermediate 32) |
| 341 | 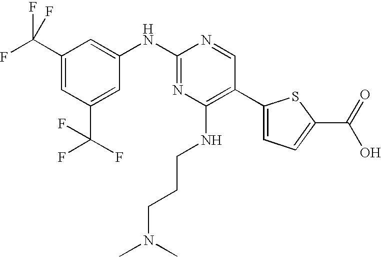 5-(2-(3,5-bis(trifluoro methyl)phenylamino)- 4-(3- (dimethylamino) propyl amino)pyrimidin-5- yl) thiophene-2- carboxylic acid | MS: ES+ 534 for C₂₂H₂₁F₆N₅O₂S ¹H NMR (300 MHz, DMSO-d₆) δ ppm 1.88-2.02 (m, 2 H) 2.74 (d, J = 4.52 Hz, 6 H) 2.99-3.12 (m, 2 H) 3.42-3.56 (m, 2 H) 7.19- 7.33 (m, 2 H), 7.59 (s, 1 H) 7.76 (d, J = 3.77 Hz, 1 H) 8.07 (s, 1 H) 8.51 (s, 2 H) 9.47 (br. s., 1 H) 10.20 (s, 1 H) | 5-borono thiophene-2- carboxylic acid and N²-(3,5-Bis- trifluoro- methyl-phenyl)-5- bromo-N⁴-(3- dimethylamino- propyl)- pyrimidine- 2,4-diamine (Intermediate 33) |
| 342 | 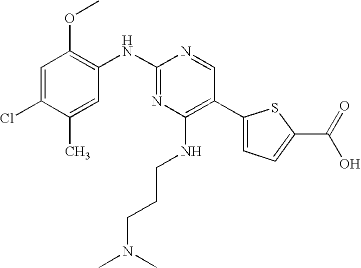 5-(2-(4-chloro-2- methoxy-5- methylphenylamino)- 4-(3- (dimethylamino) propyl amino)pyrimidin-5- yl) thiophene-2- carboxylic acid | MS: ES+ 476 for C₂₂H₂₆ClN₅O₃S ¹H NMR (300 MHz, DMSO-d₆) δ ppm 1.83-1.98 (m, 2 H) 2.28 (s, 3 H) 2.74 (d, J = 4.52 Hz, 6 H) 2.94-3.08 (m, 2 H) 3.42 (q, J = 5.40 Hz, 2 H) 3.85 (s, 3 H) 7.17 (s, 1 H) 7.29 (d, J = 3.77 Hz, 1 H) 7.77 (d, J = 3.77 Hz, 1 H) 7.80- 7.88 (m, 1 H) 7.93 (br. s., 1 H) 8.00 (s, 1 H) 9.04 (br. s., 1 H) 9.56 (br. s., 1 H) | 5-borono thiophene-2- carboxylic acid and 5-Bromo-N²- (4-chloro-2- methoxy-5- methyl- phenyl)-N⁴- (3- dimethylamino- propyl)- pyrimidine- 2,4-diamine (Intermediate 29) |
| 343 | 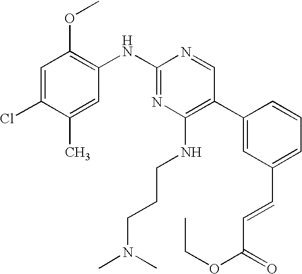 (E)-ethyl 3-(3-(2-(4- chloro-2-methoxy-5- methyl phenylamino)-4-(3- (dimethylamino) propyl amino)pyrimidin-5- yl) phenyl)acrylate | MS: ES+ 524 for C₂₈H₃₄ClN₅O₃ ¹H NMR (300 MHz, DMSO-d₆) δ ppm 1.25 (t, J = 7.06 Hz, 3 H) 1.82- 1.97 (m, 2 H) 2.30 (s, 3 H) 2.73 (d, J = 4.52 Hz, 6 H) 3.00 (dt, J = 9.70, 4.95 Hz, 2 H) 3.32-3.46 (m, 2 H) 3.86 (s, 3 H) 4.19 (q, J = 7.10 Hz, 2 H) 6.73 (d, J = 16.01 Hz, 1 H) 7.24 (s, 1 H) 7.43-7.62 (m, 2 H) 7.65-7.90 (m, 5 H) 8.18 (t, J = 4.71 Hz, 1 H) 9.78 (br. s., 2 H) | (E)-3-(3- ethoxy-3-oxo prop-1-enyl) phenyl- boronic acid and 5-Bromo-N²- (4-chloro-2- methoxy-5- methyl- phenyl)-N⁴- (3- dimethylamino- propyl)- pyrimidine- 2,4-diamine (Intermediate 29) |
| 344 | 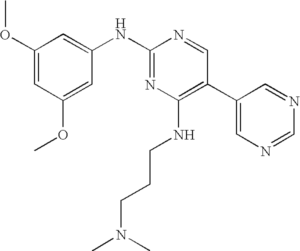 N²-(3,5-dimethoxy phenyl)-N⁴-(3- (dimethyl amino)propyl)-5,5′-bi pyrimidin-2,4- diamine | MS: ES+ 410 for C₂₁H₂₇N₇O₂ ¹H NMR (300 MHz, DMSO-d₆) δ ppm 1.84-2.00 (m, 2 H) 2.74 (d, J = 4.71 Hz, 6 H) 2.95-3.13 (m, 2 H) 3.43 (q, 2 H) 3.75 (s, 6 H) 6.31 (s, 1 H) 6.90 (d, J = 1.70 Hz, 2 H) 7.98 (s, 1 H) 8.10 (br. s., 1 H) 8.86 (s, 2 H) 9.25 (s, 1 H) 9.65 (br. s., 1 H) 10.36 (br. s., 1 H) | pyrimidin-5- yl boronic acid and 5-Bromo-N²- (3,5- dimethoxy- phenyl)-N⁴- (3- dimethylamino- propyl)- pyrimidine- 2,4-diamine hydrochloride salt (Intermediate 34) |
| 345 | 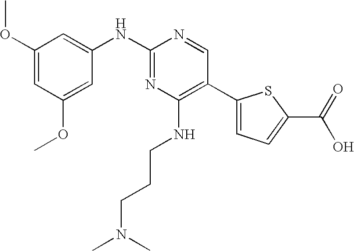 5-(2-(3,5-dimethoxy- phenylamino)-4-(3- (dimethylamino) propy amino)pyrimidin-5- yl) thiophene-2- carboxylic acid | MS: ES+ 458 for C₂₂H₂₇N₅O₄S ¹H NMR (300 MHz, DMSO-d₆) δ ppm 1.88-2.02 (m, 2 H) 2.74 (d, 6 H) 3.06 (dt, J = 10.31, 5.11 Hz, 2 H) 3.46 (q, J = 6.15 Hz, 2 H) 3.73 (s, 6 H) 6.21 (t, J = 1.98 Hz, 1 H) 6.97 (d, J = 2.07 Hz, 2 H) 7.26 (d, J = 3.96 Hz, 1 H) 7.46 (br. s., 1 H) 7.76 (d, J = 3.77 Hz, 1 H) 8.00 (s, 1 H) 9.46 (br. s., 1 H) 9.78 (br. s., 1 H) | 5-borono- thiophene-2- carboxylic acid and 5-Bromo-N²- (3,5- dimethoxy- phenyl)-N⁴- (3- dimethylamino- propyl)- pyrimidine- 2,4-diamine hydrochloride salt (Intermediate 34) |
| 346 | 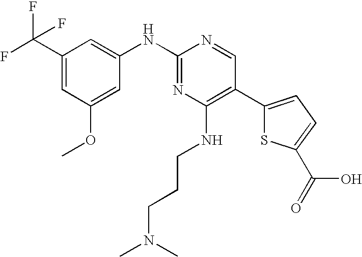 5-{4-[3- (dimethylamino) propylamino]-2-[3- methoxy-5- (trifluoromethyl) phenylamino] pyrimidin-5-yl}thiophene- 2-carboxylic acid | MS: ES+ 496 for C₂₂H₂₄F₃N₅O₃S ¹H NMR (300 MHz, DMSO-d₆) δ ppm 1.88-2.02 (m, 2 H) 2.74 (d, J = 4.71 Hz, 6 H) 3.06 (dt, J = 10.41, 5.25 Hz, 2 H) 3.47 (q, J = 5.53 Hz, 2 H) 3.82 (s, 3 H) 6.84 (s, 1 H) 7.23-7.24 (m, 2 H) 7.62 (s, 1H) 7.76 (d, J = 3.77 Hz, 1 H) 7.86 (s, 1 H) 8.04 (s, 1 H) 9.39 (br. s., 1 H) 9.92 (s, 1 H) | 5-borono thiophene-2- carboxylic acid and 5-Bromo-N⁴- (3- dimethylamino- propyl)-N²- (3-methoxy- 5- trifluoromethyl- phenyl)- pyrimidine- 2,4-diamine (Intermediate 31) |
| 347 | 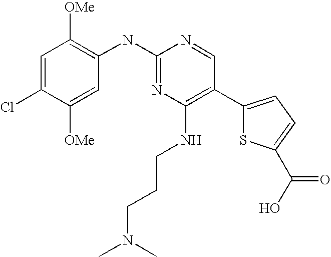 5-(2-(4-chloro-2,5- dimethoxyphenyl- amino)-4-(3- (dimethylamino) propylamino)pyrimidin- 5-yl)thiophene-2- carboxylic acid | MS: ES+ 492 for C₂₂H₂₆ClN₅O₄S ¹H NMR (300 MHz, DMSO-d₆) δ ppm 1.84-1.97 (m, 2 H) 2.73 (d, J = 4.52 Hz, 6 H) 2.94-3.06 (m, 2 H) 3.46 (q, J = 6.09 Hz, 2 H) 3.82 (d, J = 6.59 Hz, 6 H) 7.20 (s, 1 H) 7.28 (d, J = 3.77 Hz, 1 H) 7.64 (br. s., 1 H) 7.77 (d, J = 3.96 Hz, 1 H) 7.95 (br. s., 1 H) 8.02 (s, 1 H) 8.76 (br. s., 1 H) 9.45 (br. s., 1 H) 13.30 (br. s., 1 H) | 5-borono thiophene-2- carboxylic acid and 5-Bromo-N²- (4-chloro- 2,5- dimethoxy- phenyl)-N⁴- (3- dimethylamino- propyl)- pyrimidine- 2,4-diammine (Intermediate 30) |
| 348 | 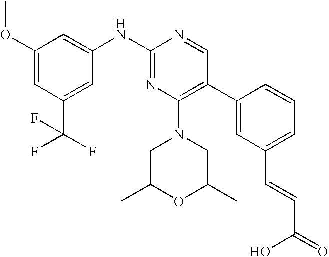 (E)-3-(3-(4-(2,6- dimethylmorpholino)- 2-(3-methoxy-5- (trifluoro methyl)phenylamino) pyrimidin-5- yl)phenyl) acrylic acid | MS: ES+ 529 for C₂₇H₂₇F₃N₄O₄ ¹H NMR (300 MHz, DMSO-d₆) δ ppm 0.91 (d, J = 6.22 Hz, 6 H) 2.50- 2.59 (m, 2 H) 3.44-3.71 (m, 4 H) 3.82 (s, 3 H) 6.62 (d, J = 16.01 Hz, 1 H) 6.84 (s, 1 H) 7.50 (t, J = 4.62 Hz, 3 H) 7.63-7.71 (m, 2 H) 7.78 (s, 1 H) 7.92 (s, 1H) 8.08 (s, 1 H) 9.95 (s, 1 H) | (E)-3-(3- boronophenyl) acrylic acid and 5-bromo-4- (2,6- dimethyl- morpholino)-N- (3-methoxy- 5-(trifluoro methyl)phenyl) pyrimidin-2- amine (Intermediate 120) |
| 349 | 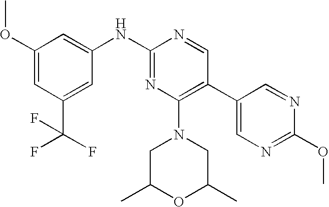 4-(2,6-dimethyl morpholino)-2′- methoxy-N-(3- methoxy-5- (trifluoromethyl) phenyl)-5,5′-bipyrimidin- 2-amine | MSP: ES+ 491 for C₂₃H₂₅F₃N₆O₃ ¹H NMR (300 MHz, DMSO-d₆) δ ppm 0.98 (d, J = 6.22 Hz, 6 H) 2.51- 2.57 (m, 2 H) 3.49-3.67 (m, 4 H) 3.80 (s, 3 H) 3.95 (s, 3 H) 6.79 (s, 1 H) 7.57 (s, 1 H) 7.95 (s, 1 H) 8.11 (s, 1 H) 8.71 (s, 2 H) 9.80 (s, 1 H) | 2-methoxy pyrimidin- 5-ylboronic acid and 5-bromo-4- (2,6- dimethyl- morpholino)-N- (3-methoxy- 5-(trifluoro methyl)phenyl) pyrimidin-2- amine (Intermediate 120) |
| 350 | 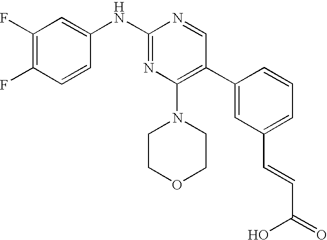 (E)-3-(3-(2-(3,4- difluoro phenylamino)-4- morpholinopyrimidin- 5-yl) phenyl)acrylic acid | MS: ES+ 439 for C₂₃H₂₀F₂N₄O₃ ¹H NMR (300 MHz, DMSO-d₆) δ ppm 3.27 (br. s., 4 H) 3.55 (br. s., 4 H) 6.61 (d, J = 16.01 Hz, 1 H) 7.27-7.72 (m, 7 H) 7.78 (s, 1 H) 7.83-7.97 (m, 1 H) 8.06 (s, 1 H) 9.86 (br. s., 1 H) | (E)-3-(3- boronophenyl) acrylic acid and 5-bromo-N- (3,4- difluoro- phenyl)-4- morpholino- pyrimidin-2- amine (Intermediate 122) |
| 351 | 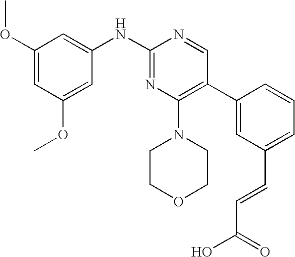 (E)-3-(3-(2-(3,5- dimethoxy phenylamino)-4- morpholino pyrimidin-5- yl)phenyl) acrylic acid | MS: ES+ 463 for C₂₅H₂₆N₄O₅ ¹H NMR (300 MHz, DMSO-d₆) δ ppm 3.33-3.42 (m, 4 H) 3.56 (d, 4 H) 3.74 (s, 6 H) 6.23 (t, J = 2.07 Hz, 1 H) 6.63 (d, J = 16.01 Hz, 1 H) 6.86-6.96 (m, 2 H) 7.51 (d, J = 4.71 Hz, 2 H) 7.58-7.73 (m, 2 H) 7.79 (s, 1 H) 8.03 (s, 1 H) 9.99 (br. s., 1 H) | (E)-3-(3- boronophenyl) acrylic acid and 5-chloro-N- (3,5- dimethoxy- phenyl)-4- morpholino- pyrimidin-2- amine (Intermediate 59) |
| 352 | 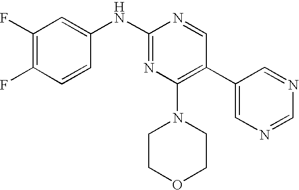 N-(3,4- difluorophenyl)-4- morpholino-5,5′- bipyrimidin-5-amine | MS: ES+ 371 for C₁₈H₁₆F₂N₆O ¹H NMR (300 MHz, DMSO-d₆) δ ppm 3.19-3.28 (m, 4 H) 3.52- 3.62 (m, 4 H) 7.28-7.41 (m, 1 H) 7.42-7.51 (m, 1 H) 7.96 (ddd, J = 14.08, 7.49, 2.35 Hz, 1 H) 8.17 (s, 1 H) 8.94 (s, 2 H) 9.13 (s, 1 H) 9.76 (s, 1 H) | pyrimidin-5- yl boronic acid and 5-bromo-N- (3,4- difluorophenyl)- 4- morpholino- pyrimidin-2- amine (Intermediate 122) |
| 353 | 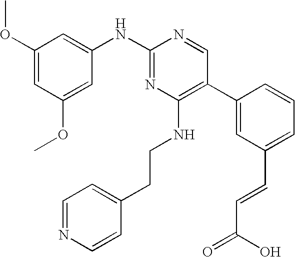 (E)-3-(3-(2-(3,5- dimethoxyphenyl- amino)-4-(2-(pyridin-4- yl) ethylamino) pyrimidin-5- yl)phenyl) acrylic acid | MS: ES+ 498 for C₂₈H₂₇N₅O₄ ¹H NMR (300 MHz, DMSO-d₆) δ ppm 3.08 (t, 2 H) 3.64-3.78 (m, 8 H) 6.35 (t, 1 H) 6.59 (d, J = 16.01 Hz, 1 H) 6.83 (d, J = 2.07 Hz, 2 H) 7.37 (d, J = 7.72 Hz, 1 H) 7.53 (t, J = 7.72 Hz, 1 H) 7.57-7.71 (m, 4 H) 7.78 (d, J = 7.91 Hz, 1 H) 7.87 (s, 1 H) 8.07 (br. s., 1 H) 8.70 (d, J = 6.40 Hz, 2 H) 10.46 (s, 1 H) | (E)-3-(3- boronophenyl) acrylic acid and 5-bromo-N²- (3,5- dimethoxy- phenyl)-N⁴-(2- (pyridin-4- yl)ethyl) pyrimidine- 2,4-diamine (Intermediate 57) |
| 354 | 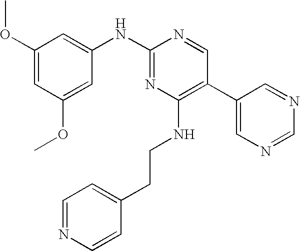 N²-(3,5- dimethoxyphenyl)- N⁴-(2-(pyridin-4- yl)ethyl)-5,5′- bipyrimidine-2,4- diamine | MS: ES+ 430 for C₂₃H₂₃N₇O₂ ¹H NMR (300 MHz, DMSO-d₆) δ ppm 3.10 (t, 2 H) 3.63-3.78 (m, 8 H) 6.32 (t, J = 1.98 Hz, 1 H) 6.86 (d, J = 2.07 Hz, 2 H) 7.74 (d, J = 6.22 Hz, 2 H) 7.97 (s, 1 H) 8.12 (br. s., 1 H) 8.65-8.83 (m, 4 H) 9.24 (s, 1 H) 10.36 (br. s., 1 H) | pyrimidin-5- ylboronic acid and 5-bromo-N²- (3,5- dimethoxy- phenyl)-N⁴-(2- (pyridin-4- yl)ethyl) pyrimidine- 2,4-diamine (Intermediate 57) |
| 355 | 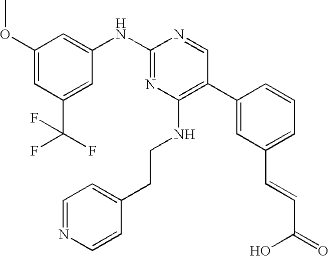 (E)-3-(3-(2-(3- methoxy-5- (trifluoromethyl) phenyl amino)-4-(2-(pyridin- 4-yl) ethylamino) pyrimidin-5- yl)phenyl) acrylic acid | MS: ES+ 536 for C₂₈H₂₄F₃N₅O₃ ¹H NMR (300 MHz, DMSO-d₆) δ ppm 3.11 (t, 2 H) 3.72 (q, J = 6.41 Hz, 2 H) 3.81 (s, 3 H) 6.57 (d, J = 16.01 Hz, 1 H) 6.95 (s, 1 H) 7.36 (d, J = 7.91 Hz, 1 H) 7.44- 7.80 (m, 10 H) 7.92 (s, 1 H) 8.72 (d, J = 6.41 Hz, 2 H) 10.46 (br. s., 1 H) | (E)-3-(3- boronophenyl) acrylic acid and 5-bromo-N²- (3-methoxy- 5- (trifluoro- methyl)phenyl)- N⁴-(2- (pyridin-4-yl) ethyl)pyrimidine- 2,4- diamine (Intermediate 121) |
| 356 | 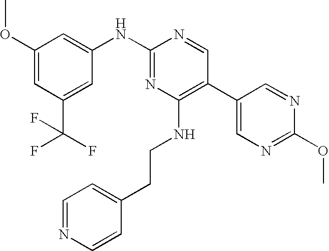 2′-methoxy-N²-(3- methoxy-5- (trifluoromethyl) phenyl)-N⁴-(2-(pyridin-4- yl)ethyl)-5,5′- bipyrimidine-2,4- diamine | MS: ES+ 498 for C₂₄H₂₂F₃N₇O₂ ¹H NMR (300 MHz, DMSO-d₆) δ ppm 3.14 (t, 2 H) 3.71 (q, J = 5.97 Hz, 2 H) 3.79-3.87 (m, 3 H) 3.95 (s, 3 H) 7.01 (s, 1 H) 7.47 (s, 1 H) 7.73 (s, 1 H) 7.85 (d, J = 6.41 Hz, 2 H) 8.01 (s, 1 H) 8.19 (br. s., 1 H) 8.49-8.58 (m, 2 H) 8.81 (d, J = 6.40 Hz, 2 H) 11.08 (br. s., 1 H) | 2-methoxy pyrimidin-5- ylboronic acid and 5-bromo-N²- (3-methoxy- 5- (trifluoro- methyl)phenyl)- N⁴-(2- (pyridin-4-yl) ethyl) pyrimidine- 2,4- diamine (Intermediate 121) |
| 357 | 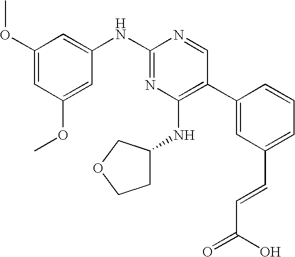 (R,E)-3-(3-(2-(3,5- dimethoxyphenylamino)- 4- (tetrahydrofuran-3- ylamino)pyrimidin-5- yl)phenyl)acrylic acid | MS: ES+ 463 for C₂₅H₂₆N₄O₅. ¹H NMR (300 MHz, DMSO-d₆) δ ppm 1.90-2.05 (m, 1 H) 2.08- 2.23 (m, 1 H) 3.49-3.93 (m, 10 H) 4.57-4.77 (m, 1 H) 6.32 (t, 1 H) 6.60 (d, J = 16.01 Hz, 1 H) 6.77- 6.91 (m, 2 H) 7.35-8.03 (m, 7 H) 10.24 (br. s., 1 H) | (E)-3-(3- boronophenyl) acrylic acid and (R)-5-bromo- N²-(3,5- dimethoxy- phenyl)-N⁴- (tetrahydro- furan-3-yl) pyrimidine- 2,4-diamine (Intermediate 58) |
| 358 | 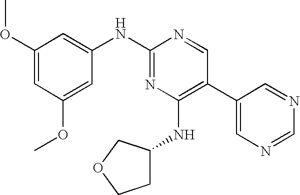 (R)-N²-(3,5- dimethoxyphenyl)- N⁴-(tetrahydrofuran- 3-yl)-5,5′- bipyrimidine-2,4- diamine | MS: ES+ 395 for C₂₀H₂₂N₆O₃. ¹H NMR (300 MHz, DMSO-d₆) δ ppm 1.86-2.00 (m, 1 H) 2.07- 2.30 (m, J = 12.72, 6.88 Hz, 1 H) 3.52-3.95 (m, 9 H) 4.55-4.71 (m, 2 H) 6.29 (s, 1 H) 6.89 (s, 2 H) 7.92 (s, 1 H) 7.97-8.12 (m, 1 H) 8.81 (s, 2 H) 9.23 (s, 1 H) 10.24 (s, 1 H) | pyrimidin-5- ylboronic acid and (R)-5-bromo- N²-(3,5- dimethoxy- phenyl)-N⁴- (tetrahydro- furan-3-yl) pyrimidine- 2,4-diamine (Intermediate 58) |
| 359 | 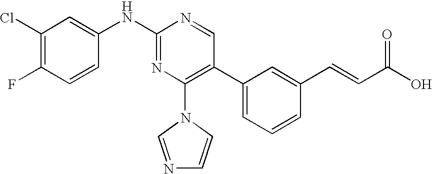 (E)-3-(3-(2-(3-chloro- 4- fluorophenylamino)- 4-(1H-imidazol-1- yl)pyrimidin-5- yl)phenyl)acrylic acid | MS(ES): 436 (M + 1) for C₂₂H₁₅ClFN₅O₂ ¹H NMR (300 MHz, DMSO-d6) δ ppm 6.53 (d, J = 16.01 Hz, 1 H) 6.98 (s, 1 H) 7.14 (s, 1 H) 7.25 (d, J = 7.91 Hz, 1 H) 7.32-7.51 (m, 2 H) 7.50-7.84 (m, 5 H) 8.07 (dd, J = 6.78, 2.64 Hz, 1 H) 8.70 (s, 1 H) 10.30 (s, 1 H) 12.46 (s, 1 H) | (E)-3-(3- boronophenyl) acrylic acid and 5-bromo-N- (3-chloro-4- fluorophenyl)- 4-(1H- imidazol-1- yl)pyrimidin- 2-amine (Intermediate 118) |

### Example 360

### 3-(2-(3-chloro-4-fluorophenylamino)-4-(3-(dimethylamino)propylamino)pyrimidin-5-yl)-N-methoxybenzamide

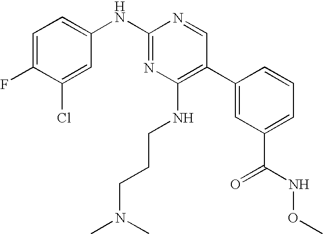

To a stirred solution of 3-(2-[(3-chloro-4-fluorophenyl)amino]-4-{[3-(dimethylamino)propyl]amino}pyrimidin-5-yl)benzoic acid (Example 31, 52 mg, 0.12 mmol) and triethylamine (0.057 ml, 0.4 mmol) in DMF (1.5 ml) under ambient conditions was added O-(7-azabenzotriazol-1-yl)-N,N,N′,N′-tetramethyluronium hexafluorophosphate (HATU; 44 mg, 0.12 mmol) as a solid. The mixture was stirred for 10 minutes; to it was then added methoxylamine hydrochloride (8 mg, 0.12 mmol) as a solid. The mixture was stirred until complete conversion was seen by LCMS. The reaction mixture was diluted with 1-3 mL water while stiffing continued; a light pink precipitate formed. Stirring continued for several minutes, and the vessel was then transferred to an ice bath for 10 minutes. 3-(2-(3-Chloro-4-fluorophenylamino)-4-(3-(dimethylamino)propylamino)pyrimidin-5-yl)-N-methoxybenzamide was collected (22 mg) and characterized by LCMS and ¹H NMR.

MS: ES+ 473 for C₂₃H₂₆ClFN₆O₂

¹H NMR (300 MHz, DMSO-D6) δ ppm 1.84-2.01 (m, 2H) 2.76 (d, J=4.71 Hz, 6H) 2.97-3.12 (m, 2H) 3.35-3.49 (m, 2H) 3.72 (s, 3H) 7.42 (t, J=9.04 Hz, 1H) 7.50-7.64 (m, 4H) 7.76-7.84 (m, 2H) 7.89 (s, 1H) 8.07 (dd, J=6.78, 2.64 Hz, 1H) 9.50 (s, 1H) 10.22 (s, 1H) 11.84 (s, 1H)

The following examples were prepared using the general HATU coupling method described above for Example 360 using the starting materials (SM) indicated.

| Ex | Compound | Data | SM |
|---|---|---|---|
| 361 | 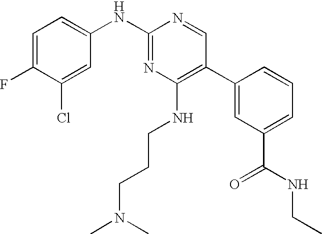 3-(2-(3-chloro-4- fluorophenylamino)-4- (3-(dimethylamino) propylamino) pyrimidin-5-yl)-N- ethylbenzamide | MS: ES+ 471 for C₂₄H₂₈ClFN₆O ¹H NMR (300 MHz, DMSO- D6) δ ppm 1.11 (t, J = 7.25 Hz, 3 H) 1.59-1.75 (m, 2 H) 1.94 (s, 6 H) 2.28 (t, J = 6.41 Hz, 2 H) 3.18-3.36 (m, 2 H) 3.37-3.53 (m, 2 H) 7.22-7.34 (m, 2 H) 7.44-7.68 (m, 3 H) 7.77-7.87 (m, 3 H) 8.26 (dd, J = 6.97, 2.64 Hz, 1 H) 8.50 (t, J = 5.46 Hz, 1 H) 9.39 (s, 1 H) | Ethylamine and 3-(2-[(3- chloro-4- fluorophenyl) amino]-4-{[3- (dimethyl- amino)propyl] amino} pyrimidin-5- yl)benzoic acid (Example 31) |
| 362 | 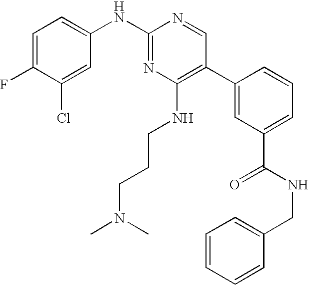 N-benzyl-3-(2-(3-chloro- 4-fluorophenylamino)-4- (3-(dimethylamino) propylamino) pyrimidin-5-yl) benzamide | MS: ES+ 533 for C₂₉H₃₀ClFN₆O ¹H NMR (300 MHz, DMSO- D6) δ ppm 1.56-1.75 (m, 2 H) 1.92 (s, 6 H) 2.27 (t, J = 6.31 Hz, 2 H) 3.37-3.51 (m, 2 H) 4.48 (d, J = 6.03 Hz, 2 H) 7.19-7.37 (m, 7 H) 7.48-7.58 (m, 2 H) 7.58-7.67 (m, 1 H) 7.82 (s, 1 H) 7.85-7.92 (m, 2 H) 8.26 (dd, J = 6.88, 2.54 Hz, 1 H) 9.09 (t, J = 6.03 Hz, 1 H) 9.39 (s, 1 H) | Benzylamine and 3-(2-[(3- chloro-4- fluorophenyl) amino]-4-{[3- (dimethyl- amino)propyl] amino} pyrimidin-5- yl)benzoic acid (Example 31) |
| 363 | 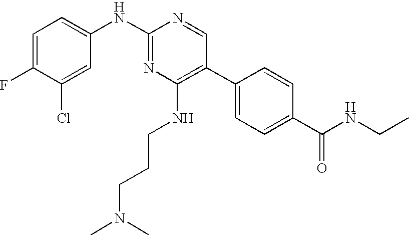 4-(2-(3-chloro-4- fluorophenylamino)-4- (3-(dimethylamino) propylamino) pyrimidin-5-yl)-N- ethylbenzamide | MS: ES+ 471 for C₂₄H₂₈ClFN₆O ¹H NMR (300 MHz, DMSO- D6) δ ppm 1.12 (t, J = 7.16 Hz, 3 H) 1.60-1.74 (m, 2 H) 1.98 (s, 6 H) 2.29 (t, J = 6.41 Hz, 2 H) 3.22-3.37 (m, 2 H) 3.43 (q, J = 6.22 Hz, 2 H) 7.22-7.34 (m, 2 H) 7.38-7.49 (m, 2 H) 7.57- 7.67 (m, 1 H) 7.80 (s, 1 H) 7.93 (d, J = 8.29 Hz, 2 H) 8.25 (dd, J = 6.78, 2.64 Hz, 1 H) 8.51 (t, J = 5.56 Hz, 1 H) 9.40 (s, 1 H) | ethylamine and 4-(2-[(3- chloro-4- fluorophenyl) amino]-4-{[3- (dimethyl- amino)propyl] amino} pyrimidin-5- yl)benzoic acid (Example 30) |
| 364 | 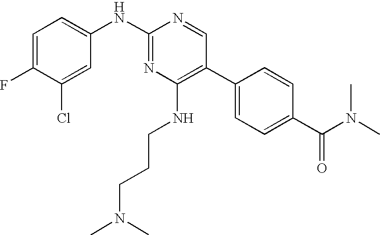 4-(2-(3-chloro-4- fluorophenylamino)-4- (3-(dimethylamino) propylamino) pyrimidin-5-yl)-N,N- dimethylbenzamide | MS: ES+ 471 for C₂₄H₂₈ClFN₆O ¹H NMR (300 MHz, DMSO- D6) δ ppm 1.91-2.07 (m, 2 H) 2.68 (s, 3 H) 2.75 (s, 6 H) 2.98- 3.02 (m, 2 H) 3.06 (s, 3 H) 3.44- 3.60 (m, 2 H) 7.00 (t, J = 5.46 Hz, 1 H) 7.39 (t, J = 9.14 Hz, 1 H) 7.48-7.59 (m, 3 H) 7.64- 7.73 (m, 1 H) 7.90 (s, 1 H) 8.28 (dd, J = 6.88, 2.54 Hz, 1 H) 9.50 (s, 1 H) 11.76 (s, 1 H) | Dimethylamine and 4-(2-[(3- chloro-4- fluorophenyl) amino]-4-{[3- (dimethyl- amino)propyl] amino} pyrimidin-5- yl)benzoic acid (Example 30) |
| 365 | 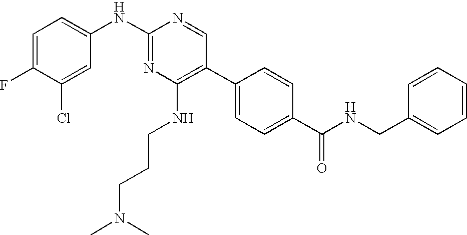 N-benzyl-4-(2-(3-chloro- 4-fluorophenylamino)-4- (3-(dimethylamino) propylamino) pyrimidin-5-yl) benzamide | MS: ES+ 533 for C₂₉H₃₀ClFN₆O ¹H NMR (300 MHz, DMSO- D6) δ ppm 1.62-1.73 (m, 2 H) 1.97 (s, 6 H) 2.28 (t, J = 6.31 Hz, 2 H) 3.38-3.50 (m, 2 H) 4.51 (d, J = 6.03 Hz, 2 H) 7.19-7.36 (m, 7 H) 7.47 (d, J = 8.29 Hz, 2 H) 7.57-7.67 (m, 1 H) 7.81 (s, 1 H) 8.00 (d, J = 8.48 Hz, 2 H) 8.25 (dd, J = 6.97, 2.64 Hz, 1 H) 9.11 (t, J = 6.03 Hz, 1 H) 9.41 (s, 1 H) | benzylamine and 4-(2-[(3- chloro-4- fluorophenyl) amino]-4-{[3- (dimethyl- amino)propyl] amino} pyrimidin-5- yl)benzoic acid (Example 30) |
| 366 | 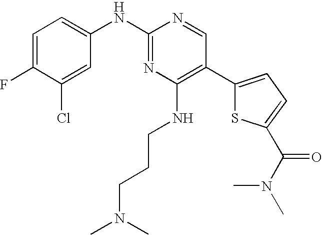 5-(2-(3-chloro-4- fluorophenylamino)-4- (3-(dimethylamino) propylamino) pyrimidin-5-yl)-N,N- dimethylthiophene-2- carboxamide | MS: ES+ 477 for C₂₂H₂₆ClFN₆OS ¹H NMR (300 MHz, DMSO- D6) δ 1.65-1.79 (m, 2 H) 2.05 (s, 6 H) 2.33 (t, J = 6.12 Hz, 2 H) 2.47-2.57 (m, 3 H) 3.34 (s, 3 H) 3.48 (q, J = 5.97 Hz, 2 H) 7.13 (d, J = 3.77 Hz, 1 H) 7.30 (t, J = 9.14 Hz, 1 H) 7.49-7.71 (m, 3 H) 7.95 (s, 1H) 8.23 (dd, J = 6.78, 2.26 Hz, 1 H) 9.53 (s, 1 H) | dimethylamine and 5-{2-(3- chloro-4- fluorophenyl amino)-4-[3- (dimethyl- amino) propylamino] pyrimidin-5- yl} thiophene-2- carboxylic acid (Example 17) |
| 367 | 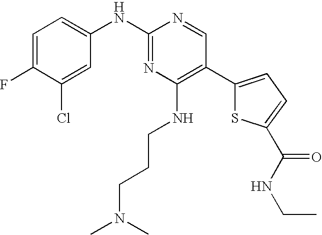 5-(2-(3-chloro-4- fluorophenylamino)-4- (3-(dimethylamino) propylamino) pyrimidin-5-yl)-N- ethylthiophene-2- carboxamide | MS: ES+ 477 for C₂₂H₂₆ClFN₆OS ¹H NMR (300 MHz, DMSO- D6) δ ppm 1.11 (t, J = 7.16 Hz, 3 H) 1.64-1.76 (m, 2 H) 2.03 (s, 6 H) 2.32 (t, J = 6.31 Hz, 2 H) 3.20-3.31 (m, 2 H) 3.46 (q, J = 6.03 Hz, 2 H) 7.12 (d, J = 3.77 Hz, 1 H) 7.23-7.35 (m, 1 H) 7.45-7.66 (m, 2 H) 7.74 (d, J = 3.77 Hz, 1 H) 7.92 (s, 1 H) 8.21 (dd, J = 6.97, 2.64 Hz, 1 H) 8.50 (t, J = 5.56 Hz, 1 H) 9.51 (s, 1 H) | ethylamine and 5-{2-(3- chloro-4- fluorophenyl amino)-4-[3- (dimethyl- amino) propylamino] pyrimidin-5- yl} thiophene-2- carboxylic acid (Example 17) |
| 368 | 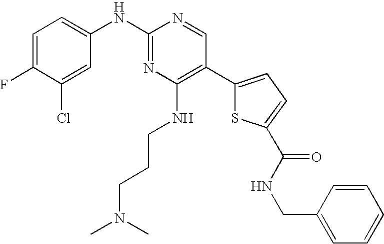 N-benzyl-5-(2-(3-chloro- 4-fluorophenylamino)-4- (3-(dimethylamino) propylamino) pyrimidin-5-yl) thiophene-2- carboxamide | MS: ES+ 539 for C₂₇H₂₈ClFN₆OS ¹H NMR (300 MHz, DMSO- D6) δ ppm 1.61-1.77 (m, 2 H) 2.03 (s, 6 H) 2.31 (t, J = 6.22 Hz, 2 H) 3.45 (q, J = 5.90 Hz, 2 H) 4.46 (d, J = 5.84 Hz, 2 H) 7.15 (d, J = 3.77 Hz, 1 H) 7.19-7.39 (m, 6 H) 7.44-7.66 (m, 2 H) 7.83 (d, J = 3.96 Hz, 1 H) 7.93 (s, 1 H) 8.22 (dd, J = 6.97, 2.45 Hz, 1 H) 9.08 (t, J = 6.03 Hz, 1 H) 9.51 (s, 1 H) | benzylamine and 5-{2-(3- chloro-4- fluorophenyl amino)-4-[3- (dimethyl- amino) propylamino] pyrimidin-5- yl} thiophene-2- carboxylic acid (Example 17) |
| 369 | 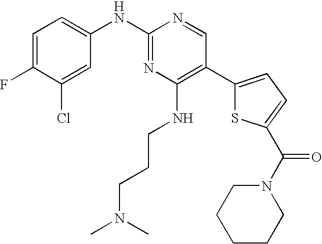 (5-(2-(3-chloro-4- fluorophenylamino)-4- (3-(dimethylamino) propylamino) pyrimidin-5-yl) thiophen-2-yl) (piperidin-1-yl) methanone | MS: ES+ 517 for C₂₅H₃₀ClFN₆OS ¹H NMR (300 MHz, DMSO- D6) δ ppm 1.46-1.76 (m, 8 H) 2.04 (s, 6 H) 2.32 (t, J = 6.31 Hz, 2 H) 3.41-3.51 (m, 2 H) 3.57- 3.68 (m, 4 H) 7.10 (d, J = 3.58 Hz, 1 H) 7.29 (t, J = 9.14 Hz, 1 H) 7.39 (d, J = 3.77 Hz, 1 H) 7.53 (t, J = 4.80 Hz, 1 H) 7.57- 7.66 (m, 1 H) 7.93 (s, 1 H) 8.22 (dd, J = 6.88, 2.54 Hz, 1 H) 9.51 (s, 1 H) | piperidine and 5-{2-(3- chloro-4- fluorophenyl amino)-4-[3- (dimethyl- amino) propylamino] pyrimidin-5- yl} thiophene-2- carboxylic acid (Example 17) |
| 370 | 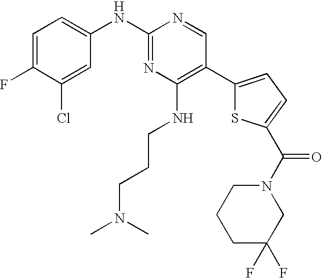 (5-(2-(3-chloro-4- fluorophenylamino)-4- (3-(dimethylamino) propylamino) pyrimidin-5-yl)thiophen- 2-yl) (3,3-difluoro- piperidin-1-yl) methanone | MS: ES+ 553 for C₂₅H₂₈ClF₃N₆OS ¹H NMR (300 MHz, DMSO-d₆) δ ppm 1.65-1.80 (m, 4 H) 2.04 (s, 6 H) 2.08-2.20 (m, 2 H) 2.32 (t, J = 6.31 Hz, 2 H) 3.46 (q, J = 6.03 Hz, 2 H) 3.70 (br. s., 2 H) 3.99 (t, J = 11.87 Hz, 2 H) 7.14 (d, J= 3.77 Hz, 1 H) 7.29 (t, J = 9.14 Hz, 1 H) 7.48 (d, J = 3.77 Hz, 1 H) 7.52-7.66 (m, 2 H) 7.95 (s, 1 H) 8.21 (dd, J = 6.97, 2.64 Hz, 1 H) 9.53 (s, 1 H) | 3,3- difluoro- piperidine and 5-{2-(3- chloro-4- fluorophenyl amino)-4-[3- (dimethyl- amino) propylamino] pyrimidin-5- yl} thiophene-2- carboxylic acid (Example 17) |
| 371 | 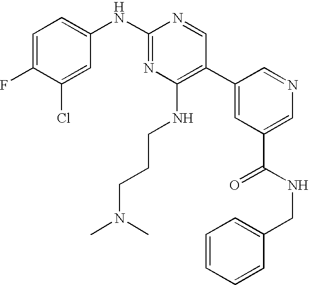 N-benzyl-5-(2-(3- chloro-4- fluorophenylamino)-4- (3-(dimethylamino) propylamino) pyrimidin-5-yl) nicotinamide | MS: ES+ 534 for C₂₈H₂₉ClFN₇O ¹H NMR (300 MHz, DMSO- D6) δ ppm 1.85-2.00 (m, 2 H) 2.73 (d, J = 4.71 Hz, 6 H) 2.98- 3.09 (m, 2 H) 3.33-3.47 (m, 2 H) 4.53 (d, J = 5.84 Hz, 2 H) 7.15-7.62 (m, 7 H) 7.92-8.39 (m, 4 H) 8.77 (d, J = 1.88 Hz, 1 H) 9.13 (d, J = 1.88 Hz, 1 H) 9.34 (t, J = 5.93 Hz, 1 H) 9.85 (s, 1 H) 10.82 (s, 1 H) | benzylamine and 5-(2-(3- chloro-4- fluorophenyl amino)-4-(3- dimethyl- amino)propyl- amino) pyrimidin- 5- yl)nicotinic acid (Example 214) |
| 372 | 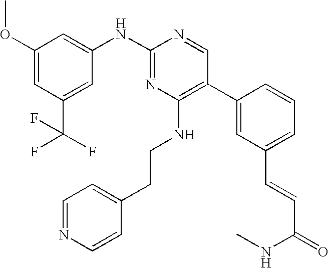 (E)-3-(3-(2-(3- methoxy-5- (trifluoromethyl)phenyl amino)-4-(2-pyridin-4- yl) ethylamino)pyrimidin- 5-yl) phenyl)-N-methyl acrylamide | MS: ES+ 549 for C₂₉H₂₇F₃N₆O₂ ¹H NMR (300 MHz, DMSO-d₆) δ ppm 2.71 (d, 3 H) 3.12 (t, J = 6.69 Hz, 2 H) 3.72 (q, J = 6.41 Hz, 2 H) 3.82 (s, 3 H) 6.63 (d, 1 H) 6.98 (s, 1 H) 7.27-7.55 (m, 5 H) 7.63 (d, J = 7.91 Hz, 1 H) 7.70-7.87 (m, 4 H) 7.92 (s, 1 H) 8.07-8.16 (m, 1 H) 8.75 (d, 2 H) 10.55 (br. s., 1 H) | methylamine and (E)-3-(3-(2- (3-methoxy- 5- (trifluoro- methyl)phenyl amino)-4-(2- (pyridin-4-yl) ethylamino) pyrimidin-5- yl)phenyl) acrylic acid (Example 355) |
| 373 | 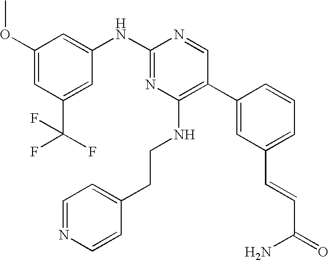 (E)-3-(3-(2-(3- methoxy-5- (trifluoromethyl)phenyl amino)-4-(2-(pyridin-4- yl) ethylamino)pyrimidin- 5-yl) phenyl)acrylamide | MS: ES+ 535 for C₂₈H₂₅F₃N₆O₂ ¹H NMR (300 MHz, DMSO-d₆) δ ppm 3.10 (t, 2 H) 3.71 (q, J = 6.28 Hz, 2 H) 3.81 (s, 3 H) 6.63 (d, 1 H) 6.94 (s, 1 H) 7.10- 7.82 (m, 12 H) 7.90 (s, 1 H) 8.72 (d, J = 6.22 Hz, 2 H) 10.32 (br. s., 1 H) | ammonia (0.5 M) in dioxane and (E)-3-(3-(2- (3-methoxy- 5- (trifluoro- methyl)phenyl amino)-4-(2- (pyridin-4-yl) ethylamino) pyrimidin-5- yl)phenyl) acrylic acid (Example 355) |
| 374 | 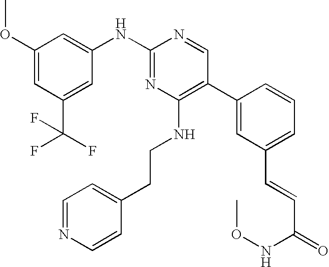 (E)-N-methoxy-3-(3- (2-(3-methoxy-5- (trifluoromethyl)phenyl amino)-4-(2-(pyridin-4- yl) ethylamino)pyrimidin- 5-yl)phenyl)acrylamide | MS: ES+ 565 for C₂₉H₂₇F₃N₆O₃ ¹H NMR (300 MHz, DMSO-d₆) δ ppm 3.14 (t, 2 H) 3.67 (s, 3 H) 3.73 (q, J = 6.22 Hz, 2 H) 3.82 (s, 3 H) 7.00 (s, 1 H) 7.34 (d, J = 7.54 Hz, 1 H) 7.45-7.57 (m, 5 H) 7.61-7.74 (m, 3 H) 7.79 (d, J = 6.22 Hz, 2 H) 7.95 (s, 2 H) 8.77 (d, J = 6.22 Hz, 2 H) 10.81 (br. s., 1 H) | methoxyl amine hydrochloride and (E)-3-(3-(2- (3-methoxy- 5- (trifluoro- methyl)phenyl amino)-4-(2- (pyridin-4-yl) ethylamino) pyrimidin-5- yl)phenyl) acrylic acid (Example 355) |
| 375 | 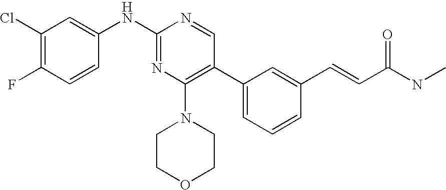 (E)-3-(3-(2-(3-chloro- 4-fluorophenylamino)- 4- morpholinopyrimidin- 5-yl)phenyl)-1H- methylacrylamide | MS(ES): 468 (M + 1) for C₂₄H₂₃ClFN₅O₂ ¹H NMR (300 MHz, DMSO- d6) d ppm 2.70 (d, J = 4.52 Hz, 3 H) 3.23 (m, 4 H) 3.54 (m, 4 H) 6.65 (d, J = 15.82 Hz, 1 H) 7.32 (t, J = 9.04 Hz, 1 H) 7.38-7.57 (m, 4 H) 7.57-7.75 (m, 2 H) 7.94-8.11 (m, 2 H) 8.14 (dd, J = 6.97, 2.64 Hz, 1 H) 9.63 (s, 1 H) | Methylamine and (E)-3-(3-(2- (3-chloro-4- fluorophenyl amino)-4- morpholino- pyrimidin-5- yl)phenyl) acrylic acid (Example 290) |
| 376 | 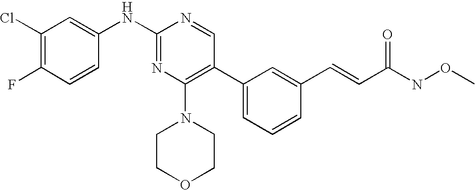 (E)-3-(3-(2-(3-chloro- 4-fluorophenylamino)- 4- morpholinopyrimidin- 5-yl)phenyl)-N- methoxyacrylamide | MS(ES): 484 (M + 1) for C₂₄H₂₃ClFN₅O₃ ¹H NMR (300 MHz, DMSO- d6) d ppm 3.23 (d, J = 3.39 Hz, 4 H) 3.54 (d, J = 3.96 Hz, 4 H) 3.66 (s, 3 H) 6.47 (d, J = 15.26 Hz, 1 H) 7.32 (t, J = 9.14 Hz, 1 H) 7.41- 7.77 (m, 6 H) 8.07 (s, 1 H) 8.14 (dd, J = 6.78, 2.45 Hz, 1 H) 9.63 (s, 1 H) 11.31 (s, 1 H) | Methoxyl- amine hydrochloride and (E)-3-(3-(2- (3-chloro-4- fluorophenyl amino)-4- morpholino- pyrimidin-5- yl)phenyl) acrylic acid (Example 290) |

### Example 377

### N-(3-(2-(3-chloro-4-fluorophenylamino)-5,5′-bipyrimidin-4-ylamino)propyl)acetamide

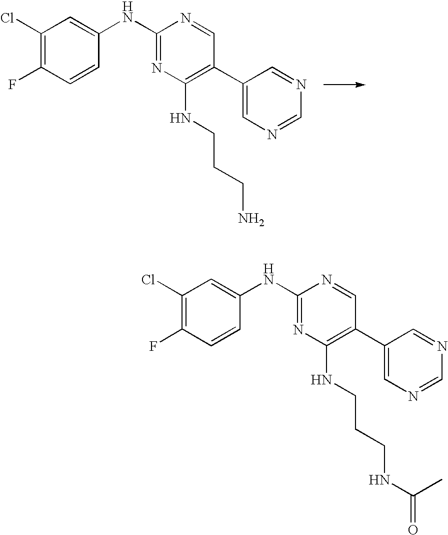

A solution of acetic anhydride (0.012 ml, 0.13 mmol) was added to N′-(3-aminopropyl)-N-(3-chloro-4-fluorophenyl)-5-pyrimidin-5-ylpyrimidine-2,4-diamine hydrochloride (Example 211, 53 mg, 0.13 mmol), triethylamine (0.054 ml, 0.39 mmol) and methylene chloride (1.5 ml) under nitrogen. The resultant mixture was stirred for 1 h, then concentrated under vacuum. The residue was chromatographed using 1-10% methanol/methylene chloride to yield the title compound (38 mg).

MS(ES): 416 (M+1) for C₁₉H₁₉ClFN₇O

¹H NMR (300 MHz, DMSO-d6) δ ppm 1.53-1.73 (m, 2H) 1.75 (s, 3H) 3.05 (q, J=6.03 Hz, 2H) 3.21-3.49 (m, 2H) 7.41 (t, J=9.04 Hz, 1H) 7.49-7.66 (m, 1H) 7.68-7.98 (m, 3H) 8.06 (dd, J=6.78, 2.64 Hz, 1H) 8.83 (s, 2H) 9.23 (s, 1H) 10.05 (s, 1H).

The following examples were prepared by the general method described above for Example 377 using the starting materials (SM) indicated.

| Ex | Compound | Data | SM |
|---|---|---|---|
| 378 | 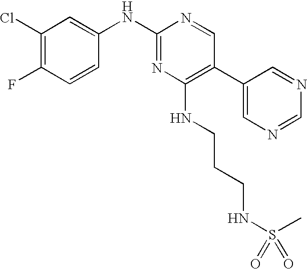 N-(3-(2-(3-chloro-4- fluorophenylamino)- 5,5′-bipyrimidin-4- ylamino)propyl) methanesulfonamide | MS(ES): 452 (M + 1) for C₁₈H₁₉ClFN₇O₂S ¹H NMR (300 MHz, DMSO-d6) δ ppm 1.61-1.94 (m, 2 H) 2.86 (s, 3 H) 2.99 (q, J = 6.47 Hz, 2 H) 3.37-3.50 (m, 2 H) 6.96 (t, J = 5.84 Hz, 1 H) 7.00-7.19 (m, 1 H) 7.33 (t, J = 9.14 Hz, 1 H) 7.52- 7.77 (m, 1 H) 7.86 (s, 1 H) 8.18 (dd, J = 6.88, 2.17 Hz, 1 H) 8.81 (s, 2 H) 9.17 (s, 1 H) 9.55 (s, 1 H) | methanesulfonyl chloride and N⁴-(3- aminopropyl)- N²-(3- chloro-4- fluorophenyl)- 5,5′- bipyrimidine- 2,4-diamine (Example 211) |

### General Procedure for the Coupling of Anilines to Intermediate 137

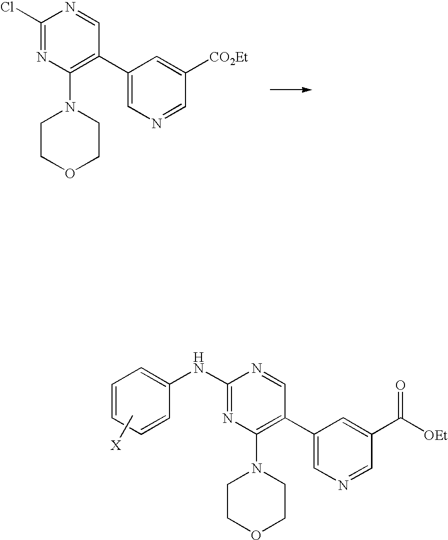

Intermediate 137 (1 eq) was suspended in a suitable solvent (e.g. acetonitrile, dioxane, or ethanol) (20 vol) and treated with the corresponding aniline (1 eq). The reaction was then treated with 4 N HCl (5 vol) in dioxane and refluxed under nitrogen. The reaction was monitored by TLC and then cooled to room temperature, diluted with diethyl ether and the solid was filtered and dried to afford the product. The compounds in the below table were prepared using this method with the specified starting material and solvent.

| Compound | Structure | Mass spectrum and ¹H NMR | SM |
|---|---|---|---|
| 6-[2-(3,5-Difluoro- 4-methoxy- phenylamino)-4- morpholin-4-yl- pyrimidin-5-yl]- pyridine-2- carboxylic acid ethyl ester Example 379^{a} | 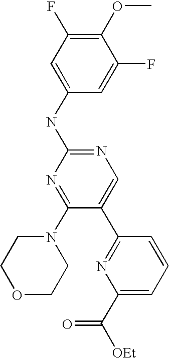 | MS(ES): 472 (M + 1) for C₂₃H₂₃F₂N₅O₄. 400 MHz, DMSO-d₆: δ 1.18 (t, J = 6.80 Hz, 3H), 3.22-3.23 (m, 4H), 3.56-3.58 (m, 4H), 3.85 (s, 3H), 4.38 (q, J = 7.20 Hz, 2H), 7.58 (d, J = 11.60 Hz, 2H), 8.20 (s, 1H), 8.40 (t, J = 2.00 Hz, 1H), 8.94 (d, J = 2.00 Hz, 1H), 9.02 (d, J = 1.60 Hz, 1H), 9.79 (s, 1H). | 3,5-Difluoro- 4-methoxy- phenylamine |
| 5-[2-(3-Methoxy-5- tetrazol-1-yl- phenylamino)-4- morpholin-4-yl- pyrimidin-5-yl]- nicotinic acid ethyl ester Example 380^{a} | 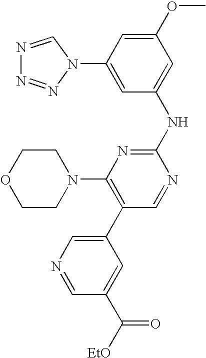 | MS(ES): 504.2 (M + 1) for C₂₄H₂₅N₉O₄. 400 MHz, DMSO-d₆ : δ 1.35 (t, J = 7.04 Hz, 3H), 3.26 (d, J = 3.80 Hz, 4H), 3.56 (br s, 4H), 3.84 (s, 3H), 4.37 (q, J = 6.96 Hz, 2H), 7.11 (d, J = 1.80 Hz, 1H), 7.46 (s, 1H), 8.21 (s, 1H), 8.28-8.31 (m, 1H), 8.39 (t, J = 2.04 Hz, 1H), 8.94 (d, J = 2.04 Hz, 1H), 9.01 (d, J = 1.92 Hz, 1H), 9.93 (s, 1H), 10.09 (s, 1H). | 3-Methoxy-5- tetrazol-1-yl- phenylamine Hydrochloride |
| 5-[2-(3-Cyano-5- fluoro- phenylamino)-4- morpholin-4-yl- pyrimidin-5-yl]- nicotinic acid ethyl ester Example 381^{b} | 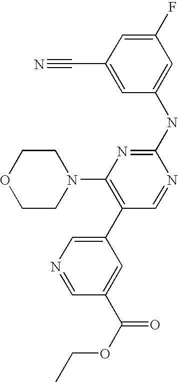 | MS(ES): 449 (M + 1) for C₂₃H₂₁FN₆O₃. 400 MHz, DMSO-d6: δ 1.34 (t, J = 7.04 Hz, 3H), 3.22-3.24 (m, 4H), 3.55-3.57 (m, 4H), 4.37 (q, J = 7.12 Hz, 2H), 7.36 (d, J = 9.60 Hz, 1H), 8.02 (d, J = 2.04 Hz, 1H), 8.04 (s, 1H), 8.24 (s, 1H), 8.39 (t, J = 2.12 Hz, 1H), 8.94 (d, J = 2.20 Hz, 1H), 9.02 (d, J = 2.00 Hz, 1H), 10.11 (s, 1H). | 3-Amino-5- fluoro- benzonitrile |
| 5-[2-(3-Chloro-5- cyano- phenylamino)-4- morpholin-4-yl- pyrimidin-5-yl]- nicotinic acid ethyl ester Example 382^{c} | 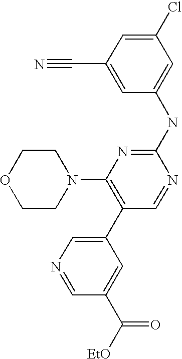 | MS(ES): 465.2 (M + 1) for C₂₃H₂₁ClN₆O₃. 400 MHz, DMSO-d₆: δ 1.35 (t, J = 7.00 Hz, 3H), 3.25 (d, J = 3.96 Hz, 4H), 3.50 (t, J = 5.08 Hz, 4H), 4.38 (q, J = 7.08 Hz, 2H), 7.66 (d, J = 1.16 Hz, 1H), 8.10 (d, J = 1.12 Hz, 1H), 8.18 (s, 1H), 8.23 (d, J = 8.32 Hz, 1H), 8.43 (s, 1H), 8.95 (d, J = 1.76 Hz, 1H), 9.07 (d, J = 1.48 Hz, 1H), 10.64 (s, 1H). | 3-Amino-5- chloro- benzonitrile |
| 6-[2-(3-Chloro-5- fluoro- phenylamino)-4- morpholin-4-yl- pyrimidin-5-yl]- pyridine-2- carboxylic acid ethyl ester Example 383^{a} | 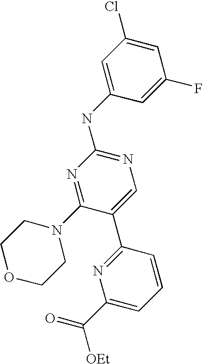 | MS(ES): 458.2 (M + 1) for C₂₂H₂₁ClFN₅O₃. 300 MHz, DMSO-d6: δ 1.34 (t, J = 7.11 Hz, 3H), 3.22 (br s, 4H), 3.56 (br s, 4H), 4.37 (q, J = 6.96 Hz, 2H), 6.92 (d, J = 8.46 Hz, 1H), 7.67 (d, J = 12.45 Hz, 1H), 7.77 (s, 1H), 8.21 (s, 1H), 8.39 (s, 1H), 8.94 (s, 1H), 9.01 (s, 1H), 9.95 (s, 1H). | 3-Chloro-5- fluoro- phenylamine |
| 5-[2-(3-Fluoro-5- methoxy- phenylamino)-4- morpholin-4-yl- pyrimidin-5-yl]- nicotinic acid ethyl ester Example 384^{a} | 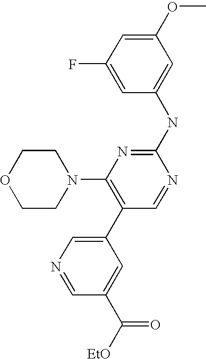 | MS(ES): 454 (M + 1) for C₂₃H₂₄FN₅O₄. 400 MHz, DMSO-d₆: δ 1.36 (t, J = 7.20 Hz, 3H), 3.40 (br s, 4H), 3.57 (br s, 4H), 3.78 (s, 3H), 4.39 (q, J = 7.20 Hz, 2H), 6.61 (dd, J = 2.00, 11.00 Hz, 1H), 7.10- 7.13 (m, 2H), 8.21 (s, 1H), 8.42 (t, J = 2.00 Hz, 1H), 8.94 (d, J = 2.00 Hz, 1H), 9.09 (d, J = 2.00 Hz, 1H), 10.79 (br s, 1H). | 3-Fluoro-5- methoxy- phenylamine |

Solvents used in the reaction
^{a}acetonitrile
^{b}ethanol
^{c}dioxane

### Example 385

### 5-[2-(3,5-Difluoro-phenylamino)-4-morpholin-4-yl-pyrimidin-5-yl]-nicotinic acid ethyl ester

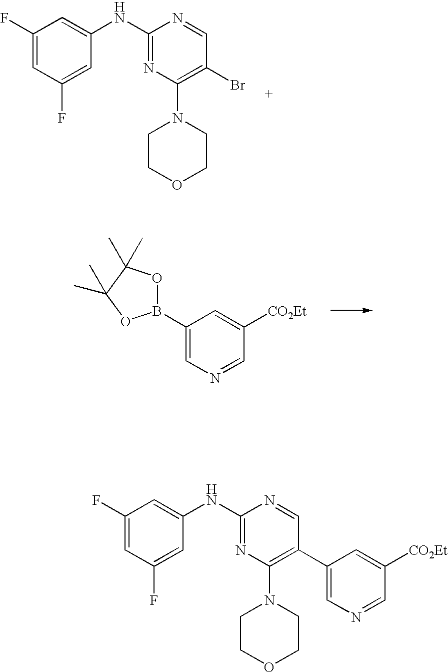

A suspension of Intermediate 140 (1.3 mmol, 0.5 g), 3-(ethoxycarbonyl)pyridine-5-boronic acid pinacolester (1.4 mmol, 0.4 g), tris(dibenzyledeneacetone)dipalladium(0) (0.4 mmol, 0.19 g), 2-dicyclohexylphosphino-2′,4′,6′-triisopropyl-1,1′-biphenyl (0.13 mmol, 0.12 g) and sodium carbonate (1.3 mmol, 0.146 g) in acetonitrile/water (10 mL:3 mL) was heated to 90° C. for 30 minutes. The reaction mixture was diluted with ethyl acetate (50 mL) and the organic layer was separated, dried over sodium sulfate, filtered and concentrated. The crude compound was purified by silica gel column chromatography using chloroform:methanol (9:1) as an eluent to yield Example 385 (0.2 g).

MS(ES): 442.2 (M+1) for C₂₂H₂₁F₂N₅O₃.

¹H-NMR (400 MHz, DMSO-d₆): δ 1.35 (q, J=7.08 Hz, 3H), 3.23 (d, J=3.76 Hz, 4H), 3.57 (d, J=3.64 Hz, 4H), 4.37 (q, J=7.12 Hz, 2H), 6.74 (t, J=2.24 Hz, 1H), 7.54 (d, J=10.12 Hz, 2H), 8.22 (d, J=2.84 Hz, 1H), 8.40 (t, J=1.96 Hz, 1H), 8.94 (d, J=2.04 Hz, 1H), 9.01 (d, J=1.80 Hz, 1H), 9.95 (s, 1H).

### Example 386

### 5-[2-(3,5-Dimethyl-phenylamino)-4-morpholin-4-yl-pyrimidin-5-yl]-nicotinic acid ethyl ester

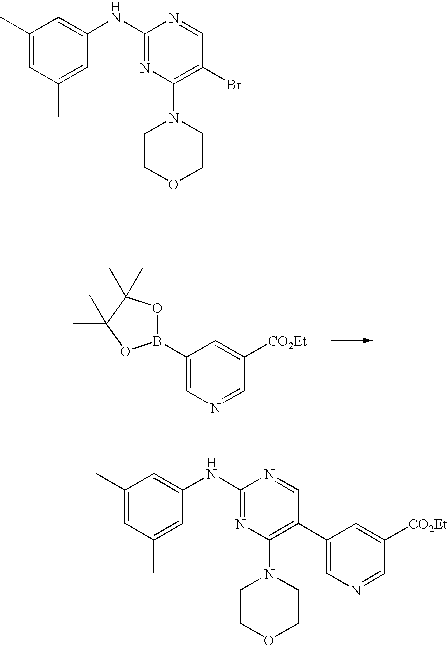

A suspension of Intermediate 144 (1.3 mmol, 0.5 g), 3-(ethoxycarbonyl)pyridine-5-boronic acid ester (1.4 mmol, 0.4 g), tris(dibenzyledeneacetone)dipalladium(0) (0.4 mmol, 0.19 g), 2-dicyclohexylphosphino-2′,4′,6′-triisopropyl-1,1′-biphenyl (0.13 mmol, 0.12 g) and sodium carbonate (1.3 mmol, 0.146 g) in acetonitrile/water (20 mL:5 mL) was heated to 90° C. for 30 minutes. The reaction mixture was diluted with ethyl acetate (50 mL) and the organic layer was separated, dried over sodium sulfate, filtered and concentrated. The crude compound was purified by silica gel column chromatography using chloroform:methanol (9:1) as an eluent to yield Example 386 (0.46 mmol, 0.2 g, 33%).

MS(ES): 433 (M) for C₂₄H₂₇N₅O₃.

¹H-NMR (400 MHz, DMSO-d₆): δ 1.34 (t, J=7.08 Hz, 3H), 2.22 (s, 6H), 3.21 (t, J=4.48 Hz, 4H), 3.56 (t, J=4.16 Hz, 4H), 4.37 (q, J=7.08 Hz, 2H), 6.58 (s, 1H), 7.40 (s, 2H), 8.15 (s, 1H), 8.38 (t, J=2.12 Hz, 1H), 8.93 (d, J=2.2 Hz, 1H), 8.98 (d, J=1.96 Hz, 1H), 9.39 (s, 1H).

### General procedure for the synthesis of 5-(4-Morpholin-4-yl-2-arylamino-pyrimidin-5-yl)-nicotinic acids from the corresponding esters

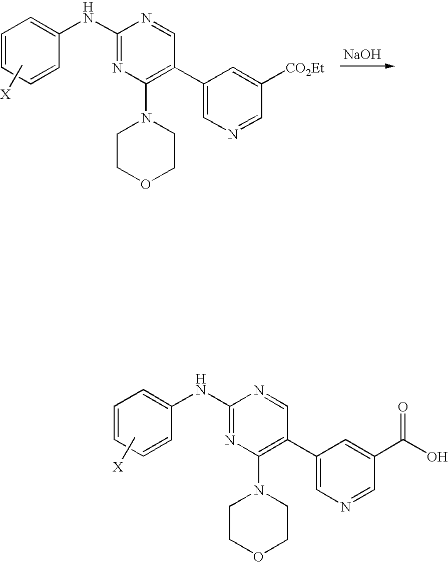

To a solution of carboxylic acid ester derivative (0.46 mmol) in tetrahydrofuran (5 mL) and water (5 mL), 1 N aq. sodium hydroxide (1.84 mmol, 1.84 ml) was added. The reaction was allowed to stir at room temperature for 2 h. After completion of reaction, the reaction mixture was carefully acidified with 1 N HCl and the solid that precipitated was filtered, washed with water and dried under vacuo to yield the desired 5-(4-Morpholin-4-yl-2-arylamino-pyrimidin-5-yl)-nicotinic acid. The compounds in the below table were prepared using this procedure and the specified starting material.

| Compound | Structure | Mass spectrum and ¹H NMR | SM |
|---|---|---|---|
| 5-[2-(3,5- Difluoro-4- methoxy- phenylamino)- 4-morpholin- 4-yl- pyrimidin-5- yl]-nicotinic acid Example 387 | 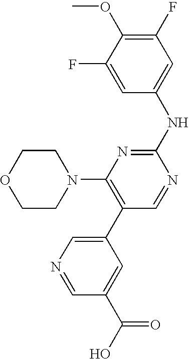 | MS(ES): 444 (M + 1) for C₂₁H₁₉F₂N₅O₄. 400 MHz, DMSO-d₆: δ 3.23 (br s, 4H), 3.55-3.56 (m, 4H), 3.90 (s, 3H), 7.57 (d, J = 11.20 Hz, 2H), 8.19 (s, 1H), 8.36 (s, 1H), 8.91 (d, J = 1.60 Hz, 1H), 9.01 (s, 1H), 9.85 (s, 1H), 13.70 (br s, 1H). | 6-[2-(3,5-Difluoro- 4-methoxy- phenylamino)-4- morpholin-4-yl- pyrimidin-5-yl]- pyridine-2- carboxylic acid ethyl ester Example 379 |
| 5-[2-(3,5- Difluoro- phenylamino)- 4-morpholin- 4-yl- pyrimidin-5- yl]nicotinic acid Example 388 | 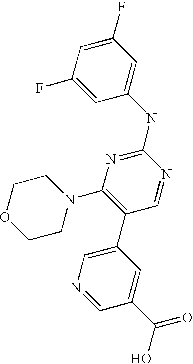 | MS(ES): 414 (M + 1) for C₂₀H₁₇F₂N₅O₃. 400 MHz, DMSO-d₆: δ 3.23 (d, J = 4.20 Hz, 4H), 3.56 (t, J = 4.56 Hz, 4H), 6.72 (tt, J = 2.28, 9.25 Hz, 1H), 7.54 (dd, J = 1.96, 10.44 Hz, 2H), 8.20 (s, 1H), 8.35 (t, J = 2.08 Hz, 1H), 8.91 (d, J = 2.16 Hz, 1H), 9.00 (d, J = 1.92 Hz, 1H), 9.94 (s, 1H), 13.54 (s, 1H). | 5-[2-(3,5-Difluoro- phenylamino)-4- morpholin-4-yl- pyrimidin-5-yl]- nicotinic acid ethyl ester Example 385 |
| 5-[2-(3,5- Dimethyl- phenylamino)- 4-morpholin- 4-yl- pyrimidin-5- yl]-nicotinic acid Example 389 | 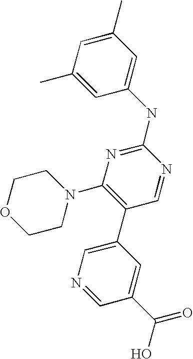 | MS(ES): 406 (M + 1) for C₂₂H₂₃N₅O₃. 400 MHz, DMSO-d₆: δ 2.22 (s, 6H), 3.21 (m, 4H), 3.56 (m, 4H), 6.58 (s, 1H), 7.4 (s, 2H), 8.13 (s, 1H), 8.33 (s, 1H), 8.89 (br s, 1H), 8.98 (br s, 1H), 9.37 (s, 1H), 13.52 (br s, 1H). | 5-[2-(3,5- Dimethyl- phenylamino)-4- morpholin-4-yl- pyrimidin-5- yl]- nicotinic acid ethyl ester Example 386 |
| 5-[2-(3- Methoxy-5- tetrazol-1-yl- phenyl- amino)-4- morpholin-4- yl-pyrimidin- 5-yl]- nicotinic acid Example 390 | 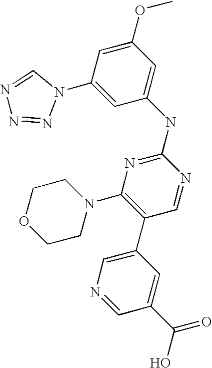 | MS(ES): 448 (M − 28) for C₂₂H₂₁N₉O₄. 400 MHz, DMSO-d₆ : δ 3.24 (br s, 4H), 3.56 (br s, 4H), 3.73 (s, 3H), 6.08 (s, 1H), 7.03 (s, 1H), 7.34 (s, 1H), 8.15 (s, 1H), 8.36 (s, 1H), 8.91 (s, 1H), 8.99 (s, 1H), 9.61 (s, 1H), 10.07 (s, 1H). | 5-[2-(3-Methoxy-5- tetrazol-1-yl- phenylamino-4- morpholin-4-yl- pyrimidin-5-yl]- nicotinic acid ethyl ester Example 380 |
| 5-[2-(3- Cyano-5- fluoro- phenylamino)- 4-morpholin- 4-yl- pyrimidin-5- yl]-nicotinic acid Example 391 | 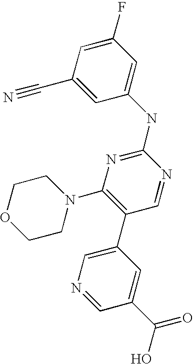 | MS(ES): 421 (M + 1) for C₂₁H₁₇FN₆O₃ 400 MHz, DMSO-d₆: δ 3.25 (br s, 4H), 3.57 (br s, 4H), 7.36 (d, J = 7.60 Hz, 1H), 8.03 (s, 1H), 8.05 (s, 1H), 8.23 (s, 1H), 8.36 (br s, 1H), 8.91 (s, 1H), 9.01 (s, 1H), 10.10 (s, 1H), 13.60 (br s, 1H). | 5-[2-(3-Cyano-5- fluoro- phenylamino)-4- morpholin-4-yl- pyrimidin-5-yl]- nicotinic acid ethyl ester Example 381 |
| 5-[2-(3- Chloro-5- cyano- phenylamino)- 4-morpholin- 4-yl- pyrimidin-5- yl]-nicotinic acid Example 392 | 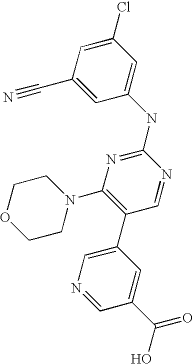 | MS(ES): 437 (M + 1) for C₂₁H₁₇ClN₆O₃. 400 MHz, DMSO-d₆: δ 3.24 (br s, 4H), 3.56 (br s, 4H), 7.53 (s, 1H), 8.16 (s, 1H), 8.22 (s, 1H), 8.25 (s, 1H), 8.34 (s, 1H), 8.90 (s, 1H), 9.00 (s, 1H), 10.10 (s, 1H), 13.60 (br s, 1H). | 5-[2-(3-chloro-5- cyano- phenylamino)-4- morpholin-4-yl- pyrimidin-5-yl]- nicotinic acid ethyl ester Example 382 |
| 6-[2-(3- Chloro-5- fluoro- phenylamino)- 4-morpholin- 4-yl- pyrimidin-5- yl]-pyridine- 2-carboxylic acid Example 393 | 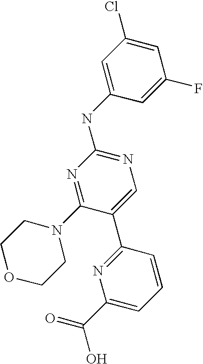 | MS(ES): 430 (M + 1) for C₂₀H₁₇ClFN₅O₃. 400 MHz, DMSO-d₆: δ 3.24-3.25 (m, 4H), 3.57 (br s, 4H), 6.93 (dd, J = 2.00, 8.40 Hz, 1H), 7.69 (d, J = 12.00 Hz, 1H), 7.78 (br s, 1H), 8.21 (br s, 1H), 8.36 (t, J = 2.00 Hz, 1H), 8.92 (br s, 1H), 9.01 (br s, 1H), 9.96 (br s, 1H), 13.60 (br s, 1H). | 6-[2-(3-Chloro-5- fluoro- phenylamino)-4- morpholin-4-yl- pyrimidin-5-yl]- pyridine-2- carboxylic acid ethyl ester Example 383 |
| 5-[2-(3- Fluoro-5- methoxy- phenylamino)- 4-morpholino- 4-yl- pyrimidin-5- yl]-nicotinic acid Example 394 | 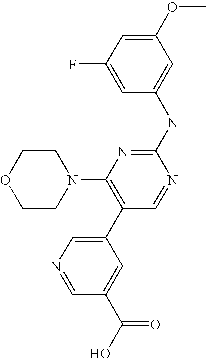 | MS(ES): 426 (M + 1) for C₂₁H₂₀FN₅O₄. 400 MHz, Acetic acid-d : δ 3.61 (br s, 4H), 3.76 (br s, 4H), 3.84 (s, 3H), 6.49 (d, J = 10.76 Hz, 1H), 7.10 (s, 1H), 7.21 (d, J = 10.4 Hz, 1H), 8.16 (s, 1H), 8.63 (s, 1H), 9.03 (br s, 1H), 9.28 (br s, 1H). | 5-[2-(3-Fluoro-5- methoxy- phenylamino)-4- morpholin-4-yl- pyrimidin-5-yl]- nicotinic acid ethyl ester Example 384 |

### General procedure for the coupling of anilines to Intermediate 145: {(E)-3-[3-(4-Morpholin-4-yl-2-arylamino-pyrimidin-5-yl)-phenyl]-acrylic acid ethyl ester}

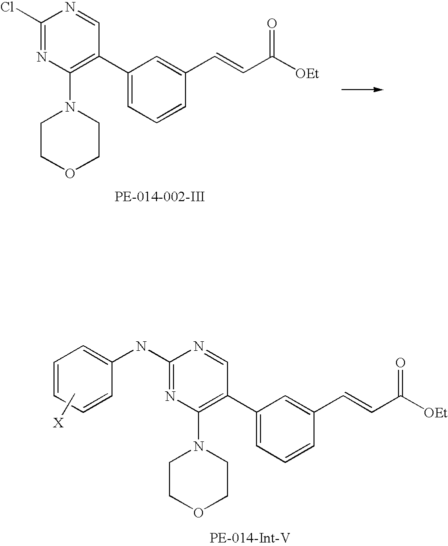

Intermediate 145 (1 eq) was suspended in acetonitrile/ethanol and treated with corresponding aniline (1 eq). The reaction was then treated with 4 N HCl (3 vol) in dioxane and refluxed under nitrogen for 5 hours. The mixture was cooled to room temperature, diluted with diethyl ether and the solid was filtered and dried to afford the product. The compounds in the below table were prepared using this procedure and the specified starting material and solvent.

| Compound | Structure | Mass spectrum and ¹H NMR | SM |
|---|---|---|---|
| (E)-3-{3-[2-(3,5- Difluoro-4- methoxy- phenylamino)-4- morpholin-4-yl- pyrimidin-5-yl]- phenyl}-acrylic acid ethyl ester Example 395^{d} | 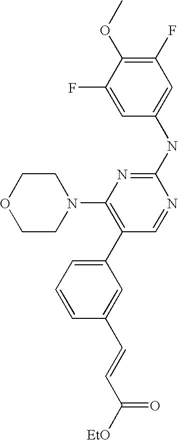 | MS(ES): 497 (M + 1) for C₂₆H₂₆F₂N₄O₄. 400 MHz, DMSO-d₆: δ 1.26 (t, J = 7.08 Hz, 3H), 3.23-3.24 (m, 4H), 3.54-3.56 (m, 4H), 3.83 (s, 3H), 4.20 (q, J = 7.12 Hz, 2H), 6.72 (d, J = 16.04 Hz, 1H), 7.46-7.66 (m, 4H), 7.68 (br s, 1H), 7.69 (d, J = 16.04 Hz, 1H), 7.82 (br s, 1H), 8.08 (br s, 1H), 9.66 (br s, 1H). | 3,5-Difluoro-4- methoxy- phenylamine |
| (E)-3-{3-[2-(3- Methoxy-5- tetrazol-1-yl- phenylamino)-4- morpholin-4-yl- pyrimidin-5-yl]- phenyl}-acrylic acid ethyl ester Example 396^{d} | 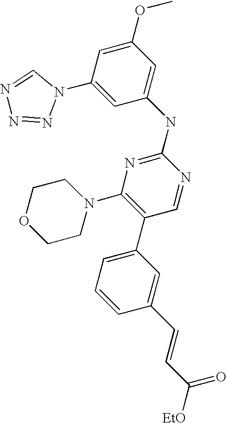 | MS(ES): 529 (M + 1) for C₂₇H₂₈N₈O₄. 300 MHz, DMSO-d₆: δ 1.25 (t, J = 6.00 Hz, 3H), 3.83 (s, 3H), 4.20 (q, 2H), 6.73 (d, J = 16.20 Hz, 1H), 7.09 (s, 1H), 7.46-7.56 (m, 3H), 7.67-7.72 (m, 2H), 7.83 (s, 1H), 8.12 (s, 1H), 8.28 (s, 1H), 9.82 (s, 1H), 10.08 (s, 1H). | 3-Methoxy-5- tetrazol-1-yl- phenylamine Hydrochloride |
| (E)-3-{3-[2-(3- Cyano-5-fluoro- phenylamino)-4- morpholin-4-yl- pyrimidin-5-yl]- phenyl}-acrylic acid ethyl ester Example 397^{e} | 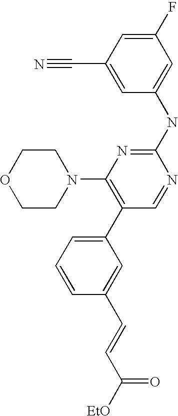 | MS(ES): 474 (M + 1) for C₂₆H₂₄FN₅O₃. 300 MHz, DMSO-d6: δ 1.25 (t, J = 7.02 Hz, 3H), 3.24-3.32 (m, 4H), 3.55 (br s, 4H), 4.19 (q, J = 7.02 Hz, 2H), 6.72 (d, J = 16.05 Hz, 1H), 7.31 (d, J = 7.89 Hz, 1H), 7.46-7.55 (m, 2H), 7.69 (d, J = 15.87 Hz, 1H), 7.69 (s, 1H), 7.82 (br s, 1H), 8.00 (m, 1H), 8.04 (br s, 1H), 8.13 (br s, 1H), 10.00 (br s, 1H). | 3-Amino-5- fluoro- benzonitrile |
| (E)-3-{3-[2-(3- Chloro-5-cyano- phenylamino)-4- morpholin-4-yl- pyrimidin-5-yl]- phenyl}-acrylic acid ethyl ester Example 398^{d} | 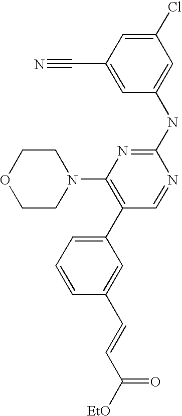 | MS(ES): 490.2 (M + 1) for C₂₆H₂₄ClN₅O₃. 400 MHz, DMSO-d₆: δ 1.27 (t, J = 7.20 Hz, 3H), 3.26 (s, 4H), 3.56 (d, J = 4.40 Hz, 4H), 4.20 (q, J = 7.20 Hz, 2H), 6.74 (d, J = 16.00 Hz, 1H), 7.50-7.54 (m, 3H), 7.71 (d, J = 16.00 Hz, 1H), 7.71 (s, 1H), 7.85 (s, 1H), 8.16-8.17 (m, 2H), 8.27 (t, J = 2.00 Hz, 1H), 10.01 (s, 1H). | 3-Amino-5- chloro- benzonitrile |
| (E)-3-{3-[2-(3- Chloro-5-fluoro- phenylamino)-4- morpholin-4-yl- pyrimidin-5-yl]- phenyl}-acrylic acid ethyl ester Example 399^{d} | 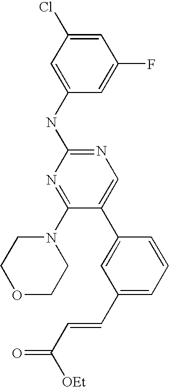 | MS(ES): 483 (M + 1) for C₂₅H₂₄ClFN₄O₃. 300 MHz, DMSO-d₆: δ 1.26 (t, J = 7.08 Hz, 3H), 3.24 (br s, 4H), 3.55 (br s, 4H), 4.19 (q, J = 7.11 Hz, 2H), 6.72 (d, J = 16.08 Hz, 1H), 6.90 (d, J = 8.25 Hz, 1H), 7.45- 7.55 (m, 2H), 7.66-7.72 (m, 3H), 7.77 (s, 1H), 7.83 (s, 1H), 8.11 (s, 1H), 9.84 (s, 1H). | 3-Chloro-5- fluoro- phenylamine |
| (E)-3-{3-[2-(3- Fluoro-5- methoxy- phenylamino)-4- morpholin-4-yl- pyrimidin-5-yl]- phenyl}-acrylic acid ethyl ester Example 400^{d} | 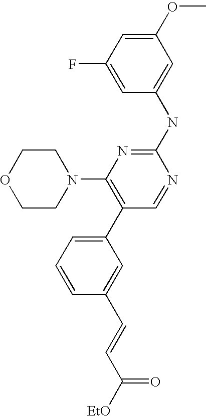 | MS(ES): 479 (M + 1) for C₂₆H₂₇FN₄O₄. 400 MHz, DMSO-d6: δ 1.26 (t, J = 7.04 Hz, 3H), 3.40 (br s, 4H), 3.56 (br s, 4H), 3.77 (s, 3H), 4.20 (q, J = 7.00 Hz, 2H), 6.57 (d, J = 10.84 Hz, 1H), 6.74 (d, J = 16.04 Hz, 1H), 7.10 (d, J = 1.36 Hz, 1H), 7.13 (br s, 1H), 7.49-7.54 (m, 2H), 7.68-7.75 (m, 2H), 7.83 (br s, 1H), 8.07 (br s, 1H), 10.52 (br s, 1H). | 3-Fluoro-5- methoxy- phenylamine |

Solvents used in the reaction
^{d}acetonitrile
^{e}ethanol

### Example 401

### (E)-3-{3-[2-(3,5-Difluoro-phenylamino)-4-morpholin-4-yl-pyrimidin-5-yl]-phenyl}-acrylic acid ethyl ester

### Example 402

### (E)-3-{3-[2-(3,5-Dimethyl-phenylamino)-4-morpholin-4-yl-pyrimidin-5-yl]-phenyl}-acrylic acid ethyl ester

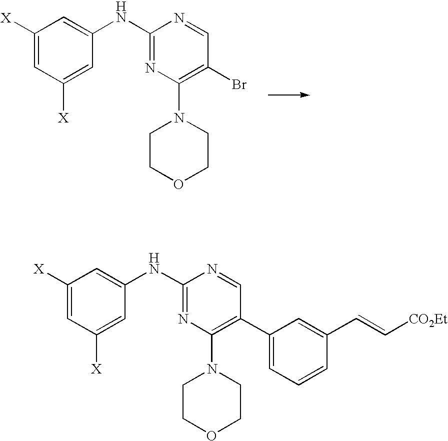

A suspension of the 5-bromopyrimidine derivative (1 eq), ethyl-3-borono cinnamate (1.1 eq), tris(dibenzyledeneacetone)dipalladium(0) (30 mol %), 2-dicyclohexylphosphino-2′,4′,6′-triisopropyl-1,1′-biphenyl (10 mol %) and sodium carbonate (1 eq) in acetonitrile/water (20:5 vol) was heated to 90° C. for 30 minutes. The reaction mixture was diluted with ethyl acetate (30 vol) and the organic layer was separated, dried over sodium sulfate, filtered and concentrated. The crude compound was purified by silica gel column chromatography using petroleum ether:EtOAc (7:3) as an eluent to yield the product. The compounds in the below table were prepared using this method and the specified starting material.

| Compound | Structure | Mass spectrum and ¹H NMR | SM |
|---|---|---|---|
| (E)-3-{3-[2-(3,5- Difluoro- phenylamino)-4- morpholin-4-yl- pyrimidin-5-yl]- phenyl}-acrylic acid ethyl ester Example 401 | 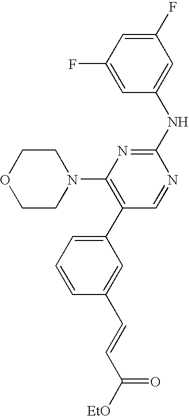 | MS(ES): 467.2 (M + 1) for C₂₅H₂₄F₂N₄O₃. 300 MHz, DMSO-d₆: δ 1.25 (t, J = 6.99 Hz, 3H), 3.23 (s, 4H), 3.55 (s, 4H), 4.19 (q, J = 7.02 Hz, 2H), 6.67-6.75 (m, 2H), 7.45-7.55 (m, 4H), 7.66-7.72 (m, 2H), 7.83 (s, 1H), 8.11 (s, 1H), 9.84 (s, 1H). | (5-Bromo-4- morpholin-4-yl- pyrimidin-2-yl)- (3,5-difluoro- phenyl)-amine Intermediate 140 |
| (E)-3-{33-[2-(3,5- Dimethyl- phenylamino)-4- morpholin-4-yl- pyrimidin-5-yl]- phenbyl}-acrylic acid ethyl ester Example 402 | 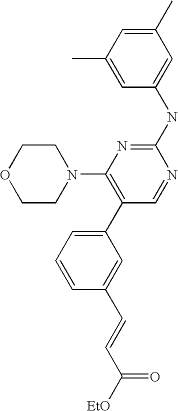 | MS(ES): 459 (M + 1) for C₂₇H₃₀N₄O₃. 400 MHz, DMSO-d6: δ 1.25 (t, J = 6.96 Hz, 3H), 2.22 (s, 6H), 3.23 (br s, 4H), 3.55 (br s, 4H), 4.19 (q, J = 6.76 Hz, 2H), 6.56 (s, 1H), 6.72 (d, J = 15.76 Hz, 1H), 7.40 (s, 2H), 7.44-7.54 (m, 2H), 7.64-7.71 (m, 2H), 7.81 (s, 1H), 8.05 (s, 1H), 9.28 (s, 1H). | (5-Bromo-4- morpholin-4-yl- pyrimidin-2-yl)- (3,5-dimethyl- phenyl)-amine Intermediate 144 |

### General procedure for the synthesis of (E)-3-[3-(4-Morpholin-4-yl-2-arylamino-pyrimidin-5-yl)-phenyl]-acrylic acid

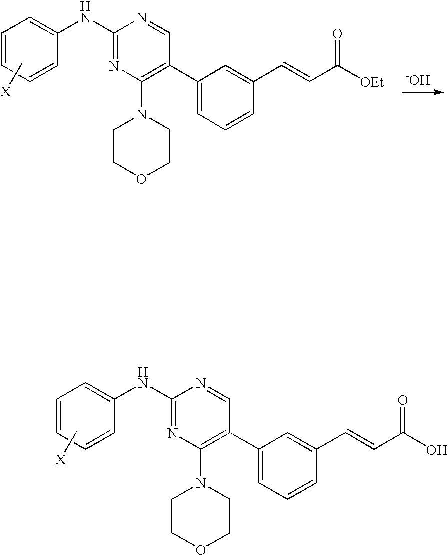

Ester compound (0.43 mmol, 0.2 g) was dissolved in tetrahydrofuran (5 mL), treated with 1 N aq. sodium hydroxide or Barium hydroxide (1.72 mmol) and was heated at 60° C. for 24 h. The reaction mixture was then carefully acidified with 1 N HCl and the solid that precipitated was filtered, washed with water and dried under vacuo to yield the product. The compounds in the below table were prepared using this procedure and the starting material and base specified.

| Compound | Structure | Mass spectrum and ¹H MR | SM |
|---|---|---|---|
| (E)-3-{3-[2- (3,5-Difluoro- 4-methoxy- phenylamino)- 4-morpholin-4- yl-pyrimidin-5- yl]-phenyl}- acrylic acid Example 403^{f} | 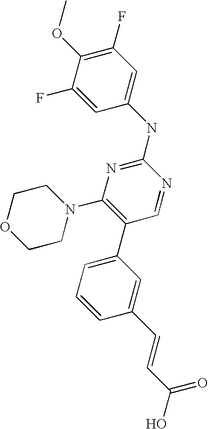 | MS(ES): 469 (M + 1) for C₂₄H₂₂F₂N₄O₄. 400 MHz, DMSO-d₆: δ 3.23-3.24 (m, 4H), 3.54-3.55 (m, 4H), 3.83 (s, 3H), 6.61 (d, J = 16.04 Hz, 1H), 7.46-7.56 (m, 2H), 7.59 (s, 1H), 7.63 (d, J = 15.84 Hz, 1H), 7.63 (s, 1H), 7.67 (s, 1H), 7.79 (s, 1H), 8.09 (s, 1H), 9.67 (br s, 1H), 12.44 (br s, 1H). | 3-{3-[2-(3,5- Difluoro-4- methoxy- phenylamino)-4- morpholin-4-yl- pyrimidin-5-yl]- phenyl}-acrylic acid ethyl ester Example 395 |
| (E)-3-{3-[2- (3,5-Difluoro- phenylamino)- 4-morpholin-4- yl-pyrimidin-5- yl]-phenyl}- acrylic acid Example 404^{f} | 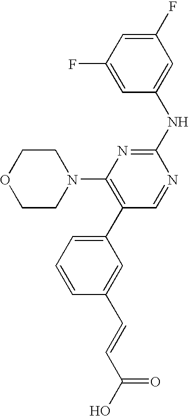 | MS(ES)P: 439 (M + 1) for C₂₃H₂₀F₂N₄O₃. 400 MHz, DMSO-d₆: δ 3.24 (d, J = 4.08 Hz, 4H), 3.55 (t, J = 4.32 Hz, 4H), 6.61 (d, J = 16.00 Hz, 1H), 6.70 (t, J = 9.12 Hz, 1H), 7.46-7.55 (m, 4H), 7.63 (d, J = 16.20 Hz, 1H), 7.63 (s, 1H), 7.79 (s, 1H), 8.11 (s, 1H), 9.84 (s, 1H). | 3-{3-[2-(3,5- Difluoro- phenylamino)-4- morpholin-4-yl- pyrimidin-5-yl]- phenyl}-acrylic acid ethyl ester Example 401 |
| (E)-3-{3-[2- (3,5-Dimethyl- phenylamino)- 4-morpholin-4- yl-pyrimidin-5- yl]-phenyl}- acrylic acid Example 405^{f} | 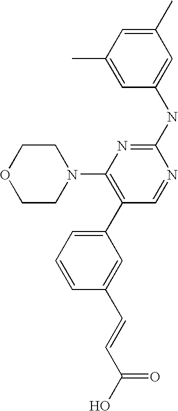 | MS(ES): 431 (M + 1) for C₂₅H₂₆N₄O₃. 400 MHz, DMSO-d₆: δ 2.22 (s, 6H), 3.23 (br s, 4H), 3.55 (br s, 4H), 6.56 (s, 1H), 6.59 (d, J = 16.0 Hz, 1H), 7.40 (s, 2H), 7.42-7.47 (m, 2H), 7.48-7.50 (m, 1H), 7.54- 7.58 (m, 1H), 7.73 (s, 1H), 8.04 (s, 1H), 9.26 (s, 1H). | 3-{3-[ 2-(3,5- Dimethyl- phenylamino)-4- morpholin-4-yl- pyrimidin-5-yl]- phenyl}-acrylic acid ethyl ester Example 402 |
| (E)-3-{3-[2-(3- Methoxy-5- tetrazol-1-yl- phenylamino)- 4-morpholin-4- yl-pyrimnidin-5- yl]-phenyl}- acrylic acid Example 406^{g} | 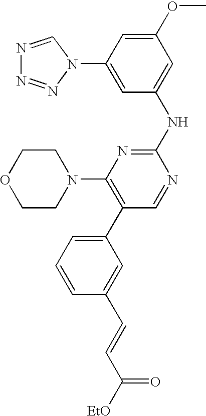 | MS(ES): 501 (M + 1) for C₂₅H₂₄N₈O₄ 400 MHz, DMSO-d₆: δ 3.27 (br s, 4H), 3.55 (br s, 4H), 3.84 (s, 3H), 6.61 (d, J = 16.04 Hz, 1H), 7.08 (br s,1H), 7.46-7.53 (m, 3H),m 7.60-7.64 (m, 2H), 7.79 (s, 1H), 8.11 (s, 1H), 8.28 (s, 1H), 9.82 (s, 1H), 10.18 (s, 1H), 12.44 (br s, 1H). | 3-{3-[2-(3- Methoxy-5- tetrazol-1-yl- phenylamino)-4- morpholin-4-yl- pyrimidin-5-yl]- phenyl}-acrylic acid ethyl ester Example 396 |
| (E)-3-{3-[2-(3- Cyano-5- fluoro- phenylamino)- 4-morpholin-4- yl-pyrimidin-5- yl]-phenyl}- acrylic acid Example 407^{f} | 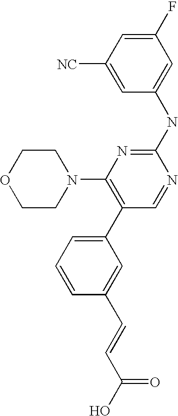 | MS(ES): 446 (M + 1) for C₂₄H₂₀FN₅O. 400 MHz, DMSO-d₆: δ 3.25-3.26 (m, 4H), 3.54-3.55 (m, 4H), 6.60 (d, J = 16.00 Hz, 1H), 7.32 (dd, J = 1.32, 8.12 Hz, 1H), 7.45-7.51 (m, 2H), 7.56 (d, J = 16.04 Hz, 1H), 7.62 (d, J = 7.08 Hz, 1H), 7.76 (br s, 1H), 8.01-8.05 (m, 2H), 8.13 (s, 1H), 10.03 (br s, 1H). | 3-{3-[2-(3-Cyano- 5-fluoro- phenylamino)-4- morpholin-4-yl- pyrimidin-5-yl]- phenyl}-acrylic acid ethyl ester Example 397 |
| (E)-3-{3-[2-(3- Chloro-5- cyano- phenylamino)- 4-morpholin-4- yl-pyrimidin-5- yl]-phenyl}- acrylic acid Example 408^{f} | 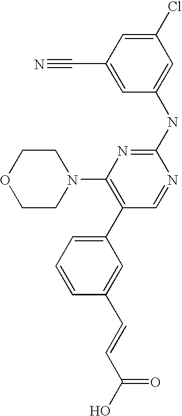 | MS(ES): 462 (M + 1) for C₂₄H₂₀ClN₅O₃. 400 MHz, DMSO-d₆ : δ 3.26 (d, J = 3.80 Hz, 4H), 3.56 (d, J = 3.80 Hz, 4H), 6.61 (d, J = 16.04 Hz, 1H), 7.46-7.53 (m, 3H), 7.61 (s, 1H), 7.65 (t, J = 3.56 Hz, 1H), 7.79 (s, 1H), 8.15 (t, J = 7.96 Hz, 2H), 8.26 (t, J = 1.84 Hz, 1H), 9.99 (s, 1H), 12.44 (br s, 1H). | 3-{3-[2-(3-Chloro- 5-cyano- phenylamino)-4- morpholin-4-yl- pyrimidin-5-yl]- phenyl}-acrylic acid ethyl ester Example 398 |
| (E)-3-{3-[2-(3- Chloro-5- fluoro- phenylamino)- 4-morpholin-4- yl-pyrimidin-5- yl]-phenyl}- acrylic acid Example 409^{f} | 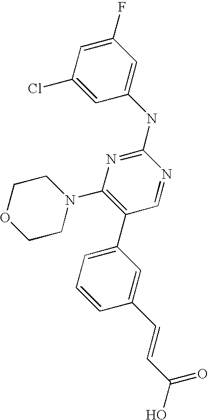 | MS(ES): 454 (M + 1) for C₂₃H₂₀ClFN₄O₃. 400 MHz, DMSO-d₆: δ 3.25 (br s, 4H), 3.55 (br s, 4H), 6.56 (d, J = 15.92 Hz, 1H), 6.90 (d, J = 8.08 Hz, 1H), 7.39-7.45 (m, 3H), 7.55 (m, 1H), 7.67-7.69 (m, 2H), 7.78 (m, 1H) 8.10 (s, 1H), 9.84 (s, 1H). | 3-{3- [2-(3-Chloro- 5-fluoro- phenylamino)-4- morpholin-4-yl- pyrimidin-5-yl]- phenyl}-acrylic acid ethyl ester Example 399 |
| (E)-3-{3-[2-(3- Fluoro-5- methoxy- phenylamino)- 4-morpholin-4- yl-pyrimidin-5- yl]-phenyl}- acrylic acid Example 410^{f} | 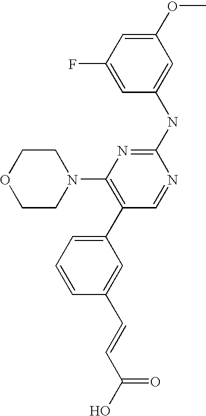 | MS(ES): 451 (M + 1) for C₂₄H₂₃FN₄O₄. 400 MHz, DMS)-d₆: δ 3.24 (br s, 4H), 3.55 (br s, 4H), 3.74 (s, 3H), 6.36 (dd, J = 2.04, 10.86 Hz, 1H), 6.61 (d, J = 16.04 Hz, 1H), 7.28- 7.32 (m, 2H), 7.46-7.54 (m, 2H), 7.61-7.65 (m, 2H), 7.79 (br s, 1H), 8.09 (br s, 1H), 9.59 (br s, 1H), 12.44 (br s, 1H). | 3-{3-[2-(3-Fluoro- 5-methoxy- phenylamino)-4- morpholin-4-yl- pyrimidin-5-yl]- phenyl}-acrylic acid ethyl ester Example 400 |

Base used in the reaction
^{f}NaOH
^{g}Ba(OH)₂

### Example 411

### 5-[2-(4-Fluoro-3-nitro-phenylamino)-4-morpholin-4-yl-pyrimidin-5-yl]-nicotinic acid ethyl ester

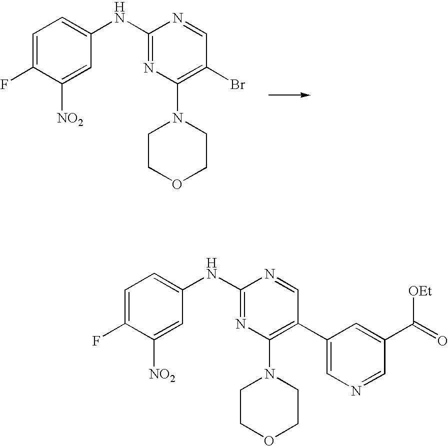

A suspension of 5-bromo-N-(4-fluoro-3-nitrophenyl)-4-morpholin-4-ylpyrimidin-2-amine Intermediate 150 (0.87 mmol, 0.35 g), (4,4,5,5-Tetramethyl-[1,3,2]dioxaborolan-2-yl)-nicotinic acid ethyl ester (0.92 mmol, 0.25 g), tris(dibenzylideneacetone)dipalladium(0) (10 mol %, 0.087 mmol, 80 mg), XPHOS (30 mol %, 0.26 mmol, 125 mg) and sodium carbonate (0.87 mmol, 92 mg) in acetonitrile/water (20 mL:5 mL) was degassed and heated to 90° C. for 30 min under inert atmosphere. The solvent was removed in vacuo and the crude mixture was taken in EtOAc (30 mL). It was then washed with water and brine. The organic layer was separated, dried over sodium sulfate, filtered and concentrated. The crude material was purified by silica gel column chromatography. The product eluted with CHCl₃:MeOH (98:2) eluent mixture. The title compound was obtained (0.2 g).

MS (ES): 469 (M+1) for C₂₂H₂₁FN₆O₅.

¹H-NMR (400 MHz, DMSO-d₆): δ 1.35 (t, J=7.04 Hz, 3H), 3.24-3.26 (m, 4H), 3.55-3.58 (m, 4H), 4.37 (q, J=7.08 Hz, 2H), 7.52 (dd, J=9.16, 11.14 Hz, 1H), 7.90 (dt, J=3.20, 6.10 Hz, 1H), 8.21 (s, 1H), 8.39 (t, J=2.08 Hz, 1H), 8.94 (d, J=2.24 Hz, 1H), 8.95-8.96 (m, 1H), 9.01 (d, J=1.92 Hz, 1H), 10.04 (br s, 1H).

### Example 412

### 3-{3-[2-(4-Fluoro-3-nitro-phenylamino)-4-morpholin-4-yl-pyrimidin-5-yl]-phenyl}-acrylic acid ethyl ester

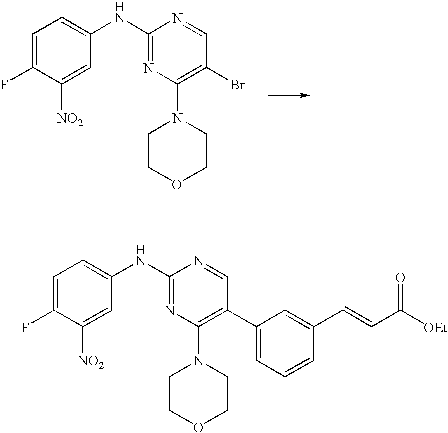

A suspension of 5-bromo-N-(4-fluoro-3-nitrophenyl)-4-morpholin-4-ylpyrimidin-2-amine Intermediate 150 (0.87 mmol, 0.35 g), ethyl boronocinnamate (0.92 mmol, 0.203 g), tris(dibenzylideneacetone)dipalladium(0) (10 mol %, 0.087 mmol, 80 mg), XPHOS (30 mol %, 0.26 mmol, 125 mg) and sodium carbonate (0.87 mmol, 92 mg) in acetonitrile/water (20 mL:5 mL) was degassed and heated to 90° C. for 30 min under inert atmosphere. The solvent was removed in vacuo and the crude mixture was taken in EtOAc (30 mL). It was then washed with water and brine. The organic layer was separated, dried over sodium sulfate, filtered and concentrated. The crude material was purified by silica gel column chromatography. The product eluted with Hexane:Ethyl Acetate (90:10) eluent mixture. The title compound was obtained (0.2 g).

MS (ES): 494 (M+1) for C₂₅H₂₄FN₅O₅.

¹H-NMR (400 MHz, DMSO-d₆): δ 1.25 (t, J=7.08 Hz, 3H), 3.27-3.28 (m, 4H), 3.55-3.57 (m, 4H), 4.19 (q, J=7.08 Hz, 2H), 6.73 (d, J=16.04 Hz, 1H), 7.46-7.55 (m, 3H), 7.67 (d, J=7.36 Hz, 1H), 7.69 (d, J=16.08 Hz, 1H), 7.83 (s, 1H), 7.88-7.91 (m, 1H), 8.11 (s, 1H), 8.96 (dd, J=2.80, 6.86 Hz, 1H), 9.94 (s, 1H).

### General procedure for the reaction of anilines with 5-(2-Chloro-4-morpholin-4-yl-pyrimidin-5-yl)-nicotinic acid ethyl ester (Intermediate 152)

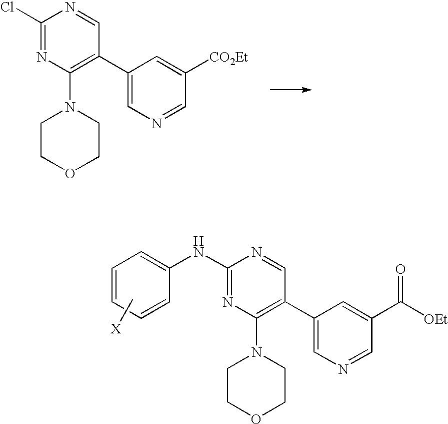

To a suspension of 5-(2-Chloro-4-morpholin-4-yl-pyrimidin-5-yl)-nicotinic acid ethyl ester Intermediate 152 (0.6 mmol, 1 eq.) taken in n-BuOH/acetonitrile/dioxane (10 mL) was added the corresponding aniline. The reaction mixture was then treated with 4 N HCl in dioxane (2 mL) and refluxed at 100° C. for 1.5 h. The reaction mixture was cooled to room temperature, diluted with diethyl ether, the solid filtered and dried to yield the corresponding nicotinic acid ethyl ester. The compounds in the table below were prepared using this general procedure and the starting material and solvent specified.

| Compound | Structure | Mass spectrum and ¹H NMR | SM |
|---|---|---|---|
| Example 413^{a} | 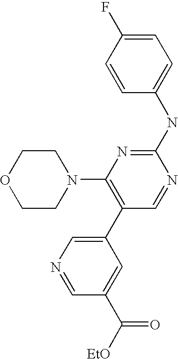 | MS(ES): 424 (M + 1) for C₂₂H₂₂FN₅O₃. 400 MHz, DMSO-d₆: δ 1.36 (t, J = 7.20 Hz, 3H), 3.33 (br s, 4H), 3.54 (br s, 4H), 4.39 (q, J = 7.20 Hz, 2H), 7.23 (t, J = 8.80 Hz, 2H), 7.63-7.66 (m, 2H), 8.14 (s, 1H), 8.38 (t, J = 2.00 Hz, 1H), 8.92 (d, J = 2.00 Hz, 1H), 9.07 (d, J = 2.00 Hz, 1H), 10.20 (s, 1H). | 4-fluoro aniline |
| Example 414^{b} | 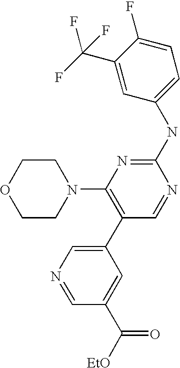 | MS(ES): 492 (M + 1) for C₂₃H₂₁F₄N₅O₃. 300 MHz, DMSO-d₆: δ 1.35 (t, J = 7.05 Hz, 3H), 3.35 (br s, 4H), 3.54 (br s, 4H), 4.38 (q, J = 6.99 Hz, 2H), 7.54 (t, J = 9.48 Hz, 1H), 7.84 (br s, 1H), 8.20 (s, 1H), 8.23 (d, J = 6.04 Hz, 1H), 8.40 (br s, 1H), 8.92 (br s, 1H), 9.07 (br s, 1H), 10.79 (br s, 1H). | 4-fluoro-3- trifluoro- methyl- aniline |
| Example 415^{b} | 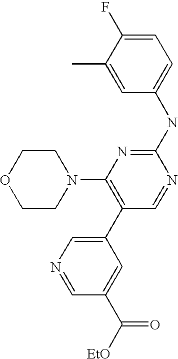 | MS(ES): 438 (M + 1) for C₂₃H₂₄FN₅O₃. 400 MHz, DMSO-d₆: δ 1.35 (t, J = 7.08 Hz, 3H), 2.23 (s, 3H), 3.32 (br s, 4H), 3.54-3.56 (m, 4H), 4.38 (q, J = 7.12 Hz, 2H), 7.14 (t, J = 9.12 Hz, 1H), 7.40-7.44 (m, 1H), 7.54 (dd, J = 2.44, 6.92 Hz, 1H), 8.11 (s, 1H), 8.36 (t, J = 2.12 Hz, 1H), 8.90 (d, J = 2.20 Hz, 1H), 9.05 (d, J = 1.96 Hz, 1H), 10.20 (br s, 1H). | 4-Fluoro-3- methyl- phenylamine |
| Example 416^{b} | 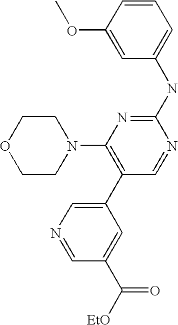 | MS(ES): 436 (M + 1) for C₂₃H₂₅N₅O₄. 300 MHz, DMSO-d₆: δ 1.34 (t, J = 7.05 Hz, 3H), 3.21 (br s, 4H), 3.55 (br s, 4H), 3.73 (s, 3H), 4.37 (q, J = 6.99 Hz, 2H), 6.51 (d, J = 8.82 Hz, 1H), 7.16 (t, J = 8.16 Hz,1H), 7.27 (d, J = 7.35 Hz,1H), 7.54 (s, 1H), 8.16 (s, 1H), 8.39 (s, 1H), 8.94 (s, 1H), 8.99 (s, 1H), 9.52 (s, 1H). | m-anisidine |
| Example 417^{b} | 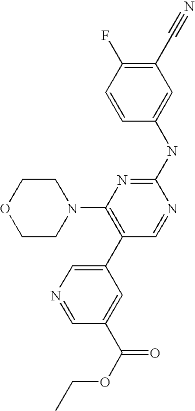 | MS(ES): 449 (M + 1) for C₂₃H₂₁FN₆O₃. 400 MHz, CDCl₃: δ 1.45 (t, J = 7.12 Hz, 3H), 3.30-3.32 (m, 4H), 3.66-3.68 (m, 4H), 4.47 (q, J = 7.12 Hz, 2H), 7.16- 7.21 (m, 2H), 7.65 (ddd, J = 2.88, 4.46, 9.09 Hz, 1H), 8.05 (br s, 1H), 8.20 (ddd, J = 2.80, 5.44 Hz, 1H), 8.39 (t, J = 2.12 Hz, 1H), 8.87 (br s, 1H), 9.19 (br s, 1H). | 5-Amino-2- fluorobenzo nitrile |
| Example 418^{b} | 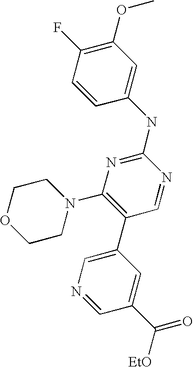 | The compound was taken to the next step on the basis of Mass spectrum with 78% purity. MS(ES): 454 (M + 1) for C₂₃H₂₄FN₅O₄. | 4-Fluoro-3- methoxy- aniline |
| Example 419^{c} | 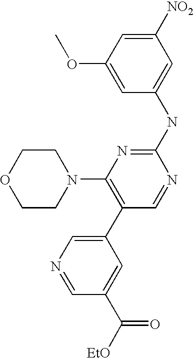 | MS(ES): 481 (M + 1) for C₂₃H₂₄N₆O₆. 400 MHz, DMSO-d₆: δ 1.35 (t, J = 7.08 Hz, 3H), 3.40 (br s, 4H), 3.58 (br s, 4H), 3.89 (s, 3H), 4.38 (q, J = 7.12 Hz, 2H), 7.44 (t, J = 2.00 Hz, 1H), 7.61 (d, J = 2.00 Hz, 1H), 8.24 (d, J = 6.40 Hz, 1H), 8.44-8.46 (m, 2H), 8.96 (d, J = 2.00 Hz, 1H), 9.09 (d, J = 2.00 Hz, 1H), 10.85 (br s, 1H). | 3-Methoxy- 5-nitro aniline |
| Example 420^{b} | 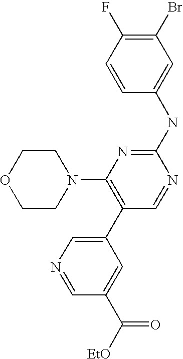 | MS(ES): 502 (M) and 504 (M + 2) for C₂₂H₂₁BrFN₅O₃. 400 MHz, DMSO-d₆: δ 1.35 (t, J = 7.04 Hz, 3H), 3.22 (br s, 4H), 3.55 (br s, 4H), 4.37 (q, J = 7.12 Hz, 2H), 7.30 (t, J = 8.80 Hz, 1H), 7.63-7.66 (m, 1H), 8.18 (s, 1H), 8.30 (dd, J = 2.20, 6.32 Hz, 1H), 8.38 (br s, 1H), 8.93 (br s, 1H), 9.00 (br s, 1H), 9.73 (br s, 1H). | 3-bromo-4- fluoro- aniline |
| Example 421^{b} | 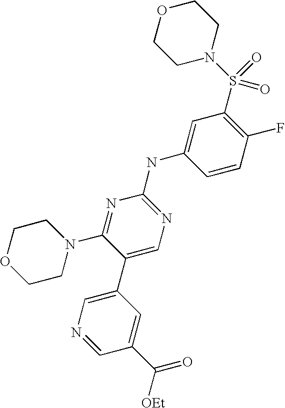 | MS(ES): 573 (M + 1) for C₂₆H₂₉FN₆O₆S. 400 MHz, DMSO-d₆: δ 1.35 (t, J = 7.08 Hz, 3H), 3.04 (br s, 4H), 3.31 (br s, 4H), 3.54-3.56 (m, 4H), 3.64-3.66 (m, 4H), 4.38 (q, J = 7.12 Hz, 2H), 7.50 (t, J = 9.32 Hz, 1H), 7.82-7.84 (m, 1H), 8.19 (s, 1H), 8.40 (t, J = 2.08 Hz, 1H), 8.50 (dd, J = 2.44, 5.68 Hz, 1H), 8.93 (d, J = 2.16 Hz, 1H), 9.05 (d, J = 1.96 Hz, 1H), 10.32 (br s, 1H). | 4-Fluoro-3- (morpholine- 4- sulfonyl)- phenylamine |
| Example 422^{b} | 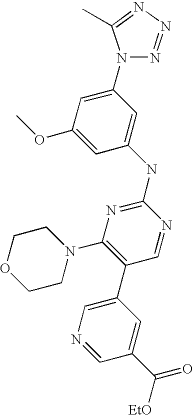 | MS(ES): 518 (M + 1) for C₂₅H₂₇N₉O₄. 400 MHz, DMSO-d₆: δ 1.36 (t, J = 6.80 Hz, 3H), 2.61 (s, 3H), 3.23 (br s, 4H), 3.52-3.54 (m, 4H), 3.83 (s, 3H), 4.38 (q, J = 7.20 Hz, 2H), 6.88 (s, 1H), 7.63 (s, 1H), 7.79 (d, J = 1.60 Hz, 1H), 8.22 (s, 1H), 8.40 (s, 1H), 8.95 (d, J = 2.00 Hz, 1H), 9.02 (d, J = 2.00 Hz, 1H), 9.92 (s, 1H). | 3-Methoxy- 5-(5- methyl- tetrazol-1- yl)- phenylamine |
| Example 423^{c} | 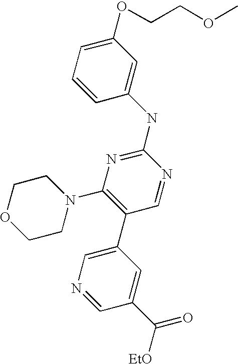 | MS(ES): 480 (M + 1) for C₂₅H₂₉N₅O₅. 400 MHz, DMSO-d₆: δ 1.36 (t, J = 6.80 Hz, 3H), 3.32 (s, 3H), 3.44 (br s, 4H), 3.57 (br s, 4H), 3.66-3.69 (m, 2H), 4.09- 4.11 (m, 3H), 4.40 (q, J = 7.20 Hz, 2H), 6.70-6.80 (m, 1H), 7.13 (d, J = 0.80 Hz, 1H), 7.26-7.32 (m, 2H), 8.19 (s, 1H), 8.40 (t, J = 2.00 Hz, 1H), 8.93 (d, J = 2.40 Hz, 1H), 9.10 (d, J = 2.00 Hz,1H), 10.90 (br s, 1H). | 3-(2- Methoxy- ethoxy)- phenylamine |

Solvents used in the reaction
^{a}n-butanol
^{b}acetonitrile
^{c}dioxane

### General procedure for the reaction of anilines with 3-[3-(2-Chloro-4-morpholin-4-yl-pyrimidin-5-yl)-phenyl]-acrylic acid ethyl ester (Intermediate 153)

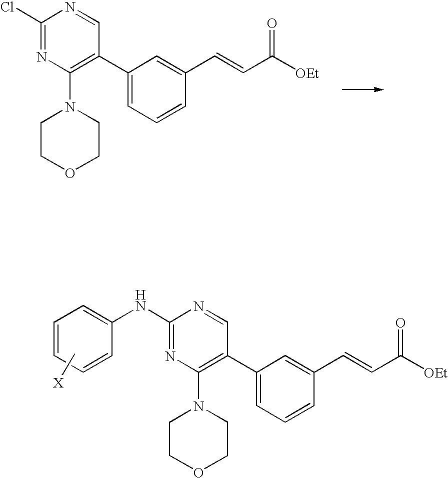

To a suspension of 3-[3-(2-Chloro-4-morpholin-4-yl-pyrimidin-5-yl)-phenyl]-acrylic acid ethyl ester Intermediate 153 (1 eq.) taken in n-BuOH/acetonitrile/dioxane was added the corresponding aniline. The reaction mixture was then treated with 4 N HCl in dioxane and refluxed at 100° C. for 1.5 h. The reaction mixture was cooled to room temperature, diluted with diethyl ether and the solid was filtered and dried to afford the corresponding cinnamic acid ethyl ester.

The compounds in the below table were prepared using this general procedure with the starting material and solvent specified.

| Compound | Structure | Mass spectrum and ¹H NMR | SM |
|---|---|---|---|
| Example 424^{b} | 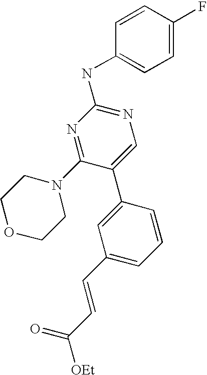 | MS(ES): 449.2 (M + 1) for C₂₅H₂₅FN₄O₃. 300 MHz, DMSO-d₆ : δ 1.27 (t, J = 7.14 Hz, 3H), 3.20-3.25 (m, 4H), 3.50-3.55 (m, 4H), 4.20 (q, J = 7.17 Hz, 2H), 6.71 (d, J = 16.08 Hz, 1H), 7.07-7.13 (m, 2H), 7.45-7.55 (m, 3H), 7.60-7.80 (m, 4H), 8.04 (s, 1H), 9.42 (s, 1H). | 4-fluoro aniline |
| Example 425^{b} | 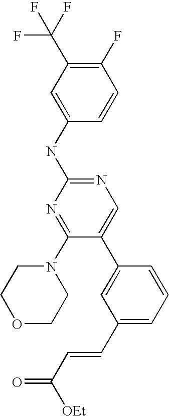 | MS(ES): 517 (M + 1) for C₂₆H₂₄F₄N₄O₃. 300 MHz, DMSO-d₆: δ 1.25 (t, J = 7.05 Hz, 1H), 3.23-3.32 (m, 4H), 3.54-3.55 (m, 4H), 4.19 (q, J = 7.02 Hz, 2H), 6.72 (d, J = 16.05 Hz, 1H), 7.39-7.55 (m, 3H), 7.69 (d, J = 16.23 Hz, 1H), 7.66 (br s, 1H), 7.82 (s, 1H), 7.87 (br s, 1H), 8.09 (s, 1H), 8.44 (d, J = 4.29 Hz, 1H), 9.79 (s, 1H). | 4-fluoro-3- trifluoro- methylaniline |
| Example 426^{b} | 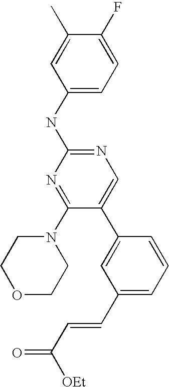 | MS(ES): 463 (M + 1) for C₂₆H₂₇FN₄O₃. 400 MHz, DMSO-d₆: δ 1.26 (t, J = 7.08 Hz, 3H), 2.07 (s, 3H), 3.32-3.48 (m, 4H), 3.50-3.70 (m, 4H), 4.20 (q, J = 7.12 Hz, 2H), 6.74 (d, J = 16.04 Hz, 1H), 7.17 (t, J = 9.04 Hz, 1H), 7.21-7.27 (m, 1H), 7.38-7.41 (m, 1H), 7.47-7.54 (m, 3H), 7.70 (d, J = 16.08 Hz, 1H), 7.74 (d, J = 7.36 Hz, 1H), 7.81 (br s, 1H), 7.99 (br s, 1H). | 4-fluoro-3- methylaniline |
| Example 427^{b} | 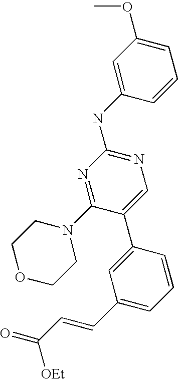 | MS(ES): 461 (M + 1) for C₂₆H₂₈N₄O₄. 300 MHz, DMSO-d₆: δ 1.25 (t, J = 7.05 Hz, 3H), 3.23 (br s, 4H), 3.54 (br s, 4H), 3.73 (s, 3H), 4.19 (q, J = 7.17 Hz, 2H), 6.49 (d, J = 8.16 Hz, 1H), 6.71 (d, J = 16.23 Hz, 1H), 7.14 (t, J = 7.86 Hz, 1H), 7.27 (d, J = 7.83 Hz, 1H), 7.49-7.66 (m, 3H), 7.72- 7.82 (m, 2H), 7.99 (s, 1H), 8.06 (s, 1H), 8.30 (s, 1H). | m-anisidine |
| Example 428^{b} | 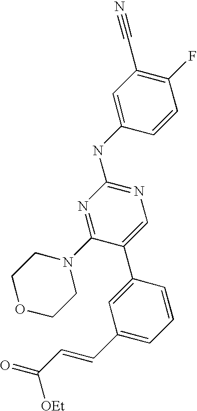 | MS(ES): 474 (M + 1) for C₂₆H₂₄FN₅O₃. 300 MHz, DMSO-d₆ : δ 1.25 (t, J =7.05 Hz, 3H), 3.32 (br s, 4H), 3.55 (br s, 4H), 4.19 (q, J = 6.75 Hz, 2H), 6.73 (d, J = 16.11 Hz, 1H), 7.08 (s, 1H), 7.25 (br s, 1H), 7.41 (br s, 1H), 7.50-7.56 (m, 2H), 7.66-7.72 (m, 1H),7.81 (s, 1H), 7.95 (br s, 1H), 8.07 (s, 1H), 8.22 (br s, 1H). | 5-Amino-2- fluoro- benzonitrile |
| Example 429^{b} | 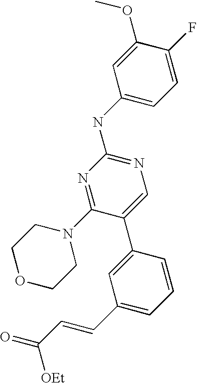 | MS(ES): 479 (M + 1) for C₂₆H₂₇FN₄O₄. 400 MHz, DMSO-d₆: δ 1.26 (t, J = 7.08 Hz, 3H), 3.41 (br s, 4H), 3.50 (br s, 4H), 3.84 (s, 3H), 4.20 (q, J = 7.12 Hz, 2H), 6.75 (d, J = 16.04 Hz, 1H), 7.04-7.07 (m, 1H), 7.24 (dd, J = 8.80, 11.26 Hz, 1H), 7.48-7.55 (m, 3H), 7.72-7.76 (m, 1H), 7.82 (s, 1H), 8.01 (s, 1H), 10.39 (br s, 1H). | 4-Fluoro-3- methoxy- aniline |
| Example 430^{c} | 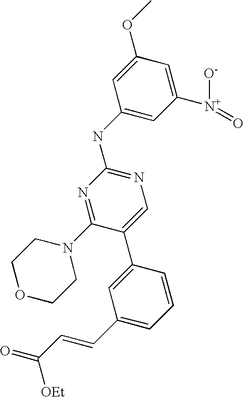 | MS(ES): 506 (M + 1) for C₂₆H₂₇N₅O₆. 400 MHz, DMSO-d₆ : δ 1.26 (t, J = 7.04 Hz, 3H), 3.28-3.29 (m, 4H), 3.55-3.57 (m, 4H), 3.85 (s, 3H), 4.19 (q, J = 7.12 Hz, 2H), 6.73 (d, J = 16.04 Hz, 1H), 7.29 (t, J = 2.20 Hz, 1H), 7.48 (t, J = 7.60 Hz, 1H), 7.55 (d, J = 7.80 Hz, 1H),7.67-7.71 (m, 3H), 7.84 (br s, 1H), 8.13 (s, 1H), 8.61 (t, J = 1.88 Hz, 1H), 9.93 (br s, 1H). | 3-Methoxy- 5-nitro- phenylamine |
| Example 431^{b} | 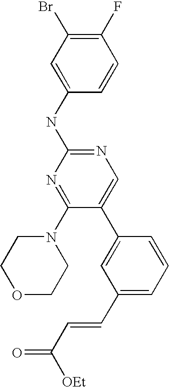 | MS(ES): 527 (M) and 529 (M + 2) for C₂₅H₂₄BrFN₄O₃. 300 MHz, DMSO-d₆ : δ 1.25 (t, J = 6.99 Hz, 3H), 3.23 (br s, 4H), 3.55 (br s, 4H), 4.19 (q, J = 6.87 Hz, 2H), 6.71 (d, J = 16.11 Hz, 1H), 7.29 (t, J = 8.94 Hz, 1H), 7.47-7.54 (m, 2H), 7.55-7.65 (m, 2H), 7.69 (d, J = 16.32 Hz, 1H), 7.81 (s, 1H), 8.08 (s, 1H), 8.30 (dd, J = 2.28, 6.24 Hz, 1H), 9.62 (br s, 1H). | 3-Bromo-4- fluoroaniline |
| Example 432^{b} | 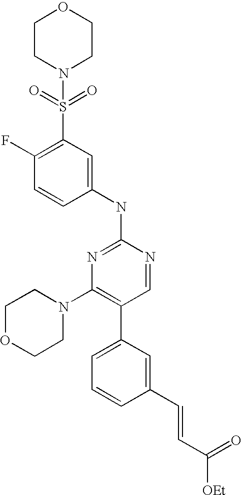 | MS(ES): 598 (M + 1) for C₂₉H₃₂FN₅O₆S. 400 MHz, DMSO-d₆: δ 1.25 (t, J = 7.04 Hz, 3H), 3.04 (br s, 4H), 3.37 (br s, 4H), 3.55-3.65 (m, 4H), 3.63- 3.66 (m, 4H), 4.19 (q, J = 7.04 Hz, 2H), 6.74 (d, J = 16.00 Hz, 1H), 7.49-7.54 (m, 3H), 7.69 (d, J = 16.04 Hz, 1H), 7.72 (br s, 1H), 7.79-7.80 (m, 1H), 7.82 (br s, 1H), 8.07 (s, 1H), 8.43-8.44 (m, 1H), 10.46 (br s, 1H). | 4-Fluoro-3- (morpholine- 4-sulfonyl)- phenylamine |
| Example 433^{b} | 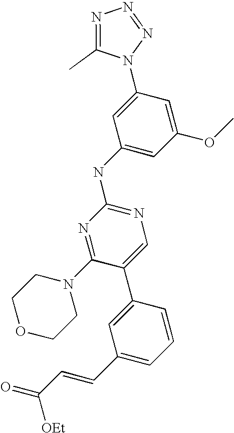 | MS(ES): 543 (M + 1) for C₂₈H₃₀N₈O₄. 400 MHz, DMSO-d₆: δ 1.26 (t, J = 7.12 Hz, 3H), 2.60 (s, 3H), 3.22-3.24 (m, 4H), 3.51-3.53 (m, 4H), 3.82 (s, 3H), 4.20 (q, J = 7.12 Hz, 2H), 6.72 (d, J = 16.04 Hz, 1H), 6.85 (t, J = 2.12 Hz, 1H), 7.48 (t, J = 7.64 Hz, 1H), 7.54 (d, J = 7.08 Hz, 1H), 7.62 (t, J = 2.04 Hz, 1H), 7.67 (d, J = 7.48 Hz, 1H), 7.69 (d, J = 16.00 Hz, 1H), 7.78 (t, J = 1.84 Hz, 1H), 7.83 (s, 1H), 8.11 (s, 1H), 9.79 (s, 1H). | 3-methoxy- 5-(5-methyl- 1H-tetrazol- 1-yl)aniline |
| Example 434^{c} | 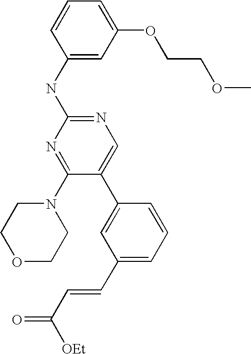 | MS(ES): 505 (M + 1) for C₂₈H₃₂N₄O₅. 400 MHz, DMSO-d₆: δ 1.27 (t, J = 7.20 Hz, 3H), 3.24 (br s, 4H), 3.32 (s, 3H), 3.55-3.56 (m, 4H), 3.67 (t, J = 3.60 Hz, 2H), 4.02-4.07 (m, 2H), 4.21 (q, J = 7.20 Hz, 2H), 6.51 (d, J = 8.40 Hz, 1H), 6.73 (d, J = 16.00 Hz, 1H), 7.16 (t, J = 8.00 Hz, 1H), 7.29 (d, J = 8.00 Hz, 1H), 71.51 (t, J = 7.60 Hz, 1H), 7.54-7.56 (m, 1H), 7.66 (s, 1H), 7.70 (d, J = 16.80 Hz, 1H), 7.80 (br s, 1H), 8.08 (s, 1H), 8.32 (d, J = 1.20 Hz, 1H), 9.41 (br s, 1H). | 3-(2- methoxy- ethoxy)- aniline |

Solvents used in the reaction
^{a}n-butanol
^{b}acetonitrile
^{c}dioxane

### General Procedure for the Hydrolysis of Pyridyl Ester Derivatives

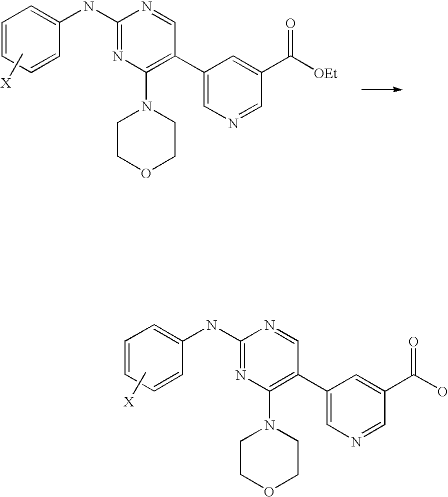

Ester compound (1 eq, 0.22 mmol) was dissolved in a mixture of tetrahydrofuran (1 mL) and water (1 mL) and treated with sodium hydroxide (35 mg, 1N, 0.88 mmol). The reaction was allowed to stir at room temperature for 1 hr. After completion of reaction, the reaction mixture was carefully acidified with 1 N HCl. The solid that precipitated was filtered off, washed with water and dried under vacuum.

The compounds in the below table were prepared using this general procedure and the specified starting material.

| Compound | Structure | Mass spectrum and ¹H NMR | SM |
|---|---|---|---|
| Example 435 | 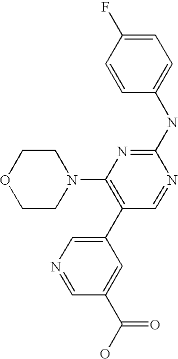 | MS(ES): 396 (M + 1) for C₂₀H₁₈FN₅O₃. 400 MHz, DMSO-d₆: δ 3.20-3.21 (m, 4H), 3.54-3.56 (m, 4H), 7.12 (t, J = 8.92 Hz, 2H), 7.73-7.77 (m, 2H), 8.13 (s, 1H), 8.34 (t, J = 1.96 Hz, 1H),8.89 (d, J = 2.00 Hz, 1H), 8.98 (d, J = 1.68 Hz, 1H), 9.53 (s, 1H), 13.60 (br s, 1H). | 5-[2-(4-Fluoro- phenylamino)- 4-morpholin-4- yl-pyrimidin-5- yl]-nicotinic acid ethyl ester Example 413 |
| Example 436 | 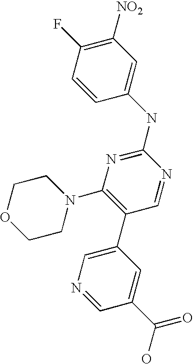 | MS(ES): 441 (M + 1) for C₂₀H₁₇FN₆O₅. 400 MHz, DMSO-d₆: δ 3.25-3.27 (m, 4H), 3.56-3.57 (m, 4H), 7.53 (dd, J = 9.24, 11.08 Hz, 1H), 7.90-7.92 (m, 1H), 8.20 (s, 1H), 8.35 (d, J = 1.92 Hz, 1H), 8.91-9.00 (m, 3H), 10.03 (s, 1H), 13.60 (br s, 1H). | 5-[2-(4-Fluoro- 3-nitro- phenylamino)- 4-morpholin-4- yl-pyrimidin-5- yl]-nicotinic acid ethyl ester Example 411 |
| Example 437 | 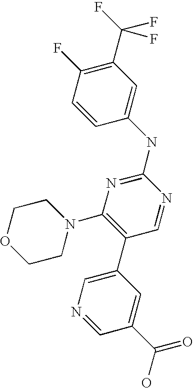 | MS(ES): 464 (M + 1) for C₂₁H₁₇F₄N₅O₃. 400 MHz, DMSO-d₆: δ 3.22-3.23 (m, 4H), 3.54-3.56 (m, 4H), 7.44 (t, J = 9.76 Hz, 1H), 7.89-7.92 (m, 1H), 8.18 (s, 1H), 8.35 (m, 1H), 8.43-8.44 (m, 1H), 8.91 (d, J = 2.04 Hz, 1H), 8.99 (d, J = 1.80 Hz, 1H), 9.89 (br s, 1H), 13.53 (br s, 1H). | 5-[2-(4-Fluoro- 3- trifluoromethyl- phenylamino)- 4-morpholin-4- yl-pyrimidin-5- yl] -nicotinic acid ethyl ester Example 414 |
| Example 438 | 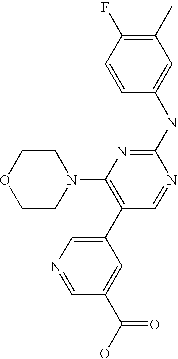 | MS(ES): 410 (M + 1) for C₂₁H₂₀FN₅O₃. 400 MHz, DMSO-d₆: δ 2.22 (s, 3H), 3.21-3.22 (m, 4H), 3.55-3.56 (m, 4H), 7.05 (t, J = 9.20 Hz, 1H), 7.54 (dd, J = 4.40, 8.20 Hz, 1H), 7.70 (dd, J = 2.40, 7.20 Hz, 1H), 8.14 (s, 1H), 8.35 (s, 1H), 8.91 (br s, 1H), 8.99 (br s, 1H), 9.49 (br s, 1H), 13.60 (br s, 1H). | 5-[2-(4-Fluoro- 3-methyl- phenylamino)- 4-morpholin-4- yl-pyrimidin-5- yl] -nicotinic acid ethyl ester Example 415 |
| Example 439 | 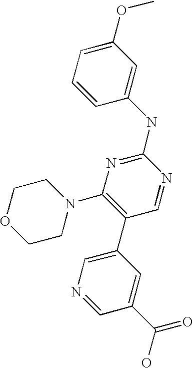 | MS(ES): 408 (M + 1) for C₂₁H₂₁N₅O₄. 400 MHz, DMSO-d₆: δ 3.22 (br s, 4H), 3.55 (br s, 4H), 3.73 (s, 3H), 6.51 (d, J = 7.96 Hz, 1H),7.16 (t, J = 8.12 Hz, 1H), 7.28 (d, J = 7.88 Hz, 1H), 7.54 (br s, 1H), 8.15 (br s, 1H), 8.35 (br s, 1H), 8.91 (br s, 1H), 8.98 (br s, 1H), 9.51 (br s, 1H), 13.6 (br s, 1H). | 5-[2-(3- Methoxy- phenylamino)- 4-morpholin-4- yl-pyrimidin-5- yl] -nicotinic acid ethyl ester Example 416 |
| Example 440 | 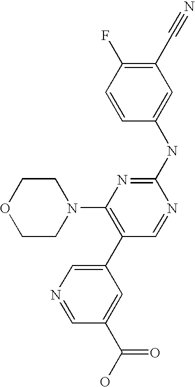 | MS(ES): 421 (M + 1) for C₂₁H₁₇FN₆O₃. 400 MHz, DMSO-d₆: δ 3.22-3.23 (m, 4H), 3.53-3.56 (m, 4H), 7.47 (t, 9.24 Hz, 1H), 7.96-8.02 (m, 1H), 8.19 (s, 1H), 8.31-8.35 (m, 2H), 8.89 (d, J = 2.04 Hz, 1H), 8.99 (d, J = 1.76 Hz, 1H), 9.91 (s, 1H), 13.50 (br s, 1H). | 5-[2-(3-Cyano- 4-fluoro- phenylamino)- 4-morpholin-4- yl-pyrimidin-5- yl]-nicotinic acid ethyl ester Example 417 |
| Example 441 | 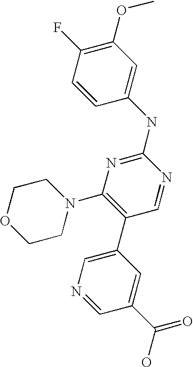 | MS(ES): 426 (M + 1) for C₂₁H₂₀FN₅O₄. 400 MHz, DMSO-d₆ : δ 3.22 (t, J = 4.08 Hz, 4H), 3.55 (t, J = 4.24 Hz, 4H), 3.83 (s, 3H), 7.10 (dd, J = 8.80, 11.24 Hz, 1H), 7.20 (t, J = 2.44 Hz, 1H), 7.76 (dd, J = 2.08, 7.96 Hz, 1H), 8.15 (s, 1H), 8.55 (d, J = 1.84 Hz, 1H), 8.90 (s, 1H), 8.98 (s, 1H), 9.52 (s, 1H), 13.50 (br s, 1H). | 5-[2-(4-Fluoro- 3-methoxy- phenylamino)- 4-morpholin-4- yl-pyrimidin-5- yl]-nicotinic acid ethyl ester Example 418 |
| Example 442 | 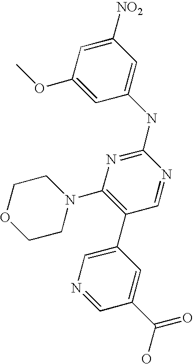 | MS(ES): 453 (M + 1) for C₂₁H₂₀N₆O₆. 400 MHz, DMSO-d₆: δ 3.28-3.29 (m, 4H), 3.57-3.58 (m, 4H), 3.86 (s, 3H), 7.31 (s, 1H), 7.72 (s, 1H), 8.22 (s, 1H), 8.37 (s, 1H), 8.61 (s, 1H), 8.93 (d, J = 1.20 Hz, 1H), 9.01 (s, 1H), 10.04 (s, 1H), 13.58 (br s, 1H). | 5-[2-(3- Methoxy-5- nitro- phenylamino)- 4-morpholin-4- yl-pyrimidin-5- yl]-nicotinic acid ethyl ester Example 419 |
| Example 443 | 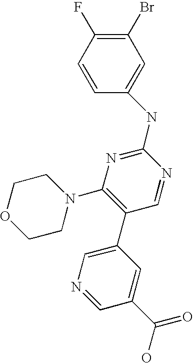 | MS(ES): 474 (M) and 476 (M + 2) for C₂₀H₁₇BrFN₅O₃. 400 MHz, DMSO-d₆: δ 3.23-3.38 (m, 4H), 3.51-3.52 (m, 4H), 7.30 (t, J = 8.84 Hz, 1H), 7.63-7.67 (m, 1H), 8.17 (s, 1H), 8.30 (dd, J = 2.52, 6.40 Hz, 1H), 8.34 (d, J = 1.84 Hz, 1H), 8.90 (s, 1H), 8.99 (s, 1H), 9.72 (s, 1H), 13.50 (s, 1H). | 5-[2-(3-Bromo- 4-fluoro- phenylamino)- 4-morpholin-4- yl-pyrimidin-5- yl]-nicotinic acid ethyl ester Example 420 |
| Example 444 | 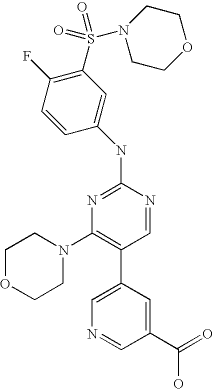 | MS(ES): 545 (M + 1) for C₂₄H₂₅FN₆O₆S. 400 MHz, DMSO-d₆: δ 3.03 (br s, 4H), 3.24 (br s, 4H), 3.55 (br s, 4H), 3.65 (br s, 4H), 7.44 (t, J = 9.52 Hz, 1H), 7.83-7.85 (m, 1H), 8.17 (s, 1H), 8.34 (s, 1H), 8.59 (br s, 1H), 8.90 (s, 1H), 8.99 (s, 1H), 9.94 (s, 1H), 13.58 (br s, 1H). | 5-{2-[4-Fluoro- 3-(morpholine- 4-sulfonyl)- phenylamino]- 4-morpholin-4- yl-pyrimidin-5- yl} -nicotinic acid ethyl ester Example 421 |
| Example 445 | 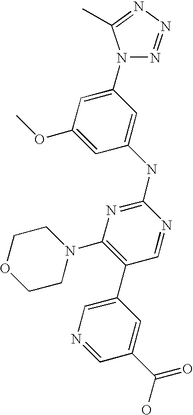 | MS(ES): 490 (M + 1) for C₂₃H₂₃N₉O₄. 400 MHz, DMSO-d₆: δ 2.60 (s, 3H), 3.22-3.23 (m, 4H), 3.52-3.54 (m, 4H), 3.82 (s, 3H), 6.86 (t, J = 1.88 Hz, 1H), 7.62 (s, 1H), 7.78 (s, 1H), 8.19 (s, 1H), 8.34 (t, J = 1.96 Hz, 1H), 8.90 (d, J = 2.00 Hz, 1H), 8.99 (d, J = 1.72 Hz, 1H), 9.88 (s, 1H), 13.60 (br s, 1H). | 5-{2-[3- Methoxy-5-(5- methyl-tetrazol- 1-yl)- phenylamino]- 4-morpholin-4- yl-pyrimidin-5- yl}-nicotinic acid ethyl ester Example 422 |
| Example 446 | 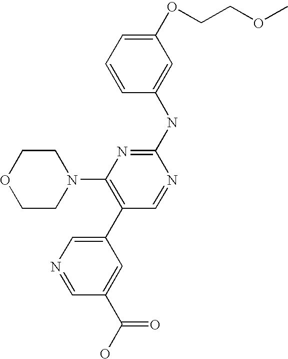 | MS(ES): 452 (M + 1) for C₂₃H₂₅N₅O₅. 400 MHz, DMSO-d₆: δ 3.22-3.23 (m, 4H), 3.31 (s merged with solvent peak, 3H), 3.54-3.56 (m, 4H), 3.65 (t, J = 4.52 Hz, 2H), 4.05 (t, J = 4.84 Hz, 2H), 6.52 (dd, J = 2.24, 8.08 Hz, 1H), 7.13-7.17 (m, 1H), 7.28 (d, J = 8.04 Hz, 1H), 7.53 (s, 1H), 8.15 (s, 1H), 8.35 (t, J = 2.08 Hz, 1H), 8.90 (d, J = 2.16 Hz, 1H), 8.98 (d, J = 1.92 Hz, 1H), 9.51 (s, 1H), 13.52 (br s, 1H). | 5-{2-[3-(2- Methoxy- ethoxy)- phenylamino]- 4-morpholin-4- yl-pyrimidin-5- yl}-nicotinic acid ethyl ester Example 423 |

### General Procedure for the Hydrolysis of Cinnamyl Ester Derivatives

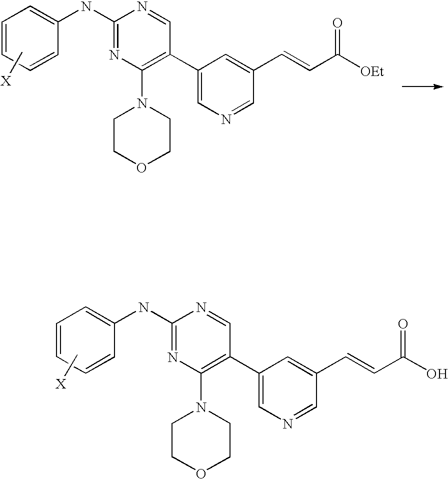

Ester compound (1 eq, 0.22 mmol) was dissolved in a mixture of tetrahydrofuran (1 mL) and water (1 mL) and treated with sodium hydroxide (35 mg, 1N, 0.88 mmol). The reaction was allowed to stir at room temperature for 1 hr. After completion of reaction, the reaction mixture was carefully acidified with 1 N HCl. The solid that precipitated was filtered off, washed with water and dried under vacuum.

The compounds in the below table were prepared using this general procedure and the specified starting material.

| Compound | Structure | Mass spectrum and ¹H NMR | SM |
|---|---|---|---|
| Example 447 | 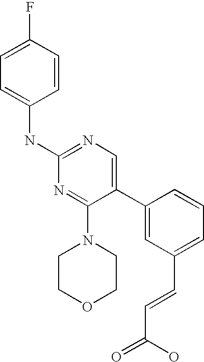 | MS(ES): 421 (M + 1) for C₂₃H₂₁FN₄O₃. 400 MHz, DMSO-d₆: δ 3.21-3.22 (m, 4H), 3.53-3.54 (m, 4H), 6.61 (d, J = 15.92 Hz, 1H), 7.11 (t, J = 8.88 Hz, 2H),7.44-7.48 (m, 2H), 7.54-7.60 (m, 2H), 7.74-7.77 (m, 3H), 8.04 (s, 1H), 9.42 (s, 1H). | 3-{3-[2-(4- Fluoro- phenylamino)-4- morpholin-4-yl- pyrimidin-5-yl]- phenyl}-acrylic acid ethyl ester Example 424 |
| Example 448 | 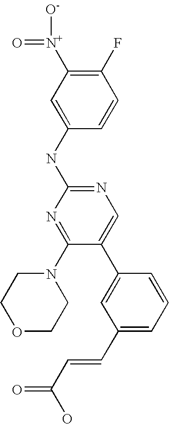 | MS(ES): 466 (M + 1) for C₂₃H₂₀FN₅O₅. 400 MHz, DMSO-d₆: δ 3.27-3.28 (m, 4H), 3.55-3.57 (m, 4H), 6.62 (d, J = 16.08 Hz, 1H), 7.47-7.54 (m, 3H), 7.64-7.66 (m, 1H), 7.64 (d, J = 15.92 Hz, 1H), 7.80 (br s, 1H), 7.88-7.92 (m, 1H), 8.12 (s, 1H), 8.96 (dd, J = 2.76, 6.88 Hz, 1H), 9.94 (br s, 1H), 12.44 (br s, 1H). | 3-{3-[2-(4- Fluoro-3-nitro- phenylamino)-4- morpholin-4-yl- pyrimidin-5-yl]- phenyl}-acrylic acid ethyl ester Example 412 |
| Example 449 | 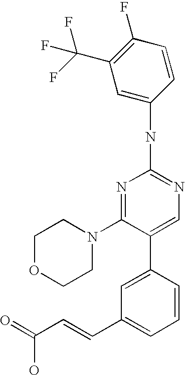 | MS(ES): 489 (M + 1) for C₂₄H₂₀F₄N₄O₃. 400 MHz, (CD₃)CO₂D: δ 3.63 (br s, 4H), 3.74-3.76 (m, 4H), 6.68 (d, J = 16.20 Hz, 2H), 7.36 (t, J = 9.44 Hz, 1H), 7.54 (d, J = 7.24 Hz, 1H), 7.60 (t, J = 7.44 Hz, 1H), 7.73 (s, 1H), 7.73 (br s, 1H), 7.79-7.81 (m, 1H), 7.88 (d, J = 16.00 Hz, 1H), 8.08 (s, 1H), 8.33 (d, J = 5.08 Hz, 1H). | 3-{3-[2-(4- Fluoro-3- trifluoromethyl- phenylamino)-4- morpholin-4-yl- pyrimidin-5-yl]- phenyl}-acrylic acid ethyl ester Example 425 |
| Example 450 | 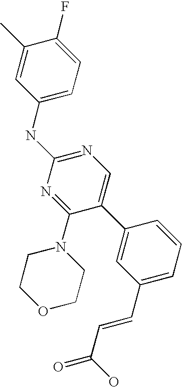 | MS(ES): 435 (M + 1) for C₂₄H₂₃FN₄O₃. 400 MHz, DMSO-d₆: δ 2.21 (s, 3H), 3.22-3.23 (m, 4H), 3.55-3.58 (m, 4H), 6.61 (d, J = 16.00 Hz, 1H), 7.04 (t, J = 9.20 Hz, 1H), 7.46-7.55 (m, 3H), 7.64 (d, J = 16.00 Hz, 1H), 7.64 (s, 1H), 7.70 (dd, J = 2.40, 6.80 Hz, 1H), 7.78 (br s, 1H), 8.05 (br s, 1H), 9.37 (br s, 1H), 12.40 (br s, 1H). | 3-{3-[2-(4- Fluoro-3-methyl- phenylamino)-4- morpholin-4-yl- pyrimidin-5-yl]- phenyl}-acrylic acid ethyl ester Example 426 |
| Example 451 | 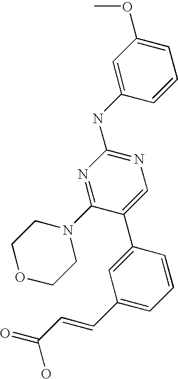 | MS(ES): 433 (M + 1) for C₂₄H₂₄N₄O₄. 400 MHz, DMSO-d₆: δ 3.24 (br s, 4H), 3.55 (br s, 4H), 3.73 (s, 3H), 6.50 (d, J = 6.80 Hz, 1H), 6.61 (d, J = 16.04 Hz, 1H), 7.15 (t, J = 6.92 Hz, 1H), 7.27 (d, J = 5.20 Hz, 1H), 7.47- 7.65 (m, 5H), 7.79 (br s, 1H), 8.06 (br s, 1H), 9.40 (br s, 1H), 12.42 (br s, 1H). | 3-{3-[2-(3- Methoxy- phenylamino)-4- morpholin-4-yl- pyrimidin-5-yl]- phenyl}-acrylic acid ethyl ester Example 427 |
| Example 452 | 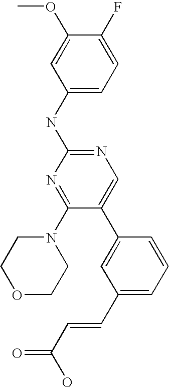 | MS(ES): 451 (M + 1) for C₂₄H₂₃FN₄O₄. 400 MHz, DMSO-d6 : δ 3.21-3.27 (m, 4H), 3.52-3.58 (m, 4H), 3.83 (s, 3H), 6.61 (d, J = 16.00 Hz, 1H), 7.09 (dd, J = 8.88, 11.24 Hz, 1H), 7.18-7.20 (m, 1H), 7.45-7.53 (m, 2H), 7.62 (d, J = 15.92 Hz, 1H), 7.62 (d, J = 7.60 Hz, 1H), 7.75-7.78 (m, 2H), 8.06 (s, 1H), 9.41 (s, 1H), 12.50 (br s, 1H). | 3-{3-[2-(4- Fluoro-3- methoxy- phenylamino)-4- morpholin-4- pyrimidin-5-yl]- phenyl}-acrylic acid ethyl ester Example 429 |
| Example 453 | 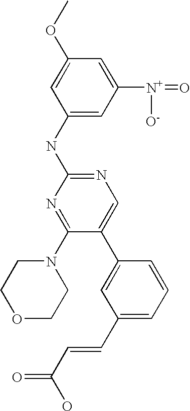 | MS(ES): 478 (M + 1) for C₂₄H₂₃N₅O₆. 400 MHz, DMSO-d₆: δ 3.32 (br s, 4H), 3.56 (br s, 4H), 3.85 (s, 3H), 6.55 (d, J = 15.92 Hz, 1H), 7.28 (t, J = 2.20 Hz, 1H), 7.32 (d, J = 15.96 Hz, 1H), 7.43 (m, 2H), 7.50-7.51 (m, 1H), 7.66 (s, 1H), 7.72 (d, J = 1.56 Hz, 1H), 8.10 (d, J = 2.60 Hz, 1H), 8.60 (br s, 1H), 9.93 (br s, 1H). | 3-{3-[2-(3- Methoxy-5-nitro- phenylamino)-4- morpholin-4-yl- pyrimidin-5-yl]- phenyl}-acrylic acid ethyl ester Example 430 |
| Example 454 | 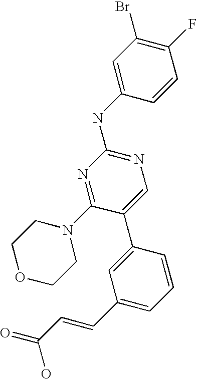 | MS(ES): 499 (M) and 501 (M + 2) for C₂₃H₂₀BrFN₄O₃. 400 MHz, DMSO-d₆: δ 3.25 (br s, 4H), 3.55 (br s, 4H), 6.61 (d, J = 16.08 Hz, 1H), 7.31 (t, J = 8.80 Hz, 1H), 7.48 (t, J = 7.44 Hz, 1H), 7.50 (br s, 1H), 7.63 (d, J = 15.96 Hz, 1H), 7.63- 7.65 (m, 2H), 7.78 (br s, 1H), 8.07 (s, 1H), 8.27 (dd, J = 1.96, 6.18 Hz, 1H), 9.71 (br s, 1H). | 3-{3-[2-(3- Bromo-4-fluoro- phenylamino)-4- morpholin-4-yl- pyrimidin-5-yl]- phenyl}-acrylic acid ethyl ester Example 431 |
| Example 455 | 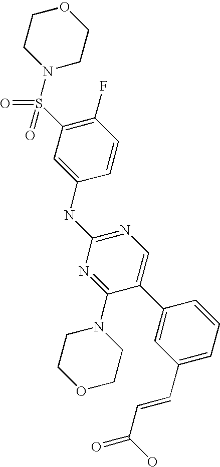 | MS(ES): 570 (M + 1) for C₂₇H₂₈FN₅O₆S. 400 MHz, DMSO-d₆: δ 3.03 (br s, 4H), 3.26 (br s, 4H), 3.54-3.55 (m, 4H), 3.64-3.65 (m, 4H), 6.61 (d, J = 16.04 Hz, 1H), 7.43 (t, J = 9.20 Hz, 1H), 7.48-7.53 (m, 2H), 7.63 (d, J = 15.76 Hz, 1H), 7.63 (s, 1H), 7.79 (s, 1H), 7.82-7.85 (m, 1H), 8.09 (s, 1H), 8.60-8.61 (m, 1H), 9.85 (s, 1H), 12.44 (br s, 1H). | 3-(3-{2-[4- Fluoro-3- (morpholine-4- sulfonyl)- phenylamino]-4- morpholin-4-yl- pyrimidin-5-yl}- phenyl)-acrylic acid ethyl ester Example 432 |
| Example 456 | 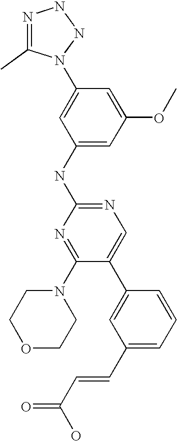 | MS(ES): 515 (M + 1) for C₂₆H₂₆N₈O_{4.} 400 MHz, DMSO-d₆: δ 2.61 (s, 3H), 3.27 (br s, 4H), 3.52-3.53 (m, 4H), 3.83 (s, 3H), 6.62 (d, J = 16.00 Hz, 1H), 6.89 (s, 1H), 7.49-7.54 (m, 2H), 7.60-7.67 (m, 3H), 7.76-7.80 (m, 2H), 8.10 (s, 1H), 9.89 (s, 1H), 12.40 (br s, 1H). | 3-(3-{2-[3- Methoxy-5-(5- methyl-tetrazol-1- yl)-phenylamino]- 4-morpholin-4- yl-pyrimidin-5- yl}-phenyl)- acrylic acid ethyl ester Example 433 |
| Example 457 | 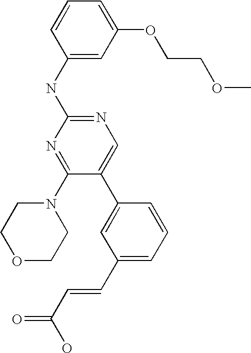 | MS(ES): 477 (M + 1) for C₂₆H₂₈N₄O₅. 400 MHz, DMSO-d₆: δ 3.24-3.25 (m, 4H), 3.32 (s, 3H), 3.55-3.55 (m, 4H), 3.66 (t, J = 4.40 Hz, 2H), 4.06 (t, J = 4.80 Hz, 2H), 6.50 (dd, J = 2.00, 8.00 Hz, 1H), 6.61 (d, J = 16.00 Hz, 1H), 7.15 (t, J = 8.40 Hz, 1H), 7.29 (d, J = 8.00 Hz, 1H), 7.45-7.53 (m, 2H), 7.55- 7.63 (m, 3H), 7.78 (s, 1H), 8.08 (s, 1H), 9.41 (br s, 1H). | 3-(3-{2-[ 3-(2- Methoxy- ethoxy)- phenylamino]-4- morpholin-4-yl- pyrimidin-5-yl]- phenyl)-acrylic acid ethyl ester Example 434 |

### Example 458

### (E)-3-(3-(2-(3-cyano-4-fluorophenylamino)-4-morpholinopyrimidin-5-yl)phenyl)acrylic acid

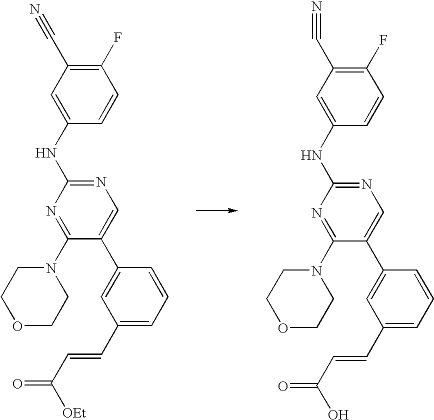

To 3-{3-[2-(3-Cyano-4-fluoro-phenylamino)-4-morpholin-4-yl-pyrimidin-5-yl]-phenyl}-acrylic acid ethyl ester (Example 428) (0.12 g, 0.25 mmol) in THF (1 mL) and water (0.2 mL), sodium hydroxide (0.5 mmol, 20 mg) was added and the mixture was heated at 60° C. overnight. The reaction mixture was then acidified using 1.5 N HCl and the solid obtained was filtered, washed with water and dried. LCMS analysis indicated that the solid was a 17:3 mixture of the title compound and the corresponding carboxamide resulting from nitrile hydrolysis. A pure sample of the title compound was produced by converting the carboxamide to the nitrile using the procedure below.

The mixture obtained as above (0.07 g) was taken in POCl₃ (1 mL) and heated at 100° C. for 3 h. The reaction mixture was cooled and concentrated under vacuo. Crushed ice was then added to the slurry to obtain an off-white solid, which was filtered, further washed with water and dried to give the pure title compound (0.06 g).

MS(ES): 446 (M+1) for C₂₄H₂₀FN₅O₃.

400 MHz, DMSO-d₆: δ 3.34 (br s, 4H), 3.55 (br s, 4H), 6.63 (d, J=16.04 Hz, 1H), 7.49-7.56 (m, 3H), 7.64 (d, J=16.04 Hz, 1H), 7.69 (d, J=6.72 Hz, 1H), 7.78 (s, 1H), 7.95 (br s, 1H), 8.06 (s, 1H), 8.21 (m, 1H), 10.36 (br s, 1H).

### Example 459

### 6-(2-(3-chloro-4-fluorophenylamino)-4-morpholinopyrimidin-5-yl)-4-oxo-4H-chromene-3-carboxylic acid

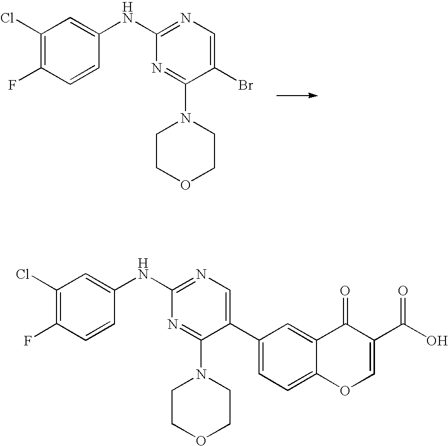

A suspension of 5-bromo-N-(3-chloro-4-fluorophenyl)-4-morpholin-4-ylpyrimidin-2-amine [Intermediate 146] (0.56 mmol, 220 mg), 2:1 mixture of boronic acid and pinacol boronate, Intermediate 155 (52 mg), tris(dibenzylideneacetone)dipalladium(0) (10 mol %, 0.054 mmol, 52 mg), 2-Dicyclohexylphosphino-2′,4′,6′-triisopropylbiphenyl (30 mol %, 0.17 mmol, 81 mg) and sodium carbonate (0.56 mmol, 60 mg) in 4:1 acetonitrile-water (10 mL) was degassed and then heated to 90° C. for 30 minutes. Solvent was removed in vacuo, resulting residue was redissolved in ethyl acetate (20 mL), filtered through a bed of celite and washed with water (2×10 mL). The filtrate was then acidified with 1.5N HCl, and the precipitate formed was filtered, washed with water and dried to yield the title compound as a brown solid (0.16 mmol, 80 mg, 29%).

MS(ES): 497 (M+1) for C₂₄H₁₈ClFN₄O₅.

400 MHz, DMSO-d₆: δ 3.23 (br s, 4H), 3.55 (br s, 4H), 7.25-7.30 (m, 1H), 7.40 (t, J=8.96 Hz, 1H), 7.54-7.58 (m, 1H), 7.62 (d, J=8.24 Hz, 1H), 7.91-7.93 (m, 1H), 7.97-8.00 (m, 2H), 9.92 (s, 1H), 9.98 (br s, 1H).

### Example 460

### N²-(3-chloro-4-fluorophenyl)-2′-methoxy-N⁴-[2-(pyridin-3-yl)ethyl]-5,5′-bipyrimidine-2,4-diamine

### Example 461

### N²-(3-chloro-4-fluorophenyl)-2′-methoxy-N⁴-[2-(pyridin-4-yl)ethyl]-5,5′-bipyrimidine-2,4-diamine

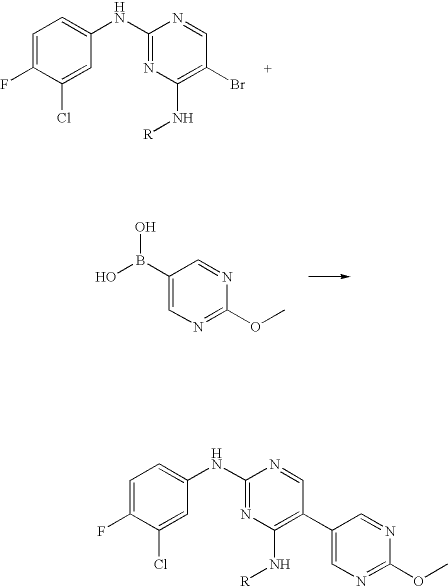

A suspension of 5-bromo-N-(3-chloro-4-fluorophenyl)-4-[alkylamino]pyrimidin-2-amine (1 eq), (2-methoxypyrimidin-5-yl)boronic acid (1.05 eq), tris(dibenzylideneacetone)dipalladium(0) (10 mol %), 2-dicyclohexylphosphino-2′,4′,6′-triisopropyl-1,1′-biphenyl (30 mol %) and sodium carbonate (1.1 eq) in acetonitrile/water (4:1) was heated to 90° C. for 30 minutes. The reaction mixture was diluted with ethyl acetate (25 mL); organic layer was separated, dried over sodium sulfate, filtered and concentrated. The resulting residue was purified by silica gel column chromatography using chloroform:methanol (9:1) as an eluent to afford the product.

The compounds in the below table were prepared using this method and the specified starting material.

| Compound | Structure | Mass and ¹H NMR data | SM |
|---|---|---|---|
| Example 460 | 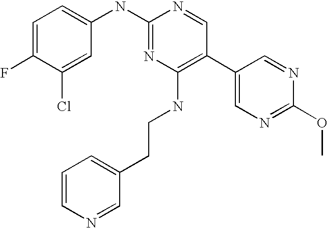 N²-(3-chloro-4-fluorophenyl)-2′-methoxy-N⁴-[2- (pyridin-3-yl)ethyl]-5,5′-bipyrimidin-2,4-diamine | MS(ES): 452 (M + 1) for C₂₂H₁₉ClFN₇O 400 MHz, DMSO-d₆: δ 2.89- 2.91 (t, J = 7.04 Hz, 2H), 3.58 (q, J = 6.72 Hz, 2H), 3.94 (s, 3H), 7.01 (t, J = 5.28 Hz, 1H), 7.25-7.32 (m, 2H), 7.61 (d, J = 6.92 Hz, 2H), 7.79 (s, 1H), 8.20 (dd, J = 2.52, 6.90 Hz, 1H), 8.40-8.43 (m, 2H), 8.48 (s, 2H), 9.43 (s, 1H). | Intermediate 45 5-bromo-N²-(3- chloro-4- fluorophenyl)-N⁴- [2-(pyridin-3- yl)ethyl]pyrimidine- 2,4-diamine |
| Example 461 | 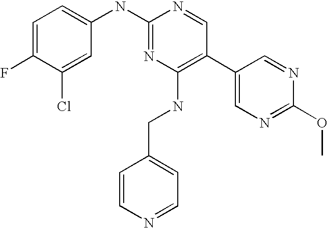 N²-(3-chloro-4-fluorophenyl)-2′-methoxy-N⁴-[2- (pyridin-4-yl)ethyl]-5,5′-bipyrimidine-2,4-diamine | MS(ES): 452 (M + 1) for C₂₂H₁₉ClFN₇O. 400 MHz, DMSO-d₆: δ 2.90 (t, J = 7.2 Hz, 2H), 3.60 (q, J = 6.40 Hz, 2H), 3.96 (s, 3H), 7.02 (t, J = 6.00 Hz, 1H), 7.23- 7.30 (m, 3H), 7.59-7.63 (m, 1H), 7.80 (s, 1H), 8.21 (dd, J = 2.40, 7.00 Hz, 1H), 8.46 (d, J = 6.00 Hz, 2H), 8.50 (s, 2H), 9.45 (s, 1H). | Intermediate 46 5-bromo-N²-(3- chloro-4- fluorophenyl)-N⁴- [2-(pyridin-4- yl)ethyl]pyrimidine- 2,4-diamine |

### Example 462

### N²-(3-chloro-4-fluorophenyl)-N⁴-[2-(1H-imidazol-4-yl)ethyl]-2′-methoxy-5,5′-bipyrimidine-2,4-diamine

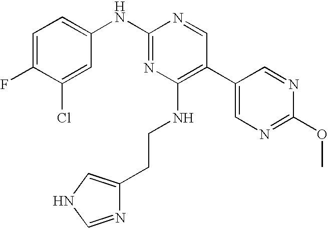

To a suspension of NaH (2.2 mmol, 60% dispersion in oil) in NMP, 2-(1H-Imidazol-4-yl)-ethylamine (1 eq) was added and stirred for 30 min. N-(3-chloro-4-fluorophenyl)-2′-methoxy-4-(methylsulfonyl)-5,5′-bipyrimidin-2-amine Intermediate 156 (1 eq) in NMP was added to the reaction mixture drop wise and stirred at RT for overnight. Water was added to the reaction mixture; the solid thus obtained was filtered and purified by column chromatography using chloroform-methanol to yield the title compound.

The compounds in the below table were prepared using this method and the specified starting material.

| Compound | Structure | Mass and ¹H NMR data | SM |
|---|---|---|---|
| Example 462 | 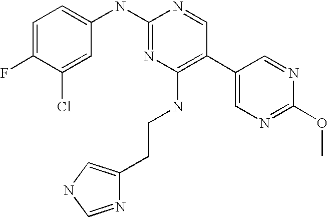 N²-(3-chloro-4-fluorophenyl)-N⁴-[2-(1H-imidazol-4- yl)ethyl]-2′-methoxy-5,5′-bipyrimidine-2,4-diamine | MS(ES): 441 (M + 1) for C₂₀H₁₈ClFN₈O. 400 MHz, DMSO-d₆: δ 2.70-2.90 (m, 2H), 3.56-3.57 (m, 2H), 3.95 (s, 3H), 6.81 (br s, 1H), 7.07 (br s, 1H), 7.25 (t, J = 9.12 Hz, 1H), 7.51 (s, 1H), 7.70 (d, J = 7.00 Hz, 1H), 7.78 (s, 1H), 8.16 (dd, J = 2.52, 6.88 Hz, 1H), 8.52 (d, J = 5.72 Hz, 2H), 9.43 (s, 1H), 11.81 (br s, 1H). | 2-(1H-Imidazol- 4-yl)-ethylamine |
| Example 463 | 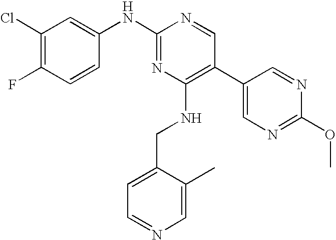 N2-(3-chloro-4-fluorophenyl)-2′-methoxy-N4-((3- methylpyridin-4-yl)methyl)-5,5′-bipyrimidine-2,4- diamine | MS(ES): 452(M + 1) for C₂₂H₁₉ClFN₇O. 400 MHz, DMSO-d₆: δ 2.29 (s, 3 H) 3.96 (s, 4 H) 4.50 (m, 2 H) 7.16 (mm, 2 H) 7.37 (mm, 2 H) 7.85 (mm, 2 H) 8.34 (mm, 2 H) 8.68 (s, 2 H) 9.37 (s, 1 H) | (3-methylpyridin- 4-yl)methanamine |
| Example 464 | 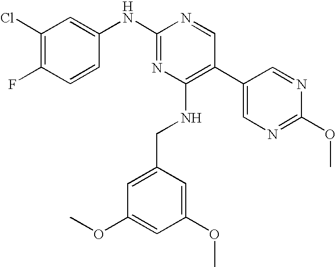 N2-(3-chloro-4-fluorophenyl)-N4-(3,5-dimethoxybenzyl)- 2′-methoxy-5,5′-bipyrimidin-2,4-diamine | MS(ES): 497(M + 1) for C₂₄H₂₂ClFN₆O₃. 400 MHz, DMSO-d₆: δ 3.66 (s, 6 H) 3.96 (s, 3 H) 4.49 (m, 2 H) 6.33 (s, 1 H) 6.46 (s, 2 H) 7.23 (t, 1 H) 7.53 (m, 1 H) 7.65 (m, 1 H) 7.83 (s, 1 H) 8.03 (m, 1 H) 8.59 (s, 2 H) 9.59 (s, 1 H) | 3,5-dimethoxy benzylamine |
| Example 465 | 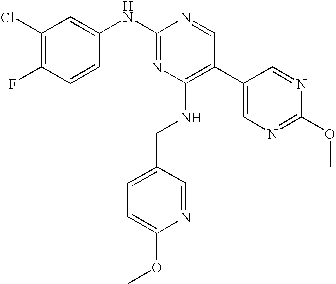 N2-(3-chloro-4-fluorophenyl)-2′-methoxy-N4-((6- methoxypyridin-3-yl)methyl)-5,5′-bipyrimidine-2,4- diamine | MS(ES): 468(M + 1) for C₂₂H₁₉ClFN₇O₂. 400 MHz, DMSO-d₆: δ 3.78 (s, 3 H) 3.95 (m, 3 H) 4.48 (m, 2 H) 6.75 (d, 1 H) 7.26 (t, 1 H) 7.55 (mm, 2 H) 7.67 (d, 1 H) 7.82 (s, 1 H) 8.07 (m, 1 H) 8.13 (s, 1 H) 8.58 (s, 2 H) 9.48 (s, 1 H) | (6- methoxypyridin- 3-yl)methanamine |
| Example 466 | 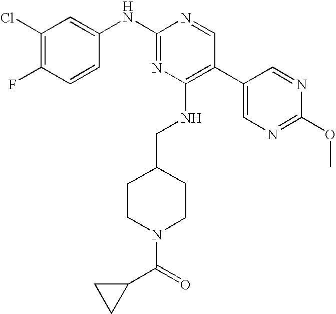 (4-((2-(3-chloro-4-fluorophenylamino)-2′-methoxy- 5,5′-bipyrimidin-4-ylamino)methyl)piperidin-1- yl)(cyclopropyl)methanone | MS(ES): 512(M + 1) for C₂₅H₂₇ClFN₇O₂. 400 MHz, DMSO-d₆: δ 0.66 (m, 4 H) 0.99 (m, 2 H) 1.75 (m, 2 H) 1.93 (m, 2 H) 3.01 (s, 1 H) 3.26 (mm, 4 H) 3.99 (s, 3 H) 4.31 (m, 2 H) 7.11 (m, 1 H) 7.29 (t, 1 H) 7.55 (m, 1 H) 7.77 (s, 1 H) 8.24 (m, 1 H) 8.55 (s, 2 H) 9.49 (s, 1 H) | (4- (aminomethyl)- piperidin-1- yl)(cyclopropyl)- methanone |
| Example 467 | 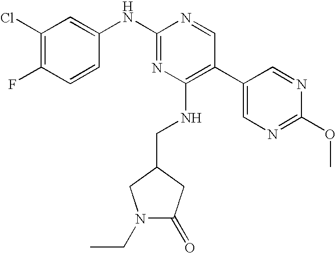 4-((2-(3-chloro-4-fluorophenylamino)-2′-methoxy-5,5′- bipyrimidin-4-ylamino)methyl)-1-ethylpyrrolidin- 2-one | MS(ES): 472(M + 1) for C₂₂H₂₃ClFN₇O₂. 400 MHz, DMSO-d₆: δ 0.96 (t, 3 H) 2.06 (m, 1 H) 2.36 (m, 1 H) 2.71 (m, 1 H) 3.14 (m, 3 H) 3.43 (m, 3 H) 3.96 (s, 3 H) 7.14 (s, 1H) 7.29 (t, 1 H) 7.56 (m, 1 H) 7.79 (s, 1 H) 8.22 (m, 1 H) 8.55 (s, 2 H) 9.48 (s, 1 H) | 4-(aminomethyl)- 1-ethylpyrrolidin- 2-one |
| Example 468 | 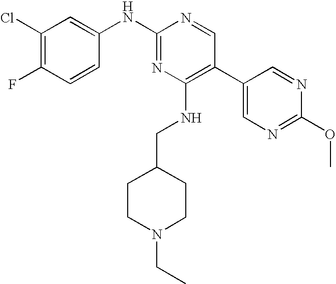 N2-(3-chloro-4-fluorophenyl)-N4-((1-ethylpiperidin-4- yl)methyl)-2′-methoxy-5,5′-bipyrimidine-2,4-diamine | MS(ES): 472(M + 1) for C₂₃H₂₇ClFN₇O. 400 MHz, DMSO-d₆: δ 0.95 (t, 3 H) 1.14 (m, 2 H) 1.65 (m, 4 H) 2.24 (q, 2 H) 2.82 (m, 2 H) 3.21 (bs, 2 H) 3.95 (s, 3 H) 6.92 (m, 1 H) 7.27 (t, 1 H) 7.58 (m, 1 H) 7.74 (s, 1 H) 8.24 (m, 1 H) 8.53 (s, 2 H) 9.41 (s, 1 H) | (1-ethylpiperidin- 4-yl)- methanamine |
| Example 469 | 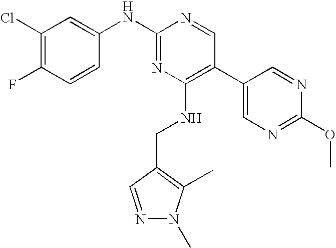 N2-(3-chloro-4-fluorophenyl)-N4-((1,5-dimethyl-1H- pyrazol-4-yl)methyl)-2′-methoxy-5,5′-bipyrimidine- 2,4-diamine | MS(ES): 455(M + 1) for C₂₁H₂₀ClFN₈O. 400 MHz, DMSO-d₆: δ 2.17 (s, 3 H) 3.64 (s, 3 H) 3.94 (s, 4 H) 4.32 (m, 2 H) 7.17 (bs, 1 H) 7.28 (m, 2 H) 7.63 (m, 1 H) 7.77 (s, 1 H) 8.17 (m, 1 H) 8.51 (m, 2 H) 9.42 (s, 1 H) | (1,5-dimethyl- 1H-pyrazol-4- yl)-methanamine |
| Example 470 | 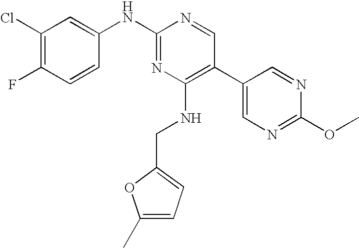 N2-(3-chloro-4-fluorophenyl)-2′-methoxy-N4-((5- methylfuran-2-yl)methyl)-5,5′-bipyrimidine-2,4-diamine | MS(ES): 441(M + 1) for C₂₁H₁₈ClFN₆O₂. 400 MHz, DMSO-d₆: δ 2.19 (s, 3 H) 3.96 (s, 3 H) 4.46 (d, 2 H) 5.96 (s, 1 H) 6.09 (s, 1 H) 7.36 (t, 1 H) 7.55 (m, 1 H) 7.88 (s, 1 H) 8.06 (m, 2 H) 8.56 (s, 2 H) 10.14 (s, 1 H) | (5-methylfuran-2- yl)methanamine |

### Example 471

### N²-(3-chloro-4-fluorophenyl)-N⁴-[2-(1H-imidazol-4-yl)ethyl]-5,5′-bipyrimidine-2,4-diamine

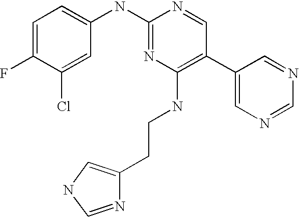

To a suspension of NaH (2.2 mmol, 60% dispersion in oil) in DMF, 2-(1H-Imidazol-4-yl)-ethylamine (1 eq) was added and stirred for 30 minutes. N-(3-chloro-4-fluorophenyl)-4-methylsulfonyl-5-pyrimidin-5-ylpyrimidin-2-amine Intermediate 123 (1 eq) in DMF was added to the reaction mixture drop wise and stirred at room temperature 16 hours. The reaction mixture was added to water and stirred for 15 min. The precipitated solid was filtered, washed with water and dried to afford the crude product. It was further purified by flash chromatography using chloroform:methanol (9:1) to get the pure product.

| Compound | Structure | Mass and ¹H NMR data | SM |
|---|---|---|---|
| Example 471 | 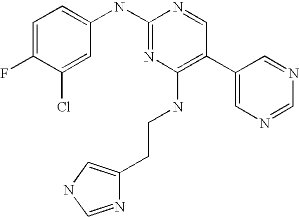 N²-(3-chloro-4-fluorophenyl)-N⁴-[2-(1H- imidazol-4-yl)ethyl]-5,5′-bipyrimidine- 2,4-diamine | MS(ES): 411 (M + 1) for C₁₉H₁₆ClFN₈. 400 MHz, DMSO-d₆: δ 2.70- 2.90 (m, 2H), 3.56-3.61 (m, 2H), 6.85 (br s, 1H), 7.07 (br s, 1H), 7.25 (t, J = 9.12 Hz, 1H), 7.51 (s, 1H), 7.72 (br s, 1H), 7.86 (s, 1H), 8.16 (dd, J = 2.52, 6.88 Hz, 1H), 8.77 (s, 2H), 9.15 (s, 1H), 9.49 (s, 1H), 11.8 (br s, 1H). | 2-(1H- Imidazol-4- yl)- ethylamine |

### General Procedure for Conversion of Pyridine Carboxylic Esters to Carboxamide Derivatives

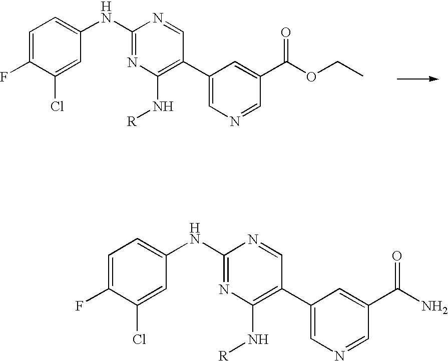

To a solution of ester (1 eq) in THF (2 mL) was added aqueous ammonia solution (20 mL) and the mixture was heated to 60° C. in a sealed tube for 16-24 h. The reaction mixture was cooled to room temperature, the solid thus obtained was filtered, washed with water and dried to give product.

Compounds in the below table were prepared using this general procedure and the specified starting material.

| Compound | Structure | Mass and ¹H NMR data | SM |
|---|---|---|---|
| Example 472 | 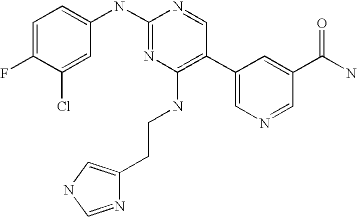 5-(2-[(3-chloro-4-fluorophenyl)amino]-4-{[2- (1H-imidazol-4-yl)ethyl]amino}pyrimidin-5-yl)- pyridine-3-carboxamide | MS(ES): 453 (M + 1) for C₂₁H₁₈ClFN₈O 400 MHz, DMSO-d₆: δ 2.80-2.83 (m, 2H), 3.59-3.64 (m, 2H), 6.83 (br s, 1H), 7.13 (br s, 1H), 7.27 (t, J = 9.20 Hz, 1H), 7.50 (s,1H), 7.65 (br s, 1H), 7.73-7.75 (m, 1H), 7.88 (s, 1H), 8.15-8.18 (m, 2H), 8.21-8.22 (m, 1H), 8.67 (d, J = 1.20 Hz, 1H), 8.98 (d, J = 2.00 Hz, 1H), 9.47 (s, 1H), 11.82 (br s, 1H). | Example 154 ethyl 5-(2-[(3- chloro-4- fluorophenyl)- amino]-4-{[2- (1H-imidazol- 4-yl)ethyl] amino}- pyrimidin-5- yl)pyridine-3- carboxylate |
| Example 473 | 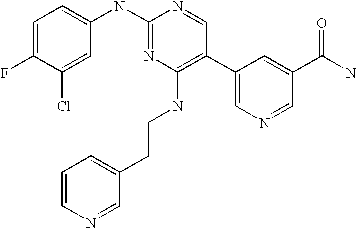 5-(2-[(3-chloro-4-fluorophenyl)amino]-4-{[2- (pyridin-3-yl)ethyl]amino}pyrimidin-5-yl)- pyridine-3-carboxamide | MS(ES): 464 (M + 1) for C₂₃H₁₉ClFN₇O 400 MHz, DMSO-d₆: δ 2.91 (t, J = 7.20 Hz, 2H), 3.61-3.63 (m, 2H), 7.26-7.33 (m, 2H), 7.62-7.66 (m, 3H), 7.88 (s, 1H), 8.16 (s, 2H), 8.21 (dd, J = 2.40, 6.80 Hz, 1H), 8.41-8.45 (m, 2H), 8.62 (d, J = 1.60 Hz, 1H), 8.98 (d, J = 2.00 Hz, 1H), 9.48 (s, 1H). | Example 102 ethyl 5-(2-[(3- chloro-4- fluorophenyl)- amino]-4-{[2- (pyridin-3- yl)ethyl]amino}- pyrimidin-5- yl)pyridine-3- carboxylate |
| Example 474 | 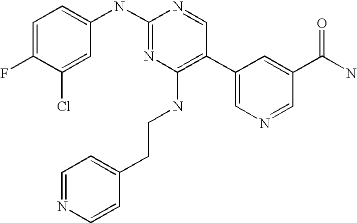 5-(2-[(3-chloro-4-fluorophenyl)amino]-4-{[2- (pyridin-4-yl)ethyl]amino}pyrimidin-5-yl)- pyridine-3-carboxamide | MS(ES): 464 (M + 1) for C₂₃H₁₉ClFN₇O. 400 MHz, DMSO-d₆: δ 2.99 (t, J = 7.00 Hz, 2H), 3.75 (t, J = 7.20 Hz, 2H), 7.15 (t, J = 9.00 Hz, 1H), 7.28 (dd, J = 1.44, 4.60 Hz, 2H), 7.47 (ddd, J = 2.72, 4.10, 8.99 Hz, 1H), 7.81 (s, 1H), 8.07 (dd, J = 2.64, 6.74 Hz, 1H), 8.23- 8.24 (m, 1H), 8.39 (dd, J = 1.52, 4.56 Hz, 2H), 8.65 (d, J = 2.12 Hz, 1H), 8.98 (d, J = 2.12 Hz, 1H). | Example 103 ethyl 5-(2-[(3- chloro-4- fluorophenyl)- amino]-4-{[2- (pyridin-4- yl)ethyl]amino}- pyrimidin-5- yl)pyridine-3- carboxylate |

Synthesis of N-Methoxy Carboxamides from Carboxylic Acids

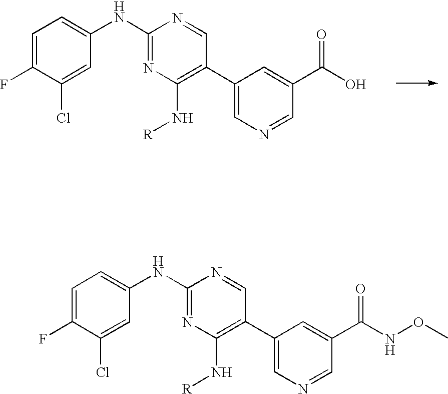

### Method A:

To a mixture of (Example 257) (1 eq), triethylamine (3 eq) and methoxylamine hydrochloride (1.2 eq) in DMF was added HOBt (5 mol %), EDCI (1.2 eq) slowly at 0° C. The reaction mixture was slowly raised to room temperature and stirred for overnight. Water was added and the precipitate thus formed was filtered and dried to yield Example 475.

### Method B:

To a mixture of (Example 229) (1 eq), triethylamine (4 eq) and methoxylamine hydrochloride (1.2 eq) in EtOAc:DCM (1:1) was added T3P (50%, 1.5 eq) slowly at 0° C. The reaction mixture was slowly raised to room temperature and stirred for overnight. Reaction mixture was then diluted with dichloromethane (12 mL), washed the dichloromethane solution successively with water (2×50 mL), 10% aq sodium bicarbonate solution (50 mL) and brine. The organic layer was dried over sodium sulphate, filtered, concentrated and dried to yield Example 476.

### Method C:

To a mixture of (Example 230) (1 eq), triethylamine (3.5 eq) and methoxylamine hydrochloride (1.0 eq) in DMF was added BOP (1.2 eq) slowly at 0° C. The reaction mixture was slowly raised to room temperature and stirred for 3-4 h. Water was added followed by extraction with EtOAc. The organic layer was dried over sodium sulphate, filtered and concentrated. The resulting residue was purified by silica gel column chromatography using chloroform:methanol (9:1) as an eluent to afford Example 477.

The compounds in the below table were prepared following the methods described above as indicated with the starting material listed.

| Compound | Structure | Mass and ¹H NMR data | SM |
|---|---|---|---|
| Example 475^{a} | 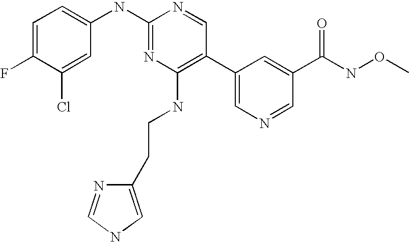 5-(2-[(3-chloro-4-fluorophenyl)amino]-4-{[2-(1H-imidazol-4- yl)ethyl]amino}pyrimidin-5-yl)-N-methoxypyridine-3-carboxamide | MS (ES): 481 (M − 1) for C₂₂H₂₀ClFN₈O₂. 400 MHz, DMSO-d₆: δ 2.80 (br s, 2H), 3.60-3.61 (m, 2H), 3.74 (s, 3H), 6.84 (br s, 1H), 7.13 (br s, 1H), 7.26 (t, J = 9.44 Hz, 1H), 7.50 (s, 1H), 7.73 (br s, 1H), 7.87 (s, 1H), 8.09 (s, 1H), 8.15 (d, J = 6.24 Hz, 1H), 8.69 (s, 1H), 8.86 (s, 1H), 9.48 (s, 1H), 12.00 (br s, 2H). | Example 527 5-(2-[(3- chloro-4- fluorophenyl)- amino]-4-{[2- (1H-imidazol- 4- yl)ethyl]amino}- pyrimidin-5- yl)pyridine-3- carboxylic acid |
| Example 476^{b} | 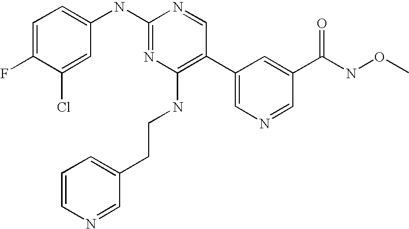 5-(2-[(3-chloro-4-fluorophenyl)amino]-4-{[2-(pyridin-3- yl)ethyl]amino}pyrimidin-5-yl)-N-methoxypyridine- 3-carboxamide | MS(ES): 492 (M − 1) for C₂₄H₂₁ClFN₇O₂. 400 MHz, DMSO-d₆: δ 2.91- 2.93 (m, 2H), 3.61-3.63 (m, 2H), 3.76 (s, 3H), 7.04 (t, J = 5.20 Hz, 1H), 7.27-7.33 (m, 2H), 7.62-7.65 (m, 2H), 7.88 (s, 1H), 8.03 (s, 1H), 8.21 (dd, J = 1.20, 7.40 Hz, 1H), 8.43 (br s, 2H), 8.65 (s, 1H), 8.87 (s, 1H), 9.49 (s, 1H), 11.96 (br s, 1H). | Example 229 5-(2-[(3- chloro-4- fluorophenyl)- amino]-4-{[2- (pyridin-3- yl)ethyl]amino}- pyrimidin-5- yl)pyridine-3- carboxylic acid |
| Example 477^{c} | 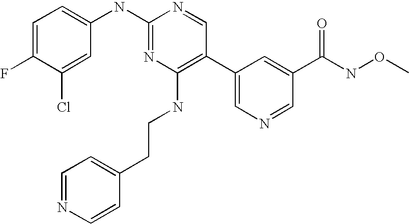 5-(2-[(3-chloro-4-fluorophenyl)amino]-4-{[2-(pyridin-4- yl)ethyl]amino}pyrimidin-5-yl)-N-methoxypyridine- 3-carboxamide | MS(ES): 494 (M + 1) for C₂₄H₂₁ClFN₇O₂. 400 MHz, DMSO-d₆: δ 2.90 (t, J = 6.72 Hz, 2H), 3.60-3.65 (m, 2H), 3.74 (s, 3H), 7.02 (t, J = 5.68 Hz, 1H), 7.23 (d, J = 5.60 Hz, 2H), 7.27 (t, J = 9.08 Hz, 1H), 7.59-7.63 (m, 1H), 7.87 (s, 1H), 8.03 (s, 1H), 8.19 (dd, J = 2.56, 6.86 Hz, 1H), 8.45 (d, J = 5.76 Hz, 2H), 8.64 (s, 1H), 8.86 (d, J = 1.72 Hz, 1H), 9.48 (s, 1H), 11.96 (br s, 1H). | Example 230 5-(2-[(3- chloro-4- fluorophenyl)- amino]-4-{[2- (pyridin-4- yl)ethyl]amino}- pyrimidin-5- yl)pyridine-3- carboxylic acid |

^{a}Method A
^{b}Method B
^{c}Method C

### General Procedure for Aryl-Aryl Coupling Reaction using Intermediate 146

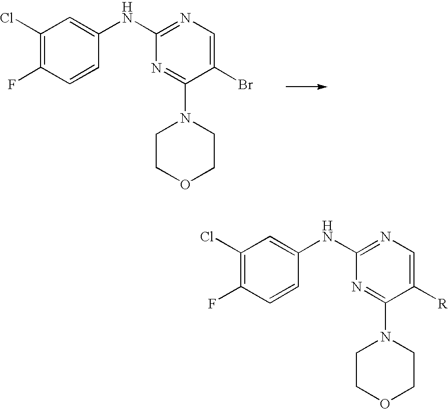

A suspension of Intermediate 146 (1 eq), boronic acid (1.05 eq), tris(dibenzylideneacetone)dipalladium(0) (10 mol %), 2-dicyclohexylphosphino-2′,4′,6′-triisopropyl-1,1′-biphenyl (30 mol %) and sodium carbonate (2 eq) in acetonitrile/water (4:1) was heated to 90° C. for 30 minutes. The reaction mixture was diluted with ethyl acetate; organic layer was separated, dried over sodium sulfate, filtered and concentrated. The resulting residue was purified by silica gel column chromatography using chloroform:methanol (9:1) as an eluent to afford the product.

The compounds in the below table were prepared using this general method and the specified starting material.

| Compound | Structure | Mass spectrum and ¹H NMR | SM |
|---|---|---|---|
| Example 478 | 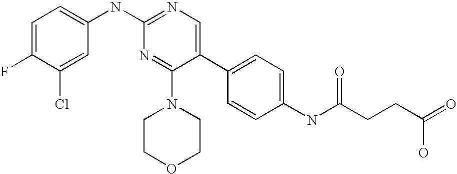 4-[(4-{2-[(3-chloro-4-fluorophenyl)amino]-4-(morpholin-4- yl)pyrimidin-5-yl}phenyl)amino]-4-oxobutanoic acid | MS(ES): 500 (M + 1) for C₂₄H₁₃ClFN₅O₄. 400 MHz, AcOH : δ 1.42 (s, 4H), 2.03-2.07 (m, 4H), 2.37 (t, J = 4.96 Hz, 4H), 5.88 (t, J = 9.00 Hz, 1H), 6.13 (d, J = 8.52 Hz, 2H), 6.20 (dd, J = 2.72, 4.02, 8.95 Hz, 1H), 6.38 (d, J = 8.60 Hz, 2H), 6.66 (br s, 1H), 6.78 (dd, J = 2.68, 6.74 Hz, 1H). | 4-oxo-4- {[4-(4,4,5,5- tetramethyl- 1,3,2- dioxaborolan- 2-yl)phenyl]- amino}- butanoic acid |
| Example 479 | 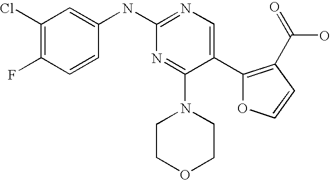 2-{2-[(3-chloro-4-fluorophenyl)amino]-4-(morpholin-4- yl)pyrimidin-5-yl}furan-3-carboxylic acid | MS(ES): 419 (M + 1) for C₁₉H₁₆ClFN₄O₄. 400 MHz, DMSO-d₆ : δ 3.24 (t, J = 4.56 Hz, 4H), 3.57 (t, J = 4.92 Hz, 4H), 6.80 (d, J = 1.92 Hz, 1H), 7.33 (t, J = 9.12 Hz, 1H), 7.61 (ddd, J = 2.76, 4.20, 9.05 Hz, 1H), 7.78 (d, J = 1.84 Hz, 1H), 8.07 (dd, J = 5.48, 5.44 Hz, 1H), 8.10 (s, 1H), 9.68 (s, 1H). | 2- (dihydroxy- boranyl)furan- 3- carboxylic acid |
| Example 480 | 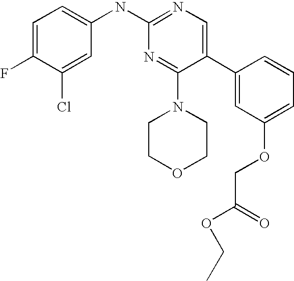 ethyl (3-{2-[(3-chloro-4-fluorophenyl)amino]-4- (morpholin-4-yl)pyrimidin-5-yl}phenoxy)acetate | MS(ES): 487 (M + 1) for C₂₄H₂₄ClFN₄O₄. 400 MHz, DMSO-d₆ : δ 1.20 (t, J = 7.08 Hz, 3H), 3.22 (t, J = 4.32 Hz, 4H), 3.55 (t, J = 4.84 Hz, 4H), 4.17 (q, J = 7.08 Hz, 2H), 4.82 (s, 2H), 6.87 (dd, J = 2.48, 8.04 Hz, 1H), 6.99 (t, J = 1.48 Hz, 1H), 7.06 (d, J = 7.72 Hz, 1H), 7.29-7.36 (m, 2H), 7.61 (ddd, J = 2.68, 4.16, 9.09 Hz, 1H), 8.01 (s, 1H), 8.14 (dd, J = 2.64, 6.88 Hz, 1H), 9.60 (s, 1H). | ethyl [3- (4,4,5,5- tetramethyl- 1,3,2- dioxaborolan- 2- yl)phenoxy] acetate |
| Example 481 | 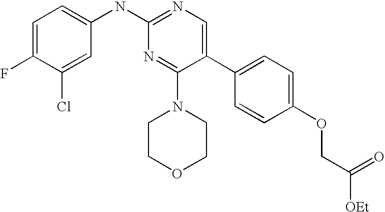 ethyl (4-{2-[(3-chloro-4-fluorophenyl)amino]-4- (morpholin-4-yl)pyrimidin-5-yl}phenoxy)acetate | MS(ES): 459 (M + 1) for C₂₂H₂₀ClFN₄O₄. 400 MHz, DMSO-d₆: δ 1.21 (t, J = 7.08 Hz, 3H), 3.22 (d, J = 3.88 Hz, 4H), 3.55 (t, J = 4.12 Hz, 4H), 4.17 (q, J = 7.04 Hz, 2H), 4.80 (s, 2H), 6.99 (d, J = 8.48 Hz, 2H), 7.31 (t, J = 9.04 Hz, 1H), 7.38 (d, J = 8.44 Hz, 2H), 7.60- 7.63 (m, 1H), 7.97 (s, 1H), 8.14 (dd, J = 2.28, 6.80 Hz, 1H), 9.55 (s, 1H). | ethyl [4- (4,4,5,5- tetramethyl- 1,3,2- dioxaborolan- 2- yl)phenoxy] acetate |
| Example 482 | 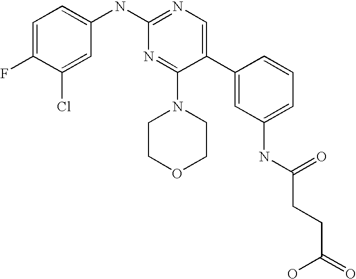 4-[(3-{2-[(3-chloro-4-fluorophenyl)amino]-4-(morpholin- 4-yl)pyrimidin-5-yl}phenyl)amino]-4-oxobutanoic acid | MS(ES): 500 (M + 1) for C₂₄H₂₃ClFN₅O₄. 400 MHz, DMSO-d₆ : δ 2.49-2.53 (m, 2H), 2.55-2.57 (m, 2H), 3.22-3.26 (m, 4H), 3.54-3.58 (m, 4H), 7.11 (d, J = 7.56 Hz, 1H), 7.26-7.36 (m, 2H), 7.51 (d, J = 8.36 Hz, 1H), 7.60-7.63 (m, 1H), 7.72 (s, 1H), 7.97 (s, 1H), 8.13 (d, J = 6.76 Hz, 1H), 9.59 (s, 1H), 10.07 (s, 1H), 12.16 (br s, 1H). | 4-oxo-4- {[3-(4,4,5,5- tetramethyl- 1,3,2- dioxaborolan- 2-yl)phenyl]- amino}- butanoic acid |
| Example 483 | 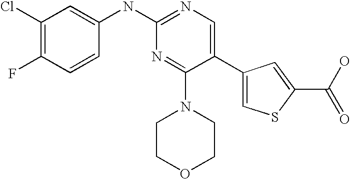 4-{2-[(3-chloro-4-fluorophenyl)amino]-4-(morpholin-4- yl)pyrimidin-5-yl}thiophene-2-carboxylic acid | MS(ES): 433 (M − 1) for C₁₉H₁₆ClFN₄O₃S. 400 MHz, DMSO-d₆ : δ 3.25 (t, J = 4.12 Hz, 4H), 3.55-3.60 (m, 4H), 7.32 (t, J = 9.04 Hz, 1H), 7.62 (dd, J = 3.04, 8.80 Hz, 1H), 7.88 (s, 1H), 7.92 (s, 1H), 8.12-8.17 (m, 1H), 8.31 (s, 1H), 9.66 (s, 1H), 13.30 (br s, 1H). | 4-(4,4,5,5- tetramethyl- 1,3,2- dioxaborolan- 2- yl)thiophene- 2-carboxylic acid |
| Example 484 | 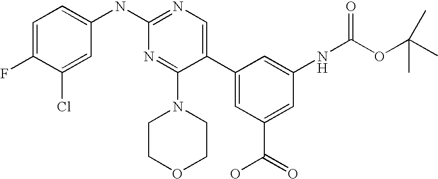 3-[(tert-butoxycarbonyl)amino]-5-{2-[(3-chloro-4- fluorophenyl)amino]-4-(morpholin-4-yl)pyrimidin-5- yl}benzoic acid | MS(ES): 544 (M + 1) for C₂₆H₂₇ClFN₅O₅. 400 MHz, DMSO-d₆: δ 1.49 (s, 9H), 3.23 (br s, 4H), 3.57 (br s, 4H), 7.33 (t, J = 9.12 Hz, 1H), 7.62-7.64 (m, 2H), 7.76 (br s, 1H), 8.02 (s, 1H), 8.10 (s, 1H), 8.13 (dd, J = 2.52, 6.92 Hz, 1H), 9.67 (dd, J = 8.76, Hz, 2H), 13.03 (br s, 1H). | 3-[(tert- butocy- carbonyl)- amino]-5- (dihydroxy- boranyl)- benzoic acid |
| Example 485 | 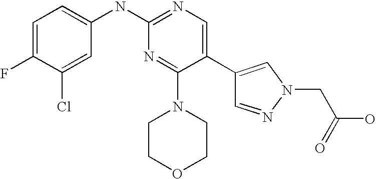 (4-{2-[(3-chloro-4-fluorophenyl)amino]-4-(morpholin-4- yl)pyrimidin-5-yl}-1H-pyrazol-1-yl)acetic acid | MS(ES): 433 (M + 1) for C₁₉H₁₈ClFN₆O₃. 400 MHz, DMSO-d₆ : δ 3.26 (t, J = 4.24 Hz, 4H), 3.65 (t, J = 4.72 Hz, 4H), 4.98 (s, 2H), 7.30 (t, J = 9.12 Hz, 1H), 7.59- 7.63 (m, 1H), 7.70 (s, 1H), 7.97 (s, 1H), 8.13 (s, 1H), 8.15 (d, J = 2.60 Hz, 1H), 9.57 (s, 1H), 13.08 (br s, 1H). | ethyl [4- (4,4,5,5- tetramethyl- 1,3,2- dioxaborolan- 2-yl)-1H- pyrazol-1- yl]acetate |
| Example 486 | 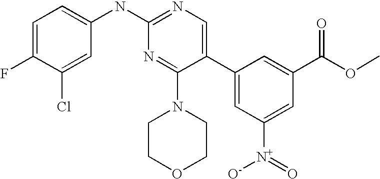 methyl 3-{2-[(3-chloro-4-fluorophenyl)amino]-4- (morpholin-4-yl)pyrimidin-5-yl}-5-nitrobenzoate | MS(ES)P: 488.2 (M + 1) for C₂₂H₁₉ClFN₅O₅. 400 MHz, DMSO-d₆ : δ 3.22 (t, J = 4.08 Hz, 4H), 3.55 (t, J = 4.52 Hz, 4H), 3.94 (s, 3H), 7.34 (t, J = 9.12 Hz, 1H), 7.63 (ddd, J = 2.76, 4.16, 9.09 Hz, 1H), 8.13 (dd, J = 2.60, 6.88 Hz, 1H), 8.23 (s, 1H), 8.44 (t, J = 1.48 Hz, 1H), 8.51 (t, J = 2.08 Hz, 1H), 8.56 (t, J = 1.76 Hz, 1H), 9.80 (s, 1H). | methyl 3- nitro-5- (4,4,5,5- tetramethyl- 1,3,2- dioxaborolan- 2- yl)benzoate |
| Example 487 | 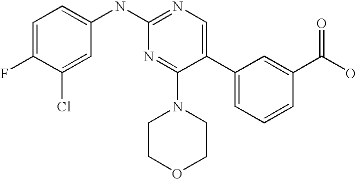 3-{2-[(3-chloro-4-fluorophenyl)amino]-4-(morpholin- 4-yl)pyrimidin-5-yl}benzoic acid | MS(ES): 429 (M + 1) for C₂₁H₁₈ClFN₄O₃. 400 MHz, DMSO-d₆: δ 3.21 (br s, 4H), 3.55 (br s, 4H), 7.32 (t, J = 9.04 Hz, 1H), 7.56 (t, J = 7.36 Hz, 1H), 7.62-7.64 (m, 1H), 7.71 (d, J = 7.68 hz, 1H), 7.87 (d, J = 7.84 Hz, 1H), 8.04 (br s, 1H), 8.08 (br s, 1H), 8.14 (d, J = 4.88 Hz, 1H), 9.65 (s, 1H), 13.12 (br s, 1H). | 3-(dihydroxy- boranyl)- benzoic acid |

### General Procedure for Hydrolysis of Carboxylic Acid Ester Derivatives

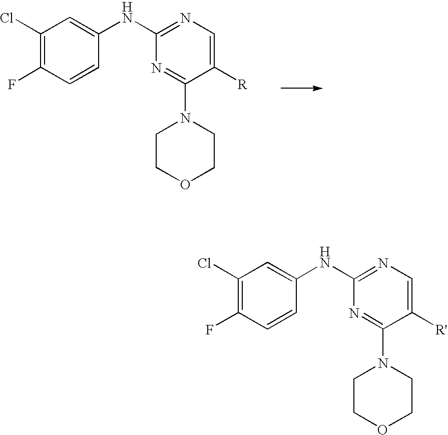

Starting ester (1 eq, 0.22 mmol) was dissolved in a mixture of tetrahydrofuran (1 mL) and water (1 mL) and treated with 1 N sodium hydroxide (4 eq, 0.88 mmol) and allowed to stir at room temperature for 16 hours. Reaction mixture was then carefully acidified with 1 N HCl and the precipitate formed was filtered, washed with water and dried to yield the product.

The compounds in the below table were prepared using this general method and the specified starting material.

| Compound | Structure | Mass spectrum and ¹H NMR | SM |
|---|---|---|---|
| Example 488 | 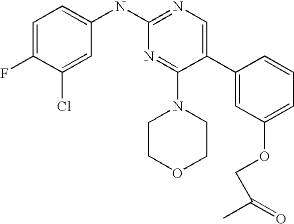 (3-{2-[(3-chloro-4-fluorophenyl)amino]-4- (morpholin-4-yl)pyrimidin-5-yl}phenoxy)acetic acid | MS(ES): 459 (M + 1) for C₂₂H₂₀ClFN₄O₄. 400 MHz, DMSO-d₆: δ 3.20- 3.25 (m, 4H), 3.55-3.60 (m, 4H), 4.43 (s, 2H), 6.80 (d, J = 7.64 Hz, 1H), 6.91 (s, 1H), 6.98 (d, J = 7.64 Hz, 1H), 7.27-7.33 (m, 2H), 7.61-7.64 (m, 1H), 7.99 (s, 1H), 8.13 (dd, J = 2.32, 6.88 Hz, 1H), 9.58 (s, 1H). | Example 480 ethyl (3-{2- [(3-chloro-4- fluorophenyl)- amino]-4- (morpholin-4- yl)pyrimidin- 5-yl}phenoxy) acetate |
| Example 489 | 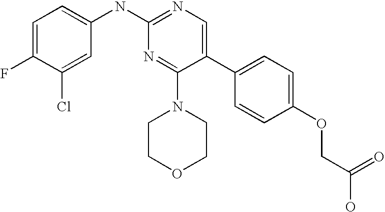 (4-{2-[(3-chloro-4-fluorophenyl)amino]-4- (morpholin-4-yl)pyrimidin-5-yl}phenoxy)acetic acid | MS(ES): 459.2 (M + 1) for C₂₂H₂₀ClFN₄O₄. 400 MHz, DMSO-d₆: δ 3.25 (br s, 4H), 3.55 (br s, 4H), 4.70 (s, 2H), 6.98 (d, J = 7.88 Hz, 2H), 7.31-7.38 (m, 3H), 7.55-7.65 (m, 1H), 7.96 (s, 1H), 8.09 (d, J = 4.92 Hz, 2H), 9.71 (s, 1H), 13.04 (br s, 1H). | Example 481 ethyl (4-{2- [(3-chloro-4- fluorophenyl)- amino]-4- (morpholin-4- yl)pyrimidin-5- yl}phenoxy) acetate |
| Example 490 | 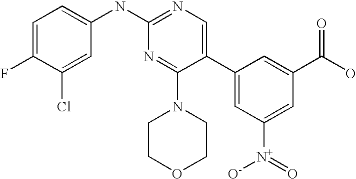 3-{2-[(3-chloro-4-fluorophenyl)amino]-4- (morpholin-4-yl)pyrimidin-5-yl}-5-nitrobenzoic acid | MS(ES): 474 (M + 1) for C₂₁H₁₇ClFN₅O_{5.} 400 MHz, DMSO-d₆: δ 3.23 (br s, 4H), 3.56 (br s, 4H), 7.33 (d, J = 9.04 Hz, 1H), 7.63-7.65 (m, 1H), 8.14 (d, J = 6.64 Hz, 1H), 8.20 (s, 1H), 8.41 (br s, 2H), 8.52 (br s, 1H), 9.76 (br s, 1H). | Example 486 methyl 3-{2- [(3-chloro-4- fluorophenyl) amino]-4- (morpholin- 4-yl) pyrimidin-5- yl}-5- nitrobenzoate |

### Example 491

### 3-amino-5-{2-[(3-chloro-4-fluorophenyl)amino]-4-(morpholin-4-yl)pyrimidin-5-yl}benzoic acid

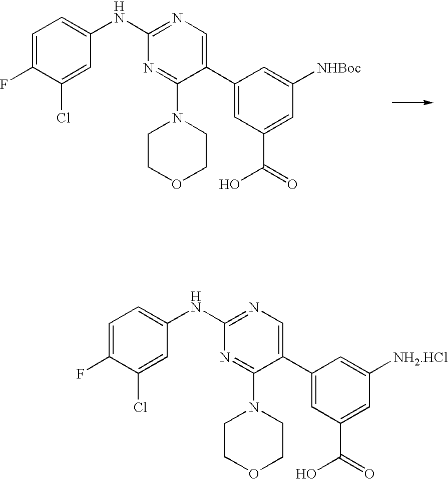

To 3-(tert-butoxycarbonylamino)-5-(2-(3-chloro-4-fluorophenylamino)-4-morpholinopyrimidin-5-yl)benzoic acid, Example 484 (0.25 mmol, 110 mg), HCl in dioxane (4 mL) was added and the mixture was stirred at room temperature for 16 hours. The solvent was evaporated and the residue was triturated with diethyl ether. The solid thus obtained was dried to yield the title compound.

MS (ES): 444 (M+1) for C₂₁H₁₉ClFN₅O₃.

400 MHz, DMSO-d₆: δ 3.38 (br s, 4H), 3.58 (br s, 4H), 7.09 (s, 1H), 7.40-7.45 (m, 3H), 7.52-7.56 (m, 1H), 7.95 (dd, J=2.32, 6.66 Hz, 1H), 7.99 (s, 1H), 10.50 (br s, 1H).

### Example 492

### methyl 4-{2-[(3-chloro-4-fluorophenyl)amino]-4-(morpholin-4-yl)pyrimidin-5-yl}pyridine-2-carboxylate

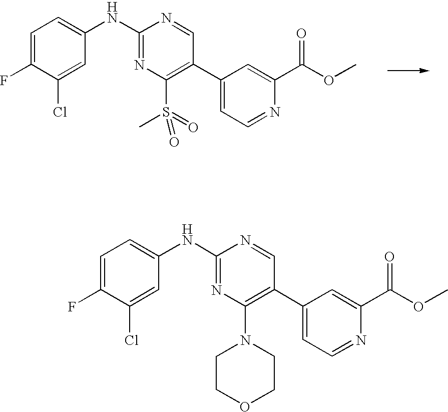

To a mixture of methyl 4-{2-[(3-chloro-4-fluorophenyl)amino]-4-(methylsulfonyl)pyrimidin-5-yl}pyridine-2-carboxylate (Intermediate 157), 0.23 mmol, 0.1 g) and morpholine (0.28 mmol, 24 mg) in NMP (2.5 mL), was added DIPEA (0.28 mmol, 36 mg) and heated to 90° C. for 1 h. Water (3 mL) was added to the reaction mixture and the solid thus formed was filtered and dried to yield the title compound.

MS(ES): 444 (M+1) for C₂₁H₁₉ClFN₅O₃

400 MHz, DMSO-d₆: δ 3.24 (t, J=4.16 Hz, 4H), 3.59 (t, J=4.20 Hz, 4H), 3.90 (s, 3H), 7.34 (t, J=9.08 Hz, 1H), 7.61-7.62 (m, 1H), 7.77 (d, J=3.60 Hz, 1H), 8.13 (dd, J=2.52, 6.68 Hz, 1H), 8.17 (br s, 1H), 8.24 (s, 1H), 8.72 (d, J=5.00 Hz, 1H), 9.81 (br s, 1H).

### Example 493

### 4-{2-[(3-chloro-4-fluorophenyl)amino]-4-(morpholin-4-yl)pyrimidin-5-yl}pyridine-2-carboxylic acid

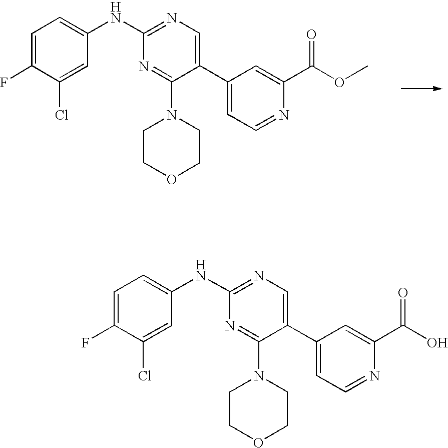

Methyl 4-{2-[(3-chloro-4-fluorophenyl)amino]-4-(morpholin-4-yl)pyrimidin-5-yl}pyridine-2-carboxylate Example 492, 0.23 mmol, 0.1 g) was dissolved in tetrahydrofuran (1 mL) and treated with aq. 1 N sodium hydroxide (0.11 mmol, 4 mg) and allowed to stir at room temperature for 1 h. After completion of the reaction, the mixture was then carefully acidified with 1 N HCl and the precipitate formed was filtered, washed with water and dried to yield the title compound.

MS(ES): 428 (M−1) for C₂₀H₁₇ClFN₅O₃.

400 MHz, DMSO-d₆: δ 3.25 (br s, 4H), 3.60 (br s, 4H), 7.35 (t, J=9.20 Hz, 1H), 7.63-7.65 (m, 1H), 7.75 (d, J=3.60 Hz, 1H), 8.14 (dd, J=2.00, 6.40 Hz, 1H), 8.18 (s, 1H), 8.24 (s, 1H), 8.71 (s, 1H), 9.81 (br s, 1H).

### Example 494

### methyl 4-{2-[(3-chloro-4-fluorophenyl)amino]-4-[3-(trifluoromethyl)-1H-pyrazol-1-yl]pyrimidin-5-yl}pyridine-2-carboxylate

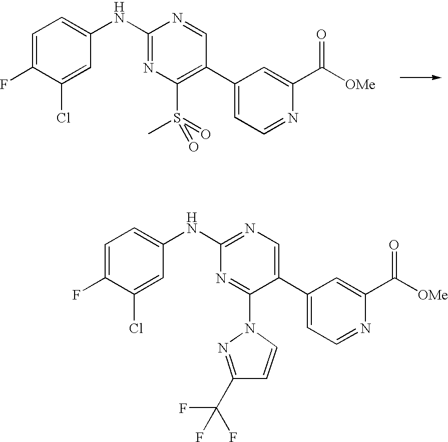

A suspension of sodium hydride (2 eq, 0.23 mmol, 5.5 mg) in NMP was cooled to 0° C. and a solution of 3-(trifluoromethyl)-1H-pyrazole (2.2 eq, 0.13 mmol, 17 mg) in NMP (1 mL) was added slowly and the reaction mixture was gradually allowed to attain room temperature. The mixture was then stirred for 30 min at room temperature. It was recooled to 0° C. and a solution of methyl 4-{2-[(3-chloro-4-fluorophenyl)amino]-4-(methylsulfonyl)pyrimidin-5-yl}pyridine-2-carboxylate (Intermediate 157, 0.114 mmol, 50 mg) in DMSO (1 mL) was added slowly to the reaction mixture and stirred for 4 h. The reaction mixture was poured into ice-water (6 mL), and filtered and dried to yield the title compound.

MS(ES): 493 (M+1) for C₂₁H₁₃ClF₄N₆O₂.

400 MHz, DMSO-d₆: δ 3.85 (s, 3H), 7.08 (d, J=2.40 Hz, 1H), 7.44 (t, J=9.20 Hz, 1H), 7.52 (br s, 1H), 7.71-7.75 (m, 1H), 7.76 (s, 1H), 8.07 (dd, J=2.40, 6.80 Hz, 1H), 8.55 (s, 1H), 8.68 (d, J=5.20 Hz, 1H), 8.85 (s, 1H), 10.54 (br s, 1H).

### Example 495

### 4-{2-[(3-chloro-4-fluorophenyl)amino]-4-[3-(trifluoromethyl)-1H-pyrazol-1-yl]pyrimidin-5-yl}pyridine-2-carboxylic acid

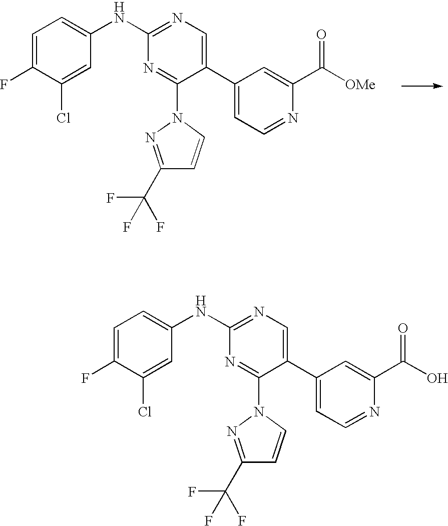

To methyl 4-{2-[(3-chloro-4-fluorophenyl)amino]-4-[3-(trifluoromethyl)-1H-pyrazol-1-yl]pyrimidin-5-yl}pyridine-2-carboxylate Example 494 (0.32 mmol, 0.16 g) taken in tetrahydrofuran (1 mL) and water (1 mL), was added Lithium hydroxide monohydrate (0.64 mmol, 28 mg) at 0° C. and was gradually allowed to attain room temperature over a period of 1 h. After completion of the reaction, the mixture was then carefully acidified with 1 N HCl and the precipitate formed was filtered, washed with water and dried to yield the title compound.

MS(ES): 479 (M+1) for C₂₀H₁₁ClF₄N₆O₂ (Taken to the next step on the basis of LCMS).

### Example 496

### 4-{2-[(3-chloro-4-fluorophenyl)amino]-4-[3-(trifluoromethyl)-1H-pyrazol-1-yl]pyrimidin-5-yl}pyridine-2-carboxamide

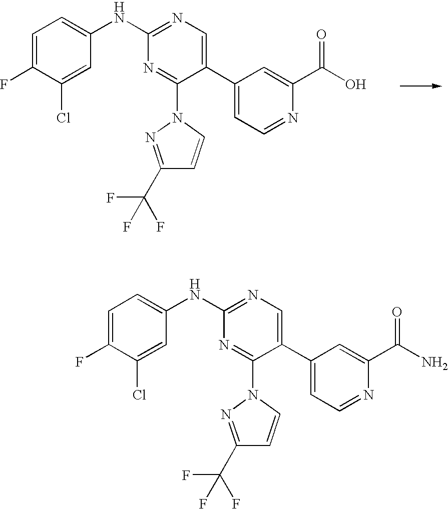

To a solution of 4-{2-[(3-chloro-4-fluorophenyl)amino]-4-[3-(trifluoromethyl)-1H-pyrazol-1-yl]pyrimidin-5-yl}pyridine-2-carboxylic acid, Example 495 (0.38 mmol, 180 mg, 1 eq), pyridine (0.38 mmol, 30 mg, 1 eq) and di-tert-butyl dicarbonate (0.48 mmol, 107 mg, 1.3 eq) in DMSO (5 mL), was added ammonium hydrogencarbonate (0.48 mmol, 39 mg 1.26 eq) and the mixture stirred for 24 h at ambient temperature. After completion of the reaction, the reaction mixture was poured into crushed ice. The solid that was formed was filtered off and further purified by column chromatography.

MS(ES): 478 (M+1) for C₂₀H₁₂ClF₄N₇O.

### Example 497

### 5-(1H-benzimidazol-2-yl)-N-(3-chloro-4-fluorophenyl)-4-(morpholin-4-yl)pyrimidin-2-amine

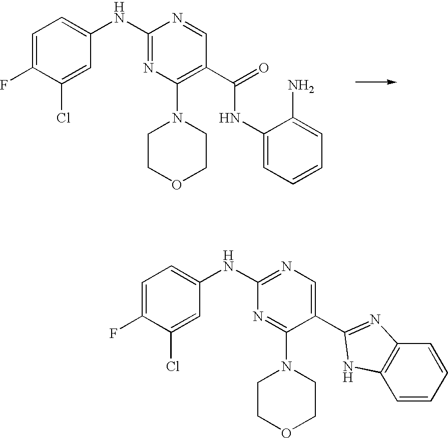

N-(2-aminophenyl)-2-[(3-chloro-4-fluorophenyl)amino]-4-(morpholin-4-yl)pyrimidine-5-carboxamide Intermediate 161 (0.16 mmol, 70 mg) was dissolved in acetic acid (3 mL) and the reaction mixture was heated at 90° C. for 8 h. The solid that had precipitated out was filtered. The filtrate was basified with NaOH and extracted with EtOAc. The organic layer was washed with water, brine, dried over Na₂SO₄ and further purified by column chromatography using methanol:chloroform (2:98) to yield 10 mg of the title compound as a white solid.

| | | Mass spectrum and ¹H | |
|---|---|---|---|
| Compound | Structure | NMR | SM |
| Example 497 | 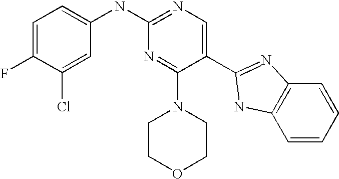 5-(1H-benzimidazol-2-yl)-N-(3-chloro-4- fluorophenyl)-4-(morpholin-4-yl)pyrimidin- 2- amine | MS(ES): 425 (M + 1) for C₂₁H₁₈ClFN₆O. 400 MHz, DMSO-d₆: δ 3.29- 3.32 (m, 4H), 3.60-3.63 (m, 4H), 7.14-7.21 (m, 2H), 7.35 (t, J = 9.08 Hz,1H),7.48- 7.50 (m, 1H), 7.61-7.64 (m, 2H), 8.11 (dd, J = 2.40, 6.90 Hz, 1H), 8.38 (s, 1H), 9.78 (s, 1H), 12.49 (s, 1H). | Intermediate 161 N-(2- aminophenyl)- 2-[(3-chloro-4- fluorophenyl)- amino]-4- (morpholin-4- yl)pyrimidine- 5-carboxamide |

### Example 498

### 4-(2-{2-[(3-chloro-4-fluorophenyl)amino]-4-[3-methoxypropyl)amino]pyrimidin-5-yl}-1,3-thiazol-4-yl)benzonitrile

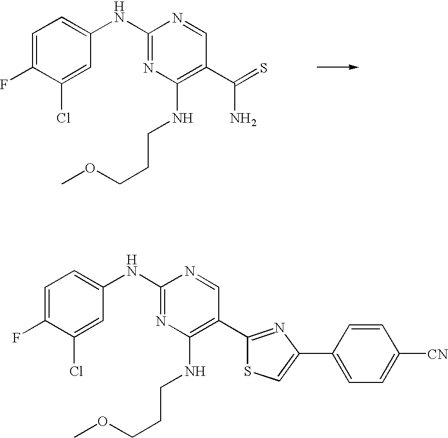

To a suspension of 2-[(3-chloro-4-fluorophenyl)amino]-4-[(3-methoxypropyl)amino]pyrimidine-5-carbothioamide (Intermediate 159, 0.14 mmol, 50 mg) and magnesium sulphate (0.14 mmol, 16 mg) in dry acetone (2 mL) under nitrogen atmosphere, was added 4-cyanophenacyl bromide (0.14 mmol, 33 mg). The resulting mixture was stirred at reflux temperature for 3 h, concentrated and purified by silica gel column chromatography (60-120 mesh) using ethyl acetate/hexanes as eluent to afford the title compound (23 mg).

| | | Mass spectrum and ¹H | |
|---|---|---|---|
| Compound | Structure | NMR | SM |
| Example 498 | 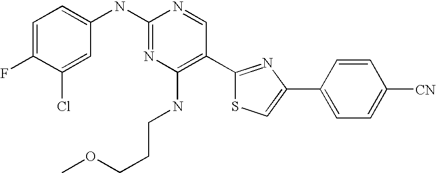 4-(2-{2-[(3-chloro-4-fluorophenyl)amino]-4-[(3- methoxypropyl)amino]pyrimidin-5-yl}- 1,3-thiazol-4-yl)benzonitrile | MS(ES): 495 (M + 1) for C₂₄H₂₀ClFN₆OS 400 MHz, DMSO-d₆: δ 1.96 (t, J = 6.28 Hz, 2H), 3.22 (s, 3H), 3.49 (t, J = 6.00 Hz, 2H), 3.69 (q, J = 6.40 Hz, 2H), 7.33 (t, J = 9.08 Hz, 1H), 7.63-7.64 (m, 1H), 7.95 (d, J = 8.40 Hz, 2H), 8.17 (d, J = 8.40 Hz, 2H), 8.25 (dd, J = 2.40, 6.80 Hz, 1H), 8.32 (s, 1H), 8.59 (s, 1H), 9.27 (t, J = 4.40 Hz, 1H), 9.86 (br s, 1H). | 4-cyanophenacyl bromide and Intermediate 159 |

### Example 499

### N²-(3-chloro-4-fluorophenyl)-N⁴-(3-methoxypropyl)-5-[4-(pyridin-3-yl)-1,3-thiazol-2-yl]pyrimidine-2,4-diamine

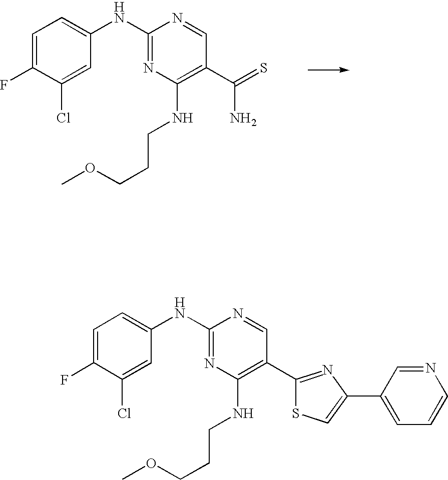

To a solution of 2-[(3-chloro-4-fluorophenyl)amino]-4-[(3-methoxypropyl)amino]pyrimidine-5-carbothioamide (Intermediate 159), 0.54 mmol, 200 mg) in ethanol (2 mL) was added 3-bromoacetylpyridine hydrobromide (0.59 mmol, 0.167 g) and triethylamine (0.5 mmol, 50 mg). The resulting mixture was subjected to microwave irradiation at 150° C. for 2 h. The precipitated solid was filtered, washed with water and dried to afford the crude product. It was further purified by silica gel column chromatography (60-120 mesh) to afford the title compound (40 mg).

| | | Mass spectrum and ¹H | |
|---|---|---|---|
| Compound | Structure | NMR | SM |
| Example 499 | 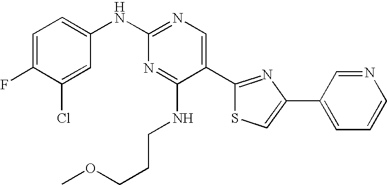 N²-(3-chloro-4-fluorophenyl)-N⁴-(3-methoxypropyl)- 5-[4-(pyridin-3-yl)-1,3-thiazol-2-yl]pyrimidine-2,4- diamine | MS(ES): 471 (M + 1) for C₂₂H₂₀ClFN₆OS. 400 MHz, DMSO-d₆: δ 1.94- 1.95 (m, 2H), 3.22 (s, 3H), 3.50 (t, J = 6.04 Hz, 2H), 3.70 (q, J = 6.60 Hz, 2H), 7.33 (t, J = 9.12 Hz, 1H), 7.52 (q, J = 4.72 Hz, 1H), 7.64-7.68 (m, 1H), 8.22 (s, 1H), 8.26 (dd, J = 2.48, 6.88 Hz, 1H), 8.34 (d, J = 7.88 Hz, 1H), 8.57-8.60 (m, 2H), 9.22 (d, J = 1.76 Hz, 1H), 9.31 (t, J = 5.40 Hz, 1H), 9.86 (br s, 1H). | 3-bromoacetylpyridine hydrobromide and Intermediate 159 |

### Example 500

### N²-(3-chloro-4-fluorophenyl)-N⁴-(3-methoxypropyl)-5-[4-(pyridin-4-yl)-1,3-thiazol-2-yl]pyrimidine-2,4-diamine

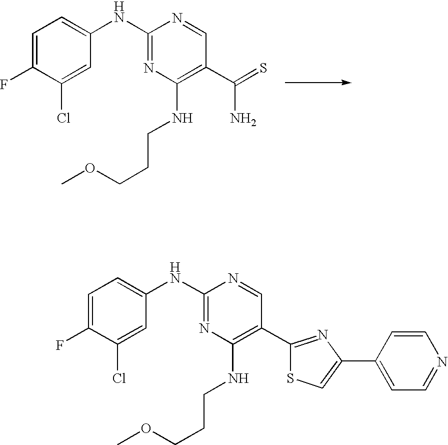

To a suspension of 2-[(3-chloro-4-fluorophenyl)amino]-4-[(3-methoxypropyl)amino]pyrimidine-5-carbothioamide (Intermediate 159), 0.22 mmol, 80 mg) in DMF (2 mL), was added 4-bromoacetylpyridine hydrobromide (0.23 mmol, 65 mg) and warmed to 80° C. for 3 h. After completion of the reaction, as monitored by TLC, the reaction mixture was quenched with water. The solid that precipitated out was filtered. It was further stirred with acetonitrile, filtered and dried to afford the title compound (60 mg).

| | | Mass spectrum and ¹H | |
|---|---|---|---|
| Compound | Structure | NMR | SM |
| Example 500 | 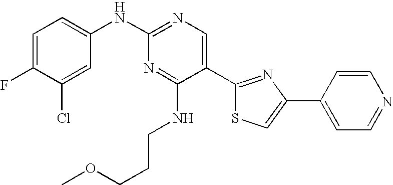 N²-(3-chloro-4-fluorophenyl)-N⁴-(3-methoxypropyl)- 5-[4-(pyridin-4-yl)-1,3-thiazol-2-yl]pyrimidin-2,4-diamine | MS(ES): 471 (M + 1) for C₂₂H₂₀ClFN₆OS. 400 MHz, DMSO-d₆: δ 1.97 (t, J = 6.44 Hz, 2H), 3.23 (s, 3H), 3.50 (t, J = 6.04 Hz, 2H), 3.71 (q, J = 6.48 Hz, 2H), 7.34 (t, J = 9.08 Hz, 1H), 7.64-7.67 (m, 1H), 8.20- 8.21 (m, 2H), 8.25 (dd, J = 2.40, 6.82 Hz, 1H), 8.62 (s, 2H), 8.81-8.82 (m, 2H), 9.22 (br t, 1H), 9.90 (br s, 1H). | 4- bromoacetylpyridine hydrobromide and Intermediate 159 |

### Example 501

### ethyl 5-(2-{2-[(3-chloro-4-fluorophenyl)amino]-4-[(3-methoxypropyl)amino]pyrimidin-5-yl}-1,3-thiazol-4-yl)isoxazole-3-carboxylate

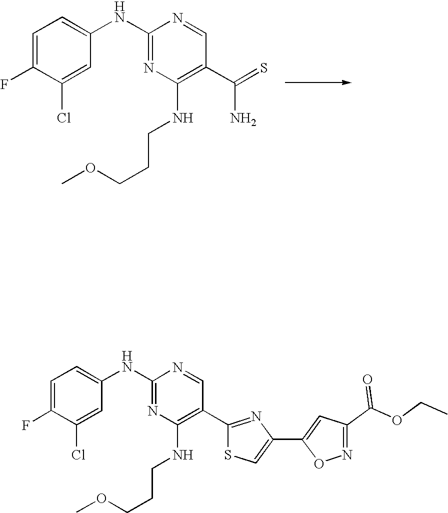

To a suspension of 2-[(3-chloro-4-fluorophenyl)amino]-4-[(3-methoxypropyl)amino]pyrimidine-5-carbothioamide (Intermediate 159, 0.54 mmol, 200 mg) and magnesium sulfate heptahydrate (0.65 mmol, 160 mg) in dry acetone (2 mL) under nitrogen atmosphere, was added ethyl 5-(bromoacetyl)-1,2-oxazole-3-carboxylate (0.59 mmol, 155 mg). The resulting mixture was stirred at reflux temperature for 3 hours, concentrated and purified by silica gel column chromatography (60-120 mesh; product eluted at 1% MeOH/CHCl₃) as eluent to afford the title compound (64 mg).

| | | Mass spectrum and ¹H | |
|---|---|---|---|
| Compound | Structure | NMR | SM |
| Example 501 | 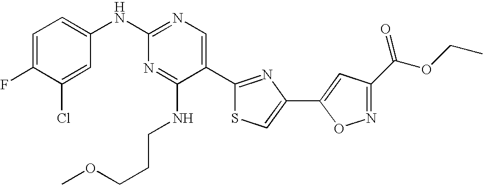 ethyl 5-(2-{2-[(3-chloro-4-fluorophenyl)amino]-4-[(3- methoxypropyl)amino]pyrimidin-5-yl}-1,3-thiazol-4-yl)isoxazole- 3-carboxylate | MS(ES): 533 (M + 1) for C₂₃H₂₂ClFN₆O₄S. 400 MHz, DMSO-d₆: δ 1.35 (t, J = 7.08 Hz, 3H), 1.92-1.95 (m, 2H), 3.22 (s, 3H), 3.51 (t, J = 6.08 Hz, 2H), 3.69 (q, J = 6.48 Hz, 2H), 4.41 (q, J = 7.08 Hz, 2H), 7.33 (t, J = 9.12 Hz, 1H), 7.46 (s, 1H), 7.64-7.68 (m, 1H), 8.23 (dd, J = 2.68, 6.88 Hz, 1H), 8.39 (s, 1H), 8.63 (s, 1H), 9.17 (t, J = 5.28 Hz, 1H), 9.89 (br s, 1H). | ethyl 5- (bromoacetyl)- 1,2-oxazole-3- carboxylate and Intermediate 159 |

### Example 502

### 5-(2,4′-bi-1,3-thiazol-2′-yl)-N²-(3-chloro-4-fluorophenyl)-N⁴-(3-methoxypropyl)pyrimidine-2,4-diamine

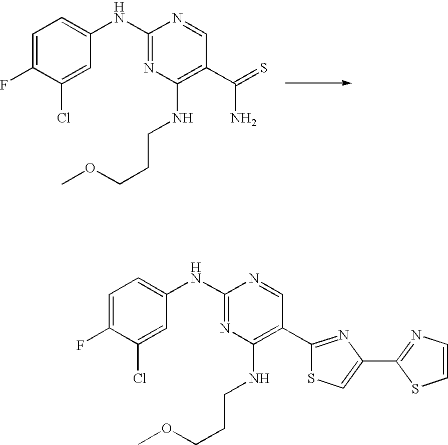

To a suspension of 2-[(3-chloro-4-fluorophenyl)amino]-4-[(3-methoxypropyl)amino]pyrimidine-5-carbothioamide (Intermediate 159), 0.14 mmol, 50 mg) in DMF (2 mL), was added 2-bromo-1-(1,3-thiazol-2-yl)ethanone (0.13 mmol, 27 mg) and warmed to 100° C. for 3 h. The reaction mixture was quenched with water. The solid that precipitated out was filtered, washed with acetonitrile and dried to afford the title compound (35 mg).

| | | Mass spectrum and ¹H | |
|---|---|---|---|
| Compound | Structure | NMR | SM |
| Example 502 | 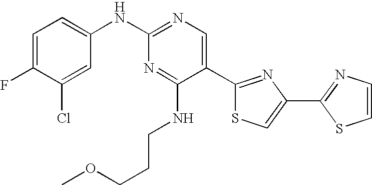 5-(2,4′-bi-1,3-thiazol-2′-yl)-N²-(3-chloro-4- fluorophenyl)-N⁴-(3-methoxypropyl)-pyrimidin- 2,4-diamine | MS(ES): 477 (M + 1) for C₂₀H₁₈ClFN₆OS₂. 400 MHz, DMSO-d₆: δ 1.97- 2.00 (m, 2H), 3.25 (s, 3H), 3.53 (t, J = 6.00 Hz, 2H), 3.67 (q, J = 6.80 Hz, 2H), 7.34 (t, J = 9.20 Hz, 1H), 7.63-7.67 (m, 1H), 7.86 (d, J = 3.20 Hz, 1H), 7.96 (d, J = 3.20 Hz, 1H), 8.18 (s, 1H), 8.28 (dd, J = 2.00, 6.80 Hz, 1H), 8.63 (s, 1H), 9.17 (br t, 1H), 9.92 (br s, 1H). | 2-bromo-1-(1,3- thiazol-2- yl)ethanone and Intermediate 159 |

### Example 503

### N²-(3-chloro-4-fluorophenyl)-N⁴-(3-methoxypropyl)-5-[4-(5-methylisoxazol-4-yl)-1,3-thiazol-2-yl]pyrimidine-2,4-diamine

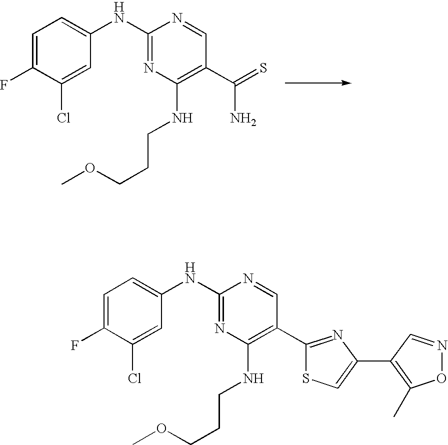

To a suspension of 2-[(3-chloro-4-fluorophenyl)amino]-4-[(3-methoxypropyl)amino]pyrimidine-5-carbothioamide (Intermediate 159, 0.27 mmol, 100 mg) in DMF (2 mL), was added 2-bromo-1-(5-methylisoxazol-4-yl)ethanone (Intermediate 164, 0.2 mmol, 60 mg) and warmed to 80° C. for 3 h. The reaction mixture was quenched with water and the solid that precipitated out was filtered, washed with acetonitrile and dried to afford the title compound (35 mg).

| | | Mass spectrum and ¹H | |
|---|---|---|---|
| Compound | Structure | NMR | SM |
| Example 503 | 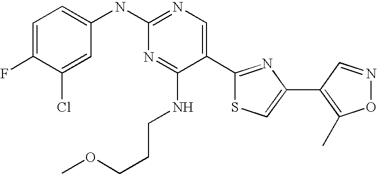 N²-(3-chloro-4-fluorophenyl)-N⁴-(3-methoxypropyl)- 5-[4-(5-methylisoxazol-4-yl)-1,3-thiazol-2- yl]pyrimidine-2,4-diamine | MS(ES): 475 (M + 1) for C₂₁H₂₀ClFN₆O₂S. 400 MHz, DMSO-d₆: δ 1.90- 1.94 (m, 2H), 2.72 (s, 3H), 3.19 (s, 3H), 3.43 (t, J = 6.00 Hz, 2H), 3.64 (q, J = 6.68 Hz, 2H), 7.33 (t, J = 9.12 Hz, 1H), 7.64-7.64 (m, 1H), 7.81 (s, 1H), 8.22 (dd, J = 2.44, 6.84 Hz, 1H), 8.56 (s, 1H), 9.03 (s, 1H), 9.24 (br t, 1H), 9.89 (br s, 1H). | Intermediate 164 2-bromo-1-(5- methylisoxazol- 4-yl)ethanone and Intermediate 159 |

### Example 504

### N²-(3-chloro-4-fluorophenyl)-N⁴-(3-methoxypropyl)-5-[4-(1-methyl-1H-imidazol-5-yl)-1,3-thiazol-2-yl]pyrimidine-2,4-diamine

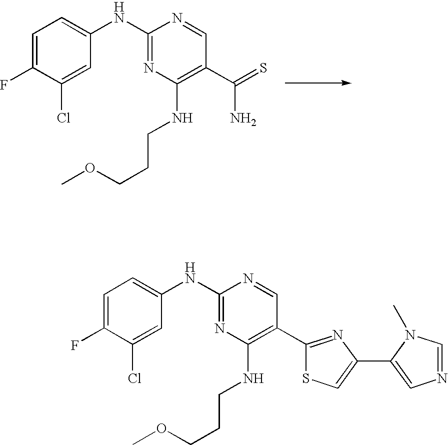

To a suspension of 2-[(3-chloro-4-fluorophenyl)amino]-4-[(3-methoxypropyl)amino]pyrimidine-5-carbothioamide (Intermediate 159, 0.56 mmol, 206 mg,) in ethanol (5 mL), was added the mixture of 2-bromo-1-(1-methyl-1H-imidazol-5-yl)ethanone and 2,2-dibromo-1-(1-methyl-1H-imidazol-5-yl)ethanone (Intermediate 165, 1.12 mmol based on the former, 226 mg,) and warmed to 80° C. for 3 h. The reaction mixture was concentrated and subjected to purification by RP-HPLC (C18 column (19×250 mm, 7 μm); using a binary solvent mixture of 20 mM NH₄OAc (A)/CH₃CN (B) (0-20 min: 10-60% B, 20-35 min: 60% B and 35-45 min: 60-100% B; flow rate of 15 mL/min; Separation was monitored at 210 and 254 nm) to give the title compound (98 mg).

| | | Mass spectrum and ¹H | |
|---|---|---|---|
| Compound | Structure | NMR | SM |
| Example 504 | 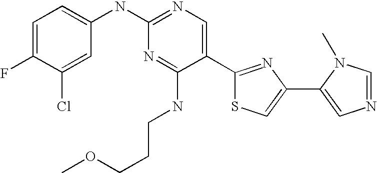 N²-(3-chloro-4-fluorophenyl)-N⁴-(3-methoxypropyl)- 5-[4-(1-methyl-1H-imidazol-5-yl)-1,3-thiazol-2- yl]pyrimidine-2,4-diamine | MS(ES): 474 (M + 1) for C₂₁H₂₁ClFN₇OS. 400 MHz, CH₃COOD: δ 2.10 (m, 2H), 3.29 (s, 3H), 3.62 (t, J = 6.00 Hz, 2H), 3.85 (t, J = 6.80 Hz, 2H), 4.17 (s, 3H), 7.24 (t, J = 8.80 Hz, 1H), 7.59-7.63 (m, 1H), 7.98-8.00 (m, 2H), 8.15 (dd, J = 2.80, 6.60 Hz, 1H), 8.73 (s, 1H), 8.97 (br s, 1H). | Intermediate 165 2-bromo-1-(1- methyl-1H- imidazol-5- yl)ethanone and 2,2-dibromo-1- (1-methyl-1H- imidazol-5- yl)ethanone and Intermediate 159 |

### Example 505

### methyl 2-{2-[(3-chloro-4-fluorophenyl)amino]-4-[(3-methoxypropyl)amino]pyrimidin-5-yl}-4-methyl-1,3-thiazole-5-carboxylate

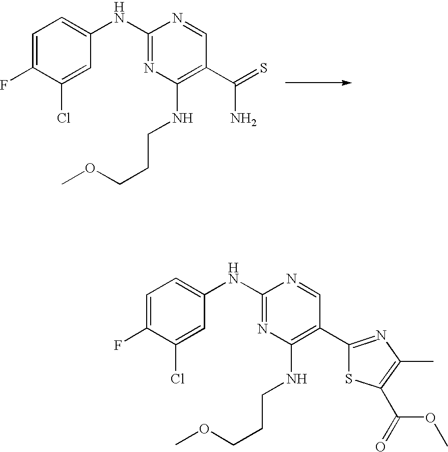

To a solution of 2-[(3-chloro-4-fluorophenyl)amino]-4-[(3-methoxypropyl)amino]pyrimidine-5-carbothioamide (Intermediate 159, 200 mg, 0.5 mmol) in ethanol (1 mL) was added methyl-2-chloroacetoacetate (0.072 mL, 0.089 g, 0.6 mmol). The resulting mixture was subjected to microwave irradiation at 150° C. for 2 h. The precipitated solid was filtered, washed with water and dried to afford the crude product. It was further purified by silica gel column chromatography (60-120 mesh; product eluted at 20% ethyl acetate/hexanes) as eluent to afford the title compound (40 mg).

| | | Mass spectrum and ¹H | |
|---|---|---|---|
| Compound | Structure | NMR | SM |
| Example 505 | 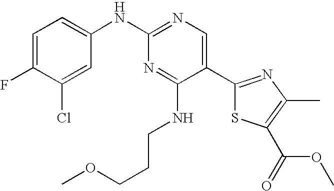 methyl 2-{2-[(3-chloro- 4-fluorophenyl)amino]- 4-[(3- methoxypropyl)amino] pyrimidin-5-yl}-4- methyl-1,3-thiazole-5- carboxylate | MS(ES): 466 (M + 1) for C₂₀H₂₁ClFN₅O₃S. 400 MHz, DMSO-d₆: δ 1.88 (t, J = 6.40 Hz, 2H), 2.66 (s, 3H), 3.25 (s, 3H), 3.46 (t, J = 6.08 Hz, 2H), 3.61 (q, J = 6.80 Hz, 2H), 3.81 (s, 3H), 7.32 (t, J = 9.12 Hz, 1H), 7.65 (ddd, J = 2.72, 4.18, 9.06 Hz, 1H), 8.20 (dd, J = 2.48, 6.78 Hz, 1H), 8.58 (s, 1H), 9.33 (br s, 1H), 9.91 (br s, 1H). | methyl-2- chloroacetoacetate and Intermediate 159 |

### Example 506

### ethyl 2-{2-[(3-chloro-4-fluorophenyl)amino]-4-[(3-methoxypropyl)amino]pyrimidin-5-yl}-4-phenyl-1,3-thiazole-5-carboxylate

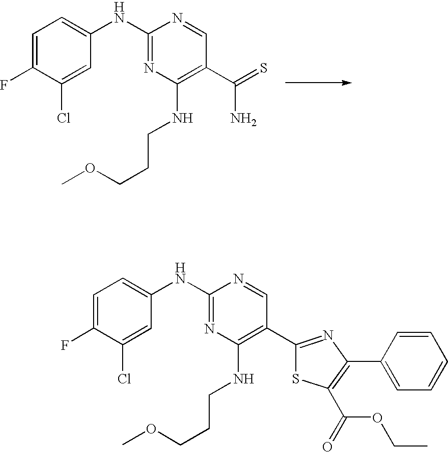

To a solution of 2-[(3-chloro-4-fluorophenyl)amino]-4-[(3-methoxypropyl)amino]pyrimidine-5-carbothioamide (Intermediate 159, 100 mg, 0.27 mmol) in ethanol (10 mL) was added ethyl 2-bromo-3-oxo-3-phenylpropanoate (80 mg, 0.29 mmol). The resulting mixture was stirred overnight at RT. The solvent was removed in vacuo and the slurry taken in ethyl acetate was washed with water and brine. It was dried over sodium sulfate and further purified by silica gel column chromatography (60-120 mesh) using 1% MeOH/CHCl₃ as eluent to afford the title compound (32 mg).

| | | Mass spectrum and ¹H | |
|---|---|---|---|
| Compound | Structure | NMR | SM |
| Example 506 | 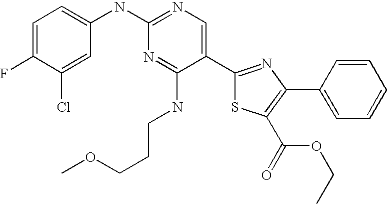 ethyl 2-{2-[(3-chloro- 4-fluorophenyl)amino]- 4-[(3- methoxypropyl)amino] pyrimidin-5-yl}-4- phenyl-1,3-thiazole-5- carboxylate | MS(ES): 542 (M + 1) for C₂₆H₂₅ClFN₅O₃S. 400 MHz, DMSO-d₆: δ 1.21 (t, J = 7.00 Hz, 3H), 1.83 (q, J = 6.12 Hz, 2H), 3.10 (s, 3H), 3.39 (t, J = 6.00 Hz, 2H), 3.59 (q, J = 6.32 Hz, 2H), 4.22 (q, J = 7.20 Hz, 2H), 7.33 (t, J = 9.16 Hz, 1H), 7.47-7.48 (m, 3H), 7.63-7.65 (m, 1H), 7.77-7.78 (m, 2H), 8.22 (d, J = 4.52 Hz, 1H), 8.66 (s, 1H), 9.30 (br t, 1H), 9.98 (br s, 1H). | ethyl 2-bromo- 3-oxo-3- phenylpropanoate and Intermediate 159 |

### Example 507

### ethyl 2-{2-[(3-chloro-4-fluorophenyl)amino]-4-[(3-methoxypropyl)amino]pyrimidin-5-yl}-4-(pyridin-2-yl)-1,3-thiazole-5-carboxylate

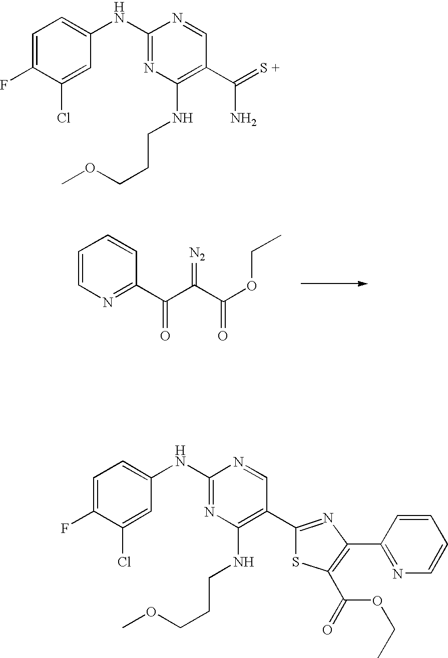

To 2-[(3-chloro-4-fluorophenyl)amino]-4-[(3-methoxypropyl)amino]pyrimidine-5-carbothioamide (Intermediate 159, 1 mmol, 0.37 g) and [1-ethoxy-1,3-dioxo-3-(pyridin-2-yl)propan-2-ylidene]diazenium (Intermediate 168, 0.91 mmol, 200 mg) taken in dry toluene (5 mL), Copper(I) bromide dimethyl sulfide complex (0.76 mmol, 157 mg) was added and heated at 110° C. for 0.5 h. The reaction mixture was cooled to room temperature, quenched with water and extracted with ethyl acetate. The organic layer was washed with brine (50 mL×2) and dried over sodium sulphate. The solvent was removed under vacuum to afford a solid, which was purified by silica gel column chromatography using chloroform:methanol (9:1) as eluent. It was further purified by RP-HPLC (Kromasil C18 column (50×250 mm, 10 μm); using a binary solvent mixture of 20 mM NH₄OAc (A)/CH₃CN (B) (0-20 min: 10-80% B, 20-30 min: 80% B, flow rate of 40 mL/min; Separation was monitored at 210 nm and 254 nm) to give the title compound (42 mg).

| | | Mass spectrum and | |
|---|---|---|---|
| Compound | Structure | ¹H NMR | SM |
| Example 507 | 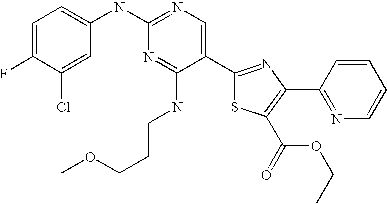 ethyl 2-{2-[(3-chloro-4- fluorophenyl)amino]-4- [(3- methoxypropyl)amino]- pyrimidin-5-yl}-4-(pyridin- 2-yl)-1,3-thiazole-5- carboxylate | MS(ES): 543 (M + 1) for C₂₅H₂₄ClFN₆O₃S. 400 MHz, DMSO-d₆: δ 1.15 (t, J = 7.04 Hz, 3H), 1.82-1.84 (m, 2H), 3.10 (s, 3H), 3.40 (t, J = 6.12 Hz, 2H), 3.60 (q, J = 6.52 Hz, 2H), 4.20 (q, J = 7.16 Hz, 2H), 7.34 (t, J = 9.08 Hz, 1H), 7.46-7.49 (m, 1H), 7.63- 7.64 (m, 1H), 7.85 (d, J = 7.80 Hz, 1H), 7.93 (dd, J = 1.72, 7.64 Hz, 1H), 8.22 (dd, J = 2.48, 6.80 Hz, 1H), 8.66 (s, 1H), 8.67 (s, 1H), 9.27 (br t, 1H), 9.98 (br s, 1H). | Intermediate 168 [1-ethoxy-1,3- dioxo-3- (pyridin-2- yl)propan-2- ylidene] diazenium and Intermediate 159 |

### Example 508

### ethyl 2-{2-[(3-chloro-4-fluorophenyl)amino]-4-[(3-methoxypropyl)amino]pyrimidin-5-yl}-4-(1-methyl-1H-pyrazol-3-yl)-1,3-thiazole-5-carboxylate

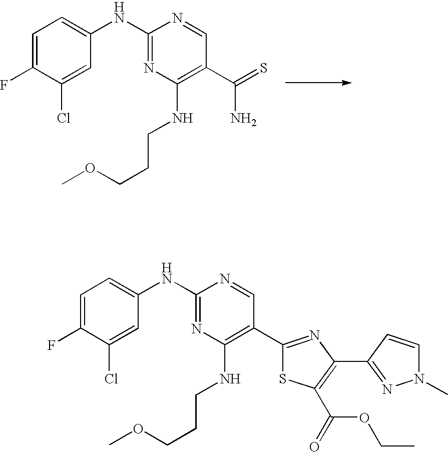

To a solution of 2-[(3-chloro-4-fluorophenyl)amino]-4-[(3-methoxypropyl)amino]pyrimidine-5-carbothioamide (Intermediate 159, 95 mg, 0.25 mmol) in ethanol (10 mL) was added ethyl 2-chloro-3-(1-methyl-1H-pyrazol-3-yl)-3-oxopropanoate (Intermediate 171, 130 mg, 0.56 mmol). The resulting mixture was refluxed at 90° C. for 3 d. The solvent was removed in vacuo and the slurry was taken in ethyl acetate and washed with water and brine. The organic solution was dried over sodium sulfate and further purified by silica gel column chromatography (60-120 mesh) using 1% MeOH/CHCl₃ as eluent to afford the title compound (24 mg).

| | | Mass spectrum and¹H | |
|---|---|---|---|
| Compound | Structure | NMR | SM |
| Example 508 | 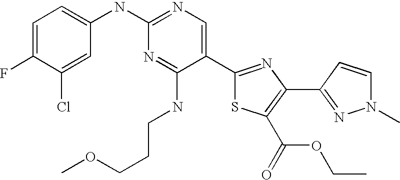 ethyl 2-{2-[(3-chloro- 4-fluorophenyl)amino]- 4-[(3- methoxypropyl)amino] pyrimidin-5-yl}-4-(1- methyl-1H-pyrazol-3- yl)-1,3-thiazole-5- carboxylate | MS(ES): 546 (M + 1) for C₂₄H₂₅ClFN₇O₃S. 400 MHz, DMSO-d₆: δ 1.29 (t, J = 7.20 Hz, 3H), 1.94 (m, J = 6.40 Hz, 2H), 3.21 (s, 3H), 3.53 (t, J = 6.00 Hz, 2H), 3.62 (q, J = 6.40 Hz, 2H), 3.94 (s, 3H), 4.29 (q, J = 6.80 Hz, 2H), 7.03 (d, J = 2.00 Hz,1H), 7.35 (t, J = 8.80 Hz, 1H), 7.64-7.67 (m, 1H), 7.81 (d, J = 2.00 Hz, 1H), 8.25 (dd, J = 2.00, 6.40 Hz, 1H), 8.66 (s, 1H), 9.72 (br t, J = 4.80 Hz, 1H), 9.96 (br s, 1H). | Intermediate 171 ethyl 2-chloro- 3-(1-methyl- 1H-pyrazol-3- yl)-3- oxopropanoate and Intermediate 159 |

### Example 509

### 2-{2-[(3-chloro-4-fluorophenyl)amino]-4-[(3-methoxypropyl)amino]pyrimidin-5-yl}-4-methyl-1,3-thiazole-5-carboxylic acid

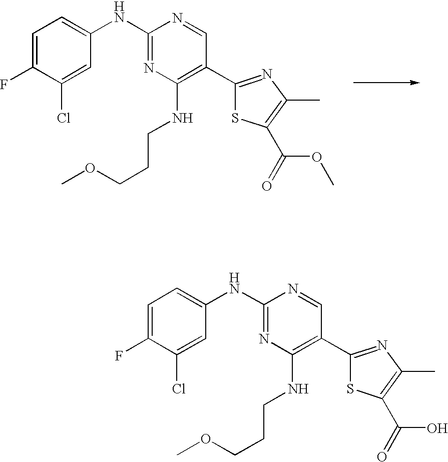

Example 505 (0.16 mmol 75 mg,) was dissolved in tetrahydrofuran (2 mL) and treated with 1 N aq. NaOH (0.6 mL). The reaction mixture was warmed to 60° C. for 24 h. The reaction mixture was concentrated and the aqueous layer carefully acidified with 1.5 N HCl. The solid that precipitated was filtered out, washed with water and dried under vacuum to yield the title compound (15 mg).

| | | Mass spectrum and ¹H | |
|---|---|---|---|
| Compound | Structure | NMR | SM |
| Example 509 | 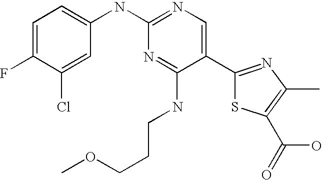 2-{2-[(3-chloro-4- fluorophenyl)amino]-4- [(3- methoxypropyl)amino] pyrimidin-5-yl}-4- methyl-1,3-thiazole-5- carboxylic acid | MS(ES): 452 (M + 1) for C₁₉H₁₉ClFN₅O₃S. 400 MHz, DMSO-d₆: δ 1.89 (t, J = 6.00 Hz, 2H), 2.66 (s, 3H), 3.27 (s, 3H), 3.47 (t, J = 5.60 Hz, 2H), 3.63 (d, J = 5.60 Hz, 2H), 7.34 (t, J = 8.80 Hz, 1H), 7.65 (d, J = 8.40 Hz, 1H), 8.22 (d, J = 6.40 Hz, 1H), 8.58 (s, 1H), 9.38 (br s, 1H), 9.91 (br s, 1H), 13.29 (br s, 1H). | Example 505 methyl 2-{2- [(3-chloro-4- fluorophenyl) amino]-4-[(3- methoxypropyl) amino] pyrimidin- 5-yl}-4- methyl-1,3- thiazole-5- carboxylate |

### Example 510

### 2-{2-[(3-chloro-4-fluorophenyl)amino]-4-[(3-methoxypropyl)amino]pyrimidin-5-yl}-4-phenyl-1,3-thiazole-5-carboxylic acid

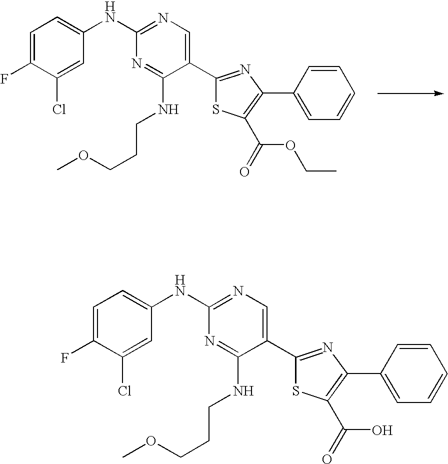

To a suspension of 150 mg of Example 506 (0.28 mmol, 150 mg) taken in tetrahydrofuran (2.5 mL) and water (2.5 mL), Lithium hydroxide monohydrate (1.11 mmol, 46 mg) was added and the reaction was warmed overnight at 60° C. After completion of reaction, the reaction mixture was concentrated and the aqueous layer was carefully acidified with 1.5 N HCl. The solid that precipitated was filtered out, washed with water and dried under vacuum to yield the title compound (110 mg).

| | | Mass spectrum and ¹H | |
|---|---|---|---|
| Compound | Structure | NMR | SM |
| Example 510 | 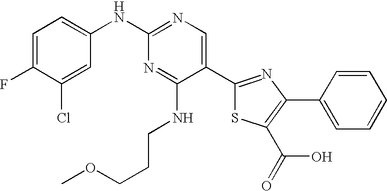 2-{2-[(3-chloro-4- fluorophenyl)amino]-4- [(3- methoxypropyl)amino] pyrimidin-5-yl}-4- phenyl-1,3-thiazole-5- carboxylic acid | MS(ES): 514 (M + 1) for C₂₄H₂₁ClFN₅O₃S. 400 MHz, DMSO-d₆: δ 1.84 (t, J = 6.28 Hz, 2H), 3.11 (s, 3H), 3.40 (t, J = 6.00 Hz, 2H), 3.61 (t, J = 6.04 Hz, 2H), 7.34 (t, J = 9.12 Hz, 1H), 7.46-7.47 (m, 3H), 7.64-7.66 (m, 1H), 7.78-7.79 (m, 2H), 8.23 (d, J = 5.00 Hz, 1H), 8.65 (br s, 1H), 9.33 (br t, 1H), 9.95 (br s, 1H), 13.50 (br s, 1H). | Example 506 ethyl 2-{2-[(3- chloro-4- fluorophenyl) amino]-4-[(3- methoxypropyl) amino] pyrimidin- 5-yl}-4- phenyl-1,3- thiazole-5- carboxylate |

### Example 511

### 2-{2-[(3-chloro-4-fluorophenyl)amino]-4-[(3-methoxypropyl)amino]pyrimidin-5-yl}-4-(pyridin-2-yl)-1,3-thiazole-5-carboxylic acid

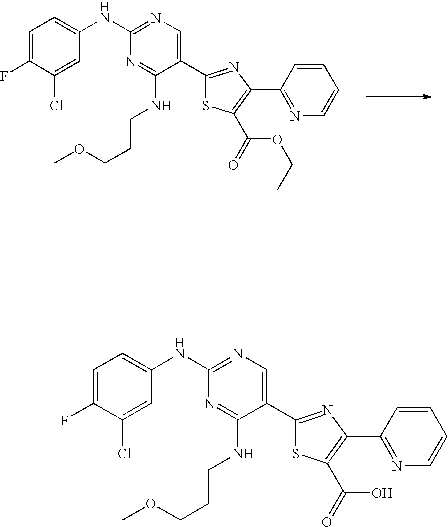

To 40 mg of ethyl 2-{2-[(3-chloro-4-fluorophenyl)amino]-4-[(3-methoxypropyl)amino]pyrimidin-5-yl}-4-(pyridin-2-yl)-1,3-thiazole-5-carboxylate (Example 507, 0.07 mmol) taken in THF (1 mL) and water (1 mL), was added Lithium hydroxide monohydrate (0.29 mmol, 12 mg) and the reaction was warmed overnight at 65° C. The reaction mixture was concentrated then the aqueous layer was carefully acidified with 1.5 N HCl. The solid that precipitated was filtered out, washed with water and dried under vacuum to yield 18 mg of the title compound as a yellow powder.

| | | Mass spectrum and ¹H | |
|---|---|---|---|
| Compound | Structure | NMR | SM |
| Example 511 | 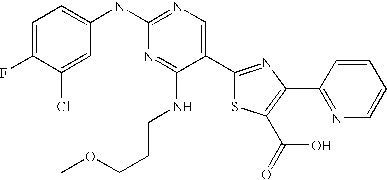 2-{2-[(3-chloro-4- fluorophenyl)amino]-4- [(3- methoxypropyl)amino] pyrimidin-5-yl}-4- (pyridin-2-yl)-1,3- thiazole-5-carboylic acid | MS(ES): 515 (M + 1) for C₂₃H₂₀ClFN₆O₃S. 400 MHz, DMSO-d₆: δ 1.98 (q, J = 6.40 Hz, 2H), 3.23 (s, 3H), 3.51 (t, J = 6.00 Hz, 2H), 3.71 (q, J = 5.60 Hz, 2H), 7.33 (t, J = 8.80 Hz, 1H), 7.64-7.66 (m, 1H), 7.80 (t, J = 6.40 Hz, 1H), 8.23 (d, J = 4.40 Hz, 1H), 8.38 (t, J = 7.20 Hz, 1H), 8.47 (d, J = 8.00 Hz, 1H), 8.66 (s, 1H), 8.85 (d, J = 5.20 Hz, 1H), 9.04 (br s, 1H), 9.98 (br s, 1H). | Example 507 ethyl 2-{2-[(3- chloro-4- fluorophenyl) amino]-4-[(3- methoxypropyl) amino] pyrimidin- 5-yl}-4- (pyridin-2-yl)- 1,3-thiazole-5- carboxylate |

### Example 512

### 2-{2-[(3-chloro-4-fluorophenyl)amino]-4-[(3-methoxypropyl)amino]pyrimidin-5-yl}-4-(1-methyl-1H-pyrazol-3-yl)-1,3-thiazole-5-carboxylic acid

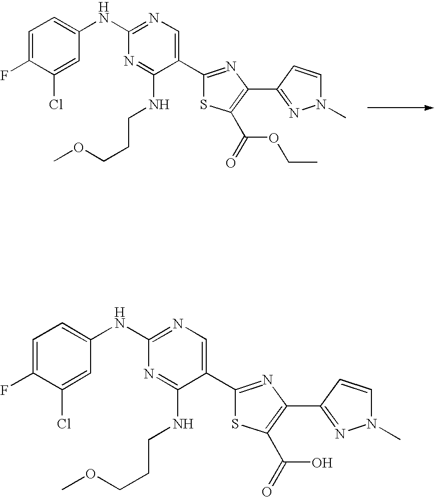

To 80 mg of ethyl 2-{2-[(3-chloro-4-fluorophenyl)amino]-4-[(3-methoxypropyl)amino]pyrimidin-5-yl}-4-(1-methyl-1H-pyrazol-3-yl)-1,3-thiazole-5-carboxylate (Example 508, 0.16 mmol) taken in THF (5 mL) was added Barium hydroxide monohydrate (0.44 mmol, 0.083 g) and water (5 mL). The reaction mixture was allowed to stir at RT overnight. After completion of the reaction, the mixture was then carefully acidified with 1.5 N HCl to pH=2 and the precipitate formed was filtered, washed with water and dried to yield the title compound (24 mg).

| | | Mass spectrum and ¹H | |
|---|---|---|---|
| Compound | Structure | NMR | SM |
| Example 512 | 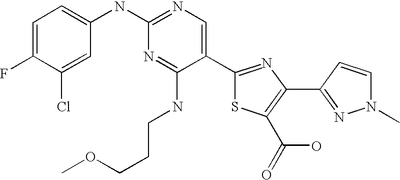 2-{2-[(3-chloro-4- fluorophenyl)amino]-4- [(3- methoxypropyl)amino] pyrimidin-5-yl}-4-(1- methyl-1H-pyrazol-3- yl)-1,3-thiazole-5- carboxylic acid | MS(ES): 518 (M + 1) for C₂₂H₂₁ClFN₇O₃S. 400 MHz, DMSO-d₆: δ 1.95 (q, J = 6.40 Hz, 2H), 3.12 (s, 3H), 3.55 (t, J = 6.08 Hz, 2H), 3.60 (t, J = 5.52 Hz, 2H), 3.88 (s, 3H), 7.31 (t, J = 9.16 Hz, 1H), 7.44-7.45 (m, 1H), 7.63- 7.65 (m, 2H), 8.25 (d, J = 6.68 Hz, 1H), 8.46 (s, 1H), 9.77 (s, 1H), 10.05 (br s, 1H). | Example 508 ethyl 2-{2-[(3- chloro-4- fluorophenyl) amino]-4-[(3- methoxypropyl) amino] pyrimidin- 5-yl}-4-(1- methyl-1H- pyrazol-3-yl)- 1,3-thiazole-5- carboxylate |

### Example 513

### ethyl (2E)-3-{3-[2-{[4-fluoro-3-(hydroxymethyl)phenyl]amino}-4-(morpholin-4-yl)pyrimidin-5-yl]phenyl}prop-2-enoate

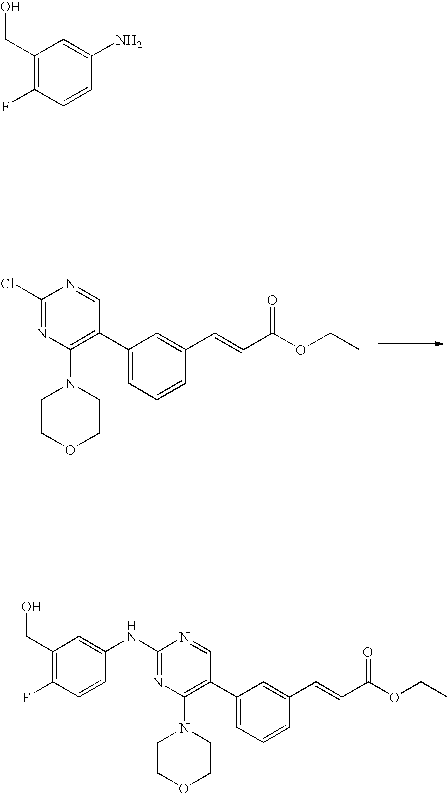

A suspension of (2E)-3-{3-[2-chloro-4-(morpholin-4-yl)pyrimidin-5-yl]phenyl}prop-2-enoate (Intermediate 145) (0.8 mmol, 0.30 g, 1 eq), (5-amino-2-fluorophenyl)methanol (0.88 mmol, 0.12 g, 1.1 eq), tris(dibenzyledeneacetone)dipalladium(0) (0.24 mmol, 0.22 g, 30 mol %), 2-dicyclohexylphosphino-2′,4′,6′-triisopropyl-1,1′-biphenyl (0.24 mmol, 0.11 g, 30 mol %) and sodium carbonate (0.8 mmol, 0.08 g, 1 eq) in acetonitrile/water (6 mL:2 mL) was heated to 90° C. for 30 minutes. The reaction mixture was concentrated and the residue was taken in ethyl acetate (50 mL), washed with water (2×) and brine (1×). The organic layer was separated, dried over sodium sulfate, filtered and concentrated. The crude compound was purified by silica gel column (60-120 mesh) chromatography using hexane:ethyl acetate (3:1) as an eluent to yield the title compound (0.190 g).

| | | Mass spectrum and | |
|---|---|---|---|
| Compound | Structure | ¹H NMR | SM |
| Example 513 | 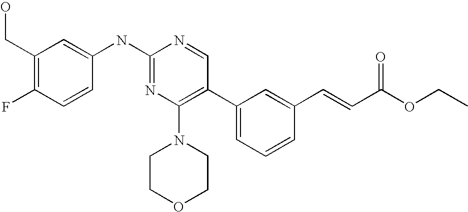 ethyl (2E)-3-{3-[2-{[4-fluoro-3- (hydroxymethyl)phenyl]amino}- 4-(morpholin-4-yl)pyrimidin-5- yl]phenyl}prop-2-enoate | MS(ES): 479 (M + 1) for C₂₆H₂₇FN₄O₄. 300 MHz, DMSO-d₆: δ 1.25 (t, J = 7.14 Hz, 3H), 3.20 (m, 4H), 3.53 (m, 4H), 4.19 (q, J = 7.11 Hz, 2H), 4.57 (br s, 2H), 5.68 (br s, 1H), 6.72 (d, J = 16.11 Hz, 1H), 6.73-6.8 (m, 1H), 7.27 (t, J = 8.31 Hz, 1H), 7.45-7.54 (m, 2H), 7.69 (d, J = 16.29 Hz, 1H), 7.68 (br s, 1H), 7.81 (br s, 1H), 8.02 (d, J = 2.52 Hz, 1H), 8.06 (s, 1H), 8.90 (br s, 1H). | (5-amino- 2-fluoro- phenyl) methanol and Intermediate 145 |

### Example 514

### ethyl (2E)-3-{3-[2-(1H-indol-7-ylamino)-4-(morpholin-4-yl)pyrimidin-5-yl]phenyl}prop-2-enoate

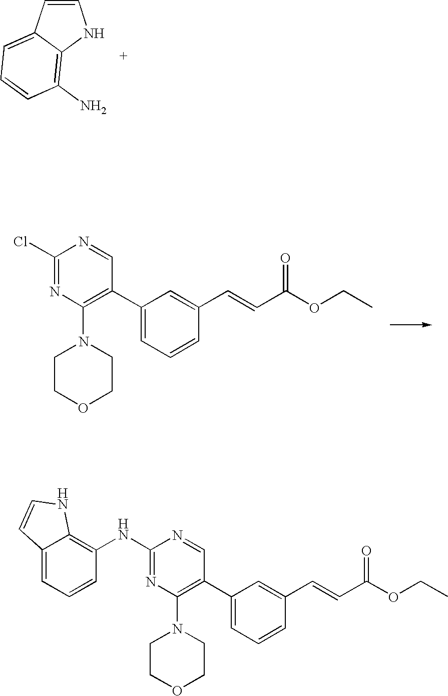

A suspension of (2E)-3-{3-[2-chloro-4-(morpholin-4-yl)pyrimidin-5-yl]phenyl}prop-2-enoate (Intermediate 145) (0.67 mmol, 0.25 g, 1 eq), 1H-indol-7-amine (0.8 mmol, 0.10 g, 1.2 eq), tris(dibenzyledeneacetone)dipalladium(0) (0.2 mmol, 0.184 g, 30 mol %), 2-dicyclohexylphosphino-2′,4′,6′-triisopropyl-1,1′-biphenyl (0.20 mmol, 0.1 g, 30 mol %) and sodium carbonate (0.7 mmol, 0.07 g, 1 eq) in acetonitrile/water (10 mL:2.5 mL) was heated to 90° C. for 30 minutes. The reaction mixture was concentrated and the residue was taken in ethyl acetate (50 mL), washed with water (2×) and brine (1×). The organic layer was separated, dried over sodium sulfate, filtered and concentrated. The crude compound was purified by silica gel column (230-400 mesh) chromatography using chloroform:methanol (98:2) as an eluent to yield the title compound (0.17 g).

| | | Mass spectrum and ¹H | |
|---|---|---|---|
| Compound | Structure | NMR | SM |
| Example 514 | 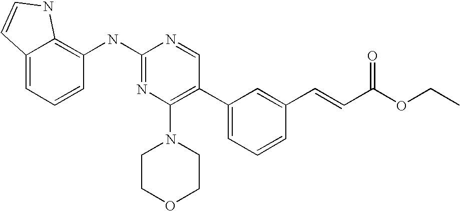 ethyl (2E)-3-{3-[2-(1H-indol- 7-ylamino)-4-(morpholin-4- yl)pyrimidin-5- yl]phenyl}prop-2-enoate | MS(ES): 470 (M + 1) for C₂₇H₂₇N₅O₃. 400 MHz, DMSO-d₆: δ 1.26 (t, J = 7.08 Hz, 3H), 3.20 (br s, 4H), 3.52-3.53 (m, 4H), 4.20 (q, J = 7.04 Hz, 2H), 6.42 (br s, 1H), 6.72 (d, J = 16..04 Hz, 1H), 6.95 (t, J = 7.76 Hz, 1H), 7.22 (d, J = 7.80 Hz, 1H), 7.31 (t, J = 2.60 Hz, 1H), 7.48 (t, J = 7.60 Hz, 1H), 7.54 (d, J = 7.64 Hz, 1H), 7.66 (d, J = 7.88 Hz, 1H), 7.70 (d, J = 16.12 Hz, 1H), 7.77 (d, J = 7.60 Hz, 1H), 7.82 (br s, 1H), 8.07 (s, 1H), 9.05 (br s, 1H), 11.00 (br s, 1H). | 1H-indol-7- amine and Intermediate 145 |

### General Procedure for Hydrolysis of Carboxylic Acid Ester to Acid

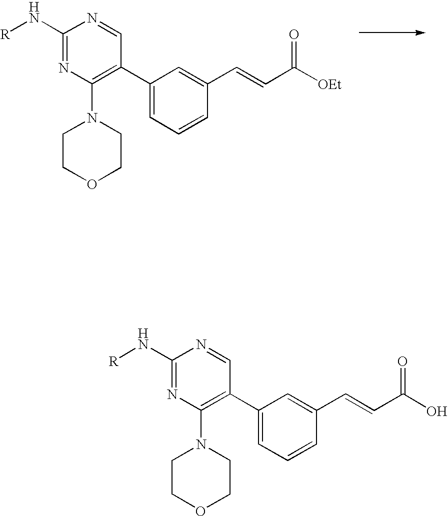

To ester derivative (1 eq) suspended in tetrahydrofuran (1 mL) was added 1 N aq. NaOH (4 eq) and stirred overnight at room temperature. After completion of reaction, the reaction mixture was carefully acidified with 1 N HCl and the solid that precipitated was filtered, washed with water and dried in vacuo to yield the desired carboxylic acid product.

The compounds in the below table were prepared using this method and the indicated starting material.

| | | Mass spectrum and | |
|---|---|---|---|
| Compound | Structure | ¹H NMR | SM |
| Example 516 | 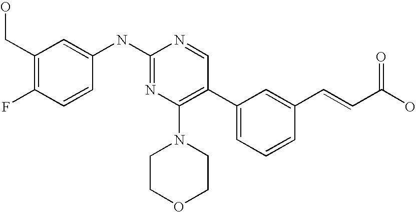 (2E)-3-{3-[2-{[4-fluoro- 3- (hydroxymethyl)phenyl] amino}-4-(morpholin-4- yl)pyrimidin-5- yl]phenyl}prop-2-enoic acid | MS(ES): 451 (M + 1) for C₂₄H₂₃FN₄O₄ 400 MHz, DMSO-d₆: δ 3.22 (br s, 4H), 3.54 (br s, 4H), 4.59 (d, J = 3.60 Hz, 2H), 5.68 (br s, 1H), 6.61 (d, J = 16.00 Hz, 1H), 6.75- 6.80 (m, 1H), 7.28 (t, J = 7.20 Hz, 1H), 7.47- 7.53 (m, 2H), 7.62- 7.66 (m, 2H), 7.79 (s, 1H), 8.05-8.08 (m, 2H), 8.90 (s, 1H), 12.44 (br s, 1H). | Example 513 ethyl (2E)-3-{3-[2- {[4-fluoro-3- hydroxymethyl) phenyl]amino-4- (morpholin-4- yl)pyrimidin-5- yl]phenyl{prop-2- enoate |
| Example 517 | 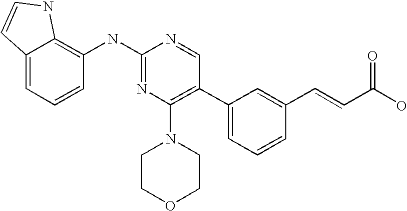 (2E)-3-{3-[2-(1H-indol- 7-ylamino)-4- (morpholin-4-yl) pyrimidin-5-yl] phenyl}prop-2-enoic acid | MS(ES): 442 (M + 1) for C₂₅H₂₃N₅O₃. 400 MHz, DMSO-d₆: δ 3.20 (br s, 4H), 3.53 (br s, 4H), 6.42 (br s, 1H), 6.61 (d, J = 16.08 Hz, 1H), 6.95 (t,J = 7.84 Hz, 1H), 7.22 (d, J = 7.72 Hz, 1H), 7.31 (br s, 1H), 7.47 (t, J = 7.52 Hz, 1H), 7.49-7.54(m, 1H), 7.62 (br s 1H), 7.65 (d, J = 8.08 Hz, 1H), 7.77 (d, J = 8.48 Hz, 1H), 7.78 (br s, 1H), 8.06 (br s, 1H), 9.05 (br s, 1H), 11.00 (br s, 1H), 12.42 (br s, 1H). | Example 514 ethyl (2E)-3-{3-[2- (1H-indol-7-ylamino)-4- (morpholin-4-yl) pyrimidin-5-yl]phenyl} prop-2-enoate |

### General methods for the synthesis of ethyl 5-{2-[(3-chloro-4-fluorophenyl)amino]-4-(azol-1-yl)pyrimidin-5-yl}pyridine-3-carboxylate

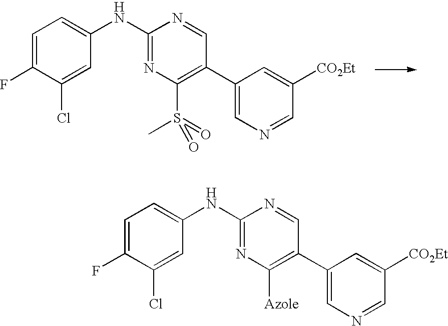

### Method A:

A solution of azole (2.2 eq) in DMF (1 mL) was added slowly to a suspension of sodium hydride (2.1 eq) in DMF (1 mL). The reaction mixture was stirred for 30 min at room temperature. A solution of ethyl 5-(2-(3-chloro-4-fluorophenylamino)-4-(methylsulfonyl)pyrimidin-5-yl)nicotinate Intermediate 124 (1 eq) in DMF (1 mL) was added slowly to the reaction mixture at 0° C. with stiffing and allowed to warm to ambient temperature over 2.5 h. Water was added (˜6 mL) and the solid formed was filtered and dried to yield the product.

### Method B:

A solution of azole (2.2 eq) in DMSO (1 mL) was added slowly to a suspension of sodium hydride (2.1 eq) in DMSO (1 mL). The reaction mixture was stirred for 30 min at room temperature. A solution of Intermediate 124 (1 eq) in DMSO (1 mL) was added slowly to the reaction mixture at 0° C. with stirring and allowed to warm to ambient temperature over 2.5 h. Water was added (˜6 mL) and the solid formed was filtered and dried to yield the product.

The compounds in the below table were prepared using the methods described above as indicated and the starting material specified.

| Compound | Structure | Mass spectrum and ¹H NMR | SM |
|---|---|---|---|
| Example 519^{a)} | 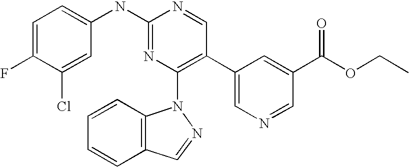 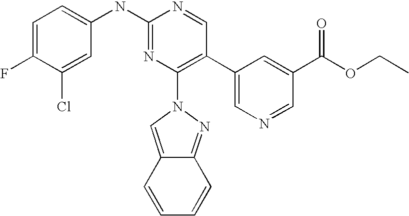 ethyl 5-{2-[(3-chloro-4- fluorophenyl)amino]-4- (1H-indazol-1- yl)pyrimidin-5- yl}pyridine-3- carboxylate & ethyl 5-{2- [(3-chloro-4- fluorophenyl)amino]-4- (2H-indazol-2- yl)pyrimidin-5- yl}pyridine-3- carboxylate | ¹H NMR 400 MHz, DMSO-d₆: δ 1.23 (t, J = 7.15 Hz, 3H), 1.28 (t, J = 7.15 Hz, 3H), 4.24-4.34 (m, 4H), 7.09 (dd, J = 6.90, 8.16 Hz, 1H), 7.20-7.27 (m, 1H), 7.31- 7.47 (m, 3H), 7.59 (t, J = 7.78 Hz, 1H), 7.62-7.70 (m, 1H), 7.74-7.87 (m, 3H), 8.03-8.10 (m, 3H), 8.14-8.17 (m, 1H),8.16- 8.21 (m, 2H), 8.45-8.52 (m, 1H), 8.56 (d, J = 2.26 Hz, 1H), 8.63 (d, J = 2.01 Hz, 1H), 8.71 (s, 1H), 8.86 (s, 1H), 8.95-9.01 (m, 3H), 10.26 (s, 1H), 10.47 (s, 1H). | 1H-indazole |
| Example 520^{c)} | 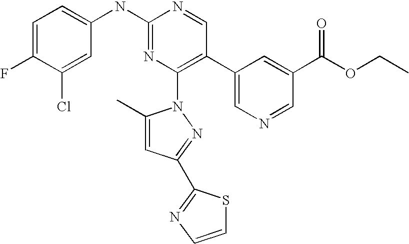 ethyl 5-{2-[(3-chloro-4- fluorophenyl)amino]-4- [5-methyl-3-(1,3-thiazol- 2-yl)-1H-pyrazol-1- yl]pyrimidin-5- yl}pyridine-5- carboxylate | MS(ES): 536 (M + 1) for C₂₅H₁₉ClFN₇O₂S. 300 MHz, DMSO-d₆: δ 1.19 (t, J = 7.08 Hz, 3H), 2.5 (s, 3H, merges with DMSO peak), 4.24 (q, J = 7.11 Hz, 2H), 6.77 (s, 1H), 7.42 (t, J = 9.03 Hz, 1H), 7.65-7.67 (m, 2H), 7.83 (d, J = 3.18 Hz, 1H), 7.95 (s, 1H), 8.09 (d, J = 6.51 Hz, 1H), 8.61 (s, 1H), 8.92 (s, 1H), 8.95 (s, 1H), 10.38 (br s, 1H). | 2-(5- methyl- 1H- pyrazol- 3-yl)-1,3- thiazole |
| Example 521^{c)} | 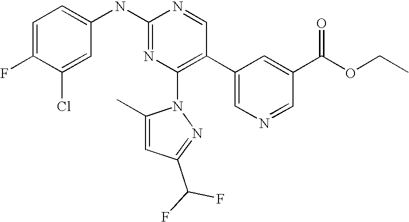 ethyl 5-{2-[(3-chloro-4- fluorophenyl)amino]-4- [3-(difluoromethyl)-5- methyl-1H-pyrazol-1- yl]pyrimidin-5- yl}pyridine-3- carboxylate | MS(ES): 503 (M + 1) for C₂₃H₁₈ClF₃N₆O₂. 300 MHz, DMSO-d₆: δ 1.29 (t, J = 7.05 Hz, 3H), 2.41 (s, 3H), 4.30 (q, J = 7.05 Hz, 2H), 6.56 (s, 1H), 6.73 (t, J = 54.18 Hz, 1H), 7.42 (t, J = 9.15 Hz, 1H), 7.62-7.68 (m, 1H), 7.81 (t, J = 2.04 Hz, 1H), 8.06 (dd, J = 2.4, 6.6 Hz, 1H), 8.55 (d, J = 2.22 Hz, 1H), 8.96 (d, J = 1.92 Hz, 1H), 8.97 (s, 1H), 10.45 (br s, 1H). | 3- (difluoro methyl)- 5-methyl- 1H- pyrazole |
| Example 522^{c)} | 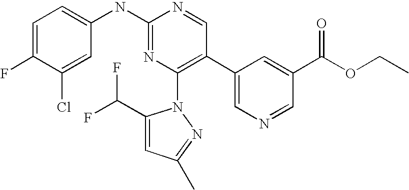 ethyl 5-{2-[(3-chloro-4- fluorophenyl)amino]-4- [5-(difluoromethyl)-3- methyl-1H-pyrazol-1- yl]pyrimidin-5- yl}pyridine-3- carboxylate | MS(ES): 503 (M + 1) for C₂₃H₁₈ClF₃N₆O₂. 400 MHz, DMSO-d₆: δ 1.32 (t, J = 6.80 Hz, 3H), 1.98 (s, 3H), 4.34 (q, J = 7.20 Hz, 2H), 6.76 (s, 1H), 7.42 (t, J = 9.20 Hz, 1H), 7.60-7.64 (m, 1H), 7.96 (t, J = 2.00 Hz, 1H), 8.06 (d, J = 6.80 Hz, 1H), 8.58 (d, J = 2.40 Hz, 1H), 8.86 (s, 1H), 9.00 (d, J = 2.00 Hz, 1H), 10.26 (br s, 1H). | 3- (difluoro methyl)- 5-methyl- 1H-pyrazole |
| Example 523^{b)} | 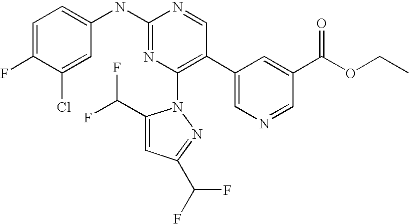 ethyl 5-{4-[3,5- bis(difluoromethyl)-1H- pyrazol-1-yl]-2-[(3- chloro-4- fluorophenyl)amino] pyrimidin-5-yl}pyridine-3- carboxylate | MS(ES): 539 (M + 1) for C₂₃H₁₆ClF₅N₆O₂. 400 MHz, DMSO-d₆: δ 1.29 (t, J = 6.80 Hz, 3H), 4.31 (q, J = 7.20 Hz, 2H), 6.82 (t, J = 53.76 Hz, 1H), 7.25 (s, 1H), 7.40 (t, J = 9.20 Hz, 1H), 7.57 (t, J = 54.80 Hz, 1H), 7.62 (ddd, J = 2.80, 4.00, 9.10 Hz, 1H), 7.86 (t, J = 2.00 Hz, 1H), 8.03 (d, J = 4.40 Hz, 1H), 8.62 (d, J = 2.00 Hz, 1H), 8.97 (s, 1H), 8.99 (d,J = 2.00 Hz, 1H), 10.41 (br s, 1H). | 3,5- bis(di- fluoro- methyl)- 1H- pyrazole |
| Example 524^{d)} | 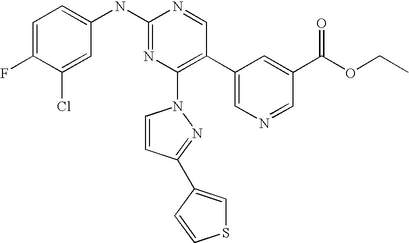 ethyl 5-{2-[(3-chloro-4- fluorophenyl)amino]-4- [3-(thiophen-3-yl)-1H- pyrazol-1-yl]pyrimidin- 5-yl}pyridine-3- carboxylate | MS(ES): 519 (M − 1) for C₂₅H₁₈ClFN₆O₂S. 300 MHz, DMSO-d₆: δ 1.27 (t, J = 7.11 Hz, 3H), 4.32 (q, J = 6.54 Hz, 2H), 6.88 (d, J = 5.04 Hz, 1H), 6.97 (d, 1H), 7.43 (t, J = 9.24 Hz, 1H), 7.52 (m, 1H), 7.66 (br s, 1H), 7.72 (m, 1H), 8.07 (d, J = 4.23 Hz, 1H), 8.24 (br s, 1H), 8.51 (d, 1H), 8.64 (s, 1H), 8.76 (s, 1H), 9.07 (d, 1H), 10.28 (br s, 1H). | 3- (thiophen- 3-yl)- 1H- pyrazole |
| Example 525^{c)} | 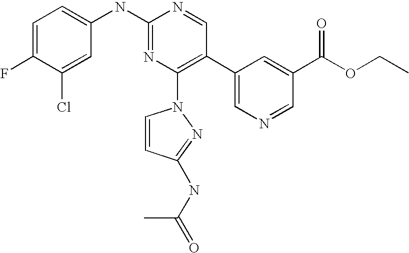 ethyl 5-{4-[3- (acetylamino)-1H- pyrazol-1-yl]-2-[(3- chloro-4- fluorophenyl)amino] pyrimidin-5-yl}pyridine-3- carboxylate | MS(ES): 496 (M + 1) for C₂₃H₁₉ClFN₇O₃. 300 MHz, DMSO-d₆: δ 1.30 (t, J = 7.08 Hz, 3H), 2.53 (s, 3H), 4.34 (q, J = 7.08 Hz, 2H), 6.85 (br d, 1H), 7.41 (t, J = 9.18 Hz, 1H), 7.72 (m, 1H), 8.04 (dd, J = 4.20 Hz, 1H), 8.09 (t, 1H), 8.34 (d, J = 2.61 Hz, 1H), 8.63 (d, J = 1.92 Hz, 1H), 8.65 (s, 1H), 9.01 (d, J = 1.92 Hz, 1H), 10.25 (br s, 1H), 10.28 (s, 1H). | N-(2H- Pyrazol- 3-yl)- acetamide |
| Example 526^{b)} | 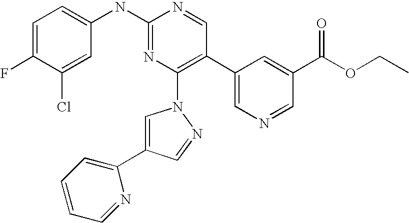 ethyl 5-{2-[(3-chloro-4- fluorophenyl)amino]-4- [4-(pyridin-2-yl)-1H- pyrazol-1-yl]pyrimidin- 5-yl}pyridine-3- carboxylate | MS(ES): 516 (M + 1) for C₂₆H₁₉ClFN₇O₂. 400 MHz, DMSO-d₆: δ 1.30 (t, J = 6.80 Hz, 3H), 4.34 (q, J = 7.08 Hz, 2H), 7.30 (m, 1H), 7.44 (t, J = 9.20 Hz, 1H), 7.70-7.74 (m, 1H), 7.77-7.86 (m, 2H), 8.15 (dd, J = 2.80, 6.80 Hz, 1H), 8.18 (s, 2H), 8.59 (d, J = 4.00 Hz, 1H), 8.72-8.73 (m, 2H), 9.04 (s, 2H), 10.38 (s, 1H). | 2-(1H- pyrazol- 4- yl)pyridine |
| Example 527^{a)} | 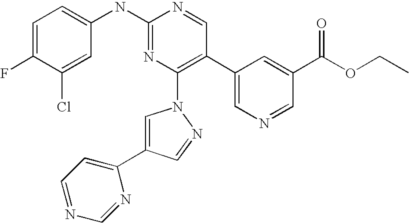 ethyl 5-{2-[(3-chloro-4- fluorophenyl)amino]-4- [4-(pyrimidin-4-yl)-1H- pyrazol-1-yl]pyrimidin- 5-yl}pyridine-3- carboxylate | MS(ES): 515 (M − 1) for C₂₅H₁₈ClFN₈O₂. 400 MHz, DMSO-d₆: δ 1.29 (t, J = 7.08 Hz, 3H), 4.33 (q, J = 7.08 Hz, 2H), 7.44 (t, J = 9.08 Hz, 1H), 7.70-7.74 (m, 1H), 7.88 (dd, J = 1.20, 5.24 Hz, 1H), 8.13 (dd, J = 2.60, 6.70 Hz, 1H), 8.17 (t, J = 2.00 Hz, 1H), 8.30 (s, 1H), 8.71 (d, J = 2.12 Hz, 1H), 8.77 (s, 1H), 8.80 (d, J = 5.32 Hz, 1H), 9.03 (d, J = 1.88 Hz, 1H), 9.15 (d, J = 1.08 Hz, 1H), 9.20 (s, 1H), 10.41 (br s, 1H). | 4-(1H- pyrazol- 4- yl)pyrimidine |

^{a)}Method A
^{b)}Method A, overnight
^{c)}Method B
^{d)}Method B, 125° C., 4 h

### General method for the synthesis of 5-{2-[(3-chloro-4-fluorophenyl)amino]-4-(azol-1-yl)pyrimidin-5-yl}-pyridine-3-carboxylic acid

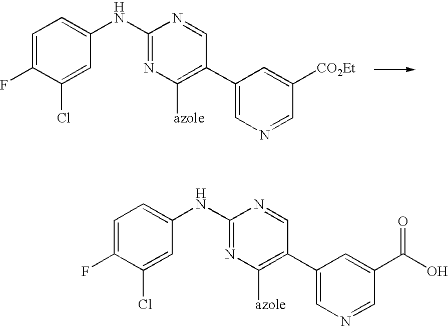

### Method C:

A solution of ethyl 5-{2-[(3-chloro-4-fluorophenyl)amino]-4-(azol-1-yl)pyrimidin-5-yl}pyridine-3-carboxylate derivative (1 eq) in a mixture of tetrahydrofuran (1 mL) and water (1 mL) was treated with 1 N aq. sodium hydroxide (4 eq) and allowed to stir at room temperature for 3-8 h. After completion of the reaction, the mixture was carefully acidified with 1 N HCl and the precipitate formed was filtered, washed with water and dried to yield the product.

### Method D:

A solution of ethyl 5-{2-[(3-chloro-4-fluorophenyl)amino]-4-(azol-1-yl)pyrimidin-5-yl}pyridine-3-carboxylate derivative (1 eq) in a mixture of dioxane (1 mL) and water (1 mL) was treated with 1 N aq. Barium hydroxide (2 eq) and allowed to stir at room temperature for 2-8 h. After completion of the reaction, the mixture was carefully acidified with 1 N HCl and the precipitate formed was filtered, washed with water, dried to yield the product.

The compounds in the below table were prepared using the methods described above as indicated and the starting material specified.

| | | Mass spectrum and | |
|---|---|---|---|
| Compound | Structure | ¹H NMR | SM |
| Example 528^{b)} | 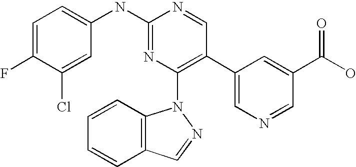 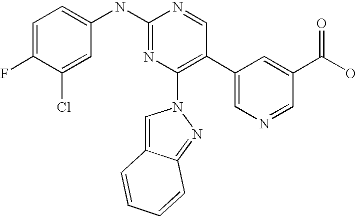 5-{2-[(3-chloro-4- fluorophenyl)amino]-4- (1H-indazol-1- yl)pyrimidin-5- yl}pyridine-3-carboxylic acid & 5-{2-[3-chloro-4- fluorophenyl)amino]-4- (2H-indazol-2- yl)pyrimidin-5- yl}pyridine-3-carboxylic acid | MS(ES): 459 (M − ) for C₂₃H₁₄ClFN₆O₂. 400 MHz, DMSO-d₆: δ 7.10 (t, J = 6.80 Hz, 1H), 7.25 (t, J = 8.80 Hz, 1H), 7.35-7.47 (m, 4H), 7.60 (t, J = 7.20 Hz, 1H), 7.66- 7.68 (m, 1H), 7.81 (d, J = 8.40 Hz, 2H), 7.85 (d, J = 7.60 Hz, 1H), 8.03-8.10 (m, 3H), 8.18 (s, 1H), 8.20 (d, J = 2.40 Hz, 1H), 8.49 (br d, 1H), 8.53 (d, J = 2.00 Hz, 1H), 8.63 (d, J = 2.00 Hz, 1H), 8.73 (s, 1H), 8.87 (s, 1H), 8.97-8.99 (m, 2H), 10.27 (br s, 1H), 10.48 (br s, 1H). | Example 519 ethyl 5-{2-[(3- chloro-4- fluorophenyl) amino]-4-(1H- indazol-1- yl)pyrimidin-5-ylpyridine- 3-carboxylate & ethyl 5-{2- [(3-chloro-4- fluorophenyl) amino]-4-(2H- indazol-2- yl)pyrimidin- 5-yl}pyridine- 3-carboxylate |
| Example 529^{a)} | 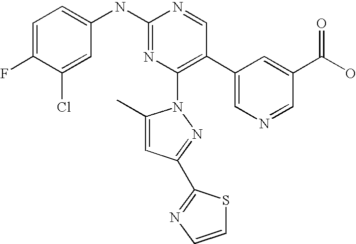 5-{2-[(3-chloro-4- fluorophenyl)amino]-4-[5- methyl-3-(1,3-thiazol-2- yl)-1H-pyrazol-1- yl]pyrimidin-5- yl}pyridine-3-carboxylic acid. | MS(ES): 506 (M − 1) for C₂₃H₁₅ClFN₇O₂S. 400 MHz, DMSO-d₆: δ 2.54 (s, 3H), 6.76 (s, 1H), 7.42 (t, J = 9.08 Hz, 1H), 7.63-7.68 (m, 2H), 7.82 (d, J = 3.20 Hz, 2H), 8.00 (t, J = 2.08 Hz, 1H), 8.09 (dd, J = 2.36, 6.74 Hz, 1H), 8.54 (d, J = 2.16 Hz, 1H), 8.90 (s, 1H), 8.93 (d, J = 1.88 Hz, 1H), 10.36 (s, 1H). | Example 520 ethyl 5-{2-[(3- chloro-4- fluorophenyl)- amino]-4-[5- methyl-3-(1,3- thiazol-2-yl)- 1H-pyrazol-1- yl]pyrimidin- 5-yl}pyridine- 3-carboxylate |
| Example 530^{b)} | 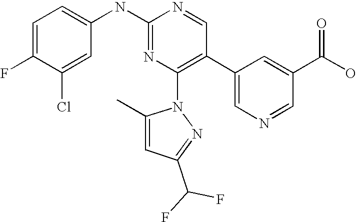 5-{2-[(3-chloro-4- fluorophenyl)amino]-4-[3- (difluoromethyl)-5-methyl- 1H-pyrazol-1- yl]pyrimidin-5- yl}pyridine-3-carboxylic acid | MS(ES): 475 (M + 1) for C₂₁H₁₄ClF₃N₆O₂. 400 MHz, DMSO-d₆: δ 2.30 (s,3H), 6.51 (s, 1H), 6.75 (t, J = 54.28 Hz, 1H), 7.39 (t, J = 9.08 Hz, 1H), 7.63- 7.66 (m, 1H), 8.00- 8.06 (m, 3H), 8.87 (s, 1H), 8.88 (br s, 1H), 10.39 (br s, 1H). | Example 521 ethyl 5-{2-[(3- chloro-4- fluorophenyl) amino]-4-[3- (difluoro- methyl)-5- methyl- 1H-pyrazol-1- yl]pyrimidin- 5-yl}pyridine- 3-carboxylate |
| Example 531^{b)} | 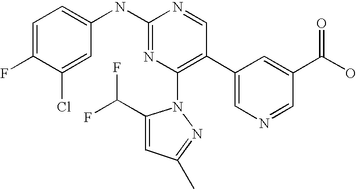 5-{2-[(3-chloro-4- fluorophenyl)amino]-4-[5- (difluoromethyl)-3-methyl- 1H-pyrazol-1- yl]pyrimidin-5- yl}pyridine-3-carboxylic acid | MS(ES): 475 (M + 1) for C₂₁H₁₄ClF₃N₆O_{2.} 400 MHz, DMSO-d₆: δ 1.98 (s, 3H), 6.71 (s, 1H), 7.40 (t, J = 8.80 Hz, 1H), 7.59 (t, J = 54.40 Hz, 1H), 7.61- 7.65 (m, 1H), 7.91 (br s, 1H), 8.05 (dd, J = 2.00, 6.60 Hz, 1H), 8.21(br s, 1H), 8.77 (s, 1H), 8.90 (br s, 1H), 10.22 (br s, 1H). | Example 522 ethyl 2-{2-[(3- chloro-4- fluorophenyl)- amino]-4-[5- (difluoro- methyl)-3- methyl- 1H-pyrazol-1- yl]pyrimidin- 5-yl}pyridine- 3-carboxylate |
| Example 532^{b)} | 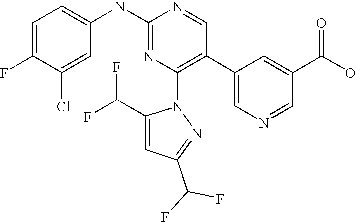 5-{4-[3,5- bis(difluoromethyl)-1H- pyrazol-1-yl]-2-[(3-chloro- 4- fluorophenyl)amino]- pyrimidin-5-yl}pyridine-3- carboxylic acid | MS(ES): 511 (M + 1) for C₂₁H₁₂ClF₅N₆O₂. 400 MHz, DMSO-d₆: δ 6.82 (t, J = 53.76 Hz, 1H), 7.23 (s, 1H), 7.41 (t, J = 9.12 Hz, 1H), 7.57 (t, J = 55.52 Hz, 1H), 7.63-7.66 (m, 1H), 7.90 (s, 1H), 8.03 (d, J = 4.92 Hz, 1H), 8.39 (br s, 1H), 8.92 (s, 2H), 10.39 (br s, 1H). | Example 523 ethyl 5-{4- [3,5- bis(difluoro- methyl)-1H- pyrazol-1-yl]- 2-[(3-chloro- 4- fluorophenyl) amino] pyrimidin-5- yl}pyridine-3- carboxylate |
| Example 533^{c)} | 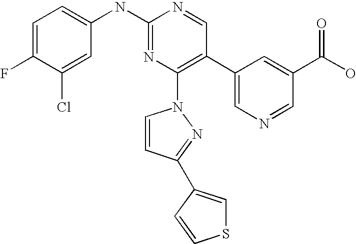 5-{2-[(3-chloro-4- fluorophenyl)amino]-4-[3- (thiophen-3-yl)-1H- pyrazol-1-yl]pyrimidin-5- yl}pyridine-3-carboxylic acid | MS(ES): 491 (M − 1) for C₂₃H₁₄ClFN₆O₂S. 400 MHz, DMSO-d₆: δ 6.91 (d, J = 4.52 Hz, 1H), 6.95 (br s, 1H), 7.43 (t, J = 8.92 Hz, 1H), 7.52 (br s, 1H), 7.62 (br s, 1H), 7.73- 7.75 (m, 1H), 8.09 (d, J = 5.20 Hz, 1H), 8.17 (br s, 1H), 8.47 (br s, 1H), 8.60 (s, 1H), 8.62 (s, 1H), 9.03 (s, 1H), 10.27 (s, 1H). | Example 524 ethyl 5-{2-[(3- chloro-4- fluorophenyl)- amino]-4-[3- (thiophen-3- yl)-1H- pyrazol-1- yl]pyrimidin- 5-yl}pyridine- 3-carboxylate |
| Example 534^{b)} | 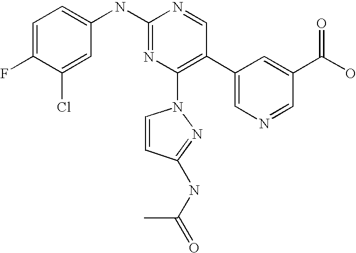 5-{4-[3-(acetylamino)-1H- pyrazol-1-yl]-2-[(3-chloro- 4- fluorophenyl)amino] pyrimidin-5-yl}pyridine-3- carboxylic acid | MS(ES): 468 (M + 1) for C₂₁H₁₅ClFN₇O₃. 400 MHz, DMSO-d₆: δ 1.92 (s, 3H), 6.83 (d, J = 2.20 Hz, 1H), 7.37 (t, J = 9.04 Hz, 1H), 7.71 (dd, J = 2.60, 8.36 Hz, 1H), 8.02- 8.04 (m, 2H), 8.25 (br s, 1H), 8.33 (br s, 1H), 8.57 (br s, 1H), 8.93 (br s, 1H), 10.21 (br s, 1H), 10.42 (br s, 1H). | Example 525 ethyl 5-{4-[3- (acetylamino)- 1H-pyrazol-1- yl]-2-[(3- chloro-4- fluorophenyl) amino] pyrimidin-5- yl}pyridine-3- carboxylate |
| Example 535^{a)} | 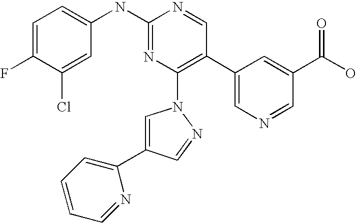 ethyl 5-{2-[(3-chloro-4- fluorophenyl)amino]-4-[4- (pyridin-2-yl)-1H-pyrazol- 1-yl]pyrimidin-5- yl}pyridine-3-carboxylate | MS(ES): 488 (M + 1) for C₂₄H₁₅ClFN₇O₂. 400 MHz, DMSO-d₆: δ 7.28-7.29 (m, 1H), 7.43 (t, J = 9.08 Hz, 1H), 7.71 (ddd, J = 2.76, 4.14, 9.06 Hz, 1H), 7.78 (d, J = 7.88 Hz, 1H), 7.84 (td, J = 1.72, 10.72 Hz, 1H), 8.13 (d, J = 2.64 Hz, 1H), 8.15 (t, J = 1.92 Hz, 1H), 8.18 (s, 1H), 8.58 (d, J = 4.16 Hz, 1H), 8.69 (d, J = 2.20 Hz, 1H), 8.72 (s, 1H), 9.00 (d, J = 1.96 Hz, 1H), 9.03 (s, 1H), 10.36 (s, 1H), 13.45 (br s, 1H). | Example 526 ethyl 5-{2-[(3- chloro-4- fluorophenyl) amino]-4-[4- (pyridin-2-yl)- 1H-pyrazol-1- yl]pyrimidin- 5-yl}pyridine- 3-carboxylate |
| Example 536^{a)} | 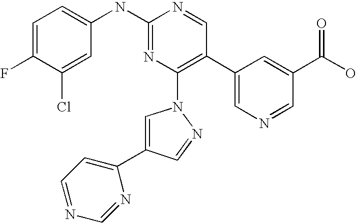 5-{2-[(3-chloro-4- fluorophenyl)amino]-4-[4- (pyrimidin-4-yl)-1H- pyrazol-1-yl]pyrimidin-5- yl}pyridine-3-carboxylic acid | MS(ES): 487 (M − 1) for C₂₃H₁₄ClFN₈O₂. 400 MHz, DMSO-d₆: δ 7.43 (t, J = 9.08 Hz, 1H), 7.70-7.74 (m, 1H), 7.88 (d, J = 5.28 Hz, 1H), 8.11-8.14 (m, 2H), 8.30 (s, 1H), 8.68 (d, J = 1.84 Hz, 1H), 8.76 (s, 1H), 8.80 (d, J = 5.24 Hz, 1H), 9.01 (d, J = 1.60 Hz, 1H), 9.15 (s, 1H), 9.20 (s,1H), 10.40 (s, 1H), 13.49 (br s, 1H). | Example 527 ethyl 5-{2-[(3- chloro-4- fluorophenyl) amino]-4-[4- (pyrimidin-4- yl)-1H- pyrazol-1- yl]pyrimidin- 5-yl}pyridine- 3-carboxylate |

^{a)}Method C
^{b)}Method D
^{c)}Method D, THF-H₂O

### General method for the synthesis of 5-{2-[(3-chloro-4-fluorophenyl)amino]-4-[azol-1-yl]pyrimidin-5-yl}pyridine-3-carboxamide

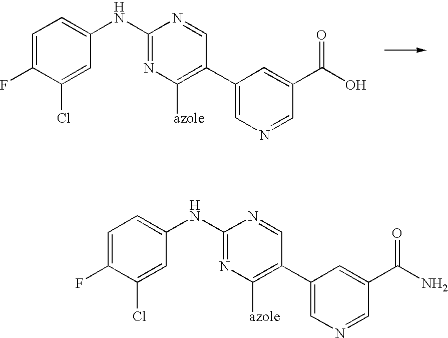

### Method E:

To a stirred solution of the carboxylic acid derivative (1 eq), pyridine (0.5 ml) and di-tert-butyl dicarbonate (1.3 eq) in dioxane (10-15 mL), ammonium hydrogencarbonate (1.26 eq) was added and the mixture was stirred for 4-16 h. The reaction mixture was then diluted with water (30-40 ml), stirred until precipitation was complete and the residue was then collected by filtration, washed with water, dried and further purified by column chromatography to give the product.

### Method F:

A solution of carboxylic acid derivative (1.02 mmol) taken in thionyl chloride (2 mL) was heated to 85° C. for 2 h. Thionyl chloride was then removed in vacuo and the solid obtained was quenched with a saturated solution of NH₃ in 1,4 dioxane (25 mL) and stirred for 20 min. The solid obtained was filtered and dried. The crude material was further purified by RP-HPLC to give the product.

The compounds in the below table were prepared using the methods described above as indicated and the starting material specified.

| Compound | Structure | Mass spectrum and ¹H NMR | SM |
|---|---|---|---|
| Example 537^{d)} | 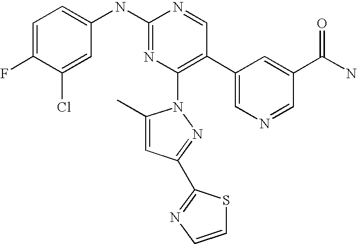 5-{2-[(3-chloro-4- fluorophenyl)amino]-4- [5-methyl-3-(1,3- thiazol-2-yl)-1H- pyrazol-1-yl]pyrimidin- 5-yl}pyridine-3- carboxamide | MS(ES): 505 (M '1 1) for C₂₃H₁₆ClFN₈OS. 400 MHz, DMSO-d₆: δ 2.49 (s, 3H), 6.77 (br s, 1H), 7.43 (t, J = 9.08 Hz, 1H), 7.58 (br s, 1H), 7.64 (d, J = 3.20 Hz, 1H), 7.66-7.68 (m, 1H), 7.83 (d, J = 3.24 Hz, 1H), 8.09- 8.13 (m, 3H), 8.31 (d, J = 2.08 Hz,1H), 8.89 (d, J = 1.96 Hz, 1H), 8.91 (s, 1H). | Example 529 5-{2-[(3- chloro-4- fluorophenyl)- amino]-4-[5- methyl-3-(1,3- thiazol-2-yl)- 1H-pyrazol-1- yl]pyrimidin-5- yl}pyridine-3- carboxylic acid |
| Example 538^{a)} | 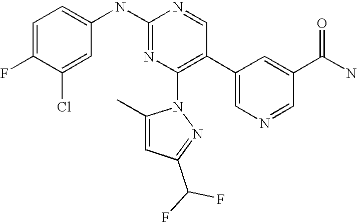 5-{2-[(3-chloro-4- fluorophenyl)amino]-4- [3-(difluoromethyl)-5- methyl-1H-pyrazol-1- yl]pyrimidin-5- yl}pyridine-3- carboxamide | MS(ES): 474 (M + 1) for C₂₁H₁₅ClF₃N₇O. 400 MHz, DMSO-d₆: δ 2.41 (s, 3H), 6.56 (s, 1H), 6.73 (t, J = 54.16 Hz, 1H),7.42 (t, J = 9.08 Hz, 1H), 7.61 (s, 1H), 7.65 (ddd, J = 2.72, 4.16, 9.03 Hz, 1H), 8.02 (t, J = 2.08 Hz, 1H), 8.07 (dd, J = 2.44, 6.62 Hz, 1H), 8.11 (s, 1H), 8.21 (d, J = 2.16 Hz, 1H), 8.90 (d, J = 1.96 Hz, 1H), 8.95 (s, 1H), 10.41 (s, 1H). | Example 550 5-{2-[(3- chloro-4- fluorophenyl) amino]-4-[3- (difluoromethyl)- 5-methyl-1H- pyrazol-1- yl]pyrimidin-5- yl}pyridine-3- carboxylic acid |
| Example 539^{a)} | 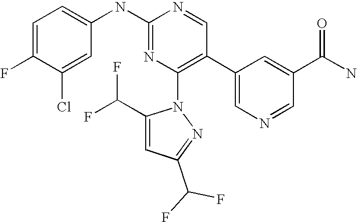 5-{4-[3,5- bis(difluoromnethyl)- 1H-pyrazol-1-yl]-2-[(3- chloro-4- fluorophenyl)amino] pyrimidin-5-yl}pyridine- 3-carboxamide | MS(ES): 510 (M + 1) for C₂₁H₁₃ClF₅N₇O. 400 MHz, DMSO-d₆: δ 6.82 (t, J = 53.76 Hz, 1H), 7.25 (s, 1H), 7.41 (t, J = 9.08 Hz, 1H), 7.58 (t, J = 53.68 Hz, 1H), 7.60-7.64 (m, 2H), 8.03 (br s, 1H), 8.04 (t, J = 2.08 Hz, 1H), 8.14 (br s, 1H), 8.30 (d, J = 2.08 Hz, 1H), 8.93 (d, J = 2.08 Hz, 1H), 8.94 (s, 1H), 10.40 (br s, 1H). | Example 532 5-{4-[3,5- bis(difluoro- methyl)-1H- pyrazol-1-yl]- 2-[(3-chloro-4- fluorophenyl) amino]pyrimidin- 5-yl}pyridine- 3-carboxylic acid |
| Example 540^{a)} | 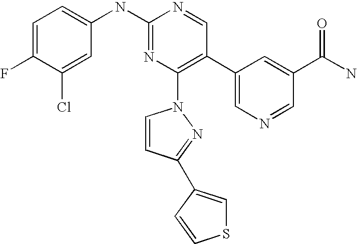 5-{2-[(3-chloro-4- fluorophenyl)amino]-4- [3-(thiophen-3-yl)-1H- pyrazol-1-yl]pyrimidin- 5-yl}pyridine-3- carboxamide | MS(ES): 492 (M + 1) for C₂₃H₁₅ClFN₇OS. 400 MHz, DMSO-d₆: δ 6.92 (dd, J = 1.08, 5.00 Hz, 1H), 6.97 (d, J = 2.72 Hz, 1H), 7.43 (t, J = 9.08 Hz, 1H), 7.52 (dd, J = 2.92, 4.96 Hz, 1H), 7.62 (br s, 1H), 7.66 (dd, J = 1.16, 2.86 Hz, 1H), 7.72- 7.76 (m, 1H), 8.08 (dd, J = 2.48, 6.74 Hz, 1H), 8.17 (br s, 1H), 8.22 (t, J = 2.00 Hz, 1H), 8.50 (d, J = 2.68 Hz, 1H), 8.60 (br s, 1H), 8.64 (s, 1H), 9.01 (br s, 1H). | Example 533 5{2-[(3- choloro-4- fluorophenyl) amino]-4-[3- (thiophen-3- yl)-1H-pyrazol- 1-yl]pyrimidin- 5-yl}pyridine- 3-carboxylic acid |
| Example 541^{a)} | 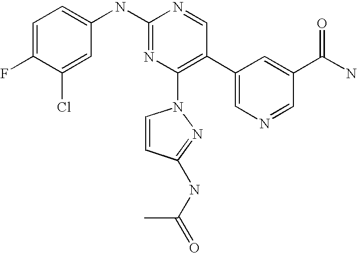 5-{4-[3-(acetylamino)- 1H-pyrazol-1-yl]-2-[(3- chloro-4- fluorophenyl)amino] pyrimidin-5-yl}pyridine- 3-carboxamide | MS(ES): 467 (M + 1) for C₂₁H₁₆ClFN₈O₂. 400 MHz, DMSO-d₆: δ 1.94 (s, 3H), 6.88 (br s, 1H), 7.41 (t, J = 9.12 Hz, 1H), 7.62 (br s, 1H), 7.71-7.74 (m, 1H), 8.05 (d, J = 4.56 Hz, 1H), 8.14-8.15 (m, 2H), 8.33 (d, J = 2.16 Hz, 1H), 8.43 (br s, 1H), 8.66 (br s, 1H), 8.95 (br s, 1H), 10.28 (s, 1H), 10.33 (s, 1H). | Example 534 (PE-032-019) 5-{4-[3- (acetylamino)- 1H-pyrazol-1- y]-2-[(3- chloro-4- fluorophenyl) amino]pyrimidin- 5-yl}pyridine- 3-carboxylic acid |
| Example 542^{d)} | 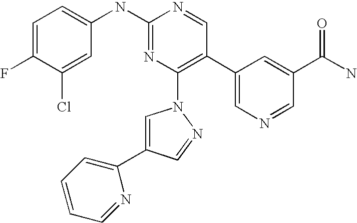 5-{2-[(3-chloro-4- fluorophenyl)amino]-4- [(4-(pyridin-2-yl)-1H- pyrazol-1-yl]pyrimidin- 5-yl}pyridine-3- carboxamide | MS(ES): 487 (M + 1) for C₂₄H₁₆ClFN₈O. 400 MHz, DMSO-d₆: δ 7.27 (t, J = 6.12 Hz, 1H), 7.43 (t, J = 9.12 Hz, 1H), 7.61 (br s, 1H), 7.70-7.73 (m, 1H), 7.77- 7.84 (m, 2H), 8.13-8.18 (m, 4H), 8.54 (d, J = 1.96 Hz, 1H), 8.58 (d, J = 4.64 Hz, 1H), 8.72 (s, 1H), 8.95 (d, J = 1.84 Hz, 1H), 9.02 (s, 1H), 10.40 (br s, 1H). | Example 535 5-{2-[(3- chloro-4- fluorophenyl) amino]-4-[4- (pyridin-2-yl)- 1H-pyrazol-1- yl]pyrimidin-5- yl}pyridine-3- carboxylic acid |
| Example 543^{b)} | 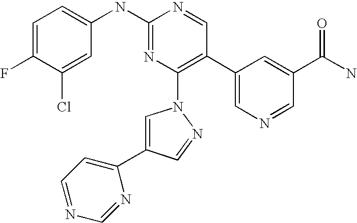 5-{2-[(3-chloro-4- fluorophenyl)amino]-4- [4-(pyrimidin-4-yl)- 1H-pyrazol-1- yl]pyrimidin-5- yl}pyridine-3- carboxamide | MS(ES): 486 (M − 1) and 973 (2M − 1) for C₂₃H₁₅ClFN₉O. 400 MHz, DMSO-d₆: δ 7.43 (t, J = 9.08 Hz, 1H), 7.62 (br s, 1H), 7.71-7.73 (m, 1H), 7.89 (d, J = 5.12 Hz, 1H), 8.12-8.15 (m, 3H), 8.30 (s, 1H), 8.54 (br s, 1H), 8.76 (s, 1H), 8.80 (d, J = 5.16 Hz, 1H), 8.96 (br s, 1H), 9.14 (s, 1H), 9.20 (s, 1H), 10.41 (br s, 1H). | Example 536 5-{2-[(3- chloro-4- fluorophenyl) amino]-4-[4- (pyrimidin-4- yl)-1H-pyrazol- 1-yl]pyrimidin- 5-yl}pyridine- 3-carboxylic acid |
| Example 544^{c)} | 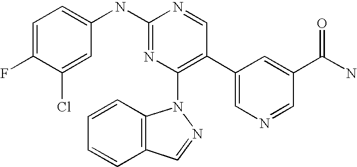 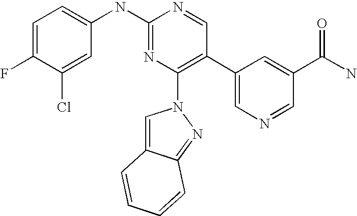 5-{2-[(3-chloro-4- fluorophenyl)amino]-4- (1H-indazol-1- yl)pyrimidin-5- yl}pyridine-3- carboxamide & 5-{2- [(3-chloro-4- fluorophenyl)amino]-4- (2H-indazol-2- yl)pyrimidin-5- yl}pyridine-3- carboxamide | MS(ES): 460 (M + 1) for C₂₃H₁₅ClFN₇O. ¹H NMR 400 MHz, DMSO- d₆: δ 7.07-7.13 (m, 1H), 7.22- 7.28 (m, 1H), 7.34-7.40 (m, 2H), 7.40-7.47 (m, 1H),7.56- 7.63 (m, 3H), 7.67 (d, J = 8.55 Hz, 1H), 7.76-7.83 (m, 1H), 7.86 (d, J = 7.93 Hz, 1H), 8.06-8.16 (m, 4H), 8.17- 8.23 (m, 3H), 8.32 (d, J = 1.83 Hz, 1H), 8.47 (d, J = 1.83 Hz, 3H), 8.73 (s, 1H), 8.85-8.90 (m, 3H), 10.27 (s, 1H), 10.49 (s, 1H). | Example 528 5-{2-[(3- chloro-4- fluorophenyl)- amino]-4-(1H- indazol-1- yl)pyrimidin-5- yl}pyridine-3- carboxylic acid & 5-{2-[(3- chloro-4- fluorophenyl) a (2H- indazol-2- yl)pyrimidin-5- yl}pyridine-3- carboxylic acid |

Solvents used in the reaction
a)Method E
^{b)}Method E; Boc₂O( 2.5 eq), Py (4 eq), NH₄HCO₃ (4 eq), DMF
^{c)}Method E; DMSO
^{d)}Method F

### General method for the synthesis of 5-{2-[(3-chloro-4-fluorophenyl)amino]-4-[azol-1-yl]pyrimidin-5-yl}-N-methoxypyridine-3-carboxamide

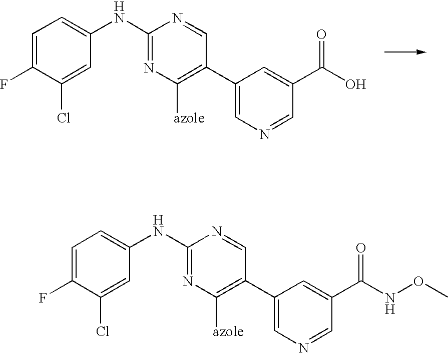

### Method G:

To a mixture of carboxylic acid derivative (1 eq), triethylamine (3 eq) and methoxylamine hydrochloride (2 eq) in DCM was added T3P (50% in EtOAc, 1.5 eq) slowly at 0° C. The reaction mixture was slowly raised to room temperature and stirred overnight. The reaction mixture was then diluted with dichloromethane (12 mL) and washed successively with water, 10% aq sodium bicarbonate solution and brine. The organic layer was dried over sodium sulphate, filtered, concentrated and further purified by silica gel column chromatography to yield the product.

### Method H:

To a mixture of carboxylic acid derivative (1 eq) and HATU (1.5 eq) in NMP, was added triethylamine (3 eq) and methoxylamine hydrochloride (1.2 eq) and the reaction mixture was stirred overnight. The reaction mixture was then diluted with water and the aqueous layer was extracted with EtOAc. The organic layer successively washed with 10% aq sodium bicarbonate solution and brine. The organic layer was dried over sodium sulphate, filtered, concentrated and further purified by silica gel column chromatography to yield the product.

The compounds in the below table were prepared using the methods described above as indicated and the starting material specified.

| Compound | Structure | Mass and ¹H NMR data | SM |
|---|---|---|---|
| Example 545^{a)} | 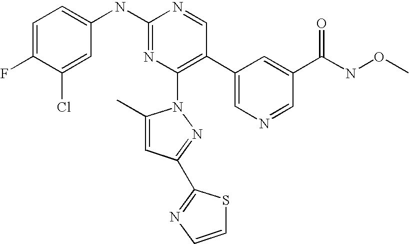 5-{2-[(3-chloro-4- fluorophenyl)amino]-4- [5-methyl-3-(1,3-thiazol- 2-yl)-1H-pyrazol-1- yl]pyrimidin-5-yl}-N- methoxypyridine-3- carboxamide | MS(ES): 537 (M + 1) for C₂₄H₁₈ClFN₈O₂S. 400 MHz, DMSO-d₆: δ 2.51 (s, 3H), 3.67 (s, 3H), 6.77 (s, 1H), 7.43 (t, J = 9.12 Hz, 1H), 7.64-7.68 (m, 2H), 7.83 (d, J = 3.24 Hz, 1H), 7.99 (br s, 1H), 8.09 (dd, J = 2.36, 6.68 Hz, 1H), 8.35 (d, J = 1.88 Hz, 1H), 8.76 (d, J = 1.92 Hz, 1H), 8.90 (s, 1H), 10.39 (s, 1H), 11.92 (br s, 1H). | Example 529 5-{2-[(3- chloro-4- fluorophenyl) amino]-4-[5- methyl-3-(1,3- thiazol-2-yl)- 1H-pyrazol-1- yl]pyrimidin- 5-yl}pyridine- 3-carboxylate acid |
| Example 546^{a)} | 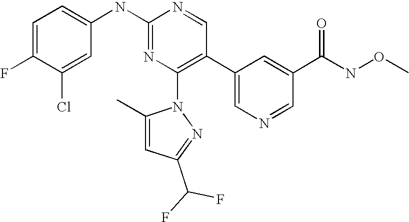 5-{2-[(3-chloro-4- fluorophenyl)amino]-4- [3-(difluoromethyl)-5- methyl-1H-pyrazol-1- yl]pyrimidin-5-yl}-N- methoxypyridine-3- carboxamide | MS(ES): 504 (M + 1) for C₂₂H₁₇ClF₃N₇O₂. 400 MHz, DMSO-d₆: δ 2.41 (s, 3H), 3.71 (s, 3H), 6.57 (s, 1H), 6.73 (t, J = 54.12 Hz, 1H), 7.42 (t, J = 9.12 Hz, 1H), 7.64-7.66 (m, 1H), 7.88 (s, 1H), 8.06-8.07 (m, 1H), 8.26 (d, J = 1.56 Hz, 1H), 8.76 (d, J = 1.92 Hz, 1H), 8.95 (s, 1H), 10.46 (s, 1H), 11.95 (br s, 1H). | Example 530 5-{2-[(3- chloro-4- fluorophenyl) amino]-4-[3- (difluoromethyl)- 5-methyl- 1H-pyrazol-1- yl]pyrimidin- 5-yl}pyridine- 3-carboxylic acid |
| Example 547^{a)} | 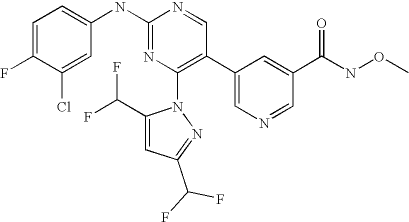 5-{4-[3,5- bis(difluoromethyl)-1H- pyrazol-1-yl]-2-[(3- chloro-4- fluorophenyl)amino] pyrimidin-5-yl}-N- methoxypyridine-3- carboxamide | MS(ES): 540 (M + 1) for C₂₂H₁₅ClF₅N₇O₂. 400 MHz, DMSO-d₆: δ 3.71 (s, 3), 6.82 (t, J = 54.04 Hz, 1H), 7.26 (s, 1H), 7.41 (t, J = 9.16 Hz, 1H), 7.59-7.72 (m, 2H), 7.93 (br s, 1H), 8.03 (br s, 1H), 8.35 (br s, 1H), 8.81 (br s, 1H), 8.94 (br s, 1H), 10.41 (br s, 1H), 11.98 (br s, 1H). | Example 532 5-{4-[3,5- bis(difluoro- methyl)-1H- pyrazol-1-yl]- 2-[(3-chloro-4- fluorophenyl) amino] pyrimidin-5- yl}pyridine-3- carboxylic acid |
| Example 548^{a)} | 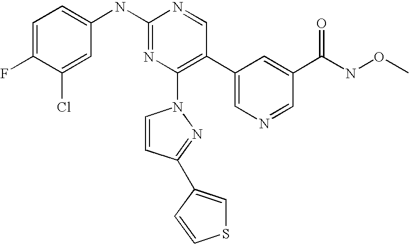 5-{2-[(3-chloro-4- fluorophenyl)amino]-4- [3-(thiophen-3-yl)-1H- pyrazol-1-yl]pyrimidin- 5-yl}-N- methoxypyridine-3- carboxamide | MS(ES): 520 (M − 1) for C₂₄H₁₇ClFN₇O₂S. 400 MHz, DMSO-d₆: δ 3.70 (s, 3H), 6.93 (dd, J = 1.04, 5.02 Hz, 1H), 6.98 (d, J = 2.68 Hz, 1H), 7.43 (t, J = 9.08 Hz, 1H), 7.52 (dd, J = 2.92, 4.96 Hz, 1H), 7.65 (dd, J = 1.08, 2.82 Hz, 1H), 7.72-7.76 (m, 1H), 8.08 (dd, J = 2.52, 6.74 Hz, 1H), 8.;13 (br t, 1H), 8.51 (d, J = 2.72 Hz,1H), 8.63 (br s, 2H), 8.89 (br s, 1H), 10.28 (s, 1H), 12.00 (br, s 1H). | Example 533 5-{2-[(3- chloro-4- fluorophenyl) amino]-4-[3- (thiophen-3- yl)-1H- pyrazol-1- yl]pyrimidin- 5-yl}pyridine- 3-carboxylic acid |
| Example 549^{b)} | 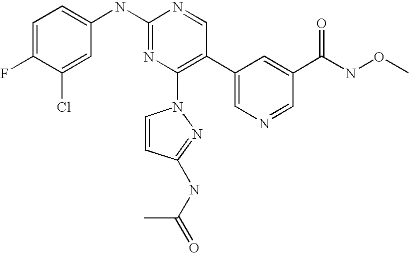 5-{4-[3-(acetylamino)- 1H-pyrazol-1-yl]-2-[(3- chloro-4- fluorophenyl)amino]- pyrimidin-5-yl}-N- methoxypyridine-3- carboxamide | MS(ES): 497 (M + 1) for C₂₂H₁₈ClFN₈O₃. 400 MHz, DMSO-d₆: δ 1.94 (s, 3H), 3.73 (s, 3H), 6.87 (d, J = 2.16 Hz, 1H), 7.41 (t, J = 9.12 Hz, 1H), 7.71-7.75 (m, 1H), 8.04- 8.05 (m, 2H), 8.33 (d, J = 2.40 Hz, 1H), 8.45 (br s, 1H), 8.65 (s, 1H), 8.83 (br s, 1H), 10.28-10.31 (m, 2H), 11.96 (br s, 1H). | Example 534 5-{4-[3- (acetylamino)- 1H-pyrazol-1- yl]-2-[(3- chloro-4- fluorophenyl) amino] pyrimidin-5- yl}pyridine-3- carboxylic acid |
| Example 550^{b)} | 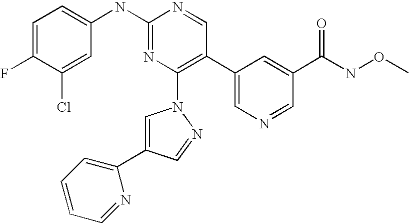 5-{2-[(3-chloro-4- fluorophenyl)amino]-4- [4-(pyridin-2-yl)-1H- pyrazol-1-yl]pyrimidin- 5-yl}-N- methoxypyridine-3- carboxamide | MS(ES): 517 (M + 1) for C₂₅H₁₈ClFN₈O₂₂. 400 MHz, DMSO-d₆: δ 3.72 (s, 3H), 7.27 (t, J = 5.92 Hz, 1H), 7.44 (t, J = 9.16 Hz, 1H), 7.70-7.73 (m, 1H), 7.78 (d, J = 7.48 Hz, 1H), 7.84 (t, J = 7.32 Hz, 1H), 8.06 (br s, 1H), 8.14 (dd, J = 2.44, 6.78 Hz, 1H), 8.18 (s, 1H), 8.56-8.58 (m, 2H), 8.71 (s, 1H), 8.83 (s, 1H), 9.03 (s, 1H), 10.38 (s, 1H), 11.98 (br s, 1H). | Example 535 5-{2-[(3- chloro-4- fluorophenyl) amino]-4-[4- (pyridin-2-yl)- 1H-pyrazol-1- yl]pyrimidin- 5-yl}pyridine- 3-carboxylic acid |
| Example 551^{a)} | 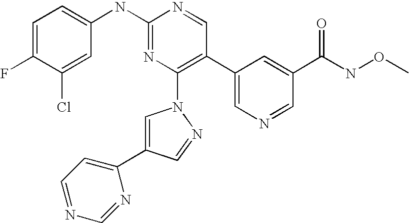 5-{2-[(3-chloro-4- fluorophenyl)amino]-4- [4-(pyrimidin-4-yl)-1H- pyrazol-1-yl]pyrimidin- 5-yl}-N- methoxypyridine-3- carboxamide | MS(ES): 518 (M + 1) for C₂₄H₁₇ClFN₉O₂. 400 MHz, DMSO-d₆: δ 3.72 (s, 3H), 7.43 (t, J = 9.08 Hz, 1H), 7.71-7.74 (m, 1H), 7.89 (d, J = 4.76 hz, 1H), 8.06 (s, 1H), 8.12 (dd, J = 2.48, 6.68 Hz, 1H), 8.30 (s, 1H), 8.56 (d, J = 1.52 Hz, 1H), 8.75 (s, 1H), 8.80-8.83 (m, 2H), 9.15 (s, 1H), 9.20 (s, 1H), 10.41 (s, 1H), 11.97 (s, 1H). | Example 536 5-{2-[(3- chloro-4- fluorophenyl) amino]-4-[4- (pyrimidin-4- yl)-1H- pyrazol-1- yl]pyrimidin- 5-yl}pyridine- 3-carboxylic acid |
| Example 552^{a)} | 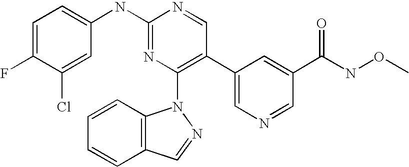 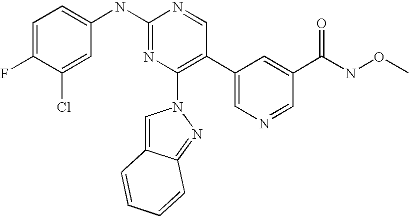 5-{2-[(3-chloro-4- fluorophenyl)amino]-4- (1H-indazol-1- yl)pyrimidin-5-yl}-N- methoxypyridine-3- carboxamide & 5-{2-[(3- chloro-4- fluorophenyl)amino]-4- (2H-indazol-2- yl)pyrimidin-5-yl}-N- methoxypyridine-3- carboxamide> | MS(ES): 490 (M + 1) for C₂₄H₁₇ClFN₇O₂. ¹H NMR 400 MHz, DMSO-d₆: δ 3.71 (s, 6H), 7.07-7.13 (m, 1H), 7.22- 7.28 (m, 1H), 7.34-7.40 (m, 2H), 7.41 (s, 1H), 7.41-7.48 (m, 1H), 7.57- 7.63 (m, 1H), 7.63-7.69 (m, 1H), 7.80 (d, J = 8.80 Hz, 1H),7.76-7.78 (m, 1H), 7.85 (d, J = 7.83 Hz, 1H), 7.98 (br s, 1H), 8.02-8.06 (m, 1H), 8.02- 8.05 (m, 1H), 8.07 (br s, 1H), 8.18 (s, 1H), 8.19 (d, J = 2.45 Hz, 1H), 8.34 (d, J = 1.47 hz, 1H), 8.48 (d, J = 1.71 Hz, 2H), 8.71 (s, 1H), 8.79 (s, 2H), 8.85 (s, 1H), 8.98 (s, 1H), 10.27 (s, 1H), 10.48 (s, 1H), 11.94 (br s, 1H). | Example 528 5-{2-[(3- chloro-4- fluorophenyl) amino]-4-(1H- indazol-1- yl)pyrimidin- 5-yl}pyridine- 3-carboxylic acid & 5-{2- [(3-chloro-4- fluorophenyl) amino]-4-(2H- indazol-2- yl)pyrimidin- 5-yl}pyridine- 3-carboxylic acid |

### General method for the synthesis of ethyl (2E)-3-(3-{2-[(3-chloro-4-fluorophenyl)amino]-4-(azol-1-yl)pyrimidin-5-yl}phenyl)prop-2-enoate

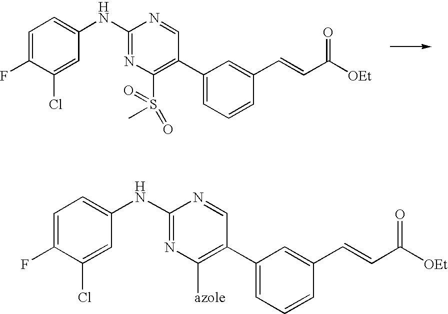

### Method I:

A solution of azole (2.2 eq) in DMF (1 mL) was added slowly to a suspension of sodium hydride (2.1 eq) in DMF (1 mL). The reaction mixture was stirred for 30 min at room temperature. A solution of (E)-ethyl 3-(3-(2-(3-chloro-4-fluorophenylamino)-4-(methylsulfonyl)pyrimidin-5-yl)phenyl)acrylate Intermediate 125 (1 eq) in DMF (1 mL) was added slowly to the reaction mixture at 0° C. with stirring and allowed to warm to ambient temperature over 2.5 h. Water was added (˜6 mL) and the solid formed was filtered, dried to yield the product.

### Method J:

A solution of azole (2.2 eq) in DMSO (1 mL) was added slowly to a suspension of sodium hydride (2.1 eq) in DMSO (1 mL). The reaction mixture was stirred for 30 min at room temperature. A solution of (E)-ethyl 3-(3-(2-(3-chloro-4-fluorophenylamino)-4-(methylsulfonyl)pyrimidin-5-yl)phenyl)acrylate Intermediate 125 (1 eq) in DMSO (1 mL) was added slowly to the reaction mixture at 0° C. with stirring and allowed to warm to ambient temperature over 2.5 h. Water was added (˜6 mL) and the solid formed was filtered, dried to yield the product.

### Method K:

A suspension of azole (1.2 eq), potassium tert-butoxide (1.5 eq) and (E)-ethyl 3-(3-(2-(3-chloro-4-fluorophenylamino)-4-(methylsulfonyl)pyrimidin-5-yl)phenyl)acrylate Intermediate 125 (1 eq) in DMSO (3 mL) was subjected to microwave irradiation at 130° C. for 1 h. After the reaction cooled to RT, the mixture was diluted with EtOAc, washed successively with water and brine. The organic layer was dried over sodium sulphate, filtered, concentrated and further purified by silica gel column chromatography to yield the product.

The compounds in the below table were prepared using the methods described above as indicated and the starting material specified.

| Compound | Structure | Mass spectrum and ¹H NMR | SM |
|---|---|---|---|
| Example 553^{a)} | 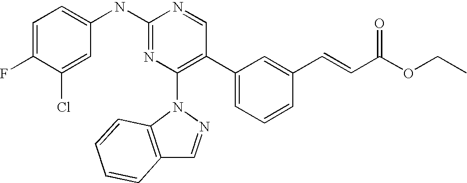 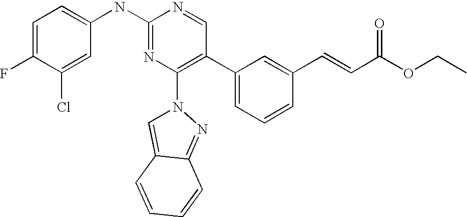 ethyl (2E)-3-(3-{2-[(3- chloro-4- fluorophenyl)amino]-4-(1H- indazol-1-yl)pyrimidin-5- yl}phenyl)prop-2-enoate & ethyl (2E)-3-(3-{2-[(3- chloro-4- fluorophenyl)amino]-4-(2H- indazol-2-yl)pyrimidin-5- yl}phenyl)prop-2-enoate | ¹H NMR 400 MHz, DMSO- d₆:: δ 1.24 (t, J = 7.03 Hz, 3H), 4.16 (d, J = 7.03 Hz, 2H), 6.51 (d, J = 16.06 Hz, 1H), 6.58 (d, J = 16.06 Hz, 1H), 7.00 (d, J = 7.03 Hz, 1H), 7.08 (t, J = 7.53 Hz, 2H), 7.21-7.48 (m, 8H), 7.52-7.68 (m, 6H), 7.72-7.79 (m, 2H), 7.83 (d, J = 7.28 Hz, 1H), 7.94 (s, 1H), 8.10 (dd, J = 6.90, 2.38 Hz, 1H), 8.17 (s, 1H), 8.17-8.21 (m, 1H), 8.28- 8.34 (m, 1H), 8.73 (s, 1H), 8.80 (s, 1H), 8.87 (d, J = 1.00 Hz, 1H), 10.21 (s, 1H), 10.43 (s, 1H). | 1H- indazole |
| Example 554^{b)} | 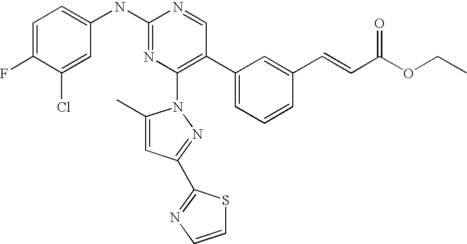 ethyl (2E)-3-(3-{2-[(3- chloro-4- fluorophenyl)amino]-4-[5- methyl-3-(1,3-thiazol-2-yl)- 1H-pyrazol-1-yl]pyrimidin- 5-yl}phenyl)prop-2-enoate | MS(ES): 561 (M + 1) for C₂₈H₂₂ClFN₆O₂S. 300 MHz, DMSO-d₆: δ 1.22 (t, J = 6.90 Hz, 3H), 2.27 (s, 3H), 4.15 (q, J = 7.05 Hz, 2H), 6.56 (d, J = 16.05 Hz, 1H), 6.72 (s, 1H), 7.03 (d, J = 7.92 Hz, 1H), 7.32 (t, J = 7.32 Hz, 1H), 7.41 (t, J = 9.21 Hz, 1H), 7.53-7.66 (m, 5H), 7.83 (d, J = 3.15 Hz, 1H), 8.11 (d, J = 6.39 Hz, 1H), 8.92 (s, 1H), 10.39 (br s, 1H). | 2-(5- methyl- 1H- pyrazol-3- yl)-1,3- thiazole |
| Example 555^{b)} | 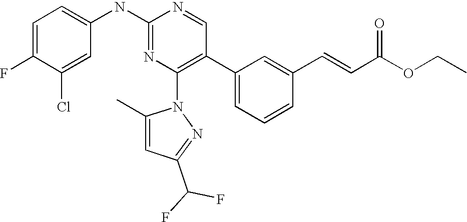 ethyl (2E)-3-(3-{2-[(3- chloro-4- fluorophenyl)amino]-4-[3- (difluoromethyl)-5-methyl- 1H-pyrazol-1-yl]pyrimidin- 5-yl}phenyl)prop-2-enoate | MS(ES): 528 (M + 1) for C₂₆H₂₁ClF₃N₅O₂. 300 MHz, DMSO-d₆: δ 1.24 (t, J = 7.89 Hz, 3H), 2.20 (s, 3H), 4.18 (q, J = 7.44 Hz, 2H), 6.51 (s, 1H), 6.57 (d, J = 15.84 Hz, 1H), 6.82 (t, 1H), 6.97 (d, J = 7.86 Hz, 1H), 7.32 (t, J = 7.38 Hz, 1H), 7.41 (t, J = 9.18 Hz, 1H), 7.43 (br s, 1H), 7.56 (d, J = 16.11 Hz, 1H), 7.61-7.69 (m, 2H), 8.08 (dd, J = 8.76, Hz, 1H), 8.96 (s, 1H), 10.42 (s, 1H). | 3- (difluoro methyl)- 5-methyl- 1H- pyrazole |
| Example 556^{c)} | 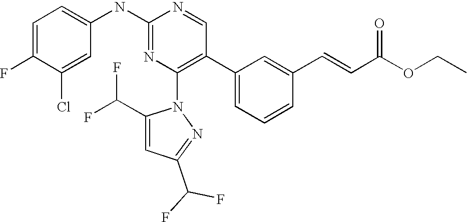 ethyl (2E)-3-(3-{4-[3,5- bis(trifluoromethyl)-1H- pyrazol-1-yl]-2-[(3-chloro-4- fluorophenyl)amino]pyrimidin- 5-yl}phenyl)prop-2-enoate | Taken to the next step on the basis of LCMS. MS(ES): 564 (M + 1) for C₂₆H₁₉ClF₅N₅O₂. 91% pure by LCMS. | 3,5- bis(di- fluoromethyl)- 1H- pyrazole |
| Example 557^{b)} | 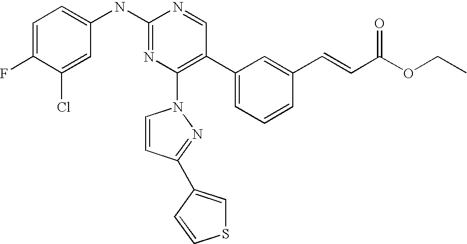 ethyl (2E)-3-(3-{2-[(3- chloro-4- fluorophenyl)amino]-4-[3- (thiophen-3-yl)-1H-pyrazol- 1-yl]pyrimidin-5- yl}phenyl)prop-2-enoate | MS(ES): 546 (M + 1) for C₂₈H₂₁ClFN₅O₂S. 400 MHz, DMSO-d₆: δ 1.23 (t, J = 7.04 Hz, 3H), 4.16 (q, J = 7.12 Hz, 2H), 6.64 (d, J = 16.04 Hz, 1H), 6.92 (d, J = 2.64 Hz, 1H), 6.98 (dd, J = 1.04, 5.00 Hz, 1H), 7.28 (d, J = 7.68 Hz, 1H), 7.41 (t, J = 7.92 Hz, 1H), 7.42 (t, J = 9.04 Hz, 1H), 7.50 (dd, J = 2.92, 5.00 Hz, 1H), 7.63-7.66 (m, 2H), 7.71-7.72 (m, 3H), 8.14 (dd, J = 2.60, 6.80 Hz, 1H), 8.32 (d, J = 2.68 Hz, 1H), 8.63 (s, 1H), 10.26 (s, 1H). | 3- (thiophen- 3-yl)-1H- pyrazole |
| Exampe\558^{a)} | 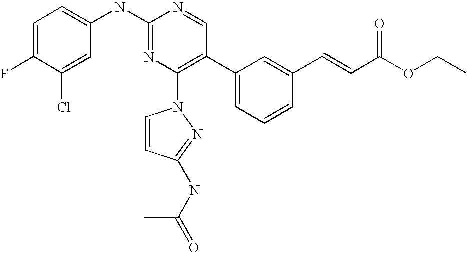 ethyl (2E)-3-(3-{4-[3- (acetylamino)-1H-pyrazol-1- yl]-2-[(3-chloro-4- fluorophenyl)amino]pyrimidin- 5-yl}phenyl)prop-2-enoate | MS(ES): 521 (M + 1) for C₂₆H₂₂ClFN₆O₃. 400 MHz, DMSO-d₆: δ 1.26 (t, J = 7.20 Hz, 3H), 1.96 (s, 3H), 4.19 (q, J = 7.20 Hz, 2H), 6.66 (d, J = 16.00 Hz, 1H), 6.83 (d, J = 2.40 Hz, 1H), 7.11 (d, J = 7.60 Hz, 1H), 7.37 (t, J = 8.00 Hz, 1H), 7.40 (t, J = 8.80 Hz, 1H), 7.63-7.70 (m, 3H), 7.72- 7.76 (m, 1H), 8.08 (dd, J = 2.80, 6.60 Hz, 1H), 8.15 (d, J = 2.40 Hz, 1H), 8.69 (s, 1H), 10.25 (s, 1H), 10.42 (s, 1H). | N-(1H- pyrazol-3- yl)acetamide |
| Example 559^{a)} | 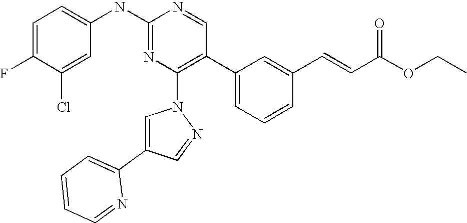 ethyl (2E)-3-(3-{2-[(3- chloro-4- fluorophenyl)amino]-4-[4- (pyridin-2-yl)-1H-pyrazol-1- yl]pyrimidin-5- yl}phenyl)prop-2-enoate | MS(ES): 541 (M + 1) for C₂₉H₂₂ClFN₆O₂. 400 MHz, DMSO-d₆: δ 1.24 (t, J = 7.2 Hz, 3H), 4.17 (q, J = 7.2 Hz, 2H), 6.64 (d, J = 16.4 Hz, 1H), 7.23 (t, J = 7.2 Hz, 1H), 7.27 (t, J = 6 Hz, 1H), 7.38 (t, J = 7.6 Hz, 1H), 7.43 (t, J = 9.6 Hz, 1H), 7.61-7.77 (m, 5H), 7.83 (t, J = 7.6 Hz, 1H), 8.16 (d, J = 6.8 Hz, 1H), 8.18 (s, 1H), 8.57 (d, J = 4.4 Hz, 1H), 8.73 (s, 1H), 8.91 (s, 1H), 10.32 (s, 1H). | 2-(1H- pyrazol-4- yl)pyridine |
| Example 560^{a)} | 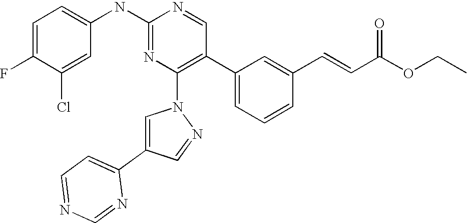 ethyl (2E)-3-(3-{2-[(3- chloro-4- fluorophenyl)amino]-4-[4- (pyrimidin-4-yl)-1H-pyrazol- 1-yl]pyrimidin-5- yl}phenyl)prop-2-enoate | MS(ES): 540 (M − 1) for C₂₈H₂₁ClFN₇O₂, 400 MHz, DMSO-d₆: δ 1.23 (t, J = 7.04 Hz, 3H), 4.16 (q, J = 7.12 Hz, 2H), 6.62 (d, J = 16.04 Hz, 1H), 7.21 (d, J = 7.64 Hz, 1H), 7.38 (t, J = 7.64 Hz, 1H), 7.42 (t, J = 9.20 Hz, 1H), 7.62 (d, J = 16.16 Hz, 1H), 7.68 (br s, 2H), 7.71-7.74 (m, 1H), 7.87 (dd, J = 8.40, 1.72 Hz, 1H), 8.14 (dd, J = 2.64, 6.74 Hz, 1H), 8.30 (s, 1H), 8.77 (s, 1H), 8.79 (d, J = 5.32 Hz, 1H), 9.09 (s, 1H), 9.13 (s, 1H), 10.36 (br s, 1H). | 4-(1H- pyrazol-4- yl)pyrimidine |

^{a)}Method I
^{b)}Method J
^{c)}Method K

### General method for the synthesis of (2E)-3-(3-{2-[(3-chloro-4-fluorophenyl)amino]-4-[azol-1-yl]pyrimidin-5-yl}phenyl)prop-2-enoic acid

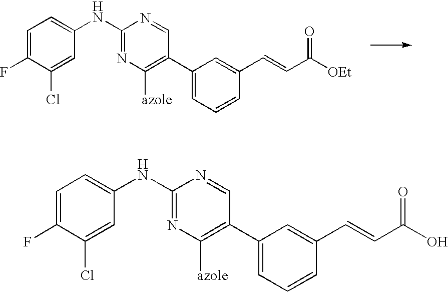

### Method L:

A solution of carboxylic ester derivative (1 eq) in a mixture of tetrahydrofuran (1 mL) and water (1 mL) was treated with 1 N aq. sodium hydroxide (4 eq) and allowed to stir at room temperature for 1 h. After completion of the reaction, the mixture was carefully acidified with 1 N HCl and the precipitate formed was filtered, washed with water and dried to the product.

### Method M:

A solution of carboxylic ester derivative (1 eq) in a mixture of dioxane (1 mL) and water (1 mL) was treated with 1 N aq. Barium hydroxide (2 eq) and warmed to 60° C. for 3-10 h. After completion of the reaction, the mixture was carefully acidified with 1 N HCl and the precipitate formed was filtered, washed with water, dried to yield the product.

| | | Mass spectrum and ¹H | |
|---|---|---|---|
| Compound | Structure | NMR | SM |
| Example 561^{a)} | 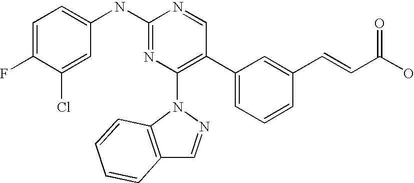 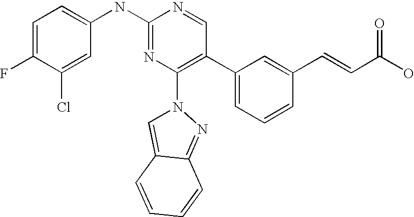 (2E)-3-(3-{2-[(3-chloro-4- fluorophenyl)amino]-4- (1H-indazol-1- yl)pyrimidin-5- yl}phenyl)prop-2-enoic acid & (2E)-3-(3-{2-[(3-chloro- 4-fluorophenyl)amino]-4- (2H-indazol-2- yl)pyrimidin-5- yl}phenyl)prop-2-enoic acid | MS(ES): 486 (M + 1) for C₂₆H₁₇ClFN₅O_{2.} ¹H NMR 400 MHz, DMSO-d₆: δ 6.43 (d, J = 16.00 Hz, 1H), 6.47 (d, J = 15.81 Hz, 1H), 6.98 (d, J = 7.53 Hz, 1H), 7.07 (t, J = 7.65 Hz, 2H), 7.22-7.30 (m, 3H), 7.32 (d, J = 8.03 Hz, 1H), 7.36 (d, J = 4.77 Hz, 1H), 7.37-7.45 (m, 3H), 7.45-7.49 (m, 1H), 7.49-7.53 (m, 1H), 7.53- 7.60 (m, 5H), 7.65 (dt, J = 3.36, 8.85 Hz, 1H), 7.73- 7.79 (m, 2H), 7.83 (d, J = 7.78 Hz, 1H), 8.11 (dd, J = 2.51, 6.78 Hz, 1H), 8.17- 8.21 (m, 1H), 8.31 (d, J = 8.28 Hz, 1H), 8.73 (s, 1H), 8.80 (s, 1H), 8.87 (s, 1H), 10.22 (s, 1H), 10.43 (s, 1H), 12.38 (br s, 1H). | Example 553 ethyl (2E)-3- (3-{2-[(3- chloro-4- fluorophenyl) amino]-4- (1H-indazol- 1- yl)pyrimidin- 5- yl}phenyl)prop- 2-enoate & ethyl (2E)-3- (3-{2-[(3- chloro-4- fluorophenyl) amino]-4- (2H-indazol- 2- yl)pyrimidin- 5- yl}phenyl)prop- 2-enoate |
| Example 562^{a)} | 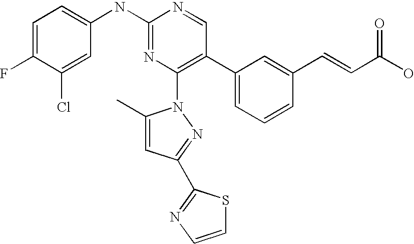 (2E)-3-(3-{2-[(3-chloro-4- fluorophenyl)amino]-4-[5- methyl-3-(1,3-thiazol-2- yl)-1H-pyrazol-1- yl]pyrimidin-5- yl}phenyl)prop-2-enoic acid | MS(ES): 531 (M − 1) for C₂₆H₁₈ClFN₆O₂S. 400 MHz, DMSO-d₆: δ 2.30 (s, 3H), 6.47 (d, J = 16.00 Hz, 1H), 6.72 (s, 1H), 7.02 (d, J = 7.92 Hz, 1H), 7.31 (t, J = 7.68 Hz, 1H), 7.41 (t, J = 9.12 Hz, 1H), 7.50 (d, J = 16.00 Hz, 1H), 7.55-7.56 (m, 2H), 7.64-7.65 (m, 2H), 7.83 (d, J = 3.24 Hz, 1H), 8.09 (dd, J = 38.52, 24.74 Hz, 1H), 8.91 (s, 1H), 10.37 (s, 1H), 12.37 (br s, 1H). | Example 554 ethyl (2E)-3- (3-{2-[(3- chloro-4- fluorophenyl) amino]-4-[5- methyl-3- (1,3-thiazol-2- yl)-1H- pyrazol-1- yl]pyrimidin- 5- yl}phenyl) prop-2-enoate |
| Example 563^{b)} | 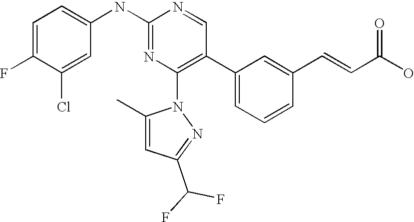 (2E)-3-(3-{2-[(3-chloro-4- fluorophenyl)amino]-4-[3- (difluoromethyl)-5-methyl- 1H-pyrazol-1- yl]pyrimidin-5- yl}phenyl)prop-2-enoic acid | MS(ES): 500 (M + 1) for C₂₄H₁₇ClF₃N₅O₂. 400 MHz, DMSO-d₆: δ 2.02 (s, 3H), 6.46 (d, J = 16.00 Hz, 1H), 6.52 (br s, 1H), 6.82 (t, J = 54.20 Hz, 1H), 6.99 (d, J = 7.80 Hz, 1H), 7.33 (t, J = 7.76 Hz, 1H), 7.37 (br s, 1H), 7.41 (t, J = 9.16 Hz, 1H), 7.49 (d, J = 16.00 Hz, 1H), 7.59 (d, J = 7.80 Hz, 1H), 7.64- 7.68 (m, 1H), 8.08 (dd, J = 1.88, 6.54 Hz, 1H), 8.95 (s, 1H), 10.42 (s, 1H), 12.46 (br s, 1H). | Example 555 ethyl (2E)-3- (3-{2-[(3- chloro-4- fluorophenyl)- amino]-4-[3- (difluoro- methyl)-5- methyl- 1H-pyrazol-1- yl]pyrimidin- 5- yl}phenyl)- prop-2-enoate |
| Example 564^{b)} | 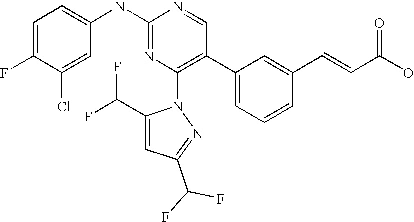 (2E)-3-(3-{4-[3,5- bis(difluoromethyl)-1H- pyrazol-1-yl]-2-[(3-chloro- 4- fluorophenyl)amino] pyrimidin-5-yl}phenyl)prop-2- enoic acid | MS(ES): 536 (M + 1) for C₂₄H₁₅ClF₅N₅O₂. 400 MHz, DMSO-d₆: δ 6.47 (d, J = 15.84 Hz, 1H), 6.86 (t, J = 53.76 Hz, 1H), 6.97 (m, 1H), 7.23 (br s, 1H), 7.43 (m, 5H), 7.62 (br s, 2H), 8.05 (br s, 1H), 8.95 (s, 1H), 10.40 (br s, 1H), 12.44 (br s, 1H). | Example 556 ethyl (2E)-3- (3-{4-[3,5- bis(difluoro- methyl)-1H- pyrazol-1-yl]- 2-[(3-chloro- 4- fluorophenyl) amino] pyrimidin-5- yl}phenyl)prop- 2-enoate |
| Example 565^{c)} | 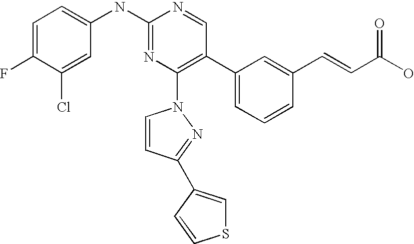 (2E)-3-(3-{2-[(3-chloro-4- fluorophenyl)amino]-4-[3- (thiophen-3-yl)-1H- pyrazol-1-yl]pyrimidin-5- yl}phenyl)prop-2-enoic acid | MS(ES): 518 (M + 1) for C₂₆H₁₇ClFN₅O₂S. 400 MHz, DMSO-d₆: δ 6.52 (d, J = 16.00 Hz, 1H), 6.92 (d, J = 2.40 Hz, 1H), 7.01 (d, J = 4.80 Hz, 1H), 7.26 (d, J = 7.60 Hz, 1H), 7.39-7.45 (m, 2H), 7.51- 7.55 (m, 2H), 7.65-7.68 (m, 3H), 7.72-7.76 (m, 1H), 8.15 (dd, J = 2.40, 6.80 Hz, 1H), 8.32 (d, J = 2.80 Hz, 1H), 8.63 (br s, 1H), 10.3 (br s, 1H). | Example 557 ethyl (2E)-3- (3-{2-[(3- chloro-4- fluorophenyl) amino]-4-[3- (thiophen-3- yl)-1H- pyrazol-1- yl]pyrimidin- 5- yl}phenyl) prop-2-enoate |
| Example 566^{a)} | 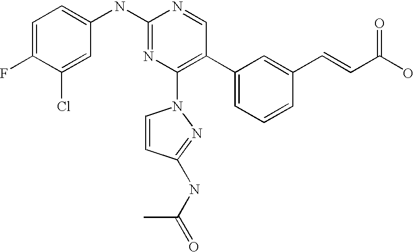 (2E)-3-(3-{4-[3- (acetylamino)-1H-pyrazol- 1-yl]-2-[(3-chloro-4- fluorophenyl)amino] pyrimidin-5-yl}phenyl)prop-2- enoic acid | MS(ES): 493 (M + 1) for C₂₄H₁₈ClFN₆O₃. 400 MHz, DMSO-d₆: δ 1.95 (s, 3H), 6.53 (d, J = 15.60 Hz, 1H), 6.83 (d, J = 2.40 Hz, 1H), 7.06 (d, J = 7.20 Hz, 1H), 7.34 (t, J = 7.60 Hz, 1H), 7.40 (t, J = 9.20 Hz, 1H), 7.45 (d, J = 16.00 Hz, 1H), 7.56 (s, 1H), 7.58 (br s, 1H), 7.73- 7.76 (m, 1H), 8.08 (dd, J = 2.40, 6.80 Hz, 1H), 8.13 (d, J = 2.40 Hz, 1H), 8.68 (br s, 1H), 10.24 (s, 1H), 10.43 (s, 1H). | Example 558 ethyl (2E)-3- (3-{4-[3- (acetylamino) 1H-pyrazol- 1-yl]-2-[(3- chloro-4- fluorophenyl) amino] pyrimidin-5- yl}phenyl)prop- 2-enoate |
| Example 567^{a)} | 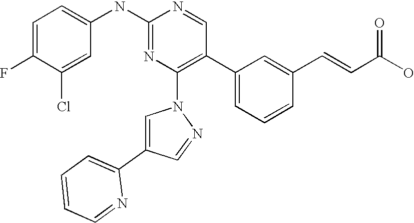 (2E)-3-(3-{2-[(3-chloro-4- fluorophenyl)amino]-4-[4- (pyridin-2-yl)-1H-pyrazol- 1-yl]pyrimidin-5- yl}phenyl)prop-2-enoic acid | MS(ES): 511 (M − 1) for C₂₇H₁₈ClFN₆O₂. 400 MHz, DMSO-d₆: δ 6.52 (d, J = 16.00 Hz, 1H), 7.20 (d, J = 7.44 Hz, 1H), 7.25 (t, J = 6.20 Hz, 1H), 7.37 (t, J = 7.64 Hz, 1H), 7.41 (t, J = 9.28 Hz, 1H), 7.57 (d, J = 15.96 Hz, 1H), 7.62 (d, J = 7.64 Hz, 1H), 7.65 (s, 1H), 7.69-7.76 (m, 2H), 7.82 (t, J = 7.60 Hz, 1H), 8.15 (dd, J = 2.48, 6.72 Hz, 1H), 8.18 (s, 1H), 8.56 (d, J = 4.20 Hz, 1H), 8.71 (s, 1H), 8.90 (s, 1H), 10.31 (s, 1H), 12.40 (br s, 1H). | Example 559 ethyl (2E)-3- (3-{2-[(3- chloro-4- fluorophenyl) amino]-4-[4- (pyridin-2- yl)-1H- pyrazol-1- yl]pyrimidin- 5- yl}phenyl) prop-2-enoate |
| Example 568^{a)} | 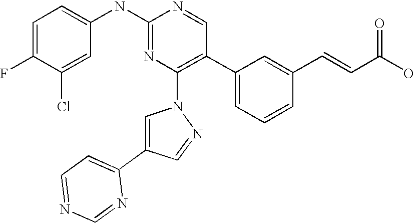 (2E)-3-(3-{2-[(3-chloro-4- fluorophenyl)amino]-4-[4- (pyrimidin-4-yl)-1H- pyrazol-1-yl]pyrimidin-5- yl}phenyl)prop-2-enoic acid. | MS(ES): 512 (M − 1) for C₂₆H₁₇ClFN₇O₂. 400 MHz, DMSO-d₆: δ 6.51 (d, J = 16.00 Hz, 1H), 7.19 (d, J = 7.76 Hz, 1H), 7.36 (t, J = 7.64 Hz, 1H), 7.41 (t, J = 9.12 Hz, 1H), 7.56 (d, J = 16.00 Hz, 1H), 7.61 (s, 1H), 7.64 (br s, 1H), 7.70-7.74 (m, 1H), 7.85-7.86 (m, 1H), 8.13 (dd, J = 2.56, 6.76 Hz, 1H), 8.30 (s, 1H), 8.75 (s, 1H), 8.78 (d, J = 5.32 Hz, 1H), 9.08 (s, 1H), 9.12 (s, 1H), 10.35 (s, 1H), 12.38 (br s, 1H). | Example 560 ethyl (2E)-3- (3-{2-[(3- chloro-4- fluorophenyl) amino]-4-[4- (pyrimidin-4- yl)-1H- pyrazol-1- yl]pyrimidin- 5- yl}phenyl) prop-2-enoate. |

^{a)}Method L
^{b)}Method M
^{c)}Method M, ambient temperature

### Example 569

### methyl 3-(2-(3-chloro-4-fluorophenylamino)-4-(3-(trifluoromethyl)-1H-pyrazol-1-yl)pyrimidin-5-yl)benzoate

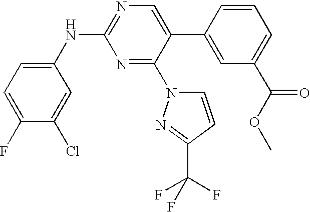

The title compound was prepared using the general method described above for Example 1 using 3-(methoxycarbonyl)phenylboronic acid and intermediate 115 as starting materials.

MS: ES+ 492 for C₂₂H₁₄ClF₄N₅O₂.

1H NMR (300 MHz, DMSO-D6) δ ppm 3.82 (s, 3H), 6.99 (d, 1H), 7.35-7.55 (m, 3H), 7.64-7.76 (m, 2H), 7.91 (d, 1H), 8.09 (dd, 1H), 8.40 (s, 1H), 8.80 (s, 1H), 10.43 (s, 1H).

### Example 570

### 3-(2-(3-chloro-4-fluorophenylamino)-4-(3-(trifluoromethyl)-1H-pyrazol-1-yl)pyrimidin-5-yl)benzoic acid

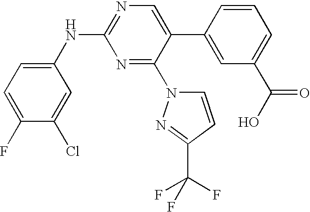

The title compound was prepared using the general method described above for Example 214 using 1N sodium hydroxide (2 equivalents), dioxane:THF (1:1) as solvent and (Example 569 as a starting material.

MS: ES+ 478 for C₂₁H₁₂ClF₄N₅O₂.

1H NMR (400 MHz, DMSO-D6) δ ppm 6.98 (d, 1H), 7.37-7.51 (m, 3H), 7.66 (s, 1H), 7.69-7.76 (m, 1H), 7.88 (d, 1H), 8.10 (dd, 1H), 8.39 (s, 1H), 8.79 (s, 1H), 10.40 (s, 1H), 12.93 (s, 1H).

### Example 571

### (1,3-trans)-3-(3-(2-(4-fluorophenylamino)-4-(3-(trifluoromethyl)-1H-pyrazol-1-yl)pyrimidin-5-yl)phenyl)-2,2-dimethylcyclopropanecarboxylic acid

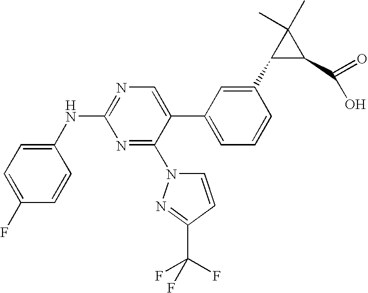

(1,3-trans)-tert-butyl 2,2-dimethyl-3-(3-(4,4,5,5-tetramethyl-1,3,2-dioxaborolan-2-yl)phenyl)cyclopropanecarboxylate Intermediate 174 (342 mg, 0.92 mmol), 5-bromo-N-(3-chloro-4-fluorophenyl)-4-(3-(trifluoromethyl)-1H-pyrazol-1-yl)pyrimidin-2-amine (200 mg, 0.46 mmol) (Intermediate 115), Tris(dibenzylideneacetone)dipalladium(0) (41.9 mg, 0.05 mmol), 2-Dicyclohexylphosphino-2′,4′,6′-triisopropylbiphenyl (65.5 mg, 0.14 mmol), Na₂CO₃ (200 mg, 1.89 mmol), acetonitrile (3 mL) and water (0.750 mL) were combined and degassed with an argon stream for 10 min. The mixture was warmed at 80° C. for 2.5 hrs, allowed to cool, diluted with acetonitrile, filtered and adsorbed on 15 ml silica gel. Purified by flash chromatography (80 g cartridge, 0-10% ethyl acetate in hexane). A crude sample of the tert-butyl ester of the title compound was thus obtained as a yellow-orange solid: (100 mg). This sample was converted to the title compound as follows: Sample was first combined with dichloromethane (1 mL) and trifluoroacetic acid (1 mL, 12.98 mmol). The clear solution was stirred at room temperature for 45 min then evaporated. The mixture was dissolved in dichloromethane and applied to 5 ml silica gel column. Eluted with 20% ethyl acetate in hexane, then with 50% ethyl acetate in hexane. Pure fractions evaporated and dissolved in 0.25 ml ethyl acetate then precipitated with 5 ml hexanes. Filtered to give a white solid (9 mg).

MS: ES+ 512 for C₂₆H₂₁F₄N₅O₂.

1H NMR (400 MHz, DMSO-D6) δ ppm 0.91 (s, 3H), 1.32 (s, 3H), 2.05 (d, 1H), 2.56 (d, 1H), 7.14 (d, 1H), 7.24-7.28 (m, 1H), 7.31 (d, 2H), 7.37 (s, 1H), 7.40-7.44 (m, 2H), 7.80-7.87 (m, 2H), 8.64 (d, 1H), 8.72 (d, 1H), 10.06 (s, 1H), 12.20 (s, 1H).

### Example 572

### (1,3-trans)-3-(3-(2-(3-chloro-4-fluorophenylamino)-4-(3-(trifluoromethyl)-1H-pyrazol-1-yl)pyrimidin-5-yl)phenyl)-2,2-dimethylcyclopropanecarboxylic acid

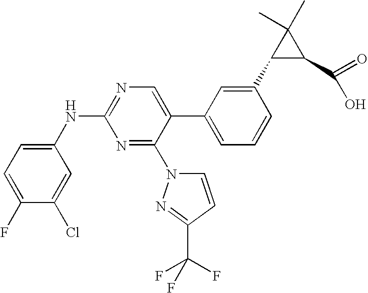

(1S,3S)-tert-butyl 3-(3-(2-(3-chloro-4-fluorophenylamino)-4-(3-(trifluoromethyl)-1H-pyrazol-1-yl)pyrimidin-5-yl)phenyl)-2,2-dimethylcyclopropanecarboxylate Intermediate 176 (35 mg, 0.06 mmol) was dissolved in dichloromethane (1 mL) then trifluoroacetic acid (1 ml, 12.98 mmol) was added to give a clear yellow solution. The mixture was stirred at room temperature for 30 minutes. The solution was concentrated and the residue was triturated with 3 ml hexane to give the title compound as a light yellow solid (30 mg).

MS: ES+ 546 for C₂₆H₂₀ClF₄N₅O₂.

1H NMR (400 MHz, DMSO-D6) δ ppm 0.77 (s, 3H), 1.26 (s, 3H), 1.87 (d, 1H), 2.40 (d, 1H), 6.90 (s, 1H), 6.93 (d, 1H), 7.06 (d, 1H), 7.17 (d,1H), 7.29 (t, 1H), 7.39 (t, 1H), 7.66-7.73 (m, 1H), 8.12 (dd, 1H), 8.24 (s, 1H), 8.77 (s, 1H), 10.38 (s, 1H).

### Example 573

### (1,2-trans)-2-(5-(2-(3-chloro-4-fluorophenvlamino)-4-(3-(trifluoromethyl)-1H-pyrazol-1-yl)pyrimidin-5-yl)pyridin-3-yl)cyclopropanecarboxylic acid

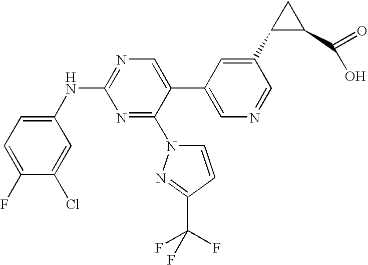

The title compound was prepared using the general method described above for Example 572 using Intermediate 180 as a starting material.

MS: ES+ 519 for C23H15ClF4N6O2.

1H NMR (300 MHz, DMSO-D6) δ ppm 0.78-0.90 (m, 1H), 1.28-1.38 (m, 1H), 1.40-1.51 (m, 1H), 1.81-1.93 (m, 1H), 7.05 (d, 1H), 7.42 (t,1H), 7.48 (m, 1H), 7.67-7.76 (m, 1H), 8.08 (dd, 1H), 8.31 (s, 1H), 8.49 (s, 1H), 8.54 (s, 1H), 8.82 (s, 1H), 10.47 (s, 1H).

### Example 574

### (1,2-trans)-2-(3-(2-(3-chloro-4-fluorophenylamino)-4-(3-(trifluoromethyl)-1H-pyrazol-1-yl)pyrimidin-5-yl)phenyl)cyclopropanecarboxylic acid

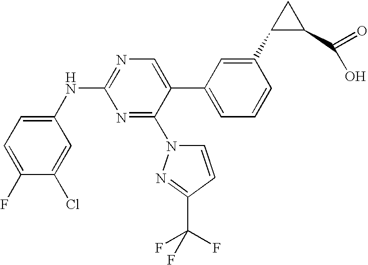

The title compound was prepared using the general method described above for Example 572 using Intermediate 183 as a starting material.

MS: ES+ 518 for C24H16ClF4N5O2.

1H NMR (400 MHz, DMSO-D6) δ ppm 1.17-1.27 (m, 1H), 1.33-1.41 (m, 1H), 1.68-1.76 (m, 1H), 2.28-2.37 (m, 1H), 6.86 (s, 1H), 6.94-7.00 (m, 2H), 7.16 (d, 1H), 7.24 (t, 1H), 7.39 (t, 1H), 7.66-7.73 (m, 1H), 8.12 (dd, 1H), 8.24 (s, 1H), 8.79 (s, 1H), 10.38 (s, 1H), 12.25 (s, 1H).

### Examples 575 and 576

### (1R,2R)-2-(3-(2-(3-chloro-4-fluorophenvlamino)-4-(3-(trifluoromethyl)-1H-pyrazol-1-yl)pyrimidin-5-yl)phenyl)cyclopropanecarboxylic acid and (1S,2R)-2-(3-(2-(3-chloro-4-fluorophenvlamino)-4-(3-(trifluoromethyl)-1H-pyrazol-1-yl)pyrimidin-5-yl)phenyl)cyclopropanecarboxylic acid

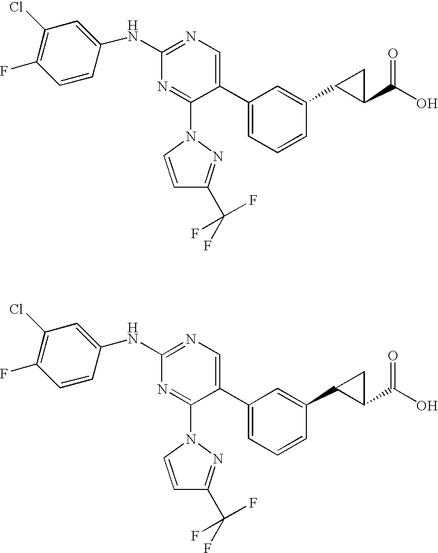

Racemic (1,2-trans)-tert-butyl 2-(3-(2-(3-chloro-4-fluorophenylamino)-4-(3-(trifluoromethyl)-1H-pyrazol-1-yl)pyrimidin-5-yl)phenyl)cyclopropanecarboxylate Intermediate 183 (170 mg) was subjected to preparative chiral supercritical fluid chromatography using an instrument manufactured by Berger. Column: Chiralpak AD-H, dimensions: 250×21mm, 5μ; modifier: 25% isopropanol with 0.4% dimethylethylamine; flow rate: 60 ml/min; oven temperature: 40° C., outlet pressure: 100 bar. Samples of the separate ester enantiomers were thus obtained in >98% e.e. (60 mg each). These samples were separately deprotected under the conditions described for the racemate Example 574 to give the enantiopure title compounds. The absolute stereochemistry of the samples was unassigned, Example 575 displays a (+) rotation and Example 576 displays a (−) rotation. Mass spectral and 1H NMR data for both enantiomers are identical to that of the racemic sample.

### Example 577

### methyl 5-(2-(3,5-dimethoxyphenylamino)-4-(3-(trifluoromethyl)-1H-pyrazol-1-yl)pyrimidin-5-yl)-2-(2-(methylsulfonyflethylamino)nicotinate

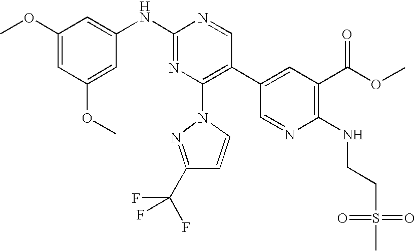

Methyl 5-bromo-2-(2-(methylsulfonyl)ethylamino)nicotinate Intermediate 188 (200 mg, 0.59 mmol), PdCl2 (dppf)-CH₂Cl₂ adduct (72.7 mg, 0.09 mmol), bis(pinacolato)diboron (181 mg, 0.71 mmol) and potassium acetate (175 mg, 1.78 mmol) were combined in dioxane (4 mL). Argon was bubbled through the mixture for 10 minutes and then it was warmed at 90° C. for 18 hours. The dark suspension was filtered through a celite pad and the solids were rinsed thoroughly with dichloromethane. The filtrate was concentrated to give the crude boronate ester intermediate: methyl 2-(2-(methylsulfonyl)ethylamino)-5-(4,4,5,5-tetramethyl-1,3,2-dioxaborolan-2-yl)nicotinate (420 mg). This material was combined with 5-bromo-N-(3,5-dimethoxyphenyl)-4-(3-(trifluoromethyl)-1H-pyrazol-1-yl)pyrimidin-2-amine Intermediate 215 (133 mg, 0.30 mmol), Tris(dibenzylideneacetone)dipalladium(0) (27.4 mg, 0.03 mmol), Sodium carbonate (95 mg, 0.90 mmol) and 2-Dicyclohexylphosphino-2′,4′,6′-triisopropylbiphenyl (42.8 mg, 0.09 mmol) then suspended in acetonitrile (3 mL) and water (0.75 mL). The mixture was degassed with argon stream for 10 min then heated at 80° C. for 1 hour. The mixture was diluted with acetonitrile and adsorbed on 10 ml silica gel. Flash chromatography (0 to 50% acetonitrile in dichloromethane, 25 g cartridge) gave the title compound as a yellow solid (133 mg).

MS: ES+ 662 for C₂₆H₂₆F₃N₇O₆S.

1H NMR (400 MHz, DMSO-d6) δ ppm 3.02 (s, 3H), 3.41 (t, 2H), 3.74 (s, 6H), 3.77 (s, 3H), 3.89-3.99 (m, 2H), 6.20 (t, 1H), 7.02 (d, 1H),7.10 (d, 2H), 7.80 (d, 1H), 8.19 (t, 1H), 8.22 (d, 1H), 8.43 (d, 1H), 8.77 (s, 1H), 10.12 (s, 1H)

The compounds in the table below were prepared using the general procedure described above for Example 577 and the starting materials listed.

| | | Mass spectrum and | |
|---|---|---|---|
| Compound | Structure | ¹H NMR | SM |
| Example 578 | 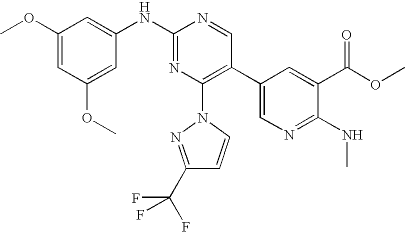 methyl 5-(2-(3,5- dimethoxyphenylamino)- 4-(3-(trifluoromethyl)- 1H-pyrazol-1- yl)pyrimidin-5-yl)-2- (methylamino)nicotinate | MS: ES+ 530 for C₂₄H₂₂F₃N₇O₄ 1H NMR (400 MHz, DMSO-d6) δ ppm 2.97 (d, 3H), 3.74 (s, 6H), 3.76 (s, 3H), 6.19 (t, 1H), 7.01 (d, 1H), 7.10 (d, 2H), 7.74 (d, 1H), 7.89 (q, 1H), 8.19 (d, 1H), 8.40 (d, 1H), 8.77 (s, 1H), 10.11 (s, 1H) | Intermediate 184 methyl 5-bromo-2- (methylamino) nicotinate And Intermediate 215 5-bromo-N-(3,5- dimethoxyphenyl)-4- (3-(trifluoromethyl)- 1H-pyrazol-1- yl)pyrimidin-2-amine |
| Example 579 | 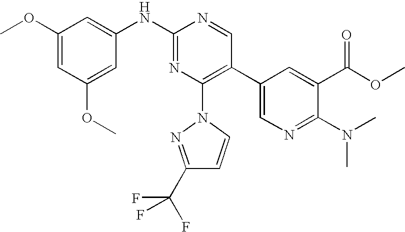 methyl 5-(2-(3,5- dimethoxyphenylamino)- 4-(3-(trifluoromethyl)- 1H-pyrazol-1- yl)pyrimidin-5-yl)-2- (dimethylamino) nicotinate | MS: ES+ 544 for C₂₅H₂₄F₃N₇O₄ H NMR (400 MHz, DMSO-d6) δ ppm 2.95 (s, 6H), 3.74 (s, 6H), 3.76 (s, 3H), 6.19 (s, 1H), 7.01 (d, 1H), 7.10 (d, 2H), 7.54 (d, 1H), 8.09 (d, 1H), 8.41 (d, 1H), 8.77 (s, 1H), 10.11 (s, 1H) | Intermediate 185 methyl 5-bromo-2- (dimethylamino) nicotinate And Intermediate 215 5-bromo-N-(3,5- dimethoxyphenyl)-4- (3-(trifluoromethyl)- 1H-pyrazol-1- yl)pyrimidin-2-amine |
| Example 580 | 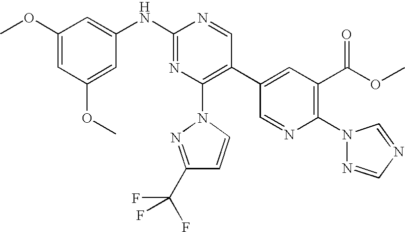 methyl 5-(2-(3,5- dimethoxyphenylamino)- 4-(3-(trifluoromethyl)- 1H-pyrazol-1- yl)pyrimidin-5-yl)-2- (1H-1,2,4-triazol-1- yl)nicotinate | MS: ES+ 568 for C₂₅H₂₀F₃N₉O₄ 1H NMR (400 MHz, DMSO-d6) δ ppm 3.73 (s, 3H), 3.76 (s, 6H), 6.24 (t, 1H), 7.08 (d, 1H), 7.10 (d, 2H), 8.10 (d, 1H), 8.29 (s, 1H), 8.57 (d, 1H), 8.58 (d, 1H), 8.85 (s, 1H), 9.32 (s, 1H), 10.27 (s, 1H) | Intermediate 186 methyl 5-bromo-2- (1H-1,2,4-triazol-1- yl)nicotinate And Intermediate 215 5-bromo-N-(3,5- dimethoxyphenyl)-4- (3-(trifluoromethyl)- 1H-pyrazol-1- yl)pyrimidin-2-amine |
| Example 581 | 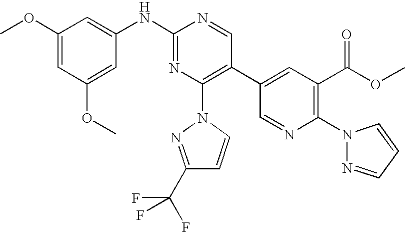 methyl 5-(2-(3,5- dimethoxyphenylamino)- 4-(3-(trifluoromethyl)- 1H-pyrazol-1- yl)pyrimidin-5-yl)-2- (1H-pyrazol-1- yl)nicotinate | MS: ES+ 567 for C₂₆H₂₁F₃N₈O₄ 1H NMR (400 MHz, DMSO-d6) δ ppm 3.71 (s, 3H), 3.75 (s, 6H), 6.17-6.26 (m, 1H), 6.59 (s, 1H), 7.06 (d, 1H), 7.10 (d, 2H), 7.80 (s, 1H), 7.92 (d, 1H), 8.45 (d, 1H), 8.53 (d, 2H), 8.83 (s, 1H), 10.23 (s, 1H) | Intermediate 187 methyl 5-bromo-2- (1H-pyrazol-1- yl)nicotinate And Intermediate 215 5-bromo-N-(3,5- dimethoxyphenyl)-4- (3-(trifluoromethyl)- 1H-pyrazol-1- yl)pyrimidin-2-amine |
| Example 582 | 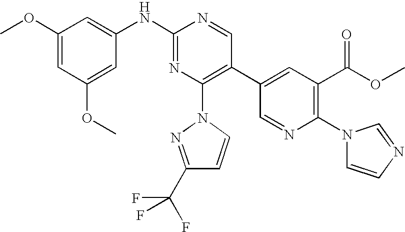 methyl 5-(2-(3,5- dimethoxyphenylamino)- 4-(3-(trifluoromethyl)- 1H-pyrazol-1- yl)pyrimidin-5-yl)-2- (1H-imidazol-1- yl)nicotinate | MS: ES+ 567 for C₂₆H₂₁F₃N₈O₄ 1H NMR (400 MHz, DMSO-d6) δ ppm 3.75 (s, 9H), 6.23 (t, 1H), 7.03-7.14 (m, 4H), 7.50 (s, 1H), 8.04 (s, 1H), 8.18 (d, 1H), 8.56 (d, 1H), 8.59 (d, 1H), 8.84 (s, 1H), 10.25 (s, 1H) | Intermediate 189 methyl 5-bromo-2- (1H-imidazol-1- yl)nicotinate And Intermediate 215 5-bromo-N-(3,5- dimethoxyphenyl)-4- (3-(trifluoromethyl)- 1H-pyrazol-1- yl)pyrimidin-2-amine |
| Example 583 | 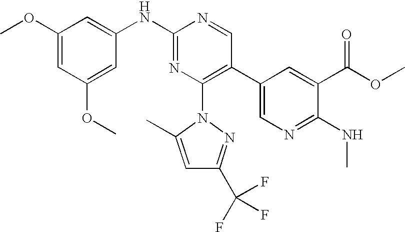 methyl 5-(2-(3,5- dimethoxyphenylamino)- 4-(5-methyl-3- (trifluoromethyl)-1H- pyrazol-1-yl)pyrimidin- 5-yl)-2- (methylamino)nicotinate | MS: ES+ 544 for C₂₅H₂₄F₃N₇O₄ 1H NMR (400 MHz, DMSO-d6) δ ppm 2.15 (s, 3H), 2.95 (d, 3H), 3.72 (s, 6H), 3.74 (s, 3H), 6.19 (t, 1H), 6.73 (s, 1H), 7.05 (d, 2H), 7.46 (d, 1H), 7.83- 7.95 (m, 1H), 8.22 (d, 1H), 8.93 (s, 1H), 10.15 (s, 1H) | Intermediate 184 methyl 5-bromo-2- (methylamino) nicotinate And Intermediate 216 5-bromo-N-(3,5- dimethoxyphenyl)-4- (5-methyl-3- (trifluoromethyl)-1H- pyrazol-1- yl)pyrimidin-2-amine |
| Example 584 | 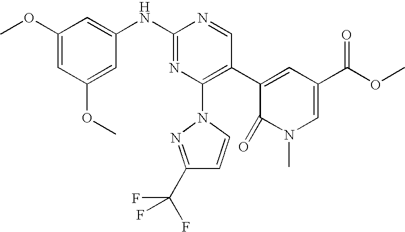 methyl 5-(2-(3,5- dimethoxyphenylamino)- 4-(3-(trifluoromethyl)- 1H-pyrazol-1- yl)pyrimidin-5-yl)-1- methyl-6-oxo-1,6- dihydropyridine-3- carboxylate | MS: ES+ 531 for C24H21F3N6O5 1H NMR (400 MHz, DMSO-d6) δ ppm 3.40 (s, 3H), 3.75 (s, 6H), 3.82 (s, 3H), 6.21 (t, 1H), 7.00 (d, 1H), 7.08 (d, 2H), 7.90 (d, 1H), 8.59 (d, 1H), 8.61 (d, 1H), 8.64 (s, 1H), 10.09 (s, 1H) | Intermediate 190 methyl 5-bromo-1- methyl-6-oxo-1,6- dihydropyridine-3- carboxylate And Intermediate 215 5-bromo-N-(3,5- dimethoxyphenyl)-4- (3-(trifluoromethyl)- 1H-pyrazol-1- yl)pyrimidin-2-amine |

The compounds in the below table were prepared using the general method described above for Example 214 using 1N sodium hydroxide (2 equivalents), dioxane:THF (1:1) as solvent and the starting material indicated.

| | | Mass spectrum and ¹H | |
|---|---|---|---|
| Compound | Structure | NMR | SM |
| Example 585 | 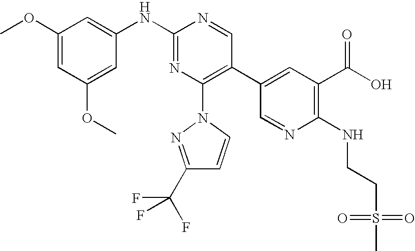 5-(2-(3,5- dimethoxyphenylamino)- 4-(3-(trifluoromethyl)- 1H-pyrazol-1- yl)pyrimidin-5-yl)-2-(2- (methylsulfonyl)ethyl- amino)nicotinic acid | MS: ES+ 608 for C₂₅H₂₄F₃N₇O₆S 1H NMR (400 MHz, DMSO-d6) δ ppm 3.01 (s, 3H), 3.41 (t, 2H), 3.73 (s, 6H), 3.92 (dd, 2H), 6.19 (t, 1H), 7.01 (d, 1H), 7.10 (d, 2H), 7.78 (d, 1H), 8.19 (d, 1H), 8.29-8.39 (m, 1H), 8.40- 8.46 (m, 1H), 8.76 (s, 1H), 10.11 (s, 1H), 13.09 (bs, 1H). | Example 577 methyl 5-(2-(3,5- dimethoxyphenyl- amino)-4-(3- (trifluoromethyl)- 1H-pyrazol-1- yl)pyrimidin-5-yl)- 2-(2- (methylsulfonyl) ethylamino)nico- tinate |
| Example 586 | 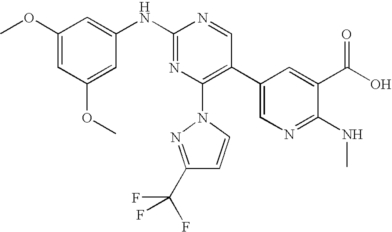 5-(2-(3,5- dimethoxyphenylamino)- 4-(3-(trifluoromethyl)- 1H-pyrazol-1- yl)pyrimidin-5-yl)-2- (methylamino)nicotinic acid | MS: ES+ 516 for C₂₃H₂₀F₃N₇O₄ H NMR (400 MHz, DMSO-d6) δ ppm 2.99 (s, 3H), 3.73 (s, 6H), 6.19 (t, 1H), 7.01 (d, 1H), 7.10 (d, 2H), 7.79 (br. s., 1H), 8.17 (d, 1H), 8.41 (s, 1H), 8.75 (s, 1H), 10.11 (s, 1H), 13.07 (br. s., 1H) | Example 578 methyl 5-(2-(3,5- dimethoxyphenyl- amino)-4-(3- (trifluoromethyl)- 1H-pyrazol-1- yl)pyrimidin-5-yl)- 2- (methylamino) nicotinate |
| Example 587 | 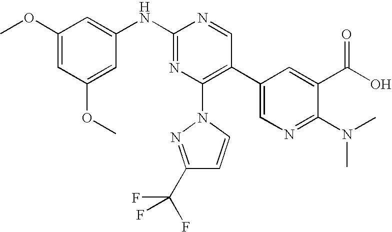 5-(2-(3,5- dimethoxyphenylamino)- 4-(3-(trifluoromethyl)- 1H-pyrazol-1- yl)pyrimidin-5-yl)-2- (dimethylamino)nicotinic acid | MS: ES+ 531 for C₂₄H₂₂F₃N₇O₄ 1H NMR (400 MHz, DMSO-d6) δ ppm 2.96 (s, 6H), 3.74 (s, 6H), 6.19 (t, 1H), 6.95-7.05 (m, 1H), 7.06-7.14 (m, 2H), 7.54 (d, 1H), 8.02 (d, 1H), 8.37 (s, 1H), 8.74-8.79 (m, 1H), 10.10 (s, 1H), 12.84 (br. s., 1H) | Example 579 methyl 5-(2-(3,5- dimethoxyphenyl- amino)-4-(3- (trifluoromethyl)- 1H-pyrazol-1- yl)pyrimidin-5-yl)- 2- (dimethylamino)- nicotinate |
| Example 588 | 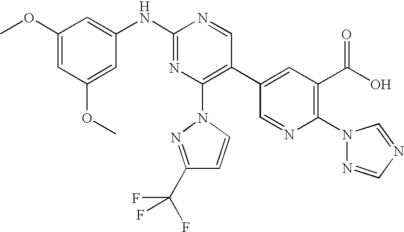 5-(2-(3,5- dimethoxyphenylamino)- 4-(3-(trifluoromethyl)- 1H-pyrazol-1- yl)pyrimidin-5-yl)-2- (1H-1,2,4-triazol-1- yl)nicotinic acid | MS: ES+ 554 for C₂₄H₁₈F₃N₉O₄ 1H NMR (400 MHz, DMSO-d6) δ ppm 2.96 (s, 6H), 3.74 (s, 6H), 6.19 (t, 1H), 6.95-7.05 (m, 1H), 7.06-7.14 (m, 2H), 7.54 (d, 1H), 8.02 (d, 1H), 8.37 (s, 1H), 8.74-8.79 (m, 1H), 10.10 (s, 1H), 12.84 (br. s., 1H) | Example 580 methyl 5-(2-(3,5- dimethoxyphenyl- amino)-4-(3- (trifluoromethyl)- 1H-pyrazol-1- yl)pyrimidin-5-yl)- 2-(1H-1,2,4-triazol- 1-yl)nicotinate |
| Example 589 | 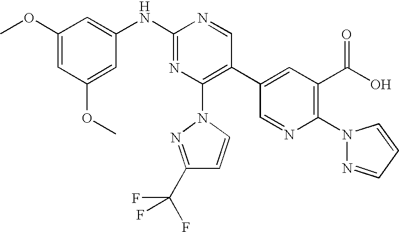 5-(2-(3,5- dimethoxyphenylamino)- 4-(3-(trifluoromethyl)- 1H-pyrazol-1- yl)pyrimidin-5-yl)-2- (1H-pyrazol-1- yl)nicotinic acid | MS: ES+ 553 for C25H19F3N8O4 1H NMR (400 MHz, DMSO-d6) δ ppm 3.75 (s, 6H), 6.23 (t, 1H), 6.57 (t, 1H), 7.05 (d, 1H), 7.10 (d, 2H), 7.75- 7.80 (m, 1H), 7.89 (d, 1H), 8.40 (d, 1H), 8.47 (d, 1H), 8.50-8.56 (m, 1H), 8.84 (s, 1H), 10.23 (s, 1H), 13.12 (s, 1H) | Example 581 methyl 5-(2-(3,5- dimethoxyphenyl- amino)-4-(3- (trifluoromethyl)- 1H-pyrazol-1- yl)pyrimidin-5-yl)- 2-(1H-pyrazol-1- yl)nicotinate |
| Example 590 | 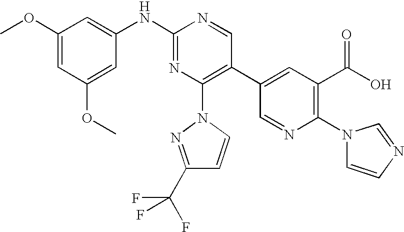 5-(2-(3,5- dimethoxyphenylamino)- 4-(3-(trifluoromethyl)- 1H-pyrazol-1- yl)pyrimidin-5-yl)-2- (1H-imidazol-1- yl)nicotinic acid | MS: ES+ 553 for C25H19F3N8O4 1H NMR (400 MHz, DMSO-d6) δ ppm 3.76 (s, 6H), 6.24 (t, 1H), 7.07 (d, 1H), 7.10 (d, 2H), 7.38 (s, 1H), 7.73 (s, 1H), 8.23 (d, 1H), 8.56 (d, 1H), 8.62 (d, 2H), 8.85 (s, 1H), 10.26 (s, 1H), 13.65 (br. s., 1H) | Example 582 methyl 5-(2-(3,5- dimethoxyphenyl- amino)-4-(3- (trifluoromethyl)- 1H-pyrazol-1- yl)pyrimidin-5-yl)- 2-(1H-imidazol-1- yl)nicotinate |
| Example 591 | 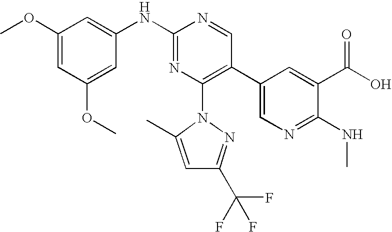 5-(2-(3,5- dimethoxyphenylamino)- 4-(5-methyl-3- (trifluoromethyl)-1H- pyrazol-1-yl)pyrimidin- 5-yl)-2- (methylamino)nicotinic acid | MS: ES+ 530 for C₂₄H₂₂F₃N₇O₄ 1H NMR (400 MHz, DMSO-d6) δ ppm 2.21 (s, 3H), 2.95 (s, 3H), 3.72 (s, 6H), 6.19 (s, 1H), 6.72 (s, 1H), 7.05 (d, 2H), 7.55 (d, 1H), 8.06- 8.18 (m, 2H), 8.90 (s, 1H), 10.13 (s, 1H), 13.05 (br. s., 1H) | Example 583 methyl 5-(2-(3,5- dimethoxyphenyl- amino)-4-(5-methyl- 3-(trifluoromethyl)- 1H-pyrazol-1- yl)pyrimidin-5-yl)- 2-(methylamino)- nicotinate |
| Example 592 | 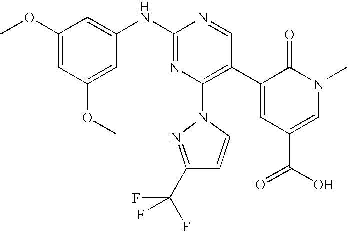 5-(2-(3,5- dimethoxyphenylamino)- 4-(3-(trifluoromethyl)- 1H-pyrazol-1- yl)pyrimidin-5-yl)-1- methyl-6-oxo-1,6- dihydropyridine-3- carboxylic acid | MS: ES+ 517 for C₂₃H₁₉F₃N₆O₅ 1H NMR (400 MHz, DMSO-d6) δ ppm 3.39 (s, 3H), 3.75 (s, 6H), 6.21 (s, 1H), 7.00 (d, 1H), 7.08 (d, 2H), 7.87 (d, 1H), 8.53 (d, 1H), 8.56- 8.60 (m, 1H), 8.64 (s, 1H), 10.08 (s, 1H), 12.86 (br. s., 1H) | Example 584 methyl 5-(2-(3,5- dimethoxyphenyl- amino)-4-(3- (trifluoromethyl)- 1H-pyrazol-1- yl)pyrimidin-5-yl)- 1-methyl-6-oxo- 1,6- dihydropyridine-3- carboxylate |

### General Method for Biaryl Synthesis

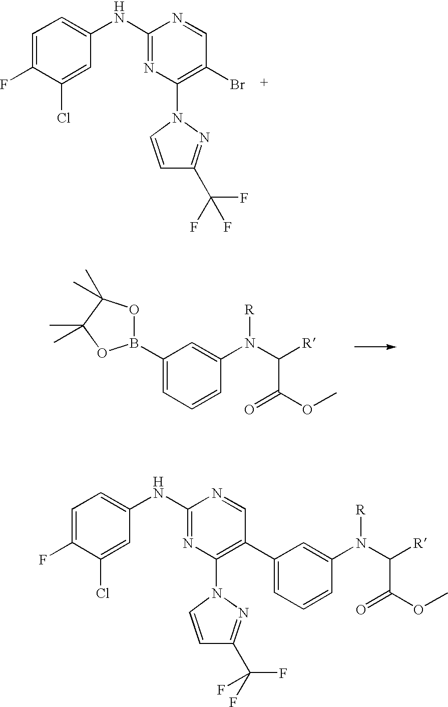

A suspension of 5-bromo-N-(3-chloro-4-fluorophenyl)-4-[3-(trifluoromethyl)-1H-pyrazol-1-yl]pyrimidin-2-amine (Intermediate 115, 1 eq), boronate derivative (1.1 eq), [1,1′-bis(diphenylphosphino)ferrocene]dichloropalladium(II) (20 mol %) and sodium carbonate (1 eq) in acetonitrile/water (4:1) was degassed and heated to 90° C. for 15-20 min under inert atmosphere. The solvent was removed in vacuo and the crude mixture taken in CHCl₃ was washed with water, brine, dried over Na₂SO₄, filtered and concentrated. The crude material was purified by silica gel column chromatography using chloroform:methanol (9:1) as eluent to give the product. The compounds in the below table were prepared using this general procedure and the starting material specified.

| Compound | Structure | Mass spectrum and ¹H NMR | SM |
|---|---|---|---|
| Example 593 | 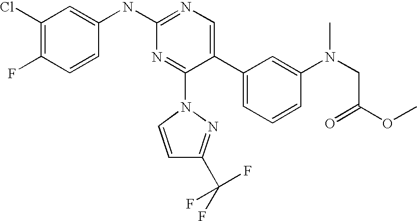 methyl N-(3-{2-[(3-chloro-4- fluorophenyl)amino]-4-[3- (trifluoromethyl)-1H- pyrazol-1-yl]pyrimidin-5- yl}phenyl)-N- methylglycinate | The compound was taken to the next step on the basis of LCMS. MS(ES): 535 (M + 1) for C₂₄H₁₉ClF₄N₆O₂. (91% pure by LCMS) | Intermediate 201 methyl N- methyl-N- [3- (4,4,5,5- tetramethyl- 1,3,2- dioxaborolan- 2- yl)phenyl] glycinate |
| Example 594 | 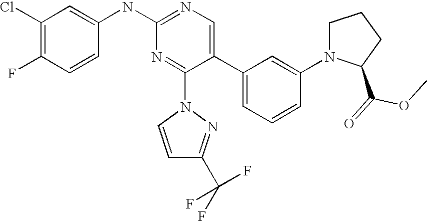 methyl 1-(3-{2-[(3-chloro- 4-fluorophenyl)amino]-4- [3-(trifluoromethyl)-1H- pyrazol-1-yl]pyrimidin-5- yl}phenyl)-L-prolinate | MS(ES): 561 (M + 1) for C₂₆H₂₁ClF₄N₆O₂. 400 MHz, DMSO-d₆: δ 2.89 (s, 3H), 3.60 (s, 3H), 4.16 (s, 2H), 6.45 (t, J = 6.40 Hz, 2H), 6.63 (dd, J = 1.60, 8.20 Hz, 1H), 6.95 (d, J = 10.40 Hz, 1H), 7.15 (t, J = 8.00 Hz, 1H), 7.40 (t, J = 9.20 Hz, 1H), 7.67-7.71 (m, 1H), 8.05 (s, 1H), 8.18 (dd, J = 2.40, 6.80 Hz, 1H), 8.79 (s, 1H), 10.41 (s, 1H). | Intermediate 202 methyl 1- [3- (4,4,5,5- tetramethyl- 1,3,2- dioxaborolan- 2- yl)phenyl]-L- prolinate |
| Example 595 | 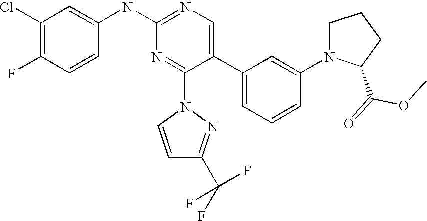 methyl 1-(3-{2-[(3-chloro- 4-fluorophenyl)amino]-4- [3-(trifluoromethyl)-1H- pyrazol-1-yl]pyrimidin-5- yl}phenyl)-D-prolinate | MS(ES): 561 (M + 1) for C₂₆H₂₁ClF₄N₆O₂. The compound was taken to the next step on the basis of LCMS. | Intermediate 203 methyl 1- [3- (4,4,5,5- tetramethyl- 1,3,2- dioxaborolan- 2- yl)phenyl]- D- prolinate |
| Example 596 | 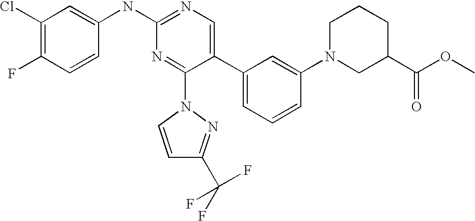 methyl 1-(3-{2-[(3-chloro- 4-fluorophenyl)amino]-4- [3-(trifluoromethyl)-1H- pyrazol-1-yl]pyrimidin-5- yl}phenyl)piperidine-3- carboxylate | MS(ES): 575 (M + 1) for C₂₇H₂₃ClF₄N₆O₂. 300 MHz, DMSO-d₆: δ 1.47- 1.65 (m, 4H), 1.85-1.90 (m, 2H), 2.72 (t, J = 7.53 Hz, 1H), 2.86-2.91 (m, 1H), 3.53-3.55 (m, 1H), 3.61 (s, 3H), 6.58 (s, 1H), 6.33 (d, J = 7.80 Hz, 1H), 6.92-6.94 (m, 2H), 7.19 (t, J = 6.00 Hz, 1H), 7.40 (t, J = 6.84 Hz, 2H), 8.15 (s, 2H), 8.78 (s, 1H), 10.39 (s, 1H). | Intermediate 204 methyl 1- [3- (4,4,5,5- tetramethyl- 1,3,2- dioxaborolan- 2- yl)phenyl] piperidine- 3- carboxylate |
| Example 597 | 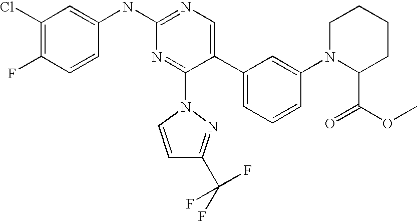 methyl 1-(3-{2-[(3-chloro- 4-fluorophenyl)amino]-4- [3-(trifluoromethyl)-1H- pyrazol-1-yl]pyrimidin-5- yl}phenyl)piperidine-2- carboxylate | Taken to the mext step based on LCMS. MS(ES): 575 (M + 1) for C₂₇H₂₃ClF₄N₆O₂. (96% pure by LCMS) | Intermediate 205 methyl 1- [3- (4,4,5,5- tetramethyl- 1,3,2- dioxaborolan- 2- yl)phenyl] piperidine- 2- carboxylate |

### General Method for the Hydrolysis of Amino Ester Derivatives

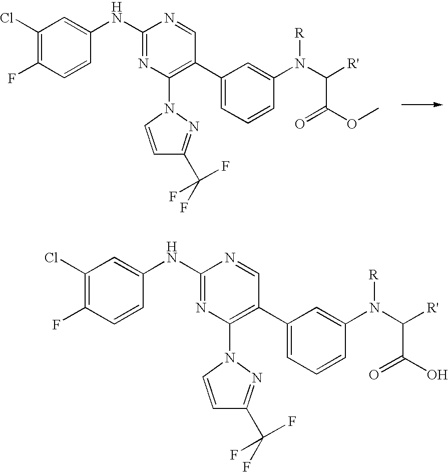

### Method I:

To the amino ester derivative (1 eq) taken in a mixture of tetrahydrofuran and water (3:1), was added Barium hydroxide monohydrate (2 eq) and allowed to stir at room temperature for 12 h. After completion of the reaction, the mixture was then carefully acidified with 1 N HCl and the precipitate formed was filtered, washed with water and dried to yield the product.

### Method II:

To the amino ester derivative (1 eq) taken in a mixture of tetrahydrofuran and water (3:1), was added Sodium hydroxide (2 eq) and allowed to stir at room temperature for 3 h. After completion of the reaction, the mixture was then carefully acidified with 1 N HCl and the precipitate formed was filtered, washed with water and dried to yield the product.

### Method III:

To the amino ester derivative (1 eq) taken in a mixture of acetonitrile and water (3:1), was added 1 N aq. Sodium hydroxide (2.5 eq) and the mixture was heated at 85° C. for 1 h. After completion of the reaction, the mixture was then carefully acidified with 1.5 N HCl and the precipitate formed was filtered, washed with water and dried. It was further dissolved in minimum amount of ethyl acetate, then hexane was added dropwise with constant stirring. The precipitate formed was filtered, washed with water and dried to yield to yield the product.

### Method IV:

To the amino ester derivative (1 eq) taken in a mixture of dioxane and water (3:1), was added Sodium hydroxide (2 eq) and the reaction mixture allowed to stir at room temperature for 3 h. After completion of the reaction, the mixture was then carefully acidified with 1 N HCl, extracted with ethyl acetate. The organic layer was concentrated and the solid obtained was dissolved in minimum amount of CH₂Cl₂. Hexane was added dropwise with constant stirring and the precipitate formed was filtered, washed with water and dried to yield the product.

The compounds in the below table were prepared using this general procedure and the starting material specified.

| | | Mass spectrum and ¹H | |
|---|---|---|---|
| Compound | Structure | NMR | SM |
| Example 598^{c)} | 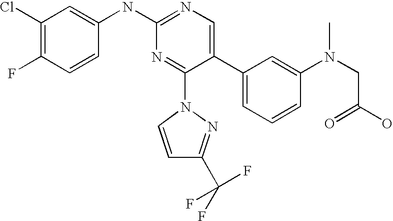 N-(3-{2-[(3-chloro-4- fluorophenyl)amino]-4- [3-(trifluoromethyl)-1H- pyrazol-1-yl]pyrimidin- 5-yl}phenyl)-N- methylglycine | MS(ES): 521 (M + 1) for C₂₃H₁₇ClF₄N₆O₂. 400 MHz, DMSO-d₆: δ 2.88 (s, 3H), 4.03 (s, 2H), 6.42 (m, 2H), 6.61 (d, J = 8.40 Hz, 1H), 6.93 (d, J = 2.80 Hz, 1H), 7.13 (t, J = 7.60 Hz, 1H), 7.40 (t, J = 9.20 Hz, 1H), 7.67-7.70 (m, 1H), 8.05 (s, 1H), 8.17- 8.20 (m, 1H), 8.78 (s, 1H), 10.42 (s, 1H), 12.46 (br s, 1H). | Example 593 methyl N-(3-{2- [(3-chloro-4- fluorophenyl) amino]-4-[3- (trifluoromethyl)- 1H-pyrazol-1- yl]pyrimidin-5- yl}phenyl)-N- methylglycinate |
| Example 599^{d)} | 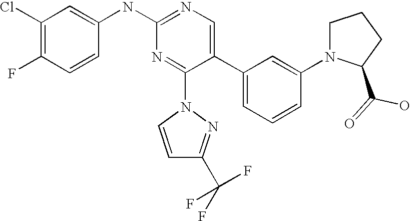 1-(3-{2-[(3-chloro-4- fluorophenyl)amino]-4- [3-(trifluoromethyl)-1H- pyrazol-1-yl]pyrimidin- 5-yl}phenyl)-L-proline | MS(ES): 547 (M + 1) for C₂₅H₁₉ClF₄N₆O_{2.} 400 MHz, DMSO-d₆: δ 1.98-2.06 (m, 3H), 2.19- 2.24 (m, 1H), 3.16-3.22 (m, 1H), 3.33 (m, 1H, merges with water peak), 4.09 (d, J = 9.12 Hz, 1H), 6.31 (s, 1H), 6.35 (d, J = 7.40 Hz, 1H), 6.42 (d, J = 8.24 Hz, 1H), 6.93 (d, J = 2.40 Hz, 1H), 7.12 (t, J = 7.76 Hz, 1H), 7.39 (t, J = 9.04 Hz, 1H), 7.66-7.70 (m, 1H), 8.06 (s, 1H), 8.17 (dd, J = 2.48, 6.72 Hz, 1H), 8.77 (s, 1H), 10.41 (s, 1H), 12.57 (br s, 1H). | Example 594 methyl 1-(3-{2- [(3-chloro-4- fluorophenyl) amino]-4-[3- (trifluoromethyl)- 1H-pyrazol-1- yl]pyrimidin-5- yl}phenyl)-L- prolinate |
| Example 600^{e)} | 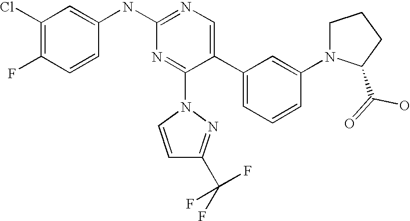 1-(3-{2-[(3-chloro-4- fluorophenyl)amino]-4- [3-(trifluoromethyl)-1H- pyrazol-1-yl]pyrimidin- 5-yl}phenyl)-D-proline | MS(ES): 547 (M + 1) for C₂₅H₁₉ClF₄N₆O_{2.} 400 MHz, DMSO-d₆: δ 1.98-2.06 (m, 3H), 2.21 (m, 1H), 3.20 (m, 1H), 3.34 (m, 1H, merges with water peak), 4.09 (d, J = 8.80 Hz, 1H), 6.31 (s, 1H), 6.36 (d, J = 7.60 Hz, 1H), 6.43 (d, J = 8.00 Hz, 1H), 6.94 (d, J = 2.00 Hz, 1H), 7.12 (t, J = 8.00 Hz, 1H), 7.40 (t, J = 8.80 Hz, 1H), 7.68-7.70 (m, 1H), 8.06 (s, 1H), 8.17- 8.18 (m, 1H), 8.77 (s, 1H), 10.41 (s, 1H), 12.56 (br s, 1H). | Example 595 methyl 1-(3-{2- [(3-chloro-4- fluorophenyl) amino]-4-[3- (trifluoromethyl)- 1H-pyrazol-1- yl]pyrimidin-5- yl}phenyl-D- prolinate |
| Example 601^{f)} | 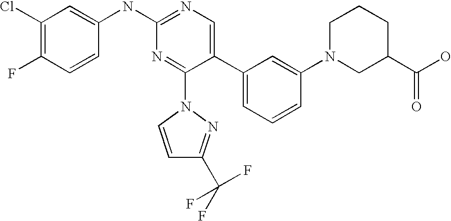 1-(3-{2-[(3-chloro-4- fluorophenyl)amino]-4- [3-(trifluoromethyl)-1H- pyrazol-1-yl]pyrimidin- 5-yl}phenyl)piperidine- 3-carboxylic acid | MS(ES): 561 (M + 1) for C₂₆H₂₁ClF₄N₆O₂. 400 MHz, DMSO-d₆: δ 1.46-1.55 (m, 2H), 1.64- 1.66 (m, 1H), 1.86-1.92 (m, 1H), 2.44-2.50 (m, 1H), 2.67 (m, 1H), 2.85 (dd, J = 9.76, 12.30 Hz, 1H), 3.34 (m, 1H, merges with water peak), 3.50-3.57 (m, 1H), 6.58-6.60 (m, 2H), 6.89-6.93 (m, 2H), 7.19 (t, J = 8.00 Hz, 1H), 7.39 (t, J = 9.12 Hz, 1H), 7.66-7.70 (m, 1H), 8.14- 8.16 (m, 2H), 8.79 (s, 1H), 10.39 (s, 1H), 12.30 (br s, 1H). | Example 596 methyl 1-(3-{2- [(3-chloro-4- fluorophenyl) amino]-4-[3- (trifluoromethyl)- 1H-pyrazol-1- yl]pyrimidin-5- yl}phenyl) piperidine-3- carboxylate |
| Example 602^{f)} | 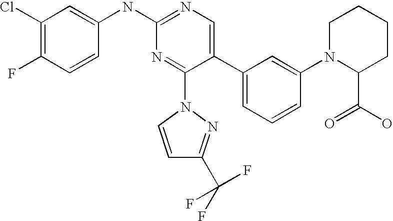 1-(3-{2-[(3-chloro-4- fluorophenyl)amino]-4- [3-(trifluoromethyl)-1H- pyrazol-1-yl]pyrimidin- 5-yl}phenyl)piperidine- 2-carboxylic acid | MS(ES): 561 (M + 1) for C₂₆H₂₁ClF₄N₆O₂. 400 MHz, DMSO-d₆: δ 1.24-1.28 (m, 1H), 1.32- 1.52 (m, 1H), 1.61-1.73 (m, 3H), 2.08 (d, J = 13.16 Hz, 1H), 3.06 (t, J = 2.88 Hz, 1H), 3.38 (m, 1H, merges with water peak), 4.49 (d, J = 2.88 Hz, 1H), 6.50 (d, J = 7.48 Hz, 1H), 6.59 (s, 1H), 6.83 (dd, J = 2.08, 8.46 Hz, 1H), 6.91 (d, J = 2.60 Hz, 1H), 7.14 (t, J = 8.04 Hz, 1H), 7.39 (t, J = 9.08 Hz, 1H), 7.66-7.70 (m, 1H), 8.04 (s, 1H), 8.16 (dd, J = 2.63, 6.67 Hz, 1H), 8.77 (s, 1H), 10.40 (s, 1H), 12.29 (br s, 1H). | Example 597 methyl 1-(3-{2- [(3-chloro-4- fluorophenyl) amino]-4-[3- (trifluoromethyl)- 1H-pyrazol-1- yl]pyrimidin-5- yl}phenyl) piperidine-2- carboxylate |

^{c)}Method I;
^{d)}Method II;
^{e)}Method III;
^{f)}Method IV

### Example 603

### methyl 1-(3-{2-[(3-chloro-4-fluorophenyl)amino]-4-[3-(trifluoromethyl)-1H-pyrazol-1-yl]pyrimidin-5-yl}phenyl)pyrrolidine-3-carboxylate

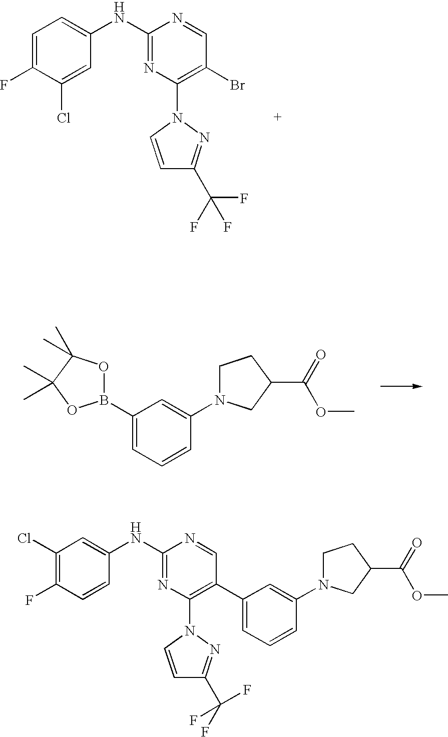

A suspension of 5-bromo-N-(3-chloro-4-fluorophenyl)-4-[3-(trifluoromethyl)-1H-pyrazol-1-yl]pyrimidin-2-amine (Intermediate 115, 0.34 mmol, 0.15 g), the mixture of methyl 1-[3-(4,4,5,5-tetramethyl-1,3,2-dioxaborolan-2-yl)phenyl]pyrrolidine-3-carboxylate and {3-[3-(methoxycarbonyl)pyrrolidin-1-yl]phenyl}boronic acid (Intermediate 208, 0.34 mmol based on the boronic ester, 113 mg), [1,1′-bis(diphenylphosphino)ferrocene]dichloropalladium(II) (0.068 mmol, 50 mg) and sodium carbonate (0.44 mmol, 47 mg) in acetonitrile/water (20 mL:5 mL) was degassed and heated to 90° C. for 15-20 min under inert atmosphere. The solvent was removed under vacuum and the crude mixture was taken in CHCl₃ (50 mL), washed with water, brine, dried over Na₂SO₄, filtered and concentrated. The crude material was purified by silica gel column chromatography using 12% ethyl acetate/hexanes as eluent to yield 65 mg of the title compound.

| | | Mass spectrum and ¹H | |
|---|---|---|---|
| Compound | Structure | NMR | SM |
| Example 603 | 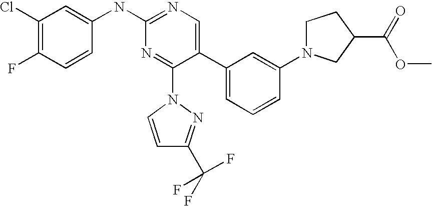 methyl 1-(3-{2-[(3- chloro-4- fluorophenyl)amino]- 4-[3- (trifluoromethyl)- 1H-pyrazol-1- yl]pyrimidin-5- yl}phenyl)pyrrolidine- 3-carboxylate | MS(ES): 561 (M + 1) for C₂₆H₂₁ClF₄N₆O_{2.} 300 MHz, DMSO-d₆: δ 1.13- 1.24 (m, 2H), 2.12-2.19 (m, 2H), 3.17-3.28 (m, 2H), 3.40 (s, 1H), 3.63 (s, 3H), 6.30 (s, 1H), 6.36 (d, J = 7.53 Hz, 1H), 6.51 (d, J = 8.43 Hz, 1H), 6.92 (d, J = 2.52 Hz, 1H), 7.12 (t, J = 15.81 Hz, 1H), 7.38 (t, J = 18.18 Hz, 1H), 7.66-7.71 (m, 1H), 8.07 (s, 1H), 8.16 (dd, J = 2.61, 6.68 Hz, 1H), 8.80 (s, 1H), 10.39 (s, 1H). | Intermediate 208 methyl 1-[3- (4,4,5,5- tetramethyl- 1,3,2- dioxaborolan-2- yl)phenyl] pyrrolidine-3- carboxylate and {3-[3- (methoxy- carbonyl) pyrrolidin-1- yl]phenyl} boronic acid |

### Example 604

### 1-(3-{2-[3-chloro-4-fluorophenyl)amino]-4-[3-(trifluoromethvl)-1H-pyrazol-1-yl]pyrimidin-5-yl}phenyl)pyrrolidine-3-carboxylic acid

To a suspension of Example 603 (0.15 mmol, 85 mg) taken in acetonitrile (6 mL) and water (3 mL), was added NaOH (0.42 mmol, 18 mg) and the mixture heated to 85° C. for 1 h. The reaction mixture was concentrated in vacuo, acidified with 1.5 N HCl and extracted with ethyl acetate (20 mL). The organic layer was washed with brine (10 mL), dried over Na₂SO₄ and concentrated in vacuo. The crude mass was purified by silica gel column chromatography (60-120 mesh) using chloroform and methanol (1%) as eluent to give the title compound (31 mg).

| | | Mass spectrum and ¹H | |
|---|---|---|---|
| Compound | Structure | NMR | SM |
| Example 604 | 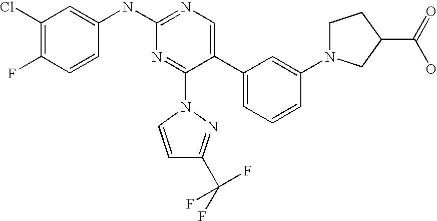 1-(3-{2-[(3-chloro-4- fluorophenyl)amino]- 4-[3-(trifluoromethyl)- 1H-pyrazol-1-yl]pyrimidin-5- yl}phenyl)pyrrolidine- 3-carboxylic acid | MS(ES): 547 (M + 1) for C₂₅H₁₉ClF₄N₆O_{2.} 400 MHz, DMSO-d₆: δ 2.11- 2.18 (m, 2H), 3.12-3.24 (m, 4H), 3.38 (s, 1H, Merges with water peak), 6.32-6.36 (m, 2H), 6.51 (d, J = 8.00 Hz, 1H), 6.92 (d, J = 2.40 Hz, 1H), 7.12 (t, J = 7.80 Hz, 1H), 7.39 (t, J = 9.08 Hz, 1H), 7.66-7.70 (m, 1H), 8.07 (s, 1H), 8.18 (dd, J = 2.56, 6.70 Hz, 1H), 8.81 (s, 1H), 10.40 (s, 1H), 12.40 (br s, 1H). | Example 603 methyl 1-(3-{2- [(3-chloro-4- fluorophenyl) amino]-4-[3- (trifluoromethyl)- 1H-pyrazol-1- yl]pyrimidin-5- yl}phenyl) pyrrolidine-3- carboxylate |

### General procedure for ethyl(2E)-3-(3-{2-[arylamino]-4-[3-(trifluoromethyl)-1H-pyrazol-1-yl]pyrimidin-5-yl}phenyl)prop-2-enoate and ethyl(2E)-3-(3-{2-[arylamino]-4-[5-methyl-3-(trifluoromethyl)-1H-pyrazol-1-yl]pyrimidin-5-yl}phenyl)prop-2-enoate

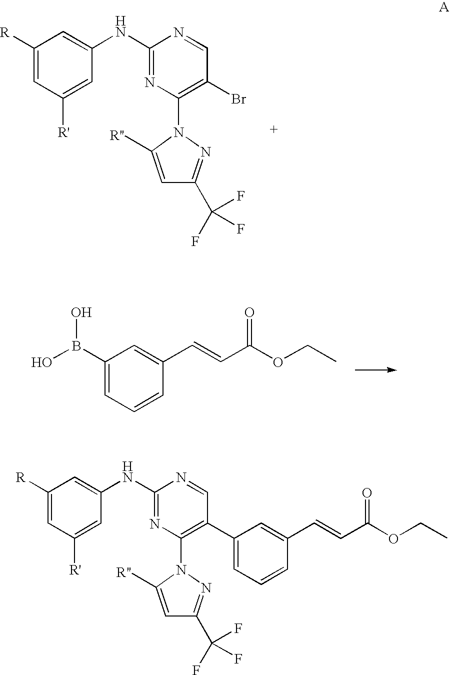

suspension of 5-bromo-N-(aryl)-4-[3-(trifluoromethyl)-1H-pyrazol-1-yl]pyrimidin-2-amine or 5-bromo-N-(aryl)-4-[5-methyl-3-(trifluoromethyl)-1H-pyrazol-1-yl]pyrimidin-2-amine (1 eq), {3-[(1E)-3-ethoxy-3-oxoprop-1-en-1-yl]phenyl}boronic acid (1.1-1.2 eq), [1,1′-bis(diphenylphosphino)ferrocene]dichloropalladium(II) (20 mol %) and sodium carbonate (1 eq) in acetonitrile/water (4:1) was degassed and heated to 90° C. for 15-30 min under inert atmosphere. The solvent was removed in vacuo and the crude mixture taken in CHCl₃ was washed with water, brine, dried over Na₂SO₄, filtered and concentrated. The crude material was purified by silica gel column chromatography using chloroform:methanol (9:1) as eluent to give the product.

| | | Mass spectrum and ¹H | |
|---|---|---|---|
| Compound | Structure | NMR | SM |
| Example 605 | 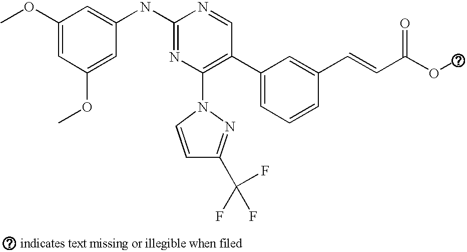 ethyl (2E)-3-(3-{2-[(3,5- dimethoxyphenyl)amino]- 4-[3-(trifluoromethyl)- 1H-pyrazol-1- yl]pyrimidin-5- yl}phenyl)prop-2-enoate | MS(ES): 540 (M + 1) for C₂₇H₂₄F₃N₅O₄. 400 MHz, DMSO-d₆: δ 1.25 (t, J = 7.12 Hz, 3H), 3.73 (s, 6H), 4.18 (q, J = 7.12 Hz, 2H), 6.19-6.20 (m, 1H), 6.59 (d, J = 16.04 Hz, 1H), 6.98 (d, J = 6.04 Hz, 1H), 7.12- 7.15 (m, 3H), 7.37 (t, J = 7.68 Hz, 1H), 7.58 (s, 1H), 7.60 (d, J = 16.08 Hz, 1H), 7.66 (d, J = 7.76 Hz, 1H), 8.31 (br s, 1H), 8.80 (s, 1H), 10.18 (s, 1H). | Intermediate 215 5-bromo-N- (3,5- dimethoxy- phenyl)-4-[3- (trifluoro- methyl)-1H- pyrazol-1- yl]pyrimidin- 2-amine |
| Example 606 | 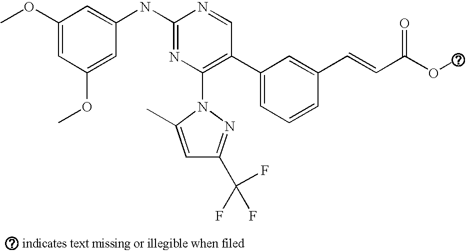 ethyl (2E)-3-(3-{2-[(3,5- dimethoxyphenyl)amino]- 4-[5-methyl-3- (trifluoromethyl)-1H- pyrazol-1-yl]pyrimidin- 5-yl}phenyl)prop-2-enoate | MS(ES): 554 (M + 1) for C₂₈H₂₆F₃N₅O₄. 400 MHz, DMSO-d₆: δ 1.26 (t, J = 7.20 Hz, 3H), 2.17 (s, 3H), 3.72 (s, 6H), 4.19 (q, J = 7.20 Hz, 2H), 6.20-6.21 (m, 1H), 6.55 (d, J = 16.00 Hz, 1H), 6.71 (s, 1H), 7.03-7.07 (m, 3H), 7.36 (t, J = 7.60 Hz, 1H), 7.41 (s, 1H), 7.57 (d, J = 16.00 Hz, 1H), 7.64 (d, J = 8.00 Hz, 1H), 8.96 (s, 1H), 10.23 (s, 1H). | Intermediate 216 5-bromo-N- (3,5- dimethoxy- phenyl)-4-[5- methyl-3- (trifluoro- methyl)-1H- pyrazol-1- yl]pyrimidin- 2-amine |
| Example 607 | 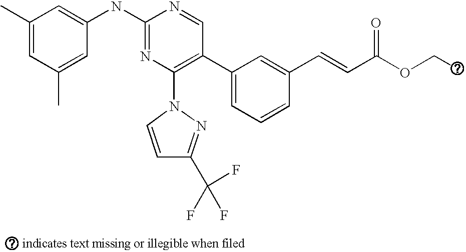 ethyl (2E)-3-(3-{2-[(3,5- dimethylphenyl)amino]- 4-[3-(trifluoromethyl)- 1H-pyrazol-1- yl]pyrimidin-5- yl}phenyl)prop-2-enoate | MS(ES): 508 (M + 1) for C₂₇H₂₄F₃N₅O₂. 300 MHz, DMSO-d₆: δ 1.24 (t, J = 7.08 Hz, 3H), 2.26 (s, 6H), 4.17 (q, J = 7.14 Hz, 2H), 6.59 (d, J = 16.02 Hz, 1H), 6.67 (s, 1H), 6.98 (d, J = 2.46 Hz, 1H), 7.12 (d, J = 7.32 Hz, 1H), 7.36 (t, J = 7.68 Hz, 1H), 7.43 (s, 2H), 7.60 (d, J = 16.65 Hz, 1H), 7.62-7.66 (m, 2H), 8.33 (br s, 1H), 8.77 (s, 1H), 10.08 (s, 1H). | Intermediate 217 5-bromo-N- (3,5- dimethylphenyl)- 4-[3- (trifluoro- methyl)-1H- pyrazol-1- yl]pyrimidin- 2-amine |
| Example 608 | 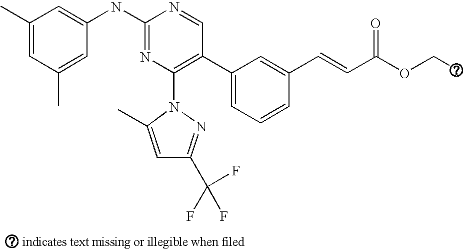 ethyl (2E)-3-(3-{2-[(3,5- dimethylphenyl)amino]- 4-[5-methyl-3- (trifluoromethyl)-1H- pyrazol-1-yl]pyrimidin- 5-yl}phenyl)prop-2- enoate | MS(ES): 522 (M + 1) for C₂₈H₂₆F₃N₅O₂. 300 MHz, DMSO-d₆: δ 1.24 (t, J = 7.11 Hz, 3H), 2.22 (s, 3H), 2.24 (s, 6H), 4.17 (q, J = 7.11 Hz, 2H), 6.54 (d, J = 16.08 Hz, 1H), 6.68-6.70 (m, 2H), 6.99-7.01 (m, 1H), 7.34- 7.39 (m, 4H), 7.55 (d, J = 16.08 Hz, 1H), 7.59-7.63 (m, 1H), 8.92 (s, 1H), 10.11 (br s, 1H). | Intermediate 218 5-bromo-N- (3,5- dimethylphenyl)- 4-[5- methyl-3- (trifluoro- methyl)-1H- pyrazol-1- yl]pyrimidin- 2-amine |
| Example 609 | 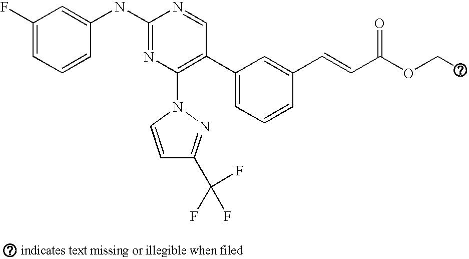 ethyl (2E)-3-(3-{2-[(3- fluorophenyl)amino]-4- [3-(trifluoromethyl)-1H- pyrazol-1-yl]pyrimidin- 5-yl}phenyl)prop-2-enoate | MS(ES): 498 (M + 1) for C₂₅H₁₉F₄N₅O₂. 400 MHz, CDCl₃: δ 1.34 (t, J = 7.12 Hz, 3H), 4.27 (q, J = 7.16 Hz, 2H), 6.41 (d, J = 16.00 Hz, 1H), 6.65 (d, J = 2.64 Hz, 1H), 6.80-6.85 (m, 1H), 7.18 (d, J = 7.80 Hz, 1H), 7.25 (dd, J = 1.12, 8.14 Hz, 1H), 7.33-7.35 (m, 2H), 7.40 (dd, J = 9.28, 16.22 Hz, 1H), 7.45 (br s, 1H), 7.54 (d, J = 7.80 Hz, 1H), 7.67 (d, J = 16.08 Hz, 1H), 7.71 (dt, J = 2.20, 6.74 Hz, 1H), 8.17 (br s, 1H), 8.57 (s, 1H). | Intermediate 219 5-bromo-N- (3- fluorophenyl)- 4-[3- (trifluoro- methyl)-1H- pyrazol-1- yl]pyrimidin- 2-amine |
| Example 610 | 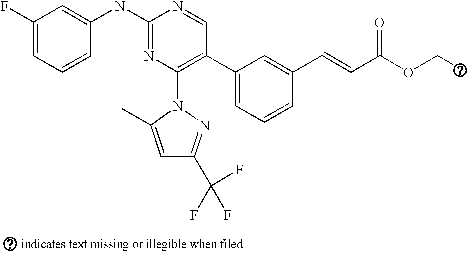 ethyl (2E)-3-(3-{2-[(3- fluorophenyl)amino]-4- [5-methyl-3- (trifluoromethyl)-1H- pyrazol-1-yl]pyrimidin- 5-yl}phenyl)prop-2-enoate | MS(ES): 512 (M + 1) for C₂₆H₂₁F₄N₅O₂. 400 MHz, DMSO-d₆: δ 1.25 (t, J = 7.08 Hz, 3H), 2.20 (s, 3H), 4.18 (q, J = 7.12 Hz, 2H), 6.55 (d, J = 16.04 Hz, 1H), 6.71 (s, 1H), 6.82-6.87 (m, 1H), 7.05 (d, J = 7.92 Hz, 1H), 7.34-7.40 (m, 3H), 7.51 (dd, J = 1.08, 8.20 Hz, 1H), 7.56 (d, J = 16.04 Hz, 1H), 7.64 (d, J = 7.92 Hz, 1H), 7.78 (d, J = 12.08 Hz, 1H), 8.99 (s, 1H), 10.49 (s, 1H). | Intermediate 220 5-bromo-N- (3- fluorophenyl)- 4-[5-methyl- 3- (trifluoro- methyl)-1H- pyrazol-1- yl]pyrimidin- 2-amine |

### Example 611

### ethyl(2E)-3-(3-{2-[(3,5-difluorophenyl)amino]-4-[3-(trifluoromethyl)-1H-pyrazol-1-yl]pyrimidin-5-yl}phenyl)prop-2-enoate

### Example 612

### ethyl(2E)-3-(3-{2-[(3,5-difluorophenyl)amino]-4-[5-methyl-3-(trifluoromethyl)-1H-pyrazol-1-yl]pyrimidin-5-yl}phenyl)prop-2-enoate

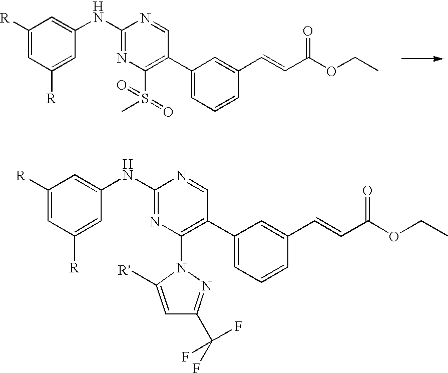

A suspension of either 3-(trifluoromethyl)-1H-pyrazole or 5-methyl-3-(trifluoromethyl)-1H-pyrazole (1.2-1.5 eq), potassium tert-butoxide (1.5 eq) and ethyl(2E)-3-(3-{2-[(3,5-difluorophenyl)amino]-4-(methylsulfonyl)pyrimidin-5-yl}phenyl)prop-2-enoate (Intermediate 223, 1 eq) in DMSO (3 mL) was subjected to microwave irradiation at 130° C. for 1 h. After the reaction was cooled to RT, the mixture was diluted with EtOAc, washed successively with water and brine. The organic layer was dried over sodium sulphate, filtered, concentrated and further purified by silica gel column chromatography using ethyl acetate/hexanes to yield the product. The compounds in the below table were prepared using this procedure and the specified starting material.

| | | Mass spectrum and | |
|---|---|---|---|
| Compound | Structure | ¹H NMR | SM |
| Example 611 | 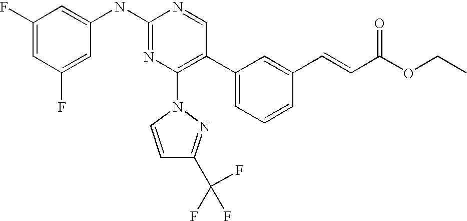 ethyl (2E)-3-(3-{2-[(3,5- difluorophenyl)amino]-4- [3-(trifluoromethyl)-1H- pyrazol-1-yl]pyrimidin-5- yl}phenyl)prop-2-enoate | MS(ES): 516 (M + 1) for C₂₅H₁₈F₅N₅O₂. ¹H NMR (400 MHz, DMSO-d₆) δ 1.25 (t, J = 7.15 Hz, 3H), 4.18 (q, J = 7.03 Hz, 2H), 6.60 (d, J = 16.06 Hz, 1H), 6.85 (tt, J = 2.26, 9.29 Hz, 1H), 7.01 (d, J = 2.51 Hz, 1H), 7.15 (d,J = 7.78 Hz, 1H), 7.38 (t, J = 7.65 Hz, 1H), 7.54-7.64 (m, 4 H), 7.68 (d, J = 7.78 Hz, 1H), 8.36 (d, J = 1.51 Hz, 1H), 8.88 (s, 1H), 10.65 (s, 1H). | Intermediate 223 ethyl (2E)-3-(3- {2-[(3,5- difluorophenyl) amino]-4- (methylsulfonyl) pyrimidin-5- yl}phenyl)prop- 2-enoate |
| Example 612 | 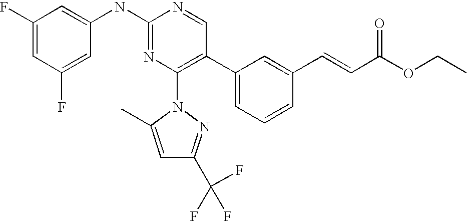 ethyl (2E)-3-(3-{2-[(3,5- difluorophenyl)amino]-4- [5-methyl-3- (trifluoromethyl)-1H- pyrazol-1-yl]pyrimidin-5- yl}phenyl)prop-2-enoate | MS(ES): 530 (M + 1) for C₂₆H₂₀F₅N₅O_{2.} ¹H NMR (400 MHz, DMSO-d₆) δ 1.25 (t, J = 7.15 Hz, 3H), 2.19 (s, 3H), 4.18 (q, J = 7.11 Hz, 2H), 6.55 (d, J = 16.06 Hz, 1H), 6.72 (s, 1H), 6.85 (tt, J = 2.29, 9.25 Hz, 1H), 7.05 (d, J = 8.03 Hz, 1H), 7.37 (t, J = 7.78 Hz, 1H), 7.41 (s, 1H), 7.50-7.60 (m, 3H), 7.65 (d, J = 7.78 Hz, 1H), 9.03 (s, 1H), 10.68 (s, 1H). | Intermediate 223 ethyl (2E)-3-(3- {2-[(3,5- difluorophenyl) amino]-4- (methylsulfonyl) pyrimidin-5- yl}phenyl)prop- 2-enoate |

### Example 613

### ethyl(2E)-3-[3-(2-{[3-(methylsulfonyl)phenyl]amino}-4-[3-(trifluoromethyl)-1H-pyrazol-1-yl]pyrimidin-5-yl)phenyl]prop-2-enoate

### Example 614

### ethyl(2E)-3-[3-(2-{[3-(methylsulfonyl)phenyl]amino}-4-[5-methyl-3-(trifluoromethyl)-1H-pyrazol-1-yl]pyrimidin-5-yl)phenyl]prop-2-enoate

NaH (60% dispersion in mineral oil, 2 eq) was suspended in 1 mL of DMF and stirred for about 5 min at 0° C. Then either 3-(trifluoromethyl)-1H-pyrazole or 5-methyl-3-(trifluoromethyl)-1H-pyrazole (2 eq) in DMF (2 mL) was added dropwise at 0° C. for about 10 min and stirring continued for about 20 min under N₂. Then ethyl(2E)-3-{3-[4-(methylsulfonyl)-2-{[3-(methylsulfonyl)phenyl]amino}pyrimidin-5-yl]phenyl}prop-2-enoate (Intermediate 224, 1 eq) in DMF was added dropwise and the reaction was stirred overnight at room temperature. After completion of the reaction, water was added and the solid obtained was filtered off, dried and purified by silica gel column chromatography using ethyl acetate/hexanes as eluent to yield the product. The compounds in the below table were prepared using this procedure and the specified starting material.

| | | Mass spectrum and | |
|---|---|---|---|
| Compound | Structure | ¹H NMR | SM |
| Example 613 | 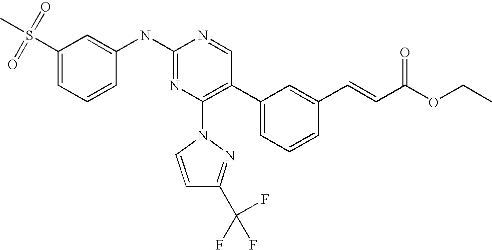 ethyl (2E)-3-[3-(2-{[3- (methylsulfonyl)phenyl] amino}-4-[3- (trifluoromethyl)-1H- pyrazol-1-yl]pyrimidin-5- yl)phenyl]prop-2-enoate | MS(ES): 558 (M + 1) for C₂₆H₂₂F₃N₅O₄S: 400 MHz, DMSO-d₆: δ 1.25 (t, J = 6.96 Hz, 3H), 3.23 (s, 3H), 4.18 (q, J = 7.08 Hz, 2H), 6.61 (d, J = 16.08 Hz, 1H), 7.02 (d, J = 2.52 Hz, 1H), 7.17 (d, J = 7.84 Hz, 1H), 7.38 (t, J = 7.68 Hz, 1H), 7.56- 7.69 (m, 5H), 7.98 (d, J = 8.36 Hz, 1H), 8.49 (br s, 1H), 8.61 (s, 1H), 8.85 (s, 1H), 10.62 (s, 1H). | Intermediate 224 ethyl (2E)-3-{3- [4- (methylsulfonyl)- 2-{[3- (methylsulfonyl) phenyl]amino} pyrimidin-5- yl]phenyl}prop- 2-enoate |
| Example 614 | 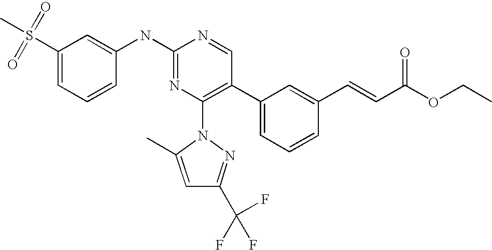 ethyl (2E)-3-[3-(2-{[3- (methylsulfonyl)phenyl] amino}-4-[5-methyl-3- (trifluoromethyl)-1H- pyrazol-1-yl]pyrimidin-5- yl)phenyl]prop-2-enoate | MS(ES): 572 (M + 1) for C₂₇H₂₄F₃N₅O₄S. 400 MHz, DMSO-d₆: δ 1.25 (t, J = 7.04 Hz, 3H), 2.15 (s, 3H), 3.20 (s, 3H), 4.18 (q, J = 7.12 Hz, 2H), 6.57 (d, J = 16.04 Hz,1H), 6.54 (s, 1H), 7.03 (d, J = 3.68 Hz, 1H), 7.36 (t, J = 7.72 Hz, 1H), 7.44 (s, 1H), 7.55-7.66 (m, 4H), 8.01 (d, J = 8.16 Hz, 1H), 8.47 (s, 1H), 9.01 (s, 1H), 10.67 (s, 1H). | Intermediate 224 ethyl (2E)-3-{3- [4- (methylsulfonyl)- 2-{[3- (methylsulfonyl) phenyl]amino} pyrimidin-5- yl]phenyl}prop- 2-enoate |

### Hydrolysis of Carboxylic Esters to Acids

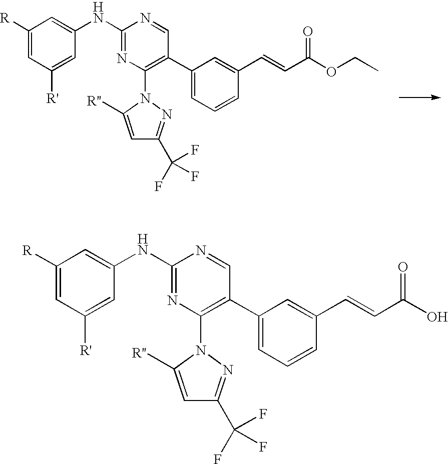

### Example 615

### (2E)-3-(3-{2-[(3,5-dimethoxyphenyl)amino]-4-[3-(trifluoromethyl)-1H-pyrazol-1-yl]pyrimidin-5-yl}phenyl)prop-2-enoic acid

To a suspension of Example 605 (1 eq) taken in dioxane and water (1:1), was added Barium hydroxide monohydrate (2 eq) and the mixture was stirred at RT for 2 days. The reaction mixture was concentrated in vacuo, acidified with 1 N HCl and the precipitate formed was filtered off. The solid was taken in acetonitrile and stirred for 1 h. The solid was then filtered off, washed with dichloromethane and dried to give the title compound.

### Example 616

### (2E)-3-(3-{2-[(3,5-dimethoxyphenyl)amino]-4-[5-methyl-3-(trifluoromethyl)-1H-pyrazol-1-yl]pyrimidin-5-yl}phenyl)prop-2-enoic acid

To a suspension of Example 606 (1 eq) taken in dioxane and water (1:1), was added Barium hydroxide monohydrate (2 eq) and the mixture was warmed to 60° C. for 2 h. The reaction mixture was concentrated in vacuo, acidified with 1 N HCl and the precipitate formed was filtered and dried to give the title compound.

### Example 617

### (2E)-3-(3-{2-[(3,5-dimethylphenyl)amino]-4-[3-(trifluoromethyl)-1H-pyrazol-1-yl]pyrimidin-5-yl}phenyl)prop-2-enoic acid

To a suspension of Example 607 (1 eq) taken in THF and water (2:1), was added Barium hydroxide monohydrate (3 eq) and the mixture was allowed to stir at RT for 2 days and refluxed at 60° C. for 2 days. The reaction mixture was concentrated in vacuo, acidified with 1 N HCl and the precipitate formed was filtered, washed with water and dried to give the title compound.

### Example 618

### (2E)-3-(3-{2-[(3,5-dimethylphenyl)amino]-4-[5-methyl-3-(trifluoromethyl)-1H-pyrazol-1-yl]pyrimidin-5-yl}phenyl)prop-2-enoic acid

To a suspension of Example 608 (1 eq) taken in THF and water (2:1), was added sodium hydroxide (2 eq) and the mixture was heated to 50° C. for 5 days. The reaction mixture was concentrated in vacuo, acidified with 1 N HCl and the precipitate formed was filtered, washed with water and dried to give the title compound.

### Example 619

### (2E)-3-(3-{2-[(3,5-difluorophenyl)amino]-4-[3-(trifluoromethyl)-1H-pyrazol-1-yl]pyrimidin-5-yl}phenyl)prop-2-enoic acid

To a suspension of Example 611 (1 eq) taken in THF and water (2:1), was added Barium hydroxide monohydrate (4 eq) and the mixture was allowed to stir overnight at RT. The reaction mixture was concentrated in vacuo, acidified with 1 N HCl and the precipitate formed was filtered off. The solid was taken in10 mL of dichloromethane and stirred for 1 h. The solid was then filtered off, washed with dichloromethane and dried to give the title compound.

### Example 620

### (2E)-3-(3-{2-[(3,5-difluorophenyl)amino]-4-[5-methyl-3-(trifluoromethyl)-1H-pyrazol-1-yl]pyrimidin-5-yl}phenyl)prop-2-enoic acid

To a suspension of Example 612 (1 eq) taken in dioxane and water (1:1), was added Barium hydroxide monohydrate (4 eq) and the mixture was heated overnight at 70° C. The reaction mixture was concentrated in vacuo, acidified with 1 N HCl and the precipitate formed was filtered off. The solid was taken in dichloromethane and stirred for 1 h. The solid was then filtered off, washed with dichloromethane and dried to give the title compound.

### Example 621

### (2E)-3-(3-{2-[(3-fluorophenyl)amino]-4-[3-(trifluoromethvl)-1H-pyrazol-1-yl]pyrimidin-5-yl}phenyl)prop-2-enoic acid

To a suspension of Example 609 (1 eq) taken in THF and water (1:1), was added lithium hydroxide monohydrate (4 eq) and the mixture was stirred overnight at RT. The reaction mixture was concentrated in vacuo, acidified with 1 N HCl and the precipitate formed was filtered off. The solid was taken in acetonitrile and stirred overnight. The solid was then filtered off, washed with acetonitrile and dried to give the title compound.

### Example 622

### (2E)-3-(3-{2-[(3-fluorophenyl)amino]-4-[5-methyl-3-(trifluoromethyl)-1H-pyrazol-1-yl]pyrimidin-5-yl}phenyl)prop-2-enoic acid

To a suspension of Example 610 (1 eq) taken in THF and water (1:1), was added lithium hydroxide monohydrate (4 eq) and the mixture was stirred at RT for 2 days. The reaction mixture was concentrated in vacuo, acidified with 1 N HCl and the precipitate formed was filtered off. The solid was taken in acetonitrile and stirred for 1 h. The solid was then filtered off, washed with acetonitrile and dried to give the title compound.

### Example 623

### (2E)-3-[3-(2-{[3-(methylsulfonyl)phenyl]amino}-4-[3-(trifluoromethyl)-1H-pyrazol-1-yl]pyrimidin-5-yl)phenyl}prop-2-enoic acid

To a suspension of Example 613 (1 eq) taken in dioxane and water (2:1), was added sodium hydroxide (2 eq) and the mixture was allowed to stir overnight at RT. The reaction mixture was concentrated in vacuo, acidified with 1 N HCl and the precipitate formed was filtered, washed with water and dried to give the title compound.

### Example 624

### (2E)-3-[3-(2-{[3-(methylsulfonyl)phenyl]amino}-4-[5-methyl-3-(trifluoromethyl)-1H-pyrazol-1-yl]pyrimidin-5-yl)phenyl}prop-2-enoic acid

To a suspension of Example 614 (1 eq) taken in dioxane and water (2:1), was added sodium hydroxide (2 eq) and the mixture was allowed to stir overnight at RT. The reaction mixture was concentrated in vacuo, acidified with 1 N HCl and the precipitate formed was filtered, washed with water and dried to give the title compound.

| | | Mass spectrum and ¹H | |
|---|---|---|---|
| Compound | Structure | NMR | SM |
| Example 615 | 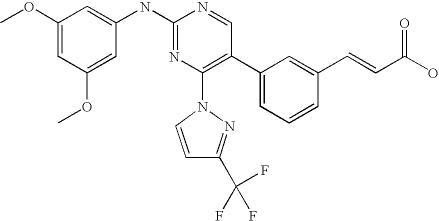 (2E)-3-(3-{2-[(3,5- dimethoxyphenyl)amino]- 4-[3-(trifluoromethyl)- 1H-pyrazol-1- yl]pyrimidin-5- yl}phenyl)prop-2-enoic acid | MS(ES): 512 (M + 1) for C₂₅H₂₀F₃N₅O₄. 400 MHz, DMSO-d₆: δ 3.73 (s, 6H), 6.19 (t, J = 2.13 Hz, 1H), 6.48 (d, J = 16.06 Hz, 1H), 6.98 (d, J = 2.51 Hz, 1H), 7.10-7.17 (m, 3H), 7.37 (t, J = 7.78 Hz, 1H), 7.50-7.57 (m, 2H), 7.63 (d, J = 7.78 Hz, 1H), 8.31 (d, J = 1.76 Hz, 1H), 8.80 (s, 1H), 10.18 (s, 1H), 12.41 (br s, 1H). | Example 605 ethyl (2E)-3- (3-{2-[(3,5- dimethoxy phenyl)amino]- 4-[3- (trifluoro- methyl)-1H- pyrazol-1- yl]pyrimidin- 5- yl}phenyl) prop-2-enoate |
| Example 616 PE-045-02 | 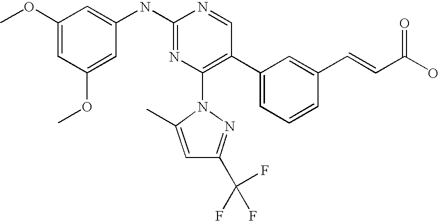 (2E)-3-(3-{2-[(3,5- dimethoxyphenyl)amino]- 4-[5-methyl-3- (trifluoromethyl)-1H- pyrazol-1-yl]pyrimidin- 5-yl}phenyl)prop-2-enoic acid | MS(ES): 526 (M + 1) for C₂₆H₂₂F₃N₅O₄. 400 MHz, DMSO-d₆: δ 2.18 (s, 3H), 3.71 (s, 6 H), 6.20 (s,1H), 6.43 (d, J = 16.06 Hz, 1H), 6.71 (s, 1H), 6.99-7.11 (m, 3H), 7.30-7.39 (m, 2H), 7.49 (d, J = 16.06 Hz, 1H), 7.60 (d, J = 7.78 Hz, 1H), 8.95 (s, 1H), 10.22 (s, 1H), 12.36- 12.48 (m, 1H). | Example 606 ethyl (2E)-3- (3-{2-[(3,5- dimethoxy- phenyl)amino]- 4-[5-methyl- 3- (trifluoro- methyl)-1H- pyrazol-1- yl]pyrimidin- 5- yl}phenyl)- prop-2-enoate |
| Example 617 | 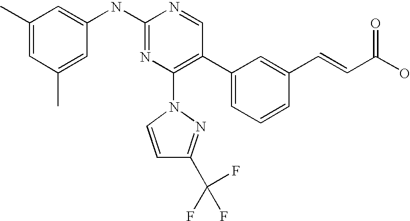 (2E)-3-(3-{2-[(3,5- dimethylphenyl)amino]- 4-[3-(trifluoromethyl)- 1H-pyrazol-1- yl]pyrimidin-5- yl}phenyl)prop-2-enoic acid | MS(ES): 480 (M + 1) for C₂₅H₂₀F₃N₅O₂. 400 MHz, DMSO-d₆: δ 2.27 (s, 6H), 6.47 (d, J = 15.87 Hz, 1H), 6.68 (s, 1H), 6.99 (br s, 1H), 7.12 (d, J = 7.32 Hz, 1H), 7.36 (t, J = 7.63 Hz, 1H), 7.41- 7.55 (m, 4H), 7.61 (d, J = 7.93 Hz, 1H), 8.33 (br s, 1H), 8.78 (s, 1H), 10.09 (s, 1H), 12.5 (br s, 1H). | Example 607 ethyl (2E)-3- (3-{2-[(3,5- dimethyl- phenyl)amino]-4- [3- (trifluoro- methyl)-1H- pyrazol-1- yl]pyrimidin- 5- yl}phenyl) prop-2-enoate |
| Example 618 | 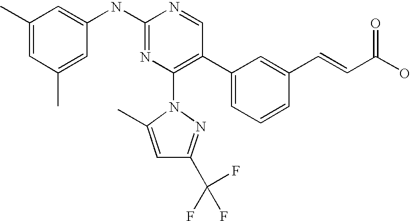 (2E)-3-(3-{2-[(3,5- dimethylphenyl)amino]- 4-[5-methyl-3- (trifluoromethyl)-1H- pyrazol-1-yl]pyrimidin- 5-yl}phenyl)prop-2-enoic acid | MS(ES): 494 (M + 1) for C₂₆H₂₂F₃N₅O₂. 400 MHz, DMSO-d₆: δ 2.24 (s, 3H), 2.25 (s, 6H), 6.45 (d, J = 15.96 Hz, 1H), 6.68 (s, 1H), 6.71 (s, 1H), 7.01 (d, J = 7.80 Hz, 1H), 7.32-7.35 (m, 2H), 7.39 (s, 2H), 7.42 (d, J = 16.00 Hz, 1H), 7.57 (d, J = 7.84 Hz, 1H), 8.92 (s, 1H), 10.11 (s, 1H). | Example 608 ethyl (2E)-3- (3-{2-[(3,5- dimethyl- phenyl)amino]-4- [5-methyl-3- (trifluoro- methyl)-1H- pyrazol-1- yl]pyrimidin- 5- yl}phenyl) prop-2-enoate |
| Example 619 | 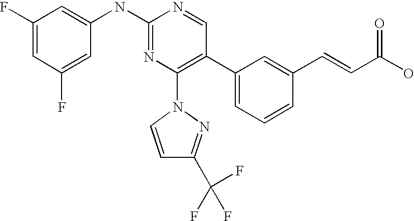 (2E)-3-(3-{2-[(3,5- difluorophenyl)amino]-4- [3-(trifluoromethyl)-1H- pyrazol-1-yl]pyrimidin- 5-yl}phenyl)prop-2-enoic acid | MS(ES): 488 (M + 1) for C₂₃H₁₄F₅N₅O_{2.} 400 MHz, DMSO-d₆: δ 6.48 (d, J = 16.00 Hz, 1H), 6.85 (t, J = 9.20 Hz, 1H), 7.01 (d, J = 2.32 Hz, 1H), 7.15 (d, J = 7.76 Hz, 1H), 7.32 (t, J = 7.68 Hz, 1H), 7.52-7.66 (m, 5H), 8.35 (br s, 1H), 8.88 (s, 1H), 10.64 (s, 1H), 12.50 (br s, 1H). | Example 611 ethyl (2E)-3- (3-{2-[(3,5- difluoro- phenyl)amino]-4- [3- (trifluoro- methyl)-1H- pyrazol-1- yl]pyrimidin- 5- yl}phenyl) prop-2-enoate |
| Example 620 | 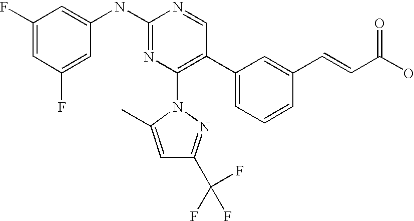 (2E)-3-(3-{2-[(3,5- difluorophenyl)amino]-4- [5-methyl-3- (trifluoromethyl)-1H- pyrazol-1-yl]pyrimidin- 5-yl}phenyl)prop-2-enoic acid | MS(ES): 502 (M + 1) for C₂₄H₁₆F₅N₅O₂. 400 MHz, DMSO-d₆: δ 2.21 (s, 3H), 6.45 (d, J = 16.04 Hz, 1H), 6.73 (s, 1H), 6.83-6.88 (m, 1H), 7.06 (d, J = 7.80 Hz, 1H), 7.35-7.39 (m, 2H), 7.48- 7.56 (m, 3H), 7.62 (d, J = 7.84 Hz, 1H), 9.03 (s, 1H), 10.69 (s, 1H), 12.53 (br s, 1H). | Example 612 ethyl (2E)-3- (3-{2-[(3,5- difluoro- phenyl)amino]-4- [5-methyl-3- (trifluoro- methyl)-1H- pyrazol-1- yl]pyrimidin- 5- yl}phenyl) prop-2-enoate |
| Example 621 | 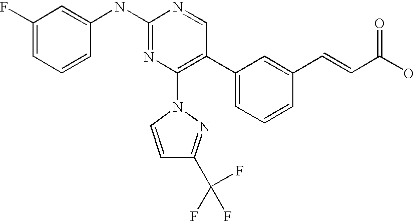 (2E)-3-(3-{2-[(3- fluorophenyl)amino]-4- [3-(trifluoromethyl)-1H- pyrazol-1-yl]pyrimidin- 5-yl}phenyl)prop-2-enoic acid | MS(ES): 470 (M + 1) for C₂₃H₁₅F₄N₅O₂. 400 MHz, DMSO-d₆: δ 6.47 (d, J = 16.04 Hz, 1H), 6.84 (td, J = 2.16, 11.84 Hz, 1H), 7.00 (d, J = 2.48 Hz, 1H), 7.14 (d, J = 7.68 Hz, 1H), 7.34-7.39 (m, 2H), 7.52-7.56 (m, 3H), 7.63 (d, J = 7.84 Hz, 1H), 7.81 (d, J = 12.16 Hz, 1H), 8.38 (br s, 1H), 8.83 (s, 1H), 10.45 (s, 1H), 12.41 (br s, 1H). | Example 609 ethyl 92E)-3- (3-{2-[(3- fluorophenyl) amino]-4-[3- (trifluoro- methyl)-1H- pyrazol-1- yl]pyrimidin- 5- yl}phenyl) prop-2-enoate |
| Example 622 | 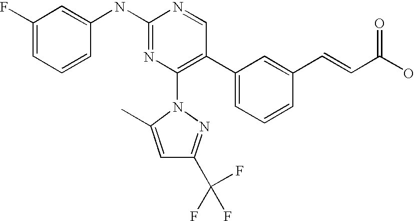 (2E)-3-(3-{2-[(3- fluorophenyl)amino]-4- [5-methyl-3- (trifluoromethyl)-1H- pyrazol-1-yl]pyrimidin- 5-yl}phenyl)prop-2-enoic acid | MS(ES): 484 (M + 1) for C₂₄H₁₇F₄N₅O₂. 400 MHz, DMSO-d₆: δ 2.22 (s, 3H), 6.44 (d, J = 15.60 Hz, 1H), 6.73 (s, 1H), 6.85 (t, J = 8.00 Hz, 1H), 7.05 (d, J = 7.60 Hz, 1H), 7.34-7.40 (m, 3H), 7.48-7.52 (m, 2H), 7.61 (d, J = 8.00 Hz, 1H), 7.78 (d, J = 12.00 Hz, 1H), 8.99 (s, 1H), 10.50 (s, 1H), 12.41 (br s, 1H). | Example 610 ethyl (2E)-3- (3-{2-[(3- fluorophenyl) amino]-4-[5- methyl-3- (trifluoro- methyl)-1H- pyrazol-1- yl]pyrimidin- 5- yl}phenyl) prop-2-enoate |
| Example 623 | 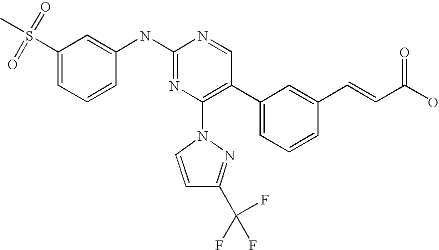 (2E)-3-[3-(2-{[3- (methylsulfonyl)phenyl]- amino}-4-[3- (trifluoromethyl)-1H- pyrazol-1-yl]pyrimidin- 5-yl)phenyl]prop-2-enoic acid | MS(ES): 530 (M + 1) for C₂₄H₁₈F₃N₅O₄S. 400 MHz, DMSO-d₆: δ 3.23 (s, 3H), 6.49 (d, J = 16.00 Hz, 1H), 7.02 (d, J = 2.52 Hz, 1H), 7.17 (d, J = 7.72 Hz, 1H), 7.38 (t, J = 7.72 Hz, 1H), 7.53-7.65 (m, 5H), 7.98 (d, J = 8.00 Hz, 1H), 8.49 (br s, 1H), 8.61 (br s, 1H), 8.85 (s, 1H), 10.64 (s, 1H), 12.41 (br s, 1H). | Example 613 ethyl (2E)-3- [3-(2-{[3- (methyl- sulfonyl) phenyl] amino}-4-[3- (trifluoro- methyl)-1H- pyrazol-1- yl]pyrimidin- 5- yl)phenyl] prop-2-enoate |
| Example 624 | 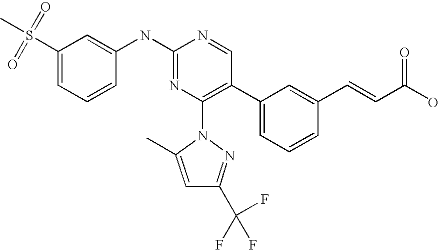 (2E)-3-[3-(2-{[3- (methylsulfonyl)phenyl] amino}-4-[5-methyl-3- (trifluoromethyl)-1H- pyrazol-1-yl]pyrimidin- 5-yl)phenyl]prop-2-enoic acid | MS(ES): 544 (M + 1) for C₂₅H₂₀F₃N₅O₄S. 400 MHz, DMSO-d₆: δ 2.29 (s, 3H), 3.20 (s, 3H), 6.45 (d, J = 16.00 Hz, 1H), 6.74 (s, 1H), 7.04 (d, J = 7.60 Hz, 1H), 7.36 (t, J = 7.72 Hz, 1H), 7.38 (br s, 1H), 7.50 (d, J = 15.96 Hz, 1H), 7.56-7.65 (m, 3H), 8.01 (d, J = 7.96 Hz, 1H), 8.47 (br s, 1H), 9.01 (s, 1H), 10.66 (s, 1H), 12.44 (br s, 1H). | Example 613 ethyl (2E)-3- [3-(2-{[3- (methyl- sulfonyl) phenyl]- amino}-4-[5- methyl-3- (trifluoro- methyl)-1H- pyrazol-1- yl]pyrimidin- 5- yl)phenyl] prop-2-enoate |

### General procedure for the synthesis of ethyl 5-{2-[arylamino]-4-[1H-azol-1-yl]pyrimidin-5-yl}pyridine-3-carboxylate

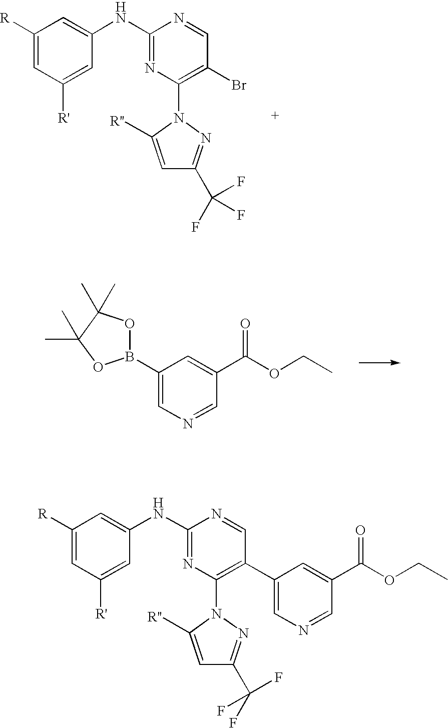

A suspension of either 5-bromo-N-(aryl)-4-[3-(trifluoromethyl)-1H-pyrazol-1-yl]pyrimidin-2-amine or 5-bromo-N-(aryl)-4-[5-methyl-3-(trifluoromethyl)-1H-pyrazol-1-yl]pyrimidin-2-amine (1 eq), ethyl 5-(4,4,5,5-tetramethyl-1,3,2-dioxaborolan-2-yl)pyridine-3-carboxylate (1.1-1.2 eq), [1,1′-bis(diphenylphosphino)ferrocene]-dichloropalladium(II) (10-20 mol %) and sodium carbonate (1-2 eq) in acetonitrile/water (4:1) was degassed and heated to 90° C. for 15-30 min under inert atmosphere. The solvent was removed in vacuo and the crude mixture taken in CHCl₃ was washed with water, brine, dried over Na₂SO₄, filtered and concentrated. The crude material was purified by silica gel column chromatography using 30% ethyl acetate/hexanes as eluent to give the product. The compounds in the below table were prepared using this method and the specified starting material.

| | | Mass spectrum and ¹H | |
|---|---|---|---|
| Compound | Structure | NMR | SM |
| Example 625 | 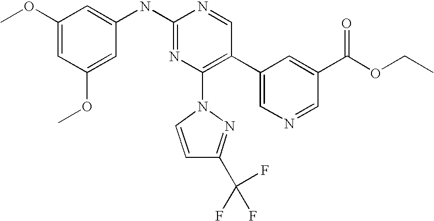 Ethyl 5-{2-[(3,5- dimethoxyphenyl)amino]- 4-[3-(trifluoromethyl)- 1H-pyrazol-1- yl]pyrimidin-5- yl}pyridine-3- carboxylate | MS(ES): 515 (M + 1) for C₂₄H₂₁F₃N₆O₄. 400 MHz, DMSO-d₆: δ 1.30 (t, J = 7.08 Hz, 3H), 3.75 (s, 6H), 4.33 (q, J = 7.12 Hz, 2H), 6.22 (t, J = 2.20 Hz, 1H), 7.05 (d, J = 2.64 Hz, 1H), 7.09 (s, 1H), 7.10 (s, 1H), 8.03 (t, J = 2.12 Hz, 1H), 8.53 (d, J = 1.72 Hz, 1H), 8.69 (d, J = 2.24 Hz, 1H), 8.82 (s, 1H), 9.02 (s, 1H), 10.24 (s, 1H). | Intermediate 215 5-bromo-N-(3,5- dimethoxyphenyl)- 4-[3- (trifluoromethyl)- 1H-pyrazol-1- yl]pyrimidin-2- amine |
| Example 626 | 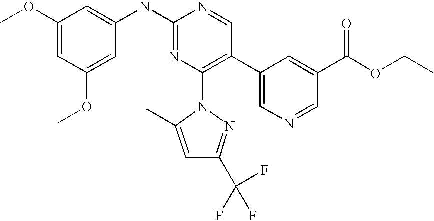 Ethyl 5-{2-[(3,5- dimethoxyphenyl)amino]- 4-[5-methyl-3- (trifluoromethyl)-1H- pyrazol-1-yl]pyrimidin- 5-yl}pyridine-3- carboxylate | MS(ES): 529 (M + 1) for C₂₅H₂₃F₃N₆O₄. 400 MHz, DMSO-d₆: δ 1.30 (t, J = 7.20 Hz, 3H), 2.40 (s, 3H), 3.73 (s, 6H), 4.31 (q, J = 7.20 Hz, 2H), 6.23 (t, J = 2.00 Hz, 1H), 6.78 (s, 1H), 7.05 (m, 2H), 7.80 (t, J = 2.00 Hz, 1H), 8.61 (d, J = 2.40 Hz, 1H), 8.98 (d, J = 1.60 Hz, 1H), 8.99 (s, 1H), 10.27 (s, 1H). | Intermediate 216 5-bromo-N-(3,5- dimethoxyphenyl)- 4-[5-methyl-3- (trifluoromethyl)- 1H-pyrazol-1- yl]pyrimidin-2- amine |
| Example 627 | 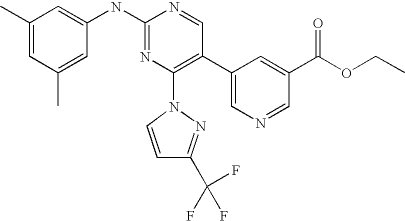 Ethyl 5-{2-[(3,5- dimethoxyphenyl)amino]- 4-[3-(trifluoromethyl)- 1H-pyrazol-1- yl]pyrimidin-5- yl}pyridine-3- carboxylate | MS(ES): 483 (M + 1) for C₂₄H₂₁F₃N₆O₂. 400 MHz, DMSO-d₆: δ 1.31 (t, J = 7.20 Hz, 3H), 2.28 (s, 6H), 4.33 (q, J = 6.80 Hz, 2H), 6.71 (s, 1H), 7.05 (d, J = 2.80 Hz, 1H), 7.42 (s, 2H), 8.03 (t, J = 2.00 Hz,1H), 8.54 (s, 1H), 8.69 (d, J = 2.40 Hz, 1H), 8.80 (s, 1H), 9.02 (d, J = 2.00 Hz, 1H), 10.15 (br s, 1H). | Intermediate 217 5-bromo-N-(3,5- dimethylphenyl)- 4-[3- (trifluoromethyl)- 1H-pyrazol-1- yl]pyrimidin-2- amine |
| Example 628 | 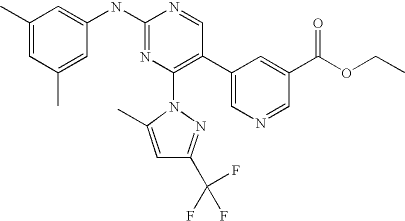 ethyl 5-{2-[(3,5- dimethylphenyl)amino]- 4-[5-methyl-3- (trifluoromethyl)-1H- pyrazol-1-yl]pyrimidin- 5-yl}pyridine-3- carboxylate | MS(ES): 497 (M + 1) for C₂₅H₂₃F₃N₆O₂. 400 MHz, DMSO-d₆: δ 1.30 (t, J = 7.20 Hz, 3H), 2.28 (s, 6H), 2.44 (s, 3H), 4.31 (q, J = 6.80 Hz, 2H), 6.71 (s, 1H), 6.77 (s, 1H), 7.37 (s, 2H), 7.79 (t, J = 2.00 Hz, 1H), 8.60 (d, J = 2.00 Hz, 1H), 8.96 (s, 1H), 8.97 (d, J = 2.00 Hz, 1H), 10.17 (br s, 1H). | Intermediate 218 5-bromo-N-(3,5- dimethylphenyl)- 4-[5-methyl-3- (trifluoromethyl)- 1H-pyrazol-1- yl]pyrimidin-2- amine |
| Example 629 | 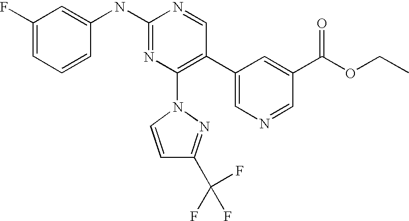 ethyl 5-{2-[(3- fluorophenyl)amino]-4- [3-(trifluoromethyl)-1H- pyrazol-1-yl]pyrimidin- 5-yl}pyridine-3- carboxylate | MS(ES): 473 (M + 1) for C₂₂H₁₆F₄N₆O₂. 400 MHz, DMSO-d₆: δ 1.30 (t, J = 7.08 Hz, 3H), 4.33 (q, J = 7.08 Hz, 2H), 6.86 (td, J = 2.36, 8.38 Hz, 1H), 7.05 (d, J = 2.60 Hz, 1H), 7.38 (dd, J = 8.08, 15.24 Hz, 1H), 7.56 (d, J = 8.24 Hz, 1H), 7.77 (d, J = 12.04 Hz, 1H), 8.03 (t, J = 2.08 Hz, 1H), 8.57 (br s, 1H), 8.69 (t, J = 2.12 Hz, 1H), 8.84 (s, 1H), 9.02 (d, J = 1.92 Hz, 1H), 10.49 (s, 1H). | Intermediate 219 5-bromo-N-(3- fluorophenyl)-4- [3- (trifluoromethyl)- 1H-pyrazol-1- yl]pyrimidin-2- amine |
| Example 630 | 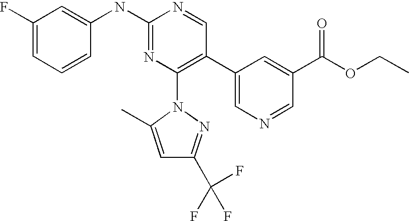 ethyl 5-{2-[(3- fluorophenyl)amino]-4- [5-methyl-3- (trifluoromethyl)-1H- pyrazol-1-yl]pyrimidin- 5-yl}pyridine-3- carboxylate | MS(ES): 487 (M + 1) for C₂₃H₁₈F₄N₆O₂. 400 MHz, DMSO-d₆: δ 1.30 (t, J = 7.20 Hz, 3H), 2.42 (s, 3H), 4.32 (q, J = 7.20 Hz, 2H), 6.79 (s, 1H), 6.87 (td, J = 2.00, 8.20 Hz, 1H), 7.39 (dd, J = 8.40, 15.20 Hz, 1H), 7.52 (d, J = 8.40 Hz, 1H), 7.76-7.79 (m, 1H), 7.81 (t, J = 2.00 Hz, 1H), 8.62 (d,J = 2.00 Hz, 1H), 8.99 (d, J = 2.00 Hz, 1H), 9.03 (s, 1H), 10.54 (s, 1H). | Intermediate 220 5-bromo-N-(3- fluorophenyl)-4- [5-methyl-3- (trifluoromethyl)- 1H-pyrazol-1- yl]pyrimidin-2- amine |

### Example 631

### ethyl 5-{2-[(3,5-difluorophenyl)amino]-4-[3-(trifluoromethyl)-1H-pyrazol-1-yl]pyrimidin-5-yl}pyridine-3-carboxylate

### Example 632

### ethyl 5-{2-[(3,5-difluorophenyl)amino]-4-[5-methyl-3-(trifluoromethyl)-1H-pyrazol-1-yl]pyrimidin-5-yl}pyridine-3-carboxylate

A suspension of either 3-(trifluoromethyl)-1H-pyrazole or 5-methyl-3-(trifluoromethyl)-1H-pyrazole (1.2-1.5 eq), potassium tert-butoxide (1 eq) and ethyl 5-{2-[(3,5-difluorophenyl)amino]-4-(methylsulfonyl)pyrimidin-5-yl}pyridine-3-carboxylate Intermediate 225 (1 eq) in DMSO was subjected to microwave irradiation at 130° C. for 1 h. After the reaction was cooled to RT, the mixture was diluted with EtOAc, washed successively with water and brine. The organic layer was dried over sodium sulphate, filtered, concentrated and further purified by silica gel column chromatography using ethyl acetate/hexanes to yield the product. The compounds in the below table were prepared using this method and the specified starting material.

| | | Mass spectrum and ¹H | |
|---|---|---|---|
| Compound | Structure | NMR | SM |
| Example 631 | 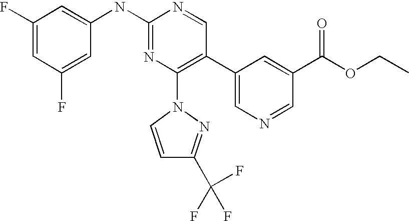 ethyl 5-{2-[(3,5- difluorophenyl)amino]- 4-[3-(trifluoromethyl)- 1H-pyrazol-1- yl]pyrimidin-5- yl}pyridine-3- carboxylate | MS(ES): 491 (M + 1) for C₂₂H₁₅F₅N₆O₂. 400 MHz, DMSO-d₆: δ 1.31 (t, J = 7.20 Hz, 3H), 4.34 (q, J = 7.20 Hz, 2H), 6.88 (m, 1H), 7.07 (d, J = 2.80 Hz, 1H), 7.58 (dd, J = 2.00, 10.00 Hz, 2H), 8.05 (t, J = 2.00 Hz, 1H), 8.57 (d, J = 1.20 Hz, 1H), 8.70 (d, J = 2.40 Hz, 1H), 8.90 (s, 1H), 9.04 (d, J = 2.00 Hz, 1H), 10.70 (s, 1H). | Intermediate 225 ethyl 5-{2-[(3,5- difluorophenyl) amino]-4- (methylsulfonyl) pyrimidin-5- yl}pyridine-3- carboxylate |
| Example 632 | 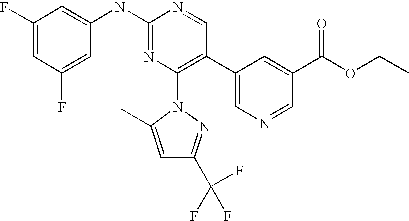 ethyl 5-{2-[(3,5- difluorophenyl)amino]- 4-[5-methyl-3- (trifluoromethyl)-1H- pyrazol-1-yl]pyrimidin- 5-yl}pyridine-3- carboxylate | MS(ES): 505 (M + 1) for C₂₃H₁₇F₅N₆O₂. 400 MHz, DMSO-d₆: δ 1.31 (t, J = 7.08 Hz, 3H), 2.41 (s, 3H), 4.31 (q, J = 7.08 Hz, 2H), 6.79 (s, 1H), 6.87 (t, J = 9.28 Hz, 1H), 7.54 (dd, J = 2.00, 9.96 Hz, 2H), 7.82 (t, J = 2.16 Hz, 1H), 8.62 (d, J = 2.28 Hz, 1H), 8.99 (d, J = 2.00 Hz, 1H), 9.06 (s, 1H), 10.70 (s, 1H). | Intermediate 225 ethyl 5-{2-[(3,5- difluorophenyl) amino]-4- (methylsulfonyl) pyrimidin-5- yl}pyridine-3- carboxylate |

### Example 633

### ethyl 5-(2-{[3-(methylsulfonyl)phenyl]amino}-4-[3-(trifluoromethyl)-1H-pyrazol-1-yl]pyrimidin-5-yl)pyridine-3-carboxylate

### Example 634

### ethyl 5-(2-{[3-(methylsulfonyl)phenyl]amino}-4-[5-methyl-3-(trifluoromethyl)-1H-pyrazol-1-yl]pyrimidin-5-yl)pyridine-3-carboxylate

NaH (60% dispersion in mineral oil, 2 eq) was dissolved in 1 mL of DMF and stirred for about 5 min at 0° C. Then either 3-(trifluoromethyl)-1H-pyrazole or 5-methyl-3-(trifluoromethyl)-1H-pyrazole (2 eq) in DMF (2 mL) was added dropwise at 0° C. for about 10 min and stirring continued for about 20 min under N₂. Then ethyl 5-[4-(methylsulfonyl)-2-{[3-(methylsulfonyl)phenyl]amino}pyrimidin-5-yl]pyridine-3-carboxylate (Intermediate 226, 1 eq) in DMF was added dropwise and the reaction was stirred overnight at room temperature. Water was added and the solid obtained was filtered off, dried and purified by silica gel column chromatography using ethyl acetate/hexanes as eluent to yield the product. The compounds in the below table were prepared using this method and the specified starting material.

| | | Mass spectrum and ¹H | |
|---|---|---|---|
| Compound | Structure | NMR | SM |
| Example 633 | 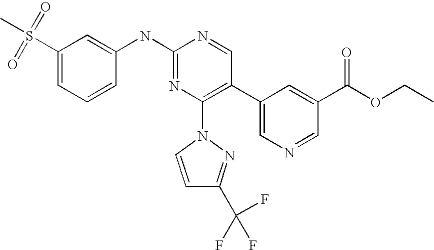 ethyl 5-(2-{[3- (methylsulfonyl)phenyl] amino}-4-[3- (trifluoromethyl)-1H- pyrazol-1-yl]pyrimidin- 5-yl)pyridine-3- carboxylate | MS(ES): 533 (M + 1) for C₂₃H₁₉F₃N₆O₄S. 400 MHz, DMSO-d₆: δ 1.30 (t, J = 7.08 Hz, 3H), 3.24 (s, 3H), 4.34 (q, J = 7.08 Hz, 2H), 7.07 (d, J = 2.64 Hz, 1H), 7.58-7.61 (m, 1H), 7.65 (t, J = 7.88 Hz, 1H), 7.97 (d, J = 7.92 Hz, 1H), 8.10 (t, J = 2.08 Hz, 1H), 8.63 (br s, 1H), 8.67 (br s, 1H), 8.72 (d, J = 2.16 Hz, 1H), 8.86 (s, 1H), 9.04 (d, J = 1.92 Hz, 1H), 10.70 (br s, 1H). | Intermediate 226 ethyl 5-[4- (methylsulfonyl)- 2-{[3- (methylsulfonyl) phenyl]amino} pyrimidin-5- yl]pyridine-3- carboxylate |
| Example 634 | 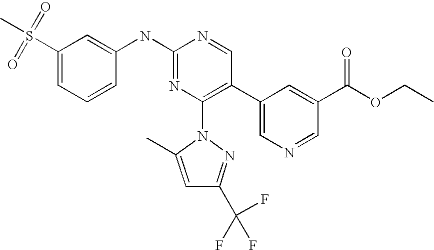 ethyl 5-(2-{[3- (methylsulfonyl)phenyl] amino}-4-[5-methyl-3- (trifluoromethyl)-1H- pyrazol-1-yl]pyrimdin- 5-yl)pyridine-3- carboxylate | MS(ES): 547 (M + 1) for C₂₄H₂₁F₃N₆O₄S. 400 MHz, DMSO-d₆: δ 1.32 (t, J = 7.08 Hz, 3H), 2.48 (s, 3H), 3.21 (s, 3H), 4.31 (q, J = 7.12 Hz, 2H), 6.80 (s, 1H), 7.58-7.60 (m, 1H), 7.64 (t, J = 7.88 Hz, 1H), 7.82 (t, J = 2.12 Hz, 1H), 8.02 (d, J = 7.96 Hz, 1H), 8.47 (t, J = 1.76 Hz, 1H), 8.62 (d, J = 2.24 Hz, 1H), 8.99 (d, J = 1.96 Hz, 1H), 9.04 (s, 1H), 10.70 (br s, 1H). | Intermediate 226 ethyl 5-[4- (methylsulfonyl)- 2-{[3- (methylsulfonyl) phenyl]amino}- pyrimidin-5- yl]pyridine-3- carboxylate |

### Example 635

### 5-{2-[(3,5-Dimethoxyphenyl)amino]-4-[3-(trifluoromethyl)-1H-pyrazol-1-yl]pyrimidin-5-yl}pyridine-3-carboxylic acid

To a suspension of Example 625 (1 eq) taken in dioxane and water (1:1) was added barium hydroxide monohydrate (2 eq) and the mixture was stirred at RT for 2 days. The reaction mixture was concentrated in vacuo, acidified with 1 N HCl and the precipitate formed was filtered, washed with water and dried to give the title compound.

### Example 636

### 5-{2-[(3,5-Dimethoxyphenyl)amino]-4-[5-methyl-3-(trifluoromethyl)-1H-pyrazol-1-yl]pyrimidin-5-yl}hyridine-3-carboxylic acid

To a suspension of Example 626 (1 eq) taken in dioxane and water (1:1), was added Barium hydroxide monohydrate (2 eq) and the mixture was stirred at RT for 24 h. The reaction mixture was concentrated in vacuo, acidified with 1 N HCl and the precipitate formed was filtered and dried to give the title compound.

### Example 637

### 5-{2-[(3,5-dimethylphenyl)amino]-4-[3-(trifluoromethyl)-1H-pyrazol-1-yl]pyrimidin-5-yl}pyridine-3-carboxylic acid

To a suspension of Example 627 (1 eq) taken in THF and water (1:1), was added sodium hydroxide (2 eq) and the mixture was stirred at RT for 2 days. The reaction mixture was concentrated in vacuo, acidified with 1 N HCl and the precipitate formed was filtered, washed with water and dried to give the title compound.

### Example 638

### 5-{2-[(3,5-dimethylphenyl)amino]-4-[5-methyl-3-(trifluoromethyl)-1H-pyrazol-1-yl]pyrimidin-5-yl}pyridine-3-carboxylic acid

To a suspension of Example 628 (1 eq) taken in THF and water (1:1), was added sodium hydroxide (2 eq) and the mixture was heated to 50° C. for 5 days. The reaction mixture was concentrated in vacuo, acidified with 1 N HCl and the precipitate formed was filtered, washed with water and dried to give the title compound.

### Example 639

### 5-{2-[(3,5-difluorophenyl)amino]-4-[3-(trifluoromethyl)-1H-pyrazol-1-yl]pyrimidin-5-yl}pyridine-3-carboxylic acid

To a suspension of Example 631 (1 eq) taken in THF and water (3:1), was added Barium hydroxide monohydrate (2 eq) and the mixture was stirred at RT for 1 h. The reaction mixture was concentrated in vacuo, acidified with 1 N HCl and the precipitate formed was filtered, washed with water and dried to give the title compound.

### Example 640

### 5-{2-[(3,5-difluorophenyl)amino]-4-[5-methyl-3-(trifluoromethyl)-1H-pyrazol-1-yl]pyrimidin-5-yl}pyridine-3-carboxylic acid

To a suspension of Example 632 (1 eq) taken in THF and water (2:1), was added sodium hydroxide (2 eq) and the mixture was allowed to stir at RT for 2 h. The reaction mixture was concentrated in vacuo, acidified with 1 N HCl and the precipitate formed was filtered, washed with water and dried to give the title compound.

### Example 641

### 5-{2-[(3-fluorophenyl)amino]-4-[3-(trifluoromethyl)-1H-pyrazol-1-yl]pyrimidin-5-yl}pyridine-3-carboxylic acid

To a suspension of Example 629 (1 eq) taken in THF and water (1:1), was added lithium hydroxide monohydrate (4 eq) and the mixture was stirred for 30′ at RT. The reaction mixture was concentrated in vacuo, acidified with 1 N HCl and the precipitate formed was stirred with acetonitrile. It was then filtered, washed with acetonitrile and dried to give the title compound.

### Example 642

### 5-{2-[(3-fluorophenyl)amino]-4-[5-methyl-3-(trifluoromethyl)-1H-pyrazol-1-yl]pyrimidin-5-yl}pyridine-3-carboxylic acid

To a suspension of Example 630 (1 eq) taken in acetonitrile and water (1:1), was added sodium hydroxide (4 eq) and the mixture was stirred at RT for 1 h. The reaction mixture was concentrated in vacuo, acidified with 1 N HCl and the precipitate formed was filtered, washed and dried to give the title compound.

### Example 643

### 5-(2-{[3-(methylsulfonyl)phenyl]amino}-4-[3-(trifluoromethyl)-1H-pyrazol-1-yl]pyrimidin-5-yl)pyridine-3-carboxylic acid

To a suspension of Example 633 (1 eq) taken in dioxane and water (2:1), was added Barium hydroxide monohydrate (2 eq) and the mixture was allowed to stir overnight at RT. The reaction mixture was concentrated in vacuo, acidified with 1 N HCl and the precipitate formed was filtered, washed with water and dried to give the title compound.

### Example 644

### 5-(2-{[3-(methylsulfonyl)phenyl]amino}-4-[5-methyl-3-(trifluoromethyl)-1H-pyrazol-1-yl]pyrimidin-5-yl)pyridine-3-carboxylic acid

To a suspension of Example 634 (1 eq) taken in dioxane and water (2:1), was added Barium hydroxide monohydrate (2 eq) and the mixture was allowed to stir overnight at RT. The reaction mixture was concentrated in vacuo, acidified with 1 N HCl and the precipitate formed was filtered, washed with water and dried to give the title compound.

| Compound | Structure | Mass spectrum and ¹H NMR | SM |
|---|---|---|---|
| Example 635 | 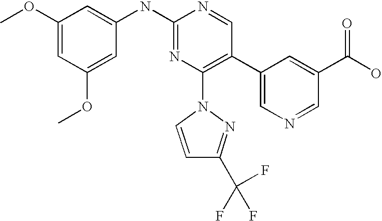 5-{2-[(3,5- dimethoxyphenyl) amino]-4-[3- (trifluoromethyl)-1H- pyrazol-1- yl]pyrimidin-5- yl}pyridine-3- carboxylic acid | MS(ES): 487 (M + 1) for C₂₂H₁₇F₃N₆O₄. 400 MHz, DMSO-d₆: δ 3.74 (s, 6H), 6.20 (s, 1H), 7.00 (d, J = 2.04 Hz, 1H), 7.10 (d, J = 1.72 Hz, 2H), 7.98 (s, 1H), 8.39 (br s, 1H), 8.43 (br s, 1H), 8.77 (s, 1H), 8.95 (s, 1H), 10.20 (s, 1H). | Example 625 ethyl 5-{2- [(3,5- dimethoxy- phenyl)amino]- 4-[3- (trifluoromethyl)- 1H- pyrazol-1- yl]pyrimidin- 5- yl}pyridine- 3-carboxylate |
| Example 636 | 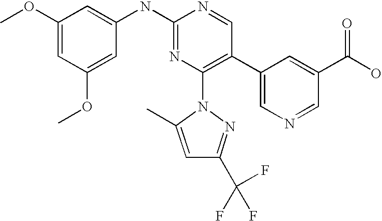 5-{2-[(3,5- dimethoxyphenyl) amino]-4-[5-methyl-3- (trifluoromethyl)-1H- pyrazol-1- yl]pyrimidin-5- yl}pyridin-3- carboxylic acid | MS(ES): 501 (M + 1) for C₂₃H₁₉F₃N₆O₄. 400 MHz, DMSO-d₆: δ 2.40 (s, 3 H), 3.72 (s, 6H), 6.22 (s, 1H), 6.76 (s, 1H), 7.04 (d, J = 1.76 Hz, 2H), 7.82 (d, J = 2.01 Hz, 1H), 8.54 (d, J = 1.76 Hz, 1H), 8.95 (d, J = 1.25 Hz, 1H), 8.96 (s,1H), 10.24 (s, 1H), 13.43 (br s, 1H). | Example 626 ethyl 5-{2- [(3,5- dimethoxy phenyl)amino]- 4-[5-methyl- 3- (trifluoro- methyl)-1H- pyrazol-1- yl]pyrimidin- 5-yl} pyridine-3- carboxylate |
| Example 637 | 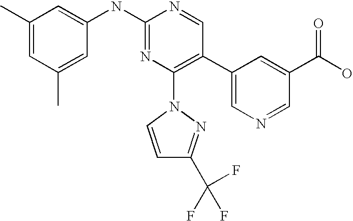 5-{2-[(3,5- dimethylphenyl) amino]-4-[3- (trifluoromethyl)-1H- pyrazol-1- yl]pyrimidin-5- yl}pyridine-3- carboxylic acid | MS(ES): 455 (M + 1) for C₂₂H₁₇F₃N₆O₂. 400 MHz, DMSO-d₆: δ 2.27 (s, 6H), 6.69 (s, 1H), 7.03 (dd, J = 2.64 Hz, 1H), 7.41 (s, 2H), 8.01 (t, J = 2.04 Hz, 1H), 8.52 (d, J = 1.68 Hz, 1H), 8.64 (d, J = 2.20 Hz, 1H), 8.77 (s, 1H), 8.99 (d, J = 1.92 Hz, 1H), 10.12 (s, 1H). | Example 627 ethyl 5-{2- [(3,5- dimethylphenyl) amino]-4- [3- (trifluoro- methyl)-1H- pyrazol-1- yl]pyrimidin- 5- yl}pyridine- 3-carboxylate |
| Example 638 | 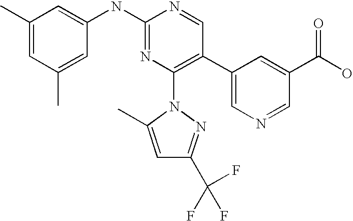 5-{2-[(3,5- dimethylphenyl) amino]-4-[5-methyl-3- (trifluoromethyl)-1H- pyrazol-1- yl]pyrimidin-5- yl}pyridine-3- carboxylic acid | MS(ES): 469 (M + 1) for C₂₃H₁₉F₃N₆O₂. 400 MHz, DMSO-d₆: δ 2.26 (s, 6H), 2.45 (s, 3H), 6.70 (s, 1H), 6.76 (s, 1H), 7.36 (s, 2H), 7.81 (s, 1H), 8.53 (s, 1H), 8.93-8.94 (m, 2H), 10.10 (s, 1H), 13.50 (s, 1H). | Example 628 ethyl 5-{2- [(3,5- dimethylphenyl) amino]-4- [5-methyl-3- (trifluoro- methyl)-1H- pyrazol-1- yl]pyrimidin- 5- yl}pyridine- 3-carboxylate |
| Example 639 | 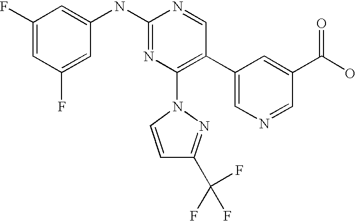 5-{2-[(3,5- difluorophenyl)amino]- 4-[3- (trifluoromethyl)-1H- pyrazol-1- yl]pyrimidin-5- yl}pyridine-3- carboxylic acid | MS(ES): 463 (M + 1) for C₂₀H₁₁F₅N₆O₂. 400 MHz, DMSO-d₆: δ 6.86 (tt, J = 2.10, 9.19 Hz, 1H), 7.04 (d, J = 2.44 Hz, 2H), 7.58 (d, J = 8.24 Hz, 1H), 7.99 (s, 1H), 8.48 (br s, 1H), 8.49 (br s, 1H), 8.86 (s, 1H), 8.96 (s, 1H), 10.67 (s, 1H). | Example 631 5-{2-[(3,5- difluorophenyl) amino]-4- [3- (trifluoro- methyl)-1H- pyrazol-1- yl]pyrimidin- 5- yl}pyridine- 3-carboxylate |
| Example 640 | 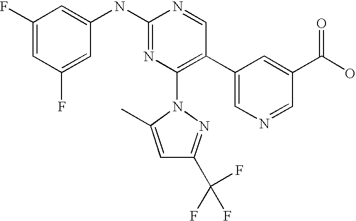 5-{2-[(3,5- difluorophenyl)amino]- 4-[5-methyl-3- (trifluoromethyl)-1H- pyrazol-1- yl]pyrimidin-5- yl}pyridine-3- carboxylic acid | MS(ES): 477 (M + 1) for C₂₁H₁₃F₅N₆O₂. 400 MHz, DMSO-d₆: δ 2.42 (s, 3H), 6.78 (s, 1H), 6.88 (t, J = 9.12 Hz, 1H), 7.54 (d, J = 8.68 Hz, 2H), 7.84 (s, 1H), 8.56 (br s, 1H), 8.98 (br s, 1H), 9.05 (s, 1H), 10.69 (s, 1H), 13.20 (br s, 1H). | Example 632 ethyl 5-{2- [(3,5- difluorophenyl) amino]-4- [5-methyl-3- (trifluoro- methyl)-1H- pyrazol-1- yl]pyrimidin- 5- yl}pyridine- 3-carboxylate |
| Example 641 | 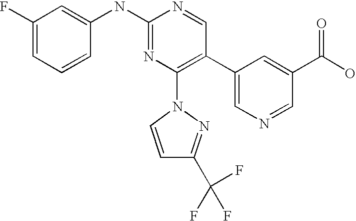 5-{2-[(3- fluorophenyl)amino]- 4-[3- (trifluoromethyl)-1H- pyrazol-1- yl]pyrimidin-5- yl}pyridine-3- carboxylic acid | MS(ES): 445 (M + 1) for C₂₀H₁₂F₄N₆O₂. 400 MHz, DMSO-d₆: δ 6.86 (td, J = 2.51, 8.41 Hz, 1H), 7.05 (d, J = 2.76 Hz, 1H), 7.34-7.44 (m, 1H), 7.56 (dd, J = 1.38, 8.16 Hz, 1H), 7.77 (dt, J = 1.98, 12.11, Hz, 1H), 8.02 (t, J = 2.01 Hz, 1H), 8.57 (d, J = 1.51 Hz, 1H), 8.65 (d, J = 2.01 Hz, 1H), 8.84 (s, 1H), 9.00 (d, J = 2.01 Hz, 1H), 10.49 (s, 1H), 13.43 (br s, 1H). | Example 629 ethyl 5-{2- [(3- fluorophenyl) amino]-4-[3- (trifluoro- methyl)-1H- pyrazol-1- yl]pyrimidin- 5- yl}pyridine- 3-carboxylate |
| Example 642 | 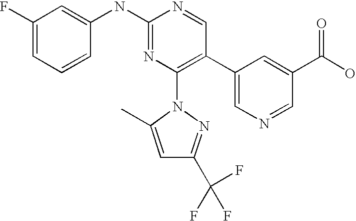 5-{2-[(3- fluorophenyl)amino]- 4-[5-methyl-3- (trifluoromethyl)-1H- pyrazol-1- yl]pyrimidin-5- yl}pyridine-3- carboxylic acid | MS(ES): 459 (M + 1) for C₂₁H₁₄F₄N₆O₂. 400 MHz, DMSO-d₆: δ 2.42 (s, 3H), 6.77 (s, 1H), 6.86 (td, J = 2.13, 8.38 Hz, 1H), 7.34-7.42 (m, 1H), 7.52 (d, J = 8.22 Hz, 1H), 7.76 (d, J = 11.57 Hz, 1H), 7.83 (s, 1H), 8.52-8.57 (m, 1H), 8.96 (s, 1H), 9.01 (s, 1H), 10.52 (s, 1H), 13.42 (br s, 1H). | Example 630 ethyl 5-{2- [(3- fluorophenyl) amino]-4-[5- methyl-3- (trifluoro- methyl)-1H- pyrazol-1- yl]pyrimidin- 5- yl}pyridine- 3-carboxylate |
| Example 643 | 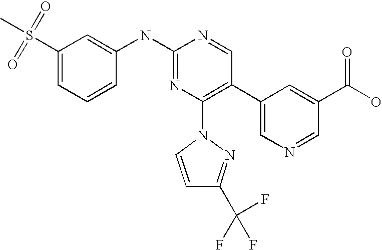 5-(2-{[3- (methylsulfonyl) phenyl]amino}-4-[3- (trifluoromethyl)-1H- pyrazol-1- yl]pyrimidin-5- yl)pyridine-3- carboxylic acid | MS(ES): 505 (M + 1) for C₂₁H₁₅F₃N₆O₄S. 400 MHz, DMSO-d₆: δ 3.24 (s, 3H), 7.06 (d, J = 2.75 Hz, 1H), 7.53-7.76 (m, 2H), 7.98 (d, J = 7.93 Hz, 1H), 8.08 (s, 1H), 8.68 (s, 2H), 8.63 (s, 1H), 8.85 (s, 1H), 9.02 (s, 1H), 10.69 (s, 1H). | Example 633 ethyl 5-(2- {[3- (methyl- sulfonyl) phenyl]-amino}-4-[3- (trifluoro- methyl)-1H- pyrazol-1- yl]pyrimidin- 5-yl)pyridine- 3-carboxylate |
| Example 644 | 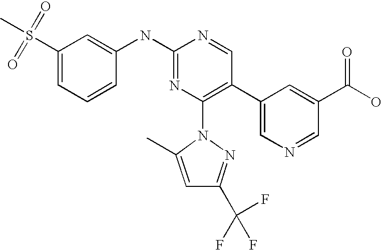 5-(2-{[3- (methylsulfonyl) phenyl]amino}-4-[5- methyl-3- (trifluoromethyl)-1H- pyrazol-1- yl]pyrimidin-5- yl)pyridine-3- carboxylic acid | MS(ES): 519 (M + 1) for C₂₂H₁₇F₃N₆O₄S. 400 MHz, CD₃COOD: δ 2.63 (br s, 3H), 3.16 (s, 3H), 6.62 (s, 1H), 7.60-7.67 (m, 1H), 7.69-7.76 (m, 1H), 7.95 (d, J = 7.78 Hz, 1H), 8.26 (br s, 1H), 8.62 (br s, 2H), 8.87 (br s, 1H), 9.22 (br s, 1H). | Example 634 ethyl 5-(2- {[3- (methyl- sulfonyl)- phenyl] amino}-4-[5- methyl-3- (trifluoro- methyl)-1H- pyrazol-1- yl]pyrimidin- 5-yl)pyridine- 3-carboxylate |

### Example 645

### methyl 5-{2-[(3,5-dimethylphenyl)amino]-4-[5-methyl-3-(trifluoromethyl)-1H-pyrazol-1-yl]pyrimidin-5-yl}-2-methoxypyridine-3-carboxylate

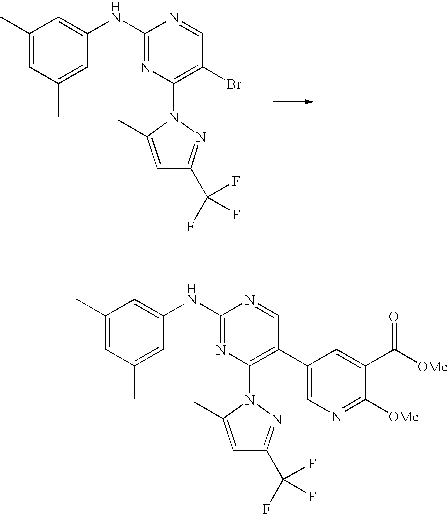

A suspension of 5-bromo-N-(3,5-dimethylphenyl)-4-[5-methyl-3-(trifluoromethyl)-1H-pyrazol-1-yl]pyrimidin-2-amine (Intermediate 218, 0.59 mmol, 250 mg), methyl 2-methoxy-5-(4,4,5,5-tetramethyl-1,3,2-dioxaborolan-2-yl)pyridine-3-carboxylate (0.58 mmol, 171 mg), [1,1′-bis(diphenylphosphino)ferrocene]dichloropalladium(II) complex with dichloromethane (0.099 mmol, 81 mg) and sodium carbonate (0.58 mmol, 61 mg) were taken in a mixture of acetonitrile and water (20 mL:5 mL) and heated to 90° C. for 10-20′. Acetonitrile was concentrated in vacuo. The residue was taken in ethyl acetate, washed with water and brine, dried over sodium sulphate, filtered and concentrated. The crude mass was purified by silica gel column chromatography (230-400 mesh) using 20% ethyl acetate/hexanes to yield 150 mg of the product.

| | | Mass spectram and ¹H | |
|---|---|---|---|
| Compound | Structure | NMR | SM |
| Example 645 | 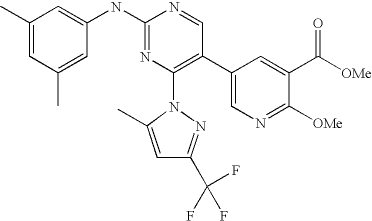 methyl 5-{2-[(3,5- dimethylphenyl)amino]- 4-[5-methyl-3- (trifluoromethyl)-1H- pyrazol-1-yl]pyrimnidin- 5-yl}-2- methoxypyridine-3- carboxylate | MS(ES): 513 (M + 1) for C₂₅H₂₃F₃N₆O₃. 300 MHz, DMSO-d₆: δ 2.24 (s, 6H), 2.33 (s, 3H), 3.74 (s, 3H), 3.90 (s, 3H), 6.68 (s, 1H), 6.74 (s, 1H), 7.35 (s, 2H), 7.58 (d, J = 2.49 Hz, 1H), 8.21 (d, J = 2.49 Hz, 1H), 8.89 (s, 1H), 10.09 (s, 1H). | Intermediate 218 5-bromo-N- (3,5- dimethyl- phenyl)-4-[5- methyl-3- (trifluoro- methyl)-1H- pyrazol-1- yl]pyrimidin- 2-amine |

### Example 646

### 5-{2-[(3,5-dimethylphenyl)amino]-4-∂5-methyl-3-(trifluoromethyl)-1H-pyrazol-1-yl]pyrimidin-5-yl}-2-methoxypyridine-3-carboxylic acid

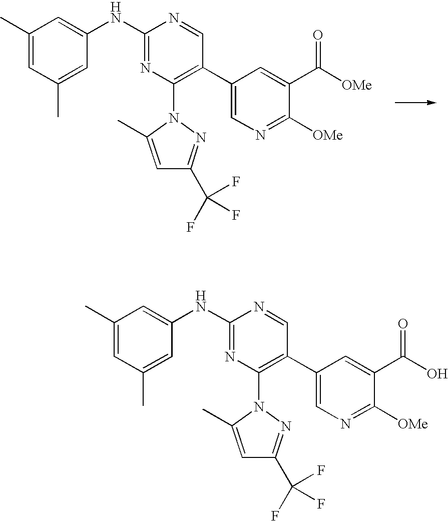

To a suspension of methyl 5-{2-[(3,5-dimethylphenyl)amino]-4-[5-methyl-3-(trifluoromethyl)-1H-pyrazol-1-yl]pyrimidin-5-yl}-2-methoxypyridine-3-carboxylate (Example 645, 0.29 mmol, 0.15 g) in THF and water (1:1), NaOH (0.58 mmol, 23 mg) was added and the mixture was stirred for 4 hours at rt. The THF was removed in vacuo and the reaction mixture diluted with water, acidified to pH=2 using 1.5 N HCl and the solid that precipitated was filtered and dried to get 0.065 g of the title compound.

| | | Mass spectrum and ¹H | |
|---|---|---|---|
| Compound | Structure | NMR | SM |
| Example 646 | 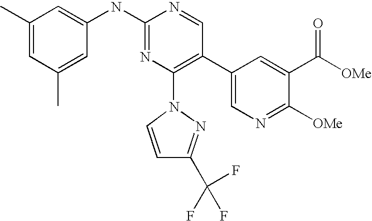 5-{2-[(3,5- dimethylphenyl)amino]-4- [5-methyl-3- (trifluoromethyl)-1H- pyrazol-1-yl]pyrimidin-5- yl}-2-methoxypyridine-3- carboxylic acid | MS(ES): 499 (M + 1) for C₂₄H₂₁F₃N₆O₃ . 400 MHz, DMSO-d₆: δ 2.26 (s, 6H), 2.36 (s, 3H), 3.90 (s, 3H), 6.70 (s, 1H), 6.75 (s, 1H), 7.36 (s, 2H), 7.61 (d, J = 2.40 Hz, 1H), 8.15 (d, J = 2.24 Hz, 1H), 8.90 (s, 1H), 10.10 (s, 1H), 12.9 (br s, 1H). | Example 645 methyl 5-{2- [(3,5- dimethylphenyl) amino]-4-[5- methyl-3- (trifluoromethyl- 1H-pyrazol-1- yl]pyrimidin-5- yl}-2- methoxypyridine- 3-carboxylate |

### Example 647

### methyl 5-{2-[(3,5-dimethylphenyl)amino]-4-[3-(trifluoromethyl)-1H-pyrazol-1-yl]pyrimidin-5-yl}-2-methoxypyridine-3-carboxylate

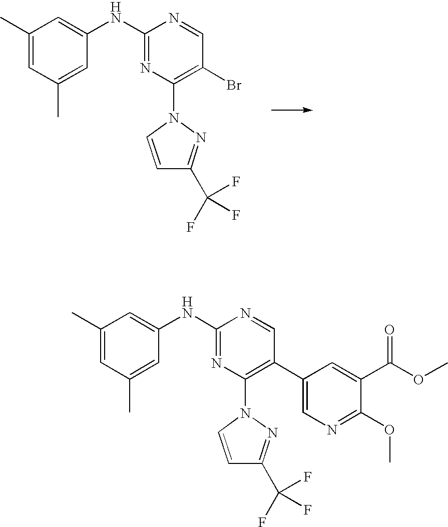

A solution of 5-bromo-N-(3,5-dimethylphenyl)-4-[3-(trifluoromethyl)-1H-pyrazol-1-yl]pyrimidin-2-amine (Intermediate 217, 0.8 mmol, 330 mg), methyl 2-methoxy-5-(4,4,5,5-tetramethyl-1,3,2-dioxaborolan-2-yl)pyridine-3-carboxylate (0.8 mmol, 234 mg), [1,1′-bis(diphenylphosphino)ferrocene]dichloropalladium(II) complex with CH₂Cl₂ (0.16 mmol, 130 mg) and sodium carbonate (0.8 mmol, 85 mg) in acetonitrile (16 mL)/water (4 mL) was degassed and heated to 90° C. for 20′ under nitrogen. The reaction mixture was concentrated in vacuo. The residue obtained was taken in ethyl acetate, washed with water and brine, dried over sodium sulfate and concentrated. The crude mass was purified by silica gel column chromatography (60-120 mesh) using 15% ethyl acetate/hexanes to yield 150 mg of methyl 5-{2-[(3,5-dimethylphenyl)amino]-4-[3-(trifluoromethyl)-1H-pyrazol-1-yl]pyrimidin-5-yl}-2-methoxypyridine-3-carboxylate.

| Compound | Structure | Mass spectrum and ¹H NMR | SM |
|---|---|---|---|
| Example 647 | 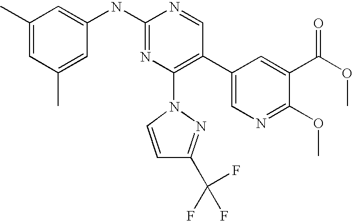 methyl 5-{2-[(3,5- dimethylphenyl) amino]-4-[3- (trifluoromethyl)- 1H-pyrazol-1- yl]pyrimidin-5- yl}-2- methoxypyridine- 3-carboxylate | MS(ES): 499 (M + 1) for C₂₄H₂₁F₃N₆O₃. 300 MHz, DMSO-d₆: δ 2.26 (s, 6H), 3.75 (s, 3H), 3.93 (s, 3H), 6.68 (s, 1H), 7.02 (d, J = 2.64 Hz, 1H), 7.40 (s, 3H), 7.84 (d, J = 2.46 Hz, 1H), 8.26 (d, J = 2.49 Hz, 1H), 8.48 (d, J= 1.68 Hz, 1H), 8.72 (s, 1H), 10.07 (s, 1H). | Intermediate 217 5-bromo-N- (3,5- dimethyl- phenyl)-4-[3- (trifluoro- methyl)-1H- pyrazol-1- yl]pyrimidin- 2-amine |

### Example 648

### 5-{2-[(3,5-dimethylphenyl)amino]-4-[3-(trifluoromethyl)-1H-pyrazol-1-yl]pyrimidin-5-yl}-2-methoxypyridine-3-carboxylic acid

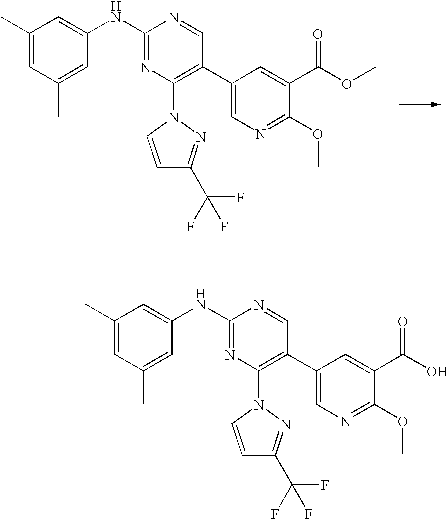

A solution of methyl 5-{2-[(3,5-dimethylphenyl)amino]-4-[3-(trifluoromethyl)-1H-pyrazol-1-yl]pyrimidin-5-yl}-2-methoxypyridine-3-carboxylate (Example 647, 0.3 mmol, 150 mg) and sodium hydroxide (0.6 mmol, 24 mg) in THF (3 mL) and water (3 mL) was stirred at room temperature for 3 h. THF was removed in vacuo and the reaction mixture was neutralized using 1.5 N HCl. The solid that precipitated out was filtered, washed with water and dried to give the title compound (65 mg).

| Compound | Structure | Mass spectrum and ¹H NMR | SM |
|---|---|---|---|
| Example 648 | 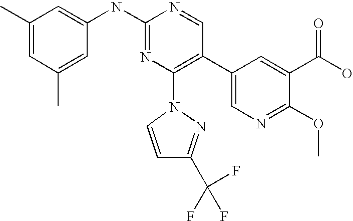 5-{2-[(3,5- dimthylphenyl) amino]-4-[3- (trifluoromethyl)- 1H-pyrazol-1- yl]pyrimidin-5- yl}-2- methoxypyridine- 3-carboxylic acid | MS(ES): 485 (M + 1) for C₂₃H₁₉F₃N₆O₃. 300 MHz, DMSO-d₆: δ 2.28 (s, 6H), 3.94 (s, 3H), 6.69 (s, 1H), 7.03 (d, J = 1.92 Hz, 1H), 7.42 (s, 2H), 7.82 (d, J = 1.83 Hz, 1H), 8.24 (d, J = 1.86 Hz, 1H), 8.48 (d, J = 1.29 Hz, 1H), 8.75 (s, 1H), 10.08 (s, 1H), 12.93 (br s, 1H). | Example 647 methyl 5-{2- [(3,5- dimethyl- phenyl) amino]-4- [3- (trifluoro- methyl)-1H- pyrazol-1- yl]pyrimidin- 5-yl}-2- methoxy- pyridine-3- carboxylate |

### General method for the synthesis of ethyl(2E)-3-(3-{2-[arylamino]-4-[3-(trifluoromethyl)-1H-pyrazol-1-yl]pyrimidin-5-yl}phenyl)prop-2-enoate, ethyl(2E)-3-(3-{2-[arylamino]-4-[5-methyl-3-(trifluoromethyl)-1H-pyrazol-1-yl]pyrimidin-5-yl}phenyl)prop-2-enoate and (2E)-3-[3-(2-{[3-methoxy-5-(methylsulfonyl)phenyl]amino}-4-[5-methyl-3-(trifluoromethyl)-1H-pyrazol-1-yl]pyrimidin-5-yl)phenyl]prop-2-enoic acid

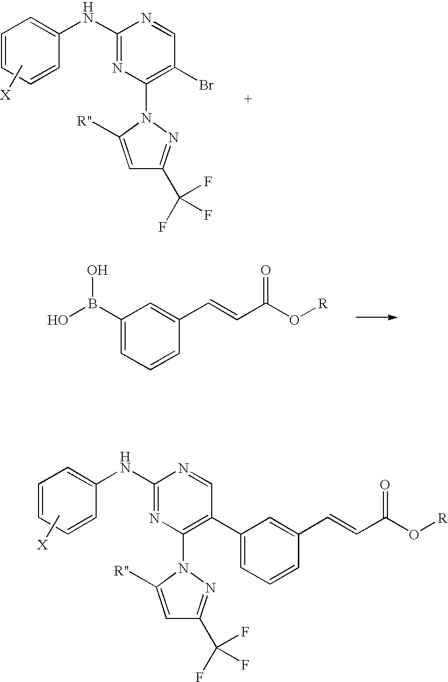

A suspension of 5-bromo-N-(aryl)-4-[3-(trifluoromethyl)-1H-pyrazol-1-yl]pyrimidin-2-amine derivative or 5-bromo-N-(aryl)-4-[5-methyl-3-(trifluoromethyl)-1H-pyrazol-1-yl]pyrimidin-2-amine derivative (1 eq), {3-[(1E)-3-ethoxy-3-oxoprop-1-en-1-yl]phenyl}boronic acid or 3-(trans-2-carboxyvinyl)phenylboronic acid (1.1-1.2 eq), [1,1′-bis(diphenylphosphino)ferrocene]dichloropalladium(II) (20 mol %) and sodium carbonate (1 eq) in acetonitrile/water (4:1) was degassed and heated to 90° C. for 15-30 min under inert atmosphere. The solvent was removed in vacuo and the crude mixture taken in CHCl₃ was washed with water, brine, dried over Na₂SO₄, filtered and concentrated. The crude material was purified by silica gel column chromatography using chloroform:methanol (9:1) as eluent to give the product. The compounds in the below table were prepared using the above method and the starting material specified.

| | | Mass spectrum and ¹H | |
|---|---|---|---|
| Compound | Structure | NMR | SM |
| Example 649^{h)} | 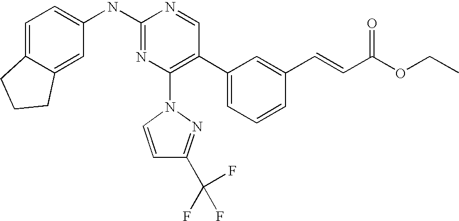 ethyl (2E)-3-(3-{2-(2,3- dihydro-1H-inden-5- ylamino)-4-[3- (trifluoromethyl)-1H- pyrazol-1-yl]pyrimidin- 5-yl}phenyl)prop-2- enoate | MS(ES): 520 (M + 1) for C₂₈H₂₄F₃N₅O₂. 300 MHz, DMSO-d₆: δ 1.24 (t, J = 7.08 Hz, 3H), 1.98-2.03 (m, 2H), 2.78- 2.87 (m, 4H), 4.17 (q, J = 7.08 Hz, 2H), 6.57 (d, J = 16.05 Hz, 1H), 6.97 (d, J = 2.25 Hz, 1H), 7.11 (d, J = 7.86 Hz, 1H), 7.17 (d, J = 8.20 Hz, 1H), 7.35 (t, J = 7.65 Hz, 1H), 7.48- 7.56 (m, 3H), 7.62 (d, J = 3.63 Hz, 1H), 7.66 (s, 1H), 8.35 (s, 1H), 8.74 (s, 1H), 10.10 (s, 1H). | Intermediate 242 5-bromo-N- (2,3-dihydro- 1H-inden-5- yl)-4-[3- (trifluoro- methyl)-1H- pyrazol-1- yl]pyrimidin- 2-amine |
| Example 650^{h)} | 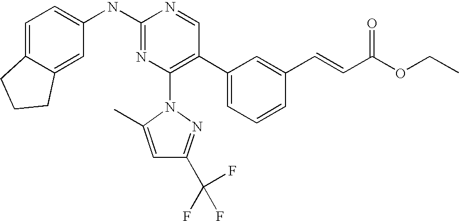 ethyl (2E)-3-(3-{2-(2,3- dihydro-1H-inden-5- ylamino)-4-[5-methyl-3- (trifluoromethyl)-1H- pyrazol-1-yl]pyrimidin- 5-yl}phenyl)prop-2- enoate | MS(ES): 534 (M + 1) for C₂₉H₂₆F₃N₅O₂. 400 MHz, DMSO-d₆: δ 1.26 (t, J =7.08 Hz, 3H), 2.01-2.04 (m, 2H), 2.21 (s,3H), 2.80-2.87 (m, 4H), 4.19 (q, J = 7.08 Hz, 2H), 6.55 (d, J = 16.04 Hz, 1H), 6.71 (s, 1H), 7.04 (d, J = 7.36 Hz, 1H), 7.18 (d, J = 8.08 Hz, 1H), 7.33-7.37 (m, 2H), 7.47 (d, J = 8.24 Hz, 1H), 7.56 (d, J = 16.00 Hz, 1H), 7.62-7.66 (m, 2H), 8.91 (s, 1H), 10.15 (s, 1H). | Intermediate 243 5-bromo-N- (2,3-dihydro- 1H-inden-5- yl)-4-[5- methyl-3- (trifluoro- methyl)-1H- pyrazol-1- yl]pyrimidin- 2-amine |
| Example 651^{h)} | 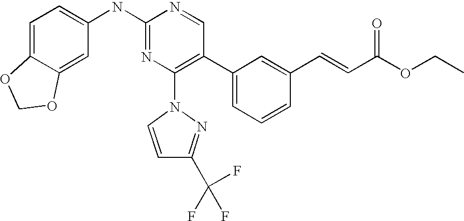 ethyl (2E)-3-(3-{2-(1,3- benzodioxol-5-ylamino)- 4-[3-(trifluoromethyl)- 1H-pyrazol-1- yl]pyrimidin-5- yl}phenyl)prop-2-enoate | MS(ES): 524 (M + 1) for C₂₆H₂₀F₃N₅O₄. 300 MHz, DMSO-d₆: δ 1.24 (t, J = 7.05 Hz, 3H), 4.17 (q, J = 7.02 Hz, 2H), 5.99 (s, 2H), 6.57 (d, J = 16.41 Hz, 1H), 6.89 (d, J = 8.34 Hz, 1H), 6.98 (s, 1H), 7.13 (t, J = 12.57 Hz, 2H), 7.35 (t, J = 8.40 Hz, 1H), 7.47 (s, 1H), 7.54 (d, J = 10.53 Hz, 1H), 7.63 (d, J = 11.88 Hz, 2H), 8.35 (s, 1H), 8.74 (s,1H),10.11 (s, 1H). | Intermediate 244 N-(1,3- benzodioxol- 5-yl)-5- bromo-4-[3- (trifluoro- methyl)-1H- pyrazol-1- yl]pyrimidin- 2-amine |
| Example 652^{h)} | 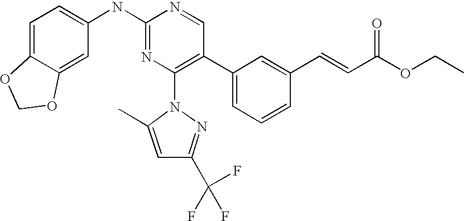 ethyl (2E)-3-(3-{2-(1,3- benzodioxol-5-ylamino)- 4-[5-methyl-3- (trifluoromethyl)-1H- pyrazol-1-yl]pyrimidin- 5-yl}phenyl)prop-2- enoate | MS(ES): 538 (M + 1) for C₂₇H₂₂F₃N₅O₄. 300 MHz, DMSO-d₆: δ 1.24 (t, J = 7.08 Hz, 3H), 2.17 (s, 3H), 4.17 (q, J = 7.11 Hz, 2H), 5.98 (s, 2H), 6.53 (d, J = 16.05 Hz, 1H), 6.69 (s, 1H), 6.89 (d, J = 8.40 Hz, 1H), 7.01 (d, J = 7.44 Hz, 1H), 7.11 (d, J = 8.49 Hz, 1H), 7.31 (s, 1H), 7.34 (d, J = 4.35 Hz, 1H), 7.41 (s, 1H), 7.54 (d, J = 15.99 Hz, 1H), 7.62 (d, J = 7.47 Hz, 1H), 8.88 (s, 1H), 10.14 (s, 1H). | Intermediate 245 benzodioxol- 5-yl)-5- bromo-4-(5- methyl-3- (trifluoro- methyl)-1H- pyrazol-1- yl]pyrimidin- 2-amine |
| Example 653ⁱ⁾ | 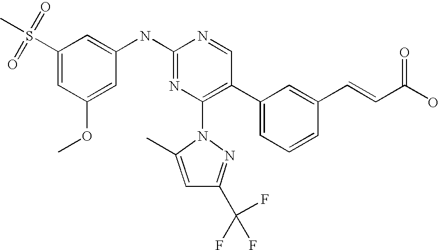 (2E)-3-[3-(3-{[3- methoxy-5- (methylsulfonyl)phenyl] amino}-4-[5-methyl-3- (trifluoromethyl)-1H- pyrazol-1-yl]pyrimidin- 5-yl)phenyl]prop-2-enoic acid | MS(ES): 574 (M + 1) for C₂₆H₂₂F₃N₅O₅S. 400 MHz, DMSO-d₆: δ 2.27 (s, 3H), 3.23 (s, 3H), 3.85 (s, 3H), 6.46 (d, J = 16.04 Hz, 1H), 6.75 (s, 1H), 7.05 (d, J = 7.76 Hz, 1H), 7.12 (s, 1H), 7.36- 7.39 (m, 2H), 7.50 (d, J = 15.88 Hz, 1H), 7.62 (d, J = 7.72 Hz, 1H), 7.76 (s, 1H), 8.02 (s, 1H), 9.03 (s, 1H), 10.64 (s, 1H), 12.44 (br s, 1H). | Intermediate 246 5-bromo-N- [3-methoxy- 5- (methyl- sulfonyl) phenyl]- 4-[5-methyl- 3- (trifluoro- methyl)-1H- pyrazol-1- yl]pyrimidin- 2-amine and 3-(trans-2- carboxyvinyl)- phenylboronic acid |

^{h)}{3-[(1E)-3-ethoxy-3-oxoprop-1-en-1-yl]phenyl}boronic acid was used
ⁱ⁾3-(trans-2-carboxyvinyl)phenylboronic acid (1.1 eq) and Na₂CO₃ (2 eq) were used

### Example 654

### ethyl(2E)-3-{3-[2-{[3-methoxy-5-(methylsulfonyl)phenyl]amino}-4-(methylsulfonyl)pyrimidin-5-yl]phenyl}prop-2-enoate

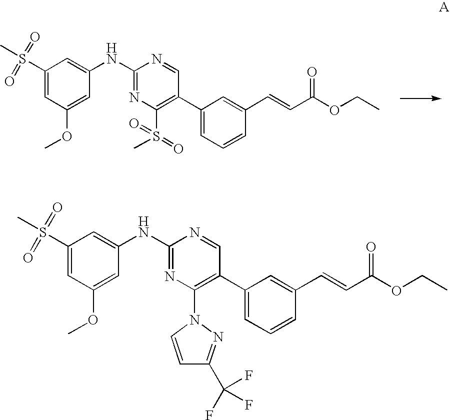

suspension of 3-(trifluoromethyl)-1H-pyrazole (1.2-1.5 eq), potassium tert-butoxide (1.5 eq) and ethyl(2E)-3-{3-[2-{[3-methoxy-5-(methylsulfonyl)phenyl]amino}-4-(methylsulfonyl)pyrimidin-5-yl]phenyl}prop-2-enoate (Intermediate 249, 1 eq) in DMSO (3 mL) was subjected to microwave irradiation at 130° C. for 1 h. After the reaction was cooled to RT, the mixture was diluted with EtOAc, washed successively with water and brine. The organic layer was dried over sodium sulphate, filtered, concentrated and further purified by silica gel column chromatography using ethyl acetate/hexanes to yield the title compound.

| | | Mass spectrum and | |
|---|---|---|---|
| Compound | Structure | ¹H NMR | SM |
| Example 654 | 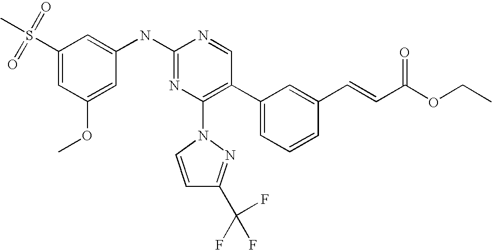 ethyl 92E)-3-{3-[2-{[3- methoxy-5- (methylsulfonyl)phenyl] amino}-4- (methylsulfonyl)pyrimidin- 5-yl]phenyl}prop-2- enoate | Taken to the next step based on LCMS without further purification. MS(ES): 588 (M + 1) for C₂₇H₂₄F₃N₅O₅S. (43% pure by LCMS). | Intermediate 249 ethyl (2E)-3-{3- [2-{[3-methoxy- 5- (methylsulfonyl) phenyl]amino}- 4- (methylsulfonyl) pyrimidin-5- yl]phenyl}prop- 2-enoate |

### General procedure for the synthesis of (2E)-3-(3-{2-(arylamino)-4-[-1H-pyrazol-1-yl]pyrimidin-5-yl}phenyl)prop-2-enoic acid

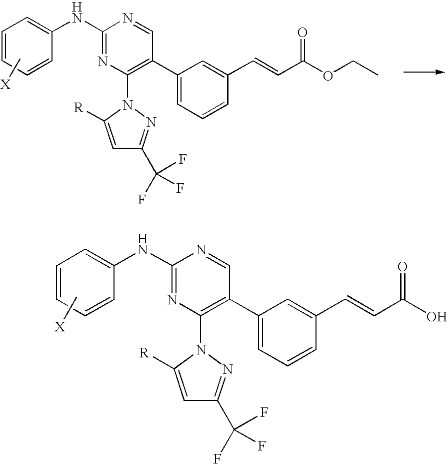

To 1 eq of the ethyl(2E)-3-(3-{2-[arylamino]-4-[1H-azol-1-yl]pyrimidin-5-yl}phenyl)prop-2-enoate derivative taken in dioxane (5 mL), was added Barium hydroxide (2-6 eqs) and was warmed to 60° C. for 1 to 2 h. The reaction mixture was carefully acidified with 1 N HCl and the solid that precipitated was filtered, washed with water and dried in vacuo to yield the desired carboxylic acid. The compounds in the below table were prepared using the above method and the starting material specified.

| | | Mass spectrum and ¹H | |
|---|---|---|---|
| Compound | Structure | NMR | SM |
| Example 655^{j)} | 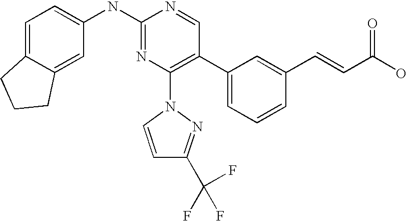 (2E)-3-(3-{2-(2,3- dihydro-1H-inden-5- ylamino)-4-[3- (trifluoromethyl)-1H- pyrazol-1-yl]pyrimidin- 5-yl}phenyl)prop-2-enoic acid | MS(ES): 492 (M + 1) for C₂₆H₂₀F₃N₅O₂ 400 MHz, DMSO-d₆: δ 1.99-22.06 (m, 2H), 2.81- 2.88 (m, 4H), 6.47 (d, J = 15.96 Hz, 1H), 6.97 (d, J = 2.32 Hz, 1H), 7.13 (d, J = 7.72 Hz, 1H), 7.18 (d, J = 8.08 Hz,1H), 7.36 (t, J = 7.72 Hz, 1H), 7.49-7.55 (m, 3H), 7.62 (d, J = 7.48 Hz, 1H), 7.68 (s, 1H), 8.36 (s, 1H), 8.75 (s,1H), 10.08 (s, 1H), 12.40 (s, 1H). | Example 649 ethyl (2E)-3- (3-{2-(2,3- dihydro-1H- inden-5- ylamino)-4- [3- (trifluoro- methyl)-1H- pyrazol-1- yl]pyrimidin- 5- yl}phenyl) prop-2-enoate |
| Example 656^{k)} | 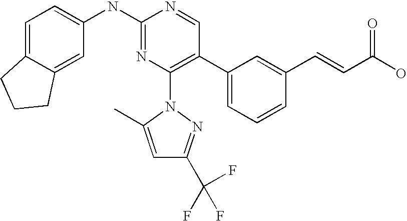 (2E)-3-(3-{2-(2,3- dihydro-1H-inden-5- ylamino)-4-[5-methyl-3- (trifluoromethyl)-1H- pyrazol-1-yl]pyrimidin- 5-yl}phenyl)prop-2-enoic acid | MS(ES): 506 (M + 1) for C₂₇H₂₂F₃N₅O₂ 400 MHz, DMSO-d₆: δ 1.98-2.05 (m, 2H), 2.20 (s, 3H), 2.80-2.86 (m, 4H), 6.40 (d, J = 16.04 Hz, 1H), 6.69 (s, 1H), 6.99 (d, J = 7.92 Hz, 1H), 7.17 (d, J = 8.08 Hz, 1H), 7.25 (s, 1H), 7.31 (t, J = 7.72 Hz, 1H, 7.35 (s, 1H), 7.46 (d, J = 7.40 Hz, 1H), 7.53 (d, J = 7.84 Hz, 1H), 7.64 (s,1H), 8.88 (s, 1H), 10.12 (s, 1H). | Example 650 ethyl (2E)-3- (3-{2-(2,3- dihydro-1H- inden-5- ylamino)-4- [5-methyl-3- (trifluoro- methyl)-1H- pyrazol-1- yl]pyrimidin- 5- yl}phenyl) prop-2-enoate |
| Example 657^{j)} | 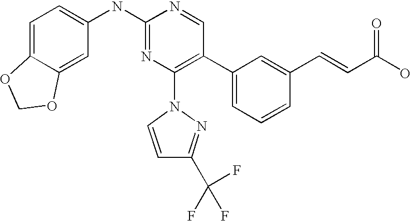 (2E)-3-(3-{2-(1,3- benzodioxol-5-ylamino)- 4-[3-(trifluoromethyl)- 1H-pyrazol-1- yl]pyrimidin-5- yl}phenyl)prop-2-enoic acid | MS(ES): 496 (M + 1) for C₂₄H₁₆F₃N₅O₄ 400 MHz, DMSO-d₆: δ 6.01 (s, 2H), 6.47 (d, J = 16.04 Hz, 1H), 6.91 (d, J = 8.36 Hz, 1H), 6.99 (d, J = 2.28 hz, 1H), 7.12 (d, J = 7.6 Hz, 1H), 7.17 (dd, J = 1.88, 8.32 Hz, 1H), 7.36 (t, J = 7.64 Hz, 1H), 7.48 (t, J = 2.08 Hz, 1H), 7.54 (d, J = 16.00 Hz, 1H), 7.63 (d, J = 7.56 Hz, 1H), 8.37 (s, 1H), 8.76 (s, 1H), 10.11 (s, 1H), 12.41 (br s, 1H). | Example 651 ethyl (2E)-3- (3-{2-(1,3- benzodioxol- 5-ylamino)- 4-[3- (trifluoro- methyl)-1H- pyrazol-1- yl]pyrimidin- 5- yl}phenyl) prop-2-enoate |
| Example 658^{k)} | 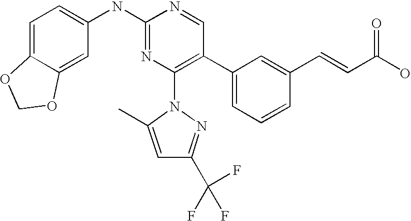 (2E)-3-(3-{2-(1,3- benzodioxol-5-ylamino)- 4-[5-methyl-3- (trifluoromethyl)-1H- pyrazol-1-yl]pyrimidin- 5-yl}phenyl)prop-2-enoic acid | MS(ES): 510 (M + 1) for C₂₅H₁₈F₃N₅O₄ 400 MHz, DMSO-d₆: δ 2.20 (s, 3H), 6.00 (s, 2H), 6.43 (d, J = 16.00 Hz, 1H), 6.70 (s, 1H), 6.90 (d, J = 8.40 Hz, 1H), 7.03 (d, J = 7.76 Hz, 1H), 7.12 (dd, J = 1.96, 8.46 Hz, 1H), 7.31 (br s, 1H), 7.35 (t, J = 7.76 Hz, 1H), 7.42 (br s, 1H), 7.49 (d, J = 15.96 Hz, 1H), 7.59 (d, J = 7.80 Hz, 1H), 8.89 (s, 1H), 10.13 (s, 1H), 12.41 (br s, 1H). | Example 652 ethyl (2E)-3- (3-{2-(1,3- benzodioxol- 5-ylamino)- 4-[5-methyl- 3- (trifluoro- methyl)-1H- pyrazol-1- yl]pyrimidin- 5- yl}phenyl) prop-2-enoate |
| Example 659^{j)} | 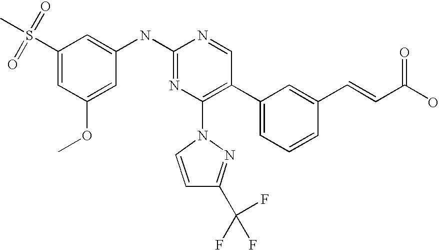 (2E)-3-[3-(2-{[3- methoxy-5- (methylsulfonyl)phenyl] amino}-4-[3- (trifluoromethyl)-1H- pyrazol-1-yl]pyrimidin- 5-yl)phenyl]prop-2-enoic acid | MS(ES): 560 (M + 1) for C₂₅H₂₀F₃N₅O₅S. 400 MHz, DMSO-d₆: δ 3.23 (s, 3H), 3.86 (s, 3H), 6.48 (d, J = 15.92 Hz, 1H), 7.01 (d, J = 2.52 Hz, 1H), 7.10 (s, 1H), 7.16 (d, J = 8.08 Hz, 1H), 7.37 (t, J = 7.64 Hz, 1H), 7.50-7.55 (m, 2H), 7.63 (d, J = 7.76 Hz, 1H), 7.75 (s, 1H), 8.12 (s, 1H), 8.41 (s, 1H), 8.85 (s, 1H), 10.60 (s, 1H), 12.30 (br s, 1H). | Example 654 ethyl 92E)-3- {3-[2-{[3- methoxy-5- (methyl- sulfonyl)- phenyl] amino}-4- (methyl- sulfonyl) pyrimidin-5- yl]phenyl} prop-2-enoate |

^{j)}Ba(OH)₂, dioxane-H₂O, RT, 12-24 h
^{k)}Ba(OH)₂, dioxane-H-₂O, 55° C., 1-2 h

### General procedure for the synthesis of ethyl 5-(2-{arylamino}-4-[1H-azol-1-yl]pyrimidin-5-yl)pyridine-3-carboxylate

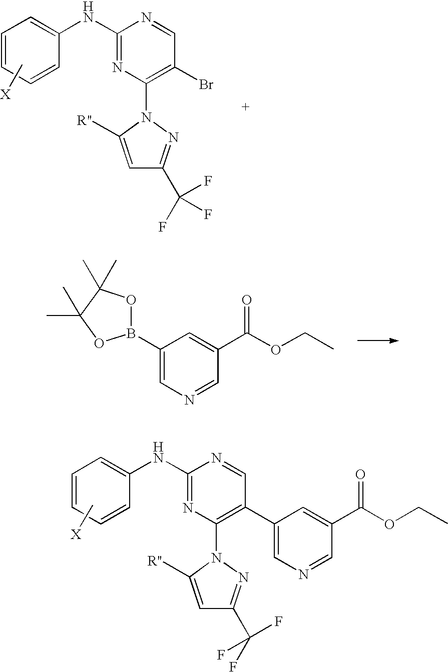

A suspension of 5-bromo-N-(aryl)-4-[3-(trifluoromethyl)-1H-pyrazol-1-yl]pyrimidin-2-amine or 5-bromo-N-(aryl)-4-[5-methyl-3-(trifluoromethyl)-1H-pyrazol-1-yl]pyrimidin-2-amine (1 eq), ethyl 5-(4,4,5,5-tetramethyl-1,3,2-dioxaborolan-2-yl)pyridine-3-carboxylate (1.1-1.2 eq), [1,1′-bis(diphenylphosphino)ferrocene]-dichloropalladium(II) (10-20 mol %) and sodium carbonate (1-2 eq) in acetonitrile/water (4:1) was degassed and heated to 90° C. for 15-30 min under inert atmosphere. The solvent was removed in vacuo and the crude mixture taken in CHCl₃ was washed with water, brine, dried over Na₂SO₄, filtered and concentrated. The crude material was purified by silica gel column chromatography using 30% ethyl acetate/hexanes as eluent to give the product. The compounds in the below table were prepared using this method and the specified starting material.

| | | Mass spectrum and ¹H | |
|---|---|---|---|
| Compound | Structure | NMR | SM |
| Example 660 | 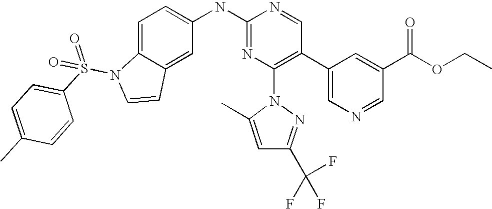 ethyl 5-{2-({1-[(4-methylphenyl)sulfonyl]-1H-indol-5-yl}amino)-4-[5-methyl- 3-(trifluoromethyl)-1H-pyrazol-1-yl]pyrimidin-5-yl}pyridine-3-carboxylate | MS(ES): 662 (M + 1) for C₃₂H₂₆F₃N₇O₄S. 300 MHz, DMSO-d₆: δ 1.30 (t, J = 7.08 Hz, 3H), 2.30 (s, 3H), 2.38 (s, 3H), 4.30 (q, J = 7.11 Hz, 2H), 6.75 (s, 1H), 6.82 (d, J = 3.60 Hz, 1H), 7.37 (d, J = 8.28 Hz, 2H), 7.60 (dd, J = 1.98, 9.06 Hz, 1H), 7.75- 7.76 (m, 2H), 7.84-7.91 (m, 3H), 7.99 (s, 1H), 8.58 (d, J = 2.13 Hz, 1H), 8.92 (s, 1H), 8.96 (d, J = 1.92 Hz, 1H), 10.33 (s, 1H). | Intermediate 241 N-{5-bromo- 4-[5-methyl- 3- (trifluoro- methyl)-1H- pyrazol-1- yl]pyrimidin- 2-yl}-1-[(4- methylphenyl) sulfonyl]- 1H-indol-5- amine |
| Example 661 | 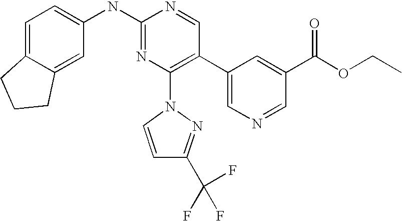 ethyl 5-{2-(2,3-dihydro-1H-inden-5-ylamino)-4-[3-(trifluoromethyl)-1H- pyrazol-1-yl]pyrimidin-5-yl}pyridine-3-carboxylate | MS(ES): 495 (M + 1) for C₂₅H₂₁F₃N₆O₂. 400 MHz, DMSO-d₆: δ 1.28-1.36 (m, 3H), 1.98- 2.04 (m, 2H), 2.80-2.88 (m, 4H), 4.30-4.35 (m, 2H), 7.02 (d, J = 2.44 Hz, 1H), 7.19 (d, J = 8.12 Hz, 1H), 7.50 (d, J = 8.12 Hz, 1H), 7.64 (s, 1H), 8.00 (s, 1H), 8.54 (s, 1H), 8.67 (d, J = 2.08 Hz, 1H), 8.76 (s, 1H), 9.00 (d, J = 1.84 Hz, 1H), 10.15 (s, 1H). | Intermediate 242 5-bromo-N- (2,3-dihydro- 1H-inden-5- yl)-4-[3- (trifluoro- methyl)-1H- pyrazol-1- yl]pyrimidin- 2-amine |
| Example 662 | 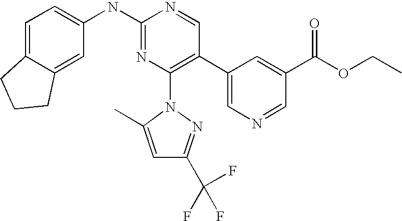 ethyl 5-{2-(2,3-dihydro-1H-inden-5-ylamino)-4-[5-methyl-3-(trifluoromethyl)- 1H-pyrazol-1-yl]pyrimidin-5-yl}pyridine-3-carboxylate | MS(ES): 509 (M + 1) for C₂₆H₂₃F₃N₆O₂. 400 MHz, DMSO-d₆: δ 1.31 (t, J = 7.2 Hz, 3H), 2.01-2.05 (m, 2H), 2.42 (s, 3H), 2.81-2.88 (m, 4H), 4.31 (q, J = 7.20 Hz, 2H), 6.77 (s, 1H), 7.20 (d, J = 8.00 Hz, 1H), 7.46 (d, J = 7.60 Hz, 1H), 7.63 (s, 1H), 7.78 (s, 1H), 8.60 (s, 1H), 8.94 (s, 1H), 8.97 (s, 1H), 10.20 (s, 1H). | Intermediate 243 5-bromo-N- (2,3-dihydro- 1H-inden-5- yl)-4-[5- methyl-3- (trifluoro- methyl)-1H- pyrazol-1- yl]pyrimidin- 2-amine |
| Example 663 | 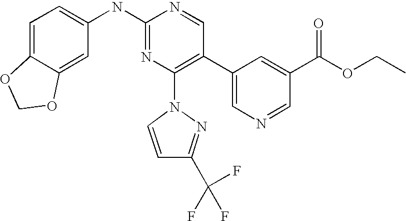 ethyl 5-{2-(1,3-benzodioxol-5-ylamino)-4-[3-(trifluoromethyl)-1H-pyrazol-1- yl]pyrimidin-5-yl}pyridine-3-carboxylate | Taken to the next step based on LCMS MS(ES): 499 (M + 1) C₂₃H₁₇F₃N₆O₄. | Intermediate 244 N-(1,3- benzodioxol- 5-yl)-5- bromo-4-[3- (trifluoro- methyl)-1H- pyrazol-1- yl]pyrimidin- 2-amine |
| Example 664 | 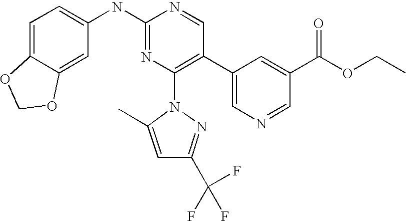 ethyl 5-{2-(1,3-benzodioxol-5-ylamino)-4-[5-methyl-3-(trifluoromethyl)-1H- pyrazol-1-yl]pyrimidin-5-yl}pyridine-3-carboxylate | MS(ES): 513 (M + 1) for C₂₄H₁₉F₃N₆O₄. 400 MHz, DMSO-d₆: δ 1.28 (t, J = 9.48 Hz, 3H), 2.37 (s, 3H), 4.30 (d, J = 9.92 Hz, 2H), 5.99 (s, 1H), 6.75 (s, 1H), 6.90 (d, J = 10.84 Hz, 1H), 7.09 (s, 1H), 7.47 (t, J = 46.68 Hz, 3H), 7.75 (s, 1H), 8.57 (s, 1H), 8.94 (d, J = 14.80 Hz, 1H), 10.19 (s, 1H). | Intermediate 244 N-(1,3- benzodioxol- 5-yl)-5- bromo-4-[5- methyl-3- (trifluoro- methyl)-1H- pyrazol-1- yl]pyrimidin- 2-amine |

### Example 665

### ethyl 5-(2-{[3-methoxy-5-(methylsulfonyl)phenyl]amino}-4-[3-(trifluoromethyl)-1H-pyrazol-1-yl]pyrimidin-5-yl)pyridine-3-carboxylate

### Example 666

### ethyl 5-(2-{[3-methoxy-5-(methylsulfonyl)phenyl]amino}-4-[5-methyl-3-(trifluoromethyl)-1H-pyrazol-1-yl]pyrimidin-5-yl)pyridine-3-carboxylate

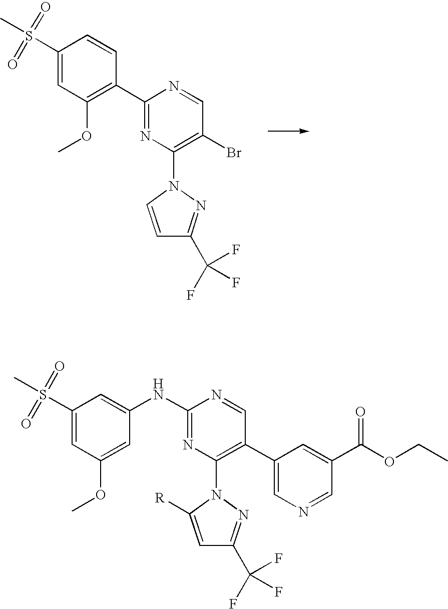

A suspension of 5-bromo-N-[3-methoxy-5-(methylsulfonyl)phenyl]-4-[3-(trifluoromethyl)-1H-pyrazol-1-yl]pyrimidin-2-amine (Intermediate 250) or 5-bromo-N-[3-methoxy-5-(methylsulfonyl)phenyl]-4-[5-methyl-3-(trifluoromethyl)-1H-pyrazol-1-yl]pyrimidin-2-amine (Intermediate 251) (1 eq), ethyl 5-(4,4,5,5-tetramethyl-1,3,2-dioxaborolan-2-yl)pyridine-3-carboxylate (1.1-1.2 eq), [1,1′-bis(diphenylphosphino)ferrocene]-dichloropalladium(II) (10-20 mol %) and sodium carbonate (1-2 eq) in acetonitrile/water (4:1) was degassed and heated to 90° C. for 15-30 min under inert atmosphere. The solvent was removed in vacuo and the crude mixture taken in CHCl₃ was washed with water, brine, dried over Na₂SO₄, filtered and concentrated. The crude material was purified by silica gel column chromatography using 50% ethyl acetate/hexanes as eluent to give the product. The compounds in the below table were prepared following this procedure and using the specified starting material.

| Compound | Structure | Mass spectrum and ¹H NMR | SM |
|---|---|---|---|
| Example 665 | 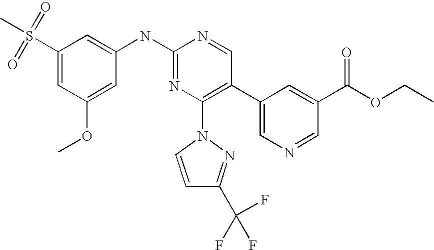 ethyl 5-(2-{[3-methoxy-5-(methylsulfonyl)phenyl]amino}-4-[3- (trifluoromethyl)-1H-pyrazol-1-yl]pyrimidin-5-yl)pyridine-3-carboxylate | MS(ES): 563 (M + 1) for C₂₄H₂₁F₃N₆O₅S. 400 MHz, DMSO-d₆: δ 1.31 (t, J = 7.08 Hz, 3H), 3.24 (s, 3H), 3.87 (s, 3H), 4.33 (q, J = 7.08 Hz, 2H), 7.08 (d, J = 2.56 Hz, 1H), 7.14 (s, 1H), 7.67 (s, 1H), 8.09 (t, J = 1.96 Hz, 1H), 8.19 (s, 1H), 8.62 (s, 1H), 8.72 (d, J = 2.16 Hz, 1H), 8.87 (s, 1H), 9.04 (d, J = 1.92 Hz, 1H), 10.66 (s, 1H). | Intermediate 250 5-bromo-N-[3- methoxy-5- (methylsulfonyl) phenyl]-4-[3- (trifluoromethyl)- 1H-pyrazol-1- yl]pyrimidin-2- amine |
| Example 666 | 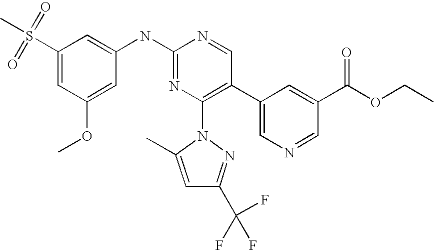 ethyl 5-(2-{[3-methoxy-5-(methylsulfonyl)phenyl]amino}-4-[5- methyl-3-(trifluoromethyl)-1H-pyrazol-1-yl]pyrimidin-5-yl)pyridine-3- carboxylate | Taken to the next step based on LCMS without further purification. MS(ES): 577 (M + 1) for C₂₅H₂₃F₃N₆O₅S. (88% pure by LCMS). | Intermediate 251 5-bromo-N-[3- methoxy-5- (methylsulfonyl) phenyl]-4-[5-methyl- 3- (trifluoromethyl)- 1H-pyrazol-1- yl]pyrimidin-2- amine |

### General procedure for the synthesis of 5-{2-(arylamino)-4-[1H-pyrazol-1-yl]pyrimidin-5-yl}pyridine-3-carboxylic acid

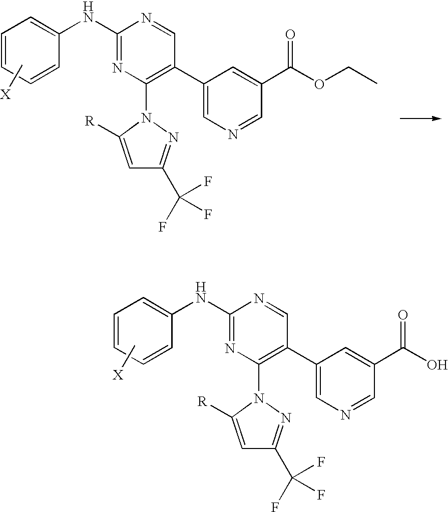

To 1 eq of the ethyl 5-(2-{arylamino}-4-[1H-azol-1-yl]pyrimidin-5-yl)pyridine-3-carboxylate derivative taken in dioxane (5 mL), was added 1 N aq. sodium hydroxide/Barium hydroxide (2-6 eqs) and was heated to 60° C. for 1 h. The reaction mixture was carefully acidified with 1 N HCl and the solid that precipitated was filtered, washed with water and dried in vacuo to yield the desired carboxylic acid. The compounds in the below table were prepared following this procedure and using the specified starting material.

| | | Mass spectrum and ¹H | |
|---|---|---|---|
| Compound | Structure | NMR | SM |
| Example 667^{l)} | 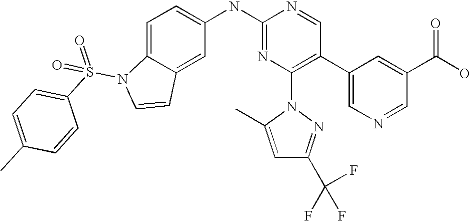 5-{2-({1-[(4-methylphenyl)sulfonyl]-1H-indol-5-yl}amino)-4-[5-methyl-3- (trifluoromethyl)-1H-pyrazol-1-yl]pyrimidin-5-yl}pyridine-3-carboxylic acid | MS(ES): 634 (M + 1) for C₃₀H₂₂F₃N₇O₄S. 400 MHz, DMSO-d₆: δ 2.31 (s, 3H), 2.34 (s, 3H), 6.72 (s, 1H), 6.83 (d, J = 3.56 Hz, 1H), 7.38 (d, J = 8.24 Hz, 2H), 7.61 (dd, J = 1.76, 5.46 Hz, 1H), 7.76 (d, J = 3.60 Hz, 1H), 7.83-7.90 (m, 4H), 8.00 (s, 1H), 8.28 (s, 1H), 8.88 (s, 1H), 8.88 (s, 1H), 10.30 (s, 1H). | Example 660 ethyl 5-{2- ({1-[(4- methylphenyl) sulfonyl]- 1H-indol-5- yl}amino)-4- [5-methyl-3- (trifluoro- methyl)-1H- pyrazol-1- yl]pyrimidin- 5- yl}pyridine- 3-carboxylate |
| Example 668ⁿ⁾ | 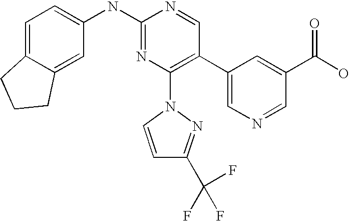 5-{2-(2,3-dihydro-1H-inden-5-ylamino)-4-[3-(trifluoromethyl)-1H-pyrazol- 1-yl]pyrimidin-5-yl}pyridine-3-carboxylic acid | MS(ES): 467 (M + 1) for C₂₃H₁₇F₃N₆O₂ 400 MHz, DMSO-d₆: δ 2.00-2.04 (m, 2H), 2.80- 2.88 (m, 4H), 6.98 (d, J = 2.60 Hz, 1H), 7.18 (d, J = 8.16 Hz, 1H), 7.50 (dd, J = 4.00, 10.00 Hz, 1H), 7.66 (s, 1H), 8.01 (s, 1H), 8.24 (d, J = 2.20 Hz, 1H), 8.44 (s, 1H), 8.70 (s, 1H), 8.95 (d, J = 1.72 Hz, 1H), 10.10 (s, 1H). | Example 661 ethyl 5-{2- (2,3-dihydro- 1H-inden-5- ylamino)-4- [3- (trifluoro- methyl)-1H- pyrazol-1- yl]pyrimidin- 5- yl}pyridine- 3-carboxylate |
| Example 669^{o)} | 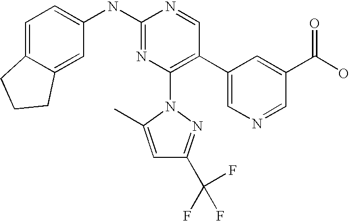 5-{2-(2,3-dihydro-1H-inden-5-ylamino)-4-[5-methyl-3-(trifluoromethyl)-1H- pyrazol-1-yl]pyrimidin-5-yl}pyridine-3-carboxylic acid | MS(ES): 481 (M + 1) for C₂₄H₁₉F₃N₆O₂ 400 MHz, DMSO-d₆: δ 2.01-2.04 (m, 2H), 2.34 (s, 3H), 2.80-2.87 (m, 4H), 6.72 (s, 1H), 7.18 (d, J = 8.16 Hz, 1H), 7.46 (d, J = 7.72 Hz, 1H), 7.63 (s, 1H), 7.89 (s, 1H), 8.12 (s, 1H), 8.84 (s, 1H), 8.88 (s, 1H), 10.14 (s, 1H). | Example 662 ethyl 5-{2- (2,3-dihydro- 1H-inden-5- ylamino)-4- [5-methyl-3- (trifluoro- methyl)-1H- pyrazol-1- yl]pyrimidin- 5- yl}pyridine- 3-carboxylate |
| Example 670^{m)} | 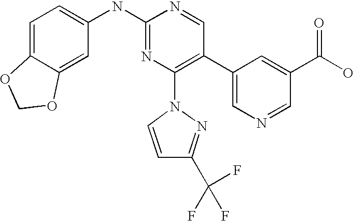 5-{2-(1,3-benzodioxol-5-ylamino)-4-[3-(trifluoromethyl)-1H-pyrazol-1- yl]pyrimidin-5-yl}pyridine-3-carboxylic acid | MS(ES): 471 (M + 1) for C₂₁H₁₃F₃N₆O₄ 400 MHz, DMSO-d₆: δ 6.00 (d, J = 9.60 Hz, 2H), 6.92 (d, J = 8.40 Hz, 1H), 7.03 (d, J = 2.60 Hz, 1H), 7.17 (d, J = 8.44 Hz, 1H), 7.44 (s, 1H), 7.99 (s, 1H), 8.53 (s, 1H), 8.63 (d, J = 1.92 Hz, 1H), 8.75 (s, 1H), 8.99 (s, 1H), 10.13 (s, 1H), 13.39 (br s, 1H). | Example 663 ethyl 5-{2- (1,3- benzodioxol- 5-ylamino)- 4-[3- (trifluoro- methyl)-1H- pyrazol-1- yl]pyrimidin- 5- yl}pyridine- 3-carboxylate |
| Example 671^{m)} | 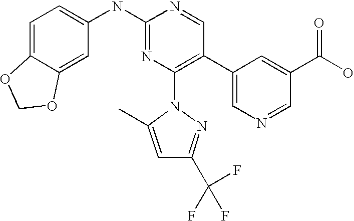 5-{2-(1,3-benzodioxol-5-ylamino)-4-[5-methyl-3-(trifluoromethyl)-1H- pyrazol-1-yl]pyrimidin-5-yl}pyridine-3-carboxylic acid | MS(ES): 485 (M + 1) for C₂₂H₁₅F₃N₆O₄ 400 MHz, DMSO-d₆: δ 2.39 (s, 3H), 6.00 (s, 2H), 6.74 (s, 1H), 6.91 (d, J = 8.44 Hz, 1H), 7.12 (t, J = 1.52 Hz, 1H), 7.39 (s, 1H), 7.86 (t, J = 79.00 Hz, 1H), 8.52 (d, J = 2.04 Hz, 1H), 8.91 (s, 1H), 8.94 (d, J = 1.72 Hz, 1H), 10.16 (s, 1H), 13.37 (br s, 1H). | Example 664 ethyl 5-{2- (1,3- benzodioxol- 5-ylamino)- 4-[5-methyl- 3- (trifluoro- methyl)-1H- pyrazol-1- yl]pyrimidin- 5- yl}pyridine- 3-carboxylate |
| Example 672ⁿ⁾ | 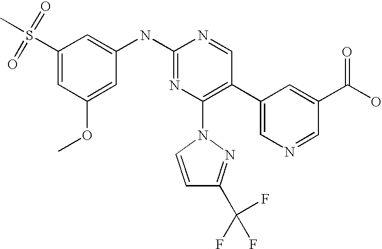 5-(2-{[3-methoxy-5-(methylsulfonyl)phenyl]amino}-4-[3-(trifluoromethyl)- 1H-pyrazol-1-yl]pyrimidin-5-yl)pyridine-3-carboxylic acid | MS(ES): 535 (M + 1) for C₂₂H₁₇F₃N₆O₅S. 400 MHz, DMSO-d₆: δ 3.24 (s, 3H), 3.87 (s, 3H), 7.05 (d, J = 2.64 Hz, 1H), 7.12 (t, J = 2.00 Hz, 1H), 7.70 (s, 1H), 8.03 (t, J = 1.88 Hz, 1H), 8.16 (s, 1H), 8.54-8.57 (m, 2H), 8.84 (s, 1H), 8.97 (d, J = 1.76 Hz, 1H), 10.63 (s, 1H). | Example 665 ethyl 5-(2- {[3-methoxy- 5- (methylsulfonyl) phenyl]amino}- 4-[3- (trifluoromethyl)- 1H-pyrazol-1- yl]pyrimidin- 5-yl)pyridine- 3-carboxylate |
| Example 673^{o)} | 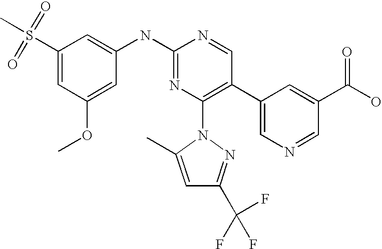 5-(2-{[3-methoxy-5-(methylsulfonyl)phenyl]amino}-4-[5-methyl-3- (trifluoromethyl)-1H-pyrazol-1-yl]pyrimidin-5-yl)pyridine-3-carboxylic acid | MS(ES): 549 (M + 1) for C₂₃H₁₉F₃N₆O₅S. 400 MHz, DMSO-d₆: δ 2.47 (s, 3H), 3.23 (s, 3H), 3.86 (s, 3H), 6.79 (s, 1H), 7.14 (s, 1H), 7.73 (s, 1H), 7.86 (s, 1H), 8.03 (s, 1H), 8.51 (s, 1H), 8.96 (s, 1H), 9.04 (s, 1H), 10.65 (s, 1H), 13.49 (s, 1H). | Example 666 ethyl 5-(2- {[3-methoxy- 5- (methyl- sulfonyl)phenyl] amino}-4-[5- methyl-3- (trifluoro- methyl)-1H- pyrazol-1- yl]pyrimidin- 5- yl)pyridine- 3-carboxylate |

^{l)}NaOH (4 eq), THF-H₂O, RT, 4 h
^{m)}NaOH (2 eq), THF-H₂O, 40° C. 6-14 h
ⁿ⁾Ba(OH)₂, dioxane-H₂O, RT, 12-24 h
^{o)}Ba(OH)₂, dioxane-H₂O, 55° C., 1 h.

### Example 674

### methyl 5-{2-[(3,5-dimethoxyphenyl)amino]-4-[3-(trifluoromethyl)-1H-pyrazol-1-yl]pyrimidin-5-yl}-2-methoxypyridine-3-carboxylate

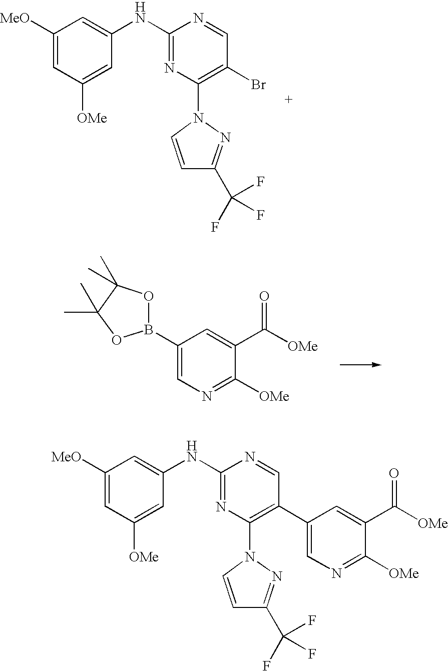

A suspension of 5-bromo-N-(3,5-dimethoxyphenyl)-4-[3-(trifluoromethyl)-1H-pyrazol-1-yl]pyrimidin-2-amine (Intermediate 215, 0.36 mmol, 160 mg), methyl 2-methoxy-5-(4,4,5,5-tetramethyl-1,3,2-dioxaborolan-2-yl)pyridine-3-carboxylate (0.36 mmol, 106 mg), [1,1′-bis(diphenylphosphino)ferrocene]dichloropalladium(II) complex with CH₂Cl₂ (0.073 mmol, 60 mg) and sodium carbonate (0.36 mmol, 40 mg) in acetonitrile/water (5:1) was degassed and heated to 100° C. for 45 min under an inert atmosphere. The reaction mass was passed through a celite bed. The solvent was concentrated in vacuo and the resultant crude mass was taken in EtOAc (50 mL), washed with water, brine, dried over Na₂SO₄, filtered and concentrated. The crude material was purified by silica gel column chromatography to give the title compound (120 mg).

| | | Mass spectrum and ¹H | |
|---|---|---|---|
| Compound | Structure | NMR | SM |
| Example 674 | 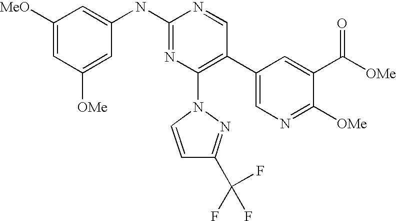 methyl 5-{2-[(3,5-dimethoxyphenyl)amino]-4-[3-(trifluoromethyl)- 1H-pyrazol-1-yl]pyrimidin-5-yl}-2-methoxypyridine-3-carboxylate | MS(ES): 531 (M + 1) for C₂₄H₂₁F₃N₆O₅. 400 MHz, DMSO-d₆: δ 3.73 (s, 6H), 3.76 (s, 3H), 3.93 (s, 3H), 6.20 (t, J = 2.20 Hz, 1H), 7.03 (d, J = 2.64 Hz, 1H), 7.09 (d, J = 2.20 Hz, 2H), 7.85 (d, J = 2.48 Hz, 1H), 8.27 (d, J = 2.48 Hz, 1H), 8.48 (t, J = 1.68 Hz, 1H), 8.77 (s, 1H), 10.18 (s, 1H). | Intermediate 215 5-bromo-N- (3,5- dimethoxy- phenyl)-4-[3- (trifluoro- methyl)-1H- pyrazol-1- yl]pyrimidin- 2-amine |

### Example 675

### 5-{2-[(3,5-dimethoxyphenyl)amino]-4-[3-(trifluoromethyl)-1H-pyrazol-1-yl]pyrimidin-5-yl}-2-methoxypyridine-3-carboxylic acid

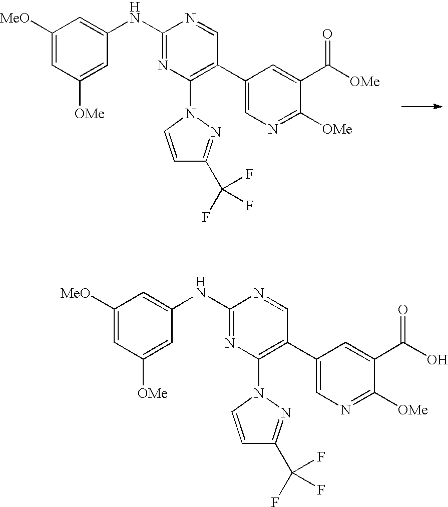

To 120 mg of methyl 5-{2-[(3,5-dimethoxyphenyl)amino]-4-[3-(trifluoromethyl)-1H-pyrazol-1-yl]pyrimidin-5-yl}-2-methoxypyridine-3-carboxylate (Example 674, 0.22 mmol) dissolved in a mixture of dioxane (1 mL) and water (0.33 mL), was added Barium hydroxide monohydrate (0.6 mmol, 114 mg) and allowed to stir overnight at room temperature. After completion of the reaction, the mixture was then carefully acidified with 1 N HCl and then diluted with ethyl acetate (50 mL), washed with water, brine, dried over Na₂SO₄ and concentrated to yield 70 mg of the title compound.

| | | Mass spectrum and ¹H | |
|---|---|---|---|
| Compound | Structure | NMR | SM |
| Example 675 | 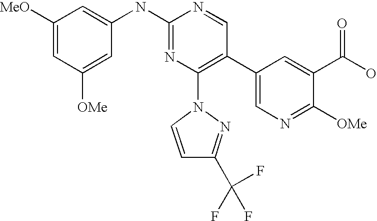 5-{2-[(3,5-dimethoxyphenyl)amino]-4-[3-(trifluoromethyl)- 1H-pyrazol-1-yl]pyrimidin-5-yl}-2-methoxypyridine-3- carboxylic acid | MS(ES): 517 (M + 1) for C₂₃H₁₉F₃N₆O₅ 400 MHz, DMSO-d₆: δ 3.75 (s, 6H), 3.91 (s, 3H), 6.21 (t, J = 2.16 Hz, 1H), 7.02 (d, J = 2.64 Hz, 1H), 7.11 (d, J = 2.16 Hz, 2H), 7.77 (s, 1H), 8.16 (s, 1H), 8.43 (s, 1H), 8.76 (s, 1H), 10.17 (s, 1H). | Example 674 methyl 5-{2- [(3,5- dimethoxyphenyl) amino]-4-[3- (trifluoromethyl)- 1H-pyrazol-1- yl]pyrimidin-5- yl}-2- methoxypyridine- 3-carboxylate |

### Example 676

### methyl 5-{2-[(3,5-dimethoxyphenyl)amino]-4-[5-methyl-3-(trifluoromethyl)-1H-pyrazol-1-yl]pyrimidin-5-yl}-2-methoxypyridine-3-carboxylate

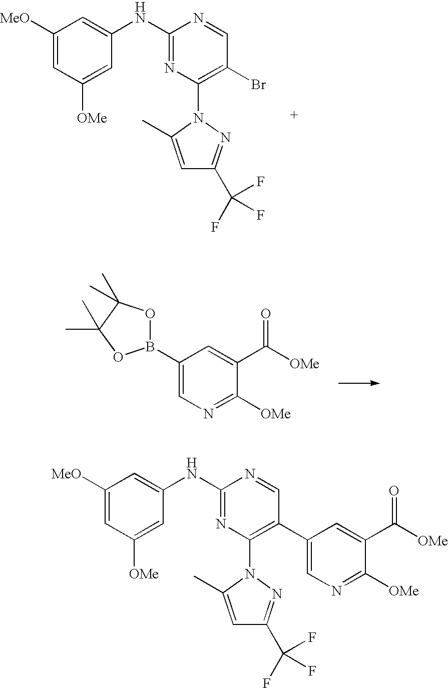

A suspension of 5-bromo-N-(3,5-dimethoxyphenyl)-4-[5-methyl-3-(trifluoromethyl)-1H-pyrazol-1-yl]pyrimidin-2-amine (Intermediate 216, 0.44 mmol, 200 mg) methyl 2-methoxy-5-(4,4,5,5-tetramethyl-1,3,2-dioxaborolan-2-yl)pyridine-3-carboxylate (0.48 mmol, 141 mg), [1,1′-bis(diphenylphosphino)ferrocene]dichloropalladium(II) (0.087 mmol, 64 mg) and sodium carbonate (0.44 mmol, 46 mg) in acetonitrile/water (8:2) was degassed and heated to 90° C. for 15 min under an inert atmosphere. After completion of the reaction, the reaction mass was diluted with ethyl acetate (30 mL). The organic layer was separated, washed with water and brine, dried over Na₂SO₄, filtered and concentrated. The crude material was purified by silica gel column chromatography using ethyl acetate/hexanes (45:55) to obtain 150 mg of Example 676.

| | | Mass spectrum and ¹H | |
|---|---|---|---|
| Compound | Structure | NMR | SM |
| Example 676 | 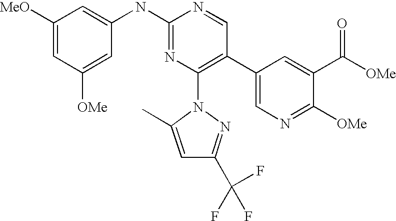 methyl 5-{2-[(3,5-dimethoxyphenyl)amino]-4-[5-methyl-3- (trifluoromethyl)-1H-pyrazol-1-yl]pyrimidin-5-yl}-2- methoxypyridine-3-carboxylate | MS(ES): 545 (M + 1) for C₂₅H₂₃F₃N₆O₅. 400 MHz, DMSO-d₆: δ 2.29 (s, 3H), 3.71 (s, 6H), 3.74 (s, 3H), 3.91 (s, 3H), 6.21 (t, J = 2.16 Hz, 1H), 6.75 (s, 1H), 7.04 (d, J = 2.12 Hz, 2H), 7.60 (d, J = 2.52 Hz, 1H), 8.22 (d, J = 2.52 Hz, 1H), 8.93 (s, 1H), 10.21 (s, 1H). | Intermediate 216 5-bromo-N- (3,5- dimethoxy- phenyl)-4-[5- methyl-3- (trifluoro- methyl)-1H- pyrazol-1- yl]pyrimidin- 2-amine |

### Example 677

### 5-{2-[(3,5-dimethoxyphenyl)amino]-4-[5-methyl-3-(trifluoromethyl)-1H-pyrazol-1-yl]pyrimidin-5-yl}-2-methoxypyridine-3-carboxylic acid

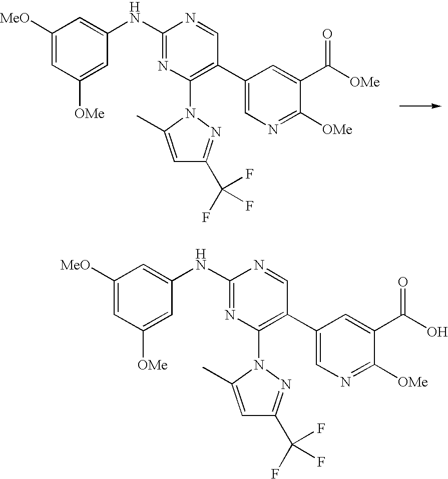

To 150 mg of methyl 5-[2-[(3,5-dimethoxyphenyl)amino]-4-[5-methyl-3-(trifluoromethyl)-1H-pyrazol-1-yl]pyrimidin-5-yl}-2-methoxypyridine-3-carboxylate (Example 676, 0.27 mmol) taken in a mixture of tetrahydrofuran (1.5 mL), was added 1 N aq. sodium hydroxide (1.07 mmol) and stirred in a room temperature for 4 h. After completion of reaction, reaction mixture was then carefully acidified with 1 N HCl and the solid that precipitated was filtered, washed with water and dried in vacuo to yield 120 mg Example 677.

| | | Mass spectrum and ¹H | |
|---|---|---|---|
| Compound | Structure | NMR | SM |
| Example 677 | 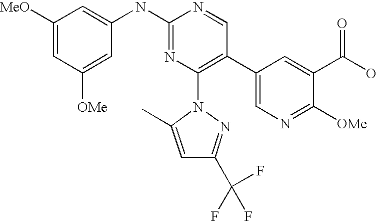 5-{2-[(3,5-dimethoxyphenyl)amino]-4-[5-methyl-3- (trifluoromethyl)-1H-pyrazol-1-yl]pyrimidin-5-yl}-2- methoxypyridine-3-carboxylic acid | MS(ES): 531 (M + 1) for C₂₄H₂₁F₃N₆O₅. 400 MHz, DMSO-d₆: δ 2.30 (s, 3H), 3.71 (s, 6H), 3.89 (s, 3H), 6.20 (s, 1H), 6.74 (s, 1H), 7.04 (s, 2H), 7.61 (t, J = 1.52 Hz, 1H), 8.14 (d, J = 1.76 Hz, 1H), 8.92 (s, 1H), 10.20 (s, 1H), 13.10 (s, 1H). | Example 676 methyl 5- {2-[(3,5- dimethoxy- phenyl)amino]- 4-[5- methyl-3- (trifluoro- methyl)-1H- pyrazol-1- yl]pyrimidin- 5-yl}-2- methoxy- pyridine-3- carboxylate |

### Example 678

### 5-{2-[(3,5-dimethoxyphenyl)amino]-4-[3-(trifluoromethyl)-1H-pyrazol-1-yl]pyrimidin-5-yl}-N-methylpyridine-3-carboxamide

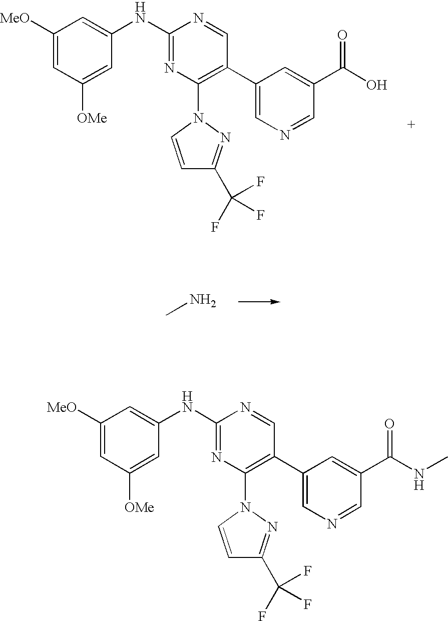

To a mixture of 5-{2-[(3,5-dimethoxyphenyl)amino]-4-[3-(trifluoromethyl)-1H-pyrazol-1-yl]pyrimidin-5-yl}pyridine-3-carboxylic acid (Example 635, 0.56 mmol, 270 mg), triethylamine (1.67 mmol, 0.23 mL, 0.17 g) and methylamine hydrochloride (1.11 mmol, 75 mg) in dichloromethane was added T₃P in 50% EtOAc (1.11 mmol, 0.7 mL, 353 mg) at 0° C. The reaction mixture was slowly raised to room temperature and stirred overnight. After completion of the reaction, the mixture was then diluted with dichloromethane (15 mL), and the organic layer was successively washed with water (2×20 mL), 10% aq sodium bicarbonate solution (15 mL) and brine. The organic layer was dried over sodium sulphate and concentrated. The crude material was purified by silica gel column chromatography (230-400 mesh) using 8% methanol/chloroform to yield 80 mg of the title compound.

| | | Mass spectrum and ¹H | |
|---|---|---|---|
| Compound | Structure | NMR | SM |
| Example 678 | 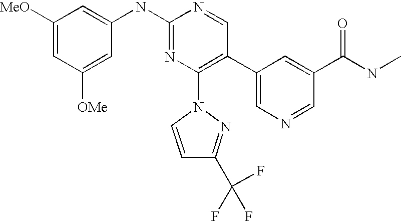 5-{2-[(3,5-dimethoxyphenyl)amino]-4-[3-(trifluoromethyl)-1H- pyrazol-1-yl]pyrimidin-5-yl}-N-methylpyridine-3-carboxamide | MS(ES): 500 (M + 1) for C₂₃H₂₀F₃N₇O₃. 400 MHz, DMSO-d₆: δ 2.79 (d, J = 4.44 Hz, 3H), 3.74 (s, 6H), 6.21 (t, J = 1.96 Hz, 1H), 7.04 (d, J = 2.52 Hz, 1H), 7.09 (d, J = 2.04 Hz, 2H), 8.06 (t, J = 1.88 Hz, 1H), 8.42 (d, J = 2.08 Hz, 1H), 8.50 (d, J = 1.12 Hz, 1H), 8.64 (m, J = 4.52 Hz, 1H), 8.80 (s, 1H), 8.91 (d, J = 1.84 Hz, 1H), 10.24 (s, 1H). | Example 635 5-{2-[(3,5- dimethoxy- phenyl)amino]- 4-[3- (trifluoro- methyl)-1H- pyrazol-1- yl]pyrimidin- 5- yl}pyridine- 3-carboxylic acid |

### Example 679

### 5-{2-[(3,5-dimethoxyphenyl)amino]-4-[5-methyl-3-(trifluoromethyl)-1H-pyrazol-1-yl]pyrimidin-5-yl}-N-methylpyridine-3-carboxamide

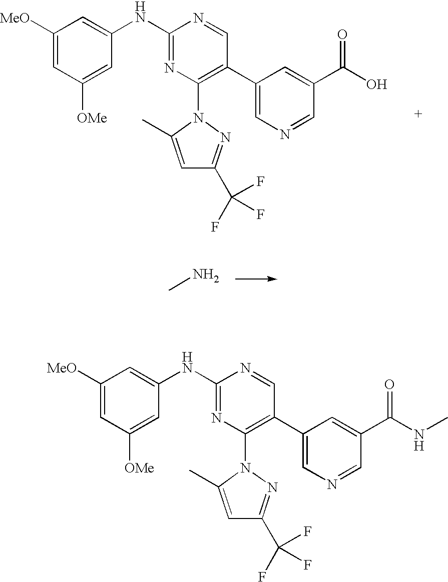

To a mixture of 5-{2-[(3,5-dimethoxyphenyl)amino]-4-[5-methyl-3-(trifluoromethyl)-1H-pyrazol-1-yl]pyrimidin-5-yl}pyridine-3-carboxylic acid (Example 636, 0.35 mmol, 175 mg), triethylamine (1.05 mmol, 0.14 mL, 106 mg) and methylamine hydrochloride (0.70 mmol, 47 mg) in dichloromethane (10 mL) was added T₃P (50% EtOAc) (0.70 mmol, 0.45 mL, 223 mg) slowly at 0° C. The reaction mixture was slowly raised to room temperature and stirred for 3 h. The reaction mixture was then diluted with dichloromethane (15 mL) and the dichloromethane layer was washed successively with water (2×20 mL), 10% aq sodium bicarbonate solution (15 mL) and brine. The organic layer was dried over sodium sulphate, filtered, concentrated and dried to yield 120 mg of the title compound.

| | | Mass spectrum and ¹H | |
|---|---|---|---|
| Compound | Structure | NMR | SM |
| Example 679 | 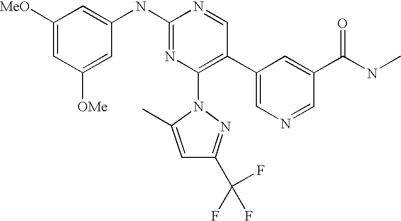 5-{2-[(3,5-dimethoxyphenyl)amino]-4-[5-methyl-3- (trifluoromethyl)-1H-pyrazol-1-yl]pyrimidin-5-yl}-N- methylpyridine-3-carboxamide | MS(ES): 514 (M + 1) for C₂₄H₂₂F₃N₇O₃. 400 MHz, DMSO-d₆: δ 2.38 (s, 3H), 2.79 (d, J = 4.44 Hz, 3H), 3.73 (s, 6H), 6.23 (s, 1H), 6.76 (s, 1H), 7.05 (d, J = 1.72 Hz, 2H), 7.98 (s, 1H), 8.22 (d, J = 1.92 Hz, 1H), 8.63 (m, J = 4.56 Hz, 1H), 8.86 (d, J = 1.72 Hz, 1H), 8.96 (s, 1H), 10.26 (s, 1H). | Example 636 5-{2-[(3,5- dimethoxy- phenyl)amino]- 4-[5-methyl- 3- (trifluoro- methyl)-1H- pyrazol-1- yl]pyrimidin- 5- yl}pyridine- 3-carboxylic acid |

### Example 680

### 5-{2-[(3,5-dimethoxyphenyl)amino]-4-[3-(trifluoromethyl)-1H-pyrazol-1-yl]pyrimidin-5-yl}-N-methoxypyridine-3-carboxamide

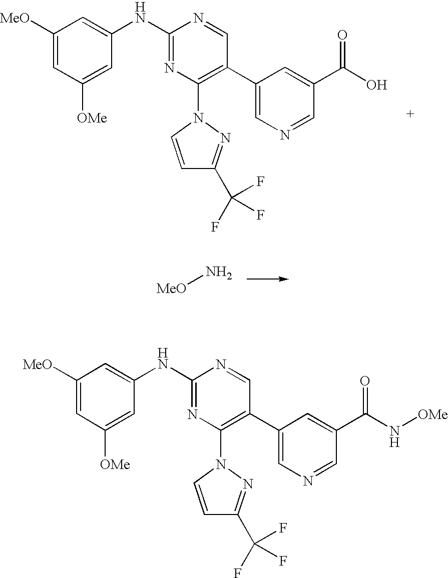

To a mixture of 5-{2-[(3,5-dimethoxyphenyl)amino]-4-[3-(trifluoromethyl)-1H-pyrazol-1-yl]pyrimidin-5-yl}pyridine-3-carboxylic acid (Example 635, 0.41 mmol, 200 mg), triethylamine (0.82 mmol, 0.12 mL, 80 mg) and methoxylamine hydrochloride (0.5 mmol, 42 mg) in dichloromethane, was added. TBTU (0.49 mmol, 158 mg) slowly at 0° C. The reaction mixture was slowly raised to room temperature and stirred overnight. The reaction mixture was then diluted with dichloromethane (15 mL) and the dichloromethane solution was washed successively with water (2×20 mL), 10% aq sodium bicarbonate solution (20 mL) and brine. The organic layer was dried over sodium sulphate, and concentrated. The crude material was purified by silica gel column chromatography using 8% methanol/chloroform to yield 90 mg of the title compound.

| | | Mass spectrum and ¹H | |
|---|---|---|---|
| Compound | Structure | NMR | SM |
| Example 680 | 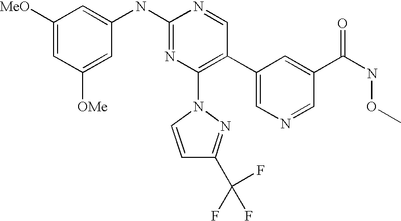 5-{2-[(3,5-dimethoxyphenyl)amino]-4-[3-(trifluoromethyl)-1H- pyrazol-1-yl]pyrimidin-5-yl}-N-methoxypyridine-3-carboxamide | MS(ES): 516 (M + 1) for C₂₃H₂₀F₃N₇O₄. 400 MHz, DMSO-d₆: δ 3.73 (s, 3H), 3.75 (s, 6H), 6.23 (t, J = 2.16 Hz, 1H), 7.06 (d, J = 2.60 Hz, 1H), 7.11 (d, J = 2.16 Hz, 2H), 8.00 (s, 1H), 8.50 (d, J = 2.12 Hz, 1H), 8.53 (d, J = 1.60 Hz, 1H), 8.81 (s, 1H), 8.84 (d, J = 1.96 Hz, 1H), 10.26 (s, 1H), 11.97 (s, 1H). | Example 635 5-{2-[(3,5- dimethoxy- phenyl)amino]- 4-[3- (trifluoro- methyl)-1H- pyrazol-1- yl]pyrimidin- 5- yl}pyridine- 3-carboxylic acid |

### Example 681

### 5-{2-[(3,5-dimethoxyphenyl)amino]-4-[5-methyl-3-(trifluoromethyl)-1H-pyrazol-1-yl]pyrimidin-5-yl}-N-methoxypyridine-3-carboxamide

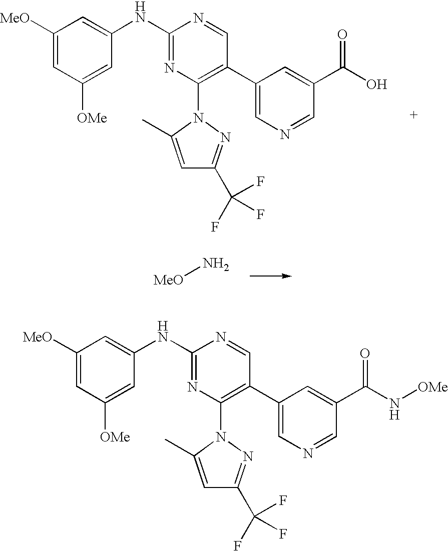

To a mixture of 5-{2-[(3,5-dimethoxyphenyl)amino]-4-[5-methyl-3-(trifluoromethyl)-1H-pyrazol-1-yl]pyrimidin-5-yl}pyridine-3-carboxylic acid (Example 636, 0.34 mmol, 170 mg), triethylamine (0.68 mmol, 0.1 mL) and methoxylamine hydrochloride (0.51 mmol, 43 mg) in dichloromethane, was added TBTU (131 mg, 0.41 mmol, 1.2 eq) at 0° C. The reaction mixture was slowly raised to room temperature and stirred overnight. The reaction mixture was then diluted with dichloromethane (15 mL) and the dichloromethane solution was washed successively with water (2×20 mL), 10% aq. sodium bicarbonate solution (20 mL) and brine. The organic layer was dried over sodium sulphate, filtered, concentrated and dried to yield 120 mg of the title compound.

| | | Mass spectrum and ¹H | |
|---|---|---|---|
| Compound | Structure | NMR | SM |
| Example 681 | 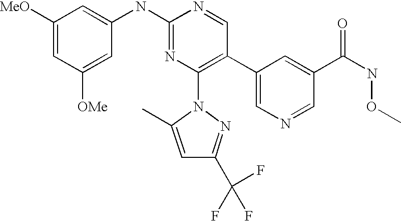 5-{2-[(3,5-dimethoxyphenyl)amino]-4-[5-methyl-3- (trifluoromethyl)-1H-pyrazol-1-yl]pyrimidin-5-yl}-N- methoxypyridine-3-carboxamide | MS(ES): 530 (M + 1) for C₂₄H₂₂F₃N₇O₄. 400 MHz, DMSO-d₆: δ 2.41 (s, 3H), 3.72 (s, 3H), 3.74 (s, 6H), 6.24 (t, J = 2.08 Hz, 1H), 6.78 (s, 1H), 7.06 (d, J = 1.96 Hz, 2H), 7.86 (s, 1H), 8.32 (d, J = 1.80 Hz, 1H), 8.79 (d, J = 1.84 Hz, 1H), 8.97 (s, 1H), 10.28 (s, 1H), 11.96 (s, 1H). | Example 636 5-{2-[(3,5- dimethoxy- phenyl)amino]- 4-[5-methyl- 3- (trifluoro- methyl)-1H- pyrazol-1- yl]pyrimidin- 5- yl}pyridine- 3-carboxylic acid |

### Example 682

### 5-{2-[(3,5-dimethoxyphenyl)amino]-4-[3-(trifluoromethyl)-1H-pyrazol-1-yl]pyrimidin-5-yl}-N-(2-hydroxyethyl)pyridine-3-carboxamide

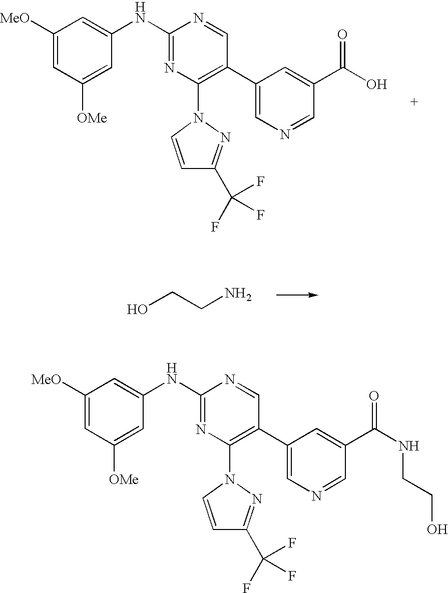

To a mixture of T₃P (4.11 mmol, 1.3 g) and Et₃N (5.14 mmol, 520 mg) taken in dry dichloromethane (20 mL), was added ethanolamine (2.03 mmol, 128 mg). Then 5-{2-[(3,5-dimethoxyphenyl)amino]-4-[3-(trifluoromethyl)-1H-pyrazol-1-yl]pyrimidin-5-yl}pyridine-3-carboxylic acid (Example 635, 1.03 mmol, 500 mg) in dry THF (20 mL) was added slowly for 15 min. The reaction mixture was stirred overnight at room temperature. The solvent was removed in vacuo, water was added and extracted with EtOAc. The organic layer was further washed with brine, dried over Na₂SO₄ and concentrated. The crude mass was purified by column chromatography (230-400 mesh) using 10% MeOH/dichloromethane as an eluent to yield 120 mg product. The compound was further purified by RP-HPLC (Atlantis C18 column (19×250 mm; 10 μm); using a binary solvent mixture of 0.1% TFA in water (A)/MeOH (B) (0-20 min: 10-65% B, 20-30 min: 65-75% B and 30-40 min: 75-100% B; flow rate of 15 mL/min; Separation was monitored at 210 and 290 nm) to get 0.057 g of the pure title compound

| | | Mass spectrum and ¹H | |
|---|---|---|---|
| Compound | Structure | NMR | SM |
| Example 682 | 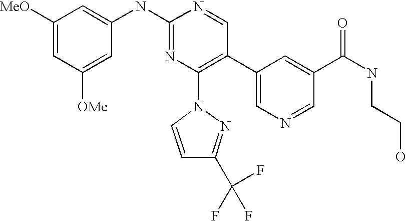 5-{2-[(3,5-dimethoxyphenyl)amino]-4-[3-(trifluoromethyl)-1H- pyrazol-1-yl]pyrimidin-5-yl}-N-(2-hydroxyethyl)pyridine-3- carboxamide | MS(ES): 530 (M + 1) for C₂₄H₂₂F₃N₇O₄. 400 MHz, DMSO-d₆: δ 3.34 (q, J = 6.00 Hz, 2H), 3.51 (t, J = 6.04 Hz, 2H), 3.74 (s, 6H), 6.21 (t, J = 1.92 Hz, 1H), 7.04 (d, J = 2.52 Hz, 1H), 7.10 (d, J = 2.04 Hz, 2H), 8.14 (t, J = 1.72 Hz, 1H), 8.41 (d, J = 1.92 Hz, 1H), 8.50 (s, 1H), 8.67 (t, J = 5.48 Hz, 1H), 8.81 (s, 1H), 8.93 (d, J = 1.80 Hz, 1H), 10.25 (s, 1H). | Example 635 5-{2-[(3,5- dimethoxy- phenyl)amino]- 4-[3- (trifluoro- methyl)-1H- pyrazol-1- yl]pyrimidin- 5- yl}pyridine- 3-carboxylic acid |

### Example 683

### 5-{2-[(3,5-dimethoxyphenyl)amino]-4-[5-methyl-3-(trifluoromethyl)-1H-pyrazol-1-yl]pyrimidin-5-yl}-N-(2-hydroxyethyl)pyridine-3-carboxamide

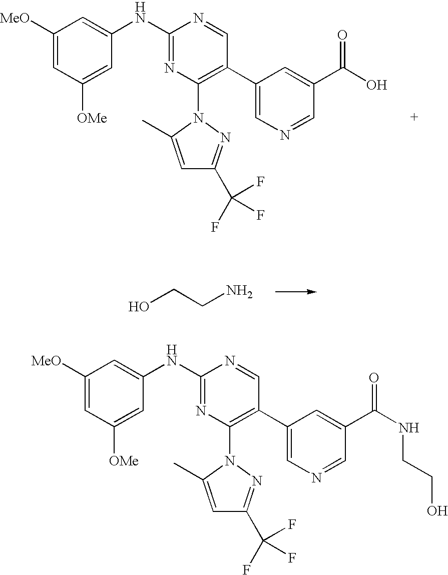

To 100 mg of 5-{2-[(3,5-dimethoxyphenyl)amino]-4-[5-methyl-3-(trifluoromethyl)-1H-pyrazol-1-yl]pyrimidin-5-yl}pyridine-3-carboxylic acid (Example 636, 0.19 mmol) in dry dichloromethane (1 mL), was added Et₃N (1.19 mmol, 0.121 g) with constant stiffing. To this solution, TBTU (0.51 mmol, 0.167 g) and HOBt (0.51 mmol, 0.070 g) were added and the reaction mixture was stirred for 15 min. Then ethanolamine (0.47 mmol, 0.029 g) was added and allowed to stir for 12 h. The reaction mixture was diluted with dichloromethane and the organic layer was further washed with brine and 10% NaHCO₃ solution, dried over Na₂SO₄ and concentrated. The crude mass was purified by column chromatography (230-400 mesh) using 2% MeOH/CHCl₃ as an eluent to yield a solid. The solid obtained was dissolved in minimum amount of dry dichloromethane and n-hexane was added to it and stirred for 1 h. The precipitate was filtered and dried under vacuum to give the title compound as a brown solid (20 mg).

| | | Mass spectrum and ¹H | |
|---|---|---|---|
| Compound | Structure | NMR | SM |
| Example 683 | 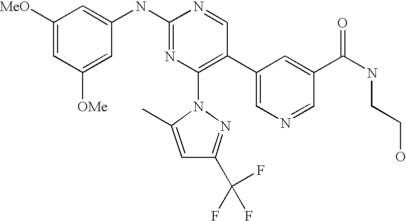 5-{2-[(3,5-dimethoxyphenyl)amino]-4-[5-methyl-3- (trifluoromethyl)-1H-pyrazol-1-yl]pyrimidin-5-yl}-N-(2- hydroxyethyl)pyridine-3-carboxamide | MS(ES): 544 (M + 1) and 545 (M + 2) for C₂₅H₂₄F₃N₇O₄. 400 MHz, DMSO-d₆: δ 2.38 (s, 3H), 3.34 (m, 2H, merges with water peak), 3.51 (q, J = 5.84 Hz, 2H), 3.72 (s, 6H), 4.76 (t, J = 5.60 Hz, 1H), 6.22 (d, J = 2.00 Hz, 1H), 6.76 (s, 1H), 7.05 (d, J = 1.80 Hz, 2H), 8.04 (d, J = 1.84 Hz, 1H), 8.18 (d, J = 1.96 Hz, 1H), 8.66 (t, J = 5.48 Hz, 1H), 8.88 (d, J = 1.80 Hz, 1H), 8.97 (s, 1H), 10.26 (s, 1H). | Example 636 5-{2-[(3,5- dimethoxy- phenyl)amino]- 4-[5-methyl- 3- (trifluoro- methyl)-1H- pyrazol-1- yl]pyrimidin- 5- yl}pyridine- 3-carboxylic acid |

### Example 684

### 5-{2-[(3,5-dimethoxyphenyl)amino]-4-[3-(trifluoromethyl)-1H-pyrazol-1-yl]pyrimidin-5-yl}-N-[2-(methylsulfony)ethyl]pyridine-3-carboxamide

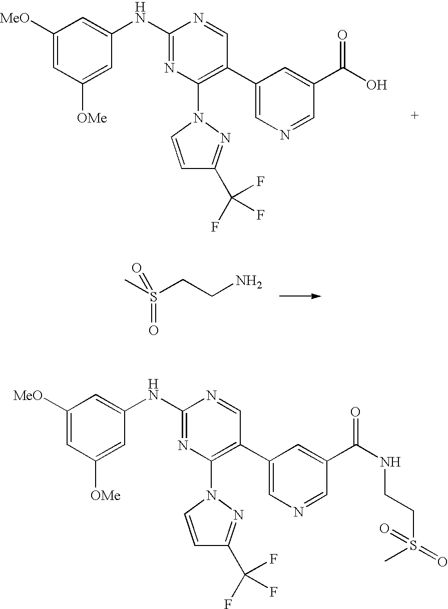

To 100 mg of 5-{2-[(3,5-dimethoxyphenyl)amino]-4-[3-(trifluoromethyl)-1H-pyrazol-1-yl]pyrimidin-5-yl}pyridine-3-carboxylic acid (Example 635, 0.21 mmol) taken in dry dichloromethane (1 mL), was added Et₃N (1.23 mmol, 0.125 g) with constant stiffing. To this solution, TBTU (0.53 mmol, 0.171 g) followed by HOBt (0.53 mmol, 0.072 g) were added and the reaction mixture was left for stirring for 15 min. Then 2-aminoethylmethylsulfone hydrochloride (0.38 mmol, 61 mg) was added and allowed to stir for 12 h. The reaction mixture was diluted with dichloromethane and the organic layer was further washed with brine and 10% NaHCO₃ solution, dried over Na₂SO₄ and concentrated. The crude mass was purified by column chromatography (230-400 mesh) using 1.5% MeOH/CHCl₃ as an eluent to yield a solid. The solid obtained was dissolved in minimum amount of dry dichloromethane and n-hexane was added to it and stirred for 1 h. The precipitate was filtered and dried under vacuum to yield the title compound as a fine white solid (30 mg).

| | | Mass spectrum and ¹H | |
|---|---|---|---|
| Compound | Structure | NMR | SM |
| Example 684 | 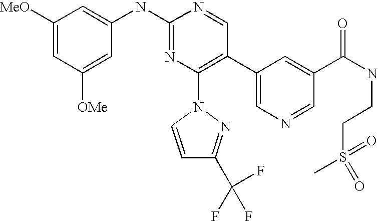 5-{2-[(3,5-dimethoxyphenyl)amino]-4-[3-(trifluoromethyl)- 1H-pyrazol-1-yl]pyrimidin-5-yl}-N-[2-(methylsulfonyl)ethyl] pyridine-3-carboxamide | MS(ES): 592 (M + 1) for C₂₅H₂₄F₃N₇O₅S. 400 MHz, DMSO-d₆: δ 3.05 (s, 3H), 3.39 (t, J = 6.72 Hz, 2H), 3.70 (q, J = 6.12 Hz, 2H), 3.75 (s, 6H), 6.23 (t, J = 2.04 Hz, 1H), 7.06 (d, J = 2.52 Hz, 1H), 7.11 (d, J = 2.08 Hz, 2H), 8.10 (t, J = 2.00 Hz, 1H), 8.46 (d, J = 2.00 Hz, 1H), 8.52 (s, 1H), 8.81 (s, 1H), 8.93 (d, J = 1.88 Hz, 1H), 8.97 (t, 1H), 10.26 (s, 1H). | Example 635 5-{2-[(3,5- dimethoxy- phenyl)amino]- 4-[3- (trifluoro- methyl)-1H- pyrazol-1- yl]pyrimidin- 5- yl}pyridine- 3-carboxylic acid |

### Example 685

### 5-{2-[(3,5-dimethoxyphenyl)amino]-4-[5-methyl-3-(trifluoromethyl)-1H-pyrazol-1-yl]pyrimidin-5-yl}-N-[2-(methylsulfonyl)ethyl]pyridine-3-carboxamide

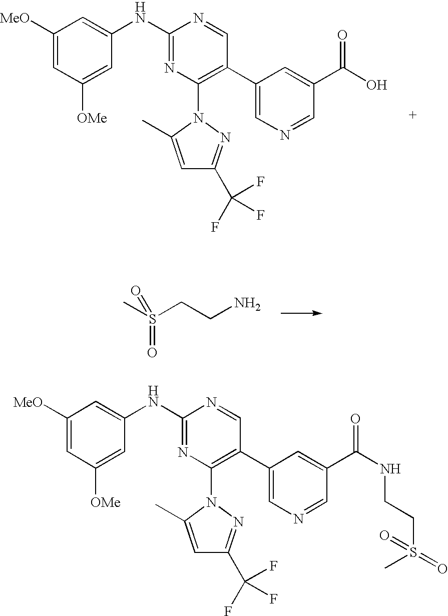

To 100 mg of 5-{2-[(3,5-dimethoxyphenyl)amino]-4-[5-methyl-3-(trifluoromethyl)-1H-pyrazol-1-yl]pyrimidin-5-yl}pyridine-3-carboxylic acid (Example 636, 0.19 mmol) taken in dry dichloromethane (1 mL), was added Et₃N (0.49 mmol, 0.051 g) with constant stirring. To this HATU (0.25 mmol, 0.099 g) followed by HOAt (0.25 mmol, 0.035 g) were added and stirred for 15 min. Then 2-aminoethylmethylsulfone hydrochloride (0.19 mmol, 0.03 g) was added and the reaction mixture allowed to stir for 12 h. The reaction mixture was diluted with dichloromethane and the organic layer was further washed with brine and 10% NaHCO₃ solution, dried over Na₂SO₄ and concentrated. The crude mass was purified by column chromatography (230-400 mesh) using 3% MeOH/CHCl₃ as an eluent to yield a solid. The solid obtained was dissolved in minimum amount of dry dichloromethane and n-hexane was added to it and stirred for 1 h. The precipitate was filtered and dried under vacuum to get the title compound as an off-white solid (60 mg).

| | | Mass spectrum and ¹H | |
|---|---|---|---|
| Compound | Structure | NMR | SM |
| Example 685 | 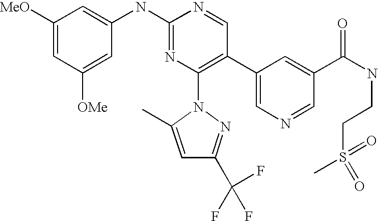 5-{2-[(3,5-dimethoxyphenyl)amino]-4-[5-methyl-3- (trifluoromethyl)-1H-pyrazol-1-yl]pyrimidin-5-yl}-N-[2- (methylsulfonyl)ethyl]pyridine-3-carboxamide | MS(ES): 606 (M + 1) for C₂₆H₂₆F₃N₇O₅S. 400 MHz, DMSO-d₆: δ 2.41 (s, 3H), 3.05 (s, 3H), 3.38 (t, J = 6.88 Hz, 2H), 3.69 (q, J = 6.60 Hz, 2H), 3.73 (s, 6H), 6.24 (t, J = 2.20 Hz, 1H), 6.78 (s, 1H), 7.06 (d, J = 2.08 Hz, 2H), 8.00 (t, J = 2.04 Hz, 1H), 8.25 (d, J = 2.12 Hz, 1H), 8.88 (d, J = 1.92 Hz, 1H), 8.96 (t, J = 5.44 Hz, 2H), 10.26 (s, 1H). | Example 636 5-{2-[(3,5- dimethoxy- phenyl)amino]- 4-[5-methyl- 3- (trifluoro- methyl)-1H- pyrazol-1- yl]pyrimidin- 5- yl}pyridine- 3-carboxylic acid |

### Example 686

### 5-{2-[(3,5-dimethoxyphenyl)amino]-4-[3-(trifluoromethyl)-1H-pyrazol-1-yl]pyrimidin-5-yl}-N-(methylsulfonyl)pyridine-3-carboxamide

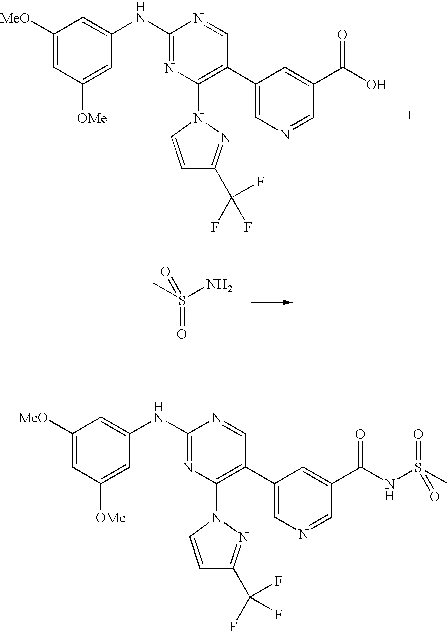

A suspension of 5-{2-[(3,5-dimethoxyphenyl)amino]-4-[3-(trifluoromethyl)-1H-pyrazol-1-yl]pyrimidin-5-yl}pyridine-3-carboxylic acid (Example 635, 0.41 mmol, 0.2 g), methane sulfonamide (1.02 mmol, 0.097 g), HATU (0.533 mmol, 0.2 g) HOAt (0.533 mmol, 0.072 g) and triethylamine (1.23 mmol, 0.124 g) in dichloromethane (2 mL) was stirred at room temperature for 4 h. The reaction mixture was diluted with dichloromethane (5 mL), washed with 10% sodium bicarbonate solution (5×2 mL), water (5 mL) and brine (5 mL), dried over anhydrous Na₂SO₄ and concentrated under reduced pressure. Then the crude mass was purified by RP-HPLC (Sunfire C18 column (19×250 mm; 10 μm); using a binary solvent mixture of 10 mM aq. NH₄OAc (A)/MeCN (B) (0-20 min: 10-60% B, 20-30 min: 60% B; 30-40 min: 60-70% B and 40-50 min: 70-100% B; flow rate of 15 mL/min; Separation was monitored at 210 and 300 nm) to get 0.04 g of Example 686.

| | | Mass spectrum and ¹H | |
|---|---|---|---|
| Compound | Structure | NMR | SM |
| Example 686 | 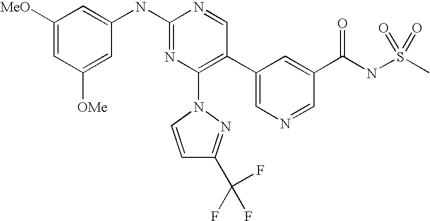 5-{2-[(3,5- dimethoxyphenyl)- amino]-4-[3- (trifluoromethyl)-1H- pyrazol-1- yl]pyrimidin-5-yl}-N- (methylsulfonyl)- pyridine-3-carboxamide | MS(ES): 564 (M + 1) for C₂₃H₂₀F₃N₇O₅S. 400 MHz, DMSO-d₆: δ 3.16 (s, 3H), 3.75 (s, 6H), 6.22 (d, J = 2.00 Hz, 1H), 7.04 (d, J = 2.44 Hz, 1H), 7.11 (d, J = 2.12 Hz, 2H), 8.18 (s, 1H), 8.42 (s, 1H), 8.49 (s, 1H), 8.80 (s, 1H), 8.98 (d, J = 1.80 Hz, 1H), 10.24 (s, 1H), 12.43 (s, 1H). | Example 635 5-{2-[(3,5- dimethoxy- phenyl)amino]- 4-[3- (trifluoro- methyl)-1H- pyrazol-1- yl]pyrimidin- 5- yl}pyridine- 3-carboxylic acid |

### Example 687

### 5-{2-[(3,5-dimethoxyphenyl)amino]-4-[5-methyl-3-(trifluoromethyl)-1H-pyrazol-1-yl]pyrimidin-5-yl}-N-(methylsulfonyl)pyridine-3-carboxamide

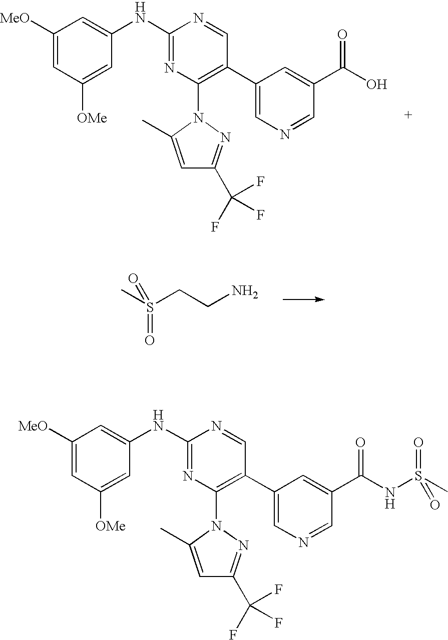

A suspension of 5-{2-[(3,5-dimethoxyphenyl)amino]-4-[5-methyl-3-(trifluoromethyl)-1H-pyrazol-1-yl]pyrimidin-5-yl}pyridine-3-carboxylic acid (Example 636, 0.1 g, 0.2 mmol), methane sulfonamide (0.5 mmol, 0.047 g), HATU (0.26 mmol, 0.099 g), HOAt (0.26 mmol, 0.035 g) and triethylamine (0.6 mmol, 0.061 g) in dichloromethane (1 mL) was stirred at room temperature for 4 h. After completion of reaction, the reaction mixture was diluted with dichloromethane (2 mL) and washed with 10% sodium bicarbonate solution (2×2 mL), water (2 mL) and brine (2 mL), dried over anhydrous Na₂SO₄ and concentrated under reduced pressure. Then the crude mass was purified by column chromatography (230-400 mesh) using 3% MeOH/CHCl₃ as an eluent to yield to get 0.04 g of Example 687.

| | | Mass spectrum and ¹H | |
|---|---|---|---|
| Compound | Structure | NMR | SM |
| Example 687 | 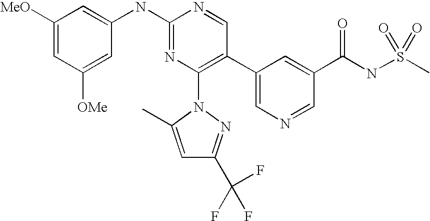 5-{2-[(3,5- dimethoxyphenyl)- amino]-4-[5-methyl-3- (trifluoromethyl)-1H- pyrazol-1- yl]pyrimidin-5-yl}-N- (methylsulfonyl)- pyridine-3-carboxamide | MS(ES): 578 (M + 1) for C₂₄H₂₂F₃N₇O₅S 400 MHz, DMSO-d₆: δ 2.32 (s, 3H), 2.95 (s, 3H), 3.72 (s, 6H), 6.21 (t, J = 2.16 Hz, 1H), 6.73 (s, 1H), 7.05 (d, J = 2.08 Hz, 2H), 8.06 (d, J = 1.96 Hz, 1H), 8.10 (d, J = 2.04 Hz, 1H), 8.92 (m, 2H), 10.23 (s, 1H). | Example 636 5-{2-[(3,5- dimethoxy- phenyl)amino]- 5-[5-methyl- 3-(trifluoro- methyl)-1H- pyrazol-1- yl]pyrimidin- 5-yl}pyridine- 3-carboxylic acid |

### General method for the synthesis of (2E)-3-(3-{2-[arylamino]-4-[1H-azol-1-yl]pyrimidin-5-yl}phenyl)prop-2-enoic acid derivatives

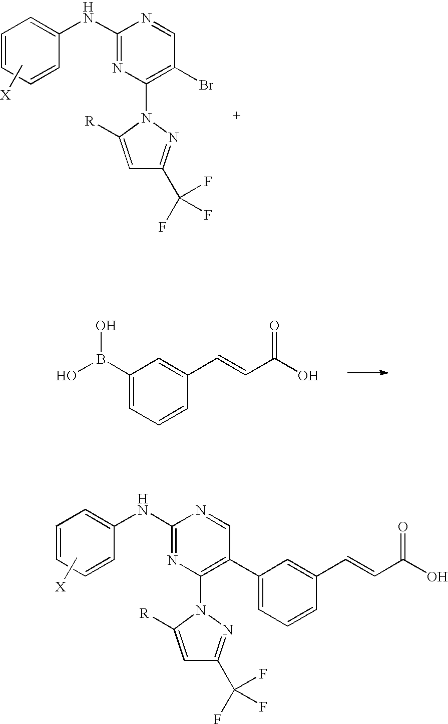

A suspension of 5-bromo-N-(aryl)-4-[3-(trifluoromethyl)-1H-pyrazol-1-yl]pyrimidin-2-amine or 5-bromo-N-(aryl)-4-[5-methyl-3-(trifluoromethyl)-1H-pyrazol-1-yl]pyrimidin-2-amine (1 eq), 3-(trans-2-carboxyvinyl)phenylboronic acid (1.1-1.5 eq), [1,1′-bis(diphenylphosphino)ferrocene]dichloropalladium(II) (20 mol %) and sodium carbonate (1.5-2 eq) in acetonitrile/water (4:1) was degassed and heated to 90° C. for 15-30 min under inert atmosphere. The solvent was removed in vacuo and the crude mixture taken in CHCl₃ was washed with water, brine, dried over Na₂SO₄, filtered and concentrated. The crude material was purified by silica gel column chromatography using chloroform:methanol (9:1) as eluent to give the product. The compounds in the below table were prepared using this procedure and the starting material indicated.

| | | Mass spectrum and ¹H | |
|---|---|---|---|
| Compound | Structure | NMR | SM |
| Example 688 | 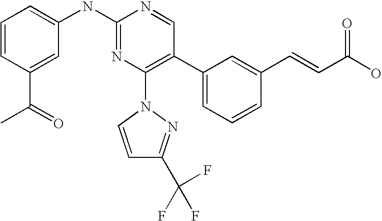 (2E)-3-(3-{2-[(3- acetylphenyl)amino]-4- [3-(trifluoromethyl)-1H- pyrazol-1-yl]pyrimidin- 5-yl}phenyl)prop-2-enoic acid | MS(ES): 494 (M + 1) for C₂₅H₁₈F₃N₅O₃. 400 MHz, DMSO-d₆: δ 2.60 (s, 3H), 6.50 (d, J = 16.04 Hz, 1H), 7.02 (d, J = 1.76 Hz, 1H), 7.17 (d, J = 7.48 Hz, 1H), 7.38 (t, J = 7.68 Hz, 1H), 7.54 (m, 3H), 7.65 (d, J = 6.80 Hz, 2H), 7.98 (d, J =8.04 Hz, 1H), 8.46 (s, 1H), 8.55 (s, 1H), 8.83 (s, 1H), 10.46 (s, 1H), 12.43 (s, 1H). | Intermediate 257 1-[3-({5- bromo-4-[3- (trifluoro- methyl)-1H- pyrazol-1- yl]pyrimidin- 2- yl}amino)- phenyl]ethanone |
| Example 689 | 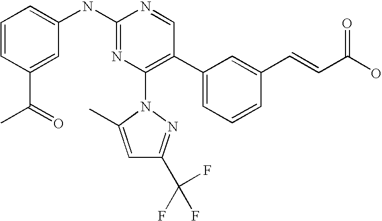 (2E)-3-(3-{2-[(3- acetylphenyl)amino]-4- [5-methyl-3- (trifluoromethyl)-1H- pyrazol-1-yl]pyrimidin- 5-yl}phenyl)prop-2-enoic acid | MS(ES): 508 (M + 1) for C₂₆H₂₀F₃N₅O₃. 400 MHz, DMSO-d₆: δ 2.24 (s, 3H), 2.56 (s, 3H), 6.44 (d, J = 16.04 Hz, 1H), 6.72 (s, 1H), 7.04 (d,J = 7.72 Hz, 1H), 7.33-7.37 (m, 2H), 7.48- 7.51 (m, 2H), 7.59-7.65 (m, 2H), 7.96 (d, J = 7.64 Hz, 1H), 8.43 (s, 1H), 8.97 (s, 1H), 10.46 (s, 1H), 12.43 (br s, 1H). | Intermediate 258 1-[3-({5- bromo-4-[5- methyl-3- (trifluoro- methyl)-1H- pyrazol-1- yl]pyrimidin- 2- yl}amino)- phenyl]ethanone |
| Example 690 | 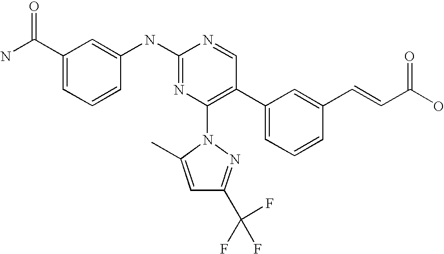 (2E)-3-(3-{2-[(3- carbamoylphenyl)amino]- 4-[3-(trifluoromethyl)- 1H-pyrazol-1- yl]pyrimidin-5- yl}phenyl)prop-2-enoic acid | MS(ES): 495 (M + 1) for C₂₄H₁₇F₃N₆O₃. 400 MHz, DMSO-d₆: δ 6.48 (d, J = 16.04 Hz, 1H), 7.00 (d, J = 2.44 Hz, 1H), 7.16 (d, J = 7.72 Hz, 1H), 7.34-7.43 (m, 4H), 7.53-7.62 (m, 3H), 7.86 (d, J = 8.24 Hz, 1H), 7.94 (br s, 1H), 8.38 (s, 1H), 8.51 (br s, 1H), 8.79 (s, 1H), 10.35 (s, 1H), 12.41 (s, 1H). | Intermediate 262 3-({5-bromo- 4-[3- (trifluoro- methyl)-1H- pyrazol-1- yl]pyrimidin- 2- yl}amino)- benzamide |
| Example 691 | 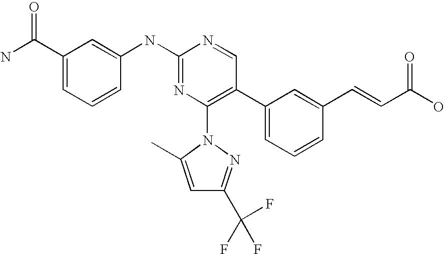 (2E)-3-(3-{2-[(3- carbamoylphenyl)amino]- 4-[5-methyl-3- (trifluoromethyl)-1H- pyrazol-1-yl]pyrimidin- 5-yl}phenyl)prop-2-enoic acid | MS(ES): 509 (M + 1) for C₂₅H₁₉F₃N₆O₃. 400 MHz, DMSO-d₆: δ 2.29 (s, 3H), 6.44 (d, J = 16.00 Hz, 1H), 6.72 (s, 1H), 7.03 (d, J = 7.76 Hz, 1H), 7.32-7.47 (m, 4H), 7.51-7.61 (m, 2H), 7.86 (d, J = 7.84 Hz, 1H), 7.93 (br s, 1H), 8.26 (s, 1H), 8.94 (s, 1H), 10.36 (s, 1H), 12.45 (br s, 1H). | Intermediate 263 3-({5-bromo- 4-[5-methyl- 3- (trifluoro- methyl)-1H- pyrazol-1- yl]pyrimidin- 2- yl}amino)- benzamide |
| Example 692^{c)} | 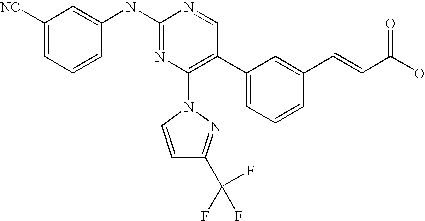 (2E)-3-(3-{2-[(3- cyanophenyl)amino]-4- [3-(trifluoromethyl)-1H- pyrazol-1-yl]pyrimidin- 5-yl}phenyl)prop-2-enoic acid | MS(ES): 477 (M + 1) for C₂₄H₁₅F₃N₆O₂. 400 MHz, DMSO-d₆: δ 6.48 (d, J = 16.00 Hz, 1H), 7.01 (d, J = 2.24 Hz, 1H), 7.14 (d, J = 7.56 Hz, 1H), 7.38 (t, J = 7.68 Hz, 1H), 7.47 (d, J = 7.56 Hz, 1H), 7.52-7.58 (m, 3H), 7.64 (d, J = 7.60 Hz, 1H), 8.05 (d, J = 8.08 Hz, 1H), 8.31 (s, 1H), 8.38 (s, 1H), 8.86 (s, 1H), 10.59 (s, 1H), 12.42 (s, 1H). | Intermediate 266 3-({5-bromo- 4-[3- (trifluoro- methyl)-1H- pyrazol-1- yl]pyrimidin- 2- yl}amino)- benzonitrile |
| Example 693^{c)} | 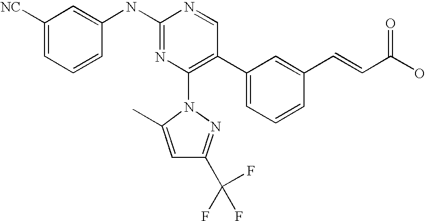 (2E)-3-(3-{2-[(3- cyanophenyl)amino]-4- [5-methyl-3- (trifluoromethyl)-1H- pyrazol-1-yl]pyrimidin- 5-yl}phenyl)prop-2-enoic acid | MS(ES): 491 (M + 1) for C₂₅H₁₇F₃N₆O₂. 400 MHz, DMSO-d₆: δ 2.26 (s, 3H), 6.45 (d, J = 16.00 Hz, 1H), 6.73 (s, 1H), 7.05 (d, J = 7.76 Hz, 1H), 7.34-7.36 (m, 2H), 7.47-7.62 (m, 4H), 8.00 (d, J = 8.12 Hz, 1H), 8.29 (s,1H), 9.02 (s, 1H), 10.63 (s, 1H), 12.45- 12.46 (m, 1H). | Intermediate 267 3-({5-bromo- 4-[5-methyl- 3- (trifluoro- methyl)-1H- pyrazol-1- yl]pyrimidin- 2- yl}amino)- benzonitrile |
| Example 694 | 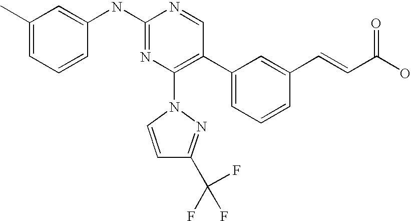 (2E)-3-(3-{2-[(3- methylphenyl)amino]-4- [3-(trifluoromethyl)-1H- pyrazol-1-yl]pyrimidin- 5-yl}phenyl)prop-2-enoic acid | MS(ES): 466 (M + 1) for C₂₄H₁₈F₃N₅O₂. 400 MHz, DMSO-d₆: δ 2.31 (s, 3H), 6.47 (d, J = 16.00 Hz, 1H), 6.85 (d, J = 7.56 Hz, 1H), 6.99 (d, J = 2.40 Hz, 1H), 7.14 (d, J = 7.96 Hz, 1H), 7.22 (t, J = 7.80 Hz, 1H), 7.36 (t, J = 7.80 Hz, 1H), 7.52 (m, 2H), 7.56-7.59 (m, 4H), 8.38 (s, 1H), 8.78 (s, 1H), 10.15 (s, 1H). | Intermediate 259 5-bromo-N- (3- methylphenyl)- 4-[3- (trifluoro- methyl)-1H- pyrazol-1- yl]pyrimidin- 2-amine |
| Example 695 | 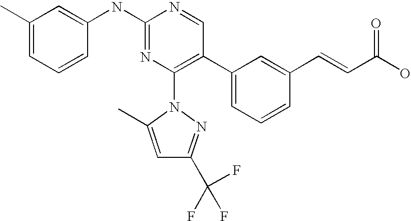 (2E)-3-(3-{2-[(3- methylphenyl)amino]-4- [5-methyl-3- (trifluoromethyl)-1H- pyrazol-1-yl]pyrimidin- 5-yl}phenyl)prop-2-enoic acid | MS(ES): 480 (M + 1) for C₂₅H₂₀F₃N₅O₂. 400 MHz, DMSO-d₆: δ 2.23 (s, 3H), 2.29 (s, 3H), 6.44 (s, 1H), 6.71 (s, 1H), 6.86 (d, J = 7.52 Hz, 1H), 7.04 (d, J = 8.04 Hz, 1H), 7.22 (t, J = 7.72 Hz, 1H), 7.33-7.36 (m, 2H), 7.49 (d, J = 16.00 Hz, 1H), 7.55-7.61 (m, 3H), 8.92 (s, 1H), 10.19 (s, 1H), 12.42 (s, 1H). | Intermediate 260 5-bromo-N- (3- methylphenyl)- 4-[5- methyl-3- (trifluoro- methyl)-1H- pyrazol-1- yl]pyrimidin- 2-amine |

^{c)}35 mol % XPHOS was also used.

### General method for the synthesis of ethyl 5-(2-{arylamino}-4-[1H-azol-1-yl]pyrimidin-5-yl)pyridine-3-carboxylate

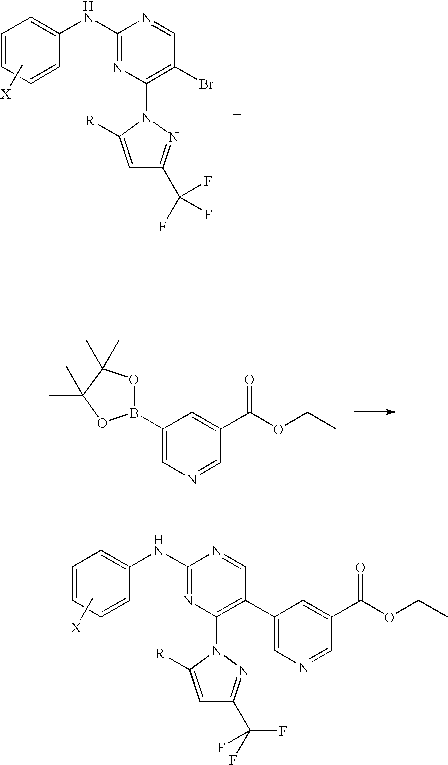

A suspension of 5-bromo-N-(aryl)-4-[3-(trifluoromethyl)-1H-pyrazol-1-yl]pyrimidin-2-amine or 5-bromo-N-(aryl)-4-[5-methyl-3-(trifluoromethyl)-1H-pyrazol-1-yl]pyrimidin-2-amine (1 eq), ethyl 5-(4,4,5,5-tetramethyl-1,3,2-dioxaborolan-2-yl)pyridine-3-carboxylate (1.1-1.2 eq), [1,1′-bis(diphenylphosphino)ferrocene]dichloropalladium(II) (10-20 mol %) and sodium carbonate (1-2 eq) in acetonitrile/water (4:1) was degassed and heated to 90° C. for 15-30 min under inert atmosphere. The solvent was removed in vacuo and the crude mixture taken in CHCl₃ was washed with water, brine, dried over Na₂SO₄, filtered and concentrated. The crude material was purified by silica gel column chromatography using 30% ethyl acetate/hexanes as eluent to give the product. The compounds in the below table were prepared using this method and the specified starting material.

| | | Mass spectrum and ¹H | |
|---|---|---|---|
| Compound | Structure | NMR | SM |
| Example 696 | 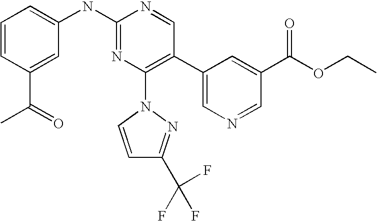 ethyl 5-{2-[(3- acetylphenyl)amino]-4- [3-(trifluoromethyl)-1H- pyrazol-1-yl]pyrimidin- 5-yl}pyridine-3- carboxylate | MS(ES): 497 (M + 1) for C₂₄H₁₉F₃N₆O₃. 400 MHz, DMSO-d₆: δ 1.30 (t, J = 7.12 Hz, 3H), 2.59 (s, 3H), 4.33 (q, J = 7.12 Hz, 2H), 7.07 (d, J = 2.56 Hz, 1H), 7.52 (t, J = 7.76 Hz, 1H), 7.67 (d, J = 7.36 Hz, 1H), 7.96 (d, J = 8.28 Hz, 1H), 8.07 (m, 1H), 8.52 (s, 1H), 8.65 (s, 1H), 8.70 (dd, J = 0.76, 2.14 Hz, 1H), 8.83 (d, J = 0.92 Hz, 1H), 9.02 (dd, J = 0.84, 1.92 Hz, 1H), 10.49 (s, 1H). | Intermediate 257 1-[3-({5- bromo-4-[3- (trifluoro- methyl)-1H- pyrazol-1- yl]pyrimidin- 2- yl}amino)- phenyl]ethanone |
| Example 697 | 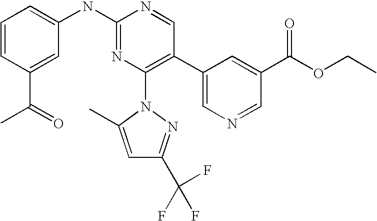 ethyl 5-{2-[(3- acetylphenyl)amino]-4- [5-methyl-3- (trifluoromethyl)-1H- pyrazol-1-yl]pyrimidin- 5-yl}pyridine-3- carboxylate | MS(ES): 511 (M + 1) for C₂₅H₂₁F₃N₆O₃. 400 MHz, DMSO-d₆: δ 1.29 (t, J = 7.08 Hz, 3H), 2.44 (s, 3H), 2.57 (s, 3H), 4.31 (q, J = 7.12 Hz, 2H), 6.78 (s, 1H), 7.51 (t, J = 7.84 Hz, 1H), 7.67 (d, J = 7.72 Hz, 1H), 7.80 (t, J = 1.96 Hz, 1H), 7.96 (d, J = 8.04 Hz, 1H), 8.41 (s, 1H), 8.61 (d, J = 2.08 Hz, 1H), 8.97 (d, J = 1.68 Hz, 1H), 9.01 (s, 1H), 10.50 (s, 1H). | Intermediate 258 1-[3-({5- bromo-4-[5- methyl-3- (trifluoro- methyl)-1H- pyrazol-1- yl]pyrimidin- 2- yl}amino)- phenyl]ethanone |
| Example 698 | 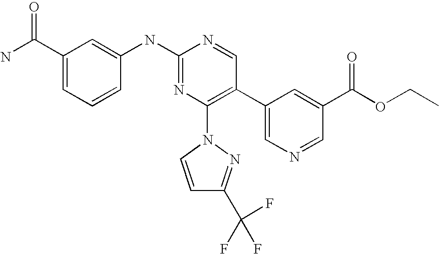 ethyl 5-{2-[(3- carbamoylphenyl)amino]- 4-[3-(trifluoromethyl)- 1H-pyrazol-1- yl]pyrimidin-5- yl}pyridine-3- carboxylate | MS(ES): 498 (M + 1) for C₂₃H₁₈F₃N₇O₃. 300 MHz, DMSO-d₆: δ 1.29 (t, J = 7.05 Hz, 3H), 4.32 (q, J = 7.05 Hz, 2H), 7.04 (d, J = 2.58 Hz, 1H), 7.38 (d, J = 4.77 Hz, 1H), 7.43 (d, J = 7.89 Hz, 1H), 7.54 (d, J = 7.86 Hz, 1H), 7.82 (d, J = 7.62 Hz, 1H), 7.96 (s, 1H), 8.06 (t, J = 2.04 Hz, 1H), 8.43 (s, 1H), 8.70 (d, J = 2.13 Hz, 2H), 8.79 (s, 1H), 9.02 (d, J = 1.92 Hz, 1H), 10.40 (s, 1H). | Intermediate 262 4-({5-bromo- 4-[3- (trifluoro- methyl)-1H- pyrazol-1- yl]pyrimidin- 2- yl}amino)- benzamide |
| Example 699 | 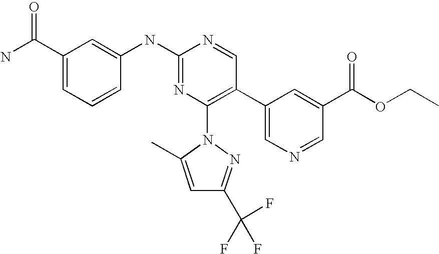 ethyl 5-{2-[(3- carbamoylphenyl)amino]- 4-[5-methyl-3- (trifluoromethyl)-1H- pyrazol-1-yl]pyrimidin- 5-yl}pyridine-3- carboxylate | MS(ES): 512 (M + 1) for C₂₄H₂₀F₃N₇O₃. 300 MHz, DMSO-d₆: δ 1.29 (t, J = 7.05 Hz, 3H), 2.46 (s, 3H), 4.30 (q, J = 6.99 Hz, 2H), 6.76 (s, 1H), 7.35 (br s, 1H), 7.41 (t, J = 8.13 Hz, 1H), 7.53 (d, J = 8.07 Hz, 1H), 7.78-7.85 (m, 2H), 7.93 (br s, 1H), 8.27 (s, 1H), 8.59 (d, J = 2.19 Hz, 1H), 8.97 (d, J = 3.06 Hz, 2H), 10.40 (s, 1H) | Intermediate 263 3-({5-bromo- 4-[5-methyl- 3- (trifluoro- methyl)-1H- pyrazol-1- yl]pyrimidin- 2- yl}amino)- benzamide |
| Example 700^{d)} | 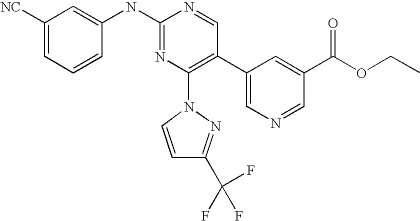 ethyl 5-{2-[(3- cyanophenyl)amino]-4- [3-(trifluoromethyl)-1H- pyrazol-1-yl]pyrimidin- 5-yl}pyridine-3- carboxylate | MS(ES): 480 (M + 1) for C₂₃H₁₆F₃N₇O₂. 400 MHz, DMSO-d₆: δ 1.30 (t, J = 7.04 Hz, 3H), 4.33 (q, J = 7.12 Hz, 2H), 7.07 (d, J = 2.60 Hz, 1H), 7.50 (dd, J = 1.04, 7.62 Hz, 1H), 7.59 (t, J = 8.12 Hz, 1H), 8.04-8.08 (m, 2H), 8.27 (s, 1H), 8.59 (s, 1H), 8.70 (m, 1H), 8.88 (s, 1H), 9.03-9.04 (m, 1H), 10.63 (s, 1H). | Intermediate 266 3-({5-bromo- 4-[3- (trifluoro- methyl)-1H- pyrazol-1- yl]pyrimidin- 2- yl}amino)- benzonitrile |
| Example 701^{d)} | 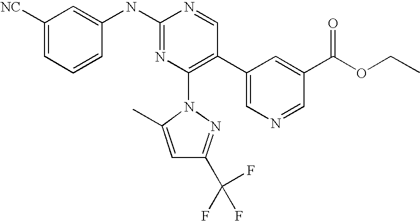 ethyl 5-{2-[(3- cyanophenyl)amino]-4- [5-methyl-3- (trifluoromethyl)-1H- pyrazol-1-yl]pyrimidin- 5-yl}pyridine-3- carboxylate | MS(ES): 494 (M + 1) for C₂₄H₁₈F₃N₇O₂. 400 MHz, DMSO-d₆: δ 1.31 (t, J = 7.08 Hz, 3H), 2.44 (s, 3H), 4.32 (q, J = 7.12 Hz, 2H), 6.81 (s, 1H), 7.50-7.52 (m, 1H), 7.59 (t, J = 8.04 Hz, 1H), 7.82 (t, J = 2.08 Hz, 1H), 8.00-8.02 (m, 1H), 8.29 (s, 1H), 8.63 (d, J = 2.20 Hz, 1H), 9.00 (d, J = 1.96 Hz, 1H), 9.06 (s, 1H), 10.67 (s, 1H). | Intermediate 267 3-({5-bromo- 4-[5-methyl- 3- (trifluoro- methyl)-1H- pyrazol-1- yl]pyrimidin- 2- yl}amino)- benzonitrile |
| Example 702 | 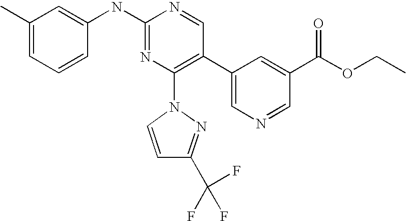 ethyl 5-{2-[(3- methylphenyl)amino]-4- [3-(trifluoromethyl)-1H- pyrazol-1-yl]pyrimidin- 5-yl}pyridine-3- carboxylate | Taken to the next step on the basis of LCMS. MS(ES): 469 (M + 1) for C₂₃H₁₉F₃N₆O₂. | Intermediate 259 5-bromo-N- (3- methylphenyl)- 4-[3- (trifluoro- methyl)-1H- pyrazol-1- yl]pyrimidin- 2-amine |
| Example 703 | 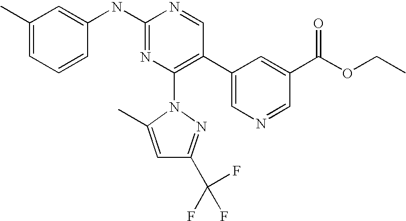 ethyl 5-{2-[(3- methylphenyl)amino]-4- [5-methyl-3- (trifluoromethyl)-1H- pyrazol-1-yl]pyrimidin- 5-yl}pyridine-3- carboxylate | MS(ES): 483 (M + 1) for C₂₄H₂₁F₃N₆O₂. 400 MHz, DMSO-d₆: δ 1.31 (t, J = 7.12 Hz, 3H), 2.31 (s, 3H), 2.43 (s, 3H), 4.28 (q, J = 7.08 Hz, 2H), 6.78 (s, 1H), 6.89 (d, J = 7.36 Hz, 1H), 7.24 (t, J = 7.72 Hz, 1H), 7.55 (s, 1H), 7.57 (s, 1H), 7.79 (t, J = 2.12 Hz, 1H), 8.61 (d, J = 2.24 Hz, 1H), 8.97 (s, 1H), 8.98 (d, J = 2.00 Hz, 1H), 10.25 (s, 1H). | Intermediate 260 5-bromo-N- (3- methylphenyl)- 4-[5- methyl-3- (trifluoro- methyl)-1H- pyrazol-1- yl]pyrimidin- 2-amine |

^{d)}35 mol % XPHOS was also used.

### General method for the synthesis of 5-{2-(arylamino)-4-[1H-pyrazol-1-yl]nyrimidin-5-yl}pyridine-3-carboxylic acid

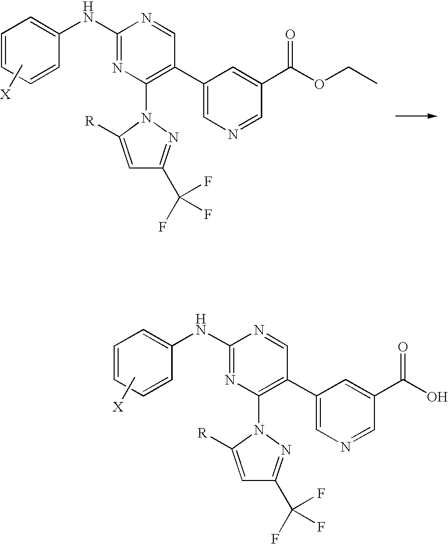

To 1 eq of ethyl 5-(2-{arylamino}-4-[1H-azol-1-yl]pyrimidin-5-yl)pyridine-3-carboxylate taken in dioxane and water, was added sodium hydroxide (2-2.5 eq) and stirred at RT for 4 h. The reaction mixture was carefully acidified with 1 N HCl and the solid that precipitated was filtered, washed with water and dried in vacuo to yield the desired carboxylic acid.

| | | Mass spectrum and ¹H | |
|---|---|---|---|
| Compound | Structure | NMR | SM |
| Example 704 | 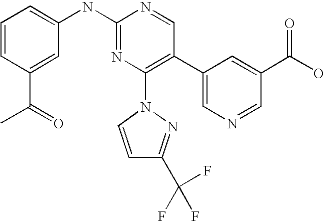 5-{2-[(3- acetylphenyl)amino]-4- [3-(trifluoromethyl)-1H- pyrazol-1-yl]pyrimidin- 5-yl}pyridine-3- carboxylic acid | MS(ES): 469 (M + 1) for C₂₂H₁₅F₃N₆O₃. 400 MHz, DMSO-d₆: δ 2.59 (s, 3H), 7.06 (d, J = 2.56 Hz, 1H), 7.52 (t, J = 7.84 Hz, 1H), 7.66 (d, J = 7.64 Hz, 1H), 7.97 (d, J = 7.88 Hz,1H), 8.05 (s, 1H), 8.52 (s, 1H), 8.64 (s, 1H), 8.66 (d, J = 2.08 Hz, 1H), 8.82 (s, 1H), 9.00 (d, J = 1.68 Hz, 1H), 10.48 (s, 1H),13.41 (br s, 1H). | Example 696 ethyl 5-{2- [(3- acetylphenyl) amino]-4-[3- (trifluoro- methyl)-1H- pyrazol-1- yl]pyrimidin- 5- yl}pyridine- 3-carboxylate |
| Example 705^{e)} | 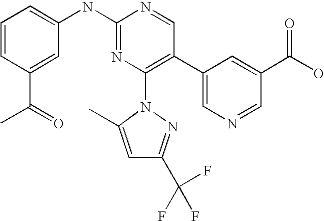 5-{2-[(3- acetylphenyl)amino]-4- [5-methyl-3- (trifluoromethyl)-1H- pyrazol-1-yl]pyrimidin- 5-yl}pyridine-3- carboxylic acid | MS(ES): 483 (M + 1) for C₂₃H₁₇F₃N₆O₃. 400 MHz, DMSO-d₆: δ 2.44 (s, 3H), 2.57 (s, 3H), 6.77 (s, 1H), 7.52 (t, J = 7.88 Hz, 1H), 7.67 (d, J = 7.80 Hz, 1H), 7.84 (t, J = 2.00 Hz, 1H), 7.97 (d, J = 6.84 Hz, 1H), 8.41 (s, 1H), 8.49 (s, 1H), 8.95 (d, J = 1.84 Hz, 1H), 8.98 (s, 1H), 10.49 (s, 1H),13.60 (br s, 1H). | Example 697 ethyl 5-{2- [(3- acetylphenyl) amino]-4-[5- methyl-3- (trifluoro- methyl)-1H- pyrazol-1- yl]pyrimidin- 5- yl}pyridine- 3-carboxylate |
| Example 706 | 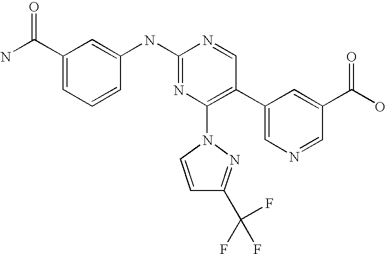 5-{2-[(3- carbamoylphenyl)amino]- 4-[3-(trifluoromethyl)- 1H-pyrazol-1- yl]pyrimidin-5- yl}pyridine-3-carboxylic acid | MS(ES): 470 (M + 1) for C₂₁H₁₄F₃N₇O₃. 400 MHz, DMSO-d₆: δ 7.04 (s, 1H), 7.38 (s, 1H), 7.43 (t, J = 7.84 Hz, 1H), 7.55 (d, J = 7.84 Hz, 1H), 7.84 (d, J = 7.76 Hz, 1H), 7.96 (s, 1H), 8.06 (s, 1H), 8.43 (s, 1H), 8.68 (d, J = 7.52 Hz, 2H), 8.79 (s, 1H), 9.01 (s, 1H), 10.39 (s, 1H). | Example 698 ethyl 5-{2- [(3- carbamoyl- phenyl)amino]- 4-[3- (trifluoro- methyl)-1H- pyrazol-1- yl]pyrimidin- 5- yl}pyridine- 3-carboxylate |
| Example 707^{f)} | 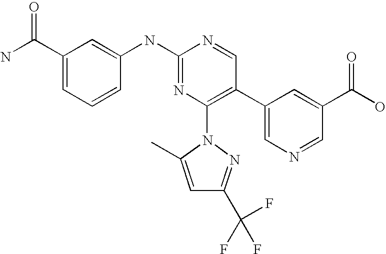 5-{2-[(3- carbamoylphenyl)amino]- 4-[5-methyl-3- (trifluoromethyl)-1H- pyrazol-1-yl]pyrimidin- 5-yl}pyridine-3- carboxylic acid | MS(ES): 484 (M + 1) for C₂₂H₁₆F₃N₇O₃. 400 MHz, DMSO-d₆: δ 2.48 (s, 3H), 6.76 (s, 1H), 7.36 (br s, 1H), 7.42 (t, J = 7.88 Hz, 1H), 7.54 (d, J = 7.60 Hz, 1H), 7.82-7.86 (m, 2H), 7.94 (s, 1H), 8.27 (s, 1H), 8.53 (d, J = 2.00 Hz, 1H), 8.95 (m, 2H), 10.40 (s, 1H), 13.47 (br s, 1H). | Example 699 ethyl 5-{2- [(3- carbamoyl- phenyl)amino]- 4-[5-methyl- 3- (trifluoro- methyl)-1H- pyrazol-1- yl]pyrimidin- 5- yl}pyridine- 3-carboxylate |
| Example 708^{f)} | 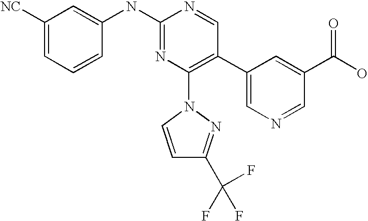 5-{2-[(3- cyanophenyl)amino]-4- [3-(trifluoromethyl)-1H- pyrazol-1-yl]pyrimidin- 5-yl}pyridine-3- carboxylic acid | MS(ES): 452 (M + 1) for C₂₁H₁₂F₃N₇O₂. 400 MHz, DMSO-d₆: δ 7.07 (d, J = 1.84 Hz, 1H), 7.50 (d, J = 7.48 Hz, 1H), 7.59 (t, J = 8.04 Hz, 1H), 8.04 (s, 1H), 8.08 (d, J = 8.16 Hz, 1H), 8.28 (s, 1H), 8.59 (s, 1H), 8.67 (s, 1H), 8.88 (s, 1H), 9.02 (s, 1H), 10.63 (s, 1H), 13.46 (br s, 1H). | Example 700 ethyl 5-{2- [(3- cyanophenyl) amino]-4-[3- (trifluoro- methyl)-1H- pyrazol-1- yl]pyrimidin- 5- yl}pyridine- 3-carboxylate |
| Example 709^{g)} | 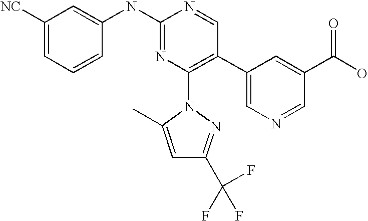 5-{2-[(3- cyanophenyl)amino]-4- [5-methyl-3- (trifluoromethyl)-1H- pyrazol-1-yl]pyrimidin- 5-yl}pyridine-3- carboxylic acid | MS(ES): 466 (M + 1) for C₂₂H₁₄F₃N₇O₂. 400 MHz, DMSO-d₆: δ 2.44 (s, 3H), 6.78 (s, 1H), 7.49 (d, J = 7.64 Hz, 1H), 7.57 (t, J = 8.08 Hz, 1H), 7.84 (d, J = 1.84 Hz, 1H), 8.00 (d, J = 8.40 Hz, 1H), 8.27 (s, 1H), 8.55 (s, 1H), 8.96 (s, 1H), 9.03 (s, 1H), 10.64 (s, 1H),13.46 (br s, 1H). | Example 701 ethyl 5-{2- [(3- cyanophenyl) amino]-4-[5- methyl-3- (trifluoro- methyl)-1H- pyrazol-1- yl]pyrimidin- 5- yl}pyridine- 3-carboxylate |
| Example 710^{g)} | 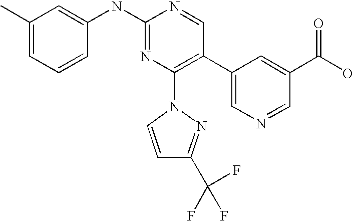 5-{2-[(3- methylphenyl)amino]-4- [3-(trifluoromethyl)-1H- pyrazol-1-yl]pyrimidin- 5-yl}pyridine-3- carboxylic acid | MS(ES): 441 (M + 1) for C₂₁H₁₅F₃N₆O₂. 400 MHz, DMSO-d₆: δ 2.33 (s, 3H),6.88 (d, J = 7.40 Hz, 1H), 7.04 (d, J = 2.64 Hz, 1H), 7.25 (t, J = 7.68 Hz, 1H), 7.60 (s, 1H), 7.62 (s, 1H), 8.02 (t, J = 2.04 Hz, 1H), 8.56 (s, 1H), 8.64 (s, 1H), 8.79 (s, 1H), 9.00 (d, J = 1.92 Hz, 1H), 10.21 (s, 1H), 13.44 (s, 1H). | Example 702 ethyl 5-{2- [(3- methylphenyl)- amino]-4-[3- (trifluoro- methyl)-1H- pyrazol-1- yl]pyrimidin- 5- yl}pyridine- 3-carboxylate |
| Example 711^{f)} | 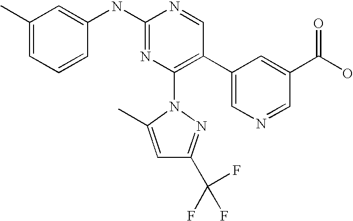 5-{2-[(3- methylphenyl)amino]-4- [5-methyl-3- (trifluoromethyl)-1H- pyrazol-1-yl]pyrimidin- 5-yl}pyridine-3- carboxylic acid | MS(ES): 455 (M + 1) for C₂₂H₁₇F₃N₆O₂. 400 MHz, DMSO-d₆: δ 2.31 (s, 3H), 2.44 (s, 3H), 6.77 (s, 1H),6.88 (d, J = 7.44 Hz, 1H), 7.24 (t, J = 8.12 Hz, 1H), 7.55 (s, 1H), 7.57 (s, 1H), 7.82 (t, J = 1.84 Hz,1H), 8.54 (d, J = 1.96 Hz, 1H), 8.95-8.96 (m, 2H), 10.23 (s, 1H), 13.42 (s, 1H). | Example 703 ethyl 5-{2- [(3- methylphenyl)- amino]-4-[5- methyl-3- (trifluoro- methyl)-1H- pyrazol-1- yl]pyrimidin- 5- yl}pyridine- 3-carboxylate |

^{e)}NaOH (2.5 eq), MeCN—H₂O, reflux, 15 min
^{f)}NaOH (2.5 eq), THF-H₂O, RT, 5 h
^{g)}Ba(OH)₂ (2 eq), dioxane-H₂O, RT.

### General method for the synthesis of methyl 5-(2-{arylaminol}-4-[1H-azol-1-yl]pyrimidin-5-yl)-2-methoxypyridine-3-carboxylate

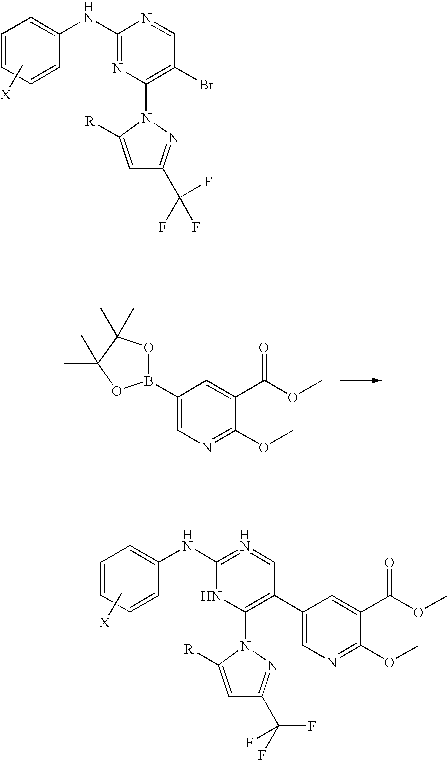

A suspension of 5-bromopyrimidine derivative (1 eq), methyl 2-methoxy-5-(4,4,5,5-tetramethyl-1,3,2-dioxaborolan-2-yl)pyridine-3-carboxylate (1.1-1.2 eq), [1,1′-bis(diphenylphosphino)ferrocene]-dichloropalladium(II) (10-20 mol %) and sodium carbonate (1-2 eq) in acetonitrile/water (4:1) was degassed and heated to 90° C. for 15-30 min under inert atmosphere. The solvent was removed in vacuo and the crude mixture taken in CHCl₃ was washed with water, brine, dried over Na₂SO₄, filtered and concentrated. The crude material was purified by silica gel column chromatography using 50% ethyl acetate/hexanes as eluent to give the product. The compounds in the below table were prepared using this method and the specified starting material.

| | | Mass spectrum and ¹H | |
|---|---|---|---|
| Compound | Structure | NMR | SM |
| Example 712 | 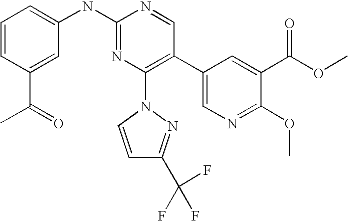 methyl 5-{2-[(3- acetylphenyl)amino]-4- [3-(trifluoromethyl)-1H- pyrazol-11-yl]pyrimidin- 5-yl}-2- methoxypyridine-3- carboxylate | MS(ES): 513 (M + 1) for C₂₄H₁₉F₃N₆O_{4.} 300 MHz, DMSO-d₆: δ 2.58 (s, 3H), 3.76 (s, 3H), 3.94 (s, 3H), 7.04 (s, 1H), 7.50 (t, J = 7.80 Hz, 1H), 7.65 (d, J = 7.95 Hz, 1H), 7.88 (d, J = 2.43 Hz, 1H), 7.95 (d, J = 8.04 Hz, 1H), 8.28 (d, J = 2.43 Hz, 1H), 8.51 (s, 1H), 8.59 (s, 1H), 8.78 (s, 1H), 10.41 (s, 1H). | Intermediate 257 1-[3-({5- bromo-4-[3- (trifluoro- methyl)-1H- pyrazol-1- yl]pyrimidin- 2- yl}amino)- phenyl]ethanone |
| Example 713 | 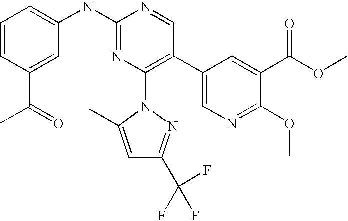 methyl 5-{2-[(3- acetylphenyl)amino]- [5-methyl-3- (trifluoromethyl)-1H- pyrazol-1-yl]pyrimidin- 5-yl}-2- methoxypyridine-3- carboxylate | MS(ES): 527 (M + 1) for C₂₅H₂₁F₃N₆O₄. 300 MHz, DMSO-d₆: δ 2.34 (s, 3H), 2.56 (s, 3H), 3.74 (s, 3H), 3.91 (s, 3H), 7.49 (t, J = 7.95 Hz, 1H), 7.60 (d, J = 2.31 Hz, 1H), 7.64 (d, J = 7.86 Hz, 1H), 7.95 (d, J = 8.10 Hz, 1H), 8.22 (d, J = 2.34 Hz, 1H), 8.30 (s, 1H), 8.40 (s, 1H), 8.95 (s, 1H), 10.44 (s, 1H). | Intermediate 258 1-[3-({5- bromo-4-[5- methyl-3- (trifluoro- methyl)-1H- pyrazol-1- yl]pyrimidin- 2- yl}amino)- phenyl]ethanone |
| Example 714 | 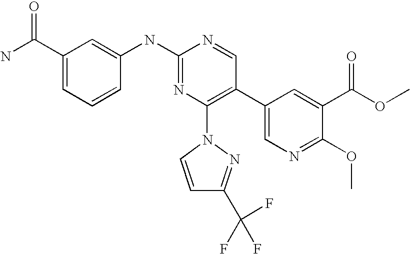 methyl 5-{2-[(3- carbamoylphenyl)amino]- 4-[3-(trifluoromethyl)- 1H-pyrazol-1- yl]pyrimidin-5-yl}-2- methoxypyridine-3- carboxylate | MS(ES): 514 (M + 1) for C₂₃H₁₈F₃N₇O₄. 300 MHz, DMSO-d₆: δ 3.76 (s, 3H), 3.93 (s, 3H), 7.02 (d, J = 2.55 Hz, 1H), 7.37 (d, J = 4.26 Hz, 1H), 7.42 (d, J = 7.71 Hz, 1H), 7.53 (d, J = 7.89 Hz, 1H), 7.82 (d, J = 8.58 Hz, 1H), 7.88 (d, J = 2.40 Hz, 1H), 7.95 (s, 1H), 8.28 (d, J = 2.46 Hz, 1H), 8.40 (s, 1H), 8.64 (s, 1H), 8.75 (s, 1H), 10.33 (s, 1H). | Intermediate 262 3-({5-bromo- 4-[3- (trifluoro- methyl)-1H- pyrazol-1- yl]pyrimidin- 2- yl}amino)- benzamide |
| Example 715 | 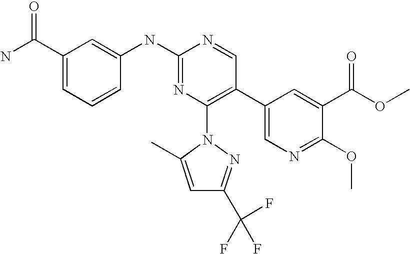 methyl 5-{2-[(3- carbamoylphenyl)amino]- 4-[5-methyl-3- (trifluoromethyl)-1H- pyrazol-1-yl]pyrimidin- 5-yl}-2- methoxypyridine-3- carboxylate | MS(ES): 528 (M + 1) for C₂₄H₂₀F₃N₇O₄. 400 MHz, DMSO-d₆: δ 2.39 (s, 3H), 3.75 (s, 3H), 3.91 (s, 3H), 6.68 (d, J = 8.28 Hz, 1H), 6.77 (s, 1H), 7.36 (br s, 1H), 7.42 (t, J = 7.76 Hz, 1H), 7.61 (d, J = 2.48 Hz, 1H), 7.85 (d, J = 8.68 Hz, 1H), 7.94 (br s, 1H), 8.23 (d, J = 2.48 Hz, 1H), 8.27 (s, 1H), 8.94 (s, 1H), 10.37 (s, 1H). | Intermediate 263 3-({5-bromo- 4-[5-methyl- 3- (trifluoro- methyl)-1H- pyrazol-1- yl]pyrimidin- 2- yl}amino)- benzamide |
| Example 716^{h)} | 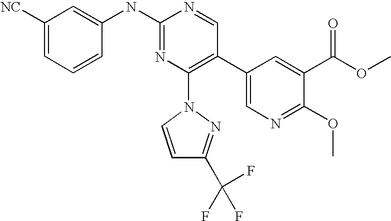 methyl 5-{2-[(3- cyanophenyl)amino]-4- [3-(trifluoromethyl)-1H- pyrazol-1-yl]pyrimidin- 5-yl}-2- methoxypyridine-3- carboxylate | MS(ES): 496 (M + 1) for C₂₃H₁₆F₃N₇O₃. 400 MHz, DMSO-d₆: δ 3.76 (s, 3H), 3.94 (s, 3H), 7.05 (d, J = 2.48 Hz, 1H), 7.46-7.49 (m, 1H), 7.57 (t, J = 8.16 Hz, 1H), 7.86 (d, J = 2.48 Hz, 1H), 8.04-8.06 (m, 1H), 8.27-8.31 (m, 2H), 8.53 (s, 1H), 8.83 (s, 1H), 10.57 (s, 1H). | Intermediate 266 3-({5-bromo- 4-[3- (trifluoro- methyl)-1H- pyrazol-1- yl]pyrimidin- 2- yl}amino)- benzonitrile |
| Example 717^{h)} | 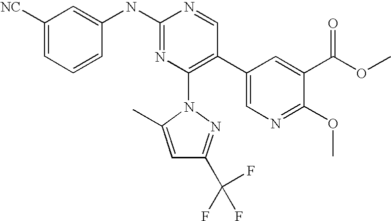 methyl 5-{2-[(3- cyanophenyl)amino]-4- [5-methyl-3- (trifluoromethyl)-1H- pyrazol-1-yl]pyrimidin- 5-yl}-2- methoxypyridine-3- carboxylate | MS(ES): 510 (M + 1) for C₂₄H₁₈F₃N₇O₃. 400 MHz, DMSO-d₆: δ 2.33 (s, 3H), 3.74 (s, 3H), 3.91 (s, 3H), 6.77 (s, 1H), 7.48 (dd, J = 1.16, 7.60 Hz, 1H), 7.56 (t, J = 8.12 Hz, 1H), 7.61 (d, J = 2.52 Hz, 1H), 7.97-8.00 (m, 1H), 8.23 (d, J = 2.52 Hz, 1H), 8.27 (s, 1H), 9.00 (s, 1H), 10.61 (s, 1H). | Intermediate 267 3-({5-bromo- 4-[5-methyl- 3- (trifluoro- methyl)-1H- pyrazol-1- yl]pyrimidin- 2- yl}amino)- benzonitrile |
| Example 718 | 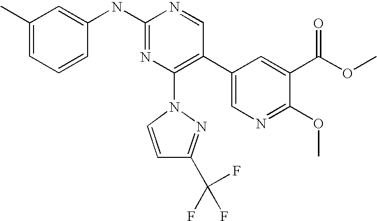 methyl 2-methoxy-5-{2- [(3- methylphenyl)amino]-4- [3-(trifluoromethyl)-1H- pyrazol-1-yl]pyrimidin- 5-yl}pyridine-3- carboxylate | Taken to the next step on the basis of LCMS. MS(ES): 485 (M + 1) for C₂₃H₁₉F₃N₆O₃. | Intermediate 259 5-bromo-N- (3- methylphenyl)- 4-[3- (trifluoro- methyl)-1H- pyrazol-1- yl]pyrimidin- 2-amine |
| Example 719 | 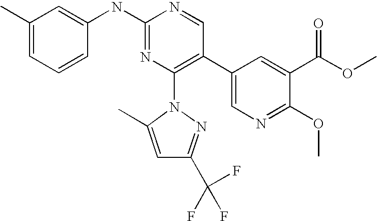 methyl 2-methoxy-5-{2- [(3- methylphenyl)amino]-4- [5-methyl-3- (trifluoromethyl)-1H- pyrazol-1-yl]pyrimidin- 5-yl}pyridine-3- carboxylate | MS(ES): 499 (M + 1) for C₂₄H₂₁F₃N₆O₃. 400 MHz, DMSO-d₆: δ 2.29 (s, 3H), 2.32 (s, 3H), 3.74 (s, 3H), 3.91 (s, 3H), 6.74 (s, 1H), 6.86 (d, J = 7.68 Hz, 1H), 7.22 (t, J = 8.80 Hz, 1H), 7.53-7.59 (m, 3H), 8.21 (d, J = 2.52 Hz, 1H), 8.90 (s, 1H), 10.18 (s, 1H). | Intermediate 260 5-bromo-N- (3- methylphenyl)- 4-[5- methyl-3- (trifluoro- methyl)-1H- pyrazol-1- yl]pyrimidin- 2-amine |

^{h)}35 mol % XPHOS was also used.

### General procedure for the synthesis of 5-{2-(arylamino)-4-[1H-pyrazol-1-yl]pyrimidin-5-yl}-2-methoxypyridine-3-carboxylic acid

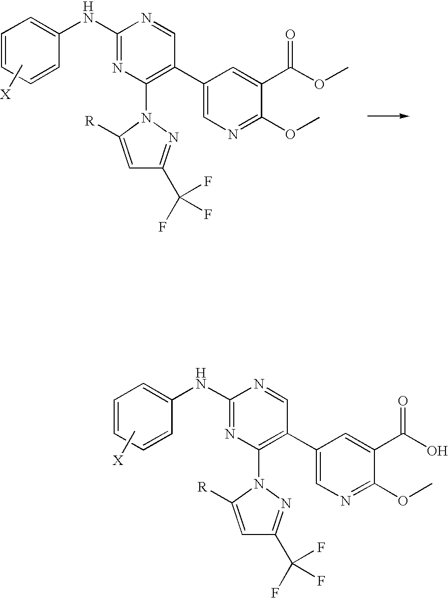

To 1 eq of methyl 5-(2-{arylamino}-4-[1H-azol-1-yl]pyrimidin-5-yl)-2-methoxypyridine-3-carboxylate taken in dioxane (5 mL), was added 1 N aq. sodium hydroxide or Barium hydroxide (2-6 eqs) and was heated to 60° C. for 1 h. The reaction mixture was carefully acidified with 1 N HCl and the solid that precipitated was filtered, washed with water and dried in vacuo to yield the desired carboxylic acid. The compounds in the below table were prepared using this method and the specified starting material.

| | | Mass spectrum and ¹H | |
|---|---|---|---|
| Compound | Structure | NMR | SM |
| Example 720 | 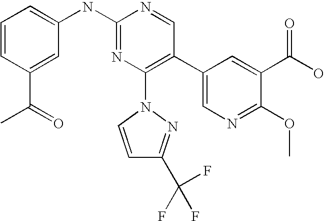 5-{2-[(3- acetylphenyl)amino]-4- [3-(trifluoromethyl)-1H- pyrazol-1-yl]pyrimidin- 5-yl}-2- methoxypyridine-3- carboxylic acid | MS(ES): 499 (M + 1) for C₂₃H₁₇F₃N₆O₄. 400 MHz, DMSO-d₆: δ 2.60 (s, 3H), 3.93 (s, 3H), 7.05 (d, J = 2.44 Hz, 1H), 7.52 (t, J = 7.88 Hz, 1H), 7.66 (d, J = 7.68 Hz, 1H), 7.83 (d,J = 2.04 Hz, 1H), 7.97 (d, J = 7.76 Hz, 1H), 8.22 (s, 1H), 8.53 (s, 1H), 8.58 (s, 1H), 8.79 (s, 1H), 10.43 (s, 1H), 12.96 (br s, 1H). | Example 713 methyl 5-{2- [(3- acetylphenyl) amino]-4-[3- (trifluoro- methyl)-1H- pyrazol-1- yl]pyrimidin- 5-yl}-2- methoxy- pyridine-3- carboxylate |
| Example 721 | 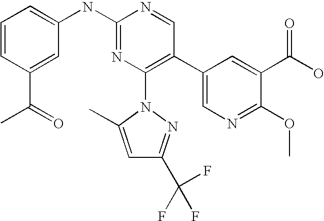 5-{2-[(3- acetylphenyl)amino]-4- [5-methyl-3- (trifluoromethyl)-1H- pyrazol-1-yl]pyrimidin- 5-yl}-2- methoxypyridine-3- carboxylic acid | MS(ES): 513 (M + 1) for C₂₄H₁₉F₃N₆O₄. 400 MHz, DMSO-d₆: δ 2.34 (s, 3H), 2.57 (s, 3H), 3.88 (s, 3H), 6.76 (s, 1H), 7.51 (t, J = 7.88 Hz, 1H), 7.58 (d, J = 2.28 Hz, 1H), 7.65 (d, J = 7.72 Hz, 1H), 7.97 (d, J = 7.56 Hz, 1H), 8.04 (s, 1H), 8.42 (s, 1H), 8.94 (s, 1H), 10.45 (s, 1H), 13.19 (s, 1H). | Example 714 methyl 5-{2- [(3- acetylphenyl) amino]-4-[5- methyl-3- (trifluoro- methyl)-1H- pyrazol-1- yl]pyrimidin- 5-yl}-2- methoxy- pyridine-3- carboxylate |
| Example 722ⁱ⁾ | 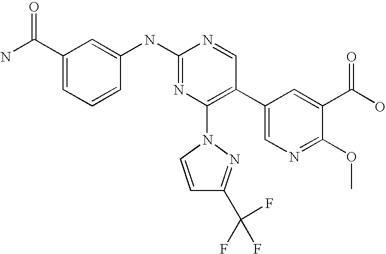 5-{2-[(3- carbamoylphenyl)amino]- 4-[3-(trifluoromethyl)- 1H-pyrazol-1- yl]pyrimidin-5-yl}-2- methoxypyridine-3- carboxylic acid | MS(ES): 500 (M + 1) for C₂₂H₁₆F₃N₇O₄. 400 MHz, DMSO-d₆: δ 3.93 (s, 3H), 7.03 (s, 1H), 7.38-7.43 (m, 2H), 7.53 (d, J = 7.52 Hz, 1H), 7.82-7.85 (m, 2H), 7.96 (s, 1H), 8.25 (s, 1H), 8.40 (s, 1H), 8.63 (s, 1H), 8.75 (s, 1H), 10.34 (s, 3H), 12.91 (br s, 1H). | Example 715 methyl 5-{2- [(3- carbamoyl- phenyl)amino]- 4-[3- (trifluoro- methyl)-1H- pyrazol-1- yl]pyrimidin- 5-yl}-2- methoxy- pyridine-3- carboxylate |
| Example 723ⁱ⁾ | 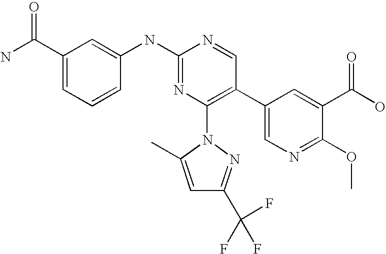 5-{2-[(3- carbamoylphenyl)amino]- 4-[5-methyl-3- (trifluoromethyl)-1H- pyrazol-1-yl]pyrimidin- 5-yl}-2- methoxypyridine-3- carboxylic acid | MS(ES): 514 (M + 1) for C₂₃H₁₈F₃N₇O₄. 400 MHz, DMSO-d₆: δ 2.39 (s, 3H), 3.89 (s, 3), 6.74 (s, 1H), 7.35 (br s, 1H), 7.40 (t, J = 7.88 Hz, 1H), 7.52 (d, J = 7.80 Hz, 1H), 7.61 (d, J = 2.52 Hz, 1H), 7.84 (d, J = 7.84 Hz, 1H), 7.93 (br s, 1H), 8.15 (d, J = 2.44 Hz, 1H), 8.25 (t, J = 1.76 Hz, 1H), 8.91 (s, 1H), 10.34 (s, 1H), 12.96 (br s, 1H). | Example 716 methyl 5-{2- [(3- carbamoyl- phenyl)amino]- 4-[5-methyl- 3- (trifluoro- methyl)-1H- pyrazol-1- yl]pyrimidin- 5-yl}-2- methoxy- pyridine-3- carboxylate |
| Example 724^{j)} | 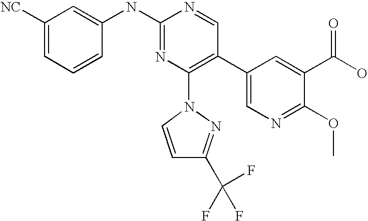 5-{2-[(3- cyanophenyl)amino]-4- [3-(trifluoromethyl)-1H- pyrazol-1-yl]pyrimidin- 5-yl}-2- methoxypyridine-3- carboxylic acid | MS(ES): 482 (M + 1) for C₂₂H₁₄F₃N₇O₃. 400 MHz, DMSO-d₆: δ 3.93 (s, 3H), 7.04 (d, J = 2.52 Hz, 1H), 7.48 (d, J = 7.64 Hz, 1H), 7.57 (t, J = 8.12 Hz, 1H), 7.83 (d, J = 2.48 Hz, 1H), 8.05 (d, J = 7.68 Hz, 1H), 8.25 (d, J = 2.44 Hz, 1H), 8.28 (s, 1H), 8.53 (s, 1H), 8.83 (s, 1H), 10.57 (s, 1H), 12.96 (br s, 1H). | Example 717 methyl 5-{2- [(3- cyanophenyl) amino]-4-[3- (trifluoro- methyl)-1H- pyrazol-1- yl]pyrimidin- 5-yl}-2- methoxy- pyridine-3- carboxylate |
| Example 725^{j)} | 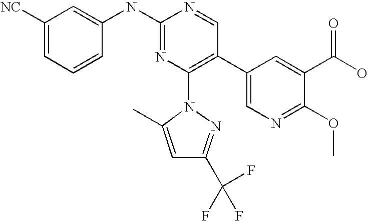 5-{2-[(3- cyanophenyl)amino]-4- [5-methyl-3- (trifluoromethyl)-1H- pyrazol-1-yl]pyrimidin- 5-yl}-2- methoxypyridine-3- carboxylic acid | MS(ES): 496 (M + 1) for C₂₃H₁₆F₃N₇O₃. 400 MHz, DMSO-d₆: δ 2.34 (s, 3H), 3.90 (s, 3H), 6.77 (s, 1H), 7.48 (d, J = 7.52 Hz, 1H), 7.57 (t, J = 8.12 Hz, 1H), 7.62 (d, J = 2.08 Hz, 1H), 7.99 (d, J = 8.80 hz, 1H), 8.16 (d, J = 1.76 Hz, 1H), 8.27 (s, 1H), 9.00 (s, 1H), 10.60 (s, 1H), 13.00 (br s, 1H). | Example 718 methyl 5-{2- [(3- cyanophenyl) amino]-4-[5- methyl-3- (trifluoro- methyl)-1H- pyrazol-1- yl]pyrimidin- 5-yl}-2- methoxy- pyridine-3- carboxylate |
| Example 726 | 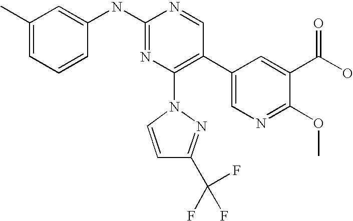 2-methoxy-5-{2-[(3- methylphenyl)amino]-4- [3-(trifluoromethyl)-1H- pyrazol-1-yl]pyrimidin- 5-yl}pyridine-3- carboxylic acid | MS(ES): 471 (M + 1) for C₂₂H₁₇F₃N₆O₃. 400 MHz, DMSO-d₆: δ 2.31 (s, 3H), 3.92 (s, 3H), 6.85 (d, J = 7.68 Hz, 1H), 7.01 (s, 1H), 7.23 (t, J = 8.16 Hz, 1H), 7.58 (s, 1H), 7.60 (s, 1H), 7.81 (s, 1H), 8.22 (s, 1H), 8.50 (s, 1H), 8.74 (s, 1H), 10.13 (s, 1H), 12.92 (s, 1H). | Example 719 methyl 2- methoxy-5- {2-[(3- methylphenyl)- amino]-4-[3- (trifluoro- methyl)-1H- pyrazol-1- yl]pyrimidin- 5- yl}pyridine- 3-carboxylate |
| Example 727^{k)} | 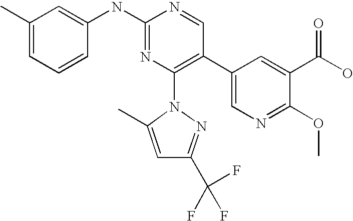 2-methoxy-5-{2-[(3- methylphenyl)amino]-4- [5-methyl-3- (trifluoromethyl)-1H- pyrazol-1-yl]pyrimidin- 5-yl}pyridine-3- carboxylic acid | MS(ES): 485 (M + 1) for C₂₃H₁₉F₃N₆O₃. 400 MHz, DMSO-d₆: δ 2.29 (s, 3H), 2.34 (s, 3H), 3.90 (s, 3H), 6.74 (s, 1H), 6.86 (d, J = 7.52 Hz, 1H), 7.22 (t, J = 8.68 Hz, 1H), 7.54-7.55 (m, 2H), 7.61 (d, J = 2.52 Hz, 1H), 8.15 (d, J = 2.48 Hz, 1H), 8.89 (s, 1H), 10.16 (s, 1H), 12.95 (s, 1H). | Example 720 methyl 2- methoxy-5- {2-[(3- methylphenyl)- amino]-4-[5- methyl-3- (trifluoro- methyl)-1H- pyrazol-1- yl]pyrimidin- 5- yl}pyridine- 3-carboxylate |

ⁱ⁾NaOH (2 eq), THF-H₂O, RT, 5 h
^{j)}Ba(OH)₂ (2 eq), dioxane-H₂O, RT
^{k)}NaOH (4 eq), THF-H₂O, RT, 4 h.

### Example 728

### (2E)-3-(3-{2-[(3-cyano-5-methoxyphenyl)amino]-4-[3-(trifluoromethyl)-1H-pyrazol-1-yl]pyrimidin-5-yl}phenyl)prop-2-enoic acid

### Example 729

### (2E)-3-(3-{2-[(3-cyano-5-methoxyphenyl)amino]-4-[5-methyl-3-(trifluoromethyl)-1H-pyrazol-1-yl]pyrimidin-5-yl}phenyl)prop-2-enoic acid

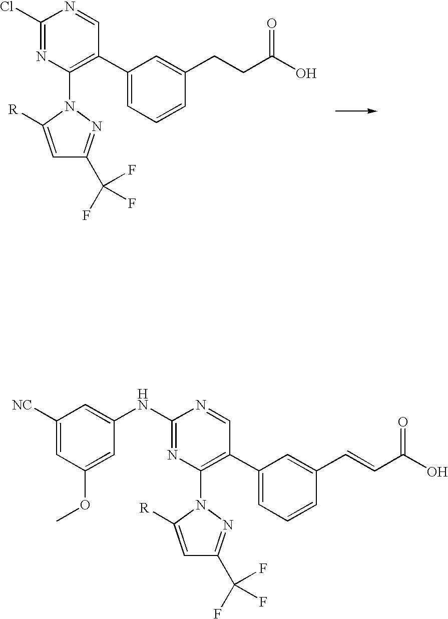

A suspension of (2E)-3-(3-{2-chloro-4-[3-(trifluoromethyl)-1H-pyrazol-1-yl]pyrimidin-5-yl}phenyl)prop-2-enoic acid (Intermediate 268, 1 eq) or (2E)-3-(3-{2-chloro-4-[5-methyl-3-(trifluoromethyl)-1H-pyrazol-1-yl]pyrimidin-5-yl}phenyl)prop-2-enoic acid (Intermediate 269, 1 eq), 3-amino-5-methoxybenzonitrile (1 eq), tris(dibenzylideneacetone)dipalladium(0) (10 mol %), 2-dicyclohexylphosphino-2′,4′,6′-triisopropyl-1,1′-biphenyl (20 mol %) and sodium carbonate (2 eq) in acetonitrile/water (5:1) was heated to 90° C. for 30 min. The reaction mixture was concentrated, the residue was taken in ethyl acetate and washed with water and brine. The organic layer was separated, dried over sodium sulfate, filtered and concentrated. The crude mass was purified by silica gel column chromatography (60-120 mesh) using MeOH/CHCl₃ as an eluent to give the product. The compounds in the below table were prepared using this method and the specified starting material.

| | | Mass spectrum and | |
|---|---|---|---|
| Compound | Structure | ¹H NMR | SM |
| Example 728 | 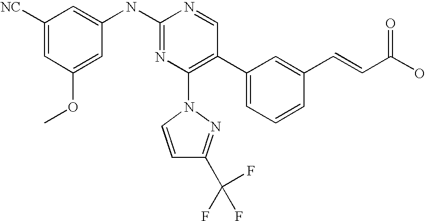 (2E)-3-(3-{2-[(3-cyano-5- methoxyphenyl)amino]-4-[3- (trifluoromethyl)-1H- pyrazol-1-yl]pyrimidin-5- yl}phenyl)prop-2-enoic acid | MS(ES): 507 (M + 1) for C₂₅H₁₇F₃N₆O₃. 400 MHz, DMSO-d₆: δ 3.82 (s, 3H), 6.48 (d, J = 16.04 Hz, 1H), 7.00 (d, J = 2.56 Hz, 1H), 7.09 (t, J = 1.80 Hz, 1H), 7.14 (d, J = 7.96 Hz, 1H), 7.38 (t, J = 7.72 Hz, 1H), 7.52-7.56 (m, 2H), 7.64 (d, J = 7.72 Hz, 1H), 7.83 (m, 2H), 8.32 (d, J = 1.72 Hz, 1H), 8.87 (s, 1H), 10.56 (s, 1H), 12.43 (br s, 1H). | Intermediate 268 (2E)-3-(3-{2- chloro-4-[3- (trifluoro- methyl)-1H- pyrazol-1- yl]pyrimidin- 5- yl}phenyl)- prop-2-enoic acid |
| Example 729 | 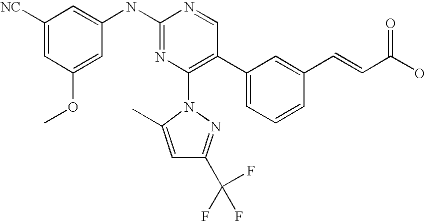 (2E)-3-(3-{2-[(3-cyano-5- methoxyphenyl)amino]-4-[5- methyl-3-(trifluoromethyl)- 1H-pyrazol-1-yl]pyrimidin- 5-yl}phenyl)prop-2-enoic acid | MS(ES): 521 (M + 1) for C₂₆H₁₉F₃N₆O₃. 400 MHz, DMSO-d₆: δ 2.21 (s, 3H), 3.81 (s, 3H), 6.45 (d, J = 16.04 Hz, 1H), 6.74 (s, 1H), 7.06 (d, J = 7.88 Hz, 1H), 7.11 (dd, J = 1.24, 2.16 Hz, 1H), 7.35-7.39 (m, 2H), 7.51 (d, J = 16.04 Hz, 1H), 7.63 (d, J = 7.88 Hz, 1H), 7.75 (t, J = 2.12 Hz, 1H), 7.83 (s, 1H), 9.04 (s, 1H), 10.61 (s, 1H), 12.46 (br s, 1H). | Intermediate 269 (2E)-3-(3-{2- chloro-4-[5- methyl-3- (trifluoro- methyl)-1H- pyrazol-1- yl]pyrimidin- 5- yl}phenyl)- prop-2-enoic acid |

### Example 730

### ethyl 5-{2-[(3-cyano-5-methoxyphenyl)amino]-4-[3-(trifluoromethyl)-1H-pyrazol-1-yl]pyrimidin-5-yl}pyridine-3-carboxylate

### Example 731

### ethyl 5-{2-[(3-cyano-5-methoxyphenyl)amino]-4-[5-methyl-3-(trifluoromethyl)-1H-pyrazol-1-yl]pyrimidin-5-yl}pyridine-3-carboxylate

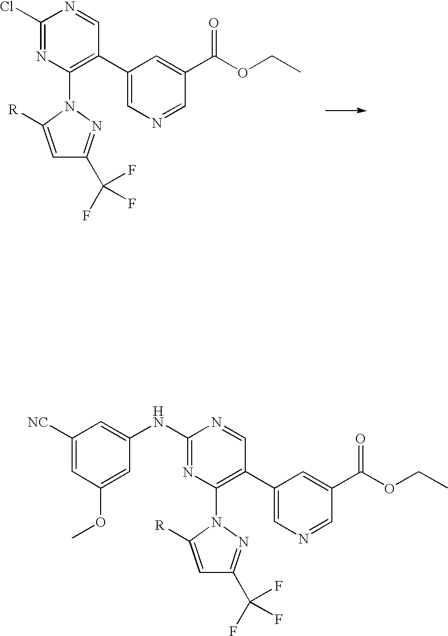

A suspension of ethyl 5-{2-chloro-4-[3-(trifluoromethyl)-1H-pyrazol-1-yl]pyrimidin-5-yl}pyridine-3-carboxylate (Intermediate 270, 1 eq) or ethyl 5-{2-chloro-4-[5-methyl-3-(trifluoromethyl)-1H-pyrazol-1-yl]pyrimidin-5-yl}pyridine-3-carboxylate (Intermediate 271, 1 eq), 3-amino-5-methoxybenzonitrile (1.2 eq), tris(dibenzylideneacetone)dipalladium(0) (10 mol %), 2-dicyclohexylphosphino-2′,4′,6′-triisopropyl-1,1′-biphenyl (20 mol %) and sodium carbonate (1 eq) in acetonitrile/water (5:1) was heated to 90° C. for 30 minutes. The reaction mixture was concentrated. The residue taken in ethyl acetate was washed with water and brine. The organic layer was separated, dried over sodium sulfate, filtered and concentrated. The crude mass was purified by silica gel column chromatography (60-120 mesh) using ethyl acetate/hexanes as an eluent to give the product. The compounds in the below table were prepared using this method and the specified starting material.

| | | Mass spectrum and | |
|---|---|---|---|
| Compound | Structure | ¹H NMR | SM |
| Example 730 | 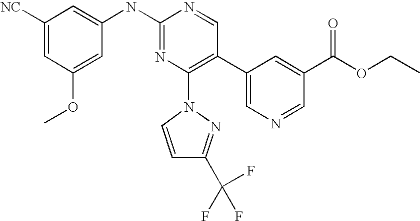 ethyl 5-{2-[(3-cyano-5- methoxyphenyl)amino]-4-[3- (trifluoromethyl)-1H- pyrazol-1-yl]pyrimidin-5- yl}pyridine-3-carboxylate | MS(ES): 510 (M + 1) for C₂₄H₁₈F₃N₇O_{3.} 400 MHz, DMSO-d₆: δ 1.29 (t, J = 7.08 Hz, 3H), 3.82 (s, 3H), 4.32 (q, J = 7.08 Hz, 2H), 7.06 (d, J = 2.58 Hz, 1H), 7.11 (t, J = 1.26 Hz, 1H), 7.78 (d, J = 2.22 Hz, 1H), 7.82 (s, 1H), 8.04 (t, J = 2.13 Hz, 1H), 8.54 (d, J = 1.62 Hz, 1H), 8.69 (d, J = 2.19 Hz, 1H), 8.88 (s, 1H), 9.03 (d, J = 1.98s Hz, 1H), 10.59 (s, 1H). | Intermediate 270 ethyl 5-{2- chloro-4-[3- (trifluoro- methyl)-1H- pyrazol-1- yl]pyrimidin- 5-yl}pyridine- 3-carboxylate |
| Example 731 | 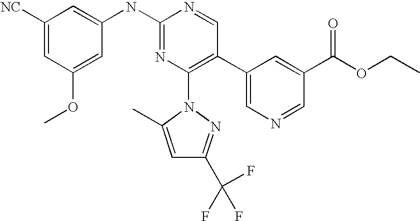 ethyl 5-{2-[(3-cyano-5- methoxyphenyl)amino]-4-[5- methyl-3-(trifluoromethyl)- 1H-pyrazol-1-yl]pyrimidin- 5-yl}pyridine-3-carboxylate | MS(ES): 524 (M + 1) for C₂₅H₂₀F₃N₇O₃. 400 MHz, DMSO-d₆: δ 1.30 (t, J = 7.04 Hz, 3H), 2.41 (s, 3H), 3.81 (s, 3H), 4.31 (q, J = 6.92 Hz, 2H), 6.79 (s, 1H), 7.12 (d, J = 1.16 Hz, 1H), 7.71 (d, J = 1.88 Hz, 1H), 7.82 (d, J = 1.96 Hz, 1H), 7.83 (s, 1H), 8.62 (s, 1H), 8.99 (s, 1H), 9.05 (s, 1H), 10.61 (s, 1H). | Intermediate 271 ethyl 5-{2- chloro-4-[5- methyl-3- (trifluoro- methyl)-1H- pyrazol-1- yl]pyrimidin- 5-yl}pyridine- 3-carboxylate |

### Example 732

### 5-{2-[(3-cyano-5-methoxyphenyl)amino]-4-[3-(trifluoromethvl)-1H-pyrazol-1-yl]pyrimidin-5-yl}pyridine-3-carboxylic acid

### Example 733

### 5-{2-[(3-cyano-5-methoxyphenyl)amino]-4-[5-methyl-3-(trifluoromethyl)-1H-pyrazol-1-yl]pyrimidin-5-yl}pyridine-3-carboxylic acid

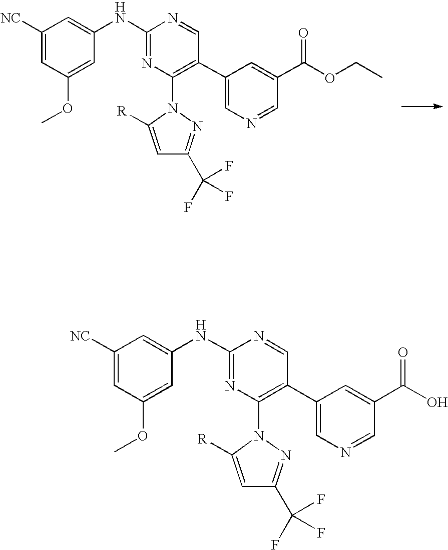

To ethyl 5-{2-[(3-cyano-5-methoxyphenyl)amino]-4-[3-(trifluoromethyl)-1H-pyrazol-1-yl]pyrimidin-5-yl}pyridine-3-carboxylate (Example 730, 1 eq) or ethyl 5-{2-[(3-cyano-5-methoxyphenyl)amino]-4-[5-methyl-3-(trifluoromethyl)-1H-pyrazol-1-yl]pyrimidin-5-yl}pyridine-3-carboxylate (Example 731, 1 eq) taken in a mixture of dioxane (5 mL) and water (5 mL), was added sodium hydroxide (2.5 eq) and stirred at room temperature for 3-5 h. The reaction mixture was carefully acidified with 1 N HCl and further extracted with ethyl acetate (50 mL). The organic layer was washed with water and brine, dried over Na₂SO₄ and concentrated to give the product. The compounds in the below table were prepared using this method and the specified starting material.

| | | Mass spectrum and | |
|---|---|---|---|
| Compound | Structure | ¹H NMR | SM |
| Example 732 | 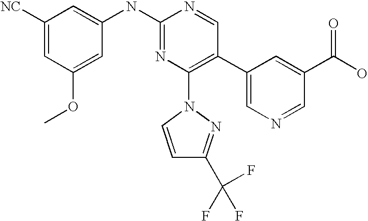 5-{2-[(3-cyano-5- methoxyphenyl)amino]-4-[3- (trifluoromethyl)-1H- pyrazol-1-yl]pyrimidin-5- yl}pyridine-3-carboxylic acid | MS(ES): 482 (M + 1) for C₂₂H₁₄F₃N₇O₃. 400 MHz, DMSO-d₆: δ 3.83 (s, 3H), 7.07 (d, J = 2.60 Hz, 1H), 7.12 (dd, J = 1.32, 2.18 Hz, 1H), 7.80 (t, J = 2.00 Hz, 1H), 7.83 (s, 1H), 8.03 (t, J = 2.04 Hz, 1H), 8.53 (d, J = 1.64 Hz, 1H), 8.62 (s, 1H), 8.89 (s, 1H), 9.01 (d, J = 1.84 Hz, 1H), 10.60 (s, 1H), 13.53 (s, 1H). | Example 730 ethyl 5-{2-[(3- cyano-5- methoxy- phenyl)amino]- 4-[3-(tri- fluoromethyl)-1H- pyrazol-1- yl]pyrimidin- 5-yl}pyridine- 3-carboxylate |
| Example 733 | 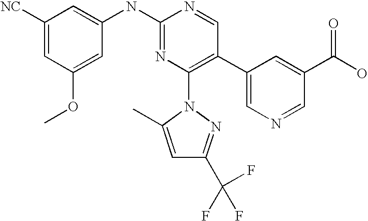 5-{2-[(3-cyano-5- methoxyphenyl)amino]-4-[5- methyl-3-(trifluoromethyl)- 1H-pyrazol-1-yl]pyrimidin- 5-yl}pyridine-3-carboxylic acid | MS(ES): 496 (M + 1) for C₂₃H₁₆F₃N₇O₃. 400 MHz, DMSO-d₆: δ 2.43 (s, 3H), 3.82 (s, 3H), 6.80 (s, 1H), 7.14 (dd, J = 1.28, 2.16 Hz, 1H), 7.71 (t, J = 2.12 Hz, 1H), 7.85 (t, J = 2.16 Hz, 2H), 8.56 (d, J = 2.20 Hz, 1H), 8.98 (s, 1H), 9.05 (s, 1H), 10.61 (s, 1H). | Example 731 ethyl 5-{2-[(3- cyano-5- methoxyphenyl)- amino]-4-[5- methyl-3- (trifluoro- methyl)-1H- pyrazol-1- yl]pyrimidin- 5-yl}pyridine- 3-carboxylate |

### Example 734

### methyl 5-{2-[(3-cyano-5-methoxyphenyl)amino]-4-[3-(trifluoromethyl)-1H-pyrazol-1-yl]pyrimidin-5-yl}-2-methoxypyridine-3-carboxylate

### Example 735

### methyl 5-{2-[(3-cyano-5-methoxyphenyl)amino]-4-[5-methyl-3-(trifluoromethyl)-1H-pyrazol-1-yl]pyrimidin-5-yl}-2-methoxypyridine-3-carboxylate

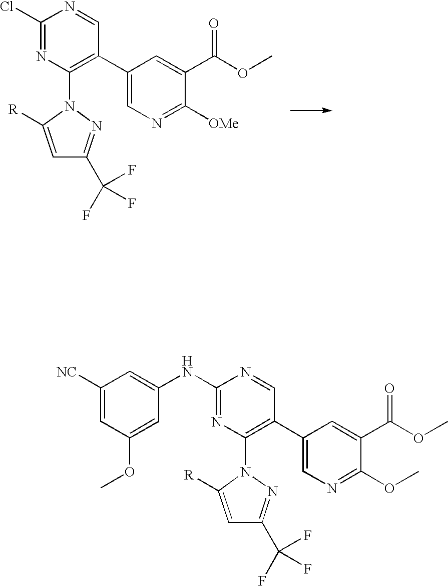

A suspension of methyl 5-{2-chloro-4-[3-(trifluoromethyl)-1H-pyrazol-1-yl]pyrimidin-5-yl}-2-methoxypyridine-3-carboxylate (Intermediate 325, 1 eq) or methyl 5-{2-chloro-4-[5-methyl-3-(trifluoromethyl)-1H-pyrazol-1-yl]pyrimidin-5-yl}-2-methoxypyridine-3-carboxylate (Intermediate 273, 1 eq), 3-amino-5-methoxybenzonitrile (1.0 eq), tris(dibenzylideneacetone)dipalladium(0) (10 mol %), 2-dicyclohexylphosphino-2′,4′,6′-triisopropyl-1,1′-biphenyl (20 mol %) and sodium carbonate (1 eq) in acetonitrile/water (5:1) was heated to 90° C. for 30 min. The reaction mixture was concentrated. The residue was taken in ethyl acetate, washed with water and brine. The organic layer was separated, dried over sodium sulfate, filtered and concentrated. The crude mass was purified by silica gel column chromatography (60-120 mesh) using ethyl acetate/hexanes as an eluent to give the product. The compounds in the below table were prepared using this method and the specified starting material.

| | | Mass spectrum and | |
|---|---|---|---|
| Compound | Structure | ¹H NMR | SM |
| Example 734 | 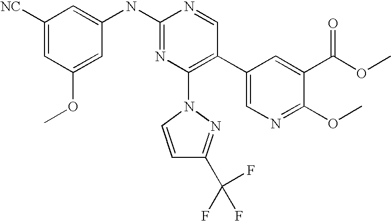 methyl 5-{2-[(3-cyano-5- methoxyphenyl)amino]-4-[3- (trifluoromethyl)-1H- pyrazol-1-yl]pyrimidin-5- yl}-2-methoxypyridine-3- carboxylate | MS(ES): 526 (M + 1) for C₂₄H₁₈F₃N₇O₄. 400 MHz, DMSO-d₆: δ 33.77 (s, 3H), 3.83 (s, 3H), 3.95 (s, 3H), 7.06 (d, J = 2.68 Hz, 1H), 7.11 (dd, J = 1.28, 2.30 Hz, 1H), 7.79 (t, J = 1.80 Hz, 1H), 7.83 (s, 1H), 7.87 (d, J = 2.48 Hz, 1H), 8.29 (d, J = 2.48 Hz, 1H), 8.50 (t, J = 1.76 Hz, 1H), 8.85 (s, 1H), 10.55 (s, 1H). | Intermediate 325 methyl 5-{2- chloro-4-[3- (trifluoro- methyl)-1H- pyrazol-1- yl]pyrimidin- 5-yl}-2- methoxy- pyridine-3- carboxylate |
| Example 735 | 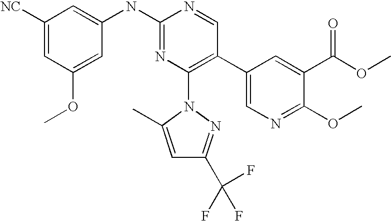 methyl 5-{2-[(3-cyano-5- methoxyphenyl)amino]-4-[5- methyl-3-(trifluoromethyl)- 1H-pyrazol-1-yl]pyrimidin- 5-yl}-2-methoxypyridine-3- carboxylate | MS(ES): 540 (M + 1) for C₂₅H₂₀F₃N₇O₄. 400 MHz, DMSO-d₆: δ 2.31 (s, 3H), 3.75 (s, 3H), 3.80 (s, 3H), 3.92 (s, 3H), 6.77 (s, 1H), 7.11 (s, 1H), 7.62 (d, J = 2.48 Hz, 1H), 7.71 (d, J = 2.00 Hz, 1H), 7.82 (s, 1H), 8.24 (d, J = 2.84 Hz, 1H), 9.01 (s, 1H), 10.57 (s, 1H). | Intermediate 273 methyl 5-{2- chloro-4-[3- methyl-3- (trifluoro- methyl)-1H- pyrazol-1- yl]pyrimidin- 5-yl}-2- methoxy- pyridine-3- carboxylate |

### Example 736

### 5-{2-[(3-cyano-5-methoxyphenyl)amino]-4-[3-(trifluoromethvl)-1H-pyrazol-1-yl]pyrimidin-5-yl}-2-methoxypyridine-3-carboxylic acid

### Example 737

### 5-{2-[(3-cyano-5-methoxyphenyl)amino]-4-[5-methyl-3-(trifluoromethyl)-1H-pyrazol-1-yl]pyrimidin-5-yl}-2-methoxypyridine-3-carboxylic acid

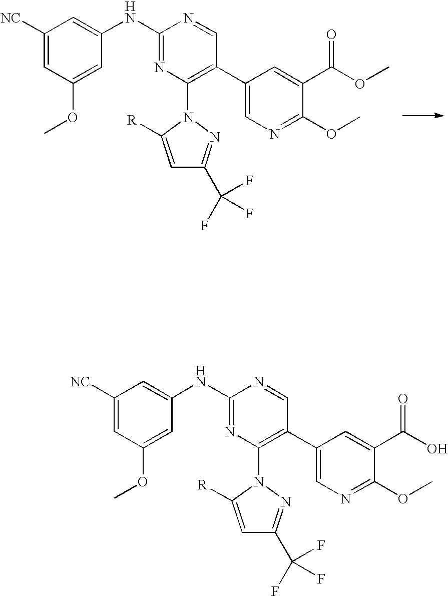

To methyl 5-{2-[(3-cyano-5-methoxyphenyl)amino]-4-[3-(trifluoromethyl)-1H-pyrazol-1-yl]pyrimidin-5-yl}-2-methoxypyridine-3-carboxylate (Example 734, 1 eq) or methyl 5-{2-[(3-cyano-5-methoxyphenyl)amino]-4-[5-methyl-3-(trifluoromethyl)-1H-pyrazol-1-yl]pyrimidin-5-yl}-2-methoxypyridine-3-carboxylate (Example 735, 1 eq) dissolved in a mixture of dioxane (5 mL) and water (5 mL), was added sodium hydroxide (2.5 eq) and stirred at room temperature for 3-5 h. The reaction mixture was carefully acidified with 1 N HCl and further extracted with ethyl acetate (50 mL). The organic layer was washed with water and brine, dried over Na₂SO₄ and concentrated. The compounds in the below table were prepared using this method and the specified starting material.

| | | Mass spectrum and | |
|---|---|---|---|
| Compound | Structure | ¹H NMR | SM |
| Example 736 | 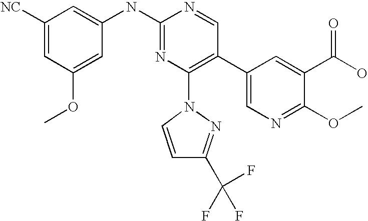 5-{2-[(3-cyano-5- methoxyphenyl)amino]-4-[3- (trifluoromethyl)-1H- pyrazol-1-yl]pyrimidin-5- yl}-2-methoxypyridine-3- carboxylic acid | MS(ES): 512 (M + 1) for C₂₃H₁₆F₃N₇O₄. 400 MHz, DMSO-d₆: δ 3.81 (s, 3H), 3.92 (s, 3H), 7.04 (d, J = 2.32 Hz, 1H), 7.09 (d, J = 1.08 Hz, 1H), 7.79 (d, J = 1.84 Hz, 2H), 7.81 (s, 1H), 8.22 (s, 1H), 8.47 (s, 1H), 8.83 (s, 1H), 10.54 (s, 1H), 12.95 (s, 1H). | Example 734 methyl 5-{2- [(3-cyano-5- methoxy- phenyl)amino]-4-[3- (trifluoro- methyl)-1H- pyrazol-1- yl]pyrimidin- 5-yl}-2- methoxy- pyridine-3- carboxylate |
| Example 737 | 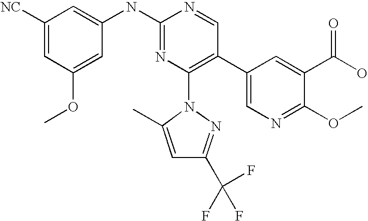 5-{2-[(3-cyano-5- methoxyphenyl)amino]-4-[5- methyl-3-(trifluoromethyl)- 1H-pyrazol-1-yl]pyrimidin- 5-yl}-2-methoxypyridine-3- carboxylic acid | MS(ES): 526 (M + 1) for C₂₄H₁₈F₃N₇O₄. 400 MHz, DMSO-d₆: δ 2.33 (s, 3H), 3.81 (s, 3H), 3.91 (s, 3H), 6.77 (s, 1H), 7.11 (s, 1H), 7.63 (d, J = 2.40 Hz, 1H), 7.72 (t, J = 1.92 Hz, 1H), 7.83 (s, 1H), 8.14 (s, 1H), 9.00 (s, 1H), 10.57 (s, 1H), 13.02 (br s, 1H). | Example 735 methyl 5-{2- [(3-cyano-5- methoxyphenyl)- amino]-4-[5- methyl-3- (trifluoro- methyl)-1H- pyrazol-1- yl]pyrimidin- 5-yl}-2- methoxy- pyridine-3- carboxylate |

### Example 738

### methyl 5-{2-[(3-chlorophenyl)amino]-4-[3-(trifluoromethyl)-1H-pyrazol-1-yl]pyrimidin-5-yl}-2-methoxypyridine-3-carboxylate

### Example 739

### methyl 5-{2-[(3-chlorophenyl)amino]-4-[5-methyl-3-(trifluoromethyl)-1H-pyrazol-1-yl]pyrimidin-5-yl}-2-methoxypyridine-3-carboxylate

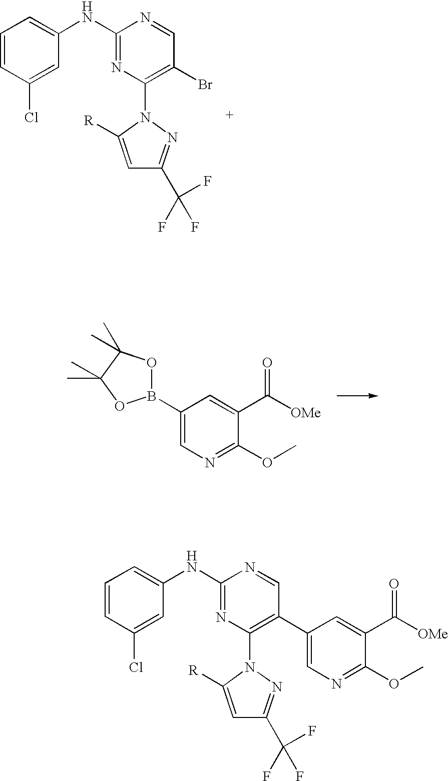

A suspension of 5-bromo-N-(3-chlorophenyl)-4-[3-(trifluoromethyl)-1H-pyrazol-1-yl]pyrimidin-2-amine Intermediate 276 or 5-bromo-N-(3-chlorophenyl)-4-[5-methyl-3-(trifluoromethyl)-1H-pyrazol-1-yl]pyrimidin-2-amine Intermediate 277 (1 eq), methyl 2-methoxy-5-(4,4,5,5-tetramethyl-1,3,2-dioxaborolan-2-yl)pyridine-3-carboxylate (1.1 eq), [1,1′-bis(diphenylphosphino)ferrocene]dichloropalladium(II) complex with CH₂Cl₂ (10 mol %) and sodium carbonate (1.1 eq) in acetonitrile/water (4:1) was degassed and heated to 100° C. for 45 minutes under an inert atmosphere. The reaction mass was passed through a celite bed. The solvent was concentrated in vacuo and the resultant crude mass was taken in CHCl₃ (50 mL), washed with water, brine, dried over Na₂SO₄, filtered and concentrated. The crude material was purified by silica gel column chromatography using 1:1 ethyl acetate/hexanes as an eluent to give the product. The compounds in the below table were prepared using this method and the specified starting material.

| | | Mass spectrum and ¹H | |
|---|---|---|---|
| Compound | Structure | NMR | SM |
| Example 738 | 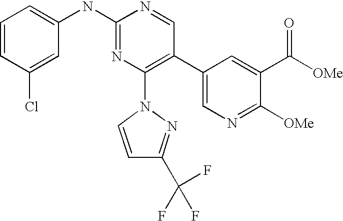 methyl 5-{2-[(3-chlorophenyl)amino]-4-[3- (trifluoromethyl)-1H-pyrazol-1-yl]pyrimidin-5-yl}- 2-methoxypyridine-3-carboxylate | MS (ES): 505 (M + 1) for C₂₂H₁₆ClF₃N₆O₃. 300 MHz, DMSO-d₆: δ 3.75 (s, 3H), 3.93 (s, 3H), 7.04 (m, 1H), 7.10 (s, 1H), 7.36 (t, J = 8.19 Hz, 1H), 7.70 (d, J = 6.75 Hz, 1H), 7.85 (d, J = 2.43 Hz, 1H), 7.96 (s, 1H), 8.27 (d, J = 2.40 Hz, 1H), 8.50 (s,1H), 8.80 (s, 1H), 10.41 (s, 1H). | Intermediate 276 5-bromo-N-(3- chlorophenyl)-4- [3-(trifluoromethyl)- 1H-pyrazol-1- yl]pyrimidin- 2-amine |
| Example 739 | 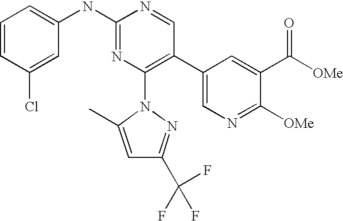 methyl 5-{2-[(3-chlorophenyl)amino]-4-[5-methyl-3- (trifluoromethyl)-1H-pyrazol-1-yl]pyrimidin-5-yl}-2- methoxypyridine-3-carboxylate | MS (ES): 519 (M + 1) for C₂₃H₁₈ClF₃N₆O₃. 300 MHz, DMSO-d₆: δ 2.33 (s, 3H), 3.73 (s, 3H), 3.96 (s, 3H), 6.76 (s, 1H), 7.07 (d, J = 8.01 Hz, 1H), 7.36 (t, J = 8.13 Hz, 1H), 7.60 (d, J = 2.46 Hz, 1H), 7.64 (d, J = 9.6 Hz, 1H), 7.97 (s, 1H), 8.22 (d, J = 2.10 Hz, 1H), 8.97 (s, 1H), 10.45 (s, 1H). | Intermediate 277 5-bromo-N-(3- chlorophenyl)-4- [5-methyl-3- (trifluoromethyl)- 1H-pyrazol-1- yl]pyrimidin- 2-amine |

### Example 740

### ethyl 5-{2-[(3-chlorophenyl)amino]-4-[3-(trifluoromethyl)-1H-pyrazol-1-yl]pyrimidin-5-yl}hyridine-3-carboxylate

### Example 741

### ethyl 5-{2-[(3-chlorophenyl)amino]-4-[5-methyl-3-(trifluoromethyl)-1H-pyrazol-1-yl]pyrimidin-5-yl}pyridine-3-carboxylate

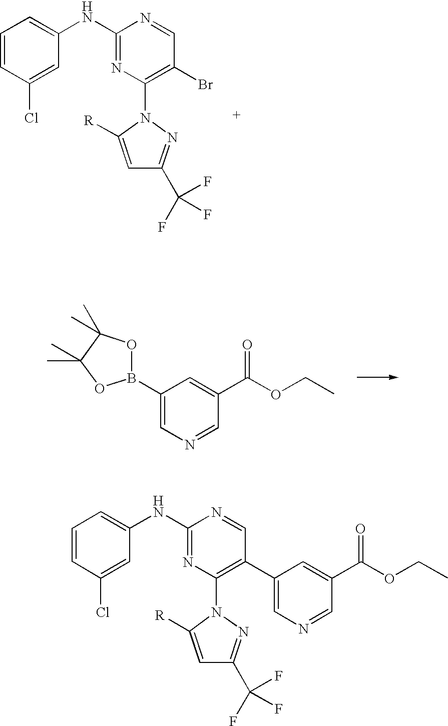

A suspension of 5-bromo-N-(3-chlorophenyl)-4-[3-(trifluoromethyl)-1H-pyrazol-1-yl]pyrimidin-2-amine or 5-bromo-N-(3-chlorophenyl)-4-[5-methyl-3-(trifluoromethyl)-1H-pyrazol-1-yl]pyrimidin-2-amine (1 eq), (1 eq), ethyl 5-(4,4,5,5-tetramethyl-1,3,2-dioxaborolan-2-yl)pyridine-3-carboxylate (1.1 eq), [1,1′-bis(diphenylphosphino)ferrocene]dichloropalladium(II)complex with CH₂Cl₂ (10 mol %) and sodium carbonate (1.1 eq) in acetonitrile/water (4:1) was degassed and heated to 100° C. for 45 min. The reaction mass was passed through a celite bed. The solvent was concentrated in vacuo and the resultant crude mass was taken in CHCl₃ (50 mL), washed with water, brine, dried over Na₂SO₄, filtered and concentrated. The crude material was purified by silica gel column chromatography using 1:1 ethyl acetate/hexanes as an eluent to give the product. The compounds in the below table were prepared using this method and the specified starting material.

| | | Mass spectrum and ¹H | |
|---|---|---|---|
| Compound | Structure | NMR | SM |
| Example 740 | 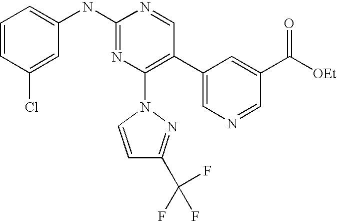 ethyl 5-{2-[(3-chlorophenyl)amino]-4-[3- (trifluoromethyl)-1H-pyrazol-1-yl]pyrimidin-5- yl}pyridine-3-carboxylate | MS (ES): 489 (M + 1) for C₂₂H₁₆ClF₃N₆O₂. 300 MHz, DMSO-d₆: δ 1.29 (m, 3H), 4.30 (q, J = 7.14 Hz, 2H), 7.03 (d, J = 1.32 Hz, 1H), 7.08 (d, J = 5.64 Hz, 1H), 7.38 (t, J = 8.10 Hz, 1H), 7.72 (d, J = 8.85 Hz, 1H), 7.97 (s, 1H), 8.03 (s, 1H), 8.55 (s, 1H), 8.69 (s, 1H), 8.85 (d, J = 2.22 Hz, 1H), 9.01 (s, 1H), 10.49 (s, 1H). | Intermediate 276 5-bromo-N-(3- chlorophenyl)-4- [3-(trifluoromethyl)- 1H-pyrazol-1- yl]pyrimidin- 2-amine |
| Example 741 | 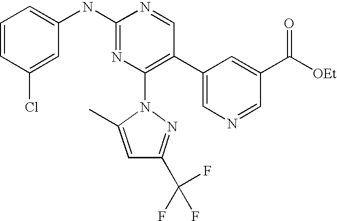 ethyl 5-{2-[(3-chlorophenyl)amino]-4-[5-methyl-3- (trifluoromethyl)-1H-pyrazol-1-yl]pyrimidin-5- yl}pyridine-3-carboxylate | MS (ES): 503 (M + 1) for C₂₃H₁₈ClF₃N₆O₂. 300 MHz, DMSO-d₆: δ 1.29 (t, J = 7.08 Hz, 3H), 2.43 (s, 3H), 4.30 (q, J = 7.08 Hz, 2H), 6.77 (s, 1H), 7.09 (d, J = 8.01 Hz, 1H), 7.37 (t, J = 8.13 Hz, 1H), 7.64 (d, J = 7.92 Hz, 1H), 7.80 (s, 1H), 7.98 (s, 1H), 8.60 (s, 1H), 8.97 (d, J = 1.83 Hz, 1H), 9.01 (s,1H), 10.50 (s, 1H). | Intermediate 277 5-bromo-N-(3- chlorophenyl)-4- [5-methyl-3- (trifluoro-methyl)- 1H-pyrazol-1- yl]pyrimidin- 2-amine |

### General Methods for Carboxylic Ester Hydrolysis

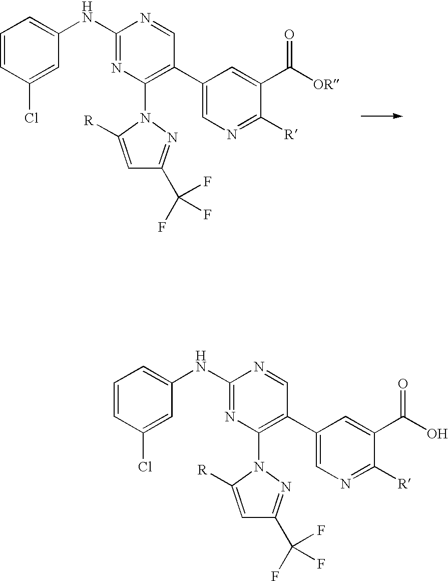

To a suspension of ester derivative (100 mg, 1 eq) taken in a mixture of dioxane (1 mL) and water (0.33 mL), was added Barium hydroxide (2 eq) and the mixture was allowed to stir at 45° C. for 2 h. The mixture was carefully acidified with 1 N HCl and the precipitate formed was filtered, washed with water and dried to yield the product. The compounds in the below table were prepared using this method and the specified starting material.

| | | Mass spectrum and ¹H | |
|---|---|---|---|
| Compound | Structure | NMR | SM |
| Example 742 | 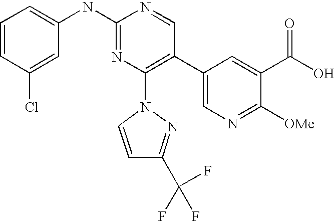 5-{2-[(3-chlorophenyl)amino]-4-[3-(trifluoromethyl)- 1H-pyrazol-1-yl]pyrimidin-5-yl}-2-methoxypyridine-3- carboxylic acid | MS (ES): 491 (M + 1) for C₂₁H₁₄ClF₃N₆O₃. 400 MHz, DMSO-d₆: δ 3.90 (s, 3H), 7.03 (d, J = 2.40 Hz, 1H), 7.06-7.08 (m, 1H), 7.37 (t, J = 8.20 Hz, 1H), 7.71 (d, J = 8.40 Hz, 1H), 7.82 (d, J = 2.40 Hz, 1H), 7.98 (s, 1H), 8.23 (d, J= 2.32 Hz, 1H), 8.49 (d, J = 1.04 Hz, 1H), 8.80 (s, 1H), 10.41 (s, 1H), 12.94 (s, 1H). | Example 738 methyl 5-{2-[(3- chlorophenyl)- amino]-4-[3- (trifluoromethyl)- 1H-pyrazol-1- yl]pyrimidin-5-yl}- 2-methoxy-pyridine- 3-carboxylate |
| Example 743 | 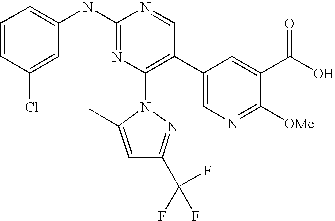 5-{2-[(3-chlorophenyl)amino]-4-[5-methyl-3- (trifluoromethyl)-1H-pyrazol-1-yl]pyrimidin-5-yl}-2- methoxypyridine-3-carboxylic acid | MS (ES): 505 (M + 1) for C₂₂H₁₆ClF₃N₆O₃. 400 MHz, DMSO-d₆: δ 2.35 (s, 3H), 3.90 (s, 3H), 6.76 (s, 1H), 7.08 (dd, J = 1.32, 7.96 Hz, 1H), 7.37 (t, J = 8.16 Hz, 1H), 7.62 (d, J = 2.48 Hz, 1H), 7.64-7.66 (m, 1H), 7.98 (s, 1H), 8.16 (d, J = 2.36 Hz, 1H), 8.96 (s, 1H), 10.45 (s, 1H), 12.98 (s,1H). | Example 739 methyl 5-{2-[(3- chlorophenyl)- amino]-4-[5-methyl- 3-(trifluoromethyl)- 1H-pyrazol-1- yl]pyrimidin-5-yl}- 2-methoxypyridine- 3-carboxylate |
| Example 744 | 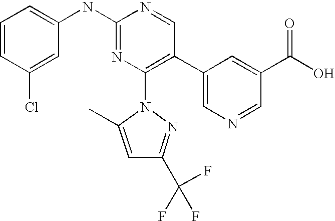 5-{2-[(3-chlorophenyl)amino]-4-[5-methyl-3- (trifluoromethyl)-1H-pyrazol-1-yl]pyrimidin-5- yl}pyridine-3-carboxylic acid | MS (ES): 475 (M + 1) for C₂₁H₁₄ClF₃N₆O₂. 400 MHz, DMSO-d₆: δ 2.44 (s, 3H), 6.77 (s, 1H), 7.09 (dd, J = 1.40, 7.94 Hz, 1H), 7.38 (t, J = 8.12 Hz, 1H), 7.65 (dd, J = 1.40, 8.16 Hz, 1H), 7.82 (t, J = 2.12 Hz, 1H), 7.98 (s, 1H), 8.54 (d, J = 2.24 Hz, 1H), 8.96 (d, J = 1.92 Hz, 1H), 9.00 (s, 1H), 10.50 (s, 1H), 13.46 (br s, 1H). | Example 741 ethyl 5-{2-[(3- chlorophenyl)- amino]-4-[5-methyl- 3-(trifluoro-methyl)- 1H-pyrazol-1- yl]pyrimidin-5- yl}pyridine-3- carboxylate |

Ester derivative (100 mg, leq) was dissolved in tetrahydrofuran (3 mL) and treated with potassium trimethyl silanolate (10 eq) and allowed to stir at room temperature for 1 h. The solvent was concentrated in vacuo and the resultant crude mass was carefully acidified with 1 N HCl, then diluted with ethyl acetate (50 mL), washed with water, brine, dried over Na₂SO₄, filtered and concentrated. The crude material was purified by silica gel column chromatography using chloroform:methanol (9:1) as an eluent to yield the product. The compound in the below table was prepared using this method and the specified starting material.

| | | Mass spectrum and ¹H | |
|---|---|---|---|
| Compound | Structure | NMR | SM |
| Example 745 | 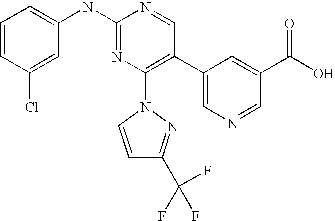 5-{2-[(3-chlorophenyl)amino]-4-[3-(trifluoromethyl)- 1H-pyrazol-1- yl]pyrimidin-5-yl}pyridine-3-carboxylic acid | MS (ES): 461 (M + 1) for C₂₀H₁₂ClF₃N₆O₂. 400 MHz, DMSO-d₆: δ 7.03 (d, J = 1.72 Hz, 1H), 7.08 (d, J = 7.52 Hz, 1H), 7.38 (t, J = 8.12 Hz, 1H), 7.73 (d, J = 8.44 Hz, 1H), 8.00 (d, J = 8.60 Hz, 2H), 8.51 (m, 2H), 8.82 (s, 1H), 8.99 (s, 1H), 10.46 (s, 1H), 12.89 (s, 1H). | Example 740 ethyl 5-{2-[(3- chlorophenyl)- amino]-4-[3- (trifluoromethyl)- 1H-pyrazol-1- yl]pyrimidin-5- yl}pyridine-3- carboxylate |

### General method for the synthesis of methyl 5-(2-{arylamino}-4-[1H-azol-1-yl]pyrimidin-5-yl)-2-methoxypyridine-3-carboxylate

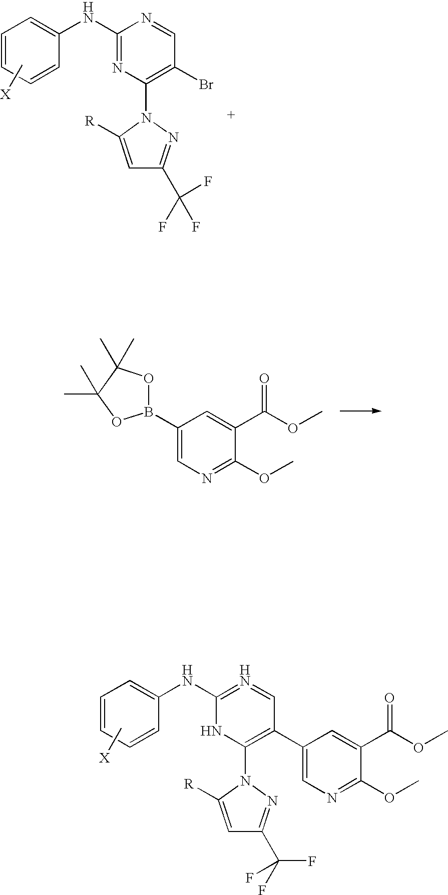

A suspension of 5-bromopyrimidine derivative (1 eq), methyl 2-methoxy-5-(4,4,5,5-tetramethyl-1,3,2-dioxaborolan-2-yl)pyridine-3-carboxylate (1.1-1.2 eq), [1,1′-bis(diphenylphosphino)ferrocene]-dichloropalladium(II) (10-20 mol %) and sodium carbonate (1-2 eq) in acetonitrile/water (4:1) was degassed and heated to 90° C. for 15-30 minutes under inert atmosphere. The solvent was removed in vacuo and the crude mixture taken in CHCl₃ was washed with water, brine, dried over Na₂SO₄, filtered and concentrated. The crude material was purified by silica gel column chromatography using 50% ethyl acetate/hexanes as eluent. The compounds in the below table were prepared using this procedure and the specified starting material.

| Compound | Structure | Mass spectrum and ¹H NMR | SM |
|---|---|---|---|
| Example 746 | 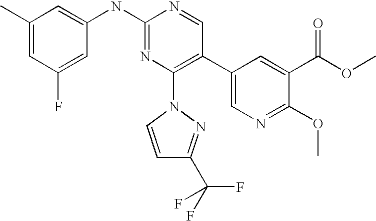 methyl 5-{2-[(3-fluoro-5-methylphenyl)amino]-4-[3- (trifluoromethyl)-1H-pyrazol-1-yl]pyrimidin-5-yl}-2- methoxypyridine-3-carboxylate | MS(ES): 503 (M + 1) for C₂₃H₁₈F₄N₆O₃. 400 MHz, DMSO-d₆: δ 2.32 (s, 3H), 3.77 (s, 3H), 3.95 (s, 3H), 6.70 (d, J = 9.48 Hz, 1H), 7.05 (d, J = 2.64 Hz, 1H), 7.37 (s, 1H), 7.60 (d, J = 11.80 Hz, 1H), 7.86 (d, J = 2.52 Hz, 1H), 8.29 (d, J = 2.48 Hz, 1H), 8.51 (t, J = 1.68 Hz, 1H), 8.80 (s, 1H), 10.37 (s, 1H). | Intermediate 287 5-bromo-N-(3- fluoro-5- methylphenyl)-4-[3- (trifluoromethyl)- 1H-pyrazol-1- yl]pyrimidin- 2-amine |
| Example 747 | 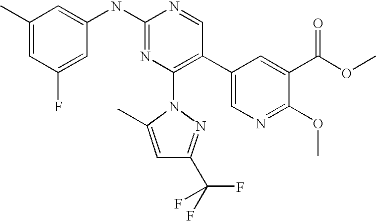 methyl 5-{2-[(3-fluoro-5-methylphenyl)amino]-4-[5- methyl-3-(trifluoromethyl)-1H-pyrazol-1-yl]pyrimidin- 5-yl}-2-methoxypyridine-3-carboxylate | MS(ES): 517 (M + 1) for C₂₄H₂₀F₄N₆O₃. 400 MHz, DMSO-d₆: δ 2.31 (s, 3H), 2.33 (s, 3H), 3.75 (s, 3H), 3.92 (s, 3H), 6.70 (d, J = 9.80 Hz, 1H), 6.77 (s, 1H), 7.33 (s, 1H), 7.58 (d, J = 11.60 Hz, 1H), 7.61 (d, J = 2.52 Hz, 1H), 8.24 (d, J = 2.48 Hz, 1H), 8.97 (s, 1H), 10.41 (s, 1H). | Intermediate 288 5-bromo-N-(3- fluoro-5- methylphenyl)-4-[5- methyl-3- (trifluoromethyl)- 1H-pyrazol-1- yl]pyrimidin- 2-amine |
| Example 748 | 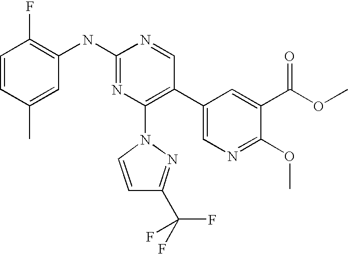 methyl 5-{2-[(2-fluoro-5-methylphenyl)amino]-4-[3- (trifluoromethyl)-1H-pyrazol-1-yl]pyrimidin-5-yl}- 2-methoxypyridine-3-carboxylate | MS(ES): 503 (M + 1) for C₂₃H₁₈F₄N₆O₃. 400 MHz, DMSO-d₆: δ 2.32 (s, 3H), 3.77 (s, 3H), 3.94 (s, 3H), 7.00 (d, J = 2.60 Hz, 1H), 7.03-7.05 (m, 1H), 7.18 (dd, J = 8.32, 10.68 Hz,1H), 7.51 (dd, J = 5.92, 6.48 Hz, 1H), 7.86 (d, J = 2.48 Hz, 1H), 8.27 (d, J = 2.48 Hz, 1H), 8.41 (s, 1H), 8.68 (s, 1H), 9.77 (s, 1H). | Intermediate 289 5-bromo-N-(2- fluoro-5- methylphenyl)-4-[3- (trifluoro-methyl)- 1H-pyrazol-1- yl]pyrimidin- 2-amine |
| Example 749 | 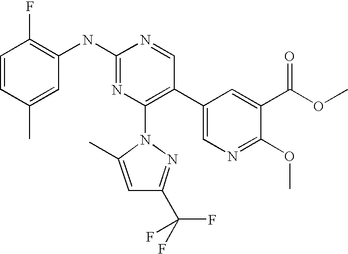 methyl 5-{2-[(2-fluoro-5-methylphenyl)amino]- 4-[5-methyl-3-(trifluoromethyl)-1H-pyrazol-1- yl]pyrimidin-5-yl}-2-methoxypyridine-3-carboxylate | Taken to the next step based on LCMS: MS(ES): 517 (M + 1) for C₂₄H₂₀F₄N₆O₃. | Intermediate 290 5-bromo-N-(2- fluoro-5- methylphenyl)-4-[5- methyl-3- (trifluoromethyl)- 1H-pyrazol-1- yl]pyrimidin- 2-amine |
| Example 750 | 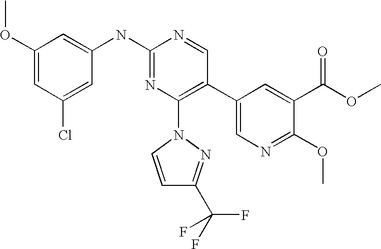 methyl 5-{2-[(3-chloro-5-methoxyphenyl)amino]- 4-[3-(trifluoromethyl)-1H-pyrazol-1-yl]pyrimidin-5- yl}-2-methoxypyridine-3-carboxylate | MS(ES): 535 (M + 1) for C₂₃H₁₈ClF₃N₆O₄. 300 MHz, DMSO-d₆: δ 3.76 (s, 3H), 3.77 (s, 3H), 3.93 (s, 3H), 6.68 (s, 1H), 7.04 (d, J = 2.55 Hz, 1H), 7.45 (s, 1H), 7.51 (s, 1H), 7.85 (d, J = 2.46 Hz, 1H), 8.27 (d, J = 2.46 Hz, 1H), 8.46 (s, 1H), 8.81 (s, 1H), 10.38 (s, 1H). | Intermediate 291 5-bromo-N-(3- chloro-5- methoxyphenyl)-4- [3-(trifluoromethyl)- 1H-pyrazol-1- yl]pyrimidin- 2-amine |
| Example 751 | 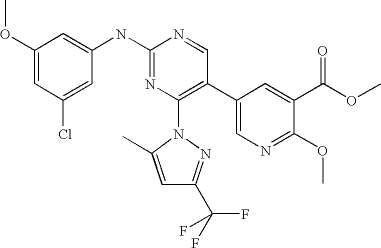 methyl 5-{2-[(3-chloro-5-methoxyphenyl)amino]- 4-[5-methyl-3-(trifluoromethyl)-1H-pyrazol-1- yl]pyrimidin-5-yl}-2-methoxypyridine-3-carboxylate | MS(ES): 549 (M + 1) for C₂₄H₂₀ClF₃N₆O₄. 400 MHz, DMSO-d₆: δ 3.32 (s, 3H), 3.74 (s, 3H), 3.75 (s, 3H), 3.91 (s, 3H), 6.70 (s, 1H), 6.76 (s, 1H), 7.37 (s, 1H), 7.52 (s, 1H), 7.61 (d, J = 2.32 Hz, 1H), 8.23 (d, J = 2.36 Hz, 1H), 8.98 (s, 1H), 10.42 (s, 1H). | Intermediate 292 5-bromo-N-(3- chloro-5- methoxyphenyl)-4- [5-methyl-3- (trifluoromethyl)- 1H-pyrazol-1- yl]pyrimidin- 2-amine |
| Example 752 | 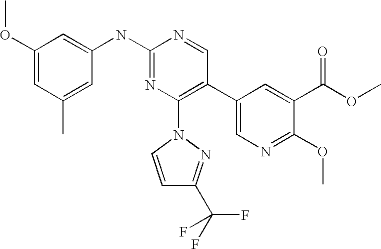 methyl 2-methoxy-5-{2-[(3-methoxy-5-methylphenyl)amino]- 4-[3-(trifluoromethyl)-1H-pyrazol-1-yl]pyrimidin-5- yl}pyridine-3-carboxylate | MS(ES): 515 (M + 1) for C₂₄H₂₁F₃N₆O₄. 400 MHz, DMSO-d₆: δ 2.28 (s, 3H), 3.74 (s, 3H), 3.77 (s, 3H), 3.95 (s, 3H), 6.46 (s, 1H), 7.04 (d, J = 2.64 Hz, 1H), 7.16 (s, 1H), 7.36 (s, 1H), 7.86 (d, J = 2.36 Hz, 1H), 8.28 (d, J = 2.40 Hz, 1H), 8.49 (d, J = 1.88 Hz, 1H), 8.77 (s, 1H), 10.15 (s, 1H). | Intermediate 293 5-bromo-N-(3- methoxy-5- methylphenyl)-4-[3- (trifluoromethyl)- 1H-pyrazol-1- yl]pyrimidin- 2-amine |
| Example 753 | 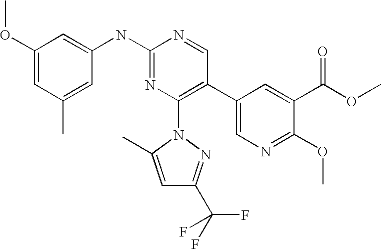 methyl 2-methoxy-5-{2-[(3-methoxy-5-methylphenyl)amino]- 4-[5-methyl-3-(trifluoromethyl)-1H-pyrazol-1-yl]pyrimidin- 5-yl}pyridine-3-carboxylate | MS(ES): 529 (M + 1) for C₂₅H₂₃F₃N₆O₄. 300 MHz, DMSO-d₆: δ 2.25 (s, 3H), 2.31 (s, 3H), 3.70 (s, 3H), 3.74 (s, 3H), 3.90 (s, 3H), 6.45 (s, 1H), 6.74 (s, 1H), 7.11 (s, 1H), 7.27 (s, 1H), 7.59 (d, J = 2.49 Hz, 1H), 8.21 (d, J = 2.52 Hz, 1H), 8.91 (s, 1H), 10.15 (s, 1H). | Intermediate 294 5-bromo-N-(3- methoxy-5- methylphenyl)-4-[5- methyl-3- (trifluoromethyl)- 1H-pyrazol-1- yl]pyrimidin- 2-amine |

### General procedure for the synthesis of 5-{2-(arylamino)-4-[1H-pyrazol-1-yl]pyrimidin-5-yl}-2-methoxypyridine-3-carboxylic acid

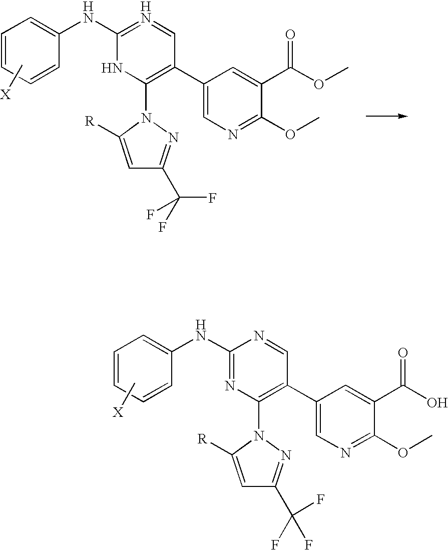

To 1 eq of methyl 5-(2-{arylamino}-4-[1H-azol-1-yl]pyrimidin-5-yl)-2-methoxypyridine-3-carboxylate taken in dioxane (5 mL), was added 1 N aq. Barium hydroxide (2 eq) and stirred for the amount of time indicated in the below table. The reaction mixture was carefully acidified with 1 N HCl and the solid that precipitated was filtered, washed with water and dried in vacuo to yield the carboxylic acid product. The compounds in the below table were prepared using this procedure and the specified starting material.

| Compound | Structure | Mass spectrum and ¹H NMR | SM |
|---|---|---|---|
| Example 754^{b)} | 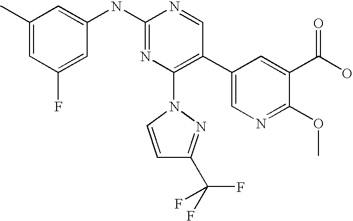 5-{2-[(3-fluoro-5-methylphenyl)-amino]-4-[3- (trifluoromethyl)-1H-pyrazol-1-yl]pyrimidin-5-yl}- 2-methoxypyridine-3-carboxylic acid | MS(ES): 489 (M + 1) for C₂₂H₁₆F₄N₆O₃. 400 MHz, DMSO-d₆: δ 2.32 (s, 3H), 3.93 (s, 3H), 6.70 (d, J = 8.96 Hz, 1H), 7.04 (d, J = 2.52 Hz, 1H), 7.38 (s, 1H), 7.61 (d, J = 11.72 Hz, 1), 7.82 (d, J = 2.36 Hz, 1H), 8.24 (d, J = 2.20 Hz, 1H), 8.49 (s, 1H), 8.80 (s, 1H), 10.37 (s, 1H), 12.96 (s, 1H). | Example 746 methyl 5-{2-[(3- fluoro-5- methylphenyl)- amino]-4-[3- (trifluoromethyl)- 1H-pyrazol-1- yl]pyrimidin-5- yl}-2- methoxypyridine- 3-carboxylate |
| Example 755^{b)} | 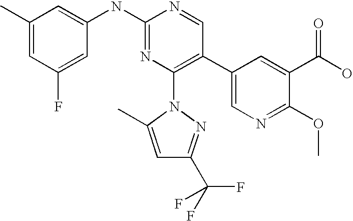 5-{2-[(3-fluoro-5-methylphenyl)amino]-4-[5-methyl- 3-(trifluoromethyl)-1H-pyrazol-1-yl]pyrimidin-5-yl}- 2-methoxypyridine-3-carboxylic acid | MS(ES): 503 (M + 1) for C₂₃H₁₈F₄N₆O₃. 400 MHz, DMSO-d₆: δ 2.30 (s, 3H), 2.34 (s, 3H), 3.90 (s, 3H), 6.69 (d, J = 9.52 Hz, 1H), 6.75 (s, 1H), 7.32 (s, 1H), 7.57 (d, J = 11.76 Hz, 1H), 7.62 (d, J = 2.52 Hz, 1H), 8.16 (d, J = 2.52 Hz, 1H), 8.95 (s, 1H), 10.38 (s, 1H), 12.95 (s, 1H). | Example 747 methyl 5-{2-[(3- fluoro-5- methylphenyl)- amino-]-4-[5- methyl-3- (trifluoromethyl)- 1H-pyrazol-1- yl]pyrimidin- 5-yl}-2- methoxypyridine- 3-carboxylate |
| Example 756^{c)} | 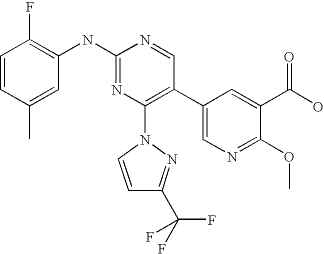 5-{2-[(2-fluoro-5-methylphenyl)amino]-4-[3- (trifluoromethyl)-1H-pyrazol-1-yl]pyrimidin-5-yl}- 2-methoxypyridine-3-carboxylic acid | MS(ES): 489 (M + 1) for C₂₂H₁₆F₄N₆O₃. 400 MHz, DMSO-d₆: δ 2.31 (s, 3H), 3.93 (s, 3H), 7.01 (d, 1H), 7.03-7.04 (m, 1H), 7.17 (dd, J = 8.4, 10.32 Hz, 1H), 7.50 (dd, J = 1.72, 7.56 Hz, 1H), 7.83 (d, J = 2.48 Hz, 1H), 8.23(d, J = 2.52 Hz, 1H), 8.40 (s, 1H), 8.68 (s,1H), 9.75 (s, 1H), 12.93 (br s, 1H). | Example 748 methyl 5-{2-[(2- fluoro-5- methylphenyl)- amino]-4-[3- (trifluoromethyl)- 1H-pyrazol-1- yl]pyrimidin- 5-yl}-2- methoxypyridine- 3-carboxylate |
| Example 757^{c, e)} | 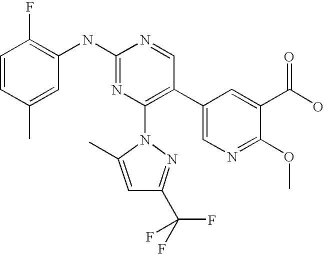 5-{2-[(2-fluoro-5-methylphenyl)amino]-4-[5-methyl- 3-(trifluoromethyl)-1H-pyrazol-01-yl]pyrimidin-5-yl}- 2-methoxypyridine-3-carboxylic acid | MS(ES): 503 (M + 1) for C₂₃H₁₈F₄N₆O₃. 400 MHz, DMSO-d₆: δ 2.28 (s, 3H), 2.29 (s, 3H), 3.88 (s, 3H), 6.69 (s, 1H), 7.02 (m, 1H), 7.15 (t, J = 10.44 Hz, 1H), 7.41 (d, J = 6.92 Hz, 1H), 7.60 (d, J = 2.44 Hz, 1H), 8.13 (d, J = 2.40 Hz, 1H), 8.81 (s, 1H), 9.78 (s, 1H), 12.92 (s, 1H). | Example 749 methyl 5-{2-[(2- fluoro-5- methylphenyl)- amino]-4-[5- methyl-3- (trifluoromethyl)- 1H-pyrazol-1- yl]pyrimidin- 5-yl}-2- methoxypyridine- 3-carboxylate |
| Example 758^{d)} | 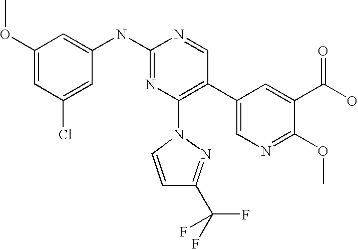 5-{2-[(3-chloro-5-methoxyphenyl)amino]-4-[3- (trifluoromethyl)-1H-pyrazol-1-yl]pyrimidin-5-yl}- 2-methoxypyridine-3-carboxylic acid | MS(ES): 521 (M + 1) for C₂₂H₁₆ClF₃N₆O₄. 400 MHz, DMSO-d₆: δ 3.79 (s, 3H), 3.94 (s, 3H), 6.70 (t, J = 1.96 Hz, 1H), 7.05 (d, J = 2.60 Hz, 1H), 7.48 (d, J = 1.92 Hz, 1H), 7.53 (s, 1H), 7.84 (d, J = 2.36 Hz, 1H), 8.25 (d, J = 2.36 Hz, 1), 8.47 (d, J = 1.64 Hz, 1H), 8.82 (s, 1H), 10.39 (s, 1H), 12.95 (s, 1H). | Example 750 methyl 5-{2-[(3- chloro-5- methoxyphenyl)- amino]-4-[3- (trifluoromethyl)- 1H-pyrazol-1- yl]pyrimidin- 5-yl}-2- methoxypyridine- 3-carboxylate |
| Example 759^{d)} | 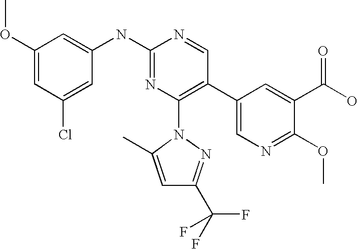 5-{2-[(3-chloro-5-methoxyphenyl)amino]-4-[5-methyl- 3-(trifluoromethyl)-1H-pyrazol-1-yl]pyrimidin-5-yl}- 2-methoxypyridine-3-carboxylic acid | MS(ES): 535 (M + 1) for C₂₃H₁₈ClF₃N₆O₄. 400 MHz, DMSO-d₆: δ 2.34 (s, 3H), 3.76 (s, 3H), 3.90 (s, 3H), 6.70 (s, 1H), 6.76 (s, 1H), 7.38 (s, 1H), 7.53 (s, 1H), 7.62 (d, J = 2.48 Hz, 1H), 8.17 (d, J = 2.48 Hz, 1H), 8.97 (s, 1H), 10.41 (s, 1H), 12.97 (s, 1H). | Example 751 methyl 5-{2-[(3- chloro-5- methoxyphenyl)- amino]-4-[5- methyl-3- (trifluoromethyl)- 1H-pyrazol-1- yl]pyrimidin- 5-yl}-2- methoxypyridine- 3-carboxylate |
| Example 760^{c)} | 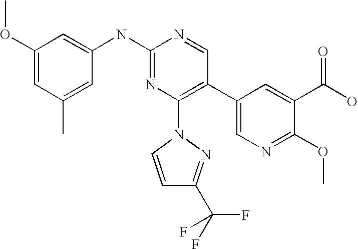 2-methoxy-5-{2-[(3-methoxy-5-methylphenyl)amino]-4- [3-(trifluoromethyl)-1-pyrazol-1-yl]pyrimidin-5- yl}pyridine-3-carboxylic acid | MS(ES): 501 (M + 1) for C₂₃H₁₉F₃N₆O₄. 400 MHz, DMSO-d₆: δ 2.28 (s, 3H), 3.74 (s, 3H), 3.92 (s, 3H), 6.45 (s, 1H), 7.02 (d, J = 2.60 Hz, 1H), 7.15 (s, 1H), 7.36 (s, 1H), 7.80 (d, J = 2.48 Hz, 1H), 8.20 (d, J = 2.44 Hz, 1H), 8.46 (d, J = 1.68 Hz, 1H), 8.75 (s, 1H), 10.12 (s, 1H), 12.98 (s, 1H). | Example 752 methyl 2- methoxy-5-{2- [(3-methoxy-5- methylphenyl)- amino]-4-[3- (trifluoromethyl)-1H-pyrazol-1- yl]pyrimidin-5- yl}pyridine-3- carboxylate |
| Example 761^{c)} | 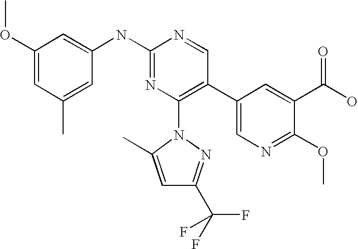 2-methoxy-5-{2-[(3-methoxy-5-methylphenyl)amino]- 4-[5-methyl-3-(trifluoromethyl)-1H-pyrazol-1- yl]pyrimidin-5-yl}pyridine-3-carboxylic acid | MS(ES): 515 (M + 1) for C₂₄H₂₁F₃N₆O₄. 400 MHz, DMSO-d₆: δ 2.26 (s, 3H), 2.32 (s, 3H), 3.71 (s, 3H), 3.89 (s, 3H), 6.45 (s, 1H), 6.74 (s, 1H), 7.12 (s, 1H), 7.28 (s, 1H), 7.61 (d, J = 2.40 Hz, 1H), 8.15 (d, J = 2.36 Hz, 1H), 8.90 (s, 1H), 10.15 (s, 1H), 12.96 (s, 1H). | Example 753 methyl 2- methoxy-5-{2- [(3-methoxy-5- methylphenyl)- amino]-4-[5- methyl-3- (trifluoromethyl)- 1H-pyrazol-1- yl]pyrimidin-5- yl}pyridine- 3-carboxylate |

^{b)}45° C., 2 h,
^{c)}40° C., 2 h,
^{d)}50° C., 45 min
^{e)}Purified by preparative HPLC

### General procedure for the synthesis of ethyl 5-(2-{arylamino}-4-[1H-azol-1-yl]pyrimidin-5-yl)pyridine-3-carboxylate

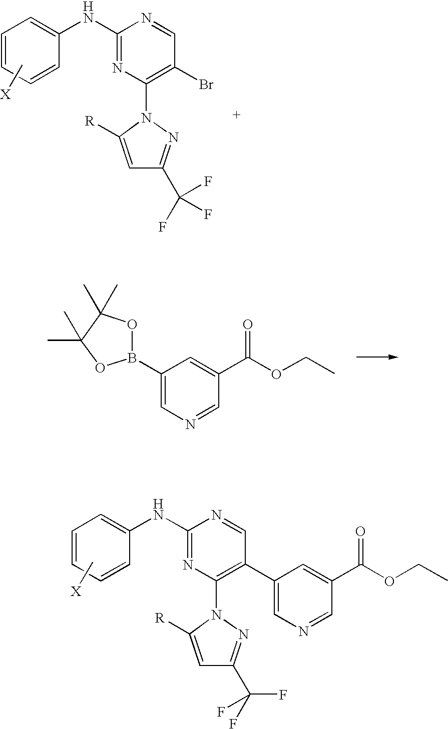

A suspension of 5-bromo-N-(aryl)-4-[3-(trifluoromethyl)-1H-pyrazol-1-yl]pyrimidin-2-amine or 5-bromo-N-(aryl)-4-[5-methyl-3-(trifluoromethyl)-1H-pyrazol-1-yl]pyrimidin-2-amine (1 eq), ethyl 5-(4,4,5,5-tetramethyl-1,3,2-dioxaborolan-2-yl)pyridine-3-carboxylate (1.1-1.2 eq), [1,1′-bis(diphenylphosphino)ferrocene]-dichloropalladium(II) (10-20 mol %) and sodium carbonate (1-2 eq) in acetonitrile/water (4:1) was degassed and heated to 90° C. for 15-30 min under inert atmosphere. The solvent was removed in vacuo and the crude mixture taken in CHCl₃ was washed with water, brine, dried over Na₂SO₄, filtered and concentrated. The crude material was purified by silica gel column chromatography using 30% ethyl acetate/hexanes as eluent to give the product. The compounds in the below table were prepared using this procedure and the specified starting material.

| Compound | Structure | Mass spectrum and ¹H NMR | SM |
|---|---|---|---|
| Example 762 | 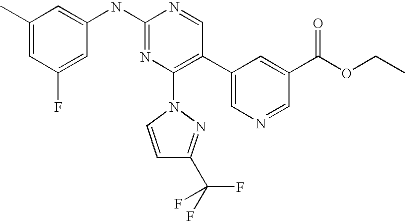 ethyl 5-{2-[(3-fluoro-5-methylphenyl)amino]-4-[3-(trifluoromethyl)- 1H-pyrazol-1-yl]pyrimidin-5-yl}pyridine-3-carboxylate | MS(ES): 487 (M + 1) for C₂₃H₁₈F₄N₆O₂. 300 MHz, DMSO-d₆: δ 1.29 (t, J = 7.05 Hz, 3H), 2.31 (s, 3H), 4.32 (q, J = 7.02 Hz, 2H), 6.70 (d, J = 9.81 Hz, 1H), 7.05 (s, 1H), 7.36 (s, 1H), 7.59 (d, J = 11.94 Hz, 1H), 8.03 (s, 1H), 8.54 (s, 1H), 8.70 (s, 1H), 8.84 (s, 1H), 9.00 (s, 1H), 10.42 (s, 1H). | Intermediate 287 5-bromo-N-(3- fluoro-5- methylphenyl)-4-[3- (trifluoromethyl)- 1H-pyrazol-1- yl]pyrimidin- 2-amine |
| Example 763 | 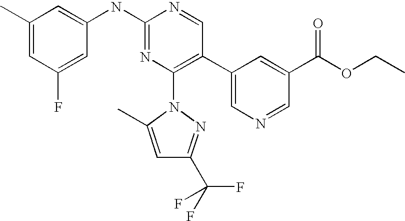 ethyl 5-{2-[(3-fluoro-5-methylphenyl)amino]-4-[5-methyl-3- (trifluoromethyl)-1H-pyrazol-1-yl]pyrimidin-5-yl}pyridine-3- carboxylate | MS(ES): 501 (M + 1) for C₂₄H₂₀F₄N₆O₂. 400 MHz, DMSO-d₆: δ 1.31 (t, J = 7.04 Hz, 3H), 2.31 (s, 3H), 2.43 (s, 3H), 4.32 (q, J = 7.08 Hz, 2H), 6.72 (d, J = 9.24 Hz, 1H), 6.79 (s, 1H), 7.34 (s, 1H), 7.58 (d, J = 11.72 Hz, 1H), 7.81 (t, J = 2.08 Hz, 1H), 8.62 (d, J = 2.20 Hz, 1H), 8.99 (d, J = 1.92 Hz, 1H), 9.00 (s, 1H), 10.46 (s, 1H). | Intermediate 288 5-bromo-N-(3- fluoro-5- methylphenyl)-4-[5- methyl-3- (trifluoromethyl)- 1H-pyrazol-1- yl]pyrimidin- 2-amine |
| Example 764 | 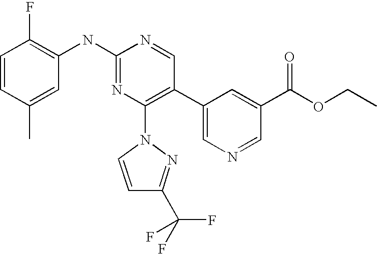 ethyl 5-{2-[(2-fluoro-5-methylphenyl)amino]-4-[3- (trifluoromethyl)-1H-pyrazol-1-yl]pyrimidin-5- yl}pyridine-3-carboxylate | MS(ES): 487 (M + 1) for C₂₃H₁₈F₄N₆O₂. 400 MHz, DMSO-d₆: δ 1.29 (t, J = 7.08 Hz, 3H), 2.31 (s, 3H), 4.32 (q, J = 7.16 Hz, 2H), 6.99 (d, J = 2.64 Hz, 1H), 7.03-7.07 (m, 1H), 7.17 (dd, J = 8.52, 10.66 Hz, 1H), 7.49 (d, J = 6.40 Hz, 1H), 8.02 (t, J = 2.04 Hz, 1H), 8.43 (s, 1H), 8.67 (d, J = 2.16 Hz, 1H), 8.71 (s, 1H), 9.01 (d, J = 1.96 Hz, 1H), 9.82 (s, 1H). | Intermediate 289 5-bromo-N-(2- fluoro-5- methylphenyl)-4-[3- (trifluoromethyl)- 1H-pyrazol-1- yl]pyrimidin- 2-amine |
| Example 765 | 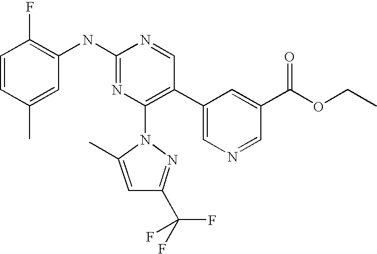 ethyl 5-{2-[(2-fluoro-5-methylphenyl)amino]-4-[5-methyl-3- (trifluoromethyl)-1H-pyrazol-1-yl]pyrimidin-5-yl}pyridine-3- carboxylate | MS(ES): 501 (M + 1) C₂₄H₂₀F₄N₆O₂. Taken to the next step based on LCMS without further purification. | Intermediate 290 5-bromo-N-(2- fluoro-5- methylphenyl)-4-[5- methyl-3- (trifluoromethyl)- 1H-pyrazol-1- yl]pyrimidin- 2-amine |
| Example 766 | 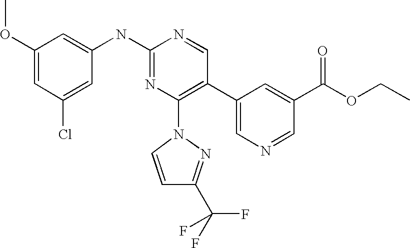 ethyl 5-{2-[(3-chloro-5-methoxyphenyl)amino]-4-[3- (trifluoromethyl)-1H-pyrazol-1-yl]pyrimidin-5- yl}pyridine-3-carboxylate | MS(ES): 519 (M + 1) for C₂₃H₁₈ClF₃N₆O₃ 300 MHz, DMSO-d₆: δ 1.29 (t, J = 7.08 Hz, 3H), 3.77 (s, 3H), 4.32 (q, J = 7.08 Hz, 2H), 6.70 (d, J = 1.98 Hz, 1H), 7.05 (d, J = 2.55 Hz, 1H), 7.46 (s, 1H), 7.52 (s, 1H), 8.03 (t, J = 2.07 Hz, 1H), 8.51 (s, 1H), 8.68 (d, J = 2.19 Hz, 1H), 8.86 (s, 1H), 9.02 (d, J = 1.98 Hz, 1H), 10.44 (s, 1H). | Intermediate 291 5-bromo-N-(3- chloro-5- methoxyphenyl)- 4-[3- (trifluoromethyl)- 1H-pyrazol-1- yl]pyrimidin- 2-amine |
| Example 767 | 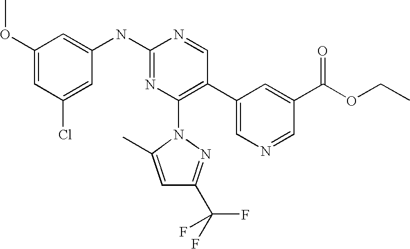 ethyl 5-{2-[(3-chloro-5-methoxyphenyl)amino]- 4-[5-methyl-3-(trifluoromethyl)-1H-pyrazol-1- yl]pyrimidin-5-yl}pyridine-3-carboxylate | MS(ES): 533 (M + 1) for C₂₄H₂₀ClF₃N₆O₃ 400 MHz, DMSO-d₆: δ 1.31 (t, J = 7.08 Hz, 3H), 2.43 (s, 3H), 3.77 (s, 3H), 4.32 (q, J = 7.08 Hz, 2H), 6.73 (d, J = 1.68 Hz, 1H), 6.80 (s, 1H), 7.38 (s, 1H), 7.55 (s,1H), 7.82 (t, J = 1.88 Hz, 1H), 8.62 (d, J = 2.00 Hz, 1H), 8.99 (d, J = 1.76 Hz, 1H), 9.04 (s, 1H), 10.48 (s, 1H). | Intermediate 292 5-bromo-N-(3- chloro-5- methoxyphenyl)-4- [5-methyl-3- (trifluoromethyl)- 1H-pyrazol-1- yl]pyrimidin- 2-amine |
| Example 768 | 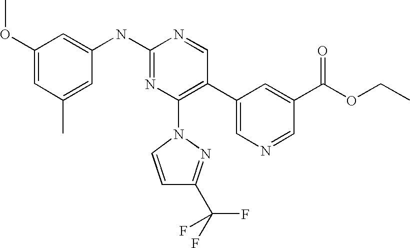 ethyl 5-{2-[(3-methoxy-5-methylphenyl)amino]- 4-[3-(trifluoromethyl)-1H-pyrazol-1-yl]pyrimidin-5- yl}pyridin-3-carboxylate | MS(ES): 499 (M + 1) for C₂₄H₂₁F₃N₆O₃. 400 MHz, DMSO-d₆: δ 1.31 (t, J = 7.04 Hz, 3H), 2.29 (s, 3H), 3.75 (s, 3H), 4.35 (q, J = 7.08 Hz, 2H), 6.48 (s, 1H), 7.06 (d, J = 2.52 Hz, 1H), 7.17 (s, 1H), 7.36 (s, 1H), 8.04 (t, J = 2.08 Hz, 1H), 8.54 (s,1H), 8.69 (d, J = 2.20 Hz, 1H), 8.82 (s, 1H), 9.03 (d, J = 1.92 Hz, 1H), 10.21 (s, 1H). | Intermediate 293 5-bromo-N-(3- methoxy-5- methylphenyl)-4- [3-(trifluoromethyl)- 1H-pyrazol-1- yl]pyrimidin- 2-amine |
| Example 769 | 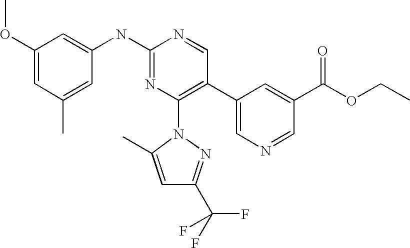 ethyl 5-{2-[(3-methoxy-5-methylphenyl)amino]-4- [5-methyl-3-(trifluoromethyl)-1H-pyrazol-1-yl]pyrimidin- 5-yl}pyridine-3-carboxylate | MS(ES): 513 (M + 1) for C₂₅H₂₃F₃N₆O₃. 400 MHz, DMSO-d₆: δ 1.31 (t, J = 7.08 Hz, 3H), 2.28 (s, 3H), 2.42 (s, 3H), 3.73 (s, 3H), 4.32 (q, J = 7.12 Hz, 2H), 6.49 (s, 1H), 6.78 (s, 1H), 7.15 (s, 1H), 7.29 (s, 1H), 7.80 (t, J = 2.12 Hz, 1H), 8.61 (d, J = 2.20 Hz, 1H), 8.98 (m, 2H), 10.23 (s, 1H). | Intermediate 294 5-bromo-N-(3- methoxy-5- methylphenyl)-4- [5-methyl-3- (trifluoromethyl)- 1H-pyrazol-1- yl]pyrimidin- 2-amine |

### General procedure for the synthesis of 5-{2-(arylamino)-4-[1H-pyrazol-1-yl]pyrimidin-5-yl}-2-methoxypyridine-3-carboxylic acid

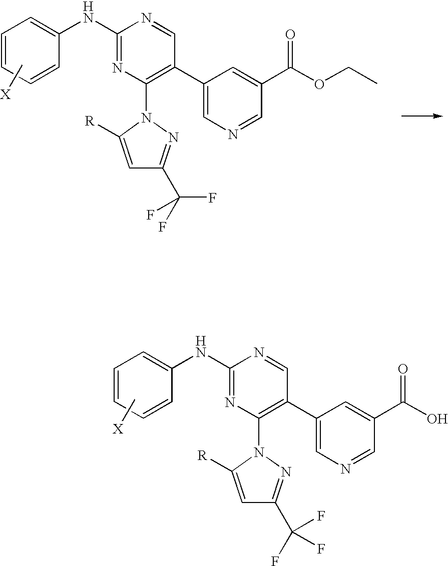

To 1 eq of ethyl 5-(2-{arylamino}-4-[1H-azol-1-yl]pyrimidin-5-yl)pyridine-3-carboxylate taken in dioxane (5 mL), was added 1 N aq. Barium hydroxide (2 eq) and stirred for the time indicated in the below table. The reaction mixture was carefully acidified with 1 N HCl and the solid that precipitated was filtered, washed with water and dried in vacuo to yield the carboxylic acid product. The compounds in the below table were prepared using this procedure and the specified starting material.

| Compound | Structure | Mass spectrum and ¹H NMR | SM |
|---|---|---|---|
| Example 770^{f)} | 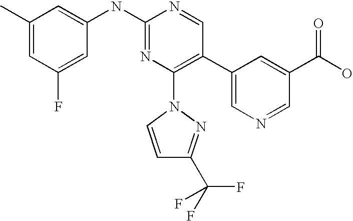 5-{2-[(3-fluoro-5-methylphenyl)-amino]-4-[3- (trifluoromethyl)-1H-pyrazol-1-yl]pyrimidin-5- yl}pyridine-3-carboxylic acid | MS(ES): 459 (M + 1) for C₂₁H₁₄F₄N₆O₂. 400 MHz, DMSO-d₆: δ 2.32 (s, 3H), 6.70 (d, J = 9.52 Hz, 1H), 7.05 (d, J = 2.64 Hz, 1H), 7.38 (s, 1H), 7.60 (d, J = 11.72 Hz, 1H), 8.02 (t, J = 2.08 Hz, 1H), 8.54 (d, J = 1.64 Hz, 1H), 8.65 (d, J = 2.20 Hz, 1H), 8.84 (s, 1H), 9.01 (d, J = 1.96 Hz, 1H), 10.41 (s, 1H), 13.42 (br s, 1H). | Example 762 ethyl 5-{2-[(3- fluoro-5- methylphenyl)- amino]-4-[3- (trifluoromethyl)- 1H-pyrazol-1- yl]pyrimidin-5- yl}pyridine-3- carboxylate |
| Example 771^{g)} | 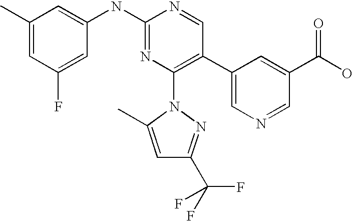 5-{2-[(3-fluoro-5-methylphenyl)-amino]-4-[5- methyl-3-(trifluoromethyl)-1H-pyrazol-1- yl]pyrimidin-5-yl}pyridine-3-carboxylic acid | MS(ES): 473 (M + 1) for C₂₂H₁₆F₄N₆O₂. 400 MHz, DMSO-d₆: δ 2.31 (s, 3H), 2.43 (s, 3H), 6.71 (d, J = 9.28 Hz, 1H), 6.77 (s, 1H), 7.33 (s, 1H), 7.57 (d, J = 11.56 Hz, 1H), 7.83 (t, J = 2.00 Hz, 1H), 8.54 (d, J = 2.16 Hz, 1H), 8.96 (d, J = 1.88 Hz, 1H), 9.00 (s, 1H), 10.43 (s, 1H), 13.43 (s, 1H). | Example 763 ethyl 5-{2-[(3- fluoro-5- methylphenyl)- amino]-4-[5- methyl-3- (trifluoromethyl)- 1H-pyrazol-1- yl]pyrimidin-5- yl}pyridine-3- carboxylate |
| Example 772^{h)} | 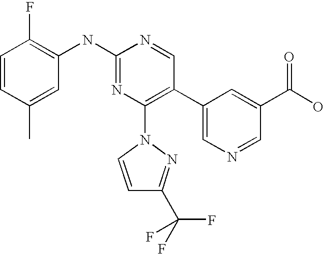 5-{2-[(2-fluoro-5-methylphenyl)-amino]-4-[3- (trifluoromethyl)-1H-pyrazol-1-yl]pyrimidin-5- yl}pyridine-3-carboxylic acid | MS(ES): 459 (M + 1) for C₂₁H₁₄F₄N₆O₂. 400 MHz, DMSO-d₆: δ 2.32 (s, 3H), 7.00 (d, J = 2.68 Hz, 1H), 7.02-7.06 (m, 1H), 7.19 (dd, J = 8.36, 10.68 Hz, 1H), 7.51 (dd, J = 1.80, 7.68 Hz, 1H), 8.02 (t, J = 2.08 Hz, 1H), 8.41 (s, 1H), 8.64 (d, J = 2.24 Hz, 1H), 8.72 (s, 1H), 9.00 (d, J = 1.96 Hz, 1H), 9.82 (s, 1H), 13.41 (br s, 1H). | Example 764 ethyl 5-{2-[(2- fluoro-5- methylphenyl)- amino]-4-[3- (trifluoromethyl)- 1H-pyrazol-1- yl]pyrimidin-5- yl}pyridine-3- carboxylate |
| Example 773^{h)} | 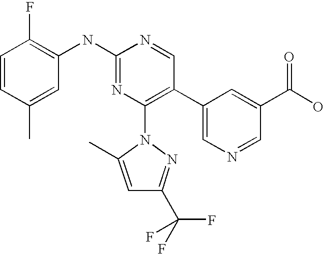 5-{2-[(2-fluoro-5-methylphenyl)-amino]-4-[5- methyl-3-(trifluoromethyl)-1H-pyrazol-1- yl]pyrimidin-5-yl}pyridine-3-carboxylic acid | MS(ES): 473 (M + 1) for C₂₂H₁₆F₄N₆O₂. 400 MHz, DMSO-d₆: δ 2.30 (s, 3H), 2.38 (s, 3H), 6.72 (s, 1H), 7.06-7.07 (m, 1H), 7.18 (dd, J = 8.40, 10.80 Hz, 1H), 7.42 (d, J = 6.40 Hz, 1H), 7.83 (t, J = 2.00 Hz, 1H), 8.53 (d, J = 2.00 Hz, 1H), 8.87 (s, 1H), 8.95 (d, J = 2.00 Hz, 1H), 9.87 (s, 1H), 13.43 (s, 1H). | Example 765 ethyl 5-{2-[(2- fluoro-5- methylphenyl)- amino]-4-[5- methyl-3- (trifluoromethyl)- 1H-pyrazol-1- yl]pyrimidin-5- yl}pyridine-3- carboxylate |
| Example 774ⁱ⁾ | 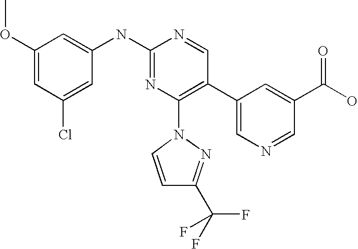 5-{2-[(3-chloro-5-methoxyphenyl)-amino]-4-[3- (trifluoromethyl)-1H-pyrazol-1-yl]pyrimidin-5- yl}pyridine-3-carboxylic acid | MS(ES): 491 (M + 1) for C₂₁H₁₄ClF₃N₆O₃. 400 MHz, DMSO-d₆: δ 3.78 (s, 3H), 6.70 (s, 1H), 7.05 (s, 1H), 7.47 (s, 1H), 7.52 (s, 1H), 8.02 (s, 1H), 8.50 (s, 1H), 8.62 (s, 1H), 8.85 (s, 1H), 9.00 (s, 1H), 10.43 (s, 1H), 13.55 (s, 1H). | Example 766 ethyl 5-{2-[(3- chloro-5- methoxyphenyl)- amino]-4-[3- (trifluoromethyl)- 1H-pyrazol-1- yl]pyrimidin-5- yl}pyridine-3- carboxylate |
| Example 775ⁱ⁾ | 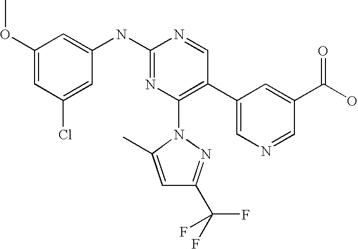 5-{2-[(3-chloro-5-methoxyphenyl)-amino]-4-[5- methyl-3-(trifluoromethyl)-1H-pyrazol-1- yl]pyrimidin-5-yl}pyridine-3-carboxylic acid | MS(ES): 505 (M + 1) for C₂₂H₁₆ClF₃N₆O₃. 400 MHz, DMSO-d₆: δ 2.42 (s, 3H), 3.76 (s, 3H), 6.71 (d, J = 1.80 Hz, 1H), 6.77 (s, 1H), 7.37 (s, 1H), 7.53 (s, 1H), 7.83 (t, J = 2.00 Hz, 1H), 8.55 (d, J = 2.12 Hz, 1H), 8.96 (d, J = 1.84 Hz, 1H), 9.01 (s, 1H), 10.45 (s, 1H), 13.44 (s, 1H). | Example 767 ethyl 5-{2-[(3- chloro-5- methoxyphenyl)- amino]-4-[5- methyl-3- (trifluoromethyl)- 1H-pyrazol-1- yl]pyrimidin-5- yl}pyridine-3- carboxylate |
| Example 776^{h)} | 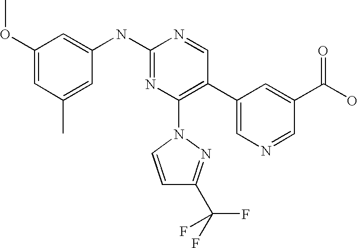 5-{2-[(3-methoxy-5-methylphenyl)amino]-4-[3- (trifluoromethyl)-1H-pyrazol-1-yl]pyrimidin-5- yl}pyridine-3-carboxylic acid | MS(ES): 471 (M + 1) for C₂₂H₁₇F₃N₆O₃. 400 MHz, DMSO-d₆: δ 2.28 (s, 3H), 3.74 (s, 3H), 6.47 (s, 1H), 7.03 (d, J = 2.60 Hz, 1H), 7.16 (s, 1H), 7.35 (s, 1H), 8.01 (t, J = 1.92 Hz, 1H), 8.52 (d, J = 1.52 Hz, 1H), 8.62 (s, 1H), 8.79 (s, 1H), 8.99 (d, J = 1.44 Hz, 1H), 10.18 (s, 1H), 13.41 (s, 1H). | Example 768 ethyl 5-{2-[(3- methoxy-5- methylphenyl)- amino]-4-[3- (trifluoromethyl)- 1H-pyrazol-1- yl]pyrimidin-5- yl}pyridine-3- carboxylate |
| Example 777^{h)} | 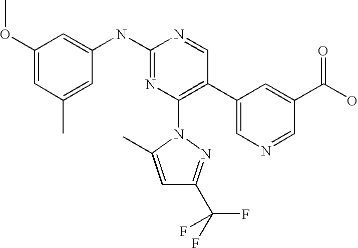 5-{2-[(3-methoxy-5-methylphenyl)amino]-4-[5- methyl-3-(trifluoromethyl)-1H-pyrazol-1- yl]pyrimidin-5-yl}pyridine-3-carboxylic acid | MS(ES): 485 (M + 1) for C₂₃H₁₉F₃N₆O₃. 400 MHz, DMSO-d₆: δ 2.27 (s, 3H), 2.42 (s, 3H), 3.72 (s, 3H), 6.47 (s, 1H), 6.76 (s, 1H), 7.13 (s, 1H), 7.27 (s, 1H), 7.81 (d, J = 1.84 Hz, 1H), 8.53 (d, J = 1.92 Hz, 1H), 8.95 (m, 2H), 10.21 (s, 1H), 13.45 (s, 1H). | Example 769 ethyl 5-{2-[(3- methoxy-5- methylphenyl)- amino]-4-[5- methyl-3- (trifluoromethyl)- 1H-pyrazol-1- yl]pyrimidin-5- yl}pyridine-3- carboxylate |

^{f)}overnight, RT,
^{g)}6 h, RT,
^{h)}40° C., 2 h,
ⁱ⁾50° C., 45 min

### Example 778

### methyl 5-{2-[(3-cyano-5-methylphenyl)amino]-4-[3-(trifluoromethyl)-1H-pyrazol-1-yl]pyrimidin-5-yl}-2-methoxypyridine-3-carboxylate

### Example 779

### methyl 5-{2-[(3-cyano-5-methylphenyl)amino]-4-[5-methyl-3-(trifluoromethyl)-1H-pyrazol-1-yl]pyrimidin-5-yl}-2-methoxypyridine-3-carboxylate

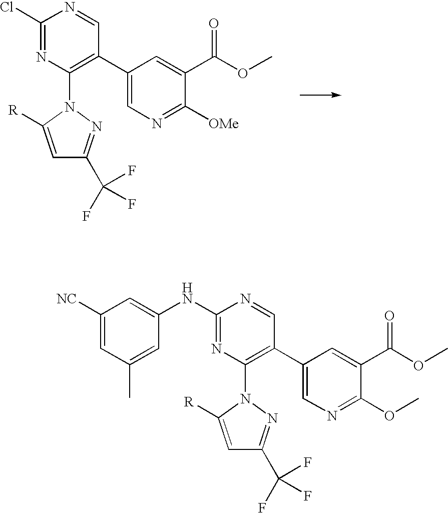

A suspension of methyl 5-{2-chloro-4-[3-(trifluoromethyl)-1H-pyrazol-1-yl]pyrimidin-5-yl}-2-methoxypyridine-3-carboxylate (Intermediate 325, 1 eq) or methyl 5-{2-chloro-4-[5-methyl-3-(trifluoromethyl)-1H-pyrazol-1-yl]pyrimidin-5-yl}-2-methoxypyridine-3-carboxylate (Intermediate 273, 1 eq), 3-amino-5-methylbenzonitrile (1.0 eq), tris(dibenzylideneacetone)dipalladium(0) (10 mol %), 2-dicyclohexylphosphino-2′,4′,6′-triisopropyl-1,1′-biphenyl (20 mol %) and sodium carbonate (1 eq) in acetonitrile/water (5:1) was heated to 90° C. for 30 min. The reaction mixture was concentrated. The residue was taken in ethyl acetate, washed with water and brine. The organic layer was separated, dried over sodium sulfate, filtered and concentrated. The crude mass was purified by silica gel column chromatography (60-120 mesh) using ethyl acetate/hexanes as an eluent to give the product. The compounds in the below table were prepared using this procedure and the specified starting material.

| Compound | Structure | Mass spectrum and ¹H NMR | SM |
|---|---|---|---|
| Example 778 | 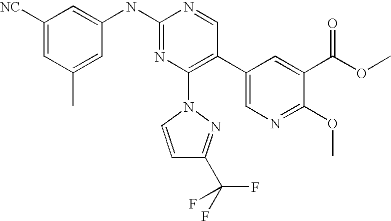 methyl 5-{2-[(3-cyano-5-methylphenyl)-amino]- 4-[3-(trifluoromethyl)-1H-pyrazol-1-yl]pyrimidin- 5-yl}-2-methoxypyridine-3-carboxylate | MS(ES): 510 (M + 1) for C₂₄H₁₈F₃N₇O₃. 400 MHz, DMSO-d₆: δ 2.34 (s, 3H), 3.77 (s, 3H), 3.92 (s, 3H), 7.04 (s, 1H), 7.33 (s, 1H), 7.77 (m, 2H), 8.11 (s, 1H), 8.30 (d, J = 2.24 Hz, 1H), 8.51 (s, 1H), 8.84 (s, 1H), 9.16 (s, 1H). | Intermediate 325 methyl 5-{2- chloro-4-[3- (trifluoromethyl)- 1H-pyrazol-1- yl]pyrimidin-5-yl}- 2-methoxypyridine- 3-carboxylate |
| Example 779 | 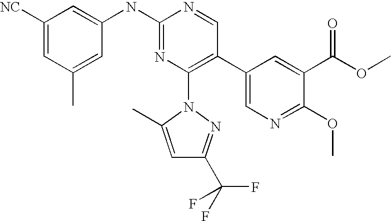 methyl 5-{2-[(3-cyano-5-methylphenyl)amino]- 4-[5-methyl-3-(trifluoromethyl)-1H-pyrazol-1- yl]pyrimidin-5-yl}-2-methoxypyridine-3-carboxylate | MS(ES): 524 (M + 1) for C₂₅H₂₀F₃N₇O₃. 400 MHz, DMSO-d₆: δ 2.35 (s, 3H), 2.35 (s, 3H), 3.76 (s, 3H), 3.93 (s, 3H), 6.79 (s, 1H), 7.34 (s, 1H), 7.62 (d, J = 2.56 Hz, 1H), 7.81 (s, 1H), 8.10 (s, 1H), 8.25 (d, J = 2.56 Hz, 1H), 9.01 (s, 1H), 10.55 (s, 1H). | Intermediate 273 methyl 5-{2-chloro- 4-[5-methyl-3- (trifluoromethyl)- 1H-pyrazol-1- yl]pyrimidin- 5-yl}-2- methoxy-pyridine- 3-carboxylate |

### Example 780

### 5-{2-[(3-cyano-5-methylphenyl)amino]-4-[3-(trifluoromethyl)-1H-pyrazol-1-yl]pyrimidin-5-yl}-2-methoxypyridine-3-carboxylic acid

### Example 781

### 5-{2-[(3-cyano-5-methylphenyl)amino]-4-[5-methyl-3-(trifluoromethyl)-1H-pyrazol-1-yl]pyrimidin-5-yl}-2-methoxypyridine-3-carboxylic acid

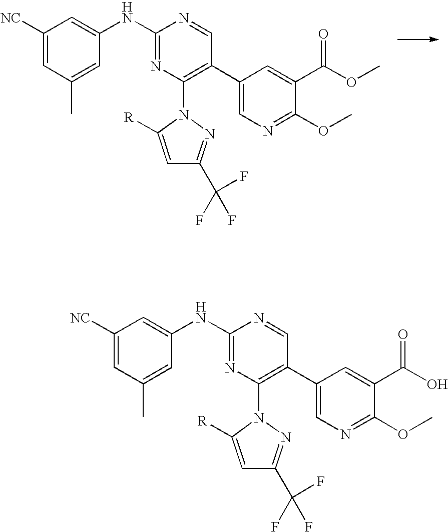

To 1 eq of methyl 5-{2-[(3-cyano-5-methylphenyl)amino]-4-[1H-azol-1-yl]pyrimidin-5-yl}-2-methoxypyridine-3-carboxylate taken in dioxane (5 mL), was added 1 N aq. sodium hydroxide (1-3 eqs) and stirred at RT for 1 h. The reaction mixture was carefully acidified with 1 N HCl and the solid that precipitated was filtered, washed with water and dried in vacuo to yield the carboxylic acid product. The compounds in the below table were prepared using this procedure and the specified starting material.

| Compound | Structure | Mass spectrum and ¹H NMR | SM |
|---|---|---|---|
| Example 780^{j)} | 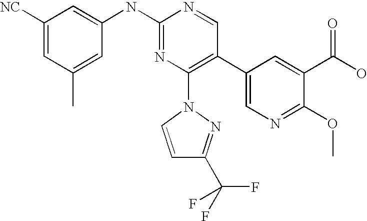 5-{2-[(3-cyano-5-methylphenyl)-amino]-4-[3- (trifluoromethyl)-1H-pyrazol-1-yl]pyrimidin-5- yl}-2-methoxypyridine-3-carboxylic acid | MS(ES): 496 (M + 1) for C₂₃H₁₆F₃N₇O_{3.} 400 MHz, DMSO-d₆P: δ 2.35 (s, 3H), 3.91 (s, 3H), 7.03 (d, J = 2.56 Hz, 1H), 7.31 (s, 1H), 7.79 (d, J = 2.20 Hz, 1H), 7.87 (s, 1H), 8.10 (s, 1H), 8.18 (s, 1H), 8.46 (s, 1H), 8.81 (s, 1H), 10.50 (s, 1H). | Example 778 methyl 5-{2-[(3- cyano-5- methylphenyl)- amino]-4-[3- (trifluoro-methyl)- 1H-pyrazol-1- yl]pyrimidin-5-yl}- 2-methoxy-pyridine- 3-carboxylate |
| Example 781^{k)} | 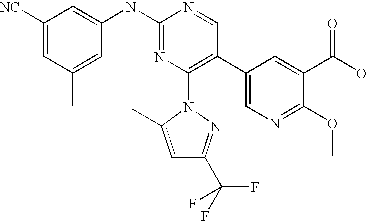 5-{2-[(3-cyano-5-methylphenyl)-amino]-4-[5- methyl-3-(trifluoromethyl)-1H-pyrazol-1- yl]pyrimidin-5-yl}-2-methoxypyridine-3-carboxylic acid | MS(ES): 510 (M + 1) for C₂₄H₁₈F₃N₇O_{3.} 400 MHz, DMSO-d₆: δ 2.35 (s, 6H), 3.89 (s, 3H), 6.77 (s, 1H), 7.33 (s, 1H), 7.61 (d, J = 2.36 Hz, 1H), 7.81 (s, 1H), 8.10 (m, 2H), 8.99 (s, 1H), 10.54 (s, 1H), 12.99 (s, 1H). | Example 779 methyl 5-{2-[(3- cyano-5- methylphenyl)- amino]-4-[5- methyl-3- (trifluoromethyl)- 1H-pyrazol-1- yl]pyrimidin-5-yl}- 2-methoxypyridine- 3-carboxylate |

^{j)}(2 eq NaOH),
^{k)}(3 eq NaOH).

### Example 782

### ethyl 5-{2-[(3-cyano-5-methylphenyl)amino]-4-[3-(trifluoromethyl)-1H-pyrazol-1-yl]pyrimidin-5-yl}pyridine-3-carboxylate

### Example 783

### ethyl 5-{2-[(3-cyano-5-methylphenyl)amino]-4-[5-methyl-3-(trifluoromethyl)-1H-pyrazol-1-yl]pyrimidin-5-yl}pyridine-3-carboxylate

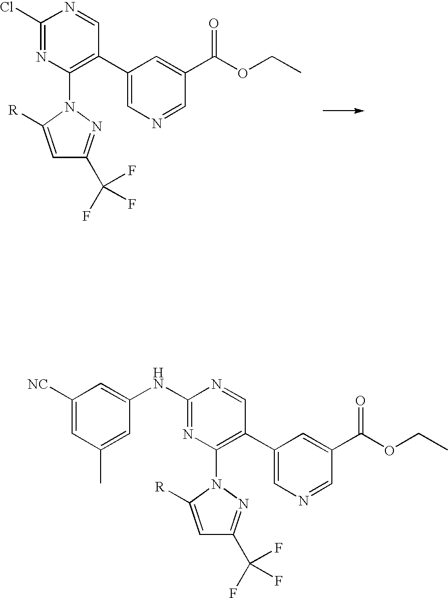

A suspension of ethyl 5-{2-chloro-4-[3-(trifluoromethyl)-1H-pyrazol-1-yl]pyrimidin-5-yl}pyridine-3-carboxylate (Intermediate 270, 1 eq) or ethyl 5-{2-chloro-4-[5-methyl-3-(trifluoromethyl)-1H-pyrazol-1-yl]pyrimidin-5-yl}pyridine-3-carboxylate (Intermediate 271, 1 eq), 3-amino-5-methylbenzonitrile (1.2 eq), tris(dibenzylideneacetone)dipalladium(0) (10 mol %), 2-dicyclohexylphosphino-2′,4′,6′-triisopropyl-1,1′-biphenyl (20 mol %) and sodium carbona eq) in acetonitrile/water (5:1) was heated to 90° C. for 30 min. The reaction mixture was concentrated. The residue taken in ethyl acetate was washed with water and brine. The organic layer was separated, dried over sodium sulfate, filtered and concentrated. The crude mass was purified by silica gel column chromatography (60-120 mesh) using ethyl acetate/hexanes as an eluent to give the product. The compounds in the below table were prepared using this procedure and the specified starting material.

| Compound | Structure | Mass spectrum and ¹H NMR | SM |
|---|---|---|---|
| Example 782 | 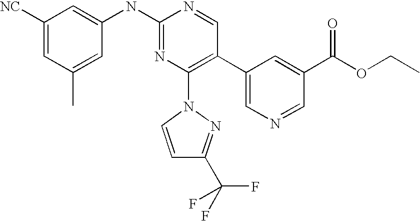 ethyl 5-{2-[(3-cyano-5-methylphenyl)-amino]-4-[3- (trifluoromethyl)-1H-pyrazol-1-yl]pyrimidin-5- yl}pyridine-3-carboxylate | MS(ES): 494 (M + 1) for C₂₄H₁₈F₃N₇O_{2.} 400 MHz, DMSO-d₆: δ 1.27 (t, J = 7.12 Hz, 3H), 2.36 (s, 3H), 4.33 (q, J = 7.16 Hz, 2H), 7.06 (d, J = 2.64 Hz, 1H), 7.33 (s, 1H), 7.87 (s, 1H), 8.04 (s, 1H), 8.10 (s, 1H), 8.55 (d, J = 1.88 Hz, 1H), 8.69 (d, J = 2.00 Hz, 1H), 8.87 (s, 1H), 9.03 (s, 1H), 10.56 (s, 1H). | Intermediate 270 ethyl 5-{2-chloro- 4-[3- (trifluoromethyl)- 1H-pyrazol-1- yl]pyrimidin-5- yl}pyridine-3- carboxylate |
| Example 783 | 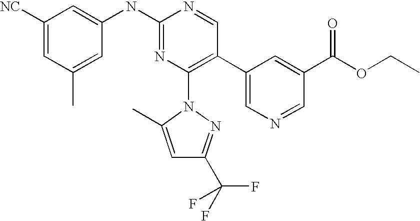 ethyl 5-{2-[(3-cyano-5-methylphenyl)amino]-4-[5-methyl-3- (trifluoromethyl)-1H-pyrazol-1-yl]pyrimidin-5-yl}pyridine-3- carboxylate | MS(ES): 508 (M + 1) for C₂₅H₂₀F₃₄N₇O_{2.} 300 MHz, DMSO-d₆: δ 1.29 (t, J = 7.08 Hz, 3H), 2.34 (s, 3H), 2.39 (s, 3H), 4.30 (q, J = 6.00 Hz, 2H), 6.66 (m, 1H), 6.78 (s, 1H), 7.33 (s, 1H), 7.80 (m, 2H), 8.09 (s, 1H), 8.60 (s, 1H), 8.98 (s, 1H), 9.03 (s, 1H). | Intermediate 271 ethyl 5-{2-chloro- 4-[5-methyl-3- (trifluoromethyl)- 1H-pyrazol-1- yl]pyrimidin-5- yl}pyridine-3- carboxylate |

### Example 784

### 5-{2-[(3-cyano-5-methylphenyl)amino]-4-[3-(trifluoromethyl)-1H-pyrazol-1-yl]pyrimidin-5-yl}pyridine-3-carboxylic acid

### Example 785

### 5-{2-[(3-cyano-5-methylphenyl)amino]-4-[5-methyl-3-(trifluoromethyl)-1H-pyrazol-1-yl]pyrimidin-5-yl}pyridine-3-carboxylic acid

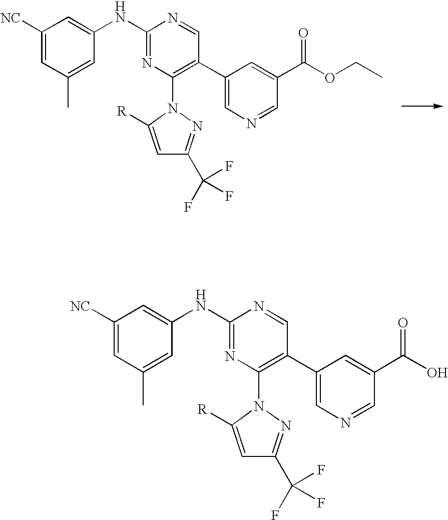

To 1 eq of ethyl 5-{2-[(3-cyano-5-methylphenyl)amino]-4-[1H-azol-1-yl]pyrimidin-5-yl}pyridine-3-carboxylate taken in dioxane (5 mL), was added 1 N aq. sodium hydroxide (1-3 eq) and stirred at RT for 1 h. After completion of reaction, reaction mixture was carefully acidified with 1 N HCl and the solid that precipitated was filtered, washed with water and dried in vacuo to yield the carboxylic acid product. The compounds in the below table were prepared using this procedure and the specified starting material.

| Compound | Structure | Mass spectrum and ¹H NMR | SM |
|---|---|---|---|
| Example 784^{l)} | 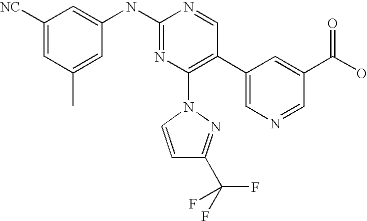 5-{2-[(3-cyano-5-methylphenyl)-amino]-4-[3- (trifluoromethyl)-1H-pyrazol-1-yl]pyrimidin-5- yl}pyridine-3-carboxylic acid | MS(ES): 466 (M + 1) for C₂₂H₁₄F₃N₇O_{2.} 400 MHz, DMSO-d₆: δ 2.36 (s, 3H), 7.04 (s, 1H), 7.32 (s, 1H), 7.88 (s, 1H), 8.02 (s, 1H), 8.10 (s, 1H), 8.51 (s, 2H), 8.84 (s, 1H), 8.99 (s, 1H), 10.55 (s, 1H). | Example 782 ethyl 5-{2-[(3- cyano-5- methylphenyl)- amino]-4-[3- (trifluoromethyl)- 1H-pyrazol-1- yl]pyrimidin-5- yl}pyridine-3- carboxylate |
| Example 785^{m)} | 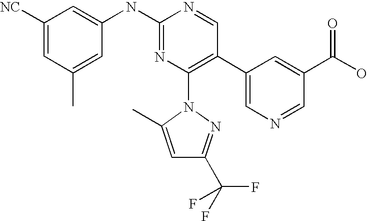 5-{2-[(3-cyano-5-methylphenyl)-amino]-4-[5- methyl-3-(trifluoromethyl)-1H-pyrazol-1- yl]pyrimidin-5-yl}pyridine-3-carboxylic acid | MS(ES): 480 (M + 1) for C₂₃H₁₆F₃N₇O_{2.} 400 MHz, DMSO-d₆: δ 2.36 (s, 3H), 2.40 (s, 3H), 6.76 (s, 1H), 7.33 (s, 1H), 7.82 (s, 1H), 7.92 (s, 1H), 8.10 (s,1H), 8.30 (s, 1H), 8.95 (s, 1H), 8.99 (s, 1H), 10.56 (s, 1H). | Example 783 ethyl 5-{2-[(3- cyano-5- methylphenyl)- amino]-4-[5-methyl- 3-(trifluoromethyl)- 1H-pyrazol-1- yl]pyrimidin-5- yl}pyridine-3- carboxylate |

^{l)}(1 eq NaOH),
^{m)}(3 eq NaOH).

### Example 786

### methyl 5-{2-[(3-chloro-4-fluorophenyl)amino]-4-[5-methyl-3-(trifluoromethyl)-1H-pyrazol-1-yl]pyrimidin-5-yl}-2-[(1-methylpyrrolidin-3-yl)oxy]pyridine-3-carboxylate

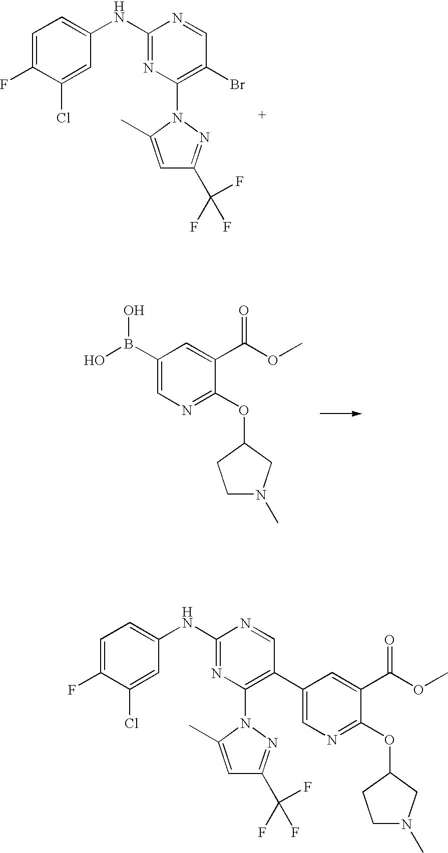

A suspension of 5-bromo-N-(3-chloro-4-fluorophenyl)-4-[5-methyl-3-(trifluoromethyl)-1H-pyrazol-1-yl]pyrimidin-2-amine (Intermediate 113, 0.75 mmol, 0.340 g), a mixture of {5-(methoxycarbonyl)-6-[(1-methylpyrrolidin-3-yl)oxy]pyridin-3-yl}boronic acid and methyl 2-[methylpyrrolidin-3-yl)oxy]-5-(4,4,5,5-tetramethyl-1,3,2-dioxaborolan-2-yl)pyridine-3-carboxylate (Intermediate 297, 1.5 mmol based on the boronic acid, 0.423 g), [1,1′-bis(diphenylphosphino)ferrocene]dichloropalladium(II) (0.15 mmol, 0.109 g) and sodium carbonate (0.75 mmol, 0.074 g) in acetonitrile/water (5:1) was degassed and heated to 90° C. for 15 min under an inert atmosphere. The reaction mass was passed through a celite bed and solvent was concentrated in vacuo. The resultant residue taken in EtOAc (50 mL) was washed with water, brine, dried over Na₂SO₄, filtered and concentrated. The crude mass was further purified by silica gel column chromatography (eluted with 3% Et₃N in EtOAc) to yield 0.210 g of the product.

| | | Mass spectrum and ¹H | |
|---|---|---|---|
| Compound | Structure | NMR | SM |
| Example 786 | 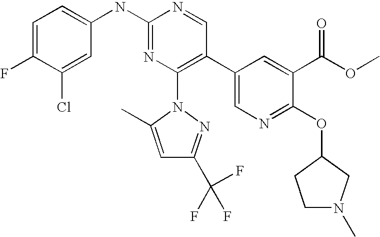 methyl 5-{2-[(3- chloro-4- fluorophenyl)amino]- 4-[5-methyl-3- (trifluoromethyl)-1H- pyrazol-1- yl]pyrimidin-5-yl}-2- [(1-methylpyrrolidin-3- yl)oxy]pyridine-3- carboxylate | MS(ES): 606 (M + 1) for C₂₇H₂₄ClF₄N₇O₃. 400 MHz, DMSO-d₆: δ 1.78 (br s, 1H), 2.24 (s, 3H), 2.27 (d, J = 5.48 Hz, 1H), 2.32 (s, 3H), 2.55 (dd, J = 2.08, 10.80 Hz, 1H), 2.65 (t, J = 6.52 Hz, 1H), 2.79 (dd, J = 6.20, 10.50 Hz, 1H), 3.74 (s, 3H), 5.36 (br s, 1H), 6.77 (s, 1H), 7.42 (t, J = 9.12 Hz, 1H), 7.61-7.66 (m, 2H), 8.05 (d, J = 4.40 Hz, 1H), 8.14 (d, J = 2.48 Hz, 1H), 8.95 (s, 1H), 10.44 (s, 1H). | Intermediate 297 {5- (methoxy- carbonyl)-6- [(1- methyl- pyrrolidin-3- yl)oxy]- pyridin-3- yl}boronic acid |

### Example 787

### 5-{2-[(3-chloro-4-fluorophenyl)amino]-4-[5-methyl-3-(trifluoromethyl)-1H-pyrazol-1-yl]pyrimidin-5-yl}-2-[(1-methylpyrrolidin-3-yl)oxy]pyridine-3-carboxylic acid

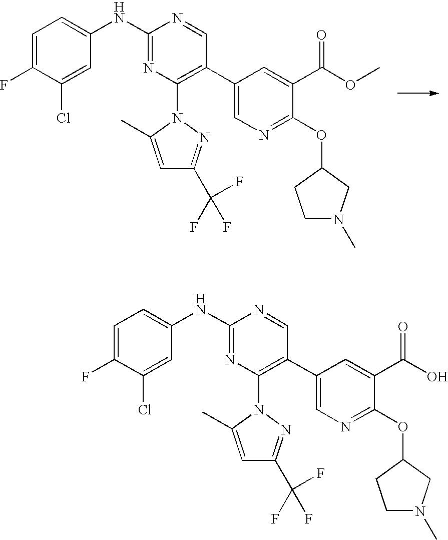

To 130 mg of methyl 5-{2-[(3-chloro-4-fluorophenyl)amino]-4-[5-methyl-3-(trifluoromethyl)-1H-pyrazol-1-yl]pyrimidin-5-yl}-2-[(1-methylpyrrolidin-3-yl)oxy]pyridine-3-carboxylate (Example 786, 0.2 mmol) taken in a mixture of THF (5 mL) and water (5 mL), was added Barium hydroxide monohydrate (0.162 g, 0.8 mmol) and stirred at room temperature for 7 h. The mixture was carefully acidified with 1 N HCl. It was extracted with ethyl acetate (50 mL) and the organic layer was washed with water and brine, dried over Na₂SO₄ and concentrated to yield 85 mg of Example 787.

| | | Mass spectrum and ¹H | |
|---|---|---|---|
| Compound | Structure | NMR | SM |
| Example 787 | 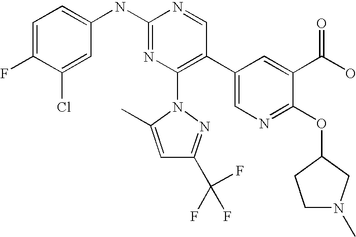 5-{2-[(3-chloro-4- fluorophenyl)amino]- 4-[5-methyl-3- (trifluoromethyl)-1H- pyrazol-1- yl]pyrimidin-5-yl}-2- [(1-methylpyrrolidin-3- yl)oxy]pyridine-3- carboxylic acid | MS(ES): 592 (M + 1) for C₂₆H₂₂ClF₄N₇O₃. 400 MHz, DMSO-d₆: δ 1.92- 1.95 (m, 1H), 2.28-2.29 (m, 1H), 2.35 (s,3H), 2.43 (s, 3H), 2.64 (d, J = 7.00 Hz, 1H), 2.76-2.79 (m, 1H), 2.92-2.95 (m, 1H), 3.00-3.01 (m, 1H), 5.41 (s, 1H), 6.75 (s, 1H), 7.41 (t, J = 9.08 Hz, 1H), 7.55 (s, 1H), 7.59-7.65 (m, 1H), 7.94 (s, 1H), 8.05 (d, J = 4.60 Hz, 1H), 8.91 (s, 1H), 10.42 (s, 1H). | Example 786 methyl 5- {2-[(3- chloro-4- fluoro- phenyl)amino]-4- [5-methyl- 3- (trifluoro- methyl)-1H- pyrazol-1- yl]pyrimidin- 5-yl}-2-[(1- methyl- pyrrolidin-3- yl)oxy]- pyridine-3- carboxylate |

### Example 788

### methyl 5-{2-[(3-chloro-4-fluorophenyl)amino]-4-[3-(trifluoromethyl)-1H-pyrazol-1-yl]pyrimidin-5-yl}-2-[(1-methylpyrrolidin-3-yl)oxylpyridine-3-carboxylate

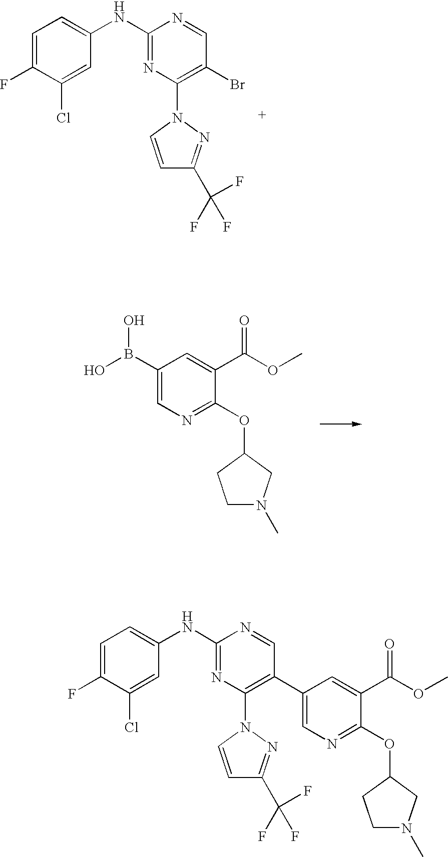

A suspension of 5-bromo-N-(3-chloro-4-fluorophenyl)-4-[3-(trifluoromethyl)-1H-pyrazol-1-yl]pyrimidin-2-amine (Intermediate 115, 1.1 mmol, 0.5 g), a mixture of {5-(methoxycarbonyl)-6-[(1-methylpyrrolidin-3-yl)oxy]pyridin-3-yl}boronic acid and methyl 2-[methylpyrrolidin-3-yl)oxy]-5-(4,4,5,5-tetramethyl-1,3,2-dioxaborolan-2-yl)pyridine-3-carboxylate (Intermediate 297, 2.3 mmol based on the boronic acid, 0.644 g), [1,1′-bis(diphenylphosphino)ferrocene]dichloropalladium(II) (0.22 mmol, 0.160 g) and sodium carbonate (1.1 mmol, 0.116 g) in acetonitrile/water (20:5, v/v) was degassed and heated to 90° C. for 15 min under an inert atmosphere. The reaction mass was passed through a celite bed. The solvent was concentrated in vacuo and the resultant crude mass was taken in EtOAc (50 mL), washed with water, brine, dried over Na₂SO₄, filtered and concentrated. The crude mass was further purified by silica gel column chromatography (eluted with 4% Et₃N in EtOAc) to yield 0.240 g of the product.

| | | Mass spectrum and ¹H | |
|---|---|---|---|
| Compound | Structure | NMR | SM |
| Example 788 | 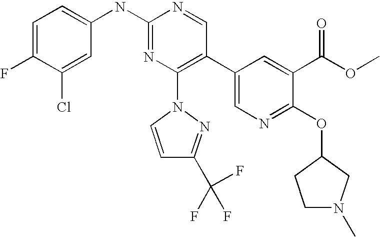 methyl 5-{2-[(3- chloro-4- fluorophenyl)amino]- 4-[3-(trifluoromethyl)- 1H-pyrazol-1- yl]pyrimidin-5-yl}-2- [(1-methylpyrrolidin-3- yl)oxy]pyridine-3- carboxylate | MS(ES): 592 (M + 1) for C₂₆H₂₂ClF₄N₇O₃. 400 MHz, DMSO-d₆: δ 1.81- 1.86 (m, 1H), 2.26 (s, 3H), 2.28-2.40 (m, 2H), 2.59 (dd, J = 2.76, 10.58 hz, 1H), 2.64-2.67 (m, 1H), 2.82 (dd, J = 6.24, 10.62 Hz, 1H), 3.81 (s, 3H), 5.40-5.44 (m, 1H), 7.05 (d, J = 2.60 Hz, 1H), 7.43 (t, J = 9.12 Hz, 1H), 7.71-7.75 (m, 1H), 7.87 (d, J = 2.44 Hz, 1H), 8.07 (dd, J = 2.56, 6.72 Hz, 1H), 8.21 (d, J = 2.48 Hz, 1H), 8.52 (d, J = 1.60 Hz, 1H), 8.80 (s, 1H), 10.42 (s, 1H). | Intermediate 297 {5- (methoxy- carbonyl)-6- [(1- methyl- pyrrolidin-3- yl)oxy]pyridin- 3- yl}boronic acid |

### Example 789

### 5-{2-[(3-chloro-4-fluorophenyl)amino]-4-[3-(trifluoromethyl)-1H-pyrazol-1-yl]pyrimidin-5-yl}-2-[(1-methylpyrrolidin-3-yl)oxy]pyridine-3-carboxylic acid

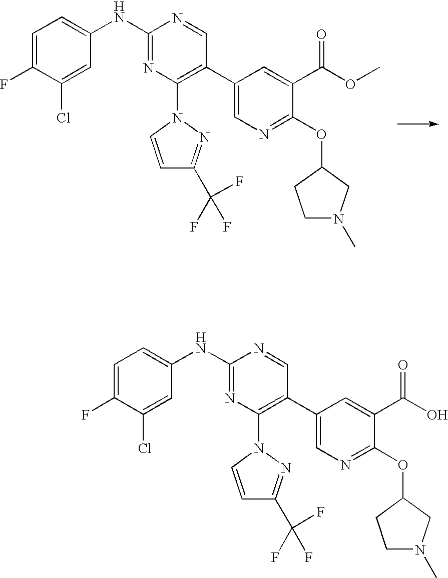

To 180 mg of methyl 5-{2-[(3-chloro-4-fluorophenyl)amino]-4-[3-(trifluoromethyl)-1H-pyrazol-1-yl]pyrimidin-5-yl}-2-[(1-methylpyrrolidin-3-yl)oxy]pyridine-3-carboxylate (Example 788, 0.3 mmol, 1 eq) taken in a mixture of THF (5 mL) and water (5 mL), was added Barium hydroxide monohydrate (0.231 g, 1.2 mmol, 4 eq) and stirred at room temperature for 7 h. The mixture was carefully acidified with 1 N HCl. It was extracted with ethyl acetate (50 mL) and the organic layer was washed with water and brine, dried over Na₂SO₄ and concentrated to yield 120 mg of the product.

| | | Mass spectrum and ¹H | |
|---|---|---|---|
| Compound | Structure | NMR | SM |
| Example 789 | 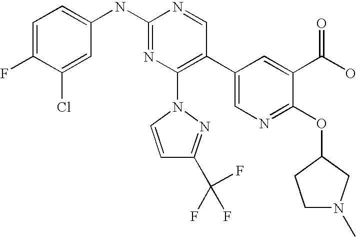 5-{2-[(3-chloro-4- fluorophenyl)amino]- 4-[3-(trifluoromethyl)- 1H-pyrazol-1- yl]pyrimidin-5-yl}-2- [(1-methylpyrrolidin-3- yl)oxy]pyridine-3- carboxylic acid | MS(ES): 578 (M + 1) for C₂₅H₂₀ClF₄N₇O₃. 400 MHz, DMSO-d₆: δ 1.95 (br s, 1H), 2.31 (br s, 1H), 2.43 (s, 3H), 2.65 (br s, 1H), 2.80 (d, J = 10.36 Hz, 1H), 2.96 (m, 2H), 5.44 (br s, 1H), 7.02 (s, 1H), 7.40 (t, J = 9.00 Hz, 1H), 7.7-7.73 (m, 2H), 8.05-8.07 (m, 2H), 8.47 (s, 1H), 8.76 (s, 1H), 10.40 (s, 1H). | Example 788 methyl 5- {2-[(3- chloro-4- fluoro- phenyl)amino]-4- [3- (trifluoro- methyl)-1H- pyrazol-1- yl]pyrimidin- 5-yl}-2-[(1- methyl- pyrrolidin- 3- yl)oxy] pyridine-3- carboxylate |

### Example 790

### methyl 5-{2-[(3-chloro-4-fluorophenyl)amino]-4-[5-methyl-3-(trifluoromethyl)-1H-pyrazol-1-yl]pyrimidin-5-yl}-2-[1-(pyridin-4-yl)ethoxy]pyridine-3-carboxylate

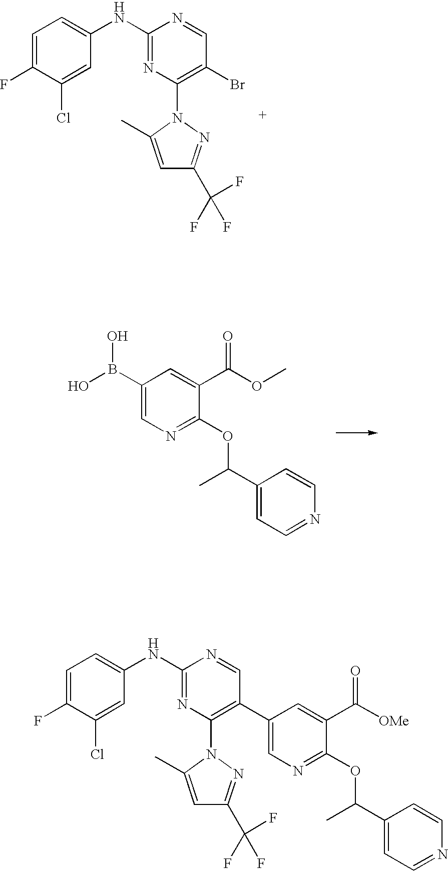

A suspension of 5-bromo-N-(3-chloro-4-fluorophenyl)-4-[5-methyl-3-(trifluoromethyl)-1H-pyrazol-1-yl]pyrimidin-2-amine (Intermediate 113, 0.97 mmol, 0.440 g), a mixture of methyl 2-[1-(pyridin-4-yl)ethoxy]-5-(4,4,5,5-tetramethyl-1,3,2-dioxaborolan-2-yl)pyridine-3-carboxylate and {5-(methoxycarbonyl)-6-[1-(pyridin-4-yl)ethoxy]pyridin-3-yl}boronic acid (Intermediate 300, 1.46 mmol based on the boronic acid, 0.442 g), [1,1′-bis(diphenylphosphino)ferrocene]dichloropalladium(II) complex with CH₂Cl₂ (0.2 mmol, 0.159 g) and sodium carbonate (0.97 mmol, 0.103 g) in acetonitrile/water (4:1) was degassed and heated to 90° C. for 30 min under an inert atmosphere. The reaction mixture was diluted with EtOAc (50 mL), washed with water and brine, dried over Na₂SO₄, filtered and concentrated. The crude material was purified by silica gel column chromatography (product eluted with 45% ethyl acetate/hexanes) to yield 0.440 g of product.

| | | Mass spectrum and ¹H | |
|---|---|---|---|
| Compound | Structure | NMR | SM |
| Example 790 | 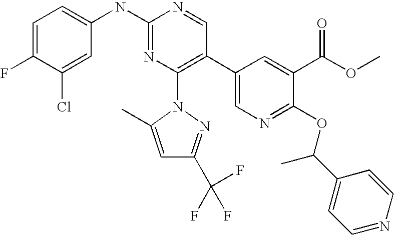 methyl 5-{2-[(3- chloro-4- fluorophenyl)amino]- 4-[5-methyl-3- (trifluoromethyl)-1H- pyrazol-1- yl]pyrimidin-5-yl}-2- [1-(pyridin-4- yl)ethoxy]pyridine-3- carboxylate | MS(ES): 628 (M + 1) for C₂₉H₂₂ClF₄N₇O₃. 400 MHz, DMSO-d₆: δ 1.54 (d, J = 8.72 Hz, 3H), 2.33 (s, 3H), 3.82 (s, 3H), 6.27 (q, J = 8.60 Hz, 1H), 6.74 (s, 1H), 7.37-7.40 (m, 3H), 7.61-7.62 (m, 1H), 7.68 (d, J = 3.32 Hz, 1H), 8.03 (dd, J = 3.12, 8.82 Hz, 1H), 8.09 (d, J = 3.36 Hz, 1H), 8.53 (s, 2H), 8.93 (s,1H), 10.43 (s, 1H). | Intermediate 300 {5- (methoxy- carbonyl)-6-[1- (pyridin-4- yl)ethoxy] pyridin-3- yl}boronic acid |

### Example 791

### 5-{2-[(3-chloro-4-fluorophenyl)amino]-4-[5-methyl-3-(trifluoromethyl)-1H-pyrazol-1-yl]pyrimidin-5-yl}-2-1H-(pyridin-4-yl)ethoxy]pyridine-3-carboxylic acid

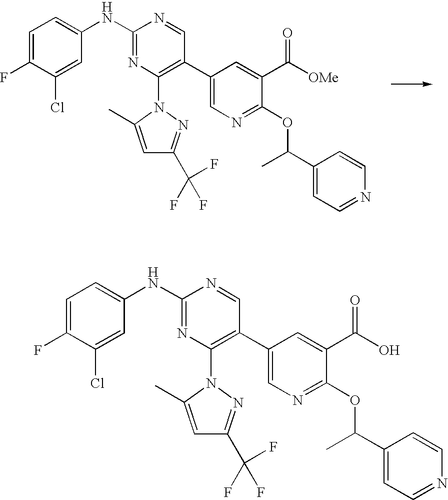

To 162 mg of methyl 5-{2-[(3-chloro-4-fluorophenyl)amino]-4-[5-methyl-3-(trifluoromethyl)-1H-pyrazol-1-yl]pyrimidin-5-yl}-2-[1-(pyridin-4-yl)ethoxy]pyridine-3-carboxylate (Example 790, 0.25 mmol, 0.162 g) taken in a mixture of dioxane (4 mL) and water (4 mL) was added Barium hydroxide monohydrate (0.51 mmol, 0.098 g) and stirred at 50° C. for 1 h. After completion of the reaction, the mixture was then carefully acidified with 1 N HCl. It was then extracted with ethyl acetate (50 mL), and the organic layer was washed with water and brine, dried over Na₂SO₄ and concentrated to yield 120 mg of the title compound.

| | | Mass spectrum and ¹H | |
|---|---|---|---|
| Compound | Structure | NMR | SM |
| Example 791 | 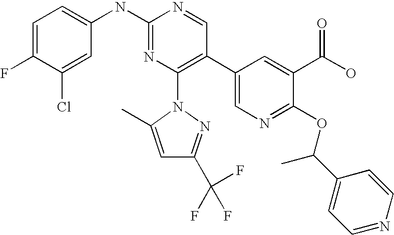 5-{2-[(3-chloro-4- fluorophenyl)amino]- 4-[5-methyl-3- (trifluoromethyl)-1H- pyrazol-1- yl]pyrimidin-5-yl}-2- [1-(pyridin-4- yl)ethoxy]pyridine-3- carboxylic acid | MS(ES): 614 (M + 1) for C₂₈H₂₀ClF₄N₇O₃. 400 Mhz, DMSO-d₆: δ 1.50 (d, J = 6.48 Hz, 3H), 2.25 (s, 3H), 6.20 (q, J = 6.48 Hz, 1H), 6.69 (s, 1H), 7.39 (t, J = 9.08 Hz, 1H), 7.51 (m, 3H), 7.59- 7.62 (m, 2H), 8.03 (dd, J = 2.32, 6.62 Hz, 1H), 8.46 (d, J = 4.60 Hz, 2H), 8.85 (s, 1H), 10.41 (s, 1H). | Example 790 methyl 5- {2-[(3- chloro-4- fluoro- phenyl)amino]-4- [5-methyl- 3- (trifluoro- methyl)-1H- pyrazol-1- yl]pyrimidin- 5-yl}-2-[1- (pyridin-4- yl)ethoxy] pyridine-3- carboxylate |

### Example 792

### methyl 5-{2-[(3-chloro-4-fluorophenyl)amino]-4-[3-(trifluoromethyl)-1H-pyrazol-1-yl]pyrimidin-5-yl}-2-[1-(pyridin-4-yl)ethoxy]pyridine-3-carboxylate

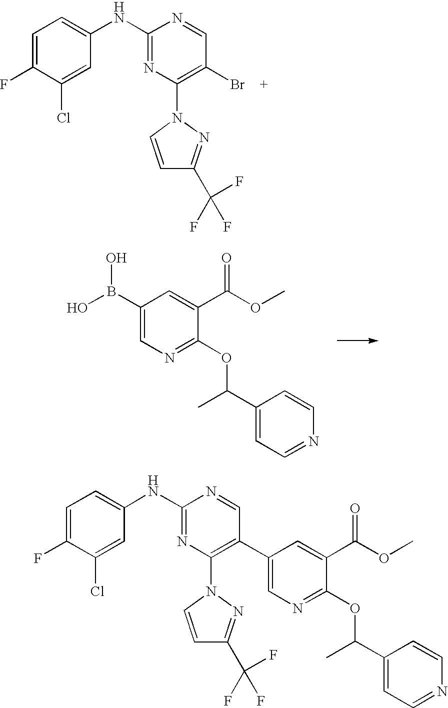

A suspension of 5-bromo-N-(3-chloro-4-fluorophenyl)-4-[3-(trifluoromethyl)-1H-pyrazol-1-yl]pyrimidin-2-amine (Intermediate 115, 0.95 mmol, 0.415 g), a mixture of methyl 2-[1-(pyridin-4-yl)ethoxy]-5-(4,4,5,5-tetramethyl-1,3,2-dioxaborolan-2-yl)pyridine-3-carboxylate and {5-(methoxycarbonyl)-6-[1-(pyridin-4-yl)ethoxy]pyridin-3-yl}boronic acid (Intermediate 300, 1.42 mmol based on the boronic acid, 0.431 g), [1,1′-bis(diphenylphosphino)ferrocene]dichloropalladium(II) complex with CH₂Cl₂ (0.19 mmol, 0.155 g) and sodium carbonate (0.95 mmol, 0.101 g) in acetonitrile/water (5:1) was degassed and heated to 90° C. for 30 min under an inert atmosphere. The solvent was concentrated in vacuo and the resultant crude mass was taken in EtOAc (50 mL), washed with water, brine, dried over Na₂SO₄, filtered and concentrated. The crude mass was purified by silica gel column chromatography (product eluted with 45% ethyl acetate/hexanes) to yield 0.415 g of the product.

| | | Mass spectrum and ¹H | |
|---|---|---|---|
| Compound | Structure | NMR | SM |
| Example 792 | 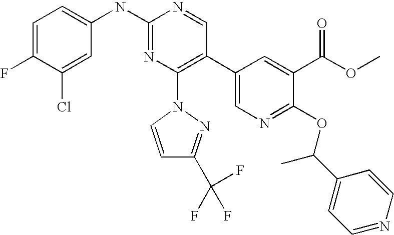 methyl 5-{2-[(3- chloro-4- fluorophenyl)amino]- 4-[3-(trifluoromethyl)- 1H-pyrazol-1- yl]pyrimidin-5-yl}-2- [1-(pyridin-4- yl)ethoxy]pyridine-3- carboxylate | MS(ES): 614 (M + 1) for C₂₈H₂₀ClF₄N₇O₃. 400 MHz, DMSO-d₆: δ 1.58 (d, J = 6.56 Hz, 3H), 3.85 (s, 3H), 6.33 (q, J = 6.52 Hz, 1H), 7.04 (d, J = 2.64 Hz, 1H), 7.43 (t, J = 9.12 Hz, 1H), 7.49 (d, J = 5.92 Hz, 2H), 7.70-7.71 (m, 1H), 7.94 (d, J = 2.44 Hz, 1H), 8.06 (dd, J = 2.56, 6.66 Hz, 1H), 8.18 (d, J = 2.44 Hz, 1H), 8.52 (s, 1H), 8.56 (m, 2H), 8.79 (s, 1H), 10.42 (s, 1H). | Intermediate 300 {5- (methoxy- carbonyl)-6-[1- (pyridin-4- yl)ethoxy] pyridin-3- yl}boronic acid |

### Example 793

### 5-{2-[(3-chloro-4-fluorophenyl)amino]-4-[3-(trifluoromethyl)-1H-pyrazol-1-yl]pyrimidin-5-yl}-2-[1-(pyridin-4-yl)ethoxy]pyridine-3-carboxylic acid

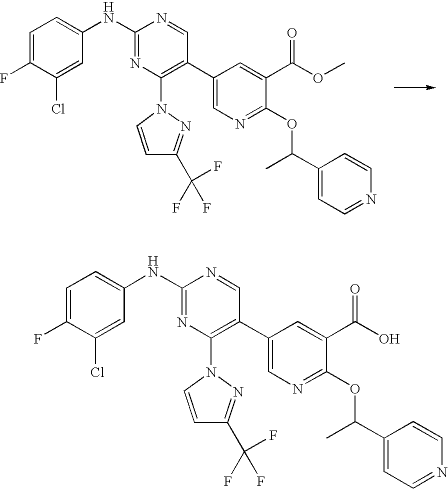

To 100 mg of methyl 5-{2-[(3-chloro-4-fluorophenyl)amino]-4-[3-(trifluoromethyl)-1H-pyrazol-1-pyrazol-1-yl]pyrimidin-5-yl}-2-[1-(pyridin-4-yl)ethoxy]pyridine-3-carboxylate (Example 792, 0.16 mmol) taken in a mixture of dioxane (4 mL) and water (4 mL), was added Barium hydroxide monohydrate (0.32 mmol, 0.062 g) and allowed to stir overnight at room temperature. The mixture was then carefully acidified with 1 N HCl and then diluted with ethyl acetate (50 mL), washed with water and brine, dried over Na₂SO₄ and concentrated. It was further purified by column chromatography using 1% MeOH in CHCl₃ to yield 0.080 g of the product.

| | | Mass spectrum and ¹H | |
|---|---|---|---|
| Compound | Structure | NMR | SM |
| Example 793 | 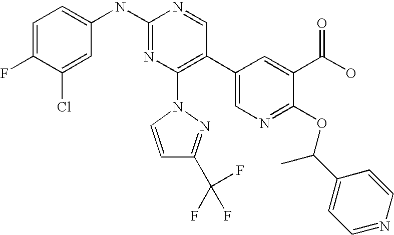 5-{2-[(3-chloro-4- fluorophenyl)amino]- 4-[3-(trifluoromethyl)- 1H-pyrazol-1- yl]pyrimidin-5-yl}-2- [1-(pyridin-4- yl)ethoxy]pyridine-3- carboxylic acid | MS(ES): 600 (M + 1) for C₂₇H₁₈ClF₄N₇O₃. 400 MHz, DMSO-d₆: δ 1.57 (d, J = 6.56 Hz, 3H), 6.31 (q, J = 6.48 Hz, 1H), 7.01 (d, J = 2.60 Hz, 1H), 7.41 (t, J = 9.08 Hz, 1H), 7.47 (d, J = 5.72 Hz, 2H), 7.68-7.72 (m, 1H), 7.87 (d, J = 2.40 Hz, 1H), 8.05 (dd, J = 2.48, 6.70 Hz, 1H), 8.11 (d, J = 2.28 Hz, 1H), 8.49 (s, 1H), 8.52 (d, J = 5.76 Hz, 2H), 8.76 (s, 1H), 10.40 (s, 1H), 13.09 (s, 1H). | Example 792 methyl 5- {2-[(3- chloro-4- fluorophenyl) amino]-4- [3- (trifluoro- methyl)-1H- pyrazol-1- yl]pyrimidin 5-yl}-2-[1- (pyridin-4- yl)ethoxy] pyridine-3- carboxylate |

### Example 794

### methyl 5-{2-[(3-chloro-4-fluorophenyl)amino]-4-[5-methyl-3-(trifluoromethyl)-1H-pyrazol-1-yl]pyrimidin-5-yl}-2-[2-(1H-imidazol-1-yl)ethoxy]pyridine-3-carboxylate

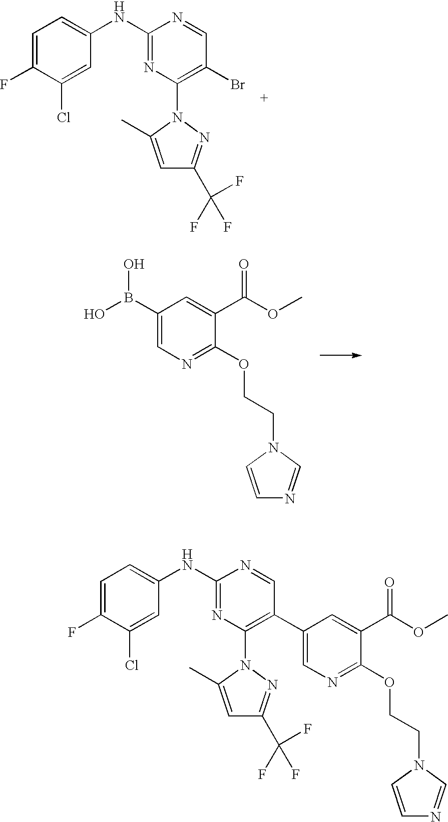

A suspension of 5-bromo-N-(3-chloro-4-fluorophenyl)-4-[5-methyl-3-(trifluoromethyl)-1H-pyrazol-1-yl]pyrimidin-2-amine (Intermediate 113, 1.11 mmol, 0.5 g), a mixture of {6-[2-(1H-imidazol-1-yl)ethoxy]-5-(methoxycarbonyl)pyridin-3-yl}boronic acid and methyl 2-[2-(1H-imidazol-1-yl)ethoxy]-5-(4,4,5,5-tetramethyl-1,3,2-dioxaborolan-2-yl)pyridine-3-carboxylate (Intermediate 303, 1.66 mmol based on the boronic acid, 0.486 g), [1,1′-bis(diphenylphosphino)ferrocene]dichloropalladium(II) (0.22 mmol, 0.162 g) and sodium carbonate (1.1 mmol, 0.117 g) in acetonitrile/water (5:1) was degassed and heated to 90° C. for 1 h under an inert atmosphere. The solvent was concentrated in vacuo and the resultant crude mass taken in EtOAc (50 mL), was washed with water, brine, dried over Na₂SO₄, filtered and concentrated. The crude material was further purified by silica gel column chromatography (product eluted with 0.5% Et₃N in EtOAc) to yield 0.31 g of the product.

| | | Mass spectrum and ¹H | |
|---|---|---|---|
| Compound | Structure | NMR | SM |
| Example 794 | 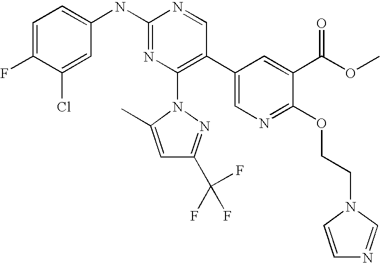 methyl 5-{2-[(3- chloro-4- fluorophenyl)amino]- 4-[5-methyl-3- (trifluoromethyl)-1H- pyrazol-1- yl]pyrimidin-5-yl}-2- [2-(1H-imidazol-1- yl)ethoxy]pyridine-3- carboxylate | MS(ES): 617 (M + 1) for C₂₇H₂₁ClF₄N₈O₃. 400 MHz, DMSO-d₆: δ 2.32 (s, 3H), 3.78 (s, 3H), 4.38 (t, J = 4.52 Hz, 2H), 4.55 (t, J = 5.00 Hz, 2H), 6.77 (s, 1H), 6.88 (s, 1H), 7.29 (s, 1H), 7.43 (t, J = 9.08 Hz, 1H), 7.63-7.68 (m, 2H), 7.69 (s, 1H), 8.07 (d, J = 4.60 Hz, 1H), 8.22 (d, J = 2.40 Hz, 1H), 8.96 (s, 1H), 10.47 (s, 1H). | Intermediate 303 {6-[2-(1H- imidazol-1- yl)ethoxy]- 5- (methoxy- carbonyl) pyridin-3- yl}boronic acid |

### Example 795

### 5-{2-[(3-chloro-4-fluorophenyl)amino]-4-[5-methyl-3-(trifluoromethyl)-1H-pyrazol-1-yl]pyrimidin-5-yl}-2-[2-(1H-imidazol-1-yl)ethoxy]pyridine-3-carboxylic acid

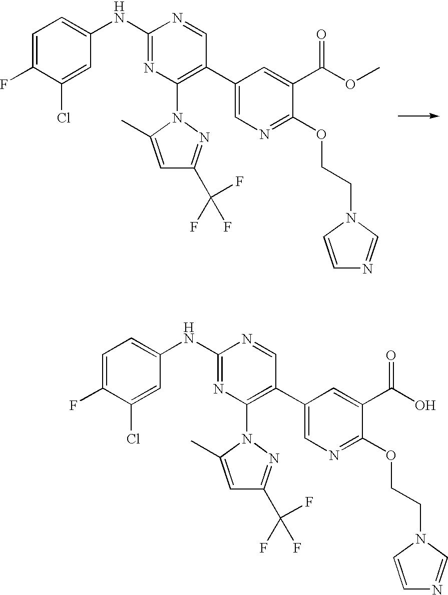

To 170 mg of methyl 5-{2-[(3-chloro-4-fluorophenyl)amino]-4-[5-methyl-3-(trifluoromethyl)-1H-pyrazol-1-yl]pyrimidin-5-yl}-2-[2-(1H-imidazol-1-yl)ethoxy]pyridine-3-carboxylate (Example 794, 0.27 mmol) taken in a mixture of dioxane (20 mL) and water (20 mL), was added Barium hydroxide monohydrate (0.27 mmol, 0.052 g) and the reaction mixture warmed to 50° C. for 24 h. Another equivalent of Barium hydroxide monohydrate (0.27 mmol, 0.052 g) was added and the reaction continued at 50° C. for 3 h more. The mixture was then carefully acidified with 1 N HCl. It was extracted with ethyl acetate (50 mL) and the organic layer was washed with water and brine, dried over Na₂SO₄ and concentrated to yield 0.112 g of Example 795.

| | | Mass spectrum and ¹H | |
|---|---|---|---|
| Compound | Structure | NMR | SM |
| Example 795 | 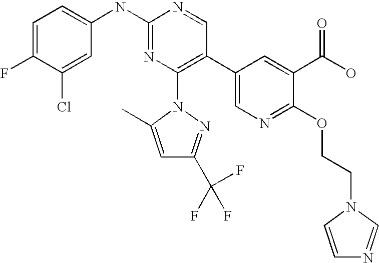 5-{2-[(3-chloro-4- fluorophenyl)amino]- 4-[5-methyl-3- (trifluoromethyl)-1H- pyrazol-1- yl]pyrimidin-5-yl}-2- [2-(1H-imidazol-1- yl)ethoxy]pyridine-3- carboxylic acid | MS(ES): 603 (M + 1) for C₂₆H₁₉ClF₄N₈O₃. 400 MHz, DMSO-d₆: δ 2.30 (s, 3H), 4.35 (s, 2H), 4.49 (s, 2H), 6.74 (s, 1H), 6.85 (s, 1H), 7.35 (s, 1H), 7.41 (t, J = 9.16 Hz, 1H), 7.63 (br s, 2H), 7.73 (s, 1H), 8.00 (s, 1H), 8.05 (d, J = 4.52 Hz, 1H), 8.92 (s, 1H), 10.43 (s, 1H). | Example 794 methyl 5- {2-[(3- chloro-4- fluorophenyl)- amino]-4- [5-methyl- 3- (trifluoro- methyl)-1H- pyrazol-1- yl]pyrimidin- 5-yl}-2-[2- (1H- imidazol-1- yl)ethoxy] pyridine-3- carboxylate |

### Example 796

### methyl 5-{2-[(3-chloro-4-fluorophenyl)amino]-4-[3-(trifluoromethyl)-1H-pyrazol-1-yl]pyrimidin-5-yl}-2-[2-(1H-imidazol-1-yl)ethoxy]pyridine-3-carboxylate

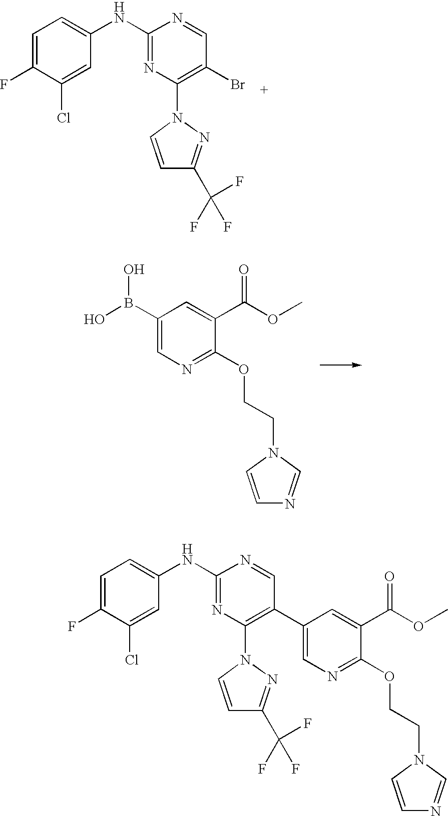

A suspension of 5-bromo-N-(3-chloro-4-fluorophenyl)-4-[3-(trifluoromethyl)-1H-pyrazol-1-yl]pyrimidin-2-amine (Intermediate 115, 1.14 mmol, 0.5 g), a mixture of {6-[2-(1H-imidazol-1-yl)ethoxy]-5-(methoxycarbonyl)pyridin-3-yl}boronic acid and methyl 2-[2-(1H-imidazol-1-yl)ethoxy]-5-(4,4,5,5-tetramethyl-1,3,2-dioxaborolan-2-yl)pyridine-3-carboxylate (Intermediate 303, 1.72 mmol based on the boronic acid, 0.502 g), [1,1′-bis(diphenylphosphino)ferrocene]dichloropalladium (II) (0.22 mmol, 0.167 g) and sodium carbonate (1.14 mmol, 0.121 g) in acetonitrile/water (3:1) was degassed and heated to 90° C. for 1 h under an inert atmosphere. The solvent was concentrated in vacuo and the resultant crude mass was taken in EtOAc (50 mL), washed with water, brine, dried over Na₂SO₄, filtered and concentrated. The crude material was further purified by silica gel column chromatography (product eluted with 0.5% Et₃N in EtOAc) to yield 0.24 g of the product.

| | | Mass spectrum and ¹H | |
|---|---|---|---|
| Compound | Structure | NMR | SM |
| Example 796 | 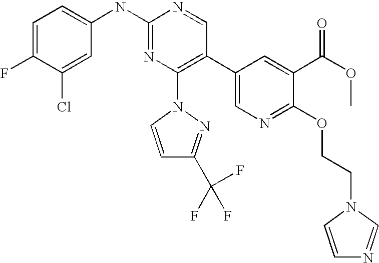 methyl 5-{2-[(3- chloro-4- fluorophenyl)amino]- 4-[3-(trifluoromethyl)- 1H-pyrazol-1- yl]pyrimidin-5-yl}-2- [2-(1H-imidazol-1- yl)ethoxy]pyridine-3- carboxylate | MS(ES): 603 (M + 1) for C₂₆H₁₉ClF₄N₈O₃. 400 MHz, DMSO-d₆: δ 3.79 (s, 3H), 4.40-4.41 (m, 2H), 4.57-4.58 (m, 2H), 6.89 (s, 1H), 7.05 (d, J = 2.48 Hz, 1H), 7.30 (s, 1H), 7.52 (t, J = 9.04 Hz, 1H), 7.71-7.74 (m, 2H), 7.90 (d, J = 2.36 Hz, 1H), 8.08 (dd, J = 2.48, 6.58 Hz, 1H), 8.27 (d, J = 2.40 Hz, 1H), 8.51 (s, 1H), 8.79 (s, 1H), 10.43 (s, 1H). | Intermediate 303 {6-[2-(1H- imidazol-1- yl)ethoxy]- 5- (methoxy- carbonyl) pyridin-3- yl}boronic acid |

### Example 797

### 5-{2-[(3-chloro-4-fluorophenyl)amino]-4-[3-(trifluoromethyl)-1H-pyrazol-1-yl]pyrimidin-5-yl}-2-[2-(1H-imidazol-1-yl)ethoxy]pyridine-3-carboxylic acid

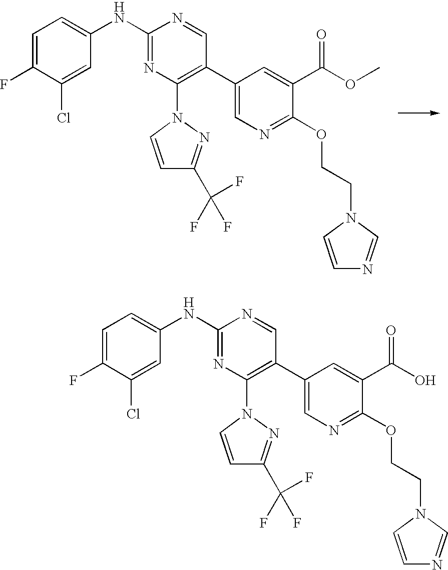

To 75 mg of methyl 5-{2-[(3-chloro-4-fluorophenyl)amino]-4-[3-(trifluoromethyl)-1H-pyrazol-1-yl]pyrimidin-5-yl}-2-[2-(1H-imidazol-1-yl)ethoxy]pyridine-3-carboxylate (Example 796, 0.12 mmol) taken in a mixture of dioxane (20 mL) and water (20 mL), was added Barium hydroxide monohydrate (0.48 mmol, 0.094 g) and warmed to 50° C. for 3 h. The mixture was then carefully acidified with 1 N HCl. It was extracted with ethyl acetate (50 mL) and the organic layer was washed with water and brine, dried over Na₂SO₄ and concentrated to yield 0.05 g of the title compound.

| | | Mass spectrum and ¹H | |
|---|---|---|---|
| Compound | Structure | NMR | SM |
| Example 797 | 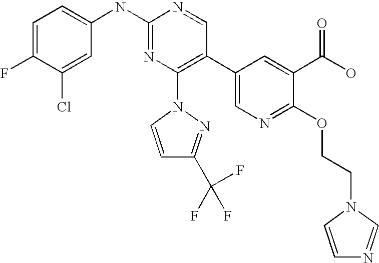 5-{2-[(3-chloro-4- fluorophenyl)amino]- 4-[3-(trifluoromethyl)- 1H-pyrazol-1- yl]pyrimidin-5-yl}-2- [2-(1H-imidazol-1- yl)ethoxy]pyridine-3- carboxylic acid | MS(ES): 589 (M + 1) for C₂₅H₁₇ClF₄N₈O₃. 400 MHz, DMSO-d₆: δ 4.38- 4.39 (m, 2H), 4.54 (s, 2H), 6.87 (s, 1H), 7.03 (s, 1H), 7.33 (s, 1H), 7.42 (t, J = 9.00 Hz, 1H), 7.70-7.72 (m, 2H), 7.86 (s, 1H), 8.08 (dd, J = 3.92 Hz, 1H), 8.21 (s, 1H), 8.48 (s, 1H), 8.78 (s, 1H), 10.40 (s, 1H). | Example 796 methyl 5- {2-[(3- chloro-4- fluorophenyl)- amino]-4- [3- (trifluoro- methyl)-1H- pyrazol-1- yl]pyrimidin- 5-yl}-2-[2- (1H- imidazol-1- yl)ethoxy) pyridine-3- carboxylate |

### Example 798

### methyl 5-{2-[(3-chloro-5-methylphenyl)amino]-4-[3-(trifluoromethyl)-1H-pyrazol-1-yl]pyrimidin-5-yl}-2-methoxypyridine-3-carboxylate

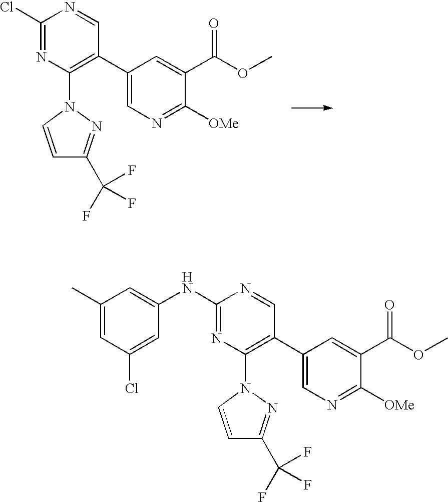

A suspension of methyl 5-{2-chloro-4-[3-(trifluoromethyl)-1H-pyrazol-1-yl]pyrimidin-5-yl}-2-methoxypyridine-3-carboxylate (Intermediate 325, 0.73 mmol, 0.30 g), 3-chloro-5-methylaniline (0.87 mmol, 0.12 g), tris(dibenzylideneacetone)dipalladium(0) (0.073 mmol, 0.07 g), 2-dicyclohexylphosphino-2′,4′,6′-triisopropyl-1,1′-biphenyl (0.15 mmol, 0.07 g) and sodium carbonate (0.73 mmol, 0.08 g) in acetonitrile/water (25 mL:6 mL) was heated to 90° C. for 30 min. The reaction mixture was concentrated. The residue was taken in ethyl acetate, washed with water and brine. The organic layer was separated, dried over sodium sulfate, filtered and concentrated. The crude mass was purified by silica gel column chromatography (60-120 mesh) using 25% ethyl acetate/hexanes as an eluent to yield 0.200 g of Example 798.

| | | Mass spectrum and | |
|---|---|---|---|
| Compound | Structure | ¹H NMR | SM |
| Example 798 | 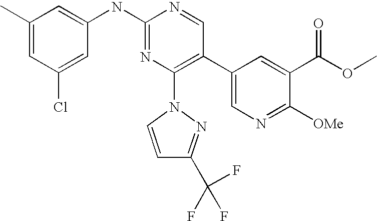 methyl 5-{2-[(3-chloro-5- methylphenyl)amino]-4-[3- (trifluoromethyl)-1H- pyrazol-1-yl]pyrimidin-5- yl}-2-methoxypyridine-3- carboxylate | MS(ES): 519 (M + 1) for C₂₃H₁₈ClF₃N₆O₃. 300 MHz, DMSO-d₆: δ 2.42 (s, 3H), 3.75 (s, 3H), 3.93 (s, 3H), 6.91 (s, 1H), 7.04 (d, J = 2.67 Hz, 1H), 7.62 (s, 1H), 7.79 (s, 1H), 7.85 (d, J = 2.46 Hz, 1H), 8.27 (d, J = 2.49 Hz, 1H), 8.46 (s, 1H), 8.80 (s, 1H), 10.34 (s, 1H). | Intermediate 325 methyl 5-{2- chloro-4-[3- (trifluoro- methyl)-1H- pyrazol-1- yl]pyrimidin- 5-yl}-2- methoxy- pyridine-3- carboxylate |

### Example 799

### 5-{2-[(3-chloro-5-methylphenyl)amino]-4-[3-(trifluoromethyl)-1H-pyrazol-1-yl]pyrimidin-5-yl}-2-methoxypyridine-3-carboxylic acid

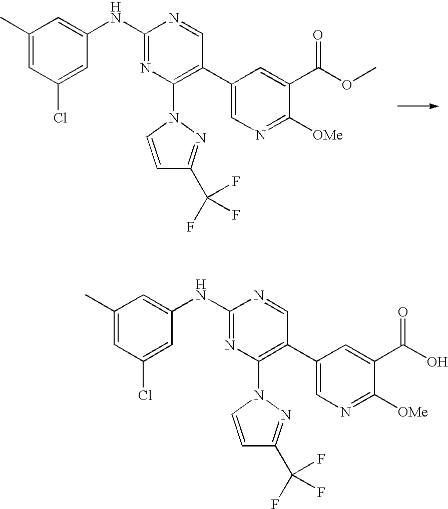

To 185 mg of methyl 5-{2-[(3-chloro-5-methylphenyl)amino]-4-[3-(trifluoromethyl)-1H-pyrazol-1-yl]pyrimidin-5-yl}-2-methoxypyridine-3-carboxylate (Example 798, 0.36 mmol) taken in a mixture of dioxane (5 mL) and water (5 mL), was added sodium hydroxide (0.9 mmol, 36 mg) and stirred at room temperature for 3 h. The reaction mixture was carefully acidified with 1 N HCl and further extracted with ethyl acetate (50 mL). The organic layer was washed with water and brine, dried over Na₂SO₄ and concentrated. The residue was dissolved in a minimum amount of CH₂Cl₂, then hexanes was added and the solid that precipitated was filtered, washed and dried in vacuo to yield 85 mg of the title compound.

| | | Mass spectrum and | |
|---|---|---|---|
| Compound | Structure | ¹H NMR | SM |
| Exaple 799 | 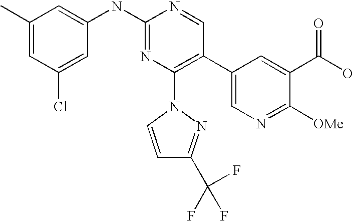 5-{2-[(3-Chloro-5- methylphenyl)amino]-4-[3- (trifluoromethyl)-1H- pyrazol-1-yl]pyrimidin-5- yl}-2-methoxypyridine-3- carboxylic acid | MS(ES): 505 (M + 1) for C₂₂H₁₆ClF₃N₆O₃. 400 MHz, DMSO-d₆: δ 2.31 (s, 3H), 3.92 (s, 3H), 6.92 (s, 1H), 7.03 (d, J = 2.56 Hz, 1H), 7.54 (s, 1H), 7.79 (s, 1H), 7.80 (m, 1H), 8.19 (s, 1H), 8.44 (s, 1H), 8.79 (s, 1H), 10.34 (s, 1H). | Example 798 methyl 5-{2- [(3-chloro-5- methylphenyl) amino]-4-[3- (trifluoro- methyl)-1H- pyrazol-1- yl]pyrimidin- 5-yl}-2- methoxy- pyridine-3- carboxylate |

### Example 800

### 5-{2-[(3-chloro-5-methylphenyl)amino]-4-[3-(trifluoromethyl)-1H-pyrazol-1-yl]pyrimidin-5-yl}-2-methoxy-N-(methylsulfonyl)pyridine-3-carboxamide

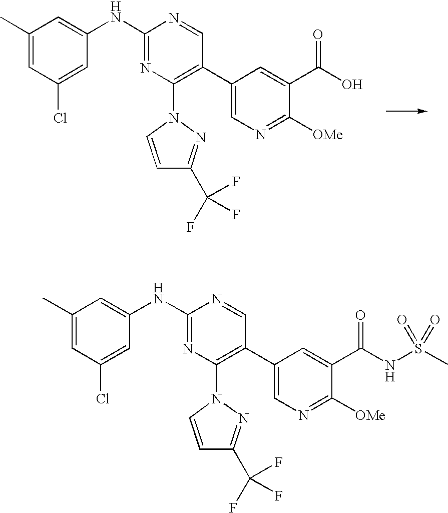

To a solution of 5-{2-[(3-chloro-5-methylphenyl)amino]-4-[3-(trifluoromethyl)-1H-pyrazol-1-yl]pyrimidin-5-yl}-2-methoxypyridine-3-carboxylic acid (Example 799, 0.089 mmol, 45 mg) in CH₂Cl₂ (5 mL), were added methanesulfonamide (0.22 mmol, 21 mg), 2-chloro-1-methylpyridinium iodide (0.11 mmol, 28 mg), 4-(Dimethylamino)pyridine (0.018 mmol, 2.1 mg) and triethylamine (0.27 mmol, 30 mg), and the solution stirred for 90 min at RT. The reaction mixture was diluted with dichloromethane and further washed with 25% citric acid solution, water and brine. The organic layer was dried over Na₂SO₄ and concentrated. The crude mass was purified by 60-120 mesh silica (product eluted with 1% MeOH in CHCl₃) to afford 30 mg of Example 800 as an off-white solid.

| | | Mass spectrum and | |
|---|---|---|---|
| Compound | Structure | ¹H NMR | SM |
| Example 800 | 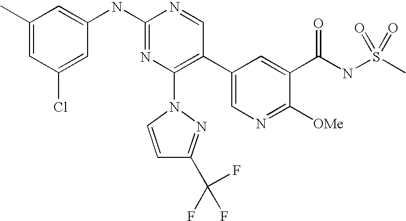 5-{2-[(3-chloro-5- methylphenyl)amino]-4-[3- (trifluoromethyl)-1H- pyrazol-1-yl]pyrimidin-5- yl}-2-methoxy-N- (methylsulfonyl)pyridine-3- carboxamide | MS(ES): 582 (M + 1) for C₂₃H₁₉ClF₃N₇O₄S. 400 MHz, DMSO-d₆: δ 2.32 (s, 3H), 3.33 (s, 3H), 3.97 (s, 3H), 6.94 (s, 1H), 7.06 (d, J = 2.60 Hz, 1H), 7.55 (s, 1H), 7.82 (s, 1H), 7.87 (d, J = 2.40 Hz, 1H), 8.19 (d, J= 2.40 Hz, 1H), 8.46 (d, J = 1.68 Hz, 1H), 8.81 (s, 1H), 10.39 (s, 1H), 11.75 (br s, 1H). | Example 799 5-{2-[(3- chloro-5- methylphenyl) amino]-4-[3- (trifluoro- methyl)-1H- pyrazol-1- yl]pyrimidin- 5-yl}-2- methoxy- pyridine-3- carboxylic acid |

### Example 801

### methyl 5-{2-[(3-chloro-5-methylphenyl)amino]-4-[5-methyl-3-(trifluoromethyl)-1H-pyrazol-1-yl]pyrimidin-5-yl}-2-methoxypyridine-3-carboxylate

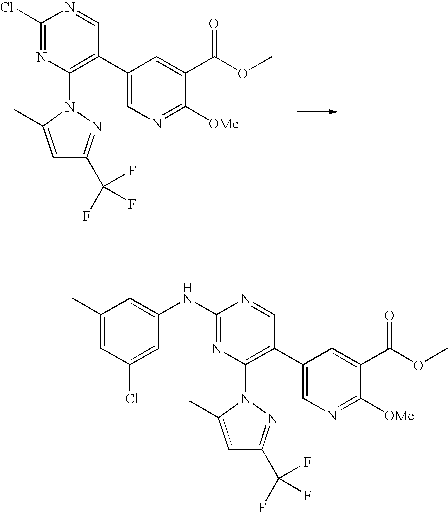

A suspension of methyl 5-{2-chloro-4-[5-methyl-3-(trifluoromethyl)-1H-pyrazol-1-yl]pyrimidin-5-yl}-2-methoxypyridine-3-carboxylate (Intermediate 327, 0.82 mmol, 0.35 g), 3-chloro-5-methylaniline (0.98 mmol, 0.14 g), tris(dibenzylideneacetone)dipalladium(0) (0.08 mmol, 0.075 g), 2-dicyclohexylphosphino-2′,4′,6′-triisopropyl-1,1′-biphenyl (0.16 mmol, 0.08 g) and sodium carbonate (0.8 mmol, 0.09 g) in acetonitrile/water (25 mL:6 mL) was heated to 90° C. for 30 min. The reaction mixture was concentrated. The residue was taken in ethyl acetate, washed with water and brine. The organic layer was separated, dried over sodium sulfate, filtered and concentrated. The crude mass was purified by silica gel column chromatography (60-120 mesh) using 20-25% ethyl acetate/hexanes as an eluent to yield 0.230 g of Example 801.

| | | Mass spectrum and | |
|---|---|---|---|
| Compound | Structure | ¹H NMR | SM |
| Example 801 | 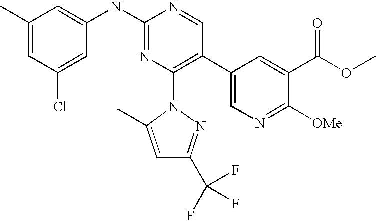 methyl 5-{2-[(3-chloro-5- methylphenyl)amino]-4-[5- methyl-3-(trifluoromethyl)- 1H-pyrazol-1-yl]pyrimidin- 5-yl}-2-methoxypyridine-3- carboxylate | MS(ES): 533 (M + 1) for C₂₄H₂₀ClF₃N₆O₃. 400 MHz, DMSO-d₆: δ 2.29 (s, 3H), 2.34 (s, 3H), 3.74 (s, 3H), 3.91 (s, 3H), 6.76 (s, 1H), 6.92 (s, 1H), 7.45 (s, 1H), 7.60 (d, J = 2.36 Hz, 1H), 7.79 (s, 1H), 8.22 (d, J = 2.40 Hz, 1H), 8.96 (s, 1H), 10.38 (s, 1H). | 3-chloro-5- methylaniline and Intermediate 327 methyl 5- {2-chloro-4- [5-methyl-3- (trifluoro- methyl)-1H- pyrazol-1- yl]pyrimidin- 5-yl}-2- methoxy- pyridine-3- carboxylate |

### Example 802

### 5-{2-[(3-chloro-5-methylphenyl)amino]-4-[5-methyl-3-(trifluoromethyl)-1H-pyrazol-1-yl]pyrimidin-5-yl}-2-methoxypyridine-3-carboxylic acid

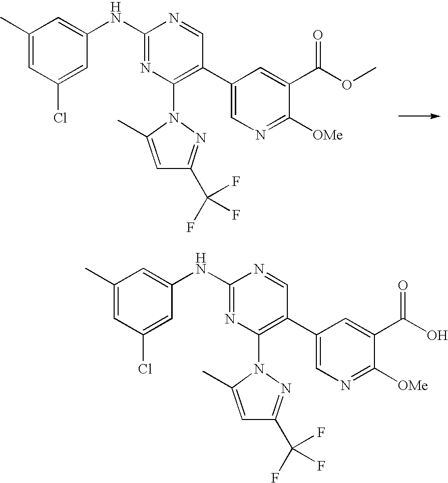

To 230 mg of methyl 5-12-[(3-chloro-5-methylphenyl)amino]-4-[5-methyl-3-(trifluoromethyl)-1H-pyrazol-1-yl]pyrimidin-5-yl}-2-methoxypyridine-3-carboxylate (Example 801, 0.43 mmol) taken in a mixture of dioxane (10 mL) and water (5 mL), was added 1 N aq.sodium hydroxide (1.08 mmol) and stirred overnight at room temperature. The reaction mixture was carefully acidified with 1 N HCl and further extracted with ethyl acetate (50 mL). The organic layer was washed with water and brine, dried over Na₂SO₄ and concentrated. The oily compound that was obtained was further stirred with hexanes and the solid that precipitated was filtered, and dried in vacuo to yield 150 mg of the title compound.

| | | Mass spectrum and | |
|---|---|---|---|
| Compound | Structure | ¹H NMR | SM |
| Example 802 | 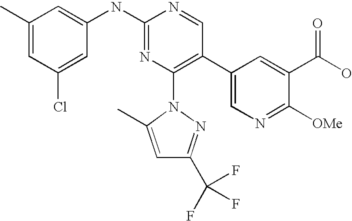 5-{2-[(3-chloro-5- methylphenyl)amino]-4-[5- methyl-3-(trifluoromethyl)- 1H-pyrazol-1-yl]pyrimidin- 5-yl}-2-methoxypyridine-3- carboxylic acid | MS(ES): 519 (M + 1) for C₂₃H₁₈ClF₃N₆O₃. 400 MHz, DMSO-d₆: δ 2.30 (s, 3H), 2.37 (s, 3H), 3.91 (s, 3H), 6.77 (s, 1H), 6.93 (s, 1H), 7.46 (s, 1H), 7.62 (d, J = 2.52 Hz, 1H), 7.80 (s, 1H), 8.17 (d, J = 2.48 Hz, 1H), 8.97 (s, 1H), 10.38 (s, 1H), 12.99 (br s, 1H). | Example 801 methyl 5-{2- [(3-chloro-5- methylphenyl) amino]-4-[5- methyl-3- (trifluoro- methyl)-1H- pyrazol-1- yl]pyrimidin- 5-yl}-2- methoxy- pyridine-3- carboxylate |

### Example 803

### 5-{2-[(3-chloro-5-methylphenyl)amino]-4-[5-methyl-3-(trifluoromethyl)-1H-pyrazol-1-yl]pyrimidin-5-yl}-2-methoxy-N-(methylsulfonyl)pyridine-3-carboxamide

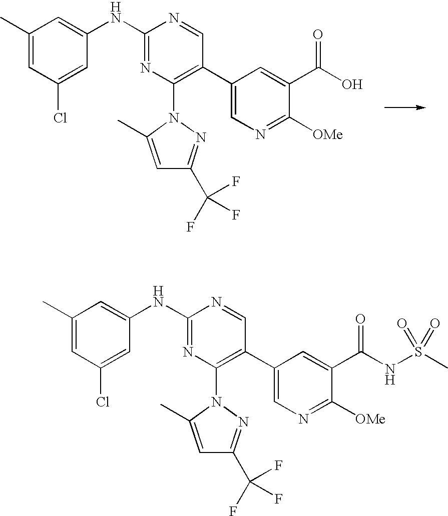

To a solution of 5-{2-[(3-chloro-5-methylphenyl)amino]-4-[5-methyl-3-(trifluoromethyl)-1H-pyrazol-1-yl]pyrimidin-5-yl}-2-methoxypyridine-3-carboxylic acid (Example 802, 0.22 mmol, 0.115 g) in CH₂Cl₂ (10 mL), were added methanesulfonamide (0.55 mmol, 52 mg), 2-chloro-1-methylpyridinium iodide (0.27 mmol, 70 mg), 4-(Dimethylamino)pyridine (0.044 mmol, 5.4 mg) and triethylamine (0.06 mmol, 0.1 mL), and the solution stirred for 90 min at RT. The reaction mixture was diluted with dichloromethane and further washed with 25% citric acid solution, water and brine. The organic layer was dried over Na₂SO₄ and concentrated. The crude mass was purified by 60-120 mesh silica (product eluted with 1% MeOH in CHCl₃) to afford 90 mg of Example 803.

| | | Mass spectrum and | |
|---|---|---|---|
| Compound | Structure | ¹H NMR | SM |
| Example 803 | 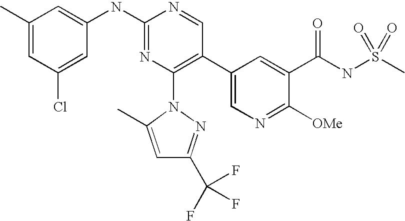 5-{2-[(3-chloro-5- methylphenyl)amino]-4-[5- methyl-3-(trifluoromethyl)- 1H-pyrazol-1-yl]pyrimidin- 5-yl}-2-methoxy-N- (methylsulfonyl)pyridine-3- carboxamide | MS(ES): 596 (M + 1) for C₂₄H₂₁ClF₃N₇O₄S. 400 MHz, DMSO-d₆: δ 2.30 (s, 3H), 2.36 (s, 3H), 3.33 (s, 3H), 3.93 (s, 3H), 6.79 (s, 1H), 6.94 (s, 1H), 7.47 (s, 1H), 7.70 (d, J = 2.40 Hz, 1H), 7.80 (s, 1H), 8.01 (d, J = 2.40 Hz, 1H), 8.98 (s, 1H), 10.41 (s, 1H), 11.78 (s, 1H). | Example 802 5-{2-[(3- chloro-5- methylphenyl) amino]-4-[5- methyl-3- (trifluoro- methyl)-1H- pyrazol-1- yl]pyrimidin- 5-yl}-2- methoxy- pyridine-3- carboxylic acid |

### Example 804

### 5-{2-[(3,5-dimethoxyphenyl)amino]-4-[5-methyl-3-(trifluoromethyl)-1H-pyrazol-1-yl]pyrimidin-5-yl}-2-methoxypyridine-3-carbohydrazide

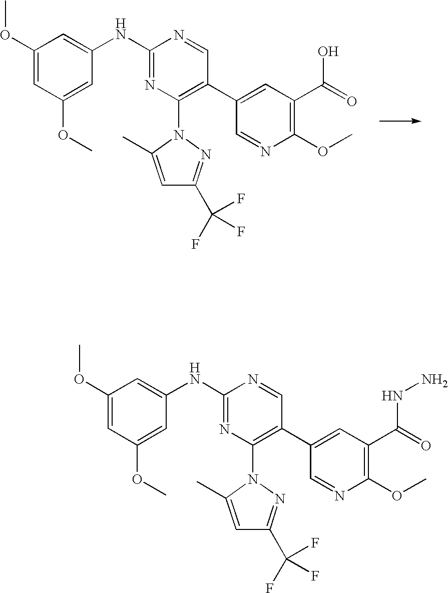

To a solution of 5-{2-[(3,5-dimethoxyphenyl)amino]-4-[5-methyl-3-(trifluoromethyl)-1H-pyrazol-1-yl]pyrimidin-5-yl}-2-methoxypyridine-3-carboxylic acid (Example 677, 0.37 mmol, 200 mg) and 4-(Dimethylamino)pyridine (0.075 mmol, 9 mg) in CH₂Cl₂ (25 mL) were added Hydrazine monohydrochloride (0.94 mmol, 64 mg), triethylamine (1.89 mmol, 0.255 mL), 2-chloro-1-methylpyridinium iodide (0.47 mmol, 120 mg) and stirred for 2 h at RT. The reaction mixture was diluted with dichloromethane (50 mL) and further washed with water (100 mL) and brine (75 mL). The organic layer was dried over Na₂SO₄ and concentrated. The crude mass was purified by 230-400 mesh silica (product eluted with 3-4% methanol/dichloromethane) to afford 100 mg of the title compound.

| | | Mass spectrum and ¹H | |
|---|---|---|---|
| Compound | Structure | NMR | SM |
| Example 804 | 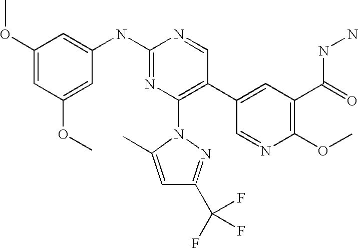 5-{2-[(3,5- dimethoxyphenyl)- amino]-4-[5-methyl-3- (trifluoromethyl)-1H- pyrazol-1- yl]pyrimidin-5-yl}-2- methoxypyridine-3- carbohydrazide | Taken to the next step based on LCMS without further purification. MS(ES): 545 (M + 1) for C₂₄H₂₃F₃N₈O₄. (70% pure by UPLC) | Example 677 5-{2-[(3,5- dimethoxy- phenyl)amino]- 4-[5- methyl-3- (trifluoro- methyl)-1H- pyrazol-1- yl]pyrimidin- 5-yl}-2- methoxy- pyridine-3- carboxylic acid |

### Example 805

### 5-(5-{2-[(3,5-dimethoxyphenyl)amino]-4-[5-methyl-3-(trifluoromethyl)-1H-pyrazol-1-yl]pyrimidin-5-yl}-2-methoxypyridin-3-yl)-1,3,4-oxadiazol-2(3H)-one

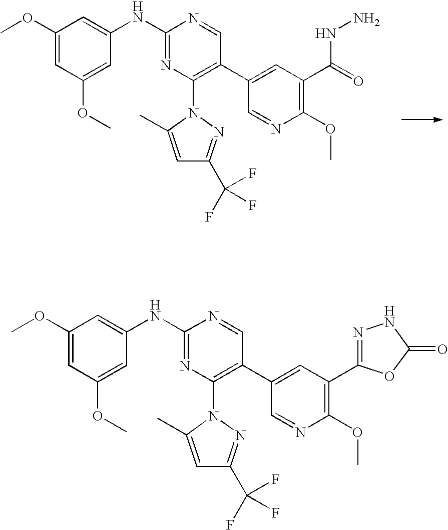

5-{2-[(3,5-dimethoxyphenyl)amino]-4-[5-methyl-3-(trifluoromethyl)-1H-pyrazol-1-yl]pyrimidin-5-yl}-2-methoxypyridine-3-carbohydrazide (Example 804, 0.18 mmol, 100 mg), 1,1′-carbonyldiimidazole (0.28 mmol, 44 mg), and N,N-diisopropylethylamine (0.28 mmol, 36 mg) in DMF (2 mL) were combined to give a white suspension. The reaction mixture was stirred at room temperature over 1 h, and then stirred at 50° C. for 1 h. The reaction mixture was poured onto ice-water, then extracted with ethyl acetate (50 mL×2) and further washed with water (75 mL) and brine (50 mL). The organic layer was dried over Na₂SO₄ and concentrated. The crude mass was purified by 230-400 mesh silica (product eluted with 1% methanol/dichloromethane) to afford Example 805 as off-white solid (46 mg).

| | | Mass spectrum and ¹H | |
|---|---|---|---|
| Compound | Structure | NMR | SM |
| Example 805 | 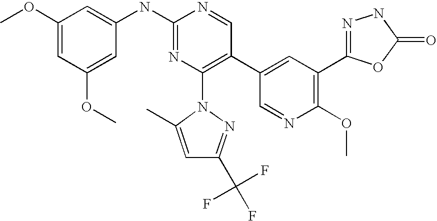 5-(5-{2-[(3,5- dimethoxyphenyl)- amino]-4-[5-methyl-3- (trifluoromethyl)-1H- pyrazol-1- yl]pyrimidin-5-yl}-2- methoxypyridin-3-yl)- 1,3,4-oxadiazol-2(3H)- one | MS(ES): 571 (M + 1) for C₂₅H₂₁F₃N₈O₅. 400 MHz, DMSO-d₆: δ 2.34 (s, 3H), 3.71 (s, 6H), 3.95 (s, 3H), 6.20 (t, J = 2.16 Hz, 1H), 6.74 (s, 1H), 7.03 (s, 1H), 7.04 (s, 1H), 7.61 (d, J = 2.40 Hz, 1H), 8.14(d, J = 2.36 Hz, 1H), 8.93 (s, 1H), 10.20 (s, 1H), 12.64 (s,1H). | Example 804 5-{2-[(3,5- dimethoxy- phenyl)amino]- 4-[5- methyl-3- (trifluoro- methyl)-1H- pyrazol-1- yl]pyrimidin- 5-yl}-2- methoxy- pyridine-3- carbo- hydrazide |

### Example 806

### 5-{2-[(3,5-dimethoxyphenyl)amino]-4-[3-(trifluoromethyl)-1H-pyrazol-1-yl]pyrimidin-5-yl}-2-methoxypyridine-3-carbohydrazide

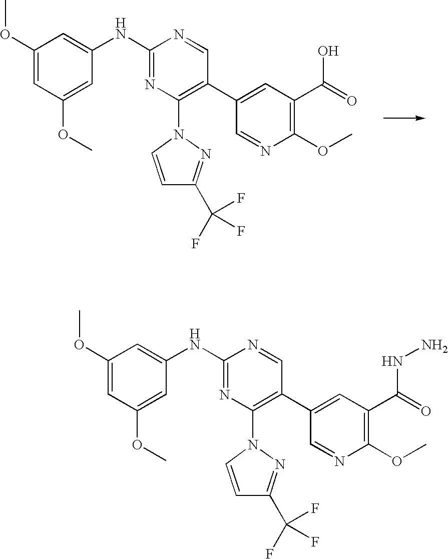

To a solution of 5-{2-[(3,5-dimethoxyphenyl)amino]-4-[3-(trifluoromethyl)-1H-pyrazol-1-yl]pyrimidin-5-yl}-2-methoxypyridine-3-carboxylic acid (Example 675, 0.38 mmol, 200 mg) and 4-(Dimethylamino)pyridine (0.077 mmol, 9 mg) in CH₂Cl₂ (25 mL) were added Hydrazine monohydrochloride (0.97 mmol, 66 mg), triethylamine (1.94 mmol, 2.62 mL), 2-chloro-1-methylpyridinium iodide (0.48 mmol, 124 mg) and stirred 8 h at RT. The reaction mixture was diluted with dichloromethane (50 mL) and further washed with water (100 mL) and brine (75 mL). The organic layer was dried over Na₂SO₄ and concentrated. The crude mass was purified by 230-400 mesh silica (product eluted with 3-4% methanol/dichloromethane) to afford 120 mg of Example 806.

| | | Mass spectrum and ¹H | |
|---|---|---|---|
| Compound | Structure | NMR | SM |
| Example 806 | 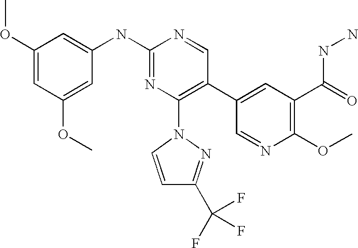 5-{2-[(3,5- dimethoxyphenyl)- amino]-4-[3- (trifluoromethyl)-1H- pyrazol-1- yl]pyrimidin-5-yl}-2- methoxypyridine-3- carbohydrazide | Taken to the next step based on LCMS without further purification. MS(ES): 531 (M + 1) for C₂₃H₂₁F₃N₈O₄. (40% pure by UPLC) | Example 675 5-{2-[(3,5- dimethoxy- phenyl)amino]- 4-[3- (trifluoro- methyl)-1H- pyrazol-1- yl]pyrimidin- 5-yl}-2- methoxy- pyridine-3- carboxylic acid |

### Example 807

### 5-(5-{2-[(3,5-dimethoxyphenyl)amino]-4-[3-(trifluoromethyl)-1H-pyrazol-1-yl]pyrimidin-5-yl}-2-methoxypyridin-3-yl)-1,3,4-oxadiazol-2(3H)-one

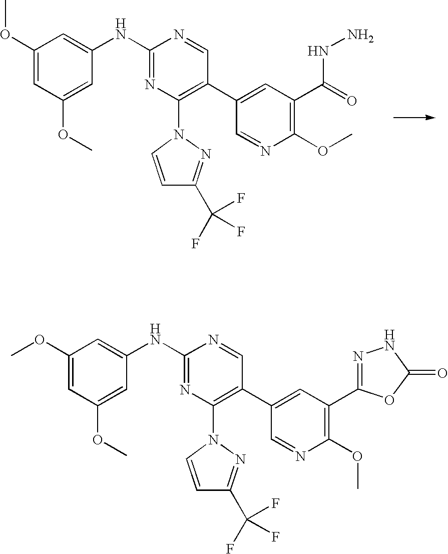

5-{2-[(3,5-dimethoxyphenyl)amino]-4-[3-(trifluoromethyl)-1H-pyrazol-1-yl]pyrimidin-5-yl}-2-methoxypyridine-3-carbohydrazide (Example 806, 0.36 mmol, 190 mg), 1,1′-carbonyldiimidazole (0.54 mmol, 87 mg), and N,N-diisopropylethylamine (0.54 mmol, 70 mg) in DMF (2 mL) were combined to give a white suspension. The reaction mixture was stirred at room temperature over 1 h, and then stirred at 50° C. for 1 h. The reaction mixture was poured on to ice-water, then extracted with ethyl acetate (50 mL×2) and further washed with water (75 mL) and brine (50 mL). The organic layer was dried over Na₂SO₄ and concentrated. The crude mass was purified by 230-400 mesh silica (product eluted with 1% methanol/dichloromethane) to afford Example 807 as light brown solid (33 mg).

| | | Mass spectrum and ¹H | |
|---|---|---|---|
| Compound | Structure | NMR | SM |
| Example 807 | 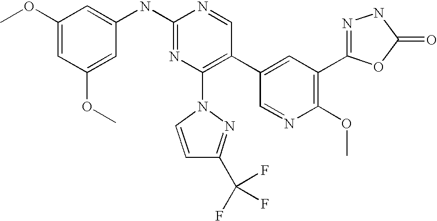 5-(5-{2-[(3,5- dimethoxyphenyl)- amino]-4-[3- (trifluoromethyl)-1H- pyrazol-1- yl]pyrimidin-5-yl}-2- methoxypyridin-3-yl)- 1,3,4-oxadiazol-2(3H)- one | MS(ES): 557 (M + 1) for C₂₄H₁₉F₃N₈O₅. 400 MHz, DMSO-d₆: δ 3.74 (s, 6H), 3.99 (s, 3H), 6.21 (t, J = 2.12 Hz, 1H), 7.04 (d, J = 2.52 Hz, 1H), 7.09 (s, 1H), 7.10 (s, 1H), 7.83 (d, J = 2.36 Hz, 1H), 8.25 (d, J= 2.36 Hz, 1H), 8.49 (s, 1H), 8.78 (s, 1H), 10.19 (s, 1H), 12.64 (s, 1H). | Example 806 5-{2-[(3,5- dimethoxy- phenyl)amino]- 4-[3- (trifluoro- methyl)-1H- pyrazol-1- yl]pyrimidin- 5-yl}-2- methoxy- pyridine-3- carbo- hydrazide |

### Example 808

### 5-{2-[(3,5-dimethoxyphenyl)amino]-4-[5-methyl-3-(trifluoromethyl)-1H-pyrazol-1-yl]pyrimidin-5-yl}-2-methoxy-N-methylpyridine-3-carboxamide

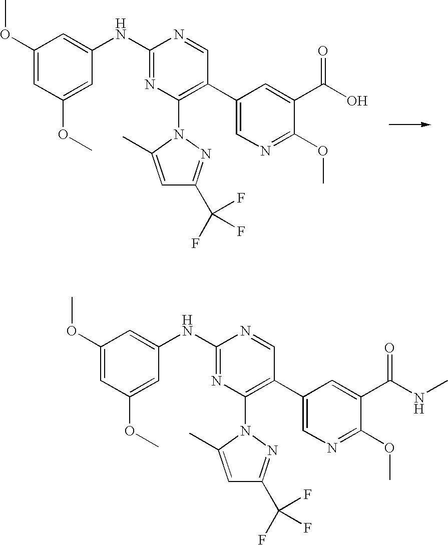

To a mixture of 5-{2-[(3,5-dimethoxyphenyl)amino]-4-[5-methyl-3-(trifluoromethyl)-1H-pyrazol-1-yl]pyrimidin-5-yl}-2-methoxypyridine-3-carboxylic acid (Example 677, 0.47 mmol, 250 mg), triethylamine (1.41 mmol, 0.2 mL, 0.143 mg) and methylamine hydrochloride (0.94 mmol, 64 mg) in dichloromethane was added T₃P (50% w/w solution in EtOAc; 0.94 mmol, 0.6 mL, 300 mg) at 0° C. The reaction mixture was slowly raised to room temperature and stirred 2 h. The mixture was then diluted with dichloromethane (15 mL), and the organic layer was successively washed with water (2×20 mL), 10% aq sodium bicarbonate solution (15 mL) and brine. The organic layer was dried over sodium sulphate and concentrated to yield 180 mg of Example 808.

| | | Mass spectrum and | |
|---|---|---|---|
| Compound | Structure | ¹H NMR | SM |
| Example 808 | 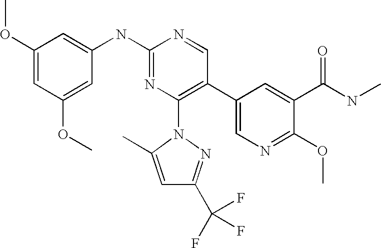 5-{2-[(3,5- dimethoxyphenyl)amino]-4- [5-methyl-3- (trifluoromethyl)-1H- pyrazol-1-yl]pyrimidin-5- yl}-2-methoxy-N- methylpyridine-3- carboxamide | MS(ES): 544 (M + 1) for C₂₅H₂₄F₃N₇O₄. 400 MHz, DMSO-d₆: δ 2.31 (s, 3H), 2.78 (d, J = 4.64 Hz, 3H), 3.73 (s, 6H), 3.96 (s, 3H), 6.21 (t, J = 2.16 Hz, 1H), 6.75 (s, 1H), 7.05 (d, J = 2.04 Hz, 2H), 7.78 (d, J = 2.48 Hz, 1H), 8.02 (d, J = 2.48 Hz, 1H), 8.21 (d, J = 4.72 Hz, 1H), 8.91 (s, 1H), 10.21 (s, 1H). | Example 677 5-{2-[(3,5- dimethoxy- phenyl)amino]-4- [5-methyl-3- (trifluoro- methyl)-1H- pyrazol-1- yl]pyrimidin- 5-yl}-2- methoxy- pyridine-3- carboxylic acid |

### Example 809

### 5-{2-[(3,5-dimethoxyphenyl)amino]-4-[3-(trifluoromethyl)-1H-pyrazol-1-yl]pyrimidin-5-yl}-2-methoxy-N-methylpyridine-3-carboxamide

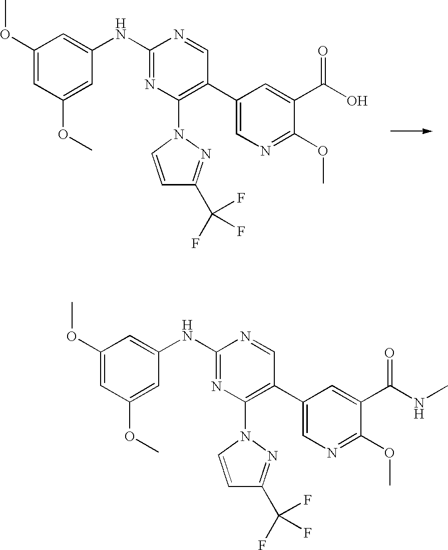

To a mixture of 5-{2-[(3,5-dimethoxyphenyl)amino]-4-[3-(trifluoromethyl)-1H-pyrazol-1-yl]pyrimidin-5-yl}-2-methoxypyridine-3-carboxylic acid (Example 675, 0.48 mmol, 250 mg), triethylamine (1.46 mmol, 0.20 mL, 145 mg) and methylamine hydrochloride (0.97 mmol, 66 mg) in dichloromethane (10 mL) was added T₃P (50% w/w solution in EtOAc, 0.97 mmol, 0.62 mL, 310 mg) slowly at 0° C. The reaction mixture was slowly raised to room temperature and stirred for 2 h. The reaction mixture was then diluted with dichloromethane (15 mL) and the dichloromethane layer was washed successively with water (2×20 mL), 10% aq sodium bicarbonate solution (15 mL) and brine. The organic layer was dried over sodium sulphate and concentrated. The crude material was purified by silica gel column chromatography (230-400 mesh) using 2% methanol/chloroform to yield 230 mg of Example 809.

| | | Mass spectrum and | |
|---|---|---|---|
| Compound | Structure | ¹H NMR | SM |
| Example 809 | 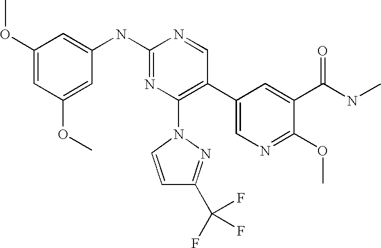 5-{2-[(3,5- dimethoxyphenyl)amino]-4- [3-(trifluoromethyl)-1H- pyrazol-1-yl]pyrimidin-5- yl}-2-methoxy-N- methylpyridine-3- carboxamide | MS(ES): 530 (M + 1) for C₂₄H₂₂F₃N₇O₄. 400 MHz, DMSO-d₆: δ 2.80 (d, J = 4.64 Hz, 3H), 3.75 (s, 6H), 4.00 (s, 3H), 6.21 (t, J = 2.16 Hz, 1H), 7.02 (d, J = 2.60 Hz, 1H), 7.11 (d, J = 2.16 Hz, 2H), 7.93 (d, J = 2.48 Hz, 1H), 8.16 (d, J = 2.48 Hz, 1H), 8.24 (d, J = 4.68 Hz, 1H), 8.45 (d, J = 1.64 Hz, 1H), 8.76 (s, 1H), 10.18 (s, 1H). | Example 675 5-{2-[(3,5- dimethoxy- phenyl)amino]-4- [3-(trifluoro- methyl)-1H- pyrazol-1- yl]pyrimidin- 5-yl}-2- methoxy- pyridine- 3-carboxylic acid |

### Example 810

### 5-{2-[(3,5-dimethoxyphenyl)amino]-4-[5-methyl-3-(trifluoromethyl)-1H-pyrazol-1-yl]pyrimidin-5-yl}-N,2-dimethoxypyridine-3-carboxamide

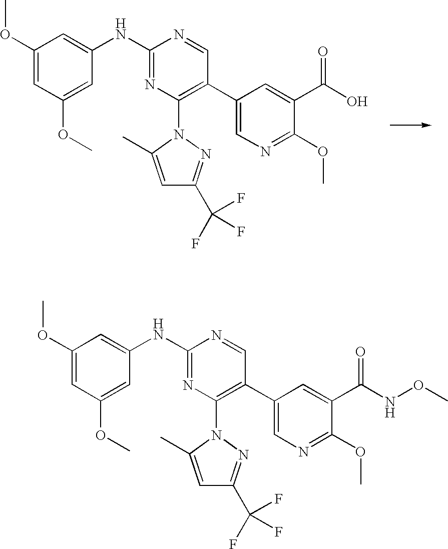

To a mixture of 5-{2-[(3,5-dimethoxyphenyl)amino]-4-[5-methyl-3-(trifluoromethyl)-1H-pyrazol-1-yl]pyrimidin-5-yl}-2-methoxypyridine-3-carboxylic acid (Example 677, 0.47 mmol, 250 mg), triethylamine (0.94 mmol, 0.13 mL, 94 mg) and methoxylamine hydrochloride (0.7 mmol, 59 mg) in dichloromethane, was added TBTU (0.56 mmol, 182 mg) slowly at 0° C. The reaction mixture was slowly raised to room temperature and stirred for 2 h. The reaction mixture was then diluted with dichloromethane (15 mL) and the dichloromethane solution was washed successively with water (2×20 mL), 10% aq sodium bicarbonate solution (20 mL) and brine. The organic layer was dried over sodium sulphate, and concentrated to yield 170 mg of Example 810.

| | | Mass spectrum and | |
|---|---|---|---|
| Compound | Structure | ¹H NMR | SM |
| Example 810 | 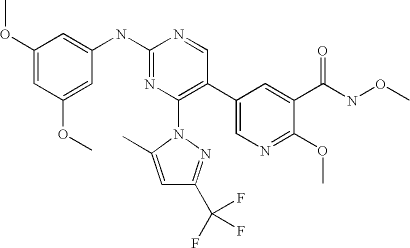 5-{2-[(3,5- dimethoxyphenyl)amino]-4- [5-methyl-3- (trifluoromethyl)-1H- pyrazol-1-yl]pyrimidin-5- yl}-N,2-dimethoxypyridine- 3-carboxamide | MS(ES): 560 (M + 1) for C₂₅H₂₄F₃N₇O₅. 400 MHz, DMSO-d₆: δ 2.31 (s, 3H), 3.68 (s, 3H), 3.73 (s, 6H), 3.93 (s, 3H), 6.22 (t, J = 2.00 Hz, 1H), 6.76 (s, 1H), 7.05 (d, J = 1.96 Hz, 2H), 7.65 (d, J = 2.36 Hz, 1H), 8.03 (d, J = 2.36 Hz, 1H), 8.93 (s, 1H), 10.22 (s, 1H), 11.31 (s, 1H). | Example 677 5-{2-[(3,5- dimethoxy- phenyl)amino]-4- [5-methyl-3- (trifluoro- methyl)-1H- pyrazol-1-yl ]pyrimidin-5-yl}-2- methoxy- pyridine-3- carboxylic acid |

### Example 811

### 5-{2-[(3,5-dimethoxyphenyl)amino]-4-[3-(trifluoromethyl)-1H-pyrazol-1-yl]pyrimidin-5-yl}-N,2-dimethoxypyridine-3-carboxamide

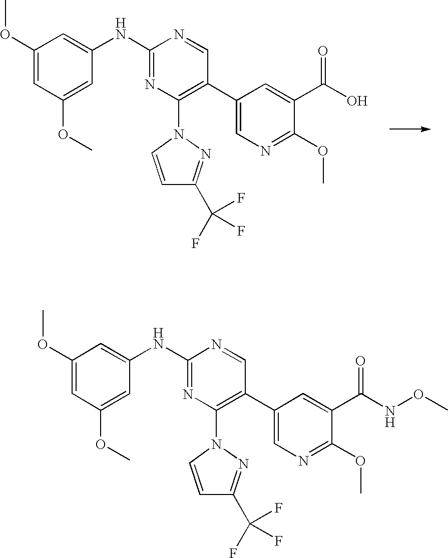

To a mixture of 5-{2-[(3,5-dimethoxyphenyl)amino]-4-[3-(trifluoromethyl)-1H-pyrazol-1-yl]pyrimidin-5-yl}-2-methoxypyridine-3-carboxylic acid (Example 675, 0.48 mmol, 250 mg), triethylamine (0.97 mmol, 0.14 mL, 98 mg) and methoxylamine hydrochloride (0.73 mmol, 61 mg) in dichloromethane, was added TBTU (0.58 mmol, 187 mg) slowly at 0° C. The reaction mixture was slowly raised to room temperature and stirred for 2 h. The reaction mixture was then diluted with dichloromethane (15 mL) and the dichloromethane solution was washed successively with water (2×20 mL), 10% aq sodium bicarbonate solution (20 mL) and brine. The organic layer was dried over sodium sulphate, and concentrated. The crude material was purified by silica gel column chromatography using 2% methanol/chloroform to yield 220 mg of Example 811.

| | | Mass spectrum and | |
|---|---|---|---|
| Compound | Structure | ¹H NMR | SM |
| Example 811 | 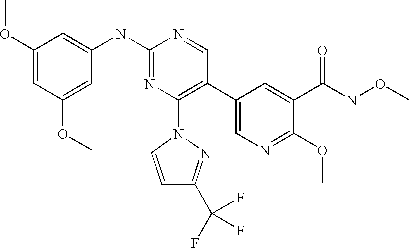 5-{2-[(3,5- dimethoxyphenyl)amino]-4- [3-(trifluoromethyl)-1H- pyrazol-1-yl]pyrimidin-5- yl}-N,2-dimethoxypyridine- 3-carboxamide | MS(ES): 546 (M + 1) for C₂₄H₂₂F₃N₇O₅. 400 MHz, DMSO-d₆: δ 3.69 (s, 3H), 3.75 (s, 6H), 3.96 (s, 3H), 6.21 (t, J = 2.16 Hz, 1H), 7.03 (d, J = 2.64 Hz, 1H), 7.11 (d, J = 2.16 Hz, 2H), 7.85 (d, J = 2.36 Hz, 1H), 8.16 (d, J = 2.44 Hz, 1H), 8.45 (d, J = 1.56 Hz, 1H), 8.76 (s, 1H), 10.18 (s, 1H), 11.31 (s, 1H). | Example 675 5-{2-[(3,5- dimethoxy- phenyl)amino]-4- [3- (trifluoro- methyl)-1H- pyrazol-1- yl]pyrimidin- 5-yl}-2- methoxy- pyridine-3- carboxylic acid |

### Example 812

### [5-{2-[(3,5-dimethoxyphenyl)amino]-4-[5-methyl-3-(trifluoromethyl)-1H-pyrazol-1-yl]pyrimidin-5-yl}-2-methoxy-N-[2-(methylsulfonyl)ethyl]pyridine-3-carboxamide

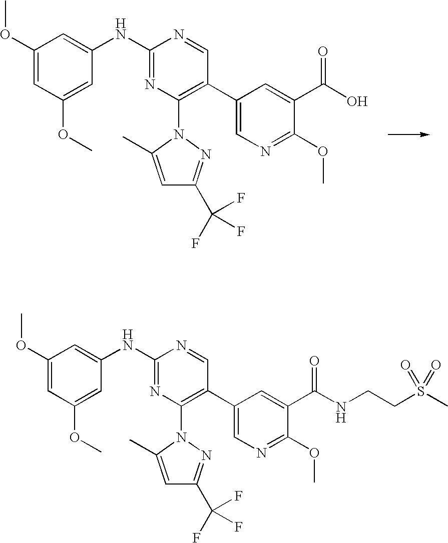

To a solution of 5-{2-[(3,5-dimethoxyphenyl)amino]-4-[5-methyl-3-(trifluoromethyl)-1H-pyrazol-1-yl]pyrimidin-5-yl}-2-methoxypyridine-3-carboxylic acid (Example 677, 0.28 mmol, 150 mg) and [2-(methylsulfonyl)ethyl]amine (0.71 mmol, 87 mg) in CH₂Cl₂ (5 mL), were add 2-chloro-1-methylpyridinium iodide (0.35 mmol, 90 mg), 4-(Dimethylamino)pyridine (0.056 mmol, 7 mg) and triethylamine (0.85 mmol, 85 mg), and the solution stirred for 90 min at RT. The reaction mixture was diluted with dichloromethane and further washed with 25% citric acid solution, water and brine. The organic layer was dried over Na₂SO₄ and concentrated. The crude mass was purified by 60-120 mesh silica (product eluted with 1% MeOH in CHCl₃) to afford 80 mg of Example 812 as an off-white solid.

| | | Mass spectrum and | |
|---|---|---|---|
| Compound | Structure | ¹H NMR | SM |
| Example 812 | 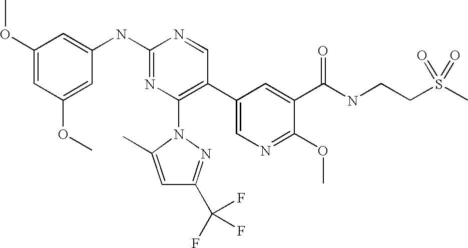 5-{2-[(3,5- dimethoxyphenyl)amino]-4- [5-methyl-3- (trifluoromethyl)-1H- pyrazol-1-yl]pyrimidin-5- yl}-2-methoxy-N-[2- (methylsulfonyl)ethyl]- pyridine-3-carboxamide | MS(ES): 636 (M + 1) for C₂₇H₂₈F₃N₇O₆S. 400 MHz, DMSO-d₆: δ 2.31 (s, 3H), 3.04 (s, 3H), 3.35 (t, J = 6.40 Hz, 2H), 3.69 (m, 2H, partly merges with water peak), 3.71 (s, 6H), 3.95 (s, 3H),6.20 (d, J = 1.20 Hz, 1H), 6.73 (s, 1H), 7.04 (s, 2H), 7.81 (dd, J = 1.20, 2.48 Hz, 1H), 8.05 (dd, J = 1.16, 2.42 Hz, 1H), 8.59 (t, J = 5.84 Hz, 1H), 8.89 (d, J = 1.16 Hz, 1H), 10.19 (s, 1H). | Example 677 5-{2-[(3,5- dimethoxy- phenyl)amino]-4- [5-methyl-3- (trifluoro- methyl)-1H- pyrazol-1- yl]pyrimidin- 5-yl}-2- methoxy- pyridine-3- carboxylic acid |

### Example 813

### 5-{2-[(3,5-dimethoxyphenyl)amino]-4-[3-(trifluoromethyl)-1H-pyrazol-1-yl]pyrimidin-5-yl}-2-methoxy-N-[2-(methylsulfonyl)ethyl]pyridine-3-carboxamide

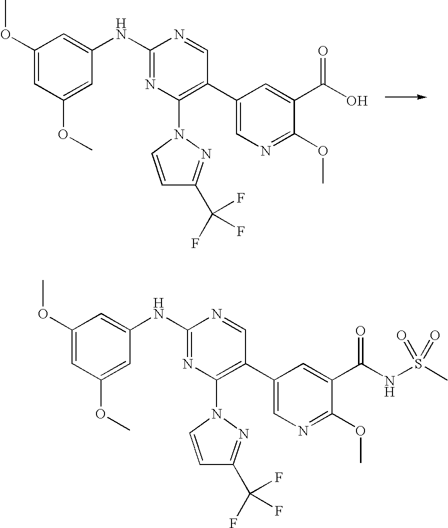

To a solution of 5-{2-[(3,5-dimethoxyphenyl)amino]-4-[3-(trifluoromethyl)-1H-pyrazol-1-yl]pyrimidin-5-yl}-2-methoxypyridine-3-carboxylic acid (Example 675, 0.29 mmol, 150 mg) and [2-(methylsulfonyl)ethyl]amine (0.72 mmol, 89 mg)in CH₂Cl₂ (5 mL), were added 2-chloro-1-methylpyridinium iodide (0.36 mmol, 92 mg), 4-(Dimethylamino)pyridine (0.058 mmol, 7 mg) and triethylamine (0.87 mmol, 88 mg), and the solution stirred for 90 min at RT. The reaction mixture was diluted with dichloromethane and further washed with 25% citric acid solution, water and brine. The organic layer was dried over Na₂SO₄ and concentrated. The crude mass was purified by 60-120 mesh silica (product eluted with 1% MeOH in CHCl₃) to afford 95 mg of Example 813 as an off-white solid.

| | | Mass spectrum and | |
|---|---|---|---|
| Compound | Structure | ¹H NMR | SM |
| Example 813 | 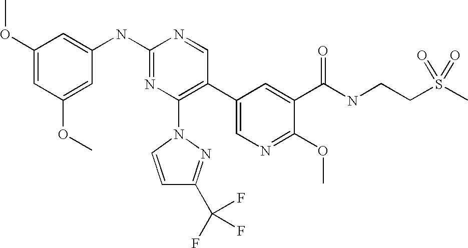 5-{2-[(3,5- dimethoxyphenyl)amino]-4- [3-(trifluoromethyl)-1H- pyrazol-1-yl]pyrimidin-5- yl}-2-methoxy-N-[2- (methylsulfonyl)ethyl]- pyridine-3-carboxamide | MS(ES): 622 (M + 1) for C₂₆H₂₆F₃N₇O₆S. 400 MHz, DMSO-d₆: δ 3.04 (s, 3H), 3.36 (t, J = 6.76 Hz, 2H), 3.71 (m, 2H), 3.73 (s, 6H), 3.99 (s, 3H), 6.20 (t, J = 2.20 Hz, 1H), 7.02 (d, J = 2.60 Hz, 1H), 7.10 (d, J = 2.20 Hz, 2H), 7.97 (d, J = 2.52 Hz, 1H), 8.18 (d, J = 2.48 Hz, 1H), 8.45 (d, J = 1.68 Hz,1H), 8.64 (s, 1H), 8.75 (s,1H), 10.18 (s, 1H). | Example 675 5-{2-[(3,5- dimethoxy- phenyl)amino]-4- [3-(trifluoro- methyl)-1H- pyrazol-1- yl]pyrimidin- 5-yl}-2- methoxy- pyridine-3- carboxylic acid |

### Example 814

### 5-{2-[(3,5-dimethoxyphenyl)amino]-4-[3-(trifluoromethyl)-1H-pyrazol-1-yl]pyrimidin-5-yl}-N-(ethylsulfonyl)-2-methoxypyridine-3-carboxamide

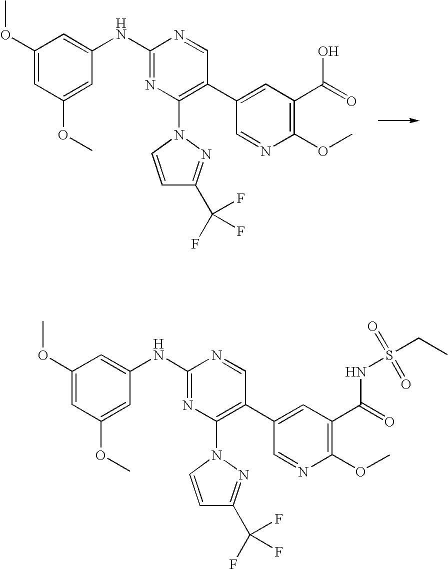

To a solution of 5-{2-[(3,5-dimethoxylphenyl)amino]-4-[3-(trifluoromethyl)-1H-pyrazol-1-yl]pyrimidin-5-yl}-2-methoxypyridine-3-carboxylic acid Example 675 (0.48 mmol, 0.250 g) in CH₂Cl₂ (25 mL), were added ethanesulfonamide (Intermediate 328, 1.16 mmol, 0.126 g), triethylamine (1.45 mmol, 0.204 mL), 2-chloro-1-methylpyridinium iodide (0.58 mmol, 0.148 g) and 4-(Dimethylamino)pyridine (0.097 mmol, 0.012 g) and stirred overnight at RT. The reaction mixture was diluted with dichloromethane (50 mL) and further washed with 25% citric acid solution (2×50 mL), water (100 mL) and brine (75 mL). The organic layer was dried over Na₂SO₄ and concentrated. The crude mass was purified by 60-120 mesh silica (product eluted with 1-2% MeOH in CHCl₃) to afford 120 mg of white solid of Example 814.

| | | Mass spectrum and ¹H | |
|---|---|---|---|
| Compound | Structure | NMR | SM |
| Example 814 | 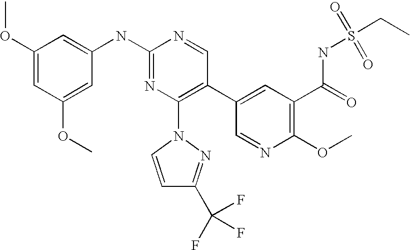 5-{2-[(3,5- dimethoxyphenyl)- amino]-4-[3- (trifluoromethyl)-1H- pyrazol-1- yl]pyrimidin-5-yl}-N- (ethylsulfonyl)-2- methoxypyridine-3- carboxamide | MS(ES): 608 (M + 1) for C₂₅H₂₄F₃N₇O₆S. 400 MHz, DMSO-d₆: δ 1.26 (t, J = 7.32 Hz, 3H), 3.46 (q, J = 7.32 Hz, 2H), 3.73 (s, 6H), 3.95 (s, 3H), 6.20 (t, J = 2.08 Hz, 1H), 7.04 (d, J = 2.56 Hz, 1H), 7.10 (d,J = 2.08 Hz, 2H), 7.80 (d, J = 2.36 Hz, 1H), 8.19 (d, J = 2.36 Hz, 1H), 8.44 (d, J = 1.76 Hz, 1H), 8.77 (s, 1H), 10.19 (s, 1H), 11.70 (s, 1H). | Intermediate 328 ethanesul- fonamide and Example 675 5-{2-[(3,5- dimethoxy- phenyl)amino]- 4-[3-(trifluoro- methyl)-1H- pyrazol-1- yl]pyrimidin- 5-yl}-2-methoxy- pyridine-3- carboxylic acid |

### Example 815

### 5-{2-[(3,5-dimethoxyphenyl)amino]-4-[5-methyl-3-(trifluoromethyl)-1H-pyrazol-1-yl]pyrimidin-5-yl}-N-(ethylsulfonyl)-2-methoxypyridine-3-carboxamide

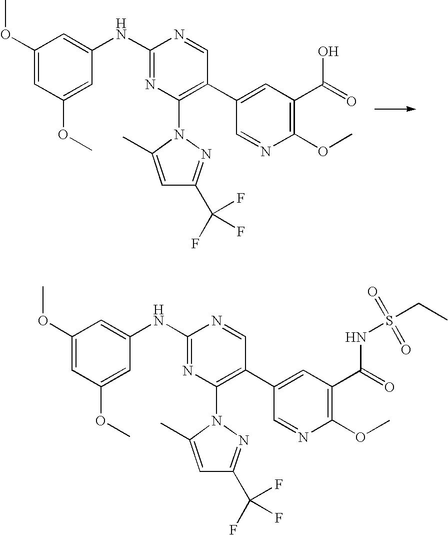

To a solution of 5-{2-[(3,5-dimethoxyphenyl)amino]-4-[5-methyl-3-(trifluoromethyl)-1H-pyrazol-1-yl]pyrimidin-5-yl}-2-methoxypyridine-3-carboxylic acid (Example 677, 0.19 mmol, 100 mg) in CH₂Cl₂ (10 mL), were added ethanesulfonamide (Intermediate 328, 0.47 mmol, 52 mg), triethylamine (0.56 mmol, 57 mg), 2-chloro-1-methylpyridinium iodide (0.23 mmol, 60 mg) and 4-(Dimethylamino)pyridine (0.037 mmol, 4.5 mg) and stirred at RT for 3-4 h. The reaction mixture was diluted with dichloromethane and further washed with water and brine. The organic layer was dried over Na₂SO₄ and concentrated. The crude mass was purified by 60-120 mesh silica (product eluted with 1% MeOH in CHCl₃) to afford 40 mg of the title compound.

| | | Mass spectrum and ¹H | |
|---|---|---|---|
| Compound | Structure | NMR | SM |
| Example 815 | 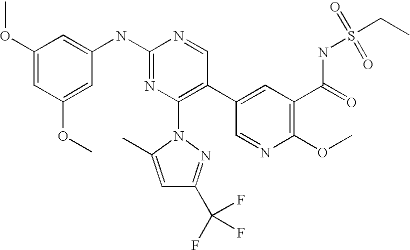 5-{2-[(3,5- dimethoxyphenyl)- amino]-4-[5-methyl-3- (trifluoromethyl)-1H- pyrazol-1- yl]pyrimidin-5-yl}-N- (ethylsulfonyl)-2- methoxypyridine-3- carboxamide | MS(ES): 622 (M + 1) for C₂₆H₂₆F₃N₇O₆S. 400 MHz, DMSO-d₆: δ 1.25 (t, J = 7.36 Hz, 3H), 2.30 (s, 3H), 3.44 (q, J = 7.36 Hz, 2H), 3.72 (s, 6H), 3.92 (s, 3H), 6.21 (t, J = 2.00 Hz, 1H), 6.76 (s, 1H), 7.05 (d, J = 1.92 Hz, 2H), 7.62 (d, J = 2.40 Hz, 1H), 8.02 (d, J = 2.28 Hz, 1H), 8.94 (s, 1H), 10.22 (s, 1H), 11.72 (s, 1H). | Intermediate 328 ethanesul- fonamide and Example 677 5-{2-[(3,5- dimethoxy- phenyl)amino]- 4-[5-methyl-3- (trifluoro- methyl)-1H- pyrazol-1- yl]pyrimidin- 5-yl}-2-methoxy- pyridine-3- carboxylic acid |

### Example 816

### 5-{2-[(3,5-dimethoxyphenyl)amino]-4-[3-(trifluoromethyl)-1H-pyrazol-1-yl]pyrimidin-5-yl}-2-methoxy-N-(propylsulfonyl)pyridine-3-carboxamide

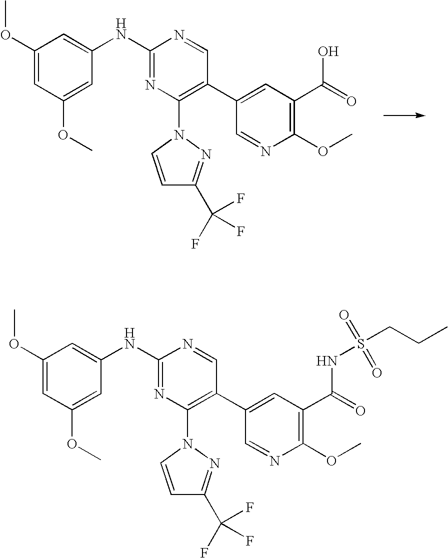

To a solution of 5-{2-[(3,5-dimethoxylphenyl)amino]-4-[3-(trifluoromethyl)-1H-pyrazol-1-yl]pyrimidin-5-yl}-2-methoxypyridine-3-carboxylic acid (0.48 mmol, 0.250 g) in CH₂Cl₂ (25 mL), were added propane-1-sulfonamide (Intermediate 329, 1.16 mmol, 0.143 g), triethylamine (1.45 mmol, 0.204 mL), 2-chloro-1-methylpyridinium iodide (0.58 mmol, 0.148 g) and 4-(Dimethylamino)pyridine (0.097 mmol, 0.012 g) and stirred overnight at RT. The reaction mixture was diluted with dichloromethane (50 mL) and further washed with 25% citric acid solution (2×50 mL), water (100 mL) and brine (75 mL). The organic layer was dried over Na₂SO₄ and concentrated. The crude mass was purified by 60-120 mesh silica (product eluted with 1.5-2.5% MeOH in CHCl₃) to afford 170 mg of Example 816 as a white solid.

| | | Mass spectrum and ¹H | |
|---|---|---|---|
| Compound | Structure | NMR | SM |
| Example 816 | 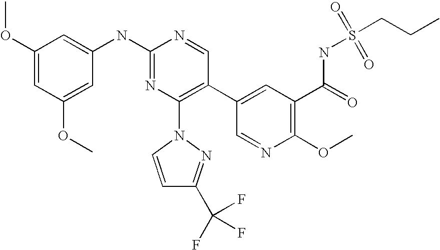 5-{2-[(3,5-dimethoxyphenyl)amino]-4-[3-(trifluoromethyl)-1H- pyrazol-1-yl]pyrimidin-5-yl}-2-methoxy-N- (propylsulfonyl)pyridine-3-carboxamide | MS(ES): 622 (M + 1) for C₂₆H₂₆F₃N₇O₆S. 400 MHz, DMSO-d₆: δ 1.00 (t, J = 7.44 Hz, 3H), 1.73 (q, J = 7.52 Hz, 2H), 3.44 (t, J = 7.64 Hz, 2H), 3.73 (s, 6H), 3.95 (s, 3H), 6.20 (t, J = 2.00 Hz, 1H), 7.04 (d, J = 2.56 Hz, 1H), 7.10 (d, J = 2.04 Hz, 2H), 7.80 (d, J = 2.28 Hz, 1H), 8.19 (d, J = 2.36 Hz, 1H), 8.44 (d, J = 1.88 Hz, 1H), 8.77 (s, 1H), 10.19 (s, 1H), 11.71 (s, 1H). | Intermediate 329 propane-1- sulfonamide and Example 675 5-{2-[(3,5- dimethoxy- phenyl) amino]-4-[3- (trifluoromethyl)- 1H-pyrazol-1- yl]pyrimidin- 5-yl}-2- methoxypyridine-3- carboxylic acid |

### Example 817

### 5-{2-[(3,5-dimethoxyphenyl)amino]-4-[5-methyl-3-(trifluoromethyl)-1H-pyrazol-1-yl]pyrimidin-5-yl}-2-methoxy-N-(propylsulfonyl)pyridine-3-carboxamide

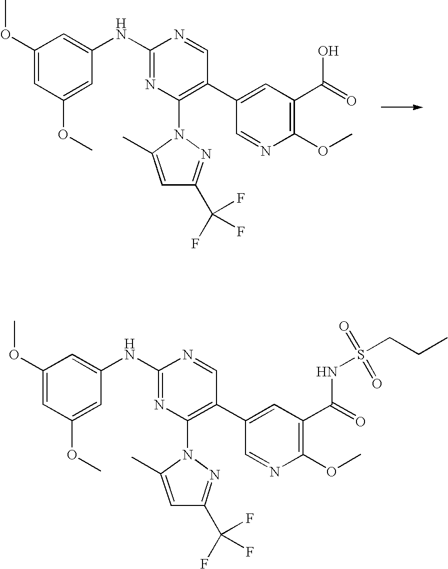

To a solution of 5-{2-[(3,5-dimethoxyphenyl)amino] -4-[5-methyl-3-(trifluoromethyl)-1H-pyrazol-1-yl]pyrimidin-5-yl}-2-methoxypyridine-3-carboxylic acid (Example 677, 0.19 mmol, 100 mg) in CH₂Cl₂ (10 mL), were added propane-1-sulfonamide (Intermediate 329, 0.47 mmol, 60 mg), triethylamine (0.56 mmol, 57 mg), 2-chloro-1-methylpyridinium iodide (0.23 mmol, 60 mg) and 4-(Dimethylamino)pyridine (0.037 mmol, 4.5 mg) and stirred at RT for 1 h. The reaction mixture was diluted with dichloromethane and further washed with water and brine. The organic layer was dried over Na₂SO₄ and concentrated. The crude mass was purified by 60-120 mesh silica (product eluted with 1% MeOH in CHCl₃) to afford 40 mg of the title compound.

| | | Mass spectrum and ¹H | |
|---|---|---|---|
| Compound | Structure | NMR | SM |
| Example 817 | 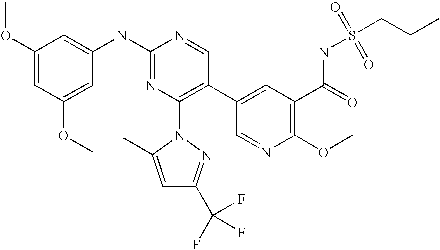 5-{2-[(3,5-dimethoxyphenyl)amino]-4-[5-methyl-3-(trifluoromethyl)-1H-pyrazol-1-yl]pyrimidin-5-yl}-2-methoxy-N-(propylsulfonyl) pyridine-3-carboxamide | MS(ES): 636 (M + 1) for C₂₇H₂₈F₃N₇O₆S. 400 MHz, DMSO-d₆: δ 1.01 (t, J = 7.44 Hz, 3H), 1.71- 1.76 (m, 2H), 2.32 (s, 3H), 3.43 (t, J = 7.96 Hz, 2H), 3.73 (s, 6H), 3.93 (s, 3H), 6.22 (t, J = 2.16 Hz, 1H), 6.77 (s, 1H), 7.06 (d, J = 2.12 Hz, 2H), 7.63 (d, J = 2.44 Hz, 1H), 8.03 (d, J = 2.44 Hz, 1H), 8.94 (s, 1H), 10.23 (s, 1H), 11.75 (s, 1H). | Intermediate 329 propane-1- sulfonamide and Example 677 5-{2-[(3,5- dimethoxyphenyl) amino]-4-[5- methyl-3- (trifluoromethyl)-1H- pyrazol-1- yl]pyrimidin- 5-yl}-2- methoxy pyridine-3- carboxylic acid |

### Example 818

### 5-{2-[(3,5-dimethoxyphenyl)amino]-4-[3-(trifluoromethyl)-1H-pyrazol-1-yl]pyrimidin-5-yl}-2-methoxy-N-(propan-2-ylsulfonyl)pyridine-3-carboxamide

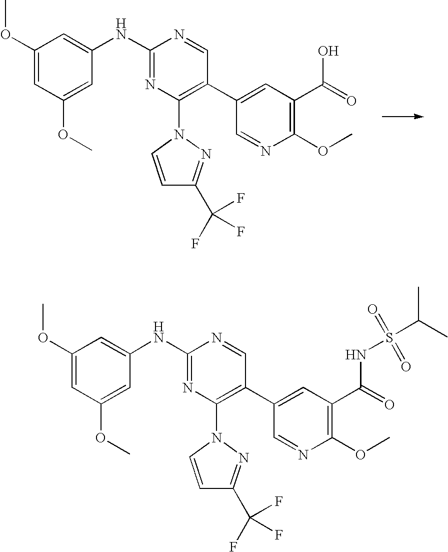

To a solution of 5-{2-[(3,5-dimethoxylphenyl)amino]-4-[3-(trifluoromethyl)-1H-pyrazol-1-yl]pyrimidin-5-yl}-2-methoxypyridine-3-carboxylic acid (Example 675, 0.38 mmol, 0.2 g) in CH₂Cl₂ (10 mL), were added propane-2-sulfonamide (Intermediate 330, 0.58 mmol, 0.07 g), triethylamine (0.86 mmol, 0.12 mL, 86 mg), 2-chloro-1-methylpyridinium iodide (0.45 mmol, 0.116 g) and 4-(Dimethylamino)pyridine (0.06 mmol, 7 mg) and stirred at RT for 3-4 h. The reaction mixture was diluted with dichloromethane and further washed with 25% citric acid solution, water and brine. The organic layer was dried over Na₂SO₄ and concentrated. The crude mass was purified by 60-120 mesh silica (product eluted with 1% MeOH in CHCl₃) to afford 100 mg of the title compound.

| | | Mass spectrum and ¹H | |
|---|---|---|---|
| Compound | Structure | NMR | SM |
| Example 818 | 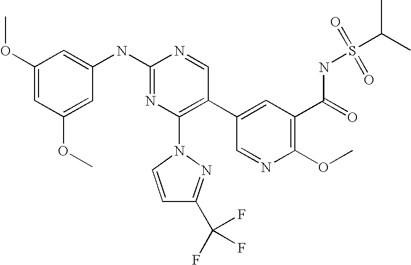 5-{2-[(3,5-dimethoxyphenyl)-amino]-4-[3-(trifluoromethyl)-1H-pyrazol-1-yl]pyrimidin-5-yl}-2-methoxy-N-(propan-2-ylsulfonyl) pyridine-3-carboxamide | MS(ES): 622 (M + 1) for C₂₆H₂₆F₃N₇O₆S. 400 MHz, CDCl₃: δ 1.49 (d, J = 6.92 Hz, 6H), 3.84 (s, 6H), 3.98-3.99 (m, 1H), 4.22 (s, 3H), 6.28 (t, J = 2.08 Hz, 1H), 6.67 (d, J = 2.60 Hz, 1H), 6.88 (d, J = 2.12 Hz, 2H), 7.35 (s, 1H), 8.26 (d, J = 2.48 Hz, 1H), 8.31 (d, J = 2.52 Hz, 1H), 8.48 (s, 1H), 8.49 (d, J = 1.84 Hz, 1H), 9.91 (s, 1H). | Intermediate 330 propane-2-sulfonamide and Example 675 5-{2-[(3,5- dimethoxyphenyl)amino]- 4-[3-(trifluoro-methyl)-1H- pyrazol-1- yl]pyrimidin- 5-yl}-2-methoxy-pyridine-3- carboxylic acid |

### Example 819

### 5-{2-[(3,5-dimethoxyphenyl)amino]-4-[5-methyl-3-(trifluoromethyl)-1H-pyrazol-1-yl]pyrimidin-5-yl}-2-methoxy-N-(propan-2-ylsulfonyl)pyridine-3-carboxamide

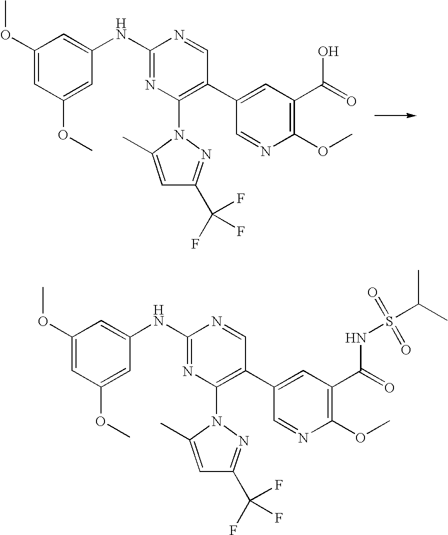

To a solution of 5-{2-[(3,5-dimethoxyphenyl)amino]-4-[5-methyl-3-(trifluoromethyl)-1H-pyrazol-1-yl]pyrimidin-5-yl}-2-methoxypyridine-3-carboxylic acid (Example 677, 0.37 mmol, 0.2 g) in CH₂Cl₂ (10 mL), were added propane-2-sulfonamide (Intermediate 330, 0.56 mmol, 0.07 g), triethylamine (1.1 mmol, 0.15 mL), 2-chloro-1-methylpyridinium iodide (0.45 mmol, 0.116 g) and 4-(Dimethylamino)pyridine (0.06 mmol, 7 mg) and stirred at RT for 3-4 h. The reaction mixture was diluted with dichloromethane and further washed with 25% citric acid solution, water and brine. The organic layer was dried over Na₂SO₄ and concentrated. The crude mass was purified by 60-120 mesh silica (product eluted with 1% MeOH in CHCl₃) to afford 130 mg of the title compound.

| | | Mass spectrum and ¹H | |
|---|---|---|---|
| Compound | Structure | NMR | SM |
| Example 819 | 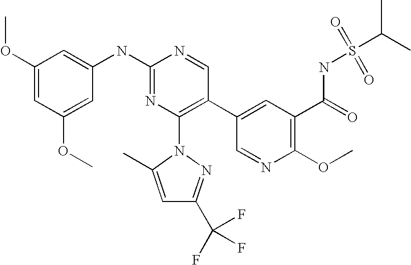 5-{2-[(3,5-dimethoxyphenyl)-amino]-4-[5-methyl-3-(trifluoromethyl)-1H-pyrazol-1-yl]pyrimidin-5-yl}-2-methoxy-N-(propan-2-ylsulfonyl) pyridine-3-carboxamide | MS(ES): 636 (M + 1) for C₂₇H₂₈F₃N₇O₆S. 400 MHz, CDCl₃: δ 1.48 (d, J = 6.92 Hz, 6H), 2.47 (s, 3H), 3.81 (s, 6H), 3.93-3.97 (m, 1H), 4.18 (s, 3H), 6.26 (t, J = 2.08 Hz, 1H), 6.44 (s, 1H), 6.87 (d, J = 2.12 Hz, 2H), 7.36 (s, 1H), 8.03 (d, J = 2.52 Hz, 1H), 8.08 (d, J = 2.52 Hz, 1H), 8.63 (s, 1H), 9.84 (s, 1H). | Intermediate 330 propane-2- sulfonamide and Example 677 5-{2-[(3,5-dimethoxyphenyl) amino]-4-[5-methyl-3- (trifluoromethyl)-1H- pyrazol-1-yl]pyrimidin- 5-yl}-2- methoxypyridine-3- carboxylic acid |

### Example 820

### 5-{2-[(3,5-dimethoxyphenyl)amino]-4-[3-(trifluoromethyl)-1H-pyrazol-1-yl]pyrimidin-5-yl}-2-methoxy-N-{[3-(morpholin-4-yl)propyl]sulfonyl}pyridine-3-carboxamide

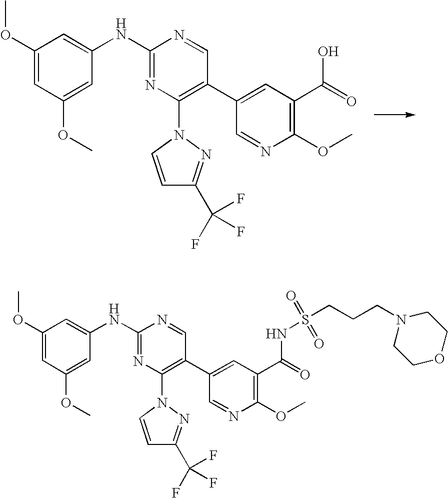

To a solution of 5-{2-[(3,5-dimethoxylphenyl)amino]-4-[3-(trifluoromethyl)-1H-pyrazol-1-yl]pyrimidin-5-yl}-2-methoxypyridine-3-carboxylic acid (Example 675, 0.38 mmol, 0.2 g) and 4-(Dimethylamino)pyridine (0.06 mmol, 7 mg) in CH₂Cl₂ (10 mL), were added 3-(morpholin-4-yl)propane-1-sulfonamide (Intermediate 332, 0.58 mmol, 0.12 g), triethylamine (0.86 mmol, 0.12 mL, 86 mg), 2-chloro-1-methylpyridinium iodide (0.45 mmol, 0.116 g) and stirred at RT for 3-4 h. The reaction mixture was diluted with dichloromethane and further washed with 25% citric acid solution, water and brine. The organic layer was dried over Na₂SO₄ and concentrated. The crude mass was purified by 60-120 mesh silica (product eluted with 1% MeOH in CHCl₃) to afford 70 mg of the title compound.

| | | Mass spectrum and ¹H | |
|---|---|---|---|
| Compound | Structure | NMR | SM |
| Example 820 | 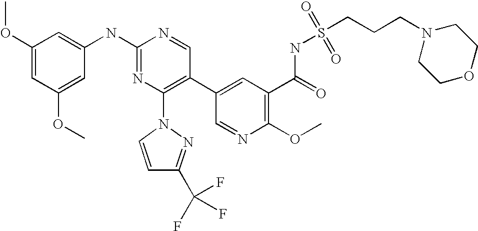 5-{2-[(3,5-dimethoxyphenyl)-amino]-4-[3-(trifluoromethyl)-1H-pyrazol-1-yl] pyrimidin-5-yl}-2-methoxy-N-{[3-(morpholin-4-yl)propyl]sulfonyl}-pyridine-3-carboxamide | MS(ES): 707 (M + 1) for C₃₀H₃₃F₃N₈O₇S. 400 MHz, DMSO-d₆: δ 1.91 (br s, 2H), 2.57-2.63 (m, 5H), 3.39 (m, 3H), 3.61 (s, 4H), 3.74 (s, 6H), 3.91 (s, 3H), 6.20 (s, 1H), 7.02 (s, 1H), 7.10 (s, 2H), 7.76 (s, 1H), 8.08 (s, 1H), 8.41 (s, 1H), 8.74 (s, 1H), 10.18 (s, 1H). | Intermediate 332 3- (morpholin-4-yl) propan-1-sulfonamide and Example 675 5-{2-[(3,5-dimethoxyphenyl) amino]-4-[3-(trifluoro- methyl)-1H-pyrazol- 1-yl]pyrimidin- 5-yl}-2-methoxy- pyridine-3-carboxylic acid |

### Example 821

### 5-{2-[(3,5-dimethoxyphenyl)amino]-4-[5-methyl-3-(trifluoromethyl)-1H-pyrazol-1-yl]pyrimidin-5-yl}-2-methoxy-N-{[3-(morpholin-4-yl)propyl]sulfonyl}pyridine-3-carboxamide

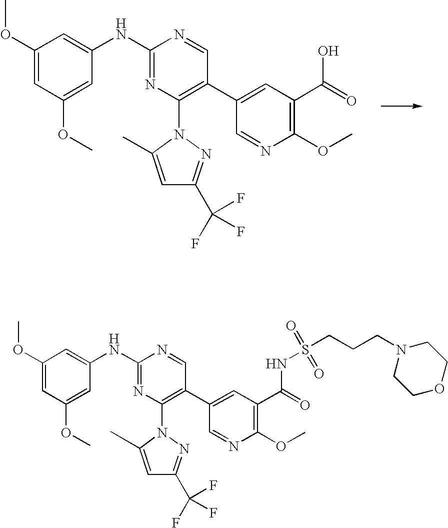

To a solution of 5-{2-[(3,5-dimethoxyphenyl)amino]-4-[5-methyl-3-(trifluoromethyl)-1H-pyrazol-1-yl]pyrimidin-5-yl}-2-methoxypyridine-3-carboxylic acid (Example 677, 0.37 mmol, 0.2 g) and 4-(Dimethylamino)pyridine (0.06 mmol, 7 mg) in CH₂Cl₂ (10 mL) were added 3-(morpholin-4-yl)propane-1-sulfonamide (Intermediate 332, 0.58 mmol, 0.12 g), triethylamine (1.1 mmol, 0.15 mL), 2-chloro-1-methylpyridinium iodide (0.45 mmol, 0.116 g) and stirred at RT for 3-4 h. The reaction mixture was diluted with dichloromethane and further washed with 25% citric acid solution, water and brine. The organic layer was dried over Na₂SO₄ and concentrated. The crude mass was purified by 60-120 mesh silica (product eluted with 3% MeOH in CHCl₃) to afford 130 mg of the title compound.

| | | Mass spectrum and ¹H | |
|---|---|---|---|
| Compound | Structure | NMR | SM |
| Example 821 | 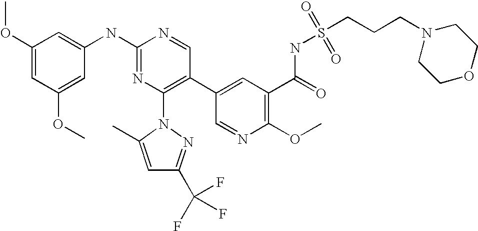 5-{2-[(3,5-dimethoxyphenyl)-amino]-4-[5-methyl-3-(trifluoromethyl)-1H-pyrazol-1- yl]pyrimidin-5-yl}-2-methoxy-N-{[3-(morpholin-4-yl)propyl]sulfonyl} pyridine-3-carboxamide | MS(ES): 721 (M + 1) for C₃₁H₃₅F₃N₈O₇S. 400 MHz, CDCl₃: δ 2.15 (br s, 2H), 2.46 (s, 3H), 22.56 (br s, 6H), 3.66 (t, J = 7.76 Hz, 2H), 3.77 (s, 4H), 3.81 (s, 6H), 4.17 (s, 3H), 6.26 (t, J = 2.04 Hz, 1H), 6.44 (s, 1H), 6.87 (d, J = 2.08 Hz, 2H), 7.35 (s, 1H), 8.05 (d, J = 2.52 Hz, 1H), 8.08 (d, J = 2.52 Hz, 1H), 8.63 (s, 1H). | Intermediate 332 3-(morpholin-4-yl) propane-1- sulfonamide and Example 677 5-{2-[(3,5-dimethoxy- phenyl)amino]- 4-[5-methyl-3- (trifluoromethyl)-1H- pyrazol-1- yl]pyrimidin- 5-yl}-2- methoxypyridine-3- carboxylic acid |

### Example 822

### 5-{2-[(3,5-dimethylphenyl)amino]-4-[3-(trifluoromethyl)-1H-pyrazol-1-yl]pyrimidin-5-yl}-N-(ethylsulfonyl)-2-methoxypyridine-3-carboxamide

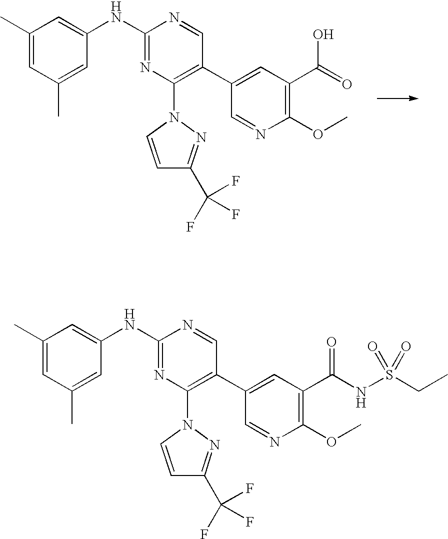

To a solution of 5-{2-[(3,5-dimethylphenyl)amino]-4-[3-(trifluoromethyl)-1H-pyrazol-1-yl]pyrimidin-5-yl}-2-methoxypyridine-3-carboxylic acid (Example 648, 0.52 mmol, 0.250 g) and 4-(Dimethylamino)pyridine (0.104 mmol, 0.013 g) in CH₂Cl₂ (25 mL) were added ethanesulfonamide (Intermediate 328, 1.3 mmol, 0.14 g), triethylamine (1.61 mmol, 0.225 mL), 2-chloro-1-methylpyridinium iodide (0.63 mmol, 0.16 g) and stirred overnight at RT. The reaction mixture was diluted with dichloromethane (50 mL) and further washed with 25% citric acid solution (2×50 mL), water (100 mL) and brine (75 mL). The organic layer was dried over Na₂SO₄ and concentrated. The crude mass was purified by 60-120 mesh silica (product eluted with 1-1.5% methanol/chloroform) to afford 208 mg of white solid with 94% purity by LCMS. This was further purified using RP-HPLC (Atlantis C18 column (19×250 mm, 10 μm); using a binary solvent mixture of 20 mM NH₄OAc (A)/MeOH (B) (0-20 min: 10-65% B, 20-30 min: 65% B and 30-45 min: 65-100% B; 45-50 min: 100% B flow rate of 15 mL/min; Separation was monitored at 210, 254 and 300 nm) to give 110 mg of the title compound.

| | | Mass spectrum and ¹H | |
|---|---|---|---|
| Compound | Structure | NMR | SM |
| Example 822 | 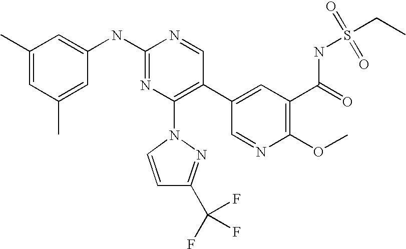 5-{2-[(3,5-dimethylphenyl)amino]-4-[3-(trifluoromethyl)-1H- pyrazol-1-yl]pyrimidin-5-yl}-N-(ethylsulfonyl)-2-methoxy- pyridine-3-carboxamide | MS(ES): 576 (M + 1) for C₂₅H₂₄F₃N₆O₄S. 400 MHz, DMSO-d₆: δ 1.26 (t, J = 7.24 Hz, 3H), 2.28 (s, 6H), 3.46 (q, J = 7.20 Hz, 2H), 3.96 (s, 3H), 6.70 (s, 1H), 7.04 (s, 1H), 7.43 (s, 2H), 7.80 (s, 1H), 8.20 (d, J = 1.72 Hz, 1H), 8.46 (s, 1H), 8.75 (s, 1H), 10.10 (s, 1H), 11.69 (s, 1H). | Intermediate 328 Ethanesulfonamide and Example 648 5-{2-[(3,5- dimethylphenyl)amino]- 4-[3-(trifluoromethyl)-1H- pyrazol-1-yl]pyrimidin- 5-yl}-2- methoxypyridine-3- carboxylic acid |

### Example 823

### 5-{2-[(3,5-dimethylphenyl)amino]-4-[5-methyl-3-(trifluoromethyl)-1H-pyrazol-1-yl[pyrimidin-5-yl}-N-(ethylsulfonyl)-2-methoxypyridine-3-carboxamide

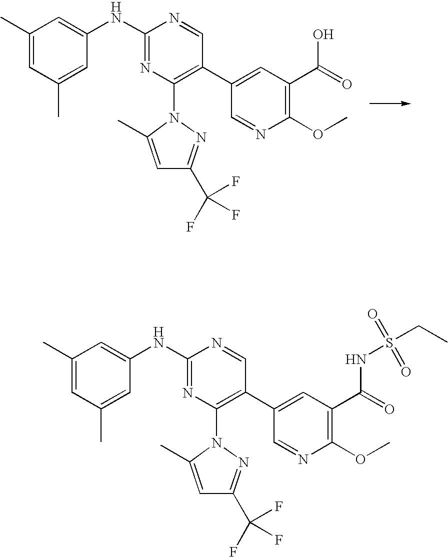

To a solution of 5-{2-[(3,5-dimethylphenyl)amino]-4-[5-methyl-3-(trifluoromethyl)-1H-pyrazol-1-yl]pyrimidin-5-yl}-2-methoxypyridine-3-carboxylic acid (Example 646, 0.3 mmol, 0.150 g) and 4-(Dimethylamino)pyridine (0.06 mmol, 7 mg) in CH₂Cl₂ were added ethanesulfonamide (Intermediate 328, 0.75 mmol, 0.082 g), triethylamine (0.9 mmol, 0.125 mL), 2-chloro-1-methylpyridinium iodide (0.36 mmol, 0.095 g) and stirred at RT for 3-4 h. The reaction mixture was diluted with dichloromethane and further washed with 25% citric acid solution, water and brine. The organic layer was dried over Na₂SO₄ and concentrated. The crude mass was purified by 60-120 mesh silica (product eluted with 1% MeOH/CHCl₃) to afford 130 mg of the title compound.

| | | Mass spectrum and ¹H | |
|---|---|---|---|
| Compound | Structure | NMR | SM |
| Example 823 | 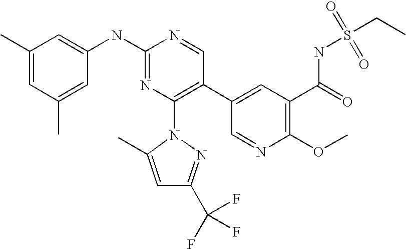 5-{2-[(3,5-dimethylphenyl)amino]-4-[5-methyl-3-(trifluoromethyl)- 1H-pyrazol-1-yl]pyrimidin-5-yl}-N-(ethylsulfoinyl)-2-methoxy- pyridine-3-carboxamide | MS(ES): 590 (M + 1) for C₂₆H₂₆F₃N₇O₄S. 400 MHz, CDCl₃: δ 1.45 (t, J = 7.40 Hz, 3H), 2.35 (s, 6H), 2.50 (s, 3H), 3.57 (q, J = 7.36 Hz, 2H), 4.18 (s, 3H), 6.44 (s, 1H), 6.81 (s, 1H), 7.24 (s, 2H), 7.32 (s, 1H), 8.04 (d, J = 2.56 Hz, 1H), 8.09 (d, J = 2.56 Hz, 1H), 8.61 (s, 1H), 9.94 (s, 1H). | Intermediate 328 Ethanesulfonamide and Example 646 5-{2-[(3,5-dimethylphenyl)amino]- 4-[5-methyl-3-(trifluoromethyl)- 1H-pyrazol-1-yl]pyrimidin- 5-yl}-2- methoxypyridine-3- carboxylic acid |

### Example 824

### 5-{2-[(3,5-dimethylphenyl)amino]-4-[3-(trifluoromethyl)-1H-pyrazol-1-yl]pyrimidin-5-yl}-2-methoxy-N-(propylsulfonyl)pyridine-3-carboxamide

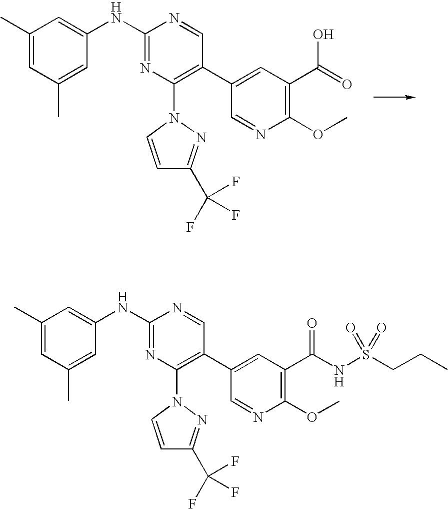

To a solution of 5-{2-[(3,5-dimethylphenyl)amino]-4-[3-(trifluoromethyl)-1H-pyrazol-1-yl]pyrimidin-5-yl}-2-methoxypyridine-3-carboxylic acid (Example 648, 0.52 mmol, 0.250 g) and 4-(Dimethylamino)pyridine (0.61 mmol, 0.075 g) in CH₂Cl₂ (25 mL) were added propane-1-sulfonamide (Intermediate 328, 1.3 mmol, 0.16 g), triethylamine (1.61 mmol, 0.225 mL), 2-chloro-1-methylpyridinium iodide (0.63 mmol, 0.16 g) and stirred overnight at RT. The reaction mixture was diluted with dichloromethane (50 mL) and further washed with 25% citric acid solution (2×50 mL), water (100 mL) and brine (75 mL). The organic layer was dried over Na₂SO₄ and concentrated. The crude mass was purified by 230-400 mesh silica (product eluted with 40-45% ethyl acetate/hexanes) to afford 200 mg of white solid with 81% purity by LCMS. This was further purified using RP-HPLC (Atlantis C18 column (19×250 mm, 10 μm); using a binary solvent mixture of 20 mM NH₄OAc (A)/MeOH (B) (0-20 min: 10-65% B, 20-30 min: 65% B and 30-45 min: 65-100% B; 45-50 min: 100% B flow rate of 15 mL/min; Separation was monitored at 210, 254 and 300 nm) to give 125 mg of Example 824.

| Com- | | Mass spectrum and ¹H | |
|---|---|---|---|
| pound | Structure | NMR | SM |
| Example 824 | 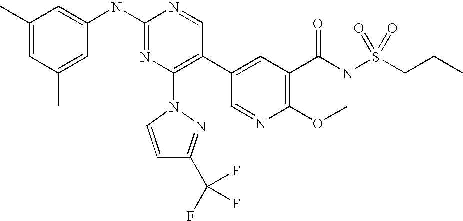 5-{2-[(3,5-dimethylphenyl)amino]-4-[3-(trifluoromethyl)-1H-pyrazol-1-yl] pyrimidin-5-yl}-2-methoxy-N-(propylsulfonyl)pyridine-3-carboxamide | MS(ES): 590 (M + 1) for C₂₆H₂₆F₃N₇O₄S. 400 MHz, DMSO-d₆: δ 1.00 (t, J = 7.36 Hz, 3H), 1.71-1.77 (m, 2H), 2.28 (s, 6H), 3.44 (t, J = 7.64 Hz, 2H), 3.96 (s, 3H), 6.70 (s, 1H), 7.04 (s, 1H), 7.43 (s, 2H), 7.80 (s, 1H), 8.21 (s, 1H), 8.46 (s, 1H), 8.75 (s, 1H), 10.09 (s, 1H), 11.70 (s, 1H). | Intermediate 328 propane-1-sulfonamide and Example 648 5-{2-[(3,5- dimethylphenyl)amino]-4-[3- (trifluoromethyl)-1H-pyrazol-1- yl]pyrimidin-5-yl}-2- methoxypyridine- 3-carboxylic acid |

### Example 825

### 5-{2-[(3,5-dimethylphenyl)amino]-4-[5-methyl-3-(trifluoromethyl)-1H-pyrazol-1-yl]pyrimidin-5-yl}-2-methoxy-N-(propylsulfonyl)pyridine-3-carboxamide

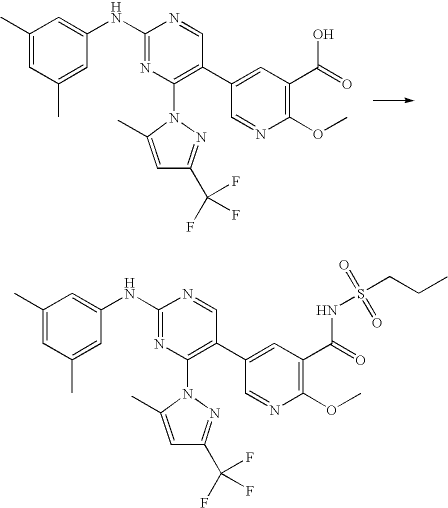

To a solution of 5-{2-[(3,5-dimethylphenyl)amino]-4-[5-methyl-3-(trifluoromethyl)-1H-1-yl]pyrimidin-5-yl}-2-methoxypyridine-3-carboxylic acid (Example 646, 0.3 mmol, 0.150 g) and 4-(Dimethylamino)pyridine (0.06 mmol, 7 mg) in CH₂Cl₂ were added propane-1-sulfonamide (Intermediate 329, 0.75 mmol, 0.092 g), triethylamine (0.9 mmol, 0.125 mL, 92 mg), 2-chloro-1-methylpyridinium iodide (0.37 mmol, 0.095 g) and stirred at RT for 3-4 h. The reaction mixture was diluted with dichloromethane and further washed with 25% citric acid solution, water and brine. The organic layer was dried over Na₂SO₄ and concentrated. The crude mass was purified by 60-120 mesh silica (product eluted with 1% MeOH/CHCl₃) to afford 90 mg of the title compound.

| | | Mass spectrum and ¹H | |
|---|---|---|---|
| Compound | Structure | NMR | SM |
| Example 825 | 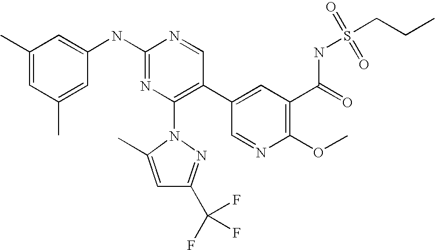 5-{2-[(3,5-dimethylphenyl)-amino]-4-[5-methyl-3-(trifluoro-nl methyl)-1H-pyrazol-1-yl]pyrimidin-5-yl}-2-methoxy-N-(propylsulfonyl)- pyridine-3-carboxamide | MS(ES): 604 (M + 1) for C₂₇H₂₈F₃N₇O₄S. 400 MHz, CDCl₃: δ 1.10 (t, J = 7.48 Hz, 3H), 1.93 (q, J = 7.56 Hz, 2H), 2.35 (s, 6H), 2.50 (s, 3H), 3.50- 3.54 (m, 2H), 4.18 (s, 3H), 6.44 (s, 1H), 6.81 (s, 1H), 7.23 (s, 2H), 7.44 (br s, 1H), 8.04 (d, J = 2.52 Hz, 1H), 8.09 (d, J = 2.52 Hz, 1H), 8.60 (s, 1H), 9.96 (s, 1H). | Intermediate 329 propane-1-sulfonamide and Example 646 5-{2-[(3,5- dimethylphenyl) amino]-4-[5-methyl-3- (trifluoromethyl)- 1H-pyrazol-1-yl] pyrimidin-5-yl}-2- methoxypyridine- 3-carboxylic acid |

### Example 826

### 5-{2-[(3,5-dimethylphenyl)amino]-4-[3-(trifluoromethyl)-1H-pyrazol-1-yl]pyrimidin-5-yl}-2-methoxy-N-(propan-2-ylsulfonyl)pyridine-3-carboxamide

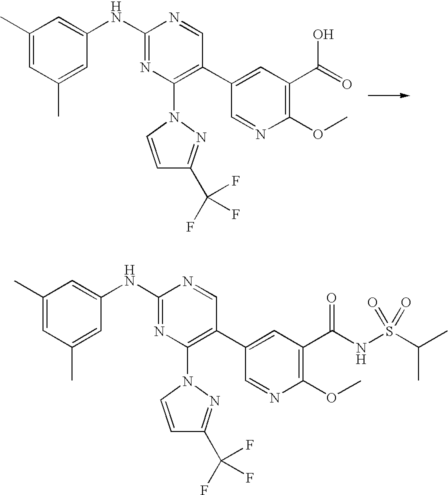

To a solution of 5-{2-[(3,5-dimethylphenyl)amino]-4-[3-(trifluoromethyl)-1H-pyrazol-1-yl]pyrimidin-5-yl}-2-methoxypyridine-3-carboxylic acid (Example 648, 0.412 mmol, 0.200 g) and 4-(Dimethylamino)pyridine (0.082 mmol, 0.010 g) in CH₂Cl₂ (25 mL) were added propane-2-sulfonamide (Intermediate 330, 0.99 mmol, 0.122 g), triethylamine (1.236 mmol, 0.173 mL), 2-chloro-1-methylpyridinium iodide (0.49 mmol, 0.126 g) and stirred overnight at RT. The reaction mixture was diluted with dichloromethane (50 mL) and further washed with 25% citric acid solution (2×50 mL), water (100 mL) and brine (75 mL). The organic layer was dried over Na₂SO₄ and concentrated. The crude mass was purified by 60-120 mesh silica (product eluted with 1.5-2.5% MeOH in CHCl₃) to afford 110 mg of white solid with 86% purity by LCMS. This was further purified using RP-HPLC (kromasil C18 column (50×250 mm, 10 μm); using a binary solvent mixture of 0.1% TFA in water (A)/MeOH (B) (0-20 min: 10-70% B, 20-30 min: 70-80% B and 30-40 min: 80-90% B; 40-50 min: 90% B, 50-55 min: 90-100% B flow rate of 40 mL/min; Separation was monitored at 210 and 290 nm) to give 40 mg of Example 826.

| | | | |
|---|---|---|---|
| | | Mass spectrum and ¹H | |
| Compound | Structure | NMR | SM |

| | | | |
|---|---|---|---|
| Example 826 | 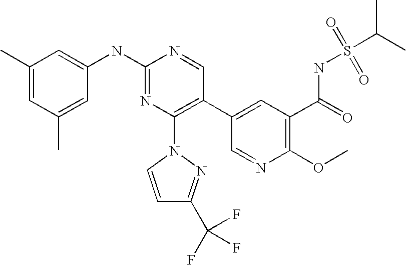 5-{2-[(3,5-dimethylphenyl)amino]-4-[3-(trifluoromethyl)-1H-pyrazol-12-yl] pyrimidin-5-yl}-2-methoxy-N-(propan-2-ylsulfonyl)pyridine-3-carboxamide | MS(ES): 590 (M + 1) for C₂₆H₂₆F₃N₇O₄S. 400 MHz, DMSO-d₆: δ 1.32 (d, J = 6.84 Hz, 6H), 2.27 (s, 6H), 3.70-3.71 (m, 1H), 3.95 (s, 3H), 6.69 (s, 1H), 7.02 (d, J = 2.60 Hz, 1H), 7.42 (s, 2H), 7.75 (d, J = 2.36 Hz, 1H), 8.19 (d, J = 2.40 Hz, 1H), 8.45 (d, J = 1.56 Hz, 1H), 8.74 (s, 1H), 10.08 (s, 1H), 11.66 (s, 1H). | Intermediate 330 propane-2- sulfonamide and Example 648 5-{2-[(3,5-dimethylphenyl)- amino]-4-[3-(trifluoro- methyl)-1H-pyrazol-1- yl]pyrimidin-5-yl}-2- methoxypyridine- 3- carboxylic acid |

### Example 827

### 5-{2-[(3,5-dimethylphenyl)amino]-4-[5-methyl-3-(trifluoromethyl)-1H-pyrazol-1-yl]pyrimidin-5-yl}-2-methoxy-N-(propan-2-ylsulfonyl)pyridine-3-carboxamide

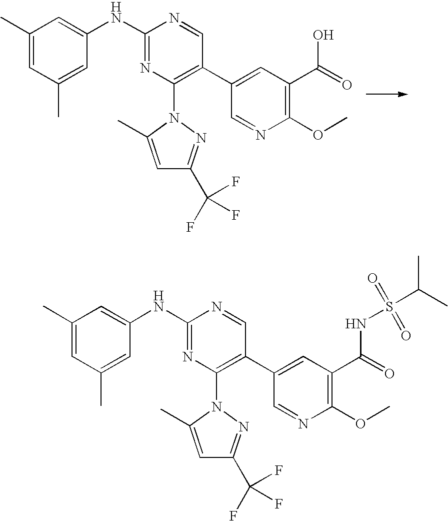

To a solution of 5-{2-[(3,5-dimethylphenyl)amino]-4-[5-methyl-3-(trifluoromethyl)-1H-1-yl]pyrimidin-5-yl}-2-methoxypyridine-3-carboxylic acid (Example 646, 0.3 mmol, 0.150 g) in CH₂Cl₂ (10 mL), were added propane-2-sulfonamide (Intermediate 330, 0.45 mmol, 0.055 g), triethylamine (0.9 mmol, 0.125 mL, 92 mg), 2-chloro-1-methylpyridinium iodide (0.37 mmol, 0.095 g) and 4-(Dimethylamino)pyridine (0.06 mmol, 7 mg), and refluxed at 45° C. for 30′. The reaction mixture was cooled, diluted with dichloromethane and further washed with 25% citric acid solution, water and brine. The organic layer was dried over Na₂SO₄ and concentrated. The crude mass was purified by 60-120 mesh silica (product eluted with 1% MeOH/CHCl₃) to afford 95 mg of the title compound.

| | | Mass spectrum and ¹H | |
|---|---|---|---|
| Compound | Structure | NMR | SM |
| Example 827 | 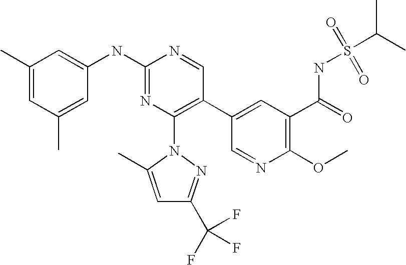 5-{2-[(3,5-dimethylphenyl)amino]-4-[5-methyl-3- (trifluoromethyl)-1H-pyrazol-1-yl]pyrimidin-5-yl}-2- methoxy-N-(propan-2-ylsulfonyl)pyridine-3-carboxamide | MS(ES): 604 (M + 1) for C₂₇H₂₈F₃N₇O₄S. 400 MHz, CDCl₃: δ 1.48 (d, J = 6.84 Hz, 6H), 2.35 (s, 6H), 2.50 (s, 3H), 3.95 (t, J = 6.80 Hz, 1H), 4.18 (s, 3H), 6.44 (s, 1H), 6.81 (s, 1H), 7.24 (s, 2H), 7.34 (br s, 1H), 8.02 (s, 1H), 8.09 (d, J = 2.16 Hz, 1H), 8.64 (br s, 1H), 9.85 (s, 1H). | Intermediate 330 propane-2-sulfonamide and Example 646 5-{2-[(3,5-dimethylphenyl)- amino]-4-[5- methyl-3- (trifluoro- methyl)-1H-pyrazol-1- yl]pyrimidin- 5-yl}-2- methoxypyridine-3- carboxylic acid |

### Example 828

### 5-{2-[(3,5-dimethylphenyl)amino]-4-[3-(trifluoromethyl)-1H-pyrazol-1-yl]pyrimidin-5-yl}-2-methoxy-N-{[3-(morpholin-4-yl)propyl]sulfonyl}pyridine-3-carboxamide

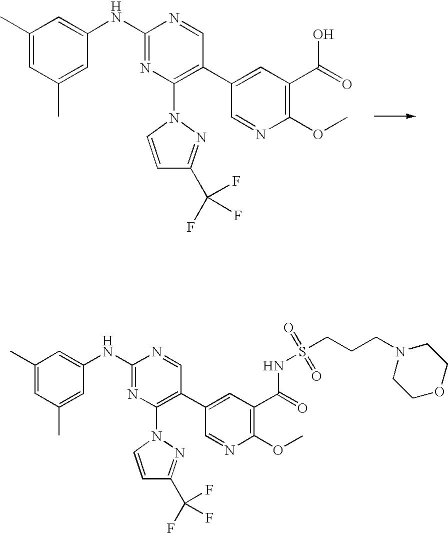

To a solution of 5-{2-[(3,5-dimethylphenyl)amino]-4-[3-(trifluoromethyl)-1H-pyrazol-1-yl]pyrimidin-5-yl}-2-methoxypyridine-3-carboxylic acid (Example 648, 0.52 mmol, 0.250 g) and 4-(Dimethylamino)pyridine (0.104 mmol, 0.013 g) in CH₂Cl₂ (25 mL) were added 3-(morpholin-4-yl)propane-1-sulfonamide (Intermediate 332, 1.25 mmol, 0.26 g), triethylamine (1.61 mmol, 0.225 mL), 2-chloro-1-methylpyridinium iodide (0.63 mmol, 0.16 g) and stirred overnight at RT. The reaction mixture was diluted with dichloromethane (50 mL) and further washed with 25% citric acid solution (2×50 mL), water (100 mL) and brine (75 mL). The organic layer was dried over Na₂SO₄ and concentrated. The crude mass was purified by 60-120 mesh silica (product eluted with 5-7% methanol/chloroform) to afford 170 mg of Example 828.

| | | Mass spectrum and ¹H | |
|---|---|---|---|
| Compound | Structure | NMR | SM |
| Example 828 | 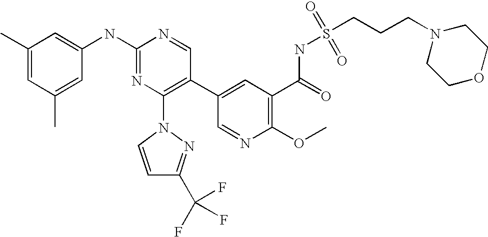 5-{2-[(3,5-dimethylphenyl)amino]-4-[3-(trifluoromethyl)-1H-pyrazol-1-yl] pyrimidin-5-yl}-2-methoxy-N-{[3-(morpholin-4-yl)propyl]sulfonyl}-pyridine-3- carboxamide | MS(ES): 675 (M + 1) for C₃₀H₃₃F₃N₈O₅S. 400 MHz, DMSO-d₆: δ 1.92 (t, J = 7.40 Hz, 2H), 2.26 (s, 6H), 2.63 (s, 6H), 3.42 (t, J = 7.56 Hz, 2H), 3.61 (s, 4H), 3.91 (s, 3H), 6.68 (s, 1H), 7.02 (d, J = 2.48 Hz, 1H), 7.41 (s, 2H), 7.75 (d, J = 2.32 Hz, 1H), 8.09 (d, J = 2.40 Hz, 1H), 8.43 (s, 1H), 8.71 (s, 1H), 10.08 (s, 1H). | Intermediate 332 3- (morpholin-4-yl) propan-2-sulfonamide and Example 648 5-{2-[(3,5-dimethylphenyl) amino]-4-[3-(trifluoromethyl)- 1H-pyrazol-1-yl] pyrimidin-5-yl}-2- methoxypyridine- 3-carboxylic acid |

### Example 829

### 5-{2-[(3,5-dimethylphenyl)amino]-4-[5-methyl-3-(trifluoromethyl)-1H-pyrazol-1-yl]pyrimidin-5-yl}-2-methoxy-N-{[3-(morpholin-4-yl)propyl]sulfonyl}pyridine-3-carboxamide

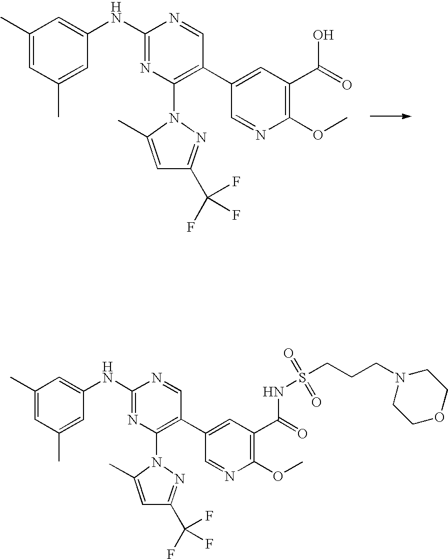

To a solution of 5-{2-[(3,5-dimethylphenyl)amino]-4-[5-methyl-3-(trifluoromethyl)-1H-pyrazol-1-yl]pyrimidin-5-yl}-2-methoxypyridine-3-carboxylic acid (Example 646, 0.4 mmol, 0.2 g) and 4-(Dimethylamino)pyridine (0.08 mmol, 9 mg) in CH₂Cl₂ were added 3-(morpholin-4-yl)propane-1-sulfonamide (Intermediate 332, 0.8 mmol, 0.17 g), triethylamine (1.2 mmol, 0.16 mL, 121 mg), 2-chloro-1-methylpyridinium iodide (0.48 mmol, 0.122 g) and stirred at RT for 3-4 h. The reaction mixture was diluted with dichloromethane and further washed with 25% citric acid solution, water and brine. The organic layer was dried over Na₂SO₄ and concentrated. The crude mass was purified by 60-120 mesh silica (product eluted with 4% MeOH/CHCl₃) to afford 120 mg of the title compound.

| | | Mass spectrum and ¹H | |
|---|---|---|---|
| Compound | Structure | NMR | SM |
| Example 829 | 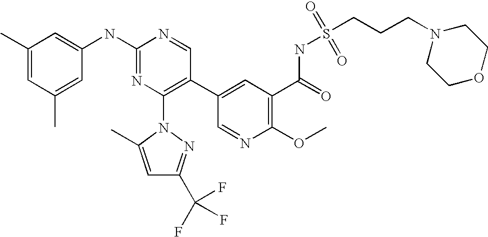 5-{2-[(3,5-dimethylphenyl)amino]-4-[5-methyl-3-(trifluoromethyl)-1H-pyrazol-1-yl] pyrimidin-5-yl}-2-methoxy-N-{[3-(morpholin-4-yl)propyl]sulfonyl}-pyridine-3- carboxamide | MS(ES): 689 (M + 1) for C₃₁H₃₅F₃N₈O₅S. 400 MHz, CDCl₃: δ 1.93 (t, J = 7.20 Hz, 2H), 2.26 (s, 6H), 2.35 (s, 3H), 2.61-2.68 (m, 6H), 3.38- 3.42 (m, 2H), 3.63 (br s, 4H), 3.89 (s, 3H), 6.69 (s, 1H), 6.76 (s, 1H), 7.37 (s, 2H), 7.62 (d, J = 2.08 Hz, 1H), 7.90 (d, J = 2.12 Hz, 1H), 8.88 (s, 1H), 10.11 (s, 1H). | Intermediate 332 3-(morpholin-4- yl)propan-1- sulfonamide and Example 646 5-{2-[(3,5- dimethylphenyl) amino]-4-[5- methyl-3- (trifluoromethyl)- 1H-pyrazol-1- yl]pyrimidin-5- yl}-2- methoxypyridine- 3-carboxylic acid |

### Example 830

### 5-{2-[(3,5-dimethoxyphenyl)amino]-4-[5-methyl-3-(trifluoromethyl)-1H-pyrazol-1-yl]pyrimidin-5-yl}-1,2-benzothiazol-3(2H)-one 1,1-dioxide

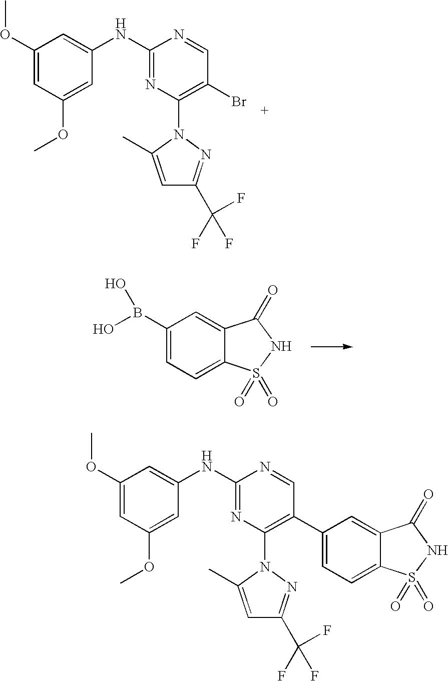

A solution of 5-bromo-N-(3,5-dimethoxyphenyl)-4-[5-methyl-3-(trifluoromethyl)-1H-pyrazol-yl]pyrimidin-2-amine (Intermediate 216, 0.96 mmol, 440 mg), (1,1-dioxido-3-oxo-2,3-dihydro-1,2-benzothiazol-5-yl)boronic acid (Intermediate 335, 1.32 mmol, 300 mg), [1,1′-bis(diphenylphosphino)ferrocene]dichloropalladium(II) complex with CH₂Cl₂ (0.19 mmol, 158 mg) and sodium carbonate (0.96 mmol, 103 mg) in acetonitrile (5 mL)/water (1 mL) was degassed and heated to 90° C. for 20 min under nitrogen. Solvent was removed in vacuo and the residue obtained was taken in ethyl acetate, washed with water and brine, dried over sodium sulfate and concentrated. The crude mass was purified by silica gel column chromatography (60-120 mesh) using 10% methanol/chloroform to yield 157 mg of the title compound.

| | | Mass spectrum and ¹H | |
|---|---|---|---|
| Compound | Structure | NMR | SM |
| Example 830 | 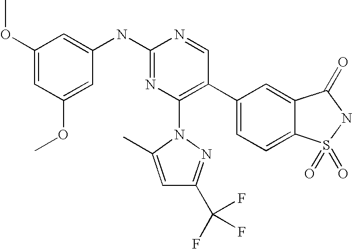 5-{2-[(3,5-dimethoxyphenyl)-amino]-4-[5-methyl-3- (trifluoromethyl)-1H-pyrazol-1-yl]pyrimidin-5-yl 56 -1,2-benzothiazol-3(2H)-one 1,1-dioxide | MS(ES): 561 (M + 1) for C₂₄H₁₉F₃N₆O₅S. 400 MHz, DMSO-d₆: δ 2.20 (s, 3H), 3.71 (s, 6H), 6.20 (t, J = 2.12 Hz, 1H), 6.70 (s, 1H), 7.05 (d, J = 2.08 Hz, 2H), 7.19 (dd, J = 1.44, 7.82 Hz, 1H), 7.28 (s, 1H), 7.56 (d, J = 7.80 Hz, 1H), 8.91 (s, 1H), 10.24 (s, 1H). | Intermediate 216 5-bromo-N-(3,5- dimethoxyphenyl)-4-[5- methyl-3-(trifluoro- methyl)-1H-pyrazol-1- yl]pyrimidin-2-amine |

### Example 831

### 5-{2-[(3,5-dimethoxyphenyl)amino]-4-[3-(trifluoromethyl)-1H-pyrazol-1-yl]pyrimidin-5-yl}-1,2-benzothiazol-3(2H)-one 1,1-dioxide

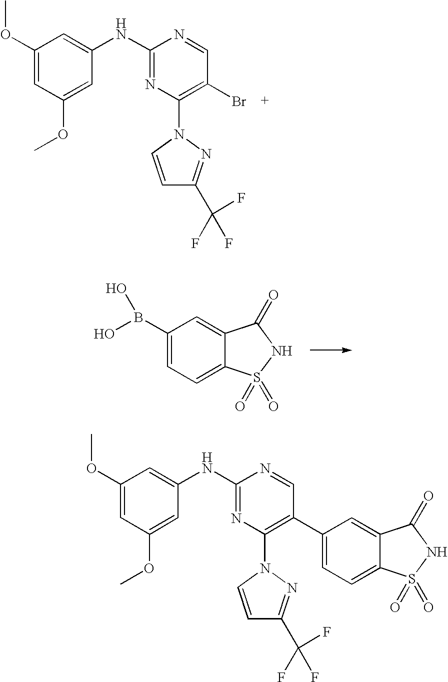

A solution of 5-bromo-N-(3,5-dimethoxyphenyl)-4-[3-(trifluoromethyl)-1H-pyrazol-1-yl]pyrimidin-2-amine (Intermediate 215, 0.97 mmol, 430 mg), (1,1-dioxido-3-oxo-2,3-dihydro-1,2-benzothiazol-5-yl)boronic acid (Intermediate 335, 1.32 mmol, 300 mg), [1,1′-bis(diphenylphosphino)ferrocene]dichloropalladium(II) complex with CH₂Cl₂ (0.19 mmol, 160 mg) and sodium carbonate (0.97 mmol, 103 mg) in acetonitrile (5 mL)/water (1 mL) was degassed and heated to 90° C. for 20 min under nitrogen. Solvent was removed in vacuo and the residue obtained was taken in ethyl acetate, washed with water and brine, dried over sodium sulfate and concentrated. The crude mass was purified by silica gel column chromatography (60-120 mesh) using 9% methanol/chloroform to yield 190 mg of the title compound.

| | | Mass spectrum and ¹H | |
|---|---|---|---|
| Compound | Structure | NMR | SM |
| Example 831 | 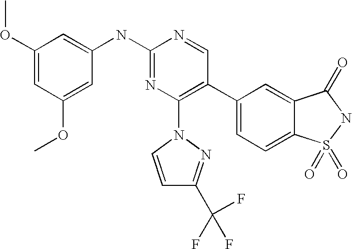 5-{2-[(3,5- dimethoxyphenyl) amino]-4-[3- (trifluoromethyl)-1H- pyrazol-1- yl]pyrimidin-5-yl}-1,2- benzothiazol-3(2H)- one 1,1-dioxide | MS(ES): 547 (M + 1) for C₂₃H₁₇F₃N₆O₅S. 400 MHz, DMSO-d₆: δ 3.73 (s, 6H), 6.19 (t, J = 2.20 Hz, 1H), 6.97 (d, J = 2.64 Hz, 1H), 7.12 (d, J = 2.20 Hz, 2H), 7.32 (dd, J = 1.56, 7.78 Hz, 1H), 7.37 (d, J = 0.92 Hz, 1H), 7.60 (d, J = 7.80 Hz, 1H), 8.29 (t, J = 1.68 Hz, 1H), 8.76 (s, 1H), 10.21 (s, 1H). | Intermediate 215 5-bromo-N- (3,5- dimethoxy- phenyl)-4-[3- (trifluoro- methyl)-1H- pyrazol-1- yl]pyrimidin- 2-amine |

### Example 832

### 5-{2-[(3,5-dimethylphenyl)amino]-4-[5-methyl-3-(trifluoromethyl)-1H-pyrazol-1-yl]pyrimidin-5-yl}-1,2-benzothiazol-3(2H)-one 1,1-dioxide

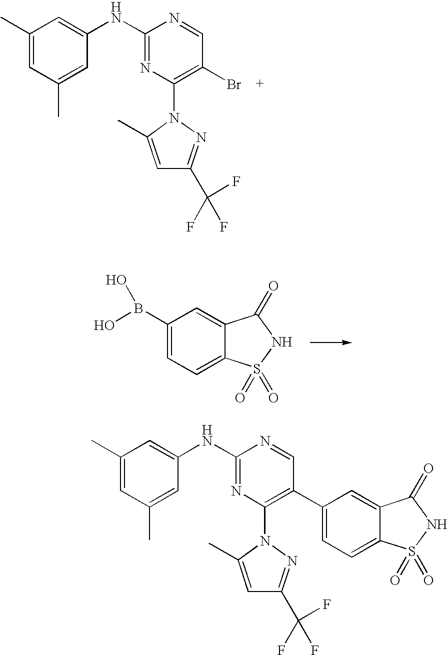

A solution of 5-bromo-N-(3,5-dimethylphenyl)-4-[5-methyl-3-(trifluoromethyl)-1H-pyrazol-1-yl]pyrimidin-2-amine (Intermediate 218, 0.97 mmol, 413 mg), (1,1-dioxido-3-oxo-2,3-dihydro-1,2-benzothiazol-5-yl)boronic acid (Intermediate 335, 1.32 mmol, 300 mg), [1,1′-bis(diphenylphosphino)ferrocene]dichloropalladium(II) complex with CH₂Cl₂ (0.19 mmol, 158 mg) and sodium carbonate (0.97 mmol, 103 mg) in acetonitrile (5 mL)/water (1 mL) was degassed and heated to 90° C. for 20 min under nitrogen. Solvent was removed in vacuo and the residue was taken in ethyl acetate, washed with water and brine, dried over sodium sulfate and concentrated. The crude mass was purified by silica gel column chromatography (60-120 mesh) using 10% methanol/chloroform to yield 195 mg of the title compound.

| | | Mass spectrum and ¹H | |
|---|---|---|---|
| Compound | Structure | NMR | SM |
| Example 832 | 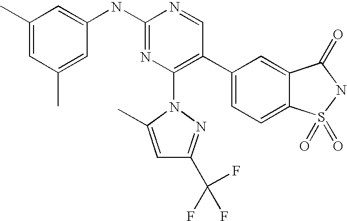 5-{2-[(3,5- dimethylphenyl)amino]- 4-[5-methyl-3- (trifluoromethyl)-1H- pyrazol-1- yl]pyrimidin-5-yl}-1,2- benzothiazol-3(2H)- one 1,1-dioxide | MS(ES): 529 (M + 1) for C₂₄H₁₉F₃N₆O₃S. 400 MHz, DMSO-d₆: δ 2.24 (s, 6H), 2.26 (s, 3H), 6.69 (d, J = 6.16 Hz, 2H), 7.19 (d, J = 7.80 Hz, 1H), 7.26 (s, 1H), 7.37 (s, 2H), 7.55 (d, J = 7.76 Hz, 1H), 8.86 (s, 1H), 10.12 (s, 1H). | Intermediate 218 5-bromo-N- (3,5- dimethyl- phenyl)-4-[5- methyl-3- (trifluoro- methyl)-1H- pyrazol-1- yl]pyrimidin- 2-amine |

### Example 833

### 5-{2-[(3,5-dimethylphenyl)amino]-4-[3-(trifluoromethyl)-1H-pyrazol-1-yl]pyrimidin-5-yl}-1,2-benzothiazol-3(2H)-one 1,1-dioxide

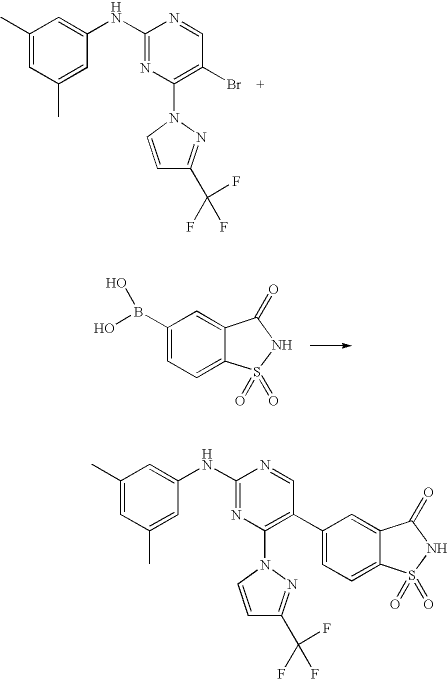

A solution of 5-bromo-N-(3,5-dimethylphenyl)-4-[3-(trifluoromethyl)-1H-pyrazol-1-yl]pyrimidin-2-amine (Intermediate 217, 0.97 mmol, 399 mg), (1,1-dioxido-3-oxo-2,3-dihydro-1,2-benzothiazol-5-yl)boronic acid (Intermediate 335, 1.32 mmol, 300 mg), [1,1′-bis(diphenylphosphino)ferrocene]dichloropalladium(II) complex with CH₂Cl₂ (0.19 mmol, 158 mg) and sodium carbonate (0.97 mmol, 103 mg) in acetonitrile (5 mL)/water (1 mL) was degassed and heated to 90° C. for 20 min under nitrogen. Solvent was removed in vacuo and the residue obtained was taken in ethyl acetate, washed with water and brine, dried over sodium sulfate and concentrated. The crude mass was purified by silica gel column chromatography (60-120 mesh) using 9% methanol/chloroform to yield 200 mg of the title compound.

| | | Mass spectrum and ¹H | |
|---|---|---|---|
| Compound | Structure | NMR | SM |
| Example 833 | 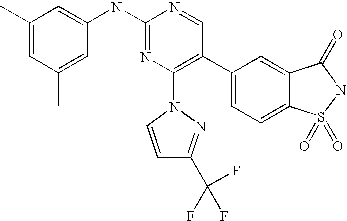 5-{2-[(3,5- dimethylphenyl)amino]- 4-[3-(trifluoromethyl)- 1H-pyrazol-1- yl]pyrimidin-5-yl}-1,2- benzothiazol-3(2H)- one 1,1-dioxide | MS(ES): 515 (M + 1) for C₂₃H₁₇F₃N₆O₃S. 400 MHz, DMSO-d₆: δ 2.26 (s, 6H), 6.67 (s, 1H), 6.97 (d, J = 2.44 Hz, 1H), 7.33 (d, J = 1.20 Hz, 1H), 7.35 (s, 1H), 7.43 (s, 2H), 7.59 (d, J = 7.76 Hz, 1H), 8.32 (s, 1H), 8.73 (s, 1H), 10.11 (s, 1H). | Intermediate 217 5-bromo-N- (3,5- dimethylphenyl)- 4-[3- (trifluoromethyl)- 1H- pyrazol-1- yl]pyrimidin- 2-amine |

### Example 834

### N-{[2-(acetylamino)-4-methyl-1,3-thiazol-5-yl]sulfonyl}-5-{2-[(3,5-dimethoxyphenyl)amino]-4-[5-methyl-3-(trifluoromethyl)-1H-pyrazol-1-yl]pyrimidin-5-yl}-2-methoxypyridine-3-carboxamide

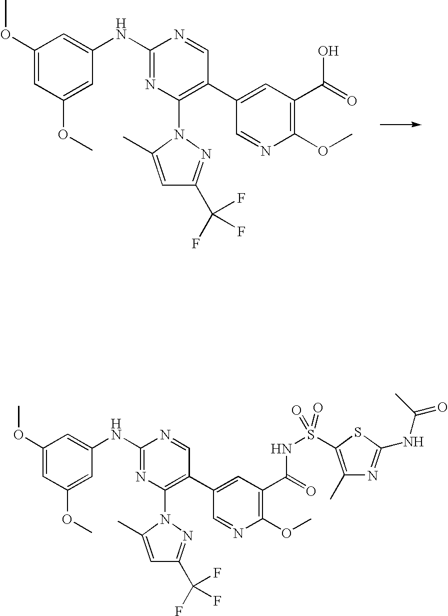

To a solution of 5-{2-[(3,5-dimethoxyphenyl)amino]-4-[5-methyl-3-(trifluoromethyl)-1H-pyrazol-1-yl]pyrimidin-5-yl}-2-methoxypyridine-3-carboxylic acid (Example 675, 0.19 mmol, 0.100 g) and N-(4-methyl-5-sulfamoyl-1,3-thiazol-2-yl)acetamide (Intermediate 336, 0.38 mmol, 0.089 g) in CH₂Cl₂ (25 mL), were added triethylamine (0.57 mmol, 0.08 mL), 2-chloro-1-methylpyridinium iodide (0.23 mmol, 58 mg) and 4-(Dimethylamino)pyridine (0.038 mmol, 5 mg) and stirred for 4-5 h at RT. The reaction mixture was diluted with dichloromethane (50 mL) and further washed with 25% citric acid solution (2×50 mL), water (100 mL) and brine (75 mL). The organic layer was dried over Na₂SO₄ and concentrated. The crude mass was purified by 60-120 mesh silica (product eluted with 4-5% methanol/chloroform) to afford 110 mg of white solid with 89% purity by LCMS. This was further purified using RP-HPLC (Kromasil C18 column (50×250 mm, 10 μm); using a binary solvent mixture of 0.1% TFA in water(A)/MeOH (B) (0-20 min: 10-70% B, 20-30 min: 70-80% B and 30-40 min: 80% B; 40-45 min: 80-100% B, 45-50 min: 100% B, 50-52 min: 100-10 B, flow rate of 40 mL/min; Separation was monitored at 210 and 300 nm) to give 55 mg of the title compound.

| | | Mass spectrum and ¹H | |
|---|---|---|---|
| Compound | Structure | NMR | SM |
| Example 834 | 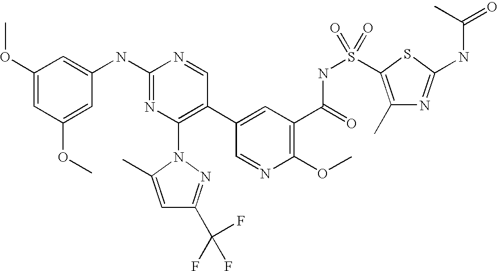 N-{[2-(acetylamino)-4- methyl-1,3-thiazol-5- yl]sulfonyl}-5-{2- [(3,5- dimethoxyphenyl) amino]-4-[5-methyl-3- (trifluoromethyl)-1H- pyrazol-1- yl]pyrimidin-5-yl}-2- methoxypyridine-3- carboxamide | MS(ES): 748 (M + 1) for C₃₀H₂₈F₃N₉O₇S₂. 400 MHz, DMSO-d₆: δ 2.20 (s, 3H), 2.30 (s, 3H), 2.54 (s, 3H), 3.72 (s, 6H), 3.90 (s, 3H), 6.21 (s, 1H), 6.72 (s, 1H), 7.04 (d, J = 1.36 Hz, 2H), 7.59 (d, J = 2.08 Hz, 1H), 7.97 (d, J = 1.72 Hz, 1H), 8.91 (s, 1H), 10.21 (s, 1H), 12.41 (s, 1H), 12.69 (s, 1H). | Intermediate 336 N-(4- methyl-5- sulfamoyl- 1,3-thiazol- 2- yl)acetamide |

### Example 835

### 5-{2-[(3,5-dimethoxyphenyl)amino]-4-[5-methyl-3-(trifluoromethyl)-1H-pyrazol-1-yl]pyrimidin-5-yl}-2-methoxy-N-[(2,2,2-trifluoroethyl)sulfonyl]pyridine-3-carboxamide

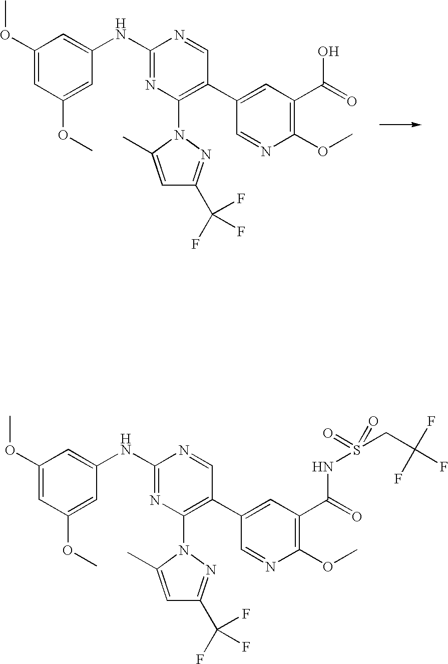

To a solution of 5-{2-[(3,5-dimethoxyphenyl)amino]-4-[5-methyl-3-(trifluoromethyl)-1H-pyrazol-1-yl]pyrimidin-5-yl}-2-methoxypyridine-3-carboxylic acid (Example 675, 0.19 mmol, 0.100 g) and 2,2,2-trifluoroethanesulfonamide (Intermediate 337, 0.43 mmol, 0.07 g) in CH₂Cl₂ (25 mL), were added triethylamine (0.94 mmol, 0.14 mL), 2-chloro-1-methylpyridinium iodide (0.23 mmol, 60 mg) and 4-(Dimethylamino)pyridine (0.08 mmol, 10 mg) and stirred for 1 h at RT. The reaction mixture was diluted with dichloromethane (50 mL) and further washed with 25% citric acid solution (2×50 mL), water (100 mL) and brine (75 mL). The organic layer was dried over Na₂SO₄ and concentrated. The crude mass was purified by 60-120 mesh silica (product eluted with 2% methanol/chloroform) to afford 45 mg of the title compound as a white solid.

| | | Mass spectrum and ¹H | |
|---|---|---|---|
| Compound | Structure | NMR | SM |
| Example 835 | 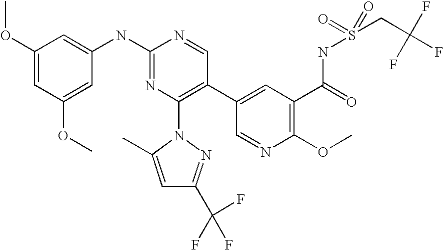 5-{2-[(3,5- dimethoxyphenyl)- amino]-4-[5-methyl-3- (trifluoromethyl)-1H- pyrazol-1- yl]pyrimidin-5-yl}-2- methoxy-N-[(2,2,2- trifluoroethyl)sulfonyl] pyridine-3- carboxamide | MS(ES): 676 (M + 1) for C₂₆H₂₃F₆N₇O₆S. 400 MHz, DMSO-d₆: δ 2.30 (s, 3H), 3.73 (s, 6H), 3.92 (s, 3H), 4.79-4.80 (m, 2H), 6.22 (s, 1H), 6.75 (s, 1H), 7.06 (d, J = 1.68 Hz, 2H), 7.60 (d, J = 2.32 Hz, 1H), 8.05 (s, 1H), 8.93 (s, 1H), 10.23 (s, 1H). | Intermediate 337 2,2,2- trifluoro- ethanesulfonamide |

### Example 836

### 5-{2-[(3,5-dimethoxyphenyl)amino]-4-[5-methyl-3-(trifluoromethyl)-1H-pyrazol-1-yl]pyrimidin-5-yl}-N-[(3,5-dimethyl-1,2-oxazol-4-yl)sulfonyl]-2-methoxypyridine-3-carboxamide

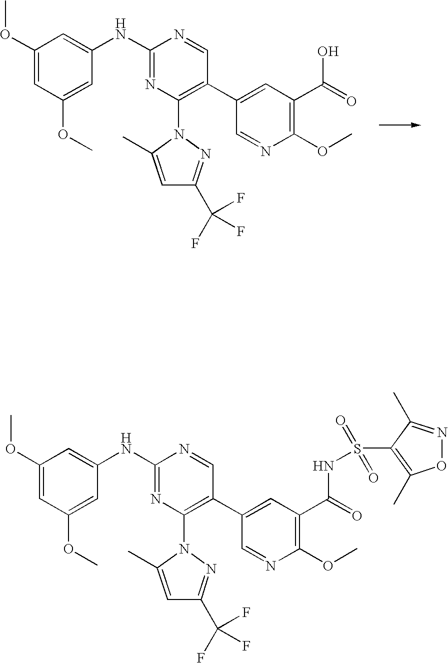

To a solution of 5-{2-[(3,5-dimethoxyphenyl)amino]-4-[5-methyl-3-(trifluoromethyl)-1H-pyrazol-1-yl]pyrimidin-5-yl}-2-methoxypyridine-3-carboxylic acid (Example 675, 0.28 mmol, 0.15 g) and 3,5-dimethyl-1,2-oxazole-4-sulfonamide (Intermediate 338, 0.42 mmol, 74 mg) in CH₂Cl₂ (10 mL), were added triethylamine (0.84 mmol, 0.12 mL), 2-chloro-1-methylpyridinium iodide (0.3 mmol, 78 mg) and 4-(Dimethylamino)pyridine (0.05 mmol, 6 mg) and stirred for 2 h at RT. The reaction mixture was diluted with dichloromethane (50 mL) and further washed with 25% citric acid solution, water and brine. The organic layer was dried over Na₂SO₄ and concentrated. The crude mass was purified by 60-120 mesh silica (product eluted with 2% methanol/chloroform) to afford 85 mg of the title compound.

| | | Mass spectrum and ¹H | |
|---|---|---|---|
| Compound | Structure | NMR | SM |
| Example 836 | 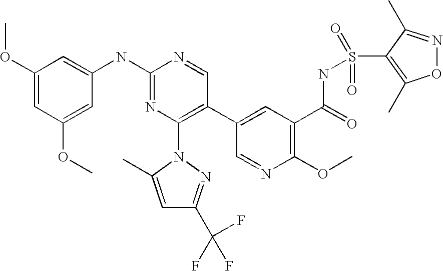 5-{2-[(3,5- dimethoxyphenyl) amino]-4-[5-methyl-3- (trifluoromethyl)-1H- pyrazol-1- yl]pyrimidin-5-yl}-N- [(3,5-dimethyl-1,2- oxazol-4-yl)sulfonyl]- 2-methoxypyridine-3- carboxamide | MS(ES): 689 (M + 1) for C₂₉H₂₇F₃N₈O₇S. 400 MHz, DMSO-d₆: δ 2.29 (s, 3H), 2.40 (s, 3H), 2.68 (s, 3H), 3.72 (s, 6H), 3.90 (s, 3H), 6.21 (d, J = 2.08 Hz, 1H), 6.74 (s, 1H), 7.04 (d, J = 1.96 Hz, 2H), 7.60 (d, J = 2.36 Hz, 1H), 7.97 (s, 1H), 8.91 (s, 1H), 10.22 (s, 1H), 12.62 (br s, 1H). | Intermediate 338 3,5- dimethyl- 1,2-oxazole- 4- sulfonamide |

### Example 837

### 5-{2-[(3,5-dimethoxyphenyl)amino]-4-[5-methyl-3-(trifluoromethyl)-1H-pyrazol-1-yl]pyrimidin-5-yl}-N-[(2,4-dimethyl-1,3-thiazol-5-yl)sulfonyl]-2-methoxypyridine-3-carboxamide

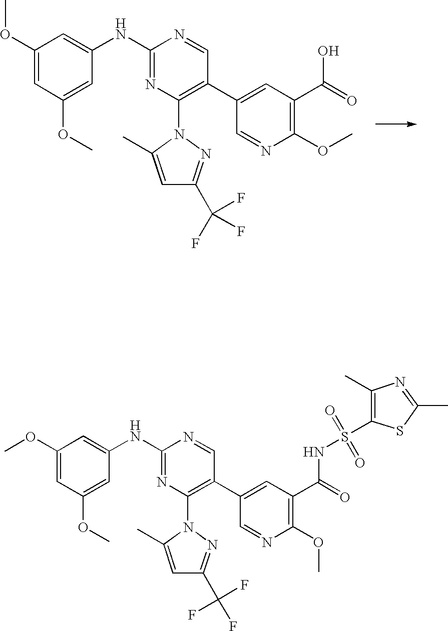

To a solution of 5-{2-[(3,5-dimethoxyphenyl)amino]-4-[5-methyl-3-(trifluoromethyl)-1H-pyrazol-1-yl]pyrimidin-5-yl}-2-methoxypyridine-3-carboxylic acid (Example 675, 0.37 mmol, 0.200 g) in CH₂Cl₂ (10 mL), were added 2,4-dimethyl-1,3-thiazole-5-sulfonamide (Intermediate 339, 0.56 mmol, 0.11 g), triethylamine (0.84 mmol, 0.12 mL), 2-chloro-1-methylpyridinium iodide (0.44 mmol, 0.11 g) and 4-(Dimethylamino)pyridine (0.05 mmol, 6 mg) and stirred at RT for 2 h. The reaction mixture was diluted with dichloromethane and further washed with 10% citric acid solution, water and brine. The organic layer was dried over Na₂SO₄ and concentrated. This was further purified using RP-HPLC (Kromasil C18 column (250×50 mm, 10 μm); using a binary solvent mixture of 10 mM NH₄OAc (A)/MeOH (B) (0-20 min: 20-70% B, 20-30 min: 70-80% B and 30-35 min: 80-100% B; 35-40 min: 100% B flow rate of 40 mL/min; Separation was monitored at 210, 254 and 300 nm) to afford 100 mg of the title compound.

| | | Mass spectrum and ¹H | |
|---|---|---|---|
| Compound | Structure | NMR | SM |
| Example 837 | 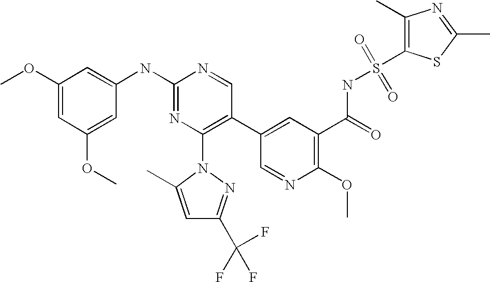 5-{2-[(3,5- dimethoxyphenyl)amino]- 4-[5-methyl-3- (trifluoromethyl)-1H- pyrazol-1- yl]pyrimidin-5-yl}-N- [(2,4-dimethyl-1,3- thiazol-5-yl)sulfonyl]- 2-methoxypyridine-3- carboxamide | MS(ES): 705 (M + 1) for C₂₉H₂₇F₃N₈O₆S₂. 400 MHz, DMSO-d₆: δ 2.30 (s, 3H), 2.58 (s, 3H), 2.68 (s, 3H), 3.72 (s, 6H), 3.90 (s, 3H), 6.21 (t, J = 2.16 Hz, 1H), 6.74 (s, 1H), 7.04 (d, J = 2.08 Hz, 2H), 7.60 (d, J = 2.44 Hz, 1H), 7.96 (s, 1H), 8.91 (s, 1H), 10.21 (s, 1H), 12.54 (s, 1H). | Intermediate 339 2,4- dimethyl- 1,3-thiazole- 5- sulfonamide |

### Example 838

### 5-{2-[(3,5-dimethoxyphenyl)amino]-4-[5-methyl-3-(trifluoromethyl)-1H-pyrazol-1-yl]pyrimidin-5-yl}-2-methoxy-N-{[(methylsulfonyl)methyl]sulfonyl}pyridine-3-carboxamide

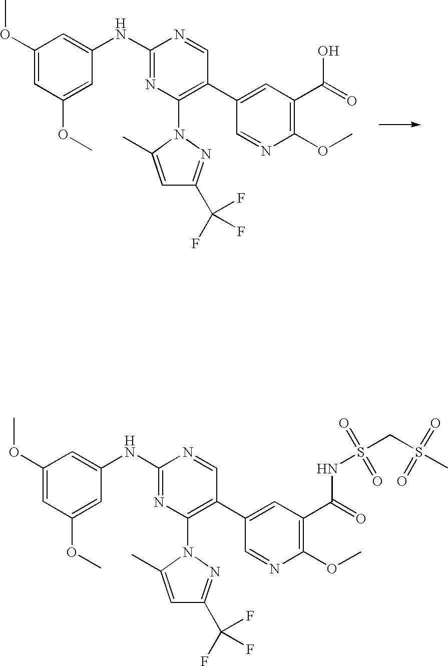

To a solution of 5-{2-[(3,5-dimethoxyphenyl)amino]-4-[5-methyl-3-(trifluoromethyl)-1H-pyrazol-1-yl]pyrimidin-5-yl}-2-methoxypyridine-3-carboxylic acid (Example 675, 0.23 mmol, 0.125 g) and 1-(methylsulfonyl)methanesulfonamide (Intermediate 340, 0.34 mmol, 0.06 g) in CH₂Cl₂ (10 mL), were added triethylamine (0.6 mmol, 0.1 mL), 2-chloro-1-methylpyridinium iodide (0.27 mmol, 0.07 g) and 4-(Dimethylamino)pyridine (0.04 mmol, 5 mg) and stirred at RT for 2 h. The reaction mixture was diluted with dichloromethane and further washed with 10% citric acid solution, water and brine. The organic layer was dried over Na₂SO₄ and concentrated. The crude mass was purified by 60-120 mesh silica (product eluted with 2% MeOH in CHCl₃) to afford 70 mg of the title compound.

| | | Mass spectrum and ¹H | |
|---|---|---|---|
| Compound | Structure | NMR | SM |
| Example 838 | 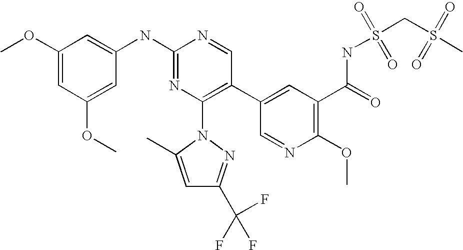 5-{2-[(3,5- dimethoxyphenyl) amino]-4-[5-methyl-3- (trifluoromethyl)-1H- pyrazol-1- yl]pyrimidin-5-yl}-2- methoxy-N- {[(methylsulfonyl) methyl]sulfonyl} pyridine-3-carboxamide | MS(ES): 684 (M − 1) for C₂₆H₂₆F₃N₇O₈S₂. 400 MHz, DMSO-d₆: δ 2.31 (s, 3H), 3.24 (s, 3H), 3.72 (s, 6H), 3.91 (s, 3H), 5.44 (s, 2H), 6.21 (t, J = 2.00 Hz, 1H), 6.75 (s, 1H), 7.05 (d, J = 1.88 Hz, 2H), 7.72 (d, J = 2.40 Hz, 1H), 7.95 (d, J = 2.28 Hz, 1H), 8.90 (s, 1H), 10.22 (s, 1H). | Intermediate 340 1- (methylsulfonyl) methanesulfonamide and Example 675 5-{2-[(3,5- dimethoxyphenyl) amino]-4-[5- methyl-3- (trifluoromethyl)- 1H-pyrazol-1- yl]pyrimidin-5- yl}-2- methoxypyridine- 3-carboxylic acid |

### Example 839

### 5-{2-[(3,5-dimethoxyphenyl)amino]-4-[5-methyl-3-(trifluoromethyl)-1H-pyrazol-1-yl]pyrimidin-5-yl}-2-methoxy-N-[1-methyl-1H-imidazol-4-yl)sulfonyl]pyridine-3-carboxamide

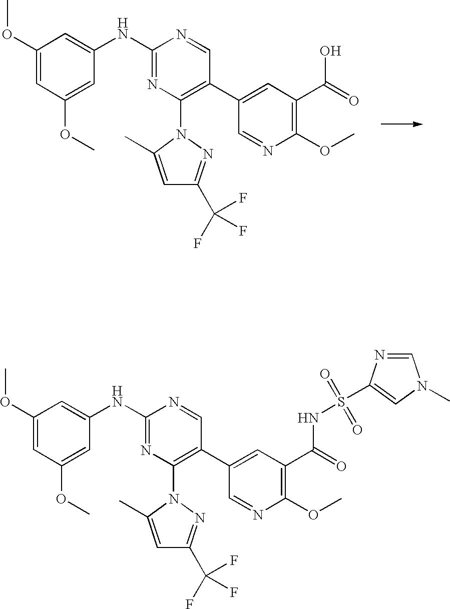

To a solution of 5-{2-[(3,5-dimethoxyphenyl)amino]-4-[5-methyl-3-(trifluoromethyl)-1H-pyrazol-1-yl]pyrimidin-5-yl}-2-methoxypyridine-3-carboxylic acid (Example 675, 0.28 mmol, 0.15 g) and 1-methyl-1H-imidazole-4-sulfonamide (Intermediate 341, 0.84 mmol, 0.14 g) in CH₂Cl₂ (10 mL), were added triethylamine (1.69 mmol, 0.23 mL), 2-chloro-1-methylpyridinium iodide (0.35 mmol, 0.09 g) and 4-(Dimethylamino)pyridine (0.056 mmol, 7 mg) and stirred at RT for 2 h. The reaction mixture was diluted with dichloromethane and further washed with 5% citric acid solution, water and brine. The organic layer was dried over Na₂SO₄ and concentrated. The crude mass was purified by 60-120 mesh silica (product eluted with 2% MeOH in CHCl₃) to afford 150 mg of the title compound.

| | | Mass spectrum and ¹H | |
|---|---|---|---|
| Compound | Structure | NMR | SM |
| Example 839 | 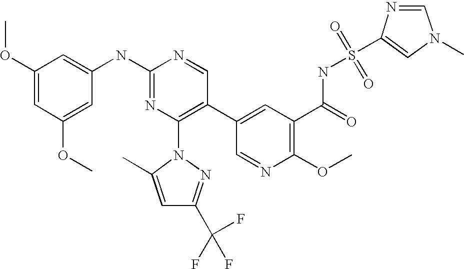 5-{2-[(3,5- dimethoxyphenyl)- amino]-4-[5-methyl-3- (trifluoromethyl)-1H- pyrazol-1- yl]pyrimidin-5-yl}-2- methoxy-N-[(1-methyl- 1H-imidazol-4- yl)sulfonyl]pyridine-3- carboxamide | MS(ES): 674 (M + 1) for C₂₈H₂₆F₃N₉O₆S. 400 MHz, DMSO-d₆: δ 2.29 (s, 3H), 3.71 (s, 3H), 3.73 (s, 6H), 3.88 (s, 3H), 6.20 (d, J = 2.04 Hz, 1H), 6.73 (s, 1H), 7.04 (d, J = 1.88 Hz, 2H), 7.60 (d, J = 2.32 Hz, 1H), 7.81 (s, 1H), 7.90 (s, 1H), 8.01 (s, 1H), 8.90 (s, 1H), 10.21 (s, 1H), 11.95 (s, 1H). | Intermediate 341 1-methyl- 1H- imidazole- 4- sulfonamide |

### Example 840

### 5-{2-[(3,5-dimethoxyphenyl)amino]-4-[5-methyl-3-(trifluoromethyl)-1H-pyrazol-1-yl]pyrimidin-5-yl}-N-[(1,1-dioxido-2,5-dihydrothiophen-3-yl)sulfonyl]-2-methoxypyridine-3-carboxamide

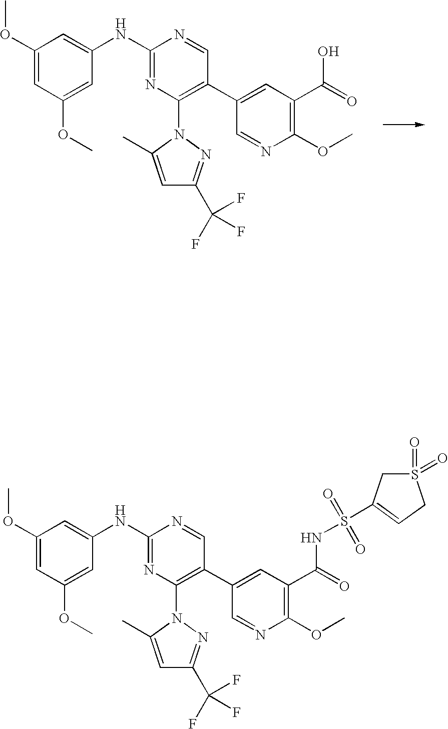

To a solution of 5-{2-[(3,5-dimethoxyphenyl)amino]-4-[5-methyl-3-(trifluoromethyl)-1H-pyrazol-1-yl]pyrimidin-5-yl}-2-methoxypyridine-3-carboxylic acid (Example 675, 0.56 mmol, 0.3 g) and 2,5-dihydrothiophene-3-sulfonamide 1,1-dioxide (Intermediate 343, 0.85 mmol, 0.17 g) in DMSO (10 mL), were added triethylamine (1.68 mmol, 0.23 mL), 2-chloro-1-methylpyridinium iodide (0.7 mmol, 0.18 g) and 4-(Dimethylamino)pyridine (0.11 mmol, 14 mg) and stirred at RT for 1 h. The reaction mixture was diluted with water and extracted into ethyl acetate. The organic layer was further washed with 10% citric acid solution, water and brine, dried over Na₂SO₄ and concentrated. The crude mass was purified by 60-120 mesh silica (product eluted with 2% MeOH in CHCl₃) to afford 200 mg of the title compound as a 7:3 mixture of two isomers.

| | | Mass spectrum and ¹H | |
|---|---|---|---|
| Compound | Structure | NMR | SM |
| Example 840 | 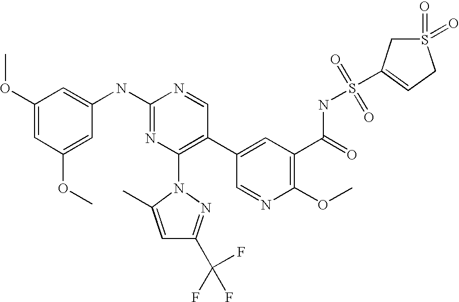 5-{2-[(3,5- dimethoxyphenyl) amino]-4-[5-methyl-3- (trifluoromethyl)-1H- pyrazol-1- yl]pyrimidin-5-yl}-N- [(1,1-dioxido-2,5- dihydrothiophen-3- yl)sulfonyl]-2- methoxypyridine-3- carboxamide | MS(ES): 710 (M + 1) for C₂₈H₂₆F₃N₇O₈S₂. 400 MHz, DMSO-d₆: δ 2.33 (s, 3H), 3.73 (s, 6H), 3.93 (m, 3H), 4.20 (br s, 2H), 4.34 (br s, 2H), 6.22 (s, 1H), 6.76- 6.78 (m, 1H), 7.05 (s, 2H), 7.16 (br s, 1H), 7.73 (d, J = 2.32 Hz, 1H), 7.93-7.96 (m, 1H), 8.93 (d, J = 2.04 Hz, 1H), 10.23 (s, 1H), 12.37 (br s, 1H). | Intermediate 343 2,5- dihydrothiophene- 3- sulfonamide 1,1-dioxide |

### Example 841

### 5-{2-[(3,5-dimethoxyphenyl)amino]-4-[5-methyl-3-(trifluoromethyl)-1H-pyrazol-1-yl]pyrimidin-5-yl}-2-methoxy-N-[(6-methyl-2,4-dioxo-1,2,3,4-tetrahydropyrimidin-5-yl)sulfonyl]pyridine-3-carboxamide

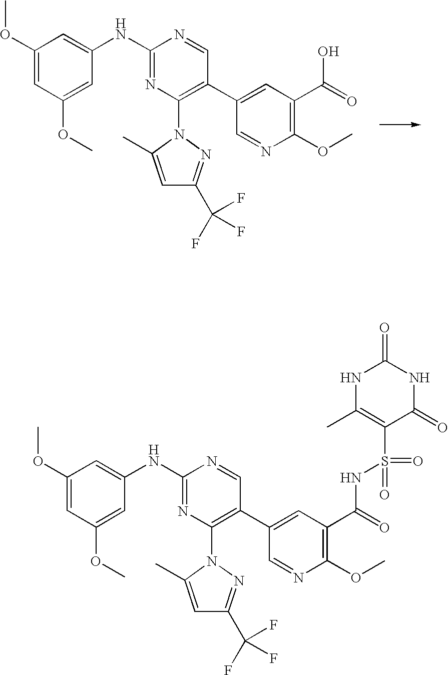

To a solution of 5-{2-[(3,5-dimethoxyphenyl)amino]-4-[5-methyl-3-(trifluoromethyl)-1H-pyrazol-1-yl]pyrimidin-5-yl}-2-methoxypyridine-3-carboxylic acid (Example 675, 0.28 mmol, 0.15 g) and 6-methyl-2,4-dioxo-1,2,3,4-tetrahydropyrimidine-5-sulfonamide (Intermediate 345, 0.42 mmol, 0.09 g) in DMSO (6 mL), were added triethylamine (1.4 mmol, 0.2 mL), 2-chloro-1-methylpyridinium iodide (0.42 mmol, 0.11 g) and 4-(Dimethylamino)pyridine (0.08 mmol, 10 mg) and stirred at RT for 1 h. The reaction mixture was diluted with water and extracted into ethyl acetate. The organic layer was further washed with 10% citric acid solution, water and brine, dried over Na₂SO₄ and concentrated in vacuo. The crude mass was purified by 60-120 mesh silica (product eluted with 2% MeOH in CHCl₃) to afford 70 mg of the title compound.

| | | Mass spectrum and | |
|---|---|---|---|
| Compound | Structure | ¹H NMR/ SM | |
| Example 841 | 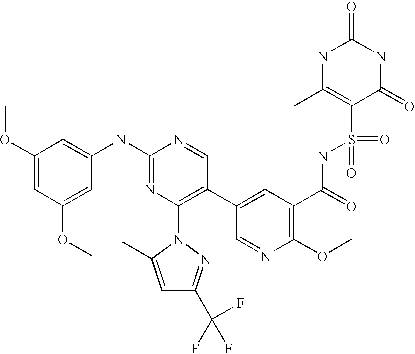 {2[(3,5- dimethoxyphenyl)amino]- 4-[5-methyl-3- (trifluoromethyl)-1H- pyrazol-1-yl]pyrimidin-5- yl}-2-methoxy-N-[(6- methyl-2,4-dioxo-1,2,3,4- tetrahydropyrimidin-5- yl)sulfonyl]pyridine-3- carboxamide | MS(ES): 718 (M + 1) for C₂₉H₂₆F₃N₉O₈S. 400 MHz, DMSO-d₆: δ 2.29 (s, 3H), 2.52 (s, 3H), 3.71 (s, 6H), 3.87 (s, 3H), 6.20 (s, 1H), 6.73 (s, 1H), 7.04 (s, 2H), 7.64 (s, 1H), 7.85 (br s, 1H), 8.90 (s, 1H), 10.21 (s, 1H), 11.53 (br s, 1H), 11.97 (br s, 1H). | Intermediate 345 6-methyl-2,4- dioxo-1,2,3,4- tetrahydro- pyrimidine-5- sulfonamide |

### Example 842

### 5-{2-[(3,5-dimethoxyphenyl)amino]-4-[5-methyl-3-(trifluoromethyl)-1H-pyrazol-1-yl]pyrimidin-5-yl}-2-methoxy-N-[(1,3,5-trimethyl-1H-pyrazol-4-yl)sulfonyl]pyridine-3-carboxamide

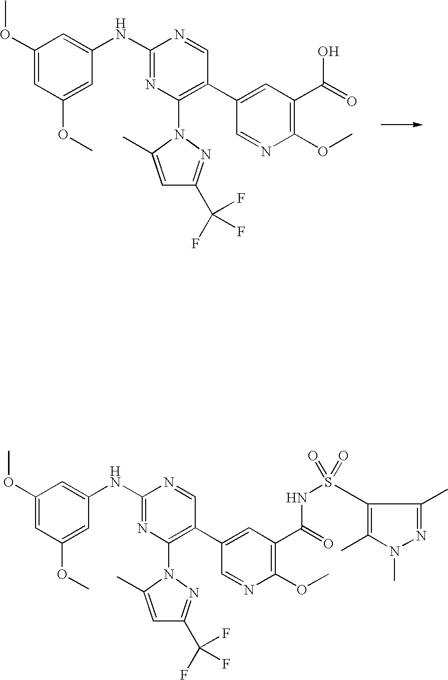

To a solution of 5-{2-[(3,5-dimethoxyphenyl)amino]-4-[5-methyl-3-(trifluoromethyl)-1H-pyrazol-1-yl]pyrimidin-5-yl}-2-methoxypyridine-3-carboxylic acid (Example 675, 0.31 mmol, 0.165 g) and 1,3,5-trimethyl-1H-pyrazole-4-sulfonamide (Intermediate 346, 0.78 mmol, 0.147 g) in CH₂Cl₂ (15 mL), were added triethylamine (0.933 mmol, 0.1302 mL), 2-chloro-1-methylpyridinium iodide (0.38 mmol, 99 mg) and 4-(Dimethylamino)pyridine (0.062 mmol, 8 mg) and stirred at RT for 90 min. The reaction mixture was diluted with dichloromethane (30 mL) and further washed with 25% citric acid solution (2×25 mL), water (50 mL) and brine (25 mL). The organic layer was dried over Na₂SO₄ and concentrated. The crude mass was purified by 60-120 mesh silica (product eluted with 1-2% MeOH in CHCl₃) to afford 60 mg of the title compound as a white solid.

| | | Mass spectrum and ¹H | |
|---|---|---|---|
| Compound | Structure | NMR | SM |
| Example 842 | 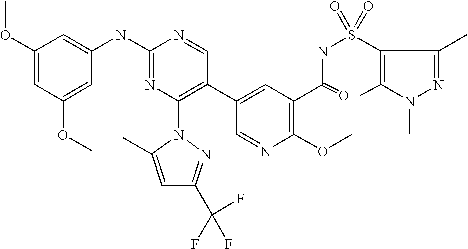 5-{2-[(3,5- dimethoxyphenyl) amino]-4-[5-methyl-3- (trifluoromethyl)-1H- pyrazol-1- yl]pyrimidin-5-yl}-2- methoxy-N-[(1,3,5- 4-yl)sulfonyl]pyridine- 3-carboxamide | MS (ES): 702 (M + 1) for C₃₀H₃₀F₃N₉O₆S. 400 MHz, DMSO-d₆: δ 2.29 (s, 3H), 2.31 (s, 3H), 2.47 (s, 3H), 3.74 (s, 9H), 3.89 (s, 3H), 6.21 (t, J = 2.12 Hz, 1H), 6.74 (s, 1H), 7.04 (d, J = 2.12 Hz, 2H), 7.61 (d, J = 2.44 Hz, 1H), 7.92 (d, J = 2.40 Hz, 1H), 8.91 (s, 1H), 10.21 (s, 1H), 12.04 (s, 1H). | Intermediate 346 1,3,5- trimethyl- 1H- pyrazole-4- sulfonamide |

### Example 843

### 5-{2-[(3,5-dimethoxyphenyl)amino]-4-[5-methyl-3-(trifluoromethyl)-1H-pyrazol-1-yl]pyrimidin-5-yl}-2-methoxy-N-[(3-methyl-2-oxo-2,3-dihydro-1,3-benzoxazol-6-yl)sulfonyl]pyridine-3-carboxamide

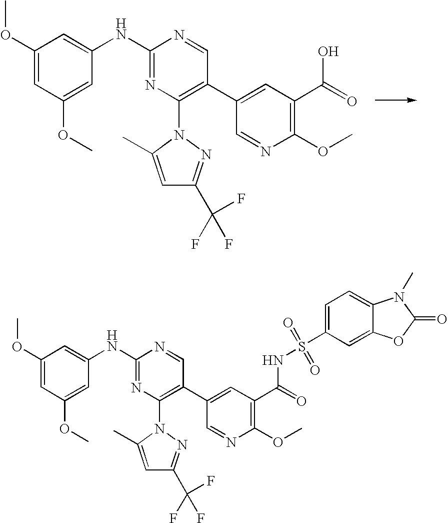

To a solution of 5-{2-[(3,5-dimethoxyphenyl)amino]-4-[5-methyl-3-(trifluoromethyl)-1H-pyrazol-1-yl]pyrimidin-5-yl}-2-methoxypyridine-3-carboxylic acid (Example 675, 0.38 mmol, 0.2 g) and 3-methyl-2-oxo-2,3-dihydro-1,3-benzoxazole-6-sulfonamide (Intermediate 347, 0.46 mmol, 0.1 g) in CH₂Cl₂ (25 mL) were added triethylamine (1.14 mmol, 0.16 mL), 2-chloro-1-methylpyridinium iodide (0.46 mmol, 0.116 g) and 4-(Dimethylamino)pyridine (0.076 mmol, 10 mg) and stirred for 90 min at RT. The reaction mixture was diluted with dichloromethane (50 mL) and further washed with 25% citric acid solution (2×50 mL), water (100 mL) and brine (75 mL). The organic layer was dried over Na₂SO₄ and concentrated. The crude mass was purified by 60-120 mesh silica (product eluted with 1-1.5% methanol/chloroform) to afford 200 mg of white solid Example 843.

| Com- | | Mass spectrum and ¹H | |
|---|---|---|---|
| pound | Structure | NMR | SM |
| Example 843 | 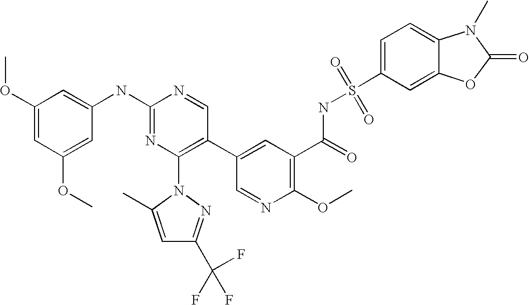 5-{2-[(3,5- dimethoxyphenyl) amino]-4-[5-methyl-3- (trifluoromethyl)-1H- pyrazol-1- yl]pyrimidin-5-yl}-2- methoxy-N-[(3-methyl- 2-oxo-2,3-dihydro-1,3- benzoxazol-6- yl)sulfonyl]pyridine-3- carboxamide | MS(ES): 741 (M + 1) for C₃₂H₂₇F₃N₈O₈S. 400 MHz, DMSO-d₆: δ 2.30 (s, 3H), 3.40 (s, 3H), 3.72 (s, 6H), 3.91 (s, 3H), 6.21 (t, J = 2.08 Hz, 1H), 6.70 (s, 1H), 7.04 (d, J = 2.04 Hz, 2H), 7.51 (d, J = 8.36 Hz, 1H), 7.57 (d, J = 2.40 Hz, 1H), 7.85 (d, J = 1.52 Hz, 1H), 7.89-7.91 (m, 1H), 7.97 (d, J = 2.40 Hz, 1H), 8.89 (s, 1H), 10.20 (s, 1H), 12.17 (s, 1H). | Intermediate 347 3-methyl-2- oxo-2,3- dihydro-1,3- benzoxazole- 6- sulfonamide |

### Example 844

### N-[(3-acetylphenyl)sulfonyl]-5-{2-[(3,5-dimethoxyphenyl)amino]-4-[5-methyl-3-(trifluoromethyl)-1H-pyrazol-1-yl]pyrimidin-5-yl}-2-methoxypyridine-3-carboxamide

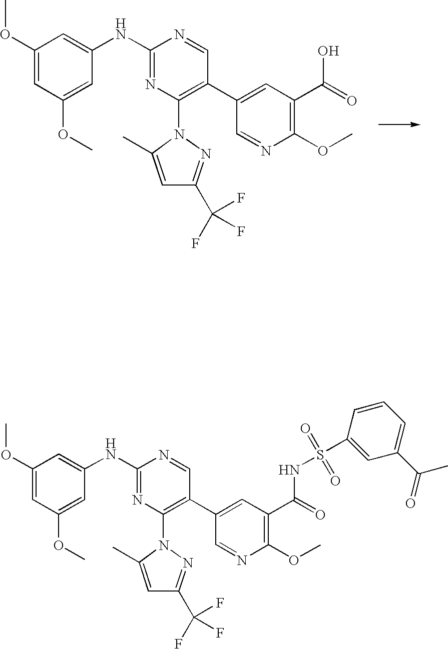

To a solution of 5-{2-[(3,5-dimethoxyphenyl)amino]-4-[5-methyl-3-(trifluoromethyl)-1H-pyrazol-1-yl]pyrimidin-5-yl}-2-methoxypyridine-3-carboxylic acid (Example 675, 0.19 mmol, 0.100 g) and 3-acetylbenzenesulfonamide (Intermediate 348, 0.37 mmol, 75 mg) in CH₂Cl₂ (10 mL), were added triethylamine (0.57 mmol, 0.08 mL), 2-chloro-1-methylpyridinium iodide (0.23 mmol, 60 mg) and 4-(Dimethylamino)pyridine (0.03 mmol, 5 mg) and stirred for 1 h at RT. The reaction mixture was diluted with dichloromethane (50 mL) and further washed with water (100 mL) and brine (75 mL). The organic layer was dried over Na₂SO₄ and concentrated in vacuo. The crude mass was purified by 60-120 mesh silica (product eluted with 1.5% methanol/chloroform) to afford 60 mg of the title compound as a white solid.

| | | Mass spectrum and ¹H | |
|---|---|---|---|
| Compound | Structure | NMR | SM |
| Example 844 | 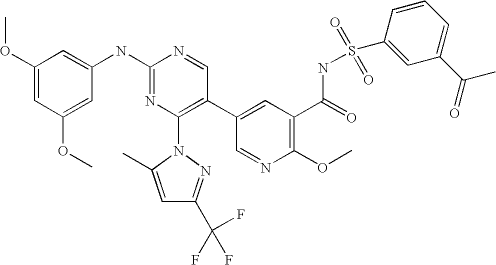 N-[(3- acetylphenyl)sulfonyl]- 5-{2-[(3,5- dimethoxyphenyl) amino]-4-[5-methyl-3- (trifluoromethyl)-1H- pyrazol-1- yl]pyrimidin-5-yl}-2- methoxypyridine-3- carboxamide | MS(ES): 712 (M + 1) for C₃₂H₂₈F₃N₇O₇S. 400 MHz, DMSO-d₆: δ 2.30 (s, 3H), 2.67 (s, 3H), 3.72 (s, 6H), 3.90 (s, 3H), 6.21 (t, J = 2.12 Hz, 1H), 6.69 (s, 1H), 7.04 (d, J = 2.12 Hz, 2H), 7.56 (d, J = 2.44 Hz, 1H), 7.84 (t, J = 7.84 Hz, 1H), 7.98 (d, J = 2.40 Hz, 1H), 8.22 (d, J = 8.44 Hz, 1H), 8.34 (d, J = 7.88 Hz, 1H), 8.46 (d, J = 1.64 Hz, 1H), 8.89 (s, 1H), 10.20 (s, 1H), 12.36 (br s, 1H). | Intermediate 348 3- acetylbenzene- sulfonamide |

### Example 845

### 5-{2-[(3,5-dimethoxyphenyl)amino]-4-[5-methyl-3-(trifluoromethyl)-1H-pyrazol-1-yl]pyrimidin-5-yl}-2-methoxy-N-{[1-methyl-3-(trifluoromethyl)-1H-pyrazol-4-yl]sulfonyl}pyridine-3-carboxamide

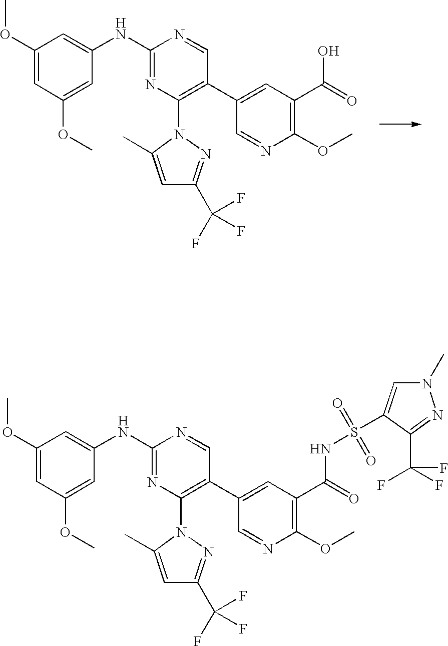

To a solution of 5-{2-[(3,5-dimethoxyphenyl)amino]-4-[5-methyl-3-(trifluoromethyl)-1H-pyrazol-1-yl]pyrimidin-5-yl}-2-methoxypyridine-3-carboxylic acid (Example 675, 0.19 mmol, 0.100 g) and 1-methyl-3-(trifluoromethyl)-1H-pyrazole-4-sulfonamide (Intermediate 349, 0.38 mmol, 0.087 g) in CH₂Cl₂ (25 mL), were added 2-chloro-1-methylpyridinium iodide (0.23 mmol, 58 mg), triethylamine (0.57 mmol, 0.08 mL) and 4-(Dimethylamino)pyridine (0.038 mmol, 5 mg) and stirred for 4-5 h at RT. The reaction mixture was diluted with dichloromethane (50 mL) and further washed with 25% citric acid solution (2×50 mL), water (100 mL) and brine (75 mL). The organic layer was dried over Na₂SO₄ and concentrated. The crude mass was purified by 60-120 mesh silica (product eluted with 4-5% methanol/chloroform) to afford 110 mg of white solid in 78% purity by LCMS. This was further purified using RP-HPLC (Kromasil C18 column (50×250 mm, 10 μm); using a binary solvent mixture of 0.1% TFA in water(A)/MeOH (B) (0-20 min: 10-70% B, 20-30 min: 70-80% B and 30-40 min: 80% B; 40-45 min: 80-100% B, 45-50 min: 100% B, 50-52 min: 100-10 B, flow rate of 40 mL/min; Separation was monitored at 210 and 300 nm) to give 63 mg of Example 845.

| | | Mass spectrum and ¹H | |
|---|---|---|---|
| Compound | Structure | NMR | SM |
| Example 845 | 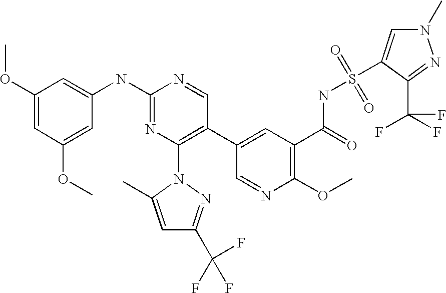 5-{2-[(3,5- dimethoxyphenyl) amino]-4-[5-methyl-3- (trifluoromethyl)-1H- pyrazol-1- yl]pyrimidin-5-yl}-2- methoxy-N-{[1- methyl-3- (trifluoromethyl)-1H- pyrazol-4- yl]sulfonyl}pyridine-3- carboxamide | MS(ES): 742 (M + 1) for C₂₉H₂₅F₆N₉O₆S. 400 MHz, DMSO-d₆: δ 2.29 (s, 3H), 3.71 (s, 6H), 3.87 (s, 3H), 3.99 (s, 3H), 6.20 (t, J = 2.08 Hz, 1H), 6.70 (s, 1H), 7.03 (d, J = 2.00 Hz, 2H), 7.54 (d, J = 2.40 Hz, 1H), 7.98 (d, J = 2.28 Hz, 1H), 8.75 (s, 1H), 8.90 (s, 1H), 10.21 (s, 1H), 12.45 (s, 1H). | Intermediate 349 1-methyl-3- (trifluoro- methyl)-1H- pyrazol-4- sulfonamide |

### Example 846

### N-({4-[acetylamino)methyl]phenyl}sulfonyl)-5-{2-[(3,5-dimethoxyphenyl)amino]-4-[5-methyl-3-(trifluoromethyl)-1H-pyrazol-1-yl]pyrimidin-5-yl}-2-methoxypyridine-3-carboxamide

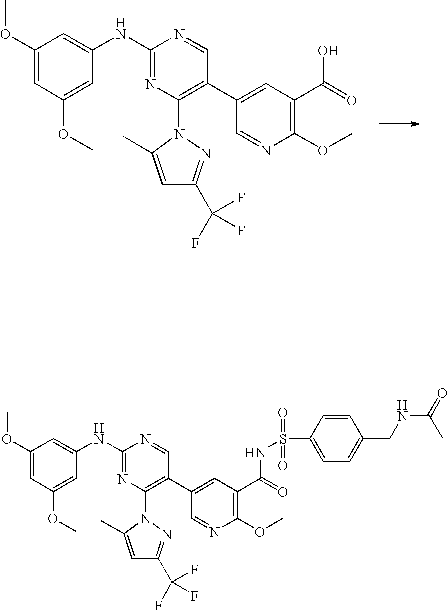

To a solution of 5-{2-[(3,5-dimethoxyphenyl)amino]-4-[5-methyl-3-(trifluoromethyl)-1H-pyrazol-1-yl]pyrimidin-5-yl}-2-methoxypyridine-3-carboxylic acid (Example 675, 0.19 mmol, 0.100 g) and N-(4-sulfamoylbenzyl)acetamide (Intermediate 351 0.23 mmol, 52 mg) in CH₂Cl₂ (25 mL) were added triethylamine (0.57 mmol, 0.08 mL), 2-chloro-1-methylpyridinium iodide (0.23 mmol, 58 mg) and 4-(Dimethylamino)pyridine (0.038 mmol, 5 mg) and stirred for 4-5 h at RT. The reaction mixture was diluted with dichloromethane (50 mL) and further washed with 25% citric acid solution (2×50 mL), water (100 mL) and brine (75 mL). The organic layer was dried over Na₂SO₄ and concentrated. The crude mass was purified by RP-HPLC (Kromasil C18 column (50×250 mm, 10 μm); using a binary solvent mixture of 0.1% HCOOH in water(A)/MeOH (B) (0-20 min: 20-70% B, 20-30 min: 70-80% B and 30-45 min: 80-100% B; 45-55 min: 100% B, 55-57 min: 100-20% B, flow rate of 40 mL/min; Separation was monitored at 210, 280 and 320 nm) to give 62 mg of Example 846.

| | | Mass spectrum and ¹H | |
|---|---|---|---|
| Compound | Structure | NMR | SM |
| Example 846 | 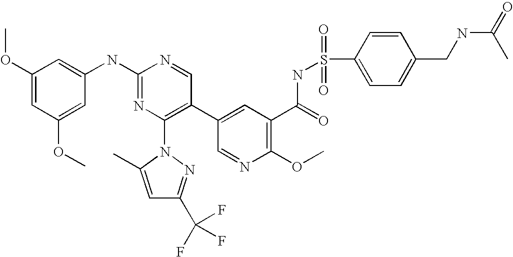 N-({4-[(acetylamino)methyl]phenyl}sulfonyl)-5-{2-[(3,5- dimethoxyphenyl)amino]-4-[5-methyl-3-(trifluoromethyl)-1H- pyrazol-1-yl]pyrimidin-5-yl}-2-methoxypyridine-3-carboxamide | MS(ES): 741 (M + 1) for C₃₃H₃₁F₃N₈O₇S. 400 MHz, DMSO-d₆: δ 1.90 (s, 3H), 2.31 (s, 3H), 3.72 (s, 6H), 3.90 (s, 3H), 4.35 (d, J = 5.96 Hz, 2H), 6.21 (t, J = 2.00 Hz, 1H), 6.72 (s, 1H), 7.04 (d, J = 1.80 Hz, 2H), 7.50 (d, J = 8.20 Hz, 2H), 7.60 (d, J = 2.20 Hz, 1H), 7.91-7.93 (m, 3H), 8.49 (t, J = 5.80 Hz, 1H), 8.90 (s, 1H), 10.20 (s, 1H), 12.18 (s, 1H). | Intermediate 51 N-(4-sulfamoyl- benzyl)acetamide |

### Example 847

### 5-{2-[(3,5-dimethoxyphenyl)amino]-4-[5-methyl-3-(trifluoromethyl)-1H-pyrazol-1-yl]pyrimidin-5-yl}-N-[(1,3-dimethyl-2,4-dioxo-1,2,3,4-tetrahydropyrimidin-5-yl)sulfonyl]-2-methoxypyridine-3-carboxamide

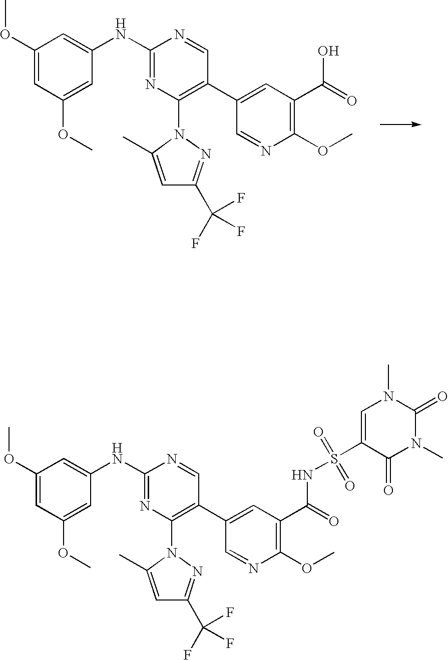

To a solution of 5-{2-[(3,5-dimethoxyphenyl)amino]-4-[5-methyl-3-(trifluoromethyl)-1H-pyrazol-1-yl]pyrimidin-5-yl}-2-methoxypyridine-3-carboxylic acid (Example 675, 0.19 mmol, 0.100 g) and 1,3-dimethyl-2,4-dioxo-1,2,3,4-tetrahydropyrimidine-5-sulfonamide (Intermediate 352 0.23 mmol, 48 mg) in CH₂Cl₂ (25 mL) were added triethylamine (0.57 mmol, 0.08 mL), 2-chloro-1-methylpyridinium iodide (0.23 mmol, 58 mg) and 4-(Dimethylamino)pyridine (0.038 mmol, 5 mg) and stirred 4-5 h at RT. The reaction mixture was diluted with dichloromethane (50 mL) and further washed with 25% citric acid solution (2×50 mL), water (100 mL) and brine (75 mL). The organic layer was dried over Na₂SO₄ and concentrated. The crude mass was purified by RP-HPLC (Kromasil C18 column (50×250 mm, 10 μm); using a binary solvent mixture of 0.1% HCOOH in water(A)/MeOH (B) (0-20 min: 20-70% B, 20-30 min: 70-80% B and 30-45 min: 80-100% B; 45-55 min: 100% B, 55-57 min: 100-20% B, flow rate of 40 mL/min; Separation was monitored at 210, 280 and 320 nm) to give 55 mg of Example 847.

| | | | |
|---|---|---|---|
| | | Mass spectrum and ¹H | |
| Compound | Structure | NMR | SM |

| | | | |
|---|---|---|---|
| Example 847 | 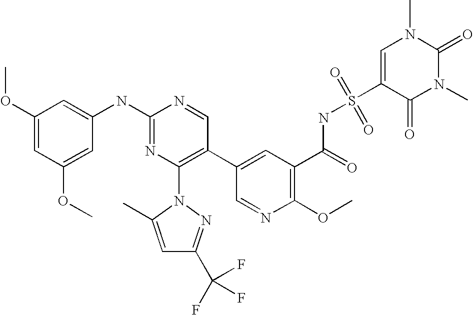 5-{2-[(34,5-dimethoxyphenyl)amino]-4-[5-methyl-3- (trifluoromethyl)-1H-pyrazol-1-yl]pyrimidin-5-yl}-N- [(1,3-dimethyl-2,4-dioxo-1,2,3,4-tetrahydropyrimidin-5- yl)sulfonyl]-2-methoxypyridine-3-carboxamide | MS(ES): 730 (M − 1) for C₃₀H₂₈F₃N₉O₈S. 400 MHz, DMSO-d₆: δ 2.31 (s, 3H), 3.19 (s, 3H), 3.50 (s, 3H), 3.72 (s, 6H), 3.90 (s, 3H), 6.22 (t, J = 2.12 Hz, 1H), 6.73 (s, 1H), 7.05 (d, J = 2.00 Hz, 2H), 7.64 (d, J = 2.40 Hz, 1H), 7.94 (d, J = 2.32 Hz, 1H), 8.76 (s, 1H), 8.93 (s, 1H), 10.24 (s, 1H). | Intermediate 352 1,3-dimethyl- 2,4-dioxo-1,2,3,4- tetrahydropyrimidine- 5-sulfonamide |

### Example 848

### 5-{2-[(3,5-dimethoxyphenyl)amino]-4-[5-methyl-3-(trifluoromethyl)-1H-pyrazol-1-yl]pyrimidin-5-yl}-N-{[2-(2,5-dioxopyrrolidin-1-yl)ethyl]sulfonyl}-2-methoxypyridine-3-carboxamide

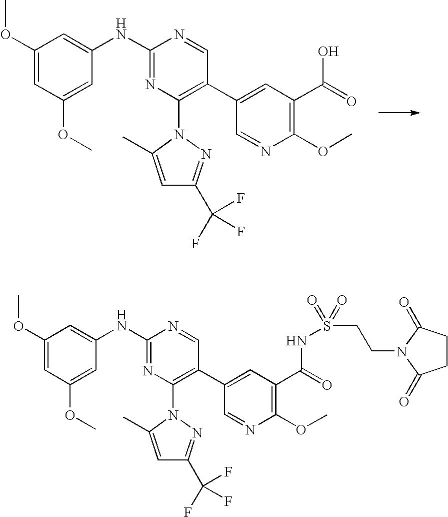

To a solution of 5-{2-[(3,5-dimethoxyphenyl)amino]-4-[5-methyl-3-(trifluoromethyl)-1H-pyrazol-1-yl]pyrimidin-5-yl}-2-methoxypyridine-3-carboxylic acid (Example 675, 0.36 mmol, 0.19 g) and 2-(2,5-dioxopyrrolidin-1-yl)ethanesulfonamide (Intermediate 353, 0.9 mmol, 0.19 g) in DMSO (10 mL), were added triethylamine (1.07 mmol, 0.15 mL), 2-chloro-1-methylpyridinium iodide (0.45 mmol, 0.12 g) and 4-(Dimethylamino)pyridine (0.07 mmol, 10 mg) and stirred at RT for 1 h. The reaction mixture was diluted with water and extracted into ethyl acetate. The organic layer was further washed with 10% citric acid solution, water and brine, dried over Na₂SO₄ and concentrated. The crude mass was purified by 60-120 mesh silica (product eluted with 2% MeOH in CHCl₃) followed by washing with water and 1.5 N HCl to afford 58 mg of the title compound (0.08 mmol, 22%).

| | | | |
|---|---|---|---|
| | | Mass spectrum and ¹H | |
| Compound | Structure | NMR | SM |

| | | | |
|---|---|---|---|
| Example 848 | 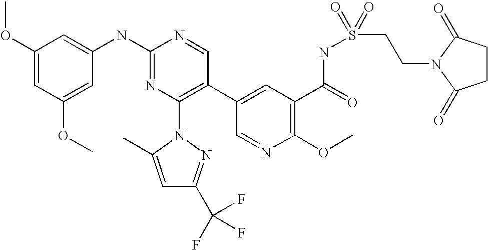 55-{2-[(3,5-dimethoxyphenyl)-amino]-4-[5-methyl-3- (trifluoromethyl)-1H-pyrazol-1-yl]pyrimidin-5-yl}-N-{[2-(2,5- dioxopyrrolidin-1-yl)ethyl]sulfonyl}-2-methoxypyridine-3- carboxamide | MS(ES):719 (M + 1) for C₃₀H₂₉F₃N₈O₈S. 400 MHz, DMSO-d₆: δ 2.34 (s, 3H), 2.60 (s, 3H), 3.68- 3.70 (m, 2H), 3.73 (s, 6H), 3.78 (t, J = 4.72 Hz, 2H), 3.94 (s, 3H), 6.22 (s, 1H), 6.77 (s, 1H), 7.05 (d, J = 2.00 Hz, 2H), 7.75 (d, J = 2.40 Hz, 1H), 7.97 (d, J = 2.04 Hz, 1H), 8.93 (s, 1H), 10.23 (s, 1H), 11.93 (br s, 1H). | Intermediate 353 2-(2,5- dioxopyrrolidin-1- yl)ethanesulfonamide |

### Example 849

### 5-{2-[(3,5-dimethoxyphenyl)amino]-4-[5-methyl-3-(trifluoromethyl)-1H-pyrazol-1-yl]pyrimidin-5-yl}-2-methoxy-N-(1H-pyrazol-4-ylsulfonyl)pyridine-3-carboxamide

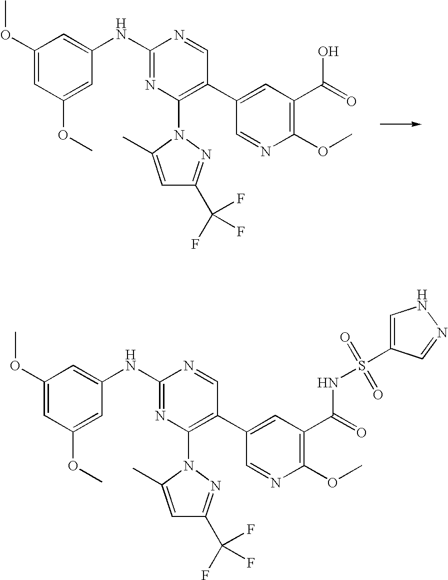

To a solution of 5-{2-[(3,5-dimethoxyphenyl)amino]-4-[5-methyl-3-(trifluoromethyl)-1H-pyrazol-1-yl]pyrimidin-5-yl}-2-methoxypyridine-3-carboxylic acid (Example 675, 0.38 mmol, 0.2 g) and 1H-pyrazole-4-sulfonamide (Intermediate 354, 0.45 mmol, 0.07 g) in CH₂Cl₂ (25 mL) were added triethylamine (1.14 mmol, 0.16 mL), 2-chloro-1-methylpyridinium iodide (0.46 mmol, 0.116 g) and 4-(Dimethylamino)pyridine (0.076 mmol, 10 mg) and stirred for 4-5 h at RT.

The reaction mixture was diluted with dichloromethane (50 mL) and further washed with 25% citric acid solution (2×50 mL), water (100 mL) and brine (75 mL). The organic layer was dried over Na₂SO₄ and concentrated. The crude mass was purified by 60-120 mesh silica (product eluted with 5-6% methanol/chloroform) to afford 36 mg of white solid Example 849.

| | | Mass spectrum and ¹H | |
|---|---|---|---|
| Compound | Structure | NMR | SM |
| Example 849 | 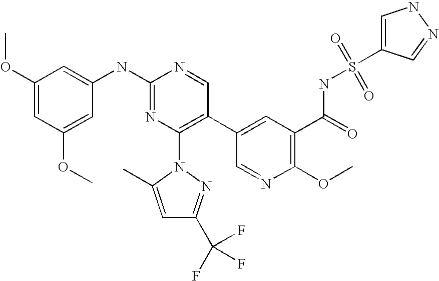 5-{2-[(3,5-dimethoxyphenyl)amino]-4-[5-methyl-3- (trifluoromethyl)-1H-pyrazol-1-yl]pyrimidin-5-yl}-2- methoxy-N-(1H-pyrazol-4-ylsulfonyl)pyridine-3- carboxamide | MS(ES): 660 (M + 1) for C₂₇H₂₄F₃N₉O₆S. 400 MHz, DMSO-d₆: δ 2.29 (s, 3H), 3.71 (s, 6H), 3.89 (s, 3H), 6.20 (t, J = 2.16 Hz, 1H), 6.71 (s, 1H), 7.03 (d, J = 2.12 Hz, 2H), 7.62 (d, J = 2.44 Hz, 1H), 7.93 (d, J = 2.24 Hz, 2H), 8.47 (s, 1H), 8.90 (s, 1H), 10.20 (s, 1H), 11.99 (s, 1H), 13.74 (s, 1H). | Intermediate 354 1H-pyrazole-4- sulfonamide |

### Example 850

### methyl 5-{2-[(3,5-dimethoxyphenyl)amino]-4-[3-(trifluoromethyl)-1H-pyrazol-1-yl]pyrimidin-5-yl}-2-(methylsulfanyl)pyridine-3-carboxylate

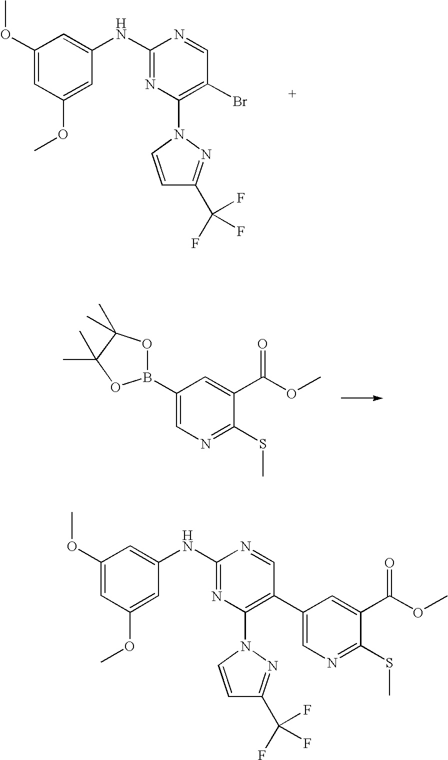

A suspension of 5-bromo-N-(3,5-dimethoxyphenyl)-4-[3-(trifluoromethyl)-1H-pyrazol-1-yl]pyrimidin-2-amine (Intermediate 215, 0.45 mmol, 0.2 g), a mixture of methyl 2-(methylsulfanyl)-5-(4,4,5,5-tetramethyl-1,3,2-dioxaborolan-2-yl)pyridine-3-carboxylate and [5-(methoxycarbonyl)-6-(methylsulfanyl)pyridin-3-yl]boronic acid (Intermediate 359, 0.58 mmol based on the boronic ester, 0.180 g), [1,1′-bis(diphenylphosphino)ferrocene]dichloropalladium(II) complex with dichloromethane (0.04 mmol, 0.0367 g) and sodium carbonate (0.45 mmol, 0.0477 g) in acetonitrile/water (5:1) was degassed and heated to 90° C. for 30 min under an inert atmosphere. The reaction mass was passed through a celite bed and solvent was concentrated in vacuo. The resultant residue taken in

EtOAc (50 mL) was washed with water, brine, dried over Na₂SO₄, filtered and concentrated. The crude mass was further purified by silica gel column chromatography (product eluted with 3% Et₃N EtOAc) to yield 0.22 g of the product.

| | | Mass spectrum and ¹H | |
|---|---|---|---|
| Compound | Structure | NMR | SM |
| Example 850 | 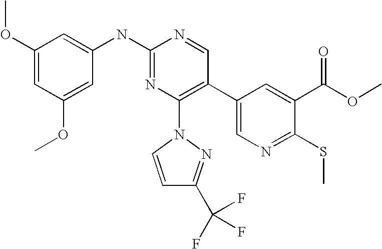 methyl 5-{2-[(3,5-dimethoxyphenyl)amino]-4-[3- (trifluoromethyl)-1H-pyrazol-1-yl]pyrimidin-5-yl}-2- (methylsulfanyl)pyrimidine-3-carboxylate | MS(ES): 547 (M + 1) for C₂₄H₂₁F₃N₆O₄S. 400 MHz, DMSO-d₆: δ 2.47 (s, 3H), 3.75 (s, 6H), 3.81 (s, 3H), 6.22 (t, J = 2.20 Hz, 1H), 7.06 (d, J = 2.52 Hz, 1H), 7.10 (d, J = 2.16 Hz, 2H), 7.94 (d, J = 2.32 Hz, 1H), 8.51 (d, J = 1.40 Hz, 1H), 8.58 (d, J = 2.32 Hz, 1H), 8.83 (s, 1H), 10.23 (s, 1H). | Intermediate 215 5-bromo-N-(3,5-dimethoxyphenyl)- 4-[3-(trifluoromethyl)-1H-pyrazol-1- yl]pyrimidin-2-amine and Intermediate 359 mixture of methyl 2-(methylsulfanyl)- 5-(4,4,5,5-tetramethyl-1,3,2-dioxaborolan- 2-yl)pyridine-3-carboxylate and [5- (methoxycarbonyl)-6-(methylsulfanyl) pyridin-3-yl]boronic acid |

### Example 851

### 5-{2-[(3,5-dimethoxyphenyl)amino]-4-[3-(trifluoromethyl)-1H-pyrazol-1-yl]pyrimidin-5-yl}-2-(methylsulfanyl)pyridine-3-carboxylic acid

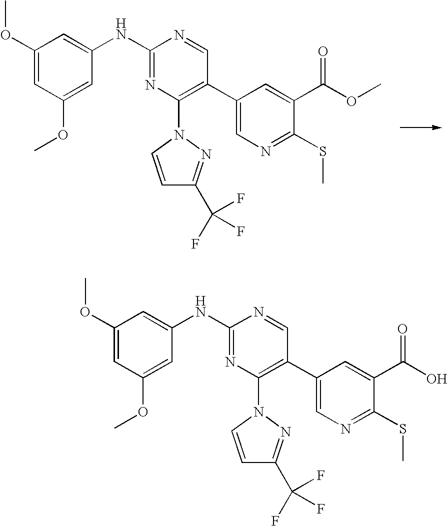

To 160 mg of methyl 5-{2-[(3,5-dimethoxyphenyl)amino]-4-[3-(trifluoromethyl)-1H-pyrazol-1-yl]pyrimidin-5-yl}-2-(methylsulfanyl)pyridine-3-carboxylate (Example 850, 0.29 mmol) taken in a mixture of THF (5 mL) and water (5 mL), was added Sodium hydroxide (0.73 mmol, 0.029 g) and stirred at room temperature for 3 h. The mixture was carefully acidified with 1 N HCl and the solid obtained was filtered, dried to yield the desired product (0.13 g).

| | | Mass spectrum and ¹H | |
|---|---|---|---|
| Compound | Structure | NMR | SM |
| Example 851 | 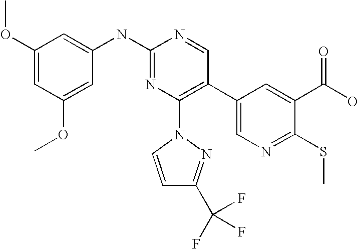 5-{2-[(3,5-dimethoxyphenyl)-amino]-4-[3- (trifluoromethyl)-1H-pyrazol-1-yl]pyrimidin-5-yl}-2- (methylsulfanyl)pyrimidine- 3-carboxylic acid | MS(ES): 533 (M + 1) for C₂₃H₁₉F₃N₆O₄S. 400 MHz, DMSO-d₆: δ 2.44 (s, 3H), 3.75 (s, 6H), 6.22 (t, J = 2.12 Hz, 1H), 7.05 (d, J = 2.60 Hz, 1H), 7.11 (d, J = 2.12 Hz, 2H), 7.91 (d, J = 2.32 Hz, 1H), 8.49 (d, J = 1.60 Hz, 1H), 8.54 (d, J = 2.16 Hz, 1H), 8.82 (s, 1H), 10.22 (s, 1H), 13.44 (s, 1H). | Example 850 methyl 5-{2-[(3,5- dimethoxyphenyl)amino]- 4-[3-(trifluoromethyl)-1H- pyrazol-1-yl]pyrimidin- 5-yl}-2- (methylsulfanyl)pyridine- 3-carboxylate |

### Example 852

### 5-{2-[(3,5-dimethoxyphenyl)amino]-4-[3-(trifluoromethyl)-1H-pyrazol-1-yl]pyrimidin-5-yl}-2-(methylsulfanyl)-N-(methylsulfonyl)pyridine-3-carboxamide

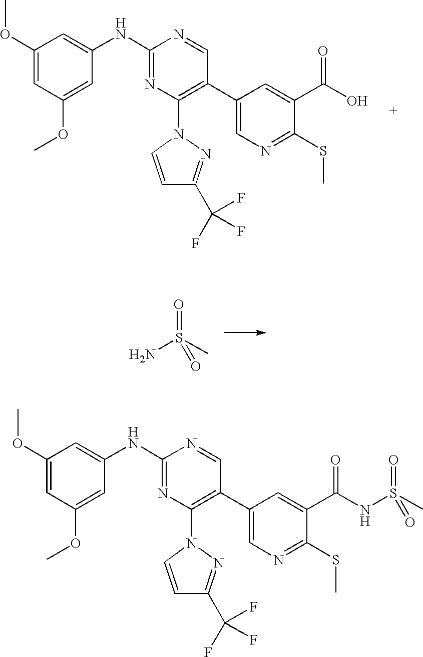

To a solution of 5-{2-[(3,5-dimethoxyphenyl)amino]-4-[3-(trifluoromethyl)-1H-pyrazol-1-yl]pyrimidin-5-yl}-2-(methylsulfanyl)pyridine-3-carboxylic acid (Example 851, 0.187 mmol, 0.1 g) in DCM was added TEA (0.56 mmol, 0.057 g), 2-chloro-1-methylpyridinium iodide (0.225 mmol, 0.0575 g), DMAP (0.037 mmol, 5 mg) and methanesulfonamide (0.28 mmol, 0.0268 g) stirred at RT for 2 h. The reaction mixture was diluted with DCM (20 mL), washed with water, 10% citric acid and brine, dried over Na₂SO₄, filtered and concentrated. The crude material was stirred with MeOH and hexane to give the pure title compound (0.1 g).

| | | Mass spectrum and ¹H | |
|---|---|---|---|
| Compound | Structure | NMR | SM |
| Example 852 | 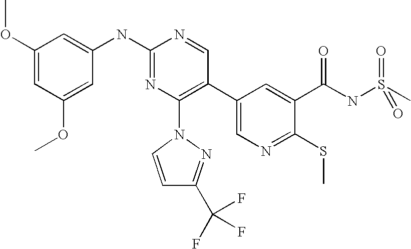 5-{2-[(3,5-dimethoxyphenyl)amino]-4-[3- (trifluoromethyl)-1H-pyrazol-1-yl]pyrimidin-5-yl}-2- (methylsulfanyl)-N-(methylsulfonyl)pyridine-3-carboxamide | MS(ES): 610 (M + 1) for C₂₄H₂₂F₃N₇O₅S₂. 400 MHz, DMSO-d₆: δ 2.41 (s, 3H), 3.27 (s, 3H), 3.74 (s, 6H), 6.20 (d, J = 1.92 Hz, 1H), 7.05 (d, J = 2.56 Hz, 1H), 7.11 (d, J = 2.04 Hz, 2H), 8.10 (d, J = 2.00 Hz, 1H), 8.25 (d, J = 2.04 Hz, 1H), 8.43 (s, 1H), 8.82 (s, 1H), 10.25 (s, 1H), 12.27 (s, 1H). | Example 851 5-{2-[(3,5-dimethoxy- phenyl)amino]-4-[3- (trifluoromethyl)-1H- pyrazol-1-yl]pyrimidin- 5-yl}-2- (methylsulfanyl)pyridine- 3-carboxylic acid |

### Example 853

### Methyl 5-{2-[(3,5-dimethoxyphenyl)amino]-4-[5-methyl-3-(trifluoromethyl)-1H-pyrazol-1-yl]pyrimidin-5-yl}-2-(methylsulfanyl)pyridine-3-carboxylate

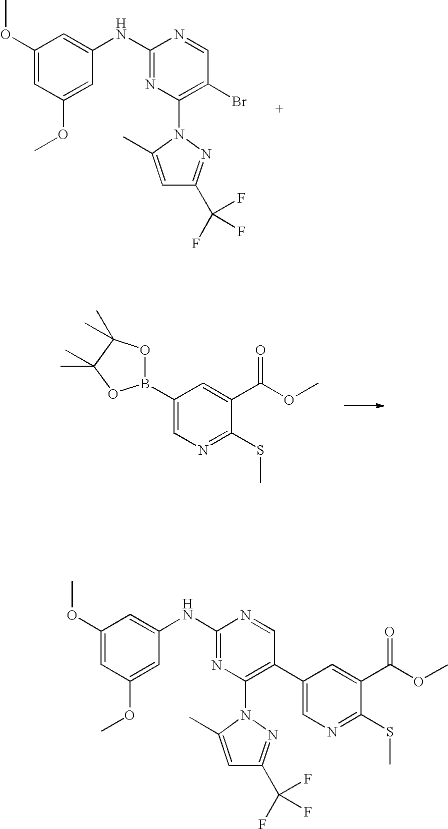

A suspension of 5-bromo-N-(3,5-dimethoxyphenyl)-4-[5-methyl-3-(trifluoromethyl)-1H-pyrazol-1-yl]pyrimidin-2-amine (Intermediate 216, 0.43 mmol, 0.2 g), a mixture of methyl 2-(methylsulfanyl)-5-(4,4,5,5-tetramethyl-1,3,2-dioxaborolan-2-yl)pyridine-3-carboxylate and [5-(methoxycarbonyl)-6-(methylsulfanyl)pyridin-3-yl]boronic acid (Intermediate 359, 0.56 mmol, 0.175 g), [1,1′-bis(diphenylphosphino)ferrocene]dichloropalladium(II) complex with dichloromethane (0.04 mmol, 0.036 g) and sodium carbonate (0.43 mmol, 0.0462 g) in acetonitrile/water (5:1) was degassed and heated to 90° C. for 30 min under an inert atmosphere. The reaction mass was passed through a diatomaceous earth bed and the solvent was concentrated in vacuo. The resultant residue taken in EtOAc (50 mL) was washed with water, brine, dried over Na₂SO₄, filtered and concentrated. The crude mass was further purified by silica gel column chromatography with (25% EtOAc/hexanes) to yield 0.180 g of the product.

| | | Mass spectrum and ¹H | |
|---|---|---|---|
| Compound | Structure | NMR | SM |
| Example 853 | 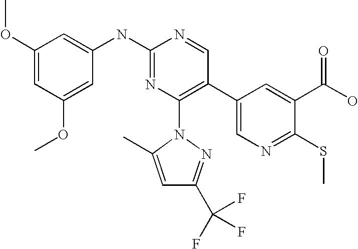 Methyl 5-{2-[(3,5-dimethoxyphenyl)-amino]-4-[5-methyl-3- (trifluoromethyl)-1H-pyrazol-1-yl]pyrimidin-5-yl}-2- (methylsulfanyl)pyridine-3-carboxylate | MS(ES): 561 (M + 1) for C₂₅H₂₃F₃N₆O₄S. 400 MHz, DMSO-d₆: δ 2.29 (s, 3H), 2.44 (s, 3H), 3.71 (s, 6H), 3.77 (s, 3H), 6.21 (s, 1H), 6.76 (s, 1H), 7.04 (s, 2H), 7.64 (d, J = 2.92 Hz, 1H), 8.53 (s, 1H), 8.98 (s, 1H), 10.24 (s, 1H). | Intermediate 216 5-Bromo-N-(3,5- dimethoxyphenyl)-4-[5- methyl-3- (trifluoromethyl)-1H- pyrazol-1-yl]pyrimidin- 2-amine |

### Example 854

### 5-{2-[(3,5-dimethoxyphenyl)amino]-4-[5-methyl-3-(trifluoromethyl)-1H-pyrazol-1-yl]pyrimidin-5-yl}-2-(methylsulfanyl)pyridine-3-carboxylic acid

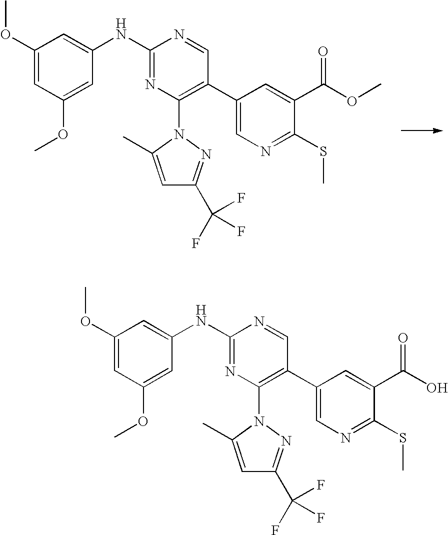

To 50 mg of methyl 5-{2-[(3,5-dimethoxyphenyl)amino]-4-[5-methyl-3-(trifluoromethyl)-1H-pyrazol-1-yl]pyrimidin-5-yl}-2-(methylsulfanyl)pyridine-3-carboxylate (Example 853, 0.089 mmol) taken in a mixture of THF (5 mL) and water (5 mL), was added sodium hydroxide (0.22 mmol, 9 mg) and stirred at room temperature for 3 h. The mixture was carefully acidified with 1 N HCl and the solid obtained was filtered and then dried to yield the product (0.04 g).

| | | Mass spectrum and ¹H | |
|---|---|---|---|
| Compound | Structure | NMR | SM |
| Example 854 | 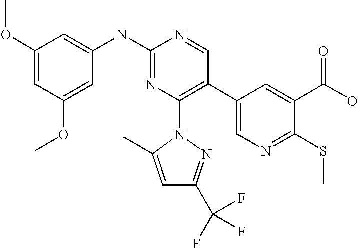 5-{2-[(3,5-Dimethoxyphenyl)amino]-4-[5-methyl-3- (trifluoromethyl)-1H-pyrazol-1-yl]pyrimidin-5-yl}-2- (methylsulfanyl)pyridine-3-carboxylic acid | MS(ES): 547 (M + 1) for C₂₄H₂₁F₃N₆O₄S. 400 MHz, DMSO-d₆: δ 2.34 (s, 3H), 2.42 (s, 3H), 3.73 (s, 6H), 6.23 (s, 1H), 6.77 (s, 1H), 7.06 (s, 2H), 7.68 (s, 1H), 8.49 (s, 1H), 8.98 (s, 1H), 10.24 (s, 1H), 13.37 (s, 1H). | Example 823 methyl 5-{2-[(3,5- dimethoxyphenyl)amino]- 4-[5-methyl-3- (trifluoromethyl)-1H- pyrazol-1-yl]pyrimidin- 5-yl}2-(methylsulfanyl)- pyridine-3-carboxylate |

### Example 855

### 5-{2-[(3,5-dimethoxyphenyl)amino]-4-[5-methyl-3-(trifluoromethyl)-1H-pyrazol-1-yl]pyrimidin-5-yl}-2-(methylsulfanyl)-N-(methylsulfonyl)pyridine-3-carboxamide

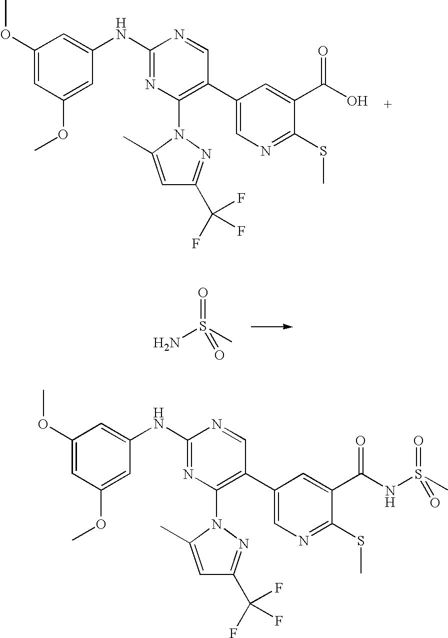

To a solution of 5-{2-[(3,5-dimethoxyphenyl)amino]-4-[5-methyl-3-(trifluoromethyl)-1H-pyrazol-1-yl]pyrimidin-5-yl}-2-(methylsulfanyl)pyridine-3-carboxylic acid (Example 854, 0.219 mmol, 0.12 g) in DCM was added TEA (0.65 mmol, 0.066 g), 2-chloro-1-methylpyridinium iodide (0.263 mmol, 0.0673 g) (0.225 mmol, 0.0575 g), DMAP (0.0439 mmol, 6 mg) and Methanesulfonamide (0.329 mmol, 0.0313 g), and stirred at RT for 2 h. The reaction mixture was diluted with DCM (20 mL), washed with water, 10% citric acid and brine, dried over Na₂SO₄, filtered and concentrated. The crude mass was further purified by silica gel column chromatography with (2% MeOH/EtOAc). The material obtained from chromatography was stirred with DCM and hexane to yield 0.1 g of the pure product (100 mg).

| | | Mass spectrum and ¹H | |
|---|---|---|---|
| Compound | Structure | NMR | SM |
| Example 855 | 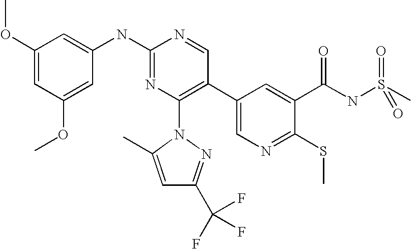 5-{2-[(3,5-dimethoxyphenyl)amino]-4-[5-methyl-3- (trifluoromethyl)-1H-pyrazol-1-yl]pyrimidin-5-yl}-2- (methylsulfanyl)-N-(methylsulfonyl)pyridine-3-carboxamide | MS(ES): 624 (M + 1) for C₂₅H₂₄F₃N₇O₅S₂. 400 MHz, DMSO-d₆: δ 2.34 (s, 3H), 2.40 (s, 3H), 3.35 (s, 3H), 3.72 (s, 6H), 6.21 (t, J = 2.08 Hz, 1H), 6.80 (s, 1H), 7.05 (d, J = 1.88 Hz, 2H), 7.97 (d, J = 2.00 Hz, 1H), 8.03 (d, J = 2.08 Hz, 1H), 9.01 (s, 1H),10.29 (s, 1H), 12.31 (s, 1H). | Example 854 5-{2-[(3,5- dimethoxyphenyl)amino]- 4-[5-methyl-3- (trifluoromethyl)-1H- pyrazol-1-yl]pyrimidin-5-yl}- 2-(methylsulfanyl)- pyridine-3- carboxylic acid |

### Example 856

### 2-methoxy-5-{2-[(3-methoxy-5-methylphenyl)amino]-4-[3-(trifluoromethyl)-1H-pyrazol-1-yl]pyrimidin-5-yl}-N-(methylsulfonyl)pyridine-3-carboxamide

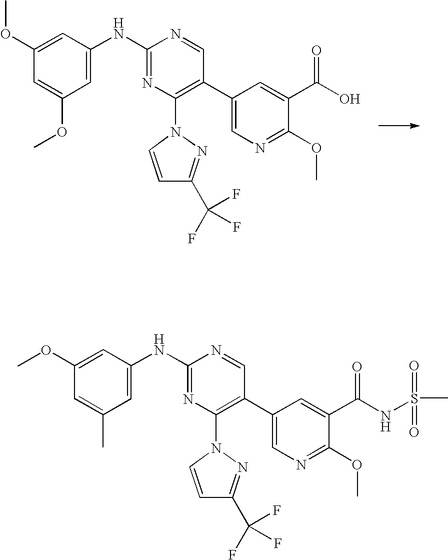

To a solution of 2-methoxy-5-{2-[(3-methoxy-5-methylphenyl)amino]-4-[3-(trifluoromethyl)-1H-pyrazol-1-yl]pyrimidin-5-yl}pyridine-3-carboxylic acid (Example 760, 0.33 mmol, 165 mg) in CH₂Cl₂ (10 mL), were added methanesulfonamide (0.82 mmol, 78 mg), triethylamine (0.99 mmol, 0.14 mL), 2-chloro-1-methylpyridinium iodide (0.41 mmol, 105 mg) and 4-(Dimethylamino)pyridine (0.066 mmol, 8 mg) and stirred at RT for 2 h. The reaction mixture was diluted with dichloromethane (20 mL) and further washed with 25% citric acid solution (2×15 mL), water (25 mL) and brine (25 mL). The organic layer was dried over Na₂SO₄ and concentrated. The crude mass was purified by 60-120 mesh silica (product eluted with 1-2% MeOH in CHCl₃) to afford 65 mg of the title compound as a white solid.

| | | Mass spectrum and ¹H | |
|---|---|---|---|
| Compound | Structure | NMR | SM |
| Example 856 | 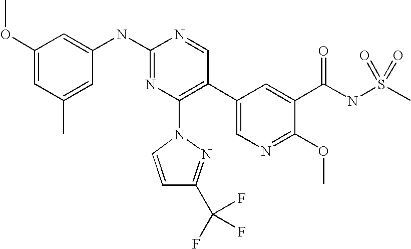 2-methoxy-5-{2-[(3-methoxy-5-methylphenyl)amino]- 4-[3-(trifluoromethyl)-1H-pyrazol-1-yl]pyrimidin-5-yl}-N- (methylsulfonyl)pyridine-3-carboxamide | MS(ES): 578 (M + 1) for C₂₄H₂₂F₃N₇O₅. 400 MHz, MeOD: δ 2.33 (s, 3H), 3.36 (s, 3H), 3.80 (s, 3H), 4.14 (s, 3H), 6.49 (s, 1H), 6.83 (d, J = 2.68 Hz, 1H), 7.07 (s, 1H), 7.33 (s, 1H), 8.09 (d, J = 2.48 Hz, 1H), 8.24 (d, J = 2.48 Hz, 1H), 8.58 (s, 2H). | Example 760 2-methoxy-5-{2-[(3-methoxy- 5-methylphenyl)-amino]- 4-[3-(trifluoromethyl)-1H- pyrazol-1-yl]pyrimidin- 5-yl}pyridine-3- carboxylic acid |

### Example 857

### 2-methoxy-5-{2-[(3-methoxy-5-methylphenyl)amino]-4-[5-methyl-3-(trifluoromethyl)-1H-pyrazol-1-yl]pyrimidin-5-yl}-N-(methylsulfonyl)pyridine-3-carboxamide

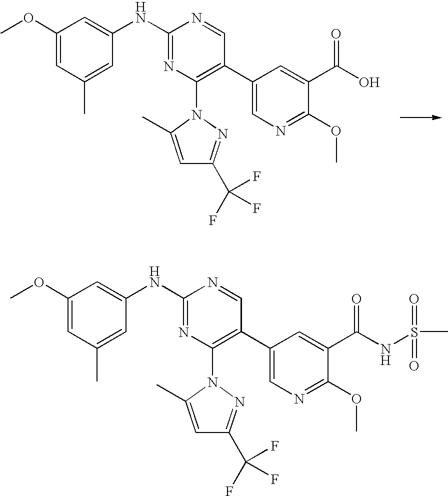

To a solution of 2-methoxy-5-{2-[(3-methoxy-5-methylphenyl)amino]-4-[5-methyl-3-(trifluoromethyl)-1H-pyrazol-1-yl]pyrimidin-5-yl}pyridine-3-carboxylic acid (Example 761, 0.36 mmol, 185 mg) in CH₂Cl₂ (10 mL), was added methanesulfonamide (0.90 mmol, 86 mg), triethylamine (1.08 mmol, 0.15 mL), 2-chloro-1-methylpyridinium iodide (0.45 mmol, 115 mg) and 4-(Dimethylamino)pyridine (0.072 mmol, 8.78 mg) and stirred at RT for 2 h. The reaction mixture was diluted with dichloromethane (20 mL) and further washed with 25% citric acid solution (2×15 mL), water (25 mL) and brine (25 mL). The organic layer was dried over Na₂SO₄ and concentrated. The crude mass was purified by 230-400 mesh silica (product eluted with 1-2% MeOH in CHCl₃) to afford 130 mg of white solid of the title compound.

| | | Mass spectrum and ¹H | |
|---|---|---|---|
| Compound | Structure | NMR | SM |
| Example 857 | 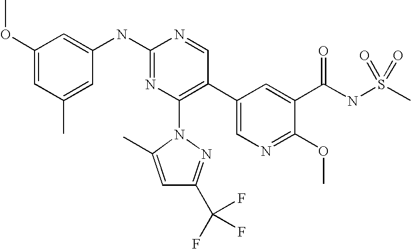 2-methoxy-5-{2-[(3-methoxy-5-methylphenyl)amino]- 4-[5-methyl-3-(trifluoromethyl)-1H-pyrazol-1- yl]pyrimidin-5-yl}-N-(methylsulfonyl)pyridine-3-carboxamide | MS (ES): 592 (M + 1) for C₂₅H₂₄F₃N₇O₅S. 400 MHz, CDCl₃: δ 2.36 (s, 3H), 2.48 (s, 3H), 3.40 (s, 3H), 3.82 (s, 3H), 4.18 (s, 3H), 6.44 (s, 1H), 6.52 (s, 1H), 6.93 (s, 1H), 7.19 (s, 1H), 7.35 (s, 1H), 8.07-8.09 (m, 2H), 8.63 (s, 1H), 10.10 (s, 1H). | Example 761 2-methoxy-5-{2-[(3-methoxy- 5-methylphenyl)amino]- 4-[5-methyl-3- (trifluoromethyl)-1H- pyrazol-1-yl]pyrimidin- 5-yl}pyridine-3- carboxylate acid |

### Example 858

### methyl 5-{2-[(3-methoxy-5-methylphenyl)amino]-4-[3-(trifluoromethyl)-1H-pyrazol-1-yl]pyrimidin-5-yl}-1-methyl-2-oxo-1,2-dihydropyridine-3-carboxylate

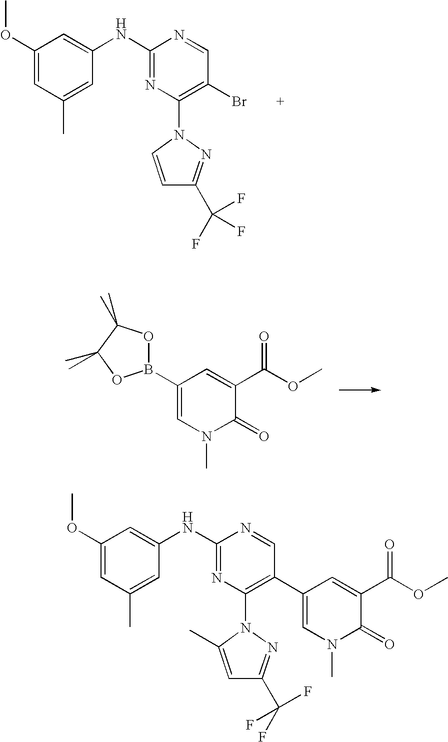

A solution of 5-bromo-N-(3-methoxy-5-methylphenyl)-4-[3-(trifluoromethyl)-1H-pyrazol-1-yl]pyrimidin-2-amine (Intermediate 293, 0.93 mmol, 400 mg), a mixture of methyl 1-methyl-2-oxo-5-(4,4,5,5-tetramethyl-1,3,2-dioxaborolan-2-yl)-1,2-dihydropyridine-3-carboxylate and [5-(methoxycarbonyl)-1-methyl-6-oxo-1,6-dihydropyridin-3-yl]boronic acid (Intermediate 323, 1.1 mmol based on the boronic ester, 328 mg), [1,1′-bis(diphenylphosphino)ferrocene]dichloropalladium(II)complex with CH₂Cl₂ (0.18 mmol, 146 mg) and sodium carbonate (0.9 mmol, 95 mg) in acetonitrile (40 mL)/water (10 mL) was degassed and heated to 90° C. for 30 minutes under nitrogen. The reaction mixture was concentrated in vacuo. The residue obtained was taken in ethyl acetate, washed with water and brine, dried over sodium sulfate and concentrated. The crude mass was purified by silica gel column chromatography (60-120 mesh) using 2% methanol/chloroform to obtain 300 mg of the title compound.

| | | Mass spectrum and ¹H | |
|---|---|---|---|
| Compound | Structure | NMR | SM |
| Example 858 | 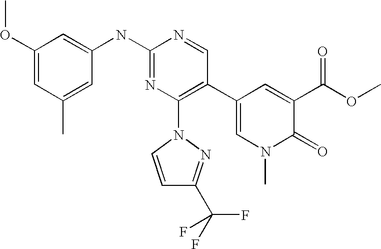 methyl 5-{2-[(3-methoxy-5-methylphenyl)amino]- 4-[3-(trifluoromethyl)-1H-pyrazol-1-yl]pyrimidin-5-yl}-1- methyl-2-oxo-1,2-dihydropyridine-3-carboxylate | MS(ES): 515 (M + 1) for C₂₄H₂₁F₃N₆O₄. 400 MHz, DMSO-d₆: δ 2.26 (s, 3H), 3.32 (s, 3H), 3.72 (s, 3H), 3.92 (s,3H), 6.44 (s, 1H), 7.06 (d, J = 3.24 Hz, 1H), 7.12 (s, 1H), 7.32 (s, 1H), 7.62 (d, J = 3.48 Hz, 1H), 8.16 (d, J = 4.24 Hz, 1H), 8.54 (s, 1H), 8.70 (s, 1H), 10.09 (s, 1H). | Intermediate 323 and Intermediate 293 5-bromo-N-(3-methoxy- 5-methylphenyl)-4-[3- (trifluoromethyl)-1H- pyrazol-1-yl]pyrimidin- 2-amine |

### Example 859

### 5-{2-[(3-methoxy-5-methylphenyl)amino]-4-[3-(trifluoromethyl)-1H-pyrazol-1-yl]pyrimidin-5-yl}-1-methyl-2-oxo-1,2-dihydropyridine-3-carboxylic acid

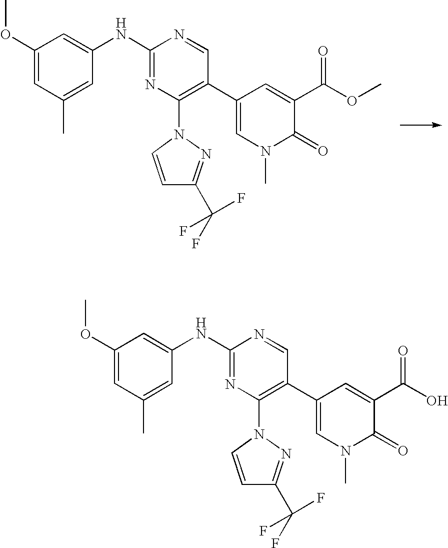

To 300 mg of methyl 5-{2-[(3-methoxy-5-methylphenyl)amino]-4-[3-(trifluoromethyl)-1H-pyrazol-1-yl]pyrimidin-5-yl}-1-methyl-2-oxo-1,2-dihydropyridine-3-carboxylate (Example 858, 0.58 mmol) taken in a mixture of THF (10 mL)/H₂O (10 mL), was added NaOH (1.1 mmol, 46 mg) and stirred at rt for 2 h. The solvent was removed in vacuo and the mixture was carefully acidified with 1 N HCl and the solid that precipitated was filtered and dried to obtain 190 mg of the title compound.

| | | Mass spectrum and ¹H | |
|---|---|---|---|
| Compound | Structure | NMR | SM |
| Example 859 | 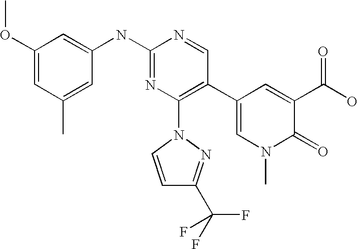 5-{2-[(3-methoxy-5-methylphenyl)amino]-4-[3- (trifluoromethyl)- 1H-pyrazol-1-yl]pyrimidin-5-yl}-1-methyl- 2-oxo-1,2-dihydropyridine-3-carboxylic acid | MS(ES): 501 (M + 1) for C₂₃H₁₉F₃N₆O₄. 400 MHz, DMSO-d₆: δ 2.29 (s, 3H), 3.70 (s, 3H), 3.75 (s, 3H), 6.47 (s,1H), 7.10 (s, 1H), 7.15 (s, 1H), 7.34 (s, 1H), 8.05 (d, J = 1.80 Hz, 1H), 8.44 (s, 1H), 8.60 (s, 1H), 8.72 (s, 1H), 10.17 (s, 1H), 14.53 (s, 1H). | Example 858 methyl 5-{2-[(3- methoxy-5-methyl- phenylamino]-4-[3- (trifluoromethyl)-1H- pyrazol-1-yl]pyrimidin- 5-yl}-1-methyl-2- oxo-1,2-dihydro- pyridine-3-carboxylate |

### Example 860

### methyl 5-{2-[(3-methoxy-5-methylphenyl)amino]-4-[5-methyl-3-(trifluoromethyl)-1H-pyrazol-1-yl]pyrimidin-5-yl}-1-methyl-2-oxo-1,2-dihydropyridine-3-carboxylate

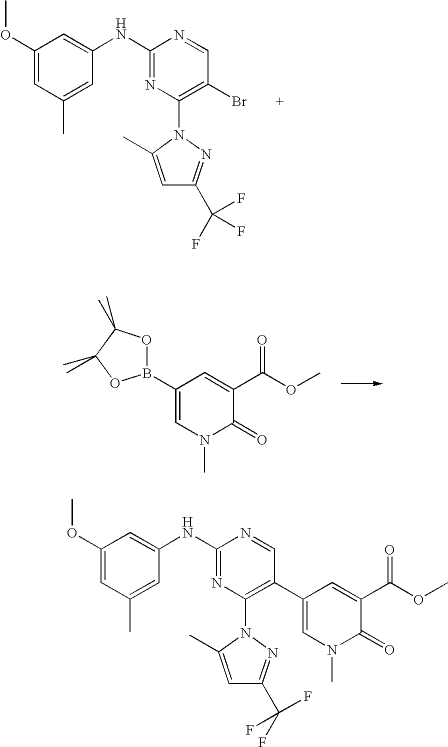

A solution 5-bromo-N-(3-methoxy-5-methylphenyl)-4-[5-methyl-3-(trifluoromethyl)-1H-pyrazol-1-yl]pyrimidin-2-amine (Intermediate 294, 0.79 mmol, 350 mg), a mixture of methyl 1-methyl-2-oxo-5-(4,4,5,5-tetramethyl-1,3,2-dioxaborolan-2-yl)-1,2-dihydropyridine-3-carboxylate (methoxycarbonyl)-1-methyl-6-oxo-1,6-dihydropyridin-3-yl]boronic acid (Intermediate 323, 0.95 mmol based on the boronic ester, 279 mg), [1,1′-bis(diphenylphosphino)ferrocene]dichloropalladium(II)complex with CH₂Cl₂ (0.14 mmol, 112 mg) and sodium carbonate (0.7 mmol, 74 mg) in acetonitrile (40 mL)/water (10 mL) was degassed and heated to 90° C. for 30 minutes under nitrogen. The reaction mixture was concentrated in vacuo. The residue obtained was taken in ethyl acetate, washed with water and brine, dried over sodium sulfate and concentrated. The crude mass was purified by silica gel column chromatography (60-120 mesh) using 2% methanol/chloroform to afford 300 mg of the title compound.

| | | Mass spectrum and ¹H | |
|---|---|---|---|
| Compound | Structure | NMR | SM |
| Example 860 | 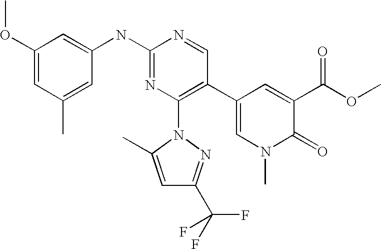 Methyl 5-{2-[(3-methoxy-5-methylphenyl)amino]- 4-[5-methyl-3-(trifluoromethyl)-1H-pyrazol-1- yl]pyrimidin-5-yl}-1-methyl-2-oxo-1,2-dihydropyridine-3- carboxylate | MS(ES): 529 (M + 1) for C₂₅H₂₃F₃N₆O₄. 300 MHz, CDCl₃: δ 2.35 (s, 3H), 2.38 (s, 3H), 3.56 (s, 3H), 3.81 (s, 3H), 3.88 (s, 3H), 6.45 (s, 1H), 6.51 (s, 1H), 6.90 (s, 1H), 7.18 (s, 1H), 7.52 (d, J = 2.76 Hz, 1H), 7.80 (d, J = 2.70 Hz, 1H), 8.58 (s, 1H). | Intermediate 323 and Intermediate 294 5-bromo-N-(3-methoxy-5- methylphenyl)-4-[5- methyl-3-(trifluoromethyl)- 1H-pyrazol-1-yl]pyrimidin- 2-amine |

### Example 861

### 5-{2-[(3-methoxy-5-methylphenyl)amino]-4-[5-methyl-3-(trifluoromethyl)-1H-pyrazol-1-yl]pyrimidin-5-yl}-1-methyl-2-oxo-1,2-dihydropyridine-3-carboxylic acid

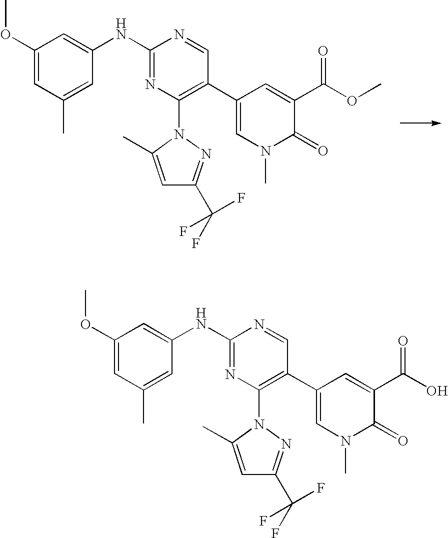

To 300 mg of methyl 5-{2-[(3-methoxy-5-methylphenyl)amino]-4-[5-methyl-3-(trifluoromethyl)-1H-pyrazol-1-yl]pyrimidin-5-yl}-1-methyl-2-oxo-1,2-dihydropyridine-3-carboxylate (Example 860, 0.57 mmol) dissolved in a mixture of THF (10 mL)/H₂O (10 mL), then NaOH (1.1 mmol, 46 mg) was added and the mixture was stirred at rt for 2 h. The solvent was removed in vacuo and the mixture was carefully acidified with 1 N HCl and the solid that precipitated was filtered and dried to obtain 200 mg of the title compound.

| | | Mass spectrum and ¹H | |
|---|---|---|---|
| Compound | Structure | NMR | SM |
| Example 861 | 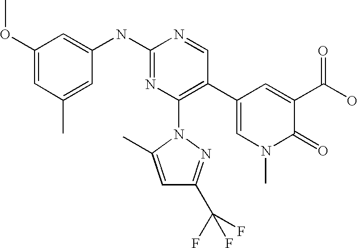 5-{2-[(3-methoxy-5- methylphenyl)amino]- 4-[5-methyl-3- (trifluoromethyl)-1H- pyrazol-1- yl]pyrimidin-5-yl}-1- methoxy-2-oxo-1,2- dihydropyridine-3- carboxylic acid | MS(ES): 515 (M + 1) for C₂₄H₂₁F₃N₆O₄, 400 MHz, DMSO-d₆: δ 2.27 (s, 3H), 2.50 (s, 3 H) 3.68 (s, 3H), 3.73 (s, 3 H) 6.48 (s, 1H), 6.82 (s, 1 H), 7.13 (s, 1H), 7.26 (s, 1 H) 7.64 (d, J = 2.40 Hz, 1H), 8.43 (d, J = 2.48 Hz, 1H), 8.86 (s, 1H), 10.16 (s, 1H), 14.41 (s, 1H). | Example 860 methyl 5-{2- [(3-methoxy- 5- methylphenyl)- amino]-4-[5- methyl-3- (trifluoro- methyl)-1H- pyrazol-1- yl]pyrimidin- 5-yl}-1- methyl-2- oxo-1,2- dihydro- pyridine-3- carboxylate |

### Example 862

### ethyl 1-ethyl-5-{2-[(3-methoxy-5-methylphenyl)amino]-4-[5-methyl-3-(trifluoromethyl)-1H-pyrazol-1-yl]pyrimidin-5-yl}-2-oxo-1,2-dihydropyridine-3-carboxylate

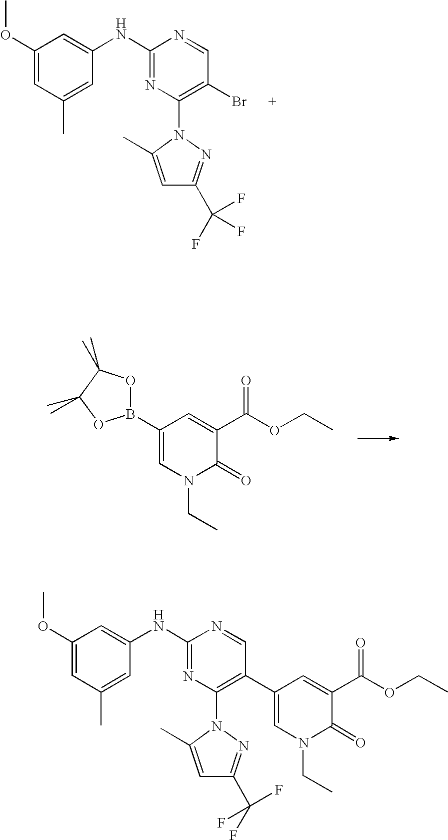

A solution 5-bromo-N-(3-methoxy-5-methylphenyl)-4-[5-methyl-3-(trifluoromethyl)-1H-pyrazol-1-yl]pyrimidin-2-amine (Intermediate 294, 0.9 mmol, 400 mg), a mixture of ethyl 1-ethyl-2-oxo-5-(4,4,5,5-tetramethyl-1,3,2-dioxaborolan-2-yl)-1,2-dihydropyridine-3-carboxylate and [5-(ethoxycarbonyl)-1-ethyl-6-oxo-1,6-dihydropyridin-3-yl]boronic acid (Intermediate 361, 1.1 mmol based on the boronic ester, 350 mg), [1,1′-bis(diphenylphosphino)ferrocene]dichloropalladium(II) (0.2 mmol, 146 mg) and sodium carbonate (0.9 mmol, 95 mg) in acetonitrile (40 mL)/water (10 mL) was degassed and heated to 90° C. for 30 minutes under nitrogen. The reaction mixture was concentrated in vacuo. The residue obtained was taken in ethyl acetate, washed with water and brine, dried over sodium sulfate and concentrated. The crude mass was purified by silica gel column chromatography (60-120 mesh) using 2% methanol/chloroform to yield 300 mg of the title compound.

| | | Mass spectrum and ¹H | |
|---|---|---|---|
| Compound | Structure | NMR | SM |
| Example 862 | 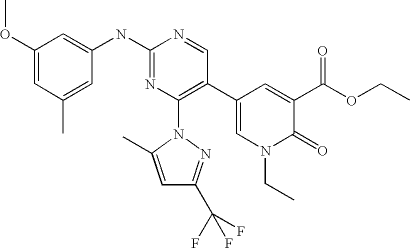 ethyl 1-ethyl-5-{2-[(3- methoxy-5- methylphenyl)amino]- 4-[5-methyl-3- (trifluoromethyl)-1H- pyrazol-1- yl]pyrimidin-5-yl}-2- oxo-1,2- dihydropyridine-3- carboxylate | MS(ES): 557 (M + 1) for C₂₇H₂₇F₃N₆O₄. 400 MHz, CDCl₃: δ 1.27- 1.37 (m, 6H), 2.34 (s, 6H), 3.80 (s, 3H), 3.97 (q, J = 7.16 Hz, 2H), 4.34 (q, J = 7.08 Hz, 2H), 6.44 (s, 1H), 6.50 (s, 1H), 6.89 (s, 1H), 7.19 (s, 1H), 7.39 (d, J = 2.48 Hz, 1H) 7.83 (d, J = 2.64 Hz, 1H), 8.59 (s, 1H). | Intermediate 361 and Intermediate 294 5-bromo-N- (3-methoxy- 5- methylphenyl)- 4-[5- methyl-3- (trifluoro- methyl)-1H- pyrazol-1- yl]pyrimidin- 2-amine |

### Example 863

### 1-ethyl-5-{2-[(3-methoxy-5-methylphenyl)amino]-4-[5-methyl-3-(trifluoromethyl)-1H-pyrazol-1-yl]pyrimidin-5-yl}-2-oxo-1,2-dihydropyridine-3-carboxylic acid

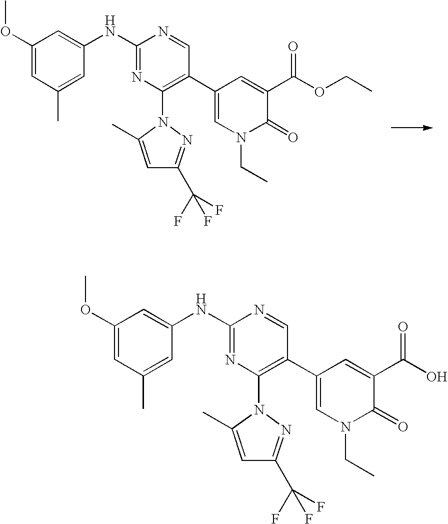

To 300 mg of ethyl 1-ethyl-5-{2-[(3-methoxy-5-methylphenyl)amino]-4-[5-methyl-3-(trifluoromethyl)-1H-pyrazol-1-yl]pyrimidin-5-yl}-2-oxo-1,2-dihydropyridine-3-carboxylate (Example 862, 0.54 mmol) taken in a mixture of THF (10 mL)/H₂O (10 mL), was added NaOH (1.1 mmol, 43 mg) and stirred at rt for 2 h. The solvent was removed in vacuo and the mixture was then carefully acidified with 1 N HCl and the solid that precipitate was filtered and dried to obtain 180 mg of the title compound.

| | | Mass spectrum and ¹H | |
|---|---|---|---|
| Compound | Structure | NMR | SM |
| Example 863 | 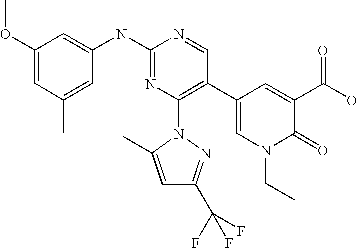 1-ethyl-5-{2-[(3- methoxy-5- methylphenyl)amino]- 4-[5-methyl-3- (trifluoromethyl)-1H- pyrazol-1- yl]pyrimidin-5-yl}-2- oxo-1,2- dihydropyridine-3- carboxylic acid | MS(ES): 529 (M + 1) for C₂₅H₂₃F₃N₆O₄. 400 MHz, DMSO-d₆: δ 1.27 (t, J = 7.08 Hz, 3H), 2.26 (s, 3H), 2.49 (s, 3H), 3.71 (s, 3H), 4.11 (t, J = 7.12 Hz, 2H), 6.46 (s, 1H), 6.79 (s, 1H), 7.12 (s, 1H), 7.24 (s, 1H), 7.78 (d, J = 2.44 Hz, 1H), 8.30 (d, J = 1.60 Hz, 1H), 8.88 (s, 1H), 10.15 (s, 1H), 14.45 (s, 1H). | Example 862 ethyl 1- ethyl-5-{2- [(3- methoxy-5- methylphenyl)- amino]- 4-[5- methyl-3- (trifluoro- methyl)-1H- pyrazol-1- yl]pyrimidin- 5-yl}-2- oxo-1,2- dihydropyridine- 3- carboxylate |

The compounds in the below table were prepared using the general method described above for Example 1 using Intermediate 113 and the specified starting material.

| Ex | Compound | Data | SM |
|---|---|---|---|
| Example 864 | 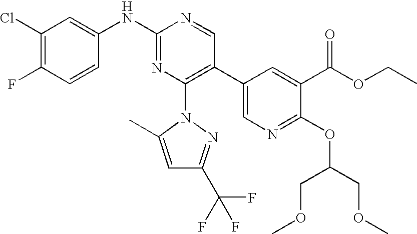 ethyl 5-(2-(3- chloro-4- fluorophenyl- amino)-4-(5- methyl-3- (trifluoromethyl)- 1H-pyrazol-1- yl)pyrimidin-5- yl)-2-(1,3- dimethoxypropan- 2- yloxy)nicotinate | MS(ES): 639.21 (M + H) for C28H27ClF4N6O5 1H NMR (400 MHz, DMSO-d6) δ ppm 1.25 (t, J = 7.06 Hz, 3 H) 3.26 (s, 6 H) 3.56 (d, J = 5.09 Hz, 4 H) 3.92 (s, 3 H) 4.21 (q, J = 7.16 Hz, 2 H) 5.49 (t, J = 4.99 Hz, 1 H) 6.78 (s, 1 H) 7.42 (t, J = 9.14 Hz, 1 H) 7.61 (d, J = 2.45 Hz, 1 H) 7.63- 7.75 (m, 1 H) 8.06 (d, J = 6.59 Hz, 1 H) 8.16 (d, J = 2.45 Hz, 1 H) 8.96 (s, 1 H) 10.43 (s, 1 H) | ethyl 2-(1,3- dimethoxy- propan-2-yloxy)- 5-(4,4,5,5- tetramethyl- 1,3,2- dioxaborolan- 2- yl)nicotinate Intermediate 315 |
| Example 865 | 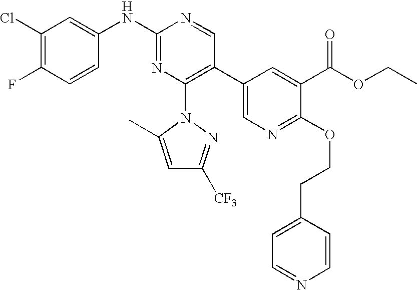 ethyl 5-(2-(3- chloro-4- fluorophenyl- amino)-4-(5- methyl-3- (trifluoromethyl)- 1H-pyrazol-1- yl)pyrimidin-5- yl)-2-(2- (pyridin-4- yl)ethoxy)- nicotinate | MS(ES): 640.39 (M − H) for C30H24ClF4N7O3 1H NMR (400 MHz, DMSO-d6) δ ppm 1.19 (t, J = 7.06 Hz, 3 H) 2.35 (s, 4 H) 3.06 (t, J = 6.12 Hz, 2 H)4.18 (q, J = 7.03 Hz, 2 H) 4.58 (t, J = 5.84 Hz, 2 H) 7.42 (t, J = 9.14 Hz, 1 H) 7.55 (d, J = 2.45 Hz, 1 H) 7.61-7.70 (m, 1 H) 8.06 (br. s., 1 H) 8.22 (d, J = 2.45 Hz, 1 H) 8.46 (d,J = 5.84 Hz, 2 H) 8.96 (s, 1 H) 10.44 (s, 1 H) | ethyl 2-(2- (pyridin-4- yl)ethoxy)-5- (4,4,5,5- tetramethyl- 1,3,2- dioxaborolan- 2-yl) nicotinate Intermediate 318 |

The compounds in the below table were prepared using the general method described above for Example 1 using Intermediate 115 and the specified starting material.

| Ex | Compound | Data | SM |
|---|---|---|---|
| Example 866 | 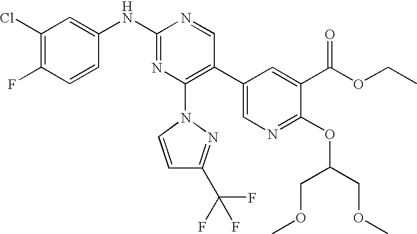 ethyl 5-(2-(3- chloro-4- fluorophenyl- amino)-4-(3- (trifluoromethyl)- 1H-pyrazol-1- yl)pyrimidin-5- yl)-2-(1,3- dimethoxypropan- 2- yloxy)nicotinate | MS(ES): 625.02 (M + H) for C27H25ClF4N6O5 1H NMR (400 MHz, DMSO-d6) δ ppm 1.25 (t, J = 7.06 Hz, 3 H) 3.30 (d, J = 9.42 Hz, 6 H) 3.59 (d, J = 5.09 Hz, 4 H) 4.19 (q, 2 H) 5.52 (t, J = 4.90 Hz, 1 H) 7.05 (d, J = 4.5 Hz, 1 H) 7.42 (t, J = 9.04 Hz, 1 H) 7.63-7.79 (m, 1 H) 7.84 (d, J = 2.45 Hz,1 H) 8.09 (d, J = 2.45 Hz, 1 H) 8.22 (d, J = 2.45 Hz, 1 H) 8.52 (s, 1 H) 8.81 (s, 1 H) 10.40 (s, 1 H) | ethyl 2-(1,3- dimethoxy- propan-2-yloxy)- 5-(4,4,5,5- tetramethyl- 1,3,2- dioxaborolan- 2- yl)nicotinate Intermediate 315 |
| Example 867 | 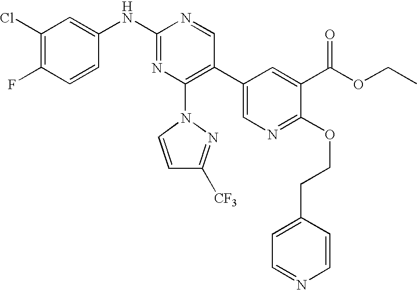 ethyl 5-(2-(3- chloro-4- fluorophenylamino)- 4-(3- (trifluoromethyl)- 1H-pyrazol-1- yl)pyrimidin-5- yl)-2-(2- (pyridin-4- yl)ethoxy)- nicotinate | MS(ES): 628.10 (M + H) for C29H22ClF4N7O3 1H NMR (400 MHz, DMSO-d6) δ ppm 1.12-1.28 (m, 6 H) 3.08 (br. s., 3 H) 3.93 (s, 3 H) 4.19 (q, J = 7.10 Hz, 3 H) 4.27 (d, J = 7.16 Hz, 1 H) 4.61 (s, 3 H) 7.05 (d, J = 2.45 Hz, 1 H) 7.32-7.49 (m, 5 H) 7.52 (br. s., 2 H) 7.69 (d, J = 2.64 Hz, 1 H) 7.80 (d, J = 2.26 Hz, 1H) 8.07 (d, J = 6.59 Hz, 1 H) 8.27 (d, J = 2.45 Hz, 1 H) 8.47 (br. s., 4 H) 8.80 (s, 1 H) 10.42 (s, 1 H) | ethyl 2-(2- (pyridin-4- yl)ethoxy)-5- (4,4,5,5- tetramethyl- 1,3,2- dioxaborolan- 2- yl)nicotinate Intermediate 318 |

The compound in the below table was prepared using the general method described above for Example 1 using Intermediate 216 and the specified starting material.

| Ex | Compound | Data | SM |
|---|---|---|---|
| Example 868 | 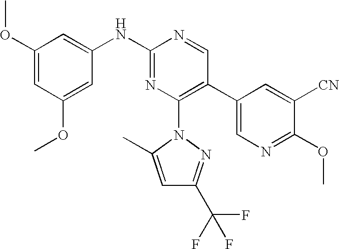 5-(2-(3,5- dimethoxy- phenylamino)-4-(5- methyl-3- (trifluoromethyl)- 1H-pyrazol-1- yl)pyrimidin-5- yl)-2- methoxy- nicotinitrile | MS(ES): 512.14 (M + H) for C24H20F3N7O3 1H NMR (400 MHz, DMSO-d6) δ ppm 2.46 (s, 3 H) 3.73 (s, 6 H) 3.99 (s, 3 H) 6.23 (s, 1 H) 6.81 (s, 1 H) 7.03 (d, J = 1.70 Hz, 2 H) 7.93 (d, J = 2.26 Hz, 1 H) 8.20 (d, J = 2.26 Hz, 1 H) 8.89 (s, 1 H) 10.21 (s, 1 H) | 2-methoxy-5- (4,4,5,5- tetramethyl- 1,3,2- dioxaborolan- 2- yl)nicotinonitrile Intermediate 362 |

The compounds in the below table were prepared using the general method described above for Example 214 using 1N sodium hydroxide (2 equivalents), dioxane:THF (1:1) as solvent and the specified starting material.

| Ex | Compound | Data | SM |
|---|---|---|---|
| Example 869 | 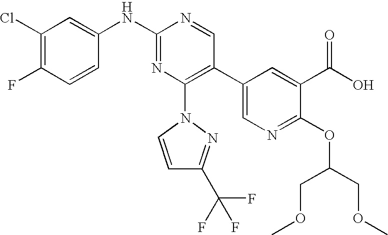 5-(2-(3-chloro- 4- fluorophenyl- amino)-4-(3- (trifluoromethyl)- 1H-pyrazol-1- yl)pyrimidin-5- yl)-2-(1,3- dimethoxypropan- 2- yloxy)nicotinic acid | MS(ES): 597.09 (M + H) for C25H21ClF4N6O5 1H NMR (400 MHz, DMSO-d6) δ ppm 3.28 (s, 6 H) 3.58 (br. s., 2 H) 3.60 (d, J = 1.70 Hz, 2 H) 5.52 (t, J = 4.99 Hz, 1 H) 7.04 (d, J = 2.45 Hz, 1 H) 7.42 (t, J = 9.04 Hz, 1 H) 7.70-7.80 (m, 1 H) 7.86 (s, 1 H) 8.07 (dd, J = 6.50, 2.54 Hz, 1 H) 8.17 (d, J = 2.26 Hz, 1 H) 8.47- 8.58 (m, 1H) 8.80 (s, 1 H) 10.40 (s, 1 H) 12.85 (br. s., 1 H) | ethyl 5-(2-(3- chloro-4- fluorophenyl amino)-4-(3- (trifluoro- methyl)-1H- pyrazol-1- yl)pyrimidin- 5-yl)-2-(1,3- dimethoxy- propan-2- yloxy)nicotinate Example 866 |
| Example 870 | 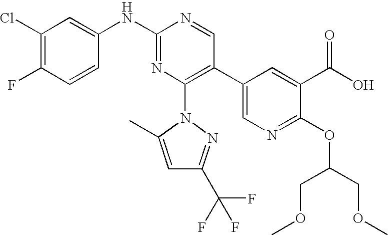 5-(2-(3-chloro- 4- fluorophenylamino)- 4-(5- methyl-3- (trifluoromethyl)- 1H-pyrazol-1- yl)pyrimidin-5- yl)-2-(1,3- dimethoxypropan- 2- yloxy)nicotinic acid | MS(ES): 611.14 (M + H) for C26H23ClF4N6O5 1H NMR (400 MHz, DMSO-d6) δ ppm 2.37 (s, 3 H) 3.26 (s, 6 H) 3.43- 3.60 (m, 4 H) 5.49 (t, J = 4.99 Hz, 1 H) 6.76 (s, 1 H) 7.42(t, J = 9.14 Hz, 1 H) 7.61-7.76 (m, 2 H) 8.05 (d, J = 2.26 Hz, 1 H) 8.07 (d, J = 2.45 Hz, 1 H) 8.95 (s, 1 H) 10.42 (s, 1 H) 12.88 (br. s., 1 H) | ethyl 5-(2-(3- chloro-4- fluorophenyl amino)-4-(5- methyl-3- (trifluoro- methyl)-1H- pyrazol-1- yl)pyrimidin- 5-yl)-2-(1,3- dimethoxy- propan-2- yloxy)nicotinate Example 864 |
| Example 871 | 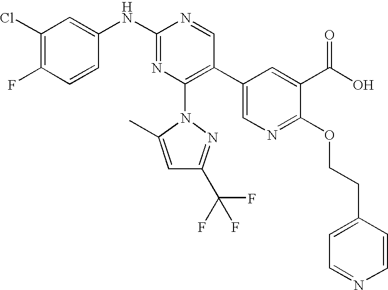 5-(2-(3-chloro- 4- fluorophenyl- amino)-4-(5- methyl-3- (trifluoromethyl) 1H-pyrazol-1- yl)pyrimidin-5- yl)-2-(2- (pyridin-4- yl)ethoxy)- nicotinic acid | MS(ES): 612.44 (M − H) for C28H20ClF4N7O3 1H NMR (400 MHz, DMSO-d6) δ ppm 2.34 (s, 3 H) 3.14 (t, J = 5.84 Hz, 2 H) 4.59 (t, J = 6.03 Hz, 2 H) 6.76 (s,1 H) 7.42 (t, J = 9.14 Hz, 1 H) 7.53-7.69 (m, 4 H) 8.06 (br. s., 1 H) 8.13 (s, 1H) 8.57 (d, J = 4.71 Hz, 2 H) 8.94 (s, 1 H) 10.44 (s, 1 H)12.96 (br. s., 1 H) | ethyl 5-(2-(3- chloro-4- fluorophenyl amino)-4-(5- methyl-3- (trifluoro- methyl)-1H- pyrazol-1- yl)pyrimidin- 5-yl)-2-(2- (pyridin-4- yl)ethoxy- nicotinate Example 865 |
| Example 872 | 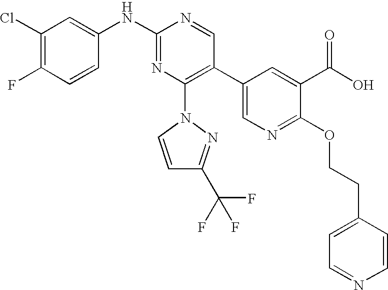 5-(2-(3-chloro- 4- fluorophenyl- amino)-4-(3- (trifluoromethyl)- 1H-pyrazol-1- yl)pyrimidin-5- yl)-2-(2- (pyridin-4- yl)ethoxy)- nicotinic acid | MS(ES): 600.04 (M + H) for C27H18ClF4N7O3 1H NMR (400 MHz, DMSO-d6) δ ppm 3.07 (m, 2 H) 4.59 (m, 2 H) 7.03 (m, 1 H) 7.38 (m, 3 H) 7.71 m, 1 H) 7.84 (d, J = 0.75 Hz, 1H) 8.08 (m., 1 H) 8.21 (m, 1 H) 8.47 (m, 3 H) 8.79 (m, 1 H) 10.35-10.51 (m, 1 H) 12.97 (m, 1 H) | ethyl 5-(2-(3- chloro-4- fluorophenyl amino)-4-(3- (trifluoromethyl)- 1H- pyrazol-1- yl)pyrimidin- 5-yl)-2-(2- (pyridin-4- yl)ethoxy)- nicotinate Example 867 |

### Example 873

### N-(3,5-dimethoxyphenyl)-5-(6-methoxy-5-(1H-tetrazol-5-yl)pyridin-3-yl)-4-(5-methyl-3-(trifluoromethyl)-1H-pyrazol-1-yl)pyrimidin-2-amine

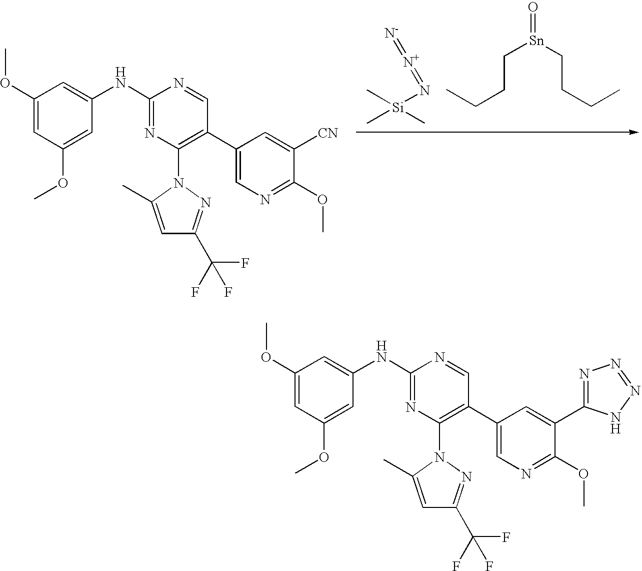

5-(2-(3,5-dimethoxyphenylamino)-4-(5-methyl-3-(trifluoromethyl)-1H-pyrazol-1-yl)pyrimidin-5-yl)-2-methoxynicotinonitrile (Example 868, 0.16 g, 0.31 mmol), dibutyltin oxide (0.023 g, 0.09 mmol), and TMS-N₃ (0.166 mL, 1.25 mmol) were suspended in 1,4-dioxane (1 mL) to give a yellow suspension. The mixture was heated in a microwave reactor at 140° C. for 1 h. Concentrated in vacuo. Purified by flash chromatography: 25 g silica gel column, 0-20% methanol in chloroform. Relevant fractions pooled and resulting material was dried under high vacuum to obtain the title compound as a tan foam in (0.1 g).

MS(ES): 555.17 (M+H) for C₂₄H₂₁F₃N₁₀O₃

¹H-NMR (400 MHz, DMSO-d6): δ ppm 2.39 (m, 3H) 3.73 (m., 6H) 4.04 (m, 3H) 6.22 (m, 1H) 6.75 (m., 1H) 7.06 (m, 2H) 8.03 (m, 1H) 8.18 (m., 1H) 8.96 (m, 1H) 10.22 (m, 1H).

### Example 874

### 5-(2-(3-chloro-4-fluorophenylamino)-4-(3-(trifluoromethyl)-1H-pyrazol-1-yl)pyrimidin-5-yl)-N-(methylsulfonyl)nicotinamide

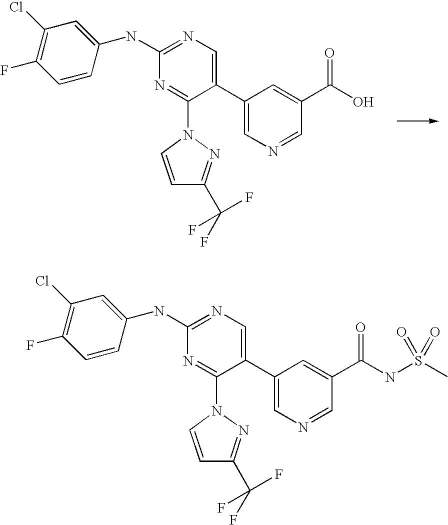

5-(2-(3-chloro-4-fluorophenylamino)-4-(3-(trifluoromethyl)-1H-pyrazol-1-yl)pyrimidin-5-yl)nicotinic acid (Example 320, 57 mg, 0.12 mmol) and 1,1-carbonyldiimidazole (50 mg) were combined in DMF (1.5 mL) and stirred for 30 minutes. Methanesulfonamide (16.99 mg, 0.18 mmol) was added and the mixture was stirred at 90° C. overnight. Purification by reverse phase chromatography (05-95% ACN/water NH4OH) gave the title compound (14 mg).

MS (Electrospray): 556.89, (MH⁺) for C₂₁H₁₄ClF₄N₇O₃S

¹H NMR (300 MHz, DMSO-d₆) δ: 2.84 (s, 3H) 7.00 (d, J=2.64 Hz, 1H) 7.42 (t, J=9.14 Hz, 1H) 7.74 (ddd, J=9.09, 4.19, 2.73 Hz, 1H) 8.00-8.13 (m, 2H) 8.28 (d, J=2.07 Hz, 1H) 8.45 (s, 1H) 8.78 (s, 1H) 8.95(d, J=1.70 Hz, 1H) 10.43 (s, 1H)

### Example 875

### (E)-3-(3-(2-(3-chloro-4-fluorophenylamino)-4-(3-cyclopropyl-1H-pyrazol-1-yl)pyrimidin-5-yl)phenyl)acrylic acid

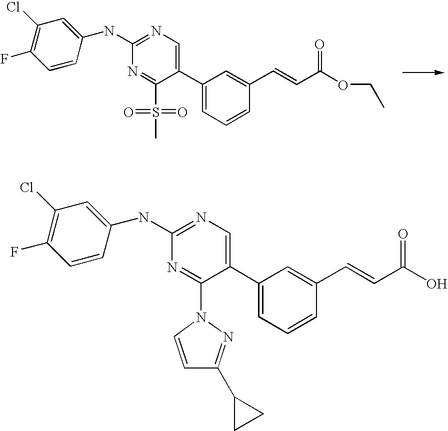

3-Cyclopropyl-1H-pyrazole (42.7 mg, 0.40 mmol) was dissolved in THF, then NaH (9.48 mg, 0.40 mmol) was added slowly and the mixture was stirred for 30 minutes. The mixture was added to a solution of (E)-ethyl 3-(3-(2-(3-chloro-4-fluorophenylamino)-4-(methylsulfonyl)pyrimidin-5-yl)phenyl)acrylate Intermediate 125 (94 mg, 0.20 mmol) in THF (2 mL). The resulting mixture was allowed to stir overnight at room temperature. Methanol (0.25 ml) then water (0.25 ml) were added and the mixture was evaporated. Purification using reverse phase chromatography (C18, 20 to 95% CH₃CN/H₂O/0.1% Trifluoroacetic acid) yielded the title compound. (17 mg).

MS (Electrospray): 476.90(MH⁺) for C₂₅H₁₉ClFN₅O₂

¹H NMR (300 MHz, DMSO-d₆) δ: 0.18-0.47 (m, 2H) 0.57-0.76 (m, 2H) 1.52-1.80 (m, 1H) 6.35 (d,J=2.64 Hz, 1H) 6.46 (s, 1H) 6.52 (s, 1H) 7.19 (d, J=7.54 Hz, 1H) 7.32-7.46 (m, 2H) 7.48-7.59 (m, 1H)7.62 (d, J=8.10 Hz, 1H) 7.72 (ddd, J=9.09, 4.19, 2.73 Hz, 1H) 8.06-8.21 (m, 2H) 8.58 (s, 1H) 10.16 (s, 1H)

### Example 876

### N-(3-chloro-4-fluorophenyl)-5-(6-methoxypyridin-2-yl)-4-(3-(trifluoromethyl)-1H-pyrazol-1-yl)pyrimidin-2-amine

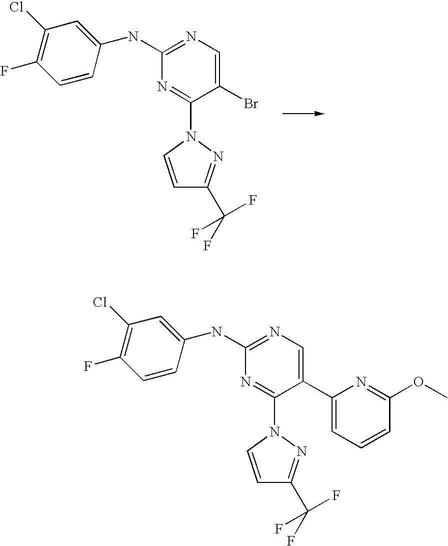

5-bromo-N-(3-chloro-4-fluorophenyl)-4-(3-(trifluoromethyl)-1H-pyrazol-1-yl)pyrimidin-2-amine Intermediate 115 (100 mg, 0.23 mmol), Pd(Ph3P)4 (79 mg, 0.07 mmol), and 0.5M (6-methoxypyridin-2-yl)zinc(II) bromide in THF (0.916 mL, 0.46 mmol) were combined in THF (6 mL) under argon. The reaction was heated at 65° C. for 45 minutes. Additional 0.5M (6-methoxypyridin-2-yl)zinc(II) bromide in THF (0.916 mL, 0.46 mmol) was added and the mixture was allowed to stir for an additional 1.5 hours. The reaction mixture was concentrated and purified by silica gel flash chromatography using 0-50% ethyl acetate in hexanes. The title compound was obtained as a solid. (7 mg)

MS (Electrospray): 467.80(MH⁺) C₂₀H₁₃ClF₄N₆O

1H NMR (300 MHz, DMSO-d6) [symbol] ppm 3.55 (s, 3H) 6.59-6.83 (m, 1H) 6.93-7.14 (m, 2H) 7.27-7.52 (m, 2H) 7.63-7.82 (m, 1H) 8.07-8.21 (m, 1H) 8.37-8.52 (m, 1H) 8.99 (s, 1H) 10.32-10.54 (m, 1H)

### Example 877

### 6-(2-(3-chloro-4-fluorophenylamino)-4-(3-(trifluoromethyl)-1H-pyrazol-1-yl)pyrimidin-5-yl)picolinic acid

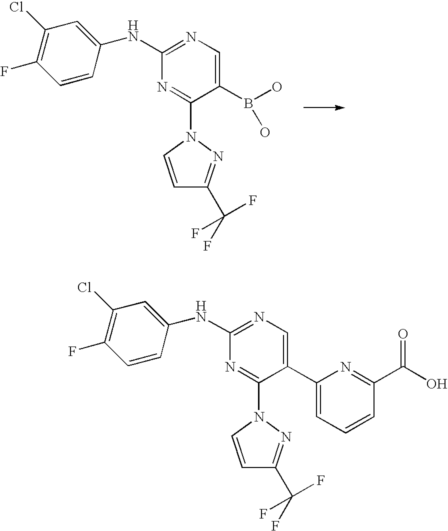

2-(3-chloro-4-fluorophenylamino)-4-(3-(trifluoromethyl)-1H-pyrazol-1-yl)pyrimidin-5-ylboronic acid Intermediate 364 (80 mg, 0.20 mmol), methyl 6-bromopicolinate (34.4 mg, 0.16 mmol), Pd(Ph₃P)₄ (46.0 mg, 0.04 mmol), and K₂CO₃ (41.3 mg, 0.30 mmol) were combined with Dioxane (4 mL) and water (1 mL). The mixture was heated in a microwave for 45 min. at 120° C. Reverse phase chromatography (C18, 20-95% CH₃CN/H₂O/0.1% Trifluoroacetic acid) of the crude mixture gave the title compound. (19 mg)

MS (Electrospray): 479.79 (MH⁺) C20H11ClF4N6O2

¹H NMR (300 MHz, DMSO-d6) δ ppm 7.04 (d, J=2.64 Hz, 1H) 7.42 (d, J=7.91 Hz, 2H) 7.70-7.83 (m, 1H) 7.88-8.03 (m, 2H) 8.06-8.15 (m, 1H) 8.97 (s, 1H) 10.50 (s, 1H) 12.67-13.30 (m, 1H)

### Example 878

### 3-(2-(3-chloro-4-fluorophenylamino)-4-(3-(trifluoromethyl)-1H-pyrazol-1-yl)pyrimidin-5-yl)-N-methyl-N-(methylsulfonyl)benzamide

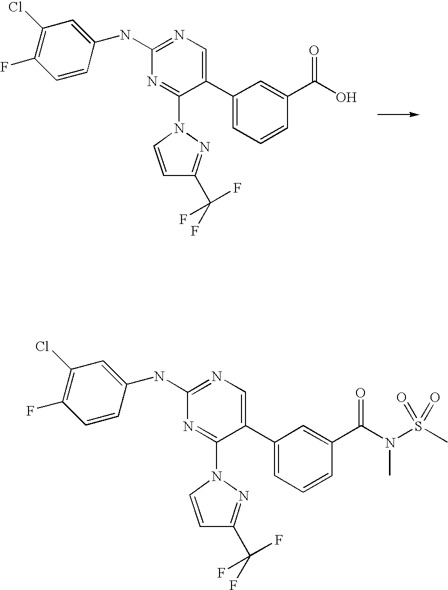

3-(2-(3-chloro-4-fluorophenylamino)-4-(3-(trifluoromethyl)-1H-pyrazol-1-yl)pyrimidin-5-yl)benzoic acid Example (45 mg, 0,09 mmol) was dissolved in DMF (3 ml). Triethylamine (0.033 ml, 0.24 mmol) and HATU (35 mg, 0.09 mmol) was added and the mixture was allowed to stir for 10 min. N-methylmethanesulfonamide (10 mg, 0.09 mmol) was added to the reaction mixture and it was allowed to stir at RT overnight. Purification using reverse phase chromatography (C18, 5-95% CH₃CN/H₂O/0.1% Trifluoroacetic acid) yielded the title compound. (12 mg).

MS (Electrospray): 569.93 (MH⁺) C₂₃H₁₇ClF₄N₆O₃S

¹H NMR (300 MHz, DMSO-d6) δ ppm 3.25-3.35 (s, 3H), 3.55 (s, 3H) 6.59-6.77 (m, 1H) 6.92-7.11 (m, 2H) 7.22-7.55 (m, 2H) 7.65-7.85 (m, 2H) 8.01-8.22 (m, 1H) 8.34-8.53 (m, 1H) 8.99 (s, 1H) 10.40-10.54 (m, 1H)

### Example 879

### 2′-(3-chloro-4-fluorophenylamino)-6-methyl-4′-(3-(trifluoromethyl)-1H-pyrazol-1-yl)-2,5′-bipyrimidine-4-carboxylic acid

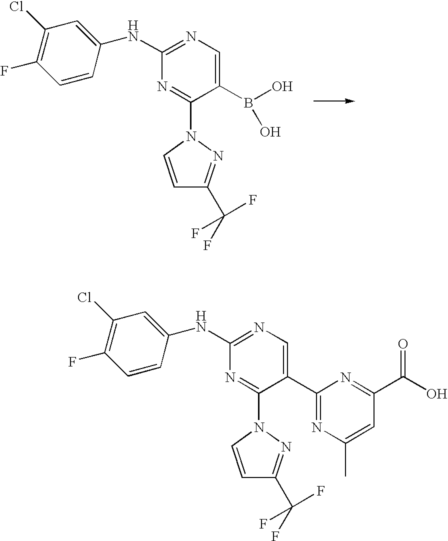

2-(3-chloro-4-fluorophenylamino)-4-(3-(trifluoromethyl)-1H-pyrazol-1-yl)pyrimidin-5-ylboronic acid Intermediate 364 (100 mg, 0.25 mmol), methyl 2-chloro-6-methylpyrimidine-4-carboxylate (93 mg, 0.50 mmol), Pd(Ph₃P)₄ (57.6 mg, 0.05 mmol), and K₂CO₃ (51.6 mg, 0.37 mmol) were combined with Dioxane (2 mL) and water (0.500 mL). The mixture was heated in a microwave for 45 min at 120° C. The mixture was purified using reverse phase chromatography (C18, 5-95%. CH₃CN/H₂O/0.1% Trifluoroacetic acid) to yield the title compound. (28 mg).

MS (Electrospray): 494.80 (MH⁺) C20H12C1F4N7O2

¹H NMR (300 MHz, DMSO-d6) δ ppm 2.46 (s, 3H) 6.95-7.10 (m, 1H) 7.38-7.49 (m, 1H) 7.73-7.80 (m,1H) 7.81 (s, 1H) 8.05-8.13 (m, 1H) 8.52-8.65 (m, 1H) 9.10 (s, 1H) 10.54-10.66 (m, 1H) 13.49-13.84 (m, 1H)

The compounds in the following table were prepared using the procedure for Example 1 with the specified starting materials.

| Example | Compound | Mass and NMR data | SM |
|---|---|---|---|
| Example 880 | 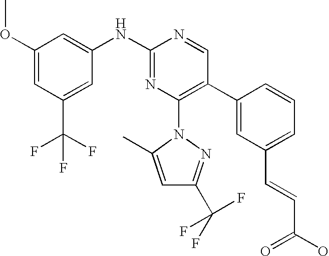 (E)-3-(3-(2-(3-methoxy-5- (trifluoromethyl)phenylamino)- 4-(5-methyl-3-(trifluoromethyl)- 1H-pyrazol-1-yl)pyrimidin-5- yl)phenyl)acrylic acid | MS: ES+ 564 for C₂₆H₁₉F₆N₅O₃ 1H NMR (300 MHz, DMSO-d₆) d ppm 2.21 (s, 3 H) 3.82 (s, 3 H) 6.45 (d, J = 16.01 Hz, 1 H) 6.72 (s, 1 H) 6.90 (s, 1 H) 7.06 (d, J = 7.91 Hz, 1 H) 7.31- 7.43 (m, 2 H) 7.50 (d, J = 16.01 Hz, 1 H) 7.61 (d, J = 7.72 Hz, 1 H) 7.71 (sl, 1 H) 7.82 (s, 1 H) 9.02 (s, 1 H) 10.56 (s, 1 H) 12.44 (br. s., 1 H) | Intermediate 375 and [3-(trans-2- carboxy vinyl)phenyl]boronic acid |
| Example 881 | 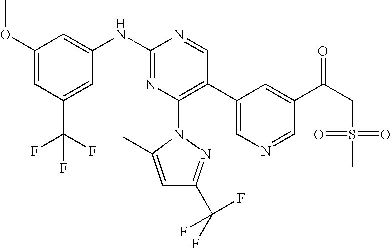 1-(5-(2-(3-methoxy-5- (trifluoromethyl)phenylamino)- 4-(5-methyl-3-(trifluoromethyl)- 1H-pyrazol-1-yl)pyrimidin-5- yl)pyridin-3-yl)-2- (methylsulfonyl)ethanone | MS: ES+ 615 for C₂₅H₂₀F₆N₆O₄S 1H NMR (300 MHz, DMSO-d₆) d ppm 2.45 (s, 3 H) 3.15 (s, 3 H) 3.83 (s, 3 H) 5.18 (br. s., 2 H) 6.79 (s, 1 H) 6.92 (s, 1 H) 7.68 (s, 1 H) 7.84 (s, 1 H) 8.21 (t, J = 1.98 Hz, 1 H) 8.40 (d, J = 2.07 Hz, 1 H) 9.06 (s, 1 H) 9.11 (d, J = 1.88 Hz, 1 H) 10.60 (s, 1 H) | Intermediate 375 and Intermediate 415 |
| Example 882 | 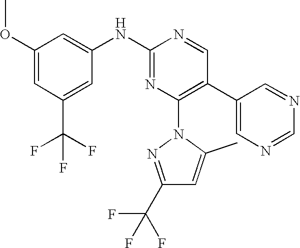 N-(3-methoxy-5- (trifluoromethyl)phenyl)-4-(5- methyl-3-(trifluoromethyl)-1H- pyrazol-1-yl)-5,5′-bipyrimidin-2- amine | MS: ES+ 496 for C₂₁H₁₅F₆N₇O 1H NMR (300 Mhz, DMSO-d₆) d ppm 2.53 (s, 3 H) 3.83 (s, 3 H) 6.82 (s, 1 H) 6.93 (s, 1 H) 7.66 (s, 1 H) 7.82 (s, 1 H) 8.55 (s, 2 H) 9.01 (s, 1H) 9.10 (s, 1 H) 10.57 (s, 1 H) | Intermediate 375 and pyrimidin-5-yl boronic acid |
| Example 883 | 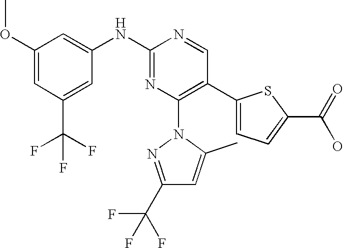 5-(2-(3-methoxy-5- (trifluoromethyl)phenylamino)- 4-(5-methyl-3-(trifluoromethyl)- 1H-pyrazol-1-yl)pyrimidin-5- yl)thiophene-2-carboxylic acid | MS: ES+ 544 for C₂₂H₁₅F₆N₅O₃S ¹H NMR (300 MHz, DMSO-d₆) d ppm 2.18 (s, 3 H) 3.81 (s, 3 H) 6.82 (s, 1 H) 6.89 (s, 1 H) 6.97 (d, J = 3.77 Hz, 1 H) 7.28 (d, J = 3.58 Hz, 1 H) 7.66-7.80 (m, 2 H) 9.14 (s, 1 H) 10.61 (br. s., 1 H) | Intermediate 375 and 5- boronothiophene-2- carboxylic acid |
| Example 884 | 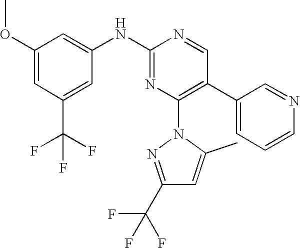 N-(3-methoxy-5- (trifluoromethyl)phenyl)-4-(5- methyl-3-(trifluoromethyl)-1H- pyrazol-1-yl)-5-(pyridin-3- yl)pyrimidin-2-amine | MS: ES+ 495 for C₂₂H₁₆F₆N₆O 1H NMR (300 MHz, DMSO-d₆) d ppm 2.46 (s, 3 H) 3.83 (s, 3 H) 6.79 (s, 1 H) 6.93 (s, 1 H) 7.61 (dd, J = 7.91, 5.09 Hz, 1 H) 7.67 (s, 1 H) 7.75-7.86 (m, 2 H) 8.49 (d, J = 1.70 Hz, 1 H) 8.64 (dd, J = 5.09, 1.13 Hz, 1 H) 8.99 (s, 1 H) 10.58 (s, 1 H) | Intermediate 375 and pyridin-3- ylboronic acid |
| Example 885 | 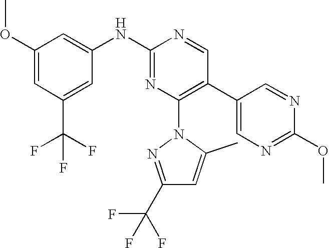 2′-methoxy-N-(3-methoxy-5- (trifluoromethyl)phenyl)-4-(5- methyl-3-(trifluoromethyl)-1H- pyrazol-1-yl)-5,5′-bipyrimidin-2- amine | MS: ES+ 526 for C₂₂H₁₇F₆N₇O₂ 1H NMR (300 Mhz, DMSO-d₆) d ppm 2.46 (s, 3 H) 3.83 (s, 3 H) 3.91 (s, 3 H) 6.80 (s, 1 H) 6.91 (s, 1 H) 7.66 (s, 1 H) 7.82 (s, 1 H) 8.33 (s, 2 H) 8.97 (s, 1 H) 10.53 (s, 1 H) | Intermediate 375 and 2- methoxypyrimidin- 5-ylboronic acid |
| Example 886 | 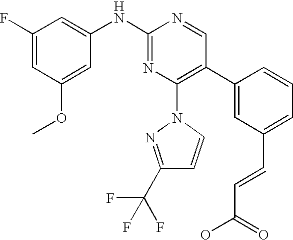 (E)-3-(3-(2-(3-fluoro-5- methoxyphenylamino)-4-(3- (trifluoromethyl)-1H-pyrazol-1- yl)pyrimidin-5-yl)phenyl)aceylic acid | MS: ES+ 500 for C₂₄H₁₇F₄N₅O₃ 1H NMR (300 MHz, DMSO-d₆) d ppm 3.77 (s, 3 H) 6.44- 6.48 (m, 1 H) 6.50 (s, 1 H) 6.99 (d, J = 2.64 Hz, 1 H) 7.15 (d, J = 7.72 Hz, 1 H) 7.29- 7.43 (m, 3 H) 7.49- 7.59 (m, 2 H) 7.64 (d, J = 7.91 Hz, 1 H) 8.33 (d, J = 1.70 Hz, 1 H) 8.84 (s, 1 H) 10.39 (s, 1 H) 12.38 (br. s., 1 H) | Intermediate 376 and 3-(2- Carboxyvinyl- benzeneboronic acid |
| Example 887 | 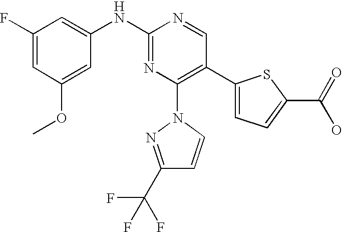 5-(2-(3-fluoro-5- methoxyphenylamino)-4-(3- (trifluoromethyl)-1H-pyrazol-1- yl)pyrimidin-5-yl)thiophene-2- carboxylic acid | MS: ES+ 480 for C₂₀H₁₃F₄N₅O₃S 1H NMR (300 MHz, DMSO-d₆) d ppm 3.77 (s, 3 H) 6.51 (dt, J = 10.83, 2.21 Hz, 1 H) 7.07 (d, J = 2.64 Hz, 1 H) 7.11 (d, J = 3.77 Hz, 1 H) 7.26- 7.34 (m, 2 H) 7.64 (d, J = 3.77 Hz, 1 H) 8.43 (d, J = 1.51 Hz, 1 H) 8.95 (s, 1 H) 10.52 (s, 1 H) 13.13 (br. s., 1 H) | Intermediate 376 and 5- boronothiophene-2- carboxylic acid |
| Example 888 | 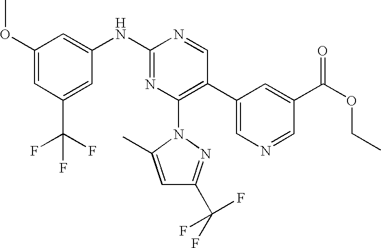 ethyl 5-(2-(3-methoxy-5- (trifluoromethyl)phenylamino)- 4-(5-methyl-3-(trifluoromethyl)- 1H-pyrazol-1-yl)pyrimidin-5- yl)nicotinate | MS: ES+ 567 for C₂₅H₂₀F₆N₆O₃ 1H NMR (300 MHz, DMSO-d₆) d ppm 1.30 (t, 3 H) 2.42 (s, 3 H) 3.83 (s, 3 H) 4.32 (q, J = 7.10 Hz, 2 H) 6.79 (s, 1 H) 6.92 (s, 1 H) 7.69 (s, 1 H) 7.79-7.85 (m, 2 H) 8.63 (d, J = 2.26 Hz, 1 H) 8.99 (d, J = 2.07 Hz, 1 H) 9.05 (s, 1 H) 10.59 (s, 1 H) | Intermediate 375 and ethyl 5-(4,4,5,5- tetramethyl-1,3,2- dioxaborolan-2- yl)nicotinate |
| Example 889 | 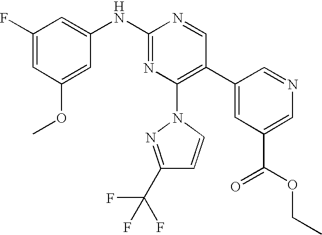 ethyl 5-(2-(3-fluoro-5- methoxyphenylamino)-4-(3- (trifluoromethyl)-1H-pyrazol-1- yl)pyrimidin-5-yl)nicotinate | MS: ES+ 503 for C₂₃H₁₈F₄N₆O₃ 1H NMR (300 MHz, DMSO-d₆) d ppm 1.31 (t, J = 7.16 Hz, 3 H) 3.78 (s, 3 H) 4.34 (q, J = 7.16 Hz, 2 H) 6.51 (dt, J = 10.93, 2.26 Hz, 1 H) 7.05 (d, J = 2.64 Hz, 1 H) 7.27- 7./37 (m, 2 H) 8.04 (t, J = 2.17 Hz, 1 H) 8.54 (d, J = 1.88 Hz, 1 H) 8.69 (d, J = 2.26 Hz, 1 H) 8.86 (s, 1 H) 9.03 (d, J = 2.07 Hz, 1 H) 10.43 (s, 1 H) | Intermediate 376 and 3-(ethoxy- carbonyl) pyridine-5-boronic acid pinacol ester |
| Example 890 | 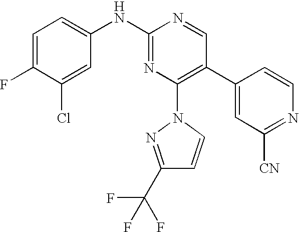 4-(2-(3-chloro-4- fluorophenylamino)-4-(3- (trifluoromethyl)-1H-pyrazol-1- yl)pyrimidin-5-yl)picolinonitrile | MS: ES+ 460 for C₂₀H₁₀ClF₄N₇ 1H NMR (300 MHz, DMSO-d₆) δ ppm 7.10 (d, J = 2.64 Hz, 1 H) 7.444 (t, J = 9.14 Hz, 1 H) 7.60 (dd, J = 5.18, 1.79 Hz, 1 H) 7.74 (ddd, J = 9.04, 4.24, 2.73 Hz, 1 H) 7.90 (d, J = 0.94 Hz, 1 H) 8.05 (dd, J = 6.78, 2.64 Hz, 1 H) 8.59 (dd, J = 2.54, 0.85 Hz, 1 H) 8.72 (d, J = 5.09 Hz, 1 H) 8.83 (s, 1 H) 10.54 (s, 1 H) | Intermediate 115 and 4-(4,4,5,5- tetramethyl-1,3,2- dioxaborolan-2-yl) Picolinonitrile |
| Example 891 | 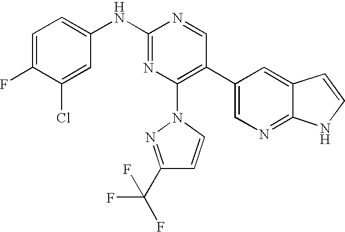 N-(2-chloro-4-fluorophenyl)-5- (1H-pyrrolo[2,3-b]pyridin-5-yl)- 4-(3-(trifluoromethyl)-1H- pyrazol-1-yl)pyrimidin-2-amine | MS: ES+ 474 for C₂₁H₁₂ClF₄N₇ 1H NMR (300 MHz, DMSO-d₆) d ppm 6.44 (dd, 1 H) 6.95 (d, J = 2.45 Hz, 1 H) 7.41 (t,J = 9.14 Hz, 1 H) 7.49 (t, 1 H) 7.72 (ddd, J = 9.115, 4.05, 2.83 Hz, 1 H) 7.82 (d, J = 1.88 Hz, 1 H) 7.93 (d, J = 1.88 Hz, 1 H) 8.14 (dd, J = 6.78, 2.64 Hz, 1 H) 8.28 (s, 1 H) 8.82 (s, 1 H) 10.38 (s, 1 H) 11.71 (br. s., 1 H) | Intermediate 115 and 5-(4,4,5,5-tetra methyl-1,3,2- dioxaborolan-2-yl)- 1H-pyrrolo[2,3-b] pyridine |
| Example 892 | 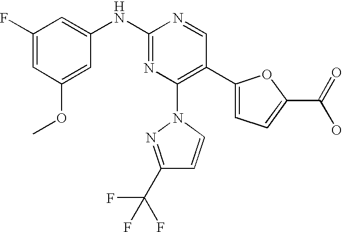 5-(2-(3-fluoro-5- methoxyphenylamino)-4-(3- (trifluoromethyl)-1H-pyrazol-1- yl)pyrimidin-5-yl)furan-2- carboxylic acid | MS: ES+ 464 for C₂₀H₁₃F₄N₅O₄ 1H NMR (300 MHz, DMSO-d₆) d ppm 3.77 (s, 3 H) 6.50 (dt, J = 10.93, 2.26 Hz, 1 H) 6.64 (s, 1 H) 7.09 (d, J = 2.64 Hz, 1 H) 7.24-7.335 (m, 2 H) 8.15 (s, 1 H) 8.49 (d, J = 1.70 Hz, 1 H) 8.95 (s, 1 H) 10.48 (br. s., 1 H) | Intermediate 376 and 5-(4,4,5,5- tetramethyl-1,3,2- dioxaborolan-2- yl)furan-2-carbocylic acid |
| Example 893 | 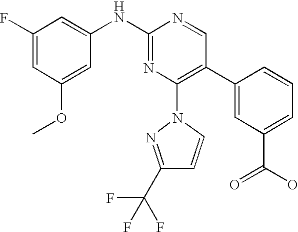 3-(2-(3-fluoro-5- methoxyphenylamino)-4-(3- (trifluoromethyl)-1H-pyrazol-1- yl)pyrimidin-5-yl)benzoic acid | MS: ES+ 474 for C₂₂H₁₅F₄N₅O₃ 1H NMR (300 MHz, DMSO-d₆) d ppm 3.77 (s, 3 H) 6.49 (dt, J = 10.93, 2.26 Hz, 1 H) 6.99 (d, J = 2.64 Hz, 1 H) 7.28-7.38 (m, 2 H) 7.39-7.53 (m, 2 H) 7.68 (s, 1 H) 7.90 (dt, J = 7.35, 1.60 Hz, 1 H) 8.37 (d, J = 1.70 Hz, 1 H) 8.81 (s, 1 H) 10.39 (s, 1 H) 12.96 (br. s., 1 H) | Intermediate 376 and 3-boronobenzoic acid |
| Example 894 | 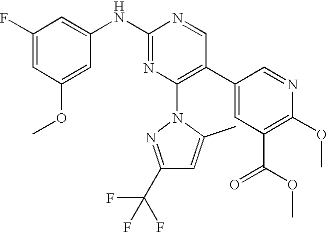 methyl 5-(2-(3-fluoro-5- methoxyphenylamino)-4-(5- methyl-3-(trifluoromethyl)-1H- pyrazol-1-yl)pyrimidin-5-yl)-2- methoxynicotinate | MS: ES+ 533 for C₂₄H₂₀F₄N₆O₄ 1H NMR (300 MHz, DMSO-d₆) d ppm 2.31 (s, 3 H) 3.75 (s, 6 H) 3.92 (s, 3 H) 6.50 (dt, J = 10.93, 2.26 Hz, 1 H) 6.76 (s, 1 H) 7.21-7.26 (m, 1 H) 7.31 (dt, J = 11.59, 1.93 Hz, 1 H) 7.62 (d, J = 2.45 Hz, 1 H) 8.23 (d, J = 2.64 Hz, 1 H) 8.97 (s, 1 H) 10.41 (s, 1 H) | Intermediate 377 and Intermediate 175 |
| Example 895 | 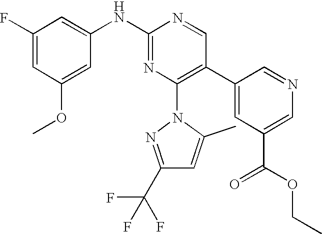 ethyl 5-(2-(3-fluoro-5- methoxyphenylamino)-4-(5- methyl-3-(trifluoromethyl)-1H- pyrazol-1-yl)pyrimidin-5- yl)nicotinate | MS: ES+ 517 for C₂₄H₂₀F₄N₆O₃ 1H NMR (300 MHz, DMSO-d₆) d ppm 1.30 (t, J = 7.06 Hz, 3 H) 2.41 (s, 3 H) 3.76 (s,3 H) 4.32 (q, J = 7.16 Hz, 2 H) 6.52 (dt, J = 10.97, 2.33 Hz, 1 H) 6.77 (s, 1 H) 7.24 (s, 1 H) 7.28- 7.37 (m, 1 H) 7.81 (t, J = 2.17 Hz, 1 H) 8.62 (d, J = 2.26 Hz, 1 H) 8.99 (d, J = 2.07 Hz, 1 H) 9.02 (s,1 H) 10.46 (s, 1 H) | Intermediate 377 and ethyl 5-(4,4,5,5- tetramethyl-1,3,2- dioxaborolan-2- yl)nicotinate |
| Example 896 | 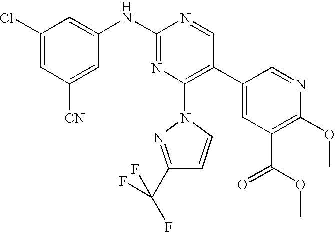 mthyl 5-(2-(3-chloro-5- cyanophenylamino)-4-(3- (trifluoromethyl)-1H-pyrazol-1- yl)pyrimidin-5-yl)-2- methoxynicotinate | MS: ES+ 530 for C₂₃H₁₅ClF₃N₇O₃ ¹H NMR (300 MHz, DMSO-d₆) d ppm 3.77 (s, 3 H) 3.95 (s, 3 H) 7.06 (d, J = 2.64 Hz, 1 H) 7.64 (t, J = 1.51 Hz, 1 H) 7.88 (d, J = 2.45 Hz, 1 H) 8.20 (d, J = 1.32 Hz, 1 H) 8.23 (t, J = 1.88 Hz, 1 H) 8.29 (d, J = 2.64 Hz, 1 H) 8.49 (d, J = 1.70 Hz, 1 H) 8.88 (s, 1 H) 10.72 (s,1 H) | Intermediate 378 and Intermediate 175 |
| Example 897 | 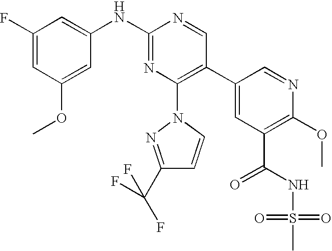 5-(2-(3-fluoro-5- methoxyphenylamino)-4-(3- (trifluoromethyl)-1H-pyrazol-1- yl)pyrimidin-5-yl)-2-methoxy-N- (methylsulfonyl)nicotinamide | MS: ES+ 582 for C₂₃H₁₉F₄N₇O₅S 1H NMR (300 MHz, DMSO-d₆) d ppm 3.33 (s, 3 H) 3.78 (s, 3 H) 3.97 (s, 3 H) 6.50 (dt, J = 10.93, 2.26 Hz, 1 H) 7.05 (d, J = 2.64 Hz, 1 H) 7.23- 7.40 (m, 2 H) 7.87 (d, J = 2.45 Hz, 1 H) 8.18 (d, 1 H) 8.46 (d, J = 1.70 Hz, 1 H) 8.80 (s, 1 H) 10.39 (s, 1 H) 11.69 (s, 1 H) | Intermediate 376 and Intermediate 368 |
| Example 898 | 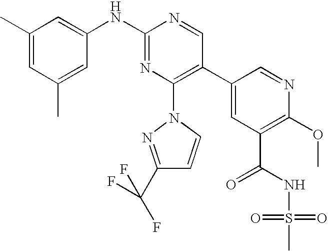 5-(2-(3,5-dimethylphenylamino)- 4-(3-(trifluoromethyl)-1H- pyrazol-1-yl)pyrimidin-5-yl)-2- methoxy-N- (methylsulfonyl)nicotinamide | MS: ES+ 562 for C₂₄H₂₂F₃N₇O₄S 1H NMR (300 MHz, DMSO-d₆) d ppm 2.27 (s, 6 H) 3.38 (s, 3 H) 4.04 (s, 3 H) 6.76 (s, 1 H) 7.05 (d, J = 2.64 Hz, 1 H) 7.44 (s, 2 H) 8.14 (d, J = 2.45 Hz, 1 H) 8.27 (s, 1 H) 8.48 (d, J = 2.45 Hz, 1 H) 8.65 (d, J = 1.70 Hz, 1 H) 8.92 (s, 1 H) 11.77 (br. s., 1 H) | Intermediate 217 and Intermediate 368 |
| Example 899 | 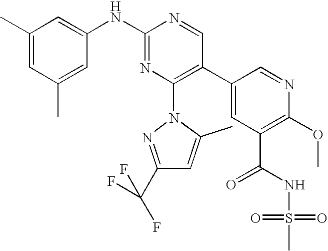 5-(2-(3,5-dimethylphenylamino)- 4-(5-methyl-3-(trifluoromethyl)- 1H-pyrazol-1-yl)pyrimidin-5-yl)- 2-methoxy-N- (methylsulfonyl)nicotinamide | MS: ES+ 576 for C₂₅H₂₄F₃N₇O₄S 1H NMR (300 MHz, DMSO-d₆) d ppm 2.25 (s, 6 H) 2.36 (s, 3 H) 3.32 (s, 3 H) 3.93 (s, 3 H) 6.70 (s, 1 H) 6.76 (s,1 H) 7.37 (s, 2 H) 7.69 (d, J = 2.45 Hz, 1 H) 8.00 (d, J = 2.64 Hz, 1 H) 8.90 (s, 1 H) 10.09 (s, 1 H) 11.70 (br. s., 1 H) | Intermediate 218 and Intermediate 368 |
| Example 900 | 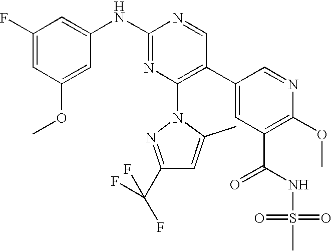 5-(2-(3-fluoro-5- methoxyphenylamino)-4-(5- methyl-3-(trifluoromethyl)-1H- pyrazol-1-yl)pyrimidin-5-yl)-2- methoxy-N- (methylsulfonyl)nicotinamide | MS: ES+ 596 for C₂₄H₂₁F₄N₇O₅S 1H NMR (300 MHz, DMSO-d₆) d ppm 2.33 (s, 3 H) 3.32 (s, 3 H) 3.75 (s, 3 H) 3.94 (s, 3 H) 6.50 (dt, J = 10.93, 2.26 Hz, 1 H) 6.77 (s, 1 H) 7.25 (s, 1 H) 7.30 (dt, J = 11.54, 1.95 Hz, 1 H) 7.70 (d, J = 2.45 hz, 1 H) 8.01 (d, J = 2.45 Hz, 1 H) 8.97 (s, 1 H) 10.42 (s, 1 H) 11.72 (s, 1 H) | Intermediate 377 and Intermediate 368 |
| Example 901 | 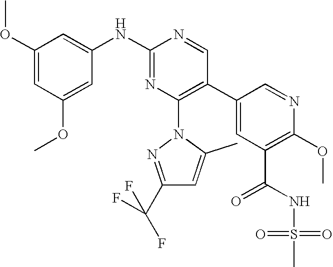 5-(2-(3,5- dimethoxyphenylamino)-4-(3- (trifluoromethyl)-1H-pyrazol-1- yl)pyrimidin-5-yl)-2-methoxy-N- (methylsulfonyl)nicotinamide | MS: ES+ 594 for C₂₄H₂₂F₃N₇O₆S ¹H NMR (300 MHz, DMSO-d₆) d ppm 33.33 (s, 3 H) 3.75 (s, 6 H) 3.97 (s, 3 H) 6.21 (t, J = 2.17 Hz,1 H) 7.03 (d, J = 2.64 Hz,1 H) 7.10 (d, J = 2.07 Hz, 2 H) 7.87 (d, J = 2.35 Hz, 1 H) 8.17 (d, J = 2.45 Hz, 1 H) 8.44 (d,J = 1.70 Hz, 1 H) 8.77 (s, 1 H) 10.17 (s, 1 H) 11.69 (br. s., 1 H) | Intermediate 215 and Intermediate 368 |
| Example 902 | 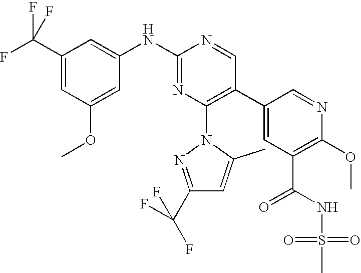 2-methoxy-5-(2-(3-methoxy-5- (trifluoromethyl)phenylamino)- 4-(5-methyl-3-(trifluoromethyl)- 1H-pyrazol-1-yl)pyrimidin-5-yl)- N-(methylsulfonyl)nicotinamide | MS: ES+ 646 for C₂₅H₂₁F₆N₇O₅S 1H NMR (300 MHz, DMSO-d₆) d ppm 2.33 (s,3 H) 3.33 (s, 3 H) 3.82 (s, 3 H) 3.94 (s, 3 H) 6.78 (s, 1 H) 6.90 (s, 1 H) 7.68 (s, 1 H) 7.73 (d, J = 2.45 Hz, 1 H) 7.82 (s,1 H) 8.01 (d, J = 2.45 Hz, 1 H) 9.01 (s, 1 H) 10.55 (s, 1 H) 11.73 (s,1 H) | Intermediate 375 and Intermediate 368 |
| Example 903 | 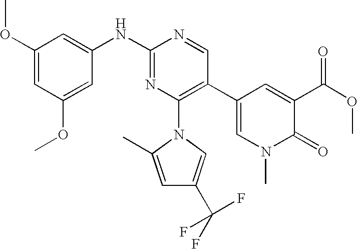 methyl 5-(2-(3,5- dimethoxyphenylamino)-4-(5- methyl-3-(trifluoromethyl)-1H- pyrazol-1-yl)pyrimidin-5-yl)-1- methyl-2-oxo-1,2- dihydropyridine-3-carboxylate | MS: ES+ 545 for C₂₅H₂₃F₃N₆O₅ 1H NMR (300 MHz, ACETONITRILE-d₃) d ppm 2.35 (s, 3 H) 3.46 (s, 3 H) 3.70 (s, 3 H) 3.75 (s, 6 H) 6.21 (t, J = 2.17 hz, 1 H) 6.56 (s, 1 H) 6.93 (d, J = 2.07 Hz, 2 H) 7.43 (d, J = 2.83 Hz, 1 H) 7.64 (d, J = 2.83 Hz, 1 H) 8.31 (s, 1 H) 8.62 (s, 1 H) | Intermediate 216 and Intermediate 369 |
| Example 904 | 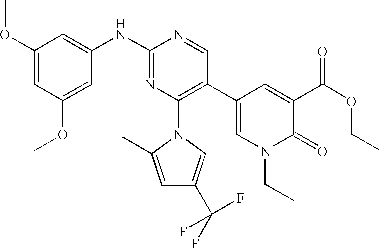 ethyl 5-(2-(3,5- dimethoxyphenylamino)-4-(5- methyl-3-(trifluoromethyl)-1H- pyrazol-1-yl)pyrimidin-5-yl)-1- ethyl-2-oxo-1,2-dihydropyridine- 3-carboxylate | MS: ES+ 573 for C₂₇H₂₇F₃N₆O₅ ¹H NMR (300 MHz, DMSO-d₆) d ppm 1.12-1.27 (m, 6 H) 2.38 (s, 3 H) 3.72 (s, 6 H) 3.94 (q, J = 6.97 Hz, 2 H) 4.13 (q, J = 7.16 Hz, 2 H) 6.21 (t, J = 2.17 Hz, 1 H) 6.79 (s, 1 H) 7.04 (d, J = 2.07 Hz, 2 H) 7.34 (d, J = 2.64 Hz, 1 H) 8.05 (d, J = 2.83 Hz, 1 H) 8.89 (s, 1 H) 10.15 (s, 1 H) | Intermediate 216 and Intermediate 370 |
| Example 905 | 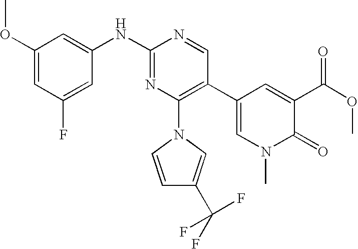 methyl 5-(2-(3-fluoro-5- methoxyphenylamino)-4-(3- (trifluoromethyl)-1H-pyrazol-1- yl)pyrimidin-5-yl)-1-methyl-2- oxo-1,2-dihydropyridine-3- carboxylate | MS: ES+ 519 for C₂₃H₁₈F₄N₆O₄ 1H NMR (300 MHz, DMSO-d₆) d ppm 3.50 (s, 3 H) 3.67 (s, 3 H) 3.77 (s, 3 H) 6.49 (dt, J = 10.93, 2.17 Hz, 1 H) 7.08 (d, J = 2.64 Hz, 1 H) 7.22- 7.36 (m, 2 H) 7.63 (d, J = 2.83 Hz, 1 H) 8.17 (d, J = 2.64 Hz, 1 H) 8.56 (d, J = 1.51 Hz, 1 H) 8.76 (s, 1 H) 10.34 (s,1 H) | Intermediate 376 and Intermediate 369 |
| Example 906 | 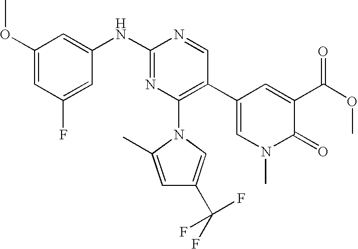 methyl 5-(2-(3-fluoro-5- ethoxyphenylamino)-4-(5- methyl-3-(trifluoromethyl)-1H- pyrazol-1-yl)pyrimidin-5-yl)-1- methyl-2-oxo-1,2- dihydropyridine-3-carboxylate | MS: ES+ 533 for C₂₄H₂₀F₄N₆O₄ 1H NMR (300 MHz, DMSO-d₆) d ppm 2.37 (s, 3 H) 3.48 (s, 3 H) 3.65 (s, 3 H) 3.75 (s, 3 H) 6.49 (dt, J = 10.93, 2.26 Hz, 1 H) 6.80 (s, 1 H) 7.17- 7.35 (m, 3 H) 8.18 (d, J = 2.83 Hz, 1 H) 8.90 (s, 1 H) 10.37 (s, 1 H) | Intermediate 377 and Intermediate 369 |
| Example 907 | 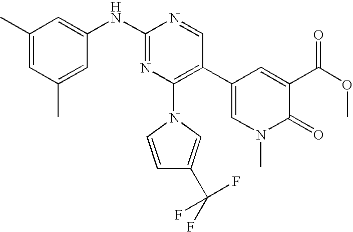 methyl 5-(2-(3,5- dimethylphenylamino)-4-(3- (trifluoromethyl)-1H-pyrazol-1- yl)pyrimidin-5-yl)-1-methyl-2- oxo-1,2-dihydropyridine-3- carboxylate | MS: ES+ 499 for C₂₄H₂₁F₃N₆O₃ 1H NMR (300 MHz, DMSO-d₆) d ppm 2.27 (s, 6 H) 3.50 (s, 3 H) 3.67 (s, 3 H) 6.69 (s, 1 H) 7.06 (d, J = 2.64 Hz, 1 H) 7.40 (s, 2 H) 7.64 (d, J = 2.64 Hz, 1 H) 8.15 (d, J = 2.64 Hz, 1 H) 8.54 (d, J = 1.70 Hz, 1 H) 8.69 (s, 1 H) 10.01 (s, 1 H) | Intermediate 217 and Intermediate 369 |
| Example 908 | 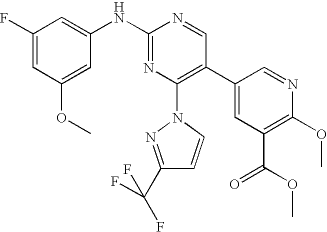 methyl 5-(2-(3-fluoro-5- methoxyphenylamino)-4-(3- (trifluoromethyl)-1H-oyrazol-1- yl)pyrimidin-5-yl)-2- methoxynicotinate | MS: ES+ 519 for C₂₃H₁₈F₄N₆O₄ 1H NMR (300 MHz, DMSO-d₆) d ppm 3.77 (s,3 H) 3.77 (s, 3 H) 3.95 (s, 3 H) 6.49 (dt, J = 10.93, 2.26 Hz, 1 H) 7.04 (d, J = 2.64 Hz, 1 H) 7.28- 7.36 (m, 2 H) 7.86 (d, J = 2.45 Hz, 1 H) 8.49 (dd, J = 2.64, 0.94 Hz, 1 H) 8.81 (s, 1 H) 10.37 (s,1 H) | Intermediate 376 and Intermediate 175 |
| Example 909 | 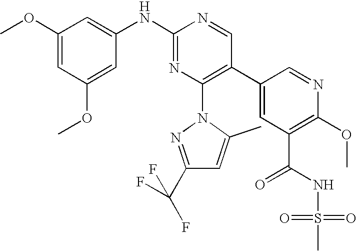 5-(2-(3,5- dimethoxyphenylamino)-4-(5- methyl-3-(trifluoromethyl)-1H- pyrazol-1-yl)pyrimidin-5-yl)-2- methoxy-N- (methylsulfonyl)nicotinamide | MS: ES+ 608 for C₂₅H₂₄F₃N₇O₆S\ 1H NMR (300 MHz, DMSO-d₆) d ppm 2.31 (s, 3 H) 3.32 (s, 3 H) 3.72 (s, 6 H) 3.93 (s, 3 H) 6.21 (t, J = 2.17 Hz, 1 H) 6.76 (s, 1 H) 7.05 (d, J = 2.26 Hz, 2 H) 7.71 (d, J = 2.45 Hz, 1 H) 7.99 (d, J = 2.45 Hz, 1 H) 8.93 (s, 1 H) 10.20 (s, 1 H) 11.82 (br. s., 1 H) | Intermediate 216 and Intermediate 368 |

The following example was prepared using the general method described above for Example 212 and the specified starting materials.

| example | compound | mass and nmr data | sm |
|---|---|---|---|
| Example 910 | 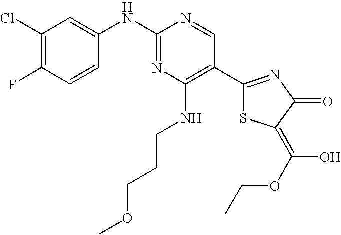 (Z)-2-(2-(3-chloro-4- fluorophenylamino)-4-(3- methoxypropylamino)- pyrimidin-5-yl)-5- (ethoxy(hydroxy)methylene)- thiazol-4(5H)-one | MS: ES+ 482 for C₂₀H₂₁ClFN₅O₄S 1H NMR (300 MHz, DMSO-d₆) d ppm 1.26 (t, 3 H) 1.83-1.97 (m, J = 6.66, 6.66, 6.55, 6.31 Hz, 2 H) 3.25 (s, 3H) 3.44 (t, J = 6.12 Hz, 2 H) 3.60 (q, J = 6.72 Hz, 2 H) 4.21 (q, J = 6.97 Hz, 2 H) 7.35 (t, J = 9.14 Hz, 1 H) 7.54-7.69 (m, 1 H) 8.21 (dd, J = 6.69, 2.35 Hz, 1 H) 8.58 (s, 1 H) 9.20 (t, J = 4.90 Hz, 1 H) 10.01 (br. s., 1 H) 12.04 (br.s., 1 H) | Intermediate 128 and diethyl chloro malonate |

The compounds in the below table were prepared using the same procedure as Example 214, using the starting material and base specified.

| example | compound | mass and nmr data | sm |
|---|---|---|---|
| Example 911 | 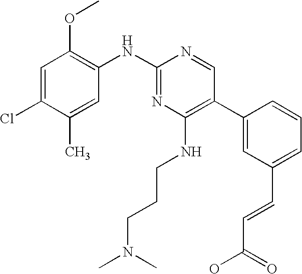 (E)-3-(3-(2-(4-chloro-2- methoxy-5-methyl- phenylamino)-4-(3- (dimethylamino)propyl amino)pyrimidin-5- yl)phenyl)acrylic acid | MS: ES+ 496 for C₂₆H₃₀ClN₅O₃ 1H NMR (300 MHz, DMSO- d₆) δ ppm 1.93 (quin, J = 7.02 Hz, 2 H) 2.31 (s,3 H) 2.70 (d, J = 4.71 Hz, 6 H) 2.92-3.06 (m, 2 H) 3.36-3.50 (m, 2 H) 3.86 (s, 3 H) 6.62 (d, J = 16.01 Hz, 1 H) 7.22 (s, 1 H) 7.43-7.50 (m, 1 H) 7.56 (t, J = 7.63 Hz, 1 H) 7.64 (d, J = 16.01 Hz, 1 H) 7.73- 7.82 (m, 2 H) 7.86 (s, 2 H) 8.07 (br. s., 1 H) 9.57 (br. s., 1 H) 10.10 (br. s., 1 H) 12.51 (br. s., 1 H) | Example 343 and sodium hydroxide |
| Example 912 | 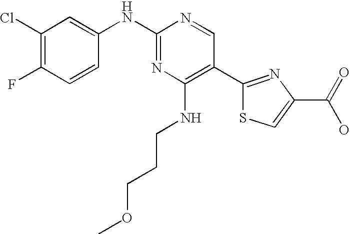 2-(2-(3-chloro-4-fluoro- phenylamino)-4-(3- methoxypropylamino) pyrimidin-5-yl)thiazol-4- carboxylic acid | MS: ES+ 438 for C₁₈H₁₇ClFN₅O₃S 1H NMR (300 MHz, DMSO- d₆) δ ppm 1.88 (qd, J = 6.59, 6.40 Hz, 2 H) 3.25 (s, 3 H) 3.47 (t, J = 6.03 Hz, 2 H) 3.60 (q, J = 6.34 Hz, 2 H) 7.30 (t, 1 H) 7.56-7.69 (m, 1 H) 7.83 (s, 1 H) 8.24 (dd, J = 6.78, 1.88 Hz, 1 H) 8.47 (s, 1 H) 9.74 (br. s., 1 H) 9.85 (t, J = 4.14 Hz, 1 H) | Example 212 and barium hydroxide |
| Example 913 | 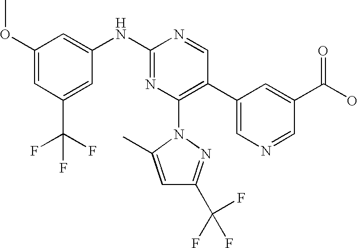 5-(2-(3-methoxy-5- (trifluoromethyl)phenylamino)- 4-(5-methyl-3- (trifluoromethyl)-1H-pyrazol- 1-yl)pyrimidin-5-yl)nicotinic acid | MS: ES+ 539 for C₂₃H₁₆F6N₆O₃ 1H NMR (300 MHz, DMSO- d₆) δ ppm 2.43 (s, 3 H) 3.83 (s, 3 H) 6.78 (s, 1 H) 6.92 (s, 1 H) 7.69 (s, 1 H) 7.82 (s, 1 H) 7.86 (t, J = 2.17 Hz, 1 H) 8.56 (d, J = 2.26 Hz, 1 H) 8.97 (d, J = 1.88 Hz, 1 H) 9.03 (s, 1 H) 10.57 (s, 1 H) | Example 888 and sodium hydroxide |
| Example 914 | 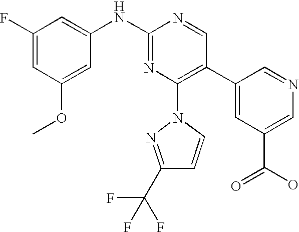 5-(2-(3-fluoro-5- methoxyphenylamino)-4-(3- (trifluoromthyl)-1H-pyrazol- 1-yl)pyrimidin-5-yl)nicotinic acid | MS: ES+ 475 for C₂₁H₁₄F₄N₆O₃ 1H NMR (300 MHz, DMSO- d₆) d ppm 3.78 (s, 3 H) 6.51 (dt, J = 10.93, 2.17 Hz, 1 H) 7.04 (d, J = 2.64 Hz, 1 H) 7.26-7.39 (m, 2 H) 8.03 (t, J = 2.07 Hz, 1 H) 8.54 (d, J = 1.51 Hz, 1 H) 8.65 (d, J = 2.26 Hz, 1 H) 8.85 (s, 1 H) 9.01 (d, J = 2.07 Hz, 1 H) 10.42 (s, 1 H) 13.37 (br. s., 1 H) | Example 889 and sodium hydroxide |
| Example 915 | 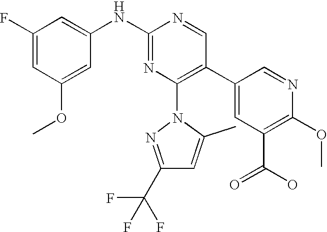 5-(2-(3-fluoro-5- methoxyphenylamino)-4-(5- methyl-3-(trifluoromethyl)- 1H-pyrazol-1-yl)pyrimidin-5- yl)-2-methoxynicotinic acid | MS: ES+ 519 for C₂₃H₁₈F₄N₆O₄ 1H NMR (300 MHz, DMSO- d₆) d ppom 2.33 (s, 3 H) 3.75 (s, 3 H) 3.91 (s, 3 H) 6.50 (dt, J = 10.93, 2.26 Hz, 1 H) 6.75 (s, 1 H) 7.24 (s, 1 H) 7.31 (dt, J = 11.49, 1.88 Hz, 1 H) 7.64 (d, J = 2.64 Hz, 1 H) 8.16 (d, J = 2.64 Hz, 1 H) 8.96 (s, 1 H) 10.39 (s, 1 H) 12.94 (br. s., 1 H) | Example 894 and sodium hydroxide |
| Example 916 | 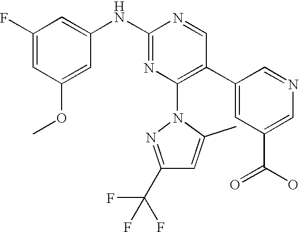 5-(2-(3-fluoro-5- methoxyphenylamino)-4-(5- methyl-3-(trifluoromethyl)- 1H-pyrazol-1-yl)pyrimidin-5- yl)nicotinic acid | MS: ES+ 489 for C₂₂H₁₆F₄N₆O₃ 1H NMR (300 MHz, DMSO- d₆) d ppm 2.42 (s, 3 H) 3.76 (s, 3 H) 6.52 (dt, J = 10.93, 2.17 Hz, 1 H) 6.77 (s, 1 H) 7.24 (s, 1 H) 7.32 (d, J = 11.340 Hz, 1 H) 7.84 (t, J = 2.07 Hz, 1 H) 8.55 (d, J = 2.07 Hz, 1 H) 8.97 (d, J = 1.88 Hz, 1 H) 9.01 (s, 1 H) 10.44 (s, 1 H) 13.42 (br. s., 1 H) | Example 895 and sodium hydroxide |
| Example 917 | 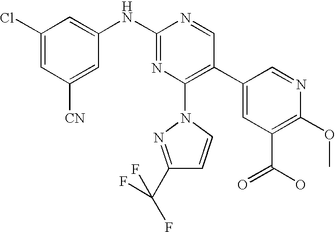 5-(2-(3-chloro-5- cyanophenylamino)-4-(3- (trifluoromethyl)-1H-pyrazol- 1-yl)pyrimidin-5-yl)-2- methoxynicotinic acid | MS: ES+ 516 for C₂₂H₁₃ClF₃N₇O₃ 1H NMR (300 MHz, CHLOROFORM-d) d ppm 4.27 (s, 3 H) 6.72 (d, J = 2.643 Hz, 1 H) 7.39 (d, J = 1.32 Hz, 1 H) 7.86 (br. s., 1 H) 7.94-8.07 (m, 2 H) 8.25-8.31 (m, 1 H) 8.34 (d, J = 2.45 Hz, 1 H) 8.47 (br. s., 1 H) 8.55 (br. s., 1 H) | Example 896 and sodium hydroxide |
| Example 918 | 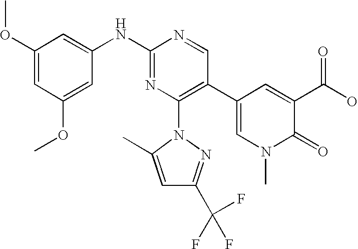 5-(2-(3,5- dimethoxyphenylamino)-4- (5-methyl-3- (trifluoromnethyl)-1H-pyrazol- 1-yl)poyrimidin-5-yl)-1- methyl-2-oxo-1,2- dihydropyridine-3-carboxylic acid | MS: ES+ 531 for C₂₄H₂₁F₃N₆O₅ 1H NMR (300 MHz, DMSO- d₆) d ppm 2.48 (s, 3 H) 3.67 (s, 3 H) 3.73 (s,6 H) 6.22 (t, J = 2.07 Hz, 1 H) 6.80 (s, 1 H) 7.03 (d, J = 2.26 Hz, 2 H) 7.65 (d, J = 2.64 Hz,1 H) 8.41 (d, J = 2.64 Hz, 1 H) 8.86 (s, 1 H) 10.17 (s,1 H) 14.37 (br. s., 1 H) | Example 903 and sodium hydroxide |
| Example 919 | 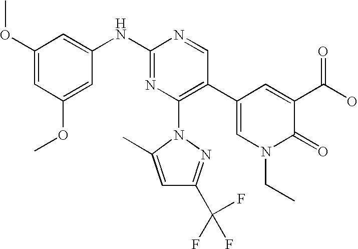 5-(2-(3,5- dimethoxyphenylamino)-4- (5-methyl-3- (trifluoromethyl)-1H-pyrazol- 1-yl)pyrimidin-5-yl)-1-ethyl- 2-oxo-1,2-dihydropyridine-3- carboxylic acid | MS: ES+ 545 for C₂₅H₂₃F₃N₆O₅ 1H NMR (3300 MHz, DMSO- d₆) d ppm 1.28 (t, 3 H) 2.48 (s, 3 H) 3.73 (s,6 H) 4.11 (q, J = 7.16 Hz, 2 H) 6.22 (t, J = 2.26 Hz, 1 H) 6.79 (s, 1 H) 7.03 (d, J = 2.26 Hz, 2 H) 7.81 (d, J = 2.64 Hz, 1 H) 8.29 (d, J = 2.64 Hz, 1 H) 8.90 (s, 1 H) 10.17 (s, 1 H) 14.42 (br. s., 1 H) | Example 904 and sodium hydroxide |
| Example 920 | 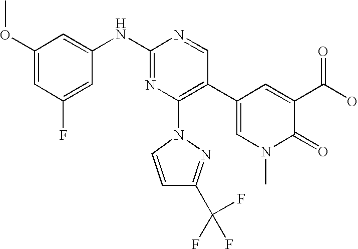 5-(2-(3-fluoro-5- methoxyphenylamino)-4-(3- (trifluoromethyl)-1H-pyrazol- 1-yl)pyrimidin-5-yl)-1- methyl-2-oxo-1,2- dihydropyridine-3-carboxylic acid | MS: ES+ 505 for C₂₂H₁₆F₄N₆O₄ 1H NMR (300 MHz, DMSO- d₆) d ppm 3.70 (s, 3 H) 3.78 (s, 3 H) 6.51 (dt, 1 H) 7.09 (d, J = 2.64 Hz, 1 H) 7.23-7.36 (m, 2 H) 8.05 (d, J = 2.64 Hz, 1 H) 8.43 (d, J = 2.45 Hz, 1 H) 8.60 (d, J = 1.70 Hz, 1 H) 8.76 (s, 1 H) 10.38 (s, 1 H) 14.48 (br. s., 1 H) | Example 905 and sodium hydroxide |
| Example 921 | 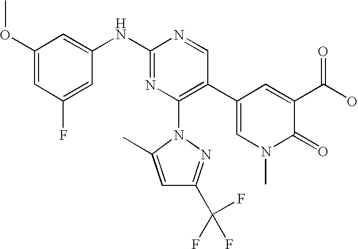 5-(2-(3-fluoro-5- methoxyphenylamino)-4-(5- methyl-3-(trifluoromethyl)- 1H-pyrazol-1-yl)pyrimidin-5- yl)-1-methyl-2-oxo-1,2- dihydropyridine-3-carboxylic acid | MS: Es+ 519 for C₂₅H₁₈F₄N₆O₄ 1H NMR (300 MHz, DMSO- d₆) d ppm 22.49 (br. s., 3 H) 3.68 (s, 3 H) 3.76 (s, 3 H) 6.51 (dt, J = 10.93, 2.07 Hz, 1 H) 6.82 (s, 1 H) 7.23 (s, 1 H) 7.30 (d, J = 11.68 Hz, 1 H) 7.66 (d, J = 2.64 hz, 1 H) 8.42 (d, J = 2.64 Hz, 1 H) 8.91 (s, 1 H) 10.39 (s, 1 H) 14.36 (br. s., 1 H) | Example 906 and sodium hydroxide |
| Example 922 | 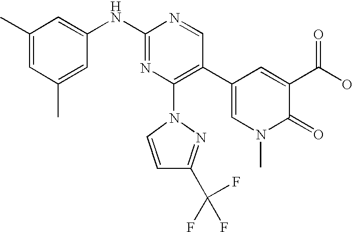 5-(2-(3,5- dimethylphenylamino)-4-(3- (trifluoromethyl)-1H-pyrazol- 1-yl)pyrimidin-5-yl)-1- methyl-2-oxo-1,2- dihydropyridine-3-carboxylic acid | MS: ES+ 485 for C₂₃H₁₉F₃N₆O₃ 1H NMR (300 MHz, DMSO- d₆) d ppm 2.28 (s, 6 H) 3.70 (s, 3 H) 6.70 (s, 1 H) 7.07 (d, J = 2.64 Hz, 1 H) 7.40 (s, 2 H) 8.06 (d, J = 2.64 Hz, 1 H) 8.41 (d, J = 2.64 Hz, 1 H) 8.58 (dd, J = 2.64, 0.94 Hz, 1 H) 8.69 (s, 1 H) 10.06 (s, 1 H) 14.51 (s, 1 | Example 907 and sodium hydroxide |
| Example 923 | 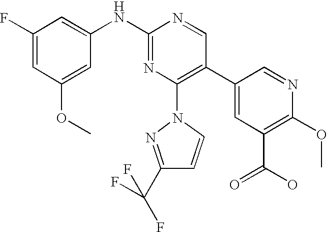 5-(2-(3-fluoro-5- methoxyphenylamino)-4-(3- (trifluoromethyl)-1H-pyrazol- 1-yl)pyrimidin-5-yl)-2- methoxynicotinic acid | MS: ES+ 505 for C₂₂H₁₆F₄N₆O₄ 1H NMR (300 MHz, DMSO- d₆) d ppm 3.77 (s, 3 H) 3.92 (s, 3 H) 6.49 (dt, J = 10.93, 2.17 Hz, 1 H) 7.02 (d, J = 2.83 Hz, 1 H) 7.31 (s, 1 H) 7.34 (t, J = 1.98 Hz, 1 H) 7.79 (d, J = 2.45 Hz, 1 H) 8.18 (d, J = 2.45 Hz, 1 H) 8.45 (d, J = 1.70 Hz, 1 H) 8.79 (s, 1 H) 10.36 (s, 1 H) 12.98 (br. s., 1 H) | Example 908 and sodium hydroxide |

The compounds in the table below were prepared using the same procedure as Example 360 with the specified starting materials.

| example | compound | mass and NMR data | sm |
|---|---|---|---|
| Example 924 | 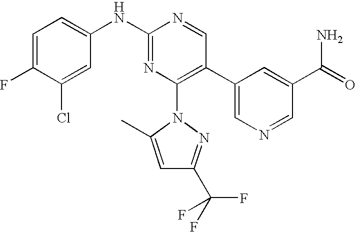 5-(2-(3-chloro-4- fluorophenylamino)-4-(5- methyl-3-(trifluoromethyl)- 1H-pyrazol-1-yl)pyrimidin-5- yl)nicotinamide | MS: ES+ 492 for C₂₁H₁₄ClF₄N₇O 1H NMR (300 MHz, DMSO-d₆) d ppm 2.43 (s, 3 H) 6.78 (s, 1 H) 7.43 (t, J = 9.14 Hz, 1 H) 7.56- 7.73 (m, 2 H) 7.98 (t, J = 1.98 Hz, 1 H) 8.07 (dd, J = 6.78, 2.26 Hz, 1 H) 8.13 (s, 1 H) 8.27 (d, J = 2.07 Hz, 1 H) 8.91 (d, J = 1.88 Hz, 1H) 8.99 (s, 1 H) 10.49 (s, 1 H) | Example 319 and amonia |
| Example 925 | 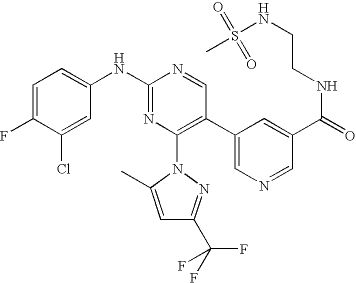 5-(2-(3-chloro-4- fluorophenylamino)-4-(5- methyl-3-(trifluoromethyl)- 1H-pyrazol-1-yl)pyrimidin-5- yl)-N-(2- (methylsulfonamido)ethyl)- nictinamide | MS: ES+ 613 for C₂₄H₂₁ClF₄N₈O₃S 1H NMR (300 MHz, DMSO-d₆) d ppm 2.43 (s, 3 H) 2.91 (s, 3 H) 3.12 (q, J = 6.22 Hz, 2 H) 3.39 (q, J = 6.22 Hz, 2 H) 6.79 (s, 1 H) 7.20 (t, J = 5.93 Hz, 1 H) 7.43 (t, J = 9.04 Hz, 1 H) 7.66 (ddd, J = 9.04, 4.14, 2.83 Hz, 1 H) 7.98- 8.12 (m, 2 H) 8.23 (d, J = 2.07 Hz, 1 H) 8.77 (t, J = 5.56 Hz, 1 H) 8.90 (d, J = 1.88 Hz, 1 H) 8.99 (s, 1 H) 10.51 (s, 1 H) | Example 319 and N-(2- aminoethyl) methane- sulfonamide |
| Example 926 | 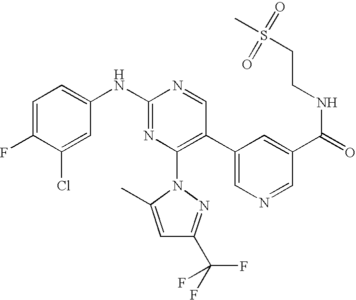 5-(2-(3-chloro-4- fluorophenylamino)-4-(5- methyl-3-(trifluoromethyl)- 1H-pyrazol-1-yl)pyrimidin-5- yl)-N-(2- (methylsulfonyl)ethyl)- nicotinamide | MS: ES+ 598 for C₂₄H₂₀ClF₄N₇O₃S 1H NMR (300 MHz, DMSO-d₆) d ppm 2.44 (s, 3 H) 3.04 (s, 3 H) 3.37-3.43 (m, 2 H) 3.69 (q, J = 6.53 Hz, 2 H) 6.79 (s, 1 H) 7.43 (t, J = 9.14 Hz, 1 H) 7.66 (ddd, J = 9.14, 4.14, 2.73 Hz, 1 H) 7.99 (t, J = 2.07 Hz, 1 H) 8.07 (dd, J = 6.78, 2.45 Hz, 1 H) 8.25 (d, J = 2.07 Hz, 1 H) 8.88 (d, J = 1.88 Hz, 1 H) 8.92-9.02 (m, 2 H) 10.50 (s, 1 H) | Example 319 and 2- aminoethyl methyl sulfone hydro chloride |
| Example 927 | 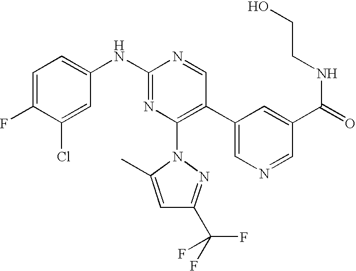 5-(2-(3-chloro-4- fluorophenylamino)-4-(5- methyl-3-(trifluoromethyl)- 1H-pyrazol-1-yl)pyrimidin-5- yl)-N-(2- hydroxyethyl)nicotinamide | MS: ES+ 536 for C₂₃H₁₈ClF₄N₇O₂ 1H NMR (300 MHz, DMSO-d₆) d ppm 2.43 (s, 3 H) 3.34 (q, J = 5.90 Hz, 2 H) 3.47-3.55 (m,2 H) 6.78 (s, 1 H) 7.43 (t, J = 9.04 Hz, 1 H) 7.66 (ddd, J = 8.90, 4.00, 2.73 Hz, 1 H) 8.01-8.13 (m, 2 H) 8.21 (d, J = 2.07 Hz, 1 H) 8.67 (t, J = 5.56 Hz, 1 H) 8.90 (d,1 H) 9.00 (s, 1 H) 10.50 (s, 1 H) | Example 319 and ethanolamine |
| Example 928 | 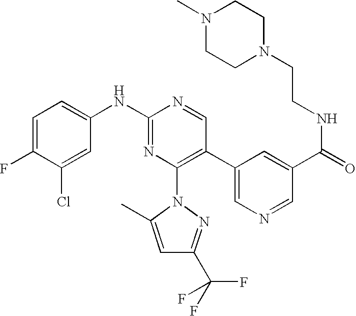 5-(2-(3-chloro-4- fluorophenylamino)-4-(5- methyl-3-(trifluoromethyl)- 1H-pyrazol-1-yl)pyrimidin-5- yl)-N-(2-(4-methylpiperazin- 1-yl)ethyl)nicotinamide | MS: ES+ 618 for C₂₈H₂₈ClF₄N₉O 1H NMR (300 MHz, DMSO-d₆) d ppm 2.45 (s, 3 H) 2.73-3.75 (m, 15 H) 6.79 (s, 1 H) 7.44 (t, J = 9.04 Hz, 1 H) 7.65 (ddd, J = 9.00, 4.10, 2.92 Hz, 1 H) 8.02 (t, J = 1.98 Hz, 1 H) 8.08 (dd, J = 6.69, 2.17 Hz, 1 H) 8.25 (d, J = 2.07 Hz, 1 H) 8.86 (t, J = 5.27 Hz, 1 H) 8.90 (d, J = 1.88 Hz, 1 H) 8.97 (s, 1 H) 10.51 (s, 1 H) | Example 319 and 2-(4- methyl- piperazin- 1-yl)-ethyl amine |
| Example 929 | 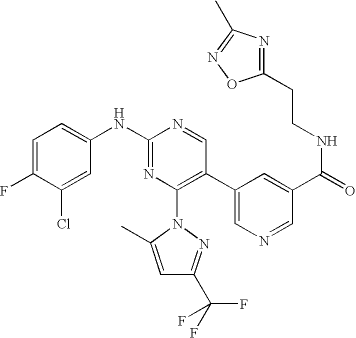 5-(2-(3-chloro-4- fluorophenylamino)-4-(5- methyl-3-(trifluoromethyl)- 1H-pyrazol-1-yl)pyrimidin-5- yl)-N-(2-(3-methyl-1,2,4- oxadiazol-5- yl)ethyl)nicotinamide | MS: ES+ 602 for C₂₆H₂₀ClF₄N₉O₂ 1H NMR (300 MHz, DMSO-d₆) d ppm 2.31 (s, 3 H) 2.43 (s, 3 H) 3.16 (t, J = 6.78 Hz, 2 H) 3.67 (q, J = 6.47 Hz, 2 H) 6.78 (s, 1 H) 7.43 (t, J = 9.04 Hz, 1 H) 7.66 (ddd, J = 9.04, 4.14, 2.83 Hz, 1 H) 7.96 (t, J = 1.98 Hz, 1 H) 8.07 (dd, J = 6.69, 2.35 Hz, 1 H) 8.25 (d, J = 2.07 Hz, 1 H) 8.85 (d, J = 1.88 Hz, 1 H) 8.91 (t, J = 5.46 Hz, 1 H) 8.98 (s, 1 H) 10.50 (s, 1 H) | Example 319 and 2-(3- methyl-1,2,4- oxadiazol- 5-yl)ethan amine, HCl |
| Example 930 | 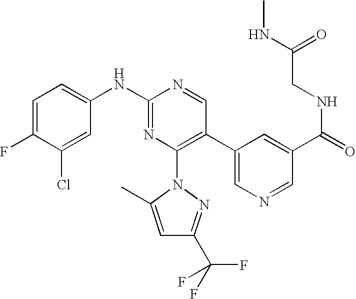 5-(2-(3-chloro-4- fluorophenylamino)-4-(5- methyl-3-(trifluoromethyl)- 1H-pyrazol-1-yl)pyrimidin-5- yl)-N-(2-(methylamino)-2- oxoethyl)nicotinamide | MS: ES+ 563 for C₂₄H₁₉ClF₄N₈O₂ 1H NMR (300 MHz, DMSO-d₆) d ppm 2.44 (s, 3 H) 2.61 (d, J = 4.33 Hz, 3 H) 3.85 (d, J = 5.65 Hz, 2 H) 6.79 (s, 1 H) 7.43 (t, J = 9.14 Hz, 1 H) 7.60-7.71 (m, 1 H) 7.86-7.95 (m, 1 H) 8.07 (dd, 1 H) 8.11-8.15 (m, 1 H) 8.19 (d, J = 1.88 Hz, 1 H) 8.93 (d, J = 1.70 Hz, 1 H) 8.97-9.05 (m,2 H) 10.51 (s, 1 H) | Example 319 and 2-amino- N-methyl acetamide, HCl |
| Example 931 | 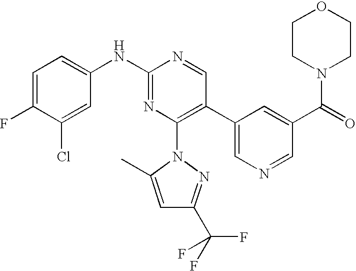 (5-(2-(3-chloro-4- fluorophenylamino)-4-(5- methyl-3-(trifluoromethyl)- 1H-pyrazol-1-yl)pyrimidin-5- yl)pyridin-3- yl)(morpholino)methanone | MS: ES+ 562 for C₂₅H₂₀ClF₄N₇O₂ 1H NMR (300 MHz, DMSO-d₆) d ppm 2.42 (br. s., 2 H) 3.11 (br. s., 2 H) 3.27 (s, 3 H) 3.54 (br. s., 4 H) 6.73 (s, 1 H) 7.36 (t, J = 9.04 Hz, 1 H) 7.44-7.51 (m, 1 H) 7.59 (dd, J = 5.18, 3.11 Hz, 1 H) 8.00 (dd, J= 6.59, 2.07 Hz, 1 H) 8.36 (d, J = 1.88 Hz, 1 H) 8.48 (d, J = 1.70 Hz, 1 H) 8.87 (s, 1 H) 10.40 (s, 1 H) | Example 319 and morpholine |
| Example 932 | 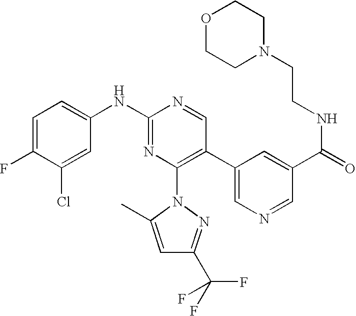 5-(2-(3-chloro-4- fluorophenylamino)-4-(5- methyl-3-(trifluoromethyl)- 1H-pyrazol-1-yl)pyrimidin-5- yl)-N-(2-morpholinoethyl) nicotinamide | MS: ES+ 605 for C₂₇H₂₅ClF₄N₈O₂ 1H NMR (300 MHz, DMSO-d₆) d ppm 2.37 (s, 7 H) 3.29-3.42 (m, 4 H) 3.52 (t, J = 3.96 Hz, 4 H) 6.71 (s, 1 H) 7.37 (t, J = 9.14 Hz, 1 H) 7.59 (dt, J = 8.85, 3.49 Hz, 1 H) 7.90 (s, 1 H) 8.01 (dd, J = 6.59, 2.07 Hz, 1 H) 8.19 (d, J = 1.88 Hz, 1 H) 8.58 (br. s., 1 H) 8.81 (d, J = 1.51 Hz, 1 H) 8.93 (s, 1 H) 10.43 (s, 1 H) | Example 319 and 2- morpholino ethylamine |
| Example 933 | 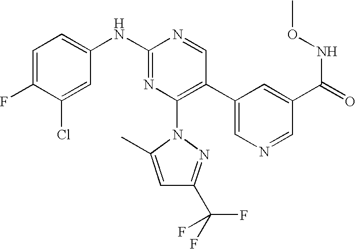 5-(2-(3-chloro-4- fluorophenylamino)-4-(5- methyl-3-(trifluoromethyl)- 1H-pyrazol-1-yl)pyrimidin-5- yl)-N-methoxynicotinamide | MS: ES+ 522 for C₂₂H₁₆ClF₄N₇O₂ 1H NMR (300 MHz, DMSO-d₆) d ppm 2.43 (s, 3 H) 3.71 (s, 3 H) 6.79 (s, 1 H) 7.43 (t, J = 9.04 Hz, 1 H) 7.66 (dt, J = 8.81, 3.41 Hz, 1 H) 7.84 (s, 1 H) 8.07 (dd, J = 6.59, 2.26 Hz, 1 H) 8.32 (d, J = 1.88 Hz, 1 H) 8.79 (d, J = 1.70 Hz, 1 H) 8.99 (s,1 H) 10.50 (s, 1 H) 11.91 (br. s., 1 H) | Example 319 and methoxyl amine hydro chloride |
| Example 934 | 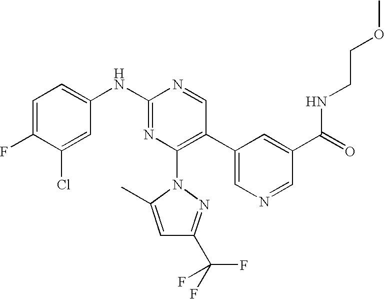 5-(2-(3-chloro-4- fluorophenylamino)-4-(5- methyl-3-(trifluoromethyl)- 1H-pyrazol-1-yl)pyrimidin-5- yl)-N-(2- methoxyethyl)nicotinamide | MS: ES+ 550 for C₂₄H₂₀ClF₄N₇O₂ 1H NMR (300 MHz, DMSO-d₆) d ppm 2.43 (s, 3 H) 3.27 (s, 3 H) 3.40-3.50 (m, 4 H) 6.78 (s, 1 H) 7.43 (t, J = 9.04 Hz, 1 H) 7.66 (dt, J = 8.85, 3.48 Hz, 1 H) 8.00 (s, 1 H) 8.08 (dd, J = 6.50, 1.98 Hz, 1 H) 8.23 (d, J = 1.88 Hz, 1 H) 8.69- 8.80 (m, 1 H) 8.89 (d, J = 1.70 Hz, 1 H) 9.00 (s, 1 H) 10.50 (s, 1 H) | Example 319 and 2- methoxy ethylamine |
| Example 935 | 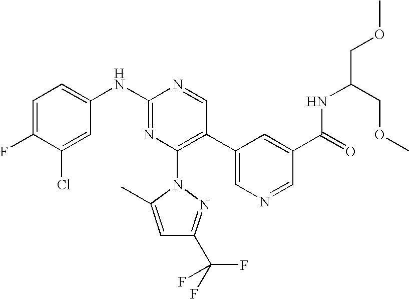 5-(2-(3-chloro-4- fluorophenylamino)-4-(5- methyl-3-(trifluoromethyl)- 1H-pyrazol-1-yl)pyrimidin-5- yl)-N-(1,3-dimethoxypropan- 2-yl)nicotinamide | MS: ES+ 594 for C₂₆H₂₄ClF₄N₇O₃ 1H NMR (300 MHz, DMSO-d₆) d ppm 2.44 (s, 3 H) 3.27 (s, 6 H) 3.45 (dd, J = 5.84, 2.26 Hz, 4 H) 4.30 (dt, 1 H) 6.78 (s, 1 H) 7.43 (t, J = 9.14 Hz, 1 H) 7.66 (ddd, J = 9.04, 4.24, 2.73 Hz, 1 H) 8.04 (t, J= 2.07 Hz, 1 H) 8.08 (dd, J = 6.59, 2.26 Hz, 1 H) 8.25 (d, J = 2.07 Hz, 1 H) 8.53 (d, J = 8.10 Hz,1 H) 8.90 (d, J = 2.07 Hz, 1 H) 9.02 (s, 1 H) 10.50 (s, 1 H) | Example 319 and 1,3- dimethoxy propan-2- amine |
| Example 936 | 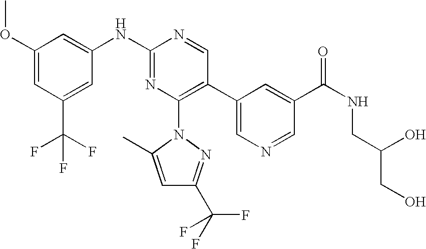 N-(2,3-dihydroxypropyl)-5- (2-(3-methoxy-5- (trifluoromethyl)phenyl- amino)-4-(5-methyl-3- (trifluoromethyl)-1H- pyrazol-1-yl)pyrimidin-5- yl)nicotinamide | MS: ES+ 612 for C₂₆H₂₃F₆N₇O₄ 1H NMR (300 MHz, DMSO-d₆) d ppm 2.41 (s, 3 H) 3.14-3.26 (m, 1 H) 3.33-3.48 (m, 3 H) 3.58- 3.70 (m, 1 H) 3.83 (s, 3 H) 4.55 (t, J = 5.75 Hz, 1 H) 4.81 (d, J = 4.90 Hz, 1 H) 6.78 (s, 1 H) 6.92(s, 1 H) 7.68 (s, 1 H) 7.84 (s, 1 H) 8.08 (t, J = 2.07 Hz, 1 H) 8.20 (d, J = 2.07 Hz, 1 H) 8.60 (t, J = 5.56 Hz, 1 H) 8.90 (d, J = 2.07 Hz, 1 H) 9.04 (s, 1 H) 10.59 (s, 1 H) | Example 913 and 3- amino-1,2- propanediol |

### Example 937

### N2-(3-chloro-4-fluorophenyl)-N4-(3-methoxypropyl)-5-(1H-tetrazol-5-yl)pyrimidine-2,4-diamine

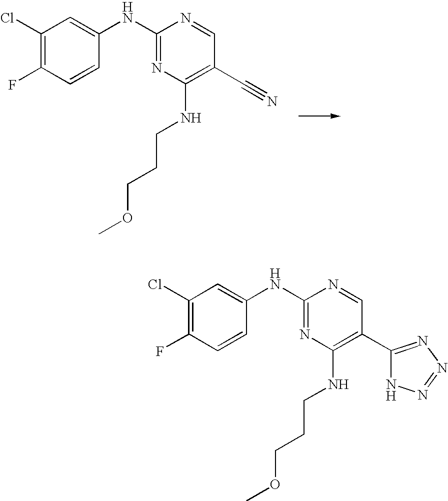

Intermediate 127 (100 mg, 0.30 mmol), sodium azide (77 mg, 1.2 mmol), and ammonium chloride (64 mg, 1.2 mmol) were combined in N,N-dimethylformamide (2 mL) and heated at 100 degrees C. for 3 hours. The reaction mixture was allowed to cool, then it was filtered. Reverse-phase chromatography (C18: water, acetonitrile, formic acid additive) was used to isolate the desired product (59 mg).

MS: ES+ 379 for C₁₅H₁₆ClFN₈O.

1H NMR (300 MHz, DMSO-d₆) δ ppm 1.90 (quin, J=6.45 Hz, 2H) 3.25 (s, 3H) 3.46 (t, J=6.03 Hz, 2H) 3.64 (q, J=6.53 Hz, 2H) 7.34 (t, J=9.14 Hz, 1H) 7.58-7.73 (m, 1H), 8.20 (dd, J=6.78, 2.45 Hz, 1H) 8.48 (t, J=4.80 Hz, 1H) 8.63 (s, 1H) 9.88 (s, 1H).

The compound in the table below was made using the method above for Example 937 with the specified starting material.

| example | compound | mass and nmr data | sm |
|---|---|---|---|
| Example 938 | 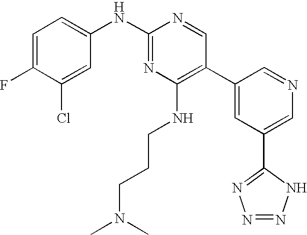 5-(5-(1H-tetrazol-5- yl)pyridin-3-yl)-N2-(3- chloro-4-fluorophenyl)-N4- (3- (dimethylamino)propyl)- pyrimidin-2,4-diamine | MS: ES+ 469 for C₂₁H₂₂ClF₄N₇O 1H NMR (300 MHz, DMSO-d₆) d ppm 1.59-1.73 (m, 2 H) 1.92 (s, 6 H) 2.27 (t, J = 6.31 Hz, 2 H) 3.38- 3.50 (m, 2 H) 7.30 (t, J = 9.14 Hz, 1 H) 7.48 (t, J = 4.71 Hz, 1 H) 7.65 (ddd, J = 9.09, 4.10, 2.83 Hz, 1 H) 7.84 (s, 1 H) 8.20 (t, J = 2.07 Hz, 1 H) 8.26 (dd, J = 6.88, 2.54 Hz, 1 H) 8.43 (d, J = 2.26 Hz, 1 H) 9.12 (d, J = 2.07 Hz, 1 H) 9.44 (s, 1 H) | Example 15 |

The compounds in the below table were prepared using the general method described above for Example 1 and the starting materials (SM) indicated.

| Ex | Compound | Data | SM |
|---|---|---|---|
| Example 939 | 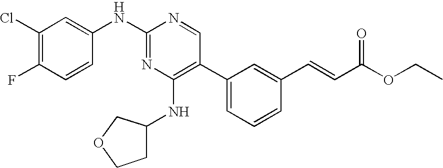 (E)-ethyl 3-(3- (2-(3-chloro-4- fluorophenyl- amino)-4- (tetrahydrofuran- 3- ylamino)- pyrimidin-5- yl)phenyl)- acrylate | MS(ES): 483.2 (M + H) for C25H24ClFN4O3 1H NMR (300 MHz, DMSO-d₆) δ ppm 1.20-1.27 (m, 3 H) 1.53-1.74 (m, 2 H) 3.54-3.62 (m, 2 H) 3.72-3.82 (m, 2 H) 3.91-3.97 (m, 1 H) 4.48-4.58 (m, 2 H) 6.61 (d, J = 16.01 Hz,1 H) 7.30-7.41 (m, 1 H) 7.42-7.50 (m, 1 H) 7.58-7.68 (m, 2 H) 7.71-7.78 (m, 1 H) 8.01-8.07 (m, 1 H) 8.12-8.21 (m, 1 H) 8.42 (d, J = 3.77 Hz, 1 H) 8.49 (s, 1 H) 9.87-9.98 (m, 1 H) | 5-bromo-N2- (3-chloro-4- fluorophenyl)- N4- (tetrahydro- furan-3- yl)pyrimidine- 2,4-diamine Intermediate 381 And (E)-4-(3- ethoxy-3- oxoprop-1- enyl)phenyl- boronic acid |
| Example 940 | 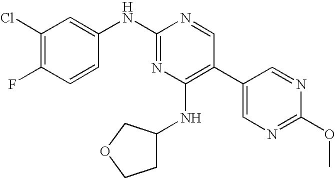 N2-(3-chloro-4- fluorophenyl)- 2′-methoxy-N4- (tetrahydrofuran- 3-yl)-5,5′- bipyrimidine- 2,4-diamine | MS(ES): 417.1 (M + H) for C19H18ClFN6O2 1H NMR (300 MHz, DMSO-d₆) δ ppm 1.84-1.98 (m, 1 H) 2.10-2.24 (m, 1 H) 3.52-3.62 (m, 1 H) 3.77 (s, 2 H) 3.89-3.97 (m, 4 H) 4.58 (s, 1 H) 6.91 (s, 1 H) 7.31 (t, J = 9.14 Hz, 1 H) 7.53-7.60 (m, 1 H) 7.79 (s, 1 H) 8.24 (dd, J = 6.97, 2.45 Hz, 1 H) 8.53 (s, 2 H) 9.47 (s, 1 H) | 5-bromo-N2- (3-chloro-4- fluorophenyl)- N4- (tetrahydro- furan-3- yl)pyrimidine- 2,4-diamine Intermediate 381 and 2- methoxy- pyrimidin-5- ylboronic acid |
| Example 941 | 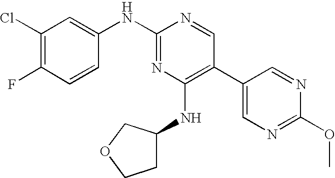 (S)-N2-(3- chloro-4- fluorophenyl)- 2′-methoxy-N4- (tetrahydrofuran- 3-yl)-5,5′- bipyrimidine- 2,4-diamine | MS(ES): 417.2 (M + H) for C19H18ClFN6O2 1H NMR (300 MHz, DMSO-d6) δ ppm 1.83-1.98 (m, 1 H) 2.12-2.25 (m, 1 H) 3.65-3.84 (m, 2 H) 3.90- 3.95 (m, 1 H) 3.96 (s, 3 H) 4.53-4.66 (m, 1 H) 6.93 (d, J = 6.22 Hz, 1 H) 7.31 (t, J = 9.14 Hz, 1 H) 7.58 (ddd, J = 6.83, 4.38, 1.98 Hz, 1 H) 7.80 (s, 1 H) 8.24 (dd, J = 6.97, 2.64 Hz, 1 H) 8.54 (s, 2 H) 9.47 (s, 1 H) | (S)-5-bromo-N2-(3-chloro- 4- fluorophenyl)- N4- (tetrahydro- furan-3- yl)pyrimidine- 2,4-diamine Intermediate 382 and 2- methoxy- pyrimidin-5- ylboronic acid |
| Example 942 | 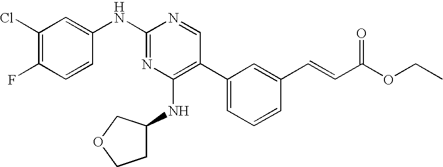 (S, E)-3-(3-(2- (3-chloro-4- fluorophenyl- amino)-4- (tetrahydrofuran- 3-ylamino)- pyrimidin-5- yl)phenyl)- acrylic acid | MS(ES): 455.0 (M + H) for C23H20ClFN4O3 1H NMR (300 MHz, DMSO-d6) δ ppm 1.87-2.01 (m, J = 12.48, 6.19, 6.19, 5.84 Hz, 1 H) 2.13-2.31 (m, 1 H) 3.66-3.86 (m, 3 H) 3.91-4.01 (m, 1 H) 4.57-4.69 (m, 1 H) 6.51-6.62 (m, 1 H) 7.27-7.36 (m, 1 H) 7.38- 7.52 (m, 2 H) 7.53-7.62 (m, 2 H) 7.63- 7.68 (m, 2H) 7.85 (s, 1 H) 8.22-8.29 (m, 1 H) 9.45 (s, 1 H) | (S)-5-bromo- N2-(3-chloro- 4- fluorophenyl)- N4- (tetrahydro- furan-3-yl)- pyrimidin- 2,4-diamine Intermediate 382 And (E)-4-(3- ethoxy-3- oxopro-1- enyl)phenyl- boronic acid |
| Example 943 | 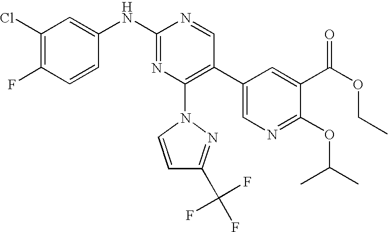 ethyl 5-(2-(3- chloro-4- fluorophenyl- amino)-4-(3- (trifluoromethyl)- 1H-pyrazol-1- yl)pyrimidin-5- yl)-2- isopropoxy- nicotinate | MS(ES): 565.0 (M + H) for C25H21ClF4N6O3 1H NMR (300 MHz, DMSO-d6) δ ppm 1.25 (t, J = 7.06 Hz, 1 H) 1.31 (d, J = 6.03 Hz, 5 H) 4.22 (q, J = 7.16 Hz, 2 H) 5.35 (spt, J = 6.19 Hz, 1 H) 7.04 (d, J = 2.64 Hz, 1 H) 7.40 (d, J = 9.04 Hz, 1 H) 7.75- 7.84 (m, 2 H) 8.07 (dd, J = 6.78, 2.64 Hz, 1 H) 8.21 (d, J = 2.45 Hz, 1 H) 8.51 (d, J = 1.70 Hz, 1 H) 8.80 (s, 1 H) 10.39 (s, 1 H) | Ethyl 2- isopropoxy-5- (4,4,5,5- tetramethyl- 1,3,2- dioxaborolan- 2- yl)nicotinate Intermediate 317 And Intermediate 115 |
| Example 944 | 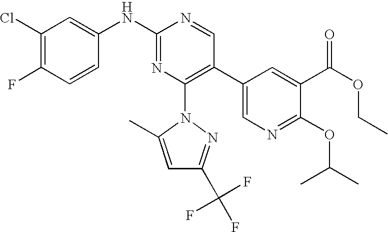 ethyl 5-(2-(3- chloro-4- fluorophenyl- amino)-4-(5- methyl-3- (trifluoromethyl)- 1H-pyrazol-1- yl)pyrimidin-5- yl)-2- isopropoxy- nicotinate | MS(ES): 579.0 (M + H) for C26H23ClF4N6O3 1H NMR (300 MHz, DMSO-d6) δ ppm 1.17-1.35 (m, 9 H) 2.36 (s, 3 H) 4.20 (q, J = 7.10 Hz, 2 H) 5.32 (spt, J = 6.15 Hz, 1 H) 6.77 (s, 1 H) 7.42 (t, J = 9.14 Hz, 1 H) 7.57 (d, J = 2.64 Hz, 1 H) 7.66 (ddd, J = 9.00, 4.19, 2.83 Hz, 1 H) 8.06 (dd, J = 6.78, 2.64 Hz, 1 H) 8.14 (d, J = 2.64 Hz, 1 H) 8.96 (s, 1 H) 10.42 (s, 1 H) | Ethyl 2- isopropoxy-5- (4,4,5,5- tetramethyl- 1,3,2- dioxaborolan- 2- yl)nicotinate Intermediate 317 And Intermediate 113 |
| Example 945 | 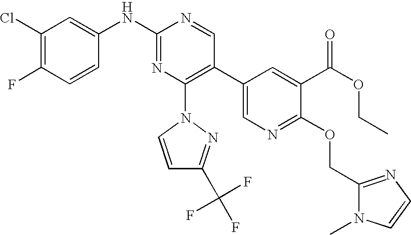 ethyl 5-(2-(3- chloro-4- fluorophenyl- amino)-4-(3- (trifluoromethyl)- 1H-pyrazol-1- yl)pyrimidin-5- yl)-2-((1- methyl-1H- imidazol-2- yl)methoxy)- nicotinate | MS(ES): 617.1 (M + H) for C27H21ClF4N8O3 1H NMR (300 MHz, DMSO-d6) δ ppm 1.16 (t, J = 7.06 Hz, 3 H) 3.73 (s, 3 H) 4.18 (q, J = 6.97 Hz, 2 H) 5.38-5.50 (m, 2 H) 6.86 (s, 1 H) 7.05 (d, J = 2.07 Hz, 1 H) 7.21 (s, 1 H) 7.42 (t, J = 9.04 Hz, 1 H) 7.72 (dd, J = 4.90, 4.14 Hz, 1 H) 7.84 (d, J = 1.88 Hz, 1 H) 8.08 (d, J = 8.85 Hz, 1 H) 8.35 (s, 1 H) 8.52 (br. s., 1 H) 8.82 (s, 1 H) 10.41 (s, 1 H) | ethyl 2-((1- methyl-1H- imidazol-2- yl)methoxy)- 5-(4,4,5,5- tetramethyl- 1,3-dioxolan- 2- yl)nicotinate Intermediate 320 And Intermediate 115 |
| Example 946 | 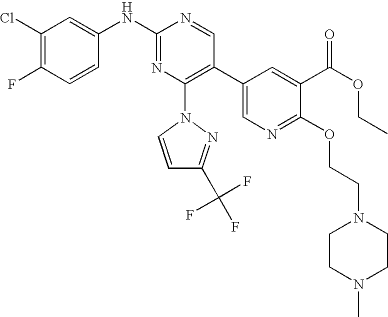 ethyl 5-(2-(3- chloro-4- fluorophenyl- amino)-4-(3- (trifluoromethyl)- 1H-pyrazol-1- yl)pyrimidin-5- yl)-2-(2-(4- methylpiperazin- 1- yl)ethoxy)- nicotinate | MS(ES): 649.1 (M + H) for C29H29ClF4N8O3 1H NMR (300 MHz, DMSO-d6) δ ppm 1.25 (t, J = 7.06 Hz, 3 H) 1.75 (s, 3 H) 2.13-2.29 (m, 4 H) 2.65-2.77 (m, 4 H) 3.35-3.42 (m, 2 H) 4.22 (q, J = 7.03 Hz, 2 H) 4.47 (t, J = 5.37 Hz, 2 H) 7.05 (d, J = 2.45 Hz, 1 H) 7.42 (t, J = 9.14 Hz, 1 H) 7.66-7.77 (m, 1H) 7.81 (d, J = 2.45 Hz, 1 H) 8.25 (d, J = 2.45 Hz, 1 H) 8.51 (d, J = 1.32 Hz, 1 H) 8.81 (s,1 H) 10.42 (s, 1 H) | ethyl 2-(2-(4- methylpiperazin- 1- yl)ethoxy)-5- (4,4,5,5- tetramethyl- 1,3,2- dioxaborolan- 2- yl)nicotinate Intermediate 316 And Intermediate 115 |
| Example 947 | 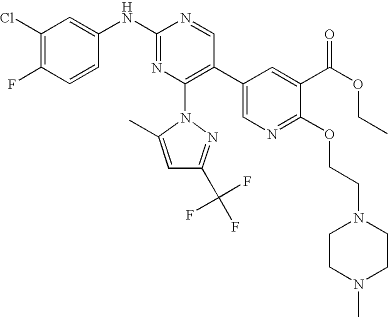 ethyl 5-(2-(3- choloro-4- fluorophenyl- amino)-4-(5- methyl-3- (trifluoromethyl)- 1H-pyrazol-1- yl)pyrimidin-5- yl)-2-(2-(4- methylpiperazin- 1- yl)ethoxy)- nicotinate | MS(ES): 663.1 (M + H) for C30H31ClF4N8O3 1H NMR (300 MHz, DMSO-d6) δ ppm 1.24 (t, J = 7.06 Hz, 4 H) 2.14 (s, 4 H) 2.22-2.40 (m, 8 H) 2.67 (t, J = 5.46 Hz, 2 H) 3.16 (d, J = 5.27 Hz, 1 H) 4.20 (q, J = 7.28 Hz, 2 H) 4.43 (t, J = 5.65 Hz, 2 H) 6.77 (s, 1 H) 7.42- (t, J = 9.14 Hz, 1 H) 7.56 (d, J = 2.45 Hz, 1 H) 7.65 (td, J = 6.45, 3.48 Hz, 1 H) 8.06 (dd, J = 6.59, 2.64 Hz, 1 H) 8.19 (d, J = 2.45 Hz, 1 H) 8.96 (s, 1 H) 10.44 (s, 1 H) | Ethyl 2-(2-(4- methylpiperazin- 1- yl)ethoxy)-5- (4,4,5,5- tetramethyl- 1,3,2- dioxaborolan- 2- yl)nicotinate Intermediate 316 And Intermediate 113 |
| Example 948 | 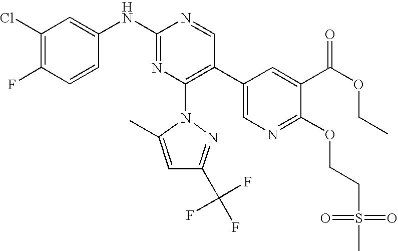 ethyl 5-(2-(3- chloro-4- fluorophenyl- amino)-4-(5- methyl-3- (trifluoromethyl)- 1H-pyrazol-1- yl)pyrimidin-5- yl)-2-(2- (methylsulfonyl)- ethoxy)- nicotinate | MS(ES): 643.0 (M + H) for C₂₆H₂₃ClF₄N₆O₅S 1H NMR (300 MHz, DMSO-d6) δ ppm 1.25 (t, J = 7.06 Hz, 3 H) 2.38 (s, 2 H) 3.03-3.10 (m, 4 H) 3.53-3.65 (m, 2 H) 4.21 (q, J = 7.16 Hz, 1 H) 4.35 (dt, J = 13.85, 6.83 Hz, 2 H) 6.82 (s, 1 H) 7.16 (d, J = 2.64 Hz, 1 H) 7.42 (t, J = 9.14 Hz,1 H) 7.58-7.69 (m, 1 H) 7.98-8.09 (m, 2 H) 8.28 (d, J = 2.45 Hz, 1 H) 8.88 (s, 1 H) 10.44 (s, 1H) | Ethyl 2-(2- (methylsul- fonyl)ethoxy)-5- (4,4,5,5- tetramethyl- 1,3,2- dioxaborolan- 2- yl)nicotinate Intermediate 321 And Intermediate 113 |
| Example 949 | 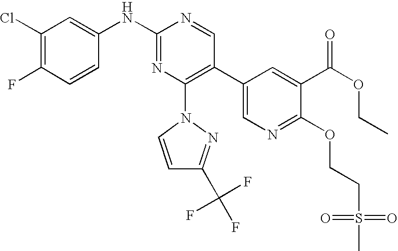 ethyl 5-(2-(3- chloro-4- fluorophenyl- amino)-4-(3- (tyrifluoromethyl)- 1H-pyrazol-1- yl)pyrimidin-5- yl)-2-(2- (methylsulfonyl)- ethoxy)- nicotinate | MS(ES): 629.0 (M + H) for C₂₅H₂₁ClF₄N₆O₅S 1H NMR (300 MHz, DMSO-d6) δ ppm 1.21-1.30 (m, 2 H) 3.03-3.11 (m, 8 H) 3.55-3.65 (m, 4 H) 4.13 (q, J = 6.59 Hz, 3 H) 4.33-4.42 (m, 3 H) 7.09 (d, J = 2.26 Hz, 1 H) 7.41 (t, J = 9.23 Hz, 1 H) 7.60 (d, J = 2.64 Hz, 1H) 7.65-7.73 (m, 1 H) 8.06 (dd, J = 6.78, 2.45 Hz, 1 H) 8.20 (d, J = 2.64 Hz, 1 H) 8.57 (s, 1 H) 8.71 (s, 1 H) 10.40 (s, 1 H) | Ethyl 2-(2- (methylsulfonyl)- ethoxy)-5- (4,4,5,5- tetramethyl- 1,3,2- dioxaborolan- 2- yl)nicotinate Intermediate 321 And Intermediate 115 |
| Example 950 | 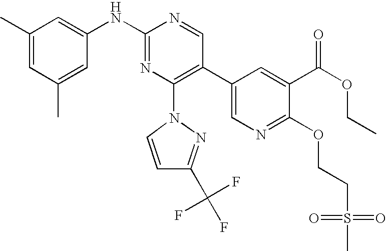 ethyl 5-(2-(3,5- dimethylphenyl amino)-4-(3- (trifluoromethyl)- 1H-pyrazol-1- yl)pyrimidin-5- yl)-2-(2- (methylsulfonyl)- ethoxy)- nicotinate | MS(ES): 605.2 (M + H) for C₂₇H₂₇F₃N₆O₅S 1H NMR (300 MHz, DMSO-d6) δ ppm 1.18 (t, J = 6.97 Hz, 3 H) 2.27 (s, 6 H) 3.07 (s, 3 H) 3.54-3.66 (m, 2 H) 4.14 (q, J = 7.10 Hz, 2 H) 4.38 (t, J = 7.16 Hz, 2 H) 6.69 (s, 1 H) 7.08 (d, J = 2.83 Hz, 1 H) 7.40 (s, 2 H) 7.60 (d, J = 2.64 Hz, 1 H) 8.21 (d, J = 2.45 Hz, 1 H) 8.54 (s, 1 H) 8.68 (s, 1 H) 10.07 (s, 1 H) | Ethyl 2-(2- (methylsulfonyl)- ethoxy)-5- (4,4,5,5- tetramethyl- 1,3,2- dioxaborolan- 2- yl)nicotinate Intermediate 321 And Intermediate 217 |
| Example 951 | 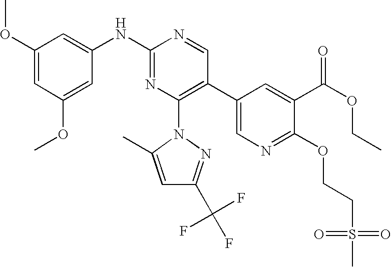 ethyl 5-(2-(3,5- dimethoxyphenyl- amino)-4-(5- methyl-3- (trifluoromethyl)- 1H-pyrazol-1- yl)pyrimidin-5- yl)-2-(2- (methylsulfonyl)- ethoxy)- nicotinate | MS(ES): 651.3 (M + H) for C28H29F3N6O7S 1H NMR (300 MHz, DMSO-d6) δ ppm 1.25 (t, J = 7.06 Hz,3 H) 2.36 (s,3 H) 3.06 (d, J = 5.27 Hz, 3 H) 3.57 (t, J = 6.97 Hz, 2 H) 3.72 (s, 6 H) 4.21 (q, J = 7.16 Hz, 2 H) 4.33 (t, J = 6.88 Hz, 2 H) 6.17- 6.24 (m, 1 H) 6.34 (t, J = 6.97 Hz, 1 H) 7.04 (d, J = 1.70 Hz, 2 H) 7.96-8.06 (m, 2 H) 8.85 (s, 1 H) 10.20 (s, 1 H) | Ethyl 2-(2- (methylsulfonyl)- ethoxy)-5- (4,4,5,5- tetramethyl- 1,3,2- dioxaborolan- 2- yl)nicotinate Intermediate 321 And Intermediate 216 |
| Example 952 | 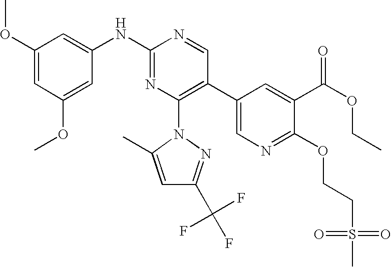 ethyl 5-(2-(3- chloro-4- fluorophenyl- amino)-4-(3- (trifluoromethyl)- 1H-pyrazol-1- yl)pyrimidin-5- yl)-2-(2- (methylsulfonyl)- ethoxy)- nicotinate | MS(ES): 629.1 (M + H) for C25H21F4N6O5S 1H NMR (300 MHz, DMSO-d6) δ ppm 1.21-1.30 (m, 3 H) 3.03-3.11 (m, 3 H) 3.55-3.65 (m, 2 H) 4.13 (q, J = 6.59 Hz, 2 H) 4.33-4.42 (m, 2 H) 7.09 (d, J = 2.26 Hz, 1 H) 7.41 (t, J = 9.23 Hz, 1 H) 7.60 (d, J = 2.64 Hz, 1H) 7.65-7.73 (m, 1 H) 8.06 (dd, J = 6.78, 2.45 Hz, 1 H) 8.20 (d, J = 2.64 Hz, 1 H) 8.57 (s, 1 H) 8.71 (s, 1 H) 10.40 (s,1 H) | ethyl 2-(2- (methylsulfonyl)- ethoxy)-5- (4,4,5,5- tetramethyl- 1,3,2- dioxaborolan- 2- yl)nicotinate Intermediate 321 And Intermediate 216 |
| Example 953 | 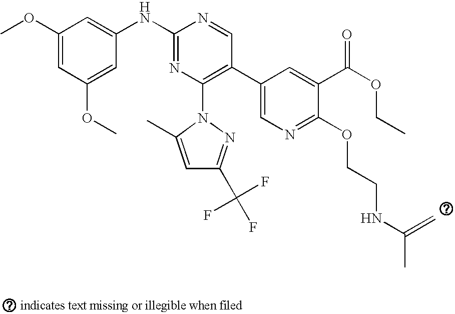 ethyl 2-(2- acetamidoethoxy)- 5-(2-(3,5- dimethoxy- phenylamino- 4-(5-methyl-3- (trifluoromethyl)- 1H-pyrazol-1- yl)pyrimidin-5- yl)nicotinate | MS(ES): 630.3 (M + H) for C29H30F3N7O6 80% purity, taken on to next step crude | ethyl 2-(2- acetamidoethoxy)- 5(4,4,5,5- tetramethyl- 1,3,2- dioxaborolan- 2- yl)nicotinate Intermediate 324 And Intermediate 216 |
| Example 954 | 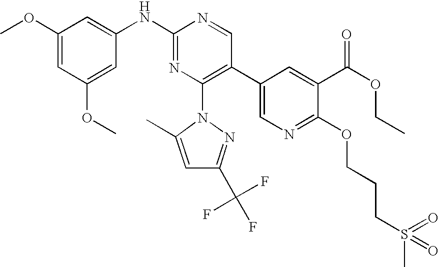 ethyl 5-(2-(3,5- dimethoxyphenyl- amino-4-(5- methyl-3- (trifluoromethyl)- 1H-pyrazol-1- yl)pyrimidin-5- yl)-2-(3- (methylsulfonyl)- propoxy)- nicotinate | MS(ES): 665.3 (M + H) for C29H31F3N6O7S 1H NMR (300 MHz, DMSO-d6) δ ppm 1.26 (t, J = 7.16 Hz, 3 H) 2.08-2.22 (m, 2 H) 2.32 (s, 3 H) 2.96-3.03(m, 3 H) 3.24-3.33 (m, 2 H) 3.72 (s, 6 H) 4.17- 4.26 (m, 2 H) 4.44 (t, J = 6.12 Hz, 2 H) 6.21 (t, J = 2.17 Hz, 1 H) 6.76 (s, 1 H) 7.05 (d, J = 2.26 Hz, 2 H) 7.59 (d, J = 2.64 Hz, 1 H) 8.24 (d, J = 2.45 Hz, 1 H) 8.94 (s,1 H) 10.20 (s, 1 H) | ethyl 2-(3- (methylsulfonyl)- propoxy)- 5-(4,4,5,5- tetramethyl- 1,3,2- dioxaborolan- 2- yl)nicotinate Intermediate 321-B And Intermediate 216 |
| Example 955 | 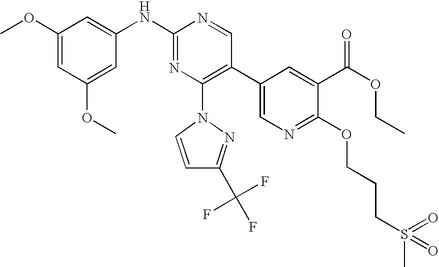 ethyl 5-(2-(3,5- dimethoxyphenyl- amino-4-(3- (trifluoromethyl)- 1H-pyrazol-1- yl)pyrimidin-5- yl)-2-(3- (methylsulfonyl)- propoxy)- nicotinate | MS(ES): 651.2 (M + H) for C28H29F3N6O7S 1H NMR (300 MHz, DMSO-d6) δ ppm 1.26 (t, J = 7.08 Hz, 3 H) 2.10-2.27 (m, 2 H) 2.97-3.04 (m, 3 H) 3.75 (s, 6 H) 3.90 (s, 2 H) 4.25 (q, J = 7.11 Hz, 2 H) 4.43-4.54 (m, 2 H) 6.21 (t, J = 2.27 Hz, 1 H) 7.03 (d, J = 2.46 Hz,1 H) 7.10 (d, J = 2.08 Hz, 2 H) 7.84 (d, J = 2.64 Hz, 1 H) 8.28 (d, J = 2.46 Hz, 1 H) 8.48 (dd, J = 2.64, 0.94 Hz, 1 H) 8.78 (s, 1 H) 10.15 (s, 1 H) | ethyl 2-(3- (methylsulfonyl)- propoxy)- 5-(4,4,5,5- tetramethyl- 1,3,2- dioxaborolan- 2- yl)nicotinate Intermediate 321-B And Intermediate 215 |

The compounds in the below table were prepared using the general method described above for

Example 214 using 1N sodium hydroxide and the starting material (SM) indicated.

| Ex | Compound | Data | SM |
|---|---|---|---|
| Example 956 | 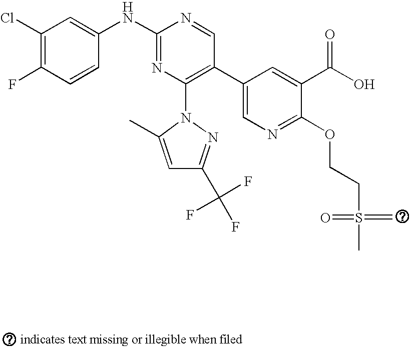 5-(2-(3-chloro- 4- fluorophenyl- amino)-4-(5- methyl-3- (trifluoromethyl)- 1H-pyrazol-1- yl)pyrimidin-5- yl)-2-(2- (methylsulfonyl)- ethoxy)- nicotinic acid | MS(ES): 615.2 (M + H) for C24H19ClF4N6O5S 1H NMR (300 MHz, DMSO-d6) δ ppm 2.44 (s, 5 H) 3.09 (s, 3 H) 3.67 (t, J = 6.78 Hz, 2 H) 4.48 (t, J = 6.50 Hz, 2 H) 6.82 (s, 1 H) 7.37-7.48 (m, 2 H) 7.59- 7.70 (m, 1 H) 8.06 (dd, J = 6.59, 2.26 Hz, 1 H) 8.30 (d, J = 2.07 Hz,1 H) 8.87 (s, 1 H) 10.45 (s, 1 H) | ethyl 5-(2-(3- chloro-4- fluorophenyl amino)-4-(5- methyl-3- (trifluoro- methyl)-1H- pyrazol-1- yl)pyrimidin- 5-yl)-2-(2- (methyl- sulfonyl)- ethoxy)- nicotinate Example 948 |
| Example 957 | 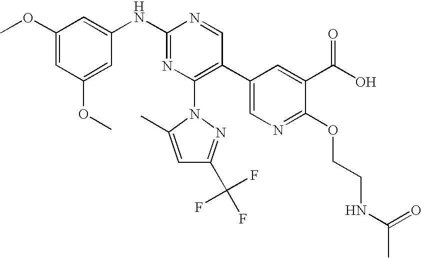 2-(2- acetamidoethoxy)- 5-(2-(3,5- dimthoxyphenyl- amino)-4-(5- methyl-3- (trifluoromethyl)- 1H-pyrazol-1- yl)pyrimidin-5- yl)nicotinic acid | MS(ES): 602.3 (M + H) for C27H26F3N7O6 1H NMR (300 MHz, DMSO-d6) δ ppm 1.78 (s, 3 H) 2.22 (s, 3 H) 3.72 (s, 7 H) 4.26 (t, J = 4.90 Hz, 2 H) 6.19 (s, 1 H) 6.71 (s, 1 H) 7.05 (s, 2 H) 7.51 (d, J = 1.70 Hz, 1 H) 7.57-7.68 (m, 1 H) 8.86 (s, 1 H) 10.16 (s, 1 H) | Ethyl 2-(2- acetamidoethoxy)- 5-(2- (3,5- dimethoxy- phenylamino)- 4-(5-methyl- 3- (trifluoro- methyl)-1H- pyrazol-1- yl)pyrimidin- 5- yl)nicotinate Example 953 |
| Example 958 | 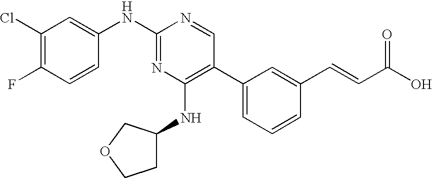 (E)-2-(3-(2-(3- chloro-4- fluorophenyl- amino)-4- (tetrahydrofuran- 3- ylamino)- pyrimidin-5- yl)phenyl)- acrylic acid | MS(ES): 455.0 (M + H) for C23H20ClF4N4O3 1H NMR (300 MHz, DMSO-d6) δ ppm 1.84-2.30 (m, 2 H) 3.52-4.00 (m, 4 H) 4.53-4.70 (m, 1 H) 6.49- 6.65 (m, 1 H) 7.25-7.37 (m, 1 H) 7.39- 7.53 (m, 3 H) 7.55-7.62 (m, 1 H) 7.64-7.72 (m, 2 H) 7.81-7.89 (m, 1 H) 8.20-8.31 (m, 1 H) 9.46 (s, 1 H) | (E)-ethyl-3- (3-(2-(3- chloro-4- fluorophenyl amino)-4- (tetrahydro- furan-3- ylamino)- pyrimidin-5- yl)phenyl)- acrylate Example 939 |
| Example 959 | 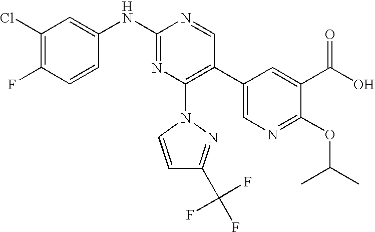 5-(2-(3-chloro- 4- fluorophenyl- amino)-4-(3- (trifluoromethyl)- 1H-pyrazol-1- yl)pyrimidin-5- yl)-2- isopropoxy- nicotinic acid | MS(ES): 537.0 (M + H) for C23H17ClF4N6O3 1H NMR (300 MHz, DMSO-d6) δ ppm 1.30 (d, J = 6.03 Hz, 6 H) 5.20- 5.42 (m, 1 H) 7.03 (d, J = 2.45 Hz, 1 H) 7.42 (t, J = 9.14 Hz, 1 H) 7.66-7.79 (m, 2 H) 8.08 (dd, J = 6.69, 2.35 Hz, 2 H) 8.47 (br. s., 1 H) 8.78 (s, 1 H) 10.40 (s, 1H) | Ethyl 5-(2-(3- chloro-4- fluorophenyl amino)-4-(3- (trifluoro- methyl)-1H- pyrazol-1- yl)pyrimidin- 5-yl)-2- isopropoxy- nicotinate Example 943 |
| Example 960 | 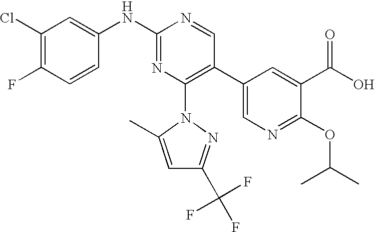 5-(2-(3-chloro- 4- fluorophenyl- amino)-4-(5- methyl-3- (trifluoromethyl)- 1H-pyrazol-1- yl)pyrimidin-5- yl)-2- isopropoxy- nicotinic acid | MS(ES): 551.0 (M + H) for C24H19ClF4N6O3 1H NMR (300 MHz, DMSO-d6) δ ppm 1.28 (d, J = 6.22 Hz, 6 H) 2.35 (s, 3 H) 5.31 (quin, J = 6.08 Hz, 1 H) 6.75 (s, 1 H) 7.42 (t, J = 9.14 Hz, 1 H) 7.59- 7.70 (m, 2 H) 7.98-8.10 (m, 2 H) 8.94 (s, 1 H) 10.41 (s, 1 H) 12.82 (s,1H) | Ethyl 5-(2-(3- chloro-4- fluorophenyl amino)-4-(5- methyl-3- (trifluoro- methyl)-1H- pyrazol-1- yl)pyrimidin- 5-yl)-2- isopropoxy- nicotinate Example 944 |
| Example 961 | 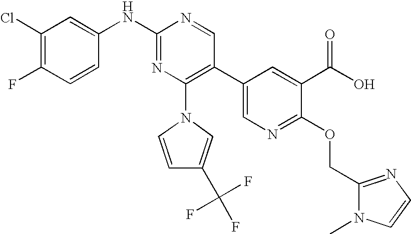 5-(2-(3-chloro- 4- fluorophenyl- amino)-4-(3- (trifluoromethyl)- 1H-pyrazol-1- yl)pyrimidin-5- yl)-2-((1- methyl-1H- imidazol-2- yl)methoxy)- nicotinic acid | MS(ES): 589.0 (M + H) for C25H17ClF4N8O3 1H NMR (300 MHz, DMSO-d6) δ ppm 3.94 (s, 4 H) 5.71 (s, 2 H) 7.05 (d, J = 2.83 Hz, 1 H) 7.43 (t, J = 9.14 Hz, 1 H) 7.59-7.65 (m, 1 H) 7.65-7.77 (m, 2 H) 7.96 (d, J = 2.45 Hz, 1 H) 8.08 (dd, J = 6.50, 2.54 Hz, 1 H) 8.27 (d, J = 2.45 Hz, 1 H) 8.54 (d, J = 1.51 Hz, 1 H) 8.79 (s, 1 H) 10.43 (s, 1 H) | Ethyl 5-(2-(3- choloro-4- fluorophenyl amino)-4-(3- (trifluoromethyl)- 1H- pyrazol-1- yl)pyrimidin- 5-yl)-2-((1- methyl-1H- imidazol-2- yl)methoxy)- nicotinate Example 945 |
| Example 962 | 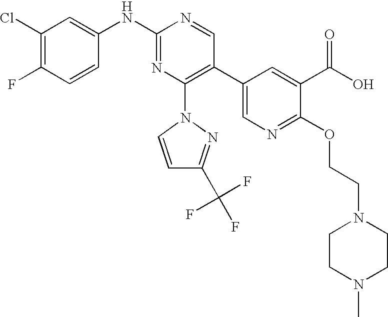 5-(2-(3-chloro- 4- fluorophenyl- amino)-4-(3- (trifluoromethyl)- 1H-pyrazol-1- yl)pyrimidin-5- yl)-2-(2-(4- methylpiperazin- 1- yl)ethoxy)- nicotinic acid | MS(ES): 621.0 (M + H) for C27H25ClF4N8O3 1H NMR (300 MHz, DMSO-d6) δ ppm 2.83 (br. s., 3 H) 3.38 (s, 2 H) 3.40- 3.53 (m, 2 H) 3.63 (d, J = 5.65 Hz, 5 H) 4.77 (br. s., 3 H) 7.05 (d, J = 2.45 Hz, 1 H) 7.42 (t, J = 9.14 Hz, 2 H) 7.67- 7.77 (m, 1 H) 7.90 (d, J = 2.45 Hz, 1 H) 8.09 (dd, J = 6.50, 2.35 Hz, 1 H) 8.30 (d, J = 2.26 Hz, 1 H) 8.51 (s, 1 H) 8.80 (s, 1 H) 10.44 (s, 1 H) | Ethyl 5-(2-(3- chloro-4- fluorophenyl amino)-4-(3- (trifluoro- methyl)-1H- pyrazol-1- yl)pyrimidin- 5-yl)-2-(2-(4- methyl- piperazin-1- yl)ethoxy)- nicotinate Example 946 |
| Example 963 | 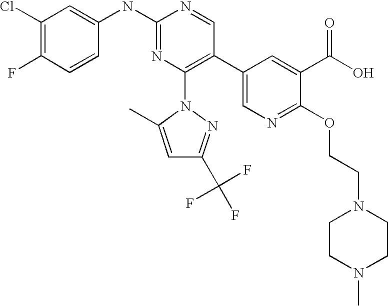 5-(2-(3-chloro- 4- fluorophenyl- amino)-4-(5- methyl-3- (trifluoromethyl)- 1H-pyrazol-1- yl)pyrimidin-5- yl)-2-(2-(4- methylpiperazin- 1- yl)ethoxy)- nicotinic acid | MS(ES): 635.0 (M + H) for C28H27ClF4N8O3 1H NMR (300 MHz, DMSO-d6) δ ppm 2.37 (s, 3 H) 2.48-2.52 (s, 8H) 2.70-2.80 (m, 5 H) 4.41-4.53 (m, 2 H) 6.78 (s, 1 H)7.43 (t, J = 9.04 Hz, 1 H) 7.66 (d, J = 2.64 Hz, 2 H) 8.08 (dd, J = 7.60, 2.35 Hz, 1 H) 8.15 (d, J = 2.45 Hz, 1 H) 8.95 (s, 1 H) 10.45 (s, 1 H) | Ethyl 5-(2-(3- chloro-4- fluorophenyl amino)-4-(5- methyl-3- (trifluoro- methyl)-1H- pyrazol-1- yl)pyrimidin- 5-yl)-2-(2-(4- methyl- piperazin-1- yl)ethoxy)- nicotinate Example 947 |
| Example 964 | 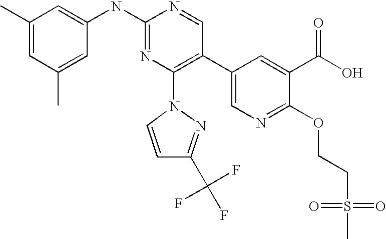 5-(2-(3,5- dimethylphenyl amino)-4-(3- (trifluoromethyl)- 1H-pyrazol-1- yl)pyrimidin-5- yl)-2-(2- (methylsulfonyl)- sulfonyl)- nicotinic acid | MS(ES): 577.2 (M + H) for C25H23F3N6O5S 1H NMR (300 MHz, DMSO-d6) δ ppm 2.29 (s, 5 H) 3.10 (s, 4 H) 3.71 (t, J = 6.69 Hz, 3 H) 4.49-4.61 (m, 1H) 6.70 (s, 1 H) 7.08 (d, 2 H) 7.40 (s, 2 H) 8.05 (d, 1 H) 8.44 (s, 1 H) 8.58 (d, 1 H) 8.67 (s, 1 H) 10.12 (s, 1 H) | Ethyl 5-(2- (3,5- dimethylphenyl- ylamino)-4- (3- (trifluoromethyl)- 1H- pyrazol-1- yl)pyrimidin- 5-yl)-2-(2- (methylsulfonyl)- ethoxy)- nicotinate Example 950 |
| Example 965 | 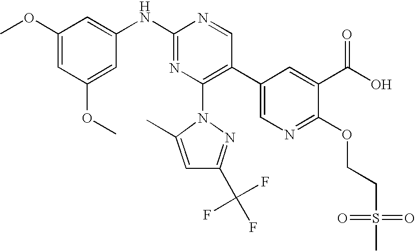 5-(2-(3,5- dimethoxy- phenylamino)-4-(5- methyl-3- (trifluoromethyl)- 1H-pyrazol-1- yl)pyrimidin-5- yl)-2-(2- (methylsulfonyl)- ethoxy)- nicotinic acid | MS(ES): 623.2 (M + H) for C26H25F3N6O7S 1H NMR (300 MHz, DMSO-d6) δ ppm 2.42 (s, 4 H) 3.09 (s, 3 H) 3.67 (t, J = 6.69 Hz, 2 H) 3.73 (s, 6 H) 4.48 (t, J = 6.31 Hz, 2 H) 6.22 (t, J = 1.98 Hz, 1 H) 6.80 (s, 1 H) 7.04 (d, J = 1.88 Hz, 2 H) 7.46 (d, 1H) 8.33 (d, 1 H) 8.85 (s, 1 H) 10.21 (s, 1 H) | Ethyl 5-(2- (3,5- dimethoxy- phenylamino)- 4-(5-methyl- 3- (trifluoro- methyl)-1H- pyrazol-1- yl)pyrimidin- 5-yl)-2-(2- (methyl- sulfonyl)- ethoxy) nicotinate Example 951 |
| Example 966 | 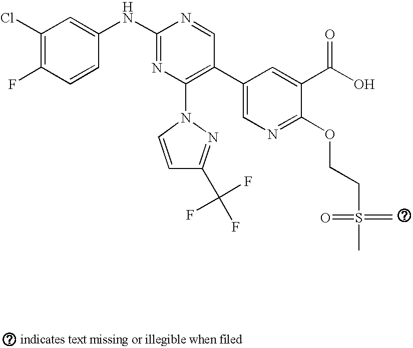 5-(2-(3-chloro- 4- fluorophenyl- amino)-4-(3- (trifluoromethyl)- 1H-pyrazol-1- yl)pyrimidin-5- yl)-2-(2- (methylsulfonyl)- ethoxy)- nicotinic acid | MS(ES): 601.1 (M + H) for C23H17ClF4N6O5S 1H NMR (300 MHz, DMSO-d6) δ ppm 3.09 (s, 3 H) 3.70 (t, J = 6.78 Hz, 2 H) 4.53 (t, J = 6.59 Hz, 2 H) 7.10 (d, J = 2.64 Hz, 1 H) 7.43 (t, J = 9.14 Hz, 1 H) 7.67-7.76 (m, 1 H) 7.97 (s, 1 H) 8.07 (dd, J = 6.59, 2.26 Hz, 1 H) 8.38 (br. s., 1 H) 8.59 (s, 1 H) 8.72 (s, 1 H) 10.44 (s, 1 H) | Ethyl 5-(2-(3- chloro-4- fluophenyl amino)-4-(3- (trifluoromethyl)- 1H- pyrazol-1- yl)pyrimidin- 5-yl)-2-(2- (methyl- sulfonyl)- ethoxy)- nicotinic acid Example 952 |
| Example 967 | 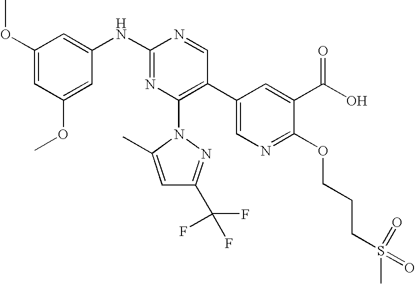 5-(2-(3,5- dimethoxy- phenylamino)-4-(5- methyl-3- (trifluoromethyl)- 1H-pyrazol-1- yl)pyrimidin-5- yl)-2-(3- (methylsulfonyl)- propoxy)- nicotinic acid | MS(ES): 637.2 (M + H) for C27H27F3N6O7S 1H NMR (300 MHz, DMSO-d6) δ ppm 2.09-2.20 (m, 2 H) 2.31 (s, 3 H) 2.99 (s, 3 H) 3.25 (br. s., 1 H) 3.72(s, 6 H) 4.42 (t, J = 6.22 Hz, 2 H) 6.21 (t, J = 2.17 Hz, 1 ) 6.74 (s, 1 H) 7.05 (d, J = 2.26 Hz, 2 H) 7.64 (d, J = 2.45 Hz, 1 H) 8.11 (br. s., 1 H) 8.92 (s, 1 H) 10.18 (s, 1 H) 12.96 (br. s., 1 H) | ethyl 5-(2- (3,5- dimethoxy- phenylamino)- 4-(5-methyl- 3- (trifluoro- methyl)-1H- pyrazol-1- yl)pyrimidin- 5-yl)-2-(3- (methyl- sulfonyl)- propoxy) nicotinate Example 954 |
| Example 968 | 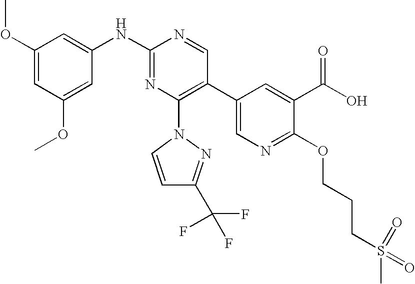 5-(2-(3,5- dimethoxy- phenylamino)-4-(3- (trifluoromethyl)- 1H-pyrazol-1- yl)pyrimidin-5- yl)-2-(3- (mehtylsulfonyl)- propoxy)- nicotinic acid | MS(ES): 623.1 (M + H) for C26H25F3N6O7S 1H NMR (300 MHz, DMSO-d6) δ ppm 2.01-2.15 (m, 2 H) 2.97 (s, 3 H) 3.25-3.34 (m, 20 H) 3.74 (s, 6 H) 4.32 (t, UJ = 6.23 Hz, 2 H) 6.18 (t, J = 2.17 Hz, 1 H) 6.95 (d, J = 2.64 Hz, 1 H) 7.13 (d, J = 2.08 Hz, 2 H) 7.40 (d, J = 2.08 Hz, 1 H) 7.70 (d, J = 2.27 Hz, 1 H) 8.22 (d, J = 1.51 Hz, 1 H) 8.69 (s, 1 H) 10.10 (s, 1 H) | Ethyl 5-(2- (3,5- dimethoxyphenyl- amino)- 4-(3- (trifluoromethyl)- 1H- pyrazol-1- yl)pyrimidin- 5-yl)-2-(3- (methyl- sulfonyl)- propoxy) nicotinate Example 955 |

The compounds in the below table were prepared using the general method described above for Example 824 and the starting material (SM) indicated.

| Ex | Compound | Data | SM |
|---|---|---|---|
| Example 969 | 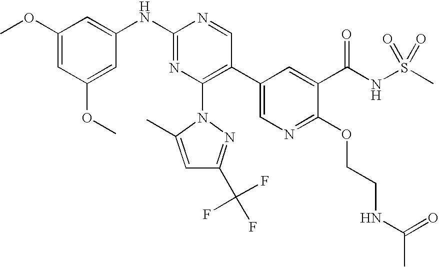 2-(2- acetamidoethoxy)-5- (2-(3,5- dimethoxyphenyl- amino)-4-(5-methyl-3- (trifluoromethyl)-1H- pyrazol-1- yl)pyrimidin-5-yl)-N- (methylsulfonyl)- nicotinamide | MS(ES): 679.3 (M + H) for C28H29F3N8O7S 1H NMR (300 MHz, DMSO-d6) δ ppm 1.81 (s, 2 H) 2.33 (s, 2 H) 3.33 (br. s., 5 H) 3.38-3.46 (m, 2 H) 3.67-3.75 (m, 6 H) 4.37 (t, J = 5.75 Hz, 2 H) 6.22 (t, J = 2.17 Hz, 1 H) 6.77 (s, 1 H) 7.05 (d, J = 2.07 Hz, 2 H) 7.71 (d, J = 2.45 Hz, 1 H) 7.99-8.03 (m, 1 H) 8.94- (s, 1 H) 10.21 (s, 1 H) 11.50 (s, 1 H) | 2-(2- acetamido- ethoxy)-5-(2-(3,5- dimethoxy- phenylamino)-4- (5-methyl-3- (trifluoro- methyl)-1H- pyrazol-1- yl)pyrimidin- 5-yl)nicotinic acid Example 957 |
| Example 970 | 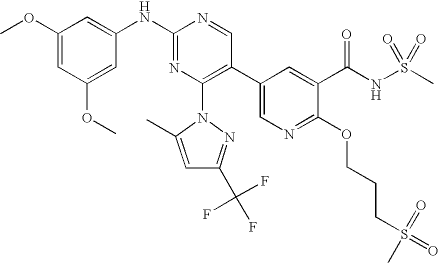 5-(2-(3,5- dimethoxyphenyl- amino)-4-(5-methyl-3- (trifluoromethyl)-1H- pyrazol-1- yl)pyrimidin-5-yl)-N- (methylsulfonyl)-2- (3- (methylsulfonyl)- propoxy) nicotinamide | MS(ES): 714.3 (M + H) for C28H30F3N7O8S2 1H NMR (300 MHz, DMSO-d6) δ ppm 2.11-2.23 (m, 2 H) 2.32 (s, 3 H) 2.99 (s, 3 H) 3.27 (br. s., 2 H) 3.33 (d, J = 3.77 Hz, 3 H) 3.72 (s, 6 H) 4.41 (t, J = 6.22 Hz, 2 H) 6.21 (t, J = 2.07 Hz, 1 H) 6.77 (s, 1 H) 7.05 (d, J = 2.07 Hz, 2 H) 7.68 (d, J = 2.26 Hz, 1 H) 7.99 (d, J = 2.45 Hz, 1 H) 8.94 (s, 1 H) 10.21 (s, 1 H) 11.80 (br. s., 1 H) | 5-(2-(3,5- dimethoxy- phenylamino)-4- (5-methyl-3- (trifluoro- methyl)-1H- pyrazol-1- yl)pyrimidin- 5-yl)-2-(3- (methyl- sulfonyl)- propoxy) nicotinic acid Example 967 |
| Example 971 | 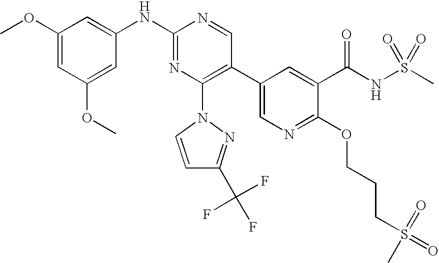 (2-(3,5- dimethoxyphenyl- amino)-4-(3- (trifluoromethyl)-1H- pyrazol-1- yl)pyrimidin-5-yl)-N- (methylsulfonyl)-2- (3- (methylsulfonyl)- propoxy)nicotinamide | MS(ES): 700.2 (M + H) for C27H28F3N7O8S2 1H NMR (300 MHz, DMSO-d6) δ ppm 2.15-2.24 (m, 2 H) 3.00 (s, 3 H) 3.25-3.37 (m, 5 H) 3.75 (s, 6 H) 4.46 (t, J = 6.33 Hz, 2 H) 6.22 (t, J = 2.17 Hz, 1 H) 7.04 (d, J = 2.45 Hz, 1 H) 7.11 (d, J = 2.27 Hz, 2H) 7.86 (d, J = 2.45 Hz, 1 H) 8.17 (d, J = 2.45 Hz, 1 H) 8.45 (d, J = 1.70 Hz, 1 H) 8.77 (s, 1 H) 10.17 (s, 1 H) | 5-(2-(3,5- dimethoxy- phenylamino)-4- (3- trifluoro- methyl)-1H- pyrazol-1- yl)pyrimidin- 5-yl)-2-(3- (methyl- sulfonyl)- propoxy)- nicotinic acid Example 968 |
| Example 972 | 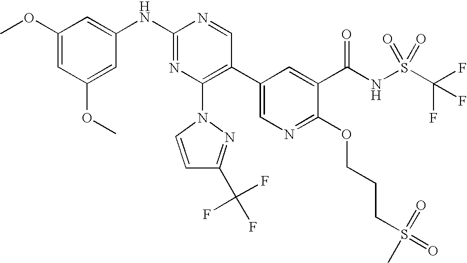 5-(2-(3,5- dimethoxyphenyl- amino)-4-(3- (trifluoromethyl)-1H- pyrazol-1- yl)pyrimidin-5-yl)-2- (3- (methylsulfonyl)- propoxy)-N- (trifluoromethyl- sulfonyl)nicotinamide | MS(ES): 754.2 (M + H) for C₂₇H₂₅F₆N₇O₈S₂ 1H NMR (300 MHz, DMSO-d6) δ ppm 2.03-2.19 (m, 2 H) 2.97 (s, 3 H) 3.36 (s, 2 H) 3.74 (s, 6 H) 3.83 (s, 1H) 4.38 (t, J = 6.14 Hz, 2 H) 6.19 (t, 1 H) 6.95 (d.J = 2.64 Hz, 1 H) 7.12 (d, J = 2.27 Hz, 2 H) 7.63 (d, J = 2.45 Hz, 1 H) 7.99 (d, J = 2.46 Hz, 1 H) 8.34 (d, J = 0.94 Hz, 1 H) 8.73 (s, 1 H) 10.12 (s, 1 H) | 5-(2-(3,5- dimethoxy- phenylamino)-4- (3- (trifluoro- methyl)-1H- pyrazol-1- yl)pyrimidin- 5-yl)-2-(3- (methyl- sulfonyl)- propoxy)- nicotinic acid Example 968 |

### Example 973

### (E)-5-(2-(3,5-dimethoxyphenylamino)-4-(5-methyl-3-(trifluoromethyl)-1H-pyrazol-1-yl)pyrimidin-5-yl)-N'-hydroxy-2-methoxynicotinimidamide

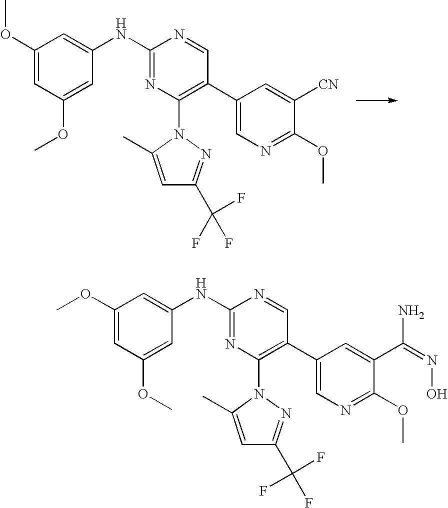

5-(2-(3,5-dimethoxyphenylamino)-4-(5-methyl-3-(trifluoromethyl)-1H-pyrazol-1-yl)pyrimidin-5-yl)-2-methoxynicotinonitrile Example 868 (0.10 g, 0.20 mmol), and hydroxylamine (0.018 ml, 0.30 mmol) were suspended in ethanol (4.6 mL) to give a white suspension. The mixture was heated at 80° C. for 2 hours then concentrated in vacuo. The residue was triturated with acetonitrile and dried under high vacuum to obtain the title compound as an off-white solid (0.095 g).

MS(ES): 545.2 (M+H) for C₂₄H₂₃F₃N₈O₄

¹H-NMR (400 MHz, DMSO-d6): δ ppm 2.27 (s, 3H) 3.72 (s, 7 H) 3.87 (s, 3H), 5.69 (s, 2H) 6.21 (s, 1H) 6.73 (s, 1H) 7.04 (d, J=1.88 Hz, 2H) 7.57 (d, J=2.26 Hz, 1H) 7.81 (d, J=2.45 Hz, 1H) 8.88 (s, 1H) 9.58 (s, 1H)10.19 (s, 1H)

### Example 974

### 3-(5-(2-(3,5-dimethoxyphenylamino)-4-(5-methyl-3-(trifluoromethyl)-1H-pyrazol-1-yl)pyrimidin-5-yl)-2-methoxypyridin-3-yl)-1,2,4-oxadiazol-5(4H)-thione

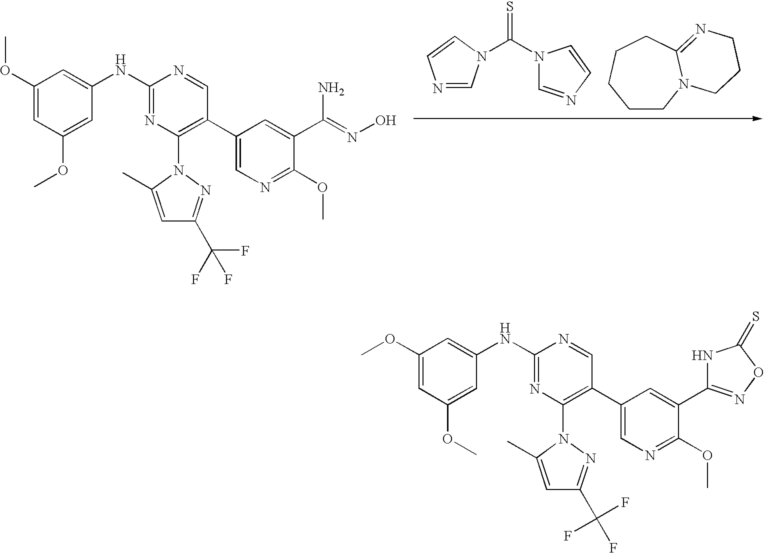

(E)-5-(2-(3,5-dimethoxyphenylamino)-4-(5-methyl-3-(trifluoromethyl)-1H-pyrazol-1-yl)pyrimidin-5-yl)-N'-hydroxy-2-methoxynicotinimidamide Example 973 (0.20 g, 0.37 mmol), di(1H-imidazol-1-yl)methanethione (0.099 g, 0.55 mmol) and DBU (0.222 ml, 1.47 mmol) were suspended in acetonitrile (8.16 mL). The mixture was stirred at room temperature for 1.5 hours then concentrated in vacuo. The residue was purified by flash chromatography: 4 g silica column, 3-30% methanol in chloroform over 25 min. The relevant fractions were pooled and resulting material was dried and purified by reverse phase HPLC: 65-95% methanol in 0.1% formic acid-water (pH 3) using a 19mm×100 mm 5 μm waters T3 C18 column. Evaporation of fractions gave the title compound as an off-white solid (0.05 g).

MS(ES): 587.2 (M+H) for C₂₅H₂₁F₃N₈O₄S

¹H-NMR (300 MHz, DMSO-d6): δ ppm 2.37 (s, 3H) 3.72 (s, 7 H) 3.95 (s, 3H) 6.22 (t, J=2.07 Hz, 1H) 6.77 (s, 1H) 7.05 (d, J=2.07 Hz, 2H) 7.82 (d, J=2.45 Hz, 1H) 8.09 (d, J=1.88 Hz, 1H) 8.93 (s, 1H) 10.22 (s, 1H)

### Example 975

### 3-(5-(2-(3,5-dimethoxyphenylamino)-4-(5-methyl-3-(trifluoromethyl)-1H-pyrazol-1-yl)pyrimidin-5-yl)-2-methoxypyridin-3-yl)-1,2,4-oxadiazol-5(4H)-one

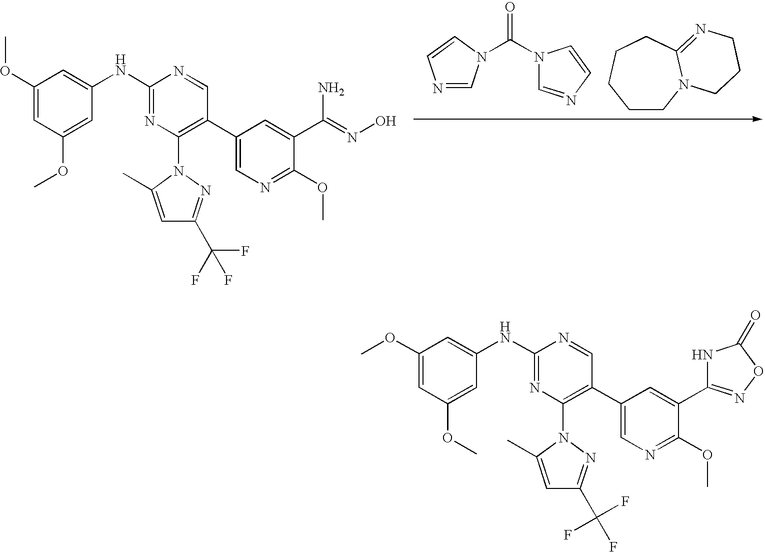

(E)-5-(2-(3,5-dimethoxyphenylamino)-4-(5-methyl-3-(trifluoromethyl)-1H-pyrazol-1-yl)pyrimidin-5-yl)-N′-hydroxy-2-methoxynicotinimidamide Example 973 (0.0951 g, 0.17 mmol), CDI (0.042 g, 0.26 mmol) and DBU (0.053 ml, 0.35 mmol) were suspended in 1,4-dioxane (3.5 mL). The mixture was stirred at room temperature overnight then concentrated in vacuo. The residue was purified by reverse phase flash chromatography: 50 g C18 column, 5-75% acetonitrile in water over 25 min. Relevant fractions pooled and evaporated to give the title compound as a white solid (0.029g).

MS(ES): 571.2 (M+H) for C₂₅H₂₁F₃N₈O₅

¹H-NMR (300 MHz, DMSO-d6): δ ppm 2.38 (s, 4H) 3.72 (s, 6 H) 3.95 (s, 3H) 6.22 (br, s., 1H) 6.77 (s, 1H) 7.04 (s, 2H) 7.72 (s, 1H) 8.15 (s, 1H) 8.93 (s, 1H) 10.22 (s, 1H)

The following compounds were prepared using the general method described for Example 1 using tris(dibenzyledeneacetone)-dipalladium(0), 2-dicyclohexyl phosphino-2′,4′,6′-triiso-propyl-1,1′-biphenyl, sodium carbonate and the starting materials (SM) indicated.

| Ex | Compound | Data | SM |
|---|---|---|---|
| Example 976 | 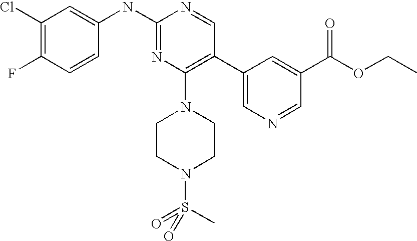 Ethyl 5-(2-(3-chloro-4- fluorophenylamino)-4-(4- (methylsulfonyl)piperazin- 1-yl)pyrimidin-5- yl)nicotinate | MS(ES): 535 (M + 1) for C₂₃H₂₄ClFN₆O₄S ¹H NMR (300 MHz, DMSO-D6) δ ppmn 1.34 (t, J = 7.06 Hz, 3 H) 2.87 (s, 3 H) 3.02-3.14 (m, 4 H) 3.24-3.48 (m, 4 H) 4.37 (q, J = 7.10 Hz, 2 H) 7.36 (t, J = 9.14 Hz, 1 H) 7.56-7.75 (m, 1 H) 8.10 (dd, J = 6.78, 2.64 Hz, 1 H) 8.20(s, 1 H) 8.37 (t, J = 2.07 Hz, 1 H) 8.93 (d, J = 2.07 Hz, 1 H) 9.03 (d, J = 1.88 Hz, 1 H) 9.83 (s, 1 H) | 5-bromo-N-(3- chloro-4- fluorophenyl)-4- (4- (methylsulfonyl)- piperazin-1- yl)pyrimidin-2- amine (Intermediate 130) and 3- (ethoxycarbonyl) pyridine-5- boronic acid pinacol ester |
| Example 977 | 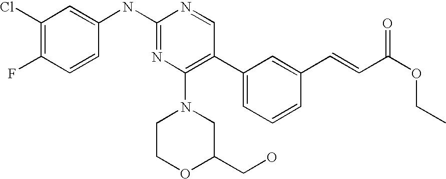 (E)-ethyl 3-(3-(2-(3-chloro- 4-fluorophenylamino)-4- (2- (hydroxymethyl)morpholino)- pyrimidin-5- yl)phenyl)acrylate | MS(ES): 514 (M + 1) for C₂₆H₂₆ClFN₄O₄ ¹H NMR (300 MHz, DMSO-D6) δ ppm 1.27 (t, J = 7.06 Hz, 3 H) 2.63- 3.02 (m, 2 H) 3.13-3.63 (m, 5 H) 3.63-3.99 (m, 2 H) 4.20 (q, J = 7.16 Hz, 2 H) 6.74 (d, J = 16.01 Hz, 1 H) 7.36 (t, J = 9.14 Hz, 1 H) 7.43-7.58 (m, 2H) 7.58-7.79 (m, 3 H) 7.76- 7.91 (m, 1 H) 7.98- 8.22 (m, 2 H) 9.83 (s, 1 H) | (4-(5-bromo-2-(3- chloro-4- fluorophenylamino)- pyrimidin-4- yl)morpholin-2- yl)methanol Intermediate 383 and Ethyl 3- borocinamate |
| Example 978 | 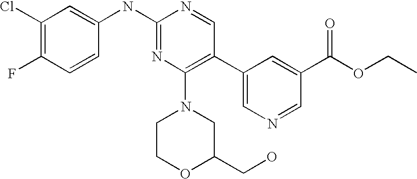 Ethyl 5-(2-(3-chloro-4- fluorophenylamino)-4-(2- (hydroxymethyl)morpholino)- pyrimidin-5-yl)nicotinate | MS(ES): 488 (M + 1) for C₂₃H₂₃ClFN₅O₄. ¹H NMR (300 MHz, DMSO-D6) δ ppm 1.34 (t, J = 7.06 Hz, 3 H) 2.60- 2.80 (m, 1 H) 2.79-2.99 (m, 1 H) 3.06-3.58 (m, 5 H) 3.75 (d, J = 14.69 Hz, 2 H) 4.37 (q, J = 6.97 Hz, 2 H) 7.35 (t, J = 9.14 Hz, 1 H) 7.52-7.73 (m, 1 H) 7.97-8.14 (m,1 H) 8.17 (s, 1 H) 8.37 (t, J = 2.07 Hz, 1 H) 8.91 (d, J = 2.26 Hz, 1 H) 9.02 (d, J = 1.88 Hz, 1 H) 9.88 (s, 1 H) | (4-(5-bromo-2-(3- chloro-4- fluorophenyl- amino)pyrimidin-4- yl)morpholin-2- yl)methanol Intermediate 383 and 3- (ethoxycarbonyl) pyridine-5- boronic acid pinacol ester |
| Example 979 | 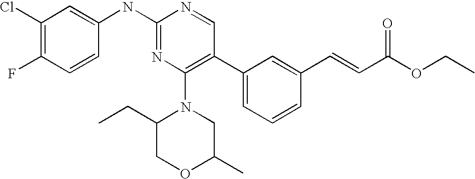 (E)-ethyl 3-(3-(2-(3-chloro- 4-fluorophenylamino)-4- (5-ethyl-2- methylmorpholino)- pyrimidin-5-yl)- phenyl)acrylate | MS(ES): 525 (M + 1) for C₂₈H₃₀ClFN₄O₃ ¹H NMR (300 MHz, DMSO-D6) δ ppm 0.39- 0.73 (m, 3 H) 0.98 (d, J = 5.65 Hz, 3 H) 1.25 (t, J = 7.16 Hz, 3 H) 1.39- 1.79 (m, 2 H) 2.61-2.90 (m, 1 H) 3.39-3.71 (m, 5 H) 4.19 (q, J = 7.03 Hz, 2 H) 6.72 (d, J = 16.01 Hz, 1 H) 7.38 (t, J = 9.14 Hz, 1 H) 7.42-7.60 (m, 3 H) 7.58-7.90 (m, 3 H) 7.98 (d, J = 0.94 Hz, 1 H) 8.02- 8.18 (m, 1 H) 9.88 (s, 1 H) | 5-bromo-N-(3- chloro-4- fluorophenyl)-4- (5-ethyl-2- methylmorpholino)- pyrimidin-2- amine Intermediate 384 and Ethyl 3- boronocinamate |
| Example 980 | 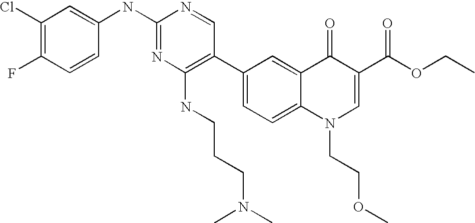 Ethyl 6-(2-(3-chloro-4- fluorophenylamino)-4-(3- (dimethylamino)propyl- amino)pyrimidin-5-yl)-1-(2- methoxyethyl)-4-oxo-1,4- dihydroquinoline-3- carboxylate | MS(ES): 597 (M + 1) for C₃₀H₃₄ClFN₆O₄ | 5-Bromo-N²-(3- chloro-4-fluoro- phenyl)-N⁴-(3- dimethylamino- propyl)- pyrimidin-2,4- diamine Intermediate 26 and ethyl 1-(2- methoxyethyl)-4- oxo-6-(4,4,5,5- tetramethyl-1,3,2- dioxaborolan-2- yl)-1,4- dihydroquinoline- 3-carboxylate Intermediate 134 |
| Example 981 | 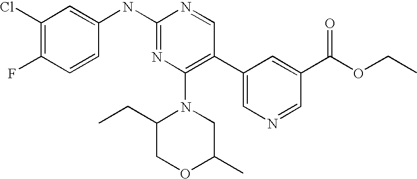 Ethyl 5-(2-(3-chloro-4- fluorophenylamino)-4-(5- ethyl-2- methylmorpholino)- pyrimidin-5-yl)nicotinate | MS(ES): 500 (M + 1) for C₂₅H₂₇ClFN₅O₃ ¹H NMR (300 MHz, DMSO-D6) δ ppm 0.60 (t, J = 7.44 Hz, 3 H) 1.01 (d, J = 6.03 Hz, 3 H) 1.34 (t, J = 7.06 Hz, 3 H) 1.45- 1.89 (m, 2 H) 2.63-2.96 (m, 1 H) 3.33-3.76 (m, 5 H) 4.37 (q, J = 7.10 Hz, 2 H) 7.36 (t, J = 9.14 Hz, 1 H) 7.46-7.68 (m, 1 H) 8.04-8.20 (m, 2 H) 8.32 (t, J = 2.17 Hz, 1 H) 8.90 (d, J = 2.26 Hz, 1 H) 9.04 (d, J = 1.88 Hz, 1 H) 9.84 (s, 1 H) | 5-Bromo-N-(3- chloro-4- fluorophenyl)-4-(5- ethyl-2- methylmorpholino) pyrimidin-2-amine Intermediate 384 and 3- (ethoxycarbonyl)- pyridine-5-boronic acid pinacol ester |
| Example 982 | 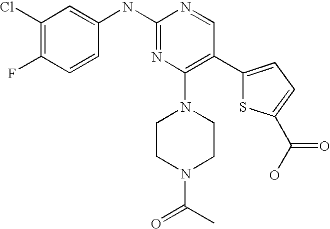 5-(4-(4-Acetylpiperazin-1- yl)-2-(3-chloro-4- fluorophenylamino)- pyrimidin-5-yl)thiophene-2- carboxylic acid | MS(ES): 476 (M + 1) for C₂₁H₁₉ClFN₅O₃S ¹H NMR (300 MHz, DMSO-D6) δ ppm 1.98 (s, 3 H) 3.22-3.41 (m, 4 H) 3.40-3.66 (m, 4 H) 7.27 (d, J = 3.77 Hz, 1 H) 7.35 (t, J = 9.14 Hz, 1 H) 7.53-7.68 (m, 1 H) 7.70 (d, J = 3.77 Hz, 1 H) 7.96- 8.17 (m, 1 H) 8.22 (s, 1 H) 9.84 (s, 1H) | 1-(4-(5-bromo-2- (3-chloro-4- fluorophenyl- amino)pyrimidin-4- yl)piperazin-1- yl)ethanone Intermediate 98 and 2- carboxythiophene- 5-boronic acid |
| Example 983 | 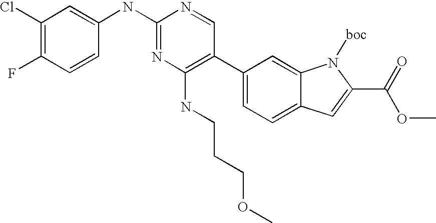 1-tert-butyl 2-methyl-6-(2- (3-chloro-4- fluorophenylamino)-4-(3- methoxypropylamino)- pyrimidin-5-yl)-1H-indole-1,2- dicarboxylate | MS(ES): 584 (M + 1) for C₂₉H₃₁ClFN₅O₅ ¹H MR (300 MHz, DMSO-D6) δ ppom 1.55 (s, 9 H) 1.69-2.00 (m, 2 H) 3.15 (s, 3 H) 3.35- 3.61 (m, 4 H) 3.87 (s, 3 H) 6.82 (t, J = 5.27 Hz, 1 H) 7.20-7.39 (m, 3 H) 7.53-7.71 (m, 1 H) 7.74- 7.90 (m, 2 H) 7.99 (s, 1 H)8.25 (dd, J = 6.88, 2.54 Hz, 1 H) 9.40 (s, 1 H) | 5-bromo-N²-(3- chloro-4- fluorophenyl)-N⁴- (3- methoxypropyl)- pyrimidin-2,4- diamine Intermediate 119 and 1-tert-butyl 2- methyl 6-(4,4,5,5- tetramethyl-1,3,2- dioxaborolan-2- yl)-1H-indole- 1,2-dicarboxylate Intermediate 408 |
| Example 984 | 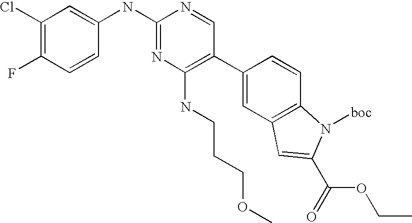 1-tert-butyl 2-ethyl 5-(2-(3- chloro-4- fluorophenylamino)-4-(3- methoxypropylamino)- pyrimidin-5-yl)-1H-indole-1,2- dicarboxylate | MS(ES): 598 (M + 1) for C₃₀H₃₃ClFN₅O₅ ¹H NMR (300 MHz, DMSO-D6) δ ppm 1.32 (t, J = 7.06 Hz, 3 H) 1.58 (s, 9 H) 1.79 (t, J = 6.41 Hz, 2 H) 3.15 (s, 3 H) 3.32-3.54 (m, 4 H) 4.33 (q, J = 7.16 Hz, 2 H) 6.60 (t, J = 5.27 Hz, 1 H) 7.16- 7.38 (m, 2H) 7.44 (dd, J = 8.67, 1.70 Hz, 1 H) 7.56-7.75 (m, 2 H) 7.79 (s, 1 H) 8.02 (d, J = 8.67 Hz, 1 H) 8.24 (dd, J = 6.97, 2.45 Hz, 1 H) 9.37 (s, 1 H) | 5-bromo-N²-(3- chloro-4- fluorophenyl)-N⁴- (3- methoxypropyl)- pyrimidin-2,4- diamine Intermediate 119 and 1-tert-butyl 2- ethyl 5-(4,4,5,5- tetramethyl)-1,3,2- dioxaborolan-2- yl)-1H-indole- 1,2-dicarboxylate Intermediate 409 |
| Example 985 | 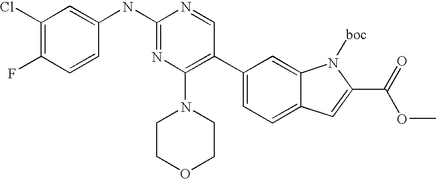 1-tert-butyl 2-methyl-6-(2- (3-choloro-4- fluorophenylamino)-4- morpholinopyrimidin-5- yl)-1H-indole-1,2- dicarboxylate | MS(ES): 582 (M + 1) for C₂₉H₂₉ClFN₅O₅ ¹H NMR (300 MHz, DMSO-D6) δ ppm 1.56 (s, 9 H) 3.11-3.27 (m, 4 H) 3.43-3.62 (m, 4 H) 3.87 (s, 3 H) 7.19-7.38 (m, 2 H) 7.44 (dd, J = 8.29, 1.32 Hz, 1 H) 7.54-7.72 (m, 1 H) 7.77 (d, J = 8.10 Hz, 1 H) 8.03- 8.23 (m, 3 H) 9.65 (s, 1 H) | 5-Bromo-N-(3- chloro-4- fluorophenyl)-4- morpholin-4- ylpyrimidin-2- amine Intermediate 111 and 1-tert-butyl 2- methyl 6-(4,4,5,5- tetramethyl-1,3,2- dioxaborolan-2- yl)-1H-indole- 1,2-dicarboxylate Intermediate 408 |
| Example 986 | 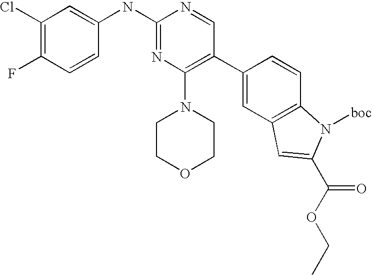 1-tert-butyl 2-ethyl 5-(2-(3- chloro-4- fluorophenylamino)-4- morpholinopyrimidin-5- yl)-1H-indole-1,2- dicarboxylate | MS(ES): 596 (M + 1) for C₃₀H₃₁ClFN₅O₅ ¹H NMR (300 MHz, DMSO-D6) δ ppm 1.32 (t, J = 7.06 Hz, 3 H) 1.58 (s, 9 H) 3.14-3.27 (m, 4 H) 3.43-3.69 (m, 4 H) 4.32 (q, J = 7.10 Hz, 2 H) 7.17-7.42 (m, 2 H) 7.47- 7.71 (m, 2 H) 7.78 (d, J = 1.32 Hz, 1 H) 7.95- 8.10 (m, 2 H) 8.15 (dd, J = 6.78, 2.64 Hz, 1 H) 9.60 (s, 1 H) | 5-Bromo-N-(3- chloro-4- fluorophenyl)-4- morpholin-4- ylpyrimidin-2- amine Intermediate 111 and 1-tert-butyl 2- ethyl 5-(4,4,4,5,5- tetramethyl-1,3,2- dioxaborolan-2- yl)-1H-indole- 1,2-dicarboxylate Intermediate 409 |
| Example 987 | 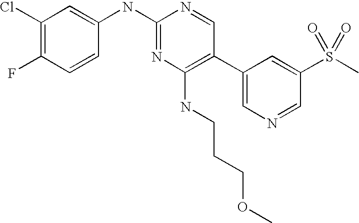 N2-(3-chloro-4- fluorophenyl)-N4-(3- methoxypropyl)-5-(5- (methylsulfonyl)pyridin-3- yl)pyrimidine-2,4-diamine | MS(ES): 466 (M + 1) for C₂₀H₂₁ClFN₅O₃S ¹H NMR (300 MHz, DMSO-D6) δ ppm 1.71- 1.93 (m, 2 H) 3.20 (s, 3 H) 3.33-3.55 (m, 7 H) 7.09 (t, J = 5.56 Hz, 1H) 7.30 (t, J = 9.14 Hz, 1 H) 7.53-7.74 (m, 1 H) 7.91 (s, 1 H) 8.16-8.35 (m, 2 H) 8.88 (d, J = 2.07 Hz, 1 H) 9.01 (d, J = 2.07 Hz, 1 H) 9.53 (s, 1 H) | 5-Bromo-N²-(3- chloro-4- fluorophenyl)-N⁴- (3- methoxypropyl)- pyrimidin-2,4- diamine Intermediate 119 and 3- (methylsulfonyl)- 5-(4,4,5,5- tetramethyl-1,3,2- dioxaborolan-2- yl)pyridine Intermediate 410 |
| Example 988 | 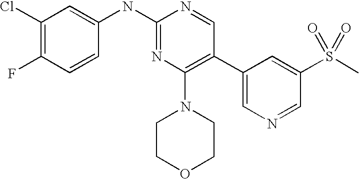 N-(3-chloro-4- fluorophenyl)-5-(5- (methylsulfonyl)pyridin-3- yl)-4- morpholinopyrimidin-22- amine | MS(ES): 464 (M + 1) for C₂₀H₁₉ClFN₅O₃S ¹H NMR (300 MHz, DMSO-D6) δ ppm 3.10- 3.28 (m, 4 H) 3.37 (s, 3 H) 3.49-3.73 (m, 4 H) 7.34 (t, J = 9.14 Hz, 1 H) 7.52-7.78 (m, 1 H) 8.04- 8.20 (m, 1 H) 8.25 (s, 1 H) 8.41 (s, 1 H) 9.00 (dd, J = 16.48, 1.41 Hz, 2 H) 9.79 (s, 1 H) | 5-Bromo-N-(3- chloro-4- fluorophenyl)-4- morpholin-4- ylpyrimidin-2- amine Intermediate 111 and 3- (methylsulfonyl)- 5-(4,4,5,5- tetramethyl-1,3,2- dioxaborolan-2- yl)pyridine Intermediate 410 |
| Example 989 | 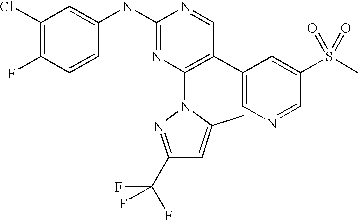 N-(3-chloro-4- fluorophenyl)-4-(5-methyl- 3-(trifluoromethyl)-1H- pyrazol-1-yl)-5-(5- (methylsulfonyl)pyridin-3- yl)pyrimidin-2-amine | MS(ES): 527 (M + 1) for C₂₁H₁₅ClF₄N₆O₂S ¹H NMR (300 MHz, DMSO-D6) δ ppm 3.24 (s, 3 H) 3.30 (s, 3 H) 6.80 (s, 1 H) 7.43 (t, J = 9.14 Hz, 1 H) 7.58-7.75 (m, 1 H) 7.94 (t, J = 2.17 Hz, 1 H) 8.07 (dd, J = 6.59, 2.26 Hz, 1 H) 8.62 (d, J = 2.07 Hz, 1 H) 8.92-9.09 (m, 2 H) 10.51 (s, 1 H) | 5-bromo-N-(3- chloro-4- fluorophenyl)-4- [5-methyl-3- (trifluoromethyl)- 1H-pyrazol-1- yl]pyrimidin-2- amine Intermediate 113 and 3- (methylsulfonyl)- 5-(4,4,5,5- tetramethyl-1,3,2- dioxaborolan-2- yl)pyridine Intermediate 410 |
| Example 990 | 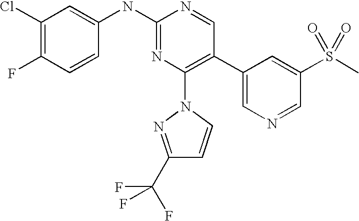 N-(3-chloro-4- fluorophenyl)-5-(5- (methylsulfonyl)pyridin-3- yl)-4-(3-(trifluoromethyl)- 1H-pyrazol-1- yl)pyrimidin-2-amine | MS(ES): 513 (M + 1) for C₂₀H₁₃ClF₄N₆O₂S ¹H NMR (300 MHz, DMSO-D6) δ ppm 3.27 (s, 3 H) 7.07 (d, J = 2.64 Hz, 1 H) 7.43 (t, J = 9.04 Hz, 1 H) 7.63-7.80 (m, 1 H) 8.06 (dd, J = 6.78, 2.45 Hz, 1 H) 8.15 (t, J = 2.07 Hz, 1 H) 8.59 (d, J = 1.70 Hz, 1 H) 8.76 (d, J = 2.07 Hz, 1 H) 8.85 (s, 1 H) 9.02 (d, J = 2.26 Hz, 1 H) 10.50 (s, 1 H) | 5-bromo-N-(3- chloro-4- fluorophenyl)-4- [3-(trifluoro- methyl)-1H- pyrazol-1- yl]pyrimidin-2- amine Intermediate 115 and 3- (methylsulfonyl)- 5-(4,4,5,5- tetramethyl-1,3,2- dioxaborolan-2- yl)pyridine Intermediate 410 |
| Example 991 | 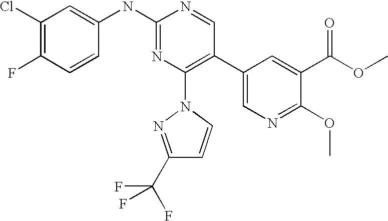 Methyl 5-(2-(3-chloro-4- fluorophenylamino)-4-(3- (trifluoromethyl)-1H- pyrazol-1-yl)pyrimidin-5- yl)-2-methoxynicinate | MS(ES): 523 (M + 1) for C₂₂H₁₅ClF₄N₆O₃ ¹H NMR (300 MHz, DMSO-d₆) δ ppm 3.77 (s, 3 H) 3.95 (s, 3 H) 7.05 (d, J = 2.64 Hz, 1 H) 7.43 (t, J = 9.14 Hz, 1 H) 7.72 (ddd, J = 9.04, 4.24, 2.73 Hz, 1 H) 7.86 (d, J = 2.45 Hz, 1 H) 8.08 (dd, J = 6.69, 2.54 Hz, 1 H) 8.28 (d, J = 1.51 Hz, 1 H) 8.81 (s, 1 H) 10.42 (s, 1 H) | 5-bromo-N-(3- chloro-4- fluorophenyl)-4- [3-(trifluoro- methyl)-1H- pyrazol-1- yl]pyrimidin-2- amine Intermediate 115 and methyl 2- methoxy-5- (4,4,5,5- tetramethyl-1,3,2- dioxaborolan-2- yl)nicotinate Intermediate 411 |
| Example 992 | 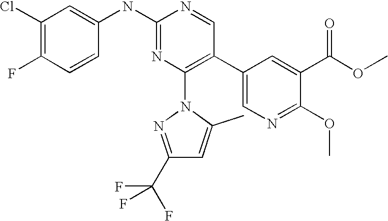 Methyl 5-(2-(3-chloro-4- fluorophenylamino)-4-(5- methyl-3-(trifluoromethyl)- 1H-pyrazol-1- yl)pyrimidin-5-yl)-2- methoxynicotinate | MS(ES): 537 (M + 1) for C₂₃H₁₇ClF₄N₆O₃ ¹H NMR (300 MHz, DMSO-d₆) δ ppm 2.33 (s, 3 H) 3.75 (s, 3 H) 3.92 (s, 3 H) 6.77 (s, 1 H) 7.42 (t, J = 9.14 Hz, 1 H) 7.54- 7.76 (m, 2 H) 8.07 (d, J = 6.97 Hz, 1 H) 8.23 (d, J = 2.45 Hz, 1 H) 8.97 (s, 1 H) 10.46 (s, 1 H) | 5-bromo-N-(3- chloro-4- fluorophenyl)-4- [5-methyl-3- (trifluoromethyl)- 1H-pyrazol-1- yl]pyrimidin-2- amine Intermediate 113 and methyl 2- methoxy-5- (4,4,5,5- tetramethyl-1,3,2- dioxaborolan-2- yl)nicotinate Intermediate 411 |
| Example 993 | 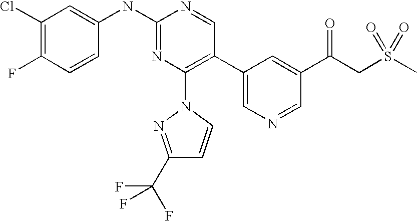 1-(5-(2-(3-chloro-4- fluorophenylamino)-4-(3- (trifluoromethyl)-1H- pyrazol-1-yl)pyrimidin-5- yl)pyridin-3-yl)-2- (methylsulfonyl)ethanone | MS(ES): 555 (M + 1) for C₂₂H₁₅ClF₄N₆O₃S ¹H NMR (300 MHz, DMSO-d₆) δ ppm 3.16 (s, 3 H) 5.19 (s, 2 H) 7.07 (d, J = 2.64 Hz, 1 H) 7.44 (t, J = 9.14 Hz, 1 H) 7.74 (ddd, J = 9.14, 4.24, 2.83 Hz, 1 H) 8.08 (dd, J = 6.69, 2.54 Hz, 1 H) 8.31 (t, J = 2.17 Hz, 1 H) 8.49-8.70 (m, 2 H) 8.85 (s, 1 H) 9.15 (d, J = 2.07 Hz, 1 H) 10.50 (s, 1 H) | 5-bromo-N-(3- chloro-4- fluorophenyl)-4- [3-(trifluoro- methyl)-1H- pyrazol-1- yl]pyrimidin-2- amine Intermediate 115 and 2- (Methylsulfonyl)- 1-(5-(4,4,5,5- tetramethyl-1,3,2- dioxaborolan-2- yl)pyridin-3- yl)ethanone Intermediate 415 |
| Example 994 | 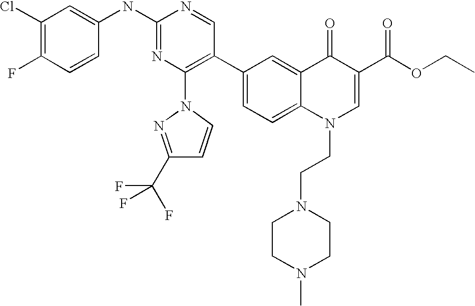 Ethyl 6-(2-(3-chloro-4- fluorophenylamino)-4-(3- (trifluoromethyl)-1H- pyrazol-1-yl)pyrimidin-5- yl)-1-(2-(4- methylpiperazin-1- yl)ethyl)-4-oxo-1,4- dihydroquinoline-3- carboxylate | MS(ES): 699 (M + 1) for C₃₃H₃₁ClF₄N₈O₃ ¹H NMR (300 MHz, DMSO-d₆) δ ppm 1.30 (t, J = 7.06 Hz, 3 H) 2.01- 2.46 (m, 11 H) 2.60 (t, J = 4.80 Hz, 2 H) 4.23 (q, J = 7.10 Hz, 2 H) 4.47 (t, J = 4.24 Hz, 2 H) 7.00 (d, J = 2.64 Hz, 1 H) 7.33- 7.56 (m, 2 H) 7.60-7.88 (m, 2 H) 8.00-8.19 (m, 2 H) 8.44 (d, J = 1.51 Hz, 1 H) 8.58 (s, 1 H) 8.83 (s, 1 H) 10.44 (s, 1 H) | 5-bromo-N-(3- chloro-4- fluorophenyl)-4- [3-(trifluoro- methyl)-1H- pyrazol-1- yl]pyrimidin-2- amine Intermediate 115 and Ethyl 1-(2-(4- methylpiperazin- 1-yl)ethyl)-4-oxo- 6-(4,4,5,5- tetramethyl-1,3,2- dioxaborolan-2- yl)-1,4- dihydroquinoline- 3-carboxylate Intermediate 416 |
| Example 995 | 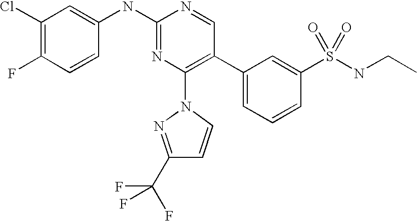 3-(2-(3-Chloro-4- fluorophenylamino)-4-(3- (trifluoromethyl)-1H- pyrazol-1-yl)pyrimidin-5- yl)-N- ethylbenzenesulfonamide | MS(ES): 541 (M + 1) for C₂₂H₁₇ClF₄N₆O₂S ¹H NMR (300 MHz, DMSO-d₆) δ ppm 0.91- 1.01 (m, 3 H) 2.56-2.81 (m, 2 H) 7.00 (d, J = 2.64 Hz, 1 H) 7.29-7.66 (m, 5 H) 7.65-7.90 (m, 2 H) 8.09 (dd, J = 6.78, 2.64 Hz, 1 H) 8.45 (d, J = 1.51 Hz, 1 H) 8.77 (s, 1 H) 10.42 (s, 1 H) | 5-bromo-N-(3- chloro-4- fluorophenyl)-4- [3-(trifluoro- methyl)-1H- pyrazol-1- yl]pyrimidin-2- amine Intermediate 115 and ethyl 3- boronobenzene- sulfonamide |
| Example 996 | 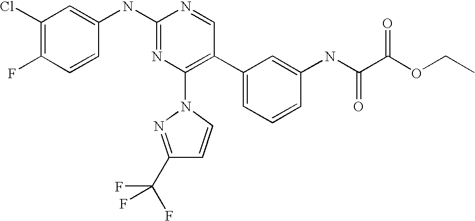 Ethyl 2-(3-(2-(3-chloro-4- fluorophenylamino)-4-(3- (trifluoromethyl)-1H- pyrazol-1-yl)pyrimidin-5- yl)phenylamino)-2- oxoacetate | MS(ES): 549 (M + 1) for C₂₄H₁₇ClF₄N₆O₃ ¹H NMR (300 MHz, DMSO-d₆) δ ppm 1.31 (t, J = 7.06 Hz, 3 H) 4.30 (q, J = 7.16 Hz, 2 H) 6.81- 7.07 (m, 2 H) 7.21-7.48 (m, 2 H) 7.63 (s, 1 H) 7.66-7.81 (m, 2 H) 8.12 (dd, J = 6.78, 2.64 Hz, 1 H) 8.29 (s, 1 H) 8.73 (s, 1 H) 10.40 (s, 1 H) 10.77 (s, 1 H) | 5-bromo-N-(3- chloro-4- fluorophenyl)-4- [3-(trifluoro- methyl)-1H- pyrazol-1- yl]pyrimidin-2- amine Intermediate 115 and ethyl 2-oxo-2-(3- (4,4,5,5- tetramethyl-1,3,2- dioxaborolan-2- yl)phenylamino)- acetate Intermediate 418 |
| Example 997 | 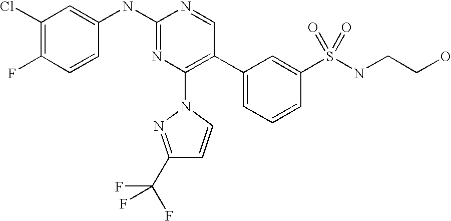 3-(2-(3-Chloro-4- fluorophenylamino)-4-(3- (trifluoromethyl)-1H- pyrazol-1-yl)pyrimidin-5- yl)-N-(2- hydroxyethyl)benzene- sulfonamide | MS(ES): 557 (M + 1) for C₂₂H₁₇ClF₄N₆O₃S ¹H NMR (300 MHz, DMSO-d₆) δ ppm 2.71 (q, 2 H) 3.36 (q, J = 6.03 Hz, 2 H) 4.66 (t, J = 5.56 Hz, 1 H) 6.99 (d, J = 2.83 Hz, 1 H) 7.30-7.49 (m, 2 H) 7.49-7.67 (m, 3 H) 7.67-7.90 (m, 2 H) 8.10 (dd, J = 6.78, 2.64 Hz, 1 H) 8.43 (d, J = 1.70 Hz, 1 H) 8.78 (s, 1 H) 10.43 (s, 1 H) | 5-bromo-N-(3- chloro-4- fluorophenyl)-4- [3-(trifluoro- methyl)-1H- pyrazol-1- yl]pyrimidin-2- amine Intemdiate 115 and 3-(N-(2- hydroxyethyl)- sulfamoyl)- phenylboronic acid |
| Example 998 | 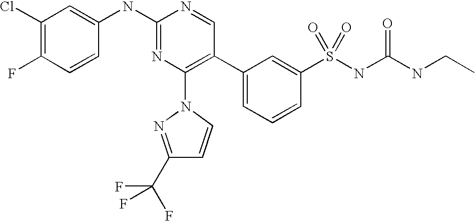 3-(2-(3-Chloro-4- fluorophenylamino)-4-(3- (trifluoromethyl)-1H- pyrazol-1-yl)pyrimidin-5- yl)-N- (ethylcarbamoyl)benzene- sulfonamide | MS(ES): 584 (M + 1) for C₂₃H₁₈ClF₄N₇O₃S ¹H NMR (300 MHz, DMSO-d₆) δ ppm 0.92 (t, 3 H) 2.85-3.04 (m, 2 H) 6.41 (t, J = 5.93 Hz, 1 H) 6.99 (d, J = 2.45 Hz, 1 H) 7.26-7.67 (m, 5 H) 7.73 (ddd, J = 9.09, 4.29, 2.64 Hz, 1 H) 7.79-7.97 (m, 1 H) 8.10 (dd, J = 6.78, 2.64 Hz, 1 H) 8.43 (s, 1 H) 8.76 (s, 1 H) 10.46 (s, 1 H) | 5-bromo-N-(3- chloro-4- fluorophenyl)-4- [3-(trifluoro- methyl)-1H- pyrazol-1- yl]pyrimidin-2- amine Intermediate 115 and N- (ethylcarbamoyl)- 3-(4,4,5,5- tetramethyl-1,3,2- dioxaborolan-2- yl)benzenesulfon- amide Intermediate 419 |
| Example 999 | 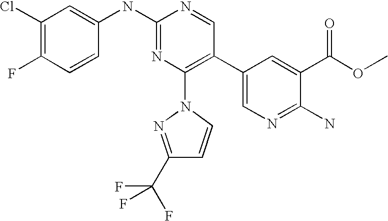 Methyl 2-amino-5-(2-(3- chloro-4- fluorophenylamino)-4-(3- (trifluoromethyl)-1H- pyrazol-1-yl)pyrimidin-5- yl)nicotinate | MS(ES): 508 (M + 1) for C₂₁H₁₄ClF₄N₇O₂ ¹H NMR (300 MHz, DMSO-d₆) δ ppm 3.77 (s, 3 H) 7.02 (d, J = 2.64 Hz, 1 H) 7.28 (s, 2 H) 7.40 (t, J = 9.04 Hz, 1 H) 7.62-7.82 (m, 2 H) 7.98- 8.18 (m, 2 H) 8.43 (d, J = 1.70 Hz, 1 H) 8.78 (s, 1 H) 10.35(s, 1 H) | 5-bromo-N-(3- chloro-4- fluorophenyl)-4- [3-(tyrifluoro- methyl)-1H- pyrazol-1- yl]pyrimidin-2- amine Intermediate 115 and methyl 2-amino- 5-(4,4,5,5- tetramethyl-1,3,2- dioxaborolan-2- yl)nicotinate Intermediate 420 |
| Example 1000 | 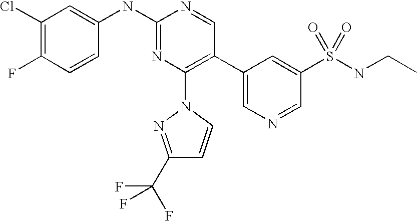 5-(2-(3-Chloro-4- fluorophenylamino)-4-(3- (trifluoromethyl)-1H- pyrazol-1-yl)pyrimidin-5- yl)-N-ethylpyridine-3- sulfonamide | MS(ES): 542 (M + 1) for C₂₁H₁₆ClF₄N₇O₂S ¹H NMR (300 MHz, DMSO-d₆) δ ppm 0.97 (t, 3 H) 2.63-2.86 (m, 2 H) 7.05 (d, J = 2.83 Hz, 1 H) 7.43 (t, J = 9.14 Hz, 1 H) 7.65-7.86 (m, 2 H) 7.97 (t, J = 2.07 Hz, 1 H) 8.06 (dd, J = 6.69, 2.54 Hz, 1 H) 8.58 (dd, J = 2.54, 0.85 Hz, 1 H) 8.67 (d, J = 2.07 Hz, 1 H) 8.81 (s, 1 H) 8.89 (d, J = 2.07 Hz, 1 H) 10.46 (s, 1 H) | 5-Bromo-N-(3- chloro-4- fluorophenyl)-4- [3-(trifluoro- methyl)-1H- pyrazol-2- yl]pyrimidin-2- amine Intermediate 115 and N-ethyl-5- (4,4,5,5- tetramethyl-1,3,2- dioxaborolan-2- yl)pyridine-3- sulfonamide Intermediate 421 |
| Example 1001 | 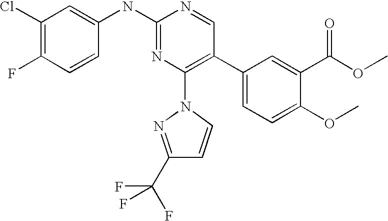 Methyl 5-(2-(3-chloro-4- fluorophenylamino)-4-(3- (trifluoromethyl)-1H- pyrazol-1-yl)pyrimidin-5- yl)-2-methoxybenzoate | MS(ES): 522 (M + 1) for C₂₃H₁₆ClF₄N₅O₃ ¹H NMR (300 MHz, DMSO-d₆) δ ppm 3.74 (s, 3 H) 3.83 (s, 3 H) 6.98 (d, J = 2.64 Hz, 1 H) 7.07- 7.19 (m, 1 H) 7.31- 7.38 (m, 2 H) 7.70 (ddd, J = 9.14, 4.14, 2.73 Hz, 1 H) 7.77-7.94 (m, 1 H) 8.11 (dd, J = 6.78, 2.64 Hz, 1 H) 8.32 (d, J = 1.70 Hz, 1 H) 8.77 (s, 1 H) 10.37 (s, 1 H) | 5-Bromo-N-(3- chloro-4- fluorophenyl)-4- [3-(trifluoro- methyl)-1H- pyrazol-1- yl]pyrimidin-2- amine Intermediate 115 and methyl 2- methoxy-5- (4,4,5,5- tetramethyl-1,3,2- dioxaborolan-2- yl)benzoate Intermediate 422 |
| Example 1002 | 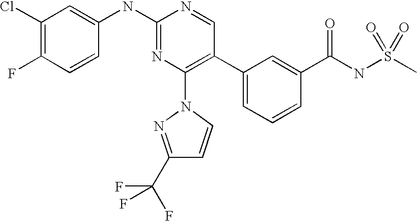 3-(2-(3-Chloro-4- fluorophenylamino)-4-(3- (trifluoromethyl)-1H- pyrazol-1-yl)pyrimidin-5- yl)-N- (methylsulfonyl)benzamide | MS(ES): 555 (M + 1) for C₂₂H₁₅ClF₄N₆O₃S ¹H NMR (300 MHz, DMSO-d₆) δ ppm 3.38 (s, 3 H) 7.01 (d, J = 2.64 Hz, 1 H) 7.20-7.35 (m, 1 H) 7.35-7.61 (m, 2 H) 7.62-7.81 (m, 1 H) 7.81- 8.04 (m, 2 H) 8.13 (dd, J = 6.78, 2.64 Hz, 1 H) 8.39 (s, 1 H) 8.85 (s, 1 H) 10.44 (s, 1 H) 12.13 (br. s., 1 H) | 5-Bromo-N-(3- chloro-4- fluorophenyl)-4- [3-(trifluoro- methyl)-1H- pyrazol-1- yl]pyrimidiun-2- amine Intermediate 115 and N- (methylsulfonyl)- 3-(4,4,5,5- tetramethyl-1,3,2- dioxaborolan-2- yl)benzamide Intermediate 423 |
| Example 1003 | 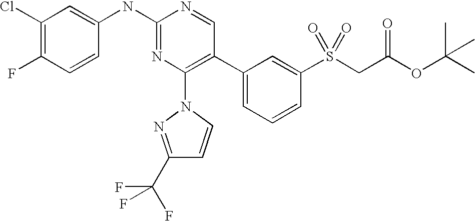 tert-Butyl 2-(3-(2-(3- chloro-4- fluorophenylamino)-4-(3- (trifluorophenylamino)-1H- pyrazol-1-yl)pyrimidin-5- yl)phenylsulfonyl)acetate | MS(ES): 612 (M + 1) for C₂₆H₂₂ClF₄N₅O₄S ¹H NMR (300 MHz, DMSO-d₆) δ ppm 1.27 (s, 9 H) 4.44 (s, 2 H) 7.02 (d, J = 2.64 Hz, 1 H) 7.42 (t, J = 9.14 Hz, 1 H) 7.48- 7.58 (m, 1 H) 7.60-7.81 (m, 3 H) 7.79-7.96 (m, 1 H) 8.10 (dd, J = 6.78, 2.64 Hz, 1 H) 8.46 (d, J = 1.51 Hz, 1 H) 8.81 (s, 1 H) 10.46 (s, 1 H) | 5-Bromo-N-(3- chloro-4- fluorophenyl)-4- [3-(trifluoro- methyl)-1H- pyrazol-1- yl]pyrimidin-2- amine Intermediate 115 and tert-butyl 2-(3- (4,4,5,5- tetramethyl-1,3,2- dioxaborolan-2- yl)phenylsulfonyl)- acetate Intermediate 424 |
| Example 1004 | 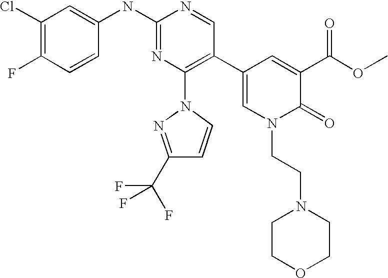 Methyl 5-(2-(3-chloro-4- fluorophenylamino)-4-(3- (trifluoromethyl)-1H- pyrazol-1-yl)pyrimidin-5- yl)-1-(2-morpholinoethyl)- 2-oxo-1,2-dihydropyridine- 3-carboxylate | MS(ES): 622 (M + 1) for C₂₇H₂₄ClF₄N₇O₄ ¹H NMR (300 MHz, DMSO-d₆) δ ppm 2.34- 2.45 (m, 4 H) 2.56 (t, J = 6.40 Hz, 2 H) 3.41- 3.59 (m, 4 H) 3.68 (s, 3 H) 3.97-4.11 (m, 2 H) 7.08 (d, J = 2.83 Hz, 1 H) 7.42 (t, J = 9.04 Hz, 1 H) 7.59-7.80 (m, 2 H) 7.94- 8.16 (m, 2 H) 8.48- 8.63 (m, 1 H) 8.71 (s, 1 H) 10.36 (s, 1 H) | 5-Bromo-N-(3- chloro-4- fluorophenyl)-4- [3-(trifluoro- methyl)-1H- pyrazol-1- yl]pyrimidin-2- amine Intermediate 115 and methyl 1-(2- morpholinoethyl)- 2-oxo-5-(4,4,5,5- tetramethyl-1,3,2- dioxaborolan-2- yl)-1,2- dihydropyridine- 3-carboxylate Intermediate 425 |
| Example 1005 | 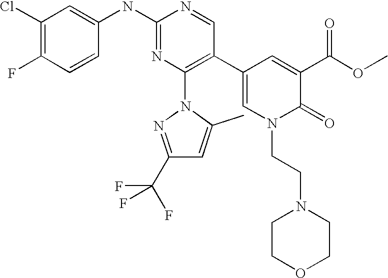 Methyl 5-(2-(3-chloro-4- fluorophenylamino)-4-(5- methyl-3-(trifluoromethyl)- 1H-pyrazol-1- yl)pyrimidin-5-yl)-1-(2- morpholinoethyl)-2-oxo- 1,2-dihydropyridine-3- carboxylate | MS(ES): 636 (M + 1) for C₂₈H₂₆ClF₄N₇O₄ ¹H NMR (300 MHz, DMSO-d₆) δ ppm 2.31- 2.46 (m, 7 H) 2.51-2.63 (m, 2 H) 3.40-3.60 (m, 4 H) 3.65 (s, 3 H) 3.94- 4.13 (m, 2 H) 6.79 (s, 1 H) 7.27 (d, J = 2.64 Hz, 1 H) 7.41 (t, J = 9.14 Hz, 1 H) 7.64 (ddd, J = 9.09, 4.19, 2.73 Hz, 1 H) 7.95- 8.16 (m, 2 H) 8.86 (s, 1 H) 10.40 (s, 1 H) | 5-Bromo-N-(3- chloro-4- fluorophenyl)-4- [5-methyl-3- (trifluoromethyl)- 1H-pyrazol-1- yl]pyrimidin-2- amine Intermediate 115 and Methyl 1-(2- morpholinoethyl)- 2-oxo-5-(4,4,5,5- tetramethyl-1,3,2- dioxaborolan-2- yl)-1,2- dihydropyridine- 3-carboxylate Intermediate 425 |
| Example 1006 | 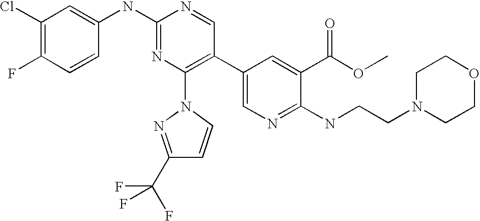 Methyl 5-(2-(3-chloro-4- fluorophenylamino)-4-(3- (trifluoromethyl)-1H- pyrazol-1-yl)pyrimidin-5- yl)-2-(2- morpholinoethylamino)- nicotinate | MS(ES): 621 (M + 1) for C₂₇H₂₅ClF₄N₈O₃ ¹H NMR (300 MHz, DMSO-d₆) δ ppm 2.32- 2.46 (m, 4 H) 2.45-2.63 (m, 2 H) 3.45-3.68 (m, 6 H) 3.77 (s, 3 H) 7.02 (d, J = 2.64 Hz, 1 H) 7.40 (t, J = 9.14 Hz, 1 H) 7.57- 7.83 (m, 2 H) 8.09 (dd, J = 6.78, 2.64 Hz, 1 H) 8.10-8.26 (m, 2 H) 8.43 (d, J = 1.32 Hz, 1 H) 8.72- 8.89 (m, 1 H) 10.35 (s, 1 H) | 5-Bromo-N-(3- chloro-4- fluorophenyl)-4- [3-(trifluoro- methyl)-1H- pyrazol-1- yl]pyrimidin-2- amine Intermediate 115 and methyl 2-(2- morpholinoethyl- amino)-5-(4,4,5,5- tetramethyl-1,3,2- dioxaborolan-2- yl)nicotinate Intermediate 426 |
| Example 1007 | 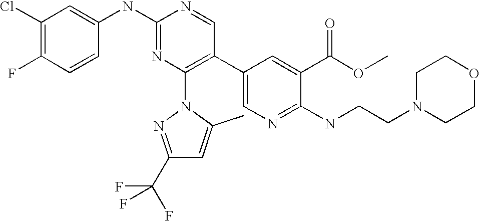 Methyl 5-(2-(3-chloro-4- fluorophenylamino)-4-(5- methyl-3-(trifluoromethyl)- 1H-pyrazol-1- yl)pyrimidin-5-yl)-2-(2- morpholinoethylamino)- nicotinate | MS(ES): (M + 1H) for C₂₈H₂₇ClF₄N₈O₃ ¹H NMR (300 MHz, DMSO-d₆) δ ppm, 2.18 (s, 3 H) 2.32-2.46 (m, 4 H) 2.47-2.60 (m, 2 H) 3.55-3.68 (m, 6 H) 3.75 (s, 3 H) 7.18-7.54 (m, 2 H) 7.65 (ddd, J = 9.14, 4.24, 2.64 Hz, 1 H) 8.06 (dd, J = 6.69, 2.54 Hz, 1 H) 8.12-8.27 (m, 3 H) 8.95 (s, 1 H) 10.39 (s, 1 H) | 5-bromo-N-(3- chloro-4- fluorophenyl)-4- [5-methyl-3- (trifluoromethyl)- 1H-pyrazol-1- yl]pyrimidin-2- amine Intermediate 113 and methyl 2-(2- morpholinoethyl- amino)-5-(4,4,5,5- tetramethyl-1,3,2- dioxaborolan-2- yl)nicotinate Intermediate 426 |
| Example 1008 | 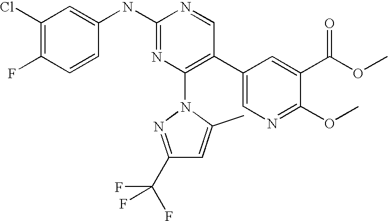 Methyl 5-(2-(3-chloro-4- fluorophenylamino)-4-(5- methyl-3-(trifluoromethyl)- 1H-pyrazol-1- yl)pyrimidin-5-yl)-2- methoxynicotinate | MS(ES): 537 (M + 1) for C₂₃H₁₇ClF₄N₆O₃ ¹H NMR (300 MHz, DMSO-d₆) δ ppm 2.33 (s, 3 H) 3.75 (s, 3 H) 3.92 (s, 3 H) 6.76 (s, 1 H) 7.42 (t, J = 9.14, Hz, 1 H) 7.53- 7.75 (m, 2 H) 8.06 (dd, J = 6.69, 2.54 Hz, 1 H) 8.22 (d, J = 2.64 Hz, 1 H) 8.96 (s, 1 H) 10.43 (s,1 H) | 5-bromo-N-(3- chloro-4- fluorophenyl)-4- [5-methyl-3- (trifluoromethyl)- 1H-pyrazol-1- yl]pyrimidin-2- amine Intermediate 113 and methyl 2- methoxy-5- (4,4,5,5- tetramethyl-1,3,2- dioxaborolan-2- yl)nicotinate Intermediate 411 |
| Example 1009 | 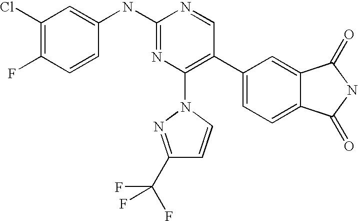 5-(2-(3-Chloro-4- fluorophenylamino)-4-(3- (trifluoromethyl)-1H- pyrazol-1-yl)pyrimidin-5- yl)isoindole-1,3-dione | MS(ES): 503 (M + 1) for C₂₂H₁₁ClF₄N₆O₂ ¹H NMR (300 MHz, DMSO-d₆) δ ppm 7.03 (d, 1 H) 7.43 (t, J = 9.04 Hz, 1 H) 7.54-7.69 (m, 2 H) 7.69-7.91 (m, 2 H) 8.09 (dd, J = 6.78, 2.45 Hz, 1 H) 8.49 (d, J = 1.70 Hz, 1 H) 8.83 (s, 1 H) 10.46 (s, 1 H) 11.37(s, 1 H) | 5-Bromo-N-(3- chloro-4- fluorophenyl)-4- [3-(trifluoro- methyl)-1H- pyrazol-1- yl]pyrimidin-2- amine Intermediate 115 and 5-(4,4,5,5- tetramethyl-1,3,2- dioxaborolan-2- yl)isoindole- 1,3-dione Intermediate 427 |
| Example 1010 | 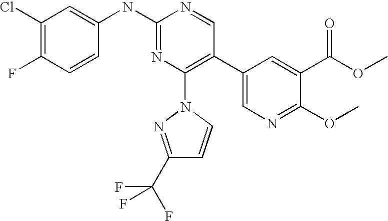 Methyl 5-(2-(3-chloro-4- fluorophenylamino)-4-(3- (trifluoromethyl)-1H- pyrazol-1-yl)pyrimidin-5- yl)-2-methoxynicotinate | MS(ES): 523 (M + 1) for C₂₂H₁₅ClF₄N₆O₃ ¹H NMR (300 MHz, DMSO-d₆) δ ppm 3.77 (s, 3 H) 3.95 (s, 3 H) 7.04 (d, J = 2.64 Hz, 1 H) 7.42 (d, J = 9.04 Hz,1 H) 7.62- 7.81 (m, 1 H) 7.86 (d, J = 2.45 Hz, 1 H) 8.07 (dd, J = 6.60, 2.54 Hz, 1 H) 8.28 (d, J = 2.45 Hz, 1 H) 8.51 (s, 1 H) 8.80 (s, 1 H) 10.39 (s, 1 H) | 5-Bromo-N-(3- chloro-4- fluorophenyl)-4- [3-(trifluoro- methyl)-1H- pyrazol-1- yl]pyrimidin-2- amine Intermediate 115 and methyl 2- methoxy-5- (4,4,5,5- tetramethyl-1,3,2- dioxborolan-2- yl)nicotinate Intermediate 411 |
| Example 1011 | 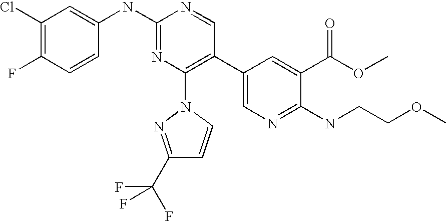 Methyl 5-(2-(3-chloro-4- fluorophenylamino)-4-(3- (trifluoromethyl)-1H- pyrazol-1-yl)pyrimidin-5- yl)-2-(2- methoxyethylamino)- nicotinate | MS(ES): 566 (M + 1) for C₂₄H₂₀ClF₄N₇O₃ ¹H NMR (300 MHz, DMSO-d₆) δ ppm 3.30 (s, 3 H) 3.43-3.57 (m, 2 H) 3.65 (q, J = 5.27 Hz, 2 H) 3.77 (s, 3 H) 7.02 (d, J = 2.64 Hz, 1 H) 7.40 (t, J = 9.14 Hz, 1 H) 7.63- 7.83 (m, 2 H) 8.02-8.14 (m, 2 H) 8.17 (d, J = 2.45 Hz, 1 H) 8.44 (d, J = 1.70 Hz, 1 H) 8.79 (s, 1 H) 10.35 (s, 1 H) | 5-Bromo-N-(3- chloro-4- fluorophenyl)-4- [3-(trifluoro- methyl)-1H- pyrazol-1- yl]pyrimidin-2- amine Intermediate 115 and methyl 2-(2- methoxyethyl- amino)-5-(4,4,5,5- tetramethyl-1,3,2- dioxaborolan-2- yl)nicotinate Intermediate 428 |
| Example 1012 | 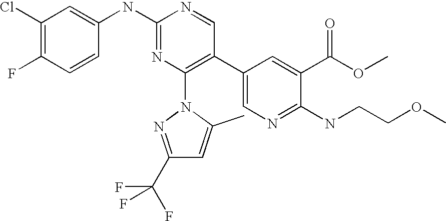 Methyl 5-(2-(3-chloro-4- fluorophenylamino)-4-(5- methyl-3-(trifluoromethyl)- 1H-pyrazol-1- yl)pyrimidin-5-yl)-2-(2- methoxyethylamino)- nicotinate | MS(ES): 580 (M + 1) for C₂₅H₂₂ClF₄N₇O₃ ¹H NMR (300 MHz, DMSO-d₆) δ ppm 2.20 (s, 3 H) 3.28 (s, 3 H) 3.42- 3.56 (m, 2 H) 3.62 (q, J = 5.21 Hz, 2 H) 3.75 (s, 3 H) 6.74 (s, 1 H) 7.40 (t, J = 9.14 Hz, 1 H) 7.50 (d, J = 2.45 Hz, 1 H) 7.65 (ddd, J = 9.04, 4.24, 2.73 Hz, 1 H) 7.99-8.15 (m, 2 H) 8.19 (d, J = 2.45 Hz, 1 H) 8.96 (s, 1 H) 10.39 (s, 1 H) | 5-bromo-N-(3- chloro-4- fluorophenyl)-4- [5-methyl-3- (trifluoromethyl)- 1H-pyrazol-1- yl]pyrimidin-2- amine Intermediate 113 and methyl 2-(2- methoxyethyl- amino)-5-(4,4,5,5- tetramethyl-1,3,2- dioxaborolan-2- yl)nicotinate Intermediate 428 |
| Example 1013 | 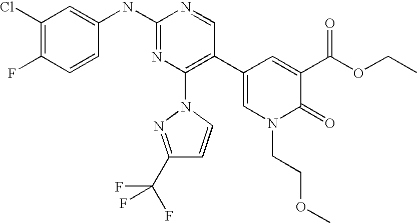 Ethy 5-(2-(3-chloro-4- fluorophenylamino)-4-(3- (trifluoromethyl)-1H- pyrazol-1-yl)pyrimidin-5- yl)-1-(2-methoxyethyl)-2- oxo-1,2-dihydropyridine-3- carboxylate | MS(ES): 581 (M + 1) for C₂₅H₂₁ClF₄N₆O₄ ¹H NMR (300 MHz, DMSO-d₆) δ ppm 1.19 (t, J = 7.16 Hz, 3 H) 3.23 (s, 3 H) 3.50-3.67 (m, 2 H) 3.96-4.21 (m, 4 H) 7.07 (d, J = 2.83 Hz, 1 H) 7.41 (t, J = 9.14 Hz, 1 H) 7.56- 7.79 (m, 2 H) 7.97-8.12 (m, 2 H) 8.55 (d, J = 1.70 Hz, 1 H) 8.71 (s, 1 H) 10.36 (s, 1 H) | 5-Bromo-N-(3- chloro-4- fluorophenyl)-4- [3-(trifluoro- methyl)-1H- pyrazol-1- yl]pyrimidin-2- amine Intermediate 115 and ethyl 1-(2- methoxyethyl)-2- oxo-5-(4,4,5,5- tetramethyl-1,3,2- dioxaborolan-2- yl)-1,2- dihydropyridine- 3-carboxylate Intermediate 429 |
| Example 1014 | 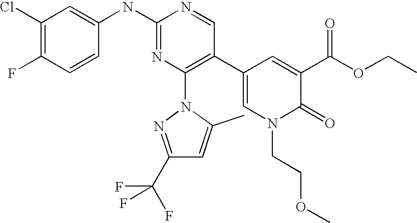 Ethyl 5-(2-(3-chloro-4- fluorophenylamino)-4-(5- methyl-3-(trifluoromethyl)- 1H-pyrazol-1- yl)pyrimidin-5-yl)-1-(2- methoxyethyl)-2-oxo-1,2- dihydropyridine-3- carboxylate | MS(ES): 595 (M + 1) for C₂₆H₂₃ClF₄N₆O₄ ¹H NMR (300 MHz, DMSO-d₆) δ ppm 1.19 (t, J = 7.06 Hz, 3 H) 2.37 (s, 3 H) 3.22 (s, 3 H) 3.57 (t, J = 5.37 Hz, 2 H) 3.96- 4.24 (m, 4 H) 6.79 (s, 1 H) 7.21-7.54 (m, 2 H) 7.64 (ddd, J = 9.04, 4.14, 2.64 Hz, 1 H) 8.03 (ddd, J = 11.63, 6.55, 2.54 Hz, 2 H) 8.84 (s, 1 H) 10.40 (s, 1 H) | 5-bromo-N-(3- chloro-4- fluorophenyl)-4- [5-methyl-3- (trifluoromethyl)- 1H-pyrazol-1- yl]pyrimidin-2- amine Intermediate 113 and ethyl 1-(2- methoxyethyl)-2- oxo-5-(4,4,5,5- tetramethyl-1,3,2- dioxaborolan-2- yl)-1,2- dihydropyridine- 3-carboxylate Intermediate 429 |
| Example 1015 | 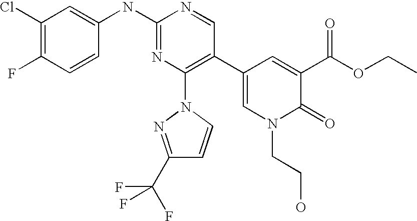 Ethyl 5-(2-(3-chloro-4- fluorophenylamino)-4-(3- (trifluoroethyl)-1H- pyrazol-1-yl)pyrimidin-5- yl)-1-(2-hydroxyethyl)-2- oxo-1,2-dihydropyridine-3- carboxylate | MS(ES): 567 (M + 1) for C₂₄H₁₉ClF₄N₆O₄ ¹H NMR (300 MHz, DMSO-d₆) δ ppm 1.19 (t, J = 7.16 Hz, 3 H) 3.55- 3.76 (m, 2 H) 3.89-4.27 (m, 4 H) 4.92 (t, J = 5.56 Hz, 1 H) 7.07 (d, J = 2.45 Hz, 1 H) 7.41 (t, J = 9.14 Hz, 1 H) 7.52-7.81 (m, 2 H) 7.91-8.16 (m, 2 H) 8.54 (d, J = 1.88 Hz, 1 H) 8.72 (s, 1 H) 10.37 (s, 1 H) | 5-Bromo-N-(3- chloro-4- fluorophenyl)-4- [3-(trifluoro- methyl)-1H- pyrazol-1- yl]pyrimidin-2- amine Intermediate 115 and ethyl 1-(2- hydroxyethyl)-2- oxo-5-(4,4,5,5- tetramethyl-1,3,2- dioxaborolan-2- yl)-1,2- dihydropyridine- 3-carboxylate Intermediate 430 |
| Example 1016 | 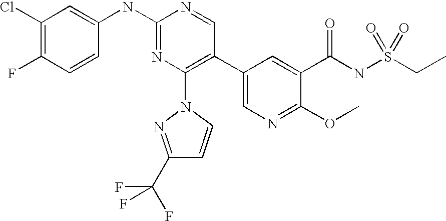 5-(2-(3-Chloro-4- fluorophenylamino)-4-(3- (trifluoromethyl)-1H- pyrazol-1-yl)pyrimidin-5- yl)-N-(ethylsulfonyl)-2- methoxynicotinamide | MS(ES): 600 (M + 1) for C₂₃H₁₈ClF₄N₇O₄S ¹H NMR (300 MHz, DMSO-d₆) δ ppm 1.26 (t, J = 7.35 Hz, 3 H) 3.36- 3.57 (m, 2 H) 3.96 (s, 3 H) 7.04 (d, J = 2.64 Hz, 1 H) 7.42 (t, J = 9.14 Hz, 1 H) 7.61-7.87 (m, 2 H) 8.09 (dd, J = 6.78, 2.64 Hz, 1 H) 8.19 (d, J = 2.07 Hz, 1 H) 8.47 (s, 1 H) 8.79 (s, 1 H) 10.41 (s,1 H) 11.65 (s,1 H) | 5-Bromo-N-(3- chloro-4- fluorophenyl)-4- [3-(trifluoro- methyl)-1H- pyrazol-1- yl]pyrimidin-2- amine Intermediate 115 and N-(ethylsulfonyl)- 2-methoxy-5- (4,4,5,5- tetramethyl-1,3,2- dioxaborolan-2- yl)nicotinamide Intermediate 431 |
| Example 1017 | 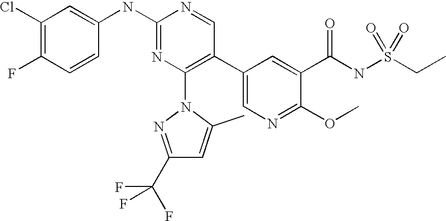 5-(2-(3-Chloro-4- fluorophenylamino)-4-(5- methyl-3-(trifluoromethyl)- 1H-pyrazol-1- yl)pyrimidin-5-yl)-N- (ethylsulfonyl)-2- methoxynicotinamide | MS(ES): 614 (M + 1) for C₂₄H₂₀ClF₄N₇O₄S ¹H NMR (300 MHz, DMSO-d₆) δ ppm 1.26 (t, 3 H) 2.33 (s, 3 H) 3.36- 3.55 (m, 2 H) 3.93 (s, 3 H) 6.76 (s, 1 H) 7.42 (t, J = 9 9.14 Hz, 1 H) 7.53- 7.77 (m, 2 H) 7.93-8.21 (m, 2 H) 8.96 (s, 1 H) 10.44 (s, 1 H) 11.67 (s, 1 H) | 5-Bromo-N-(3- chloro-4- fluorophenyl)-4- [5-methyl-3- (trifluoromethyl)- 1H-pyrazol-1- yl]pyrimidin-2- amine Intermediate 113 and N-(ethylsulfonyl)- 2-methoxy-5- (4,4,5,5- tetramethyl-1,3,2- dioxaborolan-2- yl)nicotinamide Intermediate 431 |
| Example 1018 | 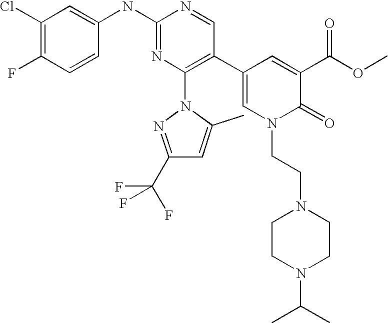 Methyl 5-(2-(3-chloro-4- fluorophenylamino)-4-(5- methyl-3-(trifluoromethyl)- 1H-pyrazol-1- yl)pyrimidin-5-yl)-1-(2-(4- isopropylpiperazin-1- yl)ethyl)-2-oxo-1,2- dihydropyridine-3- carboxylate | MS(ES): 677 (M + 1) for C₃₁H₃₃ClF₄N₈O₃ ¹H NMR (300 MHz, DMSO-d₆) δ ppm 0.94 (d, J = 6.40 Hz, 6 H) 2.25- 2.47 (m, 14 H) 3.65 (s, 3 H) 4.02 (t, J = 6.12 Hz, 2 H) 6.80 (s, 1 H) 7.26 (d, J = 2.64 Hz, 1 H) 7.41 (t, J = 9.04 Hz, 1 H) 7.63 (ddd, J = 9.09, 4.19, 2.73 Hz, 1 H) 8.01-8.19 (m, 2 H) 8.87 (s, 1 H) 10.41 (s, 1 H) | 5-Bromo-N-(3- chloro-4- fluorophenyl)-4- [5-methyl-3- (trifluoromethyl)- 1H-pyrazol-1- yl]pyrimidin-2- amine Intermediate 113 and methyl 1-(2-(4- isopropylpiperazin- 1-yl)ethyl)-2- oxo-5-(4,4,5,5- tetramethyl-1,3,2- dioxaborolan-2- yl)-1,2- dihydropyridine- 3-carboxylate Intermediate 433 |
| Example 1019 | 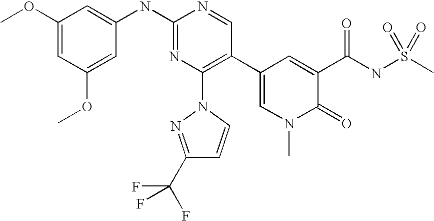 5-(2-(3,5- Dimethoxyphenylamino)- 4-(3-(trifluoromethyl)-1H- pyrazol-1-yl)pyrimidin-5- yl)-1-methyl-N- (methylsulfonyl)-2-oxo- 1,2-dihydropyridine-3- carboxamide | MS(ES): 594 (M + 1) for C₂₄H₂₂F₃N₇O₆S ¹H NMR (300 MHz, DMSO-d₆) δ ppm 3.35 (s, 3 H) 3.65 (s, 3 H) 3.75 (s, 6 H) 6.22 (t, J = 2.17 Hz, 1 H) 6.95-7.23 (m, 3 H) 8.09 (d, J = 2.64 Hz, 1 H) 8.44 (d, J= 2.83 Hz, 1 H) 8.52-8.65 (m, 1 H) 8.74 (s, 1 H) 10.17 (s, 1 H) 12.79 (s, 1 H) | 5-Bromo-N-(3,5- dimethoxyphenyl)-nl 4-(3- (trifluoromethyl)- 1H-pyrazol-1- yl)pyrimidin-2- amine Intermediate 215 and 1-methyl-N- (methylsulfonyl)- 2-oxo-5-(4,4,5,5- tetramethyl-1,3,2- dioxaborolan-2- yl)-1,2- dihydropyridine- 3-carboxamide Intermediate 432 |
| Example 1020 | 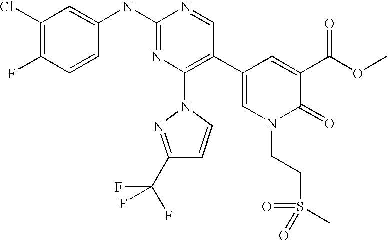 Methyl 5-(2-(3-chloro-4- fluorophenylamino)-4-(3- (trifluoromethyl)-1H- pyrazol-1-yl)pyrimidin-5- yl)-1-(2- (methylsufonyl)ethyl)-2- oxo-1,2-dihydropyridine-3- carboxylate | MS(ES): 615 (M + 1) for C₂₄H₁₉ClF₄N₆O₅S ¹H NMR (300 MHz, DMSO-d₆) δ ppm 3.06 (s, 3 H) 3.61 (t, J = 6.88 Hz, 2 H) 3.68 (s, 3 H) 4.38 (t, J = 6.88 Hz, 2 H) 7.09 (d, J = 2.64 Hz, 1 H) 7.42(t, J = 9.14 Hz, 1 H) 7.60-7.79 (m, 2 H) 8.06 (dd, J = 6.69, 2.54 Hz, 1 H) 8.20 (d, J = 2.83 Hz, 1 H) 8.57 (d, J = 2.07 Hz, 1 H) 8.72 (s, 1 H) 10.38 (s, 1 H) | 5-Bromo-N-(3- chloro-4- fluorophenyl)-4- [3-(trifluoro- methyl)-1H- pyrazol-1- yl]pyrimidin-2- amine Intermediate 115 and methyl 1-(2- (methylsulfonyl)- ethyl)-2-oxo-5- (4,4,5,5- tetramethyl-1,3,2- dioxaborolan-2- yl)-1,2- dihydropyridine- 3-carboxylate Intermediate 434 |
| Example 1021 | 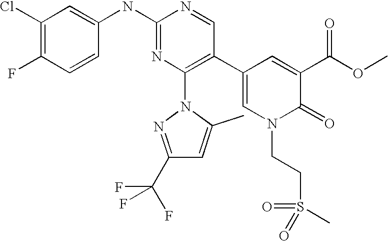 Methyl 5-(2-(3-chloro-4- fluorophenylamino)-4-(5- methyl-3-(trifluoromethyl)- 1H-pyrazol-1- yl)pyrimidin-5-yl)-1-(2- (methylsulfonyl)ethyl)-2- oxo-1,2-dihydropyridine-3- carboxylate | MS(ES): 629 (M + 1) for C₂₅H₂₁ClF₄N₆O₅S ¹H NMR (300 MHz, DMSO-d₆) δ ppm 2.36 (s, 3 H) 3.07 (s, 3 H) 3.59 (t, J = 6.78 Hz, 2 H) 3.65 (s, 3 H) 4.37 (t, J = 6.78 Hz, 2 H) 6.80 (s, 1 H) 7.22 (d, J = 2.64 Hz, 1 H) 7.41 (t, J = 9.04 Hz, 1 H) 7.64 (ddd, J = 9.04, 4.24, 2.73 Hz, 1 H) 8.05 (dd, J = 6.69, 2.54 Hz, 1 H) 8.26 (d, J = 2.83 Hz, 1 H) 8.87 (s, 1 H) 10.42 (s, 1 H) | 5-Bromo-N-(3- chloro-4- fluorophenyl)-4- [5-methyl-3- (trifluoromethyl)- 1H-pyrazol-1- yl]pyrimidin-2- amine Intermediate 113 and methyl 1-(2- (methylsulfonyl)- ethyl)-2-oxo-5- (4,4,5,5-tetramethyl-1,3,2- dioxaborolan-2- yl)-1,2- dihydropyridine- 3-carboxylate Intermediate 434 |
| Example 1022 | 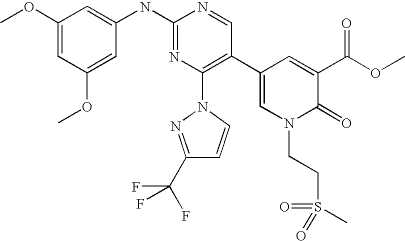 Methyl 5-(2-(3,5- dimethoxyphenylamino)-4- (3-(trifluoromethyl)-1H- pyrazol-1-yl)pyrimidin-5- yl)-1-(2- (methylsulfonyl)ethyl)-2- oxo-1,2-dihydropyridine-3- carboxylate | MS(ES): 623 (M + 1) for C₂₆H₂₅F₃N₆O₇S ¹H NMR (300 MHz, DMSO-d₆) δ ppm 3.06 (s,3 H) 3.61 (t, J = 6.88 Hz, 2 H) 3.68 (s, 3 H) 3.74 (s, 6 H) 4.38 (t, J = 6.78 Hz, 2 H) 6.21 (t, J = 2.26 Hz, 1H) 7.08 (d, J = 2.26 Hz, 3 H) 7.66 (d, J = 2.64 Hz, 1 H) 8.21 (d, J = 2.64 Hz, 1 H) 8.54 (dd, J = 2.64, 0.75 Hz, 1 H) 8.69 (s, 1 H) 10.14 (s, 1 H) | 5-Bromo-N-(3,5- dimethoxyphenyl)- 4-(3- (trifluoromethyl)- 1H-pyrazol-1- yl)pyrimidin-2- amine Intermediate 215 and methyl 1-(2- (methylsulfonyl)- ethyl)-2-oxo-5- (4,4,5,5- tetramethyl-1,3,2- dioxaborolan-2- yl)-1,2- dihydropyridine- 3-carboxylate Intermediate 434 |
| Example 1023 | 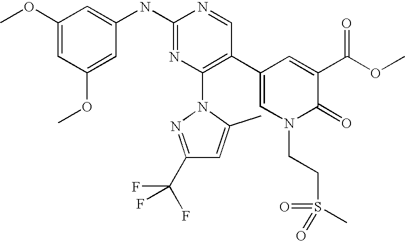 Methyl 5-(2-(3,5- dimethoxyphenylamino)-4- (5-methyl-3- (trifluoromethyl)-1H- pyrazol-1-yl)pyrimidin-5- yl)-1-(2- (methylsulfonyl)ethyl)-2- oxo-1,2-dihydropyridine-3- carboxylate | MS(ES): 637 (M + 1H) for C₂₇H₂₇F₃N₆O₇S ¹H NMR (300 MHz, DMSO-d₆) δ ppm 2.33 (s, 3 H) 3.07 (s, 3 H) 3.59 (t, J = 6.69 Hz, 2 H) 3.65 (s, 3 H) 3.72 (s, 6 H) 4.37 (t, J = 6.97 Hz, 2 H) 6.21 (t, J = 2.17 Hz, 1 H) 6.78 (s, 1 H) 7.03 (d, J = 2.26 Hz, 2 H) 7.22 (d, J = 2.83 Hz, 1 H) 8.26 (d, J = 2.83 Hz, 1 H) 8.85 (s, 1 H) 10.18 (s, 1 H) | 5-Bromo-N-(3,5- dimethoxyphenyl)- 4-(5-methyl-3- (trifluoromethyl)- 1H-pyrazol-1- yl)pyrimidin-2- amine Intermediate 216 and methyl 1-(2- (methylsulfonyl)-= ethyl)-2-oxo-5- (4,4,5,5- tetramethyl-1,3,2- dioxaborolan-2- yl)-1,2- dihydropyridine- 3-carboxylate Intermediate 434 |
| Example 1024 | 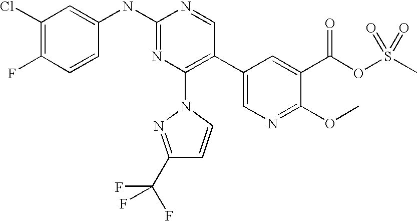 5-(2-(3-Chloro-4- fluorophenylamino)-4-(3- (trifluoromethyl)-1H- pyrazol-1-yl)pyrimidin-5- yl)-2-methoxy-N- (methylsulfonyl)- nicotinamide | MS(ES): 586 (M + 1) for C₂₂H₁₆ClF₄N₇O₄S ¹H NMR (300 MHz, DMSO-d₆) δ ppm 3.31 (s, 3 H) 3.97 (s, 3 H) 7.04 (d, J = 2.64 Hz, 1 H) 7.42 (t, J = 9.14 Hz, 1 H) 7.72 (dd, J = 4.80, 2.73 Hz, 1 H) 7.85 (d, J = 2.45 Hz, 1 H) 8.09 (dd, J = 6.78, 2.64 Hz, 1 H) 8.17 (d, J = 2.64 Hz, 1 H) 8.47 (s, 1 H) 8.79 (s, 1 H) 10.41 (s,1 H) 11.68 (s, 1 H) | 5-Bromo-N-(3- chloro-4- fluorophenyl)-4- [3-(trifluoro- methyl)-1H- pyrazol-1- yl]pyrimidin-2- amine Intermediate 115 and 2-methoxy-N- (methylsulfonyl)- 5-(4,4,5,5- tetramethyl-1,3,2- dioxaborolan-2- yl)nicotinamide Intermediate 368 |
| Example 1025 | 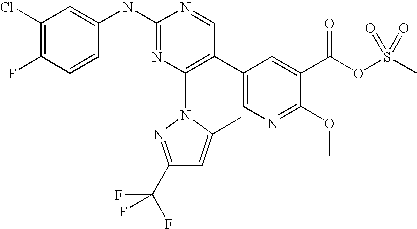 5-(2-(3-Chloro-4- fluorophenylamino)-4-(5- methyl-3-(trifluoromethyl)- 1H-pyrazol-1- yl)pyrimidin-5-yl)-2- methoxy-N- (methylsulfonyl)- nicotinamide | MS(ES): 600 (M + 1) for C₂₃H₁₈ClF₄N₇O₄S ¹H NMR (300 MHz, DMSO-d₆) δ ppm 2.34 (s,3 H) 3.32 (s, 3 H) 3.94 (s, 3 H) 6.78 (s, 1 H) 7.42 (t, J = 9.14 Hz, 1 H) 7.55- 7.77 (m, 2 H) 7.89-8.21 (m, 2 H) 8.96 (s, 1 H) 10.44 (s, 1 H) 11.71 (s, 1 H) | 5-Bromo-N-(3- chloro-4- fluorophenyl)-4- [5-methyl-3- (trifluoromethyl)- 1H-pyrazol-1- yl]pyrimidin-2- amine Intermediate 113 and 2-methoxy-N- (methylsulfonyl)- 5-(4,4,5,5- tetramethyl-1,3,2- dioxaborolan-2- yl)nicotinamide Intermediate 368 |
| Example 1026 | 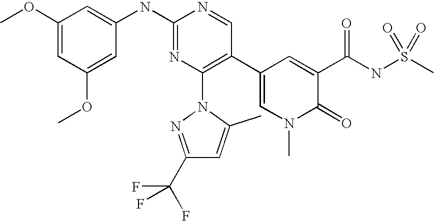 5-(2-(3,5- Dimethoxyphenylamino)- 4-(5-methoxy-3- (trifluoromethyl)-1H- pyrazol-1-yl)pyrimidin-5- yl)-1-methyl-N- (methylsulfonyl)-2-oxo- 1,2-dihydropyridine-3- carboxamide | MS(ES): 608 (M + 1) for C₂₅H₂₄F₃N₇O₆S ¹H NMR (300 MHz, DMSO-d₆) δ ppm 2.46 (br. s., 3 H) 3.28 (s, 3 H) 3.62 (br. s., 3 H) 3.73 (s, 6 H) 6.22 (t, J = 2.17 Hz, 1 H) 6.80 (s, 1 H) 7.03 (d, J= 2.07 Hz,2 H) 7.72 (br. s., 1 H) 8.40 (br. s., 1 H) 8.87 (s, 1 H_{) 10.17 (s, 1 H)} _{12.68 (s, 1 H)} | 5-Bromo-N-(3,5- dimethoxyphenyl)- 4-(5-methyl-3- (trifluoromethyl)- 1H-pyrazol-1- yl)pyrimidin-2- amine Intermediate 216 and 1-methyl-N- (methylsulfonyl)- 2-oxo-5-(4,4,5,5- tetramethyl-1,3,2- dioxaborolan-2- yl)-1,2- dihydropyridine- 3-carboxamide Intermediate 432 |

### Example 1027

### (5-(2-(3-chloro-4-fluorophenylamino)-4-(3-(trifluoromethyl)-1H-pyrazol-1-yl)pryimidin-5-yl)-2-methoxypyridin-3yl)methanol

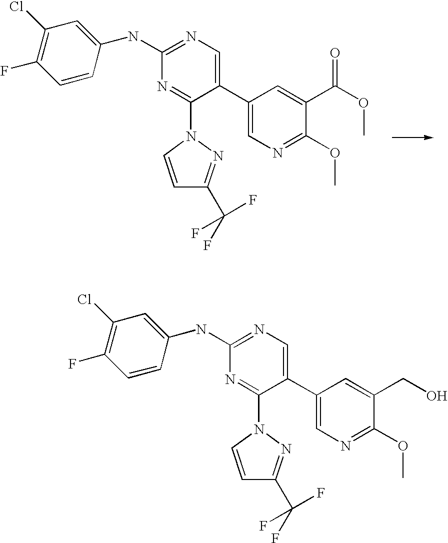

Methyl 5-(2-(3-chloro-4-fluorophenylamino)-4-(3-(trifluoromethyl)-1H-pyrazol-1-yl)pyrimidin-5-yl)-2-methoxynicotinate (Example 1010, 0.788 g mg, 1.51 mmol) was dissolved in THF(10 mL) to give a yellow solution. The reaction mixture was cooled to −40° C. 1M DIBAL-H in toluene (9 mL, 9 mmol) was slowly added to the reaction mixture. The reaction was allowed to warm up to room temperature overnight. The reaction was diluted with EtOAc and washed with 1M NH₄Cl. Purification by flash chromatography, silica gel, 40-100% ethyl acetate in hexanes gave a crude solid. Trituration with hexane/ether and filtration gave the title compound (103 mg).

MS (Electrospray): 495.83, (MH⁺) for C₂₁H₁₅ClF₄N₆O₂

¹H NMR (300 MHz, DMSO-d₆) δ: 3.88 (s, 3H) 5.15 (s, 2H) 7.00 (d, J=2.45 Hz, 1H) 7.20-7.47 (m, 2H) 7.59-7.83 (m, 1H) 7.90 (d, J=2.26 Hz, 1H) 7.99-8.23 (m, 1H) 8.38 (d, J=1.51 Hz, 1H) 8.76 (s, 1H), 10.39 (s, 1H)

### Example 1028

### 5-(2-(3-chloro-4-fluorophenylamino)-4-(3-(trifluoromethyl)-1H-pyrazol-1-yl)pryrimidin-5-yl)-2-methoxynicotinaldehyde

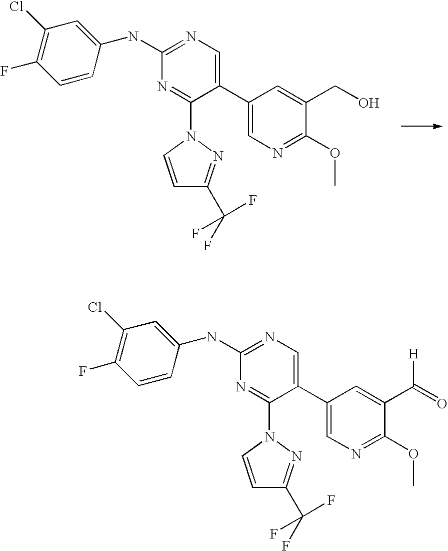

(5-(2-(3-chloro-4-fluorophenylamino)-4-(3-(trifluoromethyl)-1H-pyrazol-1-yl)pyrimidin-5-yl)-2-methoxypyridin-3-yl)methanol (243 mg, 0.49 mmol) Example 1027 and manganese dioxide (427 mg, 4.91 mmol) were combined in dichloromethane to give a black suspension. The reaction mixture was allowed to stir at room temperature for 6 hours. Additional manganese dioxide (427 mg, 4.91 mmol) was added and the mixture was allowed to stir for 48 hours. The mixture was filtered through celite and washed with methanol and dichloromethane. The filtrate was concentrated to yield the title compound (145 mg). MS (Electrospray): 493.81 (MH⁺) for C₂₁H₁₃ClF₄N₆O₂

### Example 1029

### (E)-methyl 3-(5-(2-(3-chloro-4-fluorophenylamino)-4-(3-(trifluoromethyl)-1H-pyrazol-1yl)pyrimidin-5-yl)-2-methoxypyridin-3-yl)acrylate

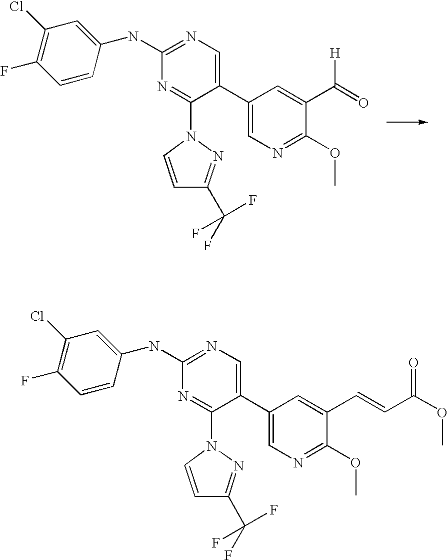

Methyl 2-(diethoxyphosphoryl)acetate (68.0 mg, 0.32 mmol) and NaH (17.65 mg, 0.44 mmol) were combined in THF (2 ml) to give a colorless solution. The reaction mixture was allowed to stir for 5 minutes then added to a solution of 5-(2-(3-chloro-4-fluorophenylamino)-4-(3-(trifluoromethyl)-1H-pyrazol-1-yl)pyrimidin-5-yl)-2-methoxynicotinaldehyde (145 mg, 0.29 mmol) Example 1028 in THF (2 ml). The mixture was allowed to stir at RT for 45 min, water and ethyl acetate were added. The organic layer was then dried with MgSO4 and concentrated. The solid was purified by flash chromatography over silica gel. The product was eluted using 30% ethyl acetate in hexanes to give the title compound (114 mg). MS (Electrospray): 548.88 (MH⁺) for C24H17ClF4N6O3

### Example 1030

### (E)-3-(5-(2-(3-chloro-4-fluorophenylamino)-4-(3-(trifluoromethyl)-1H-pyrazol-1-yl)pyrimidin-5-yl)-2-methoxypyridin-3-yl)acrylic acid

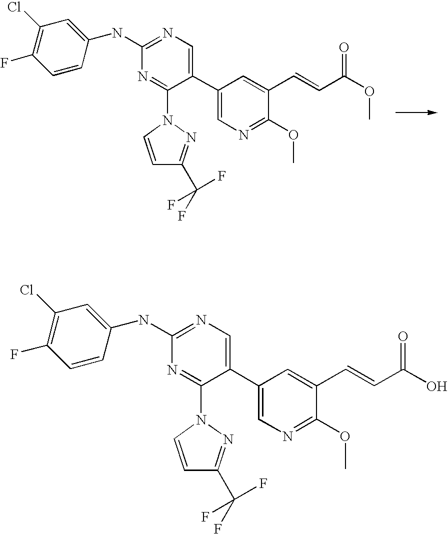

(E)-methyl 3-(5-(2-(3-chloro-4-fluorophenylamino)-4-(3-(trifluoromethyl)-1H-pyrazol-1-pyrazol-1yl)pyrimidin-5-yl)-2-methoxypyridin-3-yl)acrylate Example 1029 (114 mg, 0.21 mmol) was dissolved in Dioxane (5 mL) to give a yellow solution. 1M NaOH (0.312 mL, 0.31 mmol) was added at room temp and then allowed to stir overnight. The reaction mixture was acidified with 1M HCl then extracted with ethyl acetate. The ethyl acetate was evaporated and the solid purified using reverse phase chromatography (C18, 20 to 95%.CH₃CN/H₂O/0.1% Trifluoroacetic acid) to yield the title compound (28 mg).

MS (Electrospray): 535 (MH⁺) for C23H15ClF4N6O3

¹H NMR (300 MHz, DMSO-d₆) δ: 3.92-4.02 (m, 3H) 6.49 (d, J=16.01 Hz, 1H) 7.04 (d, J=2.64 Hz, 1H) 7.42 (t, J=9.14 Hz, 1H) 7.64 (d, J=16.20 Hz, 2H) 7.68-7.77 (m, 1H) 7.91 (d, J=2.26 Hz, 1H) 8.00-8.20(m, 2H) 8.48 (s, 1H) 8.82 (s, 1H) 10.40 (s, 1H) 12.45 (br. s., 1H)

The following examples were prepared using the general method described for Example 158 using [1,1′-bis (diphenylphosphino)ferrocene]dichloropalladium(II), sodium carbonate and the starting materials (SM) indicated.

| Ex | Compound | Data | SM |
|---|---|---|---|
| Example 1031 | 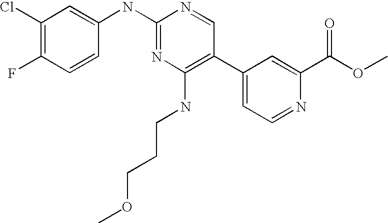 Methyl 4-(2-(3-chloro-4- fluorophenylamino)-4-(3- methoxypropylamino)- pyrimidin-5-yl)picolinate | MS(ES): 446 (M + 1) for C₂₁H₂₁ClFN₅O₃ ¹H NMR (300 MHz, DMSO-d₆) δ ppm 1.74- 1.97 (m, 2 H) 3.21 (s, 3 H) 3.37-3.54 (m, 4 H) 3.90 (s, 3 H) 7.07 (t, J = 4.90 Hz, 1 H) 7.31 (t, J = 9.14 Hz, 1 H) 7.52- 7.75 (m, 2 H) 7.94 (s, 1 H) 8.05 (d, J = 0.94 Hz, 1 | |
| H) 8.24 (dd, J = 6.97, 2.45 Hz, 1 H) 8.73 (d, J = 5.09 Hz, 1 H) 9.56 (s, 1 H) | 5-Bromo-N²-(3- chloro-4- fluorophenyl)-N⁴- (3- methoxypropyl) pyrimidine-2,4- diamine Intermediate 119 and methyl 4-(4,4,5,5- tetramethyl-1,3,2- dioxaborolan-2- yl)picolinate | | |
| Example 1032 | 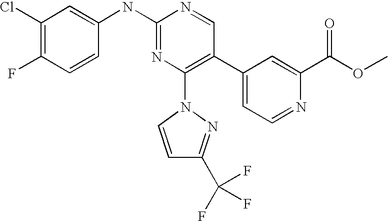 Methyl4-(2-(3-chloro-4- fluorophenylamino)-4-(3- (trifluoromethyl)-1H- pyrazol-1-yl)pyrimidin-5- yl)picolinate | MS(ES): 493 (M + 1) for C₂₁H₁₃ClF₄N₆O₂ ¹H NMR (300 MHz, DMSO-d₆) δ ppm 3.85 (s, 3 H) 7.08 (d, J = 2.64 Hz, 1 H) 7.32-7.59 (m, 2 H) 7.65-7.82 (m, 2 H) 8.07 (dd, J = 6.69, 2.54 Hz, 1 H) 8.55 (d, J = 1.70 Hz, 1 H) 8.68 (d, J = 4.90 | |
| Hz, 1 H) 8.85 (s, 1 H) 10.54 (s, 1 H) | 5-bromo-N-(3- chloro-4- fluorphenyl)-4- [3-(trifluoro- methyl)-1H- pyrazol-1- yl]pyrimidin-2- amine Intermediate 115 and methyl 4-(4,4,5,5- tetramethyl-1,3,2- dioxaborolan-2- yl)picolinate | | |
| Example 1033 | 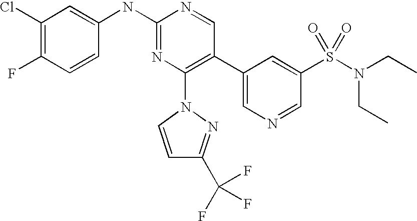 5-(2-(3-Chloro-4- fluorophenylamino)-4-(3- (trifluoromethyl)-1H- pyrazol-1-yl)pyrimidin-5- yl)-N,N-diethylpyridine-3- sulfonamide | MS(ES): 570 (M + 1) for C₂₃H₂₀ClF₄N₇O₂S ¹H NMR (300 MHz, DMSO-d₆) δ ppm 1.04 (t, 6 H) 3.14 (q, J = 7.16 Hz, 4 H) 7.05 (d, J = 2.83 Hz, 1 H) 7.44 (t, J = 9.14 Hz, 1 H) 7.74 (ddd, J = 9.00, 4.10, 2.73 Hz, 1 H) 7.95- 8.18 (m, 2H) 8.57 (d, J = 1.70 Hz, 1 H) 8.70 (d, J = 2.07 Hz, 1 H) 8.81 (s, 1 H) 8.90 (d, J = 2.07 Hz, 1 H) 10.49 (s, 1 H) | 5-bromo-N-(3- chloro-4- fluorophenyl)-4- [3-(trifluoro- methyl)-1H- pyrazol-1- yl]pyrimidin-2- amine Intermediate 115 and N,N-diethyl-5- (4,4,5,5- tetramethyl-1,3,2- dioxaborolan-2- yl)pyridine-3- sulfonamide Intermediate 412 |
| Example 1034 | 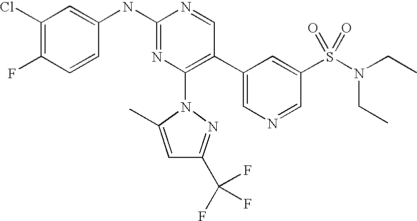 4-(2-(4-chloro-4- fluorophenylamino)-4-(5- methyl-3-(trifluoromethyl)- 1H-pyrazol-1- yl)pyrimidin-5-yl)-N,N- diethylpyridine-3- sulfonamide | MS(ES): 584 (M + 1) for C₂₄H₂₂ClF₄N₇O₂S ¹H NMR (300 MHz, DMSO-d₆) δ ppm 1.03 (t, 6 H) 2.50 (s, 3H) 3.11 (q, J = 7.10 Hz, 4 H) 6.79 (s, 1 H) 7.44 (t, J = 9.14 Hz, 1 H) 7.56-7.75 (m, 1 H) | |
| 7.87 (t, J = 1.88 Hz, 1 H) 8.07 (dd, J = 6.59, 2.26 Hz, 1 H) 8.59 (d, J = 1.88 Hz, 1 H) 8.87 (d, J = 2.07 Hz, 1 H) 8.96 (s, 1 H) 10.50 (s, 1 H) | 5-bromo-N-(3- chloro-4- fluorophenyl)-4- [5-methyl-3- (trifluoromethyl)- 1H-pyrazol-1- yl]pyrimidin-2- amine Intermediate 113 and N,N-diethyl-5- (4,4,5,5- tetramethyl-1,3,2- dioxaborolan-2- yl)pyridine-3- sulfonamide Intermediate 412 | | |
| Example 1035 | 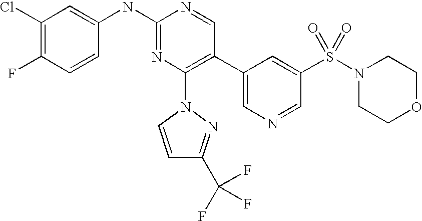 N-(3-chloro-4- fluorophenyl)-5-(5- (morpholinosulfonyl)pyrimidin-3-yl)-4-(3- (trifluoromethyl)-1H- pyrazol-1-yl)pyrimidin-2- amine | MS(ES): (M + 1) for C₂₃H₁₈ClF₄N₇O₃S ¹H NMR (300 MHz, DMSO-d₆) δ ppm 2.79- 3.00 (m, 4 H) 3.49-3.76 (m, 4 H) 7.06 (d, J = 2.64 Hz, 1 H) 7.44 (t, J = 9.14 Hz, 1 H) 7.63-7.83 (m, 1 H) 7.96-8.15 (m, 2 H) | |
| 8.59 (d, J = 1.70 Hz, 1 H) 8.77 (d, J = 2.07 Hz, 1 H) 8.80-8.91 (m, 2 H) 10.49 (s, 1 H) | 5-bromo-N-(3- chloro-4- fluorophenyl)-4- [3-(trifluoro- methyl)-1H- pyrazol-1- yl]pyrimidin-2- amine Intermediate 115 and 4-(5-(4,4,5,5- tetramethyl-1,3,2- dioxaborolan-2- yl)pyridin-3- ylsulfonyl) morpholine Intermediate 413 | | |
| Example 1036 | 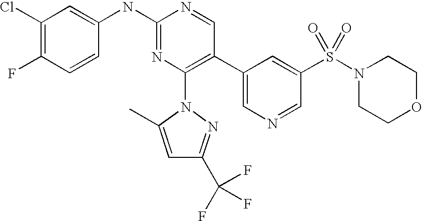 N-(3-chloro-4- fluorophenyl)-4-(5-methyl- 3-(trifluoromethyl)-1H- pyrazol-1-yl)-5-(5- (morpholinosulfonyl)pyrimidin-3-yl)pyrimidin- 2-amine | MS(ES): 598 (M + 1) for C₂₄H₂₀ClF₄N₇O₃S ¹H NMR (300 MHz, DMSO-d₆) δ ppm 2.54 (s, 3 H) 2.77-2.96 (m, 4 H) 3.52-3.77 (m, 4 H) 6.79 (s,1 H) 7.44 (t, J = 9.14 Hz, 1 H) 7.66 (ddd, J = 8.95, 4.24, 2.83 | |
| Hz, 1 H) 7.86 (t, J = 2.17 Hz, 1 H) 7.98-8.18 (m, 1 H) 8.67 (d, J = 2.07 Hz, 1 H) 8.82 (d, J = 2.07 Hz, 1 H) 8.98 (s, 1 H) 10.50 (s, 1 H) | 5-bromo-N-(3- chloro-4- fluorophenyl)-4- [5-methyl-3- (trifluoromethyl)- 1H-pyrazol-1- yl]pyrimidin-2- amine Intermediate 113 and 4-(5-(4,4,5,5- tetramethyl-1,3,2- dioxaborolan-2- yl)pyridin-3- ylsulfonyl) morpholine Intermediate 413 | | |
| Example 1037 | 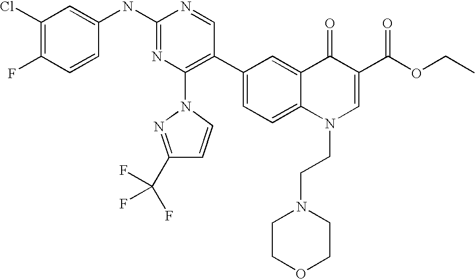 Ethyl 6-(2-(3-chloro-4- fluorophenylamino)-4-(3- (trifluoromethyl)-1H- pyrazol-1-yl)pyrimidin-5- yl)-1-(2-morpholinoethyl)- 4-oxo-1,4- dihydroquinoline-3- carboxylate | MS(ES): 686 (M + 1) for C₃₂H₂₈ClF₄N₇O₄ ¹H NMR (300 MHz, DMSO-d₆) δ ppm 1.29 (t, J = 7.06 Hz, 3 H) 2.33- 2.45 (m, 4 H) 2.60 (t, J = 5.46 Hz, 2 H) 3.43- 3.61 (m, 4 H) 4.23 (q, | |
| J = 7.03 Hz, 2 H) 4.49 (t, J = 4.99 Hz, 2 H) 7.00 (d, J = 2.64 Hz, 1 H) 7.32- 7.59 (m, 2 H) 7.67-7.89 (m, 2 H) 8.05 (d, J = 2.26 Hz, 1 H) 8.12 (dd, J = 6.78, 2.64 Hz, 1 H) 8.44 (d, J = 1.51 Hz, 1 H) 8.62 (s, 1 H) 8.84 (s, 1 H) 10.44 (s, 1 H) | 5-bromo-N-(3- chloro-4- fluorophenyl)-4- [3-(trifluoro- methyl)-1H- pyrazol-1- yl]pyrimidin-2- amine Intermediate 115 and Ethyl 1-(2- methoxyethyl)-4- oxo-6-(4,4,5,5- tetramethyl-1,3,2- dioxoborolan-2- yl)-1,4- dihydroquinoline- 3-carboxylate Intermediate 134 | | |
| Example 1038 | 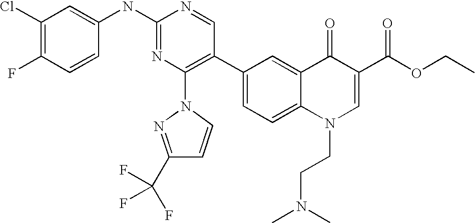 Ethyl 6-(2-(3-chloro-4- fluorophenylamino)-4-(3- (trifluoromethyl)-1H- pyrazol-1-yl)pyrimidin-5- yl)-1-(2- (dimethylamino)ethyl)-4- oxo-1,4-dihydroquinoline- 3-carboxylate | MS(ES): 644 (M + 1) for C₃₀H₂₆ClF₄N₇O₃ ¹H NMR (300 MHz, DMSO-d₆) δ ppm 1.29 (t, J = 7.06 Hz, 3 H) 2.18 (s, 6 H) 2.57 (t, J = 5.56 Hz, 2 H) 4.23 (q, J = 7.03 Hz, 2 | |
| H) 4.47 (t, J = 5.09 Hz, 2 H) 7.00 (d, J = 2.64 Hz, 1 H) 7.32-7.62 (m, 2 H) 7.63-7.89 (m, 2 H) 7.98- 8.19 (m, 2 H) 8.45 (d, J = 1.88 Hz, 1 H) 8.61 (s, 1 H) 8.84 (s, 1 H) 10.44 (s, 1 H) | 5-bromo-N-(3- chloro-4- fluorophenyl)-4- [3-(trifluoro- methyl)-1H- pyrazol-1- yl]pyrimidin-2- amine Intermediate 115 and ethyl 1-(2- (dimethylamino) ethyl)-4-oxo-6- (4,4,5,5- tetramethyl-1,3,2- dioxaborolan-2- | | |
| yl)-1,4- dihydroquinoline- 3-carboxylate Intermediate 414 | | | |
| Example 1039 | 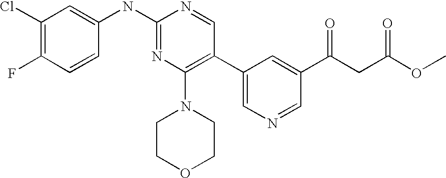 Methyl 3-(5-(2-(3-chloro- 4-fluorophenylamino)-4- morpholinopyrimidin-5- yl)pyridin-3-yl)-3- oxopropanoate | MS(ES): 486 (M + 1) for C₂₃H₂₁ClFN₅O₄ ¹H NMR (300 MHz, DMSO-d₆) δ ppm 3.09- 3.42 (m, 4 H) 3.46-3.61 (m, 4 H) 3.68 (s, 3 H) 4.34 (s, 2 H) 7.38 (t, J = 9.14 Hz, 1 H) 7.59 (ddd, J = 9.09, 4.19, 2.73 Hz, 1 H) 8.07 (dd, J = 6.88, 2.54 Hz, 1 H) | |
| 8.18 (s, 1 H) 8.38 (t, J = 2.17 Hz, 1 H) 8.94 (d, J = 2.26 Hz, 1 H) 9.07 (d, J = 2.07 Hz, 1 H) 10.01 (s, 1 H) | 5-Bromo-N-(3- chloro-4- fluorophenyl)-4- morpholin-4- ylpyrimidin-2- amine Intermediate 111 and methyl 3-oxo-3- (5-(4,4,5,5- tetramethyl-1,3,2- dioxaborolan-2- yl)pyridin-3- yl)propanoate Intermediate 417 | | |
| Example 1040 | 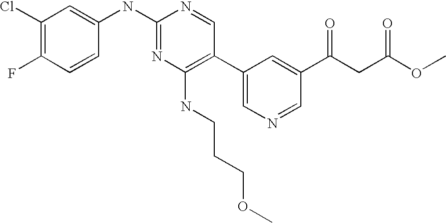 Methyl 3-(5-(2-(3-chloro- 4-fluorophenylamino)-4- (3- methoxypropylamino)pyrimidin-5-yl)pyridin-3-yl)-3- oxopropanoate | MS(ES): 488 (M + 1) for C₂₃H₂₃ClFN₅O₄ ¹H NMR (300 MHz, DMSO-d₆) δ ppm 1.64- 1.94 (m, 2 H) 3.19 (s, 3 H) 3.27-3.55 (m, 4 H) 3.68 (s, 3 H) 4.35 (s, 2 H) 7.31-7.47 (m, 1 H) 7.57 (ddd, J = 9.00, 4.19, 2.64 Hz, 1 H) 7.71 (br. s., 1 H) | |
| 7.85-7.98 (m, 1 H) 8.09 (dd, J = 6.78, 2.45 Hz, 1 H) 8.30 (t, J = 2.17 Hz, 1 H) 8.83 (d, J = 2.07 Hz, 1 H) 9.14 (d, J = 2.07 Hz, 1 H) 9.99 (br. s., 1 H) | 5-Bromo-N²-(3- chloro-4- fluorophenyl)-N⁴- (3- methoxypropyl) pyrimidin-2,4- diamine Intermediate 119 and methyl 3-oxo-3- (5-(4,4,5,5- tetramethyl-1,3,2- dioxaborolan-2- yl)pyridin-3- yl)propanoate Intermediate 417 | | |
| Example 1041 | 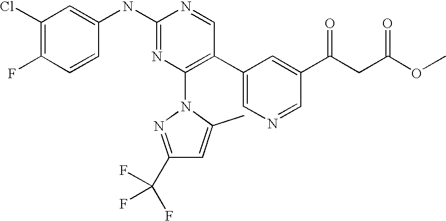 Methyl 3-(5-(2-(3-chloro- 4-fluorophenylamino)-4- (5-methyl-3- (trifluoromethyl)-1H- pyrazol-1-yl)pyrimidin-5- yl)pyridin-3-yl)-3- oxopropanoate | MS(ES): 549 (M + 1) for C₂₄H₁₇ClF₄N₆O₃ ¹H NMR (300 MHz, DMSO-d₆) δ ppm 2.45 (s, 3 H) 3.65 (s, 3 H) 4.24 (s, 2 H) 6.78 (s, 1 H) 7.43 (t, J = 9.14 Hz, 1 H) 7.66 (ddd, J = 8.95, 4.14, 2.73 Hz, 1 H) 7.81-8.16 (m, | |
| 2 H) 8.48 (d, J = 2.07 Hz, 1 H) 8.85-9.16 (m, 2 H) 10.48 (s, 1 H) | 5-Bromo-N-(3- chloro-4- fluorophenyl)-4- [5-methyl-3- (trifluoromerthyl)- 1H-pyrazol-1- yl]pyrimidin-2- amine Intermediate 113 and methyl 3-oxo-3- (5-(4,4,5,5- tetramethyl-1,3,2- dioxaborolan-2- yl)pyridin-3- yl)propanoate Intermediate 417 | | |
| Example 1042 | 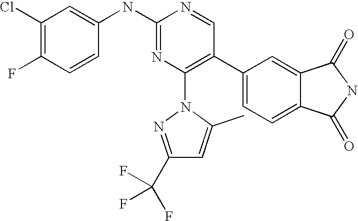 5-(2-(3-Chloro-4- fluorophenylamino)-4-(5- methyl-3-(trifluoromethyl)- 1H-pyrazol-1- yl)pyrimidin-5- yl)isoindoline-1,3-dione | MS(ES): 517 (M + 1) for C₂₃H₁₃ClF₄N₆O₂ ¹H NMR (300 MHz DMSO-d₆) δ ppm 2.41 (s, 3 H) 6.76 (s, 1 H) 7.33- 7.59 (m, 3 H) 7.67 (ddd, J = 9.09, 4.19, 2.73 Hz, 1 H) 7.77 (d, J = 7.72 Hz, 1 H) 8.07 (dd, J = 6.78, 2.45 | |
| Hz, 1 H) 8.98 (s, 1 H) 10.48 (s, 1 H) 11.36 (s, 1 H) | 5-Bromo-N-(3- chloro-4- fluorophenyl)-4- [5-methyl-3- (trifluoromethyl)- 1H-pyrazol-1- yl]pyrimidin-2- amine Intermediate 113 and 5-(4,4,5,5- tetramethyl-1,3,2- dioxaborolan-2- yl)isoindoline- 1,3-dione Intermediate 427 | | |
| Example 1043 | 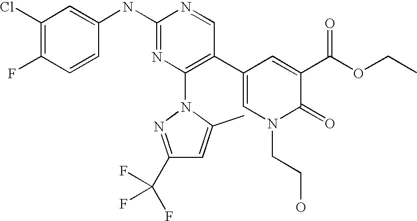 Ethyl 5-(2-(3-chloro-4- fluorophenylamino)-4-(5- methyl-3-(trifluoromethyl)- 1H-pyrazol-1- yl)pyrimidin-5-yl)-1-(2- hydroxyethyl)-2-oxo-1,2- dihydropyridine-3- carboxylate | MS(ES): 581 (M + 1) for C₂₅H₂₁ClF₄N₆O₄ ¹H NMR (300 MHz, DMSO-d₆) δ ppm 1.19 (t, J = 7.06 Hz, 3 H) 2.35 (s, 3 H) 3.54-3.70 (m, 2 H) 3.92-4.19 (m, 4 H) 4.91 (t, J = 5.18 Hz, 1 H) 6.79 | |
| (s, 1 H) 7.20-7.47 (m, 2 H) 7.57-7.73 (m, 1 H) 7.90-8.18 (m, 2 H) 8.86 (s, 1 H) 10.40 (s, 1 H) | 5-Bromo-N-(3- chloro-4- fluorophenyl)-4- [5-methyl-3- (trifluoromethyl)- 1H-pyrazol-1- yl]pyrimidin-2- amine Intermediate 113 and ethyl 1-(2- hydroxyethyl)-2- oxo-5-(4,4,5,5- tetramethyl-1,3,2- dioxaborolan-2- yl)-1,2- dihydropyridine- 3-carboxylate Intermediate 430 | | |
| Example 1044 | 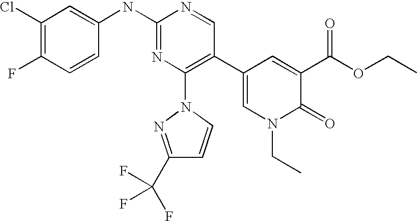 Ethyl 5-(2-(3-chloro-4- fluorophenylamino)-4-(3- (trifluoromethyl)-1H- pyrazol-1-yl)pyrimidin-5- yl)-1-ethyl-2-oxo-1,2- dihydropyridine-3- carboxylate | MS(ES): 551 (M + 1) for C₂₄H₁₉ClF₄N₆O₃ ¹H NMR (300 MHz, DMSO-d₆) δ ppm 1.18- 1.33 (m, 6 H) 3.96 (q, J = 7.03 Hz, 2 H) 4.15 (q, J = 7.16 Hz, 2 H) 7.08 (d, J = 2.64 Hz, 1 H) 7.41 (t, | |
| J = 9.14 Hz, 1 H) 7.62 (d, J = 2.64 Hz, 1 H) 7.70 (ddd, J = 9.00, 4.29, 2.73 Hz, 1 H) 7.96-8.16 (m, 2 H) 8.56 (dd, J = 2.54, 0.85 Hz, 1 H) 8.78 (s, 1 H) 10.36 (s, 1 H) | 5-Bromo-N-(3- chloro-4- fluorophenyl)-4- [3-(trifluoro- methyl)-1H- pyrazol-1- yl]pyrimidin-2- amine Intermediate 115 and ethyl 1-ethyl-2- oxo-5-(4,4,5,5- tetramethyl-1,3,2- dioxaborolan-2- yl)-1,2- dihydropyridine- 3-carboxylate Intermediate 370 | | |
| Example 1045 | 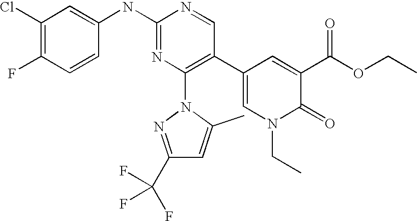 Ethyl 5-(2-(3-chloro-4- fluorophenylamino)-4-(5- methyl-3-(trifluoromethyl)- 1H-pyrazol-1- yl)pyrimidin-5-yl)-1-ethyl- 2-oxo-1,2-dihydropyridine- 3-carboxylate | MS(ES): 565 (M + 1) for C₂₅H₂₁ClF₄N₆O₃ ¹H NMR (300 MHz, DMSO-d₆) δ ppm 1.16- 1.24 (m, 6 H) 2.40 (s, 3 H) 3.92-4.28 (m, 4 H) 6.80 (s, 1 H) 7.25-7.49 (m, 2 H) 7.64 (ddd, J = 9.14, 4.24, 2.64 Hz, 1 | |
| H) 7.97-8.15 (m, 2 H) 8.92 (s, 1 H) 10.39 (s, 1 H) | 5-bromo-N-(3- chloro-4- fluorophenyl)-4- [5-methyl-3- (trifluoromethyl)- 1H-pyrazol-1- yl]pyrimidin-2- amine Intermediate 113 and ethyl 1-ethyl-2- oxo-5-(4,4,5,5- tetramethyl-1,3,2- dioxaborolan-2- yl)-1,2- dihydropyridine- 3-carboxylate Intermediate 370 | | |
| Example 1046 | 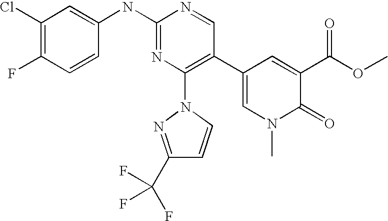 Methyl 5-(2-(3-chloro-4- fluorophenylamino)-4-(3- (trifluoromethyl)-1H- pyrazol-1-yl)pyrimidin-5- yl)-1-methyl-2-oxo-1,2- dihydropyridine-3- carboxylate | MS(ES): 523 (M + 1) for C₂₂H₁₅ClF₄N₆O₃ ¹H NMR (300 MHz, DMSO-d₆) δ ppm 3.50 (s, 3 H) 3.68 (s, 3 H) 7.08 (d, J = 2.64 Hz, 1 H) 7.41 (t, J = 9.14 Hz, 1 H) 7.57- 7.79 (m, 2 H) 8.05 (dd, | |
| J = 6.78, 2.64 Hz, 1 H) 8.16 (d, J = 2.64 Hz, 1 H) 8.57 (dd, J = 2.64, 0.94 Hz, 1 H) 8.75 (s, 1 H) 10.36 (s, 1 H) | 5-Bromo-N-(3- chloro-4- fluorophenyl)-4- [3-(trifluoro- methyl)-1H- pyrazol-1- yl]pyrimidin-2- amine Intermediate 115 and methyl 1-methyl- 2-oxo-5-(4,4,5,5- tetramethyl-1,3,2- dioxaborolan-2- yl)-1,2- dihydropyridine- 3-carboxylate Intermediate 369 | | |
| Example 1047 | 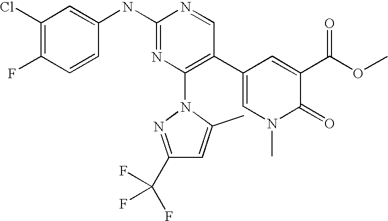 Methyl 5-(2-(3-chloro-4- fluorophenylamino)-4-(5- methyl-3-(trifluoromethyl)- 1H-pyrazol-1- yl)pyrimidin-5-yl)-1- methyl-2-oxo-1,2- dihydropyridine-3- carboxylate | MS(ES): 537 (M + 1) for C₂₃H₁₇ClF₄N₆O₃ ¹H NMR (300 MHz, DMSO-d₆) δ ppm 2.38 (s,3 H) 3.48 (s, 3 H) 3.65 (s, 3 H) 6.80 (s, 1 H) 7.27 (d, J = 2.64 Hz, 1 H) 7.41 (t, J = 9.04 Hz, 1 H) 7.64 | |
| (ddd, J = 9.04, 4.14, 2.83 Hz, 1 H) 8.04 (dd, J = 6.78, 2.64 Hz, 1 H) 8.16 (d, J = 2.64 Hz, 1H) 8.89 (s, 1 H) 10.39 (s, 1 H) | 5-Bromo-N-(3- chloro-4- fluorophenyl)-4- [5-methyl-3- (trifluoromethyl)- 1H-pyrazol-1- yl]pyrimidin-2- amine Intermediate 113 and methyl 1-methyl- 2-oxo-5-(4,4,5,5- tetramethyl-1,3,2- dioxaborolan-2- yl)-1,2- dihydropyridine- 3-carboxylate Intermediate 369 | | |
| Example 1048 | 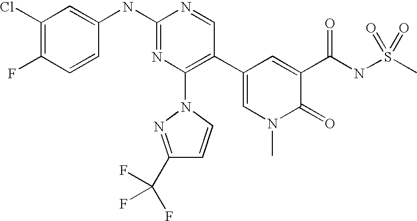 5-(2-(3-Chloro-4- fluorophenylamino)-4-(3- (trifluoromethyl)-1H- pyrazol-1-yl)pyrimidin-5- yl)-1-methyl-N- (methylsulfonyl)-2-oxo- 1,2-dihydropyridine-3- carboxamide | MS(ES): 586 (M + 1) for C₂₂H₁₆ClF₄N₇O₄S ¹H NMR (300 MHz, DMSO-d₆) δ ppm 3.35 (s, 3 H) 3.65 (s, 3 H) 7.09 (d, J = 2.64 Hz, 1 H) 7.43 (t, J = 9.14 Hz, 1 H) 7.72 (ddd, J = 9.09, 4.29, 2.64 Hz, 1 H) 7.94-8.19 (m, 2 H) 8.43 (d, J = 2.45 Hz, 1 H) 8.61 (d, J = 1.70 Hz, 1 H) 8.76 (s, 1 H) 10.40 (s, 1 H) 12.79 (s, 1 H) | 5-Bromo-N-(3- chloro-4- fluorophenyl)-4- [3-(trifluoro- methyl)-1H- pyrazol-1- yl]pyrimidin-2- amine Intermediate 115 and 1-methyl-N- (methylsulfonyl)- 2-oxo-5-(4,4,5,5- tetramethyl-1,3,2- dioxaborolan-2- yl)-1,2- dihydropyridine- 3-carboxamide |
| Intermediate 432 | | | |
| Example 1049 | 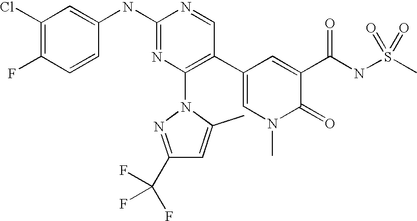 5-(2-(3-Chloro-4- fluorophenylamino)-4-(5- methyl-3-(trifluoromethyl)- 1H-pyrazol-1- yl)pyrimidin-5-yl)-1- methyl-N- (methylsulfonyl)-2-oxo- 1,2-dihydropyridine-3- carboxamide | MS(ES): 600 (M + 1) for C₂₃H₁₈ClF₄N₇O₄S ¹H NMR (300 MHz, DMSO-d₆) δ ppm 2.49 (br. s., 3 H) 3.33 (s, 3 H) 3.63 (s, 3 H) 6.82 (s, 1 H) 7.42 (t, J = 9.14 Hz, 1 H) | |
| 7.54-7.78 (m, 2 H) 8.04 (dd, J = 6.78, 2.45 Hz, 1 H) 8.40 (d, J = 2.45 Hz, 1 H) 8.90 (s, 1 H) 10.41 (s, 1 H) 12.68 (s, 1 H) | 5-Bromo-N-(3- chloro-4- fluorophenyl)-4- [5-methyl-3- (trifluoromethyl)- 1H-pyrazol-1- yl]pyrimidin-2- amine Intermediate 113 and 1-methyl-N- (methylsuldonyl)- 2-oxo-5-(4,4,5,5- tetramethyl-1,3,2- dioxaborolan-2- yl)-1,2- dihydropyridine- 3-carboxamide Intermediate 432 | | |
| Example 1050 | 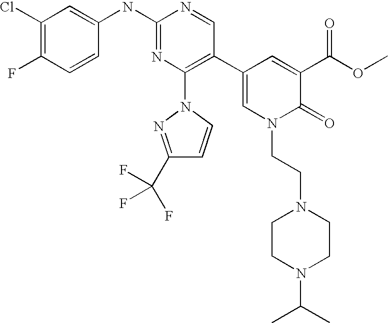 Methyl 5-(2-(3-chloro-4- fluorophenylamino)-4-(3- (trifluoromethyl)-1H- pyrazol-1-yl)pyrimidin-5- yl)-1-(2-(4- isopropylpiperazin-1- yl)ethyl)-2-oxo-1,2- dihydropyridine-3- carboxylate | MS(ES): 663 (M + 1) for C₃₀H₃₁ClF₄N₈O₃ ¹H NMR (300 MHz, DMSO-d₆) δ ppm 0.92 (d, J = 5.65 Hz, 6 H) 2.30- 2.70 (m, 14 H) 4.03 (t, J = 5.65 Hz, 2 H) 7.08 (d, | |
| J = 2.64 Hz, 1 H) 7.41 (t, J = 9.14 Hz, 1 H) 7.59- 7.78 (m, 2 H) 7.96-8.17 (m, 2 H) 8.57 (d, J = 1.70 Hz, 1 H) 8.71 (s, 1 H) 10.36 (s, 1 H) | 5-Bromo-N-(3- chloro-4- fluorophenyl)-4- [3-(trifluoro- methyl)-1H- pyrazol-1- yl]pyrimidin-2- amine Intermediate 115 and methyl 1-(2-(4- isopropylpiperazin- 1-yl)ethyl)-2- oxo-5-(4,4,5,5- tetramethyl-1,3,2- dioxaborolan-2- yl)-1,2- dihydropyridine- 3-carboxylate | | |
| Intermediate 433 | | | |

### Example 1051

### methyl 6-(2-(3-chloro-4-fluorophenylamino)-4-(3-methoxypropylamino)pyrimidin-5-yl)-1H-indole-2-carboxylate

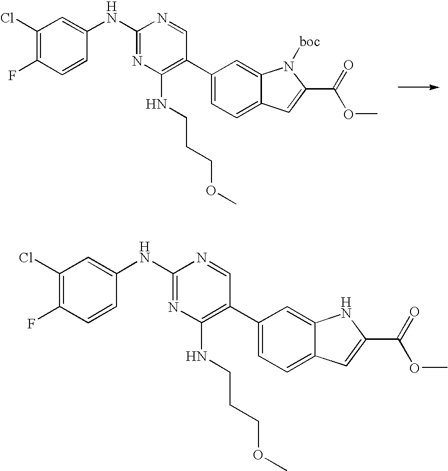

1-tert-butyl 2-methyl 6-(2-(3-chloro-4-fluorophenylamino)-4-(3-methoxypropylamino)pyrimidin-5-yl)-1H-indole-1,2-dicarboxylate (Example 983, 104 mg, 0.18 mmol) was suspended in DCM (4 mL). The solution was then treated with trifluoroacetic acid (0.274 ml, 3.56 mmol) and stirred at room temperature for 1 hr. The solvent was removed at reduced pressure and the residue was washed with Et₂O/hexanes to afford methyl 6-(2-(3-chloro-4-fluorophenylamino)-4-(3-methoxypropylamino)pyrimidin-5-yl)-1H-indole-2-carboxylate in 98% yield (84 mg).

MS(ES): 484 (M+1) for C₂₄H₂₃ClFN₅O₃.

¹H NMR (300 MHz, DMSO-D6) δ ppm 1.60-1.93 (m, 2H) 3.13 (s, 3H) 3.23-3.55 (m, 4H) 3.89 (s, 3H) 7.06 (dd, J=8.29, 1.32 Hz, 1H) 7.23 (d, J=0.94 Hz, 1H) 7.31-7.31-7.65 (m, 3H) 7.80 (dd, J=4.90, 3.39 Hz, 2H) 7.89-8.06 (m, 1H) 8.10 (s, 1H) 10.34 (s, 1H) 12.16 (s, 1H).

The following examples were prepared using the general method described above for Example 1051 using the starting material (SM) indicated.

| Ex | Compound | Data | SM |
|---|---|---|---|
| Example 1052 | 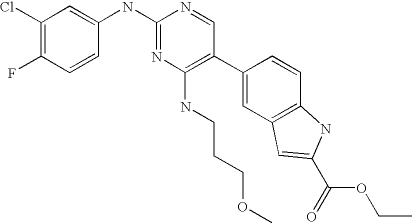 Ethyl 5-(2-(3-chloro-4- fluorophenylamino)-4- (3- methoxyproylamino)pyrimidin-5-yl)-1H- indole-2-carboxylate | MS(ES): 498 (M + 1H) for C₂₅H₂₅ClFN₅O₃ ¹H NMR (300 MHz, DMSO- D6) δ ppm 1.34 (t, J = 7.16 Hz, 3 H) 1.61-1.91 (m, 2 H) 3.12 (s, 3 H) 3.23-3.55 (m, 4 H) 4.35 (q, J = 6.97 Hz, 2 H) 7.14- 7.34 (m, 2 H) 7.34-7.65 (m, 3 H) 7.68 (s, 1 H) 7.77 (d, J = 1.51 Hz, 1 H) 7.84-8.12 (m, 2 H) 10.24 (s, 1 H) 10.87 (none, 1 H) 12.09 (s, 1 H) | 1-tert-butyl-2- ethyl 5-(2-(3- choloro-4- fluorophenyl- amino)-4-(3- methoxypro- pylamino)- pyrimidin-5-yl)- 1H-indole-1,2- dicarboxylate Example 984 |
| Example 1053 | 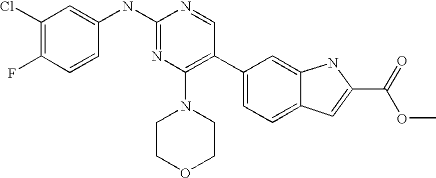 Methyl 6-(2-(3-chloro- 4-fluorophenylamino)- 4- morpholinopyrimidin- 5-yl)-1H-indole-2- carboxylate | MS(ES): 482 (M + 1) for C₂₄H₂₁ClFN₅O₃ ¹H NMR (300 MHz, DMSO- D6) δ ppm 3.14-3.42 (m, 4 H) 3.45-3.60 (m, 4 H) 3.87 (s, 3 H) 7.04-7.26 (m, 2 H) 7.36 (t, J = 9.04 Hz, 1 H) 7.51 (s, 1 H) 7.53-7.69 (m, 1 H) 7.72 (d, J = 8.29 Hz, 1 H) 7.94-8.21 (m, 2 H) 9.77 (s, 1 H) 12.04 (s, 1 H) | 1-tert-butyl 2- methyl 6-(2- (3-chloro-4- fluorophenyl- amino)-4- morpholino- pyrimidin-5- yl)-1H-indole- 1,2-dicarboxy- late Example 985 |
| Example 1054 | 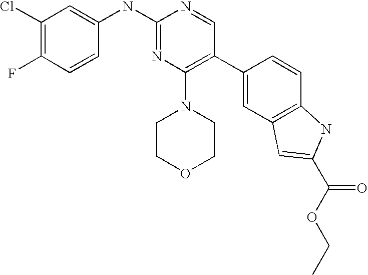 Ethyl 5-(2-(3-chloro-4- fluorophenylamino)-4- morpholinopyrimidin- 5-yl)-1H-indole-2- carboxylate | MS(ES): 496 (M + 1) for C₂₅H₂₃ClFN₅O₃ ¹H NMR (300 MHz, DMSO- D6) δ ppm 1.34 (t, J = 7.06 Hz, 3 H) 3.16-3.39 (m, 4 H) 3.42- 3.64 (m, 4 H) 4.34 (q, J = 6.97 Hz, 2 H) 7.09-7.26 (m, 1 H) 7.26-7.46 (m, 2 H) 7.44-7.66 (m, 2 H) 7.71 (s, 1 H) 7.89- 8.21 (m, 2 H) 9.84 (s, 1 H) 12.02 (s, 1 H) | 1-tert-butyl 2- ethyl 5-(2-(3- chloro-4- fluorophenyl- amino)-4- morpholino- pyrimidin-5-yl)- 1H-indole-1,2- dicarboxylate Example 986 |

The following examples were prepared using the general method described for Example 214 using 1N sodium hydroxide, THF/MeOH, and the starting material (SM) indicated.

| Ex | Compound | Data | SM |
|---|---|---|---|
| Example 1055 | 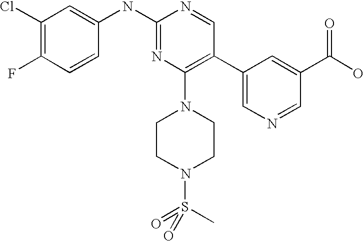 5-(2-(3-chloro-4- fluorophenylamino)-4-(4- (methylsulfonyl)piperazin- 1-yl)pyrimidin-5- yl)nicotinic acid | MS(ES): 507 (M + 1) for C₂₁H₂₀ClFN₆O₄S ¹H NMR (300 MHz, DMSO- D6) δ ppm 2.86 (s, 3 H) 2.98- 3.19 (m, 4 H) 3.29-3.47 (m, 4 H) 7.35 (t, J = 9.14 Hz, 1 H) 7.53-7.75 (m, 1 H) 8.12 (dd, J = 6.97, 2.64 Hz, 1 H) 8.20 (s, 1 H) 8.34 (t, J = 2.17 Hz, 1 H) 8.91 (d, J = 2.07 Hz, 1 H) 9.00 (d, J = 2.07 Hz, 1 H) 9.78 (s, 1 H) 13.56 (s, 1 H) | ethyl 5-(2-(3- chloro-4- fluorophenyl- amino)-4-(4- (methylsulfonyl) piperazin- 1- yl)pyrimidin- 5- yl)nicotinate Example 976 |
| Example 1056 | 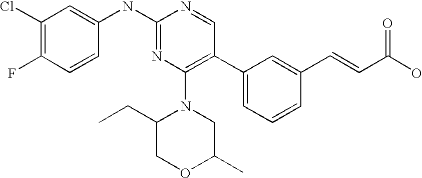 (E)-3-(3-(2-(3-chloro-4- fluorophenylamino)-4-(5- ethyl-2- methylmorpholino)pyrimidin-5-yl)phenyl)acrylic acid | MS(ES): 497 (M + 1) for C₂₆H₂₆ClFN₄O₃ ¹H NMR (300 MHz, DMSO- D6) δ ppm 0.56 (t, J = 7.35 Hz, 3 H) 1.02 (d, J = 6.03 Hz, 3 H) 1.35-1.83 (m, 2 H) 2.65-2.87 (m, 1 H) 3.40- 3.86 (m, 5 H) 6.60 (d, J = 16.01 Hz, 1 H) 7.33 (t, J = 9.14 Hz, 1 H) 7.40-7.70 (m, 5 H) 7.74 (s, 1 H) 7.99 (s, 1 H) 8.17 (dd, H = 6.88, 2.35 Hz, 1 H) 9.63 (s, 1 H) 12.45 (s,1 H) | (E)-ethyl 3-(3- (2-(3-chloro-4- fluorophenyl- amino)-4-(5- ethyl-2- methylmorpholino) pyrimidin- 5- yl)phenyl) acrylate Example 979 |
| Example 1057 | 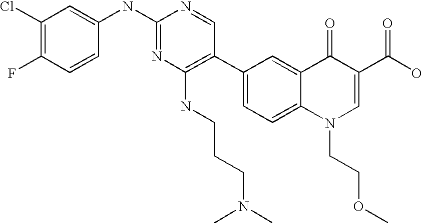 6-(2-(3-Chloro-4- fluorophenylamino)-4-(3- (dimethylamino)propyl- amino)pyrimidin-5-yl)-1-(2- methoxyethyl)-4-oxo-1,4- dihydroquinoline-3- carboxylic acid | MS(ES): 569 (M + 1) for C₂₈H₃₀ClFN₆O₄ ¹H NMR (300 MHz, DMSO- D6) δ ppm 1.82-2.07 (m, 2 H) 2.78 (s, 6 H) 2.99-3.16 (m, 2 H) 3.24 (s, 3 H) 3.35- 3.52 (m, 2 H) 3.61-3.82 (m, 2 H) 4.84 (t, J = 4.52 Hz, 2 H) 7.40 (t, J = 9.04 Hz, 1 H) 7.51- 7.69 (m, 1 H) 7.86-8.07 (m, 2 H) 8.04-8.33 (m, 2 H) 8.41 (d, J = 2.07 Hz, 1 H) 8.97 (s, 1 H) 9.35 (s, 1 H) 9.95 (s, 1 H) 15.14 (s, 1 H) | Ethyl 6-(2-(3- chloro-4- fluorophenyl- amino)-4-(3- (dimethylamino) propylamino) pyrimidin-5-yl)- 1-(2- methoxyethyl)- 4-oxo-1,4- dihydroquinline- 3-carboxylate Example 980 |
| Example 1058 | 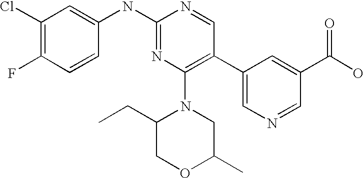 5-(2-(3-Chloro-4- fluorophenylamino)-4-(5- ethyl-2- methylmorpholino)pyrimidin- 5-yl)nicotinic acid | MS(ES): 472 (M + 1) for C₂₃H₂₃ClFN₅O₃ ¹H NMR (300 MHz, DMSO- D6) δ ppm 0.58 (t, J = 7.44 Hz, 3 H) 1.03 (d, J = 5.84 Hz, 3 H) 1.39-1.86 (m, 2 H) 2.78 (dd, J = 13.47, 11.21 Hz, 1 H) 3.36-3.71 (m, 5 H) 7.16-7.45 (m, 1 H) 7.45- 7.73 (m, 1 H) 8.00-8.25 (m, 2 H) 8.29 (t, J = 2.07 Hz, 1 H) 8.86 (d, J = 2.07 Hz, 1 H) 9.00 (d, J = 1.88 Hz, 1 H) 9.69 (s, 1 H) 13.53 (s, 1 H) | Ethyl 5-(2-(3- chloro-4- fluorophenyl- amino)-4-(5-ethyl- 2- methylmorpholino) pyrimidin-5- yl)nicotinate Example 981 |
| Example 1059 | 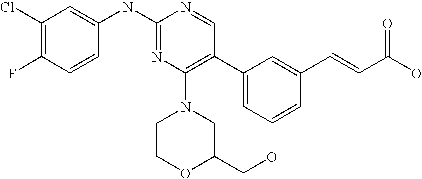 (E)-3-(3-(2-(3-chloro-4- fluorophenylamino)-4-(2- (hydroxyethyl)morpholino)pyrimidin-5- yl)phenyl)acrylic acid | MS(ES): 485 (M + 1) for C₂₄H₂₂ClFN₄O₄ ¹H NMR (300 MHz, DMSO- D6) δ ppm 2.56-2.98 (m, 2 H) 3.16-3.58 (m, 5 H) 3.59- 4.00 (m, 2 H) 4.48-5.01 (m, 1 H) 6.58 (d, J = 16.01 Hz, 1 H) 7.12-7.85 (m, 7 H) 7.96-8.27 (m, 2 H) 9.62 (s, 1 H) | (E)-ethyl 3-(3- (2-(3-chloro-4- fluorophenylamino)-4-(2- (hydroxymethyl) morpholino) pyrimidin-5- yl)phenyl)- acrylate Example 977 |
| Example 1060 | 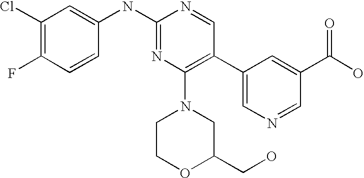 5-(2-(3-Chloro-4- fluorophenylamino)-4-(2- hydroxymethyl)morpholin)pyrimidin-5-yl)nicotinic acid | MS(ES): 460 (M + 1) for C₂₁H₁₉ClFN₅O₄ ¹H NMR (300 MHz, DMSO- D6) δ ppm 2.57-2.79 (m, 1 H) 2.77-3.04 (m, 1 H) 3.12- 3.59 (m, 5 H) 3.59-3.90 (m, 2 H) 4.47-4.83 (m, 1 H) 7.32 (t, J = 9.14 Hz, 1 H) 7.54- 7.82 (m, 1 H) 8.03-8.22 (m, 2 H) 8.32 (t, J = 2.07 Hz, 1 H) 8.88 (d, J = 2.26 Hz, 1 H) 8.98 (d, J = 1.88 Hz, 1 H) 9.73 (s, 1 H) 13.54 (s, 1 H) | Ethyl 5-(2-(3- chloro-4- fluorophenylamino)- 4-(2- (hydroxymethyl) morpholino) pyrimidin-5- yl)nicotinate Example 978 |
| Example 1061 | 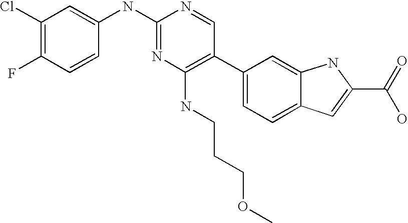 6-(2-(3-chloro-4- fluorophenylamino)-4-(3- methoxypropylamino)pyrimidin-5-yl)-1H-indole- 2-carboxylic acid | MS(ES): 470 (M + 1) for C₂₃H₂₁ClFN₅O₃ ¹H NMR (300 MHz, DMSO- D6) δ ppm 1.66-1.93 (m, 2 H) 3.13 (s, 3 H) 3.33-3.56 (m, 4 H) 6.54 (t, J = 5.46 Hz, 1 H) 6.68 (s, 1 H) 6.93 (dd, J = 8.19, 1.22 Hz, 1 H) 7.15- 7.43 (m, 2 H) 7.50-7.74 (m, 2 H) 7.77 (s, 1 H) 8.25 (dd, J = 7.06, 2.54 Hz, 1 H) 9.42 (s, 1 H) 11.19 (s, 1 H) | Methyl 6-(2-(3- chloro-4- fluorophenyl- amino)-4-(3- methoxypropyl- amino)pyrimidin- 5-yl)-1H-indole- 2-carboxylate Example 1051 |
| Example 1062 | 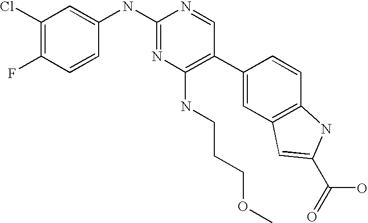 5-(2-(3-chloro-4- fluorophenylamino)-4-(3- methoxypropylamino)pyrimidin-5-yl)-1H-indole- 2-carboxylic acid | MS(ES): 470 (M + 1) for C₂₃H₂₁ClFN₅O₃ ¹H NMR (300 MHz, DMSO- D6) δ ppm 1.62-1.94 (m, 2 H) 3.13 (s, 3 H) 3.22-3.60 (m, 4 H) 6.90 (s, 1 H) 7.12 (s, 1 H) 7.21 (dd, J = 8.48, 1.51 Hz, 1 H) 7.32 (t, J = 9.14 Hz, 1 H) 7.51 (d, J = 8.48 Hz, 1 H) 7.55-7.70 (m, 2 H) 7.76 (s, 1 H) 8.02-8.33 (m, 1 H) 9.55 (s, 1 H) 11.87 (s, 1 H) 13.03 (s, 1 H) | Ethyl 5-(2-(3- chloro-4- fluorophenyl- amino)-4-(3- methoxypropyl- amino)pyrimidin- 5-yl)-1H-indole- 2-carboxylate Example 1052 |
| Example 1063 | 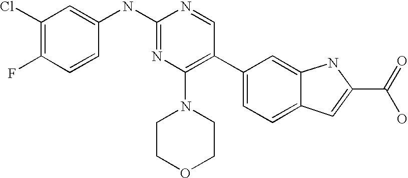 6-(2-(3-Chloro-4- fluorophenylamino)-4- morpholinopyrimidin-5- yl)-1H-indole-2-carboxylic acid | MS(ES): 468 (M + 1) for C₂₃H₁₉ClFN₅O₃ ¹H NMR (300 MHz, DMSO- D6) δ ppm 3.10-3.26 (m, 4 H) 3.45-3.65 (m, 4 H) 6.74- 7.04 (m, 1 H) 7.12 (dd, J = 8.38, 1.22 Hz, 1 H) 7.31 (t, J = 9.23 Hz, 1 H) 7.48 (s, 1 H) 7.56-7.78 (m, 2 H) 8.03 (s, 1 H) 8.16 (dd, J = 6.97, 2.64 Hz, 1 H) 9.57 (s, 1 H) 11.58 (s, 1 H) | Methyl 6-(2-(3- chloro-4- fluorophenyl- amino)-4- morpholino- pyrimidin-5-yl)-1H- indole-2- carboxylate Example 1053 |
| Example 1064 | 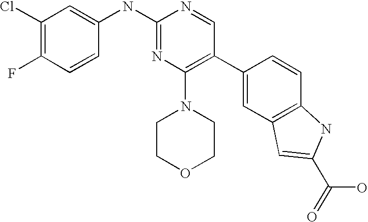 5-(2-(3-Chloro-4- fluorophenylamino)-4- morpholinopyrimidin-5- yl)-1H-indole-2-carboxylic acid | MS(ES): 468 (M + 1) for C₂₃H₁₉ClFN₅O₃ ¹H NMR (300 MHz, DMSO- D6) δ ppm 3.13-3.26 (m, 4 H) 3.42-3.69 (m, 4 H) 7.08 (s, 1 H) 7.21-7.41 (m, 2 H) 7.47 (d, J = 8.67 Hz, 1 H) 7.55- 7.84 (m, 2 H) 8.02 (s, 1 H) 8.15 (dd, J = 6.69, 2.35 Hz, 1 H) 9.54 (s, 1 H) 11.81 (s, 1 H) 12.99 (s, 1 H) | Ethyl 5-(2-(3- chloro-4- fluorophenylamino)- 4- morpholino- pyrimidin-5-yl)-1H- indole-2- carboxylate Example 1054 |
| Example 1065 | 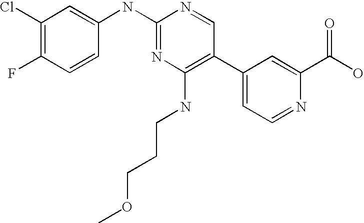 4-(2-(3-Chloro-4- fluorophenylamino)-4-(3- methoxypropylamino)pyrimidin-5-yl)nicotinic acid | MS(ES): 432 (M + 1) for C₂₀H₁₉ClFN₅O₃ ¹H NMR (300 MHz, DMSO- d₆) δ ppm 1.70-1.96 (m, 2 H) 3.20 (s, 3 H) 3.35-3.55 (m, 4 H) 6.89 (t, J = 5.75 Hz, 1 H) 7.17-7.45 (m, 2 H) 7.57-7.71 (m, 1 H) 7.86 (s, 1 H) 7.89 (s, 1 H) 8.22 (dd, J = 6.88, 2.54 Hz, 1 H) 8.53 (d, J = 4.71 Hz, 1 H) 9.50 (s, 1 H) | Methyl 4-(2-(3- chloro-4- fluorophenyl- amino)-4-(3- methoxypropyl- amino)pyrimidin- 5-yl)picolinate Example 1031 |

Methyl-ester Hydrolysis: The following examples were prepared using the general method described for Example 214 using 1N sodium hydroxide, THF : 1,4-dioxane (1:1), and the starting material (SM) indicated.

| Ex | Compound | Data | SM |
|---|---|---|---|
| Example 1066 | 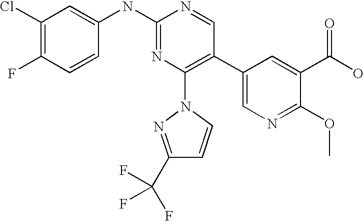 5-(2-(3-chloro-4- fluorophenylamino)-4-(3- (trifluoromethyl)-1H- pyrazol-1-yl)pyrimidin-5- yl)-2-methoxynicotinic acid | MS(ES): 509 (M + 1) for C₂₁H₁₃ClF₄N₆O₃ ¹H NMR (300 MHz, DMSO-d₆) δ ppm 3.93 (s, 3 H) 7.04 (d, J = 2.45 Hz, 1 H) 7.42 (t, J = 9.14 Hz, 1 H) 7.64-7.78 (m,1 H) 7.82 (d, J = 2.45 Hz, 1 H) 8.08 (dd, J = 6.69, 2.54 Hz, 1 H) 8.24 (d, J = 2.45 Hz, 1 H) 8.50 (s, 1 H) 8.80 (s, 1 H) 10.41 (s, 1 H) 12.95 (br. s., 1 H) | Methyl 5-(2-(3- chloro-4- fluorophenyl- amino)-4-(3- (trifluoromethyl)- 1H-pyrazol-1- yl)pyrimidin-5- yl)-2- methoxynicotinate Example 991 |
| Example 1067 | 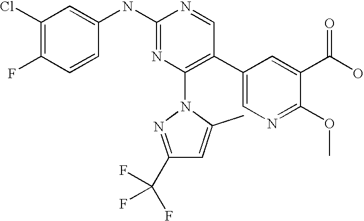 5-(2-(3-chloro-4- fluorophenylamino)-4-(5- methyl-3- (trifluoromethyl)-1H- pyrazol-1-yl)pyrimidin- 5-yl)-2-methoxynicotinic acid | MS(ES): 523 (M + 1) for C₂₂H₁₅ClF₄N₆O₃ ¹H NMR (300 MHz, DMSO-d₆) δ ppm 2.34 (s, 3 H) 3.90 (s, 3 H) 6.76 (s, 1 H) 7.42 (t, J = 9.14 Hz, 1 H) 7.54-7.78 (m, 2 H) 8.06 (d, J = 6.78 Hz, 1 H) 8.17 (d, J = 2.45 Hz, 1 H) 8.96 (s, 1 H) 10.44 (s, 1 H) 12.97 (br. s., 1 H) | Methyl 5-(2-(3- chloro-4- fluorophenyl- amino)-4-(5- methyl-3-(trifluoro- methyl)-1H-pyrazol- 1-yl)pyrimidin-5- yl)-2- methoxynicitnate Example 992 |
| Example 1068 | 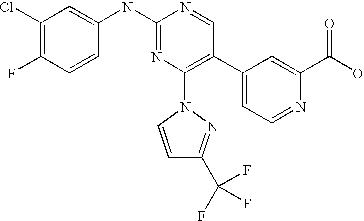 4-(2-(3-Chloro-4- fluorophenylamino)-4-(3- (trifluoromethyl)-1H- pyrazol-1-yl)pyrimidin-5- yl)picolinic acid | MS(ES): 479 (M + 1) for C₂₀H₁₁ClF₄N₆O₂ ¹H NMR (300 MHz, DMSO-d₆) δ ppm 7.07 (d, 1 H) 7.30-7.60 (m, 2 H) 7.63-7.83 (m, 2 H) 8.08 (dd, J = 6.69, 2.54 Hz, 1 H) 8.55 (d, J = 1.70 Hz, 1 H) 8.67 (d, J = 5.09 Hz, 1 H) 8.85 (s, 1 H) 10.53 (s, 1 H) | Methyl 4-(2-(3- chloro-4- fluorophenyl- amino)-4-(3- (trifluoromethyl)- 1H-pyrazol-1- yl)pyrimidin-5- yl)picolinate Example 1032 |
| Example 1069 | 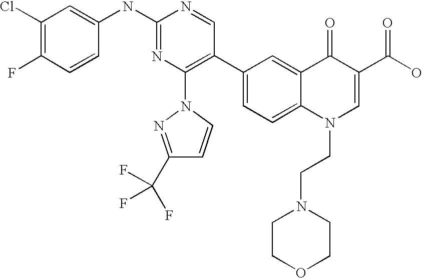 6-(2-(3-Chloro-4- fluorophenylamino)-4-(3- (trifluoromethyl)-1H- pyrazol-1-yl)pyrimidin-5- yl)-1-(2-morpholinoethyl)- 4-oxo-1,4- dihydroquinoline-3- carboxylic acid | MS(ES): 658 (M + 1) for C₃₀H₂₄ClF₄N₇O₄ 1H NMR (300 MHz, DMSO-d₆) δ ppm 2.79- 3.34 (m, 4 H) 3.31-4.22 (m, 6 H) 4.77-5.19 (m, 2 H) 7.04 (d, J = 2.64 Hz, 1 H) 7.44 (t, J = 9.14 Hz, 1H) 7.60-7.89 (m, 2 H) 8.01 (d, J = 9.04 Hz, 1 H) 8.13 (dd, J = 6.78, 2.64 Hz, 1 H) 8.20 (d, J = 2.07 Hz, 1 H) 8.47 (d, J = 1.51 Hz, 1 H) 8.88 (s, 1 H) 9.10 (s, 1 H) 10.49 (s, 1 H) 15.03 (br. s., 1 H) | Ethyl 6-(2-(3- chloro-4- fluorophenyl- amino)-4-(3- (trifluoromethyl)- 1H-pyrazol-1- yl)pyrimidin-5- yl)-1-(2- morpholinoethyl)- 4-oxo-1,4- dihydroquinoline- 3-carboxylate Example 1037 |
| Example 1070 | 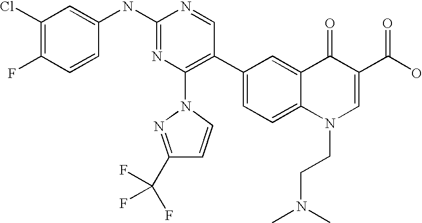 6-(2-(3-Chloro-4- fluorophenylamino)-4-(3- (trifluoromethyl)-1H- pyrazol-1-yl)pyrimidin-5- yl)-1-(2- (dimethylamino)ethyl)-4- oxo-1,4-dihydroquinoline- 3-carboxylic acid | MS(ES): 616 (M + 1) for C₂₈H₂₂ClF₄N₇O₃ ¹H NMR (300 MHz, DMSO-d₆) δ ppm 2.89 (br. s., 6 H) 3.53-3.74 (m, 2 H) 4.93 (t, J = 5.93 Hz, 2 H) 7.04 (d, J = 2.83 Hz, 1 H) 7.44 (t, J = 9.04 Hz, 1 H) 7.60-7.88 (m, 2 H) 8.02 (d, J = 9.04 Hz, 1 H) 8.07- 8.28 (m, 2 H) 8.47 (d, J = 1.70 Hz, 1 H) 8.88 (s, 1 H) 9.14 (s, 1 H) 10.50 (s, 1 H) 15.01 (br. s., 1 H) | Ethyl 6-(2-(3- chloro-4- fluorophenyl- amino)-4-(3- (trifluoromethyl)- 1H-pyrazol-1- yl)pyrimidin-5- yl)-1-(2- (dimethylamino) ethyl)-4-oxo- 1,4- dihydroquinoline- 3-carboxylate Example 1038 |
| Example 1071 | 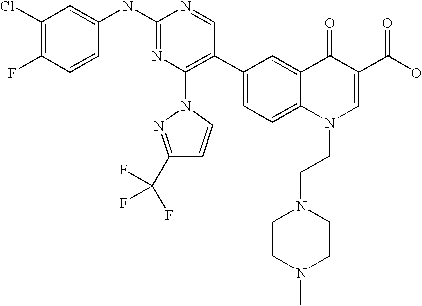 6-(2-(3-Chloro-4- fluorophenylamino)-4-(3- (trifluoromethyl)-1H- pyrazol-1-yl)pyrimidin-5- yl)-1-(2-(4- methylpiperazin-1- yl)ethyl)-4-oxo-1,4- dihydroquinoline-3- carboxylic acid | MS(ES): 671 (M + 1) for C₃₁H₂₇ClF₄N₈O₃ ¹H NMR (300 MHz, DMSO-d₆) δ ppm 2.39 (t, 2 H) 2.63-2.92 (m, 7 H) 3.00 (d, J = 13.37 Hz, 2 H) 3.38 (d, J = 11.11 Hz, 2 H) 4.71 (t, J = 4.99 Hz, 2 H) 7.03 (d, J = 2.64 Hz, 1 H) 7.44 (t, J = 9.04 Hz, 1 H) 7.60-7.83 (m, 2 H) 8.04 (d, J = 9.04 Hz, 1 H) 8.12 (dd, J = 6.78, 2.64 Hz, 1 H) 8.21 (d, J = 2.07 Hz, 1 H) 8.50 (d, J = 1.51 Hz, 1 H) 8.89 (s, 1 H) 8.98 (s, 1 H) 10.48 (s, 1 H) | Ethyl 6-(2-(3- chloro-4- fluorophenyl- amino)-4-(3- (trifluoromethyl)- 1H-pyrazol-1- yl)pyrimidin-5- yl)-1-(2-(4- methylpiperazin- 1-yl)ethyl)-4- oxo-1,4- dihydroquinoline- 3-carboxylate Example 994 |
| Example 1072 | 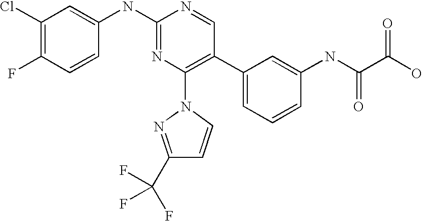 2-(3-(2-(3-Chloro-4- fluorophenylamino)-4-(3- (trifluoromethyl)-1H- pyrazol-1-yl)pyrimidin-5- yl)phenylamino)-2- oxoacetic acid | MS(ES): 521 (M + 1) for C₂₂H₁₃ClF₄N₆O₃ ¹H NMR (300 MHz, DMSO-d₆) δ ppm 6.89 (d, 1 H) 6.97 (d, J = 2.64 Hz, 1 H) 7.19-7.53 (m, 2 H) 7.54-7.88 (m, 3 H) 8.12 (dd, J = 6.78, 2.64 Hz, 1H) 8.29 (d, J = 1.70 Hz, 1 H) 8.73 (s, 1 H) 10.39 (s, 1 H) 10.70 (s, 1 H) 14.19 (br. s., 1 H) | Ethyl 2-(3-(2-(3- chloro-4- fluorophenyl- amino)-4-(3- (trifluoromethyl)- 1H-pyrazol-1- yl)pyrimidin-5- yl)phenylamino)- 2-oxoacetate Example 996 |
| Example 1073 | 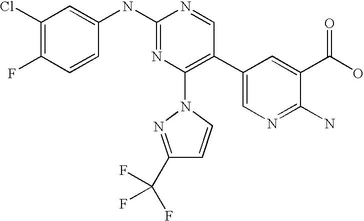 2-Amino-5-(2-(3-chloro-4- fluorophenylamino)-4-(3- (trifluoromethyl)-1H- pyrazol-1-yl)pyrimidin-5- yl)nicotinic acid | MS(ES): 494 (M + 1) for C₂₀H₁₂ClF₄N₇O₂ ¹H NMR (300 MHz, DMSO-d₆) δ ppm 7.03 (d, 1 H) 7.41 (t, J = 9.14 Hz, 1 H) 7.70 (ddd, J = 9.09, 4.19, 2.73 Hz, 1 H) 7.86 (d, J = 2.45 Hz, 1 H) 7.99-8.19 (m, 2 H) 8.46 (d, J = 1.70 Hz, 1 H) 8.77 (s, 1 H) 10.35 (s, 1 H) | Methyl 2-amino- 5-(2-(3-chloro- 4- fluorophenyl- amino)-4-(3- (trifluoromethyl)- 1H-pyrazol-1- yl)pyrimidin-5- yl)nicotinate Example 999 |
| Example 1074 | 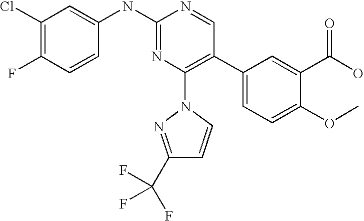 5-(2-(3-Chloro-4- fluorophenylamino)-4-(3- (trifluoromethyl)-1H- pyrazol-1-yl)pyrimidin-5- yl)-2-methoxybenzoic acid | MS(ES): 508 (M + 1) for C₂₂H₁₄ClF₄N₅O₃ ¹H NMR (300 MHz, DMSO-d₆) δ ppm 3.82 (s, 3 H) 6.97 (d, J = 2.64 Hz, 1 H) 7.12 (d, J = 8.85 Hz, 1 H) 7.21-7.53 (m, 3 H) 7.70 (ddd, J = 9.09, 4.19, 2.73 Hz, 1 H) 8.12 (dd, J = 6.78, 2.64 Hz, 1 H) 8.30 (d, J = 1.51 Hz, 1 H) 8.76 (s, 1 H) 10.36 (s, 1 H) 12.56 (br. s., 1 H) | Methyl 5-(2-(3- chloro-4- fluorophenyl- amino)-4-(3- (trifluoromethyl)- 1H-pyrazol-1- yl)pyrimidin-5- yl)-2-methoxy benzoate Example 1001 |
| Example 1075 | 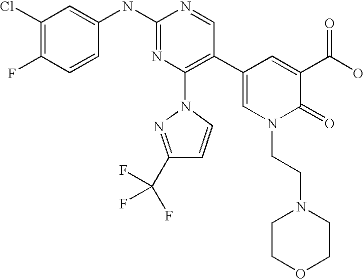 5-(2-(3-Chloro-4- fluorophenylamino)-4-(3- (trifluoromethyl)-1H- pyrazol-1-yl)pyrimidin-5- yl)-1-(2-morpholinoethyl)- 2-oxo-1,2- dihydropyridine-3- carboxylic acid | MS(ES): 608 (M + 1) for C₂₆H₂₂ClF₄N₇O₄ ¹H NMR (300 MHz, CHLOROFORM-d) δ ppm 2.80-3.77 (m, 6 H) 4.02 (br. s., 4 H) 4.67 (t, J = 5.93 Hz, 2 H) 6.73 (d, J = 2.64 Hz, 1 H) 7.20 (t, J = 8.67 Hz, 1 H) 7.38 (dd, J = 3.77, 2.83 Hz, 1 H) 7.83 (dd, J = 6.41, 2.64 Hz, 1 H) 7.97 (br. s., 1 H) 8.14 (br. s., 1 H) 8.32-8.49 (m, 2 H) 8.52 (s, 1 H) | Methyl 5-(2-(3- chloro-4- fluorophenyl- amino)-4-(3- (trifluoromethyl)- 1H-pyrazol-1- yl)pyrimidin-5- yl)-1-(2- morpholinoethyl)- 2-oxo-1,2- dihydropyridine- 3-carboxylate Example 1004 |
| Example 1076 | 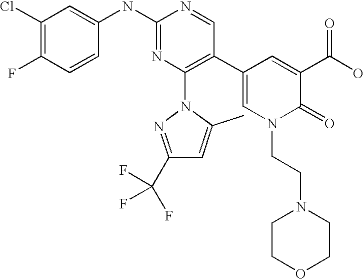 5-(2-(3-Chloro-4- fluorophenylamino)-4-(5- methyl-3- (trifluoromethyl)-1H- pyrazol-1-yl)pyrimidin-5- yl)-1-(2-morpholinoethyl)- 2-oxo-1,2- dihydrpyridine-3- carboxylic acid | MS(ES): 622 (M + 1) for C₂₇H₂₄ClF₄N₇O₄ ¹H NMR (300 MHz, CHLOROFORM-d) δ ppm 2.58 (s, 3 H) 2.90-3.66 (m, 6 H) 4.01 (br. s., 4 H) 4.61 (br. s., 2 H) 6.74 (s, 1 H) 7.16 (t, J = 8.57 Hz, 1 H) 7.31-7.46 (m, 1 H) 7.66 (br. s., 1 H) 7.81 (d, J = 3.96 Hz, 2 H) 8.17 (br. s., 1 H) 8.56 (br. s., 1 H) | Methyl 5-(2-(3- chloro-4- fluorophenyl- amino)-4-(5-methyl- 3- (trifluoromethyl)- 1H-pyrazol-1- yl)pyrimidin-5- yl)-1-(2- morpholinoethyl)- 2-oxo-1,2- dihydrpyridine- 3-carboxylate Example 1005 |
| Example 1077 | 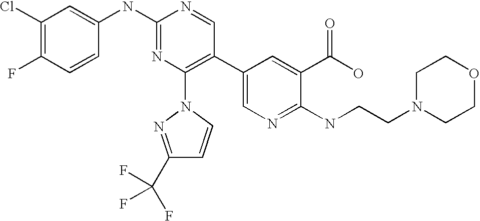 5-(2-(3-Chloro-4- fluorophenylamino)-4-(3- (trifluoromthyl)-1H- pyrazol-1-yl)pyrimidin-5- yl)-2-(2- morpholinoethylamino)nicotinic acid | MS(ES): 607 (M + 1) for C₂₆H₂₃ClF₄N₈O₃ ¹H NMR (300 MHz, DMSO-d₆) δ ppm 3.10- 4.10 (m, 12 H) 7.03 (d, J = 2.64 Hz, 1 H) 7.41 (t, J = 9.14 Hz, 1 H) 7.70 (ddd, J = 9.04, 4.24, 2.73 Hz, 1 H) 7.83 (d, J = 2.45 Hz, 1 H) 8.10 (dd, J = 6.78, 2.64 Hz, 1 H) 8.19 (d, J = 2.45 Hz, 1 H) 8.32 (s, 1 H) 8.46 (d, J = 1.51 Hz, 1 H) 8.78 (s,1 H) 10.35 (s, 1 H) | Methyl 5-(2-(3- chloro-4- fluorophenyl- amino)-4-(3- (trifluoromethyl)- 1H-pyrazol-1- yl)pyrimidin-5- yl)-2-(2- morpholinoethyl amino)nicotinate Example 1006 |
| Example 1078 | 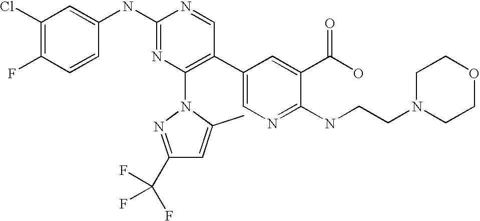 5-(2-(3-Chloro-4- fluorophenylamino)-4-(5- methyl-3- (trifluoromethyl)-1H- pyrazol-1-yl)pyrimidin- 5-yl)-2-(2- morpholinoethylamino)nicotinic acid | MS(ES): 621 (M + 1) for C₂₇H₂₅ClF₄N₈O₃ ¹H NMR (300 MHz, DMSO-d₆) δ ppm 2.26 (s, 3 H) 2.88-4.20 (m, 12 H) 6.75 (s, 1 H) 7.41 (t, J = 9.14 Hz, 1 H) 7.54-7.78 (m, 2 H) 7.98-8.22 (m, 2 H) 8.20-8.44 (m, 1 H) 8.93 (s, 1 H) 9.63 (br. s., 1 H) 10.39 (s, 1 H) | Methyl 5-(2-(3- chloro-4- fluorophenyl- amino)-4-(5-methyl- 3- (trifluoromethyl)- 1H-pyrazol-1- yl)pyrimidin-5- yl)-2-(2- morpholinoethyl amino)nicotinate Example 1007 |
| Example 1079 | 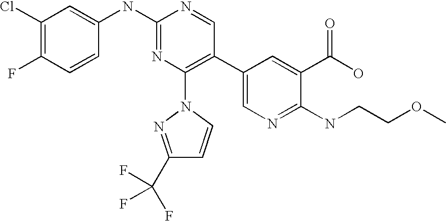 5-(2-(3-Chloro-4- fluorophenylamino)-4-(3- (trifluoromethyl)-1H- pyrazol-1-yl)pyrimidin- 5-yl)-2-(2- methoxyethylamino)nicotinic acid | MS(ES): 552 (M + 1) for C₂₃H₁₈ClF₄N₇O₃ ¹H NMR (300 MHz, DMSO-d₆) δ ppm 3.27 (s, 3 H) 3.42-3.67 (m, 4 H) 6.97 (d, J = 2.64 Hz, 1 H) 7.39 (t, J = 9.14 Hz, 1 H) 7.60-7.80 (m, 2 H) 7.87 (br. s., 1 H) 8.12 (dd, J = 6.78, 2.64 Hz, 1 H) 8.30 (s, 1 H) 8.73 (s, 1 H) 9.29 (br. s., 1 H) 10.31 (s, 1 H) | Methyl 5-(2-(3- chloro-4- fluorophenyl- amino)-4-(3- (trifluoromethyl)- 1H-pyrazol-1- yl)pyrimidin-5- yl)-2-(2- methoxyethyl- amino)nicotinate Example 1011 |
| Example 1080 | 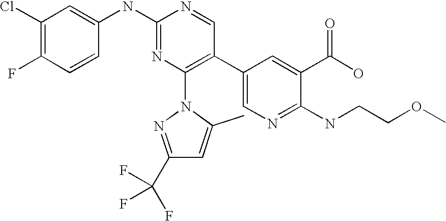 5-(2-(3-Chloro-4- fluorophenylamino)-4-(5- methyl-3- (trifluoromethyl)-1H- pyrazol-1-yl)pyrimidin- 5-yl)-2-(2- methoxyethylamino)nicotinic acid | MS(ES): 566 (M + 1) for C₂₄H₂₀ClF₄N₇O₃ ¹H NMR (300 MHz, DMSO-d₆) δ ppm 2.19 (s, 3 H) 3.26 (s, 3 H) 3.39- 3.66 (m, 4 H) 6.71 (s, 1 H) 7.39 (t, J = 9.14 Hz, 1 H) 7.52-7.88 (m, 3 H) 8.06 (dd, J = 6.78, 2.45 Hz, 1 H) 8.86 (s, 1 H) 9.24 (s, 1 H) 10.33 (s, 1 H) 13.09 (s, 1 H) | Methyl 5-(2-(3- chloro-4- fluorophenyl- amino)-4- (5-methyl-3- (trifluoromethyl)- 1H-pyrazol-1- yl)pyrimidin-5- yl)-2-(2- methoxyethyl- amino)nicotinate Example 1012 |
| Example 1081 | 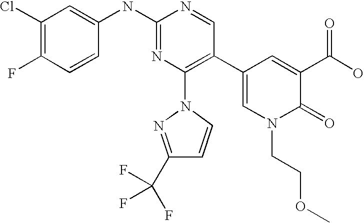 5-(2-(3-Chloro-4- fluorophenylamino)-4-(3- (trifluoromethyl)-1H- pyrazol-1-yl)pyrimidin- 5-yl)-1-(2- methoxyethyl)-2-oxo- 1,2-dihydropyridine-3- carboxylic acid | MS(ES): 553 (M + 1) for C₂₃H₁₇ClF₄N₆O₄ ¹H NMR (300 MHz, DMSO-d₆) δ ppm 3.23 (s, 3 H) 3.68 (t, J = 5.27 Hz, 2 H) 4.29 (t, J = 5.37 Hz, 2 H) 7.08 (d, J = 2.64 Hz, 1 H) 7.43 (t, J = 9.04 Hz, 1 H) 7.72 (ddd, J = 6.83, 4.47, 2.07 Hz, 1 H) 7.98-8.18 (m, 2 H) 8.27 (d, J = 2.64 Hz, 1 H) 8.60 (d, J = 1.70 Hz, 1 H) 8.71 (s, 1 H) 10.40 (s, 1 H) 14.34 (br. s., 1 H) | Ethyl 5-(2-(3- chloro-4- fluorophenyl- amino)-4-(3- (trifluoromethyl)- 1H-pyrazol-1- yl)pyrimidin-5- yl)-1-(2- methoxyethyl)- 2-oxo-1,2- dihydropyridine- 3-carboxylate Example 1013 |
| Example 1082 | 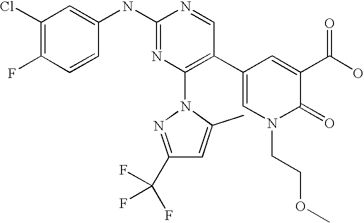 5-(2-(3-Chloro-4- fluorophenylamino)-4-(5- methyl-3- (trifluoromethyl)-1H- pyrazol-1-yl)pyrimidin-5- yl)-1-(2-methoxyethyl)-2- oxo-1,2-dihydropyridine- 3-carboxylic acid | MS(ES): 567 (M + 1) for C₂₄H₁₉ClF₄N₆O₄ 1H NMR (300 MHz, DMSO-d₆) δ ppm 2.47 (s, 3 H) 3.23 (s, 3 H) 3.65 (t, J = 5.09 Hz, 2 H) 4.27 (t, J = 4.99 Hz, 2 H) 6.80 (s, 1 H) 7.42 (t, J = 9.04 Hz, 1 H) 7.55-7.71 (m, 1 H) 7.75 (d, J = 2.35 Hz, 1 H) 8.05 (dd, J = 6.59, 2.45 Hz, 1 H) 8.25 (d, J = 2.45 Hz, 1 H) 8.85 (s, 1 H) 10.41 (s, 1 H) 14.18 (br. s., 1 H) | Ethyl 5-(2-(3- chloro-4- fluorophenyl- amino)-4- (5-methyl-3- (trifluoromethyl)- 1H-pyrazol-1- yl)pyrimidin-5- yl)-1-(2- methoxyethyl)- 2-oxo-1,2- dihydrpyiridine- 3-carboxylate Example 1014 |
| Example 1083 | 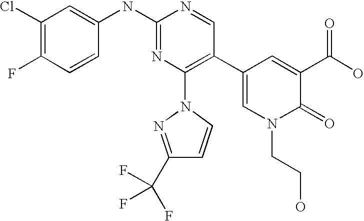 5-(2-(3-Chloro-4- fluorophenylamino)-4-(3- (trifluoromethyl)-1H- pyrazol-1-yl)pyrimidin-5- yl)-1-(2-hydroxyethyl)-2- oxo-1,2-dihydropyridine- 3-carboxylic acid | MS(ES): 539 (M + 1) for C₂₂H₁₅ClF₄N₆O₄ ¹H NMR (300 MHz, DMSO-d₆) δ ppm 3.73 (t, J = 5.56 Hz, 2 H) 4.19 (t, J = 5.56 Hz, 2 H) 7.08 (d, J = 2.64 Hz, 1 H) 7.42 (t, J = 9.14 Hz, 1 H) 7.72 (ddd, J = 9.09, 4.19, 2.73 Hz, 1 H) 7.96-8.17 (m, 2 H) 8.28 (d, J = 2.64 Hz, 1 H) 8.51- 8.63 (m, 1 H) 8.71 (s, 1 H) 10.41 (s, 1 H) | Ethyl 5-(2-(3- chloro-4- fluorophenyl- amino)-4-(3- (trifluoromethyl)- 1H-pyrazol-1- yl)pyrimidin-5- yl)-1-(2- hydroxyethyl)-2- oxo-1,2- dihydropyridine- 3-carboxylate Example 1015 |
| Example 1084 | 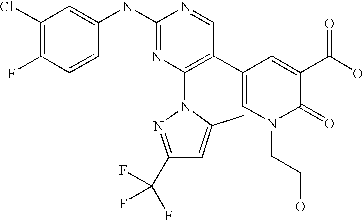 5-(2-(3-Chloro-4- fluorophenylamino)-4-(5- methyl-3- (trifluoromethyl)-1H- pyrazol-1-yl)pyrimidin- 5-yl)-1-(2-hydroxyethyl)- 2-oxo-1,2- dihydropyridine-3- carboxylic acid | MS(ES): 553 (M + 1) for C₂₃H₁₇ClF₄N₆O₄ ¹H NMR (300 MHz, DMSO-d₆) δ ppm 2.45 (s, 3 H) 3.71 (t, J = 5.37 Hz, 2 H) 4.16 (t, J = 5.46 Hz, 2 H) 6.80 (s, 1 H) 7.42 (t, J = 9.14 Hz, 1 H) 7.55-7.79 (m, 2 H) 8.05 (dd, J = 6.78, 2.64 Hz, 1 H) 8.25 (d, J = 2.83 Hz, 1 H) 8.86 (s, 1 H) 10.42 (s, 1 H) 14.27 (br. s., 1 H) | Ethyl 5-(2-(3- chloro-4- fluorophenyl- amino)-4-(5- methyl-3- (trifluoromethyl)- 1H-pyrazol-1- yl)pyrimidin-5- yl)-1-(2- hydroxyethyl)-2- oxo-1,2- dihydropyridine- 3-carboxylate Example 1043 |
| Example 1085 | 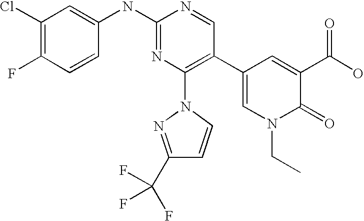 5-(2-(3-Chloro-4- fluorophenylamino)-4-(3- (trifluoromethyl)-1H- pyrazol-1-yl)pyrimidin-5- yl)-1-ethyl-2-oxo-1,2- dihydropyridine-3- carboxylic acid | MS(ES): 523 (M + 1) for C₂₂H₁₅ClF₄N₆O₃ ¹H NMR (300 MHz, DMSO-d₆) δ ppm 1.32 (t, 3 H) 4.15 (q, J = 7.10 Hz, 2 H) 7.09 (d, J = 2.64 Hz, 1 H) 7.43 (t, J = 9.04 Hz, 1 H) 7.59-7.82 (m, 1 H) 7.92- 8.23 (m, 2 H) 8.38 (d, J = 2.64 Hz, 1 H) 8.61 (d, J = 1.70 Hz, 1 H) 8.78 (s, 1 H) 10.41 (s, 1 H) 14.55 (s, 1 H) | Ethyl 5-(2-(3- chloro-4- fluorophenyl- amino)-4-(3- (trifluoromethyl)- 1H-pyrazol-1- yl)pyrimidin-5- yl)-1-ethyl-2- oxo-1,2- dihydropyridine- 3-carboxylate Example 1044 |
| Example 1086 | 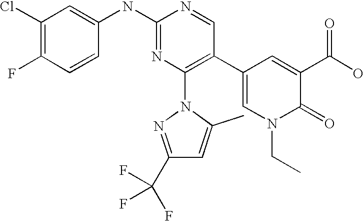 5-(2-(3-Chloro-4- fluorophenylamino)-4-(5- methyl-3- (trifluoromethyl)-1H- pyrazol-1-yl)pyrimidin- 5-yl)-1-ethyl-2-oxo-1,2- dihydropyridine-3- carboxylic acid | MS(ES): 537 (M + 1) for C₂₃H₁₇ClF₄N₆O₃ ¹H NMR (300 MHz, DMSO-d₆) δ ppm 1.28 (t, J = 7.16 Hz, 3 H) 2.48 (s, 3 H) 4.12 (q, J = 7.22 Hz, 2 H) 6.81 (s, 1 H) 7.42 (t, J = 9.14 Hz, 1 H) 7.64 (ddd, J = 9.04, 4.14, 2.64 Hz, 1 H) 7.80 (d, J = 2.64 Hz, 1 H) 8.05 (dd, J = 6.78, 2.64 Hz, 1 H) 8.30 (d, J = 2.64 Hz, 1 H) 8.92 (s, 1 H) 10.41 (s, 1 H) 14.42 (s, 1 H) | Ethyl 5-(2-(3- chloro-4- fluorophenyl- amino)-4-(5- methyl-3- (trifluoromethyl)- 1H-pyrazol-1- yl)pyrimidin-5- yl)-1-ethyl-2- oxo-1,2- dihydropyridine- 3-carboxylate Example 1045 |
| Example 1087 | 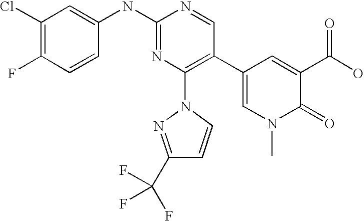 5-(2-(3-Chloro-4- fluorophenylamino)-4-(3- (trifluoromethyl)-1H- pyrazol-1-yl)pyrimidin-5- yl)-1-methyl-2-oxo-1,2- dihydropyridine-3- carboxylic acid | MS(ES): 509 (M + 1) for C₂₁H₁₃ClF₄N₆O₃ ¹H NMR (300 MHz, DMSO-d₆) δ ppm 3.70 (s, 3 H) 7.09 (d, J = 2.83 Hz, 1 H) 7.43 (t, J = 9.14 Hz, 1 H) 7.72 (ddd, J = 9.14, 4.24, 2.64 Hz, 1 H) 7.92-8.17 (m, 2 H) 8.42 (d, J = 2.64 Hz, 1 H) 8.52-8.68 (m, 1 H) 8.75 (s, 1 H) 10.40 (s, 1 H) 14.49 (s, 1 H) | Methyl 5-(2-(3- chloro-4- fluorophenyl- amino)-4-(3- (trifluoromethyl)- 1H-pyrazol-1- yl)pyrimidin-5- yl)-1-methyl-2- oxo-1,2- dihydrpyridine- 3-carboxylate Example 1046 |
| Example 1088 | 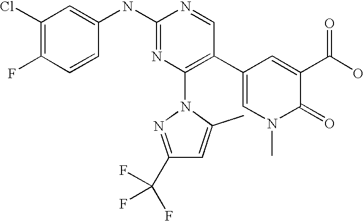 5-(2-(3-chloro-4- fluorophenylamino)-4-(5- methyl-3- (trifluoromethyl)-1H- pyrazol-1-yl)pyrimidin-5- yl)-1-methyl-2-oxo-1,2- dihydrpyridine-3- carboxylic acid | MS(ES): 523 (M + 1) for C₂₂H₁₅ClF₄N₆O₃ ¹H NMR (300 MHz, DMSO-d₆) δ ppm 2.49 (s, 3 H) 3.67 (s, 3 H) 6.82 (s, 1 H) 7.42 (t, J = 9.04 Hz, 1 H) 7.49-7.84 (m, 2 H) 8.04 (dd, J = 6.78, 2.45 Hz, 1 H) 8.41 (d, J = 2.45 Hz, 1 H) 8.89 (s, 1 H) 10.40 (s, 1 H) 14.36 (s, 1 H) | Methyl 5-(2-(3- chloro-4- fluorophenyl- amino)-4-(5- methyl-3- (trifluoromethyl)- 1H-pyrazol-1- yl)pyrimidin-5- yl)-1-methyl-2- oxo-1,2- dihydropyridine- 3-carboxylate Example 1047 |
| Example 1089 | 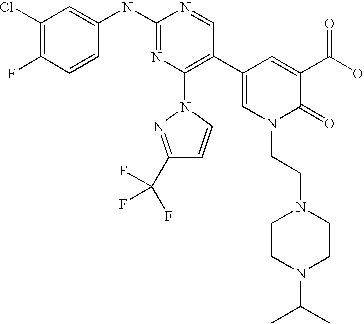 5-(2-(3-chloro-4- fluorophenylamino)-4-(3- (trifluoromethyl)-1H- pyrazol-1-yl)pyrimidin-5- yl)-1-(2-(4- isopropylpiperazin-1- yl)ethyl)-2-oxo-1,2- dihydropyridine-3- carboxylic acid | MS(ES): 649 M + 1) for C₂₉H₁₉ClF₄N₈O₃ ¹H NMR (300 MHz, DMSO-d₆) δ ppm 1.19 (d, J = 6.59 Hz, 6 H) 2.31-2.46 (m, 2 H) 2.74-2.98 (m, 4 H) 3.07 (d, J = 11.68 Hz, 2 H) 3.20-3.58 (m, 3 H) 4.24 (t, J = 5.84 Hz, 2 H) 7.11 (d, J = 2.83 Hz, 1 H) 7.43 (t, J = 9.04 Hz, 1 H) 7.71 (ddd, J = 9.09, 4.19, 2.73 Hz, 1 H) 7.97-8.17 (m, 2 H) 8.34 (d, J = 2.45 Hz, 1 H) 8.54-8.67 (m, 1 H) 8.73 (s, 1 H) 9.10 (br. s., 1 H) 10.41 (s, 1 H) | Methyl 5-(2-(3- chloro-4- fluorophenyl- amino)-4-(3- (trifluoromethyl)- 1H-pyrazol-1- yl)pyrimidin-5- yl)-1-(2-(4- isopropyl- piperazin-1- yl)ethyl-2- oxo-1,2- dihydropyridine- 3-carboxylate Example 1050 |
| Example 1090 | 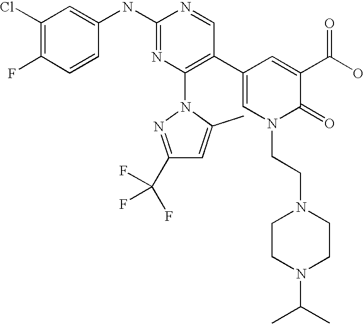 5-(2-(3-chloro-4- fluorophenylamino)-4-(5- methyl-3- (trifluoromethyl)-1H- pyrazol-1-yl)pyrimidin-5- yl)-1-(2-(4- isopropylpiperazin-1- yl)ethyl)-2-oxo-1,2- dihydropyridine-3- carboxylic acid | MS(ES): 663 (M + 1) for C₃₀H₃₁ClF₄N₈O₃ ¹H NMR (300 MHz, DMSO-d₆) δ ppm 1.20 (d, J = 6.59 Hz, 6 H) 2.35-2.49 (m, 5 H) 2.65-2.97 (m, 4 H) 3.06 (d, J = 12.62 Hz, 2 H) 3.25-3.53 (m, 3 H) 4.23 (t, J = 5.65 Hz, 2 H) 6.82 (s, 1 H) 7.43 (t, J = 9.14 Hz, 1 H) 7.55-7.71 (m, 2 H) 8.08 (dd, J = 6.78, 2.64 Hz, 1 H) 8.36 (d, J = 2.64 Hz, 1 H) 8.87 (s, 1 H) 9.11 (br. s., 1 H) 10.42 (s, 1 H) | Methyl 5-(2-(3- chloro-4- fluoprophenyl- amino)-4-(5- methyl- 3- (trifluoromethyl)- 1H-pyrazol-1- yl)pyrimidin-5- yl)-1-(2-(4- isopropyl- piperazin-1-yl)ethyl)- 2-oxo-1,2- dihydropyridine- 3-carboxylate Example 1018 |
| Example 1091 | 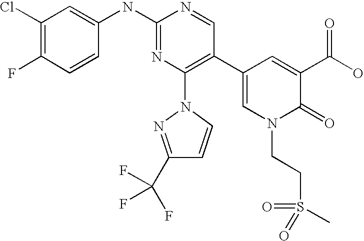 5-(2-(3-Chloro-4- fluorophenylamino)-4-(3- (trifluoromethyl)-1H- pyrazol-1-yl)pyrimidin-5- yl)-1-(2- (methylsulfonyl)ethyl)-2- oxo-1,2-dihydropyridine- 3-carboxylic acid | MS(ES): 601 (M + 1) for C₂₃H₁₇ClF₄N₆O₅S ¹H NMR (300 MHz, DMSO-d₆) δ ppm 3.10 (s, 3 H) 3.71 (t, J = 6.97 Hz, 2 H) 4.56 (t, J = 6.78 Hz, 2 H) 7.10 (d, J = 2.64 Hz, 1 H) 7.43 (t, J = 9.14 Hz, 1 H) 7.63-7.81 (m, 1 H) 7.99- 8.16 (m, 2 H) 8.44 (d, J = 2.64 Hz, 1 H) 8.54-8.66 (m, 1 H) 8.72 (s, 1 H) 10.42 (s, 1 H) | Methyl 5-(2-(3- chloro-4- fluorophenyl- amino)-4-(3- (trifluoromethyl)- 1H-pyrazol-1- yl)pyrimidin-5- yl)-1-(2- (methylsulfonyl) ethyl)-2-oxo- 1,2- dihydropyridine- 3-carboxylate Example 1020 |
| Example 1092 | 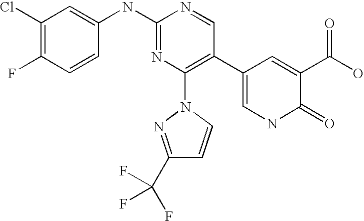 5-(2-(3-Chloro-4- fluorophenylamino)-4-(3- (trifluoromethyl)-1H- pyrazol-1-yl)pyrimidin-5- yl)-2-oxo-1,2- dihydropyridine-3- carboxylic acid | MS(ES): 495 (M + 1) for C₂₀H₁₁ClF₄N₆O₃ ¹H NMR (300 MHz, DMSO-d₆) δ ppm 7.07 (d, 1 H) 7.42 (t, J = 9.14 Hz, 1 H) 7.62-7.83 (m, 1 H) 7.90-8.20 (m, 3 H) 8.59 (s, 1 H) 8.74 (s, 1 H) 10.37 (s, 1 H) 14.64 (br. s., 1 H) | Methyl 5-(2-(3- chloro-4- fluorophenyl- amino)-4-(3- (trifluoromethyl)- 1H-pyrazol-1- yl)pyrimidin-5- yl)-1-(2- (methylsulfonyl) ethyl)-2-oxo- 1,2- dihydropyridine- 3-carboxylate Example 1020 |
| Example 1093 | 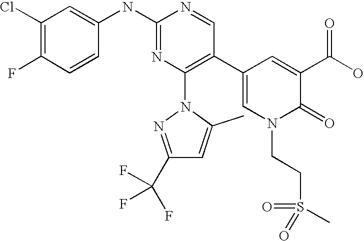 5-(2-(3-Chloro-4- fluorophenylamino)-4-(5- methyl-3- (trifluoromethyl)-1H- pyrazol-1-yl)pyrimidin-5- yl)-1-(2- (methylsulfonyl)ethyl)-2- oxo-1,2-dihydropyridine- 3-carboxylic acid | MS(ES): 615 (M + 1) for C₂₄H₁₉ClF₄N₆O₅S ¹H NMR (300 MHz, DMSO-d₆) δ ppm 2.46 (s, 3 H) 3.10 (s, 3 H) 3.69 (t, J = 6.69 Hz, 2 H) 4.54 (t, J = 6.69 Hz, 2 H) 6.81 (s, 1 H) 7.42 (t, J = 9.04 Hz, 1 H) 7.49-7.77 (m, 2 H) 8.05 (dd, J = 6.59, 2.26 Hz, 1 H) 8.48 (d, J = 2.45 Hz, 1 H) 8.87 (s, 1 H) 10.44 (s, 1 H) | Methyl 5-(2-(3- chloro-4- fluorophenyl- amino)-4-(5- methyl-3- (trifluoromethyl)- 1H-pyrazol-1- yl)pyrimidin-5- yl)-1-(2- (methylsulfonyl) ethyl)-2-oxo- 1,2- dihydropyridine- 3-carboxylate Example 1021 |
| Example 1094 | 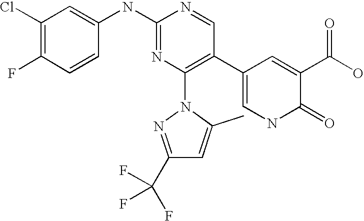 5-(2-(3-Chloro-4- fluorophenylamino)-4-(5- methyl-3- (trifluoromethyl)-1H- pyrazol-1-yl)pyrimidin-5- yl)-2-oxo-1,2- dihydropyridine-3- carboxylic acid | MS(ES): 509 (M + 1) for C₂₁H₁₃ClF₄N₆O₃ ¹H NMR (300 MHz, DMSO-d₆) δ ppm 2.49 (s, 3 H) 6.80 (s, 1 H) 7.42 (t, J = 9.14 Hz, 1 H) 7.64 (ddd, J = 9.04, 4.24, 2.73 Hz, 1 H) 7.78 (d, J = 2.64 Hz, 1 H) 7.87-8.16 (m, 2 H) 8.88 (s, 1 H) 10.37 (s, 1 H) 13.53 (br. s., 1 H) | Methyl 5-(2-(3- chloro-4- fluorophenyl- amino)-4-(5- methyl-3- (trifluoromethyl)- 1H-pyrazol-1- yl)pyrimidin-5- yl)-1-(2- (methylsulfonyl) ethyl)-2-oxo- 1,2- dihydropyridine- 3-carboxylate Example 1021 |
| Example 1095 | 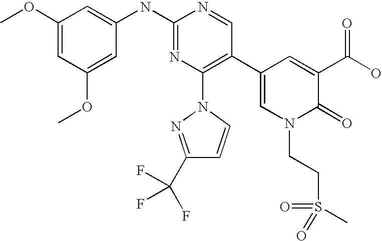 5-(2-(3,5- dimethoxyphenylamino)- 4-(3-(trifluoromethyl)-1H- pyrazol-1-yl)pyrimidin-5- yl)-1-(2- (methylsulfonyl)ethyl)-2- oxo-1,2-dihydropyridine- 3-carboxylic acid | MS(ES): 609 (M + 1) for C₂₅H₂₃F₃N₆O₇S ¹H NMR (300 MHz, DMSO-d₆) δ ppm 3.10 (s, 3 H) 3.71 (t, J = 7.06 Hz, 2 H) 3.75 (s, 6 H) 4.56 (t, J = 6.88 Hz, 2 H) 6.22 (t, J = 2.17 Hz, 1 H) 6.99-7.17 (m, 3 H) 8.05 (d, J = 2.64 Hz, 1 H) 8.45 (d, J = 2.64 Hz, 1 H) 8.58 (d, J = 1.70 Hz, 1 H) 8.70 (s, 1 H) 10.19 (s, 1 H) | Methyl 5-(2- (3,5- dimethoxyphenyl- amino)-4-(3- (trifluoromethyl)- 1H-pyrazol-1- yl)pyrimidin-5- yl)-1-(2- (methylsulfonyl) ethyl)-2-oxo- 1,2- dihydropyridine- 3-carboxylate Example 1022 |
| Example 1096 | 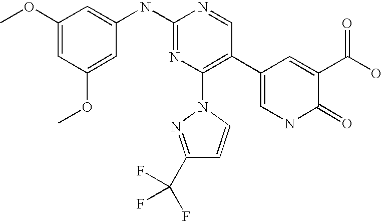 5-(2-(3,5- Dimethoxyphenylamino)- 4-(3-(trifluoromethyl)-1H- pyrazol-1-yl)pyrimidin-5- yl)-2-oxo-1,2- dihydropyridine-3- carboxylic acid | MS(ES): 503 (M + 1) for C₂₂H₁₇F₃N₆O₅ ¹H NMR (300 MHz, DMSO-d₆) δ ppm 3.75 (s, 6 H) 6.21 (t, J = 2.17 Hz, 1 H) 6.93-7.20 (m, 3 H) 8.02 (br. s., 1 H) 8.10 (d, J = 2.64 Hz, 1 H) 8.47-8.63 (m, 1 H) 8.72 (s, 1 H) 10.13 (s, 1 H) 13.48 (br. s., 1 H) | Methyl 5-(2- (3,5- dimethoxyphenyl- amino)-4-(3- (trifluoromethyl)- 1H-pyrazol-1- yl)pyrimidin-5- yl)-1-(2- (methylsulfonyl) ethyl)-2-oxo- 1,2- dihydropyridine- 3-carboxylate Example 1022 |
| Example 1097 | 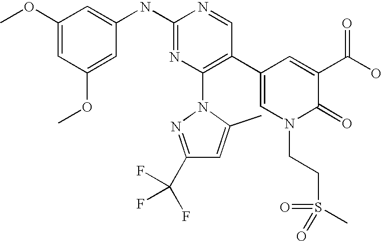 5-(2-(3,5- Dimethoxyphenylamino)- 4-(5-methyl-3- (trifluoromethyl)-1H- pyrazol-1-yl)pyrimidin-5- yl)-1-(2- (methylsulfonyl)ethyl)-2- oxo-1,2-dihydropyridine- 3-carboxylic acid | MS(ES): 623 (M + 1) for C₂₆H₂₅F₃N₆O₇S ¹H NMR (300 MHz, DMSO-d₆) δ ppm 2.44 (s, 3 H) 3.10 (s, 3 H) 3.58- 3.83 (m, 8 H) 4.54 (t, J = 6.78 Hz, 2 H) 6.22 (t, J = 2.17 Hz, 1 H) 6.80 (s, 1 H) 6.91-7.13 (m, 2 H) 7.57 (d, J = 2.64 Hz, 1 H) 8.48 (d, J = 2.64 Hz, 1 H) 8.85 (s, 1 H) 10.20 (s, 1 H) | Methyl 5-(2- (3,5- dimethoxyphenyl- amino)-4-(5- methyl-3- (trifluoromethyl)- 1H-pyrazol-1- yl)pyrimidin-5- yl)-1-(2- (methylsulfonyl) ethyl)-2-oxo- 1,2- dihydr5opyridine- 3-carboxylate Example 1023 |
| Example 1098 | 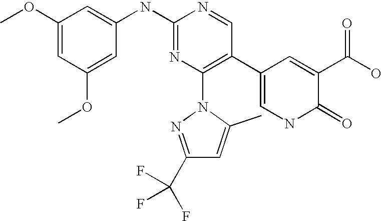 5-(2-(3,5- Dimethoxyphenylamino)- 4-(5-methyl-3- (trifluoromethyl)-1H- pyrazol-1-yl)pyrimidin-5- yl)-2-oxo-1,2- dihydropyridine-3- carboxylic acid | MS(ES): 517 (M + 1) for C₂₃H₁₉F₃N₆O₅ ¹H NMR (300 MHz, DMSO-d₆) δ ppm 2.43- 2.49 (m, 3 H) 3.73 (s, 6 H) 6.22 (t, J = 2.17 Hz, 1 H) 6.78 (s, 1 H) 6.92-7.14 (m, 2 H) 7.79 (d, J = 2.64 Hz, 1 H) 7.96 (d, J = 1.13 Hz, 1 H) 8.86 (s, 1 H) 10.13 (s, 1 H) 13.51 (br. s., 1 H) | Methyl 5-(2- (3,5- dimethoxyphenyl- amino)-4-(5- methyl-3- (trifluoromethyl)- 1H-pyrazol-1- yl)pyrimidin-5- yl)-1-(2- (methylsulfonyl) ethyl)-2-oxo- 1,2- dihydropyridine- 3-carboxylate Example 1023 |

### Example 1099

### 2-(3-(2-(3-chloro-4-fluorophenylamino)-4-(3-(trifluoromethyl)-1H-pyrazol-1-yl)pyrimidin-5yl)phenylsulfonyl)acetic acid

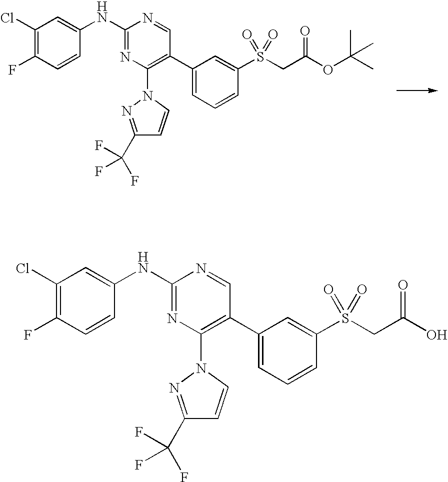

tert-butyl 2-(3-(2-(3-chloro-4-fluorophenylamino)-4-(3-(trifluoromethyl)-1H-pyrazol-1-yl)pyrimidin-5-yl)phenylsulfonyl)acetate Example 1003 (164 mg, 0.27 mmol) was dissolved in THF (2 mL), cooled to 0° C., treated with trifluoroacetic acid (2 mL), and allowed to stir at room temperature for 2 days. The solvent was removed at reduced pressure and the residue was purified by reverse phase preparative HPLC (C18: 45-95% ACN in H₂O containing 0.1% TFA) to afford the desired product (120 mg).

MS(ES): 556 (M+1) for C₂₂H₁₄ClF₄N₅O₄S

¹H NMR (300 MHz, DMSO-d₆) δ ppm 4.42 (s, 2H) 7.00 (d, J=2.64 Hz, 1H) 7.41 (t, J=9.14 Hz, 1H) 7.47-7.57 (m, 1H) 7.63 (t, J=7.72 Hz, 1H) 7.68-7.81 (m, 2H) 7.87 (d, J=7.91 Hz, 1H) 8.11 (dd, J=6.69, 2.54 Hz, 1H) 8.42 (d, J=1.51 Hz, 1H) 8.82 (s, 1H) 10.45 (s, 1H)

The following examples were prepared using the general HATU coupling method described for Example 360 using the starting materials (SM) indicated.

| Ex | Compound | Data | SM |
|---|---|---|---|
| Example 1100 | 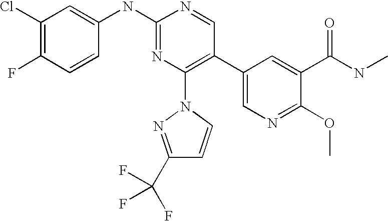 5-(2-(3-chloro-4- fluorophenylamino)-4- (3-(trifluoromethyl)-1H- pyrazol-1-yl)pyrimidin- 5-yl)-2-methoxy-N- methylnicotinamide | MS(ES): 522 (M + 1) for C₂₂H₁₆ClF₄N₇O₂ ¹H NMR (300 MHz, DMSO-d₆) δ ppm 2.79 (d, 3 H), 3.99 (s, 3 H) 7.03 (d, J = 2.64 Hz, 1 H) 7.42 (t, J = 9.14 Hz, 1 H) 7.72 (ddd, J = 8.90, 4.10, 2.64 Hz, 1 H) 7.91 (d, J = 2.45 Hz, 1 H) 8.09 (dd, J= 6.78, 2.64 Hz, 1 H) 8.16 (d, J = 2.45 Hz, 1 H) 8.19- 8.35 (m, 1 H) 8.47 (s, 1 H) 8.78 (s, 1 H) 10.42 (s, 1 H) | Methylamine and 5-(2-(3-chloro- 4- fluorophenylamino)- 4-(3- (trifluoro- methyl)-1H- pyrazol-1- yl)pyrimidin- 5-yl)-2- methoxynico- tinic acid Example 1066 |
| Example 1101 | 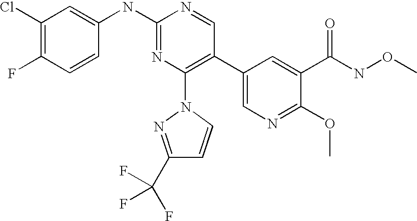 5-(2-(3-chloro-4- fluorophenylamino)-4- (3-(trifluoromethyl)-1H- pyrazol-1-yl)pyrimidin- 5-yl)-N,2- dimethoxynicotinamide | MS(ES): 538 (M + 1) for C₂₂H₁₆ClF₄N₇O₃ ¹H NMR (300 MHz, DMSO-d₆) δ ppm 3.68 (s, 3 H) 3.96 (s, 3 H) 7.04 (d, J = 2.64 Hz, 1 H) 7.42 (t, J = 9.04 Hz, 1 H) 7.64-7.79 (m, 1 H) 7.83 (d, J = 2.64 Hz, 1 H) 8.09 (dd, J = 6.69, 2.73 Hz, 1 H) 8.17 (d, J = 2.45 Hz, 1 H) 8.48 (s, 1 H) 8.79 (s, 1 H) 10.42 (s, 1 H) 11.31 (s, 1 H) | O- methylhydroxyl- amine hydrochloride and 5-(2-(3-chloro- 4- fluorophenylamino)- 4-(3- (trifluoromethyl)- 1H-pyrazol- 1- yl)pyrimidin- 5-yl)-2- methoxynico- tinic acid Example 1066 |
| Example 1102 | 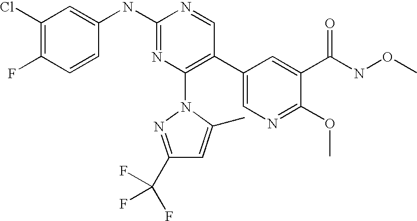 5-(2-(3-chloro-4- fluorophenylamino)-4- (5-methyl-3- (trifluoromethyl)-1H- pyrazol-1-yl)pyrimidin- 5-yl)-N,2- dimethoxynicotinamide | MS(ES): 552 (M + 1) for C₂₃H₁₈ClF₄N₇O₃ ¹H NMR (300 MHz, DMSO-d₆) δ ppm 2.33 (s, 3 H) 3.67 (s, 3 H) 3.92 (s, 3 H) 6.76 (s, 1 H), 7.42 (t, J = 9.14 Hz, 1 H) 7.55-7.77 (m, 2 H) 7.97-8.15 (m, 2 H) 8.95 (s, 1 H) 10.45 (s, 1 H) 11.30 (s, 1 H) | O- methylhydroxyl- amine hydrochloride and 5-(2-(3- chloro-4- fluorophenylamino)- 4-(5- methyl-3- (trifluoromethyl)- 1H- pyrazol-1- yl)pyrimidin- 5-yl)-2- methoxynico- tinic acid Example 1067 |
| Example 1103 | 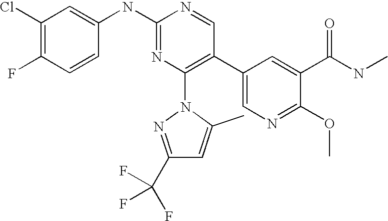 5-(2-(3-chloro-4- fluorophenylamino)-4- (5-methyl-3- (trifluoromethyl)-1H- pyrazol-1-yl)pyrimidin- 5-yl)-2-methoxy-N- methylnicotinamide | MS(ES): 536 (M + 1) for C₂₃H₁₈ClF₄N₇O₂ ¹H NMR (300 MHz, DMSO-d₆) δ ppm 2.34 (s, 3 H) 2.78 (d, J = 4.71 Hz, 3 H) 3.96 (s, 3 H) 6.76 (s, 1 H) 7.42 (t, J = 9.04 Hz, 1 H) 7.58-7.72 (m, 1 H) 7.76 (d, J = 2.64 Hz, 1 H) 7.99-8.14 (m, 2 H) 8.14-8.27 (m, 1 H) 8.83-9.00 (m, 1 H) 10.44 (s, 1 H) | Methylamine and 5-(2-(3- chloro-4- fluorophenyl amino)-4-(5- methyl-3- (trifluoromethyl)- 1H- pyrazol-1- yl)pyrimidin- 5-yl)-2- methoxynico- tinic acid Example 1067 |
| Example 1104 | 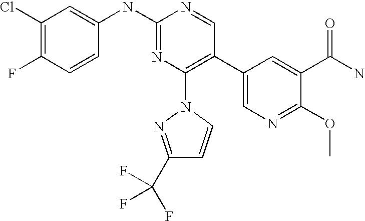 5-(2-(3-chloro-4- fluorophenylamino)-4- (3-(trifluoromethyl)-1H- pyrazol-1-yl)pyrimidin- 5-yl)-2- methoxynicotinamide | MS(ES): 508 (M + 1) for C₂₁H₁₄ClF₄N₇O₂ ¹H NMR (300 MHz, DMSO-d₆) δ ppm 4.00 (s, 3 H) 7.03 (d, J = 2.83 Hz, 1 H) 7.42 (t, J = 9.14 Hz, 1 H) 7.61-7.82 (m, 3 H) 7.93 (d, J = 2.45 Hz, 1 H) 8.09 (dd, J = 6.78, 2.64 Hz, 1 H) 8.18 (d, J = 2.45 Hz, 1 H) 8.48 (d, J = 1.51 Hz, 1 H) 8.79 (s, 1 H) 10.41 (s, 1 H) | Ammonia and 5-(2-(3-chloro- 4- fluorophenyl- amino)-4-(3- (trifluoromethyl)- 1H-pyrazol- 1- yl)pyrimidin- 5-yl)-2- methoxynico- tinic acid Example 1066 |
| Example 1105 | 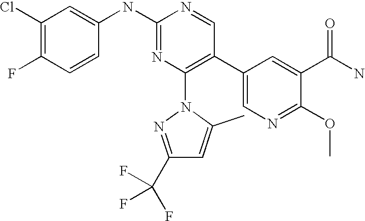 5-(2-(3-chloro-4- fluorophenylamino)-4- (5-methyl-3- (trifluoromethyl)-1H- pyrazol-1-yl)pyrimidin- 5-yl)-2- methoxynicotinamide | MS(ES): 522 (M + 1) for C₂₂H₁₆ClF₄N₇O₂ ¹H NMR (300 MHz, DMSO-d₆) δ ppm 2.34 (s, 3 H) 3.96 (s, 3 H) 6.75 (s, 1 H) 7.42 (t, J = 9.04 Hz, 1 H) 7.56-7.74 (m, 3 H) 7.78 (d, J = 2.64 Hz, 1 H) 7.95-8.16 (m, 2 H) 8.94 (s, 1 H) 10.44 (s, 1 H) | Ammonia and 5-(2-(3- chloro-4- fluorophenyl amino)-4-(5- methyl-3- (trifluoro- methyl)-1H- pyrazol-1- yl)pyrimidin- 5-yl)-2- methoxynico- tinic acid Example 1067 |
| Example 1106 | 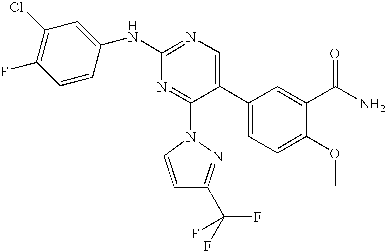 5-(2-(3-chloro-4- fluorophenylamino)-4- (3-(trifluoromethyl)-1H- pyrazol-1-yl)pyrimidin- 5-yl)-2- methoxybenzamide | MS(ES): 507 (M + 1) for C₂₂H₁₅ClF₄N₆O₂ 1H NMR (300 MHz, DMSO-d₆) δ ppm 3.90 (s, 3 H) 6.88-7.01 (m, 1 H) 7.08-7.19 (m, 1 H) 7.20-7.31 (m, 1 H) 7.33-7.45 (m, 1 H) 7.46-7.55 (m, 1 H) 7.62 (d, J = 2.26 Hz, 2 H) 7.65- 7.76 (m, 1 H) 8.06-8.18 (m, 1 H) 8.23-8.33 (m, 1 H) 8.75 (s, 1 H) 10.36 (s, 1 H) | Ammonia and 5-(2-(3- chloro-4- fluorophenyl amino)-4-(3- (trifluoro- methyl)-1H- pyrazol-1- yl)pyrimidin- 5-yl)-2- methoxy- benzoic acid Example 1074 |

The following examples were prepared using the general method described above for Example 1 using Intermediate 436 and the starting material (SM) indicated

| Ex | Compound | Data | SM |
|---|---|---|---|
| Example 1107 | 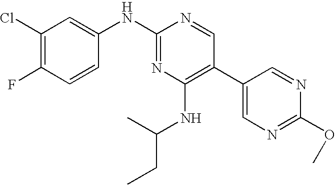 N′-butan-2-yl-N-(3- chloro-4- fluorophenyl)-5-(2- methoxypyrimidin- 5-yl)pyrimidine- 2,4-diamine | MS(ES): 403.1 (M + H) for C19H20ClFN6O. 1H NMR (300 MHz, DMSO-D6) δ ppm 0.86 (t, J = 7.44 Hz, 3 H) 1.06- 1.25 (m, 3 H), 1.36-1.81 (m, 2 H), 3.98 (s, 3 H), 4.03-4.24 (m, 1 H), 7.35-7.54 (m, 2 H), 7.75 (s, 1 H), 7.84 (s, 1 H), 8.07 (dd, J = 6.88, 2.35 Hz, 1 H), 8.57 (s, 2 H), 10.23 (s, 1 H). | 2- methoxy- pyrimidin-5- ylboronic acid |
| Example 1108 | 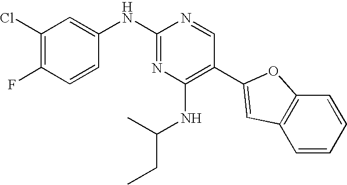 5-(1-benzofuran-2- yl)-N′-butan-2-yl- N-(3-chloro-4- fluorophenyl) pyrimidine-2,4-diamine | MS(ES): 411 (M + H) for C22H20ClFN4O. 1H NMR (300 MHz, DMSO-D6) δ ppm 0.95 (t, J = 7.35 Hz, 3 H) 1.25 (d, J = 6.59 Hz, 3 H) 1.44-1.93 (m, 2 H) 4.05-4.43 (m, 1 H) 7.17 (s, 1 H) 7.22-7.47 (m, 4 H) 7.49-7.58 (m, 1 H) 7.66 (dd, 2 H) 8.17 (dd, J = 6.78, 2.64 Hz, 1 H) 8.33 (s, 1 H) 10.04 (s, 1 H). | benzofuran- 2-ylboronic acid |
| Example 1109 | 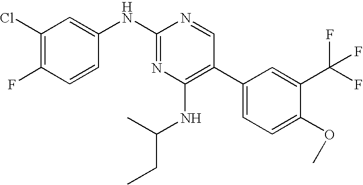 N′-butan-2-yl-N-(3- chloro-4- fluorophenyl)-5-[4- methoxy-3- (trifluoromethyl) phenyl]pyrimidine- 2,4-diamine | MS(ES): 468.9 (M + H) for C22H21ClF4N4O. 1H NMR (300 MHz, DMSO-D6) δ ppm 0.87 (t, J = 7.35 Hz, 3 H), 1.14 (d, J = 6.59 Hz, 3 H), 1.39-1.78 (m, 2 H), 3.95 (s, 3 H), 4.03-4.28 (m, 1 H), 7.28-7.71 (m, 6 H), 7.80 (s, 1 H), 8.06 (dd, J = 6.97, 2.07 Hz, 1 H), 10.23 (s, 1 H). | 4-methoxy-3- (trifluoro- methyl)phenyl- boronic acid |
| Example 1110 | 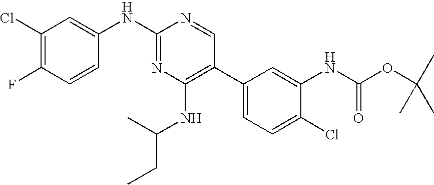 tert-butyl N-[5-[4- (butan-2-ylamino)- 2-[(3-chloro-4- fluorophenyl)amino] pyrimidin-5-yl]-2- chlorophenyl] carbamate | MS(ES): 520 (M + H) for C25H28Cl2FN5O2. 1H NMR (300 MHz, DMSO-D6) δ ppm 0.80 (t, J = 7.35 Hz, 3 H), 1.08 (d, J = 6.59 Hz, 3 H), 1.28-1.72 (m, 11 H), 4.05 (dd, 1 H), 7.07 (dd, J = 8.29, 2.07 Hz, 2 H), 7.30-7.55 (m, 3 H), 7.65 (d, J = 2.07 Hz, 1 H), 7.72 (s, 1 H), 8.01 (dd, J = 6.78, 2.45 Hz, 1 H), 8.77 (s, 1 H), 9.99 (s, 1 H). | 3-(tert- butoxycar- bonylamino)-4- chlorophenyl boronic acid |

The following examples were prepared using the general method described above for Example 1 using the starting materials (SM) indicated.

| Ex | Compound | Data | SM |
|---|---|---|---|
| Example 1111 | 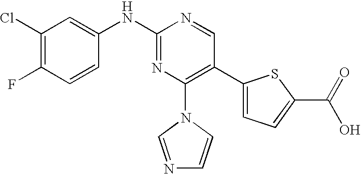 5-[2-[(3-chloro-4- fluorophenyl)- amino]-4-imidazol-1- ylpyrimidin-5- yl]thiophene-2- carboxylic acid | MS(ES): 414 (M − H) for C18H11ClFN5O2S. 1H NMR (300 MHz, DMSO-D6) δ ppm 7.10 (d, J = 3.96 Hz, 1 H), 7.25 (s, 1 H), 7.30-7.49 (m, 2 H), 7.56-7.70 (m, 2 H), 7.97 (dd, J = 6.69, 2.54 Hz, 1 H), 8.30 (s, 1 H), 8.83 (s, 1 H), 10.46 (s, 1 H), 13.0 (br. s, 1 H). | 5- boronothio- phene-2- carboxylic acid and Intermediate 437 |
| Example 1112 | 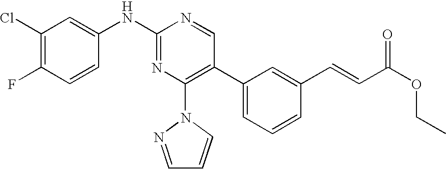 ethyl (E)-3-[3-[2- [(3-chloro-4- fluorophenyl) amino]-4-pyrazol-1- ylpyrimidin-5- yl]phenyl]prop-2- enoate | MS(ES): 464 (M + H) for C24H19ClFN5O2. 1H NMR (300 MHz, DMSO-D6) δ ppm 1.27 (t, 3 H), 4.18 (dd, 2 H), 6.55 (dd, J = 2.54, 1.60 Hz, 1 H), 6.62 (d, J = 16.20 Hz, 1 H), 7.15 (d, J = 7.72 Hz, 1 H), 7.28-7.51 (m, 2 H), 7.56-7.70 (m, 4 H), 7.69-7.81 (m, 1 H), 8.11 (dd, J = 6.78, 2.64 Hz, 1 H), 8.31 (d, J = 2.26 Hz, 1 H), 8.69 (s, 1 H), 10.29 (s, 1 H). | (E)-3-(3- ethoxy-3- oxoprop-1- enyl)phenyl- boronic acid and Intermediate 438 |
| Example 1113 | 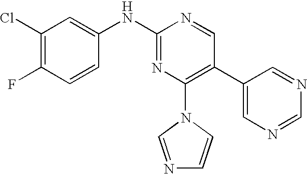 N-(3-chloro-4- fluorophenyl)-4- imidazol-1-yl-5- pyrimidin-5- ylpyrimidin-2- amine | MS(ES): 368 (M + H) for C17H11ClFN7. 1H NMR (300 MHz, DMSO-D6) δ ppm 7.23 (s, 1 H), 7.31-7.45 (m, 2 H), 7.61-7.73 (m, 1 H), 7.99 (dd, J = 6.69, 2.54 Hz, 1 H), 8.38 (s, 1 H), 8.65 (s, 2 H), 8.82 (s, 1 H), 9.13 (s, 1 H), 10.46 (s, 1 H). | pyrimidin-5- ylboronic acid and Intermediate 437 |
| Example 1114 | 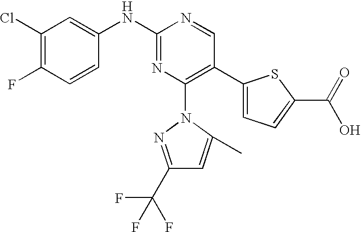 5-[2-[(3-chloro-4- fluorophenyl) amino]-4-[5-methyl- 3- (trifluoromethyl) pyrazol-1- yl]pyrimidin-5- yl]thiophene-2- carboxylic acid | MS(ES): 498 (M + H) for C20H12ClF4N5O2S. 1H NMR (300 MHz, DMSO-D6) δ ppm 2.20 (s, 3 H), 6.80 (s, 1 H), 7.10 (d, J = 3.96 Hz, 1 H), 7.37-7.46 (m, 2 H), 7.55-7.60 (m, 1 H), 7.90-8.03 (m, J = 3.96 Hz, 1 H), 9.10 (s, 1 H), 10.54 (s, 1 H), 13.08 (s, 1 H). | 5- boronothio- phene-2- carboxylic acid and Intermediate 113 |
| Example 1115 | 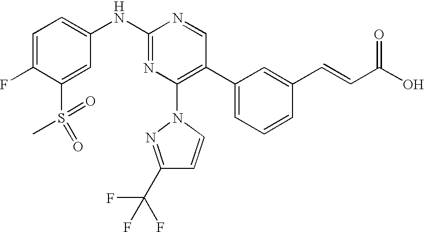 (E)-3-(3-(2-(4- fluoro-3- (methylsulfonyl) phenylamino)-4-(3- (trifluoromethyl)- 1H-pyrazol-1- yl)pyrimidin-5- yl)phenyl)acrylic acid | MS(ES): 546 (M − H) for C24H17F4N5O4S. 1H NMR (300 MHz, DMSO-D6) δ ppm 3.29 (s, 3 H), 6.41 (d, J = 16.01 Hz, 1 H), 6.94 (d, J = 2.64 Hz, 1 H), 7.11 (d, J = 7.91 Hz, 1 H), 7.31 (t, J = 7.72 Hz, 1 H), 7.38-7.53 (m, 3 H), 7.53-7.64 (m, J = 7.72 Hz, 1 H), 7.89- 8.07 (m, 1 H), 8.43 (s, 1 H), 8.50 (dd, J = 6.12, 2.73 Hz, 1 H), 8.76 (s, 1 H), 10.53 (s, 1 H), 12.31 (s, 1 H). | (E)-3-(3- boronophenyl) acrylic acid and Intermediate 445 |
| Example 1116 | 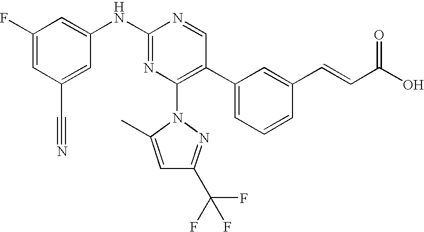 (E)-3-(3-(2-(3- cyano-5- fluorophenylamino)- 4-(5-methyl-3- (trifluoromethyl)- 1H-pyrazol-1- yl)pyrimidin-5- yl)phenyl)acrylic acid | MS(ES): 507 (M − H) for C25H16F4N6O2. 1H NMR (300 MHz, DMSO-D6) δ ppm 2.15 (s, 3 H), 6.38 (d, J = 16.01 Hz, 1 H), 6.67 (s, 1 H), 7.00 (d, J = 8.29 Hz, 1 H), 7.22-7.33 (m, 2 H), 7.35-7.50 (m, 2 H), 7.56 (d, J = 7.91 Hz, 1 H), 7.88-8.13 (m, 2 H), 9.00 (s, 1 H), 10.74 (s, 1 H), 12.34 (s, 1 H). | (E)-3-(3- boronophenyl) acrylic acid and Intermediate 446 |
| Example 1117 | 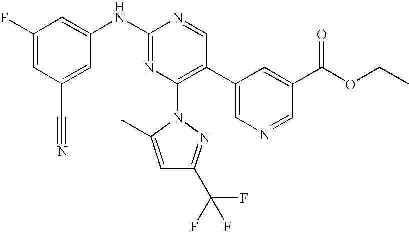 ethyl 5-(2-(3- cyano-5- fluorophenylamino)- 4-(5-methyl-3- (trifluoromethyl)- 1H-pyrazol-1- yl)pyrimidin-5- yl)nicotinate | MS(ES): 512 (M + H) for C24H17F4N7O2. 1H NMR (300 MHz, CHLOROFORM-D) δ ppm 1.42 (t, J = 7.06 Hz, 3 H), 2.46 (s, 3 H), 4.44 (q, J = 7.16 Hz, 2 H), 6.46 (s, 1 H), 7.00-7.23 (m, 1 H), 7.71 (s, 1 H), 7.75-7.90 (m, 2 H), 8.03 (t, J = 2.07 Hz, 1 H), 8.49 (d, J = 2.26 Hz, 1 H), 8.76 (s, 1 H), 9.20 (d, J = 1.88 Hz, 1 H) | ethyl 5- (4,4,5,5- tetramethyl- 1,3,2- dioxaborolan- 2-yl)nicotinate and Intermediate 446 |
| Example 1118 | 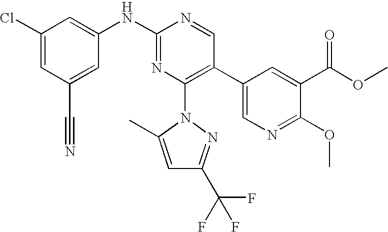 methyl 5-(2-(3- chloro-5- cyanophenylamino)- 4-(5-methyl-3- (trifluoromethyl)- 1H-pyrazol-1- yl)pyrimidin-5- yl)-2- methoxynicotinate | MS(ES): 544 (M + H) for C24H17ClF3N7O3. 1H NMR (300 MHz, DMSO-D6) δ ppm 2.35 (s, 3 H), 3.76 (s, 3 H), 3.92 (s, 3 H), 6.79 (s, 1 H), 7.53-7.75 (m, 2 H), 8.05-8.40 (m, 3 H), 9.05 (s, 1 H), 10.76 (s, 1 H). | Intermediate 447 and Intermediate 175 |
| Example 1119 | 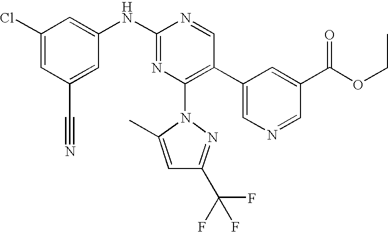 ethyl 5-(2-(3- chloro-5- cyanophenylamino)- 4-(5-methyl-3- (trifluoromethyl)- 1H-pyrazol-1- yl)pyrimidin-5- yl)nicotinate | MS(ES): 528 (M + H) for C24H17ClF3N7O2. 1H NMR (300 MHz, DMSO-D6) δ ppm 1.27-1.38 (t, J = 7.1 Hz, 3 H), 2.45 (s, 3 H), 4.25-4.38 (q, J = 7.10 Hz, 2 H), 6.81 (s, 1 H), 7.62-7.73 (m, 1 H), 7.84 (t, J = 2.07 Hz, 1 H), 8.27 (d, J = 1.32 Hz, 1 H), 8.21 (d, J = 1.98 Hz, 1 H), 8.63 (d, J = 2.26 Hz, 1 H), 9.01 (d, J = 2.07 Hz, 1 H), 9.10 (s, 1 H), 10.80 (s, 1 H). | Intermediate 447 and ethyl 5- (4,4,5,5- tetramethyl- 1,3,2- dioxaborolan- 2- yl)nicotinate |

The following examples were prepared using the general method described for example 214 using 1N sodium hydroxide, 1,4-dioxane and the starting material (SM) indicated.

| Ex | Compound | Data | SM |
|---|---|---|---|
| Example 1120 | 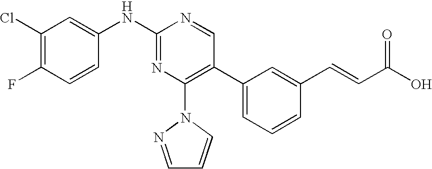 (E)-3-[3-[2-[(3- chloro-4- fluorophenyl) amino]-4-pyrazol-1- ylpyrimidin-5- yl]phenyl]prop-2- enoic acid | MS(ES): 436 (M + H) for C22H15ClFN5O2. 1H NMR (300 MHz, DMSO-D6) δ ppm 6.42-6.61 (m, 2 H), 7.15 (d, J = 7.91 Hz, 1 H), 7.30-7.48 (m, 2 H), 7.49-7.66 (m, 4 H), 7.73 (dd, 1 H), 8.11 (dd, J = 6.78, 2.64 Hz, 1 H), 8.31 (d, J = 2.26 Hz, 1 H), 8.69 (s, 1 H), 10.28 (s, 1 H), 12.43 (s, 1 H). | Example1112 |
| Example 1121 | 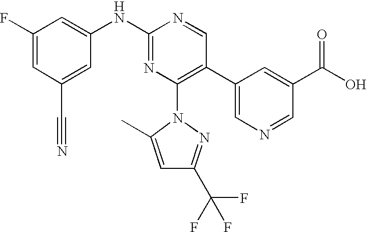 5-(2-(3-cyano-5- fluorophenylamino)- 4-(5-methyl-3- (trifluoromethyl)- 1H-pyrazol-1- yl)pyrimidin-5- yl)nicotinic acid | MS(ES): 484 (M + H) for C22H13F4N7O2. 1H NMR (300 MHz, DMSO-D6) δ ppm 2.44 (s, 3 H), 6.79 (s, 1 H), 7.42- 7.58 (m, 1 H), 7.86 (t, J = 2.17 Hz, 1 H), 7.95-8.13 (m, 2 H), 8.57 (d, J = 2.26 Hz, 1 H), 8.98 (d, J = 1.88 Hz, 1 H), 9.08 (s, 1 H), 10.82 (s, 1 H), 13.44 (s, 1 H). | Example 1117 |
| Example 1122 | 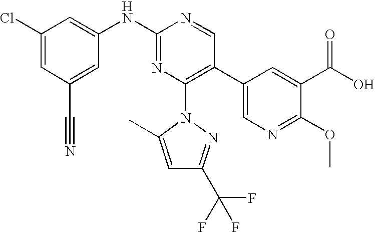 5-(2-(3-chloro-5- cyanophenylamino)- 4-(5-methyl-3- (trifluoromethyl)- 1H-pyrazol-1- yl)pyrimidin-5- yl)-2- methoxynicotinic acid | MS(ES): 529.8 (M + H) for C23H15ClF3N7O3. 1H NMR (300 MHz, DMSO-D6) δ ppm 2.36 (s, 3 H), 3.92 (s, 3 H), 6.78 (s, 1 H), 7.49-7.82 (m, 2 H, 8.04- 8.38 (m, 3 H), 9.04 (s, 1 H), 10.74 (s, 1 H), 12.94 (s, 1 H). | Example 1118 |
| Example 1123 | 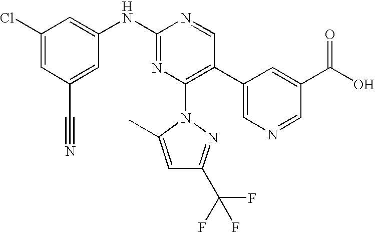 5-(2-(3-chloro-5- cyanophenylamino)- 4-(5-methyl-3- (trifluoromethyl)- 1H-pyrazol-1- yl)pyrimidin-5- yl)nicotinic acid | MS(ES): 500 (M + H) for C22H13ClF3N7O2. 1H NMR (300 MHz, DMSO-D6) δ ppm 2.45 (s, 3 H), 6.80 (s, 1 H), 7.68 (d, J = 1.51 Hz, 1 H), 7.86 (t, J = 2.07 Hz, 1 H), 8.08-8.30 (m, 2 H), 8.57 (d, J = 2.26 Hz, 1 H), 8.98 (d, J = 1.88 Hz, 1 H), 9.08 (s, 1 H), 10.78 (s, 1 H), 13.44 (s, 1 H). | Example 1119 |

The following examples were prepared using the general HATU coupling method described for Example 360 using Example 320 and the starting material indicated.

| Ex | Compound | Data | SM |
|---|---|---|---|
| Example 1124 | 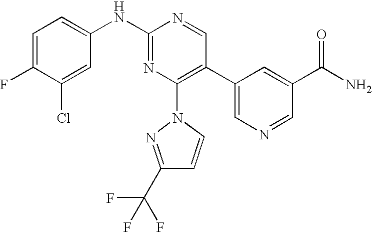 5-[2-[(3-chloro-4- fluorophenyl) amino]-4-[3- (trifluoromethyl) pyrazol-1- yl]pyrimidin-5- yl]pyridine-3- carboxamide | MS(ES): 478 (M + H) for C20H12ClF4N7O. 1H NMR (300 MHz, DMSO-D6) δ ppm 6.99 (d, J = 2.64 Hz, 1 H), 7.37 (t, J = 9.14 Hz, 1 H), 7.57 (s, 1 H), 7.62- 7.76 (m, 1 H), 7.94-8.16 (m, 3 H), 8.42 (d, J = 2.26 Hz, 1 H), 8.48 (d, J = 1.70 Hz, 1 H), 8.77 (s, 1 H), 8.91 (d, J = 2.07 Hz, 1 H), 10.41 (s, 1 H). | Ammonia in dioxane (0.5 M) |
| Example 1125 | 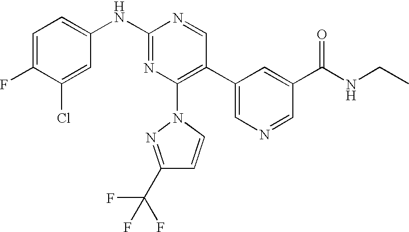 5-[2-[(3-chloro-4- fluorophenyl) amino]-4-[3- (trifluoromethyl) pyrazol-1- yl]pyrimidin-5- yl]-N- ethylpyridine-3- carboxamide | MS(ES): 504 (M − H) for C22H16ClF4N7O. 1H NMR (300 MHz, DMSO-D⁶) δ ppm 1.06 (t, J = 7.25 Hz, 3 H); 3.11- 3.41 (m, 2 H); 6.99 (d, J = 2.64 Hz, 1 H); 7.37 (t, J = 9.14 Hz, 1 H); 7.59- 7.76 (m, 1 H); 7.95-8.14 (m, 2 H); 8.41 (d, J = 2.07 Hz, 1 H); 8.48 (t, J= 1.51 Hz, 1 H); 8.61 (t, J = 5.46 Hz, 1 H); 8.77 (s, 1 H); 8.88 (d, J = 2.07 Hz, 1 H); 10.42 (s, 1 H). | ethylamine |

### Intermediate 1126: 5-(4-(3-(difluoromethyl)-5-methyl-1H-pyrazol-1-yl)-2-(3,5-dimethoxyphenylamino)pyrimidin-5-yl)nicotinic acid

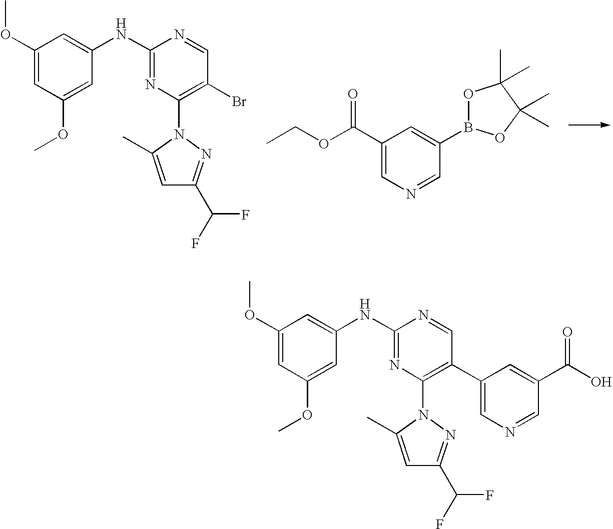

5-bromo-4-(3-(difluoromethyl)-5-methyl-1H-pyrazol-1-yl)-N-(3,5-dimethoxyphenyl)pyrimidin-2-amine Intermediate 449 (210 mg, 0.48 mmol), ethyl 5-(4,4,5,5-tetramethyl-1,3,2-dioxaborolan-2-yl)nicotinate (159 mg, 0.57 mmol), and PdCl2(dppf-CH2Cl2Adduct (117 mg, 0.14 mmol) were combined in acetonitrile (10 mL) to give a yellow suspension. Sodium carbonate (60.7 mg, 0.57 mmol) was added, followed by water (2.500 mL) and the mixture was degassed with argon then heated at 80° C. for 4 hours. Adsorption onto silica gel, followed by flash chromatography (0.5-10% methanol in dichloromethane) gave the intermediate ester compound (235 mg) which was hydrolyzed to the corresponding carboxylic acid without further characterization as follows: The ester was dissolved in Dioxane (5 ml), then 1N NaOH solution (0.690 ml, 0.69 mmol) was added and the mixture was allowed to stir at room temperature for 6 hours. The reaction mixture was neutralized with 1M HCl followed by purification by reverse phase chromatography (C18: 5-95% acetonitrile in water, 0.5% TFA) to give the title compound (89 mg). MS:ES+482.44 for C₂₃H₂₀F₂N₆O₄

1H NMR (300 MHz, DMSO-d6) δ ppm 2.41 (s, 3H) 3.73 (s, 6 H) 6.10-6.37 (m, 1H) 6.55 (s, 2H) 7.06 (s, 2H) 7.75-7.99 (m, 1H) 8.39-8.65 (m, 1H) 8.93 (br. s., 2H) 10.05-10.34 (m, 1H) 13.25-13.64 (m, 1H)

The compounds in the below table were prepared using this procedure and the specified starting materials.

| Ex | Compound | Data | SM |
|---|---|---|---|
| Example 1127 | 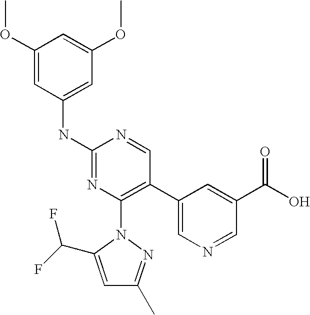 5-(4-(5-(difluoromethyl)-3- methyl-1H-pyrazol-1-yl)-2- (3,5- dimethoxyphenylamino) pyrimidin-5-yl)nicotinic acid | MS: ES+ 482.44 for C₂₃H₂₀F₂N₆O₄ 1H NMR (300 MHz, DMSO-d6) □ppm 1.98 (s, 3 H) 3.74 (s, 6 H) 6.23 (br. s., 1 H) 6.72 (s, 1 H) 7.01 (d, J = 1.32 Hz, 2 H) 7.96 (br. s., 2 H) 8.50 (br. s., 1 H) 8.79 (s, 1 H) 8.96 (br. s., 1 H) 9.97 (s, 1 H) 13.21-13.86 (m, 1 H) | ethyl 5-(4,4,5,5- tetramethyl-1,3,2- dioxaborolan-2- yl)nicotinate and Intermediate 450 |
| Example 1128 | 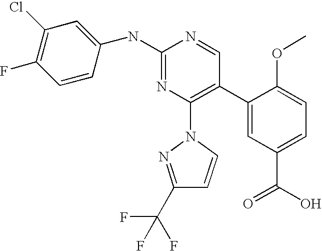 3-(2-(3-chloro-4- fluorophenylamino)-4-(3- (trifluoromethyl)-1H-pyrazol- 1-yl)pyrimidin-5-yl)-4- methoxybenzoic acid | MS: ES+ 508.82 for C₂₂H₁₄ClF₄N₅O₃ H NMR (300 MHz, DMSO-d6) d ppm 3.44 (br. s., 3 H) 6.97 (br. s., 2 H) 7.41 (br. s., 1 H), 7.73 (br. s., 1 H) 7.87 (br. s., 2 H) 8.10 (br. s., 1 H) 8.42 (br. s., 1 H) 8.67 (br. s., 1 H) 10.34 (br. s., 1 H) 12.35-13.05 (m, 1 H) | 2-methoxy-5- (methoxycarbonyl) phenylboronic acid And Intermediate 115 |

### Example 1129

### (E)-3-(3-(6-(3-chloro-4-fluorophenylamino)-4-morpholinopyridin-3-yl_phenyl)acrylic acid

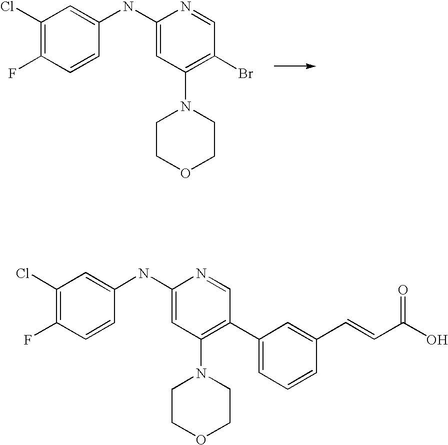

5-bromo-N-(3-chloro-4-fluorophenyl)-4-morpholinopyridin-2-amine Intermediate 454 (80 mg, 0.21 mmol), (E)-3-(3-boronophenyl)acrylic acid (55.6 mg, 0.29 mmol), and Pd2(dba)3 (18.95 mg, 0.02 mmol) were combined in acetonitrile (8 mL) to give a suspension. Dicyclohexyl(2′,4′, 6′-triisopropylbiphenyl-2-yl)phosphine (29.6 mg, 0.06 mmol) and Na₂CO₃ (43.9 mg, 0.41 mmol) were added followed by water (2.000 mL). The reaction was degassed with argon then heated at 80° C. for 30 minutes. Adsorption onto silica gel followed by purification by flash chromatography (3-25% methanol in dichloromethane) gave the title compound (52 mg). MS (Electrospray): 454.89 (MH⁺) for C₂₄H₂₁ClFN₃O₃

1H NMR (300 MHz, DMSO-d6) δ ppm 2.82 (br. s., 4H) 3.54 (br. s., 4H) 6.37 (s, 1H) 6.49-6.64 (m, 1H) 7.29 (s, 1H) 7.48 (d, J=7.35 Hz, 3H) 7.61 (s, 2H) 7.80-7.99 (m, 2H) 8.05-8.26 (m, 1H) 9.26 (s, 1H),12.22-12.74 (m, 1H)

### Example 1130

### 5-(2-(3-chloro-4-fluorophenylamino)-4-(5-methyl-3-(trifluoromethyl)-1H-pyrazol-1-yl)pyrimidin-5-yl)-2-(2-methoxyethoxy)nicotinic acid

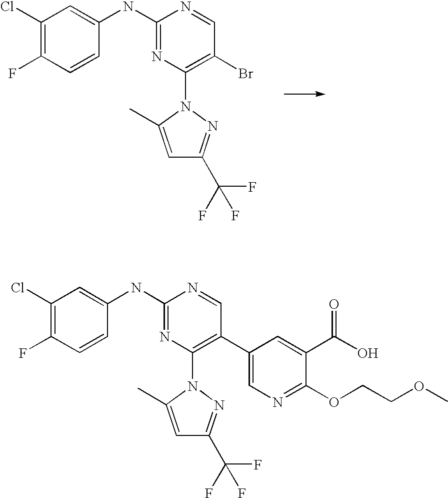

5-bromo-N-(3-chloro-4-fluorophenyl)-4-(5-methyl-3-(trifluoromethyl)-1H-pyrazol-1-yl)pyrimidin-2-amine Intermediate 113 (191 mg, 0.42 mmol), methyl 2-(2-methoxyethoxy)-5-(4,4,5,5-tetramethyl-1,3,2-dioxaborolan-2-yl)nicotinate, Intermediate 465 (200 mg, 0.59 mmol), Pd₂(dba)₃ (38 mg, 0.04 mmol) and dicyclohexyl(2′,4′,6′-triisopropylbiphenyl-2-yl)phosphine (60 mg, 0.13 mmol) were combined in acetonitrile (10 mL) to give a yellow suspension. Sodium carbonate (67 mg, 0.64 mmol) was added, followed by water (2.500 mL) and the mixture was degassed using argon then heated at 80° C. for 4 hours. Purification by flash chromatography (0.5-10% methanol in dichloromethane) gave the intermediate carboxylic ester (265 mg), which was hydrolyzed to the corresponding carboxylic acid as below.

MS (Electrospray): 581.92 (MH⁺) for C₂₅H₂₁ClF₄N₆O₄

The intermediate ester, methyl 5-(2-(3-chloro-4-fluorophenylamino)-4-(5-methyl-3-(trifluoromethyl)-1H-pyrazol-1-yl)pyrimidin-5-yl)-2-(2-methoxyethoxy)nicotinate (265mg, 0.45 mmol), was dissolved in THF (1 ml) and Dioxane (3 ml). 1N NaOH (1.12 ml, 1.12 mmol) was added and the mixture was allowed to stir at room temperature overnight. The reaction mixture was neutralized with 1M HCl then evaporated. The residue was purified by reverse phase chromatography (C18: 35-95% acetonitrile in water, 0.1% TFA) to give the title compound (60 mg). MS (Electrospray): 567 (MH⁺) for C₂₄H₁₉ClF₄N₆O₄

1H NMR (300 MHz, DMSO-d6) δ ppm 2.36 (s, 3H) 3.30 (s, 3H) 3.63-3.70 (m, 2H), 4.33-4.54 (m, 2H), 6.76 (s, 1H) 7.42 (t,J=9.14 Hz, 1H) 7.64 (d, J=2.45 Hz, 2H) 7.93-8.30 (m, 2H), 8.95 (s, 1H) 10.42 (s, 1H) 12.90 (s, 1H)

The Compounds in the below table were prepared using this procedure and the specified starting materials.

| | | Mass spectrum | |
|---|---|---|---|
| Compound | Structure | and ¹H NMR | SM |
| Example 1131 | 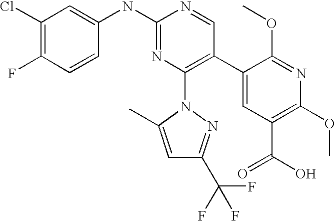 5-(2-(3-chloro-4-fluorophenylamino)-4- (5-methyl-3-(trifluoromethyl)-1H- pyrazol-1-yl)pyrimidin-5-yl)-2,6- dimethoxynicotinic acid | MS: ES+ 553.87 for C₂₃H₁₇ClF₄N₆O₄ 1H NMR (300 MHz, DMSO-d6) δ ppm 3.64 (s, 3 H) 3.94 (s, 3 H) 6.73- 6.78 (m, 1 H), 7.39- 7.49 (m, 1 H) 7.65- 7.74 (m, 1 H) 8.05 (s, 1 H) 8.06-8.13 (m, 1 H) 8.82 (s, 1 H) 10.29-10.43 (m, 1 H) 12.48- 12.68 (m, 1 H) | methyl 2,6- dimethoxy-5- (4,4,5,5- tetramethyl- 1,3,2- dioxaborolan-2- yl)nicotinate Intermediate 466 and Intermediate 113 |
| Example 1132 | 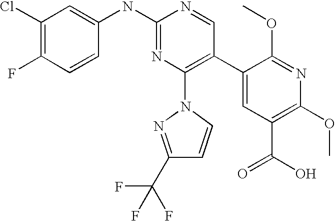 5-(2-(3-chloro-4-fluorophenylamino)-4- (3-(trifluoromethyl)-1H-pyrazol-1- yl)pyrimidin-5-yl)-2,6- dimethoxynicotinic acid | MS: ES+ 539.84 for C22H15ClF4N6O4 1H NMR (300 MHz, DMSO-d6) δ ppm 3.58 (s, 3 H) 3.93 (s, 3 H) 7.02 (d, J = 2.26 Hz, 1 H) 7.35-7.50 (m, 1H) 7.66-7.82 (m, 1 H) 8.01-8.13 (m, 2 H) 8.54 (br. s., 1 H) 8.69 (s, 1 H) 10.33 (s, 1 H) 12.24- 12.92 (m, 1) | methyl 2,6- dimethoxy-5- (4,4,5,5- tetramethyl- 1,3,2- dioxaborolan-2- yl)nicotinate Intermediate 466 and Intermediate 115 |
| Example 1133 | 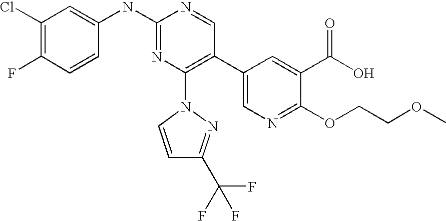 5-(2-(3-chloro-4-fluorophenylamino)-4- (3-(trifluoromethyl)-1H-pyrazol-1- yl)pyrimidin-5-yl)-2-(2- methoxyethoxy)nicotinic acid | 552.87 C₂₃H₁₇ClF₄O₄ 1H NMR (300 MHz, DMF) δ ppm 3.31 (s, 3 H) 3.62- 3.75 (m, 2 H) 4.42- 4.54 (m, 2 H) 7.03 (d, J = 2.64 Hz, 1 H) 7.42 (s, 1 H) 7.66- 7.78 (m, 1 H) 7.84 (d, J = 2.45 Hz, 1 H) 8.00-8.13 (m, 1 H) 8.20 (d, J = 2.45 Hz, 1 H) 8.50 (d, J = 1.70 Hz, 1 H) 8.79 (s, 1 H) 10.38 (s, 1 H) 12.77-13.00 (m, 1 H) | methyl 2-(2- methoxyethoxy)- 5-(4,4,5,5- tetramethyl- 1,3,2- dioxaborolan-2- yl)nicotinate Intermediate 465 and Intermediate 115 |
| Example 1134 | 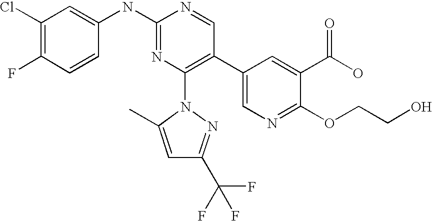 5-(2-(3-chloro-4-fluorophenylamino)-4- (5-methyl-3-(trifluoromethyl)-1H- pyrazol-1-yl)pyrimidin-5-yl)-2-(2- hydroxyethoxy)nicotinic acid | 552.88 C₂₃H₁₇ClF₄O₄ 1H NMR (300 MHz, DMSO-d6) δ ppm 2.34 (s, 3 H) 3.70 (t, J = 5.46 Hz, 2 H) 4.36 (t, J = 5.37 Hz, 2 H) 6.75 (s, 1 H) 7.42 (t, J = 9.04 Hz, 1 H) 7.90-8.27 (m, 2 H) 8.95 (s, 1 H) 10.41 (s, 1 H) | methyl 2-(2- hydroxyethoxy)- 5-(4,4,5,5- tetramethyl- 1,3,2- dioxaborolan-2- yl)nicotinate Intermediate 467 and Intermediate 113 |
| Example 1135 | 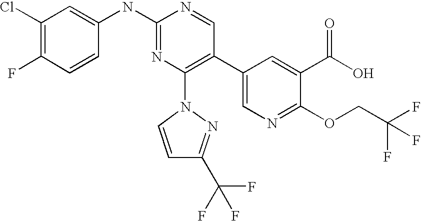 5-(2-(3-chloro-4-fluorophenylamino)-4- (3-(trifluoromethyl)-1H-pyrazol-1- yl)pyrimidin-5-yl)-2-(2,2,2- trifluoroethoxy)nicotinic acid | MS: ES+ 577.81 for C₂₂H₁₂ClF₇N₆O₃ 1H NMR (300 MHz, DMSO-d₆) δ ppm 5.11 (d, J = 8.85 Hz, 2 H) 7.04 (d, J = 2.64 Hz, 1 H) 7.42 (s, 1 H) 7.67-7.80 (m, 1 H) 7.95 (d, J = 2.45 Hz, 1 H) 7.99-8.14 (m, 1 H) 8.27 (d, J = 2.45 Hz, 1 H) 8.53 (d, J = 1.51 Hz, 1 H) 8.80 (s, 1 H) 10.40 (s, 1 H) 13.10 (s, 1 H) | methyl 5- (4,4,5,5- tetramethyl- 1,3,2- dioxaborolan-2- yl)-2-(2,2,2- trifluoroethoxy) nicotinate Intermediate 469 and Intermediate 115 |
| Example 1136 | 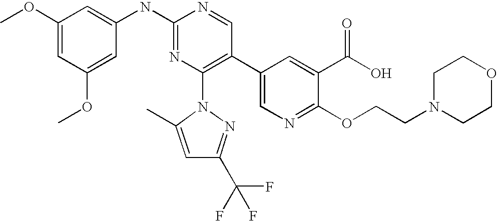 5-(2-(3,5-dimethoxyphenylamino)-4-(5- methyl-3-(trifluoromethyl)-1H-pyrazol- 1-yl)pyrimidin-5-yl)-2-(2- morpholinoethoxy)nicotinic acid | MS: ES+ 630.59 for C₂₉H₃₀F₃N₇O₆ 1H NMR (300 MHz, DMSO-d₆) δ ppm 2.35 (s, 3 H) 3.09-4.29 (m, 10 H) 3.74 (s, 6 H) 4.70 (br. s., 2 H) 6.14-6.34 (m, 1 H) 6.77 (s, 1 H) 7.06 (d, J = 2.07 Hz, 2 H) 7.74 (d, J = 2.45 Hz, 1 H) 8.22 (d, J = 2.45 Hz, 1 H) 8.93 (s, 1 H) 10.20 (s, 1 H) | methyl 2-(2- morpholino- ethoxy)-5-(4,4,5,5- tetramethyl- 1,3,2- dioxaborolan-2- yl)nicotinate Intermediate 468 and Intermediate 216 |
| Example 1137 | 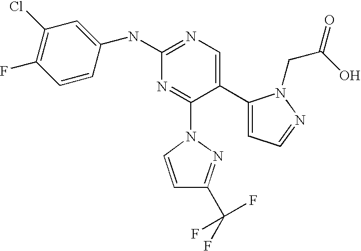 2-(5-(2-(3-chloro-4- fluorophenylamino)-4-(3- (trifluoromethyl)-1H-pyrazol-1- yl)pyrimidin-5-yl)-1H-pyrazol-1- yl)acetic acid | MS: ES+ 482.79 C₁₉H₁₂ClF₄N₇O₂ 1H NMR (300 MHz, DMSO-d₆) δ ppm 4.94 (s, 2 H) 7.06 (d, J = 2.45 Hz, 1 H) 7.24 (s, 1 H) 7.39 (s, 1 H) 7.68 (s, 2 H) 7.95-8.17 (m, 1 H) 8.39 (d, J = 1.70 Hz, 1 H) 8.93 (s, 1 H) 10.33 (s, 1 H) 13.07 (s, 1 H) | ethyl 2-(4- (4,4,5,5- tetramethyl- 1,3,2- dioxaborolan-2- yl)-1H-pyrazol- 1-yl)acetate and Intermediate 115 |
| Example 1138 | 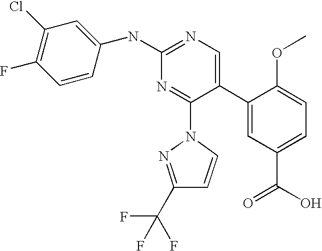 3-(2-(3-chloro-4-fluorophenylamino)-4- (3-(trifluoromethyl)-1H-pyrazol-1- yl)pyrimidin-5-yl)-4-methoxybenzoic acid | MS: ES+ 508.82 for C₂₂H₁₄ClF₄N₅O₃ H NMR (300 MHz, DMSO-d6) d ppm 3.44 (br. s., 3 H) 6.97 (br. s., 2 H) 7.41 (br. s., 1 H) 7.73 (br. s., 1 H) 7.87 (br. s., 2 H) 8.10 (br. s., 1 H) 8.42 (br. s., 1 H) 8.67 (br. s., 1 H) 10.34 (br. s., 1 H) 12.35- 13.05 (m, 1 H) | 2-methoxy-5- (methoxycarbonyl) phenylboronic acid and Intermediate 115 |
| Example 1139 | 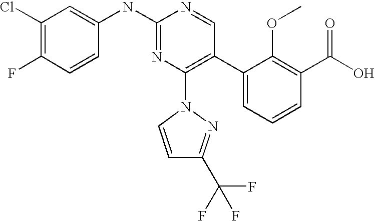 3-(2-(3-chloro-4-fluorophenylamino)-4- (3-(trifluoromethyl)-1H-pyrazol-1- yl)pyrimidin-5-yl)-2-methoxybenzoic acid | MS: ES+ 508.82 for C₂₂H₁₄ClF₄N₅O₃ 1H NMR (300 MHz, DMSO-d₆) δ ppm 3.37 (br. s., 3 H) 6.97 (br. s., 1 H) 7.21 (br. s., 1 H) 7.41 (br. s., 3 H) 7.71 (br. s., 2 H) 8.11 (br. s., 1 H) 8.46 (br. s., 1 H) 8.66 (br. s., 1 H) 10.35 (br. s., 1 H) 12.75-13.01 (m, 1 H) | 3-borono-2- methoxybenzoic acid and Intermediate 115 |
| Example 1140 | 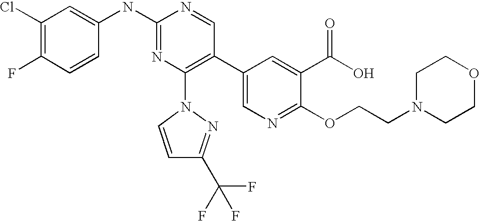 5-(2-(3-chloro-4-fluorophenylamino)-4- (3-(trifluoromethyl)-1H-pyrazol-1- yl)pyrimidin-5-yl)-2-(2- morpholinoethoxy)nicotinic acid | MS: ES+ 608.94 (M + 1) C₂₆H₂₂ClF₄N₇O₄ 1H NMR (300 MHz, DMSO-d₆) δ ppm 3.18-4.06 (m, 10 H) 4.66- 4.80 (m, 2 H) 7.04 (d, J = 2.64 Hz, 1 H) 7.42 (t, J = 9.14 Hz, 1 H) 7.63-7.78 (m, 1 H) 7.93 (d, 1 H) 8.08 (dd, J = 6.78, 2.64 Hz, 1 H) 8.29 (d, J = 2.45 Hz, 1 H) 8.52 (d, J = 1.70 Hz, 1 H) 8.79 (s, 1 H) 10.41 (s, 1 H) | methyl 2-(2- morpholinoethoxy)- 5-(4,4,5,5- tetramethyl- 1,3,2- dioxaborolan-2- yl)nicotinate Intermediate 468 and Intermediate 115 |
| Example 1141 | 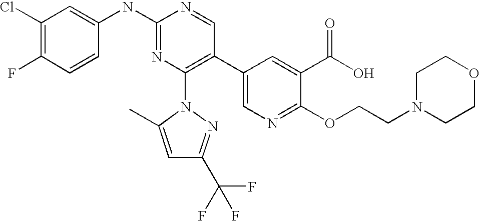 5-(2-(3-chloro-4-fluorophenylamino)-4- (5-methyl-3-(trifluoromethyl)-1H- pyrazol-1-yl)pyrimidin-5-yl)-2-(2- morpholinoethoxy)nicotinic acid | MS: ES+ 622.97 (m + 1) C₂₇H₂₄ClF₄N₇O₄ 1H NMR (300 MHz, DMSO-d₆) δ ppm 2.38 (s, 3 H) 3.20-4.09 (m, 8 H) 3.62 (d, J = 4.90 Hz, 2 H) 4.70 (br. s., 2 H) 6.78 (s, 1 H) 7.38-7.51 (m, 1 H) 7.61-7.70 (m, 1 H) 7.73 (d, J = 2.64 Hz, 1 H) 8.02-8.15 (m, 1 H) 8.23 (d, J = 2.45 Hz,1 H) 8.96 (s, 1 H) 10.44 (s, 1 H) | methyl 2-(2- morpholino- ethoxy)-5-(4,4,5,5- tetramethyl- 1,3,2- dioxaborolan-2- yl)nicotinate Intermediate 468 and Intermediate 113 |
| Example 1142 | 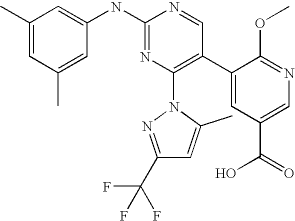 5-(2-(3,5-dimethylphenylamino)-4-(5- methyl-3-(trifluoromethyl)-1H-pyrazol- 1-yl)pyrimidin-5-yl)-6-methoxynicotinic acid | MS: ES+ 499.46 (M + 1) C₂₄H₂₁F₃N₆O₃ 1H NMR (300 MHz, DMSO-d₆) δ ppm 1.90 (s, 3 H) 2.26 (s, 6 H) 3.52 (s, 3 H) 6.69 (s, 1 H) 7.36 (s, 2 H) 8.00-8.11 (m, 1 H) 8.45-8.61 (m, 2 H) 8.65-8.82 (m, 1 H) 9.93-10.06 (m, 1 H) | methyl 5-bromo- 6- methoxynicotinate Intermediate 470 and Intermediate 218 |
| Example 1143 | 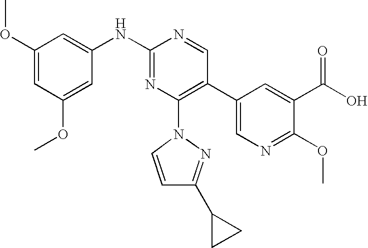 5-(4-(3-cyclopropyl-1H-pyrazol-1-yl)-2- (3,5-dimethoxyphenylamino)pyrimidin- 5-yl)-2-methoxynicotinic acid | MS: ES+ 489.50 (M + 1) for C₂₅H₂₄N₆O₅ 1H NMR (300 MHz, DMSO-d6) δ ppm 0.15 (d, J = 2.45 Hz, 2 H) 0.49 (dd, J = 8.10, 2.26 Hz, 2 H) 1.31- 1.54 (m, 1 H) 3.51 (s, 6 H) 3.72 (s, 3 H) 5.95 (br. s., 1 H) 6.15 (d, J = 2.45 Hz, 1 H) 6.84 (s, 2 H) 7.68 (d, J = 2.26 Hz, 1 H) 7.95 (d, J = 2.26 Hz, 1 H) 8.07 (d, J = 2.45 Hz, 1 H) 8.28 (s, 1 H) 9.65 (s, 1 H) 12.50- 12.85 (m, 1 H) | methyl 2- methoxy-5- (4,4,5,5- tetramethyl- 1,3,2- dioxaborolan-2- yl)nicotinate Intermediate 175 And methyl 5-(4-(3- cyclopropyl-1H- pyrazol-1-yl)-2- (3,5- dimethoxyphenyl- amino)pyrimidin- 5-yl)-2- methoxynicotinate Intermediate 471 |
| Example 1144 | 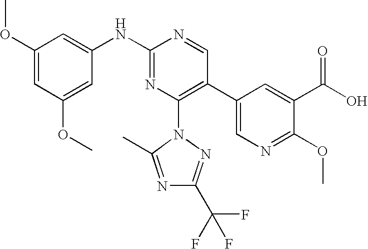 5-(2-(3,5-dimethoxyphenylamino)-4-(5- methyl-3-(trifluoromethyl)-1H-1,2,4- triazol-1-yl)pyrimidin-5-yl)-2- methoxynicotinic acid | MS: ES+ 531.44 (M + 1) for C₂₃H₂₀F₃N₇O₅ 1H NMR (300 MHz, DMSO-d6) δ ppm 2.56 (s, 3 H) 3.73 (s, 6 H) 3.93 (s, 3 H) 6.07-6.30 (m, 1 H) 7.03 (d, J = 2.26 Hz, 2 H) 7.66-7.89 (m, 1 H) 8.12-8.36 (m, 1 H) 8.95 (s, 1 H) 10.05- 10.41 (m, 1 H) 12.68-13.13 (m, 1 H) | methyl 2- methoxy-5- (4,4,5,5- tetramethyl- 1,3,2- dioxaborolan-2- yl)nicotinate Intermediate 175 and 5-bromo-N-(3,5- dimethoxyphenyl)- 4-(5-methyl- 3- (trifluoromethyl)- 1H-1,2,4- triazol-1- yl)pyrimidin-2- amine Intermediate 472 |
| Example 1145 | 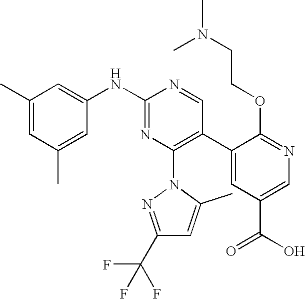 6-(2-(dimethylamino)ethoxy)-5-(2-(3,5- dimethylphenylamino)-4-(5-methyl-3- (trifluoromethyl)-1H-pyrazol-1- yl)pyrimidin-5-yl)nicotinic acid | MS: ES+ 556.55 (M + 1) for C₂₇H₂₈F₃N₇O₃ 1H NMR (300 MHz, DMSO-d6) δ ppm 2.27 (s, 6 H) 2.57 (s, 3 H) 2.74 (s, 6 H) 3.28-3.36 (m, 2) 4.29-4.47 (m, 2 H) 6.72 (d, J = 2.83 Hz, 2 H) 7.36 (s, 2 H) 8.01 (d, J = 2.26 Hz, 1 H) 8.67 (d, J = 2.26 Hz, 1 H) 8.79 (s, 1 H) 9.38-9.59 (m, 1 H) 10.03 (s, 1 H) 12.88- 13.43 (m, 1 H) | methyl 6-(2- (dimethylamino) ethoxy)-5- (4,4,5,5- tetramethyl- 1,3,2- dioxaborolan-2- yl)nicotinate Intermediate 476-B And Intermediate 218 |

The compounds in the below table were prepared using the procedure described for Example 1 and the specified starting materials.

| | | Mass spectrum and ¹H | |
|---|---|---|---|
| Compound | Structure | NMR | SM |
| Example 1146 | 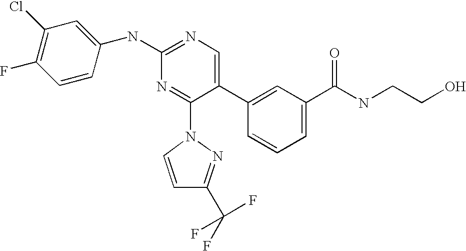 3-(2-(3-chloro-4- fluorophenylamino)-4-(3- (trifluoromethyl)-1H- pyrazol-1-yl)pyrimidin-5-yl)- N-(2- hydroxyethyl)benzamide | MS: 521.89 ES+ for C₂₃H₁₇ClF₄N₆O₂ 1H NMR (300 MHz, DMSO-d6) δ ppm 3.33 (d, J = 5.84 Hz, 2 H) 3.51 (d, J = 5.84 Hz, 2 H) 4.71 (t, J = 5.65 Hz, 1 H) 6.98 (d, J = 2.83 Hz, 1 H) 7.16 (d, J = 7.91 Hz, 1 H) 7.31-7.46 (m, 2 H) 7.57-7.74 (m, 1 H) 7.75-7.91 (m, 2 H) 8.13 (dd, J = 6.78, 2.64 Hz, 1 H) 8.34 (d, J = 1.70 Hz, 1 H) 8.43 (s, 1 H) 8.82 (s, 1 H) 10.41 (s, 1 H) | N(2- hydroxyethyl)-3- (4,4,5,5- tetramethyl1,3,2di- oxaborolan- 2yl)benzamide And Intermediate 115 |
| Example 1147 | 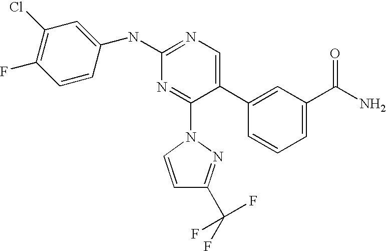 3-(2-(3-chloro-4- fluorophenylamino)-4-(3- (trifluoromethyl)-1H- pyrazol-1-yl)pyrimidin-5- yl)benzamide | MS: ES+ 477.81 for C₂₁H₁₃ClF₄N₆O 1H NMR (300 MHz, DMSO-d6) δ ppm 6.98 (d, J = 2.64 Hz, 1 H) 7.18-7.24 (m, 1 H) 7.41 (d, J = 2.83 Hz, 3 H) 7.67-7.76 (m, 1 H) 7.78 (s, 1 H) 7.81-7.88 (m, 2 H) 7.88-8.00 (m, 1 H) 8.06-8.19 (m, 1 H) 8.26-8.43 (m,1 H) 8.82 (s, 1 H) 10.41 (s, 1 H) | 3- carbamoylphenyl boronic acid And Intermediate 115 |
| Example 1148 | 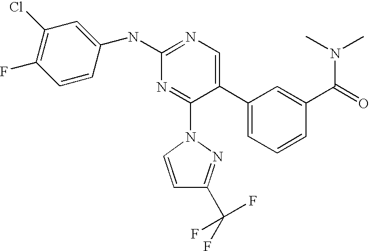 3-(2-(3-chloro-4- fluorophenylamino)-4-(3- (trifluoromethyl)-1H- pyrazol-1-yl)pyrimidin-5-yl)- N,N-dimethylbenzamide | MS: ES+ 505.87 for C₂₃H₁₇ClF₄N₆O 1H NMR (300 MHz, DMSO-d6) δ ppm 2.73- 3.01 (m, 6 H) 6.94-7.04 (m, 1 H) 7.10-7.19 (m, 1 H) 7.42 (d, J = 8.85 Hz, 4 H) 7.67-7.78 (m, 1 H) 8.06- 8.13 (m, 1 H) 8.34-8.46 (m, 1 H) 8.75 (s, 1 H) 10.15-10.48 (m, 1 H) | 3- (dimethylcarbamoyl) phenylboronic acid And Intermediate 115 |
| Example 1149 | 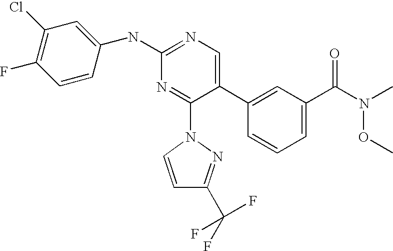 3-(2-(3-chloro-4- fluorophenylamino)-4-(3- (trifluoromethyl)-1H- pyrazol-1-yl)pyrimidin-5-yl)- N-methoxy-N- methylbenzamide | MS: ES+ 521.87 for C₂₃H₁₇ClF₄N₆O₂ 1H NMR (300 MHz, DMSO-d6) δ ppm 3.22 (s, 3 H) 3.50 (s, 3 H) 6.93- 7.08 (m, 1 H) 7.26-7.50 (m, 5 H) 7.51-7.61 (m, 1 H) 7.66-7.87 (m, 2 H) 8.06-8.18 (m, 1 H) 8.32- 8.41 (m, 1 H) 8.77 (s, 1 H) 10.40 (s, 1 H) | 3- (methoxy(methyl) carbamoyl)phenyl boronic acid And Intermediate 115 |
| Example 1150 | 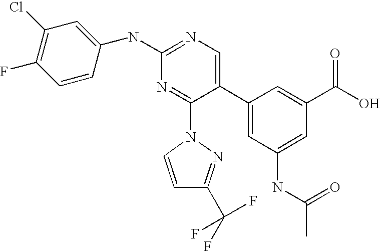 3-acetamido-5-(2-(3-chloro- 4-fluorophenylamino)-4-(3- (trifluoromethyl)-1H- pyrazol-1-yl)pyrimidin-5- yl)benzoic acid | MS: ES+ 535.85 for C₂₃H₁₅ClF₄N₆O₃ 1H NMR (300 MHz, DMSO-d6) δ ppm 2.05 (s, 3 H) 6.99 (d, J = 2.64 Hz, 1 H) 7.33 (s, 1 H) 7.42 (s, 1 H) 7.69 (s, 2 H) 8.16 (s, 2 H) 8.28-8.49 (m, 1 H) 8.75 (s, 1 H) 10.00-10.23 (m, 1 H) 10.41 (s, 1 H) 12.85-13.06 (m, 1 H) | 3-acetamido-5- boronobenzoic acid And Intermediate 115 |
| Example 1151 | 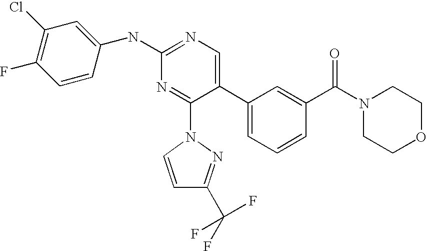 (3-(2-(3-chloro-4- fluorophenylamino)-4-(3- (trifluoromethyl)-1H- pyrazol-1-yl)pyrimidin-5- yl)phenyl)(morpholino) methanone | MS: ES+ 546.90 for C₂₅H₁₉ClF₄N₆O₂ 1H NMR (300 MHz, DMSO-d6) δ ppm 3.35- 3.69 (m, 8 H) 6.97-7.04 (m, 1 H) 7.14-7.19 (m, 1 H) 7.30-7.52 (m, 5 H) 7.61- 7.80 (m, 1 H) 8.04-8.15 (m, 1 H) 8.36-8.45 (m, 1 H) 10.16-10.66 (m, 1 H) | 3-(morpholine-4- carbonyl)phenyl- boronic acid And Intermediate 115 |
| Example 1152 | 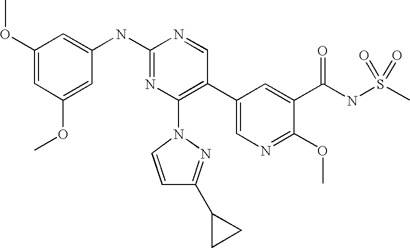 5-(4-(3-cyclopropyl-1H- pyrazol-1-yl)-2-(3,5- dimethoxyphenylamino) pyrimidin-5-yl)-2-methoxy-N- (methylsulfonyl)nicotinamide | MS: ES+ 566.60 for (M + 1) C₂₆H₂₇N₇O₆S 1H NMR (300 MHz, DMSO-d6) δ ppm 0.36- 0.52 (m, 2 H) 0.69-0.83 (m, 2 H) 1.63-1.84 (m, 1 H) 3.35 (s, 3 H) 3.75 (s, 6 H) 4.00 (s, 3 H) 6.13-6.26 (m, 1 H) 6.31-6.46 (m, 1 H) 7.03-7.15 (m, 2 H) 7.85-7.97 (m, 1 H) 8.15- 8.24 (m, 1 H) 8.26-8.36 (m, 1 H) 8.49-8.58 (m, 1 H) 9.95 (s, 1 H) 11.67 (s, 1 H) | 5-bromo-4-(3- cyclopropyl-1H- pyrazol-1-yl)-N- (3,5- dimethoxyphenyl) pyrimidin-2- amine Intermediate 368 and Intermediate 471 |
| Example 1153 | 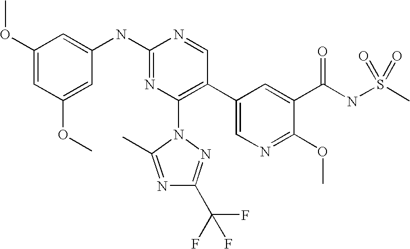 5-(2-(3,5- dimethoxyphenylamino)-4- (5-methyl-3- (trifluoromethyl)-1H-1,2,4- triazol-1-yl)pyrimidin-5-yl)- 2-methoxy-N- (methylsulfonyl)nicotinamide | MS: ES+ 609.55 for (M + 1) C₂₄H₂₃F₃N₈O₆S 1H NMR (300 MHz, DMSO-d₆) δ ppm 2.56 (s, 3 H) 3.33 (s, 3 H) 3.73 (s, 6 H) 3.95 (s, 3 H) 6.12-6.33 (m, 1 H) 7.03 (d, J = 2.07 Hz, 2 H) 7.72-7.98 (m, 1 H) 8.01-8.18 (m, 1 H) 8.94 (s, 1 H) 10.08-10.39 (m, 1 H) 11.67-11.81 (m, 1 H) | 2-methoxy-N- (methylsulfonyl)- 5-(4,4,5,5- tetramethyl-1,3,2- dioxaborolan-2- yl)nicotinamide Intermediate 368 and 5-bromo-N-(3,5- dimethoxyphenyl)- 4-(5-methyl-3- (trifluoromethyl)- 1H-1,2,4-triazol- 1-yl)pyrimidin-2- amine Intermediate 472 |
| Example 1154 | 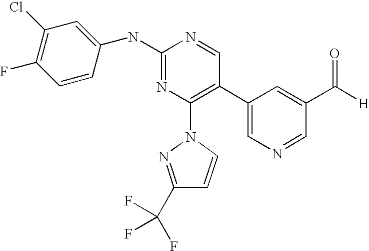 5-(2-(3-chloro-4- fluorophenylamino)-4-(3- (trifluoromethyl)-1H- pyrazol-1-yl)pyrimidin-5- yl)nicotinaldehyde | MS: ES+ 463.79 for (M + 1) C₂₀H₁₁ClF₄N₆O | 5-bromo-N-(3- chloro-4- fluorophenyl)-4- (3- (trifluoromethyl)- 1H-pyrazol-1- yl)pyrimidin-2- amine Intermediate 115 And 5-(4,4,5,5- tetramethyl-1,3,2- dioxaborolan-2- yl)nicotinaldehyde |

### Example 1155

### (E)-methyl 3-(5-(2-(3-chloro-4-fluorophenylamino)-4-(3-(trifluoromethyl)-1H-pyrazol-1-yl)pyrimidin-5yl)pyridin-3-yl)acrylate

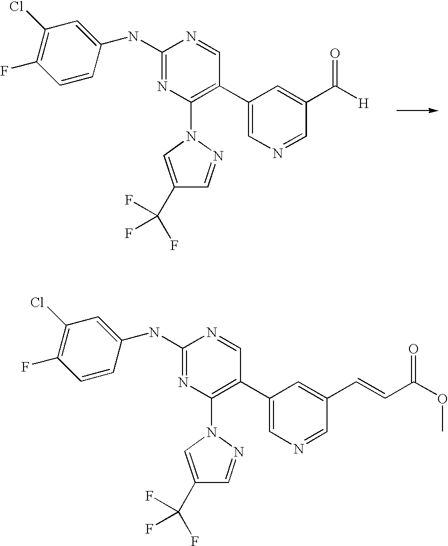

Methyl 2-(diethoxyphosphoryl)acetate (0.102 mL, 0.51 mmol) was dissolved in THF (1 mL). NaH, 60% dispersion in oil, (30.7 mg, 0.77 mmol) was added and the suspension was stirred for 5 minutes to give a solution. The solution was then added to a mixture of 5-(2-(3-chloro-4-fluorophenylamino)-4-(3-(trifluoromethyl)-1H-pyrazol-1-yl)pyrimidin-5-yl)nicotinaldehyde Example 1154 (227 mg, 0.51 mmol) in THF (3 ml). The reaction mixture was then heated at 50° C. for 1 hour. Water was added and the mixture was extracted with ethyl acetate. The organic layer was washed with brine, then dried with MgSO4, filtered and concentrated. The residue was triturated with diethyl ether to give a solid mass, which was collected and further rinsed with diethyl ether to give the title compound as an off white solid. (227 mg).

MS (Electrospray): 519 (MH⁺) for C₂₃H₁₅ClF₄N₆O₂

### Example 1156

### (E)-3-(5-(2-(3-chloro-4-fluorophenylamino)-4-(3-(trifluoromethyl)-1H-pyrazol-1-yl)pyrimidin-5-yl)pyridin-3-yl)acrylic acid

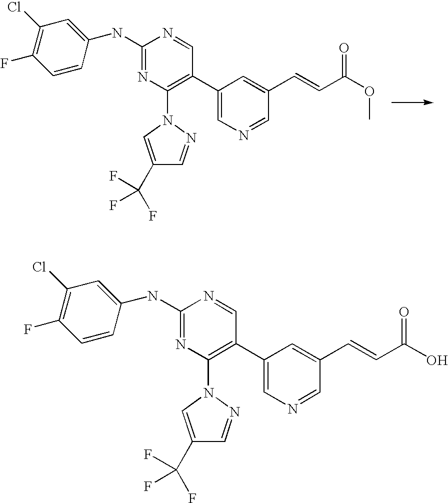

(E)-methyl 3-(5-(2-(3-chloro-4-fluorophenylamino)-4-(3-(trifluoromethyl)-1H-pyrazol-1-yl)pyrimidin-5-yl)pyridin-3-yl)acrylate Example 1155 (100 mg, 0.19 mmol) and 2M KOH (0.289 mL, 0.58 mmol) were combined in Dioxane (2 mL). The reaction mixture was allowed to stir at RT overnight, then neutralized with 1M HCl to pH 6. Water and 10% methanol in ethyl acetate were added and the layers were separated. The organic layer was dried with MgSO4 then concentrated down to a residue which was purified by reverse phase chromatography (C18: 15-95% acetonitrile in water with 0.1% TFA) to give the title compound (10 mg).

MS (Electrospray): 505.28 (MH⁺) for C₂₂H₁₃ClF₄N₆O₂

1H NMR (300 MHz, DMSO-d6) δ ppm 6.61 (d, J=16.20 Hz, 1H) 7.06 (d, J=2.64 Hz, 1H) 7.43 (t, J=9.04 Hz,1H) 7.50-7.62 (m, 1H) 7.74 (ddd, J=7.35, 4.52, 4.14 Hz, 1H) 8.02 (s, 1H) 8.09 (dd, J=6.69, 2.54 Hz, 1H) 8.32 (d, J=1.88 Hz, 1H) 8.53 (s, 1H) 8.77 (d, J=1.70 Hz, 1H) 8.85 (s, 1H) 10.48 (s, 1H) 12.65 (br. s., 1H)

The compounds in the below table were prepared using the general method described above for Example 1 using the specified starting materials.

| Compound | Structure | Mass spectrum and ¹H NMR | SM |
|---|---|---|---|
| Example 1157 | 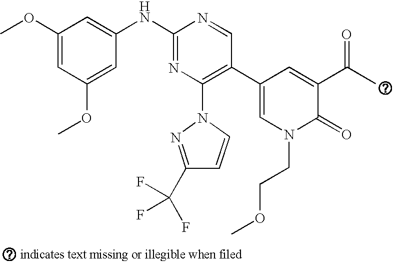 methyl 5-(2-(3,5-dimethoxyphenylamino)-4-(3- (trifluoromethyl)-1H-pyrazol-1-yl)pyrimidin-5- yl)-1-(2-methoxyethyl)-2-oxo-1,2-dihydropyridine- 3-carboxylate | MS(ES): 575 (M + 1) for C₂₆H₂₅F₃N₆O₆. 1H NMR (300 MHz, DMSO- d6) δ ppm 3.23 (s, 3 H) 3.59 (t, J = 5.37 Hz, 2 H) 3.69 (s, 3 H) 3.74 (s, 6 H) 4.10 (t, J = 5.46 Hz, 2 H) 6.21 (t, J = 2.17 Hz, 1 H) 6.99-7.14 (m, 3 H) 7.70 (d, J = 2.64 Hz, 1 H) 8.01 (d, J = 2.83 Hz, 1 H) 8.43-8.60 (m, 1 H) 8.68 (s, 1 H) 10.12 (s, 1 H) | methyl 1-(2- methoxyethyl)-2- oxo-5-(4,4,5,5- tetramethyl-1,3,2- dioxaborolan-2-yl)- 1,2-dihydropyridine- 3-carboxylate Intermediate 476 and 5-bromo-N-(3,5- dimethoxyphenyl)-4-(3- (trifluoromethyl)- 1H-pyrazol-1- yl)pyrimidin-2- amine Intermediate 215 |
| Example 1158 | 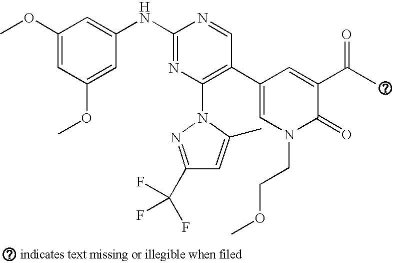 methyl 5-(2-(3,5-dimethoxyphenylamino)-4-(5- methyl-3-(trifluoromethyl)-1H-pyrazol-1- yl)pyrimidin-5-yl)-1(2-methoxyethyl)-2-oxo- 1,2-dihydropyridine-3-carboxylate | MS(ES): 589 (M + 1) for C₂₇H₂₇F₃N₆O₆. 1H NMR (300 MHz, DMSO- d6) δ ppm 2.32 (s, 3 H) 3.22 (s, 3 H) 3.56 (t, J = 5.27 Hz, 2 H) 3.66 (s, 3 H) 3.72 (s, 6 H) 3.95-4.19 (m, 2 H) 6.21 (t, J = 2.26 Hz, 1 H) 6.77 (s, 1 H) 7.03 (d, J = 2.26 Hz, 2 H) 7.35 (d, J = 2.83 Hz, 1 H) 8.01 (d, J = 2.83 Hz, 1 H) 8.82 (s, 1 H) 10.16 (s, 1 H)) | methyl 1-(2- methoxyethyl)-2- oxo-5-(4,4,5,5- tetramethyl-1,3,2- dioxaborolan-2-yl)- 1,2-dihyropyridine- 3-carboxylate Intermediate 476 And 5-bromo-N- (3,5- dimethoxyphenyl)- 4-(5-methyl-3- (trifluoromethyl)- 1H-pyrazol-1- yl)pyrimidin-2- amine Intermediate 216 |

The compounds in the below table were prepared using the general method described above for

Example 214 using 1N sodium hydroxide (2 equivalents), dioxane : THF (1:1) as solvent and the specified starting material.

| Compound | Structure | Mass spectrum and ¹H NMR | SM |
|---|---|---|---|
| Example 1159 | 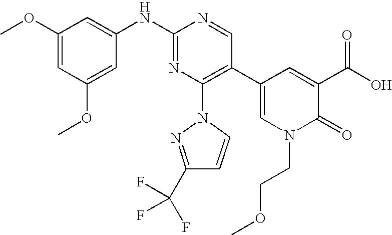 5-(2-3,5-dimethoxyphenylamino)-4-(3-(trifluoromethyl)- 1H-pyrazol-1-yl)pyrimidin-5-yl)-1-(2-methoxyethyl)- 2-oxo-1,2-dihydropyridine-3-carboxylic acid | MS(ES): 561 (M + 1) for C₂₅H₂₃F₃N₆O₆. 1H NMR (300 MHz, DMSO- d6) δ ppm 3.23 (s, 3 H) 3.67 (t, J = 5.27 Hz, 2 H) 3.75 (s, 6 H) 4.29 (t, J = 5.37 Hz, 2 H) 6.22 (t, J = 2.17 Hz, 1 H) 6.97-7.16 (m, 3 H) 8.11 (d, J = 2.64 Hz, 1 H) 8.27 (d, J = 2.64 Hz, 1 H) 8.57 (dd, 1 H) 8.69 (s, 1 H) 10.17 (s, 1 H) | methyl 5-(2-(3,5- dimethoxyphenyl- amino)-4-(3- (trifluoromethyl)- 1H-pyrazol-1- yl)pyrimidin- 5-yl)-1-)2- methoxyethyl)-2-oxo- 1,2-dihydropyridine-3- carboxylate Example 1157 |
| Example 1160 | 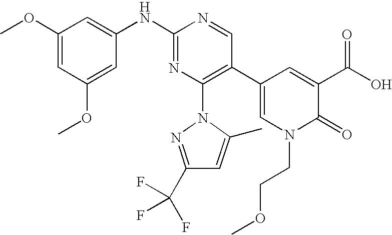 5-(2-(3,5-dimethoxyphenylamino)-4-(5-methyl-3- (trifluoromethyl)-1H-pyrazol-1-yl)pyrimidin-5- yl)-1-(2-methoxyethyl)-2-oxo-1,2- dihydropyridine-3-carboxylic acid | MS(ES): 575 (M + 1) for C₂₆H₂₅F₃N₆O₆. 1H NMR (300 MHz, DMSO- d6) δ ppm 2.45 (s, 3 H) 3.23 (s, 3 H) 3.65 (t, J = 5.27 Hz, 2 H) 3.73 (s, 6 H) 4.27 (t, J = 5.27 Hz, 2 H) 6.22 (t, J = 2.17 Hz, 1H) 6.79 (s, 1 H) 7.03 (d, J = 2.07 Hz, 2 H) 7.75 (d, J = 2.64 Hz, 1 H) 8.25 (d, J = 2.83 Hz, 1 H) 8.82 (s, 1 H) 10.17 (s, 1 H) 14.27 (s, 1 H) | methyl 5-(2-(3,5- dimethoxyphenyl- amino)-4-(5-methyl-3- (trifluoromethyl)-1H- pyrazol-1-yl)pyrimidin- 5-yl)-1-(2- methoxyethyl)-2-oxo- 1,2-dihydropyridine-3- carboxylate Example 1158 |

The compounds in the below table were prepared using the general method described above for

Example 800 using 2-chloro-1-methylpyridinium iodide, 4-(Dimethylamino)pyridine and triethylamine, with CH₂Cl₂ as solvent and the carboxylic acid and sulfonamide starting materials listed.

| Compound | Structure | Mass spectrum and ¹H NMR | SM |
|---|---|---|---|
| Example 1161 | 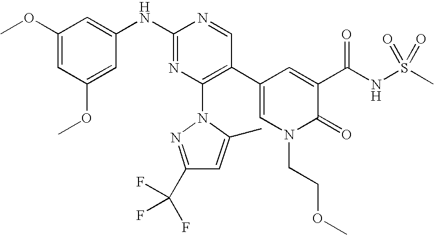 5-(2-(3,5-dimethoxyphenylamino)-4-(5-methyl-3- (trifluoromethyl)-1H-pyrazol-1-yl)pyrimidin-5-yl)-1- (2-methoxyethyl)-N-(methylsulfonyl)-2-oxo-1,2- dihydropyridine-3-carboxamide | MS(ES): 652 (M + 1) for C₂₇H₂₈F₃N₇O₇S. 1H NMR (300 MHz, DMSO- d6) δ 2.43 (s, 3 H) 3.23 (s, 3 H) 3.35 (s, 3 H) 3.62 (t, J = 5.27 Hz, 2 H) 3.73 (s, 6 H) 4.11-4.35 (m, 2H) 6.22 (t, J = 2.17 Hz, 1 H) 6.79 (s, 1 H) 7.03 (d, J = 2.26 Hz, 2 H) 7.83 (d, J = 2.64 Hz, 1 H) 8.23 (d, J = 2.45 Hz, 1 H) 8.83 (s, 1 H) 10.18 (s, 1 H) 12.61 (s, 1 H) | 5-(2-(3,5- dimethoxyphenyl- amino)-4-(5-methyl-3- (trifluoromethyl)-1H- pyrazol-1- yl)pyrimidin-5-yl)-1- (2-methoxyethyl)-2- oxo-1,2- dihydropyridine-3- carboxylic acid Example 1160 And Methanesulfonamide |
| Example 1162 | 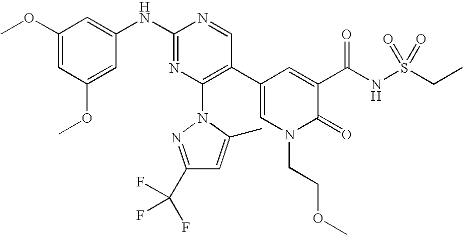 5-(2-(3,5-dimethoxyphenylamino)-4-(5-methyl-3- (trifluoromethyl)-1H-pyrazol-1-yl)pyrimidin-5-yl)- N-(ethylsulfonyl)-1-(2-methoxyethyl)-2-oxo-1,2- dihydropyridiine-3-carboxamide | MS(ES): 666 (M + 1) for C₂₈H₃₀F₃N₇O₇S. 1H NMR (300 MHz, DMSO- d6) δ 1.22 (t, J = 7.35 Hz, 3 H) 2.45 (s, 3 H) 3.23 (s, 3 H) 3.41- 3.56 (m, 2 H) 3.63 (t, J = 5.09 Hz, 2 H) 3.73 (s, 6 H) 4.24 (t, J = 5.18 Hz, 2 H) 6.22 (t, J = 2.17 Hz, 1 H) 6.79 (s, 1 H) 7.03 (d, J = 2.07 Hz, 2 H) 7.79 (d, J = 2.64 Hz, 1 H) 8.25 (d, J = 1.88 Hz, 1 H) 8.83 (s, 1 H) 10.17 (s, 1 H) 12.56 (s, 1 H) | 5-(2-(3,5- dimethoxyphenyl- amino)-4-(5-methyl-3- (trifluoromethyl)-1H- pyrazol-1- yl)pyrimidin-5-yl)-1- (2-methoxyethyl)-2- oxo-1,2- dihydropyridine-3- carboxylic acid Example 1160 And Ethanesulfonamide |
| Example 1163 | 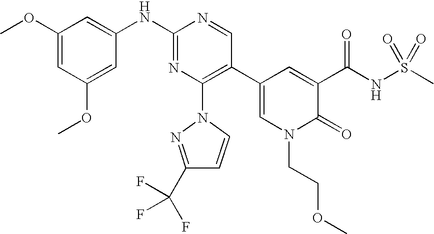 5-(2-(3,5-dimethoxyphenylamino)-4-(3-(trifluoromethyl)- 1H-pyrazol-1-yl)pyrimidin-5-yl)-1-(2-methoxyethyl)-N- (methylsulfonyl)-2-oxo-1,2-dihydropyridine-3-carboxamide | MS(ES): 638 (M + 1) for C₂₆H₂₆F₃N₇O₇S. 1H NMR (300 MHz, DMSO- d6) δ ppm 3.23 (s, 3 H) 3.36 (s, 3 H) 3.65 (t, J = 5.27 Hz, 2 H) 3.75 (s, 6 H) 4.25 (t, J = 4.14 Hz, 2 H) 6.22 (t, J = 2.26 Hz, 1 H) 6.95-7.14 (m, 3 H) 8.16 (br. s., 1 H) 8.27 (br. s., 1 H) 8.56 (s, 1 H) 8.70 (s, 1 H) 10.17 (s, 1 H) 12.73 (s, 1 H)) | 5-(2-(3,5- dimethoxyphenyl- amino)-4-(3- (trifluoromethyl)-1H- pyrazol-1- yl)pyrimidin-5-yl)-1- (2-methoxyethyl)-2- oxo-1,2- dihydropyridine-3- carboxylic acid Example 1159 And Methanesulfonamide |
| Example 1164 | 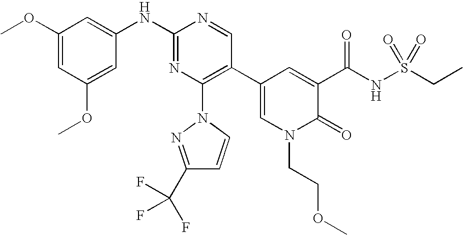 5-(2-(3,5-dimethoxyphenylamino)-4-(5-methyl-3-(trifluoromethyl)- 1H-pyrazol-1-yl)pyrimidin-5-yl)-1-(2-methoxyethyl)-N- (methylsulfonyl)-2-oxo-1,2-dihydropyridine-3-carboxamide | MS(ES): 652(M + 1) for C₂₇H₂₈F₃N₇O₇S. 1H NMR (300 MHz, DMSO- d6) δ 1.22 (t, J = 7.35 Hz, 3 H) 3.23 (s, 3 H) 3.49 (q, J = 7.66 Hz, 2 H) 3.66 (t, J = 5.18 Hz, 2 H) 3.75 (s, 6 H) 4.26 (t, J = 5.09 Hz, 2 H) 6.22 (t, J = 2.17 Hz, 1 H) 6.69 (s, 1 H) 7.08 (d, J = 2.07 Hz, 2 H) 8.13 (d, J = 2.26 Hz, 1 H) 8.28 (s, 1 H) 8.57 (s, 1 H) 8.69 (s, 1 H) 10.16 (s, 1 H) 12.68 (s, 1 H) | 5-(2-(3,5- dimethoxyphenyl- amino)-4-(3- (trifluoromethyl)-1H- pyrazol-1- yl)pyrimidin-5-yl)-1- (2-methoxyethyl)-2- oxo-1,2- dihydropyridine-3- carboxylic acid Example 1159 And Ethanesulfonamide |

The compound in the below table was prepared using the general method described above for Example 858 using the specified starting materials.

| Compound | Structure | Mass spectrum and ¹H NMR | SM |
|---|---|---|---|
| Example 1165 | 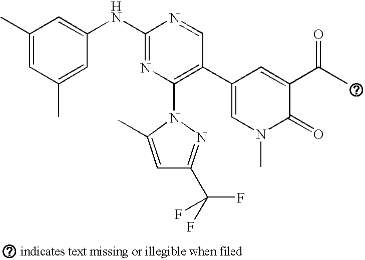 methyl 5-(2-(3,5-dimethylphenylamino)-4- (5-methyl-3-(trifluoromethyl)-1H-pyrazol- 1-yl)pyrimidin-5-yl)-1-methyl-2-oxo- 1,2-dihydropyridine-3-carboxylate | MS(ES): 513 (M + 1) for C₂₅H₂₃F₃N₆O₃. 1H NMR (300 MHz, DMSO- d6) δ ppm 2.25 (s, 6 H) 2.40 (s, 3 H) 3.47 (s, 3 H) 3.65 (s, 3 H) 6.68 (s, 1 H) 6.78 (s, 1 H) 7.28 (d, J = 2.83 Hz, 1 H) 7.34 (s, 2 H) 8.15 (d, J = 2.64 Hz, 1 H) 8.83 (s, 1 H) 10.03 (s, 1 H) | [5-(methoxycarbonyl)- 1-methyl-6-oxo-1,6- dihydropyridin-3- yl]boronic acid Intermediate 323 And 5-bromo-N-(3,5- dimethylphenyl)-4-(5- methyl-3- (trifluoromethyl)-1H- pyrazol-1- yl)pyrimidin-2-amine Intermediate 218 |

The compound in the below table was prepared using the general method described above for Example 859 using the specified starting material.

| Compound | Structure | Mass spectrum and ¹H NMR | SM |
|---|---|---|---|
| Example 1166 | 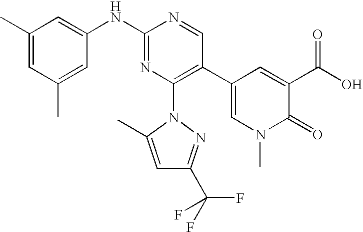 5-(2-(3,5-dimethylphenylamino)-4-(5-methyl-3- (trifluoromethyl)-1H-pyrazol-1-yl)pyrimidin- 5-yl)-1-methyl-2-oxo-1,2-dihydropyridine- 3-carboxylic acid | MS(ES): 499 (M + 1) for C₂₄H₂₁F₃N₆O₃. 1H NMR (300 MHz, DMSO- d6) δ ppm 2.26 (s, 6 H) 2.51 (br. s., 3 H) 3.67 (s, 3 H) 6.70 (s, 1 H) 6.79 (s, 1 H) 7.34 (s, 2 H) 7.65 (d, J = 2.64 Hz, 1 H) 8.40 (d, J = 2.64 Hz, 1 H) 8.83 (s, 1 H) 10.06 (s, 1 H) 14.39 (s, 1 H)) | methyl 5-(2-(3,5- dimethylphenyl- amino)-4-(5-methyl-3- (trifluoromethyl)-1H- pyrazol-1- yl)pyrimidin-5-yl)-1- methyl-2-oxo-1,2- dihydropyridine-3- carboxylate Example 1165 |

The following examples were prepared using the general HATU coupling method described for Example 360 using the starting materials (SM) indicated.

| Ex | Compound | Mass spectrum ¹NMR | SM |
|---|---|---|---|
| Example 1167 | 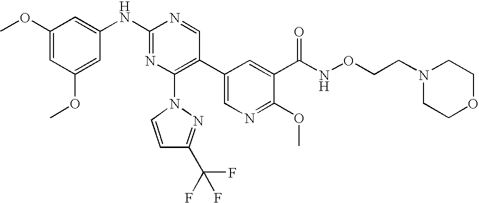 5-(2-(3,5-dimethoxyphenylamino)-4-(3-(trifluoromethyl)- 1H-pyrazol-1-yl)pyrimidin-5-yl)-2-methoxy-N- (2-morpholinoethoxy)nicotinamide | MS(ES): 645 (M + 1) for C₂₉H₃₁F₃N₈O₆ 1H NMR (300 MHz, DMSO- D6) δ ppm 3.21 (br s, 2 H) 3.40- 3.53 (m, 2 H) 3.60 (s, 2 H) 3.75 (s, 8 H) 3.93-4.07 (m, 5 H) 4.24 (t, 2 H) 6.22 (t, J = 2.17 Hz, 1 H) 7.04 (d, J = 2.64 Hz, 1 H) 7.10 (d, J = 2.07 Hz, 2 H) 7.91 (d, J = 2.45 Hz, 1 H) 8.21 (d, J = 2.45 Hz, 1 H) 8.48 (d, J = 1.70 Hz, 1 H) 8.66-8.89 (m, 1 H) 10.17 (s, 1 H) 11.67 (s, 1 H). | O-(2- morpholinoethyl)- hydroxylamine and 5-(2-(3,5- dimethoxyphenyl- amino)-4-(3- (trifluoromethyl)-1H- pyrazol-1- yl)pyrimidin-5-yl)-2- methoxynicotinic acid Example 675 |
| Example 1168 | 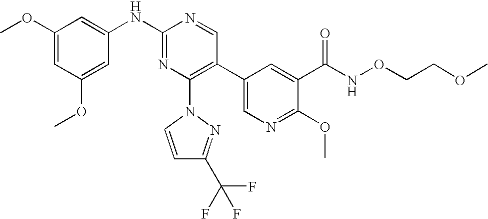 5-(2-(3,5-dimethoxyphenylamino)-4-(3-(trifluoromethyl)- 1H-pyrazol-1-yl)pyrimidin-5-yl)-2-methoxy-N- (2-methoxyethoxy)nicotinamide | MS (ES): 590 (M + 1) for C₂₆H₂₆F₃N₇O₆ 1H NMR (300 MHz, DMSO- D6) δ ppm 3.30 (s, 3 H) 3.51- 3.64 (m, 2 H) 3.75 (s, 6 H) 3.90- 4.09 (m, 5 H) 6.21 (t, J = 2.26 Hz, 1 H) 7.02 (d, J = 2.64 Hz, 1 H) 7.11 (d, J = 2.26 Hz, 2 H) 7.82 (d, J = 2.45 Hz, 1 H) 8.16 (d, J = 2.45 Hz, 1 H) 8.44 (d, J = 1.51 Hz, 1 H) 8.75 (s, 1 H) 10.16 (s, 1 H) 11.26 (s, 1 H). | O-(2-methoxyethyl)- hydroxylamine and 5-(2-(3,5- dimethoxyphenyl- amino)-4-(3- (trifluoromethyl)-1H- pyrazol-1- yl)pyrimidin-5-yl)-2- methoxynicotinic acid Example 675 |

### Example 1169

### 5-(2-methoxy-5-{2-[(3-methoxy-5-methylphenyl)amino]-4-[3-(trifluoromethyl)-1H-pyrazol-1-yl]pyrimidin-5-yl}pyridin-3-yl)-1,3,4-oxadiazol-2(3H)-one

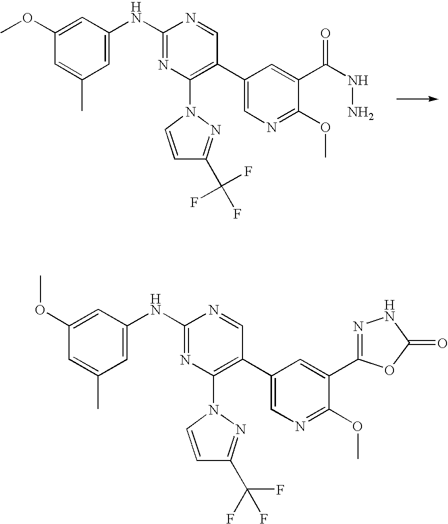

To a mixture of 2-methoxy-5-{2-[(3-methoxy-5-methylphenyl)amino]-4-[3-(trifluoromethyl)-1H-pyrazol-1-yl]pyrimidin-5-yl}pyridine-3-carbohydrazide (Intermediate 477, 0.58 mmol, 300 mg) and N,N-diisopropylethylamine (0.88 mmol, 0.16 mL) in DMF, was added 1,1′-carbonyldiimidazole (0.88 mmol, 142 mg) and the reaction mixture was stirred at RT for 1 h and further at 50° C. for another hour. The reaction mass was diluted with ethyl acetate (20 mL). The organic layer was separated, washed with water and brine, dried over Na₂SO₄, filtered and concentrated. The crude material was purified by silica gel column chromatography using ethyl acetate/hexanes (40:60) to obtain 90 mg of the title compound.

| Compound | Structure | Mass spectrum and ¹H NMR | SM |
|---|---|---|---|
| Example 1169 | 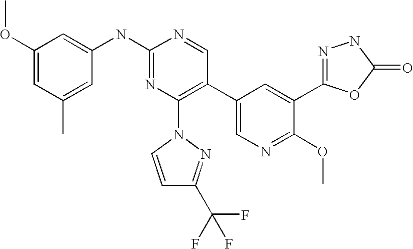 5-(2-methoxy-5-{2-[(3-methoxy-5-methylphenyl)amino]- 4-[3-(trifluoromethyl)-1H-pyrazol-1-yl]pyrimidin-5- yl}pyridin-3-yl)-1,3,4-oxadiazol-2(3H)-one | MS (ES): 541 (M + 1) for C₂₄H₁₉F₃N₈O₄. 400 MHz, DMSO-d₆: δ 2.28 (s, 3H), 3.74 (s, 3H), 3.99 (s, 3H), 6.46 (s, 1H), 7.03 (d, J = 2.60 Hz, 1H), 7.15 (s, 1H), 7.35 (s, 1H), 7.83 (d, J = 2.40 Hz, 1H), 8.25 (d, J = 2.40 Hz, 1H), 8.49 (d, J = 1.68 Hz, 1H), 8.77 (s, 1H), 10.14 (s, 1H), 12.64 (s, 1H). | Intermediate 477 2-methoxy-5-{2-[(3- methoxy-5- methylphenyl)amino]-4-[3- (trifluoromethyl)-1H- pyrazol-1-yl]pyrimidin-5- yl}pyridine-3- carbohydrazide |

### Example 1170

### 5-(2-methoxy-5-{2-[(3-methoxy-5-methylphenyl)amino]-4-[5-methyl-3-(trifluoromethyl)-1H-pyrazol-1-yl]pyrimidin-5-yl}pyridin-3-yl)-1,3,4-oxadiazol-2(3H)-one

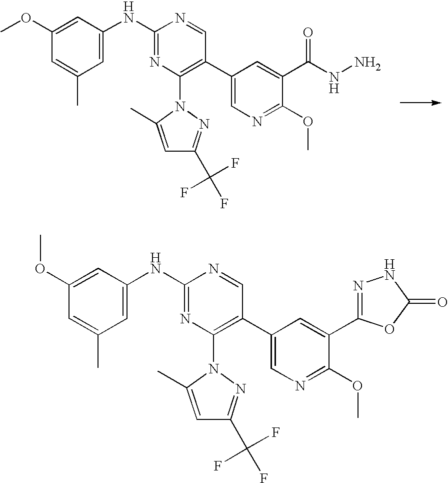

To a solution of the 2-methoxy-5-{2-[(3-methoxy-5-methylphenyl)amino]-4-[5-methyl-3-(trifluoromethyl)-1H-pyrazol-1-yl]pyrimidin-5-yl}pyridine-3-carbohydrazide (Intermediate 478, 0.13 mmol, 70 mg) in dry DMF(1 mL) was added N,N-diisopropylethylamine (0.20 mmol, 26 mg, 0.34 mL) and 1,1′-carbonyldiimidazole (0.20 mmol, 32 mg). The mixture was stirred for 1 h at RT and further heated at 50° C. for 1 h. The reaction mixture was diluted with DCM (15 mL) and further washed with water (25 mL) and brine (25 mL). The organic layer was dried over Na₂SO₄ and concentrated. The crude mass was purified by flash chromatography (product eluted with 40% EtOAc/hexanes) to afford the title compound as 35 mg of white solid.

| Compound | Structure | Mass spectrum and ¹H NMR | SM |
|---|---|---|---|
| Example 1170 | 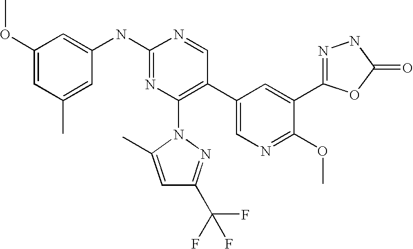 5-(2-methoxy-5-{2-[(3-methoxy-5-methylphenyl)amino]- 4-[5-methyl-3-(trifluoromethyl)-1H-pyrazol-1-yl]pyrimidin- 5-yl}pyridine-3-yl)-1,3,4-oxadiazol-2(3H)-one | MS(ES): 555 (M + 1) for C₂₅H₂₁F₃N₈O₄. 400 MHz, DMSO-d₆: δ 2.26 (s, 3H), 2.36 (s, 3H), 3.71 (s, 3H), 3.96 (s, 3H), 6.46 (s, 1H), 6.75 (s, 1H), 7.13 (s, 1H), 7.28 (s, 1H), 7.61 (d, J = 1.64 Hz, 1H), 8.13 (d, J = 1.60 Hz, 1H), 8.92 (s, 1H), 10.16 (s, 1H), 12.65 (s, 1H). | Intermediate 478 2-methoxy-5-{2-[(3- methoxy-5- methylphenyl)amino]-4-[5- methyl-3-(trifluoromethyl)- 1H-pyrazol-1-yl]pyrimidin- 5-yl}pyridine-3- carbohydrazide |

### Example 1171

### ethyl 1-ethyl-5-{2-[(3-methoxy-5-methylphenyl)amino]-4-[3-(trifluoromethyl)-1H-pyrazol-1-yl]pyrimidin-5-yl}-2-oxo-1,2-dihydropyridine-3-carboxylate

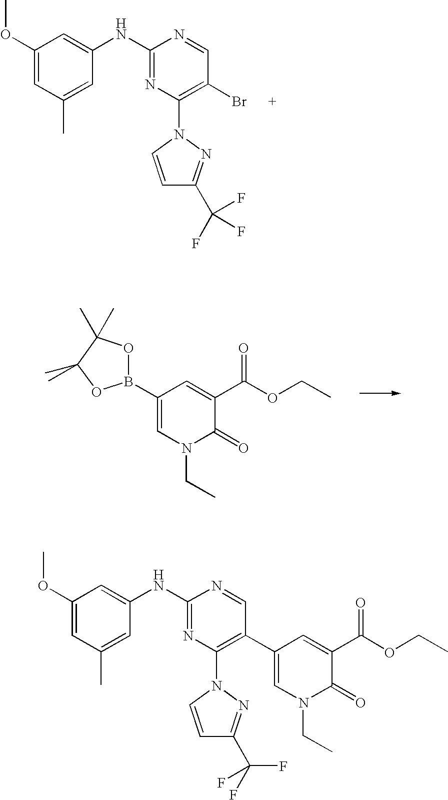

A solution of 5-bromo-N-(3-methoxy-5-methylphenyl)-4-[3-(trifluoromethyl)-1H-pyrazol-1-yl]pyrimidin-2-amine (Intermediate 293, 0.9 mmol, 400 mg), a mixture of ethyl 1-ethyl-2-oxo-5-(4,4,5,5-tetramethyl-1,3,2-dioxaborolan-2-yl)-1,2-dihydropyridine-3-carboxylate and [5-(ethoxycarbonyl)-1-ethyl-6-oxo-1,6-dihydropyridin-3-yl]boronic acid (Intermediate 361, 1.1 mmol based on the boronic ester, 360 mg), [1,1′-bis(diphenylphosphino)ferrocene]dichloropalladium(II)complex with CH₂Cl₂ (0.18 mmol, 146 mg) and sodium carbonate (0.9 mmol, 95 mg) in acetonitrile (40 mL)/water (10 mL) was degassed and heated to 90° C. for 30 minutes under nitrogen. The reaction mixture was concentrated in vacuo. The residue obtained was taken in ethyl acetate, washed with water and brine, dried over sodium sulfate and concentrated. The crude mass was purified by silica gel column chromatography (60-120 mesh) using 2% methanol/chloroform to obtain 350 mg of the title compound.

| Compound | Structure | Mass spectrum and ¹H NMR | SM |
|---|---|---|---|
| Example 1171 | 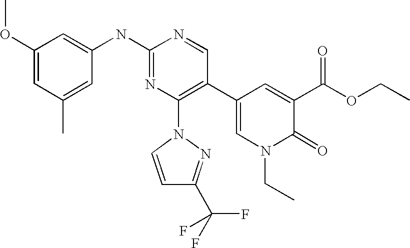 ethyl 1-ethyl-5-{2-[(3-methoxy-5-methylphenyl)amino]- 4-[3-(trifluoromethyl)-1H-pyrazol-1-yl]pyrimidin- 5-yl}-2-oxo-1,2-dihydropyridine-3-carboxylate | MS(ES): 543 (M + 1) for C₂₆H₂₅F₃N₆O₄. 400 MHz, CDCl₃: δ 1.36-1.42 (m, 6H), 2.37 (s, 3H), 3.83 (s, 3H), 4.06 (q, J = 7.20 Hz, 2H), 4.35 (q, J = 7.16 Hz, 2H), 6.52 (s, 1H), 6.70 (d, J = 2.64 Hz, 1H), 6.92 (s, 1H), 7.20 (s, 1H), 7.41 (br s, 1H), 7.56 (d, J = 2.68 Hz, 1H), 7.98 (d, J = 2.72 Hz, 1H), 8.44 (s, 1H), 8.49 (d, J = 1.80 Hz, 1H). | Intermediate 361 and Intermediate 293 5-bromo-N-(3-methoxy-5- methylphenyl)-4-[3- (trifluoromethyl)-1H- pyrazol-1-yl]pyrimidin-2- amine |

### Example 1172

### 1-ethyl-5-{2-[(3-methoxy-5-methylphenyl)amino]-4-[3-(trifluoromethyl)-1H-pyrazol-1-yl]pyrimidin-5-yl}-2-oxo-1,2-dihydropyridine-3-carboxylic acid

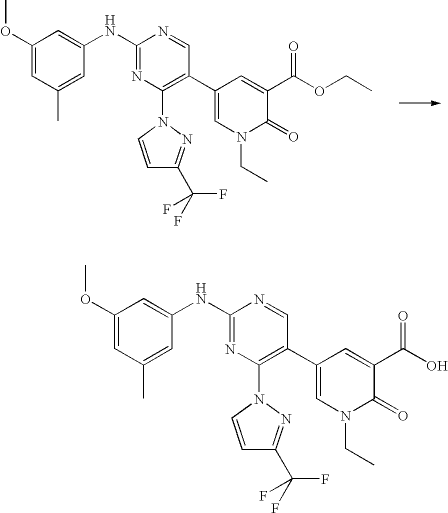

To 350 mg of ethyl 1-ethyl-5-{2-[(3-methoxy-5-methylphenyl)amino]-4-[3-(trifluoromethyl)-1H-pyrazol-1-yl]pyrimidin-5-yl}-2-oxo-1,2-dihydropyridine-3-carboxylate (Example 1171, 0.65 mmol) taken in a mixture of THF (10 mL)/H₂O (10 mL), was added NaOH (1.1 mmol, 46 mg) and stirred at rt for 2 h. The solvent was removed in vacuo and the mixture was then carefully acidified with 1 N HCl and the solid that precipitate was filtered and dried to obtain 200 mg of the title compound.

| Compound | Structure | Mass spectrum and ¹H NMR | SM |
|---|---|---|---|
| Example 1172 | 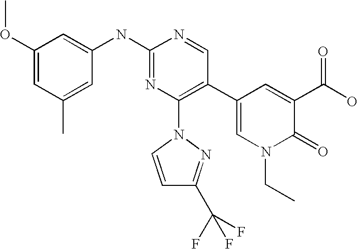 1-ethyl-5-{2-[(3-methoxy-5-methylphenyl)amino]- 4-[3-(trifluoromethyl)-1H-pyrazol-1-yl]pyrimidin- 5-yl}-2-oxo-1,2-dihydropyridine-3-carboxylic acid | MS(ES): 515 (M + 1) for C₂₄H₂₁F₃N₆O₄. 400 MHz, DMSO-d₆: δ 1.31 (t, J = 7.12 Hz, 3H), 2.28 (s, 3H), 3.74 (s, 3H), 4.14 (q, J = 6.92 Hz, 2H), 6.46 (s, 1H), 7.08 (d, J = 2.44 Hz, 1H), 7.14 (s, 1H), 7.32 (s, 1H), 8.10 (d, J = 2.40 Hz, 1H), 8.38 (s, 1H), 8.58 (s, 1H), 8.74 (s, 1H), 10.15 (s, 1H). | Example 1171 ethyl 1-ethyl-5-{2-[(3- methoxy-5- methylphenyl)amino]- 4-[3-(trifluoromethyl)- 1H-pyrazol-1- yl]pyrimidin-5-yl}-2- oxo-1,2- dihydropyridine-3- carboxylate |

### Example 1173

### 5-{2-[(3-methoxy-5-methylphenyl)amino]-4-[3-(trifluoromethyl)-1H-pyrazol-1-yl]pyrimidin-5-yl}-1-methyl-N-(methylsulfonyl)-2-oxo-1,2-dihydropyridine-3-carboxamide

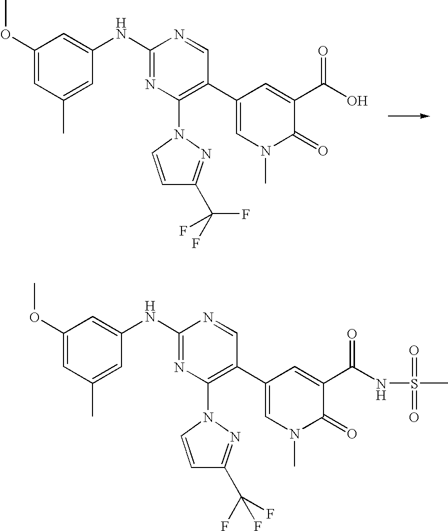

To a solution of 5-{2-[(3-methoxy-5-methylphenyl)amino]-4-[3-(trifluoromethyl)-1H-pyrazol-1-yl]pyrimidin-5-yl}-1-methyl-2-oxo-1,2-dihydropyridine-3-carboxylic acid (Example 859, 0.19 mmol, 100 mg) in CH₂Cl₂ (10 mL), were added methanesulfonamide (0.29 mmol, 28 mg), triethylamine (0.57 mmol, 0.08 mL), 2-chloro-1-methylpyridinium iodide (0.2 mmol, 58 mg) and 4-(Dimethylamino)pyridine (0.04 mmol, 4 mg) and stirred at RT for 2 h. The reaction mixture was diluted with dichloromethane (20 mL) and further washed with 10% citric acid solution (2×15 mL), water (25 mL) and brine (25 mL). The organic layer was dried over Na₂SO₄ and concentrated. The crude mass was purified by flash chromatography (product eluted with 2% MeOH in CHCl₃) to afford 85 mg of the title compound.

| Compound | Structure | Mass spectrum and ¹H NMR | SM |
|---|---|---|---|
| Example 1173 | 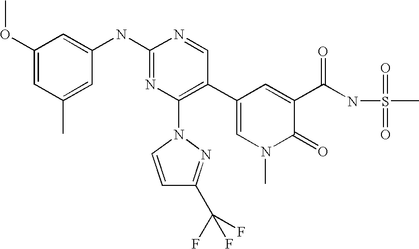 5-{2-[(3-methoxy-5-methylphenyl)amino]-4-[3- (trifluoromethyl)-1H-pyrazol-1-yl]pyrimidin- 5-yl}-1-methyl-N-(methylsulfonyl)-2-oxo-1,2- dihydropyridine-3-carboxylamide | MS(ES): 578 (M + 1) for C₂₄H₂₂F₃N₇O₅S. 400 MHz, DMSO-d₆: δ 2.29 (s, 3H), 3.36 (s, 3H), 3.66 (s, 3H), 3.75 (s, 3H), 6.48 (s, 1H), 7.10 (d, J = 2.64 Hz, 1H), 7.15 (s, 1H), 7.34 (s, 1H), 8.09 (d, J = 2.64 Hz, 1H), 8.45 (d, J = 2.68 Hz, 1H), 8.59 (d, J = 1.76 Hz, 1H), 8.73 (s, 1H), 10.16 (s, 1H), 12.82 (s, 1H). | Example 859 5-{2-[(3-methoxy-5- methylphenyl)amino]-4-[3- (trifluoromethyl)-1H- pyrazol-1-yl]pyrimidin- 5-yl}-1-methyl-2-oxo-1,2- dihydropyridine-3- carboxylic acid |

### Example 1174

### 5-{2-[(3-methoxy-5-methylphenyl)amino]-4-[5-methyl-3-(trifluoromethyl)-1H-pyrazol-1-yl]pyrimidin-5-yl}-1-methyl-N-(methylsulfonyl)-2-oxo-1,2-dihydropyridine-3-carboxamide

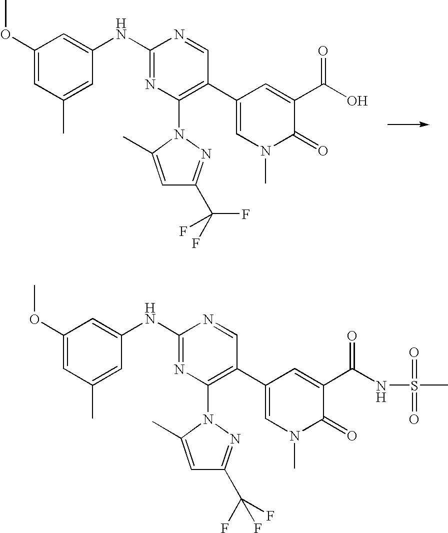

To a solution of 5-{2-[(3-methoxy-5-methylphenyl)amino]-4-[5-methyl-3-(trifluoromethyl)-1H-pyrazol-1-yl]pyrimidin-5-yl-1-methyl-2-oxo-1,2-dihydropyridine-3-carboxylic acid (Example 861, 0.49 mmol, 250 mg) in CH₂Cl₂ (20 mL), was added methanesulfonamide (0.97 mmol, 92 mg), triethylamine (1.45 mmol, 0.21 mL), 2-chloro-1-methylpyridinium iodide (0.58 mmol, 150 mg) and 4-(Dimethylamino)pyridine (0.097 mmol, 12 mg) and stirred at RT for 4 h. The reaction mixture was diluted with dichloromethane (20 mL) and further washed with 1.5 N HCl (2×15 mL), water (25 mL) and brine (25 mL). The organic layer was dried over Na₂SO₄ and concentrated. The crude mass was purified by flash chromatography (product eluted with 1% MeOH in CHCl₃) to afford 120 mg of the title compound.

| Compound | Structure | Mass spectrum and ¹H NMR | SM |
|---|---|---|---|
| Example 1174 | 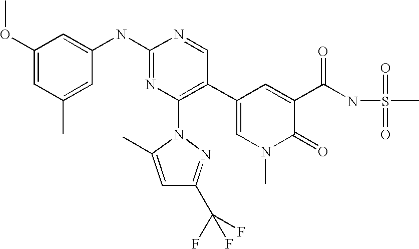 5-{2-[(3-methoxy-5-methylphenyl)amino]-4-[5- methyl-3-(trifluoromethyl)-1H-pyrazol-1-yl]pyrimidin- 5-yl}-1-methyl-N-(methylsulfonyl)-2-oxo-1,2- dihydropyridine-3-carboxamide | MS(ES): 592 (M + 1) for C₂₅H₂₄F₃N₇O₅S. 400 MHz, DMSO-d₆: δ 2.26 (s, 3H), 2.47-2.50 (m, 6H), 3.62 (s, 3H), 3.71 (s, 3H), 6.46 (s, 1H), 6.81 (s, 1H), 7.11 (s, 1H), 7.25 (s, 1H), 7.70 (d, J = 2.68 Hz, 1H), 8.40 (d, J = 2.68 Hz, 1H), 8.86 (s, 1H), 10.15 (s, 1H). | Example 861 5-{2-[(3-methoxy-5- methylphenyl)amino]-4-[5- methyl-3-(trifluoromethyl)- 1H-pyrazol-1-yl]pyrimidin- 5-yl}-1-methyl-2-oxo-1,2- dihydropyridine-3- carboxylic acid |

### Example 1175

### 1-ethyl-5-{2-[(3-methoxy-5-methylphenyl)amino]-4-[3-(trifluoromethyl)-1 1H-pyrazol-1-yl]pyrimidin-5-yl}-N-(methylsulfonyl)-2-oxo-1,2-dihydropyridine-3-carboxamide

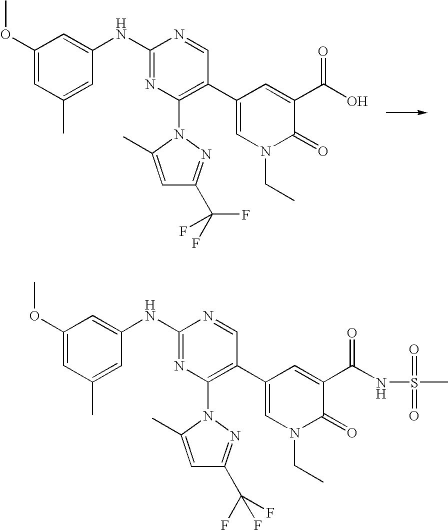

A mixture of 1-ethyl-5-{2-[(3-methoxy-5-methylphenyl)amino]-4-[3-(trifluoromethyl)-1H-pyrazol-1-yl]pyrimidin-5-yl}-2-oxo-1,2-dihydropyridine-3-carboxylic acid (Example 1172, 0.53 mmol, 270 mg), methanesulfonamide (0.78 mmol, 74 mg), triethylamine (1.5 mmol, 0.21 mL), 2-chloro-1-methylpyridinium iodide (0.6 mmol, 153 mg) and 4-(Dimethylamino)pyridine (0.097 mmol, 12 mg) in CH₂Cl₂ (10 mL), was stirred at RT for 2 h. The reaction mixture was diluted with dichloromethane (20 mL) and further washed with 10% citric acid solution (2×15 mL), water (25 mL) and brine (25 mL). The organic layer was dried over Na₂SO₄ and concentrated. The crude mass was purified by flash chromatography (product eluted with 2% MeOH in CHCl₃) to afford 170 mg of the title compound.

| Compound | Structure | Mass spectrum and ¹H NMR | SM |
|---|---|---|---|
| Example 1175 | 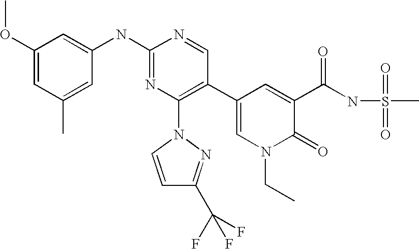 1-ethyl-5-{2-[(3-methoxy-5-methylphenyl)amino]- 4-[3-(trifluoromethyl)-1H-pyrazol-1-yl]pyrimidin- 5-yl}-N-(methylsulfonyl)-2-oxo-1,2- dihydropyridine-3-carboxamide | MS(ES): 592 (M + 1) for C₂₅H₂₄F₃N₇O₅S. 400 MHz, DMSO-d₆: δ 1.29 (t, J = 7.12 Hz, 3H), 2.28 (s, 3H), 3.36 (s, 3H), 3.74 (s, 3H), 4.10 (q, J = 6.28 Hz, 2H), 6.46 (s, 1H), 7.08 (d, J = 2.52 Hz, 1H), 7.14 (s, 1H), 7.33 (s, 1H), 8.15 (d, J = 2.64 Hz, 1H), 8.38 (d, J = 2.60 Hz, 1H), 8.58 (d, J = 1.60 Hz, 1H), 8.75 (s, 1H), 10.16 (s, 1H), 12.84 (s, 1H). | Example 1172 1-ethyl-5-{2-[(3-methoxy- 5-methylphenyl)amino]- 4-[3-(trifluoromethyl)-1H- pyrazol-1-yl]pyrimidin- 5-yl}-2-oxo-1,2- dihydropyridine-3- carboxylic acid |

### Example 1176

### 1-ethyl-5-{2-[(3-methoxy-5-methylphenyl)amino]-4-[5-methyl-3-(trifluoromethyl)-1H-pyrazol-1-yl]pyrimidin-5-yl}-N-(methylsulfonyl)-2-oxo-1,2-dihydropyridine-3-carboxamide

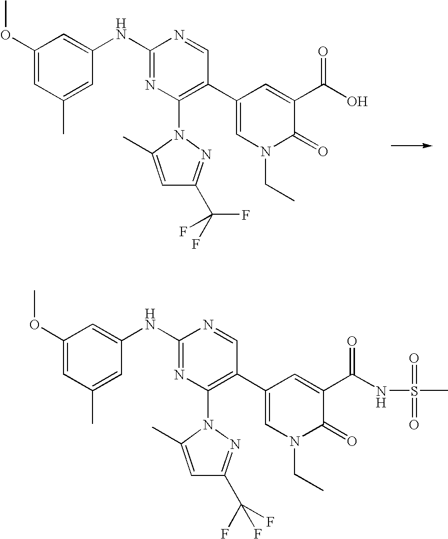

A mixture of 1-ethyl-5-{2-[(3-methoxy-5-methylphenyl)amino]-4-[5-methyl-3-(trifluoromethyl)-1H-pyrazol-1-yl]pyrimidin-5-yl}-2-oxo-1,2-dihydropyridine-3-carboxylic acid (Example 863, 0.59 mmol, 310 mg) methanesulfonamide (0.87 mmol, 83 mg), triethylamine (1.5 mmol, 0.21 mL), 2-chloro-1-methylpyridinium iodide (0.6 mmol, 153 mg) and 4-(Dimethylamino)pyridine (0.097 mmol, 12 mg) in CH₂Cl₂ (10 mL) was stirred at RT for 2 h. The reaction mixture was diluted with dichloromethane (20 mL) and further washed with 10% citric acid (2×15 mL), water (25 mL) and brine (25 mL). The organic layer was dried over Na₂SO₄ and concentrated. The crude mass was purified by flash chromatography (product eluted with 3% MeOH in CHCl₃) to afford 120 mg of the title compound.

| Compound | Structure | Mass spectrum and ¹H NMR | SM |
|---|---|---|---|
| Example 1176 | 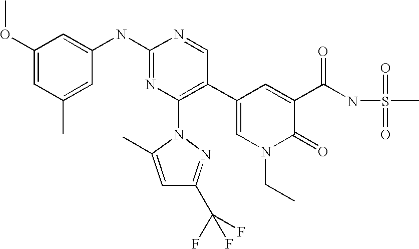 1-ethyl-5-{2-[(3-methoxy-5-methylphenyl)amino]-4- [5-methyl-3-(trifluoromethyl)-1H-pyrazol-1-yl]pyrimidin- 5-yl}-N-(methylsulfonyl)-2-oxo-1,2-dihydropyridine- 3-carboxamide | MS(ES): 606 (M + 1) for C₂₆H₂₆F₃N₇0₅S. 400 MHz, DMSO-d₆: δ 1.25 (t, J = 7.20 Hz, 3H), 2.27 (s, 3H), 2.45 (s, 3H), 3.35 (s, 3H), 3.72 (s, 3H), 4.07 (q, J = 7.00 Hz, 2H), 6.47 (s, 1H), 6.80 (s, 1H), 7.12 (s, 1H), 7.25 (s, 1H), 7.88 (d, J = 2.60 Hz, 1H), 8.26 (d, J = 2.52 Hz, 1H), 8.89 (s, 1H), 10.15 (s, 1H), 12.73 (s, 1H). | Example 863 1-ethyl-5-{2-[(3-methoxy- 5-methylphenyl)amino]-4- [5-methyl-3- (trifluoromethyl)-1H- pyrazol-1-yl]pyrimidin- 5-yl}-2-oxo-1,2- dihydropyridine-3- carboxylic acid |

1. A compound of formula (I): 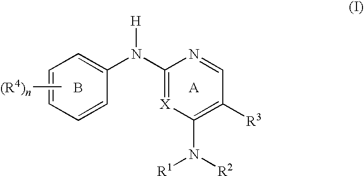
or a pharmaceutically acceptable salt thereof, wherein:
X is CH or N;
R¹ is hydrogen, a C₁₋₆alkyl, C₂₋₆alkenyl, C₂₋₆alkynyl, C₃₋₁₄carbocyclyl, or a heterocyclyl,
wherein R¹ may be optionally substituted on carbon by one or more R⁶; and wherein if said hetercyclyl contains an ═N— or a —S— moiety that nitrogen may be optionally substituted by one oxo group and that sulfur may be optionally substituted by one or two oxo groups; and wherein if said heterocyclyl contains an —NH— moiety that nitrogen may be optionally substituted by a group selected from R⁷; provided that R¹ is not a substituted or unsubstituted phenyl;
R² is hydrogen or a C₁₋₆alkyl; or
R¹ and R², together with the nitrogen to which they are attached, form a heterocyclyl,
wherein said heterocyclyl may be optionally substituted on carbon by one or more R⁸; wherein if said hetercyclyl contains an ═N— or a —S— moiety that nitrogen may be optionally substituted by one oxo group and that sulfur may be optionally substituted by one or two oxo groups; and
wherein if said heterocyclyl contains an —NH— moiety that nitrogen may be optionally substituted by a group selected from R⁹;
R³ is a C₆-₁₄aryl or a heteroaryl; wherein R³ may be optionally substituted on carbon by one or more R¹⁴; and wherein if said heteraryl contains an ═N— or a —S— moiety that nitrogen may be optionally substituted by one oxo group and that sulfur may be optionally substituted by one or two oxo groups; and wherein if said heteroaryl contains an —NH— moiety that nitrogen may be optionally substituted by a group selected from R¹⁵; provided that R³ is not an unsubstituted phenyl or an unsubstituted thiophenyl;
R⁴, for each occurrence, is independently selected from the group consisting of halo, cyano, nitro, hydroxy, C₁₋₆alkyl, C₁₋₆alkoxy, C₁₋₆alkanoyl, carbamoyl, N—C₁₋₆alkylcarbamoyl, N—C₁₋₆alkoxycarbamoyl, N,N—(C₁₋₆alkyl)₂carbamoyl, N—(SO₂R′)carbamoyl, N—C₁₋₆alkyl, C₁₋₆alkyl-S(O)ₐ—, R¹⁷_{R}¹⁸N—S(O)₁—, CH₃₋₁₄carbocyclyl, and heterocyclyl; or two R⁴ taken together with the carbon atoms to which they are attached form a C₃₋₁₄carbocyclyl or a heterocyclyl, wherein each R⁴ may be optionally substituted on carbon by one or more R¹⁶, wherein if either of said heterocyclyl contains an —NH— moiety that nitrogen may be optionally substituted by a group selected from R²⁶; provided that ring B together with —(R⁴)ₙ is not 3,4,5-trimethoxyphenyl;
n is an integer from 1 to 5;
a is 0, 1, or 2;
R⁶, R⁸, and R¹⁴, for each occurrence, are each independently selected from the group consisting of hydroxy, halo, cyano, nitro, C₁₋₆alkyl, C₂₋₆alkenyl, C₂₋₆alkynyl, mercapto, C₁₋₆alkoxy, C₁₋₆alkylS(O)ₐ wherein a is 0 to 2,—C(═N—OH)NH₂, —C(O)NHNH₂, phenoxy, carboxy, oxo, amino, N—C₁₋₆alkylamino, N,N—(C₁₋₆alkyl)₂amino, C₁₋₆alkoxycarbonyl, C₁₋₆alkanoyl, C₁₋₆alkanoyloxy, C₁₋₆alkanoylamino, C₁₋₆alkoxycarbonylamino, carbamoyl, N—C₁₋₆alkylcarbamoyl, N—C₁₋₆alkoxycarbamoyl, N,N‘3(C₁₋₆alkyl)₂carbamoyl, N—C₁₋₆alkyl-N-alkoxycarbamoyl, N—(SO₂R′)carbamoyl, N—C₁₋₆alkyl-N—(SO₂R′)carbamoyl, C₁₋₆alkylsulphonylamino, sulphamoyl, N-(C₁₋₆alkyl)sulphamoyl, N,N-(C₁₋₆alkyl)₂sulphamoyl, sulphamoylamino, N—(C₁₋₆alkyl)sulphamoylamino, N,N—(C₁₋₆alkyl)₂sulphamoylamino, C₃₋₁₄carbocyclyl-L- and heterocyclyl-L-; or two R¹⁴ taken together with the carbon atoms to which they are attached form a C₃₋₁₄carbocyclyl or a heterocyclyl; wherein R⁶, R⁸, and R¹⁴ may be each independently optionally substituted on carbon by one or more R¹⁰; and wherein if said hetercyclyl contains an ═N— or a —S— moiety that nitrogen may be optionally substituted by one oxo group and that sulfur may be optionally substituted by one or two oxo groups; and wherein if either of said heterocyclyl contains an —NH— moiety that nitrogen may be optionally substituted by a group selected from R¹¹;
R′ and R″, for each occurrence, are independently selected from the group consisting of C₁₋₆alkyl, C₆₋₁₄aryl and heterocyclyl, wherein R′ and R″may be optionally substituted on carbon by one or more R²² and wherein if said heterocyclyl contains an —NH— moiety that nitrogen may be optionally substituted by a group selected from R²³;
R⁷, R⁹, R¹⁵ and R²³, for each occurrence, are each independently selected from the group consisting of C₁₋₆alkyl, C₁₋₆alkoxycarbonyl, C₁₋₆alkanoyl, carbamoyl, N—C₁₋₆alkylcarbamoyl, N,N—(C₁₋₆alkyl)₂carbamoyl, C₃₋₁₄carbocyclyl-C(O)—, heterocyclyl-C(O)—, (C₁₋₆alkyl)₃silyl, C₁₋₆alkylS(O)ₐ wherein a is 0 to 2, wherein R⁷, R⁹, and R¹⁵ may be each independently optionally substituted on carbon by one or more R¹²; and wherein if said hetercyclyl contains an ═N— or a —S— moiety that nitrogen may be optionally substituted by one oxo group and that sulfur may be optionally substituted by one or two oxo groups; and wherein if said heterocyclyl contains an —NH— moiety that nitrogen may be optionally substituted by a group selected from R¹³;
L, for each occurrence, is independent selected from a direct bond, —)—, —N(R²⁵)—, —C(O)—, —N(R²⁵)C(O)—, —C(O)N(R²⁵)—, —S(O)ₛ—, —SO₂N(R²⁵)— or —N(R²⁵)SO₂—; wherein R²⁵, for each occurrence, is independently selected from hydrogen or C₁₋₆alkyl and s is 0, 1 or 2;
R¹⁰ and R¹², for each occurrence, are independently selected from the group consisting of C₁₋₆alkyl, phenyl, halo, cyano, nitro, oxo, carboxy, hydroxy, C₁₋₆alkoxy, C₁₋₆alkoxycarbonyl, amino, N—C₁₋₆alkylamino, N,N—(C₁₋₆alkyl)₂amino, C₁₋₆alkanoylamino, C₁₋₆alkylSO₂NH—, carbamoyl, N—C₁₋₆alkylcarbamoyl, N,N—(C₁₋₆alkyl)₂carbamoyl, N—C₁₋₆alkyloxycarbamoyl, C₁₋₆alkylS(O)ₐ wherein a is 0 to 2, and heterocyclyl, wherein said R¹⁰ and R¹² are independently optionally substituted on carbon by one or more C₁₋₆alkyl and wherein if said heterocyclyl contains an —NH— moiety that nitrogen may be optionally substituted by a group selected from R^{13′};
R¹¹, R¹³, R^{13′}, and R²⁶, for each occurrence, are each independently selected from the group consisting of C₁₋₆alkyl, C₁₋₆alkoxycarbonyl, C₁₋₆alkanoyl, C₃₋₆cycloalkanoyl, carbamoyl, C₁₋₆alkanoyloxy, C₁₋₆alkylS(O)ₐ, arylS(O)ₐ wherein a is 0 to 2, carboxy, sulphamoyl a wherein said R¹¹, R¹³, R^{13′}, and R²⁶ are independently optionally substituted on carbon by one or more amino, C₁₋₆alkyl, C₁₋₆alkoxy or heterocyclyl;
R¹⁶, for each occurrence, is independently, a halo, hydroxy, a C₁₋₆alkyl, or a C₁₋₆alkoxy;
R¹⁷ and R¹⁸, for each occurrence, are independently hydrogen or a C₁₋₆alkyl; or R¹⁷ and R¹⁸, together with the nitrogen to which they are attached form a heterocyclyl;
R²², for each occurrence, is independently selected from the group consisting of halo, C₁₋₆alkyl, S(O)ₐR″ wherein a is 0 to 2, C₁₋₆alkanoyl, C₁₋₆alkanoylamino and heterocyclyl wherein may be optionally substituted on carbon by one or more R²⁴;
R²⁴ is selected from halo, C₁₋₆alkanoylamino, and heterocyclyl; provided that —NR¹R² is not —NHCH₃ or —N(CH₃)₂.

2. A compound according to claim 1, or a pharmaceutically acceptable salt thereof, wherein X is N.

3. A compound according to claim 1 or 2, or a pharmaceutically acceptable salt thereof, wherein R¹ and R², together with the nitrogen to which they are attached, form a heterocyclyl, wherein said heterocyclyl may be optionally substituted on carbon by one or more R⁸; wherein if said heterocyclyl contains an ═N— or a —S— moiety that nitrogen may be optionally substituted by one oxo group and that sulfur may be optionally substituted by one or two oxo groups; and wherein if said heterocyclyl contains an —NH— moiety that nitrogen may be optionally substituted by a group selected from R⁹.

4. A compound according to claim 3, or a pharmaceutically acceptable salt thereof, wherein R¹ and R², together with the nitrogen to which they are attached, form a 1H-pyrazol-1-yl, wherein said 1H-pyrazol-1-yl may be optionally substituted on carbon by one or more R⁸.

5. A compound according to any one of claims 1 through 4, or a pharmaceutically acceptable salt thereof, wherein R³ is a heteroaryl; wherein R³ may be optionally substituted on carbon by one or more R¹⁴; and wherein if said heteraryl contains an ═N— or a —S— moiety that nitrogen may be optionally substituted by one oxo group and that sulfur may be optionally substituted by one or two oxo groups; and wherein if said heteroaryl contains an —NH— moiety that nitrogen may be optionally substituted by a group selected from R¹⁵; provided that R³ is not an unsubstituted thiophenyl.

6. A compound according to claim 1 or a pharmaceutically acceptable salt thereof, wherein:
X is N;
R¹ and R², together with the nitrogen to which they are attached, form pyrazol-1-yl wherein said pyrazol-1-yl may be optionally substituted on carbon by one or more R⁸;
R³ is a 6-membered heteroaryl containing at least one nitrogen atom wherein one of the carbon atoms of said 6-membered heteroaryl ring may be optionally substituted with O to form a —(CO)—, and further wherein said 6-membered heteroaryl may be optionally substituted on carbon by one or more R¹⁴ and when one of the carbon atoms of said 6-membered heteroaryl ring is substituted with O to form a —(CO)—, the nitrogen of that 6-membered heteroaryl is substituted by a group selected from R¹⁵;
n is 2;
R⁴, for each occurrence, is independently a halo, C₁₋₆alkyl or C₁₋₆alkoxy;
R⁸, for each occurrence, is independently a C₁₋₆alkyl or a C₃₋₆cycloalkyl wherein said R⁸ is optionally substituted on carbon by one or more fluoro;
R¹⁴, for each occurrence, is independently a carboxy, C₁₋₆alkoxy, C₁₋₃alkylsulphonylcarbamoyl, N—C₁₋₃alkylcarbamoyl, N—C₁₋₃alkoxycarbamoyl, or C₁₋₆alkylS(O)ₐ wherein a is 0, 1 or 2 wherein said R¹⁴ may be optionally substituted on carbon by one or more hydroxy, (C₁₋₃alkyl)₂N, or C₁₋₃alkylsulfonyl; and
R¹⁵, for each occurrence, is independently a C₁₋₆alkyl wherein said C₁₋₆alkyl is optionally substituted by C₁₋₆alkoxy or saturated heterocyclyl.

7. A pharmaceutical composition comprising a compound of any one of claims 1-6, or a pharmaceutically acceptable salt thereof, and a pharmaceutically acceptable excipient or carrier.

8. A method of inhibiting bacterial DNA gyrase and/or bacterial topoisomerase IV in a warm-blooded animal in need of such treatment, comprising administering to the animal an effective amount of a compound of any one of claims 1-6, or a pharmaceutically acceptable salt thereof.

9. A method of producing an antibacterial effect in a warm-blooded animal in need of such treatment, comprising administering to the animal an effective amount of a compound of any one of claims 1-6, or a pharmaceutically acceptable salt thereof.

10. A method of treating a bacterial infection in a warm-blooded animal in need thereof, comprising administering to the animal an effective amount of a compound of any one of claims 1-6, or a pharmaceutically acceptable salt thereof.

11. The method of claim 10, wherein the bacterial infection is selected from the group consisting of community-acquired pneumoniae, hospital-acquired pneumoniae, skin and skin structure infections, acute exacerbation of chronic bronchitis, acute sinusitis, acute otitis media, catheter-related sepsis, febrile neutropenia, osteomyelitis, endocarditis, urinary tract infections and infections caused by drug resistant bacteria such as Penicillin-resistant Streptococcus pneumoniae, methicillin-resistant Staphylococcus aureus, methicillin-resistant Staphylococcus epidermidis and Vancomycin-Resistant Enterococci.
